(19) United States
(12) Patent Application Publication
(10) Pub. No.: US 2025/0215030 A1
(43) Pub. Date: Jul. 3, 2025

(54) STAT MODULATORS AND USES THEREOF

(71) Applicant: Recludix Pharma, Inc., San Diego, CA (US)

(72) Inventors: Neil Bifulco, Sudbury, MA (US); Howard Bregman, Melrose, MA (US); Giovanni Cianchetta, Boxford, MA (US); Brian Hodous, Arlington, MA (US); Samuel K. Reznik, Cambridge, MA (US); Yong Tang, West Roxbury, MA (US); Andrew Tasker, Simi Valley, CA (US); Rishi G. Vaswani, Lexington, MA (US); Ernest Allen Sickmier, Needham, MA (US); John Yeoman, Medford, MA (US); Xia Tian, Cambridge, MA (US)

(21) Appl. No.: 18/841,804

(22) PCT Filed: Mar. 30, 2023
(86) PCT No.: PCT/US2023/065171

§ 371 Date: Aug. 27, 2024

Related U.S. Application Data
(60) Provisional application No. 63/337,388, filed on May 2, 2022.
(60) Provisional application No. 63/325,908, filed on Mar. 31, 2022.

(51) Int. Cl. C07F 9/6561; A61K 31/675

(52) U.S. Cl. CPC C07F 9/6561; A61K 31/675

(57) ABSTRACT

Provided are compounds of Formula (I) and pharmaceutically acceptable salts and compositions thereof, which are useful for treating a variety of conditions associated with STAT3 and/or STAT6.

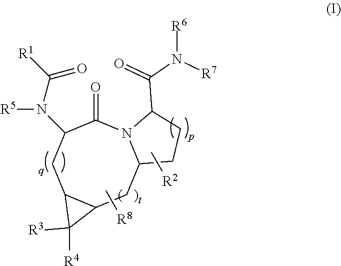

### RELATED APPLICATIONS

This application claims priority to U.S. provisional application No. 63/325,908, filed Mar. 31, 2022 and U.S. provisional application No. 63/337,388, filed May 2, 2022, the entire contents of each of which are incorporated herein by reference.

### BACKGROUND

The Signal Transducer and Activator of Transcription (STAT) family of proteins consists of transcription factors that play an essential role in the regulation of cell processes, such as proliferation, differentiation, apoptosis and angiogenesis. Seven STAT genes have been identified in the human genome: STAT1, STAT2, STAT3, STAT4, STAT5a, STAT5b, and STAT6.

STAT3 has received particular attention because it is strongly associated with the promotion of tumor growth and immune evasion, and the only STAT family member whose genetic deletion results in embryonic lethality. Indeed, aberrantly elevated STAT3 activity has been estimated to occur in more than 70% of human cancers. Activated STAT3 mediates critical gene expression changes and molecular events that dysregulate cell growth and apoptosis, promote angiogenesis, invasion, metastasis, and the development of resistance to apoptosis, and suppress the host's immune surveillance of the tumor, thereby making constitutively-active STAT3 a critical mediator of carcinogenesis and tumor progression.

Another STAT protein that has gained recent interest is STAT6. Recent studies have shown that STAT6 signaling is essential for IL-4- and IL-13-induced epithelial mesenchymal transition (EMT) and aggressiveness of colorectal cancer cells (CRC) cells. STAT6 is involved in several aspects of inflammatory disease and other related conditions.

Given their role in the regulation of cell processes, modulating the activity of one or more STAT proteins, particularly STAT3 and/or STAT6, represent a pivotal area of investigation for the treatment of cancer, inflammatory conditions, and other therapeutic needs.

### SUMMARY

Provided herein are modulators of STAT3 and/or STAT6. Such modulators include those having the Formula I:

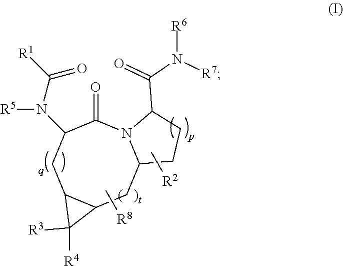

and pharmaceutically acceptable salts and compositions thereof, wherein R¹, R², R³, R⁴, R⁵, R⁶, R⁷, R⁸, q, t and p are as described herein.

In one aspect, the disclosed compounds of Formula I and pharmaceutically acceptable salts thereof inhibit STAT3 and/or STAT6, and are useful in a variety of therapeutic applications such as, for example, in treating cancer and inflammatory conditions.

Pharmaceutical compositions comprising the compounds and pharmaceutically acceptable salts of the disclosed compounds of Formula I, as well as methods for their preparation are also included.

Methods of treating conditions responsive to the modulation of STAT3 and/or STAT6 using the disclosed compounds, pharmaceutically acceptable salts, and compositions thereof are also included.

### DETAILED DESCRIPTION

### 1. General Description of Compounds

In a first embodiment, provided herein is a compound having the structural formula I:

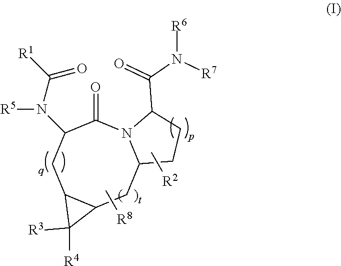

or a pharmaceutically acceptable salt thereof, wherein:
q is 0 or 1 and t is 0, 1, or 2, provided that at least one of q or t is 1;
p is 1 or 2;
R¹ is selected from an 8- to 10-membered fused bicyclic heteroaryl substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}]; an 8- to 10-membered fused bicyclic heterocyclyl substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}]; an aryl substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}]; a —(C₁-C₄)alkyl(aryl) wherein said aryl portion of —(C₁-C₄)alkyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], or —CR^{1a}R^{2a}P(O)[NH(AA)C(O)OR^{T}]][NH(AA)C(O)OR^{T}], —P(O)[OR^{1b}][NH(AA)C(O)OR^{T}]; and a —(C₂-C₄)alkenyl(aryl) wherein said aryl portion of —(C₂-C₄)alkenyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}];
R^{1a} and R^{2a} are each independently selected from hydrogen, cyano, (C₁-C₄)alkyl, hydroxy(C₁-C₄)alkyl and fluoro; or R^{1a} and R^{2a} taken together with the carbon they are attached form oxo;
R^{1b} and R^{2b} are each independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkyl]-O—[(C₁-C₂₀)alkyl], —[(C₁-C₄)alkyl]-OC(O)-[halo(C₁-C₄)alkyl], [(C₁-C₄)alkyl]-OC(O)O-[5- to 7-membered heterocyclyl], [(C₁-C₄)alkyl]-OC(O)-[5- to 7-membered heterocyclyl], —[(C₁-C₄)alkyl]-OC(O)—[(C₁-C₄)alkyl]-OH, —[(C₁-C₄)alkyl]-OC(O)—[(C₁-C₄)alkyl]-O—[(C₁-C₄)alkyl], —[(C₁-C₄)alkyl]-OC(O)O—[(C₁-C₄)alkyl], —[(C₁-C₄)alkyl]-OC(O)O-[halo(C₁-C₄)alkyl], —[(C₁-C₄)alkyl]-OC(O)O—[(C₁-C₄)alkyl]-OH, —[(C₁-C₄)alkyl]-OC(O)O—[(C₁-C₄)alkyl]-O—[(C₁-C₄)alkyl], —[(C₁-C₄)alkylphenyl]-C(O)O—[(C₁-C₄)alkyl], —[(C₁-C₄)alkyl]-OC(O)—[NH(AA)C(O)OR^{T}], —[(C₁-C₄)alkyl]-SC(O)—[(C₁-C₄)alkyl], —[(C₁-C₄)alkyl]-SC(O)-[halo(C₁-C₄)alkyl], —[(C₁-C₄)alkyl]-SC(O)—[(C₁-C₄)alkyl]-OH, —[(C₁-C₄)alkyl]-SC(O)—[(C₁-C₄)alkyl]-O—[(C₁-C₄)alkyl], —[(C₁-C₄)alkyl]-OC(O)NH(C₁-C₄)alkyl], —[(C₁-C₄)alkyl]-OC(O)N[(C₁-C₄)alkyl]₂, and aryl, wherein said 5- to 6-membered heteroaryl and aryl are each optionally and independently substituted with, as valency permits, 1 to 2 groups selected from halo, cyano, and (C₁-C₄)alkyl and wherein said 5- to 7-membered heterocyclyl of [(C₁-C₄)alkyl]-OC(O)O-[5- to 7-membered heterocyclyl] and [(C₁-C₄)alkyl]-OC(O)-[5- to 7-membered heterocyclyl] are each optionally and independently substituted with, as valency permits 1 to 2 groups selected from C(O)OR^{h};
R² is selected from hydrogen, halo, (C₁-C₄)alkyl, halo(C₁-C₄)alkyl, (C₁-C₄)alkoxy, halo(C₁-C₄)alkoxy, hydroxy(C₁-C₄)alkyl, cyano, and hydroxyl;
R³ and R⁴ are each independently selected from hydrogen, halo, and (C₁-C₄)alkyl;
R⁵ and R⁶ are each independently selected from hydrogen, phenyl, and (C₁-C₄)alkyl;
R⁷ is selected from (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, 1 to 3 groups selected from R^{Q};
R⁸ is hydrogen or (C₁-C₄)alkyl;
AA is the residue of an alpha or beta natural or non-natural amino acid;
R^{T} and R^{Ty} are each independently selected from (C₁-C₄)alkyl, (C₁-C₄)alkyl-C(O)O(C₁-C₄)alkyl, benzyl, and phenyl, wherein said phenyl is optionally substituted with 1 or 2 groups selected from halo, (C₁-C₄)alkyl, and halo(C₁-C₄)alkyl;
R^{Q} is selected from halo, (C₂-C₄)alkenyl, (C₁-C₄)alkyl, (C₁-C₄)alkoxy, halo(C₁-C₄)alkoxy, cyano, phenyl, hydroxyl, 4- to 9-membered monocyclic or bicyclic heterocyclyl, 5- to 10-membered monocyclic or bicyclic heteroaryl, (C₃-C₆)cycloalkyl, oxo, imino, —OR^{e}, —C(O)R^{g}, —C(O)OR^{e}, —NR^{c}C(O)R^{e}, —C(O)NR^{c}R^{d}, —NR^{a}R^{b}, —S(O)R^{e}R^{f}, —S(O)₂R^{f}, —S(O)═NH(C₁-C₄)alkyl, —S(O)NR^{e}R^{f}, and —S(O)₂NR^{e}R^{f}, wherein said (C₂-C₄)alkenyl and (C₁-C₄)alkyl are each optionally and independently substituted with, as valency permits, 1 to 3 groups selected from R^{M}, and wherein said phenyl, 5- to 10-membered monocyclic or bicyclic heteroaryl, (C₃-C₆)cycloalkyl, and 4- to 9-membered monocyclic or bicyclic heterocyclyl are each optionally and independently substituted with, as valency permits, 1 to 3 groups selected from R^{F};
R^{Y} is selected from halo, (C₁-C₄)alkoxy, halo(C₁-C₄)alkoxy, cyano, —C(O)R^{g}, —C(O)OR^{e}, —NHC(O)R^{e}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{M} and R^{J} are each independently selected from halo, (C₁-C₄)alkyl, (C₁-C₄)alkoxy, halo(C₁-C₄)alkoxy, cyano, —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R^{f}, —S(O)NR^{e}R^{f}, —S(O)=NR^{e}(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{F}, R^{X}, and R^{Z} are each independently selected from halo, cyano, (C₁-C₄)alkyl, cyano(C₁-C₄)alkyl, C₃-C₆cycloalkyl), halo(C₁-C₄)alkyl, —(C₁-C₄)alkylC(O)NR^{c}R^{d}, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, 5- to 6-membered monocyclic heteroaryl —S(O)R^{e}R^{f}, —S(O)₂R^{f}, —S(O)═NH(C₁-C₄)alkyl, —S(O)NR^{e}R^{f}, —S(O)₂NR^{e}R^{f}, —C(O)OR^{e}, —NR^{c}C(O)R^{e}, —(C₁-C₄alkyl)C(O)R^{g}, —C(O)R^{g}, —C(O)NR^{c}R^{d}, NO₂, and —NR^{a}R^{b}, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, —(C₁-C₄)alkyl(C₁-C₄)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo; and
R^{a}, R^{b}, R^{c}, R^{d}, R^{e}, R^{f}, R^{g}, and R^{h} are each independently selected from, as valency permits, hydrogen, (C₁-C₄)alkyl, (C₂-C₄)alkynyl, —(C₁-C₄)alkylphenyl, phenyl, (C₃-C₆)cycloalkyl, 4- to 6-membered heterocyclyl and 5- to 6-membered heteroaryl, wherein said (C₁-C₄)alkyl is optionally substituted with, as valency permits, 1 to 3 groups selected from R^{J}, and said phenyl, (C₃-C₆)cycloalkyl, 4- to 6-membered heterocyclyl, and 5- to 6-membered heteroaryl are each independently optionally substituted with, as valency permits, 1 to 3 groups selected from halo, cyano, (C₁-C₄)alkyl, halo(C₁-C₄)alkyl, (C₁-C₄)alkoxy, halo(C₁-C₄)alkoxy, hydroxyl, phenyl, and benzyl.

### 2. Definitions

When used in connection to describe a chemical group that may have multiple points of attachment, a hyphen (-) designates the point of attachment of that group to the variable to which it is defined. For example, —NR^{c}C(O)R^{e} means that the point of attachment for this group occurs on the nitrogen atom.

The terms “halo” and “halogen” refer to an atom selected from fluorine (fluoro, —F), chlorine (chloro, —Cl), bromine (bromo, —Br), and iodine (iodo, —I).

Unless otherwise specified, the term “alkyl” when used alone or as part of a larger moiety, such as “haloalkyl”, and the like, means saturated straight-chain or branched monovalent hydrocarbon radical.

The term “haloalkyl” includes mono, poly, and perhaloalkyl groups where the halogens are independently selected from fluorine, chlorine, bromine, and iodine.

“Alkoxy” means an alkyl radical attached through an oxygen linking atom, represented by —O-alkyl. For example, “(C₁-C₄)alkoxy” includes methoxy, ethoxy, proproxy, and butoxy.

“Haloalkoxy” is a haloalkyl group which is attached to another moiety via an oxygen atom such as, e.g., —OCHF₂ or —OCF₃.

The term “oxo” means the group ═O.

The term “imino” means the group ═NH.

Unless otherwise specified, the term “heteroaryl” refers to a 5- to 12-membered aromatic radical containing 1-4 heteroatoms selected from N, O, and S. In some instances, nitrogen atoms in a heteroaryl may be quaternized. The term “heteroaryl” may be used interchangeably with the terms “heteroaryl ring”, “heteroaryl group”, or “heteroaromatic”. A heteroaryl group may be mono- or bi-cyclic. Monocyclic heteroaryl includes, for example, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, etc. Bi-cyclic heteroaryls include groups in which a monocyclic heteroaryl ring is fused to one or more aryl or heteroaryl rings. Nonlimiting examples include indolyl, benzooxazolyl, benzooxodiazolyl, indazolyl, benzimidazolyl, benzthiazolyl, benzothiopheneyl, quinolinyl, quinazolinyl, quinoxalinyl, pyrrolopyridinyl, pyrrolopyrimidinyl, pyrrolopyridinyl, thienopyridinyl, thienopyrimidinyl, indolizinyl, purinyl, cinnolinyl, naphthyridinyl, and pteridinyl. It will be understood that when specified, optional substituents on a heteroaryl group may be present on any substitutable position and, include, e.g., the position at which the heteroaryl is attached (where valency permits).

Unless otherwise specified, the term “heterocyclyl” means a 4- to 12-membered saturated or partially unsaturated heterocyclic ring containing 1 to 4 heteroatoms independently selected from N, O, and S. The terms “heterocycle”, “heterocyclyl”, “heterocyclyl ring”, “heterocyclic group”, “heterocyclic moiety”, and “heterocyclic radical”, are used interchangeably herein. A heterocyclyl ring can be attached to its pendant group at any heteroatom or carbon atom that results in a stable structure. A heterocyclyl group may be mono- or bicyclic (e.g., a bridged, fused, or spiro bicyclic ring). Examples of monocyclic saturated or partially unsaturated heterocyclic radicals include, without limitation, azetidinyl, tetrahydrofuranyl, tetrahydrothienyl, terahydropyranyl, pyrrolidinyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, tetrahydropyrimidinyl, dihydrooxadizolyl, and dihydroisoxazolyl. Bi-cyclic heterocyclyl groups include, e.g., unsaturated heterocyclic radicals fused to another unsaturated heterocyclic radical, cycloalkyl, aryl, or heteroaryl ring, such as for example, benzodioxolyl, dihydrobenzodioxinyl, dihydrobenzofuranyl, dihydrobenzothiophenyl, 5-oxa-2,6-diazaspiro[3.4]oct-6-enyl, 6-thia-2,7-diazaspiro[3.4]octanyl, 2,6-diazaspiro[3.3]heptanyl, spiro[indoline-3,3′-pyrrolidine]-yl, thiochromanyl, and the like. It will be understood that when specified, optional substituents on a heterocyclyl group may be present on any substitutable position and, include, e.g., the position at which the heterocyclyl is attached (where valency permits).

The term “spiro” refers to two rings that shares one ring atom (e.g., carbon).

The term “fused” refers to two rings that share two adjacent ring atoms with one another.

The term “bridged” refers to two rings that share three adjacent ring atoms with one another.

The term “aryl” refers to an aromatic carbocyclic single ring or two fused ring system containing 6 to 10 carbon atoms. Examples include phenyl, indanyl, tetrahydronaphthalene, and naphthyl. In one aspect, the aryl is phenyl or naphthyl.

The terms “cycloalkyl”, used alone or as part of a larger moiety, refers to a saturated cyclic aliphatic monocyclic or bicyclic ring system, as described herein, having from, unless otherwise specified, 3 to 10 carbon ring atoms. Monocyclic cycloalkyl groups include, without limitation, cyclopropyl, cyclobutyl, cyclopentyl, cyclopentenyl, cyclohexyl, cyclohexenyl, cycloheptyl, cycloheptenyl, and cyclooctyl. It will be understood that when specified, optional substituents on a cycloalkyl or cycloaliphatic group may be present on any substitutable position and, include, e.g., the position at which the cycloalkyl group is attached.

The “residue of an amino acid” is the moiety remaining after formation of a bond between a reactive group in another compound (e.g., an amino group) and the carboxylic acid in the amino acid, after formation of a bond between a reactive group in another compound (e.g., a carboxylic acid) and the amino group in the amino acid, or both. As a consequence of the bond(s) formation, the carboxylic acid in the amino acid no longer has the OH group and instead has a bond between the carbonyl group and the reactive group in the compound; the amino group has only one hydrogen atom and instead has a bond between the reactive group in the other compound and the nitrogen of the amino group; or both. For example, the “residue of an alpha amino acid” can be depicted structurally as NH₂CR′R—C(O)—, —NHCR′R—C(O)OH or —NHCR′R—C(O)—; and the “residue of an beta amino acid” can be depicted structurally as or NH₂CR′RCH₂—C(O)—, —NHCR′RCH₂—C(O)OH or —NHCR′RCH₂—C(O)—, where R′ is H or C₁-C₆ alkyl and R is H or C₁-C₆ alkyl optionally substituted with 1 to 3 groups selected from halo, (C₁-C₃)alkoxy, OH, NH₂, —NH(C₁-C₄ alkyl), —N[(C₁-C₄ alkyl)]₂, SH, S(C₁-C₄ alkyl), imino, COOH, —COO(C₁-C₄ alkyl), —CO(C₁-C₄ alkyl), —CONH(C₁-C₄ alkyl)phenyl, phenyl, and 5- to 10-membered heteroaryl, wherein said C₁-C₆ alkyl may also be optionally interrupted by a sulfur or nitrogen heteroatom and wherein said phenyl is optionally substituted with 1 to 3 groups selected from OH, cyano, (C₁-C₄ alkyl), and halo(C₁-C₄ alkyl); or R is taken together with the nitrogen atoms from the alpha or beta amino acid residue to form a 4- to 6-membered heterocyclyl. For naturally occurring alpha amino acid (i.e., amino acids that occur in nature), R′ is H and R is selected from hydrogen, methyl, isopropyl, —CH₂CH(CH₃)₂, —(CH₂)₂SCH₃, —CH(CH₃)(CH₂CH₃), CH₂OH, —CH(OH)(CH₃), CH₂SH, —CH₂C(O)NH₂, —(CH₂)₂C(O)NH₂, benzyl, p-hydroxybenzyl, —CH₂(indolyl), —(CH₂)₄NH₂, —(CH₂)₃NHC(═NH₂)NH₂, —CH₂(imidazolyl), —(CH₂)COOH, and —(CH₂)₂COOH; or R taken together with the nitrogen atom of the alpha or beta amino acid residue forms a pyrrolidinyl ring.

Non-natural amino acids are known in the art and include e.g., alpha-alkyl amino acids (e.g., alpha methyl), alpha-alkylalkoxy amino acids (e.g., alpha —CH₂OCH₃), N-methyl amino acids, homo-amino acids, etc.

Compounds having one or more chiral centers can exist in various stereoisomeric forms. Stereoisomers are compounds that differ only in their spatial arrangement. Stereoisomers include all diastereomeric, enantiomeric, and epimeric forms as well as racemates and mixtures thereof. A “geometric isomer” refers to isomers that differ in the orientation of substituent group in relationship to a carbon-carbon double bond, a cycloalkyl ring, or a bridged bicyclic system. Atoms (other than H) on each side of a carbon-carbon double bond may be in an E (substituents are on opposite sides of the carbon-carbon double bond) or Z (substituents are oriented on the same side) configuration. “Cis” refers to substituents oriented on the same side of the ring, whereas “trans” refers to substituents oriented on opposite sides of the ring.

When the stereochemical configuration at a chiral center in a compound having one or more chiral centers is depicted by its chemical name (e.g., where the configuration is indicated in the chemical name by “R” or “S”) or structure (e.g., the configuration is indicated by “wedge” bonds), the enrichment of the indicated configuration relative to the opposite configuration is greater than 50%, 60%, 70%, 80%, 90%, 99% or 99.9%. “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

When a geometric isomer is depicted by name or structure, the enrichment of the indicated isomer relative to the opposite isomer is greater than 50%, 60%, 70%, 80%, 90%, 99% or 99.9%. “Enrichment of the indicated isomer relative to the opposite isomer” is a mole percent and is determined by dividing the number of compounds with the indicated geometrical configuration by the total number of all of the compounds with the same or opposite geometrical configuration in a mixture.

When a disclosed compound is named or depicted by structure without indicating stereochemistry, it is understood that the name or the structure encompasses one of the possible stereoisomers or geometric isomers free of the others, or a mixture of the encompassed stereoisomers or geometric isomers.

In certain instances, compounds were isolated and tested as a 1:1 mixture of diastereomers. In such cases, the relative stereochemistry is denoted by the term “rel-” and by the use of flat bonds instead of wedges. For example,

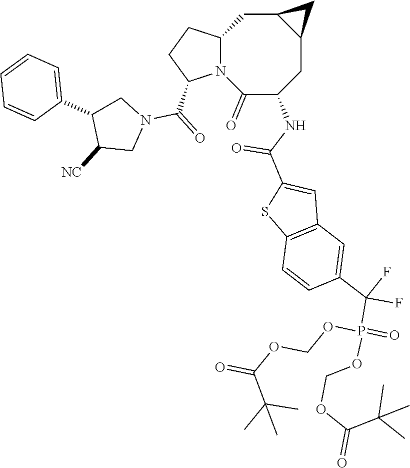 [SMILES: CC(C)(C)C(=O)OCOP(=O)(OCOC(=O)C(C)(C)C)C(F)(F)c1ccc2sc(C(=O)N[C@H]3C[C@@H]4C[C@@H]4C[C@H]4CC[C@@H](C(=O)N5C[C@@H](C#N)[C@H](c6ccccc6)C5)N4C3=O)cc2c1]

### ((((2-(((3S,6S,7aS,8aR,9aR)-3-((rel-trans)-3-cyano-4-phenylpyrrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(methylene) bis(2,2-dimethylpropanoate)

means that the substituents about the pyrrolidine ring are trans and encompass a mixture of both diastereomers

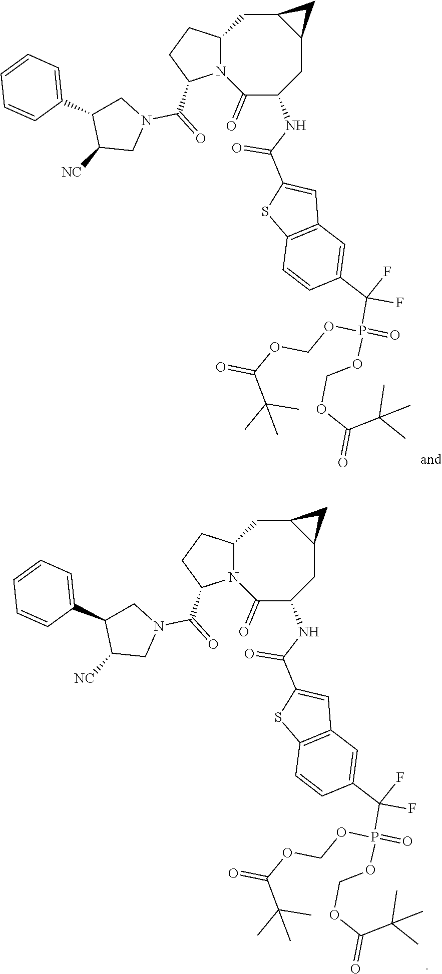 [SMILES: CC(C)(C)C(=O)OCOP(=O)(OCOC(=O)C(C)(C)C)C(F)(F)c1ccc2sc(C(=O)N[C@H]3C[C@@H]4C[C@@H]4C[C@H]4CC[C@@H](C(=O)N5C[C@@H](C#N)[C@H](c6ccccc6)C5)N4C3=O)cc2c1 ; CC(C)(C)C(=O)OCOP(=O)(OCOC(=O)C(C)(C)C)C(F)(F)c1ccc2sc(C(=O)N[C@H]3C[C@@H]4C[C@@H]4C[C@H]4CC[C@@H](C(=O)N5C[C@H](c6ccccc6)[C@@H](C#N)C5)N4C3=O)cc2c1]

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The term “inhibit,” “inhibition” or “inhibiting” includes a decrease in the baseline activity of a biological activity or process.

As used herein, the terms “treatment,” “treat,” and “treating” refer to reversing, alleviating, delaying the onset of, or inhibiting the progress of a disease or disorder, or one or more symptoms thereof, as described herein. In some aspects, treatment may be administered after one or more symptoms have developed, i.e., therapeutic treatment. In other aspects, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “effective amount” or “therapeutically effective amount” refers to an amount of a compound described herein that is sufficient to achieve the desired therapeutic effect (such as treatment of a condition recited herein) under the conditions of administration e.g., a dosage of between 0.01-100 mg/kg body weight/day.

### 3. Compounds

In a first embodiment, provided is a compound of Formula I:

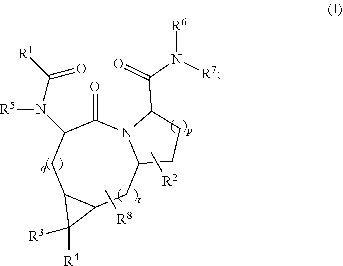

or a pharmaceutically acceptable salt thereof, wherein the variables are as described above. Alternatively, as part of the first embodiment, R^{F}, R^{X}, and R^{Z} are each independently selected from halo, cyano, (C₁-C₄)alkyl, cyano(C₁-C₄)alkyl, (C₃-C₆)cycloalkyl, halo(C₁-C₄)alkyl, —(C₁-C₄)alkylC(O)NR^{c}R^{d}, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, 5- to 6-membered monocyclic heteroaryl —S(O)R^{e}R^{f}, —S(O)₂R^{f}, —S(O)═NH(C₁-C₄)alkyl, —S(O)NR^{e}R^{f}, —S(O)₂NR^{e}R^{f}, —C(O)OR^{e}, —NR^{c}C(O)R^{e}, —(C₁-C₄alkyl)C(O)R^{g}, —C(O)R^{g}, —C(O)NR^{c}R^{d}, NO₂, and —NR^{a}R^{b}, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo, and the remaining variables are as described above.

In a second embodiment, the compound of Formula I is of the Formula II:

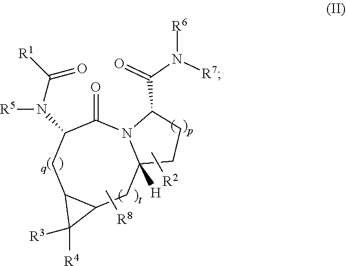

or a pharmaceutically acceptable salt thereof, wherein the variables are as described above for Formula I.

In a third embodiment, q in the compound of Formula I or II, or a pharmaceutically acceptable salt thereof is q, wherein the remaining variables are as described above for Formula I.

In a fourth embodiment, R² in the compound of Formula I or II, or a pharmaceutically acceptable salt thereof is hydrogen, wherein the remaining variables are as described above for Formula I or the third embodiment.

In a fifth embodiment, the compound of Formula I is of the Formula III or IV

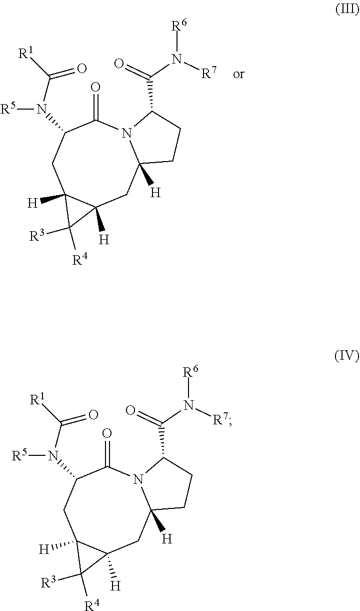

or a pharmaceutically acceptable salt thereof, wherein the variables are as described above for Formula I.

In a sixth embodiment, R⁵ in the compound of Formula I, II, III, or IV, or a pharmaceutically acceptable salt thereof, is hydrogen, wherein the remaining variables are as described for Formula I or any one of the third or fourth embodiments.

In a seventh embodiment, R³ and R⁴ in the compound of Formula I, II, III, or IV, or a pharmaceutically acceptable salt thereof, are each independently selected from hydrogen and halo, wherein the remaining variables are as described for Formula I or any one of the third, fourth, or sixth embodiments. Alternatively, as part of a seventh embodiment, R³ and R⁴ in the compound of Formula I, II, III, or IV, or a pharmaceutically acceptable salt thereof, are each hydrogen, wherein the remaining variables are as described for Formula I or any one of the third, fourth, or sixth embodiments. Alternatively, as part of a seventh embodiment, R³ and R⁴ in the compound of Formula I, II, III, or IV, or a pharmaceutically acceptable salt thereof, are each fluoro, wherein the remaining variables are as described for Formula I or any one of the third, fourth, or sixth embodiments.

In an eighth embodiment, R¹ in the compound of Formula I, II, III, or IV, or a pharmaceutically acceptable salt thereof, is selected from 8- to 10-membered fused bicyclic heteroaryl and aryl, each of which are substituted with —CR^{1a}R^{2a}P(O)OR^{1b}OR^{2b}, —CR^{1a}R^{2a}P(O)[NHR^{Ty}][NH(AA)C(O)OR^{T}], —CR^{1a}R^{2a}P(O)[NH(AA)C(O)OR^{T}]][NH(AA)C(O)OR^{T}], or —CR^{1a}R^{2a}P(O)[OR^{1b}][NH(AA)C(O)OR^{T}], wherein the remaining variables are as described for Formula I or any one of the third, fourth, sixth, or seventh embodiments. Alternatively, as part of an eighth embodiment, R¹ in the compound of Formula I, II, III, or IV, or a pharmaceutically acceptable salt thereof, is selected from benzothiophenyl and naphthalenyl, each of which are substituted with —CR^{1a}R^{2a}P(O)OR^{1b}OR^{2b}, —CR^{1a}R^{2a}P(O)[NHR^{Ty}][NH(AA)C(O)OR^{T}], —CR^{1a}R^{2a}P(O)[NH(AA)C(O)OR^{T}]][NH(AA)C(O)OR^{T}], or —CR^{1a}R^{2a}P(O)[OR^{1b}][NH(AA)C(O)OR^{T}], wherein the remaining variables are as described for Formula I or any one of the third, fourth, sixth, or seventh embodiments. Alternatively, as part of an eighth embodiment, R¹ in the compound of Formula I, II, III, or IV, or a pharmaceutically acceptable salt thereof, is selected from

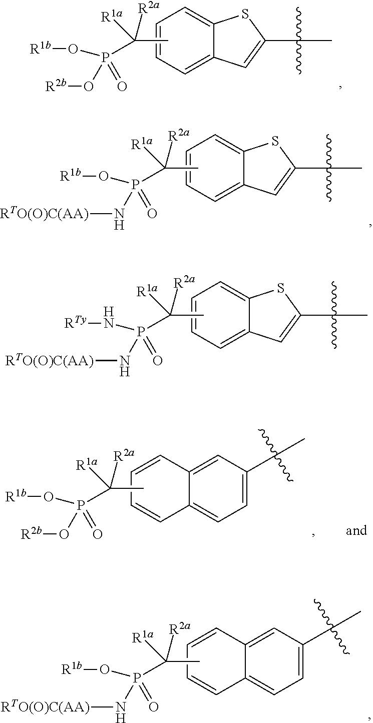

wherein the remaining variables are as described for Formula I or any one of the third, fourth, sixth, or seventh embodiments. Alternatively, as part of an eighth embodiment, R¹ in the compound of Formula I, II, III, or IV, or a pharmaceutically acceptable salt thereof, is selected from

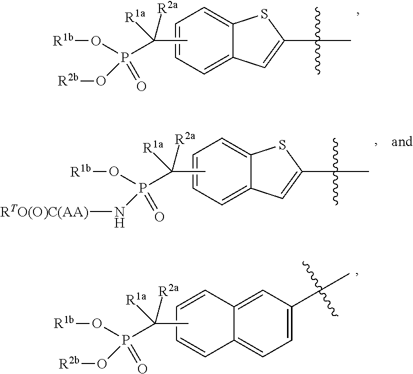

wherein the remaining variables are as described for Formula I or any one of the third, fourth, sixth, or seventh embodiments. Alternatively, as part of an eighth embodiment, R¹ in the compound of Formula I, II, III, or IV, or a pharmaceutically acceptable salt thereof, is

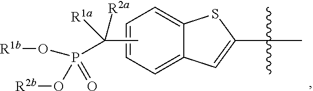

wherein the remaining variables are as described for Formula I or any one of the third, fourth, sixth, or seventh embodiments.

In a ninth embodiment, R^{1a} and R^{2a} in the compound of Formula I, II, III, or IV, or a pharmaceutically acceptable salt thereof, are each independently selected from hydrogen and fluoro, wherein the remaining variables are as described for Formula I or any one of the third, fourth and sixth to eighth embodiments. Alternatively, as part of a ninth embodiment, R^{1a} and R^{2a} in the compound of Formula I, II, III, or IV, or a pharmaceutically acceptable salt thereof, are each hydrogen, wherein the remaining variables are as described for Formula I or any one of the third, fourth and sixth to eighth embodiments. Alternatively, as part of a ninth embodiment, R^{1a} in the compound of Formula I, II, III, or IV, or a pharmaceutically acceptable salt thereof, is hydrogen and R^{2a} is fluoro, wherein the remaining variables are as described for Formula I or any one of the third, fourth and sixth to eighth embodiments. Alternatively, as part of a ninth embodiment, R^{1a} and R^{2a} in the compound of Formula I, II, III, or IV, or a pharmaceutically acceptable salt thereof, are each fluoro, wherein the remaining variables are as described for Formula I or any one of the third, fourth and sixth to eighth embodiments.

In a tenth embodiment, R^{1b} and R^{2b} in the compound of Formula I, II, III, or IV, or a pharmaceutically acceptable salt thereof, are each independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkylphenyl]-C(O)O—[(C₁-C₄)alkyl], —[(C₁-C₄)alkyl]-OC(O)—[NH(AA)C(O)OR^{T}], —[(C₁-C₄)alkyl]-OC(O)—[(C₁-C₄)alkyl]-OH, —[(C₁-C₄)alkyl]-OC(O)O-[5- to 7-membered heterocyclyl], —[(C₁-C₄)alkyl]-OC(O)O—[(C₁-C₄)alkyl]-O—[(C₁-C₄)alkyl], —[(C₁-C₄)alkyl]-OC(O)O—[(C₁-C₄)alkyl], —[(C₁-C₄)alkyl]-SC(O)—[(C₁-C₄)alkyl], —[(C₁-C₄)alkyl]-SC(O)—[(C₁-C₄)alkyl]-OH, and phenyl, wherein the remaining variables are as described for Formula I or any one of the third, fourth and sixth to ninth embodiments. Alternatively, as part of a tenth embodiment, R^{1b} and R^{2b} in the compound of Formula I, II, III, or IV, or a pharmaceutically acceptable salt thereof, are each independently selected from hydrogen, [(C₁-C₄)alkyl]-OC(O)—[(C₁-C₄)alkyl], —[(C₁-C₄)alkyl]-OC(O)O—[(C₁-C₄)alkyl]-O—[(C₁-C₄)alkyl], —[(C₁-C₄)alkyl]-OC(O)O—[(C₁-C₄)alkyl], and —[(C₁-C₄)alkyl]-SC(O)—[(C₁-C₄)alkyl], wherein the remaining variables are as described for Formula I or any one of the third, fourth and sixth to ninth embodiments. Alternatively, as part of a tenth embodiment, R^{1b} and R^{2b} in the compound of Formula I, II, III, or IV, or a pharmaceutically acceptable salt thereof, are each —[(C₁-C₄)alkyl]-OC(O)—[(C₁-C₄)alkyl], wherein the remaining variables are as described for Formula I or any one of the third, fourth and sixth to ninth embodiments. Alternatively, as part of a tenth embodiment, R^{1b} and R^{2b} in the compound of Formula I, II, III, or IV, or a pharmaceutically acceptable salt thereof, are each hydrogen, wherein the remaining variables are as described for Formula I or any one of the third, fourth and sixth to ninth embodiments.

In a tenth embodiment, —CR^{1a}R^{2a}P(O)OR^{1b}OR^{2b} in the compound of Formula I, II, III, or IV, or a pharmaceutically acceptable salt thereof, is selected from

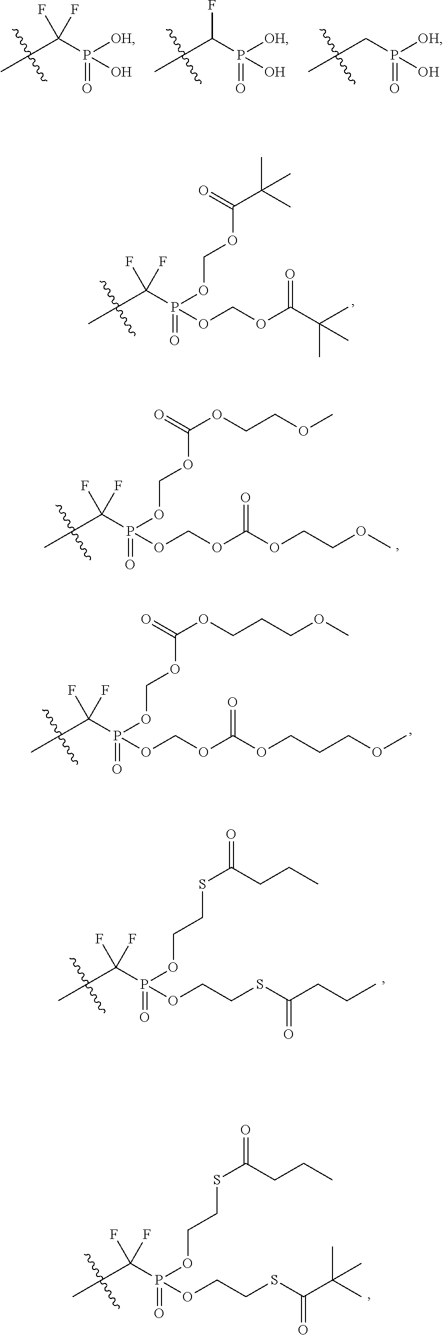

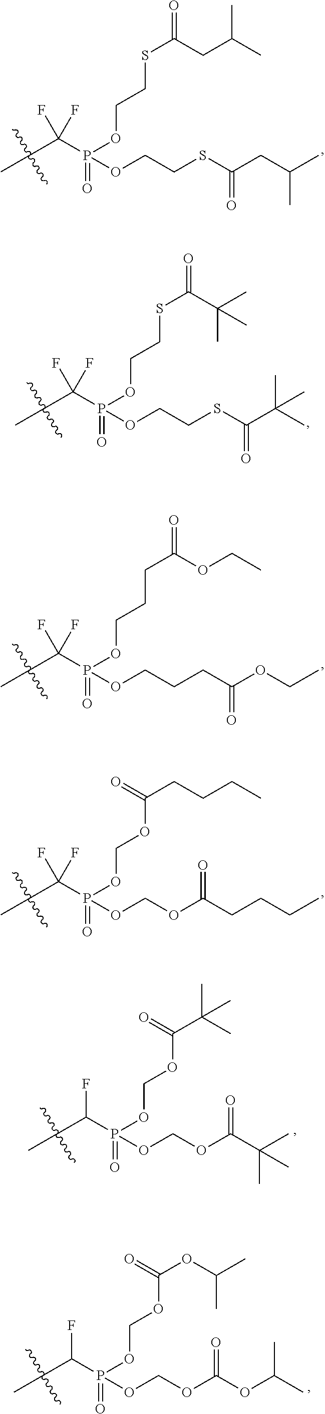 [SMILES: *C(F)(F)P(=O)(OCCSC(=O)CC(C)C)OCCSC(=O)CC(C)C ; *C(F)(F)P(=O)(OCCSC(=O)C(C)(C)C)OCCSC(=O)C(C)(C)C ; *C(F)(F)P(=O)(OCCCC(=O)OCC)OCCCC(=O)OCC ; *C(F)(F)P(=O)(OCOC(=O)CCCC)OCOC(=O)CCCC ; *C(F)P(=O)(OCOC(=O)C(C)(C)C)OCOC(=O)C(C)(C)C ; *C(F)P(=O)(OCOC(=O)OC(C)C)OCOC(=O)OC(C)C]

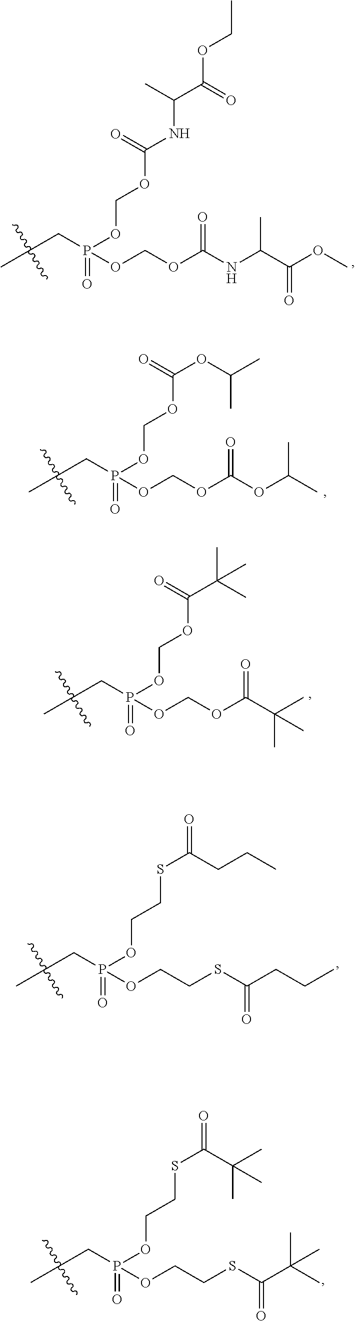 [SMILES: *CP(=O)(OCOC(=O)NC(C)C(=O)OC)OCOC(=O)NC(C)C(=O)OCC ; *CP(=O)(OCOC(=O)OC(C)C)OCOC(=O)OC(C)C ; *CP(=O)(OCOC(=O)C(C)(C)C)OCOC(=O)C(C)(C)C ; *CP(=O)(OCCSC(=O)CCC)OCCSC(=O)CCC ; *CP(=O)(OCCSC(=O)C(C)(C)C)OCCSC(=O)C(C)(C)C]

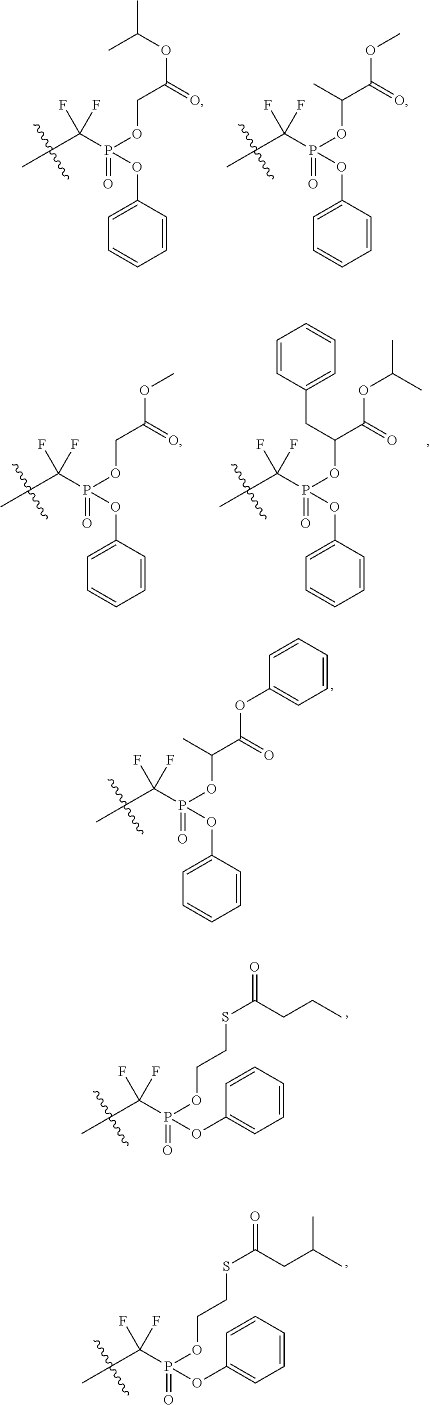

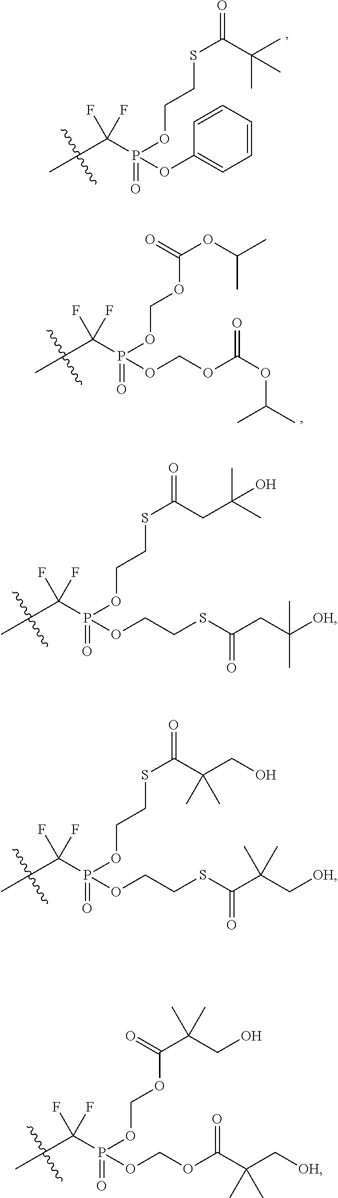

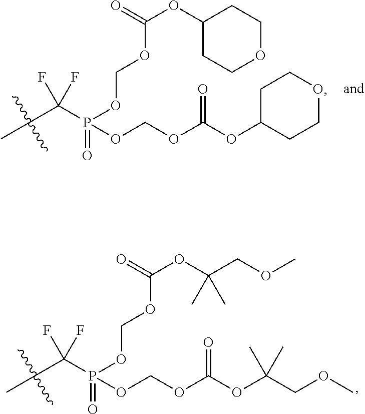 [SMILES: *C(F)(F)P(=O)(OCOC(=O)OC1CCOCC1)OCOC(=O)OC1CCOCC1 ; *C(F)(F)P(=O)(OCOC(=O)OC(C)(C)COC)OCOC(=O)OC(C)(C)COC]

wherein the remaining variables are as described for Formula I or any one of the third, fourth and sixth to eighth embodiments. Alternatively, as part of a tenth embodiment, —CR^{1a}R^{2a}P(O)OR^{1b}OR^{2b} in the compound of Formula I, II, III, or IV, or a pharmaceutically acceptable salt thereof, is

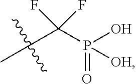 [SMILES: *C(F)(F)P(=O)(O)O]

wherein the remaining variables are as described for Formula I or any one of the third, fourth and sixth to eighth embodiments.

In an eleventh embodiment, -(AA)C(O)OR^{T} in the compound of Formula I, II, III, or IV, or a pharmaceutically acceptable salt thereof, is —C(R′)(R)C(O)R^{T} or —C(R′)(R)CH₂C(O)R^{T}, wherein R′ is hydrogen and R is selected from hydrogen, methyl, —CH₂CH(CH₃)₂, benzyl, and —CH₂CH₂-phenyl, wherein the remaining variables are as described for Formula I or any one of the third, fourth and sixth to eighth embodiments.

In a twelfth embodiment, R^{T} in the compound of Formula I, II, III, or IV, or a pharmaceutically acceptable salt thereof, is selected from (C₁-C₄)alkyl, (C₁-C₄)alkyl-C(O)O—C₁₋₄alkyl, and benzyl, wherein the remaining variables are as described for Formula I or any one of the third, fourth, sixth to eighth, and eleventh embodiments.

In a thirteenth embodiment, —CR^{1a}R^{2a}P(O)[OR^{1b}][NH(AA)C(O)OR^{T}] in the compound of Formula I, II, III, or IV, or a pharmaceutically acceptable salt thereof, is selected from

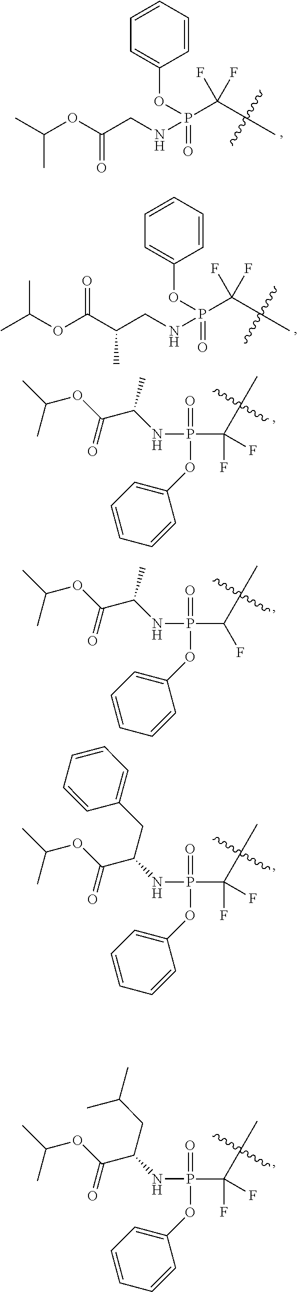

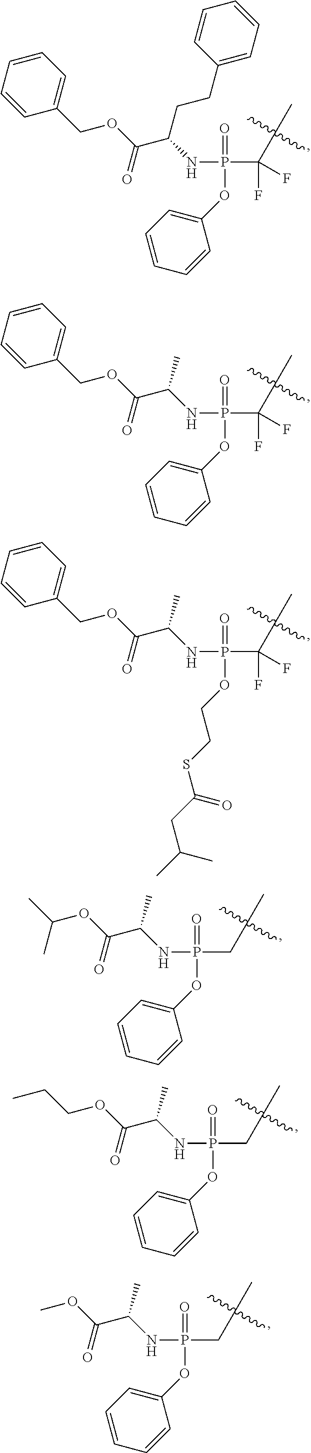

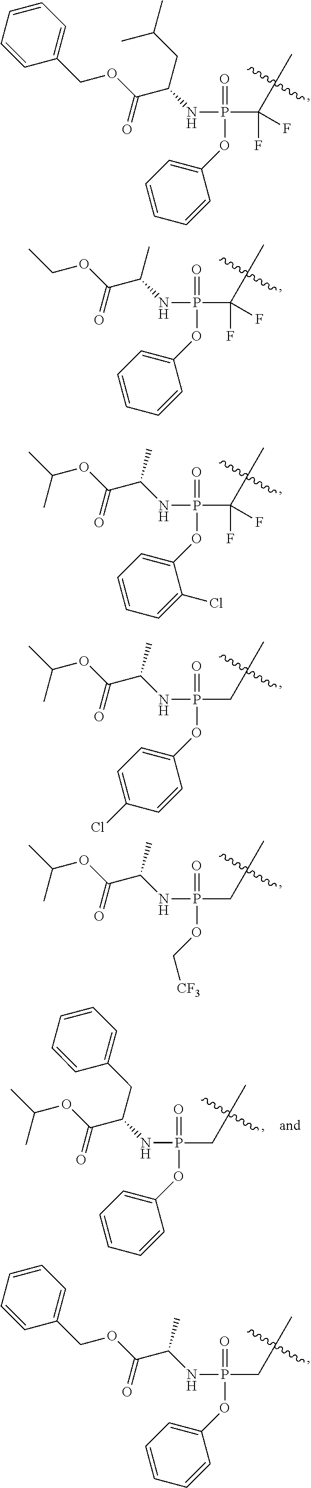

wherein the remaining variables are as described for Formula I or any one of the third, fourth, sixth to eighth, eleventh, and twelfth embodiments. In an alternative thirteenth embodiment, —CR^{1a}R^{2a}P(O)[OR^{1b}][NH(AA)C(O)OR^{T}] in the compound of Formula I, II, III, or IV, or a pharmaceutically acceptable salt thereof, is selected from

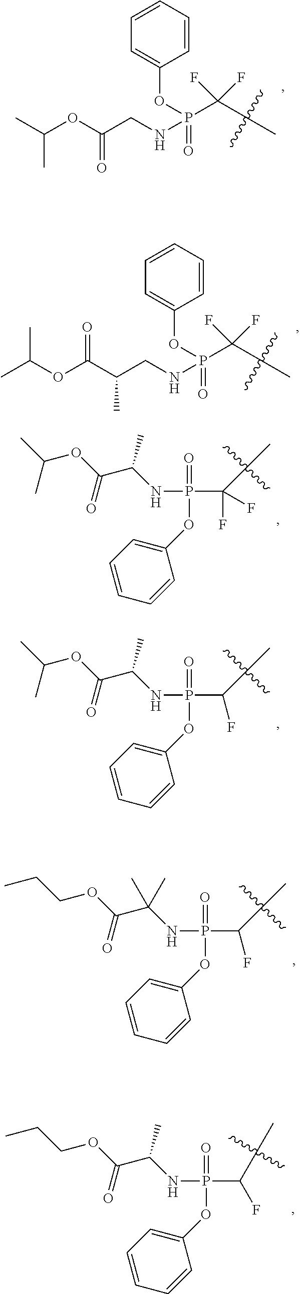

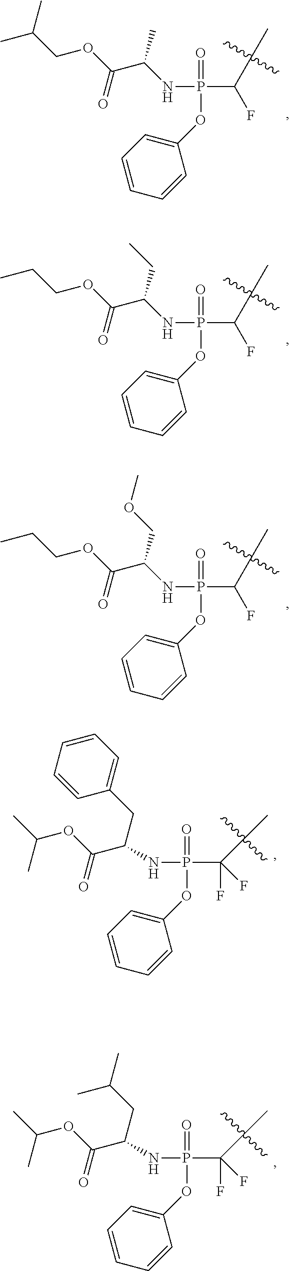 [SMILES: *C(F)P(=O)(N[C@@H](C)C(=O)OCC(C)C)Oc1ccccc1 ; *C(F)P(=O)(N[C@@H](CC)C(=O)OCCC)Oc1ccccc1 ; *C(F)P(=O)(N[C@@H](COC)C(=O)OCCC)Oc1ccccc1 ; *C(F)(F)P(=O)(N[C@@H](Cc1ccccc1)C(=O)OC(C)C)Oc1ccccc1 ; *C(F)(F)P(=O)(N[C@@H](CC(C)C)C(=O)OC(C)C)Oc1ccccc1]

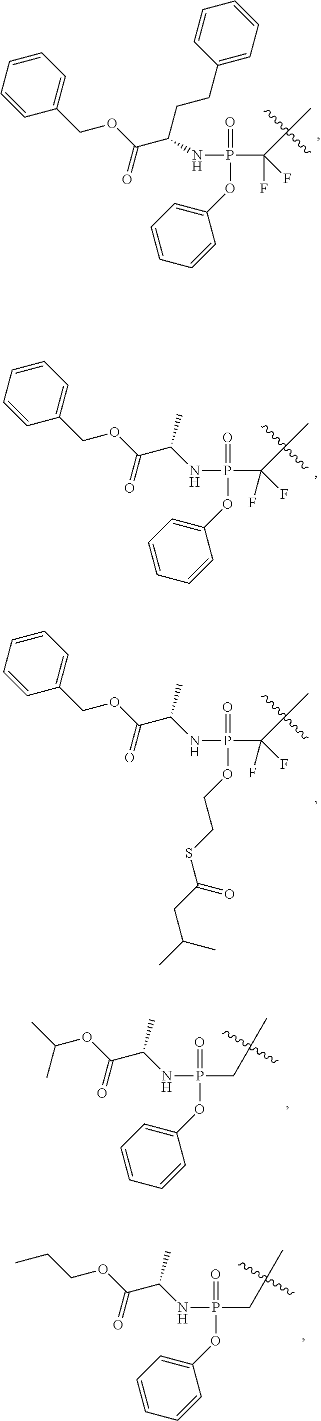 [SMILES: *C(F)(F)P(=O)(N[C@@H](CCc1ccccc1)C(=O)OCc1ccccc1)Oc1ccccc1 ; *C(F)(F)P(=O)(N[C@@H](C)C(=O)OCc1ccccc1)Oc1ccccc1 ; *C(F)(F)P(=O)(N[C@@H](C)C(=O)OCc1ccccc1)OCCSC(=O)CC(C)C ; *CP(=O)(N[C@@H](C)C(=O)OC(C)C)Oc1ccccc1 ; *CP(=O)(N[C@@H](C)C(=O)OCCC)Oc1ccccc1]

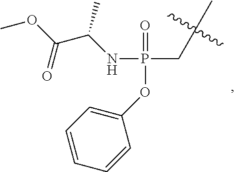 [SMILES: *CP(=O)(N[C@@H](C)C(=O)OC)Oc1ccccc1]

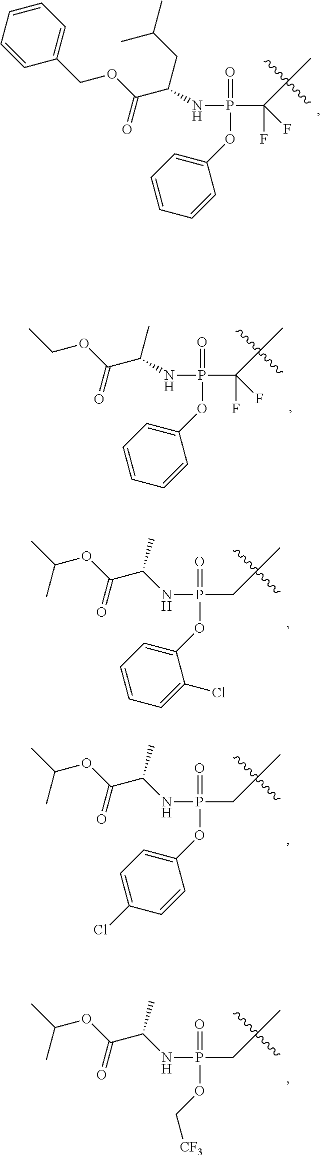

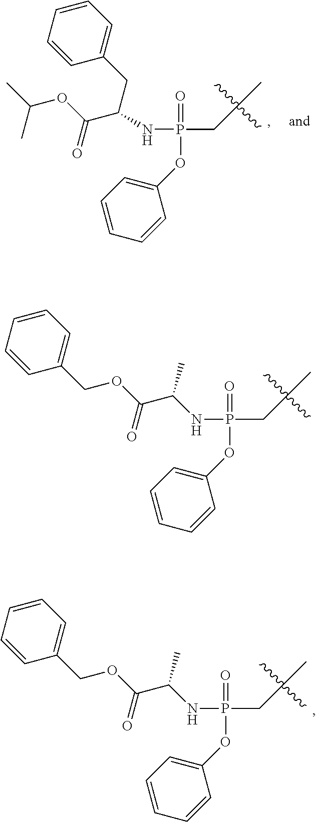 [SMILES: *CP(=O)(N[C@@H](Cc1ccccc1)C(=O)OC(C)C)Oc1ccccc1 ; *CP(=O)(N[C@@H](C)C(=O)OCc1ccccc1)Oc1ccccc1 ; *CP(=O)(N[C@@H](C)C(=O)OCc1ccccc1)Oc1ccccc1]

wherein the remaining variables are as described for Formula I or any one of the third, fourth, sixth to eighth, eleventh, and twelfth embodiments.

In a fourteenth embodiment, R⁶ in the compound of Formula I, II, III, or IV, or a pharmaceutically acceptable salt thereof, is hydrogen, wherein the remaining variables are as described for Formula I or any one of the third to thirteenth embodiments.

In a fifteenth embodiment, R⁷ in the compound of Formula I, II, III, or IV, or a pharmaceutically acceptable salt thereof, is selected from (C₁-C₄)alkyl, phenyl, and 4- to 6-membered monocyclic heterocyclyl, wherein said (C₁-C₄)alkyl is optionally substituted with, as valency permits, 1 to 3 groups selected from R^{Y} and said phenyl and 4- to 6-membered monocyclic heterocyclyl are each optionally substituted with, as valency permits, 1 to 3 groups selected from R^{Z}, wherein the remaining variables are as described for Formula I or any one of the third to fourteenth embodiments. Alternatively, as part of a fifteenth embodiment, R⁷ in the compound of Formula I, II, III, or IV, or a pharmaceutically acceptable salt thereof, is selected from (C₁-C₄)alkyl, phenyl, pyrrolidinyl, and azetidinyl, wherein said (C₁-C₄)alkyl is optionally substituted with, as valency permits, 1 to 3 groups selected from R^{Y} and said phenyl, pyrrolidinyl, and azetidinyl are each optionally substituted with, as valency permits, 1 to 3 groups selected from R^{Z}, wherein the remaining variables are as described for Formula I or any one of the third to fourteenth embodiments.

In a sixteenth embodiment, R^{Z} in the compound of Formula I, II, III, or IV, or a pharmaceutically acceptable salt thereof, is selected from halo, —(C₁-C₄)alkylC(O)NR^{c}R^{d}, hydroxyl, phenyl, 4- to 6-membered heterocyclyl, 5- to 6-membered monocyclic heteroaryl, —C(O)NR^{c}R^{d}, and —C(O)R^{g}, wherein said phenyl is optionally substituted with, as valency permits 1 to 3 groups selected from halo, (C₁-C₄)alkyl, halo(C₁-C₄)alkyl, (C₁-C₄)alkoxy, and halo(C₁-C₄)alkoxy, wherein the remaining variables are as described for Formula I or any one of the third to fifteenth embodiments. Alternatively, as part of a sixteenth embodiment, R^{Z} in the compound of Formula I, II, III, or IV, or a pharmaceutically acceptable salt thereof, is selected from halo, —(C₁-C₄)alkylC(O)NR^{c}R^{d}, hydroxyl, phenyl, tetrahydropyran, tetrahydrofuran, oxetanyl, pyridinyl, pyrazolyl, pyridazinyl, —C(O)NR^{c}R^{d} and —C(O)R^{g}, wherein the remaining variables are as described for Formula I or any one of the third to fifteenth embodiments. Alternatively, as part of a sixteenth embodiment, R^{Z} in the compound of Formula I, II, III, or IV, or a pharmaceutically acceptable salt thereof, is selected from halo, —(C₁-C₄)alkylC(O)NR^{c}R^{d}, hydroxyl, phenyl, tetrahydropyran, tetrahydrofuran, oxetanyl, pyridinyl, pyrimidinyl, imidazoyl, triazoyl, pyrazolyl, pyridazinyl, —C(O)NR^{c}R^{d} and —C(O)R^{g}, wherein the pyridinyl, imidazoyl, and triazoyl are optionally substituted with one or two groups selected from halo and methyl, and wherein the remaining variables are as described for Formula I or any one of the third to fifteenth embodiments.

In a seventeenth embodiment, R^{Y} in the compound of Formula I, II, III, or IV, or a pharmaceutically acceptable salt thereof, is selected from hydroxyl and 5- to 10-membered monocyclic or bicyclic heteroaryl, wherein said 5- to 10-membered monocyclic or bicyclic is optionally substituted with, as valency permits, 1 to 3 groups selected from R^{X}, wherein the remaining variables are as described for Formula I or any one of the third to sixteenth embodiments. Alternatively, as part of a seventeenth embodiment, R^{Y} in the compound of Formula I, II, III, or IV, or a pharmaceutically acceptable salt thereof, is selected from hydroxyl, pyridinyl, and pyrrolopyridinyl, wherein the remaining variables are as described for Formula I or any one of the third to sixteenth embodiments.

In an eighteenth embodiment, R^{c} and R^{d} in the compound of Formula I, II, III, or IV, or a pharmaceutically acceptable salt thereof, are each hydrogen, wherein the remaining variables are as described for Formula I or any one of the third to seventeenth embodiments.

In a nineteenth embodiment, R^{g} in the compound of Formula I, II, III, or IV, or a pharmaceutically acceptable salt thereof, is —(C₁-C₄)alkyl, wherein the remaining variables are as described for Formula I or any one of the third to eighteenth embodiments.

In a twentieth embodiment, R⁶ and R⁷ in the compound of Formula I, II, III, or IV, or a pharmaceutically acceptable salt thereof, together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, 1 to 3 groups selected from R^{Q}, wherein the remaining variables are as described for Formula I or any one of the third to nineteenth embodiments. Alternatively, as part of a twentieth embodiment, R⁶ and R⁷ in the compound of Formula I, II, III, or IV, or a pharmaceutically acceptable salt thereof, together with the nitrogen atom to which they are attached form azetidinyl, 2,5-diazaspiro[3.4]octanyl, pyrrolidinyl, 2,6-diazaspiro[3.3]heptanyl, piperazinyl, spiro[indoline-3,3′-pyrrolidine]yl, 6′,7′-dihydrospiro[azetidine-3,5′-pyrrolo[1,2-a]imidazole]yl, each of which being optionally substituted with, as valency permits, 1 to 3 groups selected from R^{Q}, wherein the remaining variables are as described for Formula I or any one of the third to nineteenth embodiments. Alternatively, as part of a twentieth embodiment, R⁶ and R⁷ in the compound of Formula I, II, III, or IV, or a pharmaceutically acceptable salt thereof, together with the nitrogen atom to which they are attached form azetidinyl, 2,5-diazaspiro[3.4]octanyl, pyrrolidinyl, 2,6-diazaspiro[3.3]heptanyl, 2,6-diazabicyclo[3.2.0]heptanyl, piperazinyl, spiro[indoline-3,3′-pyrrolidine]yl, 6′,7′-dihydrospiro[azetidine-3,5′-pyrrolo[1,2-a]imidazole]yl, 3,4-dihydro-2H-benzo[b][1,4]oxazinyl, 3,4-dihydro-2H-pyrido[3,2-b][1,4]oxazine, 2,3-dihydro-1H-pyrido[2,3-b][1,4]oxazine, 2,3,4,5-tetrahydrobenzo[b][1,4]oxazepinyl, 1,2,3,4-tetrahydroquinoxalinyl, 1-azaspiro[3.5]nonanyl, 4-azaspiro[2.4]heptanyl, each of which being optionally substituted with, as valency permits, 1 to 3 groups selected from R^{Q}, wherein the remaining variables are as described for Formula I or any one of the third to nineteenth embodiments.

In a twenty-first embodiment, R^{Q} in the compound of Formula I, II, III, or IV, or a pharmaceutically acceptable salt thereof, is selected from halo, (C₁-C₄)alkyl, (C₁-C₄)alkoxy, cyano, phenyl, hydroxyl, 4- to 6-membered heterocyclyl, 5- to 10-membered monocyclic or bicyclic heteroaryl, oxo, and, —C(O)R^{g}, wherein said (C₁-C₄)alkyl is optionally substituted with, as valency permits, 1 to 3 groups selected from R^{M}, and wherein said phenyl, 5- to 10-membered monocyclic or bicyclic heteroaryl, and 4- to 6-membered heterocyclyl are each optionally and independently substituted with, as valency permits, 1 to 3 groups selected from R^{F}, wherein the remaining variables are as described for Formula I or any one of the third to thirteenth and twentieth embodiments. Alternatively, as part of a twenty-first embodiment, R^{Q} in the compound of Formula I, II, III, or IV, or a pharmaceutically acceptable salt thereof, is selected from halo, (C₁-C₄)alkyl, —OR^{e}, cyano, phenyl, hydroxyl, 4- to 6-membered heterocyclyl, 5- to 10-membered monocyclic or bicyclic heteroaryl, oxo, and, —C(O)R^{g}, wherein said (C₁-C₄)alkyl is optionally substituted with, as valency permits, 1 to 3 groups selected from R^{M}, and wherein said phenyl, 5- to 10-membered monocyclic or bicyclic heteroaryl, and 4- to 6-membered heterocyclyl are each optionally and independently substituted with, as valency permits, 1 to 3 groups selected from R^{F}, and wherein R^{e} is (C₁-C₄)alkyl or 5- to 6-membered heteroaryl, wherein the remaining variables are as described for Formula I or any one of the third to thirteenth and twentieth embodiments. Alternatively, as part of a twenty-first embodiment, R^{Q} in the compound of Formula I, II, III, or IV, or a pharmaceutically acceptable salt thereof, is selected from halo, (C₁-C₄)alkyl, (C₁-C₄)alkoxy, cyano, phenyl, hydroxyl, morpholinyl, tetrahydropyranyl, thiomorpholinyl, piperidinyl, oxatanyl, pyrazolyl, pyridinyl, tetrazolyl, imidazolyl, pyrazinyl, oxadiazolyl, triazolyl, pyrimidinyl, benzoimidazolyl, 2,4,5,6-tetrahydrocyclopenta[c]pyrazolyl, oxo, and, —C(O)R^{g}, wherein said (C₁-C₄)alkyl is optionally substituted with, as valency permits, 1 to 3 groups selected from R^{M}, and wherein said morpholinyl, tetrahydropyranyl, thiomorpholinyl, piperidinyl, oxatanyl, pyrazolyl, pyridinyl, tetrazolyl, imidazolyl, pyrazinyl, oxadiazolyl, triazolyl, pyrimidinyl, benzoimidazolyl, and 2,4,5,6-tetrahydrocyclopenta[c]pyrazolyl are each optionally and independently substituted with, as valency permits, 1 to 3 groups selected from R^{F}, wherein the remaining variables are as described for Formula I or any one of the third to thirteenth and twentieth embodiments. Alternatively, as part of a twenty-first embodiment, R^{Q} in the compound of Formula I, II, III, or IV, or a pharmaceutically acceptable salt thereof, is selected from halo, (C₁-C₄)alkyl, —OR^{e}, cyano, phenyl, hydroxyl, morpholinyl, tetrahydropyranyl, thiomorpholinyl, piperidinyl, oxatanyl, pyrazolyl, pyridinyl, tetrazolyl, imidazolyl, pyrazinyl, isoxazoyl, oxazoyl, oxadiazolyl, triazolyl, pyrimidinyl, benzoimidazolyl, 1H-pyrrolo[3,2-c]pyridine, 2,4,5,6-tetrahydrocyclopenta[c]pyrazolyl, oxo, and, —C(O)R^{g}, wherein said (C₁-C₄)alkyl is optionally substituted with, as valency permits, 1 to 3 groups selected from R^{M}, and wherein said morpholinyl, tetrahydropyranyl, thiomorpholinyl, piperidinyl, oxatanyl, pyrazolyl, pyridinyl, tetrazolyl, imidazolyl, pyrazinyl, isoxazoyl, oxazoyl, oxadiazolyl, triazolyl, pyrimidinyl, benzoimidazolyl, 1H-pyrrolo[3,2-c]pyridine, and 2,4,5,6-tetrahydrocyclopenta[c]pyrazolyl are each optionally and independently substituted with, as valency permits, 1 to 3 groups selected from R^{F}, wherein R^{e} is (C₁-C₄)alkyl, pyridinyl, pyrazinyl, pyrimidinyl, pyrazole, and wherein the pyrazoyl represented by R^{e} is optionally substituted with (C₁-C₄)alkyl, and wherein the remaining variables are as described for Formula I or any one of the third to thirteenth and twentieth embodiments.

In a twenty-second embodiment, R^{g} in the compound of Formula I, II, III, or IV, or a pharmaceutically acceptable salt thereof, is selected from, as valency permits, (C₁-C₄)alkyl, 4- to 6-membered heterocyclyl, and 5- to 6-membered heteroaryl, wherein said (C₁-C₄)alkyl is optionally substituted with, as valency permits, 1 to 3 groups selected from R^{J}, and said 4- to 6-membered heterocyclyl, and 5- to 6-membered heteroaryl are each independently optionally substituted with, as valency permits, 1 to 3 groups selected from (C₁-C₄)alkyl, (C₁-C₄)alkoxy, benzyl, and hydroxyl, wherein the remaining variables are as described for Formula I or any one of the third to twenty-first embodiments.

In a twenty-third embodiment, R^{J} in the compound of Formula I, II, III, or IV, or a pharmaceutically acceptable salt thereof, is phenyl, wherein the remaining variables are as described for Formula I or any one of the third to twenty-second embodiments.

In a twenty-fourth embodiment, R^{g} in the compound of Formula I, II, III, or IV, or a pharmaceutically acceptable salt thereof, is selected from, as valency permits, (C₁-C₄)alkyl, morpholinyl, azetidinyl, tetrahydropyranyl, oxatanyl, pyrrolidinyl, and pyrazolyl, wherein said (C₁-C₄)alkyl is optionally substituted with, as valency permits, 1 to 3 groups selected from R^{J}, and said morpholinyl, azetidinyl, tetrahydropyranyl, oxatanyl, pyrrolidinyl, and pyrazolyl are each independently optionally substituted with, as valency permits, 1 to 3 groups selected from (C₁-C₄)alkyl, (C₁-C₄)alkoxy, benzyl, and hydroxyl, wherein the remaining variables are as described for Formula I or any one of the third to twenty-third embodiments.

In a twenty-fifth embodiment, R^{F} in the compound of Formula I, II, III, or IV, or a pharmaceutically acceptable salt thereof, is selected from cyano, (C₁-C₄)alkyl, hydroxyl, and oxo, wherein the remaining variables are as described for Formula I or any one of the third to twenty-fourth embodiments. Alternatively, as part of the twenty-fifth embodiment, R^{F} in the compound of Formula I, II, III, or IV, or a pharmaceutically acceptable salt thereof, is selected from halo, cyano, (C₁-C₄)alkyl, (C₁-C₄)haloalkyl, (C₁-C₄)alkoxy, hydroxyl, —N[(C₁-C₄)alkyl]₂, morpholinyl, piperazinyl, azetidinyl, pyrrolidinyl, and oxo, wherein said piperazinyl, pyrrolidinyl, and azetidinyl are each optionally substituted with 1 or 2 groups selected from cyano, halo, (C₁-C₄)alkyl, (C₁-C₄)alkoxy, and (C₁-C₄)alkyl(C₁-C₄)alkoxy, wherein the remaining variables are as described for Formula I or any one of the third to twenty-fourth embodiments.

In a twenty-sixth embodiment, R^{M} in the compound of Formula I, II, III, or IV, or a pharmaceutically acceptable salt thereof, is selected from halo, (C₁-C₄)alkoxy, —S(O)₂R^{f}, and —S(O)═NH(C₁-C₄)alkyl, wherein the remaining variables are as described for Formula I or any one of the third to twenty-fifth embodiments. Alternatively, as part of the twenty-sixth embodiment, R^{M} in the compound of Formula I, II, III, or IV, or a pharmaceutically acceptable salt thereof, is selected from halo, hydroxy, (C₁-C₄)alkoxy, —S(O)₂R^{f}, —S(O)═NH(C₁-C₄)alkyl, pyridinyl, pyrazoyl, and phenyl optionally substituted with 1 or 2 halo, wherein the remaining variables are as described for Formula I or any one of the third to twenty-fifth embodiments.

In a twenty-seventh embodiment, R^{f} in the compound of Formula I, II, III, or IV, or a pharmaceutically acceptable salt thereof, is (C₁-C₄)alkyl, and wherein said (C₁-C₄)alkyl is optionally substituted with, as valency permits, 1 to 3 halo, wherein the remaining variables are as described for Formula I or any one of the third to twenty-sixth embodiments.

Compounds having the Formula I are further disclosed in the Exemplification and are included in the present disclosure. Pharmaceutically acceptable salts thereof as well as the neutral forms are included.

### 4. Uses, Formulation and Administration

The compounds and compositions described herein are generally useful for modulating the activity of STAT proteins, in particular STAT3 and/or STAT6. In some aspects, the compounds, pharmaceutical acceptable salts, and pharmaceutical compositions described herein inhibit the activity STAT3 and/or STAT6.

In some aspects, the compounds and pharmaceutical compositions described herein are useful in a condition responsive to the modulation of STAT3 and/or STAT6. Thus, provided herein are methods of treating a condition responsive to the modulation (e.g., inhibition) of STAT3 and/or STAT6 in a subject, comprising administering to a subject in need thereof, a therapeutically effective amount of a compound described herein, or a pharmaceutically acceptable salt thereof, or a pharmaceutical composition comprising a disclosed compound or pharmaceutically acceptable salt thereof.

Also provided is the use of a compound described herein, or a pharmaceutically acceptable salt thereof, or a pharmaceutical composition comprising a disclosed compound or pharmaceutically acceptable salt thereof, for the manufacture of a medicament for treating a condition responsive to the modulation (e.g., inhibition) of STAT3 and/or STAT6. Also provided is a compound described herein, or a pharmaceutically acceptable salt thereof, or a pharmaceutical composition comprising a disclosed compound or pharmaceutically acceptable salt thereof, for use in treating a condition responsive to the modulation (e.g., inhibition) of STAT3 and/or STAT6.

In one aspect, the condition responsive to the modulation (e.g., inhibition) of STAT3 and/or STAT6 include, but are not limited to, cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, or a CNS disorder.

In another aspect, the condition responsive to the modulation (e.g., inhibition) of STAT3 and/or STAT6 include, but are not limited to, cancer (see, e.g., Turkson & Jove, Oncogene 2000, 19:6613-6626), diabetes (see. e.g., Gurzov et al., FEBS 2016, 283:3002), cardiovascular disease (see, e.g., Grote et al., Vasc. Pharmacol. 2005, 43:2005), viral disease (see, e.g., Gao et al., J Hepatol. 2012, 57(2):430), autoimmune diseases such as lupus (see, e.g., Goropevsek et al., Clin. Rev. Alleg. & Immun. 2017, 52(2):164), and rheumatoid arthritis (see, e.g., Walker & Smith, J. Rheumat. 2005, 32(9): 1650), autoinflammatory syndromes (see, e.g., Rauch et al., Jak-Stat 2013, 2(1):e23820), atherosclerosis (see, e.g., Ortiz-Munoz et al., Arterio., Thromho., Vase. Bio. 2009, 29:525), psoriasis (see, e.g., Andres et al., Exp. Derm. 2013, 22(5):323), allergic disorders (see, e.g., Oh et al., Eur. Respir. Rev. 2019, 19(115):46), inflammatory bowel disease (see. e.g., Sugimoto, World J Gastroenterol. 2008, 14(33):5110), inflammation (see, e.g., Tamiya et al., Arierio. Thrombo., Vasc. Bio. 2011, 31:980), acute and chronic gout and gouty arthritis, neurological disorders (see, e.g., Campbell, Brain Res. Rev. 2005, 48(2): 166), metabolic syndrome, immunodeficiency disorders such as AIDS and HIV (see, e.g., O'Shea et al., N. Engl. J. Med. 2013, 368:161), destructive bone disorders (see, e.g., Jatiani et al., Genes & Can. 2011, 1(10):979), osteoarthritis, proliferative disorders, Waldenstrom's Macroglobulinemia (see, e.g., Hodge et al., Blood 2014, 123(7):1055) infectious diseases, conditions associated with cell death, pathologic immune conditions involving T cell activation, and CNS disorders.

Proliferative disorders, include, but are not limited to a benign or malignant tumor, solid tumor, liquid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins and Non-Hodgkins, a mammary carcinoma, follicular carcinoma, undifferentiated carcinoma, papillary carcinoma, seminoma, melanoma, an IL-I driven disorder, an MyD88 driven disorder, Smoldering of indolent multiple myeloma, or hematological malignancies (including leukemia, diffuse large B-cell lymphoma (DLBCL), ABC DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenstrom's macroglobulinemia (WM), splenic marginal zone lymphoma, multiple myeloma, plasmacytoma, intravascular large B-cell lymphoma).

In some embodiments, the cancer to be treated is selected from glioma, breast cancer, prostate cancer, head and neck squamous cell carcinoma, skin melanomas, ovarian cancer, malignant peripheral nerve sheath tumors (MPNST), and pancreatic cancer. In other embodiments, the cancer to be treated is cancer selected from glioma, breast cancer, prostate cancer, head and neck squamous cell carcinoma, skin melanomas, ovarian cancer, malignant peripheral nerve shealth tumors (MPNST), pancreatic cancer, non-small cell lung cancer (NSCLC) including EGFR-mutant NSCLC, urothelial cancer, liver cancer, bile duct cancer, kidney cancer, colon cancer, esophageal cancer, gastric cancer, gastrointestinal stromal tumors, and hematological malignancies include lymphomas, leukemias, myelomas, myeloproliferative neoplasms and myelodysplastic syndromes. In other embodiments, the cancer is selected from solid tumors (e.g., prostate cancer, renal cancer, hepatic cancer, pancreatic cancer, gastric cancer, breast cancer, lung cancer, cancers of the head and neck, thyroid cancer, glioblastoma, Kaposi's sarcoma, Castleman's disease, uterine leiomyosarcoma, melanoma etc.), hematological cancers (e.g., lymphoma, leukemia Such as acute lymphoblastic leukemia (ALL), acute myelogenous leukemia (AML) or multiple myeloma), and skin cancer such as cutaneous T-cell lymphoma (CTCL) and cutaneous B-cell lymphoma. Example CTCLs include Sezary syndrome and mycosis fungoides.

Compounds, salts, and compositions described herein are also useful in the treatment of inflammatory or obstructive airways diseases, resulting, for example, in reduction of tissue damage, airways inflammation, bronchial hyperreactivity, remodeling or disease progression. Inflammatory or obstructive airways diseases include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics.

Compounds, salts, and compositions described herein are also useful in the treatment of heteroimmune diseases including, but are not limited to, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

Compounds, salts, and compositions described herein are also useful in the treatment of other inflammatory or obstructive airways diseases and conditions to which the present invention is applicable and include acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. Compounds, salts, and compositions described herein are also useful in the treatment of bronchitis including, but not limited to, acute, arachidic, catarrhal, croupus, chronic or phthinoid bronchitis. Compounds, salts, and compositions described herein are also useful in the treatment of pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

Compounds, salts, and compositions described herein are also useful in the treatment of inflammatory or allergic conditions of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

Compounds, salts, and compositions described herein are also useful in the treatment of other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospiriosis renal disease, glaucoma, retinal disease, ageing, headache, pain, complex regional pain syndrome, cardiac hypertrophy, musclewasting, catabolic disorders, obesity, fetal growth retardation, hyperchlolesterolemia, heart disease, chronic heart failure, mesothelioma, anhidrotic ectodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acidinduced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, encephalomyelitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments, cardiovascular diseases which can be treated according to the present methods include, but are not limited to, restenosis, cardiomegaly, atherosclerosis, myocardial infarction, ischemic stroke, congestive heart failure, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, and deep venous thrombosis.

In some embodiments, the neurodegenerative disease which can be treated according to the present methods include, but are not limited to, Alzheimer's disease, Parkinson's disease, amyotrophic lateral sclerosis, Huntington's disease, cerebral ischemia, and neurodegenerative disease caused by traumatic injury, glutamate neurotoxicity, hypoxia, epilepsy, treatment of diabetes, metabolic syndrome, obesity, organ transplantation and graft versus host disease.

In certain aspects, a pharmaceutical composition described herein is formulated for administration to a patient in need of such composition. Pharmaceutical compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the pharmaceutical compositions described herein may be aqueous or oleaginous suspension. These suspensions may be formulated according to techniques known in the art using suitable dispersing or wetting agents and suspending agents.

In some aspects, the pharmaceutical compositions are administered orally.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the pharmaceutical composition.

### EXEMPLIFICATION

### Preparation of Compounds

The compounds claimed herein were prepared following the procedures outlined in the following schemes. Compound names were generated using the software built into ChemDraw. To the extent that there are discrepancies between the name of a compound and its depicted structure, the depicted chemical structure is to be taken as the appropriate compound.

### Synthesis of Cores

Synthesis of (3S,6S,7aS,8aR,9aR)-6-((tert-butoxycarbonyl)amino)-5-oxodecahydro-1H-cyclopropa[d]pyrrolo[1,2-a]azocine-3-carboxylic acid and (3S,6S,7aR,8aS,9aR)-6-((tert-butoxycarbonyl)amino)-5-oxodecahydro-1H-cyclopropa[d]pyrrolo[1,2-a]azocine-3-carboxylic acid

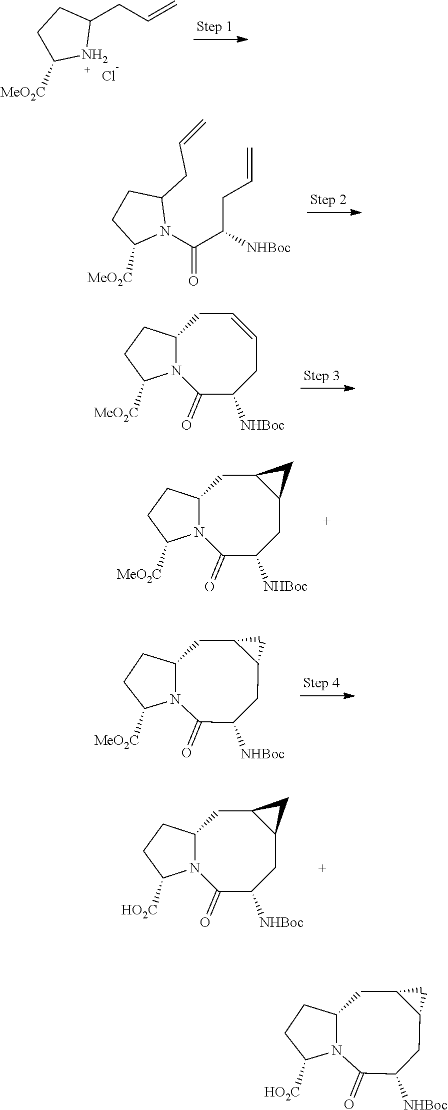

### Step 1: Preparation of methyl (2S)-5-allyl-1-((S)-2-((tert-butoxycarbonyl)amino)pent-4-enoyl)pyrrolidine-2-carboxylate

To a cooled (0° C.) solution of methyl (2S)-5-allylpyrrolidine-2-carboxylate hydrochloride (200 g, 972 mmol, 1.00 eq) and (S)-2-((tert-butoxycarbonyl)amino)pent-4-enoic acid (209 g, 972 mmol, 1.00 eq) in CH₂Cl₂ (1.60 L) was added Et₃N (406 mL, 2.92 mol, 3.00 eq) and 2-chloro-1-methylpyridinium iodide (CMPI) (273 g, 1.07 mol, 1.10 eq). The solution was warmed to 25° C. and stirred for 1 h. The mixture was poured into water (5.0 L), extracted with CH₂Cl₂ (2.00 L×3). The combined organic layers were washed with brine (2.0 L), dried over Na₂SO₄, filtered, and concentrated under reduced pressure. Six individual batches of equal scale were performed in parallel and combined during work up. The resulting residue was purified by column chromatography (petroleum ether:EtOAc=100:1 to 10:1) to give methyl (2S)-5-allyl-1-((S)-2-((tert-butoxycarbonyl)amino)pent-4-enoyl)pyrrolidine-2-carboxylate (1.18 kg, 3.22 mol, 55.2% yield) as yellow oil. ¹H NMR (400 MHz, CDCl₃) δ 5.84-5.78 (m, 2H), 5.17-4.99 (m, 5H), 4.50-4.34 (m, 3H), 3.76-3.70 (m, 3H), 2.50-2.15 (m, 6H), 1.96-1.91 (m, 2H), 1.41 (s, 9H).

### Step 2: Preparation of methyl (3S,6S,10aR,Z)-6-((tert-butoxycarbonyl)amino)-5-oxo-1,2,3,5,6,7,10,10a-octahydropyrrolo[1,2-a]azocine-3-carboxylate

To a solution of methyl (2S)-5-allyl-1-((S)-2-((tert-butoxycarbonyl)amino)pent-4-enoyl)pyrrolidine-2-carboxylate (200 g, 546 mmol, 1.00 eq) in CH₂Cl₂ (2.00 L) was added 1^{st} generation Grubbs catalyst (44.9 g, 54.6 mmol, 0.10 eq) at 25° C. The solution was subsequently heated to 50° C. and stirred for 36 h. Six individual batches of equal scale were performed in parallel and combined during work up. The combined reaction mixtures were concentrated to give a residue. The residue was purified by column chromatography (petroleum ether:EtOAc=100:1 to 0:1) twice to give a crude product. The crude product was triturated with petroleum ether (2.00 L) for 12 h and filtered. The filter cake was dried under reduced pressure to give methyl (3S,6S,10aR,Z)-6-((tert-butoxycarbonyl)amino)-5-oxo-1,2,3,5,6,7,10,10a-octahydropyrrolo[1,2-a]azocine-3-carboxylate (510 g, 1.40 mol, 51.3% yield, 92.9% purity) as a solid. LCMS (ESI) m/z=361.2 [M+Na]⁺; ¹H NMR (400 MHz, CDCl₃) δ 5.82-5.80 (m, 1H), 5.73-5.71 (m, 1H), 5.58-5.56 (m, 1H), 4.88-4.85 (m, 1H), 4.52-4.49 (m, 1H), 4.16-4.14 (m, 1H), 3.71 (s, 3H), 2.81-2.74 (m, 2H), 2.45-2.38 (m, 1H), 2.33-2.24 (m, 1H), 2.14-2.05 (m, 2H), 1.98-1.93 (m, 2H), 1.43 (s, 9H).

Step 3: Preparation of methyl (3S,6S,7aS,8aR,9aR)-6-((tert-butoxycarbonyl)amino)-5-oxodecahydro-1H-cyclopropa[d]pyrrolo[1,2-a]azocine-3-carboxylate and methyl (3S,6S,7aR,8aS,9aR)-6-((tert-butoxycarbonyl)amino)-5-oxodecahydro-1H-cyclopropa[d]pyrrolo[1,2-a]azocine-3-carboxylate

To a cooled (0° C.) solution of 1-methyl-1-nitrosourea (50.0 g, 485 mmol, 1.00 eq) in Et₂O (1.50 L) was slowly added in a dropwise fashion a solution of KOH (144 g, 2.57 mol, 5.30 eq) in H₂O (150 mL). The mixture was stirred at 0° C. for 30 mins, at which point the organic layer turned yellow showed the reaction was finished. The aqueous layer was reaction mixture was separated and the yellow Et₂O layer was used to next step without further manipulation. [Diazomethane (20.0 g, 475 mmol, 98.0% yield) in Et₂O (1.50 L) were obtained].

To a (0° C.) solution of methyl (3S,6S,10aR,Z)-6-((tert-butoxycarbonyl)amino)-5-oxo-1,2,3,5,6,7,10,10a-octahydropyrrolo[1,2-a]azocine-3-carboxylate (10.0 g, 29.5 mmol, 1.00 eq) in THF (320 mL) was carefully transferred approximately 85% of a solution of diazomethane (20.0 g, 476 mmol, 16.1 eq) in Et₂O (1.50 L). To the resulting mixture was slowly added ˜50% of a solution of Pd(OAc)₂ (1.99 g, 8.87 mmol, 0.30 eq) in THF (60.0 mL). The reaction mixture was stirred at 0° C. for 2 mins, followed by successive addition of the remainder ethereal diazomethane solution and the palladium solution. The reaction was stirred at 0° C. for 20 mins, then the ice-water bath was removed, and the reaction was allowed to warm to 20° C. After stirring for 60 mins. The solvent was removed by a nitrogen flow. The resulting dry reaction mixture was diluted with EtOAc and filtered through Celite. The residue (total: 37.0 g) were purified by column: Phenomenex luma C18 250×80 mm×10 um; mobile phase: [water (0.225% FA)-ACN]; B %: 45%-70%, 21 mins. The appropriate peak corresponding to product were collected. The respective samples were adjusted to pH˜8 with aqueous saturated NaHCO₃ solution and concentrated respectively. The respective aqueous layers were extracted with EtOAc (2×1.00 L), washed with brine (1.00 L), dried over Na₂SO₄, filtered, and concentrated to yield methyl (3S,6S,7aS,8aR,9aR)-6-((tert-butoxycarbonyl)amino)-5-oxodecahydro-1H-cyclopropa[d]pyrrolo[1,2-a]azocine-3-carboxylate (12.13 g, 33.5 mmol, 31.5% yield, 97.3% purity) as a white solid and methyl (3S,6S,7aR,8aS,9aR)-6-((tert-butoxycarbonyl)amino)-5-oxodecahydro-1H-cyclopropa[d]pyrrolo[1,2-a]azocine-3-carboxylate (9.12 g, 25.5 mmol, 24.0% yield, 98.6% purity) as a white solid. Additionally, methyl (3S,6S,10aR,Z)-6-((tert-butoxycarbonyl)amino)-5-oxo-1,2,3,5,6,7,10,10a-octahydropyrrolo[1,2-a]azocine-3-carboxylate (7.60 g, 99.0% purity) was recovered as a yellow oil. Methyl (3S,6S,7aS,8aR,9aR)-6-((tert-butoxycarbonyl)amino)-5-oxodecahydro-1H-cyclopropa[d]pyrrolo[1,2-a]azocine-3-carboxylate: LCMS (ESI) m/z=353.0 [M+H]⁺; ¹H NMR (400 MHz, DMSO-d₆) δ 6.81 (d, J=7.6 Hz, 0.8H), 6.50 (d, J=3.6 Hz, 0.2H), 4.44-4.27 (m, 2H), 4.08-3.99 (m, 1H), 3.58 (s, 3H), 2.19-2.07 (m, 4H), 1.88-1.86 (m, 2H), 1.73-1.59 (m, 2H), 1.35-1.31 (m, 9H), 1.08-1.05 (m, 1H), 0.73-0.71 (m, 2H), 0.02-0.03 (m, 1H).

Methyl (3S,6S,10aR,Z)-6-((tert-butoxycarbonyl)amino)-5-oxo-1,2,3,5,6,7,10,10a-octahydropyrrolo[1,2-a]azocine-3-carboxylate: LCMS (ESI) m/z=353.0 [M+H]⁺; ¹H NMR (400 MHz, DMSO-d₆) δ 6.75 (d, J=7.6 Hz, 0.9H), 6.22 (d, J=2.0 Hz, 0.1H), 4.33-4.25 (m, 2H), 4.14-4.12 (m, 1H), 3.60 (s, 3H), 2.20-2.10 (m, 1H), 2.08-2.03 (m, 3H), 1.85-1.84 (m, 1H), 1.74-1.57 (m, 2H), 1.38 (s, 9H), 1.34-1.14 (m, 3H), 0.75-0.73 (m, 1H), 0.03-0.01 (m, 1H).

### Step 4: Preparation of 3S,6S,7aS,8aR,9aR)-6-((tert-butoxycarbonyl)amino)-5-oxodecahydro-1H-cyclopropa[d]pyrrolo[1,2-a]azocine-3-carboxylic acid

To a solution of methyl (3S,6S,7aS,8aR,9aR)-6-((tert-butoxycarbonyl)amino)-5-oxodecahydro-1H-cyclopropa[d]pyrrolo[1,2-a]azocine-3-carboxylate (2.00 g, 5.67 mmol, 1 eq) in a mixture of MeOH (1.0 mL), THF (1.0 mL), and water (1.0 mL) was added LiOH·H₂O (270 mg, 11.3 mmol, 2 eq). After stirring for 2 h at ambient temperatures, the reaction mixture turned murky white. The reaction mixture was carefully concentrated under reduced pressure to give a residue. The residue was diluted with water (50 mL) and extracted with EtOAc (2×50 mL). The aqueous phase was acidified between pH˜4 to 5 with aqueous 1N HCl. The resulting acidic phase was extracted with EtOAc (2×50 mL) and the combined organic layers were concentrated under reduced pressure to give a residue. The product (3S,6S,7aS,8aR,9aR)-6-((tert-butoxycarbonyl)amino)-5-oxodecahydro-1H-cyclopropa[d]pyrrolo[1,2-a]azocine-3-carboxylic acid (2.00 g) was isolated as a yellow oil and used without further purification or manipulation. LCMS (ESI) m/z=339.0 [M+H]⁺.

The intermediate shown in Table 1 were synthesized using the appropriate starting materials and reagents under conditions described above for the preparation of (3S,6S,7aS,8aR,9aR)-6-((tert-butoxycarbonyl)amino)-5-oxodecahydro-1H-cyclopropa[d]pyrrolo[1,2-a]azocine-3-carboxylic acid.

TABLE 1

| Name | Structure | LCMS |
|---|---|---|
| (3S,6S,7aR,8aS,9aR)-6-((tert- butoxycarbonyl)amino)-5- oxodecahydro-1H- cyclopropa[d]pyrrolo[1,2-a] azocine-3- carboxylic acid | 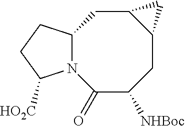 [SMILES: CC(C)(C)OC(=O)N[C@H]1C[C@H]2C[C@H]2C[C@H]2CC[C@@H](C(=O)O)N2C1=O] | 339.0 [M + H]⁺ |

Synthesis of methyl (3S,6S,9aR)-6-((tert-butoxycarbonyl)amino)-5-oxo-2,3,5,6,7,9a-hexahydro-1H-pyrrolo[1,2-a]azepine-3-carboxylate and methyl (3S,6S,9aR)-6-((tert-butoxycarbonyl)amino)-8-methyl-5-oxo-2,3,5,6,7,9a-hexahydro-1H-pyrrolo[1,2-a]azepine-3-carboxylate

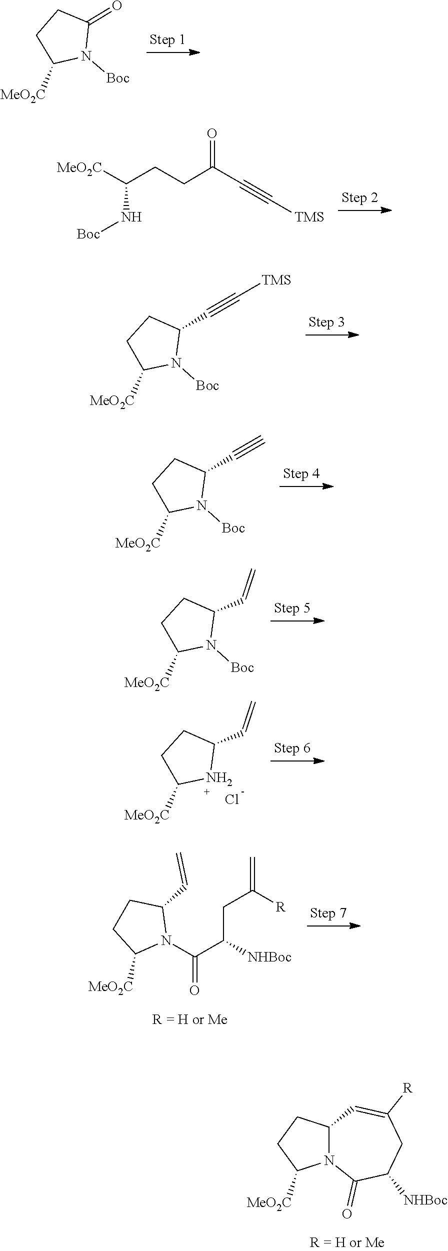

### Step 1: Preparation of methyl (S)-2-((tert-butoxycarbonyl)amino)-5-oxo-7-(trimethylsilyl)hept-6-ynoate

To a cooled (0° C.) solution of isopropylmagnesium chloride (2.0 M, 9.04 L, 1.10 eq) and THF (4.00 L) was added ethynyltrimethylsilane (1.86 kg, 18.9 mol, 2.62 L, 1.15 eq). The reaction mixture was stirred for 1 h, followed by slow addition of a solution of 1-(tert-butyl) 2-methyl (S)-5-oxopyrrolidine-1,2-dicarboxylate (4.00 kg, 16.4 mol, 1.00 eq) in THF (8.0 L) over 1.5 h. After stirring for an additional 30 min, the reaction mixture was transferred into a stirred and cooled (0° C.) biphasic mixture of iPAc (5.00 L) and 20% aqueous NH₄Cl (16.0 L). The biphasic mixture was separated, and the aqueous layer was extracted with iPAc (8.00 L). The combined organic layers were washed with 20% aqueous NH₄Cl solution (8.00 L), brine (8.00 L), dried over Na₂SO₄, filtered, and concentrated under reduced pressure to give methyl (S)-2-((tert-butoxycarbonyl)amino)-5-oxo-7-(trimethylsilyl)hept-6-ynoate (5.50 kg) as yellow oil. ¹H NMR (400 MHz, CDCl₃) δ 5.11-5.09 (m, 1H), 4.32-4.30 (m, 1H), 3.77-3.72 (m, 3H), 2.72-2.64 (m, 2H), 2.04-2.01 (m, 1H), 1.73-1.71 (m, 1H), 1.41 (s, 9H), 0.24 (s, 9H).

### Step 2: Preparation of 1-(tert-butyl) 2-methyl (2S,5R)-5-((trimethylsilyl)ethynyl)pyrrolidine-1,2-dicarboxylate

To a suspension of NaBH(OAc)₃ (4.03 kg, 19.0 mol, 1.30 eq) in iPAc (5.00 L) was added a solution of methyl (S)-2-((tert-butoxycarbonyl)amino)-5-oxo-7-(trimethylsilyl)hept-6-ynoate in iPAc (20.0 L). The batch was cooled to (−10° C.), followed by slow addition of TFA (7.18 kg, 62.9 mol, 4.66 L, 4.30 eq) over 1.5 h. The mixture was warmed to 10° C. and stirred for an additional 2 h. The reaction mixture was transferred to a cooled (0° C.) solution of 25% aqueous K₂HPO₄ (40.0 L). The pH of the suspension was adjusted to pH 6-7 with aqueous saturated NaHCO₃. The biphasic mixture was separated, and the aqueous layer was extracted with iPAc (8.00 L). The combined organic layers were washed with 25% K₂HPO₄ solution (8.00 L), brine (5.00 L), dried over Na₂SO₄, filtered, and concentrated under reduced pressure to give a residue. The residue was purified by silica gel column chromatography (petroleum ether/EtOAc=30/1 to 3/1) to yield 1-(tert-butyl) 2-methyl (2S,5R)-5-((trimethylsilyl)ethynyl)pyrrolidine-1,2-dicarboxylate (2.27 kg, 6.97 mol, 42.4% yield) as yellow oil. ¹H NMR (400 MHz, DMSO-d₆) δ 4.49-4.32 (m, 1H), 4.16 (s, 1H), 3.63 (s, 3H), 2.16 (s, 2H), 1.95-1.88 (m, 2H), 1.40-1.32 (m, 9H), 0.13 (s, 9H).

### Step 3: Preparation of 1-(tert-butyl) 2-methyl (2S,5R)-5-ethynylpyrrolidine-1,2-dicarboxylate

To a cooled (0° C.) solution of 1-(tert-butyl) 2-methyl (2S,5R)-5-((trimethylsilyl)ethynyl)pyrrolidine-1,2-dicarboxylate (500 g, 1.54 mol, 1.00 eq) in THF (2.00 L) was added a 1.0 M solution of TBAF (1.84 L, 1.20 eq) in THF and the mixture was subsequently stirred for 1 h. Four batches of equal scale were performed in parallel, and the reaction mixtures were combined and concentrated under reduced pressure to give a residue. The residue was purified by silica gel column chromatography (petroleum ether/EtOAc=1/0 to 0/1) to yield 1-(tert-butyl) 2-methyl (2S,5R)-5-ethynylpyrrolidine-1,2-dicarboxylate (920 g, 2.95 mol, 48.0% yield, 81.2% purity) as yellow oil. ¹H NMR (400 MHz, CDCl₃) δ 4.62-4.50 (m, 1H), 4.30-4.20 (m, 1H), 3.74-3.70 (m, 3H), 2.31-2.14 (m, 5H), 1.47-1.40 (m, 9H).

### Step 4: Preparation of 1-(tert-butyl) 2-methyl (2S,5R)-5-vinylpyrrolidine-1,2-dicarboxylate

To a suspension of 1-(tert-butyl) 2-methyl (2S,5R)-5-ethynylpyrrolidine-1,2-dicarboxylate (150 g, 592 mmol, 1.00 eq) in EtOAc (1.50 L) under N₂ (g) was added Lindlar's catalyst (7.50 g, 1.82 mmol, 5.0% wt) and quinoline (163 g, 1.27 mol, 150 mL, 2.14 eq). The suspension was degassed under vacuum and purged with H₂ (g) (3×). The mixture was stirred under H₂ (g) (50 psi) for 1 h. Six batches of equal scale were performed in parallel, filtered, and the filtrates were combined during workup. The mixture was filtered, the filtrate was washed with aqueous 1N HCl (9.00 L), separated, and concentrated. The combined residues were purified by silica gel column chromatography (petroleum ether/EtOAc=1/0 to 0/1) to yield 1-(tert-butyl) 2-methyl (2S,5R)-5-vinylpyrrolidine-1,2-dicarboxylate (745 g, 2.23 mol, 62.8% yield, 76.5% purity) as yellow oil. ¹H NMR (400 MHz, DMSO-d₆) δ 5.84-5.76 (m, 1H), 5.34-5.27 (m, 1H), 5.06-5.04 (m, 1H), 4.29-4.17 (m, 2H), 3.65-3.62 (m, 3H), 2.16-2.14 (m, 2H), 1.79-1.78 (m, 1H), 1.69-1.65 (m, 1H), 1.34-1.32 (m, 9H).

### Step 5: Preparation of methyl (2S,5R)-5-vinylpyrrolidine-2-carboxylate

To a solution of 1-(tert-butyl) 2-methyl (2S,5R)-5-vinylpyrrolidine-1,2-dicarboxylate (245 g, 959 mmol, 1.00 eq) in EtOAc (1.25 L) was added a solution of 4.0 M solution of HCl in EtOAc (959 mL, 4.00 eq). Three batches of equal scale were performed in parallel, and the mixture was stirred at 25° C. for 2 h. The reaction mixtures were combined and concentrated under reduced pressure to give product methyl (2S,5R)-5-vinylpyrrolidine-2-carboxylate (520 g, HCl) was obtained as an off-white solid. ¹H NMR (400 MHz, DMSO-d₆) δ 6.03-5.95 (m, 1H), 5.44-5.33 (m, 2H), 4.49-4.45 (m, 1H), 4.11-4.05 (m, 1H), 3.76 (s, 3H), 2.31-2.28 (m, 1H), 2.16-2.10 (m, 1H), 1.81-1.76 (m, 1H).

### Step 6: Preparation of methyl (2S,5R)-1-((S)-2-((tert-butoxycarbonyl)amino)pent-4-enoyl)-5-vinylpyrrolidine-2-carboxylate

To a solution of (S)-2-((tert-butoxycarbonyl)amino)pent-4-enoic acid (35.9 g, 167 mmol, 1.00 eq) in CH₂Cl₂ (320 mL) was added methyl (2S,5R)-5-vinylpyrrolidine-2-carboxylate (32.0 g, 167 mmol, 1.00 eq, HCl) and Et₃N (69.7 mL, 0.501 mol, 3.00 eq). The mixture was cooled to 0° C. and CMPI (98.8 g, 387 mmol, 1.10 eq) was added. The reaction mixture was subsequently warmed to 25° C. and stirred for 3 h. The reaction mixture was poured into water (500 mL), extracted with CH₂Cl₂ (200 mL×3). The combined organic layers were washed with saturated aqueous NH₄Cl (300 mL×2), dried over Na₂SO₄, filtered, and concentrated under reduced pressure to give a residue. The residue was purified by column chromatography (petroleum ether/EtOAc=50/1 to 5/1) to give methyl (2S,5R)-1-((S)-2-((tert-butoxycarbonyl)amino)pent-4-enoyl)-5-vinylpyrrolidine-2-carboxylate (47.0 g) as yellow oil. LCMS (ESI) m/z=367.2 [M+H]⁺; ¹H NMR (400 MHz, CDCl₃) δ 6.03-5.94 (m, 1H), 5.77-5.66 (m, 1H), 5.52 (d, J=16.8 Hz, 1H), 5.22 (d, J=10.4 Hz, 1H), 5.12-5.00 (m, 4H), 4.81 (br d, J=6.4 Hz, 1H), 4.53-4.44 (m, 2H), 3.73 (s, 3H), 2.45-2.38 (m, 1H), 2.32-2.27 (m, 1H), 2.22-2.15 (m, 2H), 2.02-1.83 (m, 3H), 1.41 (s, 9H).

The intermediates shown in Table 2 were synthesized using methyl (2S,5R)-5-vinylpyrrolidine-2-carboxylate, appropriate N-protected amino acids, CMPI, triethylamine, and CH₂Cl₂ under the reaction conditions described above. The intermediate(s) was purified using standard methods.

TABLE 2

| Name | Structure | LCMS |
|---|---|---|
| (2S,5R)-1-((S)-2- ((tert-butoxycarbonyl) amino)- 4-methylpent-4-enoyl)-5- vinylpyrrolidine-2- carboxylic acid | 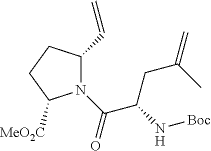 [SMILES: C=C[C@H]1CC[C@@H](C(=O)OC)N1C(=O)[C@H](CC(=C)C)NC(=O)OC(C)(C)C] | 367.2 [M + H]⁺ |

### Step 7: Preparation of methyl (3S,6S,9aR)-6-((tert-butoxycarbonyl)amino)-5-oxo-2,3,5,6,7,9a-hexahydro-1H-pyrrolo[1,2-a]azepine-3-carboxylate

To a solution of methyl (2S,5R)-1-((S)-2-((tert-butoxycarbonyl)amino)pent-4-enoyl)-5-vinylpyrrolidine-2-carboxylate (21.0 g, 59.6 mmol, 1.00 eq) in CH₂Cl₂ (2.10 L) was added 1st generation Grubb's catalyst (4.90 g, 5.96 mmol, 0.10 eq). The mixture was heated to 50° C. and stirred for 12 h. Two batches of equal scale were performed in parallel. The reaction mixtures were subsequently combined and concentrated to give a residue. The residue was purified by column chromatography (petroleum ether/EtOAc=10/1 to 1/1) to give the methyl (3S,6S,9aR)-6-((tert-butoxycarbonyl)amino)-5-oxo-2,3,5,6,7,9a-hexahydro-1H-pyrrolo[1,2-a]azepine-3-carboxylate (35.0 g, 94.8 mmol, 56.6% yield, 87.9% purity) as a gray solid. LCMS (ESI) m/z=225.0 [(M-Boc)+H]⁺; ¹H NMR (400 MHz, CDCl₃) δ 5.78-5.71 (m, 2H), 5.55 (d, J=11.6 Hz, 1H), 4.75-4.63 (m, 3H), 3.72 (s, 3H), 2.64-2.58 (m, 1H), 2.38-2.29 (m, 2H), 2.11-2.05 (m, 2H), 1.93-1.85 (m, 1H), 1.44 (s, 9H).

The intermediates shown in Table 3 were synthesized using the appropriate starting materials and reagents under conditions described above for the preparation of methyl (3S,6S,9aR)-6-((tert-butoxycarbonyl)amino)-5-oxo-2,3,5,6,7,9a-hexahydro-1H-pyrrolo[1,2-a]azepine-3-carboxylate.

TABLE 3

| Name | Structure | LCMS |
|---|---|---|
| methyl (3S,6S,9aR)-6- ((tert- butoxycarbonyl)amino)- 8-methyl-5-oxo- 2,3,5,6,7,9a-hexahydro- 1H-pyrrolo[1,2- a]azepine-3-carboxylate | 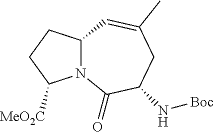 [SMILES: COC(=O)[C@@H]1CC[C@@H]2C=C(C)C[C@H](NC(=O)OC(C)(C)C)C(=O)N21] | 361.1 [M + Na]⁺ |

Synthesis of (3S,6S,7aS,8aS,8bR)-6-((tert-butoxycarbonyl)amino)-7a-methyl-5-oxodecahydrocyclopropa[c]pyrrolo[1,2-a]azepine-3-carboxylic acid and (3S,6S,7aR,8aR,8bR)-6-((tert-butoxycarbonyl)amino)-7a-methyl-5-oxodecahydrocyclopropa[c]pyrrolo[1,2-a]azepine-3-carboxylic acid

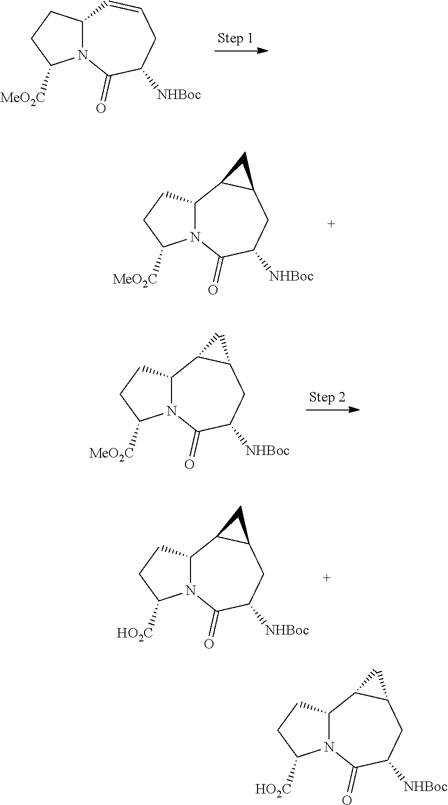

Step 1: Preparation of methyl (3S,6S,7aS,8aS,8bR)-6-((tert-butoxycarbonyl)amino)-5-oxodecahydrocyclopropa[c]pyrrolo[1,2-a]azepine-3-carboxylate and methyl (3S,6S,7aR,8aR,8bR)-6-((tert-butoxycarbonyl)amino)-5-oxodecahydrocyclopropa[c]pyrrolo[1,2-a]azepine-3-carboxylate

To a stirred and cooled (−40° C.) solution of 0.5 M diazomethane (1.29 g, 30.8 mmol, 10 eq) in MTBE (40 mL) was added a solution of methyl (3S,6S,9aR)-6-((tert-butoxycarbonyl)amino)-5-oxo-2,3,5,6,7,9a-hexahydro-1H-pyrrolo[1,2-a]azepine-3-carboxylate (1.00 g, 3.08 mmol, 1 eq) in DCM. To the cooled mixture was added Pd(OAc)₂ (6.91 mg, 30.8 μmol, 0.01 eq) in DCM. The mixture was subsequently allowed to warm up to −10° C. and effervescence of N₂ (g) gas observed. The reaction mixture was allowed to age at this temperature 2 h or until evolution of N₂ (g) ceased. The reaction mixture as warmed to room temperature and filtered. The filtrate was concentrated to give crude product as a mixture of diastereomers. The resulting mixture was purified by HPLC and mixture was separated to afford methyl (3S,6S,7aS,8aS,8bR)-6-((tert-butoxycarbonyl)amino)-5-oxodecahydrocyclopropa[c]pyrrolo[1,2-a]azepine-3-carboxylate (100 mg, 295 μmol, 9.61%) and methyl (3S,6S,7aR,8aR,8bR)-6-((tert-butoxycarbonyl)amino)-5-oxodecahydrocyclopropa[c]pyrrolo[1,2-a]azepine-3-carboxylate (110 mg, 325 μmol, 10.5

methyl (3S,6S,7aS,8aS,8bR)-6-((tert-butoxycarbonyl)amino)-5-oxodecahydrocyclopropa[c]pyrrolo[1,2-a]azepine-3-carboxylate: LCMS (ESI) m/z=239.0 [(M-Boc)+H]⁺.

methyl (3S,6S,7aR,8aR,8bR)-6-((tert-butoxycarbonyl)amino)-5-oxodecahydrocyclopropa[c]pyrrolo[1,2-a]azepine-3-carboxylate: LCMS (ESI) m/z=239.0 [(M-Boc)+H]⁺.

Step 2: Preparation of (3S,6S,7aS,8aS,8bR)-6-((tert-butoxycarbonyl)amino)-5-oxodecahydrocyclopropa[c]pyrrolo[1,2-a]azepine-3-carboxylic acid and (3S,6S,7aR,8aR,8bR)-6-((tert-butoxycarbonyl)amino)-5-oxodecahydrocyclopropa[c]pyrrolo[1,2-a]azepine-3-carboxylic acid

The intermediates shown in Table 4 were synthesized using the appropriate starting materials and LiOH·H₂O under the hydrolysis conditions described in Step 4 for the synthesis of (3S,6S,7aS,8aR,9aR)-6-((tert-butoxycarbonyl)amino)-5-oxodecahydro-1H-cyclopropa[d]pyrrolo[1,2-a]azocine-3-carboxylic acid and/or (3S,6S,7aR,8aS,9aR)-6-((tert-butoxycarbonyl)amino)-5-oxodecahydro-1H-cyclopropa[d]pyrrolo[1,2-a]azocine-3-carboxylic acid.

TABLE 4

| Name | Structure |
|---|---|
| (3S,6S,7aS,8aS,8bR)-6-((tert- butoxycarbonyl)amino)-7a-methyl-5- oxodecahydrocyclopropa[c]pyrrolo [1,2-a]azepine-3-carboxylic acid | 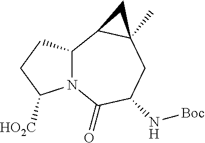 [SMILES: CC(C)(C)OC(=O)N[C@H]1C[C@]2(C)C[C@@H]2[C@H]2CC[C@@H](C(=O)O)N2C1=O] |
| (3S,6S,7aR,8aR,8bR)-6-((tert- butoxycarbonyl)amino)-7a-methyl-5- oxodecahydrocyclopropa[c]pyrrolo [1,2-a]azepine-3-carboxylic acid | 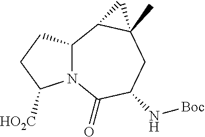 [SMILES: CC(C)(C)OC(=O)N[C@H]1C[C@@]2(C)C[C@H]2[C@H]2CC[C@@H](C(=O)O)N2C1=O] |

Synthesis of (3S,6S,7aS,8aR,9aR)-6-((tert-butoxycarbonyl)amino)-8,8-difluoro-5-oxodecahydro-1H-cyclopropa[d]pyrrolo[1,2-a]azocine-3-carboxylic acid

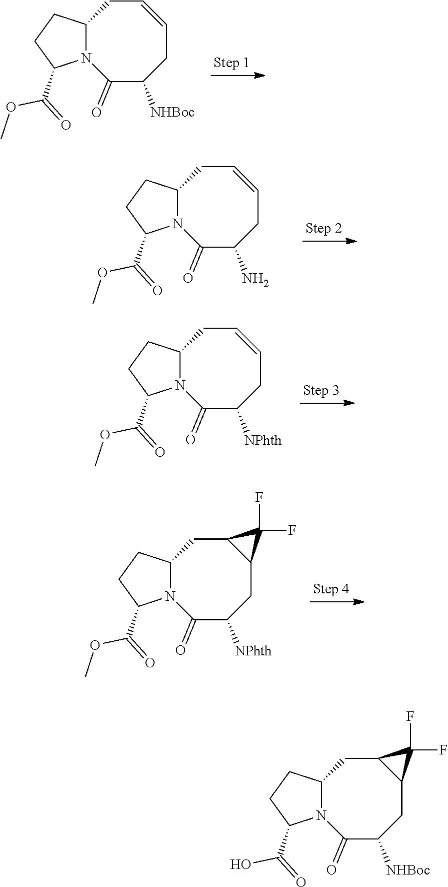

Step 1: Preparation of methyl (3S,6S,10aR,Z)-6-amino-5-oxo-1,2,3,5,6,7,10,10a-octahydropyrrolo[1,2-a]azocine-3-carboxylate

To a solution of methyl (3S,6S,10aR,Z)-6-((tert-butoxycarbonyl)amino)-5-oxo-1,2,3,5,6,7,10,10a-octahydropyrrolo[1,2-a]azocine-3-carboxylate (1.0 g, 2.95 mmol, 1 eq) in dichloromethane (15 mL) was added trifluoroacetic acid (4.0 mL, 52 mmol, 17 eq). The reaction mixture was stirred at room temperature 1 h. The reaction mixture was concentrated to dryness and purified directly by reverse phase chromatography using a gradient of 5-100% MeCN in basic water (10 mM NH₄HCO₃, pH=10) to afford methyl (3S,6S,10aR,Z)-6-amino-5-oxo-1,2,3,5,6,7,10,10a-octahydropyrrolo[1,2-a]azocine-3-carboxylate (588 mg, 2.46 mmol, 83% yield) as a white solid. ¹H NMR (400 MHz, CD₃OD) δ 5.78-5.64 (m, 2H), 4.45-4.41 (m, 1H), 4.25-4.17 (m, 1H), 4.11 (dd, 1H, J=9.7, 6.0 Hz), 3.66 (s, 3H), 2.81-2.67 (m, 2H), 2.41-2.33 (m, 1H), 2.22-2.05 (m, 3H), 1.97-1.86 (m, 2H).

Step 2: Preparation of methyl (3S,6S,10aR,Z)-6-(1,3-dioxoisoindolin-2-yl)-5-oxo-1,2,3,5,6,7,10,10a-octahydropyrrolo[1,2-a]azocine-3-carboxylate

A solution of methyl (3S,6S,10aR,Z)-6-amino-5-oxo-1,2,3,5,6,7,10,10a-octahydropyrrolo[1,2-a]azocine-3-carboxylate (500 mg, 2.09 mmol, 1 eq) and phthalic anhydride (318 mg, 2.15 mmol, 1.03 eq) in toluene (20 mL) was heated at reflux for 24 h. The solvent was then evaporated and the product was directly purified by reverse phase chromatography using a gradient of 5-100% MeCN in water to afford methyl (3S,6S,10aR,Z)-6-(1,3-dioxoisoindolin-2-yl)-5-oxo-1,2,3,5,6,7,10,10a-octahydropyrrolo[1,2-a]azocine-3-carboxylate (659 mg, 1.78 mmol, 85% yield) as an off-white solid. ¹H NMR (400 MHz, CDCl₃) δ 7.86-7.80 (m, 2H), 7.72-7.66 (m, 2H), 6.18-6.09 (m, 1H), 5.88-5.81 (m, 1H), 5.31 (dd, J=7.9, 4.5 Hz, 1H), 4.46-4.38 (m, 2H), 3.70 (s, 3H), 3.13-3.03 (m, 1H), 2.96-2.88 (m, 1H), 2.63-2.54 (m, 1H), 2.49-2.40 (m, 1H), 2.32-2.23 (m, 1H), 2.22-2.12 (m, 1H), 2.05-1.94 (m, 1H), 1.92-1.84 (m, 1H).

Step 3: Preparation of methyl (3S,6S,7aS,8aR,9aR)-6-(1,3-dioxoisoindolin-2-yl)-8,8-difluoro-5-oxodecahydro-1H-cyclopropa[d]pyrrolo[1,2-a]azocine-3-carboxylate

A round-bottomed flask equipped with a magnetic stirrer and a water condenser was charged with methyl (3S,6S,10aR,Z)-6-(1,3-dioxoisoindolin-2-yl)-5-oxo-1,2,3,5,6,7,10,10a-octahydropyrrolo[1,2-a]azocine-3-carboxylate (150 mg, 0.407 mmol, 1 eq), sodium fluoride (5.12 mg, 0.122 mmol, 0.3 eq) and anhydrous toluene (2 mL). The mixture was heated to reflux for 30 min. and then a solution of trimethylsilyl 2,2-difluoro-2-(fluorosulfonyl)acetate (159 μL, 0.814 mmol, 2 eq) in toluene (2 mL) was added via syringe pump over 1 h. The clear solution turned pale orange. The reaction was left stirring at reflux for 1.5 h then more trimethylsilyl 2,2-difluoro-2-(fluorosulfonyl)acetate (159 μL, 0.814 mmol, 2 eq) in toluene (2 mL) was added via syringe pump over 40 min. and the reaction was left stirring at reflux for 1 h. The reaction mixture was cooled to room temperature, diluted with aqueous saturated sodium bicarbonate solution and EtOAc and stirred for 10 min. The layers were separated. The organic layer was washed with brine, dried over sodium sulfate, filtered and concentrated to dryness to afford a crude orange foam (238 mg, 140%). A purification by reverse phase chromatography eluting with 5-100% MeCN in water (with 0.1% formic acid) afforded methyl (3S,6S,7aS,8aR,9aR)-6-(1,3-dioxoisoindolin-2-yl)-8,8-difluoro-5-oxodecahydro-1H-cyclopropa[d]pyrrolo[1,2-a]azocine-3-carboxylate (48 mg, 0.114 mmol, 28% yield). ¹H NMR (400 MHz, CDCl₃) δ 7.86-7.81 (m, 2H), 7.74-7.68 (m, 2H), 5.25-5.20 (m, 1H), 4.40 (t, J=8.1 Hz, 1H), 4.28-4.20 (m, 1H), 3.72 (s, 3H), 2.74-2.61 (m, 2H), 2.38-1.80 (m, 8H).

Step 4: Preparation of (3S,6S,7aS,8aR,9aR)-6-((tert-butoxycarbonyl)amino)-8,8-difluoro-5-oxodecahydro-1H-cyclopropa[d]pyrrolo[1,2-a]azocine-3-carboxylic acid

To a solution of methyl (3S,6S,7aS,8aR,9aR)-6-(1,3-dioxoisoindolin-2-yl)-8,8-difluoro-5-oxodecahydro-1H-cyclopropa[d]pyrrolo[1,2-a]azocine-3-carboxylate (145 mg, 0.346 mmol, 1 eq) in water (2 mL) and tetrahydrofuran (6 mL) was added lithium hydroxide monohydrate (43.2 mg, 1.03 mmol, 3 eq). The reaction was stirred at 20° C. for 1 h. The volatiles were removed in vacuo. The aqueous residue was diluted with 3 M aqueous hydrochloric acid (5 mL) and the solution was heated to 100° C. for 20 h. The reaction mixture was concentrated to dryness. The residue was diluted with 1 M aqueous NaOH (6 mL) and di-tert-butyl dicarbonate (377 mg, 1.73 mmol, 5 eq) was added. The reaction was stirred at 20° C. for 20 h. More di-tert-butyl dicarbonate (800 mg) was added and the reaction was stirred at 20° C. for 16 h. The volatiles were removed in vacuo. The resulting aqueous solution was acidified to pH 2 using a 6 N aqueous solution of HCl and the product was extracted with EtOAc (2×15 mL). The combined organic layers were washed with brine, dried over sodium sulfate, filtered and concentrated under reduced pressure. The product was purified by reverse phase chromatography with a gradient of 5-100% MeCN in water (with 0.1% formic acid) to afford (3S,6S,7aS,8aR,9aR)-6-((tert-butoxycarbonyl)amino)-8,8-difluoro-5-oxodecahydro-1H-cyclopropa[d]pyrrolo[1,2-a]azocine-3-carboxylic acid (84.0 mg, 0.224 mmol, 65%) as a white solid. ¹H NMR (400 MHz, CDCl₃) δ 5.40-5.33 (m, 1H), 4.72 (d, J=7.4 Hz, 1H), 4.64 (q, J=8.5 Hz, 1H), 4.28-4.18 (m, 1H), 2.43-2.26 (m, 2H), 2.25-2.16 (m, 1H), 2.11-1.90 (m, 5H), 1.74-1.63 (m, 2H), 1.43 (s, 9H).

### Synthesis of Linkers

The following intermediates in Table 5 were prepared according to the protocol described in WO 2020205467.

TABLE 5

| Name | Structure |
|---|---|
| 5-((diethoxyphosphoryl) difluoromethyl)benzo[b] thiophene-2-carboxylic acid | 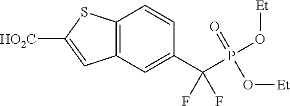 [SMILES: CCOP(=O)(OCC)C(F)(F)c1ccc2sc(C(=O)O)cc2c1] |
| 7-((diethoxyphosphoryl) difluoromethyl)-2- naphthoic acid | 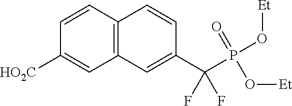 [SMILES: CCOP(=O)(OCC)C(F)(F)c1ccc2ccc(C(=O)O)cc2c1] |

### Representative Procedure for Preparation of Activated Ester Phosphonic Acids

Synthesis of (difluoro(2-((4-nitrophenoxy)carbonyl)benzo[b]thiophen-5-yl)methyl)phosphonic acid

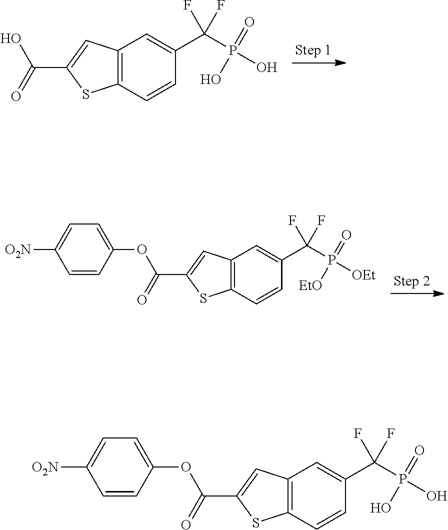 [SMILES: O=C(O)c1cc2cc(C(F)(F)P(=O)(O)O)ccc2s1 ; CCOP(=O)(OCC)C(F)(F)c1ccc2sc(C(=O)Oc3ccc([N+](=O)[O-])cc3)cc2c1 ; O=C(Oc1ccc([N+](=O)[O-])cc1)c1cc2cc(C(F)(F)P(=O)(O)O)ccc2s1]

Step 1: Preparation of 4-nitrophenyl 5-((diethoxyphosphoryl)difluoromethyl)benzo[b]thiophene-2-carboxylate

To a mixture of 5-[(diethoxyphosphoryl)difluoromethyl]-1-benzothiophene-2-carboxylic acid (L1) (10.0 g, 27.4 mmol), EDCI (7.85 g, 41.0 mmol) and DMAP (836 mg, 6.85 mmol) in CH₂Cl₂ (80 mL) was stirred at room temperature. After 15 min, 4-nitrophenol (4.75 g, 34.2 mmol) was added and the resulting yellow mixture was stirred at room temperature for 18 h. The reaction was quenched with water (30 mL) and the product was extracted with CH₂Cl₂ (10 mL×2). The combined organic extracts were washed with brine, dried with sodium sulfate, filtered, and concentrated in vacuo. The crude residue was purified by reverse phase chromatography [C18 cartridge eluting with a gradient of 5-100% acetonitrile in water] and the appropriate fractions were concentrated to give 4-nitrophenyl 5-((diethoxyphosphoryl)difluoromethyl)benzo[b]thiophene-2-carboxylate (7.80 g, 16.0 mmol, 59.0% yield) as a yellow solid. LCMS m/z=486.2 [M+H]⁺; ¹H NMR (400 MHz, DMSO-d₆) δ 8.32-8.37 (m, 3H), 8.22 (s, 1H), 8.01 (d, J=9.1 Hz, 1H), 7.77 (d, J=7.8 Hz, 1H), 7.51-7.45 (m, 2H), 4.14-4.32 (m, 4H), 1.34 (t, J=7.8 Hz, 6H).

Step 2: Preparation of (difluoro(2-((4-nitrophenoxy)carbonyl)benzo[b]thiophen-5-yl)methyl)phosphonic acid

To a cooled (0° C.) solution of 4-nitrophenyl 5-((diethoxyphosphoryl)difluoromethyl)benzo[b]thiophene-2-carboxylate (4.47 g, 9.20 mmol) in CH₂Cl₂ (39 mL) was added N,O-bis(trimethylsilyl)trifluoroacetamide (12.1 mL, 46.0 mmol) and iodotrimethylsilane (5.23 mL, 36.8 mmol) as a solution in CH₂Cl₂ (10 mL). The reaction mixture was gradually allowed to warm to ambient temperatures. To the reaction mixture was added a mixture of 2:1 H₂O/acetonitrile (with 0.1% TFA) (50 mL) and precipitation of product was observed. The volatiles were removed in vacuo and the crude residue was suspended in a mixture of acetonitrile/water solution (1:1 v/v, 100 mL). The suspension was filtered, the solids were washed with a 2:1 mixture acetonitrile/water solution, and the solid were dried under reduced pressure to afford (difluoro(2-((4-nitrophenoxy)carbonyl)benzo[b]thiophen-5-yl)methyl)phosphonic acid (6.5 g, 94%) as a beige solid. The filtrate was concentrated to 50% of solvent volume and the resulting suspension was filtered and washed with 1:2 acetonitrile/water solution. The solid was dried under reduced pressure to afford additional (difluoro(2-((4-nitrophenoxy)carbonyl)benzo[b]thiophen-5-yl)methyl)phosphonic acid (0.4 g) as a beige solid. Both products were lyophilized to give (difluoro(2-((4-nitrophenoxy)carbonyl)benzo[b]thiophen-5-yl)methyl)phosphonic acid (6.90 g, 16.0 mmol, 98.0% yield). ¹H NMR (400 MHz, DMSO-d₆) δ 7.66-7.74 (m, 3H), 8.27 (d, J=8.3 Hz, 1H), 8.30 (s, 1H), 8.36-8.41 (m, 2H), 8.66 (s, 1H).

The following intermediates in Table 6 were prepared using a similar protocol outlined above for synthesis of (difluoro(2-((4-nitrophenoxy)carbonyl)benzo[b]thiophen-5-yl)methyl)phosphonic acid and utilizing the appropriate advanced intermediate(s) as starting material(s).

TABLE 6

| Name | Structure | LC-MS |
|---|---|---|
| (difluoro(7-((4- nitrophenoxy)carbonyl)naphthalen-2- yl)methyl)phosphonic acid | 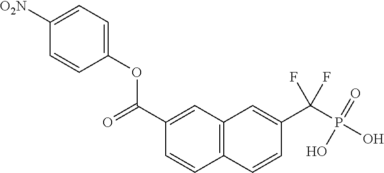 [SMILES: O=C(Oc1ccc([N+](=O)[O-])cc1)c1ccc2ccc(C(F)(F)P(=O)(O)O)cc2c1] | 424.1 [M + H]⁺ |
| (difluoro(2- ((perfluorophenoxy)carbonyl)benzo[b] thiophen-5-yl)methyl)phosphonic acid | 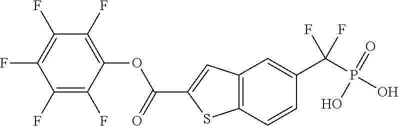 [SMILES: O=C(Oc1c(F)c(F)c(F)c(F)c1F)c1cc2cc(C(F)(F)P(=O)(O)O)ccc2s1] | 475.0 [M + H]⁺ |
| (difluoro(7- ((perfluorophenoxy)carbonyl) naphthalen- 2-yl)methyl)phosphonic acid | 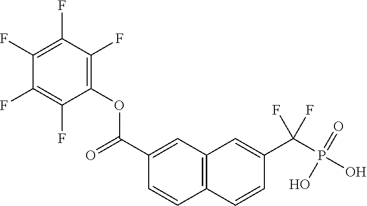 [SMILES: O=C(Oc1c(F)c(F)c(F)c(F)c1F)c1ccc2ccc(C(F)(F)P(=O)(O)O)cc2c1] | 467.0 [M − H]⁻ |

Synthesis of ((2-((perfluorophenoxy)carbonyl)benzo[b]thiophen-5-yl)methyl)phosphonic acid

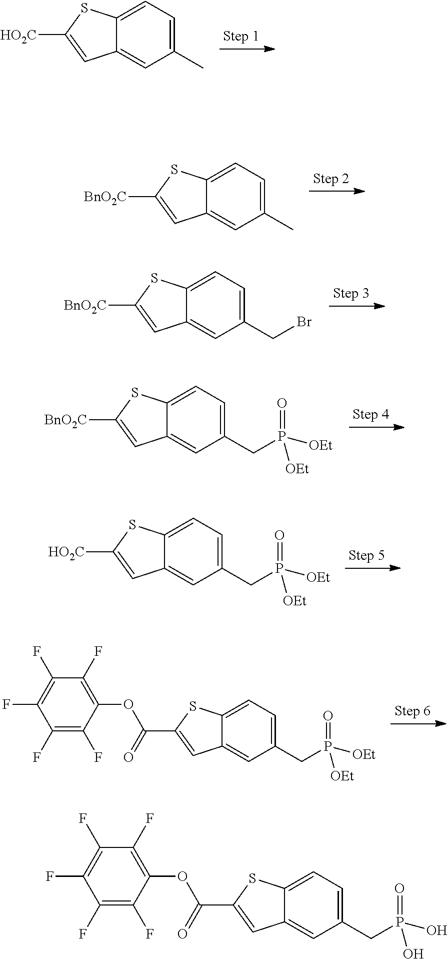

Preparation of 5-methylbenzo[b]thiophene-2-carboxylic acid

5-Methylbenzo[b]thiophene-2-carboxylic acid was prepared according to the procedure described in WO2016100184 A1.

Step 1: Preparation of benzyl 5-methylbenzo[b]thiophene-2-carboxylate

To a solution of 5-methylbenzo[b]thiophene-2-carboxylic acid (21.2 g, 110.0 mmol, 1.0 eq) and K₂CO₃ (30.4 g, 220.0 mmol, 2.0 eq) in DMF (200 mL) was added benzyl bromide (20.6 g, 121.0 mmol, 1.1 eq). The mixture was stirred at room temperature for 14 h. The reaction mixture was poured into ice water (400 mL) and stirred for 5 min. The resulting solids were filtered, and the filter cake was washed with water (50 mL), dried in vacuum to give benzyl 5-methylbenzo[b]thiophene-2-carboxylate (30.1 g, 106.0 mmol, 97% yield) as a yellow solid. ¹H NMR (400 MHz, CDCl₃) δ 8.01 (s, 1H), 7.71 (t, J=12.2 Hz, 1H), 7.65 (s, 1H), 7.46 (d, J=6.8 Hz, 2H), 7.42-7.35 (m, 3H), 7.29-7.26 (m, 1H), 5.38 (s, 2H), 2.47 (s, 3H).

Step 2: Preparation of benzyl 5-(bromomethyl)benzo[b]thiophene-2-carboxylate

To a solution of benzyl 5-methylbenzo[b]thiophene-2-carboxylate (15.0 g, 53.1 mmol, 1.0 eq) and NBS (10.3 g, 58.4 mmol, 1.1 eq) in CCl₄ (30 mL) was added benzoyl peroxide (1.3 g, 5.31 mmol, 0.1 eq). The reaction flask was subjected to three cycles of evacuation and backfilling with N₂ (g). The mixture was stirred at 80° C. for 16 h under constant atmosphere of N₂ (g). The reaction mixture was concentrated under reduced pressure. The residue was purified by flash column chromatography on silica gel to give benzyl 5-(bromomethyl)benzo[b]thiophene-2-carboxylate (6.80 g, 18.8 mmol, 36% yield) as a yellow solid.

Step 3: Preparation of benzyl 5-((diethoxyphosphoryl)methyl)benzo[b]thiophene-2-carboxylate

A solution of benzyl 5-(bromomethyl)benzo[b]thiophene-2-carboxylate (10.3 g, 28.5 mmol, 1.0 eq) dissolved in triethyl phosphite (30.0 g, 180.0 mmol, 6.3 eq) was stirred at 100° C. for 5 h. The reaction mixture was concentrated under reduced pressure directly, the residue was purified by flash column chromatography on silica gel to give benzyl 5-((diethoxyphosphoryl)methyl)benzo[b]thiophene-2-carboxylate (6.5 g, 15.5 mmol, 55% yield) as a colorless oil. LCMS (ESI) m/z=419 [M+H]⁺; ¹H NMR (400 MHz, CDCl₃) δ 8.04 (s, 1H), 7.80 (d, J=8.3 Hz, 2H), 7.49-7.33 (m, 6H), 5.39 (s, 2H), 4.08-3.93 (m, 4H), 3.26 (d, J=21.5 Hz, 2H), 1.24 (t, J=7.1 Hz, 6H).

Step 4: Preparation of 5-((diethoxyphosphoryl)methyl)benzo[b]thiophene-2-carboxylic acid

To a solution of benzyl 5-((diethoxyphosphoryl)methyl)benzo[b]thiophene-2-carboxylate (5.6 g, 13.3 mmol, 1.0 eq) in dissolved in a mixture of THF (80 mL) and H₂O (10 mL) was added LiOH (1.10 g, 26.6 mmol, 2.0 eq). The mixture was stirred at room temperature for 3 h and subsequently acidified with aqueous solution of 1 N HCl (adjusted to pH˜3-4). The product precipitated out of solution upon acidification. The resulting solids were filtered, the filter cake was washed with water (20 mL×2), and the solids were dried under vacuum to give 5-((diethoxyphosphoryl)methyl)benzo[b]thiophene-2-carboxylic acid (3.9 g, 11.8 mmol, 88.9% yield) as a white solid. LCMS (ESI) m/z=329 [M+H]⁺.

Step 5: Preparation of perfluorophenyl 5-((diethoxyphosphoryl)methyl)benzo[b]thiophene-2-carboxylate

To a cooled (0° C.) solution of 5-((diethoxyphosphoryl)methyl)benzo[b]thiophene-2-carboxylic acid (3.9 g, 11.8 mmol, 1.0 eq) in CH₂Cl₂ (50 mL) was added oxalyl chloride (2.2 g, 17.7 mmol, 1.5 eq) followed by addition of two drops of DMF. The mixture was stirred at 0° C. for 30 min, followed by evaporation of the reaction mixture to dryness. The resulting solids were dissolved in CH₂Cl₂ (50 mL), followed by addition of Et₃N (3.6 g, 35.4 mmol, 3.0 eq) and pentafluorophenol (2.6 g, 14.1 mmol, 1.2 eq). The resulting mixture was stirred at room temperature for additional 2 h and subsequently, poured over H₂O (30 mL). The bi-phasic solution was extracted with EtOAc (30 mL×3). The combined organic layers were dried over MgSO₄, filtered, and concentrated under reduced pressure, the residue was purified by column chromatography on silica gel to give perfluorophenyl 5-((diethoxyphosphoryl)methyl)benzo[b]thiophene-2-carboxylate (4.7 g, 9.5 mmol, 81% yield) as a white solid. LCMS (ESI) m/z=419 [M+H]⁺; ¹H NMR (400 MHz, CDCl₃) δ 8.29 (s, 1H), 7.88 (d, J=9.1 Hz, 2H), 7.51 (d, J=8.4 Hz, 1H), 4.14-3.94 (m, 4H), 3.29 (d, J=21.5 Hz, 2H), 1.26 (t, J=7.0 Hz, 6H).

Step 6: Preparation of ((2-((perfluorophenoxy)carbonyl)benzo[b]thiophen-5-yl)methyl)phosphonic acid

To a solution of perfluorophenyl 5-((diethoxyphosphoryl)methyl)benzo[b]thiophene-2-carboxylate (4.7 g, 9.5 mmol, 1.0 eq) in CH₂Cl₂ (60 mL) was added bromotrimethylsilane (12 mL). The mixture was stirred at room temperature for 14 h and subsequently concentrated under reduced pressure. The residue was purified by C18 column chromatography to give ((2-((perfluorophenoxy)carbonyl)benzo[b]thiophen-5-yl)methyl)phosphonic acid (3.7 g, 8.4 mmol, 89% yield) as a white solid. LCMS (ESI) m/z=439 [M+H]⁺.

Synthesis of (R)- or (S)-5-((diethoxyphosphoryl)fluoromethyl)benzo[b]thiophene-2-carboxylic acid and (S)-5-((diethoxyphosphoryl)fluoromethyl)benzo[b]thiophene-2-carboxylic acid

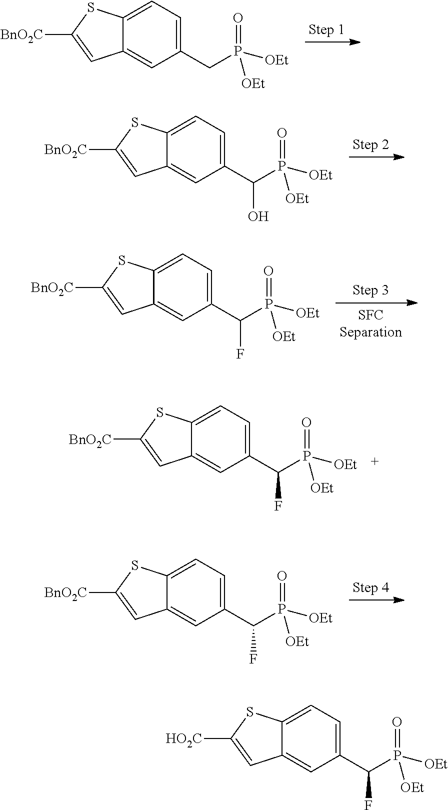

Step 1: rac-benzyl 5-((diethoxyphosphoryl)(hydroxy)methyl)benzo[b]thiophene-2-carboxylate

To a cooled (−78° C.) solution of benzyl 5-((diethoxyphosphoryl)methyl)benzo[b]thiophene-2-carboxylate (2.4 g, 5.73 mmol, 1 eq) in THF (75 mL) and 2-(benzenesulfonyl)-3-phenyloxaziridine (2.97 g, 11.4 mmol, 2 eq) was added a 1 M solution of NaHMDS (11.4 mL, 11.4 mmol, 2 eq) in THF. A deep purple solution was observed upon addition of base that changed to orange after complete addition of the base. The mixture was stirred for an additional 10 min, followed by addition of aqueous saturated NH₄Cl (50 mL). The mixture was warmed to ambient temperatures and EtOAc (75 mL) and water (25 mL) was added. After stirring for an additional 30 min, the phases were separated. The aqueous layer was extracted with EtOAc (125 mL×2). The combined organic extracts were dried with anhydrous sodium sulfate, filtered, concentrated under reduced pressure. Another batch of equal scale was performed and combined for purification. The combined material (6.42 mmol, 12.15 mmol in total) was purified by flash chromatography (20%-100%=EtOAc:heptane) to give rac-benzyl 5-((diethoxyphosphoryl)(hydroxy)methyl)benzo[b]thiophene-2-carboxylate (3.69 g, 8.49 mmol, 70%) as a white sticky solid. LCMS (ESI) m/z=869.4 [2M+H]⁺; ¹H NMR (400 MHz, CDCl₃) δ 8.10 (s, 1H), 8.04-8.00 (m, 1H), 7.88 (d, J=8.6 Hz, 1H), 7.61 (d, J=8.6 Hz, 1H), 7.51-7.47 (m, 2H), 7.46-7.35 (m, 3H), 5.42 (s, 2H), 5.17 (dd, J=10.4, 4.5 Hz, 1H), 4.18-3.95 (m, 4H), 3.10-2.99 (m, 1H), 1.33-1.20 (m, 6H).

Step 2: rac-benzyl 5-((diethoxyphosphoryl)fluoromethyl)benzo[b]thiophene-2-carboxylate

To a cooled (−78° C.) solution (under N₂ (g)) of rac-benzyl 5-((diethoxyphosphoryl)(hydroxy)methyl)benzo[b]thiophene-2-carboxylate (cc) (1.56 g, 3.59 mmol, 1 eq) in CH₂Cl₂ (30 mL) was added (diethylamino)sulfur trifluoride (568 μL, 4.30 mmol, 1.2 eq). The reaction was stirred for 15 min, followed by addition of aqueous saturated NaHCO₃ (50 mL). After warming to room temperature, the product was extracted with CH₂Cl₂ (50 mL×3). The combined organic layers were dried over sodium sulfate, filtered and concentrated under reduced pressure. The crude residue was purified by reverse phase chromatography on C18 cartridge (eluting with 5-80% acetonitrile in water) to give rac-benzyl 5-((diethoxyphosphoryl)fluoromethyl)benzo[b]thiophene-2-carboxylate (650 mg, 1.48 mmol, 41.6%) as a thick clear oil. LCMS (ESI) m/z=873.2 [2M+H]⁺; ¹H NMR (400 MHz, CDCl₃) δ 8.12 (s, 1H), 8.02-7.99 (m, 1H), 7.92 (d, J=8.7 Hz, 1H), 7.61 (d, J=8.7 Hz, 1H), 7.51-7.51 (m, 2H), 7.46-7.36 (m, 3H), 5.82 (dd, J=44.4, 7.5 Hz, 1H), 5.42 (s, 2H), 4.21-4.02 (m, 4H), 1.34-1.26 (m, 6H).

Step 3: Preparation of benzyl (R)-5-((diethoxyphosphoryl)fluoromethyl)benzo[b]thiophene-2-carboxylate and benzyl (S)-5-((diethoxyphosphoryl)fluoromethyl)benzo[b]thiophene-2-carboxylate

rac-Benzyl 5-((diethoxyphosphoryl)fluoromethyl)benzo[b]thiophene-2-carboxylate (650 mg, 1.48 mmol) was submitted to chiral SFC separation (Column: Lux i-Amylose 3, 21.2×250 mm 5 um column, 75 mL/min, 40% MeOH) to give benzyl (R)- or (S)-5-((diethoxyphosphoryl)fluoromethyl)benzo[b]thiophene-2-carboxylate (304 mg, 0.70 mmol, 46.8% recovery, 99.9% ee) as a thick clear oil (Peak 1) and benzyl (R)- or (S)-5-((diethoxyphosphoryl)fluoromethyl)benzo[b]thiophene-2-carboxylate (317 mg, 0.73 mmol, 49% recovery, 99.9% ee) as a thick clear oil (Peak 2).

Note: Fastest eluting enantiomer by SFC was arbitrarily assigned as (R)-5-(fluoro(phosphono)methyl)benzo[b]thiophene-2-carboxylic acid and slowest eluting enantiomer by SFC as (S)-5-(fluoro(phosphono)methyl)benzo[b]thiophene-2-carboxylic acid.

HPLC method for analysis of enantiomeric excess: Lux Cellulose-3 150 mm 45% H₂O+0.05% TFA/55% MeCN 1 mL/min 8 min.

Step 4: Preparation of (R)- or (S)-5-((diethoxyphosphoryl)fluoromethyl)benzo[b]thiophene-2-carboxylic acid

To a mixture of 10% Pd/C (60 mg, 50% wet) and benzyl (R)-5-((diethoxyphosphoryl)fluoromethyl)benzo[b]thiophene-2-carboxylate (Peak 1) (60 mg, 0.1374 mmol, 1 eq) in THF (5 mL) was degassed with N₂ (g) for 5 min. To the mixture was bubbled H₂ (g) for 5 min then the reaction was allowed to stir at room temperature under H₂ (g) (1 atm). The reaction mixture was stirred until consumption of starting material was detected by LCMS. The reaction mixture was subsequently sparged N₂ (g) for 15 min and filtered over a pad of Celite®. The filter cake was washed with 2-MeTHF and the filtrate was concentrated to give (R)- or (S)-5-((diethoxyphosphoryl)fluoromethyl)benzo[b]thiophene-2-carboxylic acid (47.4 mg, 0.0137 mmol, 99%) as a thick clear oil. LCMS (ESI) m/z=347.2 [M+H]⁺.

The following intermediate in Table 7 was prepared using the procedure outlined above (in Step 4) starting from benzyl (S)- or (R)-5-((diethoxyphosphoryl)fluoromethyl)benzo[b]thiophene-2-carboxylate (Peak 2) and using the appropriate reagents.

TABLE 7

| Name | Structure | LCMS |
|---|---|---|
| (S)-or (R)-5- ((diethoxy- phosphoryl) fluoromethyl) benzo[b] thiophene- 2-carboxylic acid | 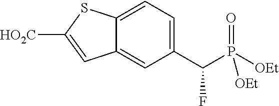 [SMILES: CCOP(=O)(OCC)[C@H](F)c1ccc2sc(C(=O)O)cc2c1] Or 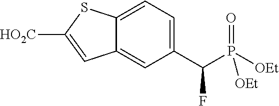 [SMILES: CCOP(=O)(OCC)[C@@H](F)c1ccc2sc(C(=O)O)cc2c1] | 347.2 [M + H]⁺ |

### Representative Methods for Synthesis of Activated Linkers

### Method 1: Stepwise Acid Chloride Method for Synthesis of Mixed Linkers

Representative Procedure for the synthesis of 4-nitrophenyl 5-(((2-(butyrylthio)ethoxy)(pyridin-3-yloxy)phosphoryl)difluoromethyl)benzo[b]thiophene-2-carboxylate

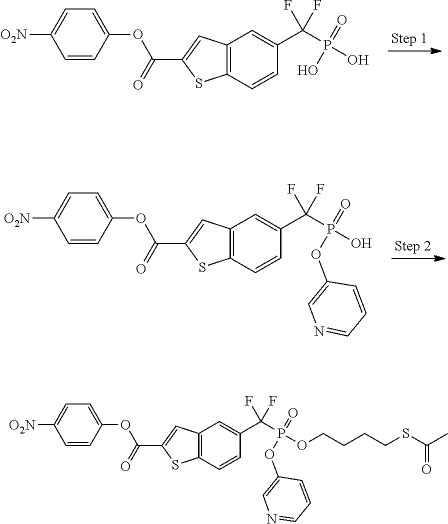 [SMILES: O=C(Oc1ccc([N+](=O)[O-])cc1)c1cc2cc(C(F)(F)P(=O)(O)O)ccc2s1 ; O=C(Oc1ccc([N+](=O)[O-])cc1)c1cc2cc(C(F)(F)P(=O)(O)Oc3cccnc3)ccc2s1 ; CC(=O)SCCCCOP(=O)(Oc1cccnc1)C(F)(F)c1ccc2sc(C(=O)Oc3ccc([N+](=O)[O-])cc3)cc2c1]

Step 1: Preparation of 4-nitrophenyl 5-(difluoro(hydroxy(pyridin-3-yloxy)phosphoryl)methyl)benzo[b]thiophene-2-carboxylate

To a cooled (0° C.) heterogeneous solution of (difluoro(2-((4-nitrophenoxy)carbonyl)benzo[b]thiophen-5-yl)methyl)phosphonic acid (100 mg, 0.2329 mmol, 1 eq) in CH₂Cl₂ (4 mL) was added catalytic DMF (2 drops) followed by dropwise addition of oxalyl chloride (198 μL, 2.32 mmol, 10 eq). The homogenous reaction mixture was warmed up to room temperature and stirred for 2 h. The reaction was concentrated in vacuo and further dried under high vacuum for 30 min to give a yellow solid. The yellow solid was diluted in CH₂Cl₂ (4 mL) and cooled down to −78° C. A solution of pyridin-3-ol (22.0 mg, 232 μmol, 1 eq) and triethylamine (64.7 μL, 465 μmol, 2 eq) in CH₂Cl₂ (1 mL) [sonicated for 1 min to allow for solubilization] and was added slowly. The homogeneous reaction mixture was stirred at −78° C. for 2 min, then allowed to warm to ambient temperatures and stirred overnight. After 24 h, the reaction mixture turned heterogeneous and the reaction was concentrated under reduced pressure. The crude product 4-nitrophenyl 5-(difluoro(hydroxy(pyridin-3-yloxy)phosphoryl)methyl)benzo[b]thiophene-2-carboxylate was used directly in the next step without further purification or manipulation. LCMS m/z=507.2 [M+H]⁺.

Step 2: Preparation of 4-nitrophenyl 5-(((2-(butyrylthio)ethoxy)(pyridin-3-yloxy)phosphoryl)difluoromethyl)benzo[b]thiophene-2-carboxylate

To a cooled (0° C.) solution of nitrophenyl 5-(difluoro(hydroxy(pyridin-3-yloxy)phosphoryl)methyl)benzo[b]thiophene-2-carboxylate (117 mg, 0.2329 mmol, 1 eq) in CH₂Cl₂ (5 mL) was added 2 drops of DMF followed by dropwise addition of oxalyl chloride (198 μL, 2.32 mmol, 10 eq). The reaction was warmed up to room temperature and stirred for 1.5 h. LCMS analysis showed partial conversion to desired activated intermediate. Additional oxalyl chloride (198 μL, 2.32 mmol, 10 eq) was introduced into the reaction mixture and the mixture was stirred for additional 1 h. The reaction was concentrated in vacuo and further dried under high vacuum for 30 min to give a yellow solid. The yellow solid was diluted in CH₂Cl₂ (5 mL) and cooled to −78° C. To the cooled solution was slowly added a solution of 1-[(2-hydroxyethyl)sulfanyl]butan-1-one (103 mg, 698 μmol, 3 eq) diluted in CH₂Cl₂ (1 mL) [previously dried by passing through anhydrous Na₂SO₄] followed by triethylamine (134 μL, 967 μmol, 2 eq). After stirring for 2 min, the resulting mixture was allowed to warm to ambient temperatures and stirred overnight. To the mixture was added Celite® and the mixture was carefully concentrated in vacuo. The crude residue was purified by flash-chromatography (gradient elution 0-60% EtOAc in heptane) to give 4-nitrophenyl 5-(((2-(butyrylthio)ethoxy)(pyridin-3-yloxy)phosphoryl)difluoromethyl)benzo[b]thiophene-2-carboxylate (22.0 mg, 0.03455 mmol, 14.9% yield) as a clear oil. LCMS m/z=637.2 [M+H]⁺; ¹H NMR: (400 MHz, DMSO-d₆) δ 8.50-8.44 (m, 2H), 8.33-8.38 (m, 3H), 8.25 (s, 1H), 8.04 (d, J=8.0 Hz, 1H), 7.79 (d, J=8.6 Hz, 1H), 7.57-7.53 (m, 1H), 7.50-7.45 (m, 2H), 7.31-7.26 (m, 1H), 4.37-4.24 (m, 2H), 3.20-3.08 (m, 2H), 2.52 (t, J=7.6 Hz, 2H), 1.67 (sextet, J=7.3 Hz, 2H), 0.94 (t, J=7.6 Hz, 3H).

The following intermediates in Table 8 were prepared using a similar protocol described above for synthesis of 4-nitrophenyl 5-(((2-(butyrylthio)ethoxy)(pyridin-3-yloxy)phosphoryl)difluoromethyl)benzo[b]thiophene-2-carboxylate and utilizing the appropriate advanced intermediate(s) as starting material(s).

TABLE 8

| Name | Structure | LCMS | NMR |
|---|---|---|---|
| 4-nitrophenyl 5- (((2- (butyrylthio)ethoxy) (phenoxy)phosphoryl) difluoromethyl)benzo[b] thiophene-2-carboxylate | 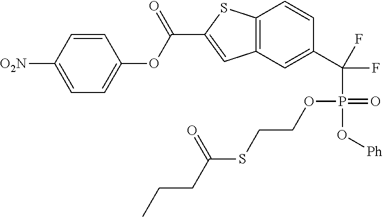 [SMILES: CCCC(=O)SCCOP(=O)(Oc1ccccc1)C(F)(F)c1ccc2sc(C(=O)Oc3ccc([N+](=O)[O-])cc3)cc2c1] | | ¹H NMR (400 MHz, DMSO-d₆) δ 8.67 (s, 1H), 8.44 (s, 1H), 8.40-8.35 (m, 3H), 7.79 (d, J = 8.8 Hz, 1H), 7.70 (d, J = 9.0 Hz, 2H), 7.44-7.37 (m, 2H), 7.29-7.23 (m, 1H), 7.18 (d, J = 8.6 Hz, 2H), 4.36- 4.20 (m, 2H), 3.15 (t, J = 6.1 Hz, 2H), 2.58-2.52 (m, 2H), 1.58-1.50 (m, 2H), 0.85 (q, J = 7.3 Hz, 3H) |
| 4-nitrophenyl 5- (((2- (butyrylthio) ethoxy) (naphthalen-1- yloxy)phosphoryl) difluoromethyl) benzo[b]thiophene- 2-carboxylate | 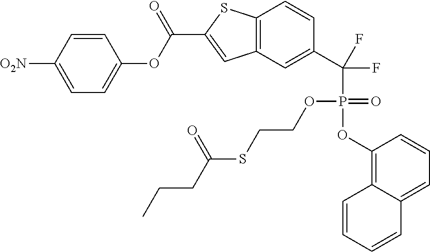 [SMILES: CCCC(=O)SCCOP(=O)(Oc1cccc2ccccc12)C(F)(F)c1ccc2sc(C(=O)Oc3ccc([N+](=O)[O-])cc3)cc2c1] | | ¹H NMR (400 MHz, DMSO-d₆) δ 8.65 (s, 1H), 8.49 (s, 1H), 8.39 (d, J = 9.3 Hz, 3H), 8.00 (d, J = 8.3 Hz, 1H), 7.87-7.80 (m, 3H), 7.70 (d, J = 9.0 Hz, 2H), 7.64-7.58 (m, 1H), 7.58- 7.53 (m, 1H), 7.49 (t, J = 7.9 Hz, 1H), 7.38 (d, J = 7.6 Hz, 1H), 4.35-4.23 (m, 2H), 3.11 (t, J = 5.9 Hz, 2H), 2.45 (t, J = 7.3 Hz, 2H), 1.48 (sextet, J = 7.4 Hz, 2H), 0.81 (t, J = 7.5 Hz, 3H) |
| 4-nitrophenyl 5- (difluoro(phenoxy (2-(pivaloylthio) ethoxy)phosphoryl) methyl)benzo[b] thiophene-2- carboxylate | 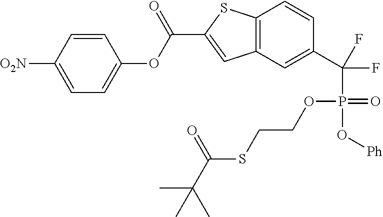 [SMILES: CC(C)(C)C(=O)SCCOP(=O)(Oc1ccccc1)C(F)(F)c1ccc2sc(C(=O)Oc3ccc([N+](=O)[O-])cc3)cc2c1] | | ¹H NMR (400 MHz, DMSO-d₆) δ 8.67 (s, 1H), 8.49-8.34 (m, 4H), 7.80 (d, J = 8.8 Hz, 1H), 7.70 (d, J = 9.0 Hz, 2H), 7.41 (t, J = 7.6 Hz, 2H), 7.30-7.15 (m, 3H), 4.33-4.23 (m, 2H), 3.12 (t, J = 5.6 Hz, 2H), 1.12 (s, 9H) |
| 4-nitrophenyl 5- (((4-cyanophenoxy) (2-(pivaloylthio) ethoxy)phosphoryl) difluoromethyl)benzo [b]thiophene-2- carboxylate | 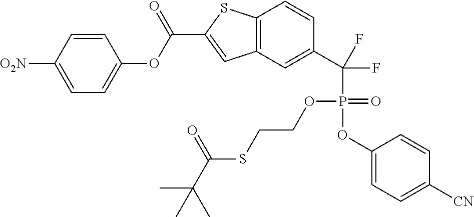 [SMILES: CC(C)(C)C(=O)SCCOP(=O)(Oc1ccc(C#N)cc1)C(F)(F)c1ccc2sc(C(=O)Oc3ccc([N+](=O)[O-])cc3)cc2c1] | | |
| 4-nitrophenyl 5- (((3- cyanophenoxy)(2- (pivaloylthio) ethoxy)phosphoryl) difluoromethyl) benzo[b]thiophene- 2-carboxylate | 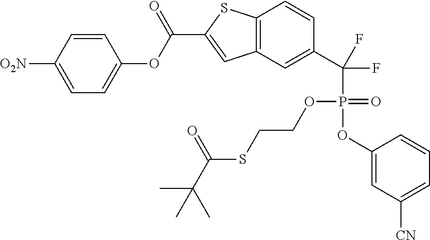 [SMILES: CC(C)(C)C(=O)SCCOP(=O)(Oc1cccc(C#N)c1)C(F)(F)c1ccc2sc(C(=O)Oc3ccc([N+](=O)[O-])cc3)cc2c1] | | ¹H NMR (400 MHz, DMSO-d₆) δ 8.66 (s, 1H), 8.46 (s, 1H), 8.42-8.34 (m, 3H), 7.83-7.67 (m, 5H), 7.66-7.59 (m, 1H), 7.58- 7.53 (m, 1H), 4.40-4.28 (m, 2H), 3.14 (t, J = 6.2 Hz, 2H), 1.12 (s, 9H) |
| 4-nitrophenyl 5- (((2- (butyrylthio) ethoxy)(3- cyanophenoxy) phosphoryl) difluoromethyl) benzo[b] thiophene-2- carboxylate | 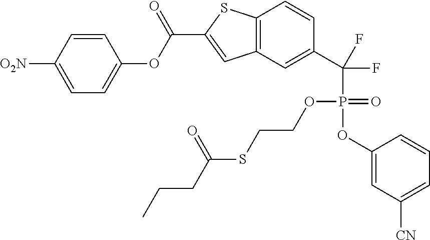 [SMILES: CCCC(=O)SCCOP(=O)(Oc1cccc(C#N)c1)C(F)(F)c1ccc2sc(C(=O)Oc3ccc([N+](=O)[O-])cc3)cc2c1] | 661.1 [M + H]⁺ | |
| 4-nitrophenyl 5- (difluoro((2-((3- methylbutanoyl) thio)ethoxy)(phenoxy) phosphoryl) methyl)benzo[b] thiophene-2- carboxylate | 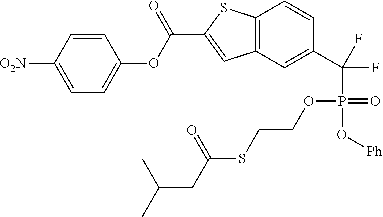 [SMILES: CC(C)CC(=O)SCCOP(=O)(Oc1ccccc1)C(F)(F)c1ccc2sc(C(=O)Oc3ccc([N+](=O)[O-])cc3)cc2c1] | | ¹H NMR (400 MHz, DMSO-d₆) δ 8.67 (s, 1H), 8.49-8.34 (m, 4H), 7.80 (d, J = 8.8 Hz, 1H), 7.70 (d, J = 9.0 Hz, 2H), 7.41 (t, J = 7.6 Hz, 2H), 7.30-7.15 (m, 3H), 4.33-4.23 (m, 2H), 3.12 (t, J = 5.6 Hz, 2H), 1.12 (s, 9H) |
| perfluorophenyl 5-(((2-((3-((tert- butyldimethylsilyl) oxy)-2,2- dimethylpropanoyl) thio)ethoxy) (phenoxy)phosphoryl) difluoromethyl) benzo[b]thiophene- 2-carboxylate | 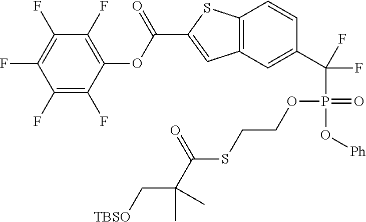 [SMILES: CC(C)(CO[Si](C)(C)C(C)(C)C)C(=O)SCCOP(=O)(Oc1ccccc1)C(F)(F)c1ccc2sc(C(=O)Oc3c(F)c(F)c(F)c(F)c3F)cc2c1] | 847.0 [M + Na]⁺ | |

### Method 2: One-Pot Acid Chloride Method for the Synthesis of Activated Linkers

Representative procedure for the synthesis of perfluorophenyl 5-((bis(4-((3-methylbutanoyl)thio)butoxy)phosphoryl)difluoromethyl)benzo[b]thiophene-2-carboxylate

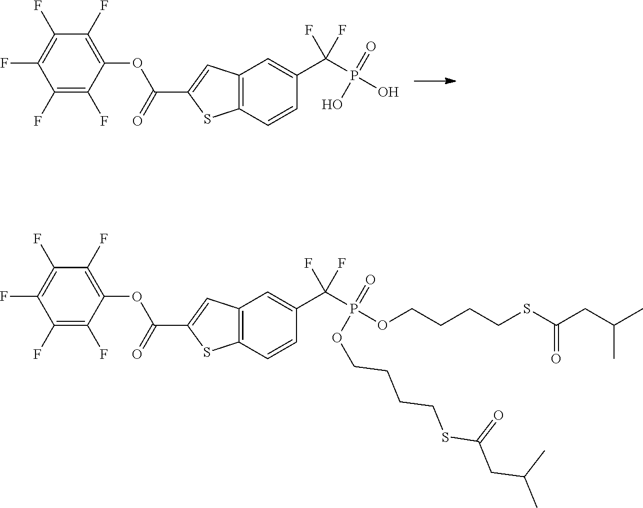 [SMILES: O=C(Oc1c(F)c(F)c(F)c(F)c1F)c1cc2cc(C(F)(F)P(=O)(O)O)ccc2s1 ; CC(C)CC(=O)SCCCCOP(=O)(OCCCCSC(=O)CC(C)C)C(F)(F)c1ccc2sc(C(=O)Oc3c(F)c(F)c(F)c(F)c3F)cc2c1]

To a cooled (0° C.) solution of 3 (200 mg, 0.42 mmol, 1.0 eq) in dry CH₂Cl₂ (15 mL) and catalytic DMF (3.2 μL, 42.1 μmol, 0.1 eq) was added in a dropwise manner oxalyl chloride (266 mg, 2.10 mmol, 5.0 eq). The reaction mixture was allowed to warm to 40° C. After stirring for 2 h, the reaction mixture was concentrated in vacuo and dried (to remove excess oxalyl chloride). The resulting solids were re-dissolved in anhydrous CH₂Cl₂ (5 mL) and cooled to 0° C. To the cooled solution was added S-(4-hydroxybutyl) 3-methylbutanethioate (239 mg, 1.26 mmol, 3.0 eq), DMAP (5.14 mg, 42.1 μmol, 0.1 eq) and a solution of N,N-diisopropylethylamine (217 mg, 1.68 mmol, 4.0 eq) in anhydrous CH₂Cl₂ (10 mL). The reaction mixture was allowed to warm to room temperature and stirred for additional 18 h. The reaction was quenched by adding H₂O (10 mL) and extracted with CH₂Cl₂ (3×10 mL). The organic layers were combined and washed with brine (20 mL), dried over anhydrous Na₂SO₄, and concentrated in vacuo. The residue was purified by flash column chromatography to afford perfluorophenyl 5-((bis(4-((3-methylbutanoyl)thio)butoxy)phosphoryl)difluoromethyl)benzo[b]thiophene-2-carboxylate (15.0 mg, 18.3 μmol, 4.4% yield). LCMS (ESI) m/z=819 [M+H]⁺.

The following intermediates in Table 9 were prepared using a similar protocol described above for synthesis of perfluorophenyl 5-((bis(4-((3-methylbutanoyl)thio)butoxy)phosphoryl)difluoromethyl)benzo[b]thiophene-2-carboxylate and utilizing the appropriate advanced intermediate(s) as starting material(s).

TABLE 9

| Name | Structure | LC-MS | NMR |
|---|---|---|---|
| 4-nitrophenyl 5- ((bis(2- (pivaloylthio)ethoxy) phosphoryl) difluoromethyl) benzo[b]thiophene- 2-carboxylate | 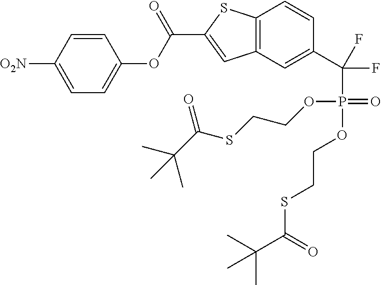 [SMILES: CC(C)(C)C(=O)SCCOP(=O)(OCCSC(=O)C(C)(C)C)C(F)(F)c1ccc2sc(C(=O)Oc3ccc([N+](=O)[O-])cc3)cc2c1] | | ¹H NMR (400 MHz, DMSO-d₆) δ 8.66 (s, 1H), 8.41-8.32 (m, 4H), 7.76- 7.65 (m, 3H), 4.22- 4.11 (m, 4H), 3.12 (t, J = 6.4 Hz, 4H), 1.16 (s, 18H) |
| perfluorophenyl 5- ((bis(2- (pivaloylthio)ethoxy) phosphoryl)methyl) benzo[b]thiophene- 2-carboxylate | 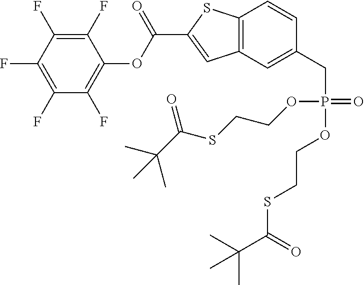 [SMILES: CC(C)(C)C(=O)SCCOP(=O)(Cc1ccc2sc(C(=O)Oc3c(F)c(F)c(F)c(F)c3F)cc2c1)OCCSC(=O)C(C)(C)C] | 749.0 [M + Na]⁺ | |

### Method 3: One-Pot Silver Salt Method for the Synthesis of Activated Linkers

Representative procedure for the synthesis of perfluorophenyl 5-((bis(((isopropoxycarbonyl)oxy)methoxy)phosphoryl)methyl)benzo[b]thiophene-2-carboxylate

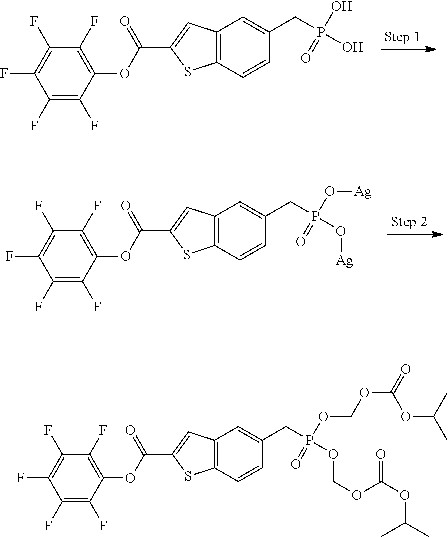

Step 1: Preparation of silver(I) ((2-((perfluorophenoxy)carbonyl)benzo[b]thiophen-5-yl)methyl)phosphonate

To a solution of ((2-((perfluorophenoxy)carbonyl)benzo[b]thiophen-5-yl)methyl)phosphonic acid (300 mg, 684 μmol, 1.0 eq) in a mixture of deionized H₂O (4 mL) and THF (2 mL) was added Amberlite IR120® resin (Na⁺ form) (1.5 g). The resulting mixture was stirred at room temperature for 1 h and the suspension was subsequently filtered. To the filtrate was added a solution of AgNO₃ (463 mg, 2.73 mmol, 4.0 eq) in deionized H₂O (2 mL) and the resulting mixture was stirred at room temperature for an additional 1 h. Formation of a white precipitate was observed and the solids were collected via filtration. The filter cake was then washed with cold H₂O (3×2 mL), and the solid was dried under reduced pressure to yield silver(I) ((2-((perfluorophenoxy)carbonyl)benzo[b]thiophen-5-yl)methyl)phosphonate as a dry powder. The silver salt was used without further purification.

Step 2: Preparation of perfluorophenyl 5-((bis(((isopropoxycarbonyl)oxy)methoxy)phosphoryl)methyl)benzo[b]thiophene-2-carboxylate

To a suspension of silver(I) ((2-((perfluorophenoxy)carbonyl)benzo[b]thiophen-5-yl)methyl)phosphonate was suspended in anhydrous toluene (10 mL) and iodomethyl 2-methylpropanoate (500 mg, 2.05 mmol, 3.0 eq) was added in a dropwise manner. After addition, the resulting mixture was stirred at room temperature for an additional 12 h. The reaction progress was monitored by LCMS, and after completion, the unreacted silver salt was recovered by filtration. The filtrate solution was concentrated in vacuo, and the resulting residue was purified by reverse phase chromatography [C18 column gradient elution water/acetonitrile=90% to 1%] to give perfluorophenyl 5-((bis(((isopropoxycarbonyl)oxy)methoxy)phosphoryl)methyl)benzo[b]thiophene-2-carboxylate (165 mg, 246 μmol, 36% yield) as a white solid. LCMS (ESI) m/z=671 [M+H]⁺; ¹H NMR (400 MHz, CDCl₃) δ 8.28 (s, 1H), 7.96-7.80 (m, 2H), 7.48 (d, J=8.5 Hz, 1H), 5.70-5.53 (m, 4H), 4.90 (dt, J=12.6, 6.2 Hz, 2H), 3.42 (d, J=22.2 Hz, 2H), 1.31 (d, J=6.2 Hz, 12H).

The following intermediates in Table 10 were prepared using a similar protocol described above for synthesis of perfluorophenyl 5-((bis(4-((3-methylbutanoyl)thio)butoxy)phosphoryl)difluoromethyl)benzo[b]thiophene-2-carboxylate and utilizing the appropriate advanced intermediate(s) as starting material(s).

TABLE 10

| Name | Structure | LCMS | NMR |
|---|---|---|---|
| perfluorophenyl 5- ((bis(2- (butyrylthio)ethoxy) phosphoryl)methyl) benzo[b]thiophene- 2-carboxylate | 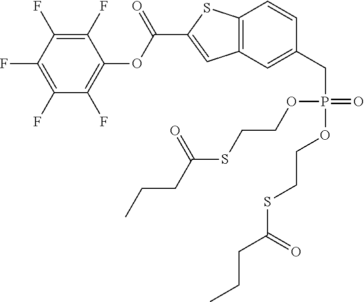 [SMILES: CCCC(=O)SCCOP(=O)(Cc1ccc2sc(C(=O)Oc3c(F)c(F)c(F)c(F)c3F)cc2c1)OCCSC(=O)CCC] | 699 [M + H]+ | ¹H NMR (400 MHz, CDCl₃) δ 8.31 (s, 1H), 7.96-7.80 (m, 2H), 7.50 (d, J = 8.3 Hz, 1H), 4.08- 4.03 (m, 4H), 3.33 (d, J_{HP} = 21.6 Hz, 2H), 3.08 (t, J = 6.5 Hz, 4H), 2.52 (t, J = 7.4 Hz, 4H), 1.72-1.63 (m, 4H), 0.94 (t, J = 7.4 Hz, 6H) |
| 4-nitrophenyl 5- ((bis(2- (butyrylthio)ethoxy) phosphoryl) difluoromethyl)benzo [b]thiophene-2- carboxylate | 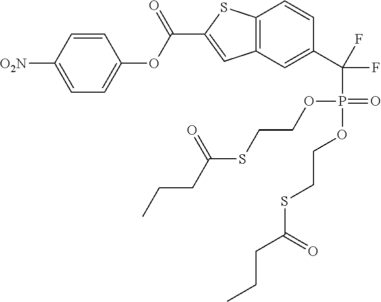 [SMILES: CCCC(=O)SCCOP(=O)(OCCSC(=O)CCC)C(F)(F)c1ccc2sc(C(=O)Oc3ccc([N+](=O)[O-])cc3)cc2c1] | 690 [M + H]⁺ | ¹H NMR (400 MHz, CDCl₃) δ 8.35-8.34 (m, 3H), 8.23 (s, 1H), 8.03 (d, J = 8.4 Hz, 1H), 7.76 (d, J = 8.7 Hz, 1H), 7.48 (d, J = 9.0 Hz, 2H), 4.29-4.19 (m, 4H), 3.27-3.03 (m, 4H), 2.54 (t, J = 7.4 Hz, 4H), 1.75-1.63 (m, 4H), 0.95 (t, J = 7.4 Hz, 6H) |
| perfluorophenyl 5- ((bis(2-((3- methylbutanoyl) thio)ethoxy) phosphoryl) difluoromethyl) benzo[b]thiophene- 2-carboxylate | 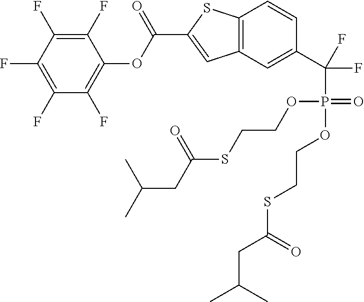 [SMILES: CC(C)CC(=O)SCCOP(=O)(OCCSC(=O)CC(C)C)C(F)(F)c1ccc2sc(C(=O)Oc3c(F)c(F)c(F)c(F)c3F)cc2c1] | | ¹H NMR (400 MHz, CDCl₃) δ 8.41 (s, 1H), 8.24 (s, 1H), 8.03 (d, J = 8.6 Hz, 1H), 7.78 (d, J = 8.7 Hz, 1H), 4.34-4.08 (m, 4H), 3.25-3.05 (m, 4H), 2.43 (d, J = 7.1 Hz, 4H), 2.14 (dt, J = 13.7, 6.8 Hz, 2H), 0.95 (s, 6H), 0.94 (s, 6H) |
| ((((2- ((perfluorophenoxy) carbonyl) benzo[b]thiophen-5- yl)methyl)phosphoryl) bis(oxy))bis(methylene) bis(2,2- dimethylpropanoate) | 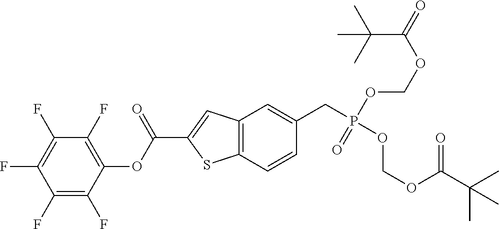 [SMILES: CC(C)(C)C(=O)OCOP(=O)(Cc1ccc2sc(C(=O)Oc3c(F)c(F)c(F)c(F)c3F)cc2c1)OCOC(=O)C(C)(C)C] | 689 [M + H]⁺ | ¹H NMR (400 MHz, CDCl₃) δ 8.28 (s, 1H), 7.90-7.86 (m, 2H), 7.50- 7.45 (m, 1H), 5.68-5.56 (m, 4H), 3.38 (m, 2H), 1.19 (s, 18H) |
| (((difluoro(7-((4- nitrophenoxy)carbonyl) naphthalen-2- yl)methyl)phosphoryl) bis(oxy))bis(methylene) bis(2,2- dimethylpropanoate) | 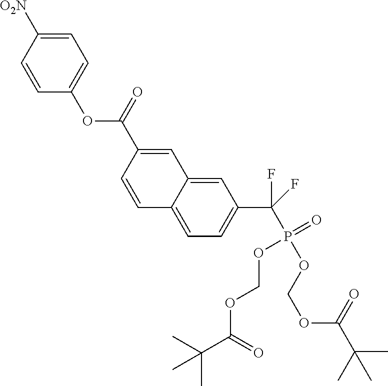 [SMILES: CC(C)(C)C(=O)OCOP(=O)(OCOC(=O)C(C)(C)C)C(F)(F)c1ccc2ccc(C(=O)Oc3ccc([N+](=O)[O-])cc3)cc2c1] | 674.1 [M + H]⁺ | ¹H NMR (400 MHz, CDCl₃) δ 8.90 (s, 1H), 8.40-8.34 (m, 2H), 8.34- 8.26 (m, 2H), 8.04 (d, J = 8.4 Hz, 2H), 7.85 (d, J = 8.4 Hz, 1H), 7.52-7.47 (m, 2H), 5.81-5.66 (m, 4H), 1.21 (s, 18H) |
| perfluorophenyl 5- ((bis(2-((3-((tert- butyldimethylsilyl) oxy)-3- methylbutanoyl) thio)ethoxy)phosphoryl) difluoromethyl)benzo [b]thiophene- 2-carboxylate | 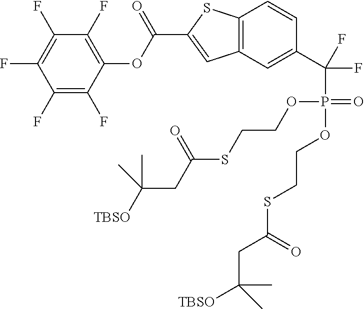 [SMILES: CC(C)(CC(=O)SCCOP(=O)(OCCSC(=O)CC(C)(C)O[Si](C)(C)C(C)(C)C)C(F)(F)c1ccc2sc(C(=O)Oc3c(F)c(F)c(F)c(F)c3F)cc2c1)O[Si](C)(C)C(C)(C)C] | | ¹H NMR (400 MHz, CDCl₃) δ 8.39-8.30 (m, 3H), 8.22 (s, 1H), 8.01 (d, J = 8.6 Hz, 1H), 7.75 (d, J = 8.5 Hz, 1H), 7.47 (d, J = 8.9 Hz, 2H), 4.25-4.13 (m, 4H), 3.57 (s, 4H), 3.16- 3.05 (m, 4H), 1.18 (s, 12H), 0.84 (s, 18H), 0.00 (s, 12H) |
| perfluorophenyl 5- ((bis(2-((3,3,3- trifluoro-2,2- dimethylpropanoyl) thio)ethoxy)phosphoryl) difluoromethyl) benzo[b]thiophene- 2-carboxylate | 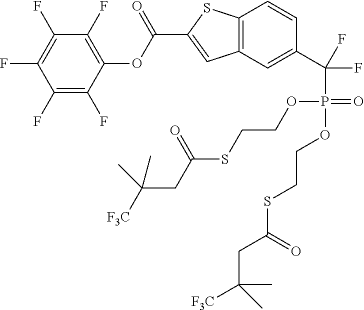 [SMILES: CC(C)(CC(=O)SCCOP(=O)(OCCSC(=O)CC(C)(C)C(F)(F)F)C(F)(F)c1ccc2sc(C(=O)Oc3c(F)c(F)c(F)c(F)c3F)cc2c1)C(F)(F)F] | 893.2 [M + Na]⁺ | |
| perfluorophenyl 7- ((bis(2-((3- methylbutanoyl)thio) ethoxy)phosphoryl) difluoromethyl)- 2-naphthoate | 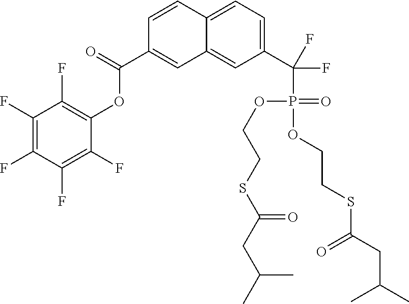 [SMILES: CC(C)CC(=O)SCCOP(=O)(OCCSC(=O)CC(C)C)C(F)(F)c1ccc2ccc(C(=O)Oc3c(F)c(F)c(F)c(F)c3F)cc2c1] | 799 [M + Na]⁺ | |
| perfluorophenyl 5- ((bis (((isopropoxycarbonyl) oxy)methoxy) phosphoryl) methyl)benzo[b] thiophene- 2-carboxylate | 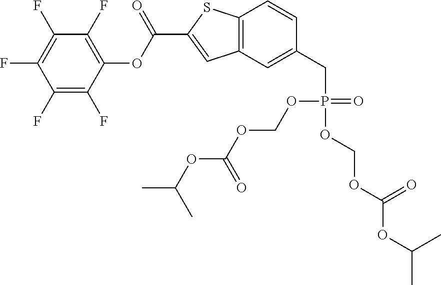 [SMILES: CC(C)OC(=O)OCOP(=O)(Cc1ccc2sc(C(=O)Oc3c(F)c(F)c(F)c(F)c3F)cc2c1)OCOC(=O)OC(C)C] | | ¹H NMR (400 MHz, CDCl₃) δ 8.28 (s, 1H), 7.96-7.80 (m, 2H), 7.48 (d, J = 8.5 Hz, 1H), 5.70- 5.53 (m, 4H), 4.90 (dt, J = 12.6, 6.2 Hz, 2H), 3.42 (d, J = 22.2 Hz, 2H), 1.31 (d, J = 6.2 Hz, 12H) |
| perfluorophenyl 5- ((bis(2-((3-((tert- butyldimethylsilyl) oxy)-3- methylbutanoyl)thio) ethoxy)phosphoryl) methyl)benzo[b] thiophene-2- carboxylate | 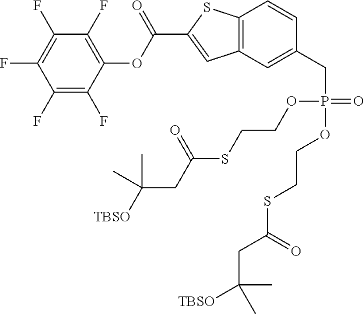 [SMILES: CC(C)(CC(=O)SCCOP(=O)(Cc1ccc2sc(C(=O)Oc3c(F)c(F)c(F)c(F)c3F)cc2c1)OCCSC(=O)CC(C)(C)O[Si](C)(C)C(C)(C)C)O[Si](C)(C)C(C)(C)C] | | ¹H NMR (400 MHz, CDCl₃) δ 8.30 (s, 1H), 7.95-7.80 (m, 2H), 7.50 (d, J = 8.4 Hz, 1H), 4.01 (dd, J = 14.7, 7.0 Hz, 4H), 3.58 (d, J= 10.6 Hz, 4H), 3.32 (d, J = 21.6 Hz, 2H), 3.04 (t, J = 6.7 Hz, 4H), 1.18 (d, J = 8.5 Hz, 12H), 0.84 (s, 18H), 0.00 (d, J = 3.5 Hz, 12H) |

(R)- or (S)-5-((bis((pivaloyloxy)methoxy)phosphoryl)fluoromethyl)benzo[b]thiophene-2-carboxylic acid

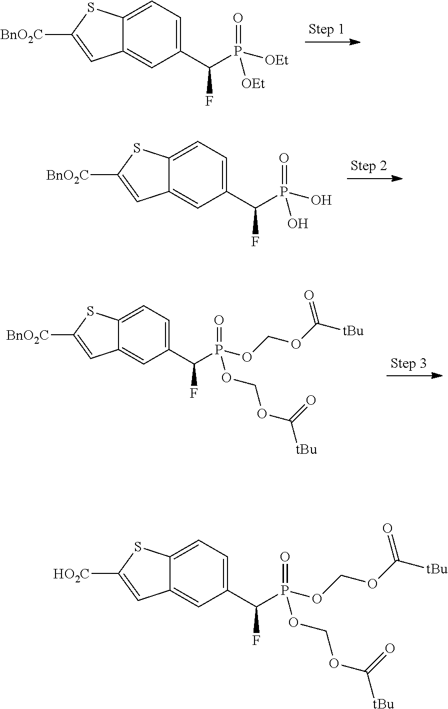

Step 1: Preparation of (R)- or (S)-((2-((benzyloxy)carbonyl)benzo[b]thiophen-5-yl)fluoromethyl)phosphonic acid

To a cooled (0° C.) solution of benzyl (R)- or (S)-5-((diethoxyphosphoryl)fluoromethyl)benzo[b]thiophene-2-carboxylate (Peak 1) (205 mg, 0.4697 mmol) in CH₂Cl₂ (8.0 mL) was added BSTFA (746 μL, 2.81 mmol) followed by a 1.0 M solution of trimethylsilyl iodide (1.87 mL, 1.87 mmol) in CH₂Cl₂. After stirring for 1 h, a mixture of acetonitrile (0.66 mL), water (0.33 mL) and 0.1% TFA was added. The solvent was removed under reduced pressure at 0° C. The crude residue was purified by reverse phase chromatography (C18 cartridge eluting with 5-40% acetonitrile in water) to give (R)- or (S)-((2-((benzyloxy)carbonyl)benzo[b]thiophen-5-yl)fluoromethyl)phosphonic acid (163 mg, 0.4285 mmol) as a white solid. ¹H NMR (400 MHz, DMSO-d₆) δ 8.33 (s, 1H), 8.11-8.06 (m, 2H), 7.58 (d, J=8.6 Hz, 1H), 7.51-7.47 (m, 2H), 7.45-7.33 (m, 3H), 5.84 (dd, J=44.6, 8.3 Hz, 1H), 5.40 (s, 2H).

Step 2: Preparation of (R)- or (S)-((2-((benzyloxy)carbonyl)benzo[b]thiophen-5-yl)fluoromethyl)phosphonic acid

[The following reaction was conducted in foiled covered vessel and in the absence of ambient light]: To a suspension of (R)- or (S)-((2-((benzyloxy)carbonyl)benzo[b]thiophen-5-yl)fluoromethyl)phosphonic acid (163 mg, 0.4285 mmol, 1 eq) in water (5 mL) was added a solution of aqueous sodium hydroxide (34.2 mg, 857 μmol, 2 eq) in water (2 mL). To the yellow solution was added silver(I) nitrate (181 mg, 1.07 mmol, 2.5 eq) and the resulting off-white suspension was stirred for 1.5 h at room temperature. The suspension was cooled to 0° C., filtered, and dried under high vacuum. The solids were re-suspended in acetonitrile, concentrated under reduced pressure, and further dried under high vacuum (3 h). The resulting dark yellow powder was suspended in toluene (10 mL) and iodomethyl 2,2-dimethylpropanoate (191 μL, 1.28 mmol, 3 eq) was added. After stirring for 20 h, the reaction mixture was stirred for 20 h at room temperature. The reaction mixture was filtered and rinsed with toluene. The filtrate was concentrated under reduced pressure. The crude residue was purified (C18 cartridge eluting with 5-100% acetonitrile in water) to give (R)- or (S)-((((2-((benzyloxy)carbonyl)benzo[b]thiophen-5-yl)fluoromethyl)phosphoryl)bis(oxy))bis(methylene) bis(2,2-dimethylpropanoate) (122 mg, 0.2004 mmol, 46.9%) as a clear oil. ¹H NMR (400 MHz, CDCl₃) δ 8.11 (s, 1H), 8.01-7.99 (m, 1H), 7.92 (d, J=8.3 Hz, 1H), 7.59 (d, J=8.3 Hz, 1H), 7.52-7.47 (m, 2H), 7.46-7.36 (m, 3H), 8.87 (dd, J=44.3, 7.5 Hz, 1H), 5.72-5.62 (m, 4H), 5.43 (s, 2H), 1.21 (s, 18H).

Step 3: Preparation of (R)- or (S)-5-((bis((pivaloyloxy)methoxy)phosphoryl)fluoromethyl)benzo[b]thiophene-2-carboxylic acid

To a solution of (R)-((((2-((benzyloxy)carbonyl)benzo[b]thiophen-5-yl)fluoromethyl)phosphoryl)bis(oxy))bis(methylene) bis(2,2-dimethylpropanoate) (122 mg, 0.200 mmol, 1 eq) in THF (10 mL) under N₂ (g) was added 10% Pd/C (50% wet, 120 mg, 0.1127 mmol, 0.56 eq). To the suspension was bubbled H₂ (g) for 5 min. The reaction mixture was stirred under H₂ (g) (1 atm) at room temperature. After stirring for 22 h, the reaction mixture was purged with N₂ (g) and filtered over Celite®. The filter pad was washed with THF and concentrated under reduced pressure to give (R)-5-((bis((pivaloyloxy)methoxy)phosphoryl)fluoromethyl)benzo[b]thiophene-2-carboxylic acid (0.200 mmol, 99.9%) as a thick clear oil. LC-MS (ESI) m/z [M+H]⁺=519.1.

The following intermediates in Table 11 were prepared using a similar protocol described above for synthesis of (R)- or (S)-5-((bis((pivaloyloxy)methoxy)phosphoryl)fluoromethyl)benzo[b]thiophene-2-carboxylic acid and utilizing the appropriate advanced intermediate(s) as starting material(s). The absolute configuration of the starting material, (R)- or (S)-((2-((benzyloxy)carbonyl)benzo[b]thiophen-5-yl)fluoromethyl)phosphonic acid was not determined.

TABLE 11

| Name | Structure | LCMS | NMR |
|---|---|---|---|
| (R)- or (S)-5- ((bis((pivaloyloxy) methoxy)phosphoryl) fluoromethyl)benzo[b] thiophene-2- carboxylic acid | 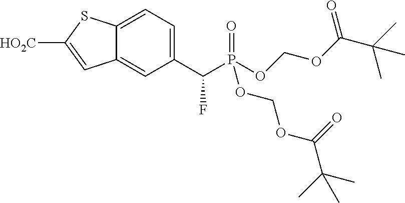 [SMILES: CC(C)(C)C(=O)OCOP(=O)(OCOC(=O)C(C)(C)C)[C@H](F)c1ccc2sc(C(=O)O)cc2c1] | 519.1 [M + H]⁺ | |
| | Or | | |
| | 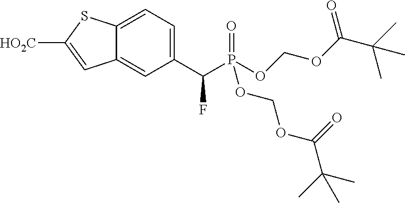 [SMILES: CC(C)(C)C(=O)OCOP(=O)(OCOC(=O)C(C)(C)C)[C@@H](F)c1ccc2sc(C(=O)O)cc2c1] | | |
| (R)- or (S)-5- ((bis (((isopropoxycarbonyl) oxy)methoxy) phosphoryl) fluoromethyl) benzo[b]thiophene-2-carboxylic acid | 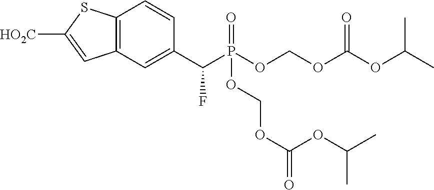 | 689 [M + H]⁺ | ¹H NMR (400 MHZ, CDCl₃) δ 8.35 (s, 1H), 8.09 (s, 1H), 7.97 (d, J = 8.5 Hz, 1H), 7.65 (d, J = 8.6 Hz, 1H), 5.93 (dd, J = 44.2, 7.3 Hz, 1H), 5.73-5.54 (m, 4H), 4.97-4.84 (m, 2H), 1.33-1.29 (m, 12H) |
| | Or | | |
| | 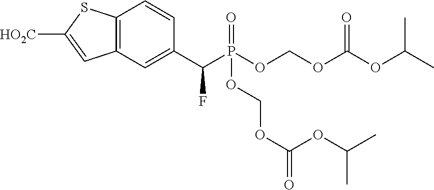 [SMILES: CC(C)OC(=O)OCOP(=O)(OCOC(=O)OC(C)C)[C@@H](F)c1ccc2sc(C(=O)O)cc2c1] | | |

### Method 4: Stepwise Silver Salt Method for the Synthesis of Mixed Linkers

Representative procedure for the perfluorophenyl 5-(((2-(butyrylthio)ethoxy)(2-(pivaloylthio)ethoxy)phosphoryl)difluoromethyl)benzo[b]thiophene-2-carboxylate

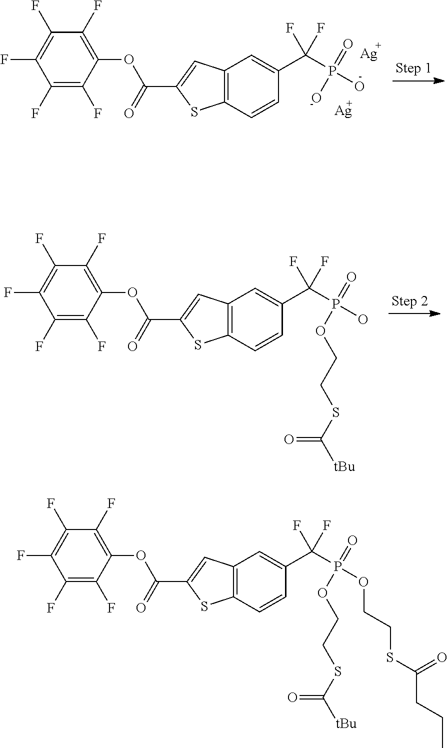

Step 1: Preparation of perfluorophenyl 5-(difluoro(hydroxy(2-(pivaloylthio)ethoxy)phosphoryl)methyl)benzo[b]thiophene-2-carboxylate
Silver(I) (difluoro(2-((perfluorophenoxy)carbonyl)benzo[b]thiophen-5-yl)methyl)phosphonate was synthesized starting from (difluoro(2-((perfluorophenoxy)carbonyl)benzo[b]thiophen-5-yl)methyl)phosphonic acid using the method described in Step 1, Method 3 for the synthesis of silver(I) ((2-((perfluorophenoxy)carbonyl)benzo[b]thiophen-5-yl)methyl)phosphonate

To a suspension of silver(I) ((2-((perfluorophenoxy)carbonyl)benzo[b]thiophen-5-yl)methyl)phosphonate (647 mg, 940 μmol, 1.0 eq) in anhydrous toluene (10 mL) was added in a dropwise manner S-(2-iodoethyl) 2,2-dimethylpropanethioate (310 mg, 1.14 mmol, 1.2 eq). After complete addition of the alcohol, the resulting mixture was stirred at room temperature for an additional 12 h. The heterogeneous mixture was filtered, and the filtrate was concentrated in vacuo. The resulting residue was purified by reverse phase chromatography to give perfluorophenyl 5-(difluoro(hydroxy(2-(pivaloylthio)ethoxy)phosphoryl)methyl)benzo[b]thiophene-2-carboxylate (240 mg, 388 μmol, 41% yield). LCMS (ESI) m/z=617 [M−H]⁻.

Step 2: Preparation of perfluorophenyl 5-(((2-(butyrylthio)ethoxy)(2-(pivaloylthio)ethoxy)phosphoryl)difluoromethyl)benzo[b]thiophene-2-carboxylate

Perfluorophenyl 5-(((2-(butyrylthio)ethoxy)(2-(pivaloylthio)ethoxy)phosphoryl)difluoromethyl)benzo[b]thiophene-2-carboxylate was synthesized using similar protocol outlined above. Starting with perfluorophenyl 5-(difluoro(hydroxy(2-(pivaloylthio)ethoxy)phosphoryl)methyl)benzo[b]thiophene-2-carboxylate (190 mg, 307 μmol, 1.0 eq), AgNO₃ (207 mg, 1.22 mmol, 4.0 eq), and S-(2-iodoethyl) butanethioate (94.9 mg, 368 μmol, 1.2 eq) produced perfluorophenyl 5-(((2-(butyrylthio)ethoxy)(2-(pivaloylthio)ethoxy)phosphoryl)difluoromethyl)benzo[b]thiophene-2-carboxylate (55.0 mg, 73.4 μmol, 24%) as a white solid. LCMS (ESI) m/z=749 [M+H]⁺.

The following intermediates in Table 12 were prepared using a similar protocol described above for synthesis of perfluorophenyl 5-(((2-(butyrylthio)ethoxy)(2-(pivaloylthio)ethoxy)phosphoryl)difluoromethyl)benzo[b]thiophene-2-carboxylate and utilizing the appropriate advanced intermediate(s) as starting material(s).

TABLE 12

| Name | Structure | LCMS | NMR |
|---|---|---|---|
| perfluorophenyl 5- (difluoro ((((isopropoxycarbonyl) oxy)methoxy)(2-((3- methylbutanoyl)thio) ethoxy)phosphoryl) methyl)benzo[b] thiophene-2- carboxylate | 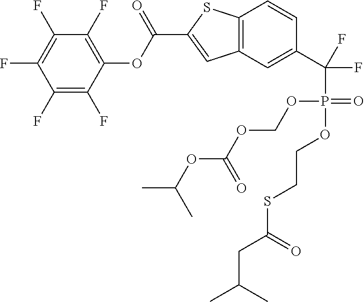 [SMILES: CC(C)CC(=O)SCCOP(=O)(OCOC(=O)OC(C)C)C(F)(F)c1ccc2sc(C(=O)Oc3c(F)c(F)c(F)c(F)c3F)cc2c1] | 757 [M + Na]⁺ | ¹H NMR (400 MHZ, CDCl₃) δ 8.40 (s, 1H), 8.24 (s, 1H), 8.03 (d, J = 8.6 Hz, 1H), 7.77 (d, J = 8.7 Hz, 1H), 5.69 (ddd, J = 17.8, 12.2, 5.2 Hz, 2H), 4.93 (dt, J = 12.5, 6.3 Hz, 1H), 4.34- 4.14 (m, 2H), 3.14 (td, J = 6.6, 3.0 Hz, 2H), 2.42 (d, J = 7.1 Hz, 2H), 2.13 (dt, J = 13.7, 6.8 Hz, 1H), 1.33 (s, 3H), 1.31 (s, 3H), 0.95 (s, 3H), 0.93 (s, 3H) |

Preparation of 5-(((((S)-1-isopropoxy-1-oxopropan-2-yl)amino)(phenoxy)phosphoryl)methyl)benzo[b]thiophene-2-carboxylic acid

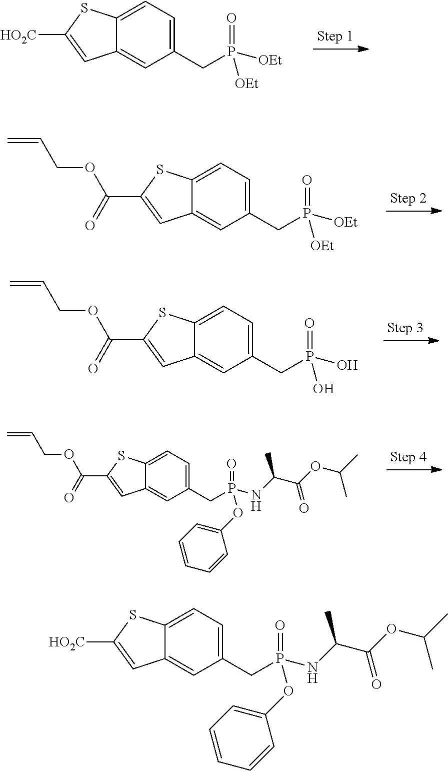

Step 1: Preparation of allyl 5-((diethoxyphosphoryl)methyl)benzo[b]thiophene-2-carboxylate

To a suspension of 5-((diethoxyphosphoryl)methyl)benzo[b]thiophene-2-carboxylic acid (1.0 g, 3.0 mmol, 1.0 eq) and K₂CO₃ (839 mg, 6.1 mmol, 2.0 eq) in DMF (20 mL) was added 3-bromoprop-1-ene (440 mg, 3.6 mmol, 1.2 eq). The mixture was stirred at room temperature for 14 h and poured over water (30 mL). The mixture was extracted with EtOAc (25 mL×3). The combined organic layers were dried over Na₂SO₄, filtered, and concentrated under reduced pressure, the residue was purified by column chromatography to give allyl 5-((diethoxyphosphoryl)methyl)benzo[b]thiophene-2-carboxylate (0.980 g, 2.7 mmol, 88% yield) as a light-yellow solid. LCMS (ESI) m/z=369 [M+H]⁺; ¹H NMR (400 MHz, CDCl₃) δ 8.04 (s, 1H), 7.84-7.77 (m, 2H), 7.45-7.37 (m, 1H), 6.18-5.94 (m, 1H), 5.49-5.39 (m, 1H), 5.36-5.28 (m, 1H), 4.87-4.83 (m, 2H), 4.08-3.97 (m, 4H), 3.27 (d, J=21.4 Hz, 2H), 1.25 (t, J=7.1 Hz, 6H).

Step 2: Preparation of ((2-((allyloxy)carbonyl)benzo[b]thiophen-5-yl)methyl)phosphonic acid

To a solution of allyl 5-((diethoxyphosphoryl)methyl)benzo[b]thiophene-2-carboxylate (980 mg, 2.7 mmol, 1.0 eq) in CH₂Cl₂ (15 mL) was added bromotrimethylsilane (3 mL). The mixture was stirred at room temperature for 14 h and subsequently concentrated under reduced pressure. The resulting residue was triturated with H₂O (5 mL) and the resulting precipitates were filtered. The filter cake was washed with H₂O (5 mL×2) and dried under reduced pressure to give ((2-((allyloxy)carbonyl)benzo[b]thiophen-5-yl)methyl)phosphonic acid (0.710 g, 2.3 mmol, 86% yield) as a white solid. LCMS (ESI) m/z=313 [M+H]⁺; ¹H NMR (400 MHz, DMSO-d₆) δ 8.20 (s, 1H), 7.96 (d, J=8.4 Hz, 1H), 7.92-7.83 (m, 1H), 7.50-7.38 (m, 1H), 6.12-5.98 (m, 1H), 5.46-5.38 (m, 1H), 5.32-5.27 (m, 1H), 4.85-4.80 (m, 2H), 3.08 (d, J=21.2 Hz, 2H).

Step 3: Preparation of allyl 5-(((((S)-1-isopropoxy-1-oxopropan-2-yl)amino)(phenoxy)phosphoryl)methyl)benzo[b]thiophene-2-carboxylate

To a cooled (0° C.) solution (under a constant stream of N₂ (g)) of ((2-((allyloxy)carbonyl)benzo[b]thiophen-5-yl)methyl)phosphonic acid (2.80 g, 8.96 mmol, 1 eq) and catalytic DMF (1 drop) in dry CH₂Cl₂ (50 mL) was added oxalyl chloride (3.40 g, 26.8 mmol, 3 eq). After effervescence of gas ceased, the mixture was warmed at 40° C. After 2 h, the mixture was cooled to room temperature and concentrated in vacuo to give yellow solids. The solids were subsequently diluted CH₂Cl₂ (50 mL) and cooled to 0° C. To the cooled solution was added phenol (0.843 g, 8.96 mmol, 1 eq) and Et₃N (4.53 g, 44.8 mmol, 5 eq). After complete addition, the mixture was warmed to room temperature and stirred for 1 h, followed by introduction of propan-2-yl (2S)-2-aminopropanoate (1.75 g, 13.4 mmol, 1.5 eq) to the mixture. After stirring for an additional 2 h, the mixture was concentrated to dryness. The residue was purified by C18 column (elution 50%-80% acetonitrile in water) to give allyl 5-(((((S)-1-isopropoxy-1-oxopropan-2-yl)amino)(phenoxy)phosphoryl)methyl)benzo[b]thiophene-2-carboxylate (2.23 g, 4.44 mmol, 49.6% yield) as white solids. LCMS (ESI) m/z=502.0 [M+H]⁺; ¹H NMR (400 MHz, CDCl₃) δ 8.05 (d, J=6.9 Hz, 1H), 7.91-7.80 (m, 2H), 7.53-7.43 (m, 1H), 7.29 (d, J=8.1 Hz, 2H), 7.18-7.09 (m, 3H), 6.05 (ddd, J=16.1, 10.9, 5.6 Hz, 1H), 5.48-5.40 (m, 1H), 5.32 (dd, J=10.4, 1.2 Hz, 1H), 4.98-4.87 (m, 1H), 4.85 (d, J=5.7 Hz, 2H), 4.04-3.85 (m, 1H), 3.44 (dd, J=20.7, 14.1 Hz, 2H), 3.12 (t, J=10.9 Hz, 1H), 1.21-1.10 (m, 9H).

Step 4: Preparation of 5-(((((S)-1-isopropoxy-1-oxopropan-2-yl)amino)(phenoxy)phosphoryl)methyl)benzo[b]thiophene-2-carboxylic acid

A solution of allyl 5-(((((S)-1-isopropoxy-1-oxopropan-2-yl)amino)(phenoxy)phosphoryl)methyl)benzo[b]thiophene-2-carboxylate (90 mg, 0.1794 mmol, 1 eq), pyrrolidine (12.7 mg, 179 μmol, 1 eq), Pd(PPh₃)₄ (10.3 mg, 8.97 μmol, 0.05 eq) in CH₂Cl₂ (5 mL) was stirred under N₂ (g). After 2 h, the reaction was concentrated in vacuo. The residue was purified by C18 column (elution 30%-70% acetonitrile in water) to yield 5-(((((S)-1-isopropoxy-1-oxopropan-2-yl)amino)(phenoxy)phosphoryl)methyl)benzo[b]thiophene-2-carboxylic acid (64.0 mg, 0.1386 mmol, 77.4% yield) as white solids. LCMS (ESI) m/z=462.1 [M+H]⁺.

The following intermediates in Table 13 were prepared using the described above for synthesis of 5-(((((S)-1-isopropoxy-1-oxopropan-2-yl)amino)(phenoxy)phosphoryl)methyl)benzo[b]thiophene-2-carboxylic acid and utilizing the appropriate utilizing the appropriate starting materials and modifications.

TABLE 13

| Name | Structure | LCMS |
|---|---|---|
| 5-(fluoro((((S)-1-isopropoxy-1-oxopropan-2- yl)amino)(phenoxy)phosphoryl)methyl)benzo [b]thiophene-2-carboxylic acid | 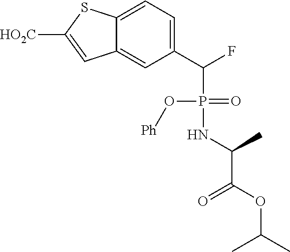 [SMILES: CC(C)OC(=O)[C@H](C)NP(=O)(Oc1ccccc1)C(F)c1ccc2sc(C(=O)O)cc2c1] | 480.1 [M + H]⁺ |
| 5-(((((S)-1-isopropoxy-1-oxopropan-2- yl)amino)(2,2,2- trifluoroethoxy)phosphoryl)methyl)benzo[b] thiophene-2-carboxylic acid | 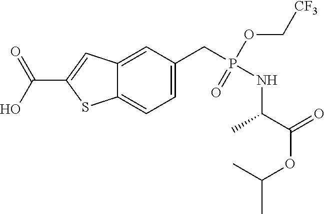 [SMILES: CC(C)OC(=O)[C@H](C)NP(=O)(Cc1ccc2sc(C(=O)O)cc2c1)OCC(F)(F)F] | 468.2 [M + H]⁺ |
| 5-(((4-chlorophenoxy)(((S)-1-isopropoxy-1- oxopropan-2- yl)amino)phosphoryl)methyl)benzo[b]thiophene- 2-carboxylic acid | 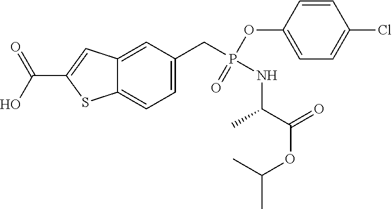 [SMILES: CC(C)OC(=O)[C@H](C)NP(=O)(Cc1ccc2sc(C(=O)O)cc2c1)Oc1ccc(Cl)cc1] | 496.2 [M + H]⁺ |
| 5-(((2-chlorophenoxy)(((S)-1-isopropoxy-1- oxopropan-2- yl)amino)phosphoryl)methyl)benzo[b]thiophene- 2-carboxylic acid | 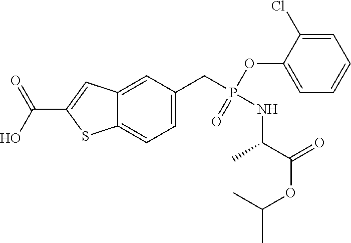 [SMILES: CC(C)OC(=O)[C@H](C)NP(=O)(Cc1ccc2sc(C(=O)O)cc2c1)Oc1ccccc1Cl] | 496.2 [M + H]⁺ |

### Synthetic Scheme:

Synthesis of 5-[1-(diethoxyphosphoryl)-2-hydroxyethyl]-1-benzothiophene-2-carboxylic acid

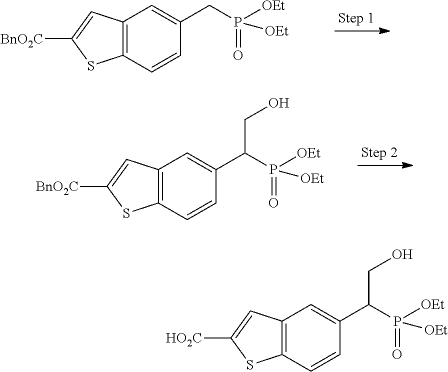

Step 1: Preparation of benzyl 5-[1-(diethoxyphosphoryl)-2-hydroxyethyl]-1-benzothiophene-2-carboxylate

To a solution of benzyl 5-[(diethoxyphosphoryl)methyl]-1-benzothiophene-2-carboxylate (200 mg, 0.4779 mmol, 1 eq) in tetrahydrofuran (10 mL) at −78° C. was added a solution of sodium bis(trimethylsilylamide) (1 M in THF) (1.43 mL, 1.43 mmol, 3.0 eq) dropwise. The reaction was stirred for 5 min. at −78° C. then 1H-benzotriazole-1-methanol (142 mg, 0.955 mmol, 2 eq) was added in one portion. The reaction was stirred at −78° C. for 2 h. The reaction mixture was quenched with saturated ammonium chloride aqueous solution (10 mL) at −78° C. then the ice-bath was removed. The product was extracted with EtOAc (3×30 mL). The combined extracts were dried with anhydrous sodium sulfate, filtered, and concentrated under reduced pressure. The crude residue was purified by reverse phase chromatography on a 50 g C₁₈ cartridge eluting with a gradient of 5-80% MeCN in water (with 0.1% formic acid) to give benzyl 5-[1-(diethoxyphosphoryl)-2-hydroxyethyl]-1-benzothiophene-2-carboxylate (24 mg, 0.05351 mmol, 11.2%) as a clear thick oil. LCMS: m/z=449.2 [M+H]⁺; ¹H NMR (400 MHz, DMSO-d₆) δ 8.27 (s, 1H), 8.02 (d, J=8.5 Hz, 1H), 7.99 (s, 1H), 5.40 (s, 2H), 4.87 (t, J=5.5 Hz, 1H), 4.07-3.94 (m, 3H), 3.93-3.71 (m, 3H), 3.52-3.41 (m, 1H), 1.21 (t, J=7.3 Hz, 3H), 1.03 (t, J=7.3 Hz, 3H).

Step 2: Preparation of 5-[1-(diethoxyphosphoryl)-2-hydroxyethyl]-1-benzothiophene-2-carboxylic acid

A mixture of 10% palladium on carbon (50% wet) (80 mg, 0.03758 mmol, 0.163 eq) and benzyl 5-[1-(diethoxyphosphoryl)-2-hydroxyethyl]-1-benzothiophene-2-carboxylatebenzyl 5-[1-(diethoxyphosphoryl)-2-hydroxyethyl]-1-benzothiophene-2-carboxylate (38 mg, 0.08473 mmol, 1 eq) in tetrahydrofuran (8 mL) was degassed with nitrogen for 5 min. Hydrogen was bubbled for 5 min. then the reaction was allowed to stir at room temperature under hydrogen (1 atm) for 20 h. The reaction was filtered over Celite and eluted with MeOH. The filtrate was concentrated under reduced pressure. The crude residue was purified by reverse phase chromatography on a 50 g C₁₈ cartridge eluting with 5-80% MeCN in water (containing 0.1% formic acid) then concentrated under reduced pressure and freeze dried to give 5-[1-(diethoxyphosphoryl)-2-hydroxyethyl]-1-benzothiophene-2-carboxylic acid (8.00 mg, 0.02232 mmol, 26.4%) as a white solid. LCMS: m/z=359.0 [M+H]⁺; ¹H NMR (400 MHz, DMSO-d₆) δ 8.02-7.92 (m, 2H), 7.90 (s, 1H), 7.48-7.42 (m, 1H), 4.92-4.79 (m, 1H), 4.08-3.69 (m, 6H), 3.52-3.39 (m, 1H), 1.21 (t, J=7.2 Hz, 3H), 1.03 (t, J=7.3 Hz, 3H).

Synthesis of 5-(1-(diethoxyphosphoryl)ethyl)benzo[b]thiophene-2-carboxylic acid and 5-(2-(diethoxyphosphoryl)propan-2-yl)benzo[b]thiophene-2-carboxylic acid

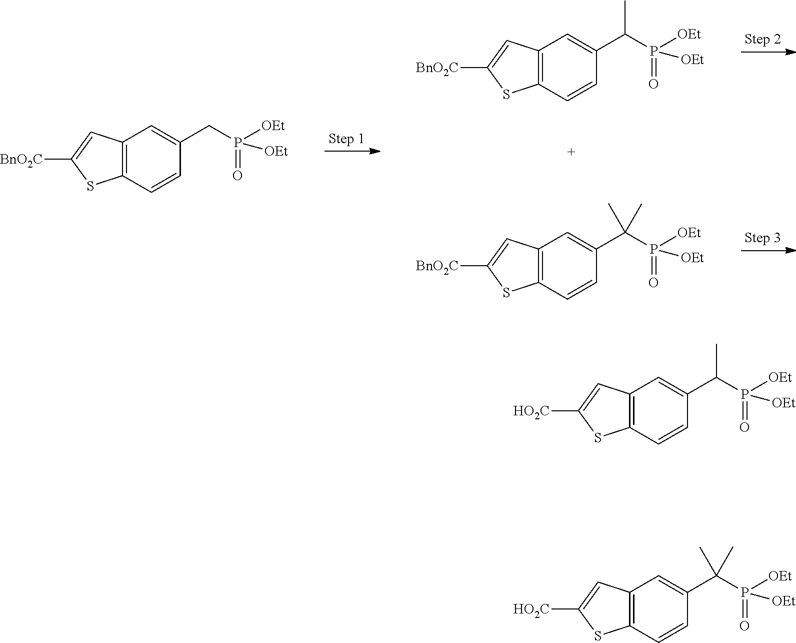

Step 1: Preparation of rac-benzyl 5-[1-(diethoxyphosphoryl)ethyl]-1-benzothiophene-2-carboxylate and benzyl 5-[2-(diethoxyphosphoryl)propan-2-yl]-1-benzothiophene-2-carboxylate

To a solution of benzyl 5-[(diethoxyphosphoryl)methyl]-1-benzothiophene-2-carboxylate (200 mg, 0.4779 mmol, 1 eq) and methyl iodide (88.5 μL, 1.43 mmol, 3 eq) in tetrahydrofuran (5 mL) at −78° C. was added a solution of sodium bis(trimethylsilylamide) (1 M in THF) (1.43 mL, 1.43 mmol, 3 eq) dropwise. The mixture was stirred at −78° C. for 2 h. The ice-bath was removed and the reaction was allowed to stir at room temperature for 1 h. The reaction mixture was quenched with saturated ammonium chloride aqueous solution (10 mL). The product was extracted with EtOAc (3×30 mL). The combined extracts were dried with anhydrous sodium sulfate, filtered and concentrated under reduced pressure. The crude residue was purified by reverse phase chromatography on a 50 g C₁₈ cartridge eluting with a gradient of 5-80% MeCN in water (with 0.1% formic acid) to give benzyl rac-5-[1-(diethoxyphosphoryl)ethyl]-1-benzothiophene-2-carboxylate (89.0 mg, 0.1993 mmol, 41.9%) as a clear oil and benzyl 5-[2-(diethoxyphosphoryl)propan-2-yl]-1-benzothiophene-2-carboxylate (22.0 mg, 0.5087 mmol, 10.6%) as a clear oil.

rac-Benzyl 5-[1-(diethoxyphosphoryl)ethyl]-1-benzothiophene-2-carboxylate LCMS: m/z=433.0 [M+H]⁺; ¹H NMR (400 MHz, DMSO-d₆) δ 8.08 (s, 1H), 7.88-7.85 (m, 1H), 7.83 (d, J=8.7 Hz, 1H), 7.52-7.47 (m, 3H), 7.45-7.36 (m, 3H), 5.42 (s, 2H), 4.12-4.02 (m, 2H), 4.00-3.91 (m, 1H), 3.89-3.78 (m, 1H), 3.38-3.25 (m, 1H), 1.66 (dt, J=18.3, 7.6 Hz, 3H), 1.30 (t, J=7.3 Hz, 3H), 1.16 (t, J=7.3 Hz, 3H).

Benzyl 5-[2-(diethoxyphosphoryl)propan-2-yl]-1-benzothiophene-2-carboxylate: LCMS: m/z=447.0 [M+H]⁺. ¹H NMR (400 MHz, DMSO-d₆) δ 8.01 (s, 1H), 7.93-7.90 (m, 1 J), 7.74 (d, J=8.6 Hz, 1H), 7.69-7.63 (m, 1H), 7.39 (d, J=8.6 Hz, 1H), 7.36-7.25 (m, 3H), 5.32 (s, 2H), 3.92-3.73 (m, 4H), 1.61 (d, J=16.8 Hz, 6H), 1.11 (t, J=7.2 Hz, 6H).

Step 2: Preparation of rac-5-[1-(diethoxyphosphoryl)ethyl]-1-benzothiophene-2-carboxylic acid

A mixture of 10% Pd/C (50% wet) (80 mg, 0.03758 mmol, 0.163 eq) and benzyl 5-[1-(diethoxyphosphoryl)ethyl]-1-benzothiophene-2-carboxylate (100 mg, 0.2312 mmol, 1 eq) in tetrahydrofuran (8 mL) was degassed with nitrogen for 5 min. Hydrogen was bubbled for 5 min. then the reaction was allowed to stir at room temperature under hydrogen (1 atm). The reaction was filtered over Celite and eluted with THF. The filtrate was concentrated under reduced pressure. The crude residue was purified by reverse phase chromatography on a 50 g C₁₈ cartridge eluting with a gradient of 5-80% MeCN in water (with 0.1% formic acid) to give rac-5-[1-(diethoxyphosphoryl)ethyl]-1-benzothiophene-2-carboxylic acid (51 mg, 0.1489 mmol, 65%) as a white solid. LCMS: m/z=343.0 [M+H]⁺.

Step 3: Preparation of 5-[2-(diethoxyphosphoryl)propan-2-yl]-1-benzothiophene-2-carboxylic acid

A mixture of 10% palladium on carbon (50% wet) (5 mg, 0.002349 mmol, eq) and 5-[2-(diethoxyphosphoryl)propan-2-yl]-1-benzothiophene-2-carboxylate (33 mg, 0.07390 mmol, 1 eq) in tetrahydrofuran (2 mL) was degassed with nitrogen for 5 min. Hydrogen was bubbled for 5 min. then the reaction was allowed to stir at room temperature under hydrogen (1 atm). The reaction was filtered on a syringe filter and eluted with MeOH. The filtrate was concentrated under reduced pressure. The crude residue was diluted in tetrahydrofuran (2 mL). 10% Palladium on carbon (50% wet) (20 mg, 0.009396 mmol, 0.13 eq) was added under nitrogen bubbling, then hydrogen was bubbled for 5 min. The reaction was stirred at room temperature overnight under hydrogen (1 atm). The reaction was filtered on a syringe filter and eluted with MeOH. The filtrate was concentrated under reduced pressure. The crude residue was diluted in tetrahydrofuran (2 mL) then 10% palladium on carbon (50% wet) (20 mg, 0.009396 mmol, 0.13 eq) was added under nitrogen bubbling, then hydrogen was bubbled for 5 min. The reaction was stirred at room temperature under hydrogen (1 atm) for 40 h. The reaction was filtered on a syringe filter and eluted with MeOH. The filtrate was concentrated under reduced pressure to give crude 5-[2-(diethoxyphosphoryl)propan-2-yl]-1-benzothiophene-2-carboxylic acid (26.3 mg, 0.0738 mmol, 99%) as a clear oil. LCMS: m/z=357.2 [M+H]⁺.

Synthesis of 5-((bis((((2-methoxyethoxy)carbonyl)oxy)methoxy)phosphoryl)difluoromethyl)benzo[b]thiophene-2-carboxylic acid

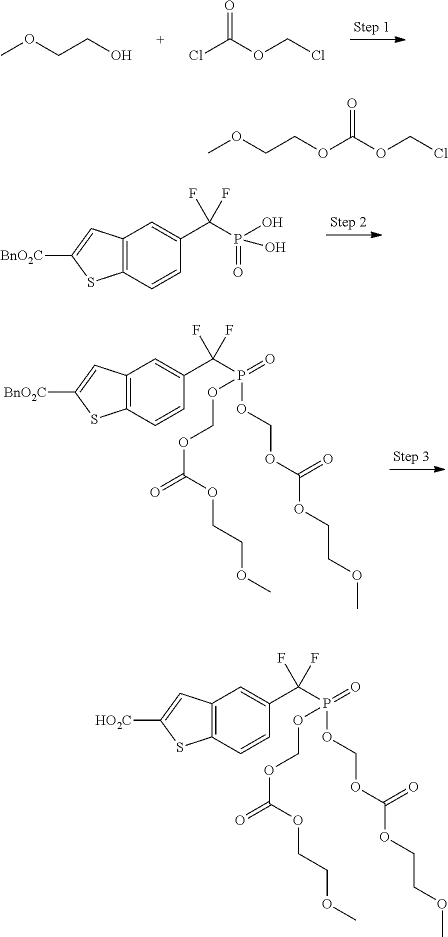

Step 1: Preparation of chloromethyl 2-methoxyethyl carbonate

A solution of 2-methoxyethan-1-ol (1 g, 13.1 mmol, 1 eq) and chloroacetyl chloride (1.68 g, 13.1 mmol, 1 eq) in diethyl ether (30 mL) was cooled down to 0° C. under nitrogen. Pyridine (1.04 mL, 13.1 mmol, 1.0 eq) was added dropwise and then the reaction was stirred for 15 min at 0° C. followed by 16 h at room temperature. The white suspension was filtered and rinsed with diethyl ether (30 mL). The filtrate was washed with 1 N HCl (20 mL) and water (2×20 mL) then dried over anhydrous sodium sulfate, filtered and concentrated under reduced pressure to give chloromethyl 2-methoxyethyl carbonate (1.63 g, 9.66 mmol, 74.0% yield) as a clear liquid.: ¹H NMR (400 MHz, CDCl₃) δ 5.76 (s, 2H), 4.42-4.37 (m, 2H), 3.68-3.64 (m, 2H), 3.42 (s, 3H).

Step 2: Preparation of benzyl 5-((bis((((2-methoxyethoxy)carbonyl)oxy)methoxy)phosphoryl)difluoromethyl)benzo[b]thiophene-2-carboxylate

Sodium hydroxide (59.9 mg, 1.50 mmol, 2 eq) in water (2 mL) was added dropwise to a stirred solution of ((2-((benzyloxy)carbonyl)benzo[b]thiophen-5-yl)difluoromethyl)phosphonic acid (300 mg, 0.7531 mmol, 1 eq) in water (15 mL). When the mixture reached pH˜8, silver nitrate (382 mg, 2.25 mmol, 3 eq) was added in one portion. After 2 h at room temperature, the grey suspension was cooled to 0° C. The precipitate was collected by filtration, washed with water, taken in MeCN, dried under reduced pressure and then dried under high vacuum for 2 h. The solid was suspended in dry toluene (10 mL), and chloromethyl 2-methoxyethyl carbonate (379 mg, 2.25 mmol, 3 eq) was added. The mixture was stirred for 18 h at room temperature then heated to 50° C. for 5 days. The mixture was adsorbed on silica gel and concentrated under reduced pressure. The crude residue was purified by flash-chromatography on a 24 g silica gel cartridge eluting with 0-90% EtOAc in heptane to give benzyl 5-((bis((((2-methoxyethoxy)carbonyl)oxy)methoxy)phosphoryl)difluoromethyl)benzo[b]thiophene-2-carboxylate (290 mg, 0.4377 mmol, 58.2% yield) as a clear oil. LCMS: m/z=663.2 [M+H]⁺. ¹H NMR (400 MHz, CDCl₃) δ 8.17 (s, 1H), 8.16-8.14 (m, 1H), 7.98 (d, J=8.4 Hz, 1H), 7.69 (d, J=8.4 Hz, 1H), 7.51-7.48 (m, 2H), 7.46-7.36 (m, 3H), 5.75 (dd, J=12.0, 5.5, Hz, 2H), 5.69 (dd, J=12.0, 5.5 Hz, 2H), 5.43 (s, 2H), 4.36-4.31 (m, 4H), 3.64-3.60 (m, 4H), 3.39 (s, 6H).

Step 3: Preparation of 5-((bis((((2-methoxyethoxy)carbonyl)oxy)methoxy)phosphoryl)difluoromethyl)benzo[b]thiophene-2-carboxylic acid

10% Palladium on carbon (50% wet) (145 mg, 0.1353 mmol, eq) was added to a mixture of benzyl 5-((bis((((2-methoxyethoxy)carbonyl)oxy)methoxy)phosphoryl)difluoromethyl)benzo[b]thiophene-2-carboxylate (145 mg, 0.2188 mmol, 1 eq) in anhydrous tetrahydrofuran (15 mL). Hydrogen was bubbled into the suspension for 2 min. and then the reaction mixture was stirred under hydrogen (1 atm) for 20 h. Nitrogen was bubbled and the reaction was filtered over Celite, rinsed with 2-MeTHF then concentrated under reduced pressure to give crude 5-((bis((((2-methoxyethoxy)carbonyl)oxy)methoxy)phosphoryl)difluoromethyl)benzo[b]thiophene-2-carboxylic acid (144 mg, 0.2188 mmol, 99% yield) as a grey solid. LCMS: m/z=573.2 [M+H]⁺.

The following linkers in Table 14 were prepared according to the procedure described for the synthesis of 5-((bis((((2-methoxyethoxy)carbonyl)oxy)methoxy)phosphoryl)difluoromethyl)benzo[b]thiophene-2-carboxylic acid utilizing the appropriate starting materials.

TABLE 14

| Name | Structure | LCMS |
|---|---|---|
| 5-((bis((((3- methoxypropoxy)carbonyl)oxy)methoxy) phosphoryl)difluoromethyl)benzo[b] thiophene- 2-carboxylic acid | 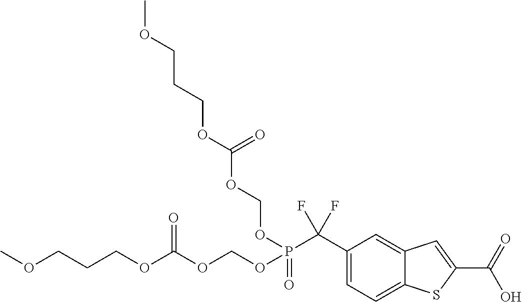 [SMILES: COCCCOC(=O)OCOP(=O)(OCOC(=O)OCCCOC)C(F)(F)c1ccc2sc(C(=O)O)cc2c1] | 601.4 [M + H]⁺ |

Synthesis of 5-((bis(4-ethoxy-4-oxobutoxy)phosphoryl)difluoromethyl)benzo[b]thiophene-2-carboxylic acid

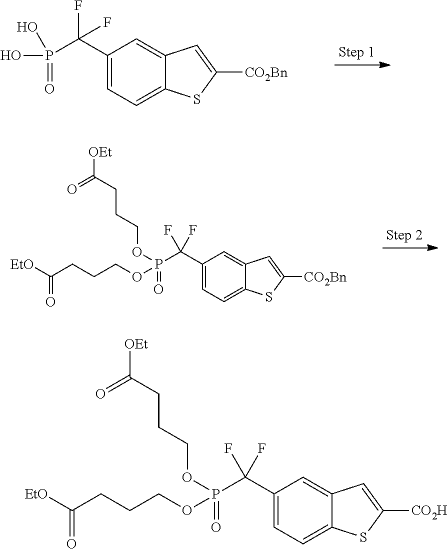 [SMILES: O=C(OCc1ccccc1)c1cc2cc(C(F)(F)P(=O)(O)O)ccc2s1 ; CCOC(=O)CCCOP(=O)(OCCCC(=O)OCC)C(F)(F)c1ccc2sc(C(=O)OCc3ccccc3)cc2c1 ; CCOC(=O)CCCOP(=O)(OCCCC(=O)OCC)C(F)(F)c1ccc2sc(C(=O)O)cc2c1]

Step 1: Preparation of diethyl 4,4′-((((2-((benzyloxy)carbonyl)benzo[b]thiophen-5-yl)difluoromethyl)phosphoryl)bis(oxy))dibutyrate

Sodium hydroxide (59.9 mg, 1.50 mmol) in H₂O (2 mL) was added dropwise to a stirred solution of ((2-((benzyloxy)carbonyl)benzo[b]thiophen-5-yl)difluoromethyl)phosphonic acid (300 mg, 0.7531 mmol, 1 eq)) in H₂O (15 mL). When the mixture became pH˜8, silver nitrate (382 mg, 2.25 mmol) was added. After 2 h at room temperature, the grey suspension was cooled to 0° C. The precipitate was collected by filtration, washed with water, taken in MeCN, concentrated under reduced pressure and then dried under high vacuum. The solid was suspended in dry toluene (10 mL) and ethyl 4-bromobutanoate (438 mg, 2.25 mmol, 3 eq) was added. The mixture was stirred for 18 h at 50° C. The mixture was adsorbed on silica gel and concentrated under reduced pressure. The crude residue was purified by flash-chromatography on a 24 g silica gel cartridge eluting with 0-90% EtOAc in heptane to give diethyl 4,4′-((((2-((benzyloxy)carbonyl)benzo[b]thiophen-5-yl)difluoromethyl)phosphoryl)bis(oxy))dibutyrate (320 mg, 0.5106 mmol, 67.9% yield) as a clear oil. ¹H NMR (400 MHz, CDCl₃) δ 8.18-8.13 (m, 2H), 7.97 (d, J=8.6 Hz, 1H), 7.70 (d, J=8.5 Hz, 1H), 7.53-7.35 (m, 5H). 5.43 (s, 2H), 4.28-4.09 (m, 8H), 2.44-2.36 (m, 4H), 2.04-1.92 (m, 4H), 1.32-1.22 (m, 6H).

Step 2: Preparation of 5-((bis(4-ethoxy-4-oxobutoxy)phosphoryl)difluoromethyl)benzo[b]thiophene-2-carboxylic acid

10% Palladium on carbon (50% wet) (100 mg, 0.04698 mmol, 0.3 eq) was added to a mixture of diethyl 4,4′-((((2-((benzyloxy)carbonyl)benzo[b]thiophen-5-yl)difluoromethyl)phosphoryl)bis(oxy))dibutyrate (100 mg, 0.1595 mmol, 1 eq) in anhydrous tetrahydrofuran (10 mL). Hydrogen was bubbled into the suspension for 2 min. and then the reaction mixture was stirred under 1 atm. of hydrogen for 16 h. Nitrogen was bubbled in the reaction mixture and it was then filtered over Celite (rinsing with 2-MeTHF) and concentrated under reduced pressure to give 5-((bis(4-ethoxy-4-oxobutoxy)phosphoryl)difluoromethyl)benzo[b]thiophene-2-carboxylic acid (85.3 mg, 0.1595 mmol, 100% yield) as a clear oil. LCMS: m/z=559.2 [M+Na]⁺.

Synthesis of 5-(cyano(ethoxy(hydroxy)phosphoryl)methyl)benzo[b]thiophene-2-carboxylic acid

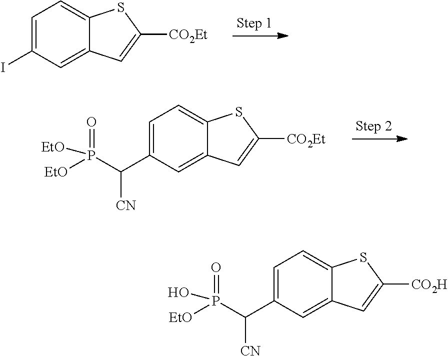 [SMILES: CCOC(=O)c1cc2cc(I)ccc2s1 ; CCOC(=O)c1cc2cc(C(C#N)P(=O)(OCC)OCC)ccc2s1 ; CCOP(=O)(O)C(C#N)c1ccc2sc(C(=O)O)cc2c1]

Step 1: Preparation of ethyl 5-(cyano(diethoxyphosphoryl)methyl)benzo[b]thiophene-2-carboxylate

To a solution of diethyl (cyanomethyl)phosphonate (127 mg, 722 μmol, 1.2 eq) in anhydrous 1,2-dimethoxyethane (5 mL) under nitrogen was added sodium hydride (60% in mineral oil) (50.3 mg, 1.26 mmol, 2.1 eq). The reaction was stirred at r.t. for 10 min. and then tetrakis(triphenylphosphine)palladium (34.7 mg, 30.1 μmol, 0.05 eq) and ethyl 5-iodo-1-benzothiophene-2-carboxylate (200 mg, 602 μmol, 1 eq) were added. The reaction mixture was stirred at 85° C. for 16 h. The reaction mixture was filtered over Celite (eluting with DCM) and the filtrate was concentrated under reduced pressure. The crude residue was purified by reverse phase chromatography on a 50 g C₁₈ cartridge eluting with 5-80% MeCN in water. The combined fractions were concentrated to give ethyl 5-(cyano(diethoxyphosphoryl)methyl)benzo[b]thiophene-2-carboxylate (50.0 mg, 131 μmol, 21.8% yield) as an orange solid. LCMS: m/z=382.2 [M+H]⁺; ¹H NMR (400 MHz, DMSO-d₆) δ 8.33 (s, 1H), 8.18-8.06 (m, 2H), 7.54 (d, J=8.6 Hz, 1H), 5.67-5.56 (m, 1H), 4.37 (q, J=7.1 Hz, 2H), 4.11-3.97 (m, 4H), 1.34 (t, J=7.1 Hz, 3H), 1.20 (dt, J=9.8, 7.1 Hz, 6H).

Step 2: Preparation of 5-(cyano(ethoxy(hydroxy)phosphoryl)methyl)benzo[b]thiophene-2-carboxylic acid

To a solution of ethyl 5-(cyano(diethoxyphosphoryl)methyl)benzo[b]thiophene-2-carboxylate (80 mg, 209 μmol, 1 eq) in acetonitrile (4 mL) was added hydrochloric acid (6 mL, 3 N). The reaction mixture was stirred at 70° C. for 7 h. More hydrochloric acid (1.5 mL, 3 N) was added and the reaction mixture was stirred at 70 C for additional 22 h. Hydrochloric acid (1.5 mL, 3 N) was added and the reaction mixture was stirred at 80° C. for additional 20 h. The reaction mixture was concentrated under reduced pressure. The crude was directly purified by reverse phase chromatography using a 50 g C₁₈ cartridge eluting with a gradient of MeCN in water (5% for 3 CV then 5 to 100% in 18 CV) to give 5-(cyano(ethoxy(hydroxy)phosphoryl)methyl)benzo[b]thiophene-2-carboxylic acid (52.0 mg, 159 μmol, 76.5% yield) as a white solid. ¹H NMR (400 MHz, DMSO-d₆) δ 8.19 (s, 1H), 8.12-7.98 (m, 2H), 7.52 (d, J=8.6 Hz, 1H), 5.23-5.12 (m, 1H), 3.97 (quin, J=7.3 Hz, 2H), 1.22-1.13 (m, 3H).

Synthesis of rac-5-[1-(diethoxyphosphoryl)-1-fluoroethyl]-1-benzothiophene-2-carboxylic acid

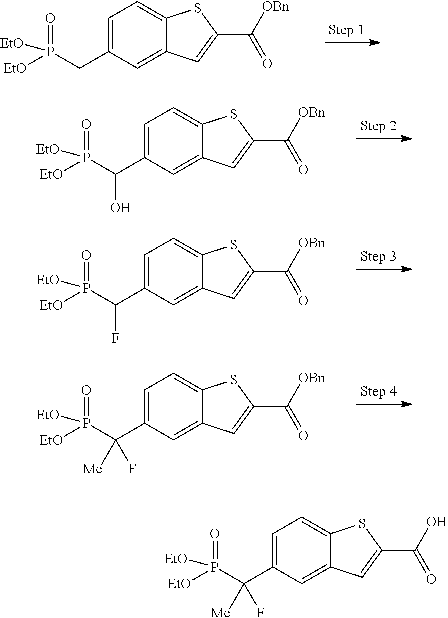 [SMILES: CCOP(=O)(Cc1ccc2sc(C(=O)OCc3ccccc3)cc2c1)OCC ; CCOP(=O)(OCC)C(O)c1ccc2sc(C(=O)OCc3ccccc3)cc2c1 ; CCOP(=O)(OCC)C(F)c1ccc2sc(C(=O)OCc3ccccc3)cc2c1 ; CCOP(=O)(OCC)C(C)(F)c1ccc2sc(C(=O)OCc3ccccc3)cc2c1 ; CCOP(=O)(OCC)C(C)(F)c1ccc2sc(C(=O)O)cc2c1]

Step 1: Preparation of rac-benzyl 5-[(diethoxyphosphoryl)(hydroxy)methyl]-1-benzothiophene-2-carboxylate

A solution of benzyl 5-[(diethoxyphosphoryl)methyl]-1-benzothiophene-2-carboxylate (2.4 g, 5.73 mmol, 1 eq) in tetrahydrofuran (75 mL) was cooled down to −78° C. then 2-(benzenesulfonyl)-3-phenyloxaziridine (2.97 g, 11.4 mmol, 2 eq) was added followed by the addition of a solution of sodium bis(trimethylsilylamide) (1.0 M in THF) (11.4 mL, 11.4 mmol, 2 eq) dropwise (internal T°=−75° C. to −69° C.). A deep purple solution was observed upon addition of base and changed rapidly to orange. The mixture was stirred for 10 min. at −78° C. The reaction mixture was quenched with saturated NH₄Cl aqueous solution (50 mL) at −78° C. then the dry ice bath was removed. EtOAc (75 mL) and water (25 mL) were added, and the mixture was stirred for 30 min. (until internal temperature reached 15° C.). The phases were separated and then the aqueous layer was back extracted with EtOAc (125 mL). The combined organic extracts were dried with anhydrous sodium sulfate, filtered, adsorbed on silica gel and concentrated under reduced pressure. The crude material was combined with another batch (6.42 mmol, 12.15 mmol in total) and was purified by flash-chromatography on a 330 g silica gel cartridge eluting with 20-100% EtOAc in heptane to give rac-benzyl 5-[(diethoxyphosphoryl)(hydroxy)methyl]-1-benzothiophene-2-carboxylate (3.69 g, 8.49 mmol, 70% yield) as a white sticky solid. LCMS: m/z=869.4 (2M+H)⁺; ¹H NMR (400 MHz, CDCl₃) δ 8.10 (s, 1H), 8.04-8.00 (m, 1H), 7.88 (d, J=8.6 Hz, 1H), 7.61 (d, J=8.6 Hz, 1H), 7.51-7.47 (m, 2H), 7.46-7.35 (m, 3H), 5.42 (s, 2H), 5.17 (dd, J=10.4, 4.5 Hz, 1H), 4.18-3.95 (m, 4H), 3.10-2.99 (m, 1H), 1.33-1.20 (m, 6H).

Step 2: Preparation of rac-benzyl 5-[(diethoxyphosphoryl)(fluoro)methyl]-1-benzothiophene-2-carboxylate

To a solution of rac-benzyl 5-[(diethoxyphosphoryl)(hydroxy)methyl]-1-benzothiophene-2-carboxylate (1.56 g, 3.59 mmol, 1 eq) in methylene chloride (30 mL) at −78° C. under nitrogen was added (diethylamino)sulfur trifluoride (568 L, 4.30 mmol, 1.2 eq). The reaction was stirred for 15 min. at −78° C. The reaction was quenched with the addition of a saturated sodium bicarbonate aqueous solution (50 mL) and the product was extracted with DCM (3×50 mL). The combined organic layers were dried over anhydrous sodium sulfate, filtered and concentrated under reduced pressure. The crude residue was purified by reverse phase chromatography on a 275 g C₁₈ cartridge eluting with 5-80% MeCN in water to give rac-benzyl 5-[(diethoxyphosphoryl)(fluoro)methyl]-1-benzothiophene-2-carboxylate (650 mg, 1.48 mmol, 41.6% yield) as a thick clear oil. LCMS: m/z=873.2 (2M+H)⁺; ¹H NMR (400 MHz, CDCl₃) δ 8.12 (s, 1H), 8.02-7.99 (m, 1H), 7.92 (d, J=8.7 Hz, 1H), 7.61 (d, J=8.7 Hz, 1H), 7.51-7.51 (m, 2H), 7.46-7.36 (m, 3H), 5.82 (dd, J=44.4, 7.5 Hz, 1H), 5.42 (s, 2H), 4.21-4.02 (m, 4H), 1.34-1.26 (m, 6H).

Step 3: Preparation of rac-benzyl 5-[1-(diethoxyphosphoryl)-1-fluoroethyl]-1-benzothiophene-2-carboxylate

To a solution of benzyl 5-[(diethoxyphosphoryl)(fluoro)methyl]-1-benzothiophene-2-carboxylate (100 mg, 0.2291 mmol, 1 eq) and methyl iodide (42.7 μL, 687 μmol, 3 eq) in tetrahydrofuran (4 mL) at −78° C. was added a solution of sodium bis(trimethylsilylamide) (1 M in THF) (458 μL, 458 μmol, 2 eq) dropwise. The mixture was stirred at −78° C. for 5 min. The reaction was quenched with 1 M HCl (10 mL), warmed up to room temperature and extracted with DCM (2×25 mL). The combined organic extracts were dried over anhydrous sodium sulfate, filtered and concentrated under reduced pressure. The crude material was purified by reverse phase chromatography on 50 g C₁₈ cartridge eluting with 5-80% MeCN in water (containing 0.1% formic acid). The fractions were combined, concentrated under reduced pressure then freeze dried to give rac-benzyl 5-[1-(diethoxyphosphoryl)-1-fluoroethyl]-1-benzothiophene-2-carboxylate (67.0 mg, 0.1487 mmol, 65.0% yield) as a yellowish oil. LCMS: m/z=451.2 [M+H]⁺; ¹H NMR (400 MHz, CDCl₃) δ 8.13 (s, 1H), 8.05-8.02 (m, 1H), 7.90 (d, J=8.7 Hz, 1H), 7.65 (d, J=8.7 Hz, 1H), 7.52-7.47 (m, 2H), 7.46-7.36 (m, 3H), 5.42 (s, 2H), 4.27-4.16 (m, 2H), 4.05-3.94 (m, 1H), 3.90-3.79 (m, 1H), 2.03 (dd, J=25.0, 14.0 Hz, 3H), 1.37 (t, J=7.1 Hz, 3H), 1.17 (t, J=7.1 Hz, 3H).

Step 4: Preparation of rac-5-[1-(diethoxyphosphoryl)-1-fluoroethyl]-1-benzothiophene-2-carboxylic acid

A mixture of 10% palladium on carbon (50% wet) (99 mg, 0.04651 mmol, 0.21 eq) and benzyl 5-[1-(diethoxyphosphoryl)-1-fluoroethyl]-1-benzothiophene-2-carboxylatebenzyl 5-[1-(diethoxyphosphoryl)-1-fluoroethyl]-1-benzothiophene-2-carboxylate (99 mg, 0.2197 mmol, 1 eq) in tetrahydrofuran (6 mL) was degassed with nitrogen for 5 min. Hydrogen was bubbled for 5 min. and then the reaction was allowed to stir at room temperature under hydrogen (1 atm) for 20 h. The reaction was filtered over a syringe filter and rinsed with THF. The filtrate was concentrated under reduced pressure to give crude rac-5-[1-(diethoxyphosphoryl)-1-fluoroethyl]-1-benzothiophene-2-carboxylic acid (78.9 mg, 0.2197 mmol, 100% yield) as a clear oil. LCMS: m/z=361.2 [M+H]⁺.

Synthesis of rac-7-[(diethoxyphosphoryl)(fluoro)methyl]naphthalene-2-carboxylic acid

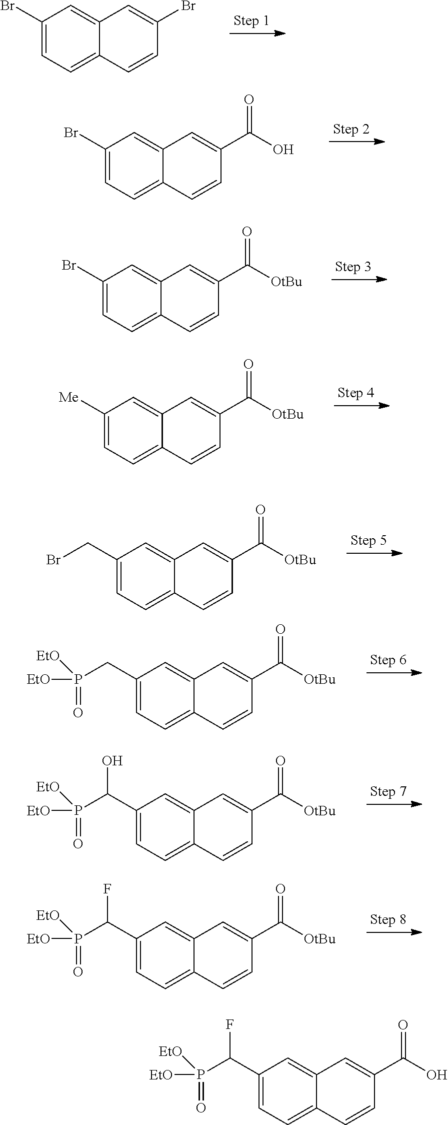

Step 1: Preparation of 7-bromonaphthalene-2-carboxylic acid

A solution of 2,7-dibromonaphthalene (2 g, 6.99 mmol, 1 eq) in tetrahydrofuran (24 mL) was cooled down to −78° C. under nitrogen. A solution of n-butyllithium (1.6 M in hexanes) (4.58 mL, 7.33 mmol, 1.05 eq) was added dropwise. The reaction was stirred for 15 min. at −78° C. then CO₂ was bubbled into the reaction mixture. The ice-bath was removed, and the reaction was stirred for 1 h under CO₂ bubbling. The reaction was quenched with the addition of 1 N HCl (50 mL, pH=2) and then the product was extracted with EtOAc (3×75 mL). The combined organic layers were washed with water (50 mL), brine (50 mL) then dried over anhydrous sodium sulfate, filtered and concentrated under reduced pressure. The crude residue was triturated in heptane and the resulting solid was filtered and dried to give 7-bromonaphthalene-2-carboxylic acid (1.27 g, 5.05 mmol, 72.5% yield) as a white solid. ¹H NMR (400 MHz, CDCl₃) δ 13.21 (s, 1H), 8.61 (s, 1H), 8.44 (s, 1H), 8.08-7.96 (m, 3H), 7.81-7.76 (m, 1H).

Step 2: Preparation of tert-butyl 7-bromonaphthalene-2-carboxylate

To a stirring suspension of 7-bromonaphthalene-2-carboxylic acid (1.00 g, 3.98 mmol, 1 eq) in toluene (9 mL) at 90° C. was added N,N-dimethylformamide dibutyl acetal (3.80 mL, 15.9 mmol, 4 eq) over 15 min. The reaction was stirred for 2 h at 90° C. then an additional amount of N, N-dimethylformamide dibutyl acetal (0.9 mL, 3.38 mmol, 1 eq) was added dropwise and the reaction was stirred for 30 min. at 90° C. The reaction was cooled down to room temperature and stirred overnight. The reaction was adsorbed on silica gel and concentrated under reduced pressure. The crude residue was purified by flash-chromatography on 40 g silica gel cartridge eluting with a gradient of 0-10% EtOAc in heptane to give tert-butyl 7-bromonaphthalene-2-carboxylate (1.11 g, 3.61 mmol, 90.9% yield) as a white solid. ¹H NMR (400 MHz, CDCl₃) δ 8.46 (s, 1H), 8.14 (s, 1H), 8.06 (dd, J=8.6, 1.6 Hz, 1H), 7.86 (d, J=8.6 Hz, 1H), 7.77 (d, J=8.6, Hz, 1H), 7.66 (dd, J=8.6, 1.6 Hz, 1H), 1.67 (s, 9H).

Step 3: Preparation of tert-butyl 7-methylnaphthalene-2-carboxylate

A mixture of tert-butyl 7-bromonaphthalene-2-carboxylate (1.11 g, 3.61 mmol, 1 eq), trimethylboroxine (906 mg, 7.22 mmol, 2 eq), tetrakis(triphenylphosphine)palladium (834 mg, 722 μmol, 0.2 eq) and potassium carbonate (1.99 g, 14.4 mmol, 4 eq) in 1,4-dioxane (20 mL) was degassed for 5 min. and then sealed and heated to 110° C. for 20 h. The reaction was diluted with EtOAc (20 mL) and adsorbed on silica gel. The crude residue was purified by flash-chromatography eluting with 0-10% EtOAc in heptane to give tert-butyl 7-methylnaphthalene-2-carboxylate (808 mg, 3.33 mmol, 92.4% yield) as a white crystalline solid. ¹H NMR (400 MHz, CDCl₃) δ 8.45 (s, 1H), 7.95 (d, J=8.7, Hz, 1H), 7.80 (d, J=8.5, Hz, 1H), 7.77 (d, J=8.5 Hz, 1H), 7.71 (s, 1H), 7.40 (d, J=8.7 Hz, 1H), 2.53 (s, 3H), 1.65 (s, 9H).

Step 4: Preparation of tert-butyl 7-(bromomethyl)naphthalene-2-carboxylate

To a solution of tert-butyl 7-methylnaphthalene-2-carboxylate (808 mg, 3.33 mmol) in anhydrous carbon tetrachloride (25 mL) under nitrogen atmosphere was added N-bromosuccinimide (621 mg, 3.49 mmol) and benzoyl peroxide (32.2 mg, 133 μmol). The reaction mixture was heated at reflux and stirred at this temperature for 20 h. The precipitate was filtered off, washed with carbon tetrachloride (10 mL) and then the filtrate was adsorbed on silica gel and concentrated under reduced pressure. The crude material was purified by flash-chromatography on an 80 g silica gel cartridge eluting with 0-10% EtOAc in heptane to give tert-butyl 7-(bromomethyl)naphthalene-2-carboxylate (720 mg, 2.24 mmol, 67.9% yield) as a white solid which contained residual starting material (˜25%). ¹H NMR (400 MHz, CDCl₃) δ 8.50 (s, 1H), 8.04 (dd, J=8.6, 1.6 Hz, 1H), 7.93 (s, 1H), 7.85 (t, J=8.8 Hz, 2H), 7.60 (dd, J=8.6, 1.6 Hz, 1H), 4.67 (s, 2H), 1.64 (s, 9H).

Step 5: Preparation of tert-butyl 7-[(diethoxyphosphoryl)methyl]naphthalene-2-carboxylate

tert-Butyl 7-(bromomethyl)naphthalene-2-carboxylate (720 mg, 2.24 mmol) was suspended in triethyl phosphite (2 mL, 11.6 mmol) and the reaction mixture was heated for 1.5 h at reflux (became a solution once at 110° C.). The reaction was cooled down to room temperature and directly purified through reverse phase chromatography on a 50 g Cis cartridge using a gradient of 5-80% MeCN in water to tert-butyl 7-[(diethoxyphosphoryl)methyl]naphthalene-2-carboxylate (580 mg, 1.53 mmol, 68.4% yield) as a thick yellowish oil. LCMS: m/z=379.3 [M+H]⁺; ¹H NMR (400 MHz, CDCl₃) δ 8.52 (s, 1H), 8.08-8.00 (m, 1H), 7.89-7.83 (m, 3H), 7.58-7.54 (m, 1H), 4.11-3.97 (m, 4H), 3.36 (d, J=21.6 Hz, 2H), 1.67 (s, 9H), 1.26 (t, J=7.0 Hz, 6H).

Step 6: Preparation of rac-tert-butyl 7-[(diethoxyphosphoryl)(hydroxy)methyl]naphthalene-2-carboxylate

To a solution of tert-butyl 7-[(diethoxyphosphoryl)methyl]naphthalene-2-carboxylate (200 mg, 0.5285 mmol, 1 eq) in tetrahydrofuran (5 mL) at −78° C. was added a solution of sodium bis(trimethylsilylamide) (1 M in THF) (792 μL, 792 μmol, 1.5 eq) dropwise. The mixture was stirred for 2 min. and then 2-(benzenesulfonyl)-3-phenyloxaziridine (274 mg, 1.05 mmol, 2.0 eq) was added portion wise. The dark red solution was stirred at −78° C. for 20 min. The reaction mixture was quenched with saturated ammonium chloride aqueous solution (50 mL) at −78° C. and then the ice-bath was removed. EtOAc (25 mL) and water (25 mL) were added and the mixture was stirred for 30 min. The phases were separated and then the aqueous layer was back extracted with EtOAc (2×25 mL). The combined organic extracts were dried with anhydrous sodium sulfate, filtered and concentrated under reduced pressure. The crude material was purified by flash-chromatography on a 24 g silica gel cartridge eluting with 20-100% EtOAc in heptane to give rac-tert-butyl 7-[(diethoxyphosphoryl)(hydroxy)methyl]naphthalene-2-carboxylate (140 mg, 0.3549 mmol, 67.3% yield) as a white sticky solid. LCMS: m/z=394.6 [M+H]⁺; ¹H NMR (400 MHz, CDCl₃) δ 8.57 (s, 1H), 8.08 (s, 1H), 8.05 (d, J=8.6, Hz, 1H), 7.90 (d, J=8.3 Hz, 1H), 7.87 (d, J=8.3 Hz, 1H), 7.72 (d, J=8.3 Hz, 1H), 5.24 (dd, J=11.1, 4.7 Hz, 1H), 4.18-3.99 (m, 4H), 3.31 (dd, J=11.1, 4.7 Hz, 1H), 1.66 (s, 9H), 1.32-1.22 (m, 6H).

Step 7: Preparation of rac-tert-butyl 7-[(diethoxyphosphoryl)(fluoro)methyl]naphthalene-2-carboxylate

To a solution of tert-butyl 7-[(diethoxyphosphoryl)(hydroxy)methyl]naphthalene-2-carboxylate (140 mg, 0.3549 mmol, 1 eq) in methylene chloride (5 mL) at −78° C. under nitrogen was added a solution of (diethylamino)sulfur trifluoride (56.1 μL, 425 μmol, 1.2 eq) dropwise. The reaction was stirred for 20 min at −78° C. The reaction was quenched with the addition of saturated sodium bicarbonate aqueous solution (50 mL) and extracted with DCM (3×50 mL). The combined organic layers were dried over anhydrous sodium sulfate, filtered and concentrated under reduced pressure. The crude residue was purified by reverse phase chromatography on a 50 g C₁₈ cartridge eluting with a gradient of 5-80% MeCN in water to give rac-tert-butyl 7-[(diethoxyphosphoryl) (fluoro)methyl]naphthalene-2-carboxylate (80.0 mg, 0.2018 mmol, 57.1% yield) as a clear oil. LCMS: m/z=397.4 [M+H]⁺; ¹H NMR (400 MHz, CDCl₃) δ 8.59 (s, 1H), 8.11-8.04 (m, 2H), 7.94 (d, J=8.3, Hz, 1H), 7.90 (d, J=8.3 Hz, 1H), 7.72 (d, J=8.6, 1H), 5.90 (dd, J=44.6, 8.1 Hz, 1H), 4.24-4.02 (m, 4H), 1.35-1.25 (m, 6H).

Step 8: Preparation of rac-7-[(diethoxyphosphoryl)(fluoro)methyl]naphthalene-2-carboxylic acid

To a solution of rac-tert-butyl 7-[(diethoxyphosphoryl)(fluoro)methyl]naphthalene-2-carboxylate (80 mg, 0.2018 mmol) in methylene chloride (4 mL) was added trifluoroacetic acid (1 mL). The resulting yellow solution was stirred for 2.5 h at room temperature. The reaction was concentrated under reduced pressure to give crude rac-7-[(diethoxyphosphoryl)(fluoro)methyl]naphthalene-2-carboxylic acid (68.3 mg, 0.2018 mmol) as a clear oil. LCMS: m/z=341.2 [M+H]⁺.

Synthesis of 5-{[bis({[(2S)-2-(methoxycarbonyl)pyrrolidine-1-carbonyloxy]methoxy})phosphoryl]difluoromethyl}-1-benzothiophene-2-carboxylic acid

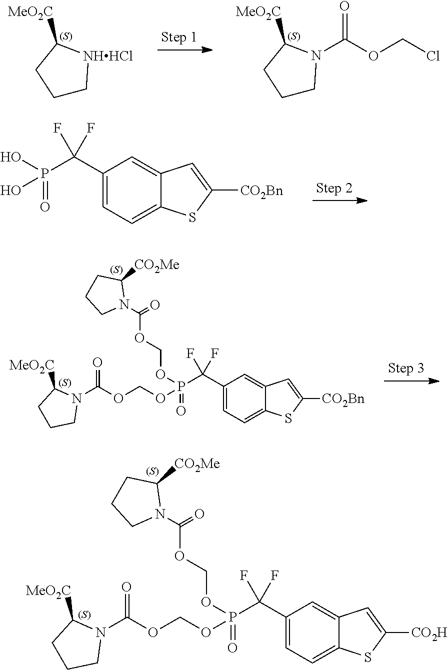

Step 1: Preparation of 1-chloromethyl 2-methyl (2S)-pyrrolidine-1,2-dicarboxylate

Chloroacetyl chloride (777 mg, 6.03 mmol) was added to a solution of methyl (2S)-pyrrolidine-2-carboxylate hydrochloride (1.00 g, 6.03 mmol) and N,N-diisopropylethylamine (818 mg, 6.33 mmol, 1.05 eq) in dichloromethane (15 mL) at 0° C. under nitrogen atmosphere. After stirring for 0.5 h, the reaction mixture was washed successively with 1 N aqueous hydrochloric acid (50 mL), water (50 mL) and brine. The organic layer was dried over anhydrous sodium sulfate, filtered and concentrated under reduced pressure. The product was purified by flash-chromatography using a 25 g silica gel cartridge eluting with a gradient of EtOAc in heptane (0 to 50%). The combined fractions were concentrated under reduced pressure to afford 1-chloromethyl 2-methyl (2S)-pyrrolidine-1,2-dicarboxylate (701 mg, 3.16 mmol, 52.7% yield). ¹H NMR (400 MHz, CDCl₃) δ 5.88 (d, J=6.0 Hz, 0.5H), 5.76-5.70 (m, 1H), 5.65 (d, J=6.2 Hz, 0.5H), 4.39 (ddd, J=18.8, 8.5, 3.6 Hz, 1H), 3.74 (d, J=3.4 Hz, 3H), 3.68-3.45 (m, 2H), 2.32-2.16 (m, 1H), 2.10-1.87 (m, 3H).

Step 2: Preparation of 1-{[({2-[(benzyloxy)carbonyl]-1-benzothiophen-5-yl}difluoromethyl)({[(2S)-2-(methoxycarbonyl)pyrrolidine-1-carbonyloxy]methoxy})phosphoryl]oxy}methyl 2-methyl (2S)-pyrrolidine-1,2-dicarboxylate

Sodium hydroxide (59.9 mg, 1.50 mmol) in water (2 mL) was added dropwise to a stirred solution of ({2-[(benzyloxy)carbonyl]-1-benzothiophen-5-yl}difluoromethyl)phosphonic acid (300 mg, 753 μmol, 1 eq) in water (15 mL) then silver nitrate (382 mg, 2.25 mmol) was added. After 2 h at r.t., the suspension was cooled to 0° C. The precipitate was collected by filtration, washed with water, co-evaporated with toluene (2×) and dried under high vacuum. The powder was suspended in dry toluene (10 mL) and 1-chloromethyl 2-methyl (2S)-pyrrolidine-1,2-dicarboxylate (582 mg, 2.63 mmol, 3.5 eq) was added. The mixture was stirred for 18 h at room temperature. The crude was directly purified by normal phase chromatography using a 12 g silica gel cartridge eluting with a gradient of EtOAc in heptane (0 to 100% in 18 CV) to afford 1-{[({2-[(benzyloxy)carbonyl]-1-benzothiophen-5-yl}difluoromethyl)({[(2S)-2-(methoxycarbonyl)pyrrolidine-1-carbonyloxy]methoxy})phosphoryl]oxy}methyl 2-methyl (2S)-pyrrolidine-1,2-dicarboxylate (90.0 mg, 117 μmol, 15.5% yield) as a white solid. ¹H NMR (400 MHz, CDCl₃) δ 8.20-8.12 (m, 2H), 7.97-7.91 (m, 1H), 7.69-7.64 (m, 1H), 7.48-7.33 (m, 5H), 5.79-5.66 (m, 2H), 5.66-5.52 (m, 2H), 5.41-5.38 (m, 2H), 4.40-4.31 (m, 2H), 3.75-3.65 (m, 6H), 3.63-3.40 (m, 4H), 2.25-2.11 (m, 2H), 2.04-1.81 (m, 6H).

Step 3: Preparation of 5-{[bis({[(2S)-2-(methoxycarbonyl)pyrrolidine-1-carbonyloxy]methoxy})phosphoryl]difluoromethyl}-1-benzothiophene-2-carboxylic acid

Palladium on carbon (10% loading, 50% wet support) (62.1 mg, 58.5 μmol, 0.5 eq) was added to a mixture of 1-{[({2-[(benzyloxy)carbonyl]-1-benzothiophen-5-yl}difluoromethyl) ({[(2S)-2-(methoxycarbonyl)pyrrolidine-1-carbonyloxy]methoxy})phosphoryl]oxy}methyl 2-methyl (2S)-pyrrolidine-1,2-dicarboxylate (90 mg, 117 μmol, 1 eq) in anhydrous tetrahydrofuran (10 mL). Hydrogen was bubbled into the suspension for 2 min. and then the reaction mixture was stirred under 1 atm (balloon) of hydrogen for 16 h. The reaction mixture was filtered on Celite and rinsed with Me-THF. The filtrate was concentrated in vacuo to give crude 5-{[bis({[(2S)-2-(methoxycarbonyl)pyrrolidine-1-carbonyloxy]methoxy})phosphoryl]difluoromethyl}-1-benzothiophene-2-carboxylic acid (95.0 mg, 140 μmol, 119% yield) as a colorless oil. ¹H NMR (400 MHz, CDCl₃) δ 8.18-8.13 (m, 1H), 8.13-8.07 (m, 1H), 7.97-7.92 (m, 1H), 7.69-7.63 (m, 1H), 5.82-5.58 (m, 4H), 4.43-4.34 (m, 2H), 3.77-3.67 (m, 6H), 3.64-3.42 (m, 4H), 2.27-2.15 (m, 2H), 2.09-1.82 (m, 6H).

The following linkers in Table 15 were prepared according to the procedure described for the synthesis of 5-{[bis({[(2S)-2-(methoxycarbonyl)pyrrolidine-1-carbonyloxy]methoxy})phosphoryl]difluoromethyl}-1-benzothiophene-2-carboxylic acid utilizing the appropriate starting materials.

TABLE 15

| Name | Structure | LCMS |
|---|---|---|
| 5-((bis(((((S)-1-ethoxy- 1-oxopropan-2- yl)carbamoyl)oxy) methoxy)phosphoryl) methyl)benzo[b] thiophene-2- carboxylic acid | 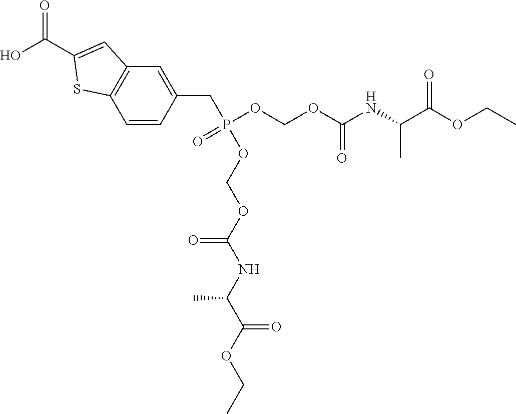 [SMILES: CCOC(=O)[C@H](C)NC(=O)OCOP(=O)(Cc1ccc2sc(C(=O)O)cc2c1)OCOC(=O)N[C@@H](C)C(=O)OCC] | 619.2 [M + H]⁺ |

Synthesis of 5-(((benzylamino)(((S)-1-isopropoxy-1-oxopropan-2-yl)amino)phosphoryl)methyl)benzo[b]thiophene-2-carboxylic acid

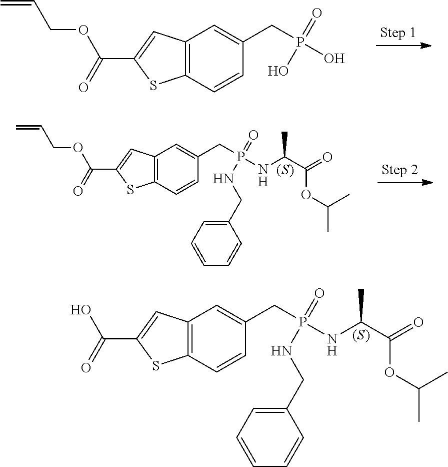

Step 1: Preparation of allyl 5-(((benzylamino)(((S)-1-isopropoxy-1-oxopropan-2-yl)amino)phosphoryl)methyl)benzo[b]thiophene-2-carboxylate

Oxalyl chloride (814.4 mg, 6.4 mmol, 10 eq.) was added dropwise to the solution of ((2-((allyloxy)carbonyl)benzo[b]thiophen-5-yl)methyl)phosphonic acid (200 mg, 0.64 mmol, 1.0 eq.) in dry DCM (6 mL) and DMF (1 drop) at 0° C. The reaction was allowed to warm to 40° C., then stirred for additional 1-2 h. The reaction was monitored by pipetting out a small amount of crude sample and quenching it with MeOH to ensure bis-Cl phosphoryl chloride allyl 5-((dichlorophosphoryl)methyl)benzo[b]thiophene-2-carboxylate had been formed completely (bis-methoxy phosphonate was observed by LCMS). After completion, the excess of oxalyl chloride and solvent were removed under reduced pressure, and the residue was re-dissolved in anhydrous DCM (5 mL). To the solution was then added BnNH₂ (64.8 mg, 0.64 mmol, 1.0 eq.) in anhydrous DCM (2 mL) and Et₃N (194.32 mg, 1.92 mmol, 3.0 eq.) at −40° C. The reaction was monitored by pipetting out a small amount of crude sample and quenching with MeOH to ensure most of the product was allyl 5-(((benzylamino)chlorophosphoryl)methyl)benzo[b]thiophene-2-carboxylate (mono-methoxy phosphonate was observed by LCMS). To the solution was then added isopropyl L-alaninate (107.2 mg, 0.64 mmol, 1.0 eq.) in anhydrous DCM (2 mL) at −40° C. The reaction was allowed to warm to room temperature and stirred for additional 2 h. After completion, the reaction was quenched by adding H₂O (10 mL) and extracted with DCM (10 mL×3). The organic layers were combined and washed with brine (20 mL), dried over anhydrous Na₂SO₄, and concentrated under reduced pressure. The residue was purified by flash column chromatography on silica gel to afford allyl 5-(((benzylamino)(((S)-1-isopropoxy-1-oxopropan-2-yl)amino)phosphoryl)methyl)benzo[b]thiophene-2-carboxylate (100 mg, 194.33 μmol, 30% yield). LCMS (ESI): m/z=515.2 [M+H]⁺.

Step 2: Preparation of 5-(((benzylamino)(((S)-1-isopropoxy-1-oxopropan-2-yl)amino)phosphoryl)methyl)benzo[b]thiophene-2-carboxylic acid

A solution of allyl 5-(((benzylamino)(((S)-1-isopropoxy-1-oxopropan-2-yl)amino)phosphoryl)methyl)benzo[b]thiophene-2-carboxylate (100 mg, 194 μmol, 1 eq.), Pd(PPh₃)₄ (22.47 mg, 19.4 μmol, 0.1 eq.) and pyrrolidine (13.8 mg, 194 μmol, 1 eq.) in DCM (3 mL) was stirred at room temperature for 1 hr. After completion, the reaction mixture was concentrated under reduced pressure. The residue was purified by flash column chromatography on silica gel to afford 5-(((benzylamino)(((S)-1-isopropoxy-1-oxopropan-2-yl)amino)phosphoryl)methyl)benzo[b]thiophene-2-carboxylic acid (70 mg, 147.5 μmol, 76% yield). LCMS (ESI): m/z=475.1 [M+H]⁺.

Synthesis of 4-nitrophenyl 5-((bis(((propoxycarbonyl)oxy)methoxy)phosphoryl)difluoromethyl)benzo[b]thiophene-2-carboxylate

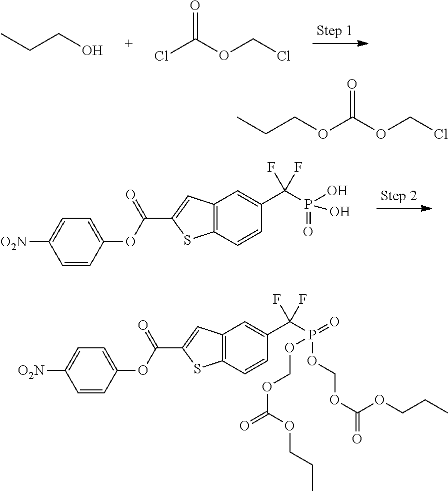

Step 1: Preparation of chloromethyl propyl carbonate

A solution of propan-1-ol (1.24 mL, 16.5 mmol, 1 eq) and chloroacetyl chloride (1.46 mL, 16.5 mmol, 1 eq) in diethyl ether (50 mL) was cooled down to 0° C. under nitrogen. Pyridine (1.32 mL, 16.5 mmol, 1.0 eq) was added dropwise and then the reaction mixture was stirred for 15 min. at 0° C. followed by 3 h at room temperature. The white suspension was filtered and rinsed with diethyl ether (20 mL). The filtrate was washed with 1 N HCl (20 mL) and water (2×15 mL) and then dried over anhydrous sodium sulfate, filtered and concentrated under reduced pressure to give chloromethyl propyl carbonate (1.26 g, 8.25 mmol, 50.1% yield) as a clear oil. ¹H NMR (400 MHz, CDCl₃) δ 5.74 (s, 2H), 4.25-4.17 (m, 2H), 1.74 (sxt, J=7.1 Hz, 2H), 1.02-0.94 (m, 3H).

Step 2: Preparation of 4-nitrophenyl 5-((bis(((((1-methoxy-2-methylpropan-2-yl)oxy)carbonyl)oxy)methoxy)phosphoryl)difluoromethyl)benzo[b]thiophene-2-carboxylate

Sodium hydroxide (37.1 mg, 930 μmol, 2 eq) in H₂O (1 mL) was added dropwise to a stirred suspension of (difluoro(2-((4-nitrophenoxy)carbonyl)benzo[b]thiophen-5-yl)methyl)phosphonic acid (200 mg, 465 μmol, 1 eq) in water (4 mL). When the mixture reached pH˜8, product started to precipitate and silver nitrate (236 mg, 1.39 mmol, 3 eq) was added in one portion. After 2 h at 0° C., the yellow precipitate was collected by filtration, washed with water and dried under vacuum. The powder was suspended in dry toluene (10 mL), and chloromethyl propyl carbonate (282 mg, 1.85 mmol, 4 eq) was added. The mixture was stirred for 18 h at room temperature. The crude was directly purified by normal phase chromatography using a 24 g silica gel cartridge eluting with a gradient of EtOAc in heptane (0 to 60% in 18 CV) to give 4-nitrophenyl 5-((bis(((((1-methoxy-2-methylpropan-2-yl)oxy)carbonyl)oxy)methoxy)phosphoryl)difluoromethyl)benzo[b]thiophene-2-carboxylate (10 mg, 15.1 μmol, 3.25% yield) as a white semi-solid: LCMS: m/z=662.2 [M+H]⁺; ¹H NMR (400 MHz, DMSO-d₆) δ 8.64 (s, 1H), 8.43-8.27 (m, 4H), 7.72-7.65 (m, 3H), 5.75-5.65 (m, 4H), 4.12-4.02 (m, 4H), 1.67-1.55 (m, 4H), 0.90-0.84 (m, 6H).

Synthesis of 4-nitrophenyl 5-((bis(((((1-methoxy-2-methylpropan-2-yl)oxy)carbonyl)oxy)methoxy)phosphoryl)difluoromethyl)benzo[b]thiophene-2-carboxylate

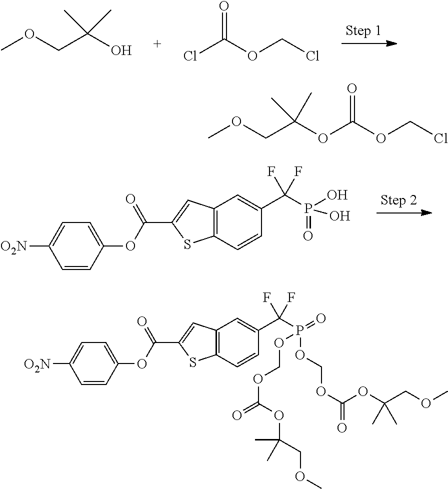

Step 1: Preparation of chloromethyl 1-methoxy-2-methylpropan-2-yl carbonate

A solution of 1-methoxy-2-methylpropan-2-ol (2 g, 19.2 mmol, 1 eq) and chloroacetyl chloride (1.7 mL, 19.2 mmol, 1 eq) in diethyl ether (60 mL) was cooled down to 0° C. under nitrogen. Pyridine (1.53 mL, 19.2 mmol, 1.0 eq) was added dropwise and then the reaction mixture was stirred for 15 min. at 0° C. followed by 3 h at room temperature. The white suspension was filtered and rinsed with diethyl ether (20 mL). The filtrate was washed with 1 N HCl (30 mL) and water (2×30 mL) and then dried over anhydrous sodium sulfate, filtered and concentrated under reduced pressure to give chloromethyl 1-methoxy-2-methylpropan-2-yl carbonate (860 mg, 4.37 mmol, 22.8% yield) as a clear oil. ¹H NMR (400 MHz, CDCl₃) δ 7.20-7.17 (m, 2H), 5.04-5.00 (m, 2H), 4.90 (s, 3H), 3.03-2.99 (m, 6H).

Step 2: Preparation of 4-nitrophenyl 5-((bis(((((1-methoxy-2-methylpropan-2-yl)oxy)carbonyl)oxy)methoxy)phosphoryl)difluoromethyl)benzo[b]thiophene-2-carboxylate

Sodium hydroxide (37.1 mg, 930 μmol, 2 eq) in H₂O (1 mL) was added dropwise to a stirred suspension of (difluoro(2-((4-nitrophenoxy)carbonyl)benzo[b]thiophen-5-yl)methyl)phosphonic acid (200 mg, 465 μmol, 1 eq) in water (5 mL). When the mixture reached pH˜8, product started to precipitate and silver nitrate (236 mg, 1.39 mmol, 3 eq) was added in one portion. After 2 h at 0° C., the white precipitate was collected by filtration, washed with water and dried under vacuum. The powder was suspended in dry toluene (10 mL), and chloromethyl 1-methoxy-2-methylpropan-2-yl carbonate (318 mg, 1.62 mmol, 3.5 eq) was added. The mixture was stirred for 18 h at 50° C. The crude mixture was directly purified by normal phase chromatography using a 24 g silica gel cartridge eluting with a gradient of EtOAc in heptane (0 to 60% in 18 CV) to give 4-nitrophenyl 5-((bis(((((1-methoxy-2-methylpropan-2-yl)oxy)carbonyl)oxy)methoxy)phosphoryl)difluoromethyl)benzo[b]thiophene-2-carboxylate (51.0 mg, 68.0 μmol, 14.6% yield) as a white semi-solid. ¹H NMR (400 MHz, CDCl₃) δ 8.38-8.33 (m, 3H), 8.24 (s, 1H), 8.03 (d, J=8.8 Hz, 1H), 7.76 (d, J=8.8 Hz, 1H), 7.51-7.47 (m, 2H), 5.74-5.62 (m, 4H), 3.53-3.50 (m, 4H), 3.41-3.38 (m, 6H), 1.51 (s, 12H).

Synthesis of 4-nitrophenyl 5-((bis(((dipropylcarbamoyl)oxy)methoxy)phosphoryl)difluoromethyl)benzo[b]thiophene-2-carboxylate

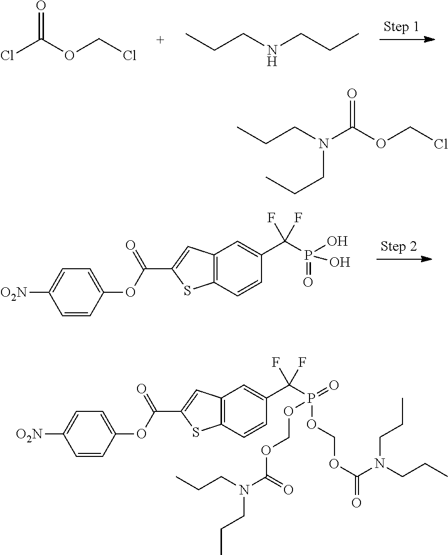

Step 1: Preparation of chloromethyl dipropylcarbamate

Dipropylamine (5.85 g, 57.9 mmol, 2.5 eq) was added to a solution of chloroacetyl chloride (2.06 mL, 23.2 mmol, 1 eq) in hexanes (30 mL) at 0° C. under nitrogen and stirred at the same temperature for 30 min. The reaction mixture was then diluted with EtOAc (50 mL). The phases were separated and the organic layer was washed successively with 1 N hydrochloric acid (50 mL), water and brine. The organic layer was dried over anhydrous sodium sulfate, then the solvent was evaporated under reduced pressure. The product was purified by flash-chromatography using an 80 g silica gel cartridge eluting with a gradient of 0-10% EtOAc in heptane. The pure combined fractions were dried under reduced pressure to give chloromethyl dipropylcarbamate (2.89 g, 14.9 mmol, 64.3% yield) as a colorless oil. LCMS: m/z=194.2 [M+H]⁺; ¹H NMR (400 MHz, CDCl₃) δ 5.75 (s, 2H), 3.21-3.11 (m, 4H), 1.59-1.47 (m, 4H), 0.87-0.82 (m, 6H).

Step 2: Preparation of 4-nitrophenyl 5-((bis(((dipropylcarbamoyl)oxy)methoxy)phosphoryl)difluoromethyl)benzo[b]thiophene-2-carboxylate

Sodium hydroxide (37.1 mg, 930 μmol, 2 eq) in H₂O (1 mL) was added dropwise to a stirred suspension of (difluoro(2-((4-nitrophenoxy)carbonyl)benzo[b]thiophen-5-yl)methyl)phosphonic acid (200 mg, 465 μmol, 1 eq) in H₂O (5 mL). When the mixture reached pH˜8, product started to precipitate and silver nitrate (236 mg, 1.39 mmol, 3 eq) was added in one portion. After 2 h at 0° C., the yellow precipitate was collected by filtration, washed with water, dried under vacuum. The powder was suspended in dry toluene (10 mL), and chloromethyl dipropylcarbamate (313 mg, 1.62 mmol, 3.5 eq) was added. The mixture was stirred for 18 h at room temperature. The crude was directly purified by normal phase chromatography using a 12 g silica gel cartridge eluting with a gradient of EtOAc in heptane (0 to 60% in 18 CV) to give 4-nitrophenyl 5-((bis(((dipropylcarbamoyl)oxy)methoxy)phosphoryl)difluoromethyl)benzo[b]thiophene-2-carboxylate (110 mg, 147 μmol, 31.8% yield) as a clear oil. ¹H NMR (400 MHz, DMSO-d₆) δ 8.65-8.56 (m, 1H), 8.41-8.21 (m, 4H), 7.76-7.59 (m, 3H), 5.77-5.60 (m, 4H), 3.17-2.99 (m, 8H), 1.53-1.38 (m, 8H), 0.85-0.75 (m, 12H).

Synthesis of 4-nitrophenyl 5-((bis(1-((propoxycarbonyl)oxy)ethoxy)phosphoryl)difluoromethyl)benzo[b]thiophene-2-carboxylate

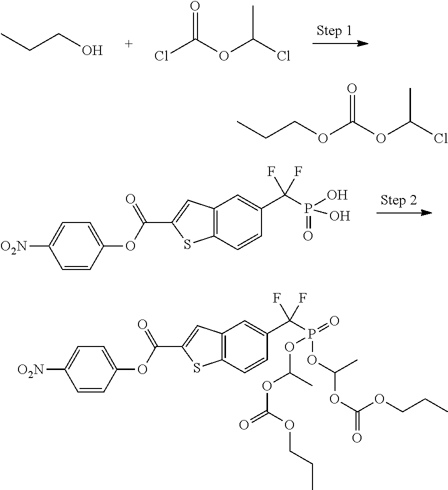

Step 1: Preparation of 1-chloroethyl propyl carbonate

A solution of propan-1-ol (2 g, 33.2 mmol, 1 eq) and 1-chloroethyl chloroformate (4.74 g, 33.2 mmol, 1 eq) in diethyl ether (100 mL) was cooled to 0° C. under nitrogen. Pyridine (2.66 mL, 33.2 mmol, 1.0 eq) was added dropwise and then the reaction mixture was stirred for 15 min. at 0° C. followed by 3 h at room temperature. The white suspension was filtered and rinsed with diethyl ether (20 mL). The filtrate was washed with 1 N HCl (20 mL) and water (2×15 mL) and then dried over anhydrous sodium sulfate, filtered and concentrated under reduced pressure to give 1-chloroethyl propyl carbonate (4.62 g, 27.7 mmol, 83.5% yield) as a clear oil. ¹H NMR (400 MHz, CDCl₃) δ 6.44 (q, J=5.8 Hz, 1H), 4.24-4.10 (m, 2H), 1.84 (d, J=5.9 Hz, 3H), 1.78-1.66 (m, 2H), 1.01-0.94 (m, 3H).

Step 2: Preparation of 4-nitrophenyl 5-((bis(1-((propoxycarbonyl)oxy)ethoxy)phosphoryl)difluoromethyl)benzo[b]thiophene-2-carboxylate

Sodium hydroxide (37.1 mg, 930 μmol, 2 eq) in H₂O (1 mL) was added dropwise to a stirred suspension of (difluoro(2-((4-nitrophenoxy)carbonyl)benzo[b]thiophen-5-yl)methyl)phosphonic acid (200 mg, 465 μmol, 1 eq) in H₂O (4 mL). When the mixture became clear (pH˜8), silver nitrate (236 mg, 1.39 mmol, 2 eq) was added in one portion. After 2 h at 0° C., the yellow precipitate was collected by filtration and dried under vacuum. The powder was suspended in dry toluene (10 mL) and 1-chloroethyl propyl carbonate (269 mg, 1.62 mmol, 3.5 eq) was added. The mixture was stirred for 18 h at room temperature. The crude was directly purified by normal phase chromatography using a 12 g silica gel cartridge eluting with a gradient of EtOAc in heptane (0 to 60% in 18 CV) to give 4-nitrophenyl 5-((bis(1-((propoxycarbonyl)oxy)ethoxy)phosphoryl)difluoromethyl)benzo[b]thiophene-2-carboxylate (113 mg, 163 μmol, 35.3% yield) as a clear oil. LCMS: m/z=712.2 (M+Na)⁺.

Synthesis of (E)-3-(3-((diethoxyphosphoryl)difluoromethyl)phenyl)-2-methylacrylic acid

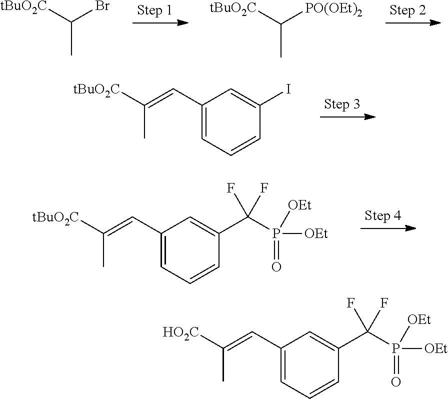

Step 1: Preparation of tert-butyl 2-(diethoxyphosphoryl)propanoate

A mixture of tert-butyl 2-bromopropanoate (3 g, 14.3 mmol, 1 eq.) and ethan-2-ylium-1-yl diethyl phosphite (2.82 g, 17.1 mmol, 1.2 eq.) was stirred at 110° C. for 16 h. After completion, the reaction mixture was concentrated under reduced pressure. The residue was purified by flash column chromatography on silica gel to afford tert-butyl 2-(diethoxyphosphoryl)propanoate (3.0 g, 11.3 mmol, 79% yield) as a white solid. LCMS (ESI): m/z=267 [M+H]⁺.

Step 2: Preparation of tert-butyl (E)-3-(3-iodophenyl)-2-methylacrylate

To a solution of tert-butyl 2-(diethoxyphosphoryl)propanoate (1.14 g, 4.30 mmol, 1 eq.) in THF (10 mL) was added n-BuLi (1.72 mL, 4.30 mmol, 1.0 eq.) at −78° C. under nitrogen and the resulting mixture was stirred at this temperature for 0.5 hr. Then, a solution of 3-iodobenzaldehyde (1.0 g, 4.30 mmol, 1 eq.) in THF (5 mL) was added, after addition, the reaction mixture was allowed to warm to room temperature and stirred for 14 h. After completion, the reaction mixture was quenched by adding H₂O (20 mL), then extracted with EtOAc (15 mL×3). The organic layers were combined and washed with brine (15 mL), dried over anhydrous Na₂SO₄, and concentrated under reduced pressure. The residue was purified by flash column chromatography on silica gel to afford tert-butyl (E)-3-(3-iodophenyl)-2-methylacrylate (739.6 mg, 2.15 mmol, 50% yield) as a white solid. LC-MS (ESI) m/z=345 [M+H]⁺.

Step 3: Preparation of tert-butyl (E)-3-(3-((diethoxyphosphoryl)difluoromethyl)phenyl)-2-methylacrylate

A solution of diethyl (bromodifluoromethyl)phosphonate (1.08 g, 4.06 mmol, 2 eq.) zinc (265 mg, 4.06 mmol, 2 eq.) in DMAc (7 mL) was stirred at 60° C. for 1 h under nitrogen atmosphere. Then CuBr (582 mg, 4.06 mmol, 2 eq.) was added and the mixture was stirred at 60° C. for another 1 hr. Tert-butyl (E)-3-(3-iodophenyl)-2-methylacrylate (700 mg, 2.03 mmol, 1 eq.) was added and the mixture was stirred at 60° C. for another 12 h. After completion, the suspension was filtered through a pad of Celite®, the filtrate was concentrated under reduced pressure. The residue was purified by flash column chromatography on silica gel to give tert-butyl (E)-3-(3-((diethoxyphosphoryl)difluoromethyl)phenyl)-2-methylacrylate (180 mg, 445 mol, 22% yield) as a yellow oil. LCMS (ESI): m/z=405 [M+H]⁺.

Step 4: Preparation of (E)-3-(3-((diethoxyphosphoryl)difluoromethyl)phenyl)-2-methylacrylic acid

A solution of tert-butyl (E)-3-(3-((diethoxyphosphoryl)difluoromethyl)phenyl)-2-methylacrylate (180 mg, 445 mol, 1 eq.) in a mixture of DCM (8 mL) and TFA (4 mL) was stirred at room temperature for 1 hr. After completion, the reaction mixture was concentrated under reduced pressure to give (E)-3-(3-((diethoxyphosphoryl)difluoromethyl)phenyl)-2-methylacrylic acid (154 mg, quant) as a white solid, which was used in next step directly without further purification. LCMS (ESI): m/z=349 [M+H]⁺.

Synthesis of (E)-3-(3-((diethoxyphosphoryl)difluoromethyl)phenyl)but-2-enoic acid

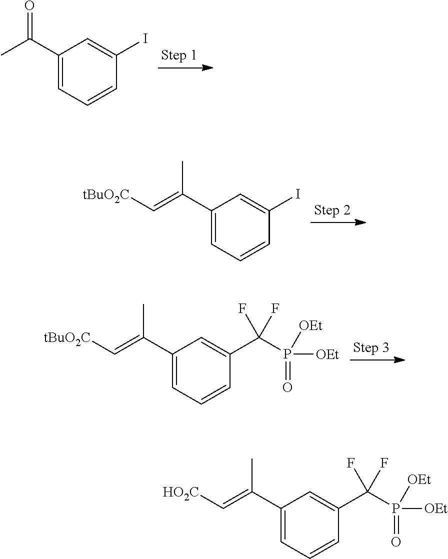

Step 1: Preparation of tert-butyl (E)-3-(3-iodophenyl)but-2-enoate

n-BuLi (1.72 mL, 4.06 mmol, 1.0 eq.) was added to tert-butyl 2-(diethoxyphosphoryl)acetate (1.02 g, 4.06 mmol, 1 eq.) in THF (20 mL) at −78° C., the mixture was stirred at −78° C. for 0.5 hr. Then a solution of 1-(3-iodophenyl)ethan-1-one (1 g, 4.06 mmol, 1 eq.) in THF (5 mL) was added dropwise to the reaction. After addition, the reaction mixture was allowed to warm to room temperature and stirred for 18 h. After completion, the reaction mixture was quenched by adding H₂O (20 mL), then extracted with EtOAc (20 mL×3). The organic layers were combined and washed with brine (15 mL), dried over anhydrous Na₂SO₄, and concentrated under reduced pressure. The residue was purified by flash column chromatography on silica gel to afford tert-butyl (E)-3-(3-iodophenyl)but-2-enoate (800 mg, 2.32 mmol, 58% yield) as an oil. ¹H NMR (400 MHz, CDCl₃) δ 7.79 (t, J=1.6 Hz, 1H), 7.66 (d, J=7.9 Hz, 1H), 7.40 (d, J=7.9 Hz, 1H), 7.09 (t, J=7.9 Hz, 1H), 6.01 (d, J=1.2 Hz, 1H), 2.49 (d, J=1.2 Hz, 3H), 1.52 (s, 9H).

Step 2: Preparation of tert-butyl (E)-3-(3-((diethoxyphosphoryl)difluoromethyl)phenyl)but-2-enoate

To a stirred suspension of Zn (301 mg, 4.64 mmol, 2 eq.) in dry DMAc (2 mL) was added slowly a solution of diethyl (bromodifluoromethyl)phosphonate (1.23 g, 4.64 mmol, 2 eq.) in DMAc (2 mL) under nitrogen atmosphere. The reaction mixture was stirred at 45° C. for 2 h and then CuBr (665 mg, 4.64 mmol, 2 eq.) was added and the resulting mixture was stirred at room temperature for 45 minutes. A suspension of tert-butyl (E)-3-(3-iodophenyl)but-2-enoate (800 mg, 2.32 mmol, 1 eq.) in DMAc (3 mL) was added into the reaction mixture. The mixture was stirred at 45° C. for 24 h under nitrogen atmosphere. The reaction mixture was then partitioned between water and ether. The mixture was passed through celite and extracted with ether. The organic extracts were washed with brine and dried over anhydrous Na₂SO₄. The solvent was removed in vacuo, and the residue was purified by flash column chromatography on silica to give tert-butyl (E)-3-(3-((diethoxyphosphoryl)difluoromethyl)phenyl)but-2-enoate (350 mg, 0.87 mmol, 37% yield) as an oil. ¹H NMR (400 MHz, CDCl₃) δ 7.67 (d, J=13.2 Hz, 1H), 7.63-7.54 (m, 2H), 7.45 (t, J=7.7 Hz, 1H), 6.07 (d, J=1.2 Hz, 1H), 4.27-4.11 (m, 4H), 2.54 (d, J=1.0 Hz, 3H), 1.52 (s, 9H), 1.32 (t, J=7.1 Hz, 6H).

Step 3: Preparation of (E)-3-(3-((diethoxyphosphoryl)difluoromethyl)phenyl)but-2-enoic acid

To a solution of tert-butyl (E)-3-(3-((diethoxyphosphoryl)difluoromethyl)phenyl)but-2-enoate (350 mg, 0.87 mmol, 1 eq.) in DCM (4 mL) was added TFA (6.13 g, 53.8 mmol, 62.2 eq.) at room temperature and the resulting mixture was stirred at room temperature for 18 h. After completion, the reaction mixture was concentrated under reduced pressure to afford (E)-3-(3-((diethoxyphosphoryl)difluoromethyl)phenyl)but-2-enoic acid (250 mg, 0.72 mmol, 83% yield) as an oil, which was used in next step without further purification. LC-MS (ESI) m/z=347.2 [M−H]⁻.

Synthesis of perfluorophenyl 7-((bis(2-(butyrylthio)ethoxy)phosphoryl)difluoromethyl)-2-naphthoate and perfluorophenyl 7-(((2-(butyrylthio)ethoxy)(hydroxy)phosphoryl)difluoromethyl)-2-naphthoate

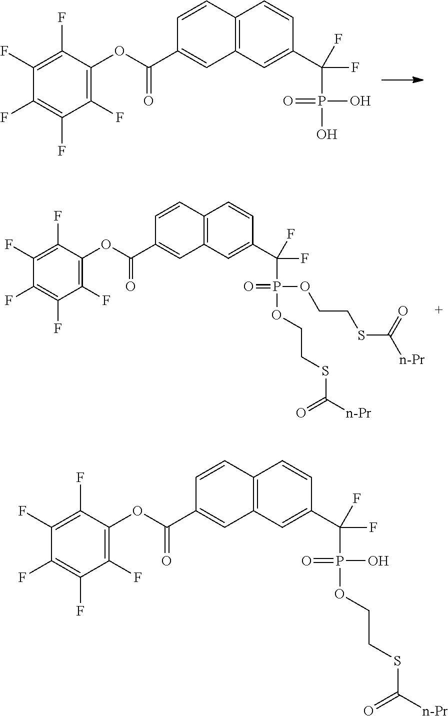

To a solution of (difluoro(7-((perfluorophenoxy)carbonyl)naphthalen-2-yl)methyl)phosphonic acid (1 g, 2.10 mmol, 1 eq.) in dry DCM (18 mL) was added oxalyl chloride (266 mg, 2.13 mmol, 1.0 eq.) dropwise followed with DMF (155 mg, 2.13 mmol, 1.0 eq.) at 25° C. The reaction mixture was warmed to 40° C., then stirred for an additional 1.2 h under reflux. The reaction was monitored by pipetting out a small amount of crude sample and quenching it with MeOH to ensure bis-Cl phosphoryl chloride had been formed completely (bis-methoxy phosphonate was observed by LCMS). After completion, the excess oxalyl chloride and solvent were removed under reduced pressure. The residue was re-dissolved in anhydrous DCM (14 mL) then added to a mixture of S-(2-hydroxyethyl) butanethioate (641 mg, 4.33 mmol, 2.2 eq.) and triethylamine (198 mg, 1.97 mmol, 1.0 eq.) in anhydrous DCM (15 mL) at 0° C. The reaction was allowed to warm to room temperature and stirred for an additional 12 h. The reaction progress was monitored by LCMS, and after completion, the reaction was quenched by adding H₂O (10 mL) and concentrated under reduced pressure. The residue was purified by C18 column chromatography to afford two products: perfluorophenyl 7-((bis(2-(butyrylthio)ethoxy)phosphoryl)difluoromethyl)-2-naphthoate (680 mg, 1.13 mmol, 58% yield) as a white solid. LCMS (ESI): m/z=729 [M+H]⁺ and perfluorophenyl 7-(((2-(butyrylthio)ethoxy)(hydroxy)phosphoryl)difluoromethyl)-2-naphthoate (65.0 mg, 89.2 mol) as a white solid. LCMS (ESI): m/z=599 [M+H]⁺.

Synthesis of perfluorophenyl 7-(((2-(butyrylthio)ethoxy)(hydroxy)phosphoryl)difluoromethyl)-2-naphthoate

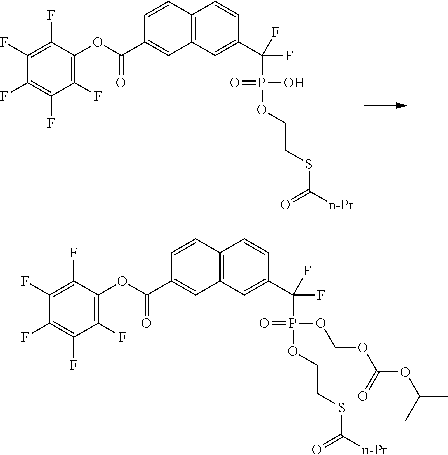

To a solution of perfluorophenyl 7-(((2-(butyrylthio)ethoxy)(hydroxy)phosphoryl)difluoromethyl)-2-naphthoate (300 mg, 501 mol, 1 eq.) in a mixture of deionized H₂O (8 mL) and THF (2 mL) was added Amberlite IR120® resin (Na⁺ form) (233 mg, 751 mol, 1.5 eq.). The resulting mixture was stirred at room temperature for 1 h, then the excess of the resin was removed by filtration. AgNO₃ (127 mg, 751 mol, 1.5 eq.) in deionized H₂O (2 mL) was then added to the resulting solution. After addition, the resulting mixture was stirred at room temperature for an additional 1 hr. During this period, the silver salt formed as a white precipitate, which was collected via filtration. The filter cake was washed with cool H₂O (2 mL×3), and the silver salt was further dried under reduced pressure to get a dry powder, which was pure enough for next step without further purification.

The isolated mono-Ag salt was suspended in ACN (8 mL), and iodomethyl isopropyl carbonate (244 mg, 1.00 mmol, 2 eq.) was added dropwise. After addition, the resulting mixture was stirred at 40° C. for an additional 12 h. The reaction progress was monitored by LCMS, and after completion, the unreacted silver salt was recovered by filtration, and the filtrate was concentrated under reduced pressure. The resulting residue was purified by prep-TLC to give perfluorophenyl 7-(((2-(butyrylthio)ethoxy)(((isopropoxycarbonyl)oxy)methoxy)phosphoryl)difluoromethyl)-2-naphthoate (30 mg, 41.9 mol, 8%) as colorless oil. LCMS (ESI) m/z=737 [M+Na]⁺.

Synthesis of 5-((bis(((S)-1-isopropoxy-1-oxopropan-2-yl)amino)phosphoryl)methyl)benzo[b]thiophene-2-carboxylic acid

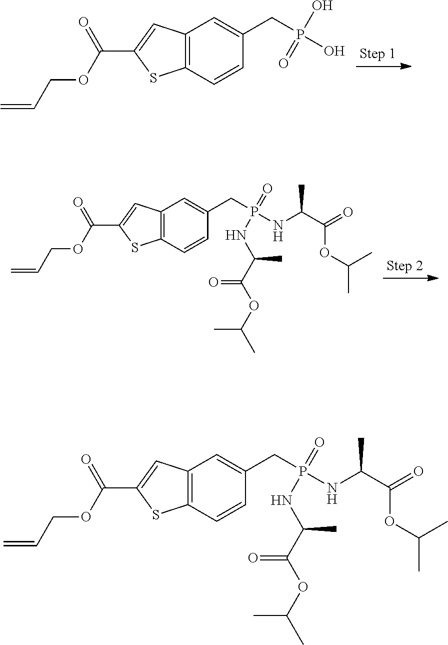

Step 1: Preparation of diisopropyl 2,2′-((((2-((allyloxy)carbonyl)benzo[b]thiophen-5-yl)methyl)phosphoryl)bis(azanediyl))(2S,2'S)-dipropionate

To a solution of ((2-((allyloxy)carbonyl)benzo[b]thiophen-5-yl)methyl)phosphonic acid (300 mg, 960 μmol, 1.0 eq.) in dry DCM (10 mL) and DMF (cat.) at 0° C. was added oxalyl chloride (609 mg, 4.80 mmol, 5.0 eq.) dropwise. The reaction mixture was allowed to warm to 40° C., then stirred for additional 1-2 h. The reaction was monitored by pipetting out a small amount of crude sample and quenching it with MeOH to ensure bis-Cl phosphoryl chloride had been formed completely (bis-methoxy phosphonate was observed by LCMS). After completion, the excess of oxalyl chloride and solvent were removed under reduced pressure, and the residue was re-dissolved in anhydrous DCM (5 mL). This solution was then added to a mixture of isopropyl L-alaninate (502 mg, 3.83 mmol, 4.0 eq.) and N,N-diisopropylethylamine (620 mg, 4.80 mmol, 5.0 eq.) in anhydrous DCM (10 mL) at 0° C. The reaction was allowed to warm to room temperature and stirred for additional 18 h. The progress was monitored by LCMS. After completion, the reaction was quenched by adding H₂O (10 mL) and extracted with DCM (10 mL×3). The organic layers were combined and washed with brine (20 mL), dried over anhydrous Na₂SO₄, and concentrated under reduced pressure. The residue was purified by flash column chromatography on silica gel to afford diisopropyl 2,2′-((((2-((allyloxy)carbonyl)benzo[b]thiophen-5-yl)methyl)phosphoryl)bis(azanediyl))(2S,2'S)-dipropionate (70.0 mg, 129 μmol, 14% yield). LCMS (ESI): m/z=539.2 [M+H]⁺.

Step 2: Preparation of 5-((bis(((S)-1-isopropoxy-1-oxopropan-2-yl)amino)phosphoryl)methyl)benzo[b]thiophene-2-carboxylic acid

To a solution of diisopropyl 2,2′-((((2-((allyloxy)carbonyl)benzo[b]thiophen-5-yl)methyl)phosphoryl)bis(azanediyl))(2S,2'S)-dipropionate (70 mg, 129 μmol, 1.0 eq.) in DCM (1 mL) was added pyrrolidine (9.17 mg, 129 μmol, 1.0 eq.) and Pd(PPh₃)₄ (14.9 mg, 12.9 μmol, 0.1 eq.) under N₂, and the resulting mixture was stirred at room temperature for 2 h. After completion, the reaction mixture was cooled down in an ice-bath, then neutralized carefully with HCl (aq. 1M) until the pH was adjusted to pH=4-6. The resulting mixture was extracted with DCM (10 mL×3), and the combined organic layers were washed with brine (10 mL×2), dried over anhydrous Na₂SO₄, then concentrated under reduced pressure. The residue was purified by Biotage® C18 column chromatography to give 5-((bis(((S)-1-isopropoxy-1-oxopropan-2-yl)amino)phosphoryl)methyl)benzo[b]thiophene-2-carboxylic acid (70.0 mg, 140 μmol, 108%, 60% purity) as a colorless oil. LCMS (ESI): m/z=499.2 [M+H]⁺.

Synthesis of 5-(difluoro(((2-isopropoxy-2-oxoethyl)amino)(phenoxy)phosphoryl)methyl)benzo[b]thiophene-2-carboxylic acid

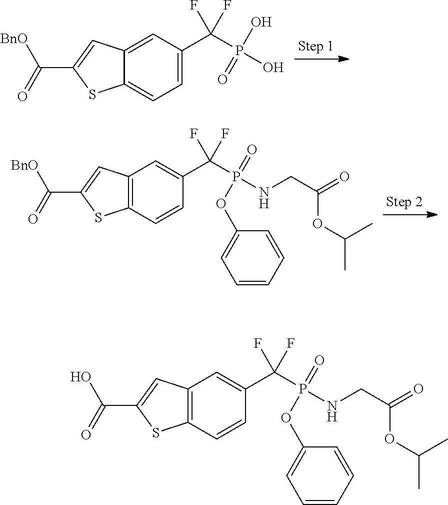 [SMILES: O=C(OCc1ccccc1)c1cc2cc(C(F)(F)P(=O)(O)O)ccc2s1 ; CC(C)OC(=O)CNP(=O)(Oc1ccccc1)C(F)(F)c1ccc2sc(C(=O)OCc3ccccc3)cc2c1 ; CC(C)OC(=O)CNP(=O)(Oc1ccccc1)C(F)(F)c1ccc2sc(C(=O)O)cc2c1]

Step 2: Preparation of 5-(difluoro(((2-isopropoxy-2-oxoethyl)amino)(phenoxy)phosphoryl)methyl)benzo[b]thiophene-2-carboxylic acid

To a solution of benzyl 5-(difluoro(((2-isopropoxy-2-oxoethyl)amino)(phenoxy)phosphoryl)methyl)benzo[b]thiophene-2-carboxylate (90 mg, 156 μmol, 1 eq) in anhydrous tetrahydrofuran (10 mL) under nitrogen was added 10% palladium on carbon (50% wet) (166 mg, 78.0 μmol, 0.5 eq). Hydrogen was bubbled for 5 min. and the reaction mixture was stirred at room temperature for 18 h under hydrogen (1 atm). Nitrogen was bubbled int the mixture and it was then filtered over Celite (rinsing with 2-MeTHF) and concentrated under reduced pressure. The isolated product 5-(difluoro(((2-isopropoxy-2-oxoethyl)amino)(phenoxy)phosphoryl)methyl)benzo[b]thiophene-2-carboxylic acid was used without further purification. LCMS: m/z 484.2 [M+H]⁺.

The following linkers in Table 16 were prepared according to the procedure described for the synthesis of 5-(difluoro(((2-isopropoxy-2-oxoethyl)amino)(phenoxy)phosphoryl)methyl)benzo[b]thiophene-2-carboxylic acid utilizing the appropriate starting materials.

TABLE 16

| Name | Structure | LCMS | NMR |
|---|---|---|---|
| 5-(difluoro((((S)-3- isopropoxy-2- methyl-3- oxopropyl)amino) (phenoxy)phosphoryl) methyl)benzo[b] thiophene-2- carboxylic acid | 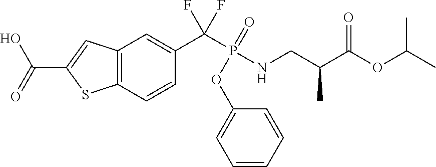 [SMILES: CC(C)OC(=O)[C@@H](C)CNP(=O)(Oc1ccccc1)C(F)(F)c1ccc2sc(C(=O)O)cc2c1] | 512.4 [M + H]⁺ | |
| 5-(difluoro((((S)-1- isopropoxy-1-oxo- 3-phenylpropan-2- yl)amino)(phenoxy) phosphoryl)methyl) benzo[b]thiophene- 2-carboxylic acid | 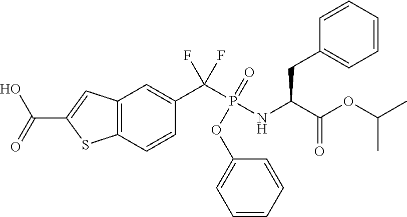 [SMILES: CC(C)OC(=O)[C@H](Cc1ccccc1)NP(=O)(Oc1ccccc1)C(F)(F)c1ccc2sc(C(=O)O)cc2c1] | 574.2 [M + H]⁺ | |
| 5-(((((S)-1- (benzyloxy)-1-oxo- 4-phenylbutan-2- yl)amino)(phenoxy) phosphoryl) difluoromethyl)benzo[b] | 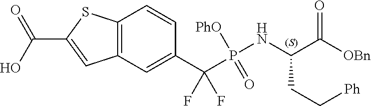 | 636.4 [M + H]⁺ | |
| thiophene-2- | | | |
| carboxylic acid | | | |
| 5-(((((S)-1- (benzyloxy)-4- methyl-1- oxopentan-2- yl)amino)(phenoxy) phosphoryl) difluoromethyl)benzo [b]thiophene-2- carboxylic acid | 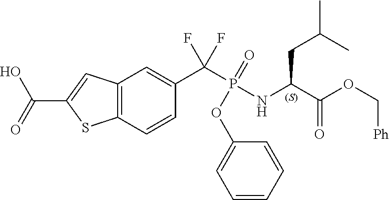 | 588.2 [M + H]⁺ | (400 MHZ, DMSO-d₆) δ 13.67 (br. s., 1H), 8.28- 8.13 (m, 3H), 7.73-7.63 (m, 1H), 7.42-7.27 (m, 7H), 7.23-7.06 (m, 3H), 6.79 (td, J = 14.9, 10.6 Hz, 1H), 5.09-4.88 (m, 2H), 3.88-3.64 (m, 1H), 1.49-1.28 (m, 3H), 0.72-0.54 (m, 6H). |

Synthesis of 4-nitrophenyl 5-(difluoro((2-isopropoxy-2-oxoethoxy)(phenoxy)phosphoryl)methyl)benzo[b]thiophene-2-carboxylate

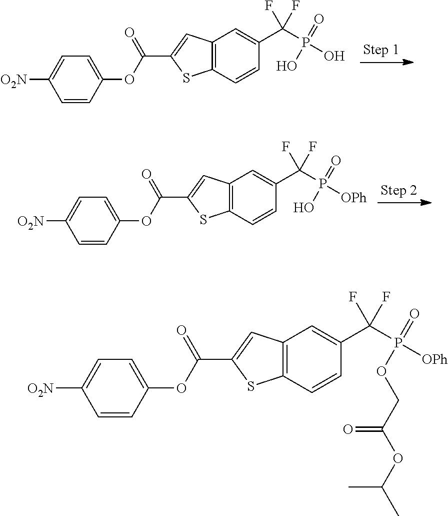 [SMILES: O=C(Oc1ccc([N+](=O)[O-])cc1)c1cc2cc(C(F)(F)P(=O)(O)O)ccc2s1 ; O=C(Oc1ccc([N+](=O)[O-])cc1)c1cc2cc(C(F)(F)P(=O)(O)Oc3ccccc3)ccc2s1 ; CC(C)OC(=O)COP(=O)(Oc1ccccc1)C(F)(F)c1ccc2sc(C(=O)Oc3ccc([N+](=O)[O-])cc3)cc2c1]

Step 1: Preparation of 4-nitrophenyl 5-(difluoro(hydroxy(phenoxy)phosphoryl)methyl)benzo[b]thiophene-2-carboxylate

To a solution of (difluoro(2-((4-nitrophenoxy)carbonyl)benzo[b]thiophen-5-yl)methyl)phosphonic acid (1 g, 2.32 mmol, 1 eq) in methylene chloride (6 mL) at 0° C. were added 2 drops of DMF (cat.) followed by dropwise addition of oxalyl chloride (1.18 mL, 13.9 mmol, 6 eq). The reaction was warmed up to room temperature and stirred for 2 h. The reaction was not soluble at first (white solid floating on surface) but became a clear solution upon adding oxalyl chloride and warming up to room temperature. The reaction was concentrated under reduced pressure and dried completely under high vacuum for 30 min. to give a yellow solid. Then, the latter was diluted in methylene chloride (10 mL) and cooled down to −78° C. A solution of phenol (218 mg, 2.32 mmol, 1 eq) and triethylamine (646 μL, 4.64 mmol, 2 eq) in DCM (1 mL) (dried on Na₂SO₄) was slowly added on the yellow solution over 5 min. The reaction mixture was stirred at −78° C. for 15 min., then warmed up to room temperature and stirred for 2 h. Water (1 mL) was added and the reaction mixture was concentrated under reduced pressure. The crude residue was purified by reverse phase chromatography on a 50 g C₁₈ cartridge eluting with a gradient of 5-100% MeCN in water (with 0.1% formic acid) to give 4-nitrophenyl 5-(difluoro(hydroxy(phenoxy)phosphoryl)methyl)benzo[b]thiophene-2-carboxylate (848 mg, 1.67 mmol, 72.4%) as a yellow solid. ¹H NMR (400 MHz, DMSO-d₆) δ 9.04 (br. s., 1H), 8.63 (s, 1H), 8.37 (d, J=9.0 Hz, 2H), 8.29 (s, 1H), 8.19 (d, J=8.8 Hz, 1H), 7.78 (d, J=8.8 Hz, 1H), 7.71 (d, J=9.0 Hz, 2H), 7.27-7.18 (m, 2H), 7.10 (d, J=8.3 Hz, 2H), 7.02-6.95 (m, 1H).

Step 2: Preparation of 4-nitrophenyl 5-(difluoro((2-isopropoxy-2-oxoethoxy)(phenoxy)phosphoryl)methyl)benzo[b]thiophene-2-carboxylate

To a solution of 4-nitrophenyl 5-(difluoro(hydroxy(phenoxy)phosphoryl)methyl)benzo[b]thiophene-2-carboxylate (100 mg, 197 μmol, 1 eq) in methylene chloride (5 mL) at 0° C. was added 2 drops of DMF (cat.) followed by dropwise addition of oxalyl chloride (168 μL, 1.97 mmol, 10 eq). The reaction was warmed up to room temperature and stirred for 18 h. The reaction mixture was concentrated under reduced pressure and dried completely under high vacuum. Then, the latter was diluted in methylene chloride (5 mL) and cooled down to 0° C. A solution of propan-2-yl 2-hydroxyacetate (30.2 mg, 256 μmol, 1.3 eq) and triethylamine (82.3 μL, 591 μmol, 3 eq) in DCM (1 mL) (dried on Na₂SO₄) was slowly added on the orange solution. The reaction mixture was stirred at 0° C. for 5 min., then warmed up to room temperature and stirred for 5 h. The reaction mixture was concentrated under reduced pressure. The crude residue was purified by reverse phase chromatography on a 50 g C18 cartridge eluting with 5-100% MeCN in basic water (NH₄HCO₃ 10 mM, pH=10) to give 4-nitrophenyl 5-(difluoro((2-isopropoxy-2-oxoethoxy)(phenoxy)phosphoryl)methyl)benzo[b]thiophene-2-carboxylate (65.0 mg, 107 μmol, 54.6%) as a yellow oil. LCMS: m/z=606.2 [M+H]⁺; ¹H NMR (400 MHz, DMSO-d₆) δ 8.66 (s, 1H), 8.46 (s, 1H), 8.41-8.35 (m, 3H), 7.83 (d, J=8.3 Hz, 1H), 7.72-7.67 (m, 2H), 7.44-7.38 (m, 2H), 7.28-7.20 (m, 3H), 5.02-4.94 (m, 1H), 4.91-4.73 (m, 2H), 1.18 (dd, J=6.4, 2.4 Hz, 6H).

The following linkers in Table 17 were prepared according to the procedure described for 4-nitrophenyl 5-(difluoro((2-isopropoxy-2-oxoethoxy)(phenoxy)phosphoryl)methyl)benzo[b]thiophene-2-carboxylate utilizing the appropriate starting materials.

TABLE 17

| Name | Structure | LCMS | NMR |
|---|---|---|---|
| 4-nitrophenyl 5- (difluoro((((S)-1- methoxy-1- oxopropan-2- yl)oxy)(phenoxy) phosphoryl)methyl) benzo[b]thiophene- 2-carboxylate | 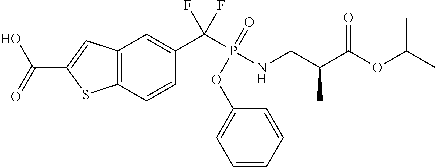 | 592.2 [M + H]⁺ | ¹H NMR (400 MHZ, DMSO-d₆) δ 8.68 (d, J = 6.4 Hz, 1H), 8.48-835 (m, 4H), 7.87-7.66 (m, 3H), 7.50-7.13 (m, 5H), 5.23-5.00 (m, 1H), 3.63 (s, 3H), 1.45-1.38 (m, 3H). |
| 5-(difluoro((((S)-1- isopropoxy-1-oxo- 3-phenylpropan-2- yl)oxy)(phenoxy) phosphoryl)methyl) benzo[b]thiophene- 2-carboxylic acid | 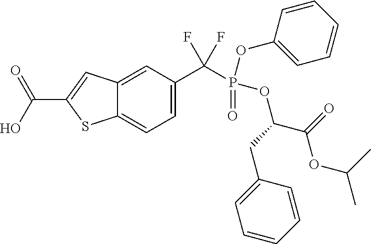 [SMILES: CC(C)OC(=O)[C@H](Cc1ccccc1)OP(=O)(Oc1ccccc1)C(F)(F)c1ccc2sc(C(=O)O)cc2c1] | 575.2 [M + H]⁺ | (400 MHZ, DMSO-d₆) δ 8.28-8.15 (m, 3H), 7.63- 7.46 (m, 1H), 7.41-7.30 (m, 2H), 7.28-7.17 (m, 4H), 7.16-7.07 (m, 3H), 7.01-6.94 (m, 1H), 5.29- 5.21 (m, 0.5H), 5.12-5.04 (m, 0.5H), 4.91-4.80 (m, 1H), 1.10-0.99 (m, 6H). |

Synthesis of 5-(((((S)-1-(benzyloxy)-1-oxopropan-2-yl)amino)(2-((3-methylbutanoyl)thio)ethoxy)phosphoryl)difluoromethyl)benzo[b]thiophene-2-carboxylic acid

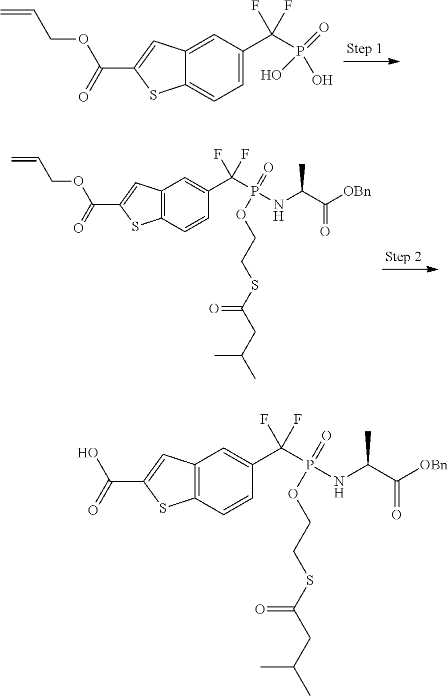

Step 1: Preparation of allyl 5-(((((S)-1-(benzyloxy)-1-oxopropan-2-yl)amino)(2-((3-methylbutanoyl)thio)ethoxy)phosphoryl)difluoromethyl)benzo[b]thiophene-2-carboxylate

To a solution of ((2-((allyloxy)carbonyl)benzo[b]thiophen-5-yl)difluoromethyl)phosphonic acid (200 mg, 0.5742 mmol, 1 eq) in methylene chloride (10 mL) at 0° C. was added 3 drops of DMF followed by dropwise addition of oxalyl chloride (147 μL, 1.72 mmol, 3 eq). The reaction was warmed up to room temperature and stirred for 3 h. The reaction was concentrated under reduced pressure and dried under high vacuum for 30 min. to give an off-white solid. The crude solid was diluted in methylene chloride (10 mL) and cooled down to 0° C. A solution of 1-[(2-hydroxyethyl)sulfanyl]-3-methylbutan-1-one (93.1 mg, 574 μmol, 1 eq) in DCM (2 mL) (dried on Na₂SO₄) was added dropwise then triethylamine (299 μL, 2.87 mmol, 5 eq) in DCM (2 mL) (dried on Na₂SO₄) was slowly added on the yellow solution over 1 min. The reaction mixture was stirred at 0° C. for 1 min., then warmed up to room temperature and stirred for 1 h. The reaction was cooled down to 0° C. then benzyl (2R)-2-aminopropanoate 4-methylbenzene-1-sulfonic acid salt (302 mg, 861 μmol, 1.5 eq) was added in one portion. The reaction was warmed up to room temperature and stirred for 20 h. The reaction was concentrated under reduced pressure then diluted with DMSO/MeCN/water (3 mL). The crude residue was purified by reverse phase chromatography on a 100 g C₁₈ cartridge eluting with 5-100% MeCN in water to give allyl 5-(((((S)-1-(benzyloxy)-1-oxopropan-2-yl)amino)(2-((3-methylbutanoyl)thio)ethoxy)phosphoryl)difluoromethyl)benzo[b]thiophene-2-carboxylate (180 mg, 0.2753 mmol, 48.0% yield) as a thick brownish oil. ¹H NMR (400 MHz, CDCl₃) δ 8.16-8.12 (m, 1H), 7.94 (d, J=8.5 Hz, 0.5H), 7.78 (d, J=8.5 Hz, 0.5H), 7.70-7.64 (m, 1H), 7.43-7.31 (m, 6H), 6.12-6.01 (m, 1H), 5.47 (d, J=16.8 Hz, 1H), 5.35 (d, J=10.4 Hz, 1H), 5.25-5.11 (m, 2H), 4.90-4.86 (m, 2H), 4.24-4.00 (m, 2H), 3.82-3.69 (m, 1H), 3.24-2.92 (m, 2H), 2.45-2.40 (m, 2H), 2.18-2.10 (m, 1H), 1.44 (dd, J=16.0, 7.1 Hz, 3H), 0.98-0.94 (m, 6H).

Step 2: Preparation of 5-(((((S)-1-(benzyloxy)-1-oxopropan-2-yl)amino)(2-((3-methylbutanoyl)thio)ethoxy)phosphoryl)difluoromethyl)benzo[b]thiophene-2-carboxylic acid

To a stirred solution of allyl 5-(((((S)-1-(benzyloxy)-1-oxopropan-2-yl)amino)(2-((3-methylbutanoyl)thio)ethoxy)phosphoryl)difluoromethyl)benzo[b]thiophene-2-carboxylate (60 mg, 0.09178 mmol, 1 eq) in tetrahydrofuran (3 mL) were added morpholine (39.5 μL, 458 μmol, 5 eq) and tetrakis(triphenylphosphine)palladium (10.5 mg, 9.17 μmol, 0.10 eq) under nitrogen. The reaction mixture was stirred at room temperature for 1.5 h. The reaction was directly injected onto column. The product was purified by reverse phase chromatography on a 50 g C₁₈ cartridge eluting with 5-80% MeCN in water (containing 0.1% formic acid) and then freeze-dried to give 5-(((((S)-1-(benzyloxy)-1-oxopropan-2-yl)amino)(2-((3-methylbutanoyl)thio)ethoxy)phosphoryl)difluoromethyl)benzo[b]thiophene-2-carboxylic acid (40.0 mg, 0.06518 mmol, 71.1% yield) as an off-white sticky solid. LCMS: m/z=611.9 [M+H]⁺; ¹H NMR (400 MHz, CDCl₃) δ 8.10 (d, J=16.8 Hz, 1H), 7.86-7.78 (m, 2H), 7.61-7.54 (m, 1H), 7.45-7.35 (m, 5H), 5.25 (d, J=13.0 Hz, 2H), 4.60-4.45 (m, 1H), 4.40-4.14 (m, 3H), 3.36-3.14 (m, 2H), 2.48 (t, J=7.1 Hz, 3H), 2.25-2.13 (m, 1H), 1.58-1.51 (m, 3H), 1.02-0.96 (m, 6H).

### Synthesis of Building Blocks

Synthesis of 6-phenyl-4-azaspiro[2.4]heptane

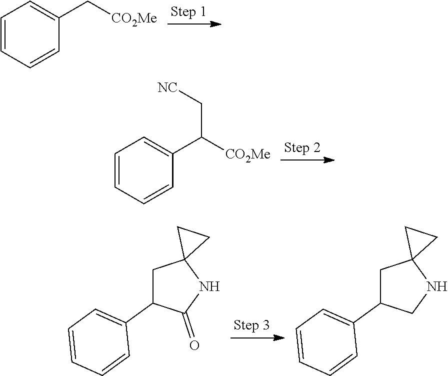

Step 1: Preparation of methyl 3-cyano-2-phenylpropanoate

To a cooled (−78° C.) solution of methyl 2-phenylacetate (5.0 g, 33.3 mmol, 1.0 eq) in dry THF (50 mL) was slowly added a solution of 2 M LDA (20 mL, 40.0 mmol, 1.2 eq) in THF. After 1 h, 2-bromoacetonitrile (4.2 g, 35.0 mmol, 1.1 eq) slowly in a dropwise manner and the reaction was further aged for additional 1 h at −78° C. To the mixture was added saturated aqueous NH₄Cl (5 mL) and the mixture was warmed to room temperature. The mixture was with EtOAc (20 mL×3). The organic layers were combined, washed with brine (150 mL), dried over anhydrous Na₂SO₄, and concentrated under reduced pressure. The residue was purified by flash column chromatography on silica gel to methyl 3-cyano-2-phenylpropanoate (5.0 g, 26.5 mmol, 79% yield) as a white solid. LCMS (ESI) m/z=190.1 [M+H]⁺.

Step 2: Preparation of 6-phenyl-4-azaspiro[2.4]heptan-5-one

To a cooled (0° C.) solution of methyl 3-cyano-2-phenylpropanoate (5.0 g, 26.5 mmol, 1.0 eq) and Ti(OiPr)₄ (9.0 g, 31.7 mmol, 1.2 eq) in dry THF (100 mL) was slowly added a 3 M solution of EtMgBr (20 mL, 59.6 mmol, 2.25 eq), while maintaining inner temperature between −5° C. to 0° C. After complete addition of EtMgBr, the mixture was stirred at 0° C. for additional 1 h. The reaction mixture was quenched by addition of aqueous 2 N HCl (60 mL). The resulting acidic solution was extracted with EtOAc (50 mL×3). The combined organic layers were washed with brine (20 mL×2), dried over anhydrous Na₂SO₄, and concentrated under reduced pressure. The residue was purified by flash column chromatography on to afford 6-phenyl-4-azaspiro[2.4]heptan-5-one (2.1 g, 11.2 mmol, 42% yield) as a white solid. LCMS (ESI) m/z=188 [M+H]⁺; ¹H NMR (400 MHz, CDCl₃) δ 7.39-7.24 (m, 6H), 3.86 (dd, J=9.4, 7.6 Hz, 1H), 2.51 (dd, J=12.9, 9.4 Hz, 1H), 2.29 (dd, J=12.9, 7.6 Hz, 1H), 0.85-0.88 (m, 1H), 0.87-0.81 (m, 1H), 0.76-0.64 (m, 2H).

Step 3: Preparation of 6-phenyl-4-azaspiro[2.4]heptane

To a cooled (10° C.) solution of 6-phenyl-4-azaspiro[2.4]heptan-5-one (2.1 g, 11.2 mmol, 1.0 eq) in dry THF (50 mL) was added NaBH₄ (2.1 g, 56.0 mmol, 5.0 eq) in several portions. To the mixture was added in a dropwise manner BF₃·Et₂O (6.7 mL, 56.0 mmol, 5.0 eq). The reaction mixture was heated at 60° C. for 16 h. The mixture was cooled to ambient temperature and aqueous 2 N HCl (30 mL) was introduced slowly. The acidic mixture was extracted with EtOAc (30 mL×3). The organic layers were combined, washed with brine (20 mL), dried over anhydrous Na₂SO₄, and concentrated under reduced pressure. The residue was purified by Biotage® C18 column to afford 6-phenyl-4-azaspiro[2.4]heptane (1.5 g, 8.7 mmol, 78% yield) as a colorless oil. LCMS (ESI) m/z=174 [M+H]⁺; ¹H NMR (400 MHz, CDCl₃) 8.59 (s, 1H), 6 7.38-7.27 (m, 5H), 3.85-3.57 (m, 2H), 3.40-3.17 (m, 1H), 2.43-2.09 (m, 2H), 1.46-1.18 (m, 2H), 0.98-0.66 (m, 2H).

### Pyrrolidine Building Block Synthesis:

Synthesis of rel-(trans)-4-cyclohexylpyrrolidine-3-carbonitrile

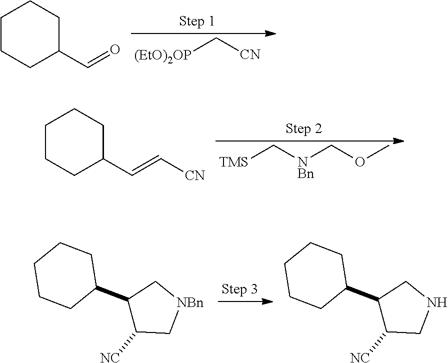

Step 1: Preparation of (E)-3-cyclohexylacrylonitrile

To a solution of cyclohexanecarbaldehyde (500 mg, 4.45 mmol, 1.0 eq) in THF (15 mL) were added t-BuOK (998 mg, 8.90 mmol, 2.0 eq) and diethyl (cyanomethyl)phosphonate (788 mg, 4.45 mmol, 1.0 eq) at room temperature. The solution was stirred at room temperature for 1 h. After completion, the reaction mixture was quenched by adding H₂O (10 mL), then extracted with EtOAc (10 mL×3). The organic layers were combined and washed with brine (10 mL), dried over anhydrous Na₂SO₄, filtered, and concentrated under reduced pressure. The residue was purified by flash column chromatography on silica gel to afford (E)-3-cyclohexylacrylonitrile (300 mg, 2.21 mmol, 50% yield) as a colorless oil. LC-MS (ESI) m/z=136 [M+H]⁺.

Step 2: Preparation of rel-(trans)-1-benzyl-4-cyclohexylpyrrolidine-3-carbonitrile

To a solution of (E)-3-cyclohexylacrylonitrile (300 mg, 2.21 mmol, 1.0 eq) in CH₂Cl₂ (5 mL) were added N-benzyl-1-methoxy-N-((trimethylsilyl)methyl)methanamine (524 mg, 2.21 mmol, 1.0 eq) and TFA (25.1 mg, 221 μmol, 0.1 eq). The reaction mixture was stirred at room temperature for 12 h. After completion, the reaction mixture was dilute with CH₂Cl₂ (10 mL) and washed with aqueous saturated NaHCO₃ (5 mL), the organic layer was separated and concentrated under reduced pressure to afford crude rel-(trans)-1-benzyl-4-cyclohexylpyrrolidine-3-carbonitrile (700 mg) as a colorless oil. LC-MS (ESI) m/z=269 [M+H]⁺.

Step 3: Preparation of rel-(trans)-4-cyclohexylpyrrolidine-3-carbonitrile

To a solution of (trans)-1-benzyl-4-cyclohexylpyrrolidine-3-carbonitrile (700 mg) in dry 1,2-dichloroethane (15 mL) was added 1-chloroethyl carbonochloridate (3.71 g, 26.0 mmol, 10.0 eq). The resulting mixture was stirred at 70° C. for 12 h, then concentrated under reduced pressure, the crude product was dissolved in MeOH (5 mL) and stirred at 70° C. for 1 hr. After completion, the reaction solution was cooled to room temperature and concentrated under reduced pressure. The residue was purified by flash column chromatography on silica gel to afford rel-(trans)-4-cyclohexylpyrrolidine-3-carbonitrile (211 mg, 1.18 mmol, 46% yield) as a colorless oil. LC-MS (ESI) m/z=179 [M+H]⁺.

The following intermediates in Table 18 were prepared according to the representative procedures (Step 1 through Step 3) described for rel-(trans)-4-cyclohexylpyrrolidine-3-carbonitrile utilizing appropriate starting materials and modifications. Compounds were prepared as racemates with trans stereochemical configuration relative to the C3 and C4 stereocenters within the pyrrolidine ring.

TABLE 18

| Name | Structure | LCMS |
|---|---|---|
| rel-(trans)-4- (2-oxo-1,2-dihydropyridin- 4-yl)pyrrolidine-3- carbonitrile | 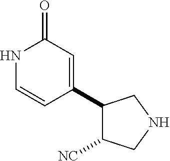 | 190 [M + H]⁺ |
| rel-(trans)-3-methyl-4- phenylpyrrolidine-3- carbonitrile | 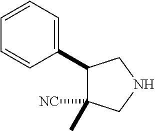 | 187.2 [M + H]⁺ |

tert-Butyl rel-(trans)-3-cyano-4-phenylpyrrolidine-1-carboxylate and tert-butyl (trans)-3-cyano-4-(2-oxo-1,2-dihydropyridin-4-yl)pyrrolidine-1-carboxylate were prepared according to the method describe above for the synthesis of rel-(trans)-4-cyclohexylpyrrolidine-3-carbonitrile. The racemic mixture of trans-isomers were purified under SFC conditions and the absolute stereochemistry was arbitrarily assigned as drawn.

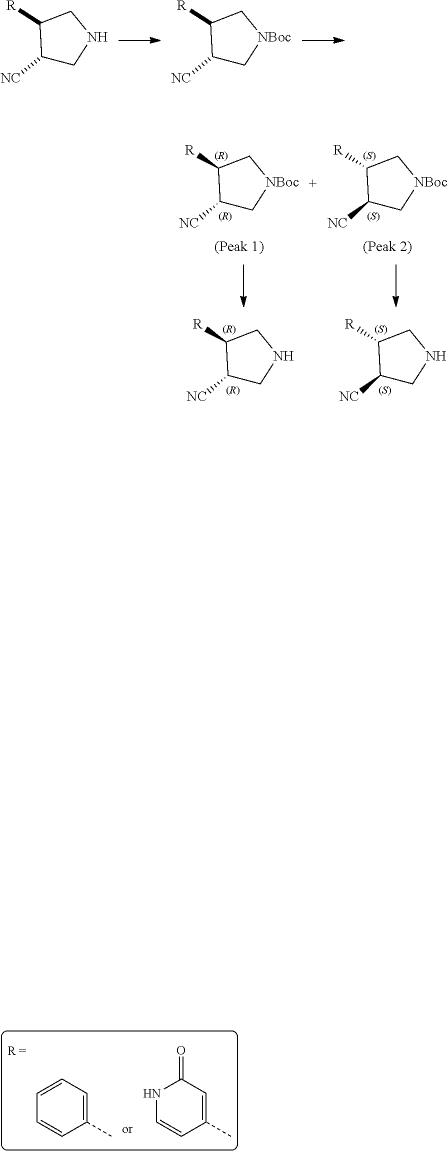

### Preparative Separation Method:

Instrument: Waters Thar 80 preparative SFC; Column: ChiralPak C-IG, 100×4.6 mm I.D., 5 μm; Mobile phase: A for CO₂ and B for methanol (0.05% diethylamine); Gradient: 10% to 40% B in 8 min; Flow rate: 2.5 mL/min; Back pressure: 100 bar; Column temperature: 40° C.; Wavelength: 210 nm; Cycle-time: 2 min

The following intermediates in Table 19 were prepared according to the procedure described for rel-(trans)-4-cyclohexylpyrrolidine-3-carbonitrile utilizing appropriate starting materials and modifications.

TABLE 19

| Name | Structure | LCMS |
|---|---|---|
| tert-butyl rel- (trans)-3-cyano-4- phenylpyrrolidine- 1-carboxylate (Peak 1) | 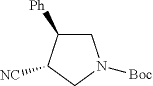 [SMILES: CC(C)(C)OC(=O)N1C[C@H](c2ccccc2)[C@@H](C#N)C1] | 217 [(M − ^{t}Bu) + 1]⁺ |
| tert-butyl rel- (trans)-3-cyano-4- phenylpyrrolidine- 1-carboxylate (Peak 2) | 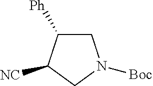 [SMILES: CC(C)(C)OC(=O)N1C[C@@H](C#N)[C@H](c2ccccc2)C1] | 217 [(M − ^{t}Bu) + 1]⁺ |
| tert-butyl rel- (trans)-3- cyano-4-(2-oxo-1,2- dihydropyridin-4- yl)pyrrolidine-1- carboxylate (Peak 1) | 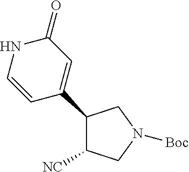 [SMILES: CC(C)(C)OC(=O)N1C[C@H](c2cc[nH]c(=O)c2)[C@@H](C#N)C1] | 190 [M + H]⁺ |
| tert-butyl rel- (trans)-3-cyano- 4-(2-oxo-1,2- dihydropyridin-4- yl)pyrrolidine-1- carboxylate (Peak 2) | 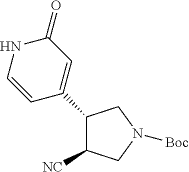 [SMILES: CC(C)(C)OC(=O)N1C[C@@H](C#N)[C@H](c2cc[nH]c(=O)c2)C1] | 190 [M + H]⁺ |

Synthesis of 2-(4-amino-[1,1′-biphenyl]-3-yl)acetamide

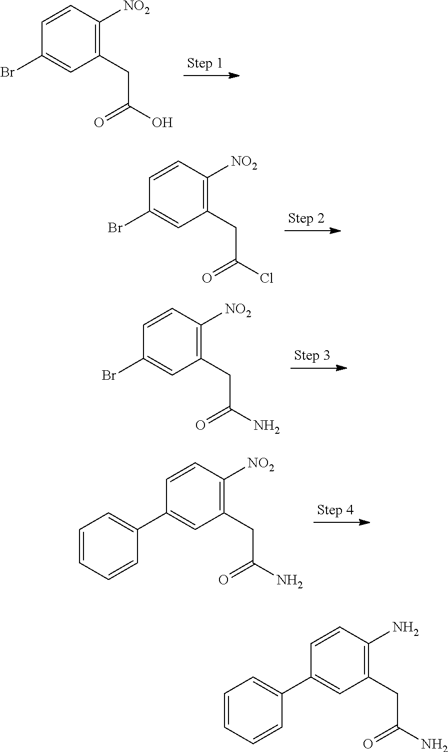

Step 1: Preparation of 2-(5-bromo-2-nitrophenyl)acetyl chloride

A solution of 2-(5-bromo-2-nitrophenyl)acetic acid (0.30 g, 1.2 mmol, 1 eq) in thionyl chloride (2 mL) was stirred at 80° C. for 1 h. The reaction mixture was concentrated under reduced pressure to give 2-(5-bromo-2-nitrophenyl)acetyl chloride (0.30 g, crude) as a white solid.

Step 2: Preparation of 2-(5-bromo-2-nitrophenyl)acetamide

A solution of 2-(5-bromo-2-nitrophenyl)acetyl chloride (0.30 g, 1.1 mmol, 1 eq) in tetrahydrofuran (3 mL) and NH₃—H₂O (4 mL) was stirred at 25° C. for 30 min. The reaction mixture was concentrated under reduced pressure to give 2-(5-bromo-2-nitrophenyl)acetamide (0.30 g, crude) as a white solid. LCMS: m/z [M+H]⁺=258.9.

Step 3: Preparation of 2-(4-nitro-[1,1′-biphenyl]-3-yl)acetamide

To a solution of 2-(5-bromo-2-nitrophenyl)acetamide (0.10 g, 0.39 mmol, 1 eq) in dioxane (1 mL) and water (0.2 mL) was added sodium carbonate (0.12 g, 1.2 mmol, 3 eq), phenylboronic acid (52 mg, 0.42 mmol, 1.1 eq) iron(2+) bis(cyclopenta-2,4-diyn-1-yldiphenyl-lambda4-phosphane) palladium dichloride (14 mg, 0.019 mmol, 0.05 eq), the mixture was stirred at 100° C. for 12 h. The mixture was diluted with water (20 mL) and extracted with EtOAc (20 mL×2), the combined organic layers were washed with saturated brine (20 mL×2), dried over Na₂SO₄, filtered, and concentrated under reduced pressure to give a residue. The residue was purified by column chromatography to give 2-(4-nitro-[1,1′-biphenyl]-3-yl)acetamide (60 mg, 60% yield) as a white solid. LCMS: m/z (M+Na)⁺=279.0. ¹H NMR (400 MHz, CD₃OD) δ 8.17 (d, J=8.4 Hz, 1H), 7.79-7.69 (m, 4H), 7.52-7.40 (m, 3H), 4.06 (s, 2H)

Step 4: Preparation of 2-(4-amino-[1,1′-biphenyl]-3-yl)acetamide

To a solution of 2-(4-nitro-[1,1′-biphenyl]-3-yl)acetamide (60 mg, 0.23 mmol, 1 eq) in ethyl alcohol (0.8 mL) and water (0.2 mL) was added ammonium chloride (0.038 g, 0.70 mmol, 3 eq) and iron (0.13 g, 2.4 mmol, 10 eq). The mixture was stirred at 25° C. for 4 h. The reaction mixture was filtered and the filtrate was concentrated to give 2-(4-amino-[1,1′-biphenyl]-3-yl)acetamide (0.043 g, crude) as a white solid. LCMS: m/z [M+H]⁺=227.0. ¹H NMR (400 MHz, DMSO-d₆) δ 7.53 (d, J=7.6 Hz, 3H), 7.41-7.35 (m, 3H), 7.30-7.20 (m, 2H), 6.98 (s, 1H), 6.73 (d, J=8.4 Hz, 1H), 5.26 (s, 2H), 3.32 (s, 2H).

Synthesis of 1-((3S,4R)-3-amino-4-fluoropyrrolidin-1-yl)ethan-1-one

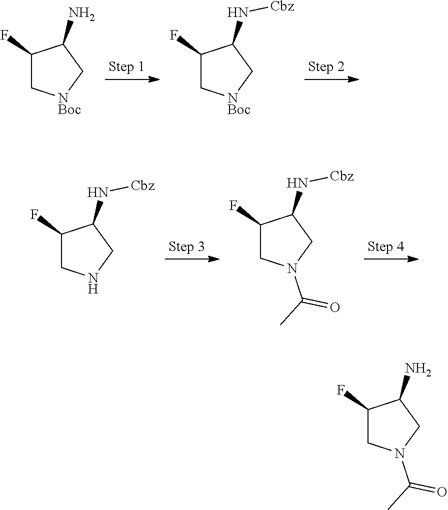

Step 1: Preparation of tert-butyl (3S,4R)-3-(((benzyloxy)carbonyl)amino)-4-fluoropyrrolidine-1-carboxylate

To a solution of tert-butyl (3S,4R)-3-amino-4-fluoropyrrolidine-1-carboxylate (250 mg, 1.22 mmol, 1.0 eq.) and sodium hydrogen carbonate (512 mg, 6.10 mmol, 5.0 eq.) in THF (10 mL) and water (2 mL) was added benzyl carbonochloridate (416 mg, 2.44 mmol, 2.0 eq.) at room temperature. After addition, the reaction mixture was stirred at room temperature overnight. After completion, the reaction mixture was poured into water (10 mL) and extracted with EtOAc (10 mL×3). The combined organic phase was washed with brine (10 mL), dried over anhydrous Na₂SO₄, and concentrated under reduced pressure to afford tert-butyl (3S,4R)-3-(((benzyloxy)carbonyl)amino)-4-fluoropyrrolidine-1-carboxylate (2, 400 mg, 1.18 mmol, 97%) as a yellow oil, which was used in next step directly without further purification. LCMS (ESI): m/z=361.1 [M+Na]⁺.

Step 2: Preparation of benzyl ((3S,4R)-4-fluoropyrrolidin-3-yl)carbamate

A solution of tert-butyl (3S,4R)-3-(((benzyloxy)carbonyl)amino)-4-fluoropyrrolidine-1-carboxylate (200 mg, 0.59 mmol, 1.0 eq.) in DCM (1 mL) and TFA (0.5 mL) was stirred at 25° C. overnight under N₂. After completion, the reaction mixture was concentrated under reduced pressure to get crude benzyl ((3S,4R)-4-fluoropyrrolidin-3-yl)carbamate (140 mg, quant.) as a white solid, which was used in next step directly without further purification. LCMS (ESI): m/z=239.3 [M+H]⁺.

Step 3: Preparation of benzyl ((3S,4R)-1-acetyl-4-fluoropyrrolidin-3-yl)carbamate

To a solution of benzyl ((3S,4R)-4-fluoropyrrolidin-3-yl)carbamate (140 mg, 0.59 mmol, 1.0 eq.) and TEA (179 mg, 1.77 mmol, 3.0 eq.) in dry DCM (6 mL) was added acetyl chloride (46.0 mg, 0.59 mmol, 1.0 eq.) at 0° C. After addition, the resulting mixture was warmed up to 20° C. and stirred for 2 h under N₂. After completion, the reaction mixture was poured into water (10 mL) and extracted with DCM (10 mL×3). The organic layers were combined and washed with brine (10 mL), dried over anhydrous Na₂SO₄, and concentrated under reduced pressure. The residue was purified by flash column chromatography on silica gel to afford benzyl ((3S,4R)-1-acetyl-4-fluoropyrrolidin-3-yl)carbamate (611 mg, 1.29 mmol, 62%) as a white solid. LCMS (ESI): m/z=281.1 [M+H]⁺.

Step 4: Preparation of 1-((3S,4R)-3-amino-4-fluoropyrrolidin-1-yl)ethan-1-one

To a solution of benzyl ((3S,4R)-1-acetyl-4-fluoropyrrolidin-3-yl)carbamate (250 mg, 1.22 mmol, 1.0 eq.) in EtOH (10 mL) was added Pd/C (50 mg) under nitrogen. The suspension was degassed under vacuum and purged with H₂ several times. The resulting mixture was stirred at room temperature overnight. After completion, the suspension was filtered through a pad of Celite®, the filter cake was washed with EtOH (20 mL). The combined filtrates were concentrated to dryness to give 1-((3S,4R)-3-amino-4-fluoropyrrolidin-1-yl)ethan-1-one (375 mg, 2.10 mmol, 87%) as a colorless oil. LCMS (ESI): m/z=147.1 [M+H]⁺.

Synthesis of (R)-azetidin-3-yl(2-methylmorpholino)methanone

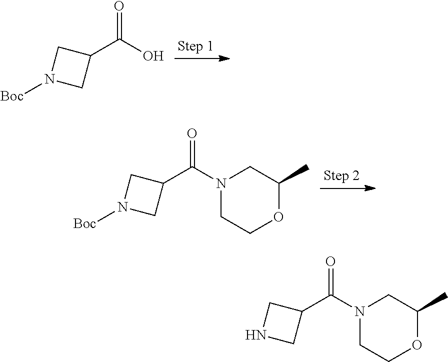 [SMILES: CC(C)(C)OC(=O)N1CC(C(=O)O)C1 ; C[C@@H]1CN(C(=O)C2CN(C(=O)OC(C)(C)C)C2)CCO1 ; C[C@@H]1CN(C(=O)C2CNC2)CCO1]

Step 1: Preparation of tert-butyl (R)-3-(2-methylmorpholine-4-carbonyl)azetidine-1-carboxylate

To a solution of 1-[(tert-butoxy)carbonyl]azetidine-3-carboxylic acid (1, 100 mg, 496 μmol, 1.0 eq.), HATU (226 mg, 595 μmol, 1.2 eq.), and triethylamine (100 mg, 992 μmol, 2.0 eq.) in DMF (2 mL) was added (2R)-2-methylmorpholine (55.1 mg, 545 μmol, 1.1 eq.) and the resulting mixture was stirred at 20° C. overnight under N₂. After completion, the reaction mixture was filtered and concentrated under reduced pressure. The residue was purified by Biotage® C18 column chromatography to get tert-butyl (R)-3-(2-methylmorpholine-4-carbonyl)azetidine-1-carboxylate (2, 140 mg, 492 μmol, 99%) as a white solid. LCMS (ESI): m/z=229.2 [(M−56)+H]⁺.

Step 2: Preparation of (R)-azetidin-3-yl(2-methylmorpholino)methanone

A solution of tert-butyl (R)-3-(2-methylmorpholine-4-carbonyl)azetidine-1-carboxylate (2, 50 mg, 175 μmol, 1.0 eq.) in DCM (6 mL) and TFA (2 mL) was stirred at 25° C. overnight under N₂. After completion, the reaction mixture was concentrated to get crude (R)-azetidin-3-yl(2-methylmorpholino)methanone (3, 40.0 mg, quant.) as a white solid, which was used in next step directly without further purification. LCMS (ESI): m/z=185.3 [M+H]⁺.

The following building blocks were prepared according to the above scheme:

The following intermediates in Table 20 were prepared according to the procedures described for (R)-azetidin-3-yl(2-methylmorpholino)methanone utilizing appropriate starting materials and modifications.

TABLE 20

| Name | Structure | LCMS |
|---|---|---|
| (R)-azetidin-3-yl(3- methylmorpholino) methanone | 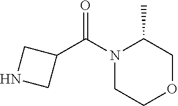 | 185 [M + H]⁺ |
| (S)-azetidin-3-yl(2- methylmorpholino) methanone | 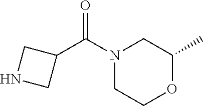 | 185.2 [M + H]⁺ |
| (S)-azetidin-3-yl(3- methylmorpholino) methanone | 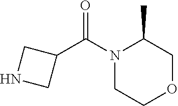 | 185 [M + H]⁺ |

Synthesis of (R)-morpholino(pyrrolidin-3-yl)methanone

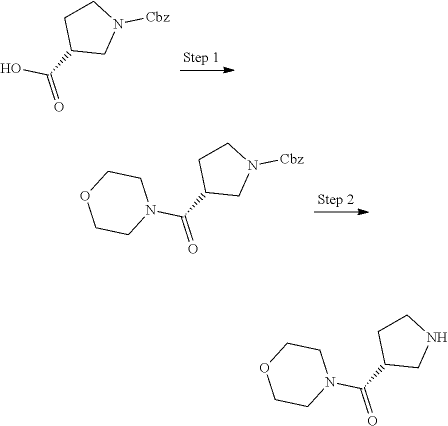

Step 1: Preparation of benzyl (R)-3-(morpholine-4-carbonyl)pyrrolidine-1-carboxylate

To a solution of (R)-1-((benzyloxy)carbonyl)pyrrolidine-3-carboxylic acid (0.15 g, 0.60 mmol) and HATU (0.34 g, 0.90 mmol) in DMF (1 mL) was added morpholine (58 mg, 0.66 mmol) and DIEA (0.23 g, 1.8 mmol) and stirred at 15° C. for 1 h to give a brown solution. The solution was quenched by water (10 mL) and extracted with EtOAc (10 mL×2), the combined organic layers were washed with saturated brine (10 mL×3), dried over Na₂SO₄, filtered, and concentrated under reduced pressure. The residue was purified by silica gel chromatography to give benzyl (R)-3-(morpholine-4-carbonyl)pyrrolidine-1-carboxylate (0.10 g, 52% yield) as a brown oil. ¹H NMR (400 MHz, CDCl₃) δ 7.41-7.28 (m, 5H), 5.14 (s, 2H), 3.68 (d, J=4.4 Hz, 9H), 3.53-3.40 (m, 3H), 3.24-3.16 (m, 1H), 2.34-2.13 (m, 1H), 1.49-1.42 (m, 1H).

Step 2: Preparation of (R)-morpholino(pyrrolidin-3-yl)methanone

To a solution of benzyl (R)-3-(morpholine-4-carbonyl)pyrrolidine-1-carboxylate (0.10 g, 0.31 mmol) in MeOH (5 mL) was added wet Pd/C (0.10 g) and stirred at 15° C. under H₂ (15 psi) for 2 h to give a black suspension. The reaction mixture was filtered and concentrated in vacuum to give (R)-morpholino (pyrrolidin-3-yl)methanone (60 mg, crude) as a colorless oil. LCMS: (ESI) m/z [M+H]⁺=185.3

The following intermediates in Table 21 were prepared according to the procedures described for (R)-morpholino(pyrrolidin-3-yl)methanone utilizing appropriate starting materials and modifications.

TABLE 21

| Name | Structure | LCMS |
|---|---|---|
| (S)-morpholino(pyrrolidin- 3-yl)methanone | 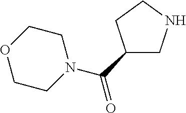 [SMILES: O=C([C@H]1CCNC1)N1CCOCC1] | 185 [M + H]⁺ |

Synthesis of (S)-1-(tetrahydrofuran-3-yl)azetidin-3-amine

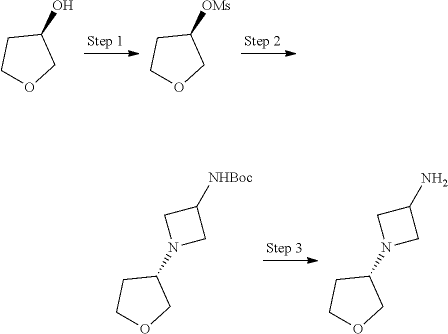

Step 1: Preparation of (R)-tetrahydrofuran-3-yl methanesulfonate

To a solution of (R)-tetrahydrofuran-3-ol (2.0 g, 23 mmol, 1.0 eq) and TEA (6.9 g, 69 mmol, 3.0 eq) in DCM (20 mL) was added dropwise methanesulfonyl chloride (3.4 g, 29 mmol, 1.3 eq) at 0° C., the mixture was stirred at 25° C. for 2 h to give a yellow mixture. The mixture was quenched by H₂O (50 mL) and extracted with DCM (50 mL×2), the combined organic layers were dried over Na₂SO₄, filtered, and concentrated to give (R)-tetrahydrofuran-3-yl methanesulfonate (3.5 g, crude) as a yellow oil. ¹H NMR (400 MHz, CDCl₃) δ 5.28-5.20 (m, 1H), 3.99-3.75 (m, 4H), 2.97 (s, 3H), 2.23-2.11 (m, 2H)

Step 2: Preparation of tert-butyl (S)-(1-(tetrahydrofuran-3-yl)azetidin-3-yl)carbamate

To a solution of (R)-tetrahydrofuran-3-yl methanesulfonate (1.5 g, 9.0 mmol, 1.0 eq) and tert-butyl azetidin-3-ylcarbamate (4.7 g, 27 mmol, 3.0 eq) in MeCN (15 mL) was added K₂CO₃ (5.6 g, 41 mmol, 4.5 eq), the mixture was stirred at 80° C. for 12 h to give a light-yellow suspension. The mixture was filtered and concentrated to give a residue. The residue was purified by column chromatography to give tert-butyl (S)-(1-(tetrahydrofuran-3-yl)azetidin-3-yl)carbamate (0.6 g, crude) was obtained as a yellow oil. LCMS: (ESI) m/z [M+H]⁺=243.0

Step 3: Preparation of (S)-1-(tetrahydrofuran-3-yl)azetidin-3-amine

A solution of tert-butyl (S)-(1-(tetrahydrofuran-3-yl)azetidin-3-yl)carbamate (0.10 mg, 0.41 mmol, 1 eq) in DCM (1.5 mL) and TFA (0.50 mL) was stirred at 25° C. for 2 h to give a pink solution. The reaction mixture was concentrated under reduced pressure to give (S)-1-(tetrahydrofuran-3-yl)azetidin-3-amine (0.10 mg, crude) as a yellow oil. LCMS: (ESI) m/z [M+H]⁺=143.3

The following intermediates in Table 22 were prepared according to the procedures described for (S)-1-(tetrahydrofuran-3-yl)azetidin-3-amine utilizing appropriate starting materials and modifications.

TABLE 22

| Name | Structure | LCMS |
|---|---|---|
| (R)-1-(tetrahydrofuran- 3-yl)azetidin-3-amine | 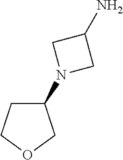 | 143 [M + H]⁺ |

Synthesis of (3S,4R)-4-fluoro-1-(pyridin-3-yl)pyrrolidin-3-amine

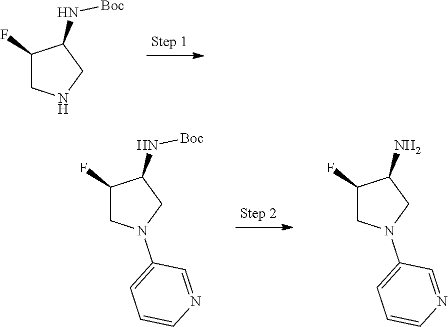 [SMILES: CC(C)(C)OC(=O)N[C@H]1CNC[C@H]1F ; CC(C)(C)OC(=O)N[C@H]1CN(c2cccnc2)C[C@H]1F ; N[C@H]1CN(c2cccnc2)C[C@H]1F]

Step 1: Preparation of tert-butyl ((3S,4R)-4-fluoro-1-(pyridin-3-yl)pyrrolidin-3-yl)carbamate

To a solution of tert-butyl ((3S,4R)-4-fluoropyrrolidin-3-yl)carbamate (0.1 g, 0.49 mmol, 1.0 eq) in dioxane (2.5 mL) was added 3-bromopyridine (93 mg, 0.59 mmol, 1.2 eq), Xantphos (25 mg, 44 mol, 0.1 eq) and cesium carbonate (0.25 g, 0.78 mmol, 1.6 eq), the reaction was degassed and purged with N₂ three times, then was added Pd₂(dba)₃ (0.13 g, 14 mol, 0.3 eq). The resulting reaction mixture was stirred at 100° C. for 72 h to give a black suspension. The reaction mixture was filtered and the filtrate was concentrated to give a residue. The residue was diluted with water (50 mL) and extracted with EtOAc (50 mL×2), and the combined organic layers were washed with saturated brine (50 mL×2), dried over Na₂SO₄, filtered, and concentrated under reduced pressure to give a residue. The residue was purified by flash silica gel chromatography to give tert-butyl ((3S,4R)-4-fluoro-1-(pyridin-3-yl)pyrrolidin-3-yl)carbamate (0.1 g, 73% yield) as a yellow oil. LCMS: (ESI) m/z [M+H]⁺=282.1

Step 2: Preparation of (3S,4R)-4-fluoro-1-(pyridin-3-yl)pyrrolidin-3-amine

A solution of tert-butyl ((3S,4R)-4-fluoro-1-(pyridin-3-yl)pyrrolidin-3-yl)carbamate (0.1 g, 0.36 mmol, 1.0 eq) in DCM (0.3 mL) and trifluoroacetic acid (0.1 mL) was stirred at 20° C. for 1 h to give a yellow solution. The reaction mixture was concentrated under reduced pressure to give (3S,4R)-4-fluoro-1-(pyridin-3-yl)pyrrolidin-3-amine (0.1 g, crude) as a yellow oil. LCMS: (ESI) m/z [M+H]⁺=182.1

The following intermediates in Table 23 were prepared according to the procedures described for (3S,4R)-4-fluoro-1-(pyridin-3-yl)pyrrolidin-3-amine utilizing appropriate starting materials and modifications.

TABLE 23

| Name | Structure | LCMS |
|---|---|---|
| (3S,4S)-4-fluoro-1- (pyridin-3- yl)pyrrolidin-3-amine | 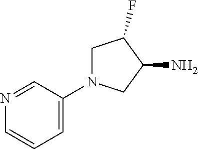 [SMILES: N[C@H]1CN(c2cccnc2)C[C@@H]1F] | 182 [M + H]⁺ |

Preparation of 1-((3S,4S)-3-amino-4-fluoropyrrolidin-1-yl)ethan-1-one

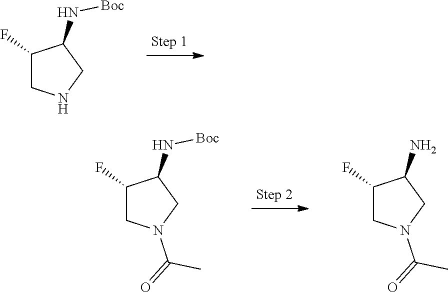

Step 1: Preparation of tert-butyl ((3S,4S)-1-acetyl-4-fluoropyrrolidin-3-yl)carbamate

A solution of tert-butyl ((3S,4S)-4-fluoropyrrolidin-3-yl)carbamate (0.1 g, 0.49 mmol, 1 eq) in DCM (0.5 mL) was added TEA (0.1 g, 0.98 mmol, 2 eq), the mixture was stirred at 0° C. for 5 minutes, then was added a solution of Ac₂O (0.05 g, 0.49 mmol, 1 eq) in DCM (0.5 mL), the mixture was stirred at 25° C. for 30 minutes. The reaction mixture was diluted with water (50 mL) and extracted with EtOAc (50 mL×2), the combined organic layers were washed with saturated brine (50 mL×2), dried over Na₂SO₄, filtered, and concentrated under reduced pressure to give a residue. The residue was purified by column chromatography (SiO₂, Petroleum ether/EtOAc=10:1 to 3:1) to give tert-butyl ((3S,4S)-1-acetyl-4-fluoropyrrolidin-3-yl)carbamate (60 mg, 49% yield) as a yellow oil. LCMS: (ESI) m/z [M+H]⁺=247.1

Step 2: Preparation of 1-((3S,4S)-3-amino-4-fluoropyrrolidin-1-yl)ethanone

A solution of tert-butyl ((3S,4S)-1-acetyl-4-fluoropyrrolidin-3-yl)carbamate (60 mg, 0.24 mmol, 1 eq) in TFA (0.3 mL) and DCM (0.9 mL) was stirred at 25° C. for 30 minutes to give yellow solution. The reaction mixture was concentrated under reduced pressure to give 1-((3S,4S)-3-amino-4-fluoropyrrolidin-1-yl)ethanone (60 mg, crude) as a yellow oil. LCMS: (ESI) m/z [M+H]⁺=147.2

The following intermediates in Table 24 were prepared according to the procedures described for 1-((3S,4S)-3-amino-4-fluoropyrrolidin-1-yl)ethan-1-one utilizing appropriate starting materials and modifications.

TABLE 24

| Name | Structure | LCMS |
|---|---|---|
| 1-((3S,4R)-3-amino-4- fluoropyrrolidin-1-yl)ethan- 1-one | 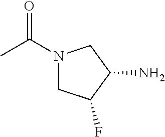 | 147 [M + H]⁺ |

Synthesis of 2-phenyl-1-(2,6-diazaspiro[3.3]heptan-2-yl)ethan-1-one

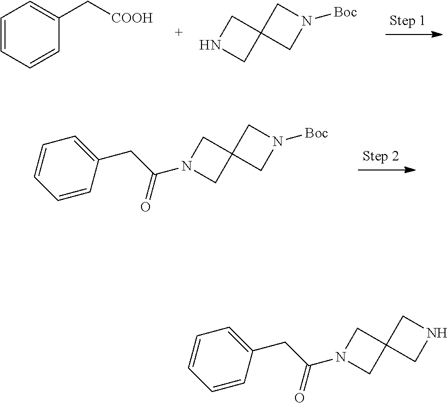 [SMILES: O=C(O)Cc1ccccc1 ; CC(C)(C)OC(=O)N1CC2(CNC2)C1 ; CC(C)(C)OC(=O)N1CC2(CN(C(=O)Cc3ccccc3)C2)C1 ; O=C(Cc1ccccc1)N1CC2(CNC2)C1]

Step 1: Preparation of tert-butyl 6-(2-phenylacetyl)-2,6-diazaspiro[3.3]heptane-2-carboxylate

To a solution of 2-phenylacetic acid (0.20 g, 1.5 mmol, 1 eq) in DMF (1 mL) was added HATU (0.83 g, 2.2 mmol, 1.5 eq) and DIEA (0.38 g, 3.0 mmol, 2 eq), then the solution of tert-butyl 2,6-diazaspiro [3.3]heptane-2-carboxylate (0.35 g, 1.8 mmol, 1.2 eq) in DMF (1 mL) and DIEA (0.57 g, 4.5 mmol, 3 eq) was added, the mixture was stirred at 25° C. for 2 h to give a yellow solution. The mixture was quenched by water (10 mL) and extracted with EtOAc (10 mL×2), the combined organic layers were washed with saturated brine (10 mL×2), dried over Na₂SO₄, filtered, and concentrated under reduced pressure to give a residue. The residue was purified by column chromatography (SiO₂, DCM:MeOH=100:1 to 10:1) to give tert-butyl 6-(2-phenylacetyl)-2,6-diazaspiro[3.3]heptane-2-carboxylate (0.100 g, 23% yield) as a yellow oil. LCMS: (ESI) m/z [M+H]⁺=317.0

Step 2: Preparation of 2-phenyl-1-(2,6-diazaspiro[3.3]heptan-2-yl)ethanone

To a solution of tert-butyl 6-(2-phenylacetyl)-2,6-diazaspiro[3.3]heptane-2-carboxylate (0.1 g, 0.32 mmol, 1 eq) in DCM (3 mL) and TFA (1 mL), the mixture was stirred at 25° C. for 2 h to give a yellow solution. The reaction mixture was concentrated under reduced pressure to give 2-phenyl-1-(2,6-diazaspiro[3.3]heptan-2-yl)ethanone (0.100 g, crude) as a yellow oil (3-methylazetidin-3-yl)(piperidin-1-yl)methanone: ¹H NMR (400 MHz, CDCl₃) δ 4.07-3.96 (m, 2H), 3.67 (s, 2H), 3.24-3.13 (m, 2H), 3.09 (s, 2H), 2.99 (s, 2H), 2.91 (s, 2H), 1.74 (s, 3H).

The following intermediates in Table 25 were prepared according to the procedures described for 2-phenyl-1-(2,6-diazaspiro[3.3]heptan-2-yl)ethan-1-one utilizing appropriate starting materials and modifications.

TABLE 25

| Name | Structure | LCMS |
|---|---|---|
| (3-fluoroazetidin-3- yl)(morpholino) methanone | 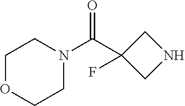 [SMILES: O=C(N1CCOCC1)C1(F)CNC1] | 189.2 [M + H]⁺ |
| azetidin-3-yl(3- methoxyazetidin- 1-yl)methanone | 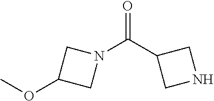 | 171.0 [M + H]⁺ |
| 1-(3-aminoazetidin- 1-yl)-4,4,4- trifluorobutan-1-one | 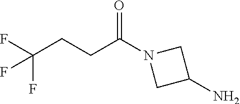 [SMILES: NC1CN(C(=O)CCC(F)(F)F)C1] | 197.0 [M + H]⁺ |

Synthesis of 4-(3-fluoroazetidin-3-yl)benzonitrile

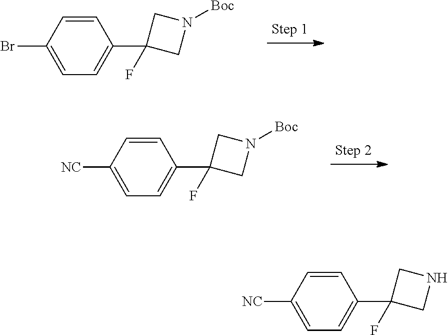 [SMILES: CC(C)(C)OC(=O)N1CC(F)(c2ccc(Br)cc2)C1 ; CC(C)(C)OC(=O)N1CC(F)(c2ccc(C#N)cc2)C1 ; N#Cc1ccc(C2(F)CNC2)cc1]

Step 1: Preparation of tert-butyl 3-(4-cyanophenyl)-3-fluoroazetidine-1-carboxylate

To a solution of tert-butyl 3-(4-bromophenyl)-3-fluoroazetidine-1-carboxylate (0.50 g, 1.5 mmol, 1.0 eq) in DMF (10 mL) was added K₄Fe(CN)₆ (0.16 g, 0.38 mmol, 0.25 eq), sodium carbonate (0.16 mg, 1.51 mmol, 1.0 eq) and Pd(dppf)Cl₂ (55 mg, 75.5 mol, 0.05 eq), the mixture was stirred at 25° C. for 12 h to give a black mixture. The reaction mixture was filtered and the filtrate was diluted with water (50 mL) and extracted with EtOAc (50 mL×2), the combined organic layers were washed with saturated brine (50 mL×2), dried over Na₂SO₄, filtered, and concentrated under reduced pressure to give a residue. The residue was purified by flash silica gel chromatography to give tert-butyl 3-(4-cyanophenyl)-3-fluoroazetidine-1-carboxylate (0.35 g, 83% yield) as a white solid. LCMS: (ESI) m/z [M+H]⁺=277.0

Step 2: Preparation of 4-(3-fluoroazetidin-3-yl)benzonitrile

A solution of tert-butyl 3-(4-cyanophenyl)-3-fluoroazetidine-1-carboxylate (0.25 mg, 0.90 mmol, 1.0 eq) in DCM (1.5 mL) and TFA (0.50 mL) was stirred at 25° C. for 30 minutes to give a yellow solution. The reaction mixture was concentrated under reduced pressure to give 4-(3-fluoroazetidin-3-yl)benzonitrile (0.16 g, crude) as a yellow oil. LCMS: (ESI) m/z [M+H]⁺=177.3

Synthesis of 2-(4-amino-[1,1′-biphenyl]-3-yl)propanamide

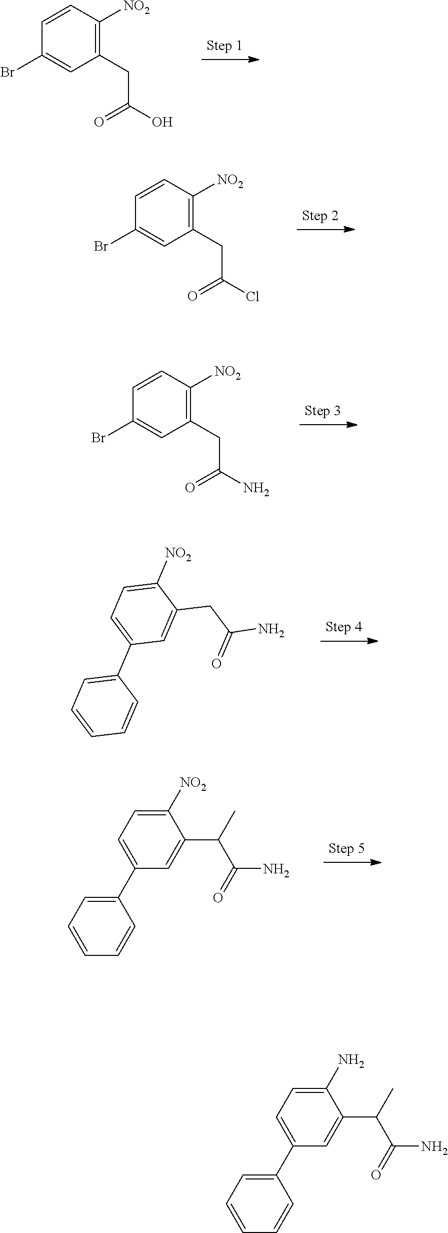 [SMILES: O=C(O)Cc1cc(Br)ccc1[N+](=O)[O-] ; O=C(Cl)Cc1cc(Br)ccc1[N+](=O)[O-] ; NC(=O)Cc1cc(Br)ccc1[N+](=O)[O-] ; NC(=O)Cc1cc(-c2ccccc2)ccc1[N+](=O)[O-] ; CC(C(N)=O)c1cc(-c2ccccc2)ccc1[N+](=O)[O-] ; CC(C(N)=O)c1cc(-c2ccccc2)ccc1N]

Step 1: Preparation of 2-(5-bromo-2-nitrophenyl)acetyl chloride

A solution of 2-(5-bromo-2-nitrophenyl)acetic acid (1.0 g, 3.8 mmol, 1 eq) in SOCl₂ was stirred at 80° C. for 1 h. The reaction mixture was concentrated under reduced pressure to give 2-(5-bromo-2-nitrophenyl)acetyl chloride (1 g, crude) as a yellow oil. LCMS: (ESI) m/z (M+MeOH)⁺=273.9.

Step 2: Preparation of 2-(5-bromo-2-nitrophenyl)acetamide

To a solution of 2-(5-bromo-2-nitrophenyl)acetyl chloride (1.0 g, 3.6 mmol, 1 eq) in THF (20 mL) was added dropwise NH₃—H₂O (0.63 g, 17 mmol, 5 eq) at 0° C., and the reaction mixture was stirred at 25° C. for 2 h. The reaction mixture was concentrated under reduced pressure to give residue. The residue was purified by column chromatography to give 2-(5-bromo-2-nitrophenyl)acetamide (0.8 g, 86% yield) as a white solid. LCMS: (ESI) m/z [M+H]⁺=258.9.

Step 3: Preparation of 2-(4-nitro-[1,1′-biphenyl]-3-yl)acetamide

To a solution of 2-(5-bromo-2-nitrophenyl)acetamide (0.8 g, 3.1 mmol, 1 eq) in dioxane (10 mL) and H₂O (1 mL) was added Na₂CO₃ (0.65 g, 6.2 mmol, 2 eq) and phenylboronic acid (0.45 g, 3.7 mmol, 1.2 eq), then Pd(dppf)Cl₂ (0.22 g, 0.31 mmol, 0.1 eq) was added under N₂, the mixture was stirred at 100° C. for 12 h. The reaction mixture was concentrated under reduced pressure to give a residue. The residue was purified by column chromatography to give 2-(4-nitro-[1,1′-biphenyl]-3-yl)acetamide (0.5 g, 63% yield) as a white solid. LCMS: (ESI) m/z [M+H]⁺=257.0.

Step 4: Preparation of 2-(4-nitro-[1,1′-biphenyl]-3-yl)propenamide

To a solution of 2-(4-nitro-[1,1′-biphenyl]-3-yl)acetamide (50 mg, 0.19 mmol, 1 eq) in dimethylformamide (10 mL) was added cesium carbonate (0.13 g, 0.38 mmol, 2 eq) and methyl iodide (41 mg, 0.29 mmol, 1.5 eq), the mixture was stirred at 80° C. under microwave irradiation for 2 h. The reaction mixture was concentrated under reduced pressure to give a residue. The residue was purified by column chromatography to give 2-(4-nitro-[1,1′-biphenyl]-3-yl)propanamide (34 mg, 64% yield) as a white solid. LCMS: (ESI) m/z [M+H]⁺=271.2.

Step 5: Preparation of 2-(4-amino-[1,1′-biphenyl]-3-yl)propanamide

To a solution of 2-(4-nitro-[1,1′-biphenyl]-3-yl)propanamide (30 mg, 0.11 mmol, 1 eq) in ethyl alcohol (0.4 mL) and water (0.1 mL) was added ammonium chloride (18 mg, 0.33 mmol, 3 eq) and iron (61 mg, 1.1 mmol, 10 eq). The mixture was stirred at 25° C. for 4 h. The reaction mixture was filtered and the filtrate was concentrated to give 2-(4-amino-[1,1′-biphenyl]-3-yl)propanamide (30 mg, crude) as a white solid. LCMS: (ESI) m/z [M+H]⁺=241.1

Synthesis of (R)-azetidin-3-yl(3-methoxypyrrolidin-1-yl)methanone

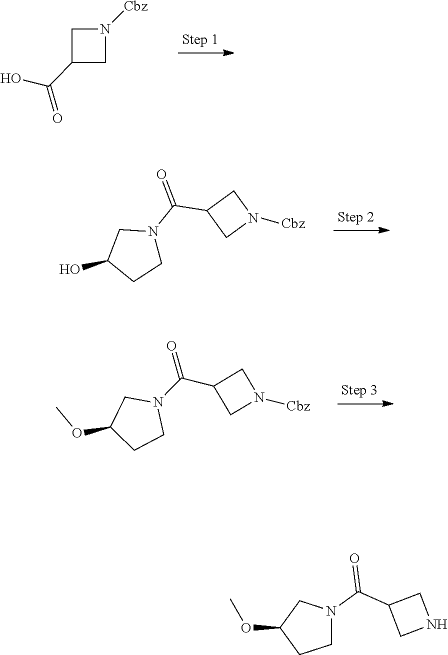 [SMILES: O=C(O)C1CN(C(=O)OCc2ccccc2)C1 ; O=C(OCc1ccccc1)N1CC(C(=O)N2CC[C@@H](O)C2)C1 ; CO[C@@H]1CCN(C(=O)C2CN(C(=O)OCc3ccccc3)C2)C1 ; CO[C@@H]1CCN(C(=O)C2CNC2)C1]

Step 1: Preparation of benzyl (R)-3-(3-hydroxypyrrolidine-1-carbonyl)azetidine-1-carboxylate

To a solution of 1-((benzyloxy)carbonyl)azetidine-3-carboxylic acid (0.5 g, 2.1 mmol, 1 eq) in DMF (5 mL) was added HATU (1.2 g, 3.2 mmol, 1.5 eq), DIEA (0.82 g, 6.3 mmol, 3 eq) and (R)-pyrrolidin-3-ol (0.18 g, 2.1 mmol, 1 eq), the mixture was stirred at 25° C. for 2 h to give a yellow solution. The mixture was diluted with water (20 mL) and extracted with EtOAc (20 mL×2), the combined organic layers were washed with saturated brine (20 mL×2), dried over Na₂SO₄, filtered, and concentrated under reduced pressure to give a residue. The residue was purified by flash silica gel chromatography to give benzyl (R)-3-(3-hydroxypyrrolidine-1-carbonyl)azetidine-1-carboxylate (0.55 g, 85% yield) as a yellow oil. ¹H NMR (400 MHz, CDCl₃) δ 7.44-7.30 (m, 5H), 5.16-5.02 (m, 2H), 4.59-4.49 (m, 1H), 4.37-4.06 (m, 5H), 3.74-3.16 (m, 6H).

Step 2: Preparation of benzyl (R)-3-(3-methoxypyrrolidine-1-carbonyl)azetidine-1-carboxylate

To a solution of benzyl (R)-3-(3-hydroxypyrrolidine-1-carbonyl)azetidine-1-carboxylate (50 mg, 0.16 mmol, 1 eq) in THF (1 mL) was added NaH (13 mg, 0.33 mmol, 2 eq) at 0° C. and stirred for 10 minutes under N₂, then added methyl iodide (47 mg, 0.33 mmol, 2 eq). The mixture was stirred at 25° C. for 2 h to give a white suspension. The reaction mixture was quenched by H₂O (1 mL), then was concentrated under reduced pressure to give a residue. The residue was purified by flash silica gel chromatography to give benzyl (R)-3-(3-methoxypyrrolidine-1-carbonyl)azetidine-1-carboxylate (22 mg, 42% yield) as a yellow oil. ¹H NMR (400 MHz, CDCl₃) δ 7.44-7.30 (m, 5H), 5.11 (s, 2H), 4.17-8.15 (m, 2H), 4.07-3.92 (m, 1H), 3.79-3.38 (m, 6H), 3.34 (d, J=3.2 Hz, 3H), 2.22-1.79 (m, 2H), 1.77-1.41 (m, 1H).

Step 3: Preparation of (R)-azetidin-3-yl(3-methoxypyrrolidin-1-yl)methanone

To a solution of benzyl (R)-3-(3-methoxypyrrolidine-1-carbonyl)azetidine-1-carboxylate (50 mg, 0.16 mmol, 1 eq) in MeOH (2.5 mL) was added Pd/C (10 mg) under N₂. The mixture was stirred at 25° C. for 16 h under H₂ (15 psi) to give a black suspension. The reaction mixture was filtered and the filtrate was concentrated to give (R)-azetidin-3-yl(3-methoxypyrrolidin-1-yl)methanone (40 mg, crude) as a yellow oil.

Synthesis of (R)-1-(1H-pyrazol-4-yl)pyrrolidin-3-amine

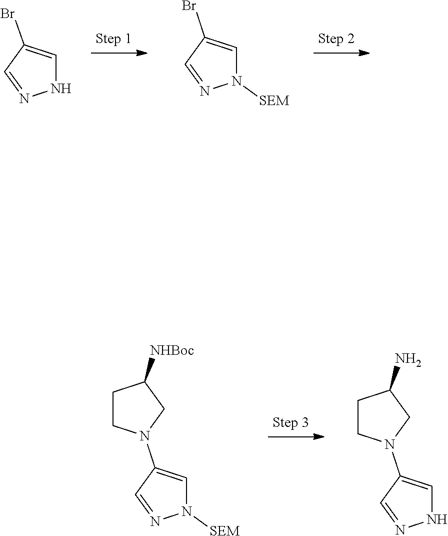 [SMILES: Brc1cn[nH]c1 ; C[Si](C)(C)CCOCn1cc(Br)cn1 ; CC(C)(C)OC(=O)N[C@@H]1CCN(c2cnn(COCC[Si](C)(C)C)c2)C1 ; N[C@@H]1CCN(c2cn[nH]c2)C1]

Step 1: Preparation of 4-bromo-1-((2-(trimethylsilyl)ethoxy)methyl)-1H-pyrazole

To a solution of 4-bromo-1H-pyrazole (2.0 g, 14 mmol, 1 eq) in THF (30 mL) was cooled to 0° C., then sodium hydride (1.1 g, 27 mmol, 1.5 eq) was added at 0° C. under N₂. The mixture was stirred at 0° C. for 30 min then SEMCl (3.4 g, 20 mmol, 1.5 eq) was added dropwise at 0° C. The mixture was stirred at 25° C. for 12 h under N₂ to give a yellow solution. The reaction mixture was quenched by H₂O (20 mL) at 0° C., then was diluted with water (50 mL) and extracted with EtOAc (50 mL×2), the combined organic layers were washed with saturated brine (50 mL×2), dried over Na₂SO₄, filtered, and concentrated under reduced pressure to give a residue. The residue was purified by column chromatography (SiO₂, Petroleum ether/EtOAc=1:0 to 95:5) to give 4-bromo-1-((2-(trimethylsilyl)ethoxy)methyl)-1H-pyrazole (1.2 g, 31% yield) as a white solid. ¹H NMR (400 MHz, METHANOL-d₄) δ 7.93 (s, 1H), 7.54 (s, 1H), 5.40 (s, 2H), 3.62-3.50 (m, 2H), 0.91-0.83 (m, 2H), 0.05-0.09 (m, 9H).

Step 2: Preparation of (R)-tert-butyl (1-(1-((2-(trimethylsilyl)ethoxy)methyl)-1H-pyrazol-4-yl)pyrrolidin-3-yl)carbamate

To a solution of 4-bromo-1-((2-(trimethylsilyl)ethoxy)methyl)-1H-pyrazole (0.50 g, 1.8 mmol, 1 eq), tert-butyl (R)-pyrrolidin-3-ylcarbamate (0.67 g, 3.6 mmol, 2 eq), potassium tert-butoxide (0.40 g, 3.6 mmol, 2 eq) in ortho-xylene (10 mL) was degassed and purged with N₂ for three times, and then the mixture was added Pd₂(dba)₃ (0.16 g, 0.18 mmol, 0.1 eq) and t-BuDavephos (0.12 g, 0.36 mmol, 0.2 eq). The mixture was stirred at 90° C. for 12 h to give a black solution under. The mixture was diluted with water (50 mL) and extracted with EtOAc (50 mL×2), the combined organic layers were washed with saturated brine (50 mL×2), dried over Na₂SO₄, filtered, and concentrated under reduced pressure to give a residue. The residue was purified by column chromatography (SiO₂, Petroleum ether/EtOAc=1:0 to 6:1) to give (R)-tert-butyl (1-(1-((2-(trimethylsilyl)ethoxy)methyl)-1H-pyrazol-4-yl)pyrrolidin-3-yl)carbamate (60 mg, 8.7% yield) as a white solid. ¹H NMR (400 MHz, METHANOL-d₄) δ 7.26-7.13 (m, 2H), 5.38-5.25 (m, 2H), 4.24-4.14 (m, 1H), 3.57-3.48 (m, 2H), 3.21 (m, 1H), 3.05 (m, 1H), 2.93-2.89 (m, 1H), 2.33-2.22 (m, 1H), 1.90-1.79 (m, 1H), 1.48-1.41 (m, 9H), 1.36-1.27 (m, 1H), 0.92-0.82 (m, 2H), 0.03-0.06 (m, 9H).

Step 3: Preparation of (R)-1-(1H-pyrazol-4-yl)pyrrolidin-3-amine

A solution of (R)-tert-butyl (1-(1-((2-(trimethylsilyl)ethoxy)methyl)-1H-pyrazol-4-yl)pyrrolidin-3-yl)carbamate (30 mg, 78 μmol, 1 eq) in DCM (2 mL) and TFA (2 mL) was stirred at 25° C. for 1 h. The reaction mixture was concentrated under reduced pressure to give (R)-1-(1H-pyrazol-4-yl)pyrrolidin-3-amine (30 mg, crude) as a yellow oil. LCMS: (ESI) m/z [M+H]⁺=153.0

Synthesis of (R)-3-aminopyrrolidine-1-carboxamide

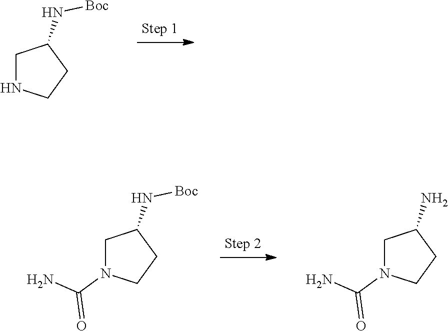 [SMILES: CC(C)(C)OC(=O)N[C@@H]1CCNC1 ; CC(C)(C)OC(=O)N[C@@H]1CCN(C(N)=O)C1 ; NC(=O)N1CC[C@@H](N)C1]

Step 1: Preparation of tert-butyl (R)-(1-carbamoylpyrrolidin-3-yl)carbamate

To a solution of tert-butyl (R)-pyrrolidin-3-ylcarbamate (1.0 g, 5.4 mmol, 1.0 eq) in 1,4-dioxane (20 mL) was added urea (1.3 g, 16 mmol, 3.0 eq), the mixture was stirred at 140° C. for 12 h to give a yellow solution. The reaction mixture was concentrated under reduced pressure to give a residue. The residue was washed by DCM and filtered. The filtrate was concentrated to give a residue. The residue was purified by flash silica gel chromatography to give tert-butyl (R)-(1-carbamoylpyrrolidin-3-yl)carbamate (0.800 g, 66% yield) as a white solid. ¹H NMR (400 MHz, CDCl₃) δ 5.06-4.06 (m, 4H), 3.62 (dd, J=6.0, 10.4 Hz, 1H), 3.48 (s, 2H), 3.25 (dd, J=3.6, 10.0 Hz, 1H), 2.28-2.08 (m, 1H), 1.90 (dd, J=6.4, 12.0 Hz, 1H), 1.45 (s, 9H)

Step 2: Preparation of (R)-3-aminopyrrolidine-1-carboxamide

A solution of tert-butyl (R)-(1-carbamoylpyrrolidin-3-yl)carbamate (0.8 g, 3.5 mmol, 1 eq) in DCM (10 mL) and trifluoroacetic acid (3 mL) was stirred at 20° C. for 1 h to give a white solution. The reaction mixture was concentrated under reduced pressure to give (R)-3-aminopyrrolidine-1-carboxamide (1 g, crude) as a yellow oil. LCMS: (ESI) m/z [M+H]⁺=130.1.

Synthesis of 1-(pyridin-2-yl)azetidin-3-amine

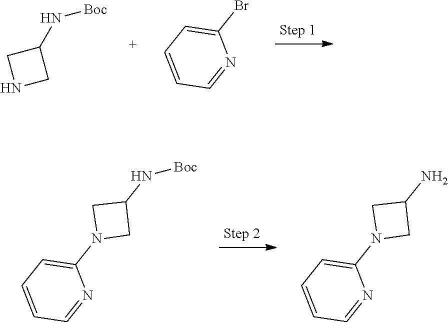 [SMILES: CC(C)(C)OC(=O)NC1CNC1 ; Brc1ccccn1 ; CC(C)(C)OC(=O)NC1CN(c2ccccn2)C1 ; NC1CN(c2ccccn2)C1]

Step 1: Preparation of tert-butyl (1-(pyridin-2-yl)azetidin-3-yl)carbamate

To a solution of 2-bromopyridine (0.50 g, 3.2 mmol, 1 eq) and tert-butyl azetidin-3-ylcarbamate (0.54 g, 3.2 mmol, 1 eq) in DMF (5 mL) was added K₂CO₃ (1.3 g, 9.5 mmol, 3 eq). The mixture was stirred at 80° C. for 16 h to give an off-white suspension. The reaction mixture was quenched by water (30 mL) and extracted with EtOAc 90 mL (30 mL×3). The combined organic layers were washed with saturated brine (30 mL), dried over Na₂SO₄, filtered, and concentrated under reduced pressure to give a residue. The residue was purified by prep-HPLC (column: YMC Triart C18 250×50 mm×7 um; mobile phase: [water (FA)-ACN]; B %: 7%-37%, 10 min) follow by lyophilization to give tert-butyl (1-(pyridin-2-yl)azetidin-3-yl)carbamate (0.12 g, 15% yield) as a white solid. LCMS: (ESI) m/z [M+H]⁺=250.1

Step 2: Preparation of 1-(pyridin-2-yl)azetidin-3-amine

A solution of tert-butyl (1-(pyridin-2-yl)azetidin-3-yl)carbamate (0.10 g, 0.40 mmol, 1 eq) in DCM (0.9 mL) and TFA (0.3 mL) was stirred at 25° C. for 1 h to give a light-yellow solution. The reaction mixture was concentrated under reduced pressure to give 1-(pyridin-2-yl)azetidin-3-amine (0.10 g, crude) as a yellow oil. LCMS: (ESI) m/z [M+H]⁺=150.2

### SFC Method:

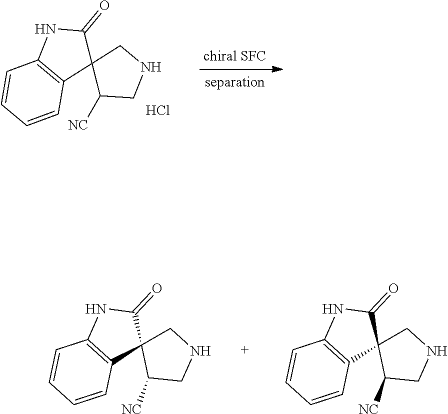 [SMILES: N#CC1CNCC12C(=O)Nc1ccccc12 ; Cl ; N#C[C@H]1CNC[C@@]12C(=O)Nc1ccccc12 ; N#C[C@@H]1CNC[C@]12C(=O)Nc1ccccc12]

After chiral SFC separation (column name: Cellulose 4, 15% Methanol (0.1% DEA), Flow Rate: 4 mL/min, Injected Volume: 14 μL, Outlet Pressure: 100 bar), (3R,4′R)-2-oxospiro[indoline-3,3′-pyrrolidine]-4′-carbonitrile (fastest eluting enantiomer) and (3S,4'S)-2-oxospiro[indoline-3,3′-pyrrolidine]-4′-carbonitrile (slowest eluting enantiomer) were isolated. Relative stereochemistry of oxospiro[indoline-3,3′-pyrrolidine]system is as drawn but absolute stereochemistry is unknown.

### Representative Procedures for General Schemes

### Representative Procedure: General Scheme 1 (Phosphonate Acids):

Synthesis of (difluoro(2-(((3S,6S,7aS,8aR,9aR)-3-(3-((R)-2-methylmorpholine-4-carbonyl)azetidine-1-carbonyl)-5-oxodecahydro-1H-cyclopropa[d]pyrrolo[1,2-a]azocin-6-yl)carbamoyl)benzo[b]thiophen-5-yl)methyl)phosphonic acid (Example 1)

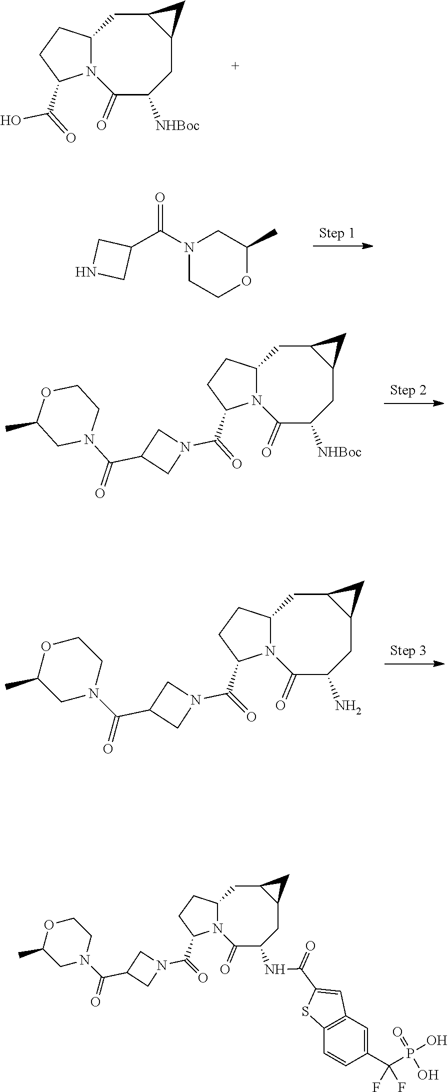 [SMILES: CC(C)(C)OC(=O)N[C@H]1C[C@@H]2C[C@@H]2C[C@H]2CC[C@@H](C(=O)O)N2C1=O ; C[C@@H]1CN(C(=O)C2CNC2)CCO1 ; C[C@@H]1CN(C(=O)C2CN(C(=O)[C@@H]3CC[C@@H]4C[C@H]5C[C@H]5C[C@H](NC(=O)OC(C)(C)C)C(=O)N43)C2)CCO1 ; C[C@@H]1CN(C(=O)C2CN(C(=O)[C@@H]3CC[C@@H]4C[C@H]5C[C@H]5C[C@H](N)C(=O)N43)C2)CCO1 ; C[C@@H]1CN(C(=O)C2CN(C(=O)[C@@H]3CC[C@@H]4C[C@H]5C[C@H]5C[C@H](NC(=O)c5cc6cc(C(F)(F)P(=O)(O)O)ccc6s5)C(=O)N43)C2)CCO1]

Step 1: Preparation of tert-butyl ((3S,6S,7aS,8aR,9aR)-3-(3-((R)-2-methylmorpholine-4-carbonyl)azetidine-1-carbonyl)-5-oxodecahydro-1H-cyclopropa[d]pyrrolo[1,2-a]azocin-6-yl)carbamate

To a solution of (3S,6S,7aS,8aR,9aR)-6-((tert-butoxycarbonyl)amino)-5-oxodecahydro-1H-cyclopropa[d]pyrrolo[1,2-a]azocine-3-carboxylic acid (50 mg, 147 μmol, 1.0 eq.), HATU (66.8 mg, 176 μmol, 1.2 eq.) and triethylamine (44.6 mg, 441 μmol, 3.0 eq.) in DMF (2 mL) was added (R)-azetidin-3-yl(2-methylmorpholino)methanone (29.6 mg, 161 μmol, 1.1 eq.) and the resulting mixture was stirred at 20° C. overnight under N₂. After completion, the reaction mixture was filtered and concentrated under reduced pressure. The residue was purified by Biotage® C18 column chromatography to get tert-butyl ((3S,6S,7aS,8aR,9aR)-3-(3-((R)-2-methylmorpholine-4-carbonyl)azetidine-1-carbonyl)-5-oxodecahydro-1H-cyclopropa[d]pyrrolo[1,2-a]azocin-6-yl)carbamate (70.0 mg, 138 μmol, 94%) as a white solid. LCMS (ESI): m/z=505.4 [M+H]⁺.

Step 2: Preparation of (5S,8S,10aR)-3-acetyl-5-amino-8-(6-phenyl-4-azaspiro[2.4]heptane-4-carbonyl)octahydropyrrolo[1,2-a][1,5]diazocin-6(1H)-one

A solution of tert-butyl ((3S,6S,7aS,8aR,9aR)-3-(3-((R)-2-methylmorpholine-4-carbonyl)azetidine-1-carbonyl)-5-oxodecahydro-1H-cyclopropa[d]pyrrolo[1,2-a]azocin-6-yl)carbamate (80 mg, 158 μmol, 1.0 eq.) in DCM (6 mL) and TFA (2 mL) was stirred at 25° C. overnight under N₂. After completion, the reaction mixture was concentrated to get crude (5S,8S,10aR)-3-acetyl-5-amino-8-(6-phenyl-4-azaspiro[2.4]heptane-4-carbonyl)octahydropyrrolo[1,2-a][1,5]diazocin-6(1H)-one (63.0 mg, quant.) as a white solid, which was used in next step directly without further purification. LCMS (ESI): m/z=405.4 [M+H]⁺.

Step 3: Preparation of (difluoro(2-(((3S,6S,7aS,8aR,9aR)-3-(3-((R)-2-methylmorpholine-4-carbonyl)azetidine-1-carbonyl)-5-oxodecahydro-1H-cyclopropa[d]pyrrolo[1,2-a]azocin-6-yl)carbamoyl)benzo[b]thiophen-5-yl)methyl)phosphonic acid

A solution of (5S,8S,10aR)-3-acetyl-5-amino-8-(6-phenyl-4-azaspiro[2.4]heptane-4-carbonyl)octahydropyrrolo[1,2-a][1,5]diazocin-6(1H)-one (7, 60 mg, 148 μmol, 1.0 eq.), triethylamine (44.8 mg, 443 μmol, 3.0 eq.), DMAP (9.03 mg, 74.0 μmol, 0.5 eq) and (difluoro(2-((perfluorophenoxy)carbonyl)benzo[b]thiophen-5-yl)methyl)phosphonic acid (83.9 mg, 177 μmol, 1.2 eq.) in DMF (2 mL) was stirred at 30° C. overnight under N₂. After completion, the reaction mixture was filtered and concentrated under reduced pressure. The residue was purified by Biotage® C18 column chromatography to get (difluoro(2-(((3S,6S,7aS,8aR,9aR)-3-(3-((R)-2-methylmorpholine-4-carbonyl)azetidine-1-carbonyl)-5-oxodecahydro-1H-cyclopropa[d]pyrrolo[1,2-a]azocin-6-yl)carbamoyl)benzo[b]thiophen-5-yl)methyl)phosphonic acid (80.0 mg, 115 μmol, 78%) as a white solid. LCMS (ESI): m/z=695.3 [M+H]⁺; ¹H NMR (400 MHz, DMSO-d₆) δ 9.00-8.84 (m, 1H), 8.34 (s, 1H), 8.13 (d, J=8.5 Hz, 1H), 8.08 (s, 1H), 7.58 (d, J=8.6 Hz, 1H), 5.00-4.86 (m, 1H), 4.68-4.46 (m, 1H), 4.40-4.25 (m, 2H), 4.22-4.09 (m, 2H), 4.05-3.85 (m, 2H), 3.81-3.63 (m, 2H), 3.48-3.27 (m, 3H), 3.11-2.98 (m, 1H), 2.78-2.62 (m, 1H), 2.39-2.28 (m, 1H), 2.18-1.96 (m, 4H), 1.91-1.75 (m, 2H), 1.70-1.55 (m, 1H), 1.42-1.27 (m, 1H), 1.12-1.01 (m, 3H), 0.84-0.66 (m, 2H), 0.03-0.04 (m, 1H).

### Representative Procedure: General Scheme 2: (Phosphonate Esters or Amides)

Synthesis of isopropyl ((difluoro(2-(((3S,6S,7aS,8aR,9aR)-3-(3-(morpholine-4-carbonyl)azetidine-1-carbonyl)-5-oxodecahydro-1H-cyclopropa[d]pyrrolo[1,2-a]azocin-6-yl)carbamoyl)benzo[b]thiophen-5-yl)methyl)(phenoxy)phosphoryl)glycinate (Example 2)

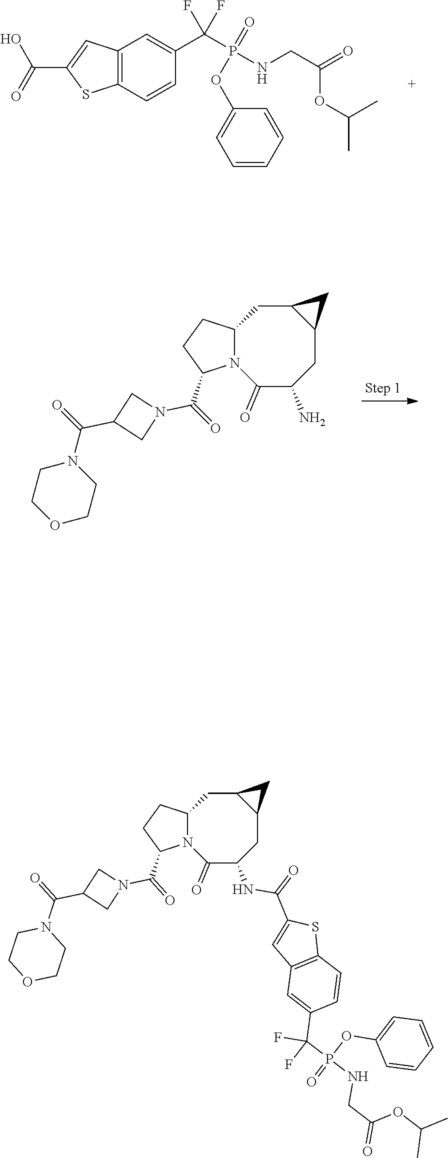 [SMILES: CC(C)OC(=O)CNP(=O)(Oc1ccccc1)C(F)(F)c1ccc2sc(C(=O)O)cc2c1 ; N[C@H]1C[C@@H]2C[C@@H]2C[C@H]2CC[C@@H](C(=O)N3CC(C(=O)N4CCOCC4)C3)N2C1=O ; CC(C)OC(=O)CNP(=O)(Oc1ccccc1)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1]

To a solution of (3S,6S,7aS,8aR,9aR)-6-amino-3-(3-(morpholine-4-carbonyl)azetidine-1-carbonyl)decahydro-5H-cyclopropa[d]pyrrolo[1,2-a]azocin-5-one (50 mg, 99.1 μmol, 1 eq), DIPEA (99.2 mg, 768 μmol, 7.75 eq) and 5-(difluoro(((2-isopropoxy-2-oxoethyl)amino)(phenoxy)phosphoryl)methyl)benzo[b]thiophene-2-carboxylic acid (73.9 mg, 153 μmol, 1.54 eq) in DMF (3 mL) was added HATU (63.1 mg, 166 μmol, 1.67 eq). The reaction was stirred at room temperature for 1 h. The reaction mixture was directly purified by reverse phase chromatography on a 50 g C18 cartridge eluting with 5-100% MeCN in water to give isopropyl ((difluoro(2-(((3S,6S,7aS,8aR,9aR)-3-(3-(morpholine-4-carbonyl)azetidine-1-carbonyl)-5-oxodecahydro-1H-cyclopropa[d]pyrrolo[1,2-a]azocin-6-yl)carbamoyl)benzo[b]thiophen-5-yl)methyl)(phenoxy)phosphoryl)glycinate (88.2 mg, 103 μmol, 104%) as a white solid. LCMS: (ESI) m/z=856.1 [M+H]⁺; ¹H NMR (400 MHz, CD₃OD) δ 8.23-8.12 (m, 2H), 8.07-7.98 (m, 1H), 7.75-7.69 (m, 1H), 7.36-7.29 (m, 2H), 7.23-7.15 (m, 3H), 5.07-4.92 (m, 2H), 4.73-4.70 (m, 1H), 4.54-4.37 (m, 2H), 4.33-4.16 (m, 2H), 4.11-4.02 (m, 1H), 3.82-3.71 (m, 1H), 3.71-3.44 (m, 8H), 3.38-3.34 (m, 1H), 3.30-3.20 (m, 1H), 2.50-2.38 (m, 1H), 2.35-2.06 (m, 4H), 2.04-1.92 (m, 2H), 1.78-1.67 (m, 1H), 1.45-1.36 (m, 1H), 1.20-1.16 (m, 6H), 0.98-0.89 (m, 1H), 0.88-0.79 (m, 1H), 0.10-0.02 (m, 1H).

### Representative Procedure: General Scheme 3 (Phosphonate Acids):

Synthesis of ((2-(((3S,6S,7aS,8aR,9aR)-3-([1,1′-biphenyl]-4-ylcarbamoyl)-5-oxodecahydro-1H-cyclopropa[d]pyrrolo[1,2-a]azocin-6-yl)carbamoyl)benzo[b]thiophen-5-yl)difluoromethyl)phosphonic acid (Example 3)

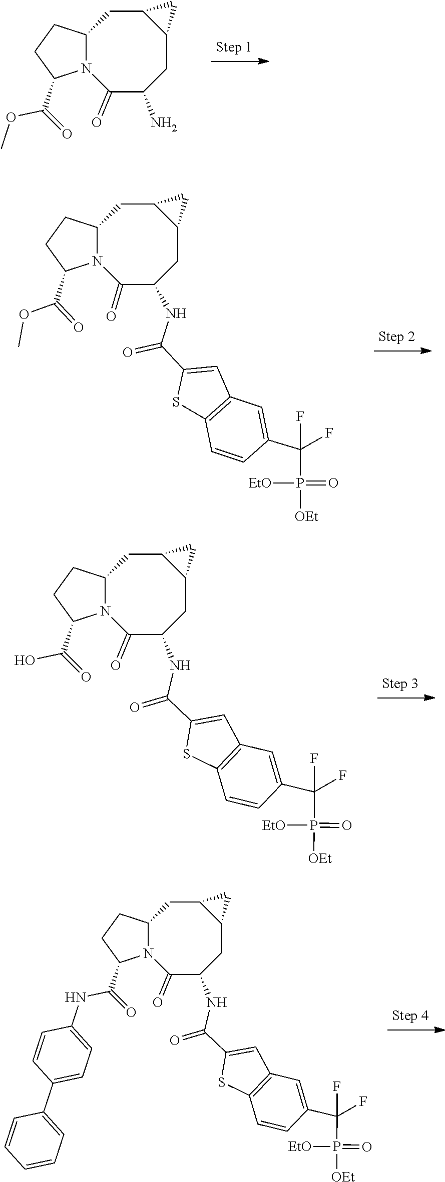 [SMILES: COC(=O)[C@@H]1CC[C@@H]2C[C@@H]3C[C@@H]3C[C@H](N)C(=O)N21 ; CCOP(=O)(OCC)C(F)(F)c1ccc2sc(C(=O)N[C@H]3C[C@H]4C[C@H]4C[C@H]4CC[C@@H](C(=O)OC)N4C3=O)cc2c1 ; CCOP(=O)(OCC)C(F)(F)c1ccc2sc(C(=O)N[C@H]3C[C@H]4C[C@H]4C[C@H]4CC[C@@H](C(=O)O)N4C3=O)cc2c1 ; CCOP(=O)(OCC)C(F)(F)c1ccc2sc(C(=O)N[C@H]3C[C@H]4C[C@H]4C[C@H]4CC[C@@H](C(=O)Nc5ccc(-c6ccccc6)cc5)N4C3=O)cc2c1]

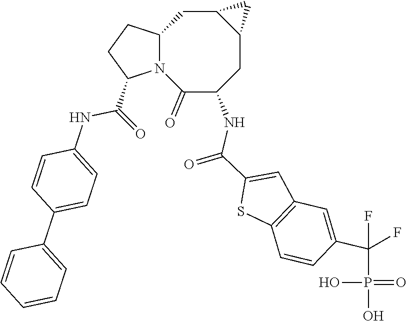 [SMILES: O=C(N[C@H]1C[C@H]2C[C@H]2C[C@H]2CC[C@@H](C(=O)Nc3ccc(-c4ccccc4)cc3)N2C1=O)c1cc2cc(C(F)(F)P(=O)(O)O)ccc2s1]

Step 1: Preparation of methyl (3S,6S,7aR,8aS,9aR)-6-(5-((diethoxyphosphoryl)difluoromethyl)benzo[b]thiophene-2-carboxamido)-5-oxodecahydro-1H-cyclopropa[d]pyrrolo[1,2-a]azocine-3-carboxylate

To a solution of 5-((diethoxyphosphoryl)difluoromethyl)benzo[b]thiophene-2-carboxylic acid (29 mg, 0.79 mmol, 1 eq) in DMF (1 mL) was added HATU (45 mg, 1.2 mmol, 1.5 eq), then a solution of methyl (3S,6S,7aR,8aS,9aR)-6-amino-5-oxodecahydro-1H-cyclopropa[d]pyrrolo[1,2-a]azocine-3-carboxylate (20 mg, 0.79 mmol, 1.2 eq) in DMF (1 mL) and DIEA (41 mg, 3.2 mmol, 4 eq) was added, the mixture was stirred at 25° C. for 1 h. The reaction mixture was quench with water (10 mL) and extracted with EtOAc (10 mL×3), the combined organic layers were washed with saturated brine (10 mL×3), dried over Na₂SO₄, filtered, and concentrated under reduced pressure to give a residue. The residue was purified by column chromatography (SiO₂, Petroleum ether/EtOAc=1:1 to 15:85) to give methyl (3S,6S,7aR,8aS,9aR)-6-(5-((diethoxyphosphoryl)difluoromethyl)benzo[b]thiophene-2-carboxamido)-5-oxodecahydro-1H-cyclopropa[d]pyrrolo[1,2-a]azocine-3-carboxylate (15 mg, 41% yield) as a yellow solid. LCMS: m/z [M+H]⁺=599.4.

Step 2: Preparation of (3S,6S,7aR,8aS,9aR)-6-(5-((diethoxyphosphoryl)difluoromethyl)benzo[b]thiophene-2-carboxamido)-5-oxodecahydro-1H-cyclopropa[d]pyrrolo[1,2-a]azocine-3-carboxylic acid

To a solution of methyl (3S,6S,7aR,8aS,9aR)-6-(5-((diethoxyphosphoryl)difluoromethyl)benzo[b]thiophene-2-carboxamido)-5-oxodecahydro-1H-cyclopropa[d]pyrrolo[1,2-a]azocine-3-carboxylate (15 mg, 0.25 mmol, 1 eq) in ACN (0.9 mL) and 2 N HCl (0.3 mL), the mixture was stirred at 70° C. for 5 h. The reaction mixture was filtered and the filter residue was purified by prep-HPLC (column: Phenomenex Luna C18 150×25 mm×10 um, mobile phase: water (0.1% TFA)-ACN; B %: 28%-58%, 10 min) follow by lyophilization to give (3S,6S,7aR,8aS,9aR)-6-(5-((diethoxyphosphoryl)difluoromethyl)benzo[b]thiophene-2-carboxamido)-5-oxodecahydro-1H-cyclopropa[d]pyrrolo[1,2-a]azocine-3-carboxylic acid (4 mg, 43% yield) as a yellow oil. ¹H NMR (400 MHz, CDCl₃) δ 8.11 (s, 1H), 7.95 (m, 2H), 7.85 (d, J=5.6 Hz, 1H), 7.66 (d, J=8.4 Hz, 1H), 4.96 (d, J=5.2 Hz, 1H), 4.56-4.45 (m, 1H), 4.32-4.10 (m, 5H), 2.39-2.27 (m, 2H), 2.24-2.12 (m, 2H), 2.10-2.00 (m, 1H), 1.99-1.88 (m, 1H), 1.82-1.72 (m, 1H), 1.72-1.64 (m, 2H), 1.37-1.29 (m, 6H), 1.27-1.18 (m, 1H), 0.87-0.75 (m, 1H), 0.10 (d, J=4.8 Hz, 1H).

Step 3: Preparation of diethyl ((2-(((3S,6S,7aR,8aS,9aR)-3-([1,1′-biphenyl]-4-ylcarbamoyl)-5-oxodecahydro-1H-cyclopropa[d]pyrrolo[1,2-a]azocin-6-yl)carbamoyl)benzo[b]thiophen-5-yl)difluoromethyl)phosphonate

To a solution of (3S,6S,7aR,8aS,9aR)-6-(5-((diethoxyphosphoryl)difluoromethyl)benzo[b]thiophene-2-carboxamido)-5-oxodecahydro-1H-cyclopropa[d]pyrrolo[1,2-a]azocine-3-carboxylic acid (4 mg, 6.8 mol, 1 eq) in DMF (0.3 mL) was added HATU (3.8 mg, 10 mol, 1.5 eq), then was added the solution of [1,1′-biphenyl]-4-amine (1.4 mg, 8.2 mol, 1.2 eq) in DMF (0.3 mL) and DIEA (4.0 mg, 0.33 mmol, 4 eq), the mixture was stirred at 25° C. for 1 h. The mixture was purified directly by column chromatography (SiO₂, Petroleum ether/EtOAc=10:1 to 3:1) to give diethyl ((2-(((3S,6S,7aR,8aS,9aR)-3-([1,1′-biphenyl]-4-ylcarbamoyl)-5-oxodecahydro-1H-cyclopropa[d]pyrrolo[1,2-a]azocin-6-yl)carbamoyl)benzo[b]thiophen-5-yl)difluoromethyl)phosphonate (12 mg, crude) as a yellow oil. LCMS: m/z [M+H]⁺=736.2

Step 4: Preparation of ((2-(((3S,6S,7aR,8aS,9aR)-3-([1,1′-biphenyl]-4-ylcarbamoyl)-5-oxodecahydro-1H-cyclopropa[d]pyrrolo[1,2-a]azocin-6-yl)carbamoyl)benzo[b]thiophen-5-yl)difluoromethyl)phosphonic acid

To a solution of diethyl ((2-(((3S,6S,7aR,8aS,9aR)-3-([1,1′-biphenyl]-4-ylcarbamoyl)-5-oxodecahydro-1H-cyclopropa[d]pyrrolo[1,2-a]azocin-6-yl)carbamoyl)benzo[b]thiophen-5-yl)difluoromethyl)phosphonate (12 mg, 0.16 mmol, 1 eq) in DCM (0.5 mL) was added BSTFA (25 mg, 0.96 mmol, 6 eq) and dropwise TMSI (13 mg, 0.64 mmol, 4 eq) in DCM (0.5 ml) at 0° C., the mixture was stirred at 0° C. for 15 min. The reaction mixture was concentrated under reduced pressure to give a residue. The residue was purified by prep-HPLC (column: Phenomenex Luna C18 150×25 mm×10 um, mobile phase: water (0.1% TFA)-ACN; B %: 40%-70%, 10 min) follow by lyophilization to give ((2-(((3S,6S,7aR,8aS,9aR)-3-([1,1′-biphenyl]-4-ylcarbamoyl)-5-oxodecahydro-1H-cyclopropa[d]pyrrolo[1,2-a]azocin-6-yl)carbamoyl)benzo[b]thiophen-5-yl)difluoromethyl)phosphonic acid (1.22 mg, 9.45% yield) as a white solid. LCMS: (ESI) m/z [M+H]⁺=680; ¹H NMR (400 MHz, CD₃OD) δ 8.18-8.08 (m, 2H), 7.98 (d, J=8.4 Hz, 1H), 7.69 (d, J=8.4 Hz, 1H), 7.66-7.61 (m, 2H), 7.61-7.54 (m, 4H), 7.41 (t, J=7.6 Hz, 2H), 7.33-7.26 (m, 1H), 5.00 (d, J=4.4 Hz, 1H), 4.55 (t, J=8.4 Hz, 1H), 4.37 (t, J=7.6 Hz, 1H), 2.42-2.31 (m, 2H), 2.27-2.14 (m, 2H), 2.12-2.04 (m, 1H), 2.03-1.96 (m, 2H), 1.89-1.76 (m, 1H), 1.54-1.41 (m, 1H), 1.38-1.23 (m, 3H), 0.92-0.84 (m, 1H), 0.29-0.22 (m, 1H)

### Specific Analog Syntheses

Synthesis of (((difluoro(2-(((3S,6S,7aS,8aR,9aR)-3-(3-(morpholine-4-carbonyl)azetidine-1-carbonyl)-5-oxodecahydro-1H-cyclopropa[d]pyrrolo[1,2-a]azocin-6-yl)carbamoyl)benzo[b]thiophen-5-yl)methyl)phosphoryl)bis(oxy))bis(methylene) dipentanoate (Example 4)

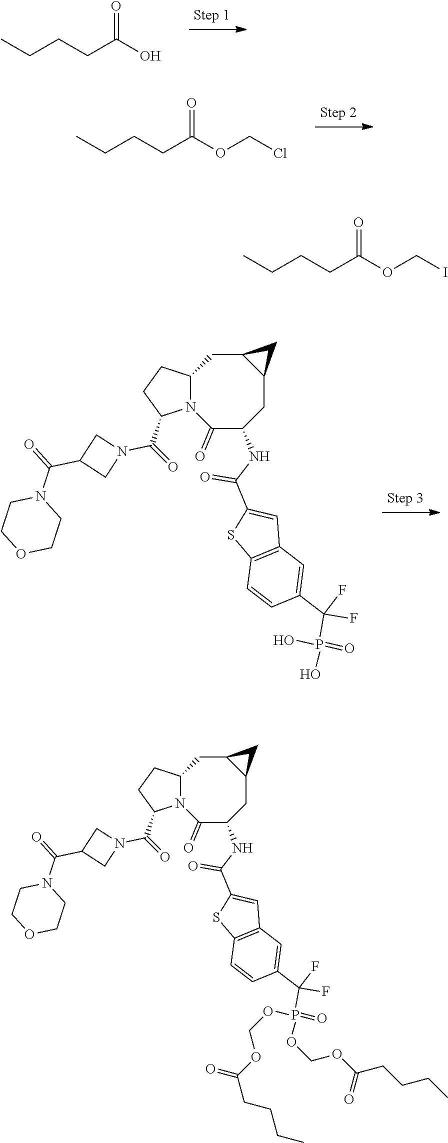

### Step 1: Preparation of Chloromethyl Pentanoate

To a solution of pentanoic acid (1, 1 g, 9.79 mmol, 1.0 eq.), tetrabutylammonium hydrogen sulfate (332 mg, 979 μmol, 0.1 eq.) and chloromethyl sulfurochloridate (1.76 g, 10.7 mmol, 1.1 eq.) in DCM (10 mL) was added a solution of NaHCO₃ (1.63 g, 19.5 mmol, 2.0 eq.) in water (10 mL) dropwise at 0° C., then the reaction was allowed to warm to room temperature and stirred overnight under N₂. After completion, the reaction mixture was extracted with dichloromethane (20 mL×3). The organic layers were combined, dried over anhydrous Na₂SO₄, and concentrated under reduced pressure. The residue was purified by flash chromatography on silica gel to get chloromethyl pentanoate (1.40 g, 9.29 mmol, 95% yield) as a clear oil. ¹H NMR (400 MHz, CDCl₃) δ 5.71 (s, 2H), 2.39 (t, J=7.5 Hz, 2H), 1.69-1.60 (m, 2H), 1.41-1.33 (m, 2H), 0.93 (t, J=7.4 Hz, 3H).

### Step 2: Preparation of Iodomethyl Pentanoate

A solution of chloromethyl pentanoate (400 mg, 2.65 mmol, 1.0 eq.) and NaI (794 mg, 5.30 mmol, 2.0 eq.) in MeCN (10 mL) was stirred at 40° C. overnight under N₂. After completion, the reaction mixture was filtered, and the filtrate was concentrated under reduced pressure. The residue was purified by flash chromatography on silica gel to get iodomethyl pentanoate (3, 100 mg, 413 μmol, 16%) as a yellow oil. ¹H NMR (400 MHz, CDCl₃) δ 5.91 (s, 2H), 2.34 (t, J=7.5 Hz, 2H), 1.68-1.58 (m, 2H), 1.41-1.31 (m, 2H), 0.92 (t, J=7.3 Hz, 3H).

Step 3: Preparation of (((difluoro(2-(((3S,6S,7aS,8aR,9aR)-3-(3-(morpholine-4-carbonyl)azetidine-1-carbonyl)-5-oxodecahydro-1H-cyclopropa[d]pyrrolo[1,2-a]azocin-6-yl)carbamoyl)benzo[b]thiophen-5-yl)methyl)phosphoryl)bis(oxy))bis(methylene) dipentanoate

A solution of NaOH (7.03 mg, 176 μmol, 2.0 eq.) in water (2 mL) was added dropwise to a stirred suspension of (difluoro(2-(((3S,6S,7aS,8aR,9aR)-3-(3-(morpholine-4-carbonyl)azetidine-1-carbonyl)-5-oxodecahydro-1H-cyclopropa[d]pyrrolo[1,2-a]azocin-6-yl)carbamoyl)benzo[b]thiophen-5-yl)methyl)phosphonic acid (60 mg, 88.1 μmol, 1.0 eq.) in H₂O (6 mL). When the mixture became clear (pH˜9), AgNO₃ (32.7 mg, 193 μmol, 2.2 eq.) was added. After stirring at 0° C. for 2 h, the gray precipitate was collected by filtration and dried under vacuum. The powder was suspended in dry toluene (1 mL) and iodomethyl pentanoate (3, 63.9 g, 264 μmol, 3.0 eq.) was added. The mixture was stirred at room temperature for 24 h. After filtration, the solvent was removed in vacuo. The crude residue was directly purified by flash chromatography on silica gel to give (((difluoro(2-(((3S,6S,7aS,8aR,9aR)-3-(3-(morpholine-4-carbonyl)azetidine-1-carbonyl)-5-oxodecahydro-1H-cyclopropa[d]pyrrolo[1,2-a]azocin-6-yl)carbamoyl)benzo[b]thiophen-5-yl)methyl)phosphoryl)bis(oxy))bis(methylene) dipentanoate (ST-214-2, 2.00 mg, 2.20 μmol, 3% yield) as a white solid. LCMS (ESI): m/z=909.3 [M+H]⁺; ¹H NMR (400 MHz, DMSO-d₆) δ 8.99 (dd, J=22.8, 7.5 Hz, 1H), 8.39-8.00 (m, 3H), 7.55 (d, J=8.6 Hz, 1H), 5.86-5.36 (m, 4H), 4.99-4.89 (m, 1H), 4.68-4.50 (m, 1H), 4.37-4.24 (m, 2H), 4.20-4.11 (m, 1H), 4.05-3.85 (m, 2H), 3.75-3.64 (m, 1H), 3.56-3.50 (m, 4H), 3.48-3.44 (m, 2H), 3.28-3.23 (m, 2H), 2.34-2.24 (m, 4H), 2.17-1.94 (m, 4H), 1.90-1.75 (m, 2H), 1.68-1.57 (m, 1H), 1.54-1.40 (m, 4H), 1.36-1.19 (m, 6H), 0.89-0.67 (m, 8H), 0.01-−0.02 (m, 1H).

Preparation of isopropyl ((R)-((2-(((3S,6S,7aS,8aR,9aR)-3-(3-(morpholine-4-carbonyl)azetidine-1-carbonyl)-5-oxodecahydro-1H-cyclopropa[d]pyrrolo[1,2-a]azocin-6-yl)carbamoyl)benzo[b]thiophen-5-yl)methyl)(phenoxy)phosphoryl)-L-alaninate and isopropyl ((S)-((2-(((3S,6S,7aS,8aR,9aR)-3-(3-(morpholine-4-carbonyl)azetidine-1-carbonyl)-5-oxodecahydro-1H-cyclopropa[d]pyrrolo[1,2-a]azocin-6-yl)carbamoyl)benzo[b]thiophen-5-yl)methyl)(phenoxy)phosphoryl)-L-alaninate (Example 5 and Example 6)

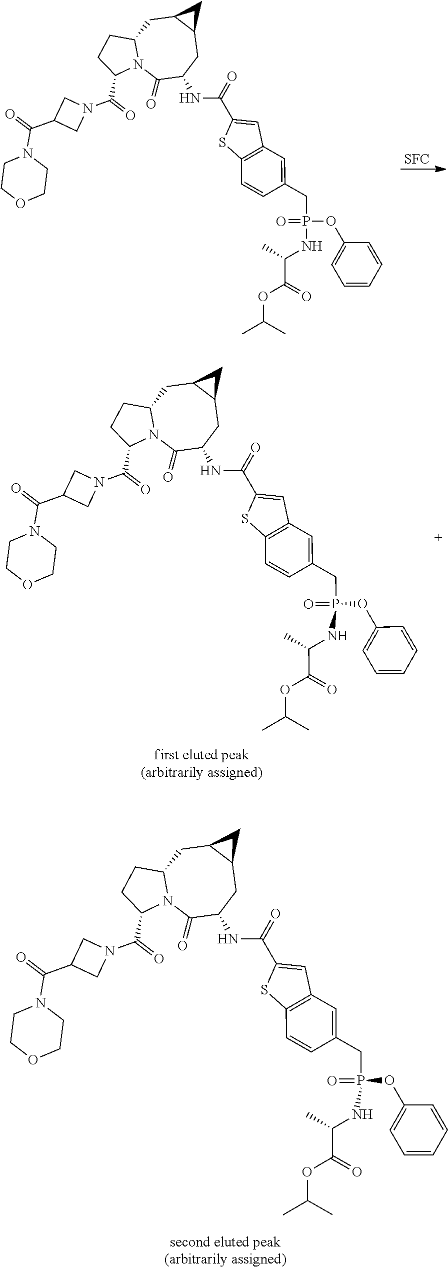 [SMILES: CC(C)OC(=O)[C@H](C)NP(=O)(Cc1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1)Oc1ccccc1 ; "SFC" ; CC(C)OC(=O)[C@H](C)N[P@@](=O)(Cc1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1)Oc1ccccc1 ; CC(C)OC(=O)[C@H](C)N[P@](=O)(Cc1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1)Oc1ccccc1]

Preparative separation method: Instrument: Waters Thar 80 preparative SFC, Column: ChiralPak IB, 250×21.2 mm I.D., 5 μm; Mobile phase: A for CO₂ and B for MeOH+0.1% NH₃H₂O; Gradient: B 35%; Flow rate: 40 mL/min; Back pressure: 100 bar; Column temperature: 35° C., Wavelength: 220 nm, Cycle-time: 4 min

Isopropyl (((2-(((3S,6S,7aS,8aR,9aR)-3-(3-(morpholine-4-carbonyl)azetidine-1-carbonyl)-5-oxodecahydro-1H-cyclopropa[d]pyrrolo[1,2-a]azocin-6-yl)carbamoyl)benzo[b]thiophen-5-yl)methyl)(phenoxy)phosphoryl)-L-alaninate (60 mg, 71.9 μmol, 1.0 eq.) was purified by prep-SFC to yield:

Peak 1: Isopropyl ((R)-((2-(((3S,6S,7aS,8aR,9aR)-3-(3-(morpholine-4-carbonyl)azetidine-1-carbonyl)-5-oxodecahydro-1H-cyclopropa[d]pyrrolo[1,2-a]azocin-6-yl)carbamoyl)benzo[b]thiophen-5-yl)methyl)(phenoxy)phosphoryl)-L-alaninate or isopropyl ((S)-((2-(((3S,6S,7aS,8aR,9aR)-3-(3-(morpholine-4-carbonyl)azetidine-1-carbonyl)-5-oxodecahydro-1H-cyclopropa[d]pyrrolo[1,2-a]azocin-6-yl)carbamoyl)benzo[b]thiophen-5-yl)methyl)(phenoxy)phosphoryl)-L-alaninate (20.0 mg, 24.0 μmol, 99.9% ee, 33% yield) as a white solid Peak 1 data (Example 5), P-chirality arbitrarily assigned LCMS (ESI): m/z=834.4 [M+H]⁺; ¹H NMR (400 MHz, CDCl₃) δ 7.89-7.73 (m, 3H), 7.49-7.42 (m, 1H), 7.37-7.27 (m, 3H), 7.16-7.08 (m, 3H), 5.10-5.01 (m, 1H), 4.99-4.73 (m, 2H), 4.58-4.50 (m, 1H), 4.48-4.39 (m, 1H), 4.35-4.26 (m, 1H), 4.22-4.05 (m, 2H), 4.00-3.91 (m, 1H), 3.74-3.53 (m, 7H), 3.45 (d, J=20.8 Hz, 2H), 3.33-3.23 (m, 2H), 3.18-3.08 (m, 1H), 2.37-2.24 (m, 3H), 2.16-1.97 (m, 4H), 1.67-1.61 (m, 1H), 1.50-1.38 (m, 1H), 1.21-1.16 (m, 6H), 1.12 (d, J=7.1 Hz, 3H), 1.01-0.91 (m, 1H), 0.86-0.77 (m, 1H), 0.06-0.00 (m, 1H) and Peak 2: Isopropyl ((S)-((2-(((3S,6S,7aS,8aR,9aR)-3-(3-(morpholine-4-carbonyl)azetidine-1-carbonyl)-5-oxodecahydro-1H-cyclopropa[d]pyrrolo[1,2-a]azocin-6-yl)carbamoyl)benzo[b]thiophen-5-yl)methyl)(phenoxy)phosphoryl)-L-alaninate or isopropyl ((R)-((2-(((3S,6S,7aS,8aR,9aR)-3-(3-(morpholine-4-carbonyl)azetidine-1-carbonyl)-5-oxodecahydro-1H-cyclopropa[d]pyrrolo[1,2-a]azocin-6-yl)carbamoyl)benzo[b]thiophen-5-yl)methyl)(phenoxy)phosphoryl)-L-alaninate (30.0 mg, 36.0 μmol, 99.7% ee, 50% yield) as a white solid. LCMS (ESI): m/z=834.4 [M+H]⁺; Peak 2 data (Example 6), P-chirality arbitrarily assigned: ¹H NMR (400 MHz, CDCl₃) δ 7.86-7.69 (m, 3H), 7.55-7.33 (m, 2H), 7.32-7.27 (m, 2H), 7.19-7.06 (m, 3H), 5.11-5.00 (m, 1H), 4.95-4.72 (m, 2H), 4.58-4.48 (m, 1H), 4.46-4.37 (m, 1H), 4.35-4.26 (m, 1H), 4.22-4.05 (m, 2H), 3.98-3.87 (m, 1H), 3.74-3.51 (m, 7H), 3.46-3.20 (m, 5H), 2.37-2.23 (m, 3H), 2.15-1.96 (m, 4H), 1.72-1.62 (m, 1H), 1.50-1.39 (m, 1H), 1.21-1.08 (m, 9H), 1.00-0.89 (m, 1H), 0.86-0.76 (m, 1H), 0.04-0.00 (m, 1H).

Synthesis of (difluoro(2-(((3S,6S,7aS,8aR,9aR)-5-oxo-3-(4-((R)-tetrahydrofuran-3-carbonyl)piperazine-1-carbonyl)decahydro-1H-cyclopropa[d]pyrrolo[1,2-a]azocin-6-yl)carbamoyl)benzo[b]thiophen-5-yl)methyl)phosphonic acid (Example 7)

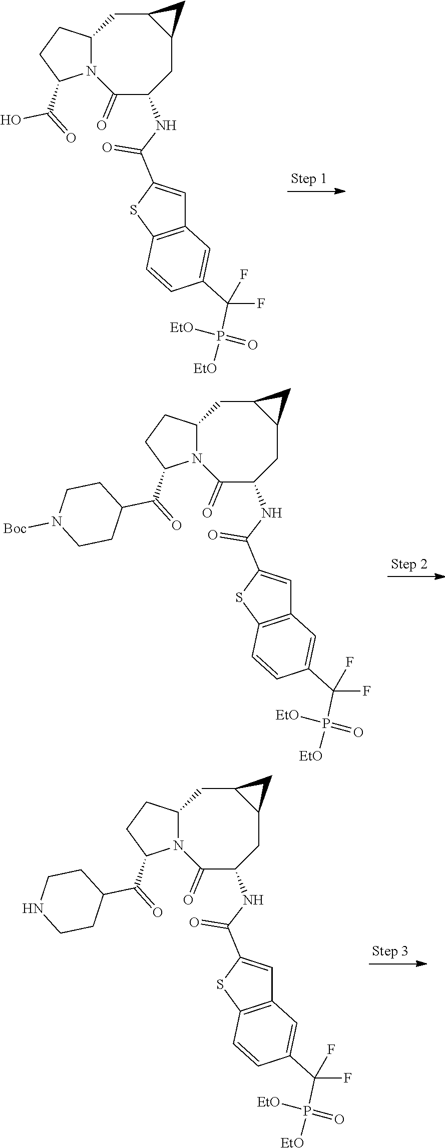

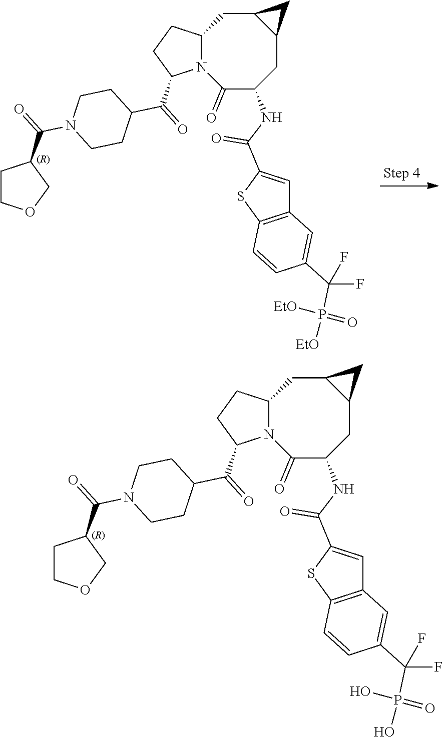

Step 1: Preparation of tert-butyl 4-((3S,6S,7aS,8aR,9aR)-6-(5-((diethoxyphosphoryl)difluoromethyl)benzo[b]thiophene-2-carboxamido)-5-oxodecahydro-1H-cyclopropa[d]pyrrolo[1,2-a]azocine-3-carbonyl)piperazine-1-carboxylate

To a solution of (3S,6S,7aS,8aR,9aR)-6-(5-((diethoxyphosphoryl)difluoromethyl)benzo[b]thiophene-2-carboxamido)-5-oxodecahydro-1H-cyclopropa[d]pyrrolo[1,2-a]azocine-3-carboxylic acid (0.4 g, 0.68 mmol, 1.0 eq) in DCM (1 mL) was added tert-butyl piperazine-1-carboxylate (0.13 g, 0.68 mmol, 1.0 eq), T₃P (0.46 g, 1.0 mmol, 1.5 eq, 50% purity) and DIEA (0.13 g, 1.0 mmol, 1.5 eq). The mixture was stirred at 20° C. for 16 h to give a yellow turbid solution. The reaction mixture was concentrated under reduced pressure to give a residue. The residue was purified by flash silica gel chromatography to give tert-butyl 4-((3S,6S,7aS,8aR,9aR)-6-(5-((diethoxyphosphoryl)difluoromethyl)benzo[b]thiophene-2-carboxamido)-5-oxodecahydro-1H-cyclopropa[d]pyrrolo[1,2-a]azocine-3-carbonyl)piperazine-1-carboxylate (0.43 g, 83% yield) as a white solid. LCMS: (ESI) m/z [M-Boc]+=653.2

Step 2: Preparation of diethyl (difluoro(2-(((3S,6S,7aS,8aR,9aR)-5-oxo-3-(piperazine-1-carbonyl)decahydro-1H-cyclopropa[d]pyrrolo[1,2-a]azocin-6-yl)carbamoyl)benzo[b]thiophen-5-yl)methyl)phosphonate

To a solution of tert-butyl 4-((3S,6S,7aS,8aR,9aR)-6-(5-((diethoxyphosphoryl) difluoromethyl)benzo[b]thiophene-2-carboxamido)-5-oxodecahydro-1H-cyclopropa[d]pyrrolo[1,2-a]azocine-3-carbonyl) piperazine-1-carboxylate (0.43 g, 0.56 mmol, 1.0 eq) in DCM (3.0 mL) was added TFA (1.0 mL). The resulting mixture was stirred at 25° C. for 1 h to give a yellow solution. The reaction mixture was concentrated under reduced pressure to give diethyl (difluoro(2-(((3S,6S,7aS,8aR,9aR)-5-oxo-3-(piperazine-1-carbonyl)decahydro-1H-cyclopropa[d]pyrrolo[1,2-a]azocin-6-yl)carbamoyl)benzo[b]thiophen-5-yl)methyl)phosphonate (0.43 g, crude) as a yellow oil.

Step 3: Preparation of diethyl (difluoro(2-(((3S,6S,7aS,8aR,9aR)-5-oxo-3-(4-((R)-tetrahydrofuran-3-carbonyl) piperazine-1-carbonyl)decahydro-1H-cyclopropa[d]pyrrolo[1,2-a]azocin-6-yl)carbamoyl)benzo[b]thiophen-5-yl)methyl)phosphonate

To a solution of diethyl (difluoro(2-(((3S,6S,7aS,8aR,9aR)-5-oxo-3-(piperazine-1-carbonyl)decahydro-1H-cyclopropa[d]pyrrolo[1,2-a]azocin-6-yl)carbamoyl)benzo[b]thiophen-5-yl)methyl)phosphonate (50 mg, 77 mol, 1.0 eq) in DMF (1.0 mL) was added DIEA (30 mg, 0.23 mmol, 3.0 eq), and the reaction was stirred at 20° C. for 30 minutes to give a colorless solution. A separate solution of (3R)-oxolane-3-carboxylic acid (8.9 mg, 77 mol, 1.0 eq) in DMF (1.0 mL) was added DIEA (30 mg, 0.23 mmol, 3.0 eq) and HATU (43 mg, 0.1 mmol, 1.5 eq) was stirred at 20° C. for 30 minutes to give a black solution. The two mixtures were combined and stirred at 20° C. for 1 h to give a solution. The mixture was quenched by water (10 mL) and extracted with EtOAc (10 mL×2), the combined organic layers were washed with saturated brine (10 mL×2), dried over Na₂SO₄, filtered and concentrated under reduced pressure to give diethyl (difluoro(2-(((3S,6S,7aS,8aR,9aR)-5-oxo-3-(4-((R)-tetrahydrofuran-3-carbonyl)piperazine-1-carbonyl)decahydro-1H-cyclopropa[d]pyrrolo[1,2-a]azocin-6-yl)carbamoyl)benzo[b]thiophen-5-yl)methyl)phosphonate (50 mg, crude) as a yellow oil. LCMS: (ESI) m/z [M+H]⁺=751.3

Step 4: Preparation of (difluoro(2-(((3S,6S,7aS,8aR,9aR)-5-oxo-3-(4-((R)-tetrahydrofuran-3-carbonyl)piperazine-1-carbonyl)decahydro-1H-cyclopropa[d]pyrrolo[1,2-a]azocin-6-yl)carbamoyl)benzo[b]thiophen-5-yl)methyl)phosphonic acid

To a solution of diethyl (difluoro(2-(((3S,6S,7aS,8aR,9aR)-5-oxo-3-(4-((R)-tetrahydrofuran-3-carbonyl)piperazine-1-carbonyl)decahydro-1H-cyclopropa[d]pyrrolo[1,2-a]azocin-6-yl)carbamoyl) benzo[b]thiophen-5-yl)methyl)phosphonate (50 mg, 0.07 mmol, 1.0 eq) in DCM (1.0 mL) was added BSTFA (0.1 g, 0.4 mmol, 6.0 eq) at 0° C., then trimethylsilyl iodide (54 mg, 0.27 mmol, 4.0 eq) was added dropwise and stirred at 0° C. for 0.5 h to give a yellow mixture. The reaction mixture was concentrated under reduced pressure to give a residue. The residue was purified by prep-HPLC (neutral; (column: Waters Xbridge 150×25 mm 10 um, mobile phase: water (10 mM NH₄HCO₃)-ACN; B %: 5%-35%, 10 min) follow by lyophilization to give (difluoro(2-(((3S,6S,7aS,8aR,9aR)-5-oxo-3-(4-((R)-tetrahydrofuran-3-carbonyl)piperazine-1-carbonyl)decahydro-1H-cyclopropa[d]pyrrolo[1,2-a]azocin-6-yl)carbamoyl)benzo[b]thiophen-5-yl)methyl)phosphonic acid (1.2 mg, 2.6% yield) as a white solid. LCMS: (ESI) m/z [M+H]⁺=695.2; ¹H NMR (400 MHz, CD₃OD) δ 8.17 (s, 1H), 8.11 (s, 1H), 7.93 (d, J=8.4 Hz, 1H), 7.73 (d, J=8.4 Hz, 1H), 5.09-5.01 (m, 1H), 5.00-4.95 (m, 1H), 4.31 (d, J=6.4 Hz, 1H), 3.95 (s, 1H), 3.91-3.65 (m, 8H), 3.64-3.51 (m, 2H), 3.50-3.40 (m, 2H), 2.53-2.42 (m, 1H), 2.33 (dd, J=2.0, 14.4 Hz, 1H), 2.19 (s, 5H), 2.05-1.87 (m, 2H), 1.85-1.68 (m, 1H), 1.45 (s, 1H), 1.02 (d, J=8.8 Hz, 1H), 0.91-0.79 (m, 1H), 0.08 (d, J=5.2 Hz, 1H)

(Difluoro(2-(((3S,6S,7aS,8aR,9aR)-5-oxo-3-(4-((S)-tetrahydrofuran-3-carbonyl)piperazine-1-carbonyl)decahydro-1H-cyclopropa[d]pyrrolo[1,2-a]azocin-6-yl)carbamoyl)benzo[b]thiophen-5-yl)methyl)phosphonic acid (Example 8) was prepared according to the procedure described above for (difluoro(2-(((3S,6S,7aS,8aR,9aR)-5-oxo-3-(4-((R)-tetrahydrofuran-3-carbonyl)piperazine-1-carbonyl)decahydro-1H-cyclopropa[d]pyrrolo[1,2-a]azocin-6-yl)carbamoyl)benzo[b]thiophen-5-yl)methyl)phosphonic acid using the appropriate starting materials:

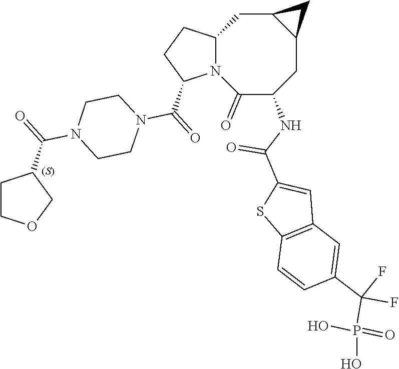

(Difluoro(2-(((3S,6S,7aS,8aR,9aR)-5-oxo-3-(4-((S)-tetrahydrofuran-3-carbonyl)piperazine-1-carbonyl)decahydro-1H-cyclopropa[d]pyrrolo[1,2-a]azocin-6-yl)carbamoyl)benzo[b]thiophen-5-yl)methyl)phosphonic acid: ¹H NMR (400 MHz, CD₃OD) δ 8.27-8.07 (m, 2H), 7.93 (d, J=8.4 Hz, 1H), 7.73 (d, J=8.8 Hz, 1H), 5.13-4.94 (m, 2H), 4.36-4.19 (m, 1H), 4.04-3.38 (m, 13H), 2.56-2.42 (m, 1H), 2.38-2.29 (m, 1H), 2.24-1.88 (m, 7H), 1.78-1.75 (m, 1H), 1.51-1.36 (m, 1H), 1.08-0.97 (m, 1H), 1.11-0.87 (m, 1H), 0.12-0.08 (m, 1H).

Synthesis of ((2-(((3S,6S,7aS,8aR,9aR)-3-(((3S,4S)-1-acetyl-4-hydroxypyrrolidin-3-yl)carbamoyl)-5-oxodecahydro-1H-cyclopropa[d]pyrrolo[1,2-a]azocin-6-yl)carbamoyl)benzo[b]thiophen-5-yl)difluoromethyl)phosphonic acid (Example 9)

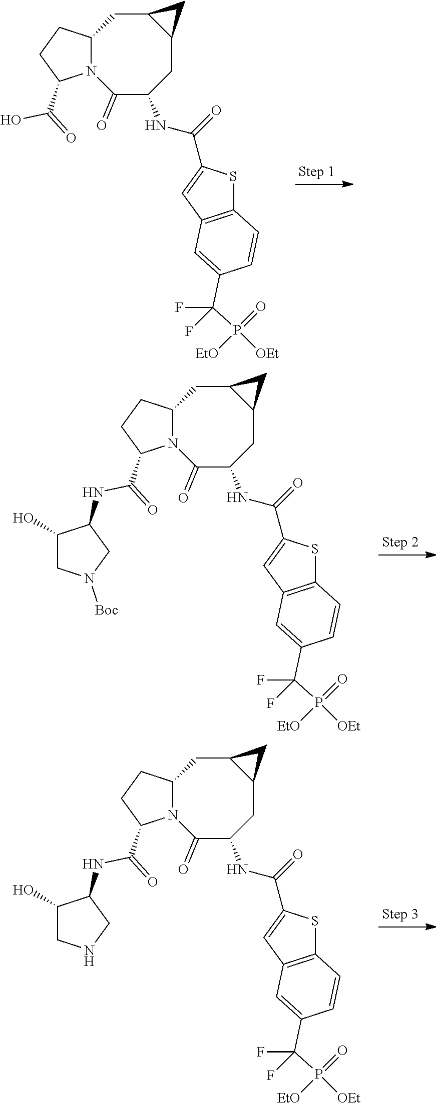

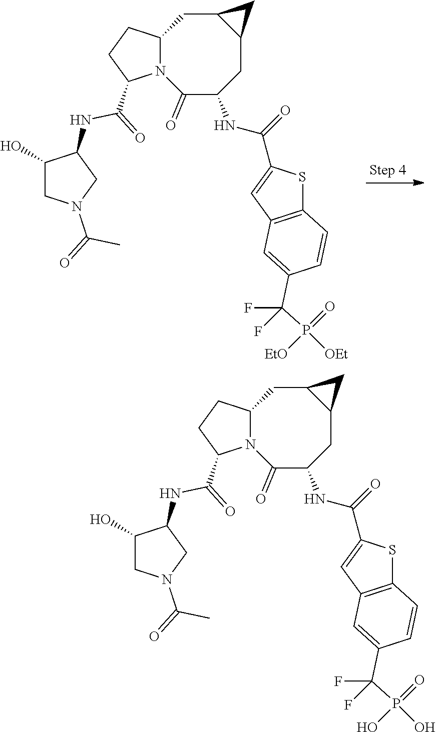

Step 1: Preparation of tert-butyl (3S,4S)-3-((3S,6S,7aS,8aR,9aR)-6-(5-((diethoxyphosphoryl)difluoromethyl) benzo[b]thiophene-2-carboxamido)-5-oxodecahydro-1H-cyclopropa[d]pyrrolo[1,2-a]azocine-3-carboxamido)-4-hydroxypyrrolidine-1-carboxylate

To solution of (3S,6S,7aS,8aR,9aR)-6-(5-((diethoxyphosphoryl)difluoromethyl)benzo[b]thiophene-2-carboxamido)-5-oxodecahydro-1H-cyclopropa[d]pyrrolo[1,2-a]azocine-3-carboxylic acid (0.1 g, 0.17 mmol, 1 eq) in DCM (5 mL), CMPI (44 mg, 0.34 mmol, 2 eq) and TEA (52 mg, 0.51 mmol, 3 eq), the mixture was stirred at 20° C. for 1 h to give a yellow solution. The solution was quenched by water (10 mL) and extracted with EtOAc (10 mL×2), the combined organic layers were washed with saturated brine (10 mL×2), dried over Na₂SO₄, filtered, and concentrated under reduced pressure to give a residue. The residue was purified by flash silica gel chromatography to give tert-butyl (3S,4S)-3-((3S,6S,7aS,8aR,9aR)-6-(5-((diethoxyphosphoryl)difluoromethyl) benzo[b]thiophene-2-carboxamido)-5-oxodecahydro-1H-cyclopropa[d]pyrrolo[1,2-a]azocine-3-carboxamido)-4-hydroxypyrrolidine-1-carboxylate (90 mg, 79% yield) as a yellow solid. LCMS: (ESI) m/z [M+H]⁺=769.2

Step 2: Preparation of diethyl (difluoro(2-(((3S,6S,7aS,8aR,9aR)-3-(((3S,4S)-4-hydroxypyrrolidin-3-yl)carbamoyl)-5-oxodecahydro-1H-cyclopropa[d]pyrrolo[1,2-a]azocin-6-yl)carbamoyl)benzo[b]thiophen-5-yl)methyl) phosphonate

A solution of tert-butyl (3S,4S)-3-((3S,6S,7aS,8aR,9aR)-6-(5-((diethoxyphosphoryl)difluoromethyl) benzo[b]thiophene-2-carboxamido)-5-oxodecahydro-1H-cyclopropa[d]pyrrolo[1,2-a]azocine-3-carboxamido)-4-hydroxypyrrolidine-1-carboxylate (90 mg, 0.12 mmol, 1 eq) in DCM (3 mL) and TFA (0.6 mL) was stirred at 20° C. for 1 h to give a yellow solution. The mixture was concentrated in vacuum to give diethyl (difluoro(2-(((3S,6S,7aS,8aR,9aR)-3-(((3S,4S)-4-hydroxypyrrolidin-3-yl)carbamoyl)-5-oxodecahydro-1H-cyclopropa[d]pyrrolo[1,2-a]azocin-6-yl)carbamoyl)benzo[b]thiophen-5-yl)methyl) phosphonate (90 mg, crude) as a brown oil. LCMS: (ESI) m/z [M+H]⁺=669.2

Step 3: Preparation of diethyl ((2-(((3S,6S,7aS,8aR,9aR)-3-(((3S,4S)-1-acetyl-4-hydroxypyrrolidin-3-yl)carbamoyl)-5-oxodecahydro-1H-cyclopropa[d]pyrrolo[1,2-a]azocin-6-yl)carbamoyl)benzo[b]thiophen-5-yl)difluoromethyl)phosphonate

A solution of diethyl (difluoro(2-(((3S,6S,7aS,8aR,9aR)-3-(((3S,4S)-4-hydroxypyrrolidin-3-yl) carbamoyl)-5-oxodecahydro-1H-cyclopropa[d]pyrrolo[1,2-a]azocin-6-yl)carbamoyl)benzo[b]thiophen-5-yl)methyl) phosphonate (90 mg, 0.12 mmol, 1 eq) in DCM (5 mL) and triethylamine (24 mg, 0.24 mmol, 2 eq) was stirred at 20° C. for 10 min, then Ac₂O (15 mg, 0.14 mmol, 1.2 eq) was added dropwise at 0° C. and the mixture was stirred at 20° C. for 2 h to give a yellow solution. The mixture was concentrated in vacuum to give a yellow solid. The solid was purified by flash silica gel chromatography to give diethyl ((2-(((3S,6S,7aS,8aR,9aR)-3-(((3S,4S)-1-acetyl-4-hydroxypyrrolidin-3-yl)carbamoyl)-5-oxodecahydro-1H-cyclopropa[d]pyrrolo[1,2-a]azocin-6-yl)carbamoyl)benzo[b]thiophen-5-yl)difluoromethyl)phosphonate (47 mg, 55% yield) as a yellow solid. ¹H NMR (400 MHz, CDCl₃) δ 8.10 (s, 1H), 7.98-7.89 (m, 2H), 7.71 (d, J=3.2 Hz, 1H), 7.63 (d, J=8.4 Hz, 1H), 5.18-5.06 (m, 1H), 4.72-4.57 (m, 1H), 4.32-4.14 (m, 6H), 3.81 (d, J=5.6 Hz, 1H), 3.72 (q, J=7.2 Hz, 3H), 3.08-3.06 (m, 1H), 3.14 (dd, J=4.4, 7.2 Hz, 1H), 2.41-2.18 (m, 4H), 2.09-2.05 (m, 2H), 1.95 (s, 3H), 1.61 (dd, J=3.6, 7.6 Hz, 1H), 1.34-1.30 (m, 6H), 0.86-0.71 (m, 2H), 0.07 (dd, J=4.0, 7.6 Hz, 1H)

Step 4: Preparation of ((2-(((3S,6S,7aS,8aR,9aR)-3-(((3S,4S)-1-acetyl-4-hydroxypyrrolidin-3-yl)carbamoyl)-5-oxodecahydro-1H-cyclopropa[d]pyrrolo[1,2-a]azocin-6-yl)carbamoyl)benzo[b]thiophen-5-yl) difluoromethyl)phosphonic acid

To solution of diethyl ((2-(((3S,6S,7aS,8aR,9aR)-3-(((3S,4S)-1-acetyl-4-hydroxypyrrolidin-3-yl)carbamoyl)-5-oxodecahydro-1H-cyclopropa[d]pyrrolo[1,2-a]azocin-6-yl)carbamoyl)benzo[b]thiophen-5-yl)difluoromethyl)phosphonate (47 mg, 86 μmol, 1 eq) in DCM (3 mL) was added dropwise BSTFA (89 mg, 0.34 mmol, 4 eq) and trimethylsilyl iodide (69 mg, 0.34 mmol, 4 eq) at 0° C., the reaction mixture was stirred at 0° C. for 1 h to give a yellow suspension. The mixture was concentrated in vacuum to give a brown solid. The solid was purified by prep-HPLC (Column: Phenomenex Luna C18 100×30 mm×5 um, Condition: water (10 mM NH₄HCO₃)-ACN, Flow Rate (ml/min): 25 min) follow by lyophilization to give ((2-(((3S,6S,7aS,8aR,9aR)-3-(((3S,4S)-1-acetyl-4-hydroxypyrrolidin-3-yl)carbamoyl)-5-oxodecahydro-1H-cyclopropa[d]pyrrolo[1,2-a]azocin-6-yl)carbamoyl)benzo[b]thiophen-5-yl) difluoromethyl)phosphonic acid (11 mg, 20% yield) as a yellow solid. LCMS: (ESI) m/z [M+H]⁺=655.1; ¹H NMR (400 MHz, CD₃OD) δ 8.19 (s, 1H), 8.13 (s, 1H), 7.93 (d, J=8.4 Hz, 1H), 7.74 (d, J=8.4 Hz, 1H), 5.03 (t, J=9.2 Hz, 1H), 4.61-4.50 (m, 1H), 4.31-4.25 (m, 1H), 4.19-4.05 (m, 2H), 3.89-3.53 (m, 2H), 3.49-3.36 (m, 2H), 2.54-2.40 (m, 1H), 2.34-2.25 (m, 1H), 2.23-2.09 (m, 3H), 2.05 (d, J=10.4 Hz, 4H), 2.01-1.92 (m, 1H), 1.81-1.62 (m, 1H), 1.25-0.99 (m, 1H), 0.93-0.81 (m, 2H), 0.10 (dd, J=3.2, 5.4 Hz, 1H)

### Phosphonate Acids Data Tables

### Prepared According to General Scheme 1: Phosphonate Acids

The following compounds in Table 26 were prepared according to the representative procedure described above for the synthesis of (difluoro(2-(((3S,6S,7aS,8aR,9aR)-3-(3-((R)-2-methylmorpholine-4-carbonyl)azetidine-1-carbonyl)-5-oxodecahydro-1H-cyclopropa[d]pyrrolo[1,2-a]azocin-6-yl)carbamoyl)benzo[b]thiophen-5-yl)methyl)phosphonic acid (Example 1) utilizing the appropriate starting materials and modifications.

TABLE 26

| Com- | | | | |
|---|---|---|---|---|
| pound | Name | Structure | LCMS | NMR |
| 10 | ((2- (((3S,6S,7aS, 8aR,9aR)-3- ((rel-trans)- 3-cyano-4- phenylpyrrolidine- 1- carbonyl)-5- oxodecahydro- 1H- cyclopropa[d] pyrrolo[1,2-a] azocin-6- yl)carbamoyl) benzo[b] thiophen-5- yl)difluoromethyl) phosphonic acid | 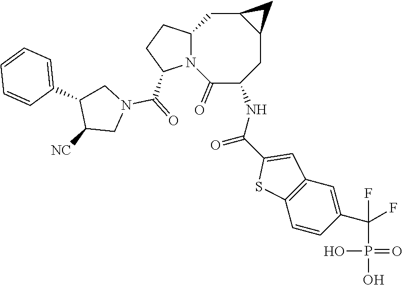 [SMILES: N#C[C@@H]1CN(C(=O)[C@@H]2CC[C@@H]3C[C@H]4C[C@H]4C[C@H](NC(=O)c4cc5cc(C(F)(F)P(=O)(O)O)ccc5s4)C(=O)N32)C[C@H]1c1ccccc1] | 683.3 [M + H]⁺ | ¹H NMR (400 MHz, DMSO-d₆) δ 8.93-8.82 (m, 1H), 8.38-8.29 (m, 1H), 8.15-8.02 (m, 2H), 7.61-7.53 (m, 1H), 7.50-7.27 (m, 5H), 5.00- 4.91 (m, 1H), 4.60-4.41 (m, 1H), 4.23-4.15 (m, 1H), 3.99-3.88 (m, 1H), 3.85-3.74 (m, 1H), 3.70- 3.61 (m, 1H), 3.57-3.48 (m, 1H), 3.25-3.06 (m, 1H), 2.41-2.30 (m, 1H), 2.22-1.99 (m, 4H), 1.95- 1.82 (m, 2H), 1.72-1.59 (m, 1H), 1.49-1.33 (m, 1H), 1.25-1.10 (m, 1H), 0.90-0.69 (m, 2H), 0.04- 0.00 (m, 1H). |
| 11 | ((2- (((3S,6S,7aR, 8aS,9aR)-3- ((rel-trans)- 3-cyano-4- phenylpyrrolidine- 1-carbonyl)-5- oxodecahydro-1H- cyclopropa[d] pyrrolo[1,2-a] azocin-6-yl) carbamoyl) benzo[b]thiophen- 5-yl)difluoromethyl) phosphonic acid | 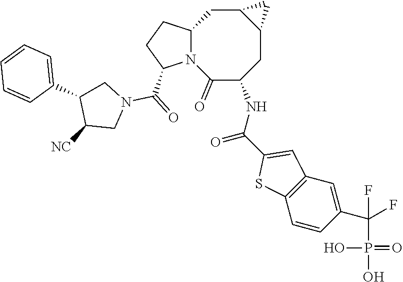 [SMILES: N#C[C@@H]1CN(C(=O)[C@@H]2CC[C@@H]3C[C@@H]4C[C@@H]4C[C@H](NC(=O)c4cc5cc(C(F)(F)P(=O)(O)O)ccc5s4)C(=O)N32)C[C@H]1c1ccccc1] | 683.3 [M + H]⁺ | ¹H NMR (400 MHz, DMSO-d₆) δ 9.00-8.74 (m, 1H), 8.44-8.28 (m, 1H), 8.26-8.00 (m, 2H), 7.75- 7.59 (m, 1H), 7.56-7.32 (m, 5H), 5.02-4.80 (m, 1H), 4.71-4.51 (m, 1H), 4.39-4.20 (m, 2H), 4.18- 3.86 (m, 4H), 3.35-3.24 (m, 1H), 2.42-2.26 (m, 2H), 2.16-1.88 (m, 4H), 1.79-1.60 (m, 2H), 1.44-1.22 (m, 2H), 0.92-0.74 (m, 1H), 0.21- 0.05 (m, 1H). |
| 12 | (difluoro(2- (((3S,6S,7aS,8aR, 9aR)-3-(3-((S)-2- methylmorpholine- 4-carbonyl) azetidine-1- carbonyl)-5- oxodecahydro-1H- cyclopropa[d] pyrrolo[1,2-a] azocin-6-yl) carbamoyl)benzo[b] thiophen-5-yl) methyl)phosphonic acid | 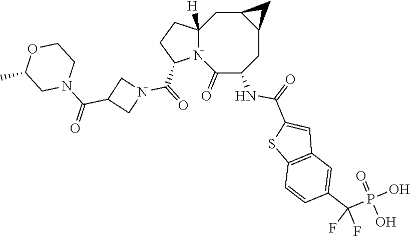 [SMILES: C[C@H]1CN(C(=O)C2CN(C(=O)[C@@H]3CC[C@@H]4C[C@H]5C[C@H]5C[C@H](NC(=O)c5cc6cc(C(F)(F)P(=O)(O)O)ccc6s5)C(=O)N43)C2)CCO1] | 695.3 [M + H]⁺ | ¹H NMR (400 MHz, DMSO-d₆) δ 8.92 (dd, J = 24.8, 7.4 Hz, 1H), 8.34 (s, 1H), 8.12 (d, J = 8.5 Hz, 1H), 8.07 (s, 1H), 7.58 (d, J = 8.7 Hz, 1H), 4.99-4.87 (m, 1H), 4.68- 4.43 (m, 1H), 4.39-4.24 (m, 2H), 4.22-4.09 (m, 2H), 4.06-3.85 (m, 2H), 3.81-3.64 (m, 2H), 3.45-3.31 (m, 3H), 3.15-2.98 (m, 1H), 2.77-2.63 (m, 1H), 2.38- 2.27 (m, 1H), 2.18-1.93 (m, 4H), 1.90-1.74 (m, 2H), 1.69-1.52 (m, 1H), 1.40-1.27 (m, 1H), 1.13-1.00 (m, 3H), 0.84-0.66 (m, 2H), 0.00- −0.05 (m, 1H). |
| 13 | (difluoro(2- (((3S,6S,7aS,8aR, 9aR)-3-(3-((R)-3- methylmorpholine- 4-carbonyl) azetidine-1-carbonyl) 5-oxodecahydro-1H- cyclopropa[d] pyrrolo[1,2-a]azocin- 6-yl)carbamoyl) benzo[b]thiophen-5- yl)methyl) phosphonic acid | 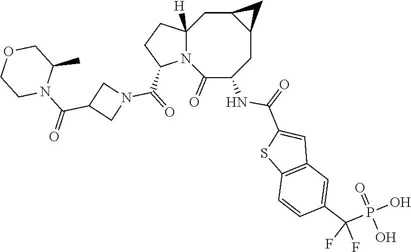 [SMILES: C[C@@H]1COCCN1C(=O)C1CN(C(=O)[C@@H]2CC[C@@H]3C[C@H]4C[C@H]4C[C@H](NC(=O)c4cc5cc(C(F)(F)P(=O)(O)O)ccc5s4)C(=O)N32)C1] | 695.3 [M + H]⁺ | ¹H NMR (400 MHz, DMSO-d₆) δ 8.96-8.81 (m, 1H), 8.39-8.10 (m, 3H), 7.74-7.57 (m, 1H), 4.99-4.90 (m, 1H), 4.73-4.49 (m, 2H), 4.38- 4.30 (m, 2H), 4.26-4.13 (m, 2H), 4.03-3.89 (m, 2H), 3.82-3.73 (m, 1H), 3.67-3.54 (m, 2H), 3.48- 3.40 (m, 1H), 3.31-3.18 (m, 2H), 2.39-2.27 (m, 1H), 2.18-1.94 (m, 4H), 1.90-1.73 (m, 2H), 1.70-1.57 (m, 1H), 1.40-1.28 (m, 1H), 1.25- 1.06 (m, 3H), 0.89-0.63 (m, 2H), 0.00-−0.03 (m, 1H). |
| 14 | (difluoro(2- (((3S,6S,7aS,8aR, 9aR)-3-(3-((S)-3- methylmorpholine- 4-carbonyl) azetidine-1- carbonyl)-5- oxodecahydro-1H- cyclopropa[d] pyrrolo[1,2-a]azocin- 6-yl)carbamoyl) benzo[b]thiophen-5- yl)methyl) phosphonic acid | 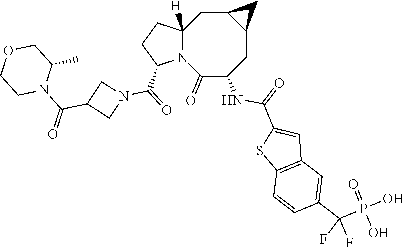 [SMILES: C[C@H]1COCCN1C(=O)C1CN(C(=O)[C@@H]2CC[C@@H]3C[C@H]4C[C@H]4C[C@H](NC(=O)c4cc5cc(C(F)(F)P(=O)(O)O)ccc5s4)C(=O)N32)C1] | 695.3 [M + H]⁺ | ¹H NMR (400 MHz, DMSO-d₆) δ 8.99-8.81 (m, 1H), 8.38-8.29 (m, 1H), 8.24-8.08 (m, 2H), 7.73- 7.57 (m, 1H), 4.98-4.89 (m, 1H), 4.68-4.56 (m, 1H), 4.52-4.40 (m, 1H), 4.37-4.29 (m, 2H), 4.20-4.14 (m, 1H), 4.08-3.92 (m, 2H), 3.83- 3.75 (m, 1H), 3.68-3.54 (m, 2H), 3.48-3.40 (m, 1H), 3.35-3.20 (m, 2H), 3.13-2.90 (m, 1H), 2.39- 2.26 (m, 1H), 2.20-1.92 (m, 4H), 1.90-1.75 (m, 2H), 1.69-1.56 (m, 1H), 1.41-1.28 (m, 1H), 1.25- 1.07 (m, 3H), 0.89-0.61 (m, 2H), 0.01-−0.01 (m, 1H). |
| 15 | (difluoro(2- (((3S,6S,7aS,8aR, 9aR)-3-(3- (morpholine-4- carbonyl)azetidine- 1-carbonyl)-5- oxodecahydro-1H- cyclopropa[d] pyrrolo[1,2-a]azocin- 6-yl)carbamoyl) benzo[b]thiophen-5- yl)methyl) phosphonic acid | 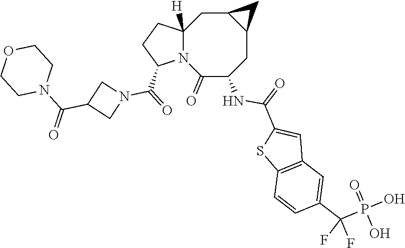 [SMILES: O=C(N[C@H]1C[C@@H]2C[C@@H]2C[C@H]2CC[C@@H](C(=O)N3CC(C(=O)N4CCOCC4)C3)N2C1=O)c1cc2cc(C(F)(F)P(=O)(O)O)ccc2s1] | 681 [M + H]⁺ | ¹H NMR (400 MHz, DMSO-d₆) δ 8.90 (m, 1H), 8.33 (s, 1H), 8.14 (d, J = 8.6 Hz, 1H), 8.08 (s, 1H), 7.59 (d, J = 8.5 Hz, 1H), 4.99-4.88 (m, 1H), 4.57 (dt, J = 14.7, 7.8 Hz, 1H), 4.37-4.25 (m, 2H), 4.20-4.13 (m, 1H), 4.03-3.93 (m, 2H), 3.72-3.71 (m, 1H), 3.53- 3.52 (m, 4H), 3.47-3.46 (m, 2H), 3.28-3.23 (m, 2H), 2.38-2.28 (m, 1H), 2.16-1.96 (m, 4H), 1.90-1.76 (m, 2H), 1.69-1.57 (m, 1H), 1.40- 1.30 (m, 1H), 0.84-0.68 (m, 2H), 0.03-−0.02 (m, 1H). |
| 16 | (difluoro(2- (((3S,6S,7aS,8aR, 9aR)-3-((R)-3- morpholino- pyrrolidine-1- carbonyl)-5- oxodecahydro-1H- cyclopropa[d] pyrrolo[1,2-a]azocin- 6-yl)carbamoyl) benzo[b]thiophen- 5-yl)methyl) phosphonic acid | 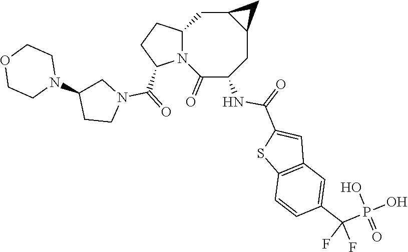 [SMILES: O=C(N[C@H]1C[C@@H]2C[C@@H]2C[C@H]2CC[C@@H](C(=O)N3CC[C@@H](N4CCOCC4)C3)N2C1=O)c1cc2cc(C(F)(F)P(=O)(O)O)ccc2s1] | 667.0 [M + H]⁺ | ¹H NMR (400 MHz, DMSO-d₆) δ 8.95-8.81 (m, 1H), 8.32-8.24 (m, 1H), 8.09-7.98 (m, 2H), 7.59 (d, J = 8.3 Hz, 1H), 5.00-4.90 (m, 1H), 4.63-4.56 (m, 1H), 4.24-4.17 (m, 1H), 4.01-3.92 (m, 1H), 3.79- 3.68 (m, 4H), 3.33-3.23 (m, 4H), 2.95-2.84 (m, 2H), 2.79-2.65 (m, 2H), 2.39-2.27 (m, 1H), 2.22-1.72 (m, 9H), 1.69-1.59 (m, 1H), 1.45- 1.35 (m, 1H), 0.89-0.65 (m, 2H). |
| 17 | (difluoro(2- (((3S,6S,7aS,8aR, 9aR)-3-((S)-3- morpholino- pyrrolidine-1- carbonyl)-5- oxodecahydro-1H- cyclopropa[d] pyrrolo[1,2-a] azocin-6-yl) carbamoyl)benzo[b] thiophen-5-yl) methyl)phosphonic acid | 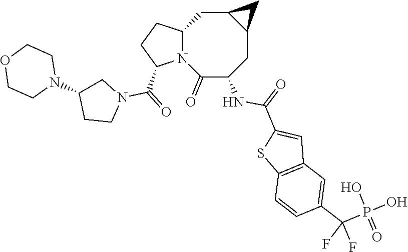 [SMILES: O=C(N[C@H]1C[C@@H]2C[C@@H]2C[C@H]2CC[C@@H](C(=O)N3CC[C@H](N4CCOCC4)C3)N2C1=O)c1cc2cc(C(F)(F)P(=O)(O)O)ccc2s1] | 667.0 [M + H]⁺ | ¹H NMR (400 MHz, DMSO-d₆) δ 8.88-8.74 (m, 1H), 8.34-8.22 (m, 1H), 8.16-7.99 (m, 2H), 7.59 (d, J = 8.7 Hz, 1H), 4.99-4.88 (m, 1H), 4.64-4.47 (m, 1H), 4.25-4.18 (m, 1H), 3.92-3.86 (m, 1H), 3.82- 3.75 (m, 1H), 3.74-3.68 (m, 4H), 3.56-3.51 (m, 1H), 3.42-3.30 (m, 1H), 3.23-3.10 (m, 1H), 2.93-2.75 (m, 4H), 2.43-2.27 (m, 1H), 2.24- 1.90 (m, 6H), 1.94-1.73 (m, 3H), 1.71-1.55 (m, 1H), 1.50-1.34 (m, 1H), 0.87-0.65 (m, 2H). |
| 18 | (fluoro(2- (((3S,6S,7aS,8aR, 9aR)-3-(3- (morpholine-4- carbonyl)azetidine- 1-carbonyl)-5- oxodecahydro-1H- cyclopropa[d] pyrrolo[1,2-a]azocin- 6-yl)carbamoyl) benzo[b]thiophen-5- yl)methyl) phosphonic acid | 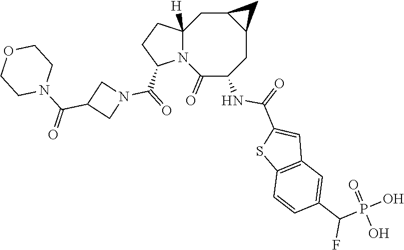 [SMILES: O=C(N[C@H]1C[C@@H]2C[C@@H]2C[C@H]2CC[C@@H](C(=O)N3CC(C(=O)N4CCOCC4)C3)N2C1=O)c1cc2cc(C(F)P(=O)(O)O)ccc2s1] | 663.0 [M + H]⁺ | ¹H NMR (400 MHz, DMSO-d₆) δ 8.84 (dd, J = 24.2, 7.4 Hz, 1H), 8.29 (s, 1H), 8.03 (d, J = 8.5 Hz, 1H), 7.98 (s, 1H), 7.53 (d, J = 8.6 Hz, 1H), 5.82 (dd, J = 44.3, 8.0 Hz, 1H), 4.98-4.88 (m, 1H), 4.57 (dt, J = 14.7, 7.9 Hz, 1H), 4.40- 4.24 (m, 2H), 4.16 (s, 1H), 4.02- 3.86 (m, 2H), 3.72-3.64 (m, 1H), 3.56-3.49 (m, 4H), 3.48-3.42 (m, 2H), 3.29-3.22 (m, 2H), 2.38-2.24 (m, 1H), 2.17-1.93 (m, 4H), 1.90- 1.74 (m, 2H), 1.69-1.56 (m, 1H), 1.44-1.27 (m, 1H), 0.85-0.65 (m, 2H), 0.00-−0.04 (m, 1H). |
| 19 | ((2- (((3S,6S,7aS,8aR, 9aR)-3-((2R,3S)-3- (1H-imidazol-1-yl)- 2-methylazetidine- 1-carbonyl)-5- oxodecahydro-1H- cyclopropa[d] pyrrolo[1,2-a] azocin-6-yl) carbamoyl)benzo[b] thiophen-5-yl) difluoromethyl) phosphonic acid | 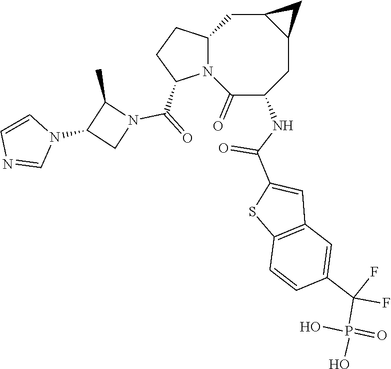 [SMILES: C[C@@H]1[C@@H](n2ccnc2)CN1C(=O)[C@@H]1CC[C@@H]2C[C@H]3C[C@H]3C[C@H](NC(=O)c3cc4cc(C(F)(F)P(=O)(O)O)ccc4s3)C(=O)N21] | 648.0 [M + H]⁺ | ¹H NMR (400 MHz, DMSO-d₆) δ 9.07-8.75 (m, 2H), 8.35 (s, 1H), 8.15-8.06 (m, 2H), 7.79 (s, 1H), 7.60 (d, J = 8.6 Hz, 1H), 7.47-7.17 (m, 1H), 5.01-4.81 (m, 2H), 4.77- 4.64 (m, 2H), 4.47-4.36 (m, 1H), 4.31-4.18 (m, 2H), 2.17-2.09 (m, 2H), 2.06-1.96 (m, 3H), 1.92-1.82 (m, 2H), 1.69-1.63 (m, 1H), 1.45 (d, J = 6.2 Hz, 3H), 1.35-1.29 (m, 1H), 0.85-0.79 (m, 1H), 0.77-0.69 (m, 1H), 0.05-0.00 (m, 1H). |
| 20 | ((2- (((3S,7aS,8aR, 9aR)-3-(3- (morpholine-4- carbonyl)azetidine- 1-carbonyl)-5- oxodecahydro-1H- cyclopropa[d] pyrrolo[1,2-a]azocin- 6-yl)carbamoyl) benzo[b]thiophen- 5-yl)methyl) phosphonic acid | 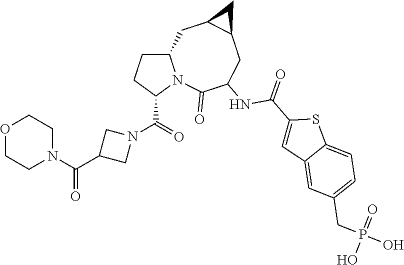 | 645.2 [M + H]⁺ | ¹H NMR (400 MHz, DMSO-d₆) δ 8.79 (dd, J = 23.8, 7.5 Hz, 1H), 8.21 (s, 1H), 7.91 (d, J = 8.3 Hz, 1H), 7.79 (s, 1H), 7.43-7.29 (m, 1H), 5.02-4.88 (m, 1H), 4.64-4.51 (m, 1H), 4.35-4.26 (m, 2H), 4.19-4.11 (m, 1H), 4.06-3.83 (m, 3H), 3.75-3.64 (m, 1H), 3.59-3.49 (m, 4H), 3.45 (d, J = 10.1 Hz, 2H), 3.29-3.21 (m, 2H), 3.10 (d, J = 21.1 Hz, 2H), 2.36-2.23 (m, 1H), 2.16-1.91 (m, 4H), 1.88-1.73 (m, 2H), 1.69-1.55 (m, 1H), 1.41- 1.26 (m, 1H), 0.86-0.63 (m, 2H). |
| 21 | ((R)- fluoro(2- (((3S,6S,7aR, 8aS,9aR)-5- oxo-3-(3- (pyridin-3- yl)azetidine-1- carbonyl) decahydro-1H- cyclopropa[d] pyrrolo[1,2-a] azocin-6-yl) carbamoyl)benzo[b] thiophen-5-yl) methyl) phosphonic acid | 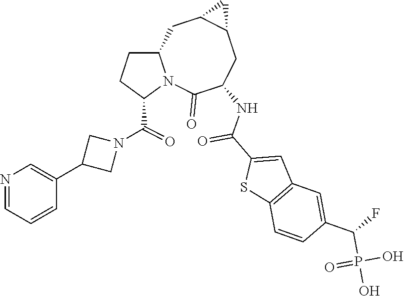 [SMILES: O=C(N[C@H]1C[C@H]2C[C@H]2C[C@H]2CC[C@@H](C(=O)N3CC(c4cccnc4)C3)N2C1=O)c1cc2cc([C@H](F)P(=O)(O)O)ccc2s1] | 627.1 [M + H]⁺ | ¹H NMR (400 MHz, DMSO-d₆) δ 8.87 (dd, J = 23.3, 7.5 Hz, 1H), 8.57 (s, 1H), 8.51-8.43 (m, 1H), 8.28 (d, J = 2.3 Hz, 1H), 8.04 (d, J = 8.4 Hz, 1H), 8.00 (s, 1H), 7.90-7.82 (m, 1H), 7.53 (d, J = 8.5 Hz, 1H), 7.42-7.39 (m, 1H), 5.82 (dd, J = 44.4, 8.0 Hz, 1H), 4.85- 4.81 (m, 1H), 4.78 (t, 0.5H), 4.61 (t, 0.5H), 4.43-4.30 (m, 2H), 4.25- 4.17 (m, 2H), 3.96-3.90 (m, 1H), 3.87-3.83 (m, 1H), 2.31-2.14 (m, 2H), 2.12-1.97 (m, 2H), 1.96-1.81 (m, 2H), 1.76-1.61 (m, 1H), 1.59- 1.44 (m, 1H), 1.39-1.27 (m, 1H), 1.26-1.14 (m, 1H), 0.83-0.70 (m, 1H), 0.09-0.01 (m, 1H). |
| 22 | ((2- (((3S,6S,7aR,8aS, 9aR)-5-oxo-3-(3- (pyridin-3-yl) azetidine-1-carbonyl) decahydro-1H- cyclopropa[d] pyrrolo[1,2-a] azocin-6-yl) carbamoyl)benzo[b] thiophen-5- yl)methyl) phosphonic acid | 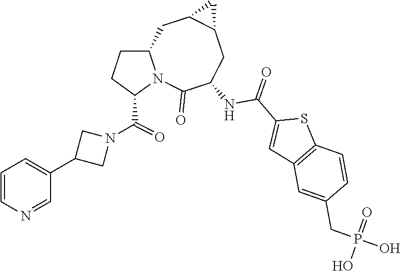 [SMILES: O=C(N[C@H]1C[C@H]2C[C@H]2C[C@H]2CC[C@@H](C(=O)N3CC(c4cccnc4)C3)N2C1=O)c1cc2cc(CP(=O)(O)O)ccc2s1] | 609.2 [M + H]⁺ | ¹H NMR (400 MHz, DMSO-d₆) δ 8.81 (dd, J = 23.0, 7.5 Hz, 1H), 8.56 (s, 1H), 8.50-8.44 (m, 1H), 8.20 (s, 1H), 7.91 (d, J = 8.3 Hz, 1H), 7.88-7.82 (m, 1H), 7.81 (s, 1H), 7.44-7.33 (m, 2H), 4.85-4.77 (m, 1H), 4.61 (t, J = 8.5 Hz, 1H), 4.41-4.38 (m, 1H), 4.33-4.29 (m, 1H), 4.25-4.16 (m, 2H), 3.96- 3.89 (m, 1H), 3.88-3.82 (m, 1H), 3.10 (d, J = 21.2 Hz, 2H), 2.30- 2.14 (m, 2H), 2.13-1.97 (m, 2H), 1.94-1.83 (m, 2H), 1.78-1.62 (m, 1H), 1.60-1.47 (m, 1H), 1.40-1.27 (m, 1H), 1.26-1.15 (m, 1H), 0.80- 0.75 (m, 1H), 0.07-0.03 (m, 1H). |
| 23 | ((2- (((3S,6S,7aR,8aS, 9aR)-5-oxo-3-(3- (pyridin-2-yl) azetidine-1- carbonyl)decahydro- 1H-cyclopropa[d] pyrrolo[1,2-a]azocin- 6-yl)carbamoyl) benzo[b]thiophen-5- yl)methyl) phosphonic acid | 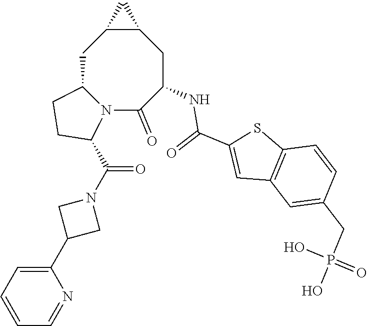 [SMILES: O=C(N[C@H]1C[C@H]2C[C@H]2C[C@H]2CC[C@@H](C(=O)N3CC(c4ccccn4)C3)N2C1=O)c1cc2cc(CP(=O)(O)O)ccc2s1] | 609.2 [M + H]⁺ | ¹H NMR (400 MHz, DMSO-d₆) δ 8.81-8.73 (m, 1H), 8.64-8.52 (m, 1H), 8.23-8.13 (m, 1H), 7.95- 7.87 (m, 1H), 7.84-7.68 (m, 2H), 7.45-7.19 (m, 3H), 4.82-4.70 (m, 1H), 4.55-4.47 (m, 1H), 4.38-4.28 (m, 2H), 4.23-4.15 (m, 2H), 4.05- 3.96 (m, 2H), 3.13-3.06 (m, 2H), 2.30-2.12 (m, 2H), 2.09-1.80 (m, 4H), 1.75-1.47 (m, 2H), 1.40-1.14 (m, 2H), 0.81-0.71 (m, 1H), 0.09- 0.01 (m, 1H). |
| 24 | ((2- (((3S,6S,7aS,8aR, 9aR)-3-(3-(1H- imidazol-1-yl) azetidine-1- carbonyl)-5- oxodecahydro-1H- cyclopropa[d] pyrrolo[1,2-a]azocin- 6- yl)carbamoyl) benzo[b]thiophen- 5-yl)methyl) phosphonic acid | 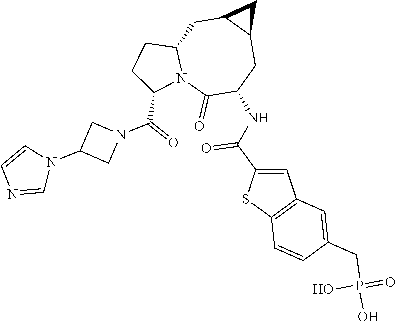 [SMILES: O=C(N[C@H]1C[C@@H]2C[C@@H]2C[C@H]2CC[C@@H](C(=O)N3CC(n4ccnc4)C3)N2C1=O)c1cc2cc(CP(=O)(O)O)ccc2s1] | 598.1 [M + H]⁺ | ¹H NMR (400 MHz, DMSO-d₆) δ 9.11-8.89 (m, 1H), 8.86-8.73 (m, 1H), 8.26-8.05 (m, 1H), 7.99-7.71 (m, 3H), 7.65-7.44 (m, 1H), 7.44- 7.34 (m, 1H), 5.39-5.20 (m, 1H), 5.03-4.92 (m, 1H), 4.79-4.67 (m, 1H), 4.48-4.30 (m, 3H), 4.21- 4.11 (m, 2H), 3.14-3.08 (m, 2H), 2.38-2.32 (m, 1H), 2.20-1.83 (m, 6H), 1.73-1.58 (m, 1H), 0.87- 0.65 (m, 2H), 0.05-0.00 (m, 1H). |
| 25 | ((2- (((3S,6S,7aR,8aS, 9aR)-3-(3-(1H- imidazol-1-yl) azetidine-1- carbonyl)-5- oxodecahydro-1H- cyclopropa[d] pyrrolo[1,2-a] azocin-6-yl) carbamoyl)benzo[b] thiophen-5-yl) methyl)phosphonic acid | 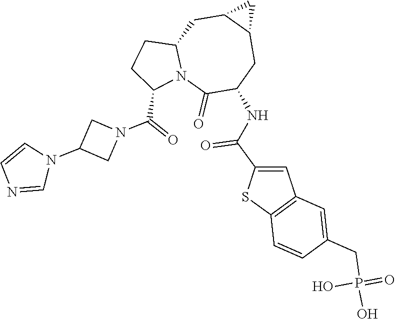 [SMILES: O=C(N[C@H]1C[C@H]2C[C@H]2C[C@H]2CC[C@@H](C(=O)N3CC(n4ccnc4)C3)N2C1=O)c1cc2cc(CP(=O)(O)O)ccc2s1] | 598.2 [M + H]⁺ | ¹H NMR (400 MHz, DMSO-d₆) δ 8.88-8.74 (m, 1H), 8.24-8.13 (m, 1H), 7.98-7.74 (m, 3H), 7.54-7.28 (m, 2H), 7.06-6.90 (m, 1H), 5.26- 5.10 (m, 1H), 4.85-4.80 (m, 1H), 4.72-4.50 (m, 1H), 4.37-4.28 (m, 2H), 4.22-4.14 (m, 1H), 4.07-3.89 (m, 2H), 3.12-3.05 (m, 2H), 2.30- 2.15 (m, 2H), 2.11-1.99 (m, 2H), 1.95-1.83 (m, 2H), 1.77-1.64 (m, 1H), 1.57-1.45 (m, 1H), 1.36-1.17 (m, 2H), 0.82-0.72 (m, 1H), 0.11- 0.02 (m, 1H). |
| 26 | ((2- (((3S,6S,7aS,8aR, 9aR)-3-((3-(2- amino-2-oxoethyl)- [1,1′-biphenyl]-4- yl)carbamoyl)-5- oxodecahydro-1H- cyclopropa[d] pyrrolo[1,2-a] azocin-6-yl) carbamoyl)benzo[b] thiophen-5-yl) difluoromethyl) phosphonic acid | 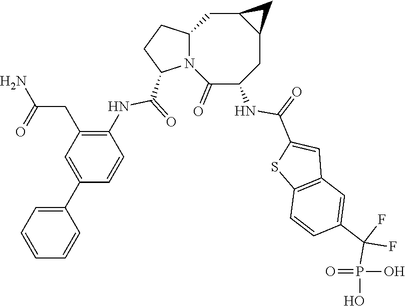 [SMILES: NC(=O)Cc1cc(-c2ccccc2)ccc1NC(=O)[C@@H]1CC[C@@H]2C[C@H]3C[C@H]3C[C@H](NC(=O)c3cc4cc(C(F)(F)P(=O)(O)O)ccc4s3)C(=O)N21] | 737.2 [M + H]⁺ | ¹H NMR (400 MHz, CD₃OD) δ 8.20-8.12 (m, 2H), 8.02 (d, J = 8.6 Hz, 1H), 7.67 (d, J = 8.6 Hz, 1H), 7.63-7.55 (m, 4H), 7.55-7.49 (m, 1H), 7.42 (t, J = 7.6 Hz, 2H), 7.36- 7.28 (m, 1H), 5.10 (t, J = 9.0 Hz, 1H), 4.79 (d, J = 7.3 Hz, 1H), 4.40- 4.30 (m, 1H), 3.78-3.69 (m, 1H), 3.67-3.56 (m, 1H), 2.54-2.41 (m, 1H), 2.36-2.13 (m, 5H), 2.06-1.91 (m, 1H), 1.81-1.67 (m, 1H), 1.28- 1.16 (m, 1H), 1.01-0.88 (m, 1H), 0.87-0.77 (m, 1H), 0.11-0.01 (m 1H). |
| 27 | ((2-(((3S,6S,7aS, 8aR,9aR)-3-((3R, 4′R)-4′-cyano-2- oxospiro[indoline- 3,3′-pyrrolidine]-1′- carbonyl)-5-oxo- decahydro-1H-cyclo- propa[d]pyrrolo[1,2- a]azocin-6-yl) carbamoyl)benzo[b] thiophen-5-yl) difluoromethyl) phosphonic acid Or ((2-(((3S,6S,7aS, 8aR,9aR)-3-((3S, 4′S)-4′-cyano-2- oxospiro[indoline-3, 3′-pyrrolidin]-1′- yl)carbonyl)-5-oxo- decahydro-1H- cyclopropa[d] pyrrolo[1,2-a]azocin- 6-yl)carbamoyl) benzo[b]thiophen- 5-yl)difluoro- methyl)phosphonic acid | 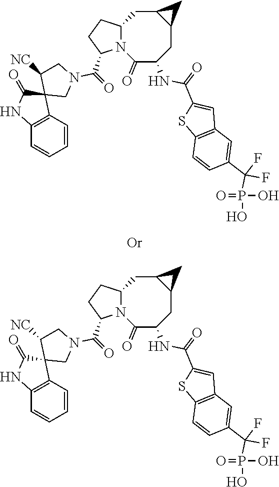 [SMILES: N#C[C@H]1CN(C(=O)[C@@H]2CC[C@@H]3C[C@H]4C[C@H]4C[C@H](NC(=O)c4cc5cc(C(F)(F)P(=O)(O)O)ccc5s4)C(=O)N32)C[C@@]12C(=O)Nc1ccccc12 ; N#C[C@@H]1CN(C(=O)[C@@H]2CC[C@@H]3C[C@H]4C[C@H]4C[C@H](NC(=O)c4cc5cc(C(F)(F)P(=O)(O)O)ccc5s4)C(=O)N32)C[C@]12C(=O)Nc1ccccc12] | 724.2 [M + H]⁺ | ¹H NMR (400 MHz, CD₃OD) δ 8.22-8.13 (m, 2H), 8.05-7.98 (m, 1H), 7.70-7.59 (m, 2H), 7.38-7.27 (m, 1H), 7.12 (q, J = 7.0 Hz, 1H), 7.03-6.97 (m, 1H), 5.13-5.02 (m, 1H), 4.85-4.82 (m, 1H), 4.69-4.47 (m, 1H), 4.44-4.15 (m, 2H), 3.98- 3.80 (m, 2H), 3.66 (d, J = 12.0 Hz, 1H), 2.54-2.42 (m, 1H), 2.39-1.86 (m, 6H), 1.82-1.70 (m, 1H), 1.54- 1.26 (m, 1H), 1.06-0.78 (m, 2H), 0.12-0.02 (m, 1H). |
| | | SFC Peak 1 was used for | | |
| | | biological testing and/or further | | |
| | | synthesis | | |
| 28 | ((2-(((3S,6S,7aS, 8aR,9aR)-3-((3S, 4′S)-4′-cyano-2- oxospiro[indoline- 3,3′-pyrrolidin]-1′- ylcarbonyl)-5-oxo- decahydro-1H- cyclopropa[d] pyrrolo[1,2-a]azocin- 6-yl)carbamoyl) benzo[b]thiophen-5- yl)difluoromethyl) phosphonic acid Or ((2-(((3S,6S,7aS, 8aR,9aR)-3-((3S, 4′S)-4′-cyano-2- oxospiro[indoline- 3,3′-pyrrolidin]-1′- ylcarbonyl)-5-oxo- decahydro-1H- cyclopropa[d] pyrrolo[1,2-a]azocin- 6-yl)carbamoyl) benzo[b]thiophen-5- yl)difluoromethyl) phosphonic acid | 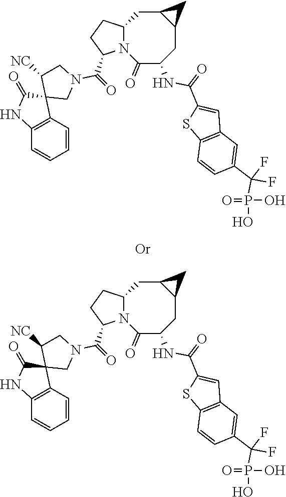 [SMILES: N#C[C@@H]1CN(C(=O)[C@@H]2CC[C@@H]3C[C@H]4C[C@H]4C[C@H](NC(=O)c4cc5cc(C(F)(F)P(=O)(O)O)ccc5s4)C(=O)N32)C[C@]12C(=O)Nc1ccccc12 ; N#C[C@H]1CN(C(=O)[C@@H]2CC[C@@H]3C[C@H]4C[C@H]4C[C@H](NC(=O)c4cc5cc(C(F)(F)P(=O)(O)O)ccc5s4)C(=O)N32)C[C@@]12C(=O)Nc1ccccc12] | 724.2 [M + H]⁺ | ¹H NMR (400 MHz, CD₃OD) δ 8.19-8.08 (m, 2H), 8.04-7.95 (m, 1H), 7.66 (t, J = 8.1 Hz, 1H), 7.45- 7.27 (m, 2H), 7.16-7.05 (m, 1H), 7.04-6.93 (m, 1H), 5.11-5.00 (m, 1H), 4.84-4.68 (m, 1H), 4.38- 4.13 (m, 3H), 3.99-3.69 (m, 3H), 2.53-2.41 (m, 1H), 2.38-1.94 (m, 6H), 1.82-1.71 (m, 1H), 1.53-1.35 (m, 1H), 1.10-0.81 (m, 2H), 0.11-0.05 (m, 1H). |
| | | SFC Peak 2 was used for | | |
| | | biological testing and/or further | | |
| | | synthesis | | |
| 29 | ((2- (((3S,6S,7aS,8aR, 9aR)-8,8-difluoro- 3-(3-methoxy-3- (1-methyl-1H- imidazol-2-yl) azetidine-1- carbonyl)-5- oxodecahydro-1H- cyclopropa[d] pyrrolo[1,2-a]azocin- 6-yl)carbamoyl) benzo[b]thiophen-5- yl)difluoromethyl) phosphonic acid | 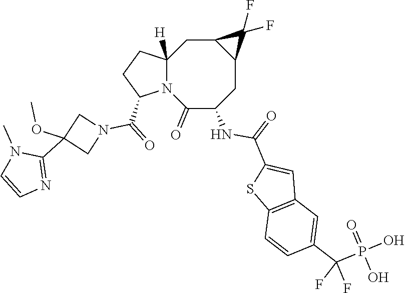 [SMILES: COC1(c2nccn2C)CN(C(=O)[C@@H]2CC[C@@H]3C[C@@H]4[C@H](C[C@H](NC(=O)c5cc6cc(C(F)(F)P(=O)(O)O)ccc6s5)C(=O)N32)C4(F)F)C1] | 714.0 [M + H]⁺ | ¹H NMR (400 MHz, DMSO-d₆: D₂O) δ 8.29-8.19 (m, 1H), 8.09- 7.98 (m, 2H), 7.64-7.57 (m, 1H), 7.26-7.18 (m, 1H), 6.90-6.82 (m, 1H), 4.98-4.85 (m, 1.5H), 4.75 (d, J = 9.3 Hz, 0.5H), 4.64 (d, J = 9.0 Hz, 0.5H), 4.50-4.33 (m, 2.5H), 4.31-4.20 (m, 1H), 4.12-4.05 (m, 0.5H), 4.04-3.97 (m, 0.5H), 3.55 (d, J = 4.9 Hz, 3H), 2.90 (d, J = 9.5 Hz, 3H), 2.40-2.27 (m, 1H), 2.26-1.62 (m, 9H). |
| 30 | ((2- (((3S,6S,7aS,8aR, 9aR)-8,8-difluoro- 3-(3-(morpholine- 4-carbonyl)azetidine- 1-carbonyl)-5- oxodecahydro-1H- cyclopropa[d] pyrrolo[1,2-a]azocin- 6-yl)carbamoyl) benzo[b]thiophen-5- yl)difluoromethyl) phosphonic acid | 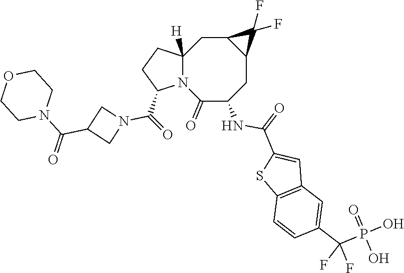 [SMILES: O=C(N[C@H]1C[C@H]2[C@@H](C[C@H]3CC[C@@H](C(=O)N4CC(C(=O)N5CCOCC5)C4)N3C1=O)C2(F)F)c1cc2cc(C(F)(F)P(=O)(O)O)ccc2s1] | 717.2 [M + H]⁺ | ¹H NMR (400 MHz, CD₃OD) δ 8.16 (s, 2H), 8.01 (d, J = 8.3 Hz, 1H), 7.69 (d, J = 8.6 Hz, 1H), 5.01 (t, J = 8.8 Hz, 1H), 4.73-4.64 (m, 1H), 4.55-4.49 (m, 1H), 4.49-4.39 (m, 1H), 4.38-4.31 (m, 1H), 4.27- 4.18 (m, 1H), 4.14-4.03 (m, 1H), 3.86-3.73 (m, 1H), 3.70-3.53 (m, 6H), 3.42-3.36 (m, 1H), 3.36-3.32 (m, 1H), 2.42-2.08 (m, 8H), 2.02- 1.94 (m, 1H), 1.84-1.71 (m, 1H). |
| 31 | ((2- (((3S,6S,7aS, 8aR,9aR)- 8,8-difluoro- 5-oxo-3-(3- (pyridin-3-yl) azetidine-1- carbonyl)decahydro- 1H-cyclopropa[d] pyrrolo[1,2-a]azocin- 6-yl)carbamoyl) benzo[b]thiophen-5- yl)difluoromethyl) phosphonic acid | 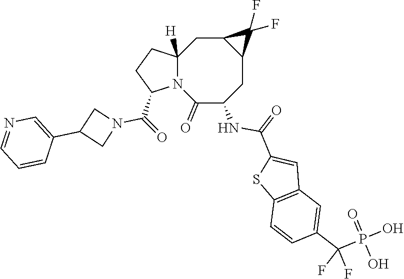 [SMILES: O=C(N[C@H]1C[C@H]2[C@@H](C[C@H]3CC[C@@H](C(=O)N4CC(c5cccnc5)C4)N3C1=O)C2(F)F)c1cc2cc(C(F)(F)P(=O)(O)O)ccc2s1] | 681.0 [M + H]⁺ | ¹H NMR (400 MHz, CD₃OD: D₂O) δ 8.65-8.55 (m, 1H), 8.53- 8.45 (m, 1H), 8.28-8.23 (m, 0.5H), 8.22-8.14 (m, 2H), 8.10-8.02 (m, 0.5H), 7.97-7.91 (m, 1H), 7.76- 7.71 (m, 1H), 7.63-7.56 (m, 1H), 5.06-4.98 (m, 1.5H), 4.75-4.70 (m, 0.5H), 4.68-4.62 (m, 0.5H), 4.61-4.49 (m, 1.5H), 4.46-4.29 (m, 2H), 4.09-3.98 (m, 2H), 2.42- 2.30 (m, 3H), 2.29-2.11 (m, 5H), 2.07-1.96 (m, 1H), 1.90-1.75 (m, 1H). |
| 32 | ((2- (((3S,6S,7aS,8aR, 9aR)-3-([1,1′- biphenyl]-4- ylcarbamoyl)-5- oxodecahydro-1H- cyclopropa[d] pyrrolo[1,2-a]azocin- 6-yl)carbamoyl) benzo[b]thiophen-5- yl)difluoromethyl) phosphonic acid | 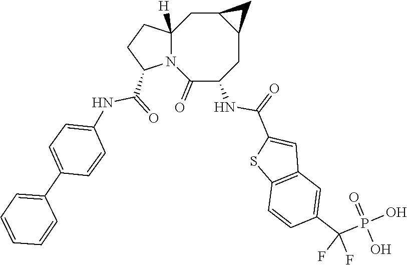 [SMILES: O=C(N[C@H]1C[C@@H]2C[C@@H]2C[C@H]2CC[C@@H](C(=O)Nc3ccc(-c4ccccc4)cc3)N2C1=O)c1cc2cc(C(F)(F)P(=O)(O)O)ccc2s1] | 680.3 [M + H]⁺ | ¹H NMR (400 MHz, CD₃OD) δ 8.20-8.12 (m, 2H), 8.02 (d, J = 8.3 Hz, 1H), 7.68 (d, J = 8.6 Hz, 1H), 7.63-7.52 (m, 6H), 7.41 (t, J = 7.6 Hz, 2H), 7.34-7.26 (m, 1H), 5.10 (t, J = 9.0 Hz, 1H), 4.81 (d, J = 7.3 Hz, 1H), 4.43-4.29 (m, 1H), 2.56- 2.43 (m, 1H), 2.36-2.06 (m, 5H), 1.99 (dt, J = 15.0, 8.9 Hz, 1H), 1.81-1.67 (m, 1H), 1.24-1.10 (m, 1H), 0.99-0.87 (m, 1H), 0.87-0.78 (m, 1H), 0.08 (q, J = 5.1 Hz, 1H). |
| 33 | Linker SFC Peak 1 used ((R)-fluoro(2- (((3S,6S,7aS,8aR, 9aR)-3-(3- (morpholine-4- carbonyl)azetidine- 1-carbonyl)-5-oxo- decahydro-1H- cyclopropa[d] pyrrolo[1,2-a]azocin- 6-yl)carbamoyl) benzo[b]thiophen-5- yl)methyl) phosphonic acid or ((S)-fluoro(2-(((3S, 6S,7aS,8aR,9aR)-3- (3-(morpholine-4- carbonyl)azetidine- 1-carbonyl)-5- oxodecahydro-1H- cyclopropa[d] pyrrolo[1,2-a]azocin- 6-yl)carbamoyl) benzo[b]thiophen-5- yl)methyl) phosphonic acid | 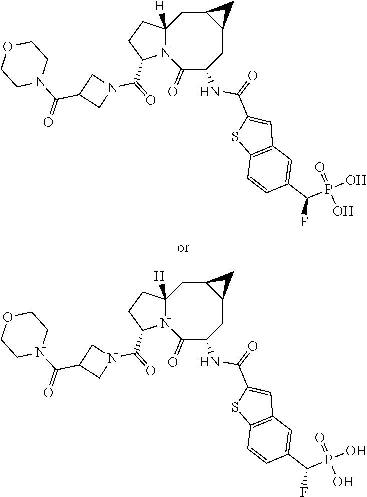 [SMILES: O=C(N[C@H]1C[C@@H]2C[C@@H]2C[C@H]2CC[C@@H](C(=O)N3CC(C(=O)N4CCOCC4)C3)N2C1=O)c1cc2cc([C@H](F)P(=O)(O)O)ccc2s1 ; O=C(N[C@H]1C[C@@H]2C[C@@H]2C[C@H]2CC[C@@H](C(=O)N3CC(C(=O)N4CCOCC4)C3)N2C1=O)c1cc2cc([C@@H](F)P(=O)(O)O)ccc2s1] | 663.1 [M + H]⁺ | ¹H NMR (400 MHz, CD₃OD) δ 8.14-8.07 (m, 1H), 8.04-7.98 (m, 1H), 7.97-7.90 (m, 1H), 7.63- 7.55 (m, 1H), 5.83 (dd, J = 45.2, 7.6 Hz, 1H), 5.08-4.98 (m, 1H), 4.72 (q, J = 8.8 Hz, 1H), 4.53-4.37 (m, 2H), 4.34-4.16 (m, 2H), 4.11- 4.02 (m, 1H), 3.84-3.71 (m, 1H), 3.62-3.44 (m, 6H), 3.40-3.32 (m, 2H), 2.50-2.38 (m, 1H), 2.35-2.07 (m, 4H), 2.03-1.90 (m, 2H), 1.79- 1.66 (m, 1H), 1.49-1.35 (m, 1H), 1.00-0.88 (m, 1H), 0.87-0.78 (m, 1H), 0.11-0.01 (m, 1H). |
| 34 | Linker SFC Peak 2 used ((S)- fluoro(2-(((3S,6S, 7aS,8aR,9aR)-3-(3- (morpholine-4- carbonyl)azetidine-1- carbonyl)-5- oxodecahydro-1H- cyclopropa[d] pyrrolo[1,2-a]azocin- 6-yl)carbamoyl) benzo[b]thiophen-5- yl)methyl) phosphonic acid Or ((R)-fluoro(2-(((3S, 6S,7aS,8aR,9aR)-3- (3-(morpholine-4- carbonyl)azetidine-1- carbonyl)-5-oxo- decahydro-1H- cyclopropa[d] pyrrolo[1,2-a] azocin-6-yl) carbamoyl)benzo[b] thiophen-5-yl) methyl) phosphonic acid | 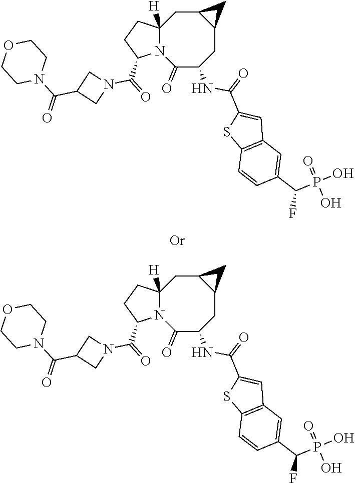 [SMILES: O=C(N[C@H]1C[C@@H]2C[C@@H]2C[C@H]2CC[C@@H](C(=O)N3CC(C(=O)N4CCOCC4)C3)N2C1=O)c1cc2cc([C@@H](F)P(=O)(O)O)ccc2s1 ; O=C(N[C@H]1C[C@@H]2C[C@@H]2C[C@H]2CC[C@@H](C(=O)N3CC(C(=O)N4CCOCC4)C3)N2C1=O)c1cc2cc([C@H](F)P(=O)(O)O)ccc2s1] | 663.1 [M + H]⁺ | ¹H NMR (400 MHz, CD₃OD) δ 8.14-8.07 (m, 1H), 8.05-7.98 (m, 1H), 7.97-7.89 (m, 1H), 7.64-7.55 (m, 1H), 5.81 (dd, J = 44.7, 8.3 Hz, 1H), 5.09-4.99 (m, 1H), 4.72 (q, J = 9.0 Hz, 1H), 4.54-4.37 (m, 2H), 4.34-4.16 (m, 2H), 4.11-4.02 (m, 1H), 3.85-3.71 (m, 1H), 3.69- 3.51 (m, 6H), 3.42-3.32 (m, 2H), 2.51-2.36 (m, 1H), 2.35-2.22 (m, 2H), 2.21-2.06 (m, 2H), 2.04-1.90 (m, 2H), 1.81-1.67 (m, 1H), 1.47- 1.34 (m, 1H), 1.00-0.88 (m, 1H), 0.88-0.79 (m, 1H), 0.11-0.01 (m, 1H). |
| 35 | (1-(2- (((3S,6S,7aS,8aR, 9aR)-3-(3- (morpholine-4- carbonyl)azetidine- 1-carbonyl)-5- oxodecahydro-1H- cyclopropa[d] pyrrolo[1,2-a]azocin- 6-yl)carbamoyl) benzo[b]thiophen- 5-yl)ethyl) phosphonic acid | 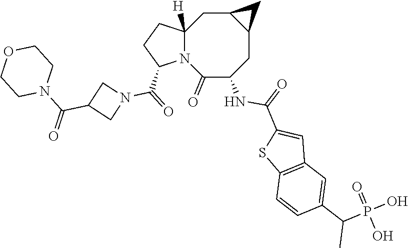 [SMILES: CC(c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1)P(=O)(O)O] | 659.1 [M + H]⁺ | ¹H NMR (400 MHz, CD₃OD) δ 8.04 (s, 1H), 7.93-7.87 (m, 1H), 7.84 (d, J = 8.3 Hz, 1H), 7.48 (d, J = 8.6 Hz, 1H), 5.03 (t, J = 9.2 Hz, 1H), 4.55-4.46 (m, 1H), 4.33- 4.19 (m, 1H), 3.78-3.42 (m, 10H), 3.40-3.34 (m, 2H), 3.30-3.21 (m, 2H), 2.51-2.40 (m, 1H), 2.34-2.25 (m, 1H), 2.25-1.91 (m, 5H), 1.77- 1.69 (m, 1H), 1.68-1.58 (m, 3H), 1.18-1.05 (m, 1H), 1.05-0.93 (m, 1H), 0.93-0.82 (m, 1H), 0.10 (q, J = 5.0 Hz, 1H). |
| 36 | (difluoro(2- (((3S,6S,7aS, 8aR,9aR)-5- oxo-3-(3- (pyridin-3- yl)azetidine-1- carbonyl)decahydro- 1H-cyclopropa[d] pyrrolo[1,2-a]azocin- 6-yl)carbamoyl) benzo[b]thiophen-5- yl)methyl) phosphonic acid | 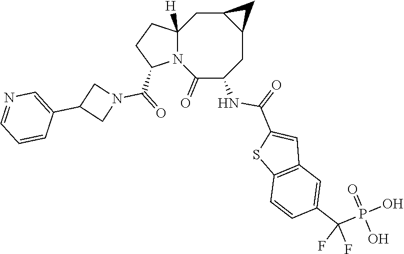 [SMILES: O=C(N[C@H]1C[C@@H]2C[C@@H]2C[C@H]2CC[C@@H](C(=O)N3CC(c4cccnc4)C3)N2C1=O)c1cc2cc(C(F)(F)P(=O)(O)O)ccc2s1] | 645.0 [M + H]⁺ | ¹H NMR (400 MHz, CD₃OD: NaOD) δ 8.59-8.53 (m, 1H), 8.50- 8.42 (m, 1H), 8.31-8.22 (m, 1H), 8.18-8.09 (m, 1.5H), 7.98-7.92 (m, 0.5H), 7.91-7.80 (m, 2H), 7.53-7.43 (m, 1H), 5.01 (t, J = 8.6 Hz, 0.5H), 4.76-4.68 (m, 0.5H), 4.66-4.52 (m, 2H), 4.50-4.39 (m, 1.5H), 4.34-4.27 (m, 0.5H), 4.14- 3.95 (m, 2H), 3.79 (dd, J = 11.1, 6.2 Hz, 1H), 2.58-2.48 (m, 1H), 2.42-2.31 (m, 1H), 2.25-2.04 (m, 5H), 1.62-1.49 (m, 1H), 1.49-1.37 (m, 1H), 1.15-1.02 (m, 1H), 0.81- 0.72 (m, 1H), 0.32-0.22 (m, 1H). |
| 37 | (difluoro(2- (((3S,6S,7aS,8aR, 9aR)-7a-methyl-5- oxo-3-(3-(pyridin- 3-yl)azetidine-1- carbonyl) decahydro-1H- cyclopropa[d] pyrrolo[1,2-a]azocin- 6-yl)carbamoyl) benzo[b]thiophen- 5-yl)methyl) phosphonic acid | 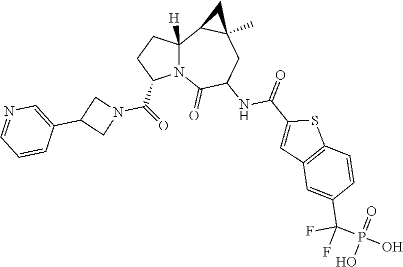 [SMILES: C[C@]12CC(NC(=O)c3cc4cc(C(F)(F)P(=O)(O)O)ccc4s3)C(=O)N3[C@H](CC[C@H]3C(=O)N3CC(c4cccnc4)C3)[C@H]1C2] | 645.1 [M + H]⁺ | ¹H NMR (400 MHz, CD₃OD) δ 8.57-8.48 (m, 1H), 8.47-8.38 (m, 1H), 8.29-8.23 (m, 1H), 8.11-7.92 (m, 2H), 7.90-7.81 (m, 2H), 7.52- 7.39 (m, 1H), 5.01-4.90 (m, 1H), 4.71-4.63 (m, 1H), 4.63-4.55 (m, 2H), 4.45-4.24 (m, 1H), 4.10-3.95 (m, 2H), 3.85-3.74 (m, 1H), 2.45- 2.27 (m, 1H), 2.25-2.06 (m, 4H), 2.06-1.95 (m, 1H), 1.32-1.17 (m, 4H), 0.77-0.70 (m, 1H), 0.67-0.60 (m, 1H). |
| 38 | (difluoro(2- (((3S,6S,7aR,8aS, 9aR)-7a-methyl-5- oxo-3-(3-(pyridin- 3-yl)azetidine-1- carbonyl)decahydro- 1H-cyclopropa[d] pyrrolo[1,2-a] azocin-6-yl) carbamoyl)benzo[b] thiophen-5-yl) methyl)phosphonic acid | 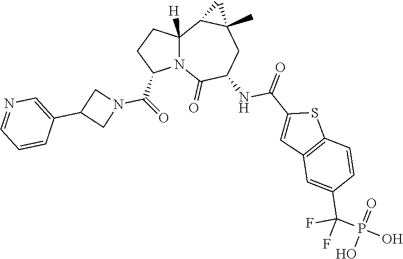 [SMILES: C[C@@]12C[C@H](NC(=O)c3cc4cc(C(F)(F)P(=O)(O)O)ccc4s3)C(=O)N3[C@H](CC[C@H]3C(=O)N3CC(c4cccnc4)C3)[C@@H]1C2] | 645.1 [M + H]⁺ | ¹H NMR (400 MHz, CD₃OD) δ 8.57-8.48 (m, 1H), 8.47-8.38 (m, 1H), 8.29-8.23 (m, 1H), 8.11- 7.92 (m, 2H), 7.90-7.81 (m, 2H), 7.52-7.39 (m, 1H), 5.01-4.90 (m, 1H), 4.71-4.63 (m, 1H), 4.63- 4.55 (m, 2H), 4.45-4.24 (m, 1H), 4.10-3.95 (m, 2H), 3.85-3.74 (m, 1H), 2.45-2.27 (m, 1H), 2.25-2.06 (m, 4H), 2.06-1.95 (m, 1H), 1.32- 1.17 (m, 4H), 0.77-0.70 (m, 1H), 0.67-0.60 (m, 1H). |
| 39 | (difluoro(2- (((3S,6S,7aS,8aR, 9aR)-5-oxo-3-(((R)- 1-(pyridin-2-yl) pyrrolidin-3-yl) carbamoyl) decahydro-1H- cyclopropa[d] pyrrolo[1,2-a] azocin-6-yl) carbamoyl)benzo[b] thiophen-5- yl)methyl) phosphonic acid | 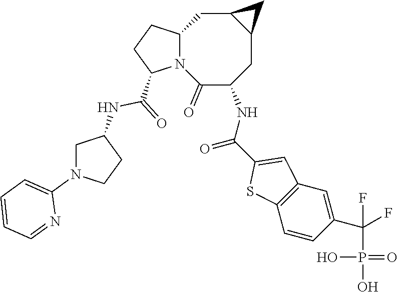 [SMILES: O=C(N[C@H]1C[C@@H]2C[C@@H]2C[C@H]2CC[C@@H](C(=O)N[C@@H]3CCN(c4ccccn4)C3)N2C1=O)c1cc2cc(C(F)(F)P(=O)(O)O)ccc2s1] | 674.4 [M + H]⁺ | ¹H NMR (400 MHz, CD₃OD) δ 8.16 (s, 1H), 8.06 (s, 1H), 7.92- 7.86 (m, 2H), 7.77-7.70 (m, 2H), 6.80-6.71 (m, 2H), 5.00-4.95 (m, 1H), 4.54 (d, J = 7.2 Hz, 1H), 4.51-4.45 (m, 1H), 4.31- 4.22 (m, 1H), 3.71-3.66 (m, 1H), 3.65-3.65 (m, 1H), 3.63-3.60 (m, 1H), 2.44-2.30 (m, 2H), 2.29-2.18 (m, 2H), 2.17-2.05 (m, 4H), 1.96-1.88 (m, 1H), 1.74- 1.63 (m, 1H), 1.32-1.26 (m, 1H), 1.13-1.01 (m, 1H), 0.82-0.78 (m, 1H), 0.68-0.62 (m, 1H), 0.03-0.00 (m, 1H). |
| 40 | ((2- (((3S,6S,7aS,8aR, 9aR)-3-(6-acetyl- 2,6-diazaspiro[3.3] heptane-2- carbonyl)-5- oxodecahydro-1H- cyclopropa[d] pyrrolo[1,2-a]azocin- 6-yl)carbamoyl) benzo[b]thiophen-5- yl)difluoromethyl) phosphonic acid | 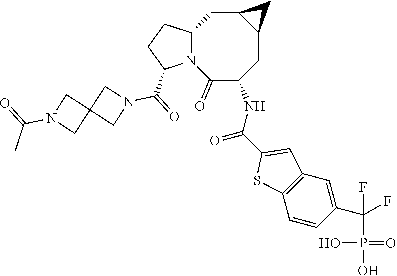 [SMILES: CC(=O)N1CC2(C1)CN(C(=O)[C@@H]1CC[C@@H]3C[C@H]4C[C@H]4C[C@H](NC(=O)c4cc5cc(C(F)(F)P(=O)(O)O)ccc5s4)C(=O)N31)C2] | 651.3 [M + H]⁺ | ¹H NMR (400 MHz, CD₃OD) δ 8.20-8.12 (m, 2H), 8.03 (d, J = 8.4 Hz, 1H), 7.67 (d, J = 8.4 Hz, 1H), 5.09-4.99 (m, 1H), 4.57- 4.49 (m, 1H), 4.36-4.22 (m, 1H), 4.06 (t, J = 8.8 Hz, 1H), 3.98-3.89 (m, 1H), 3.86-3.76 (m, 1H), 3.72- 3.60 (m, 2H), 3.58 (s, 2H), 3.50- 3.43 (m, 1H), 3.28-3.06 (m, 1H), 2.51-2.41 (m, 1H), 2.36-2.26 (m, 1H), 2.24-2.14 (m, 2H), 2.14-2.04 (m, 2H), 2.02-1.94 (m, 1H), 1.89 (d, J = 8.0 Hz, 3H), 1.79-1.65 (m, 1H), 1.30-1.20 (m, 1H), 1.03-0.92 (m, 1H), 0.92-0.82 (m, 1H), 0.15- 0.05 (m, 1H). |
| 41 | (difluoro(2- (((3S,6S,7aS,8aR, 9aR)-5-oxo-3-(6-(2- phenylacetyl)-2,6- diazaspiro[3.3] heptane-2- carbonyl)decahydro- 1H- cyclopropa[d] pyrrolo[1,2-a]azocin- 6-yl)carbamoyl) benzo[b]thiophen-5- yl)methyl) phosphonic acid | 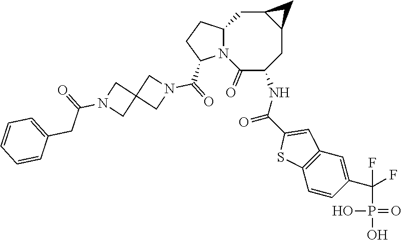 [SMILES: O=C(N[C@H]1C[C@@H]2C[C@@H]2C[C@H]2CC[C@@H](C(=O)N3CC4(CN(C(=O)Cc5ccccc5)C4)C3)N2C1=O)c1cc2cc(C(F)(F)P(=O)(O)O)ccc2s1] | 727.3 [M + H]⁺ | ¹H NMR (400 MHz, CD₃OD) δ 8.18 (d, J = 3.6 Hz, 1H), 8.14- 8.10 (m, 1H), 7.96-7.89 (m, 1H), 7.74 (d, J = 8.8 Hz, 1H), 7.33-7.17 (m, 5H), 5.05-4.98 (m, 1H), 4.71- 4.65 (m, 1H), 4.47-4.42 (m, 1H), 4.40-4.32 (m, 3H), 4.32-4.23 (m, 1H), 4.18-4.11 (m, 3H), 4.08- 4.02 (m, 1H), 3.48 (d, J = 3.6 Hz, 2H), 2.51-2.38 (m, 1H), 2.28 (m, 1H), 2.25-2.04 (m, 3H), 2.01-1.86 (m, 2H), 1.77-1.66 (m, 1H), 1.40 (m, 1H), 0.98-0.87 (m, 1H), 0.87- 0.79 (m, 1H), 0.08-0.02 (m, 1H). |
| 42 | (difluoro(2- (((3S,6S,7aS,8aR, 9aR)-3-(4-(oxetane- 3-carbonyl) piperazine-1- carbonyl)-5- oxodecahydro-1H- cyclopropa[d] pyrrolo[12-a]azocin- 6-yl)carbamoyl) benzo[b]thiophen-5- yl)methyl) phosphonic acid | 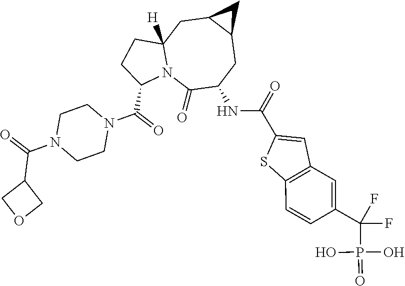 [SMILES: O=C(N[C@H]1C[C@@H]2C[C@@H]2C[C@H]2CC[C@@H](C(=O)N3CCN(C(=O)C4COC4)CC3)N2C1=O)c1cc2cc(C(F)(F)P(=O)(O)O)ccc2s1] | 681.1 [M + H]⁺ | ¹H NMR (400 MHz, CD₃OD) δ 8.18 (s, 1H), 8.12 (s, 1H), 7.92 (d, J = 8.6 Hz, 1H), 7.75 (d, J = 8.4 Hz, 1H), 5.04 (t, J = 8.8 Hz, 1H), 4.95 (dd, J = 8.0, 14.4 Hz, 1H), 4.83 (d, J = 7.6 Hz, 6H), 4.37- 4.14 (m, 2H), 3.92-3.37 (m, 6H), 2.53-2.41 (m, 1H), 2.32 (d, J = 12.4 Hz, 1H), 2.19 (s, 3H), 2.06- 1.87 (m, 2H), 1.85-1.66 (m, 1H), 1.43 (m, 1H), 1.09-0.94 (m, 1H), 0.92-0.81 (m, 1H), 0.07 (d, J = 4.8 Hz, 1H). |
| 43 | (difluoro(2- (((3S,6S,7aS,8aR, 9aR)-3-(4-(oxetan- 3-yl)piperazine-1- carbonyl)-5- oxodecahydro-1H- cyclopropa[d] pyrrolo[1,2-a] azocin-6-yl) carbamoyl)benzo[b] thiophen-5- yl)methyl) phosphonic acid | 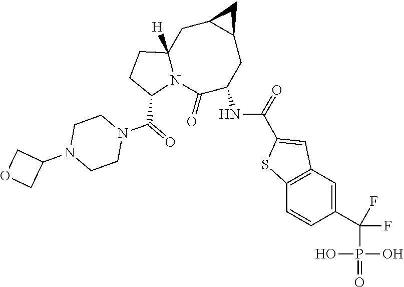 [SMILES: O=C(N[C@H]1C[C@@H]2C[C@@H]2C[C@H]2CC[C@@H](C(=O)N3CCN(C4COC4)CC3)N2C1=O)c1cc2cc(C(F)(F)P(=O)(O)O)ccc2s1] | 653.2 [M + H]⁺ | ¹H NMR (400 MHz, DMSO-d₆) δ 8.84 (d, J = 7.2 Hz, 1H), 8.31 (s, 1H), 8.14-8.01 (m, 2H), 7.60 (d, J = 8.4 Hz, 1H), 4.96-4.84 (m, 2H), 4.66-4.58 (m, 4H), 4.17 (d, J = 6.0 Hz, 1H), 4.01 (d, J = 5.2 Hz, 1H), 3.78 (s, 2H), 3.64-3.52 (m, 2H), 2.79 (s, 4H), 2.37 (m, 1H), 2.22-1.95 (m, 4H), 1.91- 1.75 (m, 2H), 1.66 (dd, J = 7.2, 12.0 Hz, 1H), 1.38 (d, J = 3.6 Hz, 1H), 0.91-0.79 (m, 1H), 0.74 (m, 1H), 0.02 (d, J = 5.2 Hz, 1H). |
| 44 | (difluoro(2- (((3S,6S,7aS,8aR, 9aR)-5-oxo-3-((1- (pyridin-2-yl) azetidin-3-yl) carbamoyl) decahydro-1H- cyclopropa[d] pyrrolo[1,2-a] azocin-6-yl) carbamoyl)benzo[b] thiophen-5-yl) methyl)phosphonic acid | 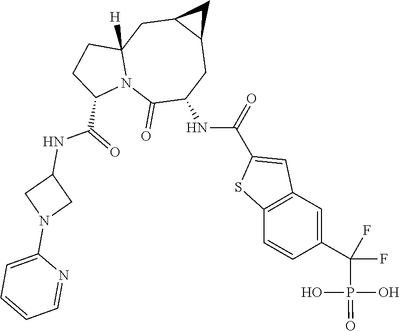 [SMILES: O=C(N[C@H]1C[C@@H]2C[C@@H]2C[C@H]2CC[C@@H](C(=O)NC3CN(c4ccccn4)C3)N2C1=O)c1cc2cc(C(F)(F)P(=O)(O)O)ccc2s1] | 660 [M + H]⁺ | ¹H NMR (400 MHz, CD₃OD) δ 8.08 (s, 1H), 8.02 (s, 1H), 7.90 (dd, J = 1.2, 4.8 Hz, 1H), 7.83 (d, J = 8.4 Hz, 1H), 7.64 (d, J = 8.8 Hz, 1H), 7.49-7.45 (m, 1H), 6.59 (dd, J = 5.6, 6.6 Hz, 1H), 6.35 (d, J = 8.4 Hz, 1H), 4.95 (t, J = 9.2 Hz, 1H), 4.60-4.45 (m, 2H), 4.21 (q, J = 8.0 Hz, 3H), 3.77-3.70 (m, 2H), 2.43-2.30 (m, 1H), 2.24- 1.95 (m, 5H), 1.92-1.84 (m, 1H), 1.67-1.58 (m, 1H), 1.14-1.00 (m, 1H), 0.86-0.81 (m, 1H), 0.78- 0.70 (m, 1H), −0.01 (q, J = 5.2 Hz, 1H). |
| 45 | (difluoro(2- (((3S,6S,7aR,8aS, 9aR)-5-oxo-3-(3- (pyridin-3-yl) azetidine-1- carbonyl)decahydro- 1H-cyclopropa[d] pyrrolo[1,2-a]azocin- 6-yl)carbamoyl) benzo[b]thiophen-5- yl)methyl) phosphonic acid | 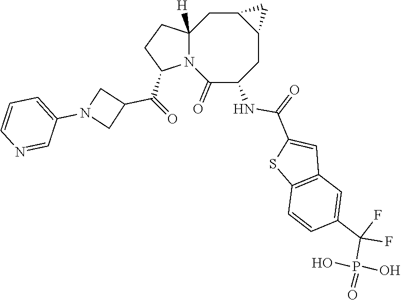 [SMILES: O=C(N[C@H]1C[C@H]2C[C@H]2C[C@H]2CC[C@@H](C(=O)C3CN(c4cccnc4)C3)N2C1=O)c1cc2cc(C(F)(F)P(=O)(O)O)ccc2s1] | 645.3 [M + H]⁺ | ¹H NMR (400 MHz, CD₃OD) δ 8.57-8.51 (m, 1H), 8.48-8.41 (m, 1H), 8.23-8.08 (m, 3H), 7.96-7.90 (m, 1H), 7.75 (d, J = 8.8 Hz, 1H), 7.54-7.47 (m, 1H), 4.95 (m, 1H), 4.72-4.56 (m, 1H), 4.54-4.22 (m, 4H), 4.06-3.93 (m, 2H), 2.42-2.34 (m, 1H), 2.31- 2.22 (m, 1H), 2.20-2.11 (m, 2H), 2.07-1.93 (m, 2H), 1.92-1.84 (m, 1H), 1.77-1.65 (m, 1H), 1.51- 1.42 (m, 1H), 1.33-1.23 (m, 1H), 0.90-0.82 (m, 1H), 0.21-0.15 (m, 1H). |
| 46 | ((7-(((3S,6S,7aS, 8aR,9aR)-3-(3-(1H- imidazol-1-yl) azetidine-1- carbonyl)-5- oxodecahydro-1H- cyclopropa[d]pyrrolo [1,2-a]azocin-6-yl) carbamoyl) naphthalen-2-yl) difluoromethyl) phosphonic acid | 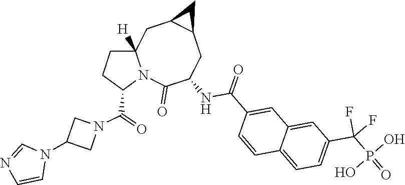 [SMILES: O=C(N[C@H]1C[C@@H]2C[C@@H]2C[C@H]2CC[C@@H](C(=O)N3CC(n4ccnc4)C3)N2C1=O)c1ccc2ccc(C(F)(F)P(=O)(O)O)cc2c1] | 628 [M + H]⁺ | ¹H NMR (400 MHz, CD₃OD) δ 9.10 (d, J = 12.4 Hz, 1H), 8.55- 8.43 (m, 1H), 8.26 (s, 1H), 8.08 (s, 1H), 8.00-7.92 (m, 3H), 7.85 (d, J = 8.8 Hz, 1H), 7.64-7.44 (m, 1H), 5.46-5.29 (m, 1H), 5.23-5.00 (m, 2H), 4.67-4.41 (m, 3H), 4.35 (s, 1H), 4.28-4.16 (m, 1H), 2.56- 2.44 (m, 1H), 2.35-2.12 (m, 4H), 2.10-1.94 (m, 2H), 1.76 (m, 1H), 1.46-1.28 (m, 1H), 1.05-0.93 (m, 1H), 0.89-0.79 (m, 1H), 0.08 (m, 1H). |
| 47 | (difluoro(2- (((3S,6S,7aR,8aS, 9aR)-5-oxo-3-(((R)- 1-(pyridin-3-yl) pyrrolidin-3-yl) carbamoyl) decahydro-1H- cyclopropa[d] pyrrolo[1,2-a]azocin- 6-yl)carbamoyl) benzo[b]thiophen-5- yl)methyl) phosphonic acid | 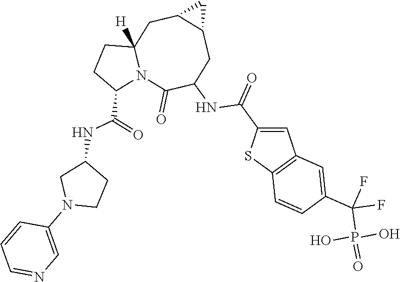 [SMILES: O=C(NC1C[C@H]2C[C@H]2C[C@H]2CC[C@@H](C(=O)N[C@@H]3CCN(c4cccnc4)C3)N2C1=O)c1cc2cc(C(F)(F)P(=O)(O)O)ccc2s1] | 674 [M + H]⁺ | ¹H NMR (400 MHz, CD₃OD) δ 8.03 (s, 1H), 7.92 (s, 1H), 7.80 (d, J = 8.4 Hz, 1H), 7.72 (s, 1H), 7.68-7.62 (m, 2H), 7.58-7.50 (m, 1H), 7.43 (d, J = 8.0 Hz, 1H), 4.92-4.88 (m, 2H), 4.46 (d, J = 2.0 Hz, 1H), 4.35-4.24 (m, 2H), 3.59- 3.51 (m, 2H), 3.46-3.37 (m, 1H), 2.47-2.37 (m, 1H), 2.36-2.22 (m, 2H), 2.21-2.05 (m, 3H), 2.04-1.84 (m, 3H), 1.76-1.63 (m, 1H), 1.50- 1.40 (m, 1H), 1.34-1.25 (m, 1H), 0.94-0.83 (m, 1H), 0.23-0.17 (m, 1H) |
| 48 | (difluoro(2- (((3S,6S,7aS,8aR, 9aR)-3-(((R)-1- (oxetan-3-yl) pyrrolidin-3-yl) carbamoyl)-5- oxodecahydro-1H- cyclopropa[d] pyrrolo[1,2-a] azocin-6-yl) carbamoyl)benzo [b]thiophen-5- yl)methyl) phosphonic acid | 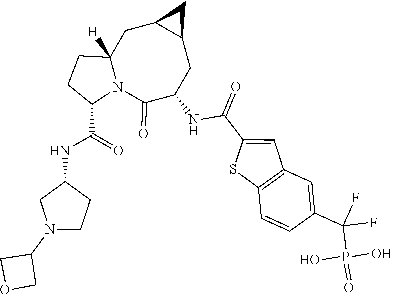 [SMILES: O=C(N[C@H]1C[C@@H]2C[C@@H]2C[C@H]2CC[C@@H](C(=O)N[C@@H]3CCN(C4COC4)C3)N2C1=O)c1cc2cc(C(F)(F)P(=O)(O)O)ccc2s1] | 653.1 [M + H]⁺ | ¹H NMR (400 MHz, DMSO-d₆) δ 8.81 (d, J = 7.2 Hz, 1H), 8.53- 8.26 (m, 2H), 8.15-8.01 (m, 2H), 7.60 (d, J = 8.8 Hz, 1H), 4.94 (d, J = 7.2 Hz, 1H), 4.73-4.62 (m, 2H), 4.61-4.55 (m, 1H), 4.50 (s, 1H), 4.40-4.25 (m, 3H), 4.16 (d, J = 6.0 Hz, 1H), 3.68 (d, J = 4.0 Hz, 1H), 3.35-3.20 (m, 2H), 2.73-2.63 (m, 1H), 2.40-2.32 (m, 1H), 2.25-2.07 (m, 3H), 2.04-1.93 (m, 2H), 1.93-1.85 (m, 2H), 1.83 (d, J = 9.2 Hz, 1H), 1.71-1.59 (m, 1H), 1.23 (s, 1H), 0.90-0.68 (m, 2H), 0.03 (m, 1H). |
| 49 | ((2- (((3S,6S,7aS,8aR, 9aR)-3-(3-(3- cyanophenyl) azetidine-1- carbonyl)-5- oxodecahydro-1H- cyclopropa[d] pyrrolo[1,2-a]azocin- 6-yl)carbamoyl) benzo[b]thiophen-5- yl)difluoromethyl) phosphonic acid | 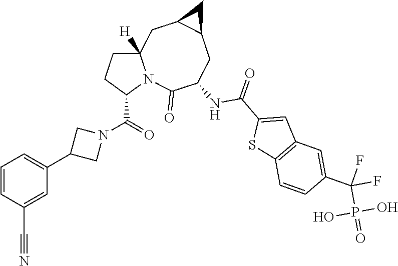 [SMILES: N#Cc1cccc(C2CN(C(=O)[C@@H]3CC[C@@H]4C[C@H]5C[C@H]5C[C@H](NC(=O)c5cc6cc(C(F)(F)P(=O)(O)O)ccc6s5)C(=O)N43)C2)c1] | 669.2 [M + H]⁺ | ¹H NMR (400 MHz, CD₃OD) δ 8.25-8.05 (m, 2H), 7.95-7.91 (m, 1H), 7.84 (s, 1H), 7.78-7.71 (m, 2H), 7.67-7.59 (m, 1H), 7.57-7.51 (m, 1H), 5.13-4.96 (m, 1H), 4.73-4.66 (m, 1H), 4.62- 4.52 (m, 1H), 4.41-4.19 (m, 2H), 4.09-3.89 (m, 2H), 2.55-2.42 (m, 1H), 2.37-2.09 (m, 4H), 2.07-1.91 (m, 2H), 1.84-1.68 (m, 1H), 1.52- 1.25 (m, 2H), 0.79 (d, J = 1.6 Hz, 2H), 0.14-0.01 (m, 1H). |
| 50 | (difluoro(2- (((3S,6S,7aS,8aR, 9aR)-5-oxo-3-(3- (pyrazin-2-yl) azetidine-1- carbonyl) decahydro-1H- cyclopropa[d] pyrrolo[1,2-a]azocin- 6-yl)carbamoyl) benzo[b]thiophen-5- yl)methyl) phosphonic acid | 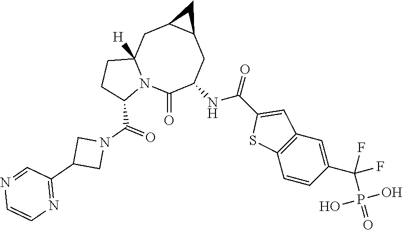 [SMILES: O=C(N[C@H]1C[C@@H]2C[C@@H]2C[C@H]2CC[C@@H](C(=O)N3CC(c4cnccn4)C3)N2C1=O)c1cc2cc(C(F)(F)P(=O)(O)O)ccc2s1] | 646.4 [M + H]⁺ | ¹H NMR (400 MHz, CD₃OD) δ 8.63-8.46 (m, 2H), 8.42 (dd, J = 2.4, 2.4 Hz, 1H), 8.14 (d, J = 9.6 Hz, 1H), 8.05 (d, J = 13.2 Hz, 1H), 7.83 (t, J = 8.4 Hz, 1H), 7.73- 7.67 (m, 1H), 4.98 (t, J = 8.8 Hz, 1H), 4.91-4.85 (m, 1H), 4.52-4.47 (m, 1H), 4.41-4.34 (m, 1H), 4.30-4.17 (m, 2H), 4.15-4.00 (m, 2H), 2.46-2.34 (m, 1H), 2.29- 2.18 (m, 2H), 2.16-2.04 (m, 2H), 1.98-1.86 (m, 2H), 1.73-1.62 (m, 1H), 1.41-1.32 (m, 1H), 0.97-0.86 (m, 1H), 0.79 (m, 1H), 0.06-0.06 (m, 1H). |
| 51 | (difluoro(2- (((3S,6S,7aS,8aR, 9aR)-5-oxo-3-(((R)- 1-(pyridazin-3- yl)pyrrolidin-3- yl)carbamoyl) decahydro-1H- cyclopropa[d] pyrrolo[1,2-a] azocin-6- yl)carbamoyl)benzo [b]thiophen-5-yl) methyl) phosphonic acid | 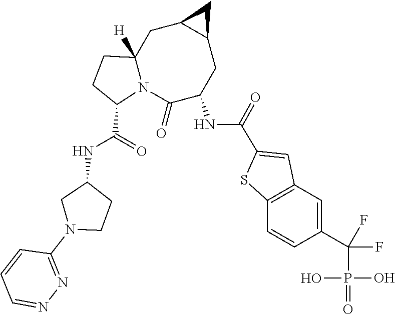 [SMILES: O=C(N[C@H]1C[C@@H]2C[C@@H]2C[C@H]2CC[C@@H](C(=O)N[C@@H]3CCN(c4cccnn4)C3)N2C1=O)c1cc2cc(C(F)(F)P(=O)(O)O)ccc2s1] | 675 [M + H]⁺ | ¹H NMR (400 MHz, CD₃OD) δ 8.49-8.36 (m, 1H), 8.17 (s, 1H), 8.10 (s, 1H), 7.91 (d, J = 8.4 Hz, 1H), 7.73 (d, J = 8.4 Hz, 1H), 7.38 (dd, J = 4.4, 9.2 Hz, 1H), 6.93 (dd, J = 1.2, 9.2 Hz, 1H), 4.62 (d, J = 7.2 Hz, 1H), 4.53-4.42 (m, 1H), 4.30-4.20 (m, 1H), 3.63-3.58 (m, 2H), 3.44 (dd, J = 2.4, 11.2 Hz, 1H), 2.39-2.28 (m, 2H), 2.26-2.16 (m, 3H), 2.13- 2.05 (m, 2H), 2.04-1.95 (m, 2H), 1.94-1.86 (m, 1H), 1.65 (m, 1H), 1.45-1.36 (m, 1H), 0.99-0.87 (m, 1H), 0.70 (m, 1H), 0.57 (m, 1H), −0.02 (m, 1H). |
| 52 | (difluoro(2- (((3S,6S,7aS,8aR, 9aR)-5-oxo-3-(((R)- 1-phenylpyrrolidin- 3-yl)carbamoyl) decahydro-1H- cyclopropa[d] pyrrolo[1,2-a]azocin- 6-yl)carbamoyl) benzo[b]thiophen- 5-yl)methyl) phosphonic acid | 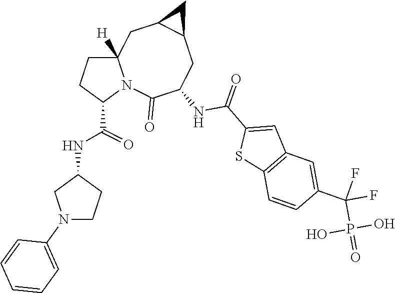 [SMILES: O=C(N[C@H]1C[C@@H]2C[C@@H]2C[C@H]2CC[C@@H](C(=O)N[C@@H]3CCN(c4ccccc4)C3)N2C1=O)c1cc2cc(C(F)(F)P(=O)(O)O)ccc2s1] | 673.1 [M + H]⁺ | ¹H NMR (400 MHz, CD₃OD) δ 8.29 (s, 1H), 8.21 (s, 1H), 8.02 (d, J = 8.4 Hz, 1H), 7.84 (d, J = 8.4 Hz, 1H), 7.26 (dd, J = 7.6, 8.4 Hz, 2H), 6.73 (t, J = 7.2 Hz, 1H), 6.62 (d, J = 8.0 Hz, 2H), 5.12- 5.06 (m, 1H), 4.75 (d, J = 7.2 Hz, 1H), 4.55 (m, 1H), 4.41-4.29 (m, 1H), 3.52 (dd, J = 5.6, 9.6 Hz, 2H), 3.43 (s, 1H), 3.24 (dd, J = 1.6, 10.0 Hz, 1H), 2.51-2.25 (m, 5H), 2.20-2.06 (m, 3H), 2.02- 1.91 (m, 1H), 1.83-1.65 (m, 1H), 0.99-0.76 (m, 2H), 0.50 (m, 1H), −0.01 (m, 1H). |
| 53 | (difluoro(2- (((3S,6S,7aS,8aR, 9aR)-5-oxo-3-((1- ((R)-tetrahydro- furan-3-yl)azetidin- 3-yl)carbamoyl) decahydro-1H- cyclopropa[d] pyrrolo[1,2-a]azocin- 6-yl)carbamoyl) benzo[b]thiophen-5- yl)methyl) phosphonic acid | 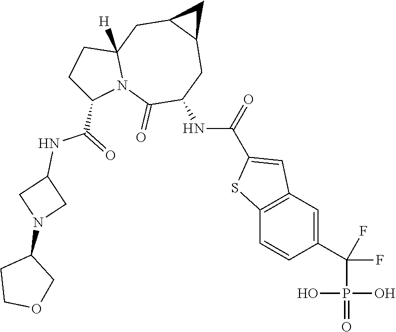 [SMILES: O=C(N[C@H]1C[C@@H]2C[C@@H]2C[C@H]2CC[C@@H](C(=O)NC3CN([C@@H]4CCOC4)C3)N2C1=O)c1cc2cc(C(F)(F)P(=O)(O)O)ccc2s1] | 653.1 [M + H]⁺ | ¹H NMR (400 MHz, CD₃OD) δ 8.11-8.02 (m, 2H), 7.86 (d, J = 9.2 Hz, 1H), 7.66-7.60 (m, 1H), 4.97- 4.89 (m, 1H), 4.43-4.35 (m, 1H), 4.45-4.34 (m, 1H), 4.29-4.17 (m, 3H), 4.05-4.02 (m, 2H), 3.96-3.83 (m, 2H), 3.64-3.51 (m, 2H), 2.37-2.31 (m, 1H), 2.23-1.78 (m, 9H), 1.69- 1.57 (m, 1H), 1.22-1.13 (m, 1H), 0.87-0.71 (m, 2H), 0.02-0.04 (m, 1H). |
| 54 | ((2- (((3S,6S,7aR,8aS, 9aR)-3-(3-(1H- imidazol-4-yl) azetidine-1- carbonyl)-5- oxodecahydro-1H- cyclopropa[d] pyrrolo[1,2-a] azocin-6-yl) carbamoyl)benzo[b] thiophen-5- yl)difluoromethyl) phosphonic acid | 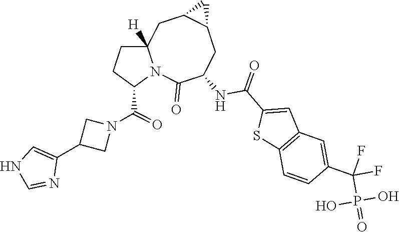 [SMILES: O=C(N[C@H]1C[C@H]2C[C@H]2C[C@H]2CC[C@@H](C(=O)N3CC(c4c[nH]cn4)C3)N2C1=O)c1cc2cc(C(F)(F)P(=O)(O)O)ccc2s1] | 634.2 [M + H]⁺ | ¹H NMR (400 MHz, CD₃OD) δ 8.22 (s, 1H), 8.12 (d, J = 4.0 Hz, 1H), 7.96-7.90 (m, 1H), 7.77 (d, J = 8.8 Hz, 1H), 7.67-7.58 (m, 1H), 7.12-6.98 (m, 1H), 4.94 (d, J = 4.4 Hz, 1H), 4.80 (s, 1H), 4.62-4.50 (m, 1H), 4.46-4.40 (m, 1H), 4.34-4.27 (m, 2H), 4.04-3.97 (m, 1H), 3.96-3.88 (m, 1H), 2.40- 2.31 (m, 1H), 2.25 (m, 1H), 2.20- 2.10 (m, 2H), 2.05-1.93 (m, 2H), 1.91-1.82 (m, 1H), 1.78-1.66 (m, 1H), 1.52-1.38 (m, 2H), 0.91-0.79 (m, 1H), 0.18 (d, J = 3.2 Hz, 1H). |
| 55 | (difluoro(2- (((3S,6S,7aR,8aS, 9aR)-3-(3-(2- methylpyridin-3- yl)azetidine-1- carbonyl)-5- oxodecahydro-1H- cyclopropa[d] pyrrolo[1,2-a]azocin- 6-yl)carbamoyl) benzo[b]thiophen- 5-yl)methyl) phosphonic acid | 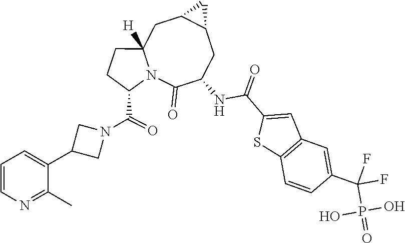 [SMILES: Cc1ncccc1C1CN(C(=O)[C@@H]2CC[C@@H]3C[C@@H]4C[C@@H]4C[C@H](NC(=O)c4cc5cc(C(F)(F)P(=O)(O)O)ccc5s4)C(=O)N32)C1] | 659.2 [M + H]⁺ | ¹H NMR (400 MHz, CD₃OD) δ 8.33-8.26 (m, 1H), 8.20 (m, 1H), 8.12 (m, 1H), 7.99-7.87 (m, 2H), 7.75 (d, J = 8.4 Hz, 1H), 7.33 (m, 1H), 4.98-4.95 (m, 1H), 4.71-4.60 (m, 1H), 4.54-4.26 (m, 4H), 4.24- 4.17 (m, 1H), 4.09-4.01 (m, 1H), 2.47 (d, J = 5.2 Hz, 3H), 2.40 (m, 1H), 2.27 (m, 1H), 2.22-2.11 (m, 2H), 2.07-1.95 (m, 2H), 1.88 (m, 1H), 1.75-1.64 (m, 1H), 1.51-1.39 (m, 1H), 1.33-1.25 (m, 1H), 0.89- 0.82 (m, 1H), 0.22-0.16 (m, 1H). |
| 56 | (difluoro(2- (((3S,6S,7aR,8aS, 9aR)-3-(3-(4- methylpyridin-3- yl)azetidine-1- carbonyl)-5- oxodecahydro-1H- cyclopropa[d] pyrrolo[1,2-a]azocin- 6-yl)carbamoyl) benzo[b]thiophen-5- yl)methyl) phosphonic acid | 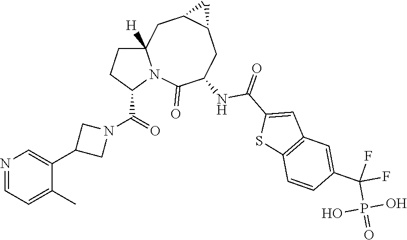 [SMILES: Cc1ccncc1C1CN(C(=O)[C@@H]2CC[C@@H]3C[C@@H]4C[C@@H]4C[C@H](NC(=O)c4cc5cc(C(F)(F)P(=O)(O)O)ccc5s4)C(=O)N32)C1] | 659.5 [M + H]⁺ | ¹H NMR (400 MHz, CD₃OD) δ 8.61-8.51 (m, 1H), 8.34-8.24 (m, 1H), 8.20-8.04 (m, 2H), 7.96-7.85 (m, 1H), 7.77-7.68 (m, 1H), 7.33- 7.23 (m, 1H), 4.95 (d, J = 6.4 Hz, 1H), 4.74-4.65 (m, 1H), 4.55- 4.27 (m, 4H), 4.26-4.06 (m, 2H), 2.44-2.35 (m, 1H), 2.30 (s, 3H), 2.26 (m, 1H), 2.20-2.11 (m, 2H), 2.05-1.94 (m, 2H), 1.92-1.80 (m, 1H), 1.77-1.63 (m, 1H), 1.45 (m, 1H), 1.34-1.22 (m, 1H), 0.91- 0.78 (m, 1H), 0.22-0.14 (m, 1H). |
| 57 | (difluoro(2- (((3S,6S,7aS,8aR, 9aR)-5-oxo-3-((1- ((S)-tetrahydrofuran- 3-yl)azetidin-3- yl)carbamoyl) decahydro-1H- cyclopropa[d] pyrrolo[1,2-a]azocin- 6-yl)carbamoyl) benzo[b]thiophen-5- yl)methyl) phosphonic acid | 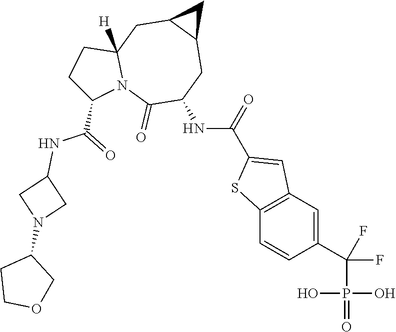 [SMILES: O=C(N[C@H]1C[C@@H]2C[C@@H]2C[C@H]2CC[C@@H](C(=O)NC3CN([C@H]4CCOC4)C3)N2C1=O)c1cc2cc(C(F)(F)P(=O)(O)O)ccc2s1] | 653.3 [M + H]⁺ | ¹H NMR (400 MHz, DMSO-d₆) δ 8.93 (d, J = 6.8 Hz, 1H), 8.28 (d, J = 6.8 Hz, 1H), 8.23 (s, 1H), 8.01 (s, 1H), 7.91 (d, J = 8.4 Hz, 1H), 7.60 (d, J = 8.4 Hz, 1H), 4.95-4.85 (m, 1H), 4.41 (d, J = 6.4 Hz, 1H), 4.16 (m, 2H), 3.64-3.61 (m, 2H), 3.51 (d, J = 4.0 Hz, 2H), 3.44 (s, 1H), 3.41-3.38 (m, 1H), 2.96 (s, 1H), 2.85 (s, 1H), 2.74 (t, J = 6.4 Hz, 1H), 2.15-2.07 (m, 2H), 1.95- 1.90 (m, 2H), 1.90-1.85 (m, 1H), 1.84-1.71 (m, 2H), 1.68-1.52 (m, 2H), 1.22-1.14 (m, 1H), 1.12- 1.03 (m, 1H), 0.87-0.70 (m, 2H), 0.03 (q, J = 4.8 Hz, 1H). |
| 58 | (difluoro(2- (((3S,6S,7aR,8aS, 9aR)-3-(3-(3- methylpyridin-2-yl) azetidine-1- carbonyl)-5- oxodecahydro-1H- cyclopropa[d] pyrrolo[1,2-a]azocin- 6-yl)carbamoyl) benzo[b]thiophen-5- yl)methyl) phosphonic acid | 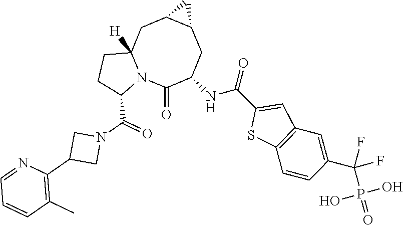 [SMILES: Cc1cccnc1C1CN(C(=O)[C@@H]2CC[C@@H]3C[C@@H]4C[C@@H]4C[C@H](NC(=O)c4cc5cc(C(F)(F)P(=O)(O)O)ccc5s4)C(=O)N32)C1] | 659.3 [M + H]⁺ | ¹H NMR (400 MHz, CD₃OD) δ 8.47-8.30 (m, 1H), 8.22-8.01 (m, 2H), 7.95-7.86 (m, 1H), 7.77-7.56 (m, 2H), 7.33-7.25 (m, 1H), 4.97- 4.91 (m, 2H), 4.85-4.76 (m, 1H), 4.71-4.50 (m, 1H), 4.49-4.37 (m, 2H), 4.36-4.24 (m, 2H), 2.41- 2.32 (m, 1H), 2.29 (d, J = 2.0 Hz, 3H), 2.24-2.11 (m, 2H), 2.06- 1.81 (m, 3H), 1.78-1.61 (m, 1H), 1.52-1.40 (m, 1H), 1.24 (s, 2H), 0.90-0.81 (m, 1H), 0.91 (m, 1H). |
| 59 | ((7- (((3S,6S,7aR,8aS, 9aR)-3-(3-(1H- imidazol-1-yl) azetidine-1- carbonyl)-5- oxodecahydro-1H- cyclopropa[d] pyrrolo[1,2-a]azocin- 6-yl)carbamoyl) naphthalen-2- yl)difluoromethyl) phosphonic acid | 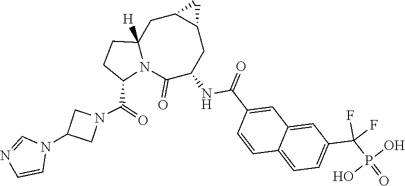 [SMILES: O=C(N[C@H]1C[C@H]2C[C@H]2C[C@H]2CC[C@@H](C(=O)N3CC(n4ccnc4)C3)N2C1=O)c1ccc2ccc(C(F)(F)P(=O)(O)O)cc2c1] | 628.3 [M + H]⁺ | ¹H NMR (400 MHz, CD₃OD) δ 8.72-8.57 (m, 1H), 8.53-8.45 (m, 1H), 8.30-8.27 (m, 1H), 8.14-7.99 (m, 1H), 7.99-7.90 (m, 3H), 7.89- 7.84 (m, 1H), 7.32-7.20 (m, 1H), 5.36-5.25 (m, 1H), 5.07-4.90 (m, 2H), 4.79 (m, 1H), 4.50-4.42 (m, 1H), 4.41-4.31 (m, 2H), 4.25-4.11 (m, 1H), 2.38 (m, 1H), 2.27-2.12 (m, 3H), 2.04-1.85 (m, 3H), 1.74- 1.62 (m, 1H), 1.50 (m, 1H), 1.37- 1.24 (m, 1H), 0.87 (m, 1H), 0.18 (m, 1H). |
| 60 | (difluoro(2- (((3S,6S,7aR,8aS, 9aR)-3-(3-(6- methylpyridin-2- yl)azetidine-1- carbonyl)-5- oxodecahydro-1H- cyclopropa[d] pyrrolo[1,2-a]azocin- 6-yl)carbamoyl) benzo[b]thiophen-5- yl)methyl) phosphonic acid | 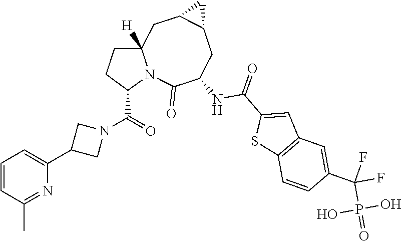 [SMILES: Cc1cccc(C2CN(C(=O)[C@@H]3CC[C@@H]4C[C@@H]5C[C@@H]5C[C@H](NC(=O)c5cc6cc(C(F)(F)P(=O)(O)O)ccc6s5)C(=O)N43)C2)n1] | 659.1 [M + H]⁺ | ¹H NMR (400 MHz, CD₃OD) δ 8.22-7.98 (m, 2H), 7.97-7.82 (m, 1H), 7.75 (m, 1H), 7.70-7.62 (m, 1H), 7.45-7.08 (m, 2H), 4.95 (m, 1H), 4.66 m, 1H), 4.50-4.39 (m, 2H), 4.32 (m, 1H), 4.23-4.08 (m, 1H), 4.08-3.98 (m, 1H), 2.57-2.41 (m, 3H), 2.41-2.23 (m, 2H), 2.22- 2.10 (m, 2H), 2.08-1.81 (m, 4H), 1.80-1.61 (m, 1H), 1.51-1.36 (m, 1H), 1.35-1.21 (m, 1H), 0.85 (m, 1H), 0.26-0.13 (m, 1H). |
| 61 | (difluoro(2- (((3S,6S,7aS,8aR, 9aR)-5-oxo-3-(3- (pyrimidin-5-yl) azetidine-1- carbonyl)decahydro- 1H-cyclopropa[d] pyrrolo[1,2-a]azocin- 6-yl)carbamoyl) benzo[b]thiophen- 5-yl)methyl) phosphonic acid | 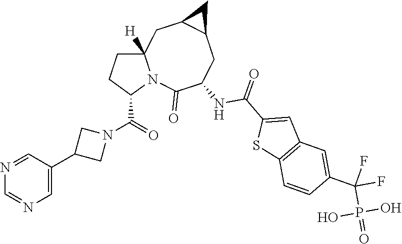 [SMILES: O=C(N[C@H]1C[C@@H]2C[C@@H]2C[C@H]2CC[C@@H](C(=O)N3CC(c4cncnc4)C3)N2C1=O)c1cc2cc(C(F)(F)P(=O)(O)O)ccc2s1] | 646 [M + H]⁺ | ¹H NMR (400 MHz, CD₃OD) δ 9.09-9.05 (m, 1H), 8.94 (s, 1H), 8.86 (s, 1H), 8.19-8.12 (m, 2H), 8.03-7.97 (m, 1H), 7.67 (m, 1H), 5.08-5.00 (m, 1H), 4.79-4.70 (m, 1H), 4.58-4.43 (m, 2H), 4.42-4.35 (m, 1H), 4.34-4.26 (m, 1H), 4.09- 3.91 (m, 2H), 2.54-2.43 (m, 1H), 2.36-2.09 (m, 4H), 2.06-1.92 (m, 2H), 1.80-1.68 (m, 1H), 1.49-1.37 (m, 1H), 1.02-0.91 (m, 1H), 0.90- 0.81 (m, 1H), 0.11-0.03 (m, 1H). |
| 62 | ((2- (((3S,6S,7aS,8aR, 9aR)-3-(3-(4- cyanophenyl)-3- fluoroazetidine-1- carbonyl)-5- oxodecahydro-1H- cyclopropa[d] pyrrolo[1,2-a]azocin- 6-yl)carbamoyl) benzo[b]thiophen-5- yl)difluoromethyl) phosphonic acid | 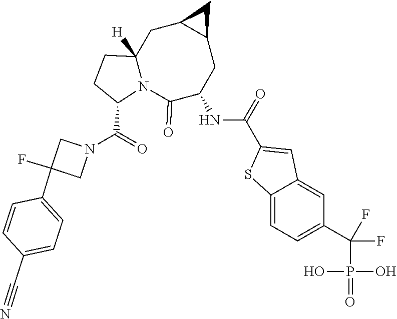 [SMILES: N#Cc1ccc(C2(F)CN(C(=O)[C@@H]3CC[C@@H]4C[C@H]5C[C@H]5C[C@H](NC(=O)c5cc6cc(C(F)(F)P(=O)(O)O)ccc6s5)C(=O)N43)C2)cc1] | 687.1 [M + H]⁺ | ¹H NMR (400 MHz, CD₃OD) δ 8.22-8.11 (m, 2H), 7.94 (d, J = 8.4 Hz, 1H), 7.86-7.81 (m, 1H), 7.79 (d, J = 3.2 Hz, 2H), 7.77-7.71 (m, 2H), 5.17-5.01 (m, 2H), 4.80- 4.64 (m, 1H), 4.57 (dd, J = 2.4, 8.4 Hz, 1H), 4.53-4.38 (m, 1H), 4.37-4.27 (m, 2H), 2.48 (m, 1H), 2.38-2.28 (m, 2H), 2.27-2.12 (m, 2H), 2.08-1.91 (m, 2H), 1.82-1.69 (m, 1H), 1.46-1.27 (m, 1H), 1.02- 0.91 (m, 1H), 0.90-0.79 (m, 1H), 0.08 (m, 1H). |
| 63 | (difluoro(2- (((3S,6S,7aR,8aS, 9aR)-5-oxo-3-((1- (4,4,4-trifluoro- butanoyl)azetidin-3- yl)carbamoyl) decahydro-1H- cyclopropa[d] pyrrolo[1,2-a]azocin- 6-yl)carbamoyl) benzo[b]thiophen-5- yl)methyl) phosphonic acid | 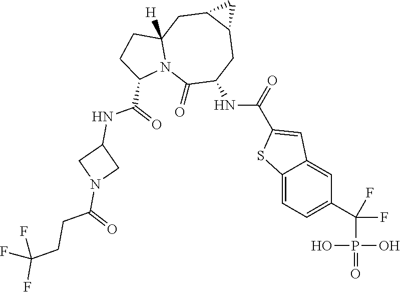 [SMILES: O=C(N[C@H]1C[C@H]2C[C@H]2C[C@H]2CC[C@@H](C(=O)NC3CN(C(=O)CCC(F)(F)F)C3)N2C1=O)c1cc2cc(C(F)(F)P(=O)(O)O)ccc2s1] | 707.2 [M + H]⁺ | ¹H NMR (400 MHz, CD₃OD) δ 8.16-8.10 (m, 2H), 8.00 (d, J = 8.6 Hz, 1H), 7.68 (m, 1H), 4.95 (m, 1H), 4.56-4.45 (m, 2H), 4.38-4.23 (m, 3H), 4.12-4.04 (m, 1H), 3.95-3.85 (m, 1H), 2.50- 2.32 (m, 5H), 2.27-2.11 (m, 3H), 2.01-1.84 (m, 3H), 1.79-1.68 (m, 1H), 1.52-1.41 (m, 1H), 1.34-1.26 (m, 1H), 0.89-0.82 (m, 1H), 0.22-0.16 (m, 1H). |
| 64 | (difluoro(2- (((3S,6S,7aS,8aR, 9aR)-3-(3-fluoro-3- phenylazetidine-1- carbonyl)-5- oxodecahydro-1H- cyclopropa[d] pyrrolo[1,2-a]azocin- 6-yl)carbamoyl) benzo[b]thiophen- 5-yl)methyl) phosphonic acid | 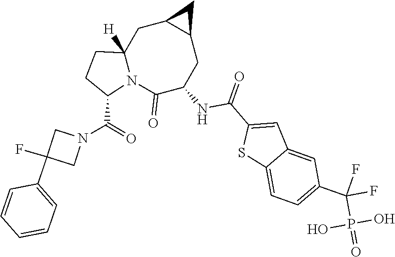 [SMILES: O=C(N[C@H]1C[C@@H]2C[C@@H]2C[C@H]2CC[C@@H](C(=O)N3CC(F)(c4ccccc4)C3)N2C1=O)c1cc2cc(C(F)(F)P(=O)(O)O)ccc2s1] | 662.3 [M + H]⁺ | ¹H NMR (400 MHz, CD₃OD) δ 8.24-8.08 (m, 2H), 7.98-7.86 (m, 1H), 7.74 (d, J = 8.4 Hz, 1H), 7.62-7.50 (m, 2H), 7.50-7.32 (m, 3H), 5.16-5.00 (m, 2H), 4.76- 4.64 (m, 1H), 4.61-4.55 (m, 1H), 4.50-4.27 (m, 3H), 2.53-2.41 (m, 1H), 2.37-2.27 (m, 2H), 2.26- 2.12 (m, 2H), 2.07-1.90 (m, 2H), 1.81-1.69 (m, 1H), 1.48-1.37 (m, 1H), 1.02-0.91 (m, 1H), 0.90- 0.80 (m, 1H), 0.13-0.02 (m, 1H). |
| 65 | (difluoro(2- (((3S,6S,7aS,8aR, 9aR)-5-oxo-3-(3- (pyrimidin-2-yl) azetidine-1-carbonyl) decahydro-1H- cyclopropa[d] pyrrolo[1,2-a]azocin- 6-yl)carbamoyl) benzo[b]thiophen-5- yl)methyl) phosphonic acid | 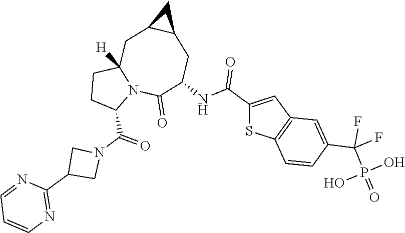 [SMILES: O=C(N[C@H]1C[C@@H]2C[C@@H]2C[C@H]2CC[C@@H](C(=O)N3CC(c4ncccn4)C3)N2C1=O)c1cc2cc(C(F)(F)P(=O)(O)O)ccc2s1] | 646.1 [M + H]⁺ | ¹H NMR (400 MHz, CD₃OD) δ 8.81-8.74 (m, 2H), 8.20-8.08 (m, 2H), 7.92 (t, J = 8.8 Hz, 1H), 7.76- 7.70 (m, 1H), 7.40-7.32 (m, 1H), 5.09-4.98 (m, 2H), 4.59-4.50 (m, 2H), 4.46-4.38 (m, 1H), 4.35- 4.07 (m, 3H), 2.52-2.37 (m, 1H), 2.35-2.25 (m, 2H), 2.24- 2.11 (m, 2H), 2.02-1.90 (m, 2H), 1.80-1.68 (m, 1H), 1.49- 1.38 (m, 1H), 1.02-0.89 (m, 1H), 0.89-0.80 (m, 1H), 0.11- 0.02 (m, 1H) |
| 66 | ((2- (((3S,6S,7aS,8aS, 8bR)-3-((3S,4R or 3R,4S)-3-cyano- 4-phenylpyrrolidine- 1-carbonyl)-5- oxodecahydrocyclo- propa[c]pyrrolo[1,2- a]azepin-6-yl) carbamoyl)benzo[b] thiophen-5-yl) difluoromethyl) phosphonic acid | 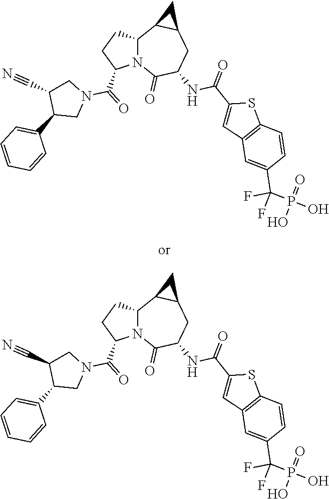 [SMILES: N#C[C@@H]1CN(C(=O)[C@@H]2CC[C@@H]3[C@H]4C[C@H]4C[C@H](NC(=O)c4cc5cc(C(F)(F)P(=O)(O)O)ccc5s4)C(=O)N32)C[C@H]1c1ccccc1 ; N#C[C@H]1CN(C(=O)[C@@H]2CC[C@@H]3[C@H]4C[C@H]4C[C@H](NC(=O)c4cc5cc(C(F)(F)P(=O)(O)O)ccc5s4)C(=O)N32)C[C@@H]1c1ccccc1] | 669.0 [M + H]⁺ | ¹H NMR (500 MHz, DMSO-d₆) δ 8.85-8.62 (m, 1H), 8.33-8.28 (m, 1H), 8.11 (dd, J = 8.5, 3.7 Hz, 1H), 8.06 (s, 1H), 7.56 (d, J = 8.6 Hz, 1H), 7.45-7.35 (m, 4H), 7.33- 7.27 (m, 1H), 4.52-4.45 (m, 2H), 4.27-4.16 (m, 1H), 4.11-3.96 (m, 2H), 3.84-3.78 (m, 2H), 3.30-3.23 (m, 1H), 3.12-3.00 (m, 1H), 2.33- 2.23 (m, 1H), 2.22-2.14 (m, 1H), 2.13-1.94 (m, 4H), 1.08 (m, 2H), 0.84 (m, 1H), 0.56 (m, 1H). |
| | | SFC peak 2 used for biological | | |
| | | testing | | |
| 67 | (difluoro(2- (((3S,6S,7aS,8aS, 8bR)-5-oxo-3-(3- (pyridin-3-yl) azetidine-1- carbonyl)decahydro- cyclopropa[c]pyrrolo [1,2-a]azepin-6-yl) carbamoyl)benzo[b] thiophen-5-yl) methyl)phosphonic acid | 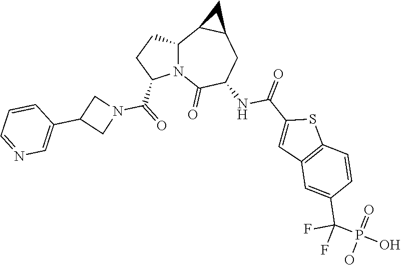 | 631.0 [M + H]⁺ | ¹H NMR (600 MHz, DMSO-d₆) δ 8.88-8.42 (m, 2H), 8.31 (s, 1H), 8.10 (d, J = 8.5 Hz, 1H), 8.07 (d, J = 11.4 Hz, 1H), 7.98-7.80 (m, 1H), 7.57 (d, J = 8.6 Hz, 1H), 7.53-7.00 (m, 1H), 4.83-4.60 (m, 1H), 4.53-4.44 (m, 1H), 4.35 (m, 1H), 4.27-4.18 (m, 2H), 3.88 (m, 2H), 3.84-3.77 (m, 2H), 2.26 (m, 1H), 2.16-1.98 (m, 5H), 1.22-1.05 (m, 2H), 0.85 (m, 1H), 0.57 (m, 1H). |
| 68 | (difluoro(2- (((3S,6S,7aR,8aS, 9aR)-5-oxo-3-(3- (m-tolyl)azetidine- 1-carbonyl) decahydro-1H- cyclopropa[d] pyrrolo[1,2-a]azocin- 6-yl)carbamoyl) benzo[b]thiophen- 5-yl)methyl) phosphonic acid | 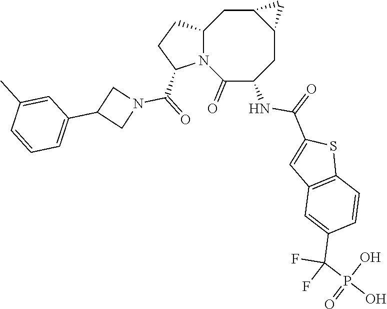 [SMILES: Cc1cccc(C2CN(C(=O)[C@@H]3CC[C@@H]4C[C@@H]5C[C@@H]5C[C@H](NC(=O)c5cc6cc(C(F)(F)P(=O)(O)O)ccc6s5)C(=O)N43)C2)c1] | 658.0 [M + H]⁺ | |
| 69 | (difluoro(2- (((3S,6S,7aR,8aS, 9aR)-3-(3- ((methylsulfonyl) methyl)azetidine-1- carbonyl)-5- oxodecahydro-1H- cyclopropa[d] pyrrolo[1,2-a]azocin- 6-yl)carbamoyl) benzo[b]thiophen-5- yl)methyl) phosphonic acid | 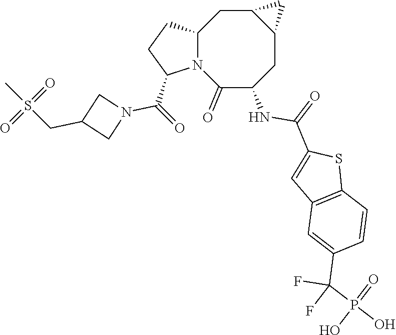 [SMILES: CS(=O)(=O)CC1CN(C(=O)[C@@H]2CC[C@@H]3C[C@@H]4C[C@@H]4C[C@H](NC(=O)c4cc5cc(C(F)(F)P(=O)(O)O)ccc5s4)C(=O)N32)C1] | 660.0 [M + H]⁺ | ¹H NMR (400 MHz, DMSO-d₆) δ 8.83-8.80 (m, 1H), 8.30 (d, J = 2.3 Hz, 1H), 8.11 (d, J = 8.6 Hz, 1H), 8.07 (s, 1H), 7.57 (d, J = 8.6 Hz, 1H), 4.81 (app. t, J = 6.2 Hz, 1H), 4.50 (t, J = 8.6 Hz, 1H), 4.35 (t, J = 8.5 Hz, 1H), 4.24 (m, 2H), 3.53 (d, J = 7.4 Hz, 2H), 3.42 (m, 1H), 3.19-3.01 (m, 3H), 2.23 (m, 1H), 2.12 (d, J = 9.6 Hz, 1H), 2.02 (m, 2H), 1.86 m, 2H), 1.64 (s, 1H), 1.58-1.47 (m, 1H), 1.38-1.27 (m, 1H), 1.26-1.11 (m, 3H), 0.75 (m, 1H), 0.05 (s, 1H). |
| 70 | ((2- (((3S,6S,7aR,8aS, 9aR)-3-(3-cyano-3- (pyridin-2-yl) azetidine-1- carbonyl)-5- oxodecahydro-1H- cyclopropa[d] pyrrolo[1,2- a]azocin-6-yl) carbamoyl)benzo[b] thiophen-5-yl) difluoromethyl) phosphonic acid | 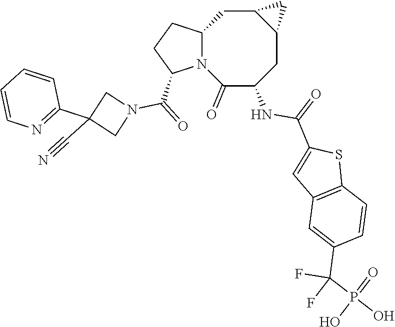 [SMILES: N#CC1(c2ccccn2)CN(C(=O)[C@@H]2CC[C@@H]3C[C@@H]4C[C@@H]4C[C@H](NC(=O)c4cc5cc(C(F)(F)P(=O)(O)O)ccc5s4)C(=O)N32)C1] | 670.0 [M + H]⁺ | ¹H NMR (400 MHz, DMSO-d₆) δ 8.92 (m, 1H), 8.66 (m, 1H), 8.30 (m, 1H), 8.18-8.04 (m, 2H), 7.99- 7.88 (m, 1H), 7.71 (d, J = 7.9 Hz, 1H), 7.59 (d, J = 8.5 Hz, 1H), 7.50-7.38 (m, 1H), 5.13-4.67 (m, 4H), 4.51-4.45 (m, 1H), 4.30 (m, 2H), 2.33-2.13 (m, 2H), 2.07 (m, 2H), 1.93-1.79 (m, 2H), 1.72 (m, 1H), 1.48 (m, 1H), 1.31 (s, 1H), 1.25-1.17 (m, 1H), 0.83- 0.72 (m, 1H), 0.06 (s, 1H). |
| 71 | (difluoro(2- (((3S,6S,7aR,8aS, 9aR)-3-(3- (morpholine-4- carbonyl)azetidine- 1-carbonyl)-5- oxodecahydro-1H- cyclopropa[d] pyrrolo[1,2-a]azocin- 6-yl)carbamoyl) benzo[b]thiophen-5- yl)methyl) phosphonic acid | 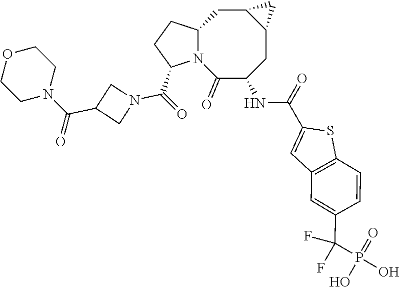 [SMILES: O=C(N[C@H]1C[C@H]2C[C@H]2C[C@H]2CC[C@@H](C(=O)N3CC(C(=O)N4CCOCC4)C3)N2C1=O)c1cc2cc(C(F)(F)P(=O)(O)O)ccc2s1] | 681.0 [M + H]⁺ | ¹H NMR (400 MHz, DMSO-d₆) δ 8.82 (dd, J = 14.3, 7.4 Hz, 1H), 8.28 (app. s, 1H), 8.08 (m, 2H), 7.73 (br. s, 1H), 4.80 (m, 1H), 4.45 (m, 1H), 4.26 (m, 2H), 4.16 (d, J = 6.8 Hz, 1H), 4.04-3.97 (m, 2H), 3.54-3.50 (m, 4H), 3.45 (m, 2H), 3.25 (m, 2H), 2.26-2.11 (m, 2H), 2.03 (m, 2H), 1.85 (m, 2H), 1.62 (s, 1H), 1.50 (m, 1H), 1.32 (m, 1H), 1.16 (m, 2H), 0.76 (m, 1H), 0.03 (s, 1H). |
| 72 | ((2- (((3S,6S,7aR,8aS, 9aR)-3-(3-((N,S- dimethyl- sulfonimidoyl) methyl)azetidine-1- carbonyl)-5- oxodecahydro-1H- cyclopropa[d] pyrrolo[1,2-a]azocin- 6-yl)carbamoyl) benzo[b]thiophen-5- yl)difluoromethyl) phosphonic acid | 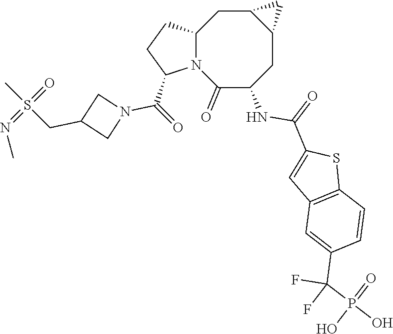 [SMILES: CN=S(C)(=O)CC1CN(C(=O)[C@@H]2CC[C@@H]3C[C@@H]4C[C@@H]4C[C@H](NC(=O)c4cc5cc(C(F)(F)P(=O)(O)O)ccc5s4)C(=O)N32)C1] | 673.3 [M + H]⁺ | ¹H NMR (600 MHz, DMSO-d₆) δ 8.80 (d, J = 7.1 Hz, 1H), 8.29 (d, J = 8.2 Hz, 1H), 8.10 (d, J = 8.5 Hz, 1H), 8.06 (s, 1H), 7.57 (d, J = 8.6 Hz, 1H), 4.80 (m, 1H), 4.49- 4.31 (m, 1H), 4.23 (m, 1H), 4.15 (m, 1H), 3.99 (m, 2H), 3.94 (m, 1H), 3.16-3.05 (m, 2H), 3.02 (m, 3H), 2.61 (m, 3H), 2.22 (m, 1H), 2.12 (s, 1H), 2.01 (m, 2H), 1.85 (m, 2H), 1.63 (m, 1H), 1.54-1.44 (m, 1H), 1.30 (m, 1H), 1.22-1.16 (m, 1H), 0.75 (m, 1H), 0.03 (s, 1H). |
| 73 | ((2- (((3S,6S,7aR,8aS, 9aR)-3-(3-(2,2- difluoroethyl)-3- phenylazetidine-1- carbonyl)-5- oxodecahydro-1H- cyclopropa[d] pyrrolo[1,2-a]azocin- 6-yl)carbamoyl) benzo[b]thiophen-5- yl)difluoromethyl) phosphonic acid | 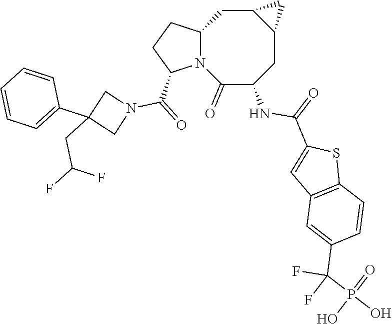 [SMILES: O=C(N[C@H]1C[C@H]2C[C@H]2C[C@H]2CC[C@@H](C(=O)N3CC(CC(F)F)(c4ccccc4)C3)N2C1=O)c1cc2cc(C(F)(F)P(=O)(O)O)ccc2s1] | 708.4 [M + H]⁺ | ¹H NMR (600 MHz, DMSO-d₆) δ 8.90-8.78 (m, 1H), 8.29-8.20 (m, 1H), 8.13-8.00 (m, 2H), 7.57 (dd, J = 12.7, 8.6 Hz, 1H), 7.36 (m, 2H), 7.31-7.27 (m, 1H), 7.24 (t, J = 6.7 Hz, 2H), 5.79-5.49 (m, 1H), 4.80 (m, 1H), 4.57 (m, 1H), 4.43 (m, 1H), 4.28 (m, 1H), 4.19- 4.13 (m, 1H), 4.11-4.01 (m, 2H), 2.25 (m, 1H), 2.21-2.09 (m, 2H), 2.06-1.98 (m, 2H), 1.86 (m, 2H), 1.77-1.64 (m, 1H), 1.54 (m, 1H), 1.53-1.39 (m, 1H), 1.29 (s, 1H), 1.22-1.15 (m, 1H), 0.74 (m, 1H), 0.01 (s, 1H). |
| 74 | ((2- (((3S,6S,7aR,8aS, 9aR)-3-(3-(4- cyanophenyl) azetidine-1- carbonyl)-5- oxodecahydro-1H- cyclopropa[d] pyrrolo[1,2-a]azocin- 6-yl)carbamoyl) benzo[b]thiophen-5- yl)difluoromethyl) phosphonic acid | 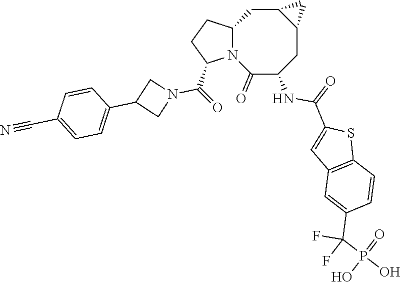 [SMILES: N#Cc1ccc(C2CN(C(=O)[C@@H]3CC[C@@H]4C[C@@H]5C[C@@H]5C[C@H](NC(=O)c5cc6cc(C(F)(F)P(=O)(O)O)ccc6s5)C(=O)N43)C2)cc1] | 669.0 [M + H]⁺ | ¹H NMR (600 MHz, DMSO-d₆) δ 8.88-8.82 (m, 1H), 8.31 (d, J = 8.6 Hz, 1H), 8.12 (d, J = 8.7 Hz, 1H), 8.08 (s, 1H), 7.82 (app. t, J = 8.6 Hz, 2H), 7.58 (d, J = 8.3 Hz, 2H), 7.53 (d, J = 8.0 Hz, 1H), 4.86- 4.80 (m, 1H), 4.79-4.58 (m, 1H), 4.37-4.22 (m, 3H), 4.18 (m, 1H), 4.00 (s, 2H), 3.82 (m, 1H), 2.23 (m, 1H), 2.18 (dt, J = 13.4, 7.2 Hz, 1H), 2.03 (m, 2H), 1.87 (m, 2H), 1.67 (m, 1H), 1.52 (m, 1H), 1.31 (s, 1H), 1.18 (m, 1H), 0.75 (m, 1H), 0.03 (br. s, 1H). |
| 75 | (difluoro(2- (((3S,6S,7aR,8aS, 9aR)-3-(3- methoxy-3-(1- methyl-1H-imidazol- 2-yl)azetidine-1- carbonyl)-5- oxodecahydro-1H- cyclopropa[d] pyrrolo[1,2-a]azocin- 6-yl)carbamoyl) benzo[b]thiophen-5- yl)methyl) phosphonic acid | 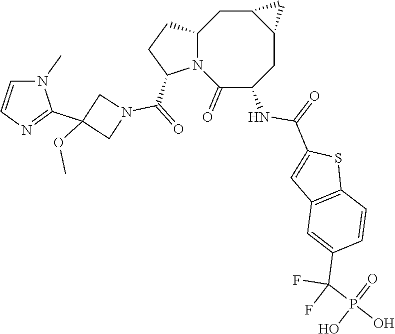 [SMILES: COC1(c2nccn2C)CN(C(=O)[C@@H]2CC[C@@H]3C[C@@H]4C[C@@H]4C[C@H](NC(=O)c4cc5cc(C(F)(F)P(=O)(O)O)ccc5s4)C(=O)N32)C1] | 678.4 [M + H]⁺ | ¹H NMR (600 MHz, DMSO-d₆) δ 8.88-8.80 (m, 1H), 8.32-8.28 (m, 1H), 8.10 (dd, J = 12.9, 8.3 Hz, 1H), 8.05 (d, J = 19.1 Hz, 1H), 7.57 (t, J = 8.8 Hz, 1H), 7.25 (d, J = 24.4 Hz, 1H), 6.90 (d, J = 14.7 Hz, 1H), 4.83-4.74 (m, 2H), 4.44- 4.41 (m, 1H), 4.37-4.27 (m, 2H), 4.16 (m, 1H), 4.02 (m, 1H), 3.56 (app. m, 3H), 2.90 (app. m, 3H), 2.25-2.10 (m, 2H), 2.06-1.97 (m, 2H), 1.85 (m, 2H), 1.68-1.56 (m, 1H), 1.50 (m, 1H), 1.30 (s, 1H), 1.20 (m, 1H), 0.75 (m, 1H), 0.04 (br. s, 1H). |
| 76 | (difluoro(2- (((3S,6S,7aR,8aS, 9aR)-3-(3-(4-methyl- 4H-1,2,4-triazol-3- yl)azetidine-1- carbonyl)-5- oxodecahydro-1H- cyclopropa[d] pyrrolo[1,2-a]azocin- 6-yl)carbamoyl) benzo[b]thiophen-5- yl)methyl) phosphonic acid | 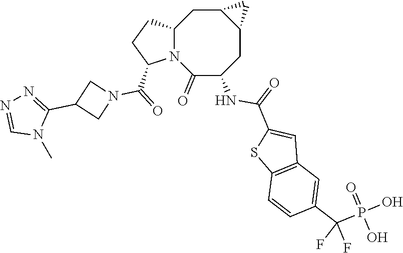 [SMILES: Cn1cnnc1C1CN(C(=O)[C@@H]2CC[C@@H]3C[C@@H]4C[C@@H]4C[C@H](NC(=O)c4cc5cc(C(F)(F)P(=O)(O)O)ccc5s4)C(=O)N32)C1] | 649.2 [M + H]⁺ | ¹H NMR (500 MHz, DMSO-d₆) δ 8.87-8.81 (m, 1H), 8.40 (d, J = 10.7 Hz, 1H), 8.29 (d, J = 11.0 Hz, 1H), 8.13-8.04 (m, 2H), 7.56 (m, 1H), 4.81 (m, 1H), 4.58-4.40 (m, 2H), 4.26 (m, 2H), 4.17 (m, 2H), 4.04-4.00 (m, 1H), 3.51 (s, 3H), 2.23-2.13 (m, 2H), 2.03 (m, 2H), 1.87 (ddt, J = 15.2, 11.2, 5.6 Hz, 2H), 1.69-1.62 (m, 1H), 1.55-1.47 (m, 1H), 1.35-1.28 (m, 1H), 1.18 (s, 1H), 0.75 (m, 1H), 0.03 (br. s, 1H). |
| 77 | (difluoro(2- (((3S,6S,7aR,8aS, 9aR)-5-oxo-3-(3- (2,4,5,6-tetrahydro- cyclopenta[c] pyrazol-3-yl) azetidine-1-carbonyl) decahydro-1H- cyclopropa[d] pyrrolo[1,2-a]azocin- 6-yl)carbamoyl) benzo[b]thiophen-5- yl)methyl) phosphonic acid | 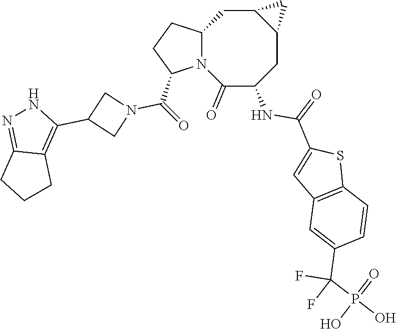 [SMILES: O=C(N[C@H]1C[C@H]2C[C@H]2C[C@H]2CC[C@@H](C(=O)N3CC(c4[nH]nc5c4CCC5)C3)N2C1=O)c1cc2cc(C(F)(F)P(=O)(O)O)ccc2s1] | 674.4 [M + H]⁺ | ¹H NMR (600 MHz, DMSO-d₆) δ 8.84-8.61 (m, 1H), 8.28-8.23 (m, 1H), 8.12 (dd, J = 8.7, 2.7 Hz, 1H), 8.07 (br. s, 1H), 7.57 (d, J = 8.7 Hz, 1H), 4.80 (m, 1H), 4.56 (m, 1H), 4.42-4.22 (m, 2H), 4.20- 4.14 (m, 2H), 3.96-3.84 (m, 1H), 3.80 (m, 1H), 3.74 (m, 1H), 2.57- 2.53 (m, 4H), 2.38 (s, 1H), 2.30- 2.14 (m, 2H), 2.01 (m, 2H), 1.94- 1.76 (m, 2H), 1.70-1.60 (m, 1H), 1.58-1.40 (m, 1H), 1.31 (s, 1H), 1.17 (m, 1H), 0.79-0.72 (m, 1H), 0.03 (br. s, 1H). |
| 78 | ((2- (((3S,6S,7aR,8aS, 9aR)-3-(3-(1H- benzo[d]imidazol-2- yl)azetidine-1- carbonyl)-5- oxodecahydro-1H- cyclopropa[d] pyrrolo[1,2-a]azocin- 6-yl)carbamoyl) benzo[b]thiophen-5- yl)difluoromethyl) phosphonic acid | 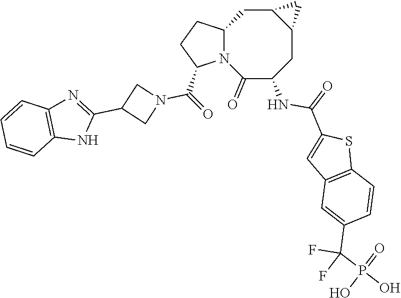 [SMILES: O=C(N[C@H]1C[C@H]2C[C@H]2C[C@H]2CC[C@@H](C(=O)N3CC(c4nc5ccccc5[nH]4)C3)N2C1=O)c1cc2cc(C(F)(F)P(=O)(O)O)ccc2s1] | 684.2 [M + H]⁺ | ¹H NMR (500 MHz, DMSO-d₆) δ 8.81 (m, 1H), 8.30-8.28 (m, 1H), 8.14-8.02 (m, 3H), 7.60-7.52 (m, 2H), 7.48 (m, 1H), 7.20 (m, 1H), 7.12 (m, 1H), 4.82 (m, 1H), 4.79- 4.42 (m, 3H), 4.30 (m, 2H), 4.20-4.15 (m, 2H), 4.14-4.11 (m, 1H), 2.24-2.15 (m, 2H), 2.05 (m, 2H), 1.91-1.83 (m, 2H), 1.74- 1.66 (m, 1H), 1.51 (m, 1H), 1.33 (m, 1H), 1.20 (m, 1H), 0.75 (m, 1H), 0.04 (br. s, 1H). |
| 79 | (difluoro(2- (((3S,6S,7aR,8aS, 9aR)-3-(3-isopropyl- 3-phenylazetidine- 1-carbonyl)-5- oxodecahydro-1H- cyclopropa[d] pyrrolo[1,2-a]azocin- 6-yl)carbamoyl) benzo[b]thiophen-5- yl)methyl) phosphonic acid | 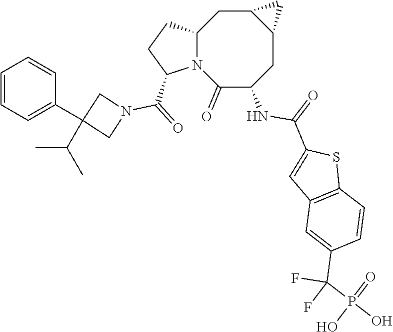 [SMILES: CC(C)C1(c2ccccc2)CN(C(=O)[C@@H]2CC[C@@H]3C[C@@H]4C[C@@H]4C[C@H](NC(=O)c4cc5cc(C(F)(F)P(=O)(O)O)ccc5s4)C(=O)N32)C1] | 686.4 [M + H]⁺ | ¹H NMR (400 MHz, DMSO-d₆) δ 8.89-8.79 (m, 1H), 8.26 (m, 1H), 8.16-8.02 (m, 2H), 7.56 (m, 1H), 7.32 (m, 2H), 7.23 (m, 1H), 7.11 (m, 2H), 4.80 (s, 1H), 4.50-4.28 (m, 4H), 4.16 (s, 2H), 3.96 (m, 2H), 3.16 (s, 1H), 2.06 (m, 4H), 1.87 (s, 1H) 1.56 (m, 2H), 1.30 (s, 1H), 1.19 (s, 1H), 0.77-0.69 (m, 6H), 0.01 (br. s, 1H). |
| 80 | (difluoro(2- (((3S,6S,7aR,8aS, 9aR)-5-oxo-3-(3- (pyrimidin-2-yl) azetidine-1- carbonyl)decahydro- 1H-cyclopropa[d] pyrrolo[1,2-a]azocin- 6-yl)carbamoyl) benzo[b]thiophen-5- yl)methyl) phosphonic acid | 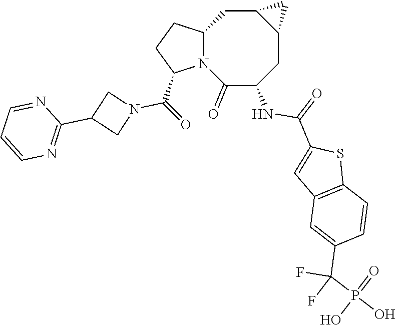 [SMILES: O=C(N[C@H]1C[C@H]2C[C@H]2C[C@H]2CC[C@@H](C(=O)N3CC(c4ncccn4)C3)N2C1=O)c1cc2cc(C(F)(F)P(=O)(O)O)ccc2s1] | 646.4 [M + H]⁺ | ¹H NMR (600 MHz, DMSO-d₆) δ 8.85-8.77 (m, 3H), 8.32-8.27 (m, 1H), 8.13-8.05 (m, 2H), 7.57 (m, 1H), 7.42-7.37 (m, 1H), 4.82- 4.80 (m, 1H), 4.55 (m, 1H), 4.31 (m, 1H), 4.18-4.12 (m, 2H), 4.06- 4.00 (m, 1H), 3.06 (m, 1H), 2.23- 2.12 (m, 2H), 2.03 (m, 2H), 1.88 (m, 2H), 1.65 (m, 1H), 1.53 (m, 1H), 1.31 (s, 1H), 1.20 (m, 1H), 1.15 (m, 1H), 0.75 (m, 1H), 0.02 (br. s, 1H). |
| 81 | (difluoro(2- (((3S,6S,7aR,8aS, 9aR)-3-(3-(5- methyl-4H-1,2,4- triazol-3-yl) azetidine-1- carbonyl)-5- oxodecahydro-1H- cyclopropa[d] pyrrolo[1,2-a]azocin- 6-yl)carbamoyl) benzo[b]thiophen-5- yl)methyl) phosphonic acid | 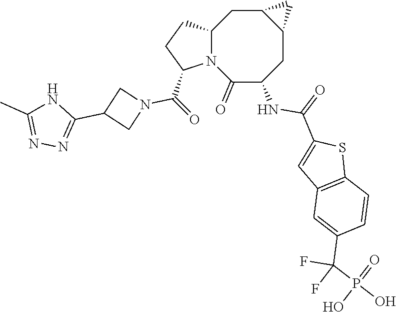 [SMILES: Cc1nnc(C2CN(C(=O)[C@@H]3CC[C@@H]4C[C@@H]5C[C@@H]5C[C@H](NC(=O)c5cc6cc(C(F)(F)P(=O)(O)O)ccc6s5)C(=O)N43)C2)[nH]1] | 649.2 [M + H]⁺ | ¹H NMR (500 MHz, DMSO-d₆) δ 8.84 (d, J = 7.6 Hz, 1H), 8.31 (d, J = 6.8 Hz, 1H), 8.12 (dd, J = 8.6, 3.3 Hz, 1H), 8.07 (d, J = 6.5 Hz, 1H), 7.57 (d, J = 8.6 Hz, 1H), 4.81 (s, 1H), 4.70-4.40 (m, 2H), 4.28 (m, 2H), 4.19-4.13 (m, 2H), 4.09 (m, 1H), 3.99 (m, 1H), 2.28 (m, 3H), 2.18 (m, 3H), 2.02 (m, 2H), 1.86 (m, 2H), 1.68-1.60 (m, 1H), 1.53 (m, 1H), 1.35-1.28 (m, 1H), 1.19 (s, 1H), 0.75 (m, 1H), 0.04 (s, 1H). |
| 82 | (difluoro(2- (((3S,6S,7aR,8aS, 9aR)-3-(3-methyl-3- (5-methyl-1,3,4- oxadiazol-2-yl) azetidine-1- carbonyl)-5- oxodecahydro-1H- cyclopropa[d] pyrrolo[1,2-a]azocin- 6-yl)carbamoyl) benzo[b]thiophen-5- yl)methyl) phosphonic acid | 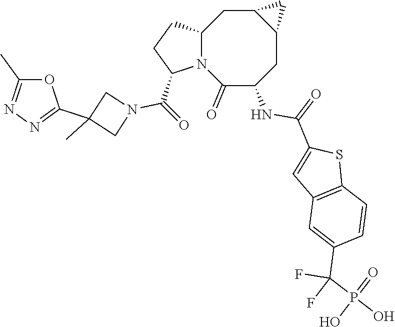 [SMILES: Cc1nnc(C2(C)CN(C(=O)[C@@H]3CC[C@@H]4C[C@@H]5C[C@@H]5C[C@H](NC(=O)c5cc6cc(C(F)(F)P(=O)(O)O)ccc6s5)C(=O)N43)C2)o1] | 664.2 [M + H]⁺ | ¹H NMR (500 MHz, DMSO-d₆) δ 9.79-8.54 (m, 1H), 8.29 (m, 1H), 8.16-8.02 (m, 2H), 7.57 (m, 1H), 4.81 (m, 1H), 4.63-4.48 (m, 1H), 4.40-4.21 (m, 2H), 4.16 (m, 2H), 3.87 (m, 1H), 3.79 (m, 1H), 3.08- 3.02 (m, 1H), 2.27-2.14 (m, 2H), 2.07-1.99 (m, 2H), 1.85 (m, 2H), 1.65 (m, 3H), 1.48 (m, 2H), 1.30 (m, 1H), 1.23-0.96 (m, 2H), 0.75 (m, 1H), 0.02 (s, 1H). |
| 83 | (difluoro(2- (((3S,6S,7aR,8aS, 9aR)-3-(3-((S- methysulfonimidoyl) methyl)azetidine-1- carbonyl)-5- oxodecahydro-1H- cyclopropa[d] pyrrolo[1,2-a]azocin- 6-yl)carbamoyl) benzo[b]thiophen-5- yl)methyl) phosphonic acid | 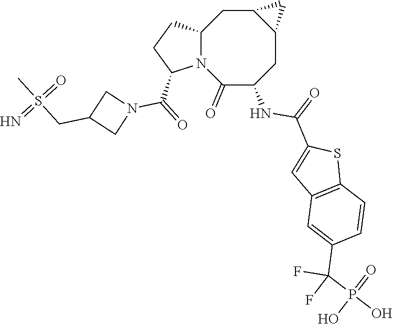 [SMILES: CS(=N)(=O)CC1CN(C(=O)[C@@H]2CC[C@@H]3C[C@@H]4C[C@@H]4C[C@H](NC(=O)c4cc5cc(C(F)(F)P(=O)(O)O)ccc5s4)C(=O)N32)C1] | 659.4 [M + H]⁺ | |
| 84 | (difluoro(2- (((3S,6S,7aR,8aS, 9aR)-3-(3- (methoxymethyl)- 3-phenylazetidine-1- carbonyl)-5- oxodecahydro-1H- cyclopropa[d] pyrrolo[1,2-a]azocin- 6-yl)carbamoyl) benzo[b]thiophen-5- yl)methyl) phosphonic acid | 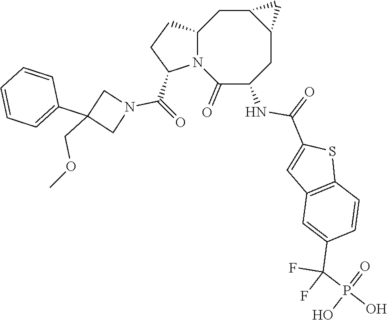 [SMILES: COCC1(c2ccccc2)CN(C(=O)[C@@H]2CC[C@@H]3C[C@@H]4C[C@@H]4C[C@H](NC(=O)c4cc5cc(C(F)(F)P(=O)(O)O)ccc5s4)C(=O)N32)C1] | 688.4 [M + H]⁺ | ¹H NMR (400 MHz, DMSO-d₆) δ 8.89-8.82 (m, 1H), 8.32-8.04 (m, 3H), 7.62-6.90 (m, 6H), 4.81 (s, 1H), 4.51-4.48 (m, 1H), 4.36- 4.27 (m, 2H), 4.16 (s, 1H), 4.04- 3.94 (m, 2H), 3.25 (s, 2H), 3.17 (d, J = 8.6 Hz, 3H), 2.16 (m, 2H), 2.02 (s, 2H), 1.87 (s, 2H), 1.75-1.41 (m, 2H), 1.24 (m, 2H), 0.88-0.59 (m, 1H), 0.03 (s, 1H). |
| 85 | (difluoro(2- (((3S,6S,7aS,8aR, 9aR)-5-oxo-3-(3- (o-tolyl)azetidine-1- carbonyl)decahydro- 1H-cyclopropa[d] pyrrolo[1,2-a]azocin- 6-yl)carbamoyl) benzo[b]thiophen-5- yl)methyl) phosphonic acid | 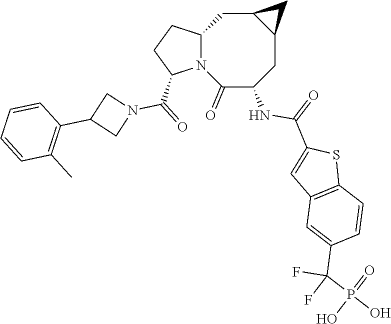 [SMILES: Cc1ccccc1C1CN(C(=O)[C@@H]2CC[C@@H]3C[C@H]4C[C@H]4C[C@H](NC(=O)c4cc5cc(C(F)(F)P(=O)(O)O)ccc5s4)C(=O)N32)C1] | 658.2 [M + H]⁺ | ¹H NMR (500 MHz, DMSO-d₆) δ 8.89 (m, 1H), 8.33-8.31 (m, 1H), 8.11 (t, J = 8.0 Hz, 1H), 8.06 (d, J = 11.5 Hz, 1H), 7.68-7.48 (m, 1H), 7.35 (m, 1H), 7.24-7.05 (m, 3H), 4.96-4.85 (m, 1H), 4.61-4.47 (m, 1H), 4.37 (dd, J = 8.2, 3.7 Hz, 1H), 4.17 (m, 2H), 4.02 (m, 1H), 3.97-3.80 (m, 2H), 2.28 (m, 1H), 2.15 (s, 3H), 2.13-1.90 (m, 4H), 1.88-1.77 (m, 2H), 1.67-1.56 (m, 1H), 1.35 (s, 1H), 0.77 (m, 1H), 0.70 (m, 1H), −0.02 (br. s, 1H). |
| 86 | (difluoro(2- (((3S,6S,7aS,8aR, 9aR)-5-oxo-3-(3-(3- (pyridin-2-yl) azetidine-1-carbonyl) azetidine-1- carbonyl)decahydro- 1H-cyclopropa[d] pyrrolo[1,2-a]azocin- 6-yl)carbamoyl) benzo[b]thiophen-5- yl)methyl) phosphonic acid | 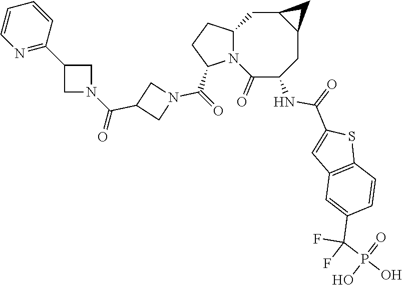 [SMILES: O=C(N[C@H]1C[C@@H]2C[C@@H]2C[C@H]2CC[C@@H](C(=O)N3CC(C(=O)N4CC(c5ccccn5)C4)C3)N2C1=O)c1cc2cc(C(F)(F)P(=O)(O)O)ccc2s1] | 728.2 [M + H]⁺ | ¹H NMR (400 MHz, DMSO-d₆) δ 8.93-8.85 (m, 1H), 8.61-8.54 (m, 1H), 8.36-8.33 (m, 1H), 8.14 (s, 1H), 8.08 (m, 1H), 7.83 (m, 1H), 7.59 (m, 1H), 7.42 (m, 1H), 7.35 (m, 1H), 4.94 (m, 1H), 4.61 (m, 1H), 4.51 (m, 1H), 4.42 (m, 1H), 4.32 (m, 2H), 4.23 (m, 2H), 4.16 (m, 1H), 4.01 (m, 1H), 3.84 (m, 2H), 2.33 (m, 2H), 2.06 (m, 4H), 1.84 (m, 2H), 1.63 (m, 1H), 1.35 (m, 1H), 0.79 (m, 1H), 0.71 (m, 1H), −0.01 (br. s, 1H). |
| 87 | (difluoro(2- (((3S,6S,7aS,8aR, 9aR)-3-(3-hydroxy- 3-(pyridin-2-yl) azetidine-1- carbonyl)-5- oxodecahydro-1H- cyclopropa[d] pyrrolo[1,2-a]azocin- 6-yl)carbamoyl) benzo[b]thiophen-5- yl)methyl) phosphonic acid | 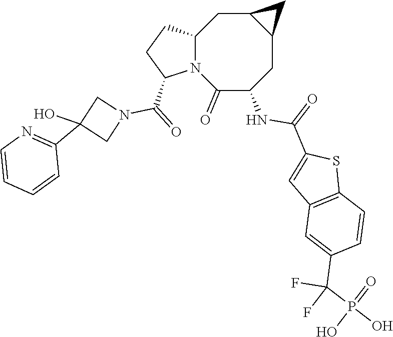 [SMILES: O=C(N[C@H]1C[C@@H]2C[C@@H]2C[C@H]2CC[C@@H](C(=O)N3CC(O)(c4ccccn4)C3)N2C1=O)c1cc2cc(C(F)(F)P(=O)(O)O)ccc2s1] | 661.0 [M + H]⁺ | ¹H NMR (600 MHz, DMSO-d₆) δ 8.87 (d, J = 7.4 Hz, 1H), 8.58 (dd, J = 18.2, 4.8 Hz, 1H), 8.32 (d, J = 13.6 Hz, 1H), 8.11 (dd, J = 8.5, 5.9 Hz, 1H), 8.06 (d, J = 10.4 Hz, 1H), 7.85-7.77 (m, 1H), 7.65- 7.59 (m, 1H), 7.57 (dd, J = 9.1, 4.1 Hz, 1H), 7.31-7.29 (m, 1H), 6.56 (s, 1H), 4.92 (p, J = 8.2 Hz, 1H), 4.55-4.48 (m, 1H), 4.41 (dd, J = 8.2, 3.3 Hz, 1H), 4.29 (d, J = 9.9 Hz, 1H), 4.19 (d, J = 8.8 Hz, 1H), 4.14 (m, 2H), 2.33 (m, 1H), 2.18-1.98 (m, 4H), 1.93-1.81 (m, 2H), 1.63 (m, 1H), 1.38 (m, 1H), 0.80-0.71 (m, 2H), −0.01 (br. s, 1H). |
| 88 | (difluoro(2- (((3S,6S,7aS,8aR, 9aR)-3-(3-(6- methoxypyridin-3- yl)pyrrolidine-1- carbonyl)-5- oxodecahydro-1H- cyclopropa[d] pyrrolo[1,2-a]azocin- 6-yl)carbamoyl) benzo[b]thiophen-5- yl)methyl) phosphonic acid | 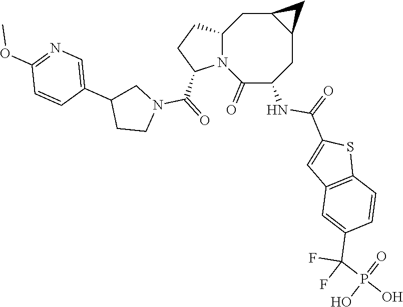 [SMILES: COc1ccc(C2CCN(C(=O)[C@@H]3CC[C@@H]4C[C@H]5C[C@H]5C[C@H](NC(=O)c5cc6cc(C(F)(F)P(=O)(O)O)ccc6s5)C(=O)N43)C2)cn1] | 689.0 [M + H]⁺ | ¹H NMR (500 MHz, DMSO-d₆) δ 8.84 (br. s, 1H), 8.31 (br. s, 1H), 8.09 (m, 3H), 7.70-7.61 (m, 1H), 7.61-7.53 (m, 1H), 6.77-6.73 (m, 1H), 4.93 (m, 1H), 4.58 (m, 1H), 4.20 (m, 2H), 4.01-3.92 (m, 1H), 3.85-3.73 (m, 4H), 3.56 (m, 2H), 3.31-3.14 (m, 2H), 2.15 (m, 2H), 2.04 (m, 3H), 1.85 (m, 2H), 1.61 (m, 1H), 1.44 (s, 1H), 0.83 (m, 1H), 0.74-0.61 (m, 1H), −0.01 (br. s, 1H). |
| 89 | ((2- (((3S,6S,7aS,8aR, 9aR)-3-(3-cyano-3- (pyridin-2-yl) azetidine-1- carbonyl)-5- oxodecahydro-1H- cyclopropa[d] pyrrolo[1,2-a]azocin- 6-yl)carbamoyl) benzo[b]thiophen-5- yl)difluoromethyl) phosphonic acid | 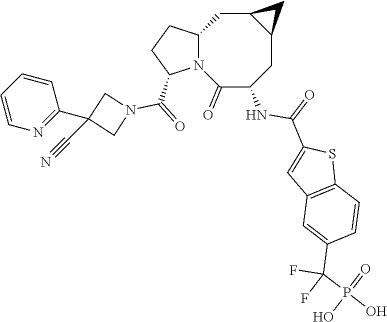 [SMILES: N#CC1(c2ccccn2)CN(C(=O)[C@@H]2CC[C@@H]3C[C@H]4C[C@H]4C[C@H](NC(=O)c4cc5cc(C(F)(F)P(=O)(O)O)ccc5s4)C(=O)N32)C1] | 670.0 [M + H]⁺ | |
| 90 | (difluoro(2- (((3S,6S,7aS,8aR, 9aR)-5-oxo-3-(7′- oxo-6′,7′-dihydro- spiro[azetidine- 3,5′-pyrrolo[1,2- a]imidazole]-1- carbonyl)decahydro- 1H-cyclopropa[d] pyrrolo[1,2-a]azocin- 6-yl)carbamoyl) benzo[b]thiophen-5- yl)methyl) phosphonic acid | 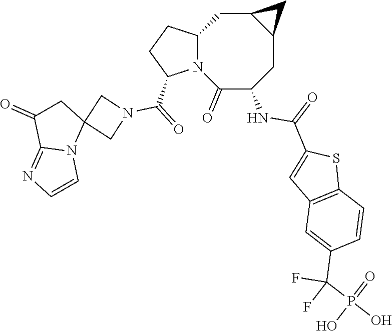 [SMILES: O=C(N[C@H]1C[C@@H]2C[C@@H]2C[C@H]2CC[C@@H](C(=O)N3CC4(CC(=O)c5nccn54)C3)N2C1=O)c1cc2cc(C(F)(F)P(=O)(O)O)ccc2s1] | 674.0 [M + H]⁺ | ¹H NMR (400 MHz, DMSO-d₆) δ 8.97-8.80 (m, 1H), 8.35-8.30 (m, 1H), 8.14-8.00 (m, 3H), 7.61- 7.45 (m, 2H), 4.97-4.85 (m, 2H), 4.60 (dd, J = 28.8, 9.9 Hz, 1H), 4.38-4.17 (m, 4H), 3.48-3.42 (m, 2H), 2.19-1.97 (m, 5H), 1.86 (m, 2H), 1.66 (br. s, 1H), 1.44- 1.06 (m, 2H), 0.92-0.71 (m, 2H). |

### Prepared According to General Scheme 2: Phosphonate Acid

The following compounds in Table 27 were prepared according to the representative procedure described above for the synthesis of ((2-(((3S,6S,7aS,8aR,9aR)-3-([11,1′-biphenyl]-4-ylcarbamoyl)-5-oxodecahydro-1H-cyclopropa[d]pyrrolo[1,2-a]azocin-6-yl)carbamoyl)benzo[b]thiophen-5-yl)difluoromethyl)phosphonic acid (Example 3) utilizing the appropriate starting materials and modifications.

TABLE 27

| Com- | | | | |
|---|---|---|---|---|
| pound | Name | Structure | LCMS | NMR |
| 91 | (difluoro(2- (((3S,6S,7aS, 8aR,9aR)-3- (3- morpholino- azetidine-1- carbonyl)-5- oxodecahydro- 1H- cyclopropa[d] pyrrolo[1,2- a]azocin-6- yl)carbamoyl )benzo[b]thio- phen-5- yl)methyl) phosphonic acid | 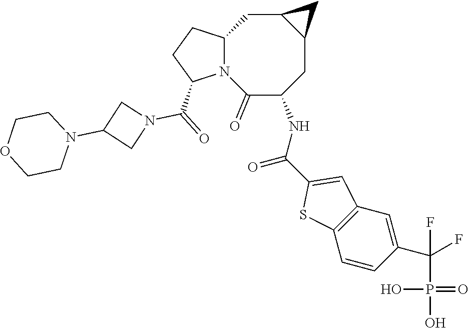 [SMILES: O=C(N[C@H]1C[C@@H]2C[C@@H]2C[C@H]2CC[C@@H](C(=O)N3CC(N4CCOCC4)C3)N2C1=O)c1cc2cc(C(F)(F)P(=O)(O)O)ccc2s1] | 653.2 [M + H]⁺ | ¹H NMR (400 MHZ, CD₃OD) δ 8.19-8.09 (m, 2H), 7.99-7.92 (m, 1H), 7.72 (d, J = 8.4 Hz, 1H), 5.00 (t, J = 9.2 Hz, 1H), 4.80-4.71 (m, 1H), 4.59-4.48 (m, 1H), 4.44 (s, 1H), 4.30 (dd, J = 7.2, 11.2 Hz, 2H), 4.24-4.20 (m, 1H), 4.19-4.05 (m, 2H), 3.90 (s, 4H), 3.27- 3.12 (m, 3H), 2.54- 2.40 (m, 1H), 2.35- 2.08 (m, 4H), 2.04- 1.92 (m, 2H), 1.82- 1.66 (m, 1H), 1.44- 1.29 (m, 1H), 0.97- 0.81 (m, 2H), 0.13- 0.03 (m, 1H). |
| 92 | ((2- (((35,6S,7aS, 8aR,9aR)-3- (6-acetyl-2,6- diazaspiro[3. 4]octane-2- carbonyl)-5- oxodeca- hydro-1H- cyclopropa[d] pyrrolo[1,2- a]azocin-6- yl)carbamoyl )benzo[b]thio- phen-5- yl)difluoro- methyl)phos- phonic acid | 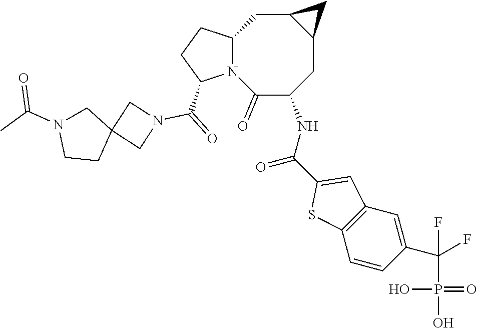 [SMILES: CC(=O)N1CCC2(C1)CN(C(=O)[C@@H]1CC[C@@H]3C[C@H]4C[C@H]4C[C@H](NC(=O)c4cc5cc(C(F)(F)P(=O)(O)O)ccc5s4)C(=O)N31)C2] | 665.2 [M + H]⁺ | ¹H NMR (400 MHZ, CD₃OD) δ 8.21-8.10 (m, 2H), 7.93 (d, J = 8.0 Hz, 1H), 7.74 (d, J = 8.4 Hz, 1H), 5.07- 5.00 (m, 1H), 4.50 (d, J = 5.6 Hz, 2H), 4.31- 4.16 (m, 2H), 3.99- 3.83 (m, 2H), 3.78- 3.68 (m, 1H), 3.66- 3.54 (m, 2H), 3.49- 3.43 (m, 1H), 2.52- 2.40 (m, 1H), 2.34- 2.12 (m, 6H), 2.06- 2.01 (m, 3H), 2.00- 1.91 (m, 2H), 1.80- 1.68 (m, 1H), 1.48- 1.36 (m, 1H), 1.00- 0.90 (m, 1H), 0.84 (d, J = 4.4 Hz, 1H), 0.11- 0.03 (m, 1H) |
| 93 | (difluoro(2- (((3S,6S,7aS, 8aR,9aR)-3- (3-methyl-3- (morpholine- 4- carbonyl)aze- tidine-1- carbonyl)-5- oxodecahydro- 1H- cyclopropa[d] pyrrolo[1,2- a]azocin-6- yl)carbamoyl) benzo[b]thio- phen-5- yl)methyl) phosphonic acid | 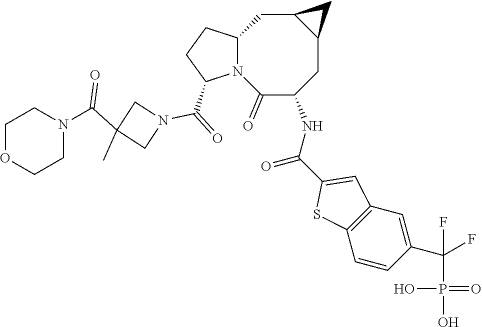 [SMILES: CC1(C(=O)N2CCOCC2)CN(C(=O)[C@@H]2CC[C@@H]3C[C@H]4C[C@H]4C[C@H](NC(=O)c4cc5cc(C(F)(F)P(=O)(O)O)ccc5s4)C(=O)N32)C1] | 695.3 [M + H]⁺ | ¹H NMR (400 MHZ, CD₃OD) δ 8.19 (s, 1H), 8.13 (d, J = 3.2 Hz, 1H), 7.95-7.90 (m, 1H), 7.78-7.72 (m, 1H), 5.06-4.98 (m, 1H), 4.59-4.53 (m, 1H), 4.52-4.45 (m, 1H), 4.42-4.37 (m, 1H), 4.35-4.21 (m, 2H), 4.08-3.80 (m, 1H), 3.70-3.57 (m, 6H), 3.29-3.20 (m, 2H), 2.45 (dd, J = 8.4, 14.8 Hz, 1H), 2.35- 2.07 (m, 4H), 2.02- 1.90 (m, 2H), 1.83- 1.67 (m, 1H), 1.60 (d, J = 1.6 Hz, 3H), 1.51- 1.35 (m, 1H), 1.05- 0.89 (m, 1H), 0.88- 0.75 (m, 1H), 0.15- 0.01 (m, 1H) |
| 94 | (((2- (((3S,6S,7aS, 8aR,9aR)-3- (3-(1H- imidazol-1- yl)azetidine- 1-carbonyl)- 5- oxodecahydro- 1H- cyclopropa[d] pyrrolo[1,2- a]azocin-6- yl)carbamoyl) benzo[b]thio- phen-5- yl)difluoro- methyl)phos- phonic acid | 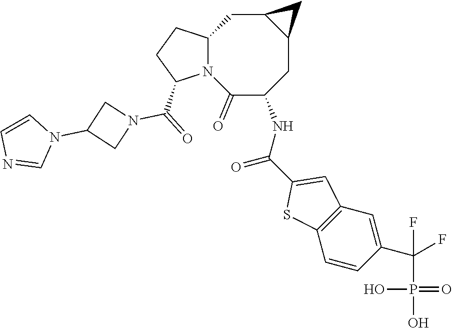 [SMILES: O=C(N[C@H]1C[C@@H]2C[C@@H]2C[C@H]2CC[C@@H](C(=O)N3CC(n4ccnc4)C3)N2C1=O)c1cc2cc(C(F)(F)P(=O)(O)O)ccc2s1] | 634.2 [M + H]⁺ | ¹H NMR (400 MHZ, CD₃OD) δ 9.26-8.77 (m, 1H), 8.34 (d, J = 14.0 Hz, 1H), 8.15- 7.98 (m, 2H), 7.94- 7.74 (m, 1H), 7.65- 7.39 (m, 2H), 5.49- 5.15 (m, 1H), 5.08- 4.90 (m, 1H), 4.85- 4.68 (m, 1H), 4.47- 4.33 (m, 2H), 4.24- 4.05 (m, 3H), 2.38- 2.30 (m, 1H), 2.21- 1.79 (m, 6H), 1.74- 1.56 (m, 1H), 1.32 (dd, J = 4.8, 7.4 Hz, 1H), 0.89-0.66 (m, 2H), 0.019-0.005 (m, 1H). |
| 95 | (difluoro(2- (((3S,6S,7aS, 8aR,9aR)-3- (3-fluoro-3- (morpholine- 4- carbonyl)aze- tidine-1- carbonyl)-5- oxodeca- hydro-1H- cyclopropa[d] pyrrolo[1,2- a]azocin-6- yl)carbamoyl) benzo[b]thio- phen-5- yl)methyl) phosphonic acid | 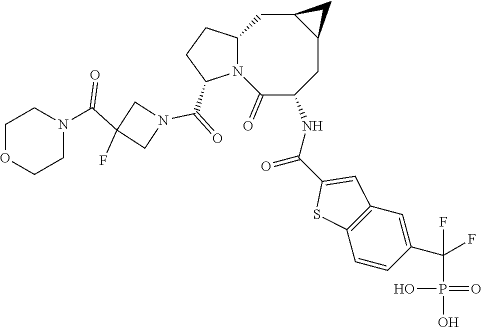 [SMILES: O=C(N[C@H]1C[C@@H]2C[C@@H]2C[C@H]2CC[C@@H](C(=O)N3CC(F)(C(=O)N4CCOCC4)C3)N2C1=O)c1cc2cc(C(F)(F)P(=O)(O)O)ccc2s1] | 699.2 [M + H]⁺ | ¹H NMR (400 MHZ, CD₃OD) δ 8.22-8.07 (m, 2H), 7.96-7.89 (m, 1H), 7.74 (d, J = 8.4 Hz, 1H), 5.07-4.98 (m, 1H), 4.90 (s, 1H), 4.58-4.48 (m, 2H), 4.29 (m, 1H), 3.73- 3.57 (m, 6H), 3.51- 3.36 (m, 3H), 2.52- 2.37 (m, 1H), 2.35- 2.26 (m, 2H), 2.26- 2.07 (m, 3H), 2.02- 1.89 (m, 2H), 1.81- 1.66 (m, 1H), 1.44- 1.32 (m, 1H), 0.99- 0.88 (m, 1H), 0.87- 0.77 (m, 1H), 0.11- 0.01 (m, 1H). |
| 96 | (difluoro(2- (((3S,6S,7aS, 8aR,9aR)-5- ox0-3-(3- (tetrahydro- 2H-pyran-4- yl)azetidine- 1- carbonyl)deca- hydro-1H- cyclopropa[d] pyrrolo[1,2- a]azocin-6- yl)carbamoyl) benzo[b]thio- phen-5- yl)methyl) phosphonic acid | 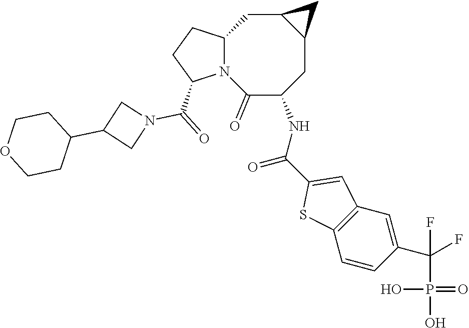 [SMILES: O=C(N[C@H]1C[C@@H]2C[C@@H]2C[C@H]2CC[C@@H](C(=O)N3CC(C4CCOCC4)C3)N2C1=O)c1cc2cc(C(F)(F)P(=O)(O)O)ccc2s1] | 651.7 [M + H]⁺ | ¹H NMR (400 MHZ, CD₃OD) δ 8.19 (s, 1H), 8.14 (d, J = 1.6 Hz, 1H), 7.95-7.90 (m, 1H), 7.74 (d, J = 8.6 Hz, 1H), 5.07-4.99 (m, 1H), 4.55-4.46 (m, 1H), 4.34-4.23 (m, 2H), 4.09-3.87 (m, 4H), 3.80-3.59 (m, 1H), 3.45-3.36 (m, 2H), 2.52-2.35 (m, 2H), 2.33-2.06 (m, 4H), 2.04-1.87 (m, 2H), 1.85-1.56 (m, 4H), 1.49-1.35 (m, 1H), 1.27-1.10 (m, 2H), 0.99-0.90 (m, 1H), 0.88-0.79 (m, 1H), 0.06 (q, J = 5.2 Hz, 1H). |
| 97 | (difluoro(2- (((3S,6S,7aS, 8aR,9aR)-5- ox0-3-(3- (pyridin-2- yl)azetidine- 1- carbonyl)deca- hydro-1H- cyclopropa[d] pyrrolo[1,2- a]azocin-6- yl)carbamoyl )benzo[b]thio- phen-5- yl)methyl) phosphonic acid | 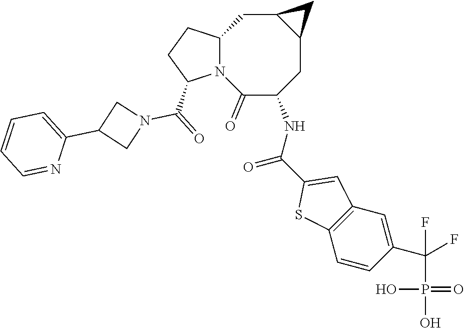 [SMILES: O=C(N[C@H]1C[C@@H]2C[C@@H]2C[C@H]2CC[C@@H](C(=O)N3CC(c4ccccn4)C3)N2C1=O)c1cc2cc(C(F)(F)P(=O)(O)O)ccc2s1] | 645.2 [M + H]⁺ | ¹H NMR (400 MHZ, CD₃OD) δ 8.61-8.47 (m, 1H), 8.21-8.07 (m, 2H), 7.95-7.79 (m, 2H), 7.77-7.70 (m, 1H), 7.64-7.38 (m, 1H), 7.38-7.30 (m, 1H), 5.07-4.97 (m, 1H), 4.56 (dd, J = 2.8, 8.8 Hz, 1H), 4.47-4.26 (m, 3H), 4.20 (dd, J = 6.4, 10.0 Hz, 1H), 4.15- 4.01 (m, 2H), 2.55- 2.40 (m, 1H), 2.36- 2.26 (m, 2H), 2.25- 2.12 (m, 2H), 2.09- 1.92 (m, 2H), 1.81- 1.66 (m, 1H), 1.52- 1.37 (m, 1H), 1.02- 0.92 (m, 1H), 0.89- 0.79 (m, 1H), 0.11- 0.01 (m, 1H). |
| 98 | (difluoro(2- (((3S,6S,7aS, 8aR,9aR)-3- ((R)-3- (morpholine- 4- carbonyl)py- rrolidine-1- carbonyl)-5- oxodeca- hydro-1H- cyclopropa[d] pyrrolo[1,2- a]azocin-6- yl)carbamoyl) benzo[b]thio- phen-5- yl)methyl) phosphonic acid | 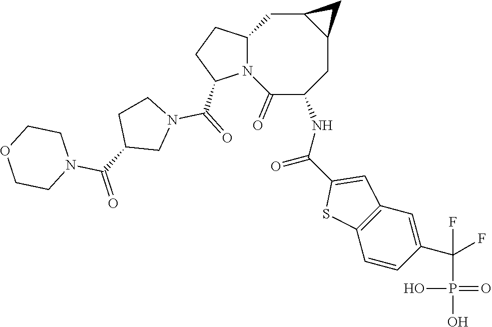 [SMILES: O=C(N[C@H]1C[C@@H]2C[C@@H]2C[C@H]2CC[C@@H](C(=O)N3CC[C@@H](C(=O)N4CCOCC4)C3)N2C1=O)c1cc2cc(C(F)(F)P(=O)(O)O)ccc2s1] | 695.3 [M + H]⁺ | ¹H NMR (400 MHZ, CD₃OD) δ 8.19 (s, 1H), 8.12 (s, 1H), 7.93 (d, J = 8.8 Hz, 1H), 7.74 (d, J = 8.8 Hz, 1H), 5.06- 5.01 (m, 1H), 4.74- 4.70 (m, 1H), 4.34- 4.25 (m, 1H), 3.80 (s, 1H), 3.70-3.54 (m, 11H), 3.49-3.41 (m, 1H), 2.52-2.42 (m, 1H), 2.36-2.30 (m, 1H), 2.28-2.12 (m, 5H), 2.02-1.94 (m, 2H), 1.81-1.69 (m, 1H), 1.55-1.42 (m, 1H), 1.04-0.93 (m, 1H), 0.89-0.82 (m, 1H), 0.10-0.03 (m, 1H). |
| 99 | (difluoro(2- (((3S,6S,7aS, 8aR,9aR)-3- ((S)-3- (morpholine- 4- carbonyl)py- rrolidine-1- carbonyl)-5- oxodeca- hydro-1H- cyclopropa[d] pyrrolo[1,2- a]azocin-6- yl)carbamoyl) benzo[b]thio- phen-5- yl)methyl) phosphonic acid | 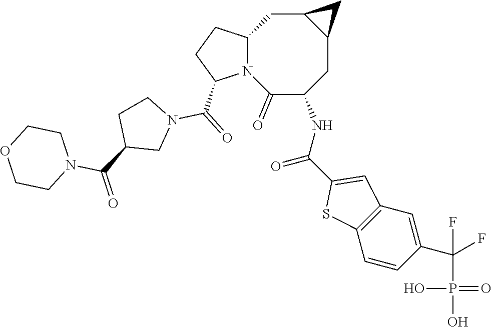 [SMILES: O=C(N[C@H]1C[C@@H]2C[C@@H]2C[C@H]2CC[C@@H](C(=O)N3CC[C@H](C(=O)N4CCOCC4)C3)N2C1=O)c1cc2cc(C(F)(F)P(=O)(O)O)ccc2s1] | 695.2 [M + H]⁺ | ¹H NMR (400 MHZ, CD₃OD) δ 8.19 (s, 1H), 8.12 (s, 1H), 7.92 (d, J = 8.4 Hz, 1H), 7.75 (d, J = 8.4 Hz, 1H), 5.11-4.99 (m, 1H), 4.78-4.70 (m, 1H), 4.29 (s, 1H), 4.15- 3.95 (m, 1H), 3.75- 3.45 (m, 10H), 3.19- 2.64 (m, 1H), 2.50- 2.46 (m, 1H), 2.35- 2.11 (m, 6H), 2.10- 1.91 (m, 3H), 1.83- 1.65 (m, 1H), 1.50- 1.42 (m, 1H), 1.05- 0.91 (m, 1H), 0.91- 0.80 (m, 1H), 0.08- 0.05 (m, 1H). |
| 100 | (difluoro(2- (((3S,6S,7aS, 8aR,9aR)-5- ox0-3-(3- (pyridin-3- yl)azetidine- 1- carbonyl)deca- hydro-1H- cyclopropa[d] pyrrolo[1,2- a]azocin-6- yl)carbamoyl) benzo[b]thio- phen-5- yl)methyl) phosphonic acid | 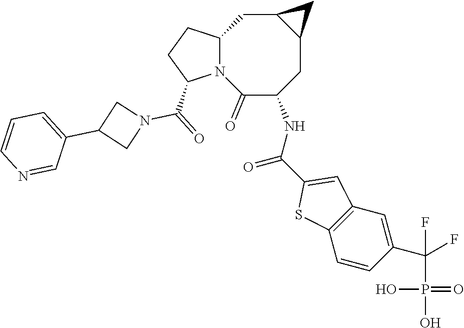 [SMILES: O=C(N[C@H]1C[C@@H]2C[C@@H]2C[C@H]2CC[C@@H](C(=O)N3CC(c4cccnc4)C3)N2C1=O)c1cc2cc(C(F)(F)P(=O)(O)O)ccc2s1] | 645.2 [M + H]⁺ | ¹H NMR (400 MHZ, CD₃OD) δ 8.54 (d, J = 2.0 Hz, 1H), 8.48-8.37 (m, 1H), 8.22-8.10 (m, 2H), 7.92 (d, J = 10.4 Hz, 2H), 7.80-7.69 (m, 1H), 7.53-7.38 (m, 1H), 5.05 (s, 2H), 4.70 (d, J = 6.8 Hz, 1H), 4.60-4.53 (m, 1H), 4.50-4.25 (m, 2H), 4.07-3.89 (m, 2H), 2.54-2.42 (m, 1H), 2.36-2.10 (m, 4H), 2.06-1.91 (m, 2H), 1.86-1.69 (m, 1H), 1.53-1.31 (m, 1H), 1.05-0.75 (m, 2H), 0.07 (s, 1H). |
| 101 | ((2- (((3S,6S,7aS, 8aR,9aR)-3- ((3-(1- amino-1- oxopropan-2- yl)-[1,1'- biphenyl]-4- yl)carb- amoyl)-5- oxodecahydr o-1H- cyclopropa[d pyrrolo[1,2- a]azocin-6- yl)carbamoyl) benzo[b]thio- phen-5- yl)difluoro- methyl) phosphonic acid | 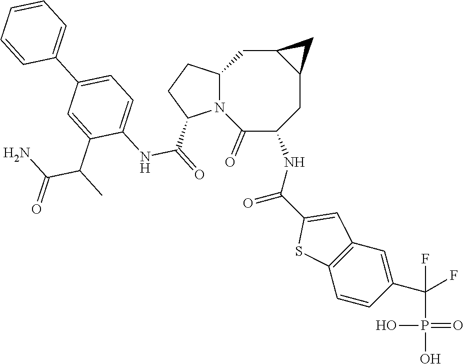 [SMILES: CC(C(N)=O)c1cc(-c2ccccc2)ccc1NC(=O)[C@@H]1CC[C@@H]2C[C@H]3C[C@H]3C[C@H](NC(=O)c3cc4cc(C(F)(F)P(=O)(O)O)ccc4s3)C(=O)N21] | 751.3 [M + H]⁺ | ¹H NMR (400 MHZ, CD₃OD) δ 8.20 (s, 1H), 8.14-8.09 (m, 1H), 7.93-7.88 (m, 1H), 7.76 (d, J = 8.4 Hz, 1H), 5.07-4.97 (m, 1H), 4.74-4.63 (m, 1H), 4.52-4.38 (m, 1H), 4.34-4.11 (m, 5H), 4.06-3.92 (m, 2H), 3.85-3.77 (m, 1H), 3.57-3.47 (m, 1H), 3.30-3.24 (m, 3H), 3.18-2.91 (m, 1H), 2.51-2.39 (m, 1H), 2.36-2.06 (m, 4H), 2.02-1.88 (m, 2H), 1.81-1.66 (m, 1H), 1.48-1.34 (m, 1H), 1.03-0.78 (m, 2H), 0.10-0.01 (m, 1H). |
| 102 | (difluoro(2- (((3S,6S,7aS, 8aR,9aR)-3- (3-(3- methoxy- azetidine-1- carbonyl)aze- tidine-1- carbonyl)-5- oxodeca- hydro-1H- cyclopropa[d] pyrrolo[1,2- a]azocin-6- yl)carbamoyl) benzo[b]thio- phen-5- yl)methyl) phosphonic acid | 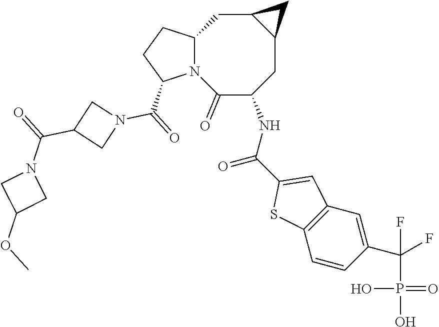 [SMILES: COC1CN(C(=O)C2CN(C(=O)[C@@H]3CC[C@@H]4C[C@H]5C[C@H]5C[C@H](NC(=O)c5cc6cc(C(F)(F)P(=O)(O)O)ccc6s5)C(=O)N43)C2)C1] | 681.2 [M + H]⁺ | ¹H NMR (400 MHZ, CD₃OD) δ 8.20 (s, 1H), 8.14-8.09 (m, 1H), 7.93-7.88 (m, 1H), 7.76 (d, J = 8.4 Hz, 1H), 5.07-4.97 (m, 1H), 4.74-4.63 (m, 1H), 4.52-4.38 (m, 1H), 4.34-4.11 (m, 5H), 4.06-3.92 (m, 2H), 3.85-3.77 (m, 1H), 3.57-3.47 (m, 1H), 3.30-3.24 (m, 3H), 3.18-2.91 (m, 1H), 2.51-2.39 (m, 1H), 2.36-2.06 (m, 4H), 2.02-1.88 (m, 2H), 1.81-1.66 (m, 1H), 1.48-1.34 (m, 1H), 1.03-0.78 (m, 2H), 0.10-0.01 (m, 1H). |
| 103 | ((2- (((3S,6S,7aS, 8aR,9aR)-3- (3-(2H- tetrazol-5- yl)azetidine- 1-carbonyl)- 5- oxodeca- hydro-1H- cyclopropa[d] pyrrolo[1,2- a]azocin-6- yl)carbamoyl) benzo[b]thio- phen-5- yl)difluoro- methyl) phosphonic acid | 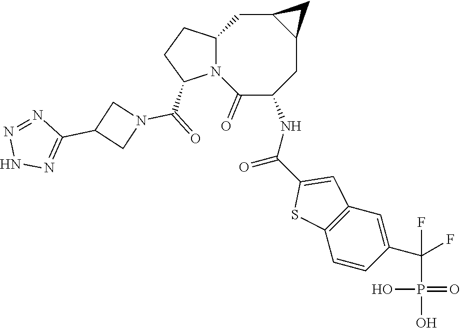 [SMILES: O=C(N[C@H]1C[C@@H]2C[C@@H]2C[C@H]2CC[C@@H](C(=O)N3CC(c4nn[nH]n4)C3)N2C1=O)c1cc2cc(C(F)(F)P(=O)(O)O)ccc2s1] | 636 [M + H]⁺ | ¹H NMR (400 MHZ, CD₃OD) δ 8.22-8.09 (m, 2H), 7.93 (d, J = 8.4 Hz, 1H), 7.74 (d, J = 8.4 Hz, 1H), 5.09- 4.92 (m, 2H), 4.76- 4.70 (m, 1H), 4.54- 4.46 (m, 2H), 4.35- 4.25 (m, 2H), 4.25- 4.05 (m, 1H), 2.54- 2.39 (m, 1H), 2.34- 2.10 (m, 4H), 2.04- 1.92 (m, 2H), 1.80- 1.68 (m, 1H), 1.49- 1.36 (m, 1H), 1.35- 1.27 (m, 1H), 1.03- 0.91 (m, 1H), 0.88- 0.79 (m, 1H), 0.13- 0.02 (m, 1H). |
| 104 | ((2- (((3S,6S,7aS, 8aR,9aR)-3- (4-(1H- pyrazole-4- carbonyl)pip- erazine-1- carbonyl)-5- oxode- cahydro-1H- cyclopropa[d] pyrrolo[1,2- a]azocin-6- yl)carbamoyl) benzo[b]thio- phen-5- yl)difluoro- methyl)phos- phonic acid | 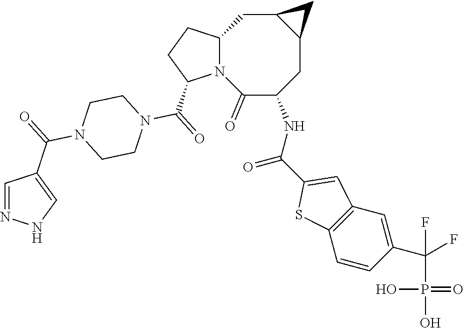 [SMILES: O=C(N[C@H]1C[C@@H]2C[C@@H]2C[C@H]2CC[C@@H](C(=O)N3CCN(C(=O)c4cn[nH]c4)CC3)N2C1=O)c1cc2cc(C(F)(F)P(=O)(O)O)ccc2s1] | 691.2 [M + H]⁺ | ¹H NMR (400 MHZ, CD₃OD) δ 8.20-8.08 (m, 2H), 7.97-7.87 (m, 3H), 7.73 (d, J = 8.8 Hz, 1H), 5.13-4.95 (m, 2H), 4.36-4.26 (m, 1H), 3.97-3.74 (m, 5H), 3.72-3.42 (m, 3H), 2.53-2.30 (m, 2H), 2.27-2.14 (m, 3H), 2.06-1.90 (m, 2H), 1.83-1.69 (m, 1H), 1.5o-1.38 (m, 1H), 1.09-0.96 (m, 1H), 0.87 (m, 1H), 0.08 (m, 1H). |
| 105 | ((2- (((3S,6S,7aS, 8aR,9aR)-3- (4-(1H- pyrazole-3- carbonyl)pip- erazine-1- carbonyl)-5- oxodeca- hydro-1H- cyclopropa[d] pyrrolo[1,2- a]azocin-6- yl)carbamoyl) benzo[b]thio- phen-5- yl)difluoro- methyl)phos- phonic acid | 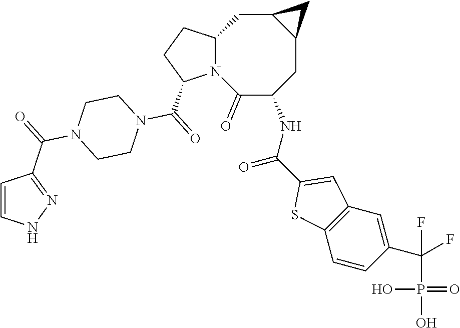 [SMILES: O=C(N[C@H]1C[C@@H]2C[C@@H]2C[C@H]2CC[C@@H](C(=O)N3CCN(C(=O)c4cc[nH]n4)CC3)N2C1=O)c1cc2cc(C(F)(F)P(=O)(O)O)ccc2s1] | 691.2 [M + H]⁺ | ¹H NMR (400 MHZ, CD₃OD) δ 8.21-8.08 (m, 2H), 7.93 (d, J = 8.8 Hz, 1H), 7.81-7.66 (m, 2H), 6.66 (d, J = 2.2 Hz, 1H), 5.11-4.95 (m, 2H), 4.30 (d, J = 6.4 Hz, 1H), 4.16-3.97 (m, 2H), 3.95-3.61 (m, 5H), 3.52 (s, 1H), 2.61- 2.41 (m, 1H), 2.38- 2.28 (m, 1H), 2.20 (d, J = 6.0 Hz, 2H), 2.07- 1.88 (m, 2H), 1.86- 1.68 (m, 1H), 1.52- 1.37 (m, 1H), 1.02 (m, 1H), 0.87 (m, 1H), 0.07 (q, J = 5.2 Hz, 1H). |
| 106 | (difluoro(2- (((3S,6S,7aR, 8aS,9aR)-5- ox0-3-(3- (pyridin-2- yl)azetidine- 1- carbonyl)de- cahydro-1H- cyclopropa[d] pyrrolo[1,2- a]azocin-6- yl)carbamoyl )benzo[b]thio- phen-5- yl)methyl) phosphonic acid | 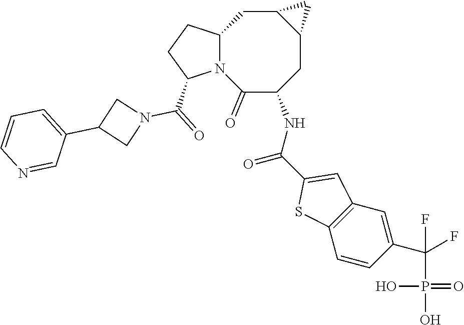 [SMILES: O=C(N[C@H]1C[C@H]2C[C@H]2C[C@H]2CC[C@@H](C(=O)N3CC(c4cccnc4)C3)N2C1=O)c1cc2cc(C(F)(F)P(=O)(O)O)ccc2s1] | 645.2 [M + H]⁺ | ¹H NMR (400 MHZ, CD₃OD) δ 8.60-8.42 (m, 1H), 8.23-8.06 (m, 2H), 7.92 (dd, J = 8.8, 16.0 Hz, 1H), 7.81- 7.71 (m, 2H), 7.55- 7.34 (m, 1H), 7.34- 7.23 (m, 1H), 4.94 (s, 1H), 4.68 (m, 1H), 4.53- 4.39 (m, 2H), 4.38- 4.20 (m, 2H), 4.16- 4.00 (m, 2H), 2.42- 2.10 (m, 4H), 2.08- 1.84 (m, 3H), 1.79- 1.63 (m, 1H), 1.52- 1.37 (m, 1H), 1.35- 1.21 (m, 1H), 0.91- 0.79 (m, 1H), 0.19 (m, 1H). |
| 107 | ((2- (((3S,6S,7aR, 8aS,9aR)-3- (3-(1H- imidazol-1- yl)azetidine- 1-carbonyl)- 5- oxodeca- hydro-1H- cyclopropa[d] pyrrolo[1,2- a]azocin-6- yl)carbamoyl) benzo[b]thio- phen-5- yl)difluoro- methyl)phos- phonic acid | 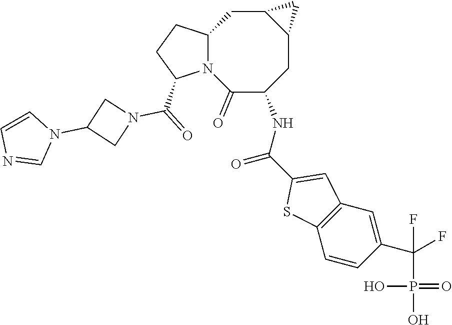 [SMILES: O=C(N[C@H]1C[C@H]2C[C@H]2C[C@H]2CC[C@@H](C(=O)N3CC(n4ccnc4)C3)N2C1=O)c1cc2cc(C(F)(F)P(=O)(O)O)ccc2s1] | 634 [M + H]⁺ | ¹H NMR (400 MHZ, CD₃OD) δ 8.62 (s, 1H), 8.42 (s, 1H), 8.30- 8.17 (m, 1H), 8.13- 8.05 (m, 1H), 7.94 (d, J = 8.8 Hz, 2H), 7.82- 7.63 (m, 1H), 7.29 (s, 1H), 5.42-5.24 (m, 1H), 4.91 (d, J = 5.8 Hz, 2H), 4.63-4.27 (m, 4H), 4.19 (dd, J = 5.2, 11.2 Hz, 1H), 2.30- 2.11 (m, 3H), 2.04- 1.83 (m, 3H), 1.74- 1.61 (m, 1H), 1.47 (dd, J = 4.8, 7.6 Hz, 1H), 1.30 (d, J = 7.6 Hz, 1H), 0.87 (dd, J = 3.2, 8.0 Hz, 1H), 0.18 (q, J = 4.8 Hz, 1H). |
| 108 | ((2- (((3S,6S,7aS, 8aR,9aR)-3- (3-(1,1- dioxidothio- morpholino) azetidine-1- carbonyl)-5- oxodeca- hydro-1H- cyclopropa[d] pyrrolo[1,2- a]azocin-6- yl)carbamoyl )benzo[b]thio- phen-5- yl)difluoro- methyl)phos- phonic acid | 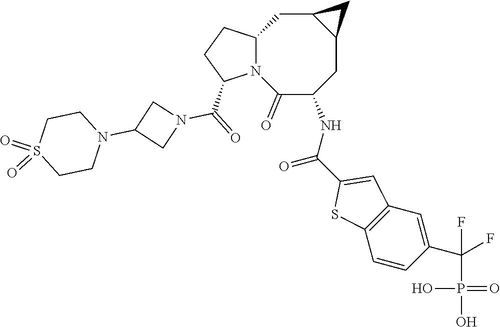 [SMILES: O=C(N[C@H]1C[C@@H]2C[C@@H]2C[C@H]2CC[C@@H](C(=O)N3CC(N4CCS(=O)(=O)CC4)C3)N2C1=O)c1cc2cc(C(F)(F)P(=O)(O)O)ccc2s1] | 701.1 [M + H]⁺ | ¹H NMR (400 MHZ, CD₃OD) δ 8.24-8.10 (m, 2H), 7.91 (d, J = 8.4 Hz, 1H), 7.76 (d, J = 8.4 Hz, 1H), 5.02 (t, J = 9.2 Hz, 1H), 4.66- 4.39 (m, 2H), 4.35- 4.25 (m, 2H), 4.15- 3.92 (m, 2H), 3.89- 3.74 (m, 1H), 3.49- 3.43 (m, 1H), 3.20- 3.04 (m, 4H), 2.89 (s, 4H), 2.52-2.40 (m, 1H), 2.35-2.06 (m, 4H), 2.02-1.89 (m, 2H), 1.8o-1.67 (m, 1H), 1.49-1.33 (m, 1H), 1.00-0.78 (m, 2H), 0.08-0.04 (m, 1H). |
| 109 | (difluoro(2- (((3S,6S,7aS, 8aR,9aR)-3- (3-((R)-3- hydroxy- pyrrolidine-1- carbonyl) azetidine-1- carbonyl)-5- oxodeca- hydro-1H- cyclopropa[d] pyrrolo[1,2- a]azocin-6- yl)carbamoyl) benzo[b]thio- phen-5- yl)methyl) phosphonic acid | 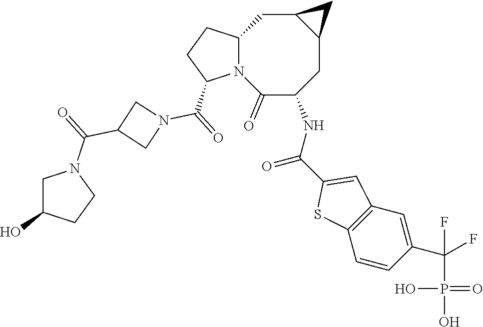 [SMILES: O=C(N[C@H]1C[C@@H]2C[C@@H]2C[C@H]2CC[C@@H](C(=O)N3CC(C(=O)N4CC[C@@H](O)C4)C3)N2C1=O)c1cc2cc(C(F)(F)P(=O)(O)O)ccc2s1] | 681.3 [M + H]⁺ | ¹H NMR (400 MHZ, CD₃OD) δ 8.25-8.04 (m, 2H), 7.96-7.83 (m, 1H), 7.75 (d, J = 8.4 Hz, 1H), 5.11-4.99 (m, 1H), 4.78-4.65 (m, 1H), 4.55-4.47 (m, 1H), 4.44-4.36 (m, 1H), 4.43-4.17 (m, 2H), 4.10-4.01 (m, 1H), 3.79-3.61 (m, 1H), 3.59-3.43 (m, 4H), 3.31-3.21 (m, 1H) 2.53-2.39 (m, 1H), 2.35-2.07 (m, 4H), 2.05-1.83 (m, 4H), 1.79-1.65 (m, 1H), 1.48-1.32 (m, 1H), 1.01-0.89 (m, 1H), 0.88-0.82 (m, 1H), 0.12-0.02 (m, 1H). |
| 110 | (difluoro(2- (((3S,6S,7aS, 8aR,9aR)-3- (3-(4- hydroxy-4- methylpip- eridin-1- yl)azetidine- 1-carbonyl)- 5- oxodeca- hydro-1H- cyclopropa[d] pyrrolo[1,2- a]azocin-6- yl)carbamoyl) benzo[b]thio- phen-5- yl)methyl) phosphonic acid | 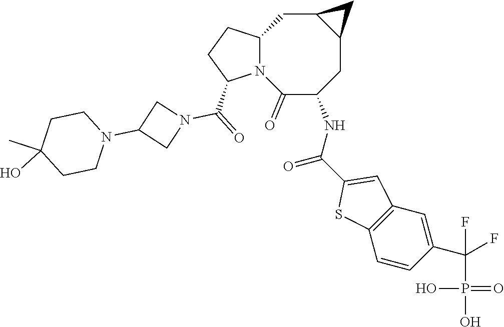 [SMILES: CC1(O)CCN(C2CN(C(=O)[C@@H]3CC[C@@H]4C[C@H]5C[C@H]5C[C@H](NC(=O)c5cc6cc(C(F)(F)P(=O)(O)O)ccc6s5)C(=O)N43)C2)CC1] | 681.3 [M + H]⁺ | ¹H NMR (400 MHZ, CD₃OD) δ 8.19-8.08 (m, 2H), 7.97-7.88 (m, 1H), 7.74 (d, J = 8.8 Hz, 1H), 5.01 (t, J = 8.8 Hz, 1H), 4.78-4.62 (m, 2H), 4.51-4.39 (m, 2H), 4.37-4.24 (m, 2H), 4.23-4.07 (m, 2H), 4.04-3.85 (m, 1H), 3.79-3.62 (m, 1H), 3.15-3.01 (m, 1H), 2.94 (s, 2H), 2.85- 2.73 (m, 1H), 2.52- 2.41 (m, 1H), 2.32- 2.07 (m, 4H), 2.03- 1.90 (m, 2H), 1.84- 1.66 (m, 5H), 1.45- 1.32 (m, 1H), 0.96- 0.79 (m, 2H), 0.07 (m, 1H). |
| 111 | ((2- (((3S,6S,7aS, 8aR,9aR)-3- (3-(1H- pyrazol-3- yl)azetidine- 1-carbonyl)- 5- oxodeca- hydro-1H- cyclopropa[d] pyrrolo[1,2- a]azocin-6- yl)carbamoyl) benzo[b]thio- phen-5- yl)difluoro- methyl)phos- phonic acid | 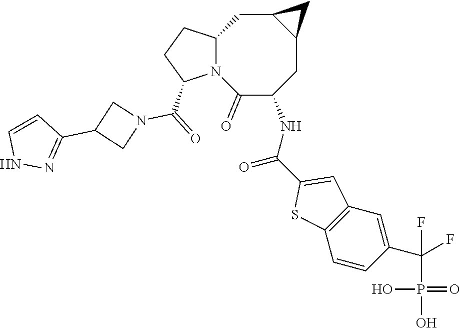 [SMILES: O=C(N[C@H]1C[C@@H]2C[C@@H]2C[C@H]2CC[C@@H](C(=O)N3CC(c4cc[nH]n4)C3)N2C1=O)c1cc2cc(C(F)(F)P(=O)(O)O)ccc2s1] | 653.2 [M + H]⁺ | ¹H NMR (400 MHZ, CD₃OD) δ 8.24-8.09 (m, 2H), 8.00-7.87 (m, 1H), 7.73 (d, J = 8.4 Hz, 1H), 7.61-7.55 (m, 1H), 6.54-6.28 (m, 1H), 5.09-5.00 (m, 1H), 4.70-4.62 (m, 1H), 4.58-4.50 (m, 1H), 4.47-4.37 (m, 1H), 4.35-4.24 (m, 2H), 4.10-3.89 (m, 2H), 2.55-2.40 (m, 1H), 2.36-2.09 (m, 4H), 2.05-1.91 (m, 2H), 1.81-1.67 (m, 1H), 1.53-1.34 (m, 1H), 1.04-0.91 (m, 1H), 0.90-0.78 (m, 1H), 0.09-0.01 (m, 1H). |
| 112 | (difluoro(2- (((3S,6S,7aS, 8aR,9aR)-5- oxo-3-(((R)- 1-(pyridin-3- yl)pyrrolidin- 3- yl)carbamoyl) decahydro- 1H- cyclopropa[d] pyrrolo[1,2- a]azocin-6- yl)carbamoyl) benzo[b]thio- phen-5- yl)methyl) phosphonic acid | 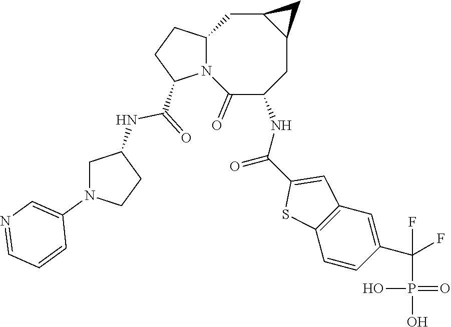 [SMILES: O=C(N[C@H]1C[C@@H]2C[C@@H]2C[C@H]2CC[C@@H](C(=O)N[C@@H]3CCN(c4cccnc4)C3)N2C1=O)c1cc2cc(C(F)(F)P(=O)(O)O)ccc2s1] | 674.3 [M + H]⁺ | ¹H NMR (400 MHZ, CD₃OD) δ 8.89 (m, 1H), 8.22 (s, 1H), 8.09 (m,1H), 8.02 (s, 1H), 7.96 (m,1H), 7.86- 7.79 (m, 2H), 7.58 (m, 1H), 7.24 (m, 1H), 6.98 (m, 1H), 4.87 (d, J = 7.6 Hz, 1H), 4.51-4.34 (m, 2H), 4.12 (d, J = 6.8 Hz, 1H), 3.43 (dd, J = 6.0, 10.0 Hz, 2H), 3.35-3.28 (m, 2H), 3.02 (dd, J = 2.4, 10.0 Hz, 1H), 2.41-2.30 (m, 1H), 2.24-2.05 (m, 3H), 2.03-1.95 (m, 1H), 1.95-1.83 (m, 3H), 1.77 (m, 1H), 1.59 (d, J = 4.8 Hz, 1H), 1.51-1.42 (m, 1H), 1.26-1.18 (m, 1H), 1.02-0.84 (m, 1H), 0.70-0.56 (m, 1H), 0.43 (m, 1H),-0.08 (q, J = 4.8 Hz, 1H). |
| 113 | (difluoro(2- (((3S,6S,7aS, 8aR,9aR)-5- ox0-3-(4- (pyridin-2- yl)piperazine- 1- carbonyl)de- cahydro-1H- cyclopropa[d] pyrrolo[1,2- a]azocin-6- yl)carbamoyl) benzo[b]thio- phen-5- yl)methyl) phosphonic acid | 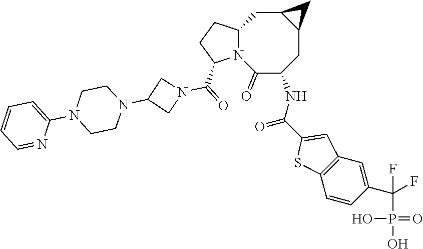 [SMILES: O=C(N[C@H]1C[C@@H]2C[C@@H]2C[C@H]2CC[C@@H](C(=O)N3CC(N4CCN(c5ccccn5)CC4)C3)N2C1=O)c1cc2cc(C(F)(F)P(=O)(O)O)ccc2s1] | 674.1 [M + H]⁺ | ¹H NMR (400 MHZ, CD₃OD) δ 8.15-8.07 (m, 2H), 8.00-7.93 (m, 1H), 7.85 (d, J = 8.0 Hz, 1H), 7.73-7.61 (m, 2H), 6.90 (d, J = 8.0 Hz, 1H), 6.73 (m, 1H), 5.04-5.01 (m, 1H), 4.37-4.27 (m, 1H), 4.02-3.90 (m, 1H), 3.89-3.81 (m, 1H), 3.78-3.70 (m, 2H), 3.70-3.57 (m, 4H), 3.56-3.47 (m, 2H), 2.46 (m, 1H), 2.33 (m, 1H), 2.24-2.19 (m, 2H), 2.01-1.90 (m, 2H), 1.76 (m, 1H), 1.50- 1.40 (m, 1H), 1.08- 0.96 (m, 1H), 0.87 (m, 1H), 0.12-0.03 (m, 1H). |
| 114 | (difluoro(2- (((3S,6S,7aS, 8aR,9aR)-3- (3-((R)-3- methoxypy- rrolidine-1- carbonyl)aze- tidine-1- carbonyl)-5- oxodeca- hydro-1H- cyclopropa[d] pyrrolo[1,2- a]azocin-6- yl)carbamoyl) benzo[b]thio- phen-5- yl)methyl) phosphonic acid | 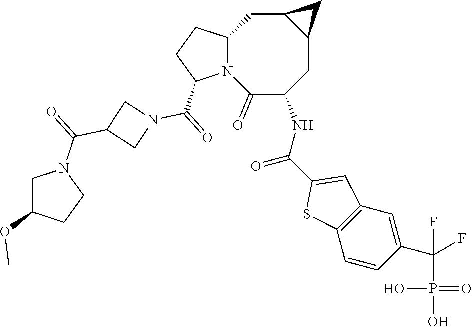 [SMILES: CO[C@@H]1CCN(C(=O)C2CN(C(=O)[C@@H]3CC[C@@H]4C[C@H]5C[C@H]5C[C@H](NC(=O)c5cc6cc(C(F)(F)P(=O)(O)O)ccc6s5)C(=O)N43)C2)C1] | 695.2 [M + H]⁺ | ¹H NMR (400 MHZ, CD₃OD) δ 8.28-8.03 (m, 2H), 8.00-7.86 (m, 1H), 7.74 (d, J = 8.4 Hz, 1H), 5.11-4.98 (m, 1H), 4.79-4.67 (m, 1H), 4.56-4.44 (m, 1H), 4.42-4.34 (m, 1H), 4.33-4.15 (m, 2H), 4.12-3.96 (m, 1H), 3.78-3.65 (m, 1H), 3.64-3.36 (m, 3H), 3.33-3.32 (m, 3H), 3.29-3.27 (m, 1H), 3.20-2.68 (m, 1H), 2.49-2.45 (m, 1H), 2.36-1.87 (m, 8H), 1.8o-1.65 (m, 1H), 1.51-1.25 (m, 1H), 0.99-0.78 (m, 2H), 0.13-0.00 (m, 1H). |
| 115 | (difluoro(2- (((3S,6S,7aS, 8aR,9aR)-5- ox0-3-(4- (tetrahydro- 2H-pyran-4- yl)piperazine- 1- carbonyl) decahydro-1H- cyclopropa[d] pyrrolo[1,2- a]azocin-6- yl)carbamoyl) benzo[b]thio- phen-5- yl)methyl) phosphonic acid | 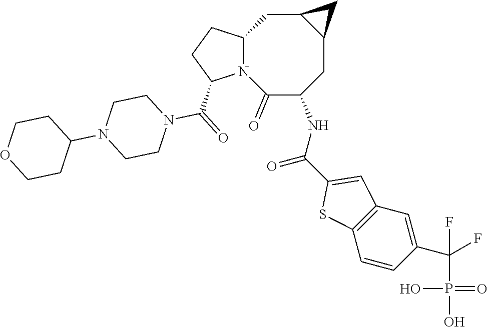 [SMILES: O=C(N[C@H]1C[C@@H]2C[C@@H]2C[C@H]2CC[C@@H](C(=O)N3CCN(C4CCOCC4)CC3)N2C1=O)c1cc2cc(C(F)(F)P(=O)(O)O)ccc2s1] | 681.2 [M + H]⁺ | ¹H NMR (400 MHZ, CD₃OD) δ 8.10 (s, 1H), 8.08-8.00 (m, 1H), 7.87 (d, J = 8.4 Hz, 1H), 7.71-7.60 (m, 1H), 5.01-4.97 (m, 1H), 4.23 (d, J = 4.0 Hz, 1H), 3.89 (m, 2H), 3.79 (s, 2H), 3.55 (d, J = 7.0 Hz, 1H), 3.45- 3.36 (m, 1H), 3.35- 3.27 (m, 2H), 2.82 (s, 3H), 2.71 (d, J = 12.0 Hz, 2H), 2.47-2.34 (m, 1H), 2.29-2.19 (m, 1H), 2.11 (s, 3H), 1.98- 1.85 (m, 2H), 1.80 (d, J = 8.0 Hz, 3H), 1.71- 1.62 (m, 1H), 1.58- 1.46 (m, 2H), 1.36 (s, 1H), 1.00-0.88 (m, 1H), 0.86-0.76 (m, 1H), 0.06-- 0.05 (m, 1H). |
| 116 | ((2- (((3S,6S,7aS, 9aR)-3-(((R)- 1- acetylpy- rrolidin-3- yl)carba- moyl)-5- oxodeca- hydro-1H- cyclopropa[d] pyrrolo[1,2- a]azocin-6- yl)carbamoyl) benzo[b]thio- phen-5- yl)difluoro- methyl)phos- phonic acid | 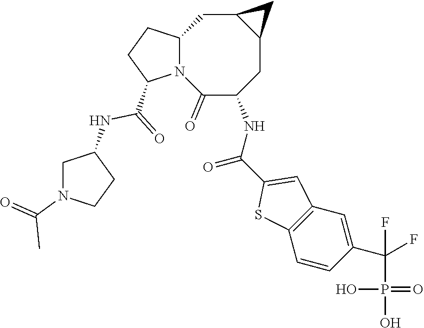 [SMILES: CC(=O)N1CC[C@@H](NC(=O)[C@@H]2CC[C@@H]3C[C@H]4C[C@H]4C[C@H](NC(=O)c4cc5cc(C(F)(F)P(=O)(O)O)ccc5s4)C(=O)N32)C1] | 639 [M + H]⁺ | ¹H NMR (400 MHZ, CD₃OD) δ 8.22-8.15 (m, 1H), 8.13 (s, 1H), 7.93 (d, J = 8.4 Hz, 1H), 7.74 (d, J = 8.4 Hz, 1H), 5.03 (t, J = 9.2 Hz, 1H), 4.64-4.51 (m, 1H), 4.39-4.21 (m, 2H), 3.77-3.46 (m, 2H), 3.41-3.33 (m, 2H), 2.53-2.40 (m, 1H), 2.32-2.09 (m, 5H), 2.06 (s, 2H), 2.01 (s, 2H), 2.0o-1.84 (m, 2H), 1.78-1.64 (m, 1H), 1.23-0.98 (m, 1H), 0.95-0.79 (m, 2H), 0.15-0.06 (m, 1H). |
| 117 | ((2- (((3S,6S,7aS, 8aR,9aR)-3- (((S)-1- acetylpy- rrolidin-3- yl)carba- moyl)-5- oxodeca- hydro-1H- cyclopropa[d] pyrrolo[1,2- a]azocin-6- yl)carbamoyl )benzo[b]thio- phen-5- yl)difluoro- methyl)phos- phonic acid | 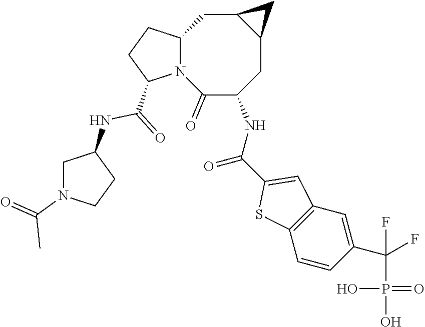 [SMILES: CC(=O)N1CC[C@H](NC(=O)[C@@H]2CC[C@@H]3C[C@H]4C[C@H]4C[C@H](NC(=O)c4cc5cc(C(F)(F)P(=O)(O)O)ccc5s4)C(=O)N32)C1] | 639.1 [M + H]⁺ | ¹H NMR (400 MHZ, CD₃OD) 88.19 (s, 1H), 8.13 (s, 1H), 7.93 (d, J = 8.4 Hz, 1H), 7.74 (d, J = 8.8 Hz, 1H), 5.06-5.01 (m, 1H), 4.57-4.50 (m, 1H), 4.38-4.24 (m, 2H), 3.68-3.54 (m, 2H), 3.54-3.45 (m, 1H), 3.40-3.34 (m, 1H), 2.52-2.42 (m, 1H), 2.31-2.26 (m, 1H), 2.24-2.11 (m, 3H), 2.1o-1.98 (m, 6H), 1.96-1.82 (m, 1H), 1.77 1.68 (m, 1H), 1.28-1.12 (m, 1H), 0.98-0.83 (m, 2H), 0.15-0.05 (m, 1H). |
| 118 | ((2- (((3S,6S,7aS, 8aR,9aR)-3- (((R)-1- carbamoyl- pyrrolidin-3- yl)carba- moyl)-5- oxodeca- hydro-1H- cyclopropa[d] pyrrolo[1,2- a]azocin-6- yl)carbamoyl) benzo[b]thio- phen-5- yl)difluoro- methyl)phos- phonic acid | 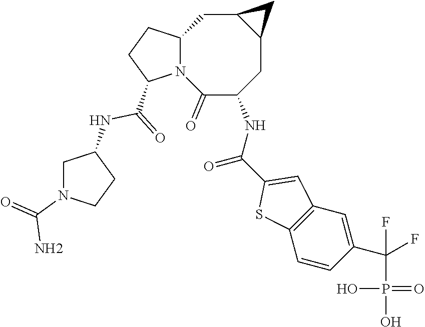 [SMILES: NC(=O)N1CC[C@@H](NC(=O)[C@@H]2CC[C@@H]3C[C@H]4C[C@H]4C[C@H](NC(=O)c4cc5cc(C(F)(F)P(=O)(O)O)ccc5s4)C(=O)N32)C1] | 639.9 [M + H]⁺ | ¹H NMR (400 MHZ, CD₃OD) δ 8.18 (s, 1H), 8.13 (s, 1H), 7.92 (d, J = 8.4 Hz, 1H), 7.74 (d, J = 8.4 Hz, 1H), 5.03 (t, J = 9.2 Hz, 1H), 4.61 (d, J = 7.6 Hz, 1H), 4.36-4.22 (m, 2H), 3.52 (dd, J = 5.6, 10.8 Hz, 1H), 3.47- 3.38 (m, 2H), 3.18 (d, J = 9.6 Hz, 1H), 2.53- 2.42 (m, 1H), 2.30- 2.12 (m, 4H), 2.10- 1.88 (m, 4H), 1.70 (m, 1H), 1.08-0.96 (m, 1H), 0.94-0.84 (m, 2H), 0.08 (d, J = 5.2 Hz, 1H). |
| 119 | (difluoro(2- (((3S,6S,7aS, 8aR,9aR)-5- oxo-3-(((R)- 1- (tetrahydro- 2H-pyran-4- yl)pyrrolidin- 3- yl)carbamoyl) decahydro- 1H- cyclopropa[d] pyrrolo[1,2- a]azocin-6- yl)carbamoyl) benzo[b]thio- phen-5- yl)methyl) phosphonic acid | 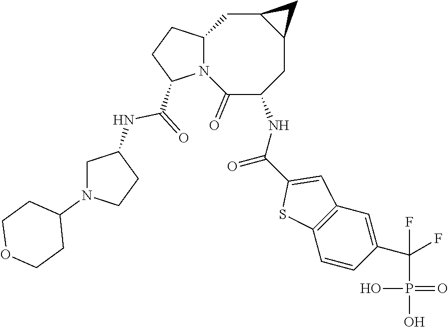 [SMILES: O=C(N[C@H]1C[C@@H]2C[C@@H]2C[C@H]2CC[C@@H](C(=O)N[C@@H]3CCN(C4CCOCC4)C3)N2C1=O)c1cc2cc(C(F)(F)P(=O)(O)O)ccc2s1] | 681 [M + H]⁺ | ¹H NMR (400 MHZ, CD₃OD) δ 8.22-8.12 (m, 2H), 7.93 (d, J = 8.4 Hz, 1H), 7.74 (d, J = 8.4 Hz, 1H), 5.04 (t, J = 8.8 Hz, 1H), 4.47 (d, J = 4.4 Hz, 1H), 4.41- 4.32 (m, 1H), 4.28 (d, J = 6.8 Hz, 1H), 3.96- 3.85 (m, 2H), 3.50- 3.33 (m, 2H), 3.29- 3.25 (m, 2H), 3.15- 3.00 (m, 3H), 2.53- 2.43 (m, 1H), 2.37- 2.25 (m, 2H), 2.21- 2.08 (m, 3H), 2.04- 1.91 (m, 3H), 1.83 (t, J = 15.2 Hz, 2H), 1.75- 1.67 (m, 1H), 1.65- 1.49 (m, 2H), 1.33- 1.25 (m, 1H), 1.03- 0.93 (m, 1H), 0.86 (m, 1H), 0.08 (m, 1H). |
| 120 | ((2- (((3S,6S,7aS, 8aR,9aR)-3- (((R)-1-(1H- pyrazol-4- yl)pyrrolidin- 3- yl)carba- moyl)-5- oxodeca- hydro-1H- cyclopropa[d] pyrrolo[1,2- a]azocin-6- yl) carbamoyl) benzo[b]thio- phen-5- yl)difluoro- methyl)phos- phonic acid | 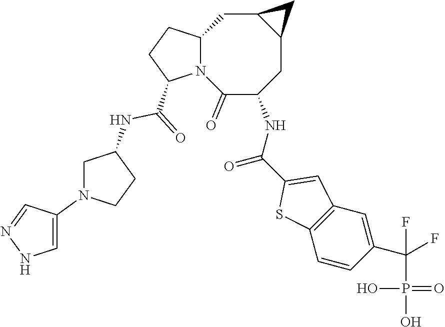 [SMILES: O=C(N[C@H]1C[C@@H]2C[C@@H]2C[C@H]2CC[C@@H](C(=O)N[C@@H]3CCN(c4cn[nH]c4)C3)N2C1=O)c1cc2cc(C(F)(F)P(=O)(O)O)ccc2s1] | 663.3 [M + H]⁺ | ¹H NMR (400 MHZ, CD₃OD) 88.19 (s, 1H), 8.12 (s, 1H), 7.95- 7.89 (m, 1H), 7.74 (d, J = 8.4 Hz, 1H), 7.12 (s, 2H), 4.45-4.38 (m, 1H), 4.30-4.24 (m, 1H), 3.18-3.13 (m, 1H), 3.03 (m, 1H), 2.99- 2.92 (m, 2H), 2.69 (s, 1H), 2.46-2.36 (m, 1H), 2.29 (m, 1H), 2.24- 2.17 (m, 2H), 2.04 (s, 1H), 1.97-1.86 (m, 2H), 1.74-1.64 (m, 1H), 1.31-1.27 (m, 2H), 1.00-0.87 (m, 3H), 0.69-0.60 (m, 1H), 0.06-- 0.01 (m, 1H). |
| 121 | (difluoro(2- (((3S,6S,7aR, 8aS,9aR)-5- ox0-3-(3- phenylaze- tidine-1- carbonyl)deca- hydro-1H- cyclopropa[d pyrrolo[1,2- a]azocin-6- yl)carbamoyl) benzo[b]thio- phen-5- yl)methyl) phosphonic acid | 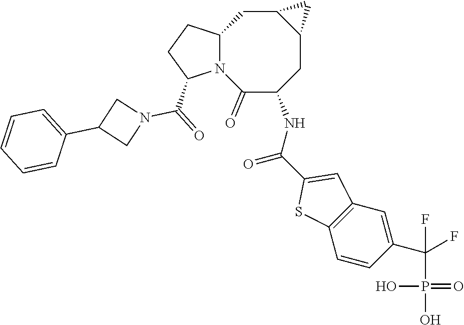 [SMILES: O=C(N[C@H]1C[C@H]2C[C@H]2C[C@H]2CC[C@@H](C(=O)N3CC(c4ccccc4)C3)N2C1=O)c1cc2cc(C(F)(F)P(=O)(O)O)ccc2s1] | 644.3 [M + H]⁺ | ¹H NMR (400 MHZ, CD₃OD) δ 8.97-8.81 (m, 1H), 8.33 (d, J = 5.2 Hz, 1H), 8.18-8.05 (m, 2H), 7.59 (d, J = 8.4 Hz, 1H), 7.39-7.34 (m, 3H), 7.34-7.30 (m, 1H), 7.30-7.20 (m, 1H), 4.86-4.80 (m, 1H), 4.58 (t, J = 8.4 Hz, 1H), 4.35-4.28 (m, 2H), 4.20-4.16 (m, 2H), 3.85 (s, 2H), 2.28- 2.15 (m, 2H), 2.10- 1.98 (m, 2H), 1.96- 1.84 (m, 2H), 1.74- 1.49 (m, 2H), 1.36- 1.16 (m, 2H), 0.84- 0.72 (m, 1H), 0.05 (q, J = 4.8 Hz, 1H). |
| 122 | (difluoro(2- (((3S,6S,7aR, 8aS,9aR)-3- ((2-methyl-2- (pyridin-3- yl)propyl) carbamoyl)-5- oxodeca- hydro-1H- cyclopropa[d] pyrrolo[1,2- a]azocin-6- yl)carbamoyl) benzo[b]thio- phen-5- yl)methyl) phosphonic acid | 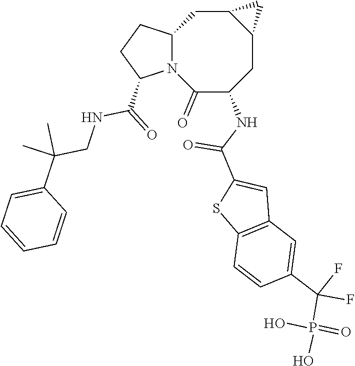 [SMILES: CC(C)(CNC(=O)[C@@H]1CC[C@@H]2C[C@@H]3C[C@@H]3C[C@H](NC(=O)c3cc4cc(C(F)(F)P(=O)(O)O)ccc4s3)C(=O)N21)c1ccccc1] | 661.3 [M + H]⁺ | ¹H NMR (400 MHZ, CD₃OD) δ 8.64 (d, J = 1.6 Hz, 1H), 8.48 (d, J = 4.8 Hz, 1H), 8.19 (s, 1H), 8.12 (t, J = 6.0 Hz, 1H), 8.05 (s, 1H), 7.85 (d, J = 8.8 Hz, 1H), 7.71 (d, J = 8.4 Hz, 1H), 7.62-7.53 (m, 1H), 4.94 (d, J = 4.0 Hz, 1H), 4.32-4.22 (m, 2H), 3.54-3.42 (m, 1H), 3.35 (d, J = 6.0 Hz, 1H), 2.48-2.33 (m, 1H), 2.14-2.03 (m, 3H), 1.92-1.80 (m, 2H), 1.69-1.51 (m, 2H), 1.5o-1.44 (m, 1H), 1.42 (s, 6H), 1.30- 1.19 (m, 1H), 0.89- 0.81 (m, 1H), 0.15 (d, J = 4.8 Hz, 1H). |
| 123 | (difluoro(2- (((3S,6S,7aR, 8aS,9aR)-3- ((3-hydroxy- 2-(pyridin-3- yl)propyl) carbamoyl)-5- oxodeca- hydro-1H- cyclopropa[d] pyrrolo[1,2- a]azocin-6- yl)carbamoyl) benzo[b]thio- phen-5- yl)methyl) phosphonic acid | 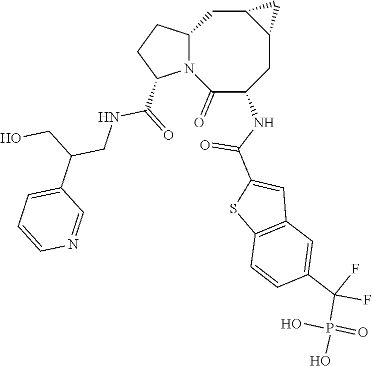 [SMILES: O=C(N[C@H]1C[C@H]2C[C@H]2C[C@H]2CC[C@@H](C(=O)NCC(CO)c3cccnc3)N2C1=O)c1cc2cc(C(F)(F)P(=O)(O)O)ccc2s1] | 662.3 [M + H]⁺ | ¹H NMR (400 MHZ, CD₃OD) δ 8.50-8.47 (m, 1H), 8.42 (d, J = 4.8 Hz, 1H), 8.18 (s, 1H), 8.11 (s, 1H), 7.97- 7.86 (m, 2H), 7.74 (d, J = 8.4 Hz, 1H), 7.50- 7.40 (m, 1H), 4.93 (d, J = 4.8 Hz, 1H), 4.33- 4.22 (m, 2H), 3.87- 3.74 (m, 2H), 3.63- 3.46 (m, 2H), 3.17- 3.03 (m, 1H), 2.38- 2.33 (m, 1H), 2.17- 2.00 (m, 3H), 1.99- 1.81 (m, 2H), 1.80- 1.55 (m, 2H), 1.52- 1.40 (m, 1H), 1.32- 1.20 (m, 1H), 0.90- 0.80 (m, 1H), 0.20- 0.16 (m, 1H). |
| 124 | ((2- (((3S,6S,7aR, 8aS,9aR)-3- (((1H- pyrrolo[2,3- b]pyridin-3- yl)methyl) carbamoyl)-5- oxodeca- hydro-1H- cyclopropa[d] pyrrolo[1,2- a]azocin-6- yl)carbamoyl) benzo[b]thio- phen-5- yl)difluoro- methyl)phos- phonic acid | 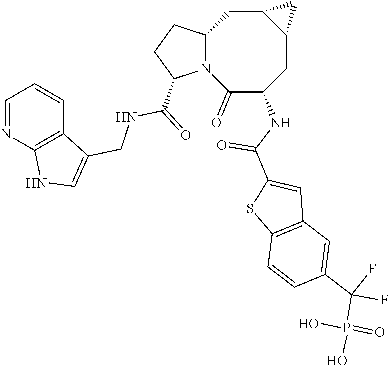 [SMILES: O=C(N[C@H]1C[C@H]2C[C@H]2C[C@H]2CC[C@@H](C(=O)NCc3c[nH]c4ncccc34)N2C1=O)c1cc2cc(C(F)(F)P(=O)(O)O)ccc2s1] | 658.1 [M + H]⁺ | ¹H NMR (400 MHZ, CD₃OD) δ 8.19-8.07 (m, 3H), 8.01 (s, 1H), 7.84 (d, J = 8.4 Hz, 1H), 7.71 (d, J = 8.8 Hz, 1H), 7.36 (d, J = 3.2 Hz, 1H), 7.16-7.05 (m, 1H), 4.63-4.54 (m, 1H), 4.52-4.42 (m, 1H), 4.41-4.26 (m, 2H), 2.44-2.32 (m, 1H), 2.29-2.10 (m, 3H), 2.09-1.97 (m, 1H), 1.97-1.85 (m, 2H), 1.82-1.70 (m, 1H), 1.46 (d, J = 3.6 Hz, 1H), 1.28 (d, J = 6.8 Hz, 1H), 0.90-0.81 (m, 1H), 0.25-0.17 (m, 1H). |
| 125 | (difluoro(2- (((3S,6S,7aR, 8aS,9aR)-5- ox0-3-(4- (tetrahydro- 2H-pyran-4- yl)pipera- zine-1- carbonyl) decahydro-1H- cyclopropa[d] pyrrolo[1,2- a]azocin-6- yl)carbamoyl) benzo[b]thio- phen-5- yl)methyl) phosphonic acid | 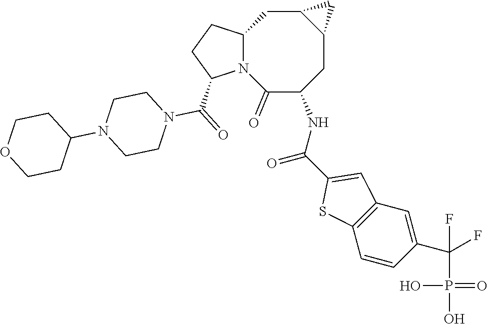 [SMILES: O=C(N[C@H]1C[C@H]2C[C@H]2C[C@H]2CC[C@@H](C(=O)N3CCN(C4CCOCC4)CC3)N2C1=O)c1cc2cc(C(F)(F)P(=O)(O)O)ccc2s1] | 681.2 [M + H]⁺ | ¹H NMR (400 MHZ, CD₃OD) δ 8.18 (s, 1H), 8.10 (s, 1H), 7.94 (d, J = 8.8 Hz, 1H), 7.74 (d, J = 8.0 Hz, 1H), 4.94 (d, J = 4.4 Hz, 1H), 4.40-4.28 (m, 1H), 3.98 (d, J = 8.4 Hz, 2H), 3.93-3.74 (m, 2H), 3.71-3.59 (m, 1H), 3.59-3.46 (m, 1H), 3.42-3.36 (m, 2H), 3.05-2.96 (m, 1H), 2.91-2.76 (m, 3H), 2.42-1.96 (m, 6H), 1.96-1.85 (m, 3H), 1.83-1.71 (m, 2H), 1.68-1.53 (m, 2H), 1.52-1.40 (m, 1H), 1.38-1.20 (m, 2H), 0.90-0.81 (m, 1H), 0.19 (d, J = 4.8 Hz, 1H). |
| 126 | ((2- (((3S,6S,7aR, 8aS,9aR)-3- (((3S,4S)-1- acetyl-4- fluoropy- rrolidin-3- yl)carba- moyl)-5- oxodeca- hydro-1H- cyclopropa[d] pyrrolo[1,2- a]azocin-6- yl)carbamoyl) benzo[b]thio- phen-5- yl)difluoro- methyl)phos- phonic acid | 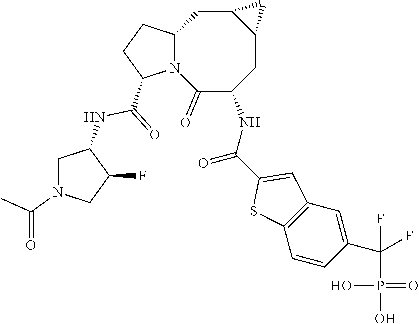 [SMILES: CC(=O)N1C[C@H](NC(=O)[C@@H]2CC[C@@H]3C[C@@H]4C[C@@H]4C[C@H](NC(=O)c4cc5cc(C(F)(F)P(=O)(O)O)ccc5s4)C(=O)N32)[C@@H](F)C1] | 657.3 [M + H]⁺ | ¹H NMR (400 MHZ, CD₃OD) δ 8.19 (s, 1H), 8.12-8.08 (m, 1H), 7.93 (m, 1H), 7.75 (m, 1H), 4.95-4.92 (m, 2H), 4.36 (s, 3H), 3.96- 3.81 (m, 1H), 3.79- 3.62 (m, 3H), 2.44- 2.32 (m, 1H), 2.25- 2.06 (m, 6H), 2.03- 1.90 (m, 2H), 1.89- 1.68 (m, 2H), 1.53- 1.41 (m, 1H), 1.35- 1.21 (m, 1H), 0.91- 0.82 (m, 1H), 0.27- 0.17 (m, 1H). |
| 127 | ((2- (((3S,6S,7aR, 8aS,9aR)-3- (((3S,4R)-1- acetyl-4- fluoropy- rrolidin-3- yl)carba- moyl)-5- oxodeca- hydro-1H- cyclopropa[d] pyrrolo[1,2- a]azocin-6- yl)carbamoyl) benzo[b]thio- phen-5- yl)difluoro- methyl)phos- phonic acid | 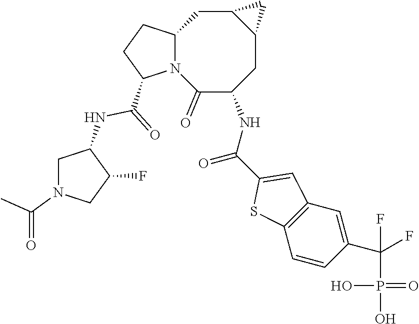 [SMILES: CC(=O)N1C[C@@H](F)[C@@H](NC(=O)[C@@H]2CC[C@@H]3C[C@@H]4C[C@@H]4C[C@H](NC(=O)c4cc5cc(C(F)(F)P(=O)(O)O)ccc5s4)C(=O)N32)C1] | 657.3 [M + H]⁺ | ¹H NMR (400 MHZ, CD₃OD) δ 8.18 (s, 1H), 8.10 (s, 1H), 7.93 (d, J = 8.4 Hz, 1H), 7.74 (d, J = 8.4 Hz, 1H), 5.23-5.05 (m, 1H), 4.95 (d, J = 4.0 Hz, 1H), 4.64-4.50 (m, 1H), 4.49-4.42 (m, 1H), 4.37-4.27 (m, 1H), 4.00-3.92 (m, 1H), 3.90-3.80 (m, 1H), 3.70-3.53 (m, 1H), 3.42 (m, 1H), 2.40- 2.32 (m, 1H), 2.30- 2.22 (m, 1H), 2.21- 2.12 (m, 2H), 2.08- 2.03 (m, 3H), 2.01- 1.85 (m, 3H), 1.81- 1.71 (m, 1H), 1.54- 1.42 (m, 1H), 1.35- 1.24 (m, 1H), 0.89- 0.81 (m, 1H), 0.19 (m, 1H). |
| 128 | (difluoro(2- (((3S,6S,7aR, 8aS,9aR)-3- (((3S,4S)-4- fluoro-1- (pyridin-3- yl)pyrrolidin- 3- yl)carba moyl)-5- oxodeca- hydro-1H- cyclopropa[d] pyrrolo[1,2- a]azocin-6- yl)carbamoyl) benzo[b]thio- phen-5- yl)methyl) phosphonic acid | 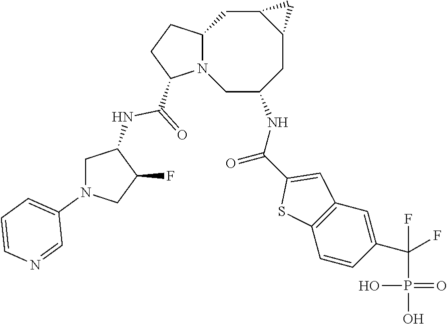 [SMILES: O=C(N[C@H]1C[C@H]2C[C@H]2C[C@H]2CC[C@@H](C(=O)N[C@H]3CN(c4cccnc4)C[C@@H]3F)N2C1)c1cc2cc(C(F)(F)P(=O)(O)O)ccc2s1] | 692.4 [M + H]⁺ | ¹H NMR (400 MHZ, CD₃OD) δ 7.30 (s, 1H), 7.10-7.04 (m, 2H), 7.01-6.94 (m, 2H), 6.63-6.59 (m, 1H), 6.49-6.47 (m, 1H), 4.58-4.42 (m, 1H), 3.85-3.81 (m, 1H), 3.68-3.57 (m, 2H), 3.04-2.97 ( m, 1H), 2.89-2.78 (m, 1H), 2.73 (d, J = 10.4 Hz, 1H), 2.63-2.62 (m, 7H), 1.77-1.72 (m, 1H), 1.63-1.56 (m, 1H), 1.53-1.42 (m, 2H), 1.33-1.17 (m, 3H), 1.05-0.95 (m, 1H), 0.79-0.73 (m, 1H), 0.63-0.58 (m, 1H), 0.22-0.17 (m, 1H),-0.45-- 0.48 (m, 1H). |
| 129 | (difluoro(2- (((3S,6S,7aR, 8aS,9aR)-3- (((3S,4R)-4- fluoro-1- (pyridin-3- yl)pyrrolidin- 3- yl)carba- moyl)-5- oxodeca- hydro-1H- cyclopropa[d] pyrrolo[1,2- a]azocin-6- yl)carbamoyl) benzo[b]thio- phen-5- yl)methyl) phosphonic acid | 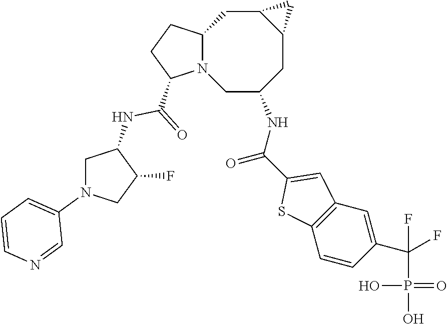 [SMILES: O=C(N[C@H]1C[C@H]2C[C@H]2C[C@H]2CC[C@@H](C(=O)N[C@H]3CN(c4cccnc4)C[C@H]3F)N2C1)c1cc2cc(C(F)(F)P(=O)(O)O)ccc2s1] | 692.2 [M + H]⁺ | ¹H NMR (400 MHZ, CD₃OD) δ 8.76 (d, J = 7.2 Hz, 1H), 8.53 (d, J = 7.6 Hz, 1H), 8.28 (s, 1H), 8.15-7.98 (m, 4H), 7.59 (d, J = 8.4 Hz, 2H), 7.43 (d, J = 8.4 Hz, 1H), 5.36-5.20 (m, 1H), 4.82 (t, J = 5.2 Hz, 1H), 4.64-4.56 (m, 1H), 4.44 (d, J = 8.5 Hz, 1H), 4.20 (s, 1H), 3.73-3.69 (m, 2H), 3.64 (s, 1H), 3.21 (t, J = 9.6 Hz, 1H), 2.30- 2.15 (m, 2H), 2.10- 2.00 (m, 2H), 1.95- 1.81 (m, 2H), 1.76- 1.59 (m, 2H), 1.39- 1.29 (m, 1H), 1.27- 1.20 (m, 1H), 0.81- 0.75 (m, 1H), 0.07- 0.01 (m, 1H). |

### Phosphonate Esters and Amides Data Tables:

### General Scheme 1: Phosphonate Esters or Amides

The following compounds in Table 28 were prepared according to the representative procedure described above for the synthesis of isopropyl ((difluoro(2-(((3S,6S,7aS,8aR,9aR)-3-(3-(morpholine-4-carbonyl)azetidine-1-carbonyl)-5-oxodecahydro-1H-cyclopropa[d]pyrrolo[1,2-a]azocin-6-yl)carbamoyl)benzo[b]thiophen-5-yl)methyl)(phenoxy)phosphoryl)glycinate (Example 2) utilizing the appropriate starting materials and modifications.

TABLE 28

| Com- | | | | |
|---|---|---|---|---|
| pound | Name | Structure | LCMS | NMR |
| 130 | ((((2- (((3S,6S,7aS, 8aR,9aR)-3- (3- (morpholine- 4-carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) phosphoryl)bis (oxy))bis (methylene) bis(tetrahydro- 2H-pyran-4-yl) bis(carbonate) | 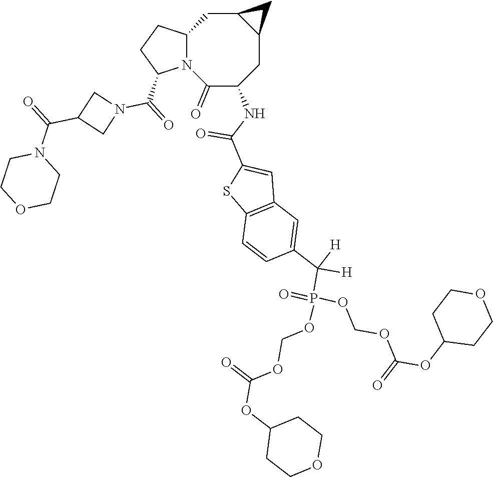 [SMILES: O=C(OCOP(=O)(Cc1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1)OCOC(=O)OC1CCOCC1)OC1CCOCC1] | 961 [M + H]⁺ | ¹H NMR (400 MHz, CDCl₃) δ 7.83-7.70 (m, 3H), 7.40-7.29 (m, 2H), 5.71-5.59 (m, 4H), 5.12-4.98 (m, 1H), 4.90-4.70 (m, 3H), 4.60-4.39 (m, 2H), 4.37-4.26 (m, 1H), 4.22-4.04 (m, 2H), 3.95-3.86 (m, 4H), 3.72-3.64 (m, 5H), 3.56-3.50 (m, 4H), 3.44-3.35 (m, 2H), 3.32-3.25 (m, 2H), 2.34-2.25 (m, 3H), 2.17-2.07 (m, 2H), 2.04-1.94 (m, 6H), 1.77-1.69 (m, 6H), 1.48-1.40 (m, 1H), 1.00-0.91 (m, 1H), 0.89-0.77 (m, 1H). |
| 131 | S,S′- ((((difluoro(2- (((3S,6S,7aS, 8aR,9aR)-3- (3- (morpholine- 4- carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) phosphoryl) bis(oxy)) bis(ethane- 2,1-diyl)) bis(3- hydroxy-2,2- dimethyl- propane- thioate) | 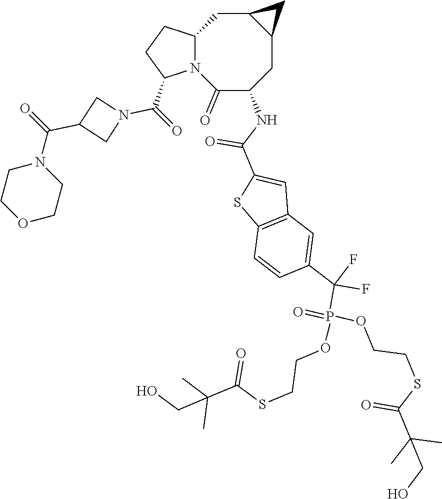 [SMILES: CC(C)(CO)C(=O)SCCOP(=O)(OCCSC(=O)C(C)(C)CO)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 1001 [M + H]⁺ | ¹H NMR (400 MHz, DMSO-d₆) δ 9.03- 8.87 (m, 1H), 8.35 (s, 1H), 8.23-8.18 (m, 1H), 8.18-8.13 (m, 1H), 7.63-7.57 (m, 1H), 4.96-4.89 (m, 2H), 4.66-4.59 (m, 1H), 4.54-4.48 (m, 1H), 4.38-4.25 (m, 4H), 4.20-4.11 (m, 6H), 4.02-3.84 (m, 8H), 3.53-3.52 (m, 2H), 3.11-3.09 (m, 4H), 2.05-2.02 (m, 2H), 1.84-1.80 (m, 2H), 1.65-1.60 (m, 2H), 1.38-1.31 (m, 2H), 1.12-1.08 (m, 12H), 0.80-0.77 (m, 1H), 0.74-0.70 (m, 1H), 0.05-−0.05 (m, 2H). |
| 132 | (((difluoro(2- (((3S,6S,7aS, 8aR,9aR)-3- (3- (morpholine- 4-carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) phosphoryl) bis(oxy)) bis(methylene) bis(3- hydroxy-2,2- dimethyl- propanoate) | 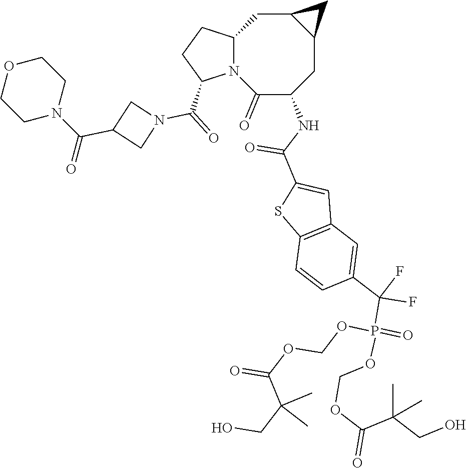 [SMILES: CC(C)(CO)C(=O)OCOP(=O)(OCOC(=O)C(C)(C)CO)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 940.9 [M + H]⁺ | ¹H NMR (400 MHz, CDCl₃) δ 8.07 (s, 1H), 8.01-7.85 (m, 2H), 7.66-7.44 (m, 2H), 5.80-5.64 (m, 4H), 5.38-4.95 (m, 2H), 4.80-4.71 (m, 1H), 4.58-4.52 (m, 1H), 4.45-4.38 (m, 1H), 4.34-4.24 (m, 1H), 4.22-4.15 (m, 1H), 4.13-4.03 (m, 1H), 3.74-3.57 (m, 12H), 3.32-3.25 (m, 2H), 2.37-2.16 (m, 6H), 1.69-1.64 (m, 2H), 1.22-1.17 (m, 12H), 0.97-0.92 (m, 1H), 0.87-0.82 (m, 1H). (TFA salt). |
| 133 | S,S′-(((((2- (((3S,6S,7aS, 8aR,9aR)-3- (3- (morpholine- 4-carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) phosphoryl) bis(oxy)) bis(ethane- 2,1-diyl)) bis(3- hydroxy-2,2- dimethyl- propane- thioate | 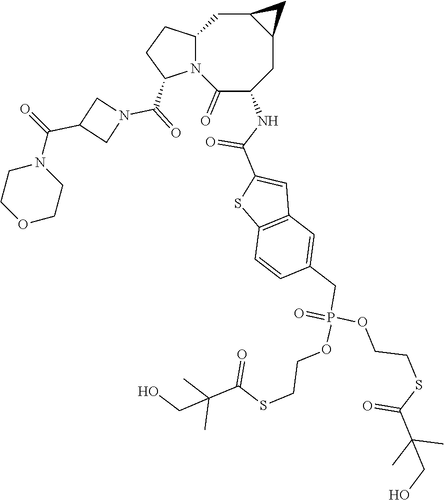 [SMILES: CC(C)(CO)C(=O)SCCOP(=O)(Cc1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1)OCCSC(=O)C(C)(C)CO] | 965 [M + H]⁺ | ¹H NMR (400 MHz, DMSO-d₆) δ 8.86- 8.75 (m, 1H), 8.23 (s, 1H), 7.97-7.92 (m, 1H), 7.85 (s, 1H), 7.41- 7.35 (m, 1H), 4.97- 4.89 (m, 1H), 4.66- 4.49 (m, 1H), 4.36- 4.27 (m, 2H), 4.19- 4.12 (m, 1H), 4.00- 3.95 (m, 4H), 3.92- 3.84 (m, 1H), 3.74- 3.63 (m, 2H), 3.54- 3.51 (m, 4H), 3.43- 3.42 (m, 4H), 3.33- 3.30 (m, 3H), 3.28- 3.24 (m, 3H), 3.06- 3.02 (m, 4H), 2.36- 2.30 (m, 1H), 2.23- 1.94 (m, 6H), 1.89- 1.74 (m, 2H), 1.67- 1.57 (m, 1H), 1.10 (s, 12H), 0.81-0.67 (m, 2H). |
| 134 | isopropyl (((2- (((3S,6S,7aS, 8aR,9aR)-3- (3- (morpholine-4- carbonyl) azetidine-1- carbonyl)-5- oxodecahydro- 1H- cyclopropa[d] pyrrolo[1,2-a] azocin-6- yl)carbamoyl) benzo[b] thiophen-5- yl)methyl) (phenoxy) phosphoryl)-L- phenyl- alaninate | 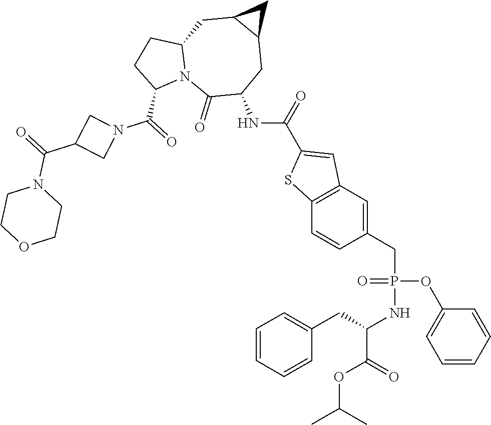 [SMILES: CC(C)OC(=O)[C@H](Cc1ccccc1)NP(=O)(Cc1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1)Oc1ccccc1] | 910.2 [M + H]⁺ | ¹H NMR (400 MHz, DMSO-d₆) δ 8.89- 8.71 (m, 1H), 8.22 (d, J = 3.3 Hz, 1H), 7.92 (t, J = 8.1 Hz, 1H), 7.81 (d, J = 7.1 Hz, 1H), 7.36 (t, J = 7.6 Hz, 1H), 7.28 (t, J = 7.8 Hz, 2H), 7.24- 7.15 (m, 3H), 7.14- 6.99 (m, 5H), 5.69- 5.67 (m, 1H), 4.98- 4.85 (m, 1H), 4.74- 4.48 (m, 2H), 4.37- 4.25 (m, 2H), 4.19- 4.10 (m, 1H), 4.05- 3.84 (m, 3H), 3.76- 3.61 (m, 1H), 3.57- 3.49 (m, 4H), 3.48- 3.42 (m, 2H), 3.28- 3.22 (m, 2H), 3.17- 2.99 (m, 1H), 2.83- 2.72 (m, 1H), 2.69- 2.53 (m, 1H), 2.37- 2.25 (m, 1H), 2.18- 1.93 (m, 4H), 1.90- 1.75 (m, 2H), 1.69- 1.56 (m, 1H), 1.38- |
| | | | | 1.29 (m, 1H), 1.06- |
| | | | | 0.89 (m, 6H), 0.83- |
| | | | | 0.67 (m, 2H), 0.03- |
| | | | | −0.05 (m, 1H). |
| 135 | ((((2- (((3S,6S,7aR, 8aS,9aR)-5- oxo-3-(3- (pyridin-3- yl)azetidine-1- carbonyl) decahydro-1H- cyclopropa[d] pyrrolo[1,2- a]azocin-6- yl)carbamoyl) benzo[b] thiophen-5- yl)methyl) phosphoryl) bis(oxy)) bis(methylene) bis(tetrahydro- 2H-pyran- 4-yl) bis(carbonate) | 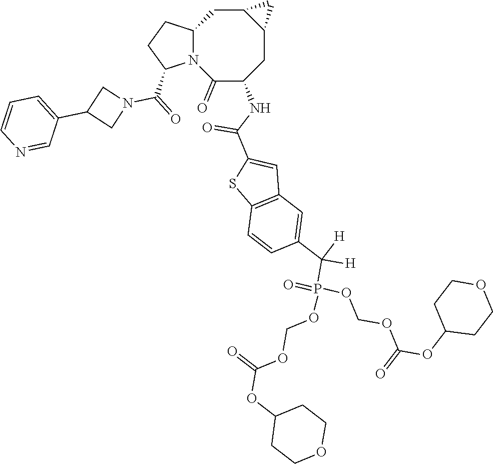 [SMILES: O=C(OCOP(=O)(Cc1ccc2sc(C(=O)N[C@H]3C[C@H]4C[C@H]4C[C@H]4CC[C@@H](C(=O)N5CC(c6cccnc6)C5)N4C3=O)cc2c1)OCOC(=O)OC1CCOCC1)OC1CCOCC1] | 925.0 [M + H]⁺ | ¹H NMR (400 MHz, CDCl₃) δ 8.74-8.58 (m, 2H), 8.02-8.00 (m, 1H), 7.91 (s, 1H), 7.82- 7.70 (m, 3H), 7.50- 7.30 (m, 2H), 5.62 (ddd, J = 11.3, 6.7, 3.3 Hz, 4H), 5.70-490 (m, 1H), 4.86-4.77 (m, 2H), 4.63 (d, J = 9.1 Hz, 1H), 4.40-4.38 (m, 2H), 4.22-4.18 (m, 2H), 4.10-4.05 (m, 1H), 3.94-3.89 (m, 4H), 3.53-3.50 (m, 4H), 3.43-3.40 (m, 2H), 2.35-2.33 (m, 1H), 2.23-2.06 (m, 6H), 2.00-1.92 (m, 6H), 1.74-1.69 (m, 3H), 1.25-1.23 (m, 2H), 0.85-0.81 (m, 1H), 0.30-0.20 (m, 1H). |
| 136 | S,S′-(((((2- (((3S,6S,7aS, 8aR,9aR)-3- (3-(1H- imidazol-1- yl)azetidine-1- carbonyl)-5- oxodecahydro- 1H- cyclopropa[d] pyrrolo[1,2- a]azocin-6- yl)carbamoyl) benzo[b] thiophen-5- yl)difluoro- methyl) phosphoryl) bis(oxy))bis (ethane-2,1- diyl))bis(3- hydroxy-3- methylbutane thioate) | 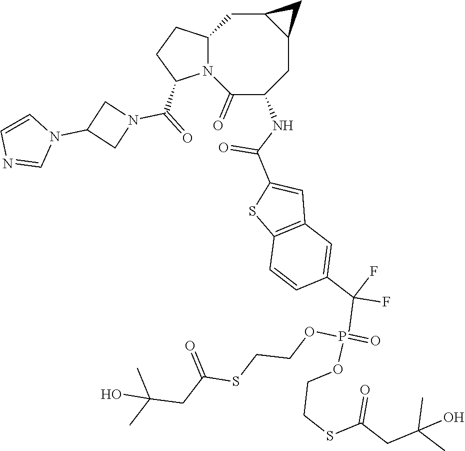 | 954.3 [M + H]⁺ | ¹H NMR (400 MHz, DMSO-d₆) δ 9.00 (dd, J = 30.8, 7.1 Hz, 1H), 8.37 (s, 1H), 8.27- 8.15 (m, 2H), 8.14- 7.97 (m, 1H), 7.66- 7.39 (m, 2H), 7.09 (d, J = 34.2 Hz, 1H), 5.28- 5.13 (m, 1H), 5.00- 4.65 (m, 3H), 4.40- 4.28 (m, 2H), 4.25- 4.08 (m, 5H), 4.06- 3.98 (m, 1H), 3.15- 3.04 (m, 4H), 2.66 (s, 4H), 2.40-2.29 (m, 1H), 2.20-1.81 (m, 6H), 1.72-1.59 (m, 1H), 1.39-1.28 (m, 1H), 1.16 (s, 12H), 0.87-0.68 (m, 2H), 0.07-−0.00 (m, 1H). (TFA salt) |
| 137 | S,S′-(((((2- (((3S,6S,7aS, 8aR,9aR)-3- (3-(1H- imidazol-1-yl) azetidine-1- carbonyl)-5- oxodecahydro- 1H- cyclopropa[d] pyrrolo[1,2- a]azocin-6- yl)carbamoyl) benzo[b] thiophen-5-yl) difluoro- methyl) phosphoryl)bis (oxy)) bis(ethane- 2,1-diyl))bis(3- hydroxy-2,2- dimethylpro- panethioate) | 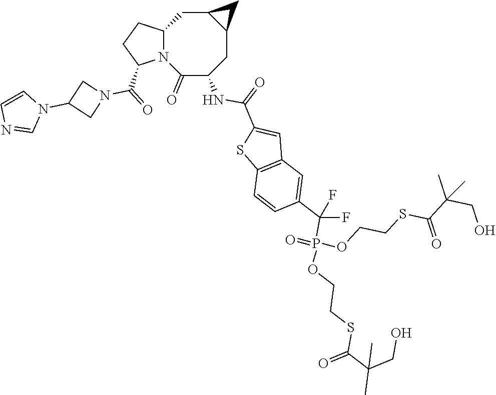 [SMILES: CC(C)(CO)C(=O)SCCOP(=O)(OCCSC(=O)C(C)(C)CO)C(F)(F)c1ccc2sc(C(=O)N[C@H]3C[C@@H]4C[C@@H]4C[C@H]4CC[C@@H](C(=O)N5CC(n6ccnc6)C5)N4C3=O)cc2c1] | 953.8 [M + H]⁺ | ¹H NMR (400 MHz, CDCl₃) δ 9.59 (s, 1H), 8.48-7.79 (m, 4H), 7.66-7.38 (m, 3H), 5.41-5.29 (m, 1H), 5.26-5.08 (m, 2H), 4.88-4.81 (m, 1H), 4.56-4.46 (m, 2H), 4.43-4.34 (m, 2H), 4.26-4.18 (m, 4H), 3.57 (s, 4H), 3.21- 3.03 (m, 4H), 2.54- 2.27 (m, 3H), 2.23- 2.15 (m, 1H), 2.13- 1.91 (m, 3H), 1.77- 1.61 (m, 1H), 1.38- 1.32 (m, 1H), 1.28- 1.12 (m, 12H), 0.93- 0.80 (m, 2H), 0.08- 0.00 (m, 1H). |
| 138 | S-(2- (((difluoro(2- (((3S,6S,7aS, 8aR,9aR)-3-(3- (morpholine-4- carbonyl) azetidine-1- carbonyl)-5- oxodecahydro- 1H-cyclopropa [d]pyrrolo[1,2- a]azocin-6-yl) carbamoyl)benzo [b]thiophen-5- yl)methyl)(2- (pivaloylthio) ethoxy) phosphoryl) | 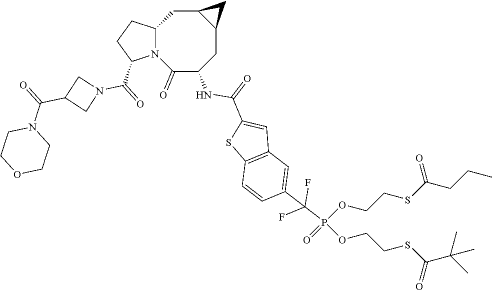 [SMILES: CCCC(=O)SCCOP(=O)(OCCSC(=O)C(C)(C)C)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 955 [M + H]⁺ | ¹H NMR (400 MHz, DMSO-d₆) δ 9.03- 8.91 (m, 1H), 8.36 (s, 1H), 8.23-8.14 (m, 2H), 7.62-7.56 (m, 1H), 4.99-4.89 (m, 1H), 4.67-4.50 (m, 1H), 4.39-4.28 (m, 2H), 4.21-4.11 (m, 5H), 4.04-3.87 (m, 2H), 3.74-3.64 (m, 1H), 3.57-3.51 (m, 4H), 3.48-3.41 (m, 2H), 3.29-3.22 (m, 2H), 3.16-3.07 (m, 4H), 2.54 (t, J = 7.3 Hz, 2H), 2.37-2.27 (m, 1H), 2.14-1.96 (m, |
| | oxy)ethyl) | | | 4H), 1.90-1.77 (m, |
| | butanethioate | | | 2H), 1.67-1.59 (m, |
| | | | | 1H), 1.59-1.52 (m, |
| | | | | 2H), 1.40-1.32 (m, |
| | | | | 1H), 1.16 (s, 9H), 0.86 |
| | | | | (t, J = 7.4 Hz, 3H), |
| | | | | 0.82-0.75 (m, 1H), |
| | | | | 0.74-0.68 (m, 1H), |
| | | | | 0.02-−0.04 (m, 1H). |
| 139 | ((((2- (((3S,6S,7aR, 8aS,9aR)-3- ((rel-trans)- 3-cyano-4- phenyl- pyrrolidine-1- carbonyl)-5- oxodecahydro- 1H-cyclopropa [d]pyrrolo[1,2- a]azocin-6- yl)carbamoyl) benzo[b] thiophen-5- yl)difluoro- methyl) phosphoryl) bis(oxy)) bis(methylene) bis(2,2- dimethylpro- panoate) | 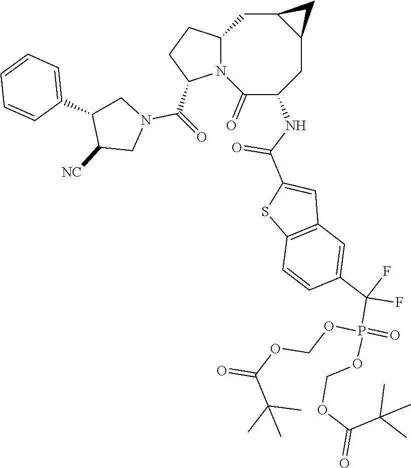 [SMILES: CC(C)(C)C(=O)OCOP(=O)(OCOC(=O)C(C)(C)C)C(F)(F)c1ccc2sc(C(=O)N[C@H]3C[C@@H]4C[C@@H]4C[C@H]4CC[C@@H](C(=O)N5C[C@@H](C#N)[C@H](c6ccccc6)C5)N4C3=O)cc2c1] | 911.4 [M + H]⁺ | ¹H NMR (400 MHz, DMSO-d₆) δ 8.96 (dd, J = 14.5, 7.5 Hz, 1H), 8.38-8.26 (m, 1H), 8.25-8.16 (m, 1H), 8.12 (s, 1H), 7.63- 7.51 (m, 1H), 7.47- 7.28 (m, 5H), 5.81- 5.64 (m, 4H), 5.01- 4.90 (m, 1H), 4.62- 4.35 (m, 1H), 4.24- 4.14 (m, 1H), 4.00- 3.81 (m, 1H), 3.73- 3.64 (m, 1H), 3.59- 3.45 (m, 1H), 2.41- 2.31 (m, 1H), 2.21- 2.00 (m, 4H), 1.93- 1.82 (m, 2H), 1.72- 1.60 (m, 1H), 1.45- 1.35 (m, 1H), 1.12 (s, 18H), 0.89-0.79 (m, 4H), 0.78-0.71 (m, 1H), 0.01-−0.01 (m, 1H). |
| 140 | ((((2- (((3S,6S,7aR, 8aS,9aR)-3- ((3S,4R)-3- cyano-4-phenyl- pyrrolidine-1- carbonyl)-5- oxodecahydro- 1H-cyclopropa [d]pyrrolo[1,2- a]azocin-6- yl)carbamoyl) benzo[b] thiophen-5-yl) difluoromethyl) phosphoryl) bis(oxy)) bis(methylene) bis(2,2- dimethylpro- panoate) | 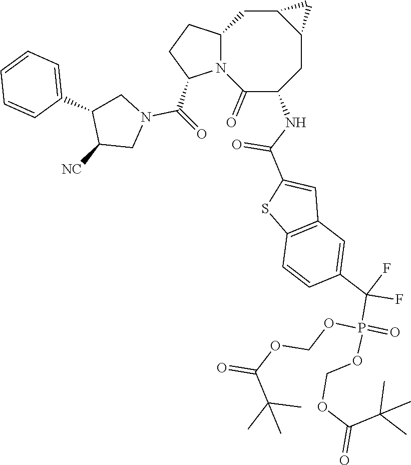 [SMILES: CC(C)(C)C(=O)OCOP(=O)(OCOC(=O)C(C)(C)C)C(F)(F)c1ccc2sc(C(=O)N[C@H]3C[C@H]4C[C@H]4C[C@H]4CC[C@@H](C(=O)N5C[C@@H](C#N)[C@H](c6ccccc6)C5)N4C3=O)cc2c1] | 911.4 [M + H]⁺ | ¹H NMR (400 MHz, CDCl₃) δ 8.13-8.02 (m, 1H), 8.00-7.75 (m, 3H), 7.68-7.56 (m, 1H), 7.46-7.29 (m, 5H), 5.86-5.52 (m, 4H), 5.50-4.83 (m, 1H), 4.70-4.28 (m, 2H), 4.24-3.98 (m, 2H), 3.87-3.54 (m, 2H), 3.39-3.12 (m, 1H), 2.83 (s, 1H), 2.35-1.74 (m, 9H), 1.30-1.12 (m, 19H), 0.90-0.76 (m, 1H), 0.35-0.12 (m, 1H). (free base). |
| 141 | S-(2- (((difluoro(2- (((3S,6S,7aS, 8aR,9aR)-3-(3- (morpholine-4- carbonyl) azetidine-1- carbonyl)-5- oxodecahydro- 1H-cyclopropa [d]pyrrolo[1,2- a]azocin-6- yl)carbamoyl) benzo[b] thiophen-5-yl) methyl)(((iso- propoxy- | 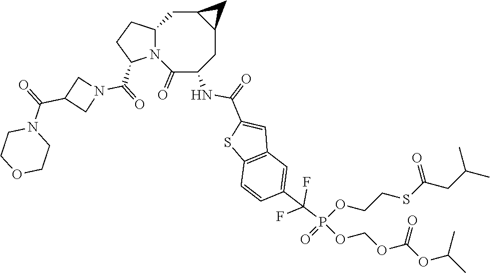 [SMILES: CC(C)CC(=O)SCCOP(=O)(OCOC(=O)OC(C)C)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 941.3 [M + H]⁺ | ¹H NMR (400 MHz, CDCl₃) δ 8.18-8.03 (m, 1H), 7.94 (d, J = 8.6 Hz, 1H), 7.85 (s, 1H), 7.72-7.60 (m, 1H), 7.44-7.34 (m, 1H), 5.77-5.57 (m, 2H), 5.11-4.86 (m, 2H), 4.82-4.37 (m, 2H), 4.37-3.98 (m, 5H), 3.75-3.54 (m, 6H), 3.35-3.21 (m, 2H), 3.20-3.04 (m, 2H), 2.42 (d, J = 7.1 Hz, 2H), 2.36-2.07 (m, 5H), 2.06-1.99 (m, 2H), 1.73-1.52 |
| | carbonyl)oxy) | | | (m, 5H), 1.50-1.39 |
| | methoxy)phos- | | | (m, 1H), 1.32 (d, J = |
| | phoryl)oxy) | | | 6.3 Hz, 6H), 1.28- |
| | ethyl) 3- | | | 1.24 (m, 1H), 0.94 (d, |
| | methylbutane | | | J = 6.7 Hz, 6H), 0.86- |
| | thioate | | | 0.78 (m, 1H). |
| 142 | S,S′-(((((2- (((3S,6S,7aR, 8aS,9aR)-3- (((3S,4R)-1- acetyl-4- fluoropyrrolidin- 3-yl) carbamoyl)-5- oxodecahydro- 1H-cyclopropa [d]pyrrolo[1,2- a]azocin-6- yl)carbamoyl) benzo[b] thiophen-5-yl) difluoromethyl) phosphoryl) bis(oxy)) bis(ethane-2,1- diyl)) bis(3- methylbutane thioate) | 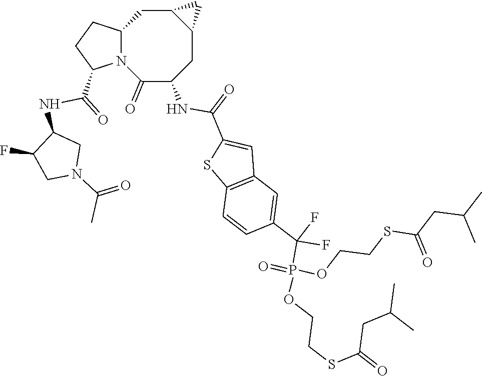 [SMILES: CC(=O)N1C[C@@H](F)[C@@H](NC(=O)[C@@H]2CC[C@@H]3C[C@@H]4C[C@@H]4C[C@H](NC(=O)c4cc5cc(C(F)(F)P(=O)(OCCSC(=O)CC(C)C)OCCSC(=O)CC(C)C)ccc5s4)C(=O)N32)C1] | 945.3 [M + H]⁺ | ¹H NMR (400 MHz, DMSO-d₆) δ 8.92 (m, 1H), 8.41 (dd, J = 15.7, 7.4 Hz, 1H), 8.34 (d, J = 3.1 Hz, 1H), 8.22 (d, J = 8.5 Hz, 1H), 8.16 (s, 1H), 7.59 (d, J = 8.5 Hz, 1H), 5.29-4.98 (m, 1H), 4.92-4.75 (m, 1H), 4.51-4.09 (m, 7H), 3.82-3.37 (m, 3H), 3.28-2.99 (m, 5H), 2.44 (d, J = 7.1 Hz, 4H), 2.29- 2.10 (m, 2H), 2.10- 1.97 (m, 4H), 1.94 (d, J = 16.5 Hz, 3H), 1.88- 1.82 (m, 1H), 1.74- 1.54 (m, 2H), 1.38- 1.16 (m, 2H), 1.01- 0.98 (m, 1H), 0.87 (d, J = 6.7 Hz, 12H), 0.81- 0.70 (m, 1H), 0.11-0.01 (m, 1H). |
| 143 | benzyl (((2- (((3S,6S,7aS, 8aR,9aR)-3-(3- (morpholine-4- carbonyl) azetidine-1- carbonyl)-5- oxodecahydro- 1H-cyclopropa [d]pyrrolo[1,2- a]azocin-6- yl)carbamoyl) benzo[b] thiophen-5- yl)methyl) (phenoxy) phosphoryl)-D- alaninate | 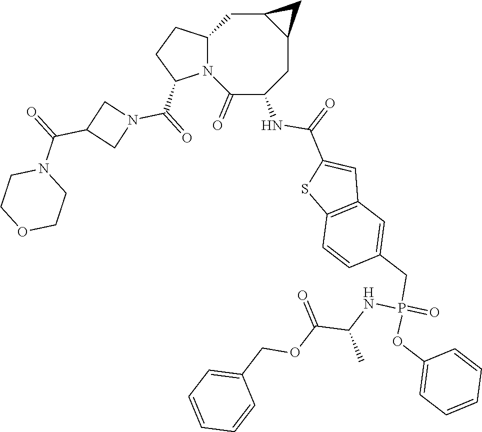 [SMILES: C[C@@H](NP(=O)(Cc1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1)Oc1ccccc1)C(=O)OCc1ccccc1] | 882.0 [M + H]⁺ | ¹H NMR (400 MHz, CDCl₃) δ 8.06-7.69 (m, 3H), 7.62-7.31 (m, 5H), 7.30-7.27 (m, 1H), 7.26-7.21 (m, 3H), 7.19-6.99 (m, 3H), 5.13-4.92 (m, 3H), 4.86-3.90 (m, 7H), 3.72-3.53 (m, 6H), 3.49-3.08 (m, 5H), 2.37-2.24 (m, 3H), 2.16-2.00 (m, 4H), 1.73-1.62 (m, 1H), 1.50-1.37 (m, 1H), 1.22-1.10 (m, 3H), 1.01-0.89 (m, 1H), 0.88-0.77 (m, 1H), 0.04-0.00 (m, 1H). |
| 144 | S,S′-(((((2- (((3S,6S,7aS, 8aR,9aR)-3-(3- (morpholine-4- carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) phosphoryl) bis(oxy)) bis(ethane-2,1- diyl)) bis(2,2- dimethyl- propanethioate) | 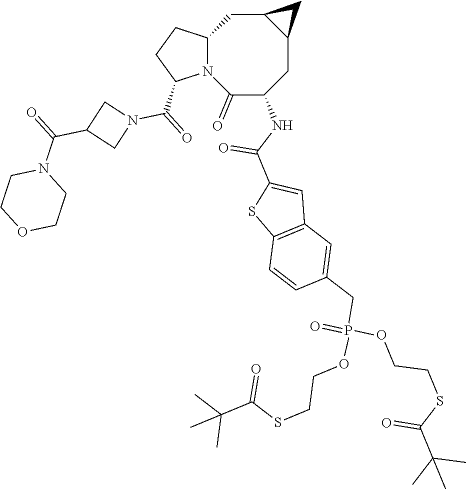 [SMILES: CC(C)(C)C(=O)SCCOP(=O)(Cc1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1)OCCSC(=O)C(C)(C)C] | 933.4 [M + H]⁺ | ¹H NMR (400 MHz, CDCl₃) δ 7.80-7.76 (m, 3H), 7.38-7.26 (m, 2H), 5.05-5.04 (m, 1H), 4.77 (t, J = 8.0 Hz, 1H), 4.53-4.29 (m, 3H), 4.16-4.02 (m, 6H), 3.69-3.58 (m, 7H), 3.33-3.27 (m, 4H), 3.06-3.04 (m, 4H), 2.31-2.28 (m, 3H), 2.09-2.03 (m, 4H), 1.72-1.63 (m, 2H), 1.44-1.37 (m, 1H), 1.22 (s, 18H), 0.98-0.92 (m, 1H), 0.84-0.78 (m, 1H). |
| 145 | (((difluoro(2- (((3S,6S,7aS, 8aR,9aR)-3-(3- (morpholine-4- carbonyl) azetidine-1- carbonyl)-5- oxodecahydro- 1H-cyclopropa [d]pyrrolo[1,2- a]azocin-6- yl)carbamoyl) benzo[b] thiophen-5- yl)methyl) phosphoryl) bis(oxy)) bis(methylene) bis(2,2- dimethyl- propanoate) | 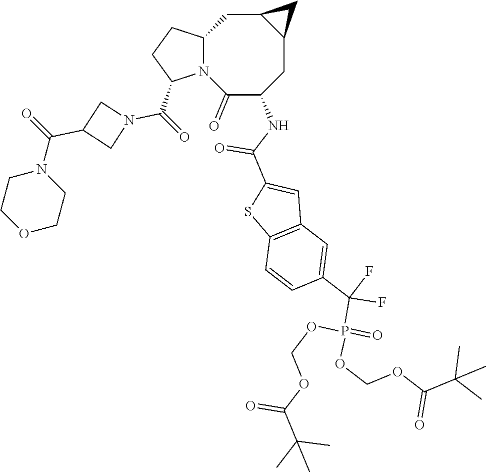 [SMILES: CC(C)(C)C(=O)OCOP(=O)(OCOC(=O)C(C)(C)C)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 909.4 [M + H]⁺ | ¹H NMR (400 MHz, CDCl₃) δ 8.08 (s, 1H), 7.97-7.79 (m, 2H), 7.69-7.36 (m, 2H), 5.79-5.60 (m, 4H), 5.12-5.00 (m, 1H), 4.99-4.72 (m, 1H), 4.59-4.27 (m, 3H), 4.22-4.04 (m, 2H), 3.74-3.53 (m, 7H), 3.29 (s, 2H), 2.39- 2.23 (m, 3H), 2.17- 1.98 (m, 4H), 1.72- 1.62 (m, 1H), 1.33- 1.26 (m, 1H), 1.20 (s, 18H), 0.98-0.91 (m, 1H), 0.85-0.82 (m, 1H), 0.04-0.00 (m, 1H). |
| 146 | S,S′-(((((2- (((3S,6S,7aS, 8aR,9aR)-3-(3- (1H-imidazol-1- yl)azetidine-1- carbonyl)-5- oxodecahydro- 1H- cyclopropa[d] pyrrolo[1,2- a]azocin-6- yl)carbamoyl) benzo[b] thiophen-5- yl)methyl) phosphoryl) bis(oxy)) bis(ethane-2,1- diyl)) bis(3- hydroxy-2,2- dimethyl- propanethioate) | 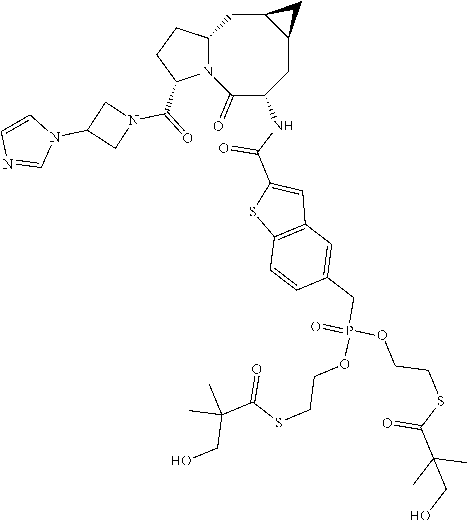 [SMILES: CC(C)(CO)C(=O)SCCOP(=O)(Cc1ccc2sc(C(=O)N[C@H]3C[C@@H]4C[C@@H]4C[C@H]4CC[C@@H](C(=O)N5CC(n6ccnc6)C5)N4C3=O)cc2c1)OCCSC(=O)C(C)(C)CO] | 918 [M + H]⁺ | ¹H NMR (400 MHz, DMSO-d₆) δ 9.41- 9.25 (m, 1H), 8.90- 8.77 (m, 1H), 8.27- 8.20 (m, 1H), 8.06- 8.02 (m, 1H), 7.99- 7.90 (m, 1H), 7.85 (s, 1H), 7.81-7.69 (m, 1H), 7.41-7.35 (m, 1H), 5.41-5.29 (m, 1H), 5.05-4.98 (m, 1H), 4.97-4.44 (m, 2H), 4.41-4.28 (m, 2H), 4.23-4.11 (m, 2H), 4.03-3.96 (m, 4H), 3.47-3.46 (m, 2H), 3.43-3.42 (m, 4H), 3.09-3.01 (m, 4H), 2.39-2.31 (m, 1H), 2.05-1.84 (m, 4H), 1.71-1.60 (m, 1H), 1.36-1.29 (m, 1H), 1.27-1.22 (m, 2H), 1.10 (s, 12H), 0.88-0.81 (m, 1H), 0.78-0.69 (m, 1H), 0.08-0.01 (m, 1H). |
| 147 | diisopropyl 2,2′-((((2- (((3S,6S,7aS, 8aR,9aR)-3-(3- (morpholine-4- carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) phosphoryl) bis(azanediyl)) (2S,2′S)- dipropionate | 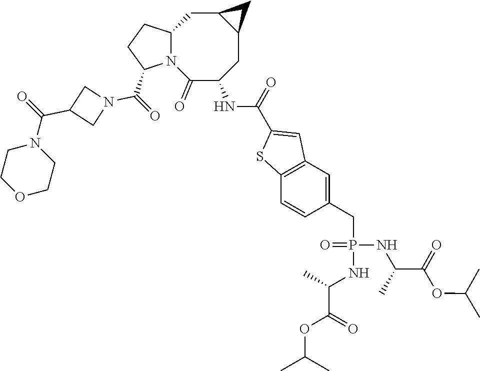 [SMILES: CC(C)OC(=O)[C@H](C)NP(=O)(Cc1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1)N[C@@H](C)C(=O)OC(C)C] | 871.3 [M + H]⁺ | ¹H NMR (400 MHz, CDCl₃) δ 7.79 (m, 3H), 7.40 (m, 2H), 5.00 (m, 3H), 4.77 (m, 1H), 4.59-4.04 (m, 5H), 3.94 (m, 2H), 3.67 (m, 7H), 3.29 (m, 4H), 2.29 (m, 3H), 2.15-2.01 (m, 4H), 1.72-1.63 (m, 1H), 1.44 (m, 1H), 1.32 (m, 6H), 1.25-1.10 (m, 12H), 0.95 (m 1H), 0.82 (m, 1H), 0.01 (m, 1H). |
| 148 | diisopropyl (((((2- (((3S,6S,7aS, 8aR,9aR)-3-(3- (morpholine-4- carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) phosphoryl bis(oxy)) bis(methylene)) bis(carbonate) | 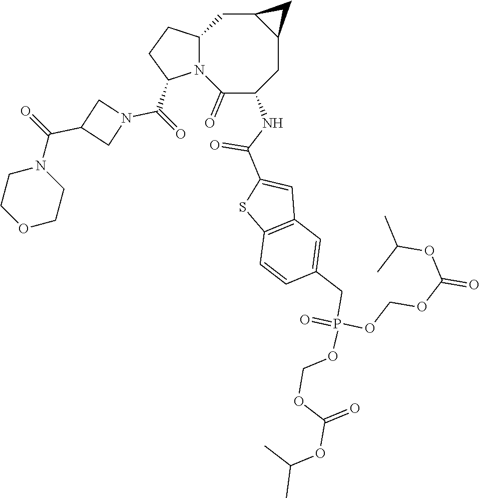 [SMILES: CC(C)OC(=O)OCOP(=O)(Cc1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1)OCOC(=O)OC(C)C] | 877.3 [M + H]⁺ | ¹H NMR (400 MHz, CDCl₃) δ 7.83- 7.72 (m, 3H), 7.43- 7.30 (m, 2H), 5.66- 5.56 (m, 4H), 5.09- 4.73 (m, 4H), 4.57- 4.04 (m, 5H), 3.73- 3.60 (m, 6H), 3.44- 3.22 (m, 4H), 2.37- 2.25 (m, 3H), 2.16- 1.99 (m, 4H), 1.82- 1.85 (m, 2H) 1.74- 1.63 (m, 1H), 1.51- 1.38 (m, 1H), 1.30 (dd, J = 6.2, 1.6 Hz, 12H), 1.00-0.77 (m, 2H). |
| 149 | S,S′-(((((2- (((3S,6S,7aS, 8aR,9aR)-3-(3- (morpholine-4- carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) phosphoryl) bis(oxy)) bis(ethane-2,1- diyl)) dibutanethioate | 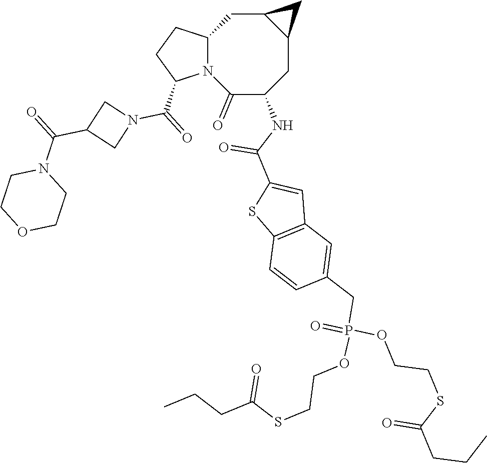 [SMILES: CCCC(=O)SCCOP(=O)(Cc1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1)OCCSC(=O)CCC] | 905.3 [M + H]⁺ | ¹H NMR (400 MHz, CDCl₃) δ 7.86-7.68 (m, 3H), 7.52-7.35 (m, 2H), 5.05-4.90 (m, 1H), 4.77 (t, J = 8.4 Hz, 1H), 4.55-4.52 (m, 1H), 4.45-4.40 (m, 1H), 4.31 (t, J = 9.1 Hz, 1H), 4.21-4.14 (m, 1H), 4.10-4.03 (m, 5H), 3.73-3.58 (m, 7H), 3.32-3.27 (m, 4H), 3.07 (t, J = 6.0 Hz, 4H), 2.52 (t, J = 7.4 Hz, 4H), 2.31-2.28 (m, 3H), 2.12-2.03 (m, 4H), 1.72-1.63 (m, 6H), 1.44 (s, 1H), 1.24 (t, J = 7.0 Hz, 1H), 0.94 (t, J = 7.4 Hz, 6H), 0.88-0.78 (m, 1H). |
| 150 | ((((2-(((3S,6S, 7aS,8aR,9aR)-3- (3-morpholine-4- carbonyl) azetidine-1- carbonyl)-5- oxodecahydro- 1H-cyclopropa [d]pyrrolo[1,2- a]azocin-6-yl) carbamoyl)benzo [b]thiophen-5-yl) methyl) phosphoryl) bis(oxy)) bis(methylene) bis(2,2- dimethyl- | 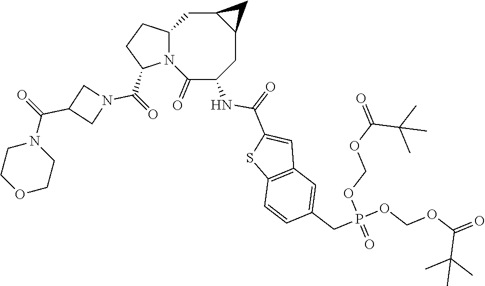 [SMILES: CC(C)(C)C(=O)OCOP(=O)(Cc1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1)OCOC(=O)C(C)(C)C] | 873.3 [M + H]⁺ | ¹H NMR (400 MHz, CDCl₃) δ 7.82-7.70 (m, 3H), 7.52-7.30 (m, 2H), 5.67-5.52 (m, 4H), 5.11-4.99 (m, 1H), 4.97-4.71 (m, 1H), 4.60-4.47 (m, 1H), 4.47-4.37 (m, 1H), 4.35-4.25 (m, 1H), 4.23-4.12 (m, 1H), 4.12-4.03 (m, 1H), 3.75-3.51 (m, 7H), 3.35 (d, J = 22.1 Hz, 2H), 3.30- 3.20 (m, 2H), 2.40- 2.22 (m, 3H), 2.16- 2.08 (m, 2H), 2.05- 1.98 (m, 2H), 1.76- |
| | propanoate) | | | 1.60 (m, 1H), 1.59- |
| | | | | 1.36 (m, 2H), 1.20 (s, |
| | | | | 18H), 1.00-0.87 (m, |
| | | | | 1H), 0.88-0.76 (m, 1H). |
| 151 | S,S′-(((((2- (((3S,6S,7aS, 8aR,9aR)-3-(3- (1H-imidazol-1- yl)azetidine-1- carbonyl)-5- oxodecahydro- 1H-cyclopropa [d]pyrrolo[1,2- a]azocin-6- yl)carbamoyl) benzo[b] thiophen-5- yl)methyl) phosphoryl) bis(oxy)) bis(ethane-2,1- diyl)) bis(2,2- dimethyl- propanethioate) | 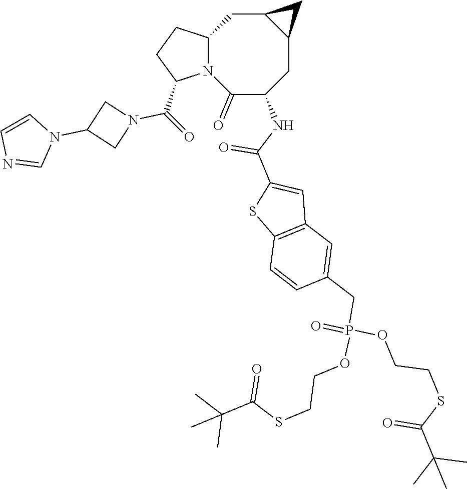 [SMILES: CC(C)(C)C(=O)SCCOP(=O)(Cc1ccc2sc(C(=O)N[C@H]3C[C@@H]4C[C@@H]4C[C@H]4CC[C@@H](C(=O)N5CC(n6ccnc6)C5)N4C3=O)cc2c1)OCCSC(=O)C(C)(C)C] | 886.3 [M + H]⁺ | ¹H NMR (400 MHz, CDCl₃) δ 8.06-7.69 (m, 4H), 7.51-7.37 (m, 2H), 7.30-7.28 (m, 1H), 7.22-7.18 (m, 1H), 5.08-5.03 (m, 2H), 4.74-4.67 (m, 1H), 4.45-4.13 (m, 4H), 4.05-4.02 (m, 4H), 3.30 (d, J = 21.6 Hz, 2H), 3.06- 3.02 (m, 4H), 2.44- 2.01 (m, 7H), 1.74- 1.65 (m, 2H), 1.45- 0.82 (m, 22H). |
| 152 | ((((R)- fluoro(2- (((3S,6S,7aR, 8aS,9aR)-5- oxo-3-(3- (pyridin-3- yl)azetidine- 1- carbonyl) decahydro-1H- cyclopropa[d] pyrrolo[1,2-a] azocin-6- yl)carbamoyl) benzo[b] thiophen-5- yl)methyl) phosphoryl) bis(oxy)) bis(methylene) diisopropyl bis(carbonate) | 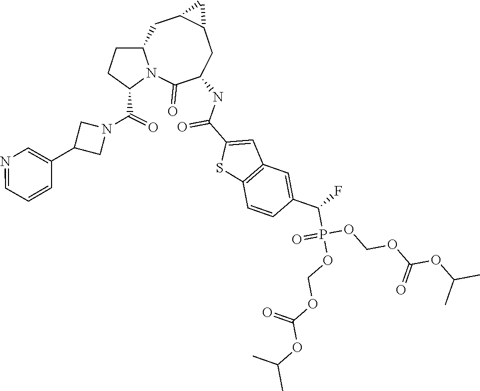 | 859.0 [M + H]⁺ | ¹H NMR (400 MHz, CDCl₃) δ 9.05-8.77 (m, 1H), 8.78-8.62 (m, 1H), 8.53-8.06 (m, 1H), 8.03-7.65 (m, 5H), 7.53 (d, J = 8.2 Hz, 1H), 5.99- 5.81 (m, 1H), 5.69- 5.63 (m, 4H), 5.16- 4.95 (m, 1H), 4.95- 4.62 (m, 4H), 4.51- 3.87 (m, 5H), 2.45- 2.28 (m, 1H), 2.26- 2.09 (m, 3H), 2.01- 1.94 (m, 1H), 1.83- 1.55 (m, 2H), 1.51- 1.38 (m, 1H), 1.31 (d, J = 6.2 Hz, 12H), 1.28- 1.11 (m, 2H), 0.87- 0.78 (m, 1H), 0.26- 0.18 (m, 1H). |
| 153 | isopropyl ((fluoro(2- (((3S,6S,7aS, 8aR,9aR)-3- (3- (morpholine-4- carbonyl) azetidine-1- carbonyl)-5- oxodecahydro- 1H- cyclopropa[d] pyrrolo[1,2-a] azocin-6- yl)carbamoyl) benzo[b] thiophen-5- yl)methyl) phenoxy) phosphoryl)-L- alaninate | 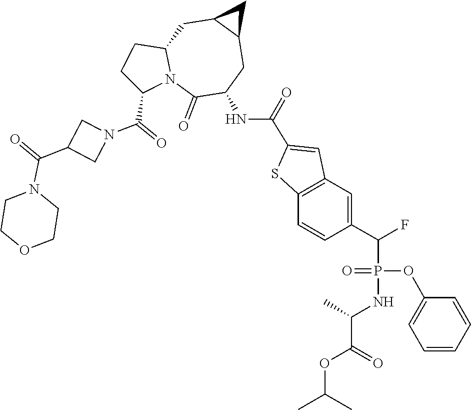 [SMILES: CC(C)OC(=O)[C@H](C)NP(=O)(Oc1ccccc1)C(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 852.3 [M + H]⁺ | ¹H NMR (400 MHz, DMSO-d₆) δ 9.02- 8.74 (m, 1H), 8.32 (s, 1H), 8.14-8.01 (m, 2H), 7.69-7.54 (m, 1H), 7.43-7.27 (m, 2H), 7.25-7.08 (m, 3H), 6.32-6.01 (m, 2H), 4.99-4.69 (m, 2H), 4.68-4.47 (m, 1H), 4.38-4.25 (m, 2H), 4.24-4.09 (m, 1H), 4.06-3.85 (m, 2H), 3.82-3.61 (m, 2H), 3.58-3.41 (m, 6H), 3.27-3.13 (m, 2H), 2.40-2.24 (m, 1H), 2.17-1.93 (m, 4H), 1.91-1.73 (m, 2H), 1.69-1.56 (m, 1H), 1.46-1.27 (m, 1H), 1.18-0.90 (m, 9H), 0.86-0.65 (m, 2H), 0.04-−0.06 (m, 1H). |
| 154 | isopropyl (((2- (((3S,6S,7aS, 8aR,9aR)-3-(3- (morpholine-4- carbonyl) azetidine-1- carbonyl)-5- oxodecahydro- 1H- cyclopropa[d] pyrrolo[1,2-a] azocin-6- yl)carbamoyl) benzo[b] thiophen-5- yl)methyl) (phenoxy) (phosphoryl)-L- alaninate | 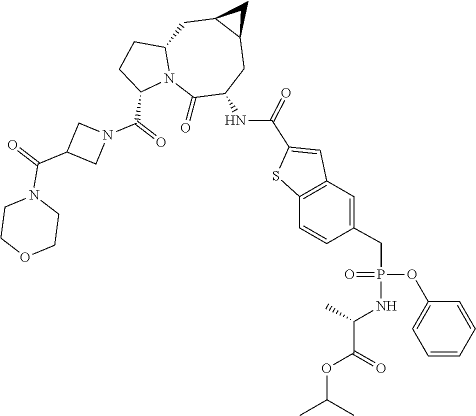 [SMILES: CC(C)OC(=O)[C@H](C)NP(=O)(Cc1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1)Oc1ccccc1] | 834.2 [M + H]⁺ | ¹H NMR (400 MHz, DMSO-d₆) δ 8.90- 8.75 (m, 1H), 8.23 (s, 1H), 7.98-7.85 (m, 2H), 7.44 (d, J = 8.4 Hz, 1H), 7.36-7.29 (m, 2H), 7.18-7.09 (m, 3H), 4.98-4.88 (m, 1H), 4.85-4.74 (m, 1H), 4.66-4.48 (m, 1H), 4.38-4.26 (m, 2H), 4.20-4.12 (m, 1H), 4.06-3.61 (m, 4H), 3.58-3.39 (m, 8H), 3.27-3.20 (m, 2H), 2.36-2.27 (m, 1H), 2.17-1.95 (m, 4H), 1.90-1.75 (m, 2H), 1.69-1.54 (m, 1H), 1.34 (s, 1H), 1.13-0.96 (m, 9H), 0.83-0.67 (m, 2H), 0.02-−0.04 (m, 1H). |
| 155 | ((((2- (((3S,6S,7aR, 8aS,9aR)-5- oxo-3-(3- (pyridin-2- yl)azetidine-1- carbonyl) decahydro-1H- cyclopropa[d] pyrrolo[1,2- a]azocin-6- yl)carbamoyl) benzo[b] thiophen-5- yl)methyl) phosphoryl) bis(oxy)) bis(methylene) bis(2,2- dimethyl- pronanoate) | 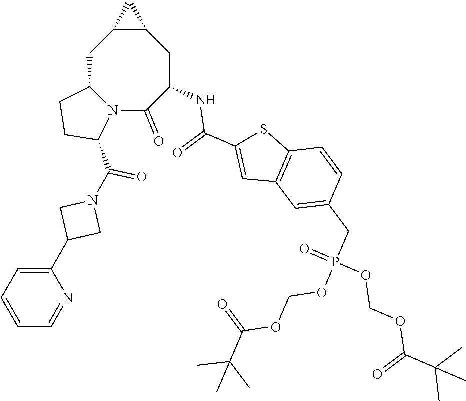 [SMILES: CC(C)(C)C(=O)OCOP(=O)(Cc1ccc2sc(C(=O)N[C@H]3C[C@H]4C[C@H]4C[C@H]4CC[C@@H](C(=O)N5CC(c6ccccn6)C5)N4C3=O)cc2c1)OCOC(=O)C(C)(C)C] | 837.0 [M + H]⁺ | ¹H NMR (400 MHz, DMSO-d₆) δ 8.85- 8.76 (m, 1H), 8.63- 8.51 (m, 1H), 8.25- 8.14 (m, 1H), 8.01- 7.91 (m, 1H), 7.86- 7.69 (m, 2H), 7.40- 7.21 (m, 3H), 5.64- 5.51 (m, 4H), 4.87- 4.78 (m, 1H), 4.77- 4.65 (m, 1H), 4.60- 4.41 (m, 1H), 4.37- 4.26 (m, 1H), 4.26- 4.11 (m, 2H), 4.08- 3.92 (m, 2H), 3.62- 3.43 (m, 2H), 2.28- 1.81 (m, 6H), 1.74- 1.47 (m, 2H), 1.39- 1.19 (m, 2H), 1.15- 1.07 (m, 18H), 0.81- 0.72 (m, 1H), 0.09- 0.02 (m, 1H). |
| 156 | ((((2- (((3S,6S,7aS, 8aR,9aR)-3- (3-(1H- imidazol-1- yl)azetidine-1- carbonyl)-5- oxodecahydro- 1H- cyclopropa[d] pyrrolo[1,2- a]azocin-6- yl)carbamoyl) benzo[b] thiophen-5- yl)methyl) phosphoryl) bis(oxy)) bis(methylene) bis(2,2- dimethyl- propanoate) | 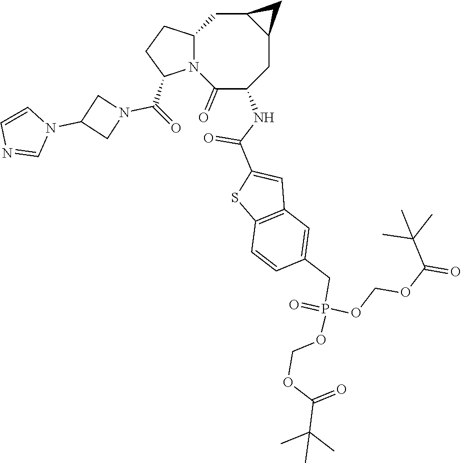 [SMILES: CC(C)(C)C(=O)OCOP(=O)(Cc1ccc2sc(C(=O)N[C@H]3C[C@@H]4C[C@@H]4C[C@H]4CC[C@@H](C(=O)N5CC(n6ccnc6)C5)N4C3=O)cc2c1)OCOC(=O)C(C)(C)C] | 826.3 [M + H]⁺ | ¹H NMR (400 MHz, CDCl₃) δ 8.75 (s, 1H), 8.12-7.84 (m, 2H), 7.76-7.61 (m, 2H), 7.30-7.20 (m, 2H), 7.15-7.05 (m, 1H), 5.62-5.43 (m, 4H), 5.19-5.07 (m, 1H), 5.04-4.76 (m, 2H), 4.66-4.46 (m, 1H), 4.35-3.87 (m, 4H), 3.33-3.20 (m, 2H), 2.58-2.18 (m, 3H), 2.12-1.87 (m, 4H), 1.47-1.43 (m, 1H), 1.31-1.22 (m, 2H), 1.14-1.09 (m, 18H), 0.93-0.84 (m, 1H), −0.07-−0.08 (m, 1H). |
| 157 | ((((2- (((3S,6S,7aS, 8aR,9aR)-3- (3-(1H- imidazol-1- yl)azetidine-1- carbonyl)-5- oxodecahydro- 1H- cyclopropa[d] pyrrolo[1,2- a]azocin-6- yl)carbamoyl) benzo[b] thiophen-5- yl)methyl) phosphoryl) bis(oxy)) bis(methylene) diisopropyl bis(carbonate) | 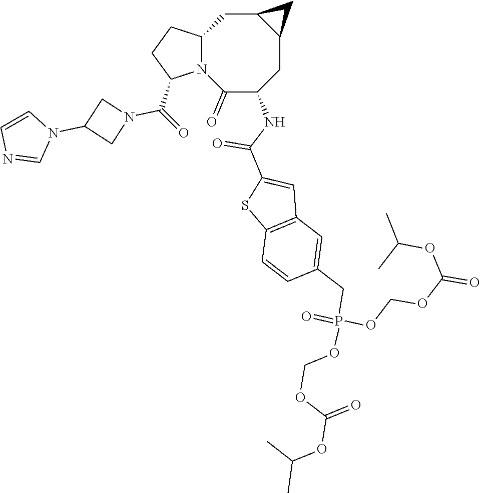 [SMILES: CC(C)OC(=O)OCOP(=O)(Cc1ccc2sc(C(=O)N[C@H]3C[C@@H]4C[C@@H]4C[C@H]4CC[C@@H](C(=O)N5CC(n6ccnc6)C5)N4C3=O)cc2c1)OCOC(=O)OC(C)C] | 829.9 [M + H]⁺ | ¹H NMR (400 MHz, CDCl₃) δ 7.94-7.67 (m, 4H), 7.51-7.30 (m, 3H), 7.20-7.17 (m, 1H), 5.63-5.57 (m, 4H), 5.30-5.05 (m, 3H), 4.99-4.87 (m, 2H), 4.77-4.64 (m, 1H), 4.47-4.14 (m, 4H), 3.39 (d, J = 22.1 Hz, 2H), 2.40-2.31 (m, 3H), 2.20-2.00 (m, 4H), 1.75-1.69 (m, 1H), 1.58-1.38 (m, 2H), 1.30 (d, J = 5.9 Hz, 12H), 1.03-0.78 (m, 2H). |
| 158 | S,S′-(((((2- (((3S,6S,7aS, 8aR,9aR)-3- (3-(1H- imidazol-1- yl)azetidine- 1-carbonyl)-5- oxodecahydro- 1H- cyclopropa[d] pyrrolo[1,2- a]azocin-6- yl)carbamoyl) benzo[b] thiophen-5- yl)methyl) phosphoryl) bis(oxy)) bis(ethane-2,1- diyl)) dibutanethioate | 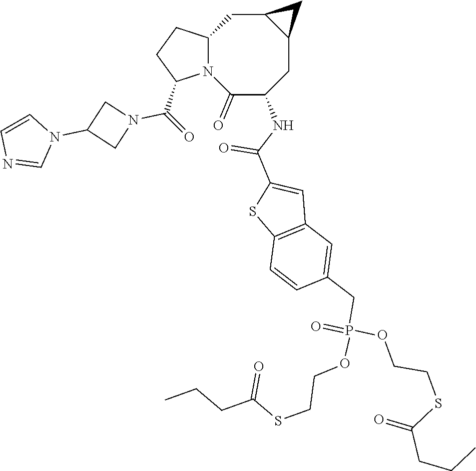 [SMILES: CCCC(=O)SCCOP(=O)(Cc1ccc2sc(C(=O)N[C@H]3C[C@@H]4C[C@@H]4C[C@H]4CC[C@@H](C(=O)N5CC(n6ccnc6)C5)N4C3=O)cc2c1)OCCSC(=O)CCC] | 858 [M + H]⁺ | ¹H NMR (400 MHz, DMSO-d₆) δ 8.93- 8.79 (m, 1H), 8.26- 8.18 (m, 1H), 7.97- 7.90 (m, 1H), 7.88- 7.79 (m 2H), 7.50- 7.32 (m, 2H), 7.02- 6.91 (m, 1H), 5.21- 5.10 (m, 1H), 4.97- 4.90 (m, 1H), 4.74- 4.60 (m, 1H), 4.41- 4.31 (m, 2H) 4.21- 4.13 (m, 1H), 4.00- 3.96 (m, 4H), 3.45- 3.38 (m, 4H), 3.06 (t, J = 6.2 Hz, 4H), 2.56- 2.51 (m, 4H), 2.37- 2.30 (m, 1H), 2.18- 1.95 (m, 5H), 1.90- 1.78 (m, 2H), 1.69- 1.61 (m, 1H), 1.58- 1.51 (m, 4H), 1.37- 1.28 (m, 1H), 0.85 (t, J = 7.3 Hz, 6H), 0.73- 0.67 (m, 1H), 0.02- −0.03 (m, 1H) |
| 159 | benzyl (2S)-2- (((difluoro(2- (((3S,6S,7aS, 8aR,9aR)-3-(3- (morpholine-4- carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) (phenoxy) phosphoryl) amino)-4- phenylbutanoate | 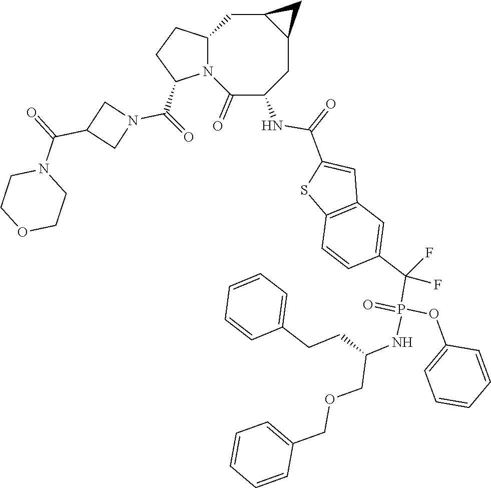 [SMILES: O=C(N[C@H]1C[C@@H]2C[C@@H]2C[C@H]2CC[C@@H](C(=O)N3CC(C(=O)N4CCOCC4)C3)N2C1=O)c1cc2cc(C(F)(F)P(=O)(N[C@@H](CCc3ccccc3)COCc3ccccc3)Oc3ccccc3)ccc2s1] | 1008.1 [M + H]⁺ | ¹H NMR (400 MHz, CD₃OD) δ 8.20 (s, 1H), 8.18-8.10 (m, 1H), 8.06-7.88 (m, 1H), 7.75-7.65 (m, 1H), 7.37-7.26 (m, 7H), 7.23-7.09 (m, 6H), 6.97-6.85 (m, 2H), 5.13-4.91 (m, 3H), 4.77-4.65 (m, 1H), 4.53-4.36 (m, 2H), 4.33-4.15 (m, 2H), 4.12-4.02 (m, 1H), 3.97-3.72 (m, 2H), 3.69-3.56 (m, 5H), 3.55-3.45 (m, 1H), 3.37-3.34 (m, 1H), 2.53-2.38 (m, 2H), 2.37-2.06 (m, 5H), 2.04-1.84 (m, 3H), 1.83-1.65 (m, 2H), 1.47-1.24 (m, 2H), 1.01-0.89 (m, 1H), 0.88-0.79 (m, 1H), 0.15-−0.04 (m, 1H). |
| 160 | diethyl 2,2′- ((((((((2-(((3S, 6S,7aS,8aR, 9aR)-3-(3- (morpholine-4- carbonyl) azetidine-1- carbonyl)-5- oxodecahydro- 1H-cyclopropa [d]pyrrolo[1,2- a]azocin-6- yl)carbamoyl) benzo[b] thiophen-5- yl)methyl) phosphoryl) bis(oxy)) bis(methylene)) bis(oxy)) bis(carbonate)) bis(azanediyl)) (2S,2′S)- dipropionate | 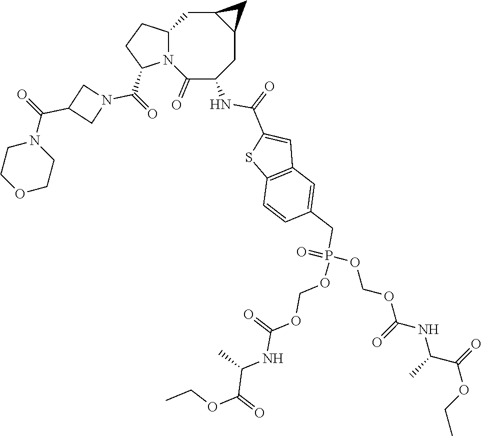 [SMILES: CCOC(=O)[C@H](C)NC(=O)OCOP(=O)(Cc1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1)OCOC(=O)N[C@@H](C)C(=O)OCC] | 991.2 [M + H]⁺ | ¹H NMR (400 MHz, CD₃CN) δ 7.91-7.74 (m, 4H), 7.34 (d, J = 8.3 Hz, 1H), 6.36-6.23 (m, 2H), 5.60-5.47 (m, 4H), 5.02-4.92 (m, 1H), 4.65-4.56 (m, 1H), 4.41-4.28 (m, 2H), 4.23-3.91 (m, 9H), 3.65-3.44 (m, 7H), 3.38 (d, J = 21.8 Hz, 2H), 3.26-3.17 (m, 2H), 2.36-2.26 (m, 1H), 2.24-2.18 (m, 2H), 2.11-2.02 (m, 3H), 1.90-1.84 (m, 1H), 1.69-1.58 (m, 1H), 1.46-1.38 (m, 1H), 1.37-1.32 (m, 6H), 1.25-1.18 (m, 6H), 0.85-0.72 (m, 2H), 0.03-−0.03 (m, 1H). |
| 161 | (((difluoro(2- (((3S,6S,7aS, 8aR,9aR)-3-(3- (morpholine-4- carbonyl) azetidine-1- carbonyl)-5- oxodecahydro- 1H-cyclopropa [d]pyrrolo[1,2- a]azocin-6- yl)carbamoyl) benzo[b] thiophen-5- yl)methyl) phosphoryl) bis(oxy)) bis(methylene) bis(1-methoxy- 2-methylpropan- 2-yl) bis(carbonate) | 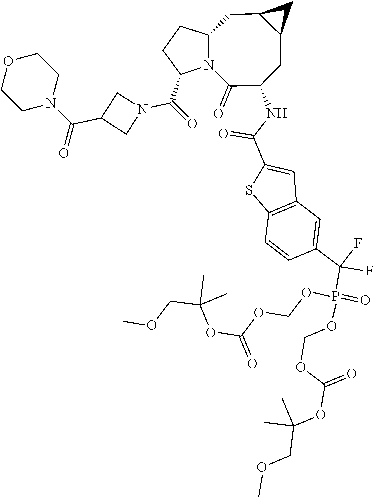 [SMILES: COCC(C)(C)OC(=O)OCOP(=O)(OCOC(=O)OC(C)(C)COC)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 1001.0 [M + H]⁺ | ¹H NMR (400 MHz, CD₃CN) δ 7.92 (s, 1H), 7.85 (d, J = 8.3 Hz, 1H), 7.82-7.78 (m, 1H), 7.63-7.54 (m, 1H), 7.39 (d, J = 8.6 Hz, 1H), 5.50-5.32 (m, 4H), 4.81-4.71 (m, 1H), 4.44-4.34 (m, 1H), 4.22-4.08 (m, 2H), 4.04-3.95 (m, 1H), 3.92-3.71 (m, 2H), 3.45-3.30 (m, 7H), 3.28 (s, 4H), 3.11 (s, 6H), 3.06-2.99 (m, 2H), 2.17-2.06 (m, 1H), 2.04-1.98 (m, 1H), 1.91-1.85 (m, 3H), 1.71-1.66 (m, 1H), 1.49-1.37 (m, 1H), 1.23 (s, 12H), 1.10-1.05 (m, 2H), 0.67-0.52 (m, 2H), −0.16-−0.22 (m, 1H). |
| 162 | (((difluoro(2- (((3S,6S,7aS, 8aR,9aR)-3-(3- (morpholine-4- carbonyl) azetidine-1- carbonyl)-5- oxodecahydro- 1H-cyclopropa [d]pyrrolo[1,2- a]azocin-6- yl)carbamoyl) benzo[b] thiophen-5- yl)methyl) phosphoryl) bis(oxy)) bis(methylene) bis(tetrahydro- 2H-pyran-4- yl) bis(carbonate) | 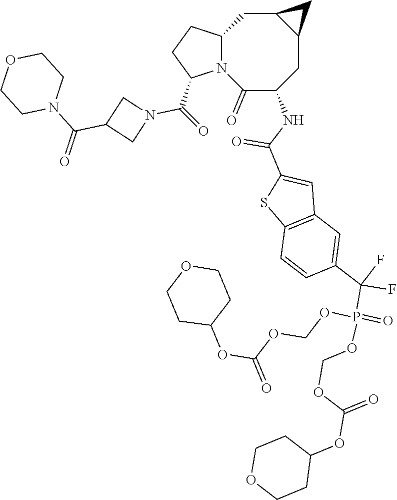 [SMILES: O=C(OCOP(=O)(OCOC(=O)OC1CCOCC1)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1)OC1CCOCC1] | 997.1 [M + H]⁺ | ¹H NMR (400 MHz, CD₃CN) δ 8.11 (s, 1H), 8.06-8.01 (m, 1H), 8.00 (d, J = 4.6 Hz, 1H), 7.83 (dd, J = 15.0, 6.7 Hz, 1H), 7.59 (d, J = 8.6 Hz, 1H), 5.72- 5.61 (m, 4H), 5.00- 4.93 (m, 1H), 4.77 (tt, J = 8.7, 4.2 Hz, 2H), 4.64-4.56 (m, 1H), 4.40-4.29 (m, 2H), 4.23-4.16 (m, 1H), 4.12-3.90 (m, 3H), 3.86-3.77 (m, 4H), 3.66-3.41 (m, 11H), 3.27-3.20 (m, 2H), 2.37-2.27 (m, 1H), 2.25-2.17 (m, 1H), 2.12-2.04 (m, 4H), 1.91-1.86 (m, 4H), 1.69-1.55 (m, 5H), 1.48-1.36 (m, 1H), 0.86-0.74 (m, 2H), 0.04-−0.02 (m, 1H). |
| 163 | benzyl ((difluoro(2- (((3S,6S,7aS, 8aR,9aR)-3-(3- (morpholine-4- carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) (phenoxy) phosphoryl)-L- leucinate | 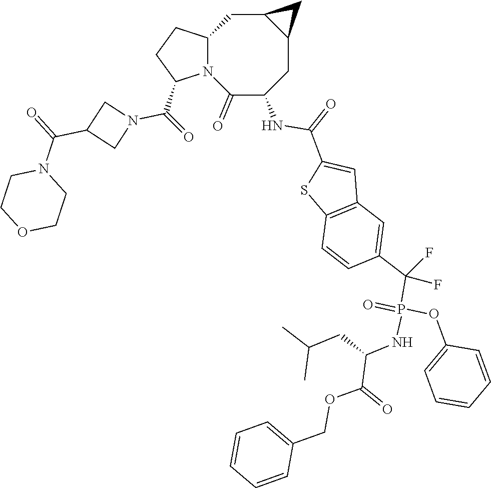 [SMILES: CC(C)C[C@H](NP(=O)(Oc1ccccc1)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1)C(=O)OCc1ccccc1] | 960.1 [M + H]⁺ | ¹H NMR (400 MHz, CD₃OD) δ 8.24-8.07 (m, 2H), 8.05-7.96 (m, 1H), 7.74-7.66 (m, 1H), 7.37-7.21 (m, 7H), 7.20-7.04 (m, 3H), 5.09-4.89 (m, 3H), 4.76-4.67 (m, 1H), 4.52-4.37 (m, 2H), 4.33-4.16 (m, 2H), 4.11-4.01 (m, 1H), 3.95-3.85 (m, 1H), 3.82-3.71 (m, 1H), 3.68-3.47 (m, 6H), 3.38-3.32 (m, 2H), 2.48-2.38 (m, 1H), 2.34-2.07 (m, 4H), 2.04-1.91 (m, 2H), 1.78-1.66 (m, 1H), 1.53-1.27 (m, 4H), 0.98-0.88 (m, 1H), 0.87-0.79 (m, 1H), 0.78-0.65 (m, 6H), 0.09-0.02 (m, 1H). |
| 164 | benzyl ((difluoro(2- (((3S,6S,7aS, 8aR,9aR)-3-(3- (morpholine-4- carbonyl) azetidine-1- carbonyl)-5- oxodecahydro- 1H-cyclopropa [d]pyrrolo[1,2- a]azocin-6- yl)carbamoyl) benzo[b] thiophen-5- yl)methyl)(2- ((3-methyl- butanoyl)thio) ethoxy) phosphoryl)-L- alaninate | 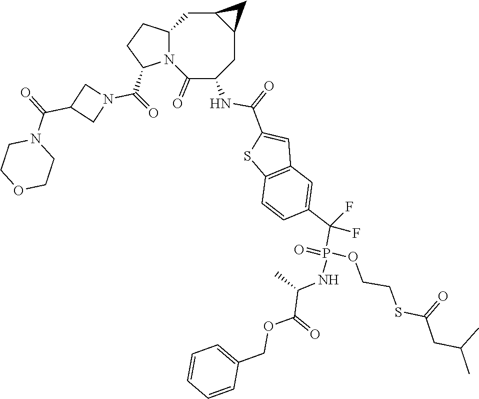 [SMILES: CC(C)CC(=O)SCCOP(=O)(N[C@@H](C)C(=O)OCc1ccccc1)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 986.1 [M + H]⁺ | ¹H NMR (400 MHz, CD₃OD) δ 8.19-8.14 (m, 1H), 8.14-8.09 (m, 1H), 8.06-7.93 (m, 1H), 7.66-7.59 (m, 1H), 7.40-7.28 (m, 5H), 5.21-5.07 (m, 2H), 5.06-4.99 (m, 1H), 4.77-4.67 (m, 1H), 4.53-4.38 (m, 2H), 4.34-4.02 (m, 5H), 3.97-3.87 (m, 1H), 3.83-3.71 (m, 1H), 3.68-3.50 (m, 6H), 3.40-3.35 (m, 1H), 3.11-3.03 (m, 2H), 2.53-1.89 (m, 11H), 1.79-1.64 (m, 1H), 1.46-1.32 (m, 4H), 0.99-0.93 (m, 1H), 0.91 (d, J = 6.6 Hz, 6H), 0.88-0.80 (m, 1H), 0.11-0.01 (m, 1H). |
| 165 | isopropyl ((difluoro(2- (((3S,6S,7aS, 8aR,9aR)-3-(3- (morpholine-4- carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) (phenoxy) phosphoryl)-L- phenylalaninate | 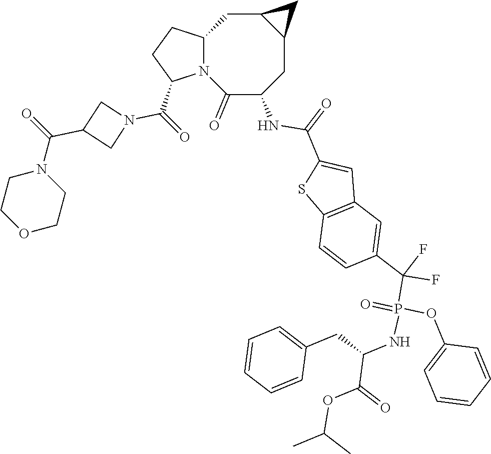 [SMILES: CC(C)OC(=O)[C@H](Cc1ccccc1)NP(=O)(Oc1ccccc1)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 946.2 [M + H]⁺ | ¹H NMR (400 MHz, CD₃OD) δ 8.25-7.95 (m, 3H), 7.77-7.57 (m, 1H), 7.39-7.12 (m, 6H), 7.12-6.89 (m, 4H), 5.07-4.99 (m, 1H), 4.84-4.67 (m, 2H), 4.60-4.37 (m, 2H), 4.33-4.11 (m, 2H), 4.10-3.96 (m, 1H), 3.82-3.70 (m, 1H), 3.68-3.46 (m, 6H), 3.36 (br. s., 1H), 3.01-2.72 (m, 1H), 2.50-2.38 (m, 1H), 2.34-2.07 (m, 4H), 2.03-1.91 (m, 2H), 1.80-1.65 (m, 1H), 1.47-1.31 (m, 2H), 1.29 (s, 4H), 1.14-1.05 (m, 2H), 1.01-0.88 (m, 2H), 0.87-0.81 (m, 1H), 0.10-0.02 (m, 2H). |
| 166 | (((difluoro(2- (((3S,6S,7aR, 8aS,9aR)-5-oxo- 3-(3-(pyridin-3- yl)azetidine-1- carbonyl) decahydro-1H- cyclopropa[d] pyrrolo[1,2- a]azocin-6- yl)carbamoyl) benzo[b] thiophen-5- yl)methyl) phosphoryl) bis(oxy)) bis(methylene) bis(tetrahydro- 2H-pyran-4-yl) bis(carbonate) | 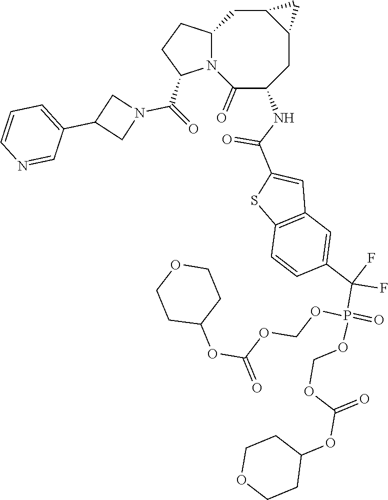 [SMILES: O=C(OCOP(=O)(OCOC(=O)OC1CCOCC1)C(F)(F)c1ccc2sc(C(=O)N[C@H]3C[C@H]4C[C@H]4C[C@H]4CC[C@@H](C(=O)N5CC(c6cccnc6)C5)N4C3=O)cc2c1)OC1CCOCC1] | 961.1 [M + H]⁺ | ¹H NMR (400 MHz, CD₃CN) δ 8.57 (d, J = 14.2 Hz, 1H), 8.48 (d, J = 4.2 Hz, 1H), 8.17- 8.11 (m, 1H), 8.10- 7.97 (m, 2.5H), 7.96- 7.88 (m, 1H), 7.82- 7.75 (m, 0.5H), 7.60 (d, J = 8.6 Hz, 1H), 7.42- 7.32 (m, 1H), 5.74- 5.60 (m, 4H), 4.91- 4.71 (m, 3.5H), 4.62 (t, J = 8.4 Hz, 0.5H), 4.51- 4.34 (m, 2H), 4.33- 4.26 (m, 0.5H), 4.25- 4.15 (m, 1.5H), 4.00- 3.86 (m, 2H), 3.86- 3.75 (m, 4H), 3.51- 3.39 (m, 4H), 2.29- 2.21 (m, 2H), 2.13- 2.02 (m, 3H), 1.91- 1.85 (m, 4H), 1.84- 1.74 (m, 2H), 1.69- 1.55 (m, 5H), 1.54- 1.43 (m, 1H), 1.33- 1.22 (m, 1H), 0.83- 0.75 (m, 1H), 0.16- 0.07 (m, 1H). |
| 167 | isopropyl (2S)-2- (((difluoro(2- (((3S,6S,7aS, 8aR,9aR)-3-(3- (morpholine-4- carbonyl) azetidine-1- carbonyl)-5- oxodecahydro- 1H- cyclopropa[d] pyrrolo[1,2-a] azocin-6- yl)carbamoyl) benzo[b] thiophen-5- yl)methyl) (phenoxy) phosphoryl) oxy)-3-phenyl- propanoate | 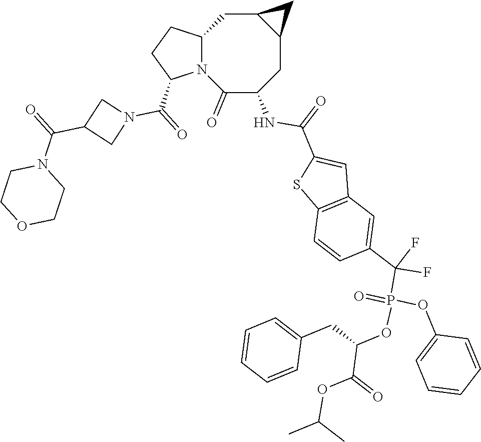 [SMILES: CC(C)OC(=O)[C@H](Cc1ccccc1)OP(=O)(Oc1ccccc1)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 947.1 [M + H]⁺ | 1H NMR (400 MHz, CD₃OD) δ 8.15-7.92 (m, 3H), 7.64-7.44 (m, 1H), 7.36-7.10 (m, 8H), 7.07-7.03 (m, 1H), 6.96-6.92 (m, 1H), 5.21-4.89 (m 3H), 4.76-4.67 (m, 1H), 4.54-4.37 (m, 2H), 4.33-4.16 (m, 2H), 4.10-4.03 (m, 1H), 3.80-3.69 (m, 1H), 3.67-3.41 (m, 6H), 3.36-3.32 (m, 1H), 3.29-3.03 (m, 3H), 2.51-2.39 (m, 1H), 2.34-2.06 (m, 4H), 2.04-1.91 (m, 2H), 1.79-1.67 (m, 1H), 1.47-1.35 (m, 1H), 1.16-1.05 (m, 6H), 0.99-0.79 (m, 2H), 0.09-0.02 (m, 1H). |
| 168 | (((difluoro(2- (((3S,6S,7aR, 8aS,9aR)-5- oxo-3-(3- (pyridin-3- yl)azetidine-1- carbonyl) decahydro-1H- cyclopropa[d] pyrrolo[1,2- a]azocin-6- yl)carbamoyl) benzo[b] thiophen-5- yl)methyl) phosphoryl) bis(oxy)) bis(methylene) bis(1-methoxy- 2-methylpropan- 2-yl) bis(carbonate) | 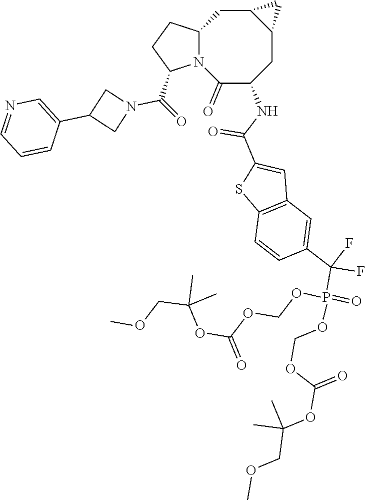 [SMILES: COCC(C)(C)OC(=O)OCOP(=O)(OCOC(=O)OC(C)(C)COC)C(F)(F)c1ccc2sc(C(=O)N[C@H]3C[C@H]4C[C@H]4C[C@H]4CC[C@@H](C(=O)N5CC(c6cccnc6)C5)N4C3=O)cc2c1] | 965.2 [M + H]⁺ | ¹H NMR (400 MHz, CD₃CN) δ 8.61-8.53 (m, 1H), 8.51-8.43 (m, 1H), 8.18-8.11 (m, 1H), 8.11-8.05 (m, 1H), 8.02 (s, 1H), 7.99- 7.75 (m, 2H), 7.61 (d, J = 8.3 Hz, 1H), 7.44- 7.30 (m, 1H), 5.69- 5.54 (m, 4H), 4.91- 4.83 (m, 1H), 4.82- 4.57 (m, 1H), 4.50- 4.33 (m, 2H), 4.33- 4.16 (m, 2H), 3.99- 3.84 (m, 2H), 3.52- 3.43 (m, 4H), 3.35- 3.27 (m, 6H), 2.30- 2.19 (m, 2H), 2.13- 2.00 (m, 3H), 1.84- 1.74 (m, 1H), 1.70- 1.57 (m, 1H), 1.55- 1.46 (m, 1H), 1.45- 1.41 (m, 12H), 1.32- 1.22 (m, 2H), 0.83- 0.75 (m, 1H), 0.15- 0.09 (m, 1H). |
| 169 | (((difluoro(2- (((3S,6S,7aS, 8aR,9aR)-3-(3- (morpholine-4- carbonyl) azetidine-1- carbonyl)-5- oxodecahydro- 1H-cyclopropa [d]pyrrolo[1,2- a]azocin-6-yl) carbamoyl) benzo[b] thiophen-5- yl)methyl) phosphoryl) bis(oxy)) bis(methylene) bis(3- methoxypropyl) bis(carbonate) | 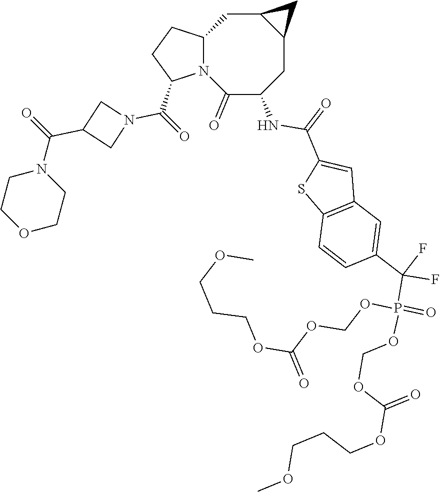 [SMILES: COCCCOC(=O)OCOP(=O)(OCOC(=O)OCCCOC)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 973.4 [M + H]⁺ | ¹H NMR (400 MHz, CD₃CN) δ 8.12 (s, 1H), 8.05 (d, J = 8.8 Hz, 1H), 8.02-7.99 (m, 1H), 7.79-7.71 (m, 1H), 7.62-7.57 (m, 1H), 5.71-5.60 (m, 4H), 5.01-4.91 (m, 1H), 4.65-4.55 (m, 1H), 4.41-4.27 (m, 2H), 4.24-4.14 (m, 5H), 4.12-3.92 (m, 2H), 3.66-3.48 (m, 7H), 3.39 (t, J = 6.1 Hz, 4H), 3.29-3.24 (m, 8H), 2.37-2.27 (m, 1H), 2.25-2.16 (m, 1H), 2.11-2.02 (m, 4H), 1.89-1.84 (m, 4H), 1.69-1.58 (m, 1H), 1.47-1.33 (m, 1H), 0.87-0.73 (m, 2H), 0.04-−0.02 (m, 1H). |
| 170 | di-tert-butyl ((((difluoro(2- (((3S,6S,7aS, 8aR,9aR)-3-(3- (morpholine-4- carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) phosphoryl) bis(oxy)) bis(methylene)) bis(carbonate) | 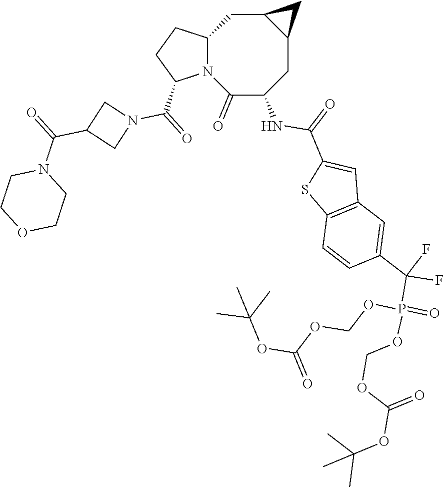 [SMILES: CC(C)(C)OC(=O)OCOP(=O)(OCOC(=O)OC(C)(C)C)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 941.2 [M + H]⁺ | ¹H NMR (400 MHz, CD₃CN) δ 8.15-8.07 (m, 1H), 8.04-7.96 (m, 2H), 7.96-7.87 (m, 1H), 7.63-7.55 (m, 1H), 5.68-5.56 (m, 4H), 4.97 (q, J = 7.5 Hz, 1H), 4.65-4.56 (m, 1H), 4.43-4.37 (m, 1H), 4.37-4.29 (m, 1H), 4.25-4.16 (m, 1H), 4.12-3.90 (m, 2H), 3.65-3.39 (m, 7H), 3.30-3.14 (m, 2H), 2.37-2.27 (m, 1H), 2.26-2.17 (m, 1H), 2.12-2.02 (m, 3H), 1.92-1.86 (m, 1H), 1.69-1.58 (m, 1H), 1.50-1.36 (m, 20H), 0.87-0.73 (m, 2H), 0.04-−0.02 (m, 1H). |
| 171 | S,S′- ((((difluoro(2- (((3S,6S,7aS, 8aR,9aR)-3-(3- (morpholine-4- carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) phosphoryl) bis(oxy)) bis(ethane-2,1- diyl)) bis(2,2- dimethyl- propanethioate) | 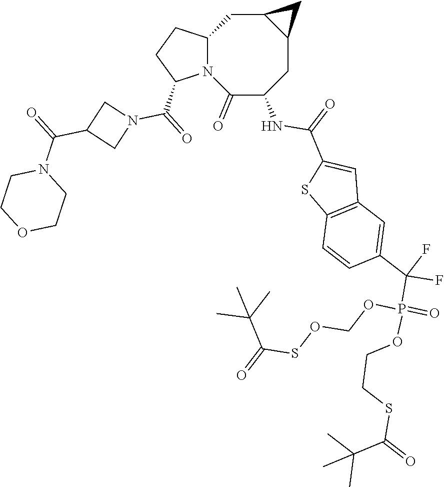 [SMILES: CC(C)(C)C(=O)SCCOP(=O)(OCOSC(=O)C(C)(C)C)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 969.1 [M + H]⁺ | ¹H NMR (400 MHz, CD₃OD) δ 8.19-8.10 (m, 2H), 8.08-7.97 (m, 1H), 7.67-7.60 (m, 1H), 5.08-5.01 (m, 1H), 4.76-4.69 (m, 1H), 4.55-4.38 (m, 2H), 4.33-4.14 (m, 6H), 4.10-4.03 (m, 1H), 3.81-3.71 (m, 1H), 3.67-3.41 (m, 6H), 3.37-3.33 (m, 1H), 3.30-3.20 (m, 1H), 3.18-3.09 (m, 4H), 2.50-2.38 (m, 1H), 2.35-2.05 (m, 4H), 2.04-1.91 (m, 2H), 1.80-1.66 (m, 1H), 1.46-1.34 (m, 1H), 1.20 (s, 18H), 0.98-0.77 (m, 2H), 0.09-0.05 (m, 1H). |
| 172 | (((difluoro(2- (((3S,6S,7aS, 8aR,9aR)-3-(3- (morpholine-4- carbonyl) azetidine-1- carbonyl)-5- oxodecahydro- 1H-cyclopropa [d]pyrrolo[1,2- a]azocin-6- yl)carbamoyl) benzo[b] thiophen-5- yl)methyl) phosphoryl) bis(oxy)) bis(methylene) bis(2- methoxyethyl) bis(carbonate) | 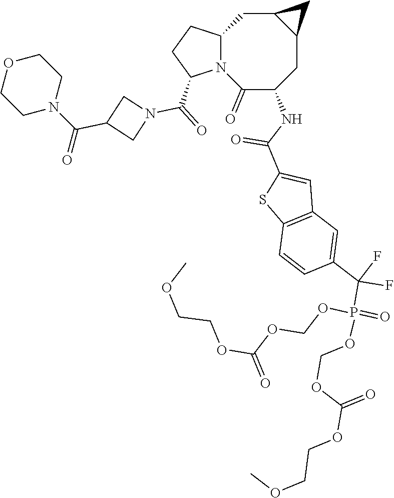 [SMILES: COCCOC(=O)OCOP(=O)(OCOC(=O)OCCOC)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 945.3 [M + H]⁺ | ¹H NMR (400 MHz, CD₃CN) δ 8.16-8.08 (m, 1H), 8.08-7.99 (m, 2H), 7.82-7.70 (m, 1H), 7.65-7.55 (m, 1H), 5.73-5.58 (m, 4H), 5.01-4.89 (m, 1H), 4.64-4.52 (m, 1H), 4.41-4.14 (m, 7H), 4.11-3.80 (m, 3H), 3.65-3.46 (m, 11H), 3.35-3.27 (m, 6H), 3.27-3.19 (m, 2H), 2.37-2.26 (m, 1H), 2.24-2.17 (m, 1H), 2.11-2.04 (m, 3H), 1.69-1.57 (m, 1H), 1.47-1.38 (m, 1H), 0.91-0.72 (m, 2H), 0.07-−0.09 (m, 1H). |
| 173 | benzyl ((difluoro(2- (((3S,6S,7aS, 8aR,9aR)-3-(3- (morpholine-4- carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) (phenoxy) phosphoryl)-L- alaninate | 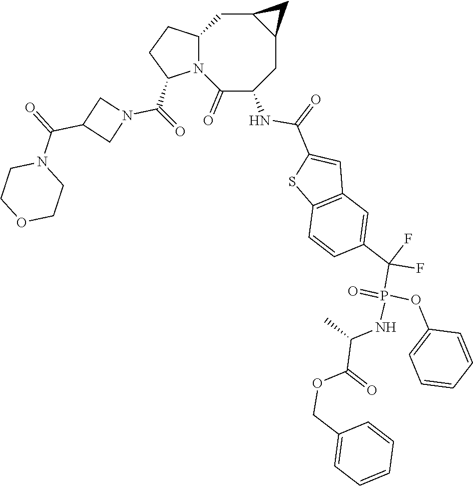 [SMILES: C[C@H](NP(=O)(Oc1ccccc1)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1)C(=O)OCc1ccccc1] | 918.1 [M + H]⁺ | ¹H NMR (400 MHz, CD₃OD) δ 8.21-8.11 (m, 2H), 8.08-7.93 (m, 1H), 7.74-7.65 (m, 1H), 7.35-7.24 (m, 7H), 7.21-7.10 (m, 3H), 5.12-4.95 (m, 3H), 4.75-4.67 (m, 1H), 4.52-4.36 (m, 2H), 4.32-4.16 (m, 2H), 4.10-3.90 (m, 2H), 3.82-3.44 (m, 8H), 3.39-3.34 (m, 1H), 2.49-2.37 (m, 1H), 2.34-2.06 (m, 4H), 2.02-1.89 (m, 2H), 1.79-1.66 (m, 1H), 1.45-1.31 (m, 1H), 1.29-1.26 (m, 3H), 0.99-0.86 (m, 1H), 0.87-0.79 (m, 1H), 0.08-0.03 (m, 1H). |
| 174 | benzyl (2S)- 2- (((difluoro(2- (((3S,6S,7aS, 8aR,9aR)-3-(3- (morpholine-4- carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) (phenoxy) phosphoryl)oxy) propanoate | 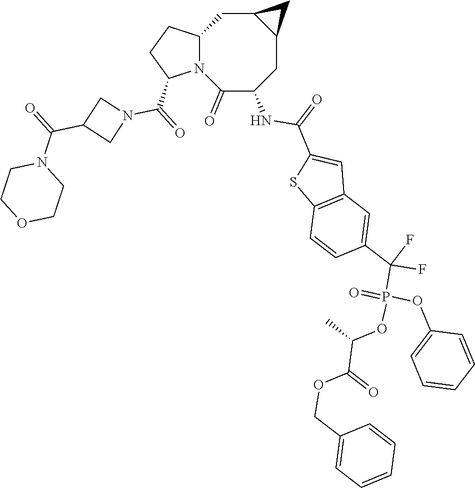 [SMILES: C[C@H](OP(=O)(Oc1ccccc1)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1)C(=O)OCc1ccccc1] | 919.1 [M + H]⁺ | ¹H NMR (400 MHz, CD₃OD) δ 8.24-7.88 (m, 3H), 7.69-7.62 (m, 1H), 7.48-6.91 (m, 10H), 5.20-4.96 (m, 4H), 4.75-4.68 (m, 1H), 4.55-4.36 (m, 2H), 4.33-4.16 (m, 2H), 4.08-4.04 (m, 1H), 3.82-3.41 (m, 7H), 3.37-3.32 (m, 1H), 3.29-3.16 (m, 1H), 2.51-2.38 (m, 1H), 2.35-2.07 (m, 4H), 2.04-1.89 (m, 2H), 1.79-1.66 (m, 1H), 1.53-1.35 (m, 4H), 1.00-0.78 (m, 2H), 0.08-0.04 (m, 1H). |
| 175 | ((((2- (((3S,6S,7aS, 8aR,9aR)-3- ([1,1′- biphenyl]-4- ylcarbamoyl)-5- oxodecahydro- 1H- cyclopropa[d] pyrrolo[1,2- a]azocin-6- yl)carbamoyl) benzo[b] thiophen-5- yl)difluoro- methyl) phosphoryl) bis(oxy)) bis(methylene) bis(2,2- dimethyl- propanoate) | 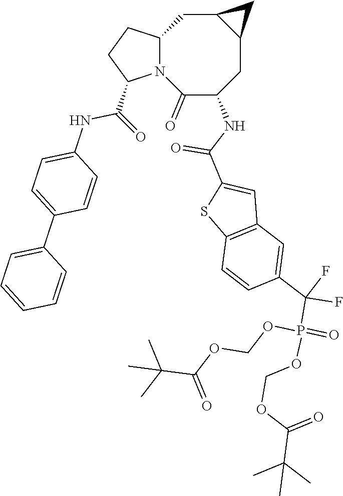 [SMILES: CC(C)(C)C(=O)OCOP(=O)(OCOC(=O)C(C)(C)C)C(F)(F)c1ccc2sc(C(=O)N[C@H]3C[C@@H]4C[C@@H]4C[C@H]4CC[C@@H](C(=O)Nc5ccc(-c6ccccc6)cc5)N4C3=O)cc2c1] | 908.4 [M + H]⁺ | ¹H NMR (400 MHz, CD₃OD) δ 8.19 (s, 1H), 8.13 (s, 1H), 8.08 (d, J = 8.6 Hz, 1H), 7.63- 7.53 (m, 7H), 7.45- 7.38 (m, 2H), 7.33- 7.26 (m, 1H), 5.80- 5.67 (m, 4H), 5.10 (t, J = 8.9 Hz, 1H), 4.83- 4.75 (m, 1H), 4.39- 4.23 (m, 1H), 2.60- 2.44 (m, 1H), 2.35- 2.24 (m, 3H), 2.21- 2.09 (m, 2H), 2.04- 1.95 (m 1H), 1.80- 1.68 (m, 1H), 1.23- 1.20 (m, 2H), 1.18 (s, 18H), 0.96-0.89 (m, 1H), 0.86-0.78 (m, 1H). |
| 176 | (((difluoro(2- (((3S,6S,7aS, 8aR,9aR)-3-(3- (morpholine-4- carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) phosphoryl) bis(oxy)) bis(methylene) diisopropyl bis(carbonate) | 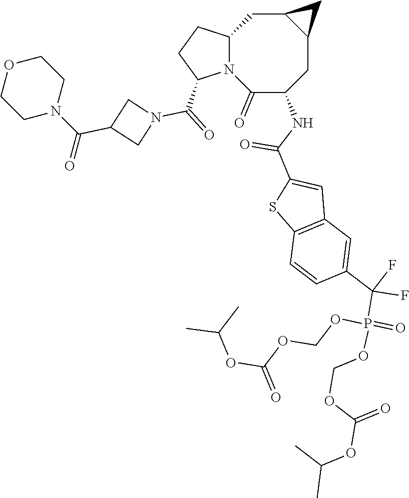 [SMILES: CC(C)OC(=O)OCOP(=O)(OCOC(=O)OC(C)C)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 913.3 [M + H]⁺ | ¹H NMR (400 MHz, CD₃CN) δ 8.09 (s, 1H), 8.03-7.90 (m, 3H), 7.58 (d, J = 8.6 Hz, 1H), 5.70-5.60 (m, 4H), 4.98 (q, J = 7.3 Hz, 1H), 4.92-4.80 (m, 2H), 4.66-4.55 (m, 1H), 4.44-4.30 (m, 2H), 4.23-4.19 (m, 1H), 4.11-3.91 (m, 2H), 3.63-3.42 (m, 7H), 3.25-3.15 (m, 2H), 2.38-2.28 (m, 1H), 2.25-2.15 (m, 1H), 2.12-2.01 (m, 3H), 1.92-1.84 (m, 1H), 1.68-1.59 (m, 1H), 1.48-1.37 (m, 1H), 1.30-1.22 (m, 13H), 0.86-0.73 (m, 2H), 0.04-−0.04 (m, 1H). |
| 177 | diethyl 4,4′- (((difluoro(2- (((3S,6S,7aS, 8aR,9aR)-3-(3- (morpholine-4- carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) phosphoryl) bis(oxy)) dibutyrate | 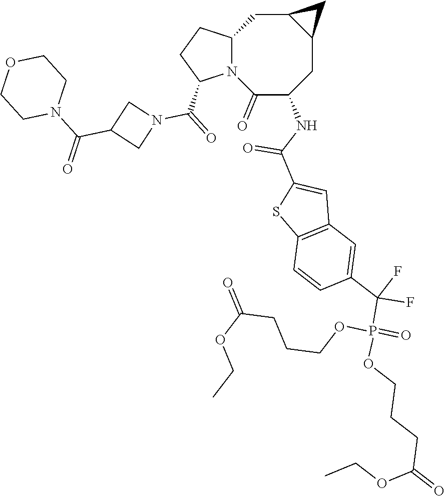 [SMILES: CCOC(=O)CCCOP(=O)(OCCCC(=O)OCC)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 909.4 [M + H]⁺ | ¹H NMR (400 MHz, CD₃CN) δ 8.16-8.10 (m, 1H), 8.07-8.02 (m, 1H), 8.01-7.98 (m, 1H), 7.81-7.72 (m, 1H), 7.67-7.58 (m, 1H), 5.03-4.89 (m, 1H), 4.65-4.55 (m, 1H), 4.41-4.27 (m, 2H), 4.24-3.85 (m, 12H), 3.67-3.43 (m, 7H), 3.31-3.21 (m, 2H), 2.39-2.26 (m, 5H), 2.24-2.15 (m, 1H), 2.11-1.97 (m, 4H), 1.91-1.86 (m, 4H), 1.70-1.55 (m, 1H), 1.49-1.36 (m, 1H), 1.19 (t, J = 7.1 Hz, 6H), 0.88-0.71 (m, 2H), 0.07-−0.05 (m, 1H). |
| 178 | S,S′- ((((difluoro(2- (((3S,6S,7aS, 8aR,9aR)-3-(3- (morpholine-4- carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) phosphoryl) bis(oxy)) bis(ethane-2,1- diyl)) dibutane- thioate | 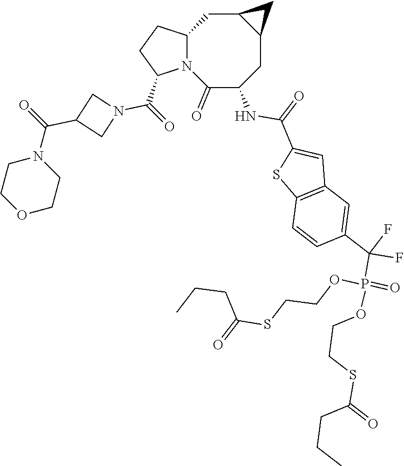 [SMILES: CCCC(=O)SCCOP(=O)(OCCSC(=O)CCC)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 941.3 [M + H]⁺ | ¹H NMR (400 MHz, CD₃OD) δ 8.21-8.11 (m, 2H), 8.10-7.98 (m, 1H), 7.67-7.60 (m, 1H), 5.07-5.02 (m, 1H), 4.76-4.68 (m, 1H), 4.54-4.38 (m, 2H), 4.33-4.17 (m, 6H), 4.10-4.05 (m, 1H), 3.82-3.72 (m, 1H), 3.67-3.47 (m, 6H), 3.38-3.34 (m, 1H), 3.28-3.05 (m, 5H), 2.54 (t, J = 7.3 Hz, 3H), 2.49-2.09 (m, 6H), 2.03-1.91 (m, 2H), 1.78-1.70 (m, 1H), 1.69-1.58 (m, 4H), 1.45-1.35 (m, 1H), 0.97-0.89 (m, 7H), 0.87-0.82 (m, 1H), 0.09-0.5 (m, 1H). |
| 179 | S,S′-(((((2- (((3S,6S,7aR, 8aS,9aR)-3-(3- (1H-imidazol-1- yl)azetidine-1- carbonyl)-5- oxodecahydro- 1H- cyclopropa[d] pyrrolo[1,2- a]azocin-6- yl)carbamoyl) benzo[b] thiophen-5- yl) difluoromethyl) phosphoryl) bis(oxy)) bis(ethane-2,1- diyl)) bis(3- methylbutane thioate) | 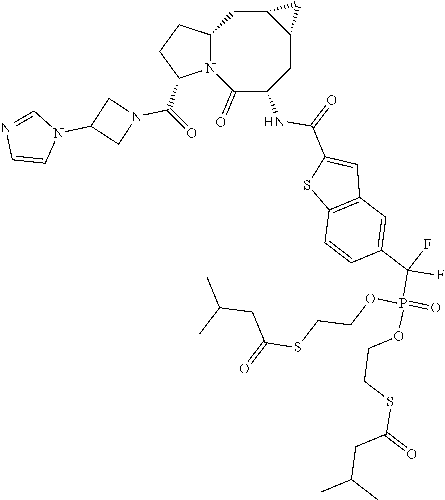 [SMILES: CC(C)CC(=O)SCCOP(=O)(OCCSC(=O)CC(C)C)C(F)(F)c1ccc2sc(C(=O)N[C@H]3C[C@H]4C[C@H]4C[C@H]4CC[C@@H](C(=O)N5CC(n6ccnc6)C5)N4C3=O)cc2c1] | 922.2 [M + H]⁺ | ¹H NMR (400 MHz, CD₃OD) δ 8.21-8.17 (m, 2H), 8.10-8.08 (m, 1H), 7.90-7.83 (m, 1H), 7.65 (d, J = 8.8 Hz, 1H), 7.56-7.43 (m, 1H), 7.06-7.02 (m, 1H), 5.28-5.17 (m, 1H), 5.13-5.02 (m, 1H), 4.84-4.75 (m, 1H), 4.61-4.08 (m, 9H), 3.23-3.11 (m, 4H), 2.53-2.39 (m, 5H), 2.36-1.94 (m, 8H), 1.81-1.68 (m, 1H), 1.49-1.35 (m, 1H), 1.02-0.95 (m, 1H), 0.94-0.91 (m, 12H), 0.89-0.82 (m, 1H), 0.10-0.06 (m, 1H). |
| 180 | S,S′-(((((2- (((3S,6S,7aR, 8aS,9aR)-3-(3- (1H-imidazol-1- yl)azetidine-1- carbonyl)-5- oxodecahydro- 1H- cyclopropa[d] pyrrolo[1,2- a]azocin-6- yl)carbamoyl) benzo[b] thiophen-5- yl) difluoromethyl) phosphoryl) bis(oxy)) bis(ethane-2,1- diyl)) bis(2,2- dimethyl- propanethioate) | 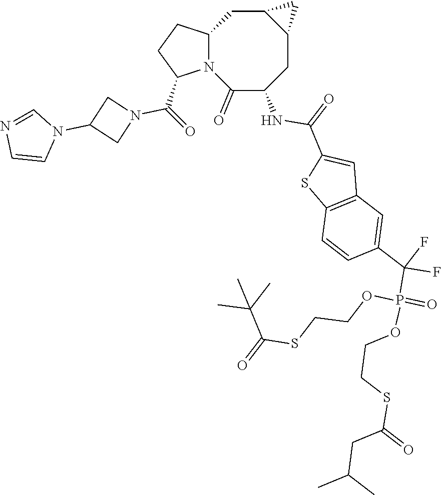 [SMILES: CC(C)CC(=O)SCCOP(=O)(OCCSC(=O)C(C)(C)C)C(F)(F)c1ccc2sc(C(=O)N[C@H]3C[C@H]4C[C@H]4C[C@H]4CC[C@@H](C(=O)N5CC(n6ccnc6)C5)N4C3=O)cc2c1] | 922.3 [M + H]⁺ | ¹H NMR (400 MHz, CD₃OD) δ 8.36-8.23 (m, 1H), 8.23-8.16 (m, 2H), 8.11 (d, J = 8.6 Hz, 1H), 7.80-7.71 (m, 0.5H), 7.67 (d, J = 8.6 Hz, 1H), 7.64-7.56 (m, 0.5H), 7.31-7.16 (m, 1H), 5.35-5.25 (m, 1H), 5.04 (t, J = 8.9 Hz, 0.5H), 5.00-4.92 (m, 1H), 4.82-4.74 (m, 1H), 4.64-4.55 (m, 0.5H), 4.53-4.39 (m, 2H), 4.34 (br. s., 1H), 4.30-4.13 (m, 5H), 3.18-3.08 (m, 4H), 2.38 (dd, J = 15.2, 4.6 Hz, 1H), 2.33-2.12 (m, 3H), 2.05-1.83 (m, 3H), 1.77-1.65 (m, 1H), 1.54-1.39 (m, 1H), 1.35-1.26 (m, 1H), 1.20 (s, 18H), 0.92-0.82 (m, 1H), 0.19 (q, J = 5.0 Hz, 1H). |
| 181 | S,S′-(((((2- (((3S,6S,7aS, 8aR,9aR)-3-(3- (1H-imidazol-1- yl)azetidine- 1-carbonyl)-5- oxodecahydro- 1H- cyclopropa[d] pyrrolo[1,2- a]azocin-6- yl)carbamoyl) benzo[b] thiophen-5- yl) difluoromethyl) phosphoryl) bis(oxy)) bis(ethane-2,1- diyl)) bis(2,2- dimethyl- propanethioate) | 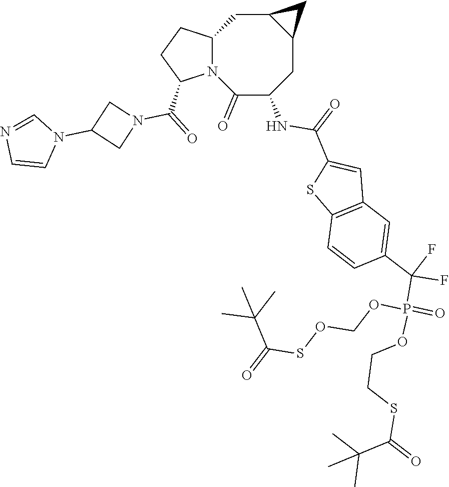 [SMILES: CC(C)(C)C(=O)SCCOP(=O)(OCOSC(=O)C(C)(C)C)C(F)(F)c1ccc2sc(C(=O)N[C@H]3C[C@@H]4C[C@@H]4C[C@H]4CC[C@@H](C(=O)N5CC(n6ccnc6)C5)N4C3=O)cc2c1] | 922.2 [M + H]⁺ | ¹H NMR (400 MHz, CD₃OD) δ 8.21-8.18 (m, 2H), 8.09 (d, J = 8.3 Hz, 1H), 7.88-7.86 (m, 1H), 7.68-7.44 (m, 2H), 7.06-7.02 (m, 1H), 5.28-5.17 (m, 1H), 5.13-5.02 (m, 1H), 4.83-4.74 (m, 1H), 4.59-4.10 (m, 9H), 3.19-3.09 (m, 4H), 2.54-2.41 (m, 1H), 2.36-2.09 (m, 4H), 2.08-1.93 (m, 2H), 1.81-1.68 (m, 1H), 1.47-1.34 (m, 1H), 1.19 (d, J = 3.7 Hz, 18H), 1.05-0.92 (m, 1H), 0.88-0.83 (m, 1H), 0.10-0.06 (m, 1H). |
| 182 | ((((S) or (R)- fluoro(2- (((3S,6S,7aS, 8aR,9aR)-3-(3- (morpholine-4- carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) phosphoryl) bis(oxy)) bis(methylene) bis(2,2- dimethyl- propanoate) | 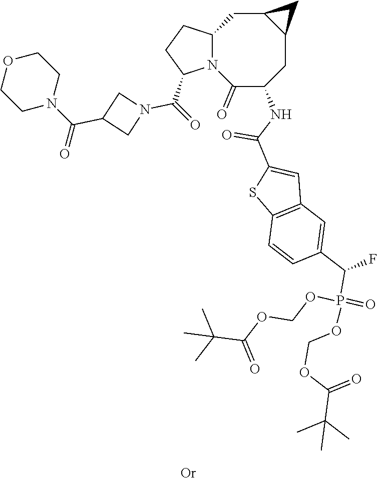 [SMILES: CC(C)(C)C(=O)OCOP(=O)(OCOC(=O)C(C)(C)C)[C@@H](F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 891.2 [M + H]⁺ | ¹H NMR (400 MHz, CD₃CN) δ 8.00-7.92 (m, 3H), 7.83-7.72 (m, 1H), 7.56-7.46 (m, 1H), 5.99 (dd, J = 43.8, 7.1 Hz, 1H), 5.67-5.53 (m, 4H), 4.96 (q, J = 7.8 Hz, 1H), 4.60 (q, J = 8.6 Hz, 1H), 4.42- 4.27 (m, 2H), 4.25- 4.15 (m, 1H), 4.12- 3.90 (m, 2H), 3.68- 3.43 (m, 7H), 3.30- 3.14 (m, 2H), 2.37- 2.26 (m, 1H), 2.24- 2.17 (m, 1H), 2.10- 2.02 (m, 3H), 1.92- 1.84 (m, 2H), 1.70- 1.57 (m, 1H), 1.49- 1.36 (m, 1H), 1.20- 1.13 (m, 18H), 0.86- 0.72 (m, 2H), 0.06- −0.04 (m, 1H). |
| | | 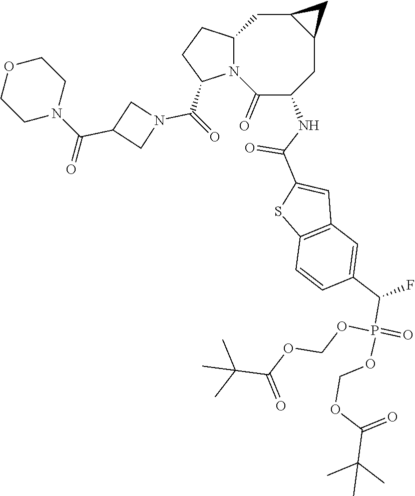 [SMILES: CC(C)(C)C(=O)OCOP(=O)(OCOC(=O)C(C)(C)C)[C@@H](F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | | |
| | | SFC Peak 2 was used for | | |
| | | biological testing and/or further | | |
| | | synthesis | | |
| 183 | ((((R) or (S)- fluoro(2- (((3S,6S,7aS, 8aR,9aR)-3-(3- (morpholine-4- carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) phosphoryl) bis(oxy)) bis(methylene) bis(2,2- dimethyl- propanoate) | 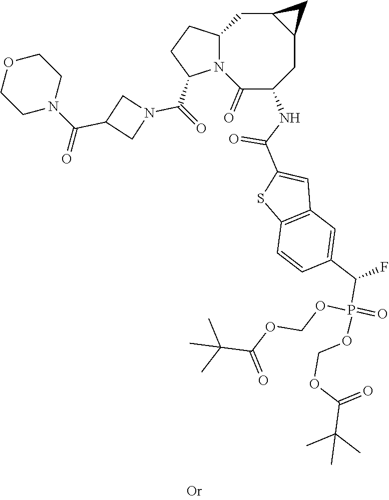 [SMILES: CC(C)(C)C(=O)OCOP(=O)(OCOC(=O)C(C)(C)C)[C@@H](F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 891.2 [M + H]⁺ | ¹H NMR (400 MHz, CD₃CN) δ 8.03-7.90 (m, 3H), 7.80-7.64 (m, 1H), 7.59-7.43 (m, 1H), 5.99 (dd, J = 43.8, 7.3 Hz, 1H), 5.69-5.52 (m, 4H), 5.00-4.91 (m, 1H), 4.65-4.54 (m, 1H), 4.42-4.26 (m, 2H), 4.24-4.14 (m, 1H), 4.12-3.91 (m, 2H), 3.67-3.43 (m, 7H), 3.31-3.18 (m, 2H), 2.38-2.26 (m, 1H), 2.24-2.17 (m, 1H), 2.10-2.01 (m, 3H), 1.91-1.81 (m, 2H), 1.70-1.57 (m, 1H), 1.49-1.36 (m, 1H), 1.19-1.10 (m, 18H), 0.87-0.72 (m, 2H), 0.06-−0.05 (m, 1H). |
| | | 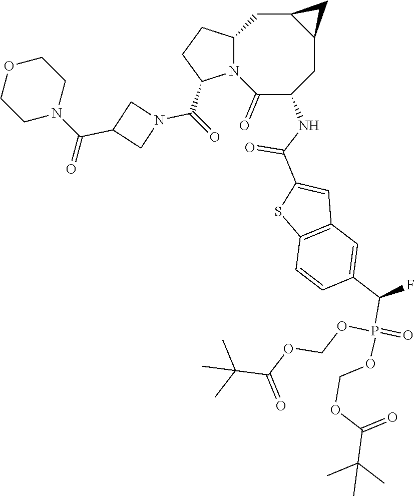 [SMILES: CC(C)(C)C(=O)OCOP(=O)(OCOC(=O)C(C)(C)C)[C@H](F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | | |
| | | SFC Peak 1 was used for | | |
| | | biological testing and/or further | | |
| | | synthesis | | |
| 184 | isopropyl (2S)-3- (((difluoro(2- (((3S,6S,7aS, 8aR,9aR)-3-(3- (morpholine-4- carbonyl) azetidine-1- carbonyl)-5- oxodecahydro- 1H-cyclopropa [d]pyrrolo[1,2- a]azocin-6- yl)carbamoyl) benzo[b] thiophen-5- yl)methyl) (phenoxy) phosphoryl) amino)-2- methyl- propanoate | 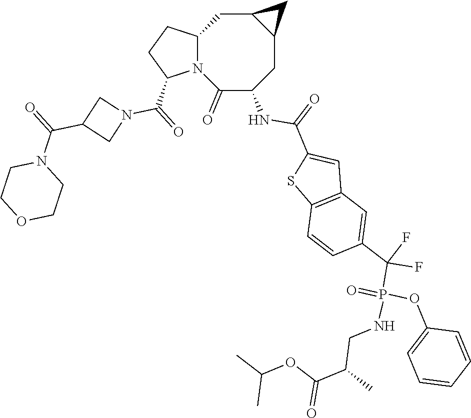 [SMILES: CC(C)OC(=O)[C@@H](C)CNP(=O)(Oc1ccccc1)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 884.2 [M + H]⁺ | ¹H NMR (400 MHz, CD₃OD) δ 8.20-8.12 (m, 2H), 8.08-7.99 (m, 1H), 7.71-7.68 (m, 1H), 7.36-7.29 (m, 2H), 7.21-7.09 (m, 3H), 5.05-4.49 (m, 1H), 4.94-4.87 (m, 1H), 4.73-4.51 (m, 1H), 4.53-4.37 (m, 2H), 4.32-4.15 (m, 2H), 4.11-4.02 (m, 1H), 3.82-3.71 (m, 1H), 3.68-3.44 (m, 6H), 3.39-3.34 (m, 1H), 3.30-3.11 (m, 2H), 3.01-2.88 (m, 1H), 2.50-2.37 (m, 2H), 2.34-2.07 (m, 4H), 2.03-1.90 (m, 2H), 1.78-1.67 (m, 1H), 1.46-1.36 (m, 1H), 1.19-1.12 (m, 6H), 1.05-0.97 (m, 3H), 0.96-0.88 (m, 1H), 0.85-0.83 (m, |
| | | | | 1H), 0.08-0.05 (m, 1H). |
| 185 | S-(2- (((difluoro(2- (((3S,6S,7aS, 8aR,9aR)-3-(3- (morpholine-4- carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) (phenoxy) phosphoryl) oxy)ethyl) 3- methylbutane thioate | 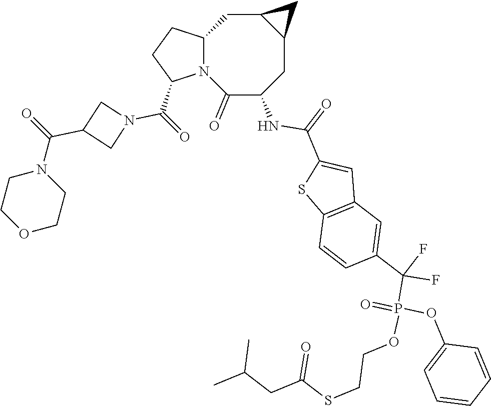 [SMILES: CC(C)CC(=O)SCCOP(=O)(Oc1ccccc1)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 901.1 [M + H]⁺ | ¹H NMR (400 MHz, CD₃OD) δ 8.23-8.15 (m, 2H), 8.12-7.99 (m, 1H), 7.70-7.68 (m, 1H), 7.39-7.34 (m, 2H), 7.27-7.21 (m, 1H), 7.16-7.14 (m, 2H), 5.07-5.00 (m, 1H), 4.73-4.69 (m, 1H), 4.53-4.37 (m, 2H), 4.34-4.16 (m, 4H), 4.11-4.02 (m, 1H), 3.83-3.71 (m, 1H), 3.69-3.42 (m, 6H), 3.39-3.33 (m, 2H), 3.16-3.10 (m, 2H), 2.49-1.89 (m, 10H), 1.79-1.67 (m, 1H), 1.47-1.27 (m, 1H), 0.98-0.92 (m, 1H), 0.90 (d, J = 6.6 Hz, 6H), 0.87-0.83 (m, 1H), 0.10-0.03 (m, 1H). |
| 186 | isopropyl ((difluoro(2- (((3S,6S,7aS, 8aR,9aR)-3- (3-(morpholine- 4- carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) (phenoxy) phosphoryl)-L- alaninate | 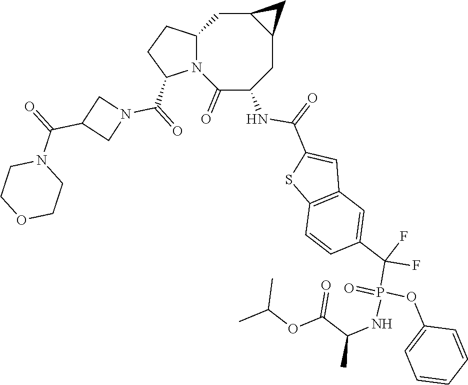 [SMILES: CC(C)OC(=O)[C@H](C)NP(=O)(Oc1ccccc1)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 870.1 [M + H]⁺ | ¹H NMR (400 MHz, CD₃OD) δ 8.23-8.15 (m, 2H), 8.09-8.00 (m, 1H), 7.74-7.72 (m, 1H), 7.37-7.28 (m, 2H), 7.22-7.11 (m, 3H), 5.05-5.01 (m, 1H), 4.97-4.87 (m, 2H), 4.76-4.66 (m, 1H), 4.52-4.37 (m, 2H), 4.33-4.16 (m, 2H), 4.11-4.02 (m, 1H), 3.93-3.71 (m, 2H), 3.68-3.49 (m, 6H), 3.39-3.35 (m, 1H), 2.49-2.39 (m, 1H), 2.34-2.07 (m, 4H), 2.04-1.91 (m, 2H), 1.79-1.67 (m, 1H), 1.46-1.35 (m, 1H), 1.27 (t, J = 8.1 Hz, 3H), 1.21-1.10 (m, 6H), 1.00-0.89 (m, 1H), 0.87-0.81 (m, |
| | | | | 1H), 0.08-0.03 (m, 1H). |
| 187 | (((difluoro(2- (((3S,6S,7aR, 8aS,9aR)-5- oxo-3-(3- (pyridin-3- yl)azetidine-1- carbonyl) decahydro-1H- cyclopropa[d] pyrrolo[1,2- a]azocin-6- yl)carbamoyl) benzo[b] thiophen-5- yl)methyl) phosphoryl) bis(oxy)) bis(methylene) bis(2,2- dimethyl- propanoate) | 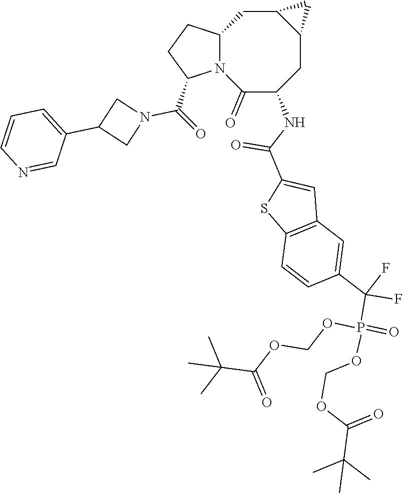 [SMILES: CC(C)(C)C(=O)OCOP(=O)(OCOC(=O)C(C)(C)C)C(F)(F)c1ccc2sc(C(=O)N[C@H]3C[C@H]4C[C@H]4C[C@H]4CC[C@@H](C(=O)N5CC(c6cccnc6)C5)N4C3=O)cc2c1] | 873.2 [M + H]⁺ | ¹H NMR (400 MHz, CD₃CN) δ 8.59-8.54 (m, 1H), 8.50-8.43 (m, 1H), 8.17-8.10 (m, 1H), 8.08-8.03 (m, 1H), 8.01-7.68 (m, 3H), 7.62-7.59 (m, 1H), 7.37-7.33 (m, 1H), 5.72-5.61 (m, 4H), 4.93-4.58 (m, 2H), 4.53-4.34 (m, 2H), 4.33-4.15 (m, 2H), 3.99-3.84 (m, 2H), 2.30-2.18 (m, 2H), 2.11-2.02 (m, 2H), 1.91-1.74 (m, 2H), 1.70-1.56 (m, 1H), 1.59-1.40 (m, 1H), 1.33-1.19 (m, 2H), 1.18-1.14 (m, 18H), 0.82-0.76 (m, 1H), 0.14-0.10 (m, 1H). |
| 188 | S,S′-(((((2- (((3S,6S,7aS, 8aR,9aR)-3-(3- (1H-imidazol-1- yl)azetidine- 1-carbonyl)-5- oxodecahydro- 1H- cyclopropa[d] pyrrolo[1,2- a]azocin-6- yl)carbamoyl) benzo[b] thiophen-5- yl) difluoromethyl) phosphoryl) bis(oxy)) bis(ethane- 2,1-diyl)) dibutanethioate | 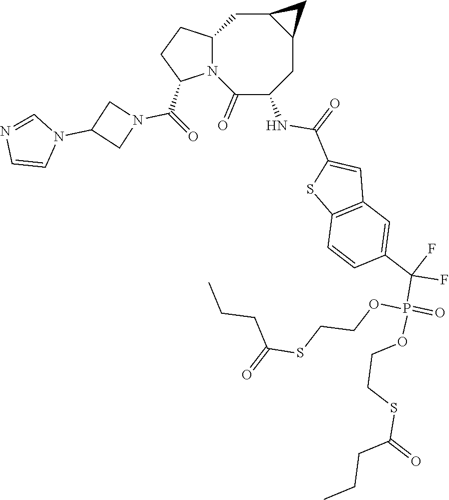 [SMILES: CCCC(=O)SCCOP(=O)(OCCSC(=O)CCC)C(F)(F)c1ccc2sc(C(=O)N[C@H]3C[C@@H]4C[C@@H]4C[C@H]4CC[C@@H](C(=O)N5CC(n6ccnc6)C5)N4C3=O)cc2c1] | 894.1 [M + H]⁺ | ¹H NMR (400 MHz, CD₃OD) δ 8.21-8.17 (m, 2H), 8.09 (d, J = 8.8 Hz, 1H), 7.89-7.86 (m, 1H), 7.67-7.43 (m, 2H), 7.07-7.03 (m, 1H), 5.27-5.18 (m, 1H), 5.12-5.02 (m, 1H), 4.84-4.75 (m, 1H), 4.60-4.10 (m, 9H), 3.23-3.09 (m, 4H), 2.57-2.42 (m, 5H), 2.36-2.11 (m, 4H), 2.09-1.94 (m, 2H), 1.81-1.69 (m, 1H), 1.69-1.58 (m, 4H), 1.48-1.38 (m, 1H), 1.02-0.81 (m, 8H), 0.10-0.06 (m, 1H). |
| 189 | (((difluoro(2- (((3S,6S,7aR, 8aS,9aR)-5- oxo-3-(3- (pyridin-3- yl)azetidine-1- carbonyl) decahydro-1H- cyclopropa[d] pyrrolo[1,2- a]azocin-6- yl)carbamoyl) benzo[b] thiophen-5- yl)methyl) phosphoryl) bis(oxy)) bis(methylene) diisopropyl bis(carbonate) | 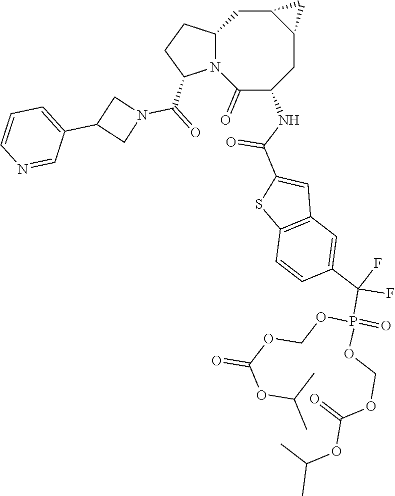 [SMILES: CC(C)OC(=O)OCOP(=O)(OCOC(=O)OC(C)C)C(F)(F)c1ccc2sc(C(=O)N[C@H]3C[C@H]4C[C@H]4C[C@H]4CC[C@@H](C(=O)N5CC(c6cccnc6)C5)N4C3=O)cc2c1] | 877.1 [M + H]⁺ | ¹H NMR (400 MHz, CD₃CN) δ 8.61-8.52 (m, 1H), 8.50-8.43 (m, 1H), 8.18-8.10 (m, 1H), 8.10-8.04 (m, 1H), 8.03-7.97 (m, 1H), 7.96-7.90 (m, 1H), 7.89-7.75 (m, 1H), 7.60 (d, J = 8.6 Hz, 1H), 7.43-7.29 (m, 1H), 5.73-5.56 (m, 4H), 4.92-4.82 (m, 3H), 4.81-4.58 (m, 1H), 4.50-4.33 (m, 2H), 4.32-4.15 (m, 2H), 4.01-3.84 (m, 2H), 2.28-2.17 (m, 2H), 2.11-1.99 (m, 3H), 1.83-1.74 (m, 1H), 1.70-1.56 (m, 1H), 1.56-1.44 (m, 1H), 1.34-1.19 (m, 14H), 0.85-0.71 (m, 1H), 0.17-0.04 (m, 1H). |
| 190 | isopropyl ((4- chlorophenoxy) ((2-(((3S,6S,7aS, 8aR,9aR)-3-(3- (morpholine-4- carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) phosphoryl)-L- alaninate | 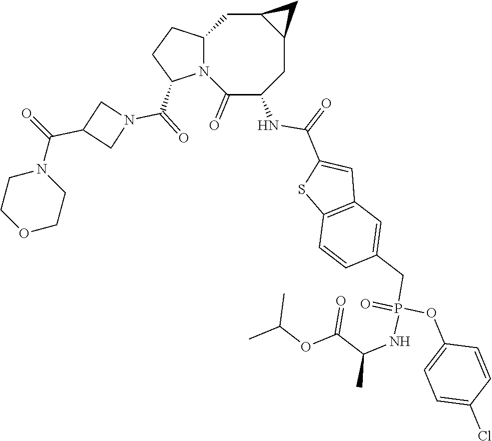 [SMILES: CC(C)OC(=O)[C@H](C)NP(=O)(Cc1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1)Oc1ccc(Cl)cc1] | 868.1 [M + H]⁺ | ¹H NMR (400 MHz, DMSO-d₆) δ 8.88- 8.77 (m, 1H), 8.23 (s, 1H), 7.94 (t, J = 6.7 Hz, 1H), 7.88 (br. s., 1H), 7.45-7.36 (m, 3H), 7.15 (dd, J =14.1, 8.4 Hz, 2H), 5.76-5.59 (m, 1H), 4.98-4.87 (m, 1H), 4.85-4.72 (m, 1H), 4.62 (t, J = 8.6 Hz, 0.5H), 4.51 (t, J = 7.2 Hz, 0.5H), 4.39-4.25 (m, 2H), 4.21-4.11 (m, 1H), 4.05-3.85 (m, 2H), 3.84-3.61 (m, 2H), 3.59-3.38 (m, 8H), 3.25 (br. s., 2H), 2.29 (br. s., 1H), 2.20- 1.92 (m, 4H), 1.90- 1.74 (m, 2H), 1.70- 1.55 (m, 1H), 1.40- 0.97 (m, 10H), 0.84- 0.76 (m, 1H), 0.74- 0.68 (m, 1H), 0.03- −0.07 (m, 1H). |
| 191 | isopropyl ((2- chlorophenoxy) ((2-(((3S,6S, 7aS,8aR,9aR)- 3-(3- (morpholine-4- carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) phosphoryl)-L- alaninate | 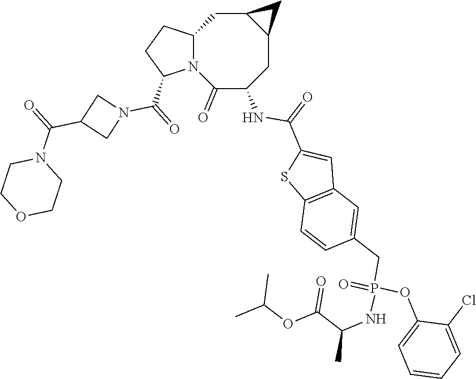 [SMILES: CC(C)OC(=O)[C@H](C)NP(=O)(Cc1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1)Oc1ccccc1Cl] | 868.1 [M + H]⁺ | ¹H NMR (400 MHz, CD₃OD) δ 8.07-8.02 (m, 1H), 7.95-7.90 (m, 1H), 7.88-7.81 (m, 1H), 7.54-7.35 (m, 3H), 7.27-7.19 (m, 1H), 7.16-7.10 (m, 1H), 5.05-5.00 (m, 1H), 4.93-4.86 (m, 2H), 4.75-4.68 (m, 1H), 4.52-4.37 (m, 2H), 4.32-4.17 (m, 2H), 4.10-4.03 (m, 1H), 3.93-3.72 (m, 2H), 3.66-3.52 (m, 8H), 3.38-3.34 (m, 1H), 2.48-2.39 (m, 1H), 2.34-2.07 (m, 4H), 2.02-1.90 (m, 2H), 1.79-1.67 (m, 1H), 1.45-1.35 (m, 1H), 1.21-1.07 (m, 9H), 0.98-0.89 (m, 1H), 0.87-0.79 (m, |
| | | | | 1H), 0.08-0.02 (m, 1H). |
| 192 | ethyl ((difluoro(2- (((3S,6S,7aS, 8aR,9aR)-3-(3- (morpholine-4- carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) (phenoxy) phosphoryl)-L- alaninate | 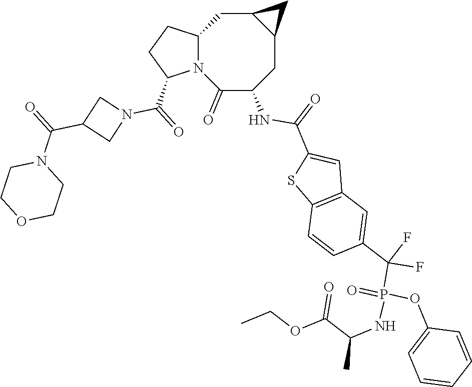 [SMILES: CCOC(=O)[C@H](C)NP(=O)(Oc1ccccc1)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 856.1 [M + H]⁺ | ¹H NMR (400 MHz, CD₃OD) δ 8.23-8.10 (m, 2H), 8.08-7.96 (m, 1H), 7.75-7.53 (m, 1H), 7.38-7.27 (m, 2H), 7.25-7.10 (m, 3H), 5.08-5.00 (m, 1H), 4.77-4.69 (m, 1H), 4.53-4.38 (m, 2H), 4.33-4.17 (m, 2H), 4.11-3.84 (m, 4H), 3.82-3.70 (m, 1H), 3.67-3.44 (m, 6H), 3.38-3.33 (m, 1H), 2.50-2.39 (m, 1H), 2.34-2.07 (m, 4H), 2.04-1.90 (m, 2H), 1.78-1.68 (m, 1H), 1.47-1.35 (m, 1H), 1.48-1.35 (m, 1H), 1.32-1.23 (m, 3H), 1.16 (t, J = 7.1 Hz, 3H), 0.99-0.80 (m, 2H), 0.09-002 (m, 1H). |
| 193 | S-(2- (((difluoro(2- (((3S,6S,7aR, 8aS,9aR)-5- oxo-3-(3- (pyridin-3- yl)azetidine-1- carbonyl) decahydro-1H- cyclopropa[d] pyrrolo[1,2- a]azocin-6- yl)carbamoyl) benzo[b] thiophen-5- yl)methyl) (phenoxy) phosphoryl) oxy)ethyl) 3- methylbutane thioate | 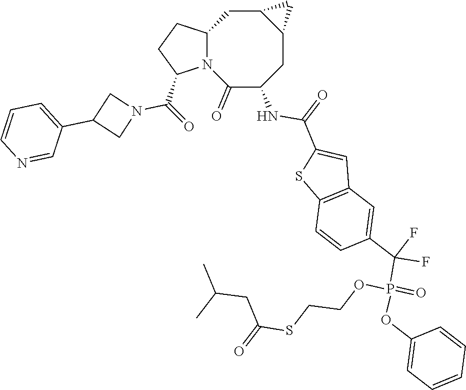 [SMILES: CC(C)CC(=O)SCCOP(=O)(Oc1ccccc1)C(F)(F)c1ccc2sc(C(=O)N[C@H]3C[C@H]4C[C@H]4C[C@H]4CC[C@@H](C(=O)N5CC(c6cccnc6)C5)N4C3=O)cc2c1] | 865.1 [M + H]⁺ | ¹H NMR (400 MHz, CD₃OD) δ 8.59-8.52 (m, 1H), 8.47-8.39 (m, 1H), 8.24-8.16 (m, 2H), 8.12-7.91 (m, 2H), 7.70 (d, J = 8.6 Hz, 1H), 7.50-7.34 (m, 3H), 7.26-7.13 (m, 3H), 5.00-4.94 (m, 1H), 4.73-4.22 (m, 7H), 4.07-3.93 (m, 2H), 3.14 (t, J = 6.2 Hz, 2H), 2.43-2.32 (m, 3H), 2.32-2.12 (m, 3H), 2.09-1.82 (m, 4H), 1.79-1.66 (m, 1H), 1.48-1.40 (m, 1H), 1.35-1.24 (m, 1H), 0.92-0.87 (m, 6H), 0.87-0.81 (m, 1H), 0.22-0.18 (m, 1H). |
| 194 | isopropyl 2- (((difluoro(2- (((3S,6S,7aS, 8aR,9aR)-3-(3- (morpholine-4- carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) (phenoxy) phosphoryl)oxy) acetate | 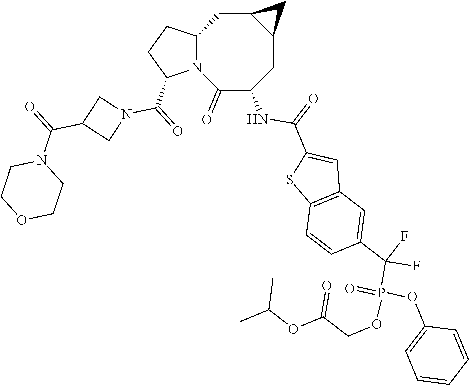 [SMILES: CC(C)OC(=O)COP(=O)(Oc1ccccc1)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 857.1 [M + H]⁺ | ¹H NMR (400 MHz, CD₃OD) δ 8.24-8.14 (m, 2H), 8.10-8.05 (m, 1H), 7.74-7.68 (m, 1H), 7.39-7.32 (m, 2H), 7.26-7.15 (m, 3H), 5.11-4.99 (m, 2H), 4.77-4.65 (m, 3H), 4.62-4.37 (m, 3H), 4.32-4.15 (m, 2H), 4.12-4.02 (m, 1H), 3.82-3.71 (m, 1H), 3.68-3.47 (m, 6H), 3.39-3.35 (m, 1H), 2.50-2.37 (m, 1H), 2.34-2.06 (m, 4H), 2.04-1.91 (m, 2H), 1.79-1.66 (m, 1H), 1.45-1.35 (m, 1H), 1.23-1.21 (m, 6H), 0.99-0.89 (m, 1H), 0.87-0.81 (m, 1H), 0.09-0.03 (m, 1H). |
| 195 | S-(2-((((2- (((3S,6S,7aR, 8aS,9aR)-3- (3-(1H- imidazol-1- yl)azetidine- 1-carbonyl)-5- oxodecahydro- 1H- cyclopropa[d] pyrrolo[1,2- a]azocin-6- yl)carbamoyl) benzo[b] thiophen-5- yl) difluoromethyl) (phenoxy) phosphoryl) oxy)ethyl) 2,2- dimethyl- propanethioate | 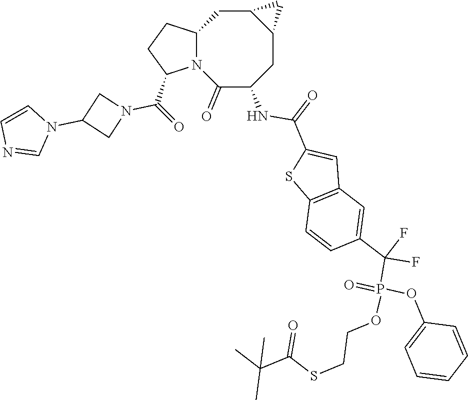 [SMILES: CC(C)(C)C(=O)SCCOP(=O)(Oc1ccccc1)C(F)(F)c1ccc2sc(C(=O)N[C@H]3C[C@H]4C[C@H]4C[C@H]4CC[C@@H](C(=O)N5CC(n6ccnc6)C5)N4C3=O)cc2c1] | 854.3 [M + H]⁺ | ¹H NMR (400 MHz, CD₃OD) δ 8.27-8.20 (m, 1H), 8.20-8.16 (m, 1H), 8.11 (d, J = 8.6 Hz, 1H), 7.99-7.88 (m, 1H), 7.71 (d, J = 8.6 Hz, 1H), 7.59 (br. s., 0.6H), 7.47 (br. s., 0.4H), 7.41-7.32 (m, 2H), 7.28-7.20 (m, 1H), 7.16 (d, J = 8.1 Hz, 2H), 7.09 (br. s., 0.4H), 7.04 (br. s., 0.6H), 5.30-5.20 (m, 1H), 5.06-4.92 (m, 1.6H), 4.82-4.72 (m, 1H), 4.62-4.53 (m, 0.4H), 4.49-4.39 (m, 2H), 4.37-4.10 (m, 4H), 3.11 (t, J = 6.2 Hz, 2H), 2.37 (dd, J = 14.9, 4.6 Hz, 1H), 2.33-2.10 (m, 3H), 2.07-1.82 (m, 3H), 1.78-1.64 (m, 1H), 1.53-1.40 (m, |
| | | | | 1H), 1.36-1.24 (m, |
| | | | | 1H), 1.18-1.11 (m, |
| | | | | 9H), 0.90-0.83 (m, |
| | | | | 1H), 0.23-0.15 (m, 1H). |
| 196 | S-(2-((((2- (((3S,6S,7aR, 8aS,9aR)-3- (3-(1H- imidazol-1- yl)azetidine-1- carbonyl)-5- oxodecahydro- 1H- cyclopropa[d] pyrrolo[1,2- a]azocin-6- yl)carbamoyl) benzo[b] thiophen-5-yl) difluoromethyl) (phenoxy) phosphoryl) oxy)ethyl) 3- methylbutane thioate | 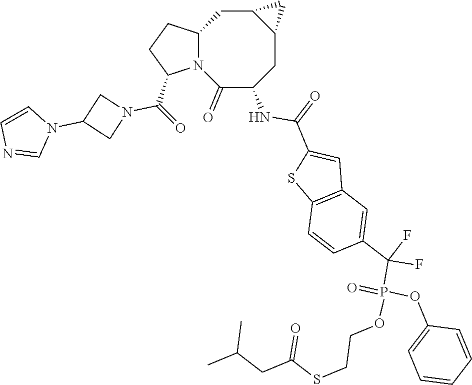 [SMILES: CC(C)CC(=O)SCCOP(=O)(Oc1ccccc1)C(F)(F)c1ccc2sc(C(=O)N[C@H]3C[C@H]4C[C@H]4C[C@H]4CC[C@@H](C(=O)N5CC(n6ccnc6)C5)N4C3=O)cc2c1] | 854.1 [M + H]⁺ | ¹H NMR (400 MHz, CD₃OD) δ 8.26-8.16 (m, 2H), 8.10 (d, J = 8.8 Hz, 1H), 7.90-7.81 (m, 1H), 7.70 (d, J = 8.6 Hz, 1H), 7.58-7.42 (m, 1H), 7.39-7.33 (m, 2H), 7.27-7.12 (m, 3H), 7.09-6.99 (m, 1H), 5.27-5.19 (m, 1H), 5.05-4.89 (m, 2H), 4.81-4.72 (m, 1H), 4.60-4.40 (m, 2H), 4.36-4.11 (m, 4H), 3.13 (t, J = 6.4 Hz, 2H), 2.41-2.33 (m, 3H), 2.32-2.12 (m, 3H), 2.11-1.80 (m, 4H), 1.78-1.65 (m, 1H), 1.55-1.40 (m, 1H), 1.34-1.24 (m, 1H), 0.93-0.81 (m, 7H), 0.21-0.17 (m, 1H). |
| 197 | methyl (2S)- 2-(((difluoro(2- (((3S,6S,7aS, 8aR,9aR)-3-(3- (morpholine-4- carbonyl) azetidine-1- carbonyl)-5- oxodecahydro- 1H-cyclopropa [d]pyrrolo[1,2- a]azocin-6- yl)carbamoyl) benzo[b] thiophen-5- yl)methyl) (phenoxy) phosphoryl)oxy) propanoate | 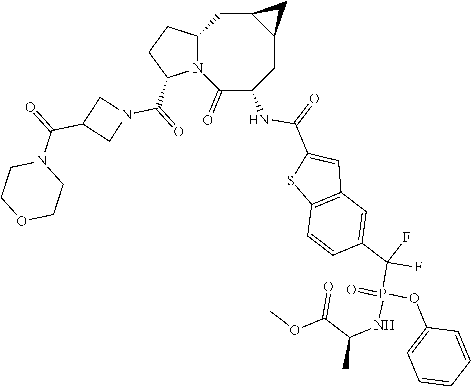 [SMILES: COC(=O)[C@H](C)NP(=O)(Oc1ccccc1)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 843.1 [M + H]⁺ | ¹H NMR (400 MHz, CD₃OD) δ 8.24-8.16 (m, 2H), 8.13-8.04 (m, 1H), 7.73-7.69 (m, 1H), 7.39-7.32 (m, 2H), 7.26-7.10 (m, 3H), 5.12-4.93 (m, 2H), 4.75-4.68 (m, 1H), 4.52-4.38 (m, 2H), 4.33-4.16 (m, 2H), 4.11-4.03 (m, 1H), 3.82-3.72 (m, 1H), 3.68-3.46 (m, 9H), 3.39-3.32 (m, 2H), 2.50-2.38 (m, 1H), 2.35-2.08 (m, 4H), 2.04-1.91 (m, 2H), 1.78-1.66 (m, 1H), 1.52-1.36 (m, 4H), 0.99-0.89 (m, 1H), 0.88-0.83 (m, 1H), 0.08-0.03 (m, 1H). |
| 198 | S-(2-((((2- (((3S,6S,7aS, 8aR,9aR)-3- (3-(1H- imidazol-1- yl)azetidine-1- carbonyl)-5- oxodecahydro- 1H- cyclopropa[d] pyrrolo[1,2- a]azocin-6- yl)carbamoyl) benzo[b] thiophen-5-yl) difluoromethyl) (phenoxy) phosphoryl) oxy)ethyl) butanethioate | 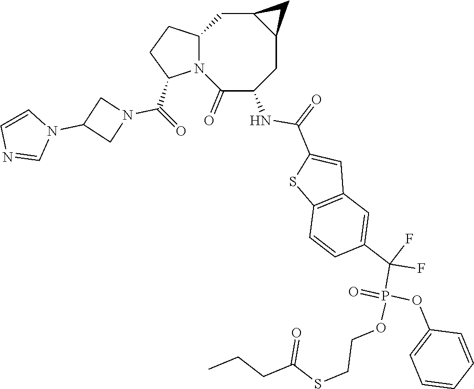 [SMILES: CCCC(=O)SCCOP(=O)(Oc1ccccc1)C(F)(F)c1ccc2sc(C(=O)N[C@H]3C[C@@H]4C[C@@H]4C[C@H]4CC[C@@H](C(=O)N5CC(n6ccnc6)C5)N4C3=O)cc2c1] | 840.1 [M + H]⁺ | ¹H NMR (400 MHz, CD₃OD) δ 8.23-8.18 (m, 2H), 8.12-8.08 (m, 1H), 7.90-7.84 (m, 1H), 7.72-7.66 (m, 1H), 7.56-7.43 (m, 1H), 7.39-7.32 (m, 2H), 7.27-7.20 (m, 1H), 7.17-7.11 (m, 2H), 7.08-7.00 (m, 1H), 5.28-5.18 (m, 1H), 5.11-5.02 (m, 1H), 4.84-4.75 (m, 1H), 4.60-4.41 (m, 3H), 4.35-4.21 (m, 3H), 4.17-4.09 (m, 1H), 3.16-3.10 (m, 2H), 2.55-2.41 (m, 3H), 2.35-2.12 (m, 4H), 2.08-1.93 (m, 2H), 1.81-1.69 (m, 1H), 1.68-1.55 (m, 2H), 1.49-1.36 (m, 1H), 1.01-0.94 (m, |
| | | | | 1H), 0.93-0.82 (m, |
| | | | | 4H), 0.12-0.04 (m, 1H). |
| 199 | methyl 2- (((difluoro(2- (((3S,6S,7aS, 8aR,9aR)-3-(3- (morpholine-4- carbonyl) azetidine-1- carbonyl)-5- oxodecahydro- 1H- cyclopropa[d] pyrrolo[1,2- a]azocin-6- yl)carbamoyl) benzo[b] thiophen-5- yl)methyl) (phenoxy) phosphoryl)oxy) acetate | 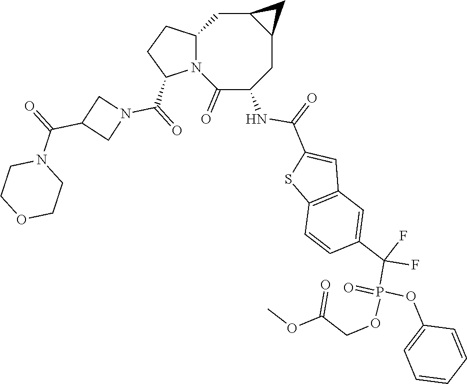 [SMILES: COC(=O)COP(=O)(Oc1ccccc1)C(F)(F)c1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1] | 829.0 [M + H]⁺ | ¹H NMR (400 MHz, CD₃OD) δ 8.24-8.14 (m, 2H), 8.10-8.04 (m, 1H), 7.74-7.68 (m, 1H), 7.39-7.32 (m, 2H), 7.26-7.14 (m, 3H), 5.06-5.01 (m, 1H), 4.80-4.66 (m, 3H), 4.52-4.36 (m, 2H), 4.33-4.17 (m, 2H), 4.12-4.02 (m, 1H), 3.83-3.75 (m, 1H), 3.72 (s, 3H), 3.68- 3.47 (m, 6H), 3.41- 3.32 (m, 2H), 2.51- 2.38 (m, 1H), 2.34- 2.06 (m, 4H), 2.04- 1.91 (m, 2H), 1.79- 1.67 (m, 1H), 1.45- 1.35 (m, 1H), 0.98- 0.89 (m, 1H), 0.87- 0.80 (m, 1H), 0.08- 0.04 (m, 1H). |
| 200 | isopropyl (((2- (((3S,6S,7aS, 8aR,9aR)-3-(3- (morpholine-4- carbonyl) azetidine-1- carbonyl)-5- oxodecahydro- 1H-cyclopropa [d]pyrrolo[1,2- a]azocin-6- yl)carbamoyl) benzo[b] thiophen-5- yl)methyl) (2,2,2- trifluoroethoxy) phosphoryl)-L- alaninate | 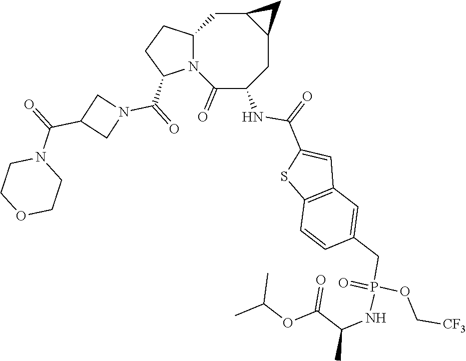 [SMILES: CC(C)OC(=O)[C@H](C)NP(=O)(Cc1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1)OCC(F)(F)F] | 840.1 [M + H]⁺ | ¹H NMR (400 MHz, CD₃CN) δ 7.90-7.77 (m, 3H), 7.71-7.57 (m, 1.5H), 7.55-7.48 (m, 0.5H), 7.42-7.34 (m, 1H), 5.00-4.89 (m, 2H), 4.65-4.55 (m, 1H), 4.47-4.24 (m, 4H), 4.23-4.15 (m, 1H), 4.12-3.92 (m, 2H), 3.90-3.73 (m, 2H), 3.63-3.49 (m, 6H), 3.41-3.27 (m, 2H), 3.27-3.20 (m, 2H), 2.35-2.25 (m, 1H), 2.24-2.17 (m, 1H), 2.11-2.00 (m, 3H), 1.92-1.82 (m, 2H), 1.69-1.57 (m, 1H), 1.48-1.37 (m, 1H), 1.33-1.25 (m, 2.5H), 1.23-1.12 (m, 6.5H), 0.88-0.73 (m, |
| | | | | 2H), 0.03-−0.02 (m, |
| | | | | 1H). |
| 201 | S,S′- ((((difluoro(2- (((3S,6S,7aR, 8aS,9aR)-3- (3-(4-methyl- 4H-1,2,4- triazol-3- yl)azetidine- 1-carbonyl)-5- oxodecahydro- 1H- cyclopropa[d] pyrrolo[1,2- a]azocin-6- yl)carbamoyl) benzo[b] thiophen-5- yl)methyl) phosphoryl) bis(oxy)) bis(ethane-2,1- diyl)) bis(2,2- dimethyl- propanethioate) | 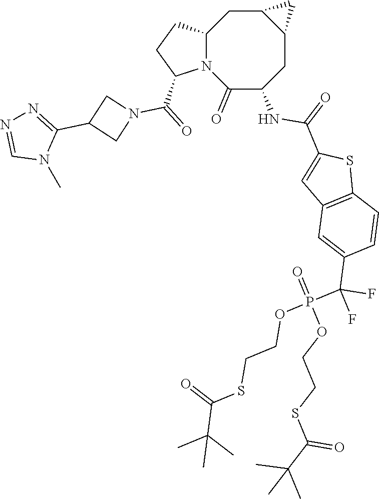 [SMILES: Cn1cnnc1C1CN(C(=O)[C@@H]2CC[C@@H]3C[C@@H]4C[C@@H]4C[C@H](NC(=O)c4cc5cc(C(F)(F)P(=O)(OCCSC(=O)C(C)(C)C)OCCSC(=O)C(C)(C)C)ccc5s4)C(=O)N32)C1] | 937.0 [M + H]⁺ | ¹H NMR (500 MHz, CDCl₃) δ 9.72-9.66 (m, 1H), 8.35 (m, 1H), 8.12 (d, J = 18.1 Hz, 1H), 7.94 (d, J = 8.6 Hz, 1H), 7.92-7.80 (m, 1H), 7.63 (dd, J = 18.4, 8.6 Hz, 1H), 5.00-4.70 (m, 3H), 4.64-4.40 (m, 2H), 4.33 (s, 1H), 4.25-4.06 (m, 6H), 3.84 (s, 3H), 3.10 (m, 4H), 2.44 (m, 2H), 2.21 (m, 1H), 2.13- 1.87 (m, 5H), 1.76- 1.52 (m, 2H), 1.21 (s, 18H), 0.80 (br. s, 1H), 0.17 br. s, 1H). |
| 202 | S,S′- ((((difluoro(2- (((3S,6S,7aR, 8aS,9aR)-3- (3-methoxy- 3-(1-methyl- 1H-imidazol- 2-yl)azetidine- 1-carbonyl)-5- oxodecahydro- 1H- cyclopropa[d] pyrrolo[1,2-a] azocin-6- yl)carbamoyl) benzo[b] thiophen-5- yl)methyl) phosphoryl) bis(oxy)) bis(ethane-2,1- diyl)) bis(2,2- dimethyl- propanethioate) | 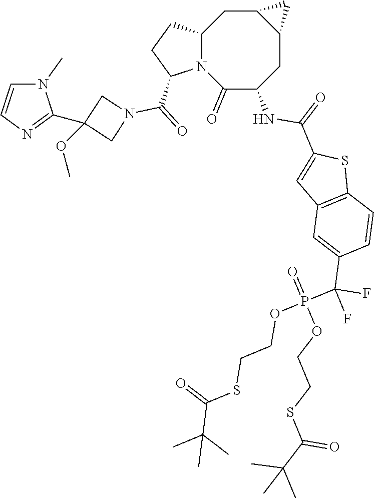 [SMILES: COC1(c2nccn2C)CN(C(=O)[C@@H]2CC[C@@H]3C[C@@H]4C[C@@H]4C[C@H](NC(=O)c4cc5cc(C(F)(F)P(=O)(OCCSC(=O)C(C)(C)C)OCCSC(=O)C(C)(C)C)ccc5s4)C(=O)N32)C1] | 966.0 [M + H]⁺ | ¹H NMR (500 MHz, CDCl₃) δ 8.09 (s, 1H), 7.95-7.77 (m, 3H), 7.65 (d, J = 8.2 Hz, 1H), 7.39 (s, 1H), 7.16 (s, 1H), 4.89 (s, 1H), 4.83 (m, 1H), 4.58- 4.43 (m, 2H), 4.22 (m, 3H), 4.16 (m, 3H), 3.81 (m, 3H), 3.23 (s, 3H), 3.11 (m, 4H), 2.39- 2.28 (m, 1H), 2.09 (m, 2H), 1.92 (m, 4H), 1.73 (m, 4H), 1.21 (s, 18H), 0.80 (m, 1H), 0.17 (m, 1H). |
| 203 | ((((2- (((3S,6S,7aR, 8aS,9aR)-3- (3-(1H- imidazol-4- yl)azetidine- 1-carbonyl)-5- oxodecahydro- 1H-cyclopropa [d]pyrrolo[1,2- a]azocin-6- yl)carbamoyl) benzo[b] thiophen-5-yl) difluoromethyl) phosphoryl) bis(oxy)) bis(methylene) bis(2,2- dimethyl- propanoate) | 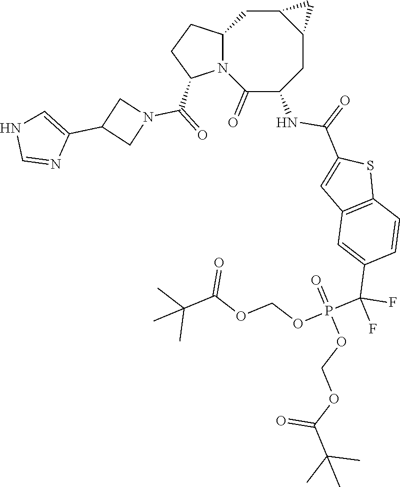 [SMILES: CC(C)(C)C(=O)OCOP(=O)(OCOC(=O)C(C)(C)C)C(F)(F)c1ccc2sc(C(=O)N[C@H]3C[C@H]4C[C@H]4C[C@H]4CC[C@@H](C(=O)N5CC(c6c[nH]cn6)C5)N4C3=O)cc2c1] | 863.0 [M + H]⁺ | |
| 204 | (((difluoro(2- (((3S,6S,7aS, 8aR,9aR)-3-(3- morpholino- azetidine-1- carbonyl)-5- oxodecahydro- 1H- cyclopropa[d] pyrrolo[1,2-a] azocin-6- yl)carbamoyl) benzo[b] thiophen-5- yl)methyl) phosphoryl) bis(oxy)) bis(methylene) bis(2,2- dimethyl- propanoate) | 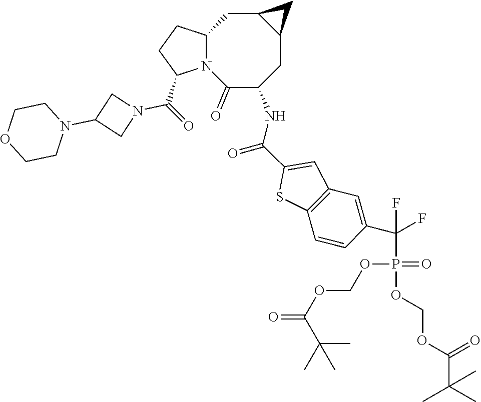 [SMILES: CC(C)(C)C(=O)OCOP(=O)(OCOC(=O)C(C)(C)C)C(F)(F)c1ccc2sc(C(=O)N[C@H]3C[C@@H]4C[C@@H]4C[C@H]4CC[C@@H](C(=O)N5CC(N6CCOCC6)C5)N4C3=O)cc2c1] | 881.4 [M + H]⁺ | ¹H NMR (400 MHz, CDCl₃) δ 8.09 (s, 1H), 7.94 (d, J = 8.4 Hz, 1H), 7.83 (s, 1H), 7.67- 7.60 (m, 1H), 7.48- 7.31 (m, 1H), 5.79- 5.71 (m, 2H), 5.70- 5.63 (m, 2H), 5.14- 4.97 (m, 1H), 4.86- 4.60 (m, 1H), 4.49- 4.12 (m, 4H), 4.00 (s, 4H), 3.45-3.17 (m, 1H), 2.63-2.40 (m, 2H), 2.36-2.25 (m, 3H), 2.18-1.97 (m, 4H), 1.56-1.51 (m, 2H), 1.45 (d, J = 6.8 Hz, 2H), 1.21 (d, J = 1.2 Hz, 19H), 1.02- 0.90 (m, 1H), 0.89- 0.79 (m, 1H), 0.09- −0.08 (m, 1H). |
| 205 | (((difluoro(2- (((3S,6S,7aS, 8aR,9aR)-5- oxo-3-(3- (tetrahydro- 2H-pyran-4- yl)azetidine-1- carbonyl) decahydro-1H- cyclopropa[d] pyrrolo[1,2- a]azocin-6- yl)carbamoyl) benzo[b] thiophen-5- yl)methyl) phosphoryl) bis(oxy)) bis(methylene) bis(2,2- dimethyl- propanoate) | 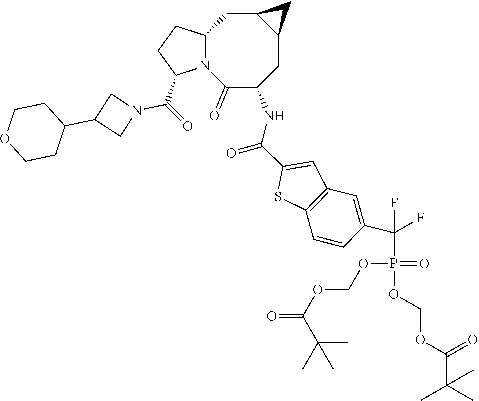 [SMILES: CC(C)(C)C(=O)OCOP(=O)(OCOC(=O)C(C)(C)C)C(F)(F)c1ccc2sc(C(=O)N[C@H]3C[C@@H]4C[C@@H]4C[C@H]4CC[C@@H](C(=O)N5CC(C6CCOCC6)C5)N4C3=O)cc2c1] | 880.4 [M + H]⁺ | ¹H NMR (400 MHz, CD₃OD) δ 8.19 (d, J = 2.4 Hz, 1H), 8.14 (s, 1H), 8.11-8.05 (m, 1H), 7.62 (d, J = 8.8 Hz, 1H), 5.84-5.68 (m, 4H), 5.11-4.99 (m, 1H), 4.58 (s, 1H), 4.53- 4.45 (m, 1H), 4.28 (d, J = 7.6 Hz, 2H), 3.95 (d, J = 11.2 Hz, 3H), 3.77-3.64 (m, 1H), 3.50-3.39 (m, 2H), 2.53-2.37 (m, 2H), 2.19 (s, 4H), 1.95 (d, J = 8.8 Hz, 2H), 1.76 (s, 2H), 1.66-1.57 (m, 2H), 1.43 (d, J = 2.4 Hz, 1H), 1.30 (d, J = 4.0 Hz, 2H), 1.19-1.16 (m, 18H), 0.84 (d, J = 4.4 Hz, 2H), 0.06 (d, J = 5.2 Hz, 1H). |
| 206 | (((difluoro(2- (((3S,6S,7aS, 8aR,9aR)-5- oxo-3-(3- (pyridin-2- yl)azetidine-1- carbonyl) decahydro-1H- cyclopropa[d] pyrrolo[1,2- a]azocin-6- yl)carbamoyl) benzo[b] thiophen-5- yl)methyl) phosphoryl) bis(oxy)) bis(methylene) bis(2,2- dimethyl- propanoate) | 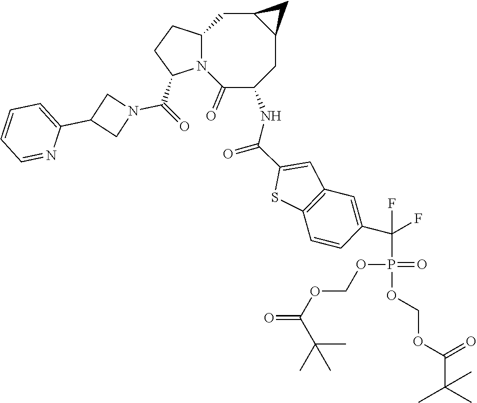 [SMILES: CC(C)(C)C(=O)OCOP(=O)(OCOC(=O)C(C)(C)C)C(F)(F)c1ccc2sc(C(=O)N[C@H]3C[C@@H]4C[C@@H]4C[C@H]4CC[C@@H](C(=O)N5CC(c6ccccn6)C5)N4C3=O)cc2c1] | 873.2 [M + H]⁺ | ¹H NMR (400 MHz, CD₃OD) δ 8.61-8.44 (m, 1H), 8.18-8.12 (m, 1H), 8.11-8.01 (m, 1H), 7.80 (m, 1H), 7.65-7.58 (m, 1H), 7.53-7.37 (m, 1H), 7.36-7.23 (m, 2H), 5.81-5.67 (m, 4H), 5.09-4.89 (m, 2H), 4.81-4.64 (m, 1H), 4.61-4.54 (m, 1H), 4.49-4.42 (m, 1H), 4.42-4.26 (m, 1H), 4.24-4.00 (m, 2H), 2.54-2.37 (m, 1H), 2.36-2.26 (m, 2H), 2.25-2.12 (m, 2H), 2.11-1.88 (m, 2H), 1.80-1.67 (m, 1H), 1.50-1.38 (m, 1H), 1.20-1.18 (m, 18H), 1.03-0.90 (m, 1H), 0.89-0.81 (m, 1H), 0.06 (m, 1H). |
| 207 | (((difluoro(2- (((3S,6S,7aS, 8aR,9aR)-5- oxo-3-(3- (pyridin-2-yl) azetidine-1- carbonyl) decahydro-1H- cyclopropa[d] pyrrolo[1,2- a]azocin-6- yl)carbamoyl) benzo[b] thiophen-5- yl)methyl) phosphoryl) bis(oxy)) bis(methylene) bis(2,2- dimethyl- propanoate) | 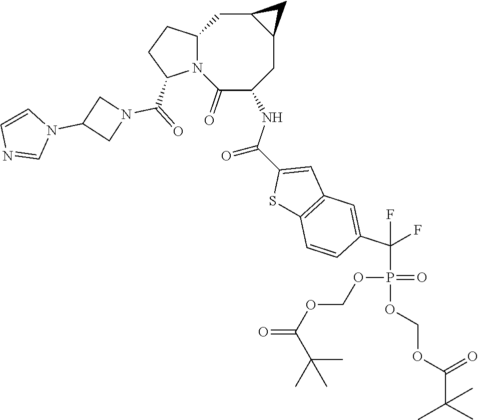 [SMILES: CC(C)(C)C(=O)OCOP(=O)(OCOC(=O)C(C)(C)C)C(F)(F)c1ccc2sc(C(=O)N[C@H]3C[C@@H]4C[C@@H]4C[C@H]4CC[C@@H](C(=O)N5CC(n6ccnc6)C5)N4C3=O)cc2c1] | 862.4 [M + H]⁺ | ¹H NMR (400 MHz, CDCl₃) δ 8.34 (d, J = 1.2 Hz, 1H), 8.18-7.93 (m, 2H), 7.92-7.83 (m, 1H), 7.75 (d, J = 5.6 Hz, 1H), 7.69-7.59 (m, 1H), 7.36 (s, 1H), 7.25-7.14 (m, 1H), 5.82-5.59 (m, 4H), 5.30-5.00 (m, 3H), 4.80-4.62 (m, 1H), 4.50-4.32 (m, 2H), 4.23 (d, J = 5.6 Hz, 1H), 4.13 (d, J = 7.2 Hz, 1H), 2.44 (s, 1H), 2.35 (s, 2H), 2.16 (d, J = 5.6 Hz, 1H), 2.12- 2.05 (m, 2H), 1.69 (d, J = 2.8 Hz, 1H), 1.43 (s, 1H), 1.30-1.26 (m, 2H), 1.25-1.20 (m, 18H), 0.98-0.92 (m, 1H), 0.89-0.83 (m, 1H), 0.03 (d, J = 4.8 Hz, 1H). |
| 208 | ((((2- (((3S,6S,7aS, 8aR,9aR)-3- (((3S,4S)-1- acetyl-4- hydroxy- pyrrolidin-3- yl)carbamoyl) 5-oxodecahydro- 1H- cyclopropa[d] pyrrolo[1,2- a]azocin-6- yl)carbamoyl) benzo[b] thiophen-5-yl) difluoromethyl) phosphoryl) bis(oxy)) bis(methylene) bis(2,2- dimethyl- propanoate) | 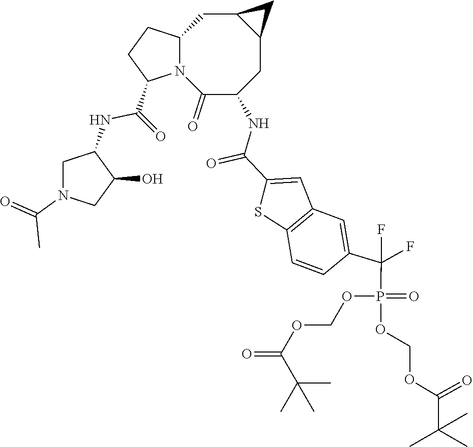 [SMILES: CC(=O)N1C[C@H](NC(=O)[C@@H]2CC[C@@H]3C[C@H]4C[C@H]4C[C@H](NC(=O)c4cc5cc(C(F)(F)P(=O)(OCOC(=O)C(C)(C)C)OCOC(=O)C(C)(C)C)ccc5s4)C(=O)N32)[C@@H](O)C1] | 883.2 [M + H]⁺ | ¹H NMR (400 MHz, CD₃OD) δ 8.19-8.04 (m, 3H), 7.62 (d, J = 8.8 Hz, 1H), 5.83-5.64 (m, 4H), 5.04 (m, 1H), 4.60-4.49 (m, 1H), 4.31-4.07 (m, 3H), 3.90-3.53 (m, 2H), 3.46-3.35 (m, 2H), 2.52-2.40 (m, 1H), 2.33-2.25 (m, 1H), 2.23-2.10 (m, 3H), 2.05 (d, J = 10.4 Hz, 3H), 2.01-1.94 (m, 1H), 1.79-1.65 (m, 1H), 1.22 (s, 2H), 1.18 (s, 18H), 0.89-0.86 (m, 2H), 0.14-0.05 (m, 1H). |
| 209 | (((difluoro(2- (((3S,6S,7aR, 8aS,9aR)-5- oxo-3-(3- (pyridin-2- yl)azetidine-1- carbonyl) decahydro-1H- cyclopropa[d] pyrrolo[1,2- a]azocin-6- yl)carbamoyl) benzo[b] thiophen-5- yl)methyl) phosphoryl) bis(oxy)) bis(methylene) bis(2,2- dimethyl- propanoate) | 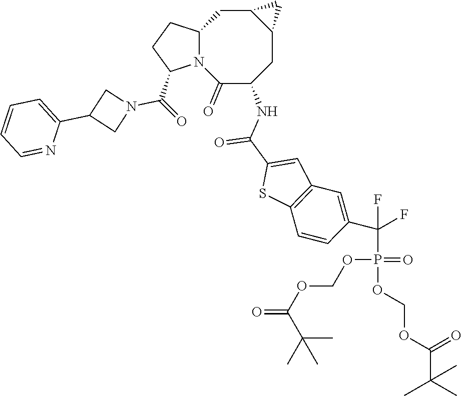 [SMILES: CC(C)(C)C(=O)OCOP(=O)(OCOC(=O)C(C)(C)C)C(F)(F)c1ccc2sc(C(=O)N[C@H]3C[C@H]4C[C@H]4C[C@H]4CC[C@@H](C(=O)N5CC(c6ccccn6)C5)N4C3=O)cc2c1] | 873.2 [M + H]⁺ | ¹H NMR (400 MHz, CDCl₃) δ 8.69-8.58 (m, 1H), 8.09 (s, 1H), 7.98-7.91 (m, 1H), 7.87-7.81 (m, 2H), 7.77-7.62 (m, 2H), 7.26-7.18 (m, 2H), 5.74 (dd, J = 4.8, 12.4 Hz, 2H), 5.70-5.62 (m, 2H), 4.94-4.74 (m, 2H), 4.58-4.35 (m, 3H), 4.25-4.14 (m, 2H), 4.12-4.01 (m, 1H), 2.37-2.29 (m, 1H), 2.28-2.20 (m, 1H), 2.14-2.05 (m, 3H), 2.04-1.92 (m, 2H), 1.90-1.78 (m, 1H), 1.27-1.23 (m, 1H), 1.21 (s, 18H), 1.19 (s, 2H), 0.82 (m, 1H), 0.22 (m, 1H). |
| 210 | (((difluoro(2- (((3S,6S,7aS, 8aR,9aR)-5-oxo- 3-(6-(2-phenyl- acetyl)-2,6- diazaspiro[3.3] heptane-2- carbonyl) decahydro-1H- cyclopropa[d] pyrrolo[1,2- a]azocin-6- yl)carbamoyl) benzo[b] thiophen-5- yl)methyl) phosphoryl) bis(oxy)) bis(methylene) bis(2,2- dimethyl- propanoate) | 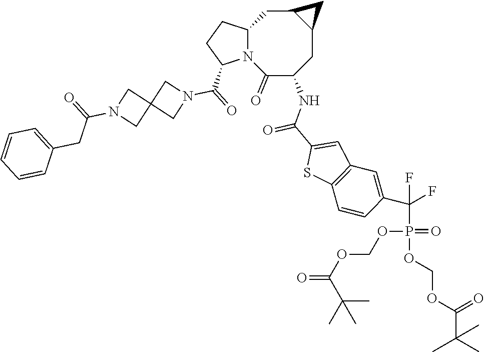 [SMILES: CC(C)(C)C(=O)OCOP(=O)(OCOC(=O)C(C)(C)C)C(F)(F)c1ccc2sc(C(=O)N[C@H]3C[C@@H]4C[C@@H]4C[C@H]4CC[C@@H](C(=O)N5CC6(CN(C(=O)Cc7ccccc7)C6)C5)N4C3=O)cc2c1] | 955 [M + H]⁺ | ¹H NMR (400 MHz, CD₃CN) δ 8.57-8.34 (m, 1H), 8.03 (s, 1H), 7.95-7.86 (m, 2H), 7.55 (d, J = 8.4 Hz, 1H), 7.35-7.08 (m, 5H), 5.79-5.57 (m, 4H), 5.13-4.97 (m, 1H), 4.59 (d, J = 8.8 Hz, 1H), 4.45 (dd, J = 3.6, 7.6 Hz, 1H), 4.34- 4.12 (m, 4H), 4.06- 3.85 (m, 4H), 3.39- 3.27 (m, 2H), 2.39- 2.30 (m, 1H), 2.14 (dd, J = 5.6, 12.0 Hz, 4H), 1.92-1.83 (m, 2H), 1.74-1.58 (m, 1H), 1.48-1.33 (m, 1H), 1.16 (d, J = 2.0 Hz, 18H), 0.86-0.70 (m, 2H), 0.00 (d, J = 5.6 Hz, 1H). |
| 211 | ((((2- (((3S,6S,7aS, 8aR,9aR)-3- (4-(1H- pyrazole-4- carbonyl) piperazine-1- carbonyl)-5- oxodecahydro- 1H-cyclopropa[d] pyrrolo[1,2-a] azocin-6- yl)carbamoyl) benzo[b] thiophen-5-yl) difluoromethyl) phosphoryl) bis(oxy)) bis(methylene) bis(2,2- dimethyl- propanoate) | 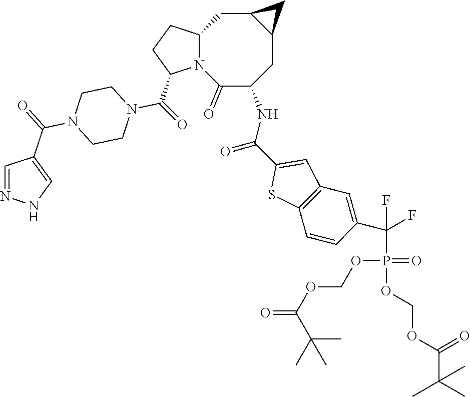 [SMILES: CC(C)(C)C(=O)OCOP(=O)(OCOC(=O)C(C)(C)C)C(F)(F)c1ccc2sc(C(=O)N[C@H]3C[C@@H]4C[C@@H]4C[C@H]4CC[C@@H](C(=O)N5CCN(C(=O)c6cn[nH]c6)CC5)N4C3=O)cc2c1] | 919.4 [M + H]⁺ | ¹H NMR (400 MHz, CD₃CN) δ 8.07 (s, 2H), 8.00-7.92 (m, 2H), 7.75 (s, 1H), 7.57 (d, J = 8.4 Hz, 1H), 5.71- 5.60 (m, 4H), 5.01 (q, J = 7.6 Hz, 1H), 4.93- 4.84 (m, 1H), 4.23 (d, 4.8 Hz, 1H), 3.79- 3.43 (m, 8H), 2.39- 2.30 (m, 2H), 2.10 (d, J = 7.6 Hz, 2H), 1.92- 1.82 (m, 2H), 1.67 (m, 1H), 1.52-1.40 (m, 1H), 1.31 (s, 1H), 1.15 (s, 18H), 0.93-0.84 (m, 1H), 0.80 (m, 1H), 0.01 (m, 1H). |
| 212 | (((difluoro(2- (((3S,6S,7aS, 8aR,9aR)-5- oxo-3-(((R)- 1-(pyridin-3- yl)pyrrolidin-3- yl)carbamoyl) decahydro-1H- cyclopropa[d] pyrrolo[1,2- a]azocin-6- yl)carbamoyl) benzo[b] thiophen-5- yl)methyl) phosphoryl) bis(oxy)) bis(methylene) bis(2,2- dimethyl- propanoate) | 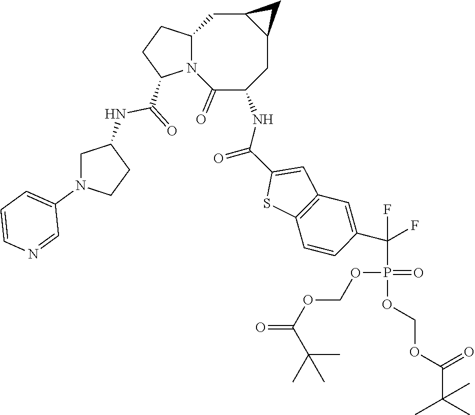 [SMILES: CC(C)(C)C(=O)OCOP(=O)(OCOC(=O)C(C)(C)C)C(F)(F)c1ccc2sc(C(=O)N[C@H]3C[C@@H]4C[C@@H]4C[C@H]4CC[C@@H](C(=O)N[C@@H]5CCN(c6cccnc6)C5)N4C3=O)cc2c1] | 902.1 [M + H]⁺ | ¹H NMR (400 MHz, CDCl₃) δ 8.09 (s, 1H), 7.94 (d, J = 8.0 Hz, 3H), 7.86-7.80 (m, 2H), 7.64 (d, J = 8.4 Hz, 1H), 7.15 (d, J = 6.8 Hz, 2H), 6.80-6.74 (m, 1H), 5.74 (dd, J = 5.2, 12.8 Hz, 2H), 5.66 (dd, J = 4.8, 12.4 Hz, 2H), 5.06 (m, 1H), 4.73 (d, J = 7.6 Hz, 1H), 4.58-4.48 (m, 1H), 4.20-4.09 (m, 1H), 3.53-3.44 (m, 2H), 3.40 (m, 1H), 3.10 (dd, J = 2.4, 10.0 Hz, 1H), 2.53 (dd, J = 5.6, 11.6 Hz, 1H), 2.38-2.27 (m, 1H), 2.25-2.16 (m, 3H), 2.13-2.01 (m, 4H), 1.88-1.75 (m 5H), 1.21 (s, 18H). |
| 213 | (((difluoro(2- (((3S,6S,7aR, 8aS,9aR)-5- oxo-3-(((R)- 1-(pyridin-3- yl)pyrrolidin-3- yl)carbamoyl) decahydro-1H- cyclopropa[d] pyrrolo[1,2- a]azocin-6- yl)carbamoyl) benzo[b] thiophen-5- yl)methyl) phosphoryl) bis(oxy)) bis(methylene) bis(2,2- dimethyl- propanoate) | 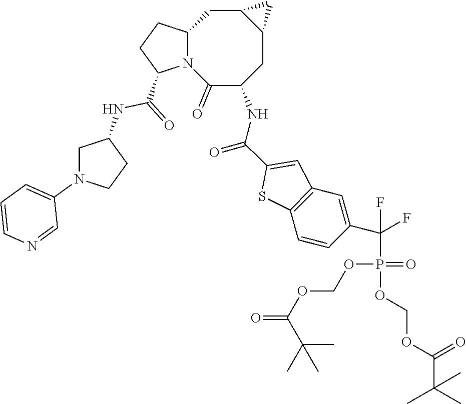 [SMILES: CC(C)(C)C(=O)OCOP(=O)(OCOC(=O)C(C)(C)C)C(F)(F)c1ccc2sc(C(=O)N[C@H]3C[C@H]4C[C@H]4C[C@H]4CC[C@@H](C(=O)N[C@@H]5CCN(c6cccnc6)C5)N4C3=O)cc2c1] | 902.1 [M + H]⁺ | ¹H NMR (400 MHz, CDCl₃) δ 8.13-8.07 (m, 1H), 8.01-7.93 (m, 3H), 7.87-7.82 (m, 1H), 7.72-7.61 (m, 2H), 7.12 (dd, J = 4.4, 8.4 Hz, 1H), 6.88-6.77 (m, 2H), 5.74 (dd, J = 5.2, 12.8 Hz, 2H), 5.66 (dd, J = 4.8, 12.4 Hz, 2H), 4.88 (m, 1H), 4.66-4.59 (m, 1H), 4.50-4.43 (m, 1H), 4.16-4.07 (m, 1H), 3.58 (dd, J = 6.0, 10.4 Hz, 1H), 3.52-3.44 (m, 1H), 3.43-3.35 (m, 1H), 3.33-3.23 (m, 1H), 2.33 (m, 1H), 2.23- 2.17 (m, 3H), 2.11- 1.94 (m, 4H), 1.50- 1.38 (m, 4H), 1.27- 1.24 (m, 3H), 1.21 (s, 18H). |
| 214 | ((((2- (((3S,6S,7aR, 8aS,9aR)-3- (3-(1H- imidazol-1- yl)azetidine-1- carbonyl)-5- oxodecahydro- 1H-cyclopropa[d] pyrrolo[1,2-a] azocin-6- yl)carbamoyl) benzo[b] thiophen-5-yl) difluoromethyl) phosphoryl) bis(oxy)) bis(methylene) bis(2,2- dimethyl- propanoate) | 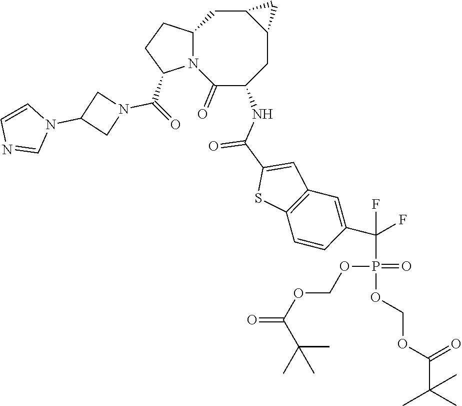 [SMILES: CC(C)(C)C(=O)OCOP(=O)(OCOC(=O)C(C)(C)C)C(F)(F)c1ccc2sc(C(=O)N[C@H]3C[C@H]4C[C@H]4C[C@H]4CC[C@@H](C(=O)N5CC(n6ccnc6)C5)N4C3=O)cc2c1] | 862.4 [M + H]⁺ | ¹H NMR (400 MHz, CD₃OD) δ 8.21-8.07 (m, 1H), 8.01-7.93 (m, 1H), 7.92-7.83 (m, 1H), 7.79-7.61 (m, 3H), 7.22-7.13 (m, 1H), 5.76-5.59 (m, 4H), 5.22-4.99 (m, 2H), 4.98-4.86 (m, 2H), 4.80-4.66 (m, 1H), 4.40-4.29 (m, 2H), 4.24-4.13 (m, 2H), 2.42-2.28 (m, 1H), 2.23-1.86 (m, 7H), 1.32-1.24 (m, 1H), 1.21 (S, 18H), 0.94-0.80 (m, 1H), 0.31-0.15 (m, 1H). |
| 215 | (((difluoro(2- (((3S,6S,7aR, 8aS,9aR)-5- oxo-3-((1-(4,4,4- trifluoro- butanoyl) azetidin-3- yl)carbamoyl) decahydro-1H- cyclopropa[d] pyrrolo[1,2- a]azocin-6- yl)carbamoyl) benzo[b] thiophen-5- yl)methyl) phosphoryl) bis(oxy)) bis(methylene) bis(2,2- dimethyl- propanoate) | 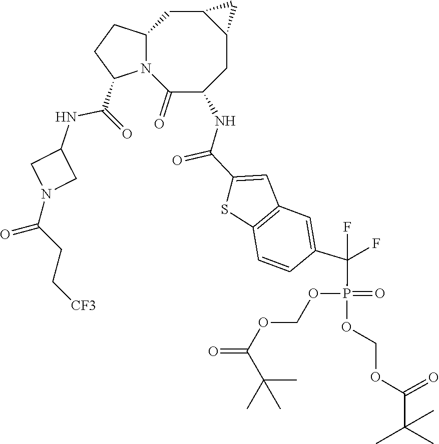 [SMILES: CC(C)(C)C(=O)OCOP(=O)(OCOC(=O)C(C)(C)C)C(F)(F)c1ccc2sc(C(=O)N[C@H]3C[C@H]4C[C@H]4C[C@H]4CC[C@@H](C(=O)NC5CN(C(=O)CCC(F)(F)F)C5)N4C3=O)cc2c1] | 935.3 [M + H]⁺ | ¹H NMR (400 MHz, CDCl₃) δ 8.09 (s, 1H), 7.97-7.86 (m, 2H), 7.75 (s, 1H), 7.63 (d, J = 8.8 Hz, 1H), 7.07- 6.96 (m, 1H), 5.80- 5.60 (m, 4H), 4.93 (s, 1H), 4.74-4.65 (m, 1H), 4.48-4.27 (m, 3H), 4.17 (d, J = 5.2 Hz, 1H), 4.07-3.99 (m, 1H), 3.98-3.91 (m, 1H), 2.45 (s, 2H), 2.38- 2.30 (m, 3H), 2.21- 2.12 (m, 3H), 1.97- 1.89 (m, 1H), 1.87- 1.76 (m, 1H), 1.70- 1.58 (m, 4H), 1.20 (s, 18H), 0.84 (s, 1H), 0.20-0.14 (m, 1H). |
| 216 | methyl (((2- (((3S,6S,7aS, 8aR,9aR)-3-(3- (morpholine-4- carbonyl) azetidine-1- carbonyl)-5- oxodecahydro- 1H-cyclopropa [d]pyrrolo[1,2- a]azocin-6- yl)carbamoyl) benzo[b] thiophen-5- yl)methyl) (phenoxy) phosphoryl)-L- alaninate | 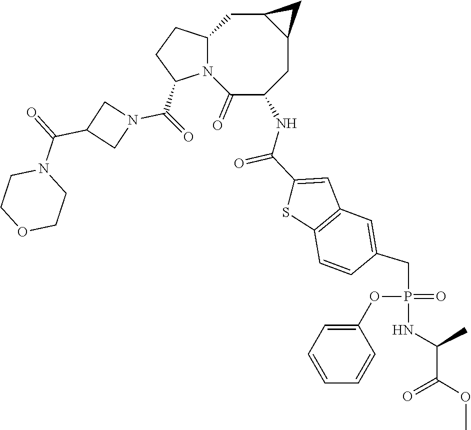 [SMILES: COC(=O)[C@H](C)NP(=O)(Cc1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1)Oc1ccccc1] | 806.4 [M + H]⁺ | ¹H NMR (400 MHz, CDCl₃) δ 7.87-7.77 (m, 3H), 7.54-7.44 (m, 1H), 7.41 (d, J = 7.6 Hz, 1H), 7.33-7.29 (m, 2H), 7.18-7.10 (m, 3H), 5.12-5.01 (m, 1H), 4.77 (t, J = 7.6 Hz, 1H), 4.58-4.51 (m, 1H), 4.44 (d, J = 4.8 Hz, 1H), 4.36-4.29 (m, 1H), 4.23-4.16 (m, 1H), 4.13-4.06 (m, 1H), 4.03-3.94 (m, 1H), 3.71-3.66 (m, 4H), 3.63 (s, 2H), 3.58 (s, 3H), 3.50-3.40 (m, 2H), 3.30 (s, 2H), 2.36- 2.28 (m, 3H), 2.17- 2.09 (m, 2H), 2.06- 2.02 (m, 2H), 1.97- 1.91 (m, 2H), 1.72- 1.64 (m, 1H), 1.49- 1.40 (m, 1H), 1.20- 1.13 (m, 3H), 1.01- 0.92 (m, 1H), 0.87- 0.79 (m, 1H), 0.06- |
| | | | | 0.05 (m, 1H). |
| 217 | propyl (((2- (((3S,6S,7aS, 8aR,9aR)-3-(3- (morpholine-4- carbonyl) azetidine-1- carbonyl)-5- oxodecahydro- 1H-cyclopropa[d] pyrrolo[1,2- a]azocin-6- yl)carbamoyl) benzo[b] thiophen-5- yl)methyl) (phenoxy) phosphoryl)-L- alaninate | 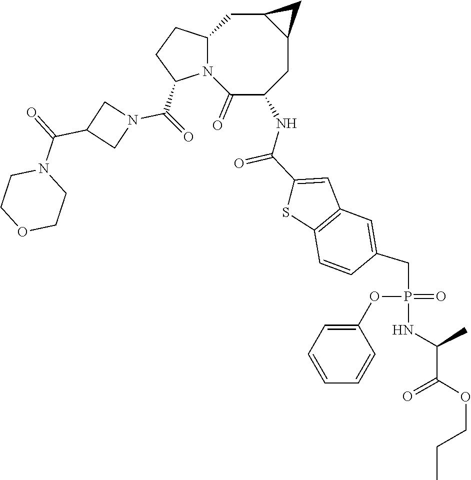 [SMILES: CCCOC(=O)[C@H](C)NP(=O)(Cc1ccc2sc(C(=O)N[C@H]3C[C@@H]4C[C@@H]4C[C@H]4CC[C@@H](C(=O)N5CC(C(=O)N6CCOCC6)C5)N4C3=O)cc2c1)Oc1ccccc1] | 834.7 [M + H]⁺ | ¹H NMR (400 MHz, CDCl₃) δ 7.87-7.74 (m, 3H), 7.50-7.39 (m, 1H), 7.36-7.27 (m, 3H), 7.19-7.10 (m, 3H), 5.05 (m, 1H), 4.78 (m, 1H), 4.59-4.51 (m, 1H), 4.48-4.39 (m, 1H), 4.37-4.28 (m, 1H), 4.21-4.14 (m, 1H), 4.09 (m, 1H), 4.05-3.90 (m, 3H), 3.73-3.58 (m, 7H), 3.50-3.39 (m, 2H), 3.33-3.26 (m, 2H), 3.16-3.06 (m, 1H), 2.37-2.24 (m, 3H), 2.18-1.99 (m, 4H), 1.62-1.52 (m, 3H), 1.47-1.43 (m, 1H), 1.16 (dd, J = 7.2, 10.2 Hz, 3H), 1.01-0.81 (m, 5H), 0.06-0.04 (m, 1H). |
| 218 | S,S′- ((((difluoro(2- (((3S,6S,7aR, 8aS,9aR)-5- oxo-3-(3- (pyridin-2- yl)azetidine-1- carbonyl) decahydro-1H- cyclopropa[d] pyrrolo[1,2-a] azocin-6-yl) carbamoyl)benzo [b]thiophen-5- yl)methyl) phosphoryl) bis(oxy)) bis(ethane-2,1- diyl)) bis(2,2- dimethyl- propanethioate) | 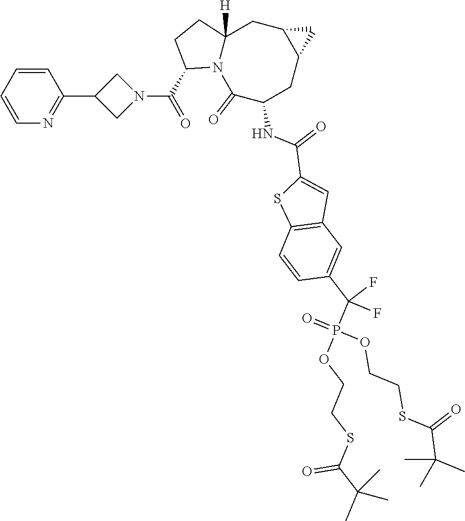 [SMILES: CC(C)(C)C(=O)SCCOP(=O)(OCCSC(=O)C(C)(C)C)C(F)(F)c1ccc2sc(C(=O)N[C@H]3C[C@H]4C[C@H]4C[C@H]4CC[C@@H](C(=O)N5CC(c6ccccn6)C5)N4C3=O)cc2c1] | 933.5 [M + H]⁺ | ¹H NMR (400 MHz, CD₃CN) δ 8.67-8.32 (m, 2H), 8.15-7.86 (m, 3H), 7.71-7.52 (m, 2H), 7.30-7.06 (m, 2H), 4.85 (q, J = 5.8 Hz, 1H), 4.76-4.60 (m, 1H), 4.57-4.29 (m, 3H), 4.26-4.13 (m, 6H), 4.02-3.91 (m, 1H), 3.17-3.04 (m, 4H), 2.29-2.18 (m, 2H), 2.11-2.03 (m, 2H), 2.02-1.98 (m, 1H), 1.91-1.77 (m, 2H), 1.62-1.52 (m, 1H), 1.38-1.26 (m, 2H), 1.19-1.14 (m, 18H), 0.74 (m, 1H), 0.11 (m, 1H). |
| 219 | S,S′-(((((2- (((3S,6S,7aR, 8aS,9aR)-3- (3-(1H- imidazol-4- yl)azetidine- 1-carbonyl)-5- oxodecahydro- 1H- cyclopropa[d] pyrrolo[1,2- a]azocin-6- yl)carbamoyl) benzo[b] thiophen-5-yl) difluoromethyl) phosphoryl) bis(oxy)) bis(ethane-2,1- diyl)) bis(2,2- dimethyl- propanethioate) | 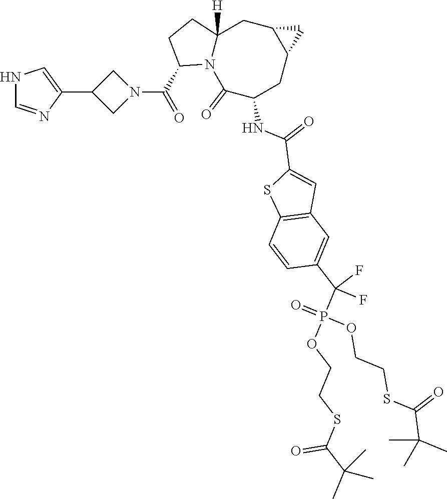 [SMILES: CC(C)(C)C(=O)SCCOP(=O)(OCCSC(=O)C(C)(C)C)C(F)(F)c1ccc2sc(C(=O)N[C@H]3C[C@H]4C[C@H]4C[C@H]4CC[C@@H](C(=O)N5CC(c6c[nH]cn6)C5)N4C3=O)cc2c1] | 922.3 [M + H]⁺ | ¹H NMR (400 MHz, CD₃CN) δ 8.14 (s, 1H), 8.09-7.99 (m, 2H), 7.94 (m, 1H), 7.69-7.61 (m, 2H), 6.97 (d, J = 12.4 Hz, 1H), 4.85 (m, 1H), 4.68-4.45 (m, 1H), 4.40 (d, J = 8.4 Hz, 1H), 4.28-4.05 (m, 7H), 3.98-3.82 (m, 2H), 3.13-3.07 (m, 4H), 2.28-2.16 (m, 2H), 2.13-2.00 (m, 2H), 1.93-1.83 (m, 2H), 1.82-1.72 (m, 1H), 1.70-1.59 (m, 1H), 1.55-1.43 (m, 1H), 1.29-1.22 (m, 1H), 1.18 (d, J = 3.2 Hz, 18H), 0.78 (m, 1H), 0.14-0.08 (m, 1H). |
| 220 | S,S′- ((((difluoro(2- (((3S,6S,7aR, 8aS,9aR)-5- oxo-3-(4- (tetrahydro- 2H-pyran-4- yl)piperazine-1- carbonyl) decahydro-1H- cyclopropa[d] pyrrolo[1,2- a]azocin-6- yl)carbamoyl) benzo[b] thiophen-5- yl)methyl) phosphoryl) bis(oxy)) bis(ethane-2,1- diyl)) bis(2,2- dimethyl- propanethioate) | 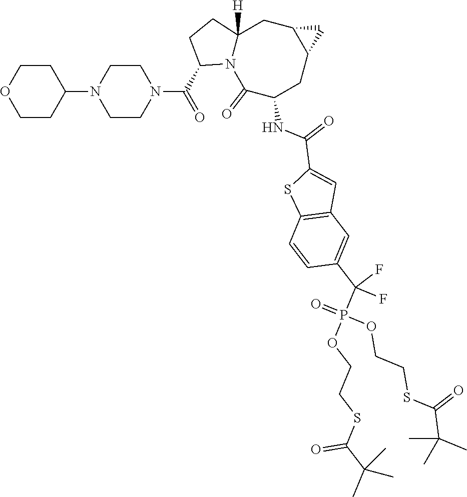 [SMILES: CC(C)(C)C(=O)SCCOP(=O)(OCCSC(=O)C(C)(C)C)C(F)(F)c1ccc2sc(C(=O)N[C@H]3C[C@H]4C[C@H]4C[C@H]4CC[C@@H](C(=O)N5CCN(C6CCOCC6)CC5)N4C3=O)cc2c1] | 969.7 [M + H]⁺ | ¹H NMR (400 MHz, CD₃OD) δ 8.19-8.13 (m, 2H), 8.04 (d, J = 8.4 Hz, 1H), 7.64 (d, J = 8.3 Hz, 1H), 4.97 (d, J = 4.6 Hz, 1H), 4.59 (s, 1H), 4.35 (t, J = 5.6 Hz, 1H), 4.29- 4.14 (m, 4H), 3.95 (d, J = 1.6 Hz, 2H), 3.81-3.49 (m, 4H), 3.40-3.34 (m, 2H), 3.21-3.10 (m, 4H), 2.82-2.72 (m, 1H), 2.64 (s, 4H), 2.38- 2.27 (m, 2H), 2.27- 2.17 (m, 1H), 2.16- 2.02 (m, 2H), 1.97- 1.93 (m, 1H), 1.87- 1.72 (m, 4H), 1.57- 1.40 (m, 3H), 1.24- 1.18 (m, 18H), 0.88- 0.84 (m, 1H), 0.26- 0.18 (m, 1H). |
| 221 | S,S′-(((((7- (((3S,6S,7aS, 8aR,9aR)-3- (3-(1H- imidazol-1- yl)azetidine-1- carbonyl)-5- oxodecahydro- 1H- cyclopropa[d] pyrrolo[1,2- a]azocin-6- yl)carbamoyl) naphthalen-2-yl) difluoromethyl) phosphoryl) bis(oxy)) bis(ethane-2,1- diyl)) bis(2,2- dimethyl- propanethioate) | 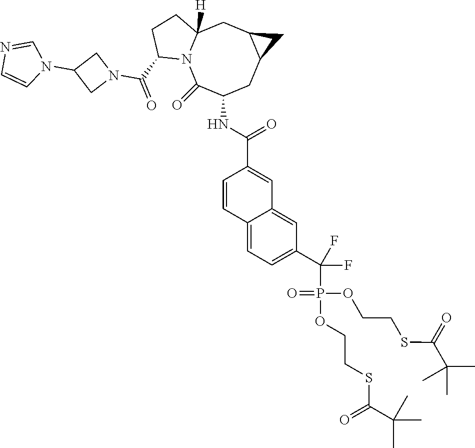 [SMILES: CC(C)(C)C(=O)SCCOP(=O)(OCCSC(=O)C(C)(C)C)C(F)(F)c1ccc2ccc(C(=O)N[C@H]3C[C@@H]4C[C@@H]4C[C@H]4CC[C@@H](C(=O)N5CC(n6ccnc6)C5)N4C3=O)cc2c1] | 916.3 [M + H]⁺ | ¹H NMR (400 MHz, CD₃OD) δ 8.60 (s, 1H), 8.29 (s, 1H), 8.10 (d, J = 8.4 Hz, 1H), 8.05 (s, 2H), 7.87 (d, J = 4.0 Hz, 1H), 7.77 (d, J = 8.4 Hz, 1H), 7.57-7.44 (m, 1H), 7.05 (d, J = 17.2 Hz, 1H), 5.27- 5.19 (m, 1H), 5.12 (d, J = 8.4 Hz, 1H), 4.82- 4.76 (m, 1H), 4.60- 4.52 (m, 2H), 4.51- 4.41 (m, 1H), 4.35 (d, J = 6.4 Hz, 1H), 4.29- 4.11 (m, 5H), 3.19- 3.08 (m, 4H), 2.49 (m, 1H), 2.36-2.13 (m, 4H), 2.08-1.94 (m, 2H), 1.85-1.72 (m, 1H), 1.47-1.37 (m, 1H), 1.18 (d, J = 3.2 Hz, 18H), 1.03-0.95 (m, 1H), 0.86 (m, 1H), 0.09 (m, 1H). |
| 222 | S,S′- ((((difluoro(2- (((3S,6S,7aR, 8aS,9aR)-5- oxo-3-(3- (pyrimidin-5- yl)azetidine-1- carbonyl) decahydro-1H- cyclopropa[d] pyrrolo[1,2- a]azocin-6- yl)carbamoyl) benzo[b] thiophen-5- yl)methyl) phosphoryl) bis(oxy)) bis(ethane-2,1- diyl)) bis(3- methylbutane thioate) | 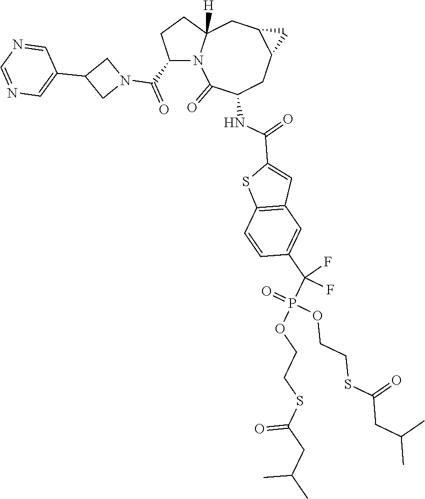 [SMILES: CC(C)CC(=O)SCCOP(=O)(OCCSC(=O)CC(C)C)C(F)(F)c1ccc2sc(C(=O)N[C@H]3C[C@H]4C[C@H]4C[C@H]4CC[C@@H](C(=O)N5CC(c6cncnc6)C5)N4C3=O)cc2c1] | 934.3 [M + H]⁺ | ¹H NMR (400 MHz, CDCl₃) δ 8.76 (d, J = 4.9 Hz, 1H), 8.17-8.06 (m, 1H), 8.01-7.93 (m, 1H), 7.91-7.79 (m, 2H), 7.74-7.65 (m, 1H), 7.24 (m, 1H), 4.98-4.83 (m, 2H), 4.64-4.56 (m, 1H), 4.55-4.40 (m, 2H), 4.30-4.14 (m, 6H), 3.20-2.98 (m, 4H), 2.19 (m, 3H), 2.13- 2.06 (m, 4H), 1.33-1.22 (m, 8H), 1.00-0.88 (m, 12H). |
| 223 | S,S′-(((((2- (((3S,6S,7aR, 8aS,9aR)-5- oxo-3-(3- (pyridin-3- yl)azetidine-1- carbonyl) decahydro-1H- cyclopropa[d] pyrrolo[1,2- a]azocin-6- yl)carbamoyl) benzo[b] thiophen-5- yl)methyl) phosphoryl) bis(oxy)) bis(ethane-2,1- diyl)) bis(2,2- dimethyl- propanethioate) | 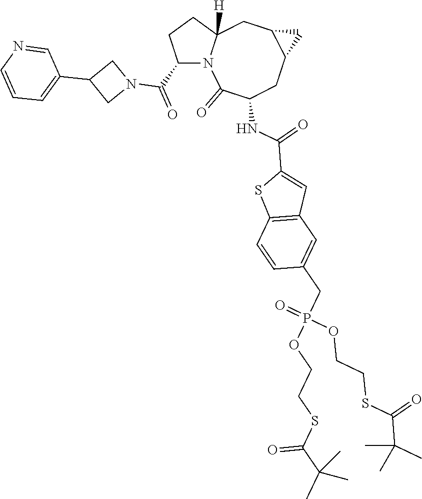 [SMILES: CC(C)(C)C(=O)SCCOP(=O)(Cc1ccc2sc(C(=O)N[C@H]3C[C@H]4C[C@H]4C[C@H]4CC[C@@H](C(=O)N5CC(c6cccnc6)C5)N4C3=O)cc2c1)OCCSC(=O)C(C)(C)C] | 897.3 [M + H]⁺ | ¹H NMR (400 MHz, DMSO-d₆) δ 8.86- 8.76 (m, 1H), 8.56 (s, 1H), 8.49-8.44 (m, 1H), 8.21 (d, J = 4.4 Hz, 1H), 7.95 (d, J = 8.4 Hz, 1H), 7.86 (s, 1H), 7.44-7.33 (m, 2H), 4.86-4.81 (m, 1H), 4.63-4.59 (m, 1H), 4.42-4.15 (m, 4H), 4.02-3.91 (m, 5H), 3.87-3.84 (m, 1H), 3.49-3.38 (m, 2H), 3.17-2.93 (m, 5H), 2.29-2.15 (m, 2H), 2.12-1.98 (m, 2H), 1.97-1.83 (m, 2H), 1.75-1.63 (m, 1H), 1.60-1.47 (m, 1H), 1.37-1.26 (m, 1H), 1.16 (d, J = 2.8 Hz, 18H), 0.80-0.75 (m, 1H), 0.07-0.00 (m, 1H). |
| 224 | S,S′- ((((difluoro(2- (((3S,6S,7aR, 8aS,9aR)-5- oxo-3-(((R)- 1-(pyridin-3- yl)pyrrolidin-3- yl)carbamoyl) decahydro-1H- cyclopropa[d] pyrrolo[1,2- a]azocin-6- yl)carbamoyl) benzo[b] thiophen-5- yl)methyl) phosphoryl) bis(oxy)) bis(ethane-2,1- diyl)) bis(3- methylbutane thioate) | 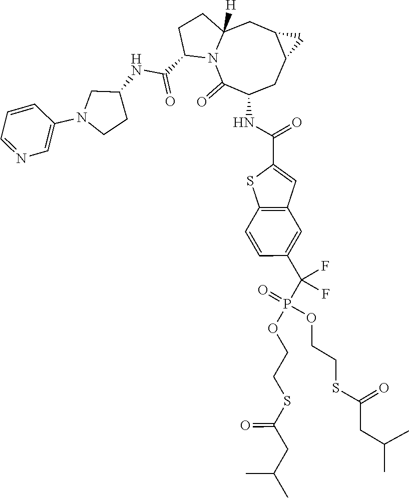 [SMILES: CC(C)CC(=O)SCCOP(=O)(OCCSC(=O)CC(C)C)C(F)(F)c1ccc2sc(C(=O)N[C@H]3C[C@H]4C[C@H]4C[C@H]4CC[C@@H](C(=O)N[C@@H]5CCN(c6cccnc6)C5)N4C3=O)cc2c1] | 962.2 [M + H]⁺ | ¹H NMR (400 MHz, CD₃OD) δ 8.19 (s, 1H), 8.15-8.08 (m, 2H), 8.00-7.91 (m, 2H), 7.79-7.73 (m, 1H), 7.72-7.63 (m, 2H), 4.53 (s, 1H), 4.31 (d, J = 5.6 Hz, 1H), 4.24- 4.13 (m, 4H), 3.72- 3.66 (m, 1H), 3.61- 3.54 (m, 1H), 3.53- 3.47 (m, 1H), 3.16- 2.99 (m, 7H), 2.41- 2.32 (m, 2H), 2.30- 2.22 (m, 2H), 2.17 (d, J = 6.8 Hz, 4H), 2.13 (d, J = 5.6 Hz, 1H), 2.04-2.01 (m, 2H), 1.80-1.70 (m, 2H), 1.65-1.53 (m, 3H), 0.92 (d, J = 6.4 Hz, 12H), 0.87 (s, 3H), 0.23-0.14 (m, 1H). |
| 225 | S,S′- ((((difluoro(2- (((3S,6S,7aR, 8aS,9aR)-5- oxo-3-(3- (pyrimidin-2- yl)azetidine-1- carbonyl) decahydro-1H- cyclopropa[d] pyrrolo[1,2- a]azocin-6- yl)carbamoyl) benzo[b] thiophen-5- yl)methyl) phosphoryl) bis(oxy)) bis(ethane-2,1- diyl)) bis(3- methylbutane thioate) | 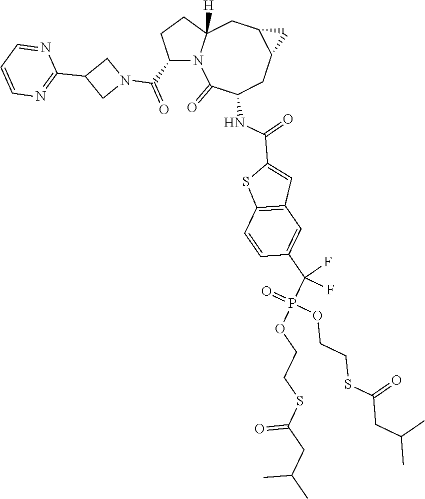 [SMILES: CC(C)CC(=O)SCCOP(=O)(OCCSC(=O)CC(C)C)C(F)(F)c1ccc2sc(C(=O)N[C@H]3C[C@H]4C[C@H]4C[C@H]4CC[C@@H](C(=O)N5CC(c6ncccn6)C5)N4C3=O)cc2c1] | 934.3 [M + H]⁺ | ¹H NMR (400 MHz, CDCl₃) δ 8.76 (d, J = 4.9 Hz, 1H), 8.17-8.06 (m, 1H), 8.01-7.93 (m, 1H), 7.91-7.79 (m, 2H), 7.74-7.65 (m, 1H), 7.24 (m, 1H), 4.98-4.83 (m, 2H), 4.64-4.56 (m, 1H), 4.55-4.40 (m, 2H), 4.30-4.14 (m, 6H), 3.20-2.98 (m, 4H), 2.19 (m, 3H), 2.13- 2.06 (m, 4H), 1.33- 1.22 (m, 8H), 1.00-0.88 (m, 12H). |
| 226 | diisopropyl (((((2- (((3S,6S,7aR, 8aS,9aR)-5- oxo-3-(((R)- 1-(pyridin-3- yl)pyrrolidin-3- yl)carbamoyl) decahydro-1H- cyclopropa[d] pyrrolo[1,2- a]azocin-6- yl)carbamoyl) benzo[b] thiophen-5- yl)methyl) phosphoryl) bis(oxy)) bis(methylene)) bis(carbonate) | 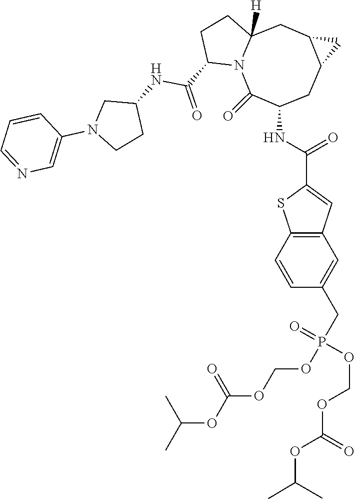 [SMILES: CC(C)OC(=O)OCOP(=O)(Cc1ccc2sc(C(=O)N[C@H]3C[C@H]4C[C@H]4C[C@H]4CC[C@@H](C(=O)N[C@@H]5CCN(c6cccnc6)C5)N4C3=O)cc2c1)OCOC(=O)OC(C)C] | 870.2 [M + H]⁺ | ¹H NMR (400 MHz, CDCl₃) δ 8.05-8.00 (m, 1H), 7.91-7.85 (m, 1H), 7.82-7.76 (m, 3H), 7.72 (s, 1H), 7.35 (m, 1H), 7.07-6.93 (m, 1H), 5.66-5.57 (m, 4H), 4.92-4.88 (m, 2H), 4.59 (m, 2H), 4.16 (s, 1H), 3.74-3.63 (m, 1H), 3.62-3.53 (m, 1H), 3.44-3.35 (m, 2H), 3.34-3.26 (m, 2H), 2.35-2.23 (m, 2H), 2.20-2.13 (m, 3H), 1.99-1.88 (m, 2H), 1.83-1.76 (m, 4H), 1.33-1.28 (m, 12H), 1.25-1.12 (m, 2H), 0.85-0.74 (m, 1H), 0.13-0.04 (m, 1H). |
| 227 | S,S′-(((((2- (((3S,6S,7aS, 8aR,9aR)-3- (3-(4- cyanophenyl)-3- fluoroazetidine- 1-carbonyl)-5- oxodecahydro- 1H-cyclopropa[d] pyrrolo[1,2-a] azocin-6- yl)carbamoyl) benzo[b] thiophen-5-yl) difluoromethyl) phosphoryl) bis(oxy)) bis(ethane-2,1- diyl)) bis(2,2- dimethyl- propanethioate) | 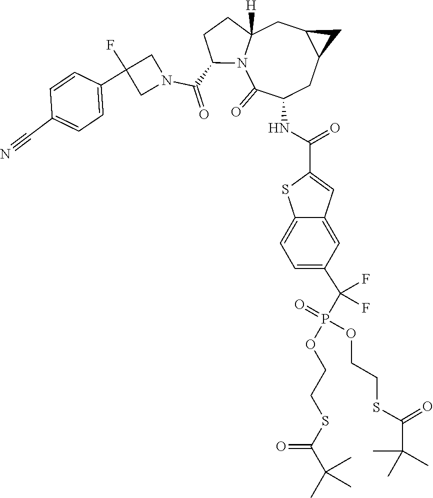 [SMILES: CC(C)(C)C(=O)SCCOP(=O)(OCCSC(=O)C(C)(C)C)C(F)(F)c1ccc2sc(C(=O)N[C@H]3C[C@@H]4C[C@@H]4C[C@H]4CC[C@@H](C(=O)N5CC(F)(c6ccc(C#N)cc6)C5)N4C3=O)cc2c1] | 975.6 [M + H]⁺ | ¹H NMR (400 MHz, CD₃OD) δ 8.20-8.14 (m, 2H), 8.06 (d, J = 8.4 Hz, 1H), 7.83 (d, J = 8.4 Hz, 1H), 7.79- 7.71 (m, 3H), 7.65 (d, J = 8.4 Hz, 1H), 5.17- 5.02 (m, 2H), 4.81- 4.65 (m, 1H), 4.61- 4.53 (m, 1H), 4.41 (s, 1H), 4.38-4.29 (m, 2H), 4.28-4.13 (m, 4H), 3.22-3.08 (m, 4H), 2.47 (m, 1H), 2.37-2.27 (m, 2H), 2.26-2.11 (m, 2H), 2.09-1.91 (m, 2H), 1.75 (m, 1H), 1.48- 1.36 (m, 1H), 1.22- 1.15 (m, 18H) 1.05- 0.92 (m, 1H), 0.89- 0.80 (m, 1H), 0.08 (m, 1H). |
| 228 | S,S′- ((((difluoro(2- (((3S,6S,7aS, 8aR,9aR)-3- (3-fluoro-3- phenylazetidine- 1-carbonyl)-5- oxodecahydro- 1H-cyclopropa[d] pyrrolo[1,2- a]azocin-6- yl)carbamoyl) benzo[b] thiophen-5- yl)methyl) phosphoryl) bis(oxy)) bis(ethane-2,1- diyl)) bis(2,2- dimethyl- propanethioate) | 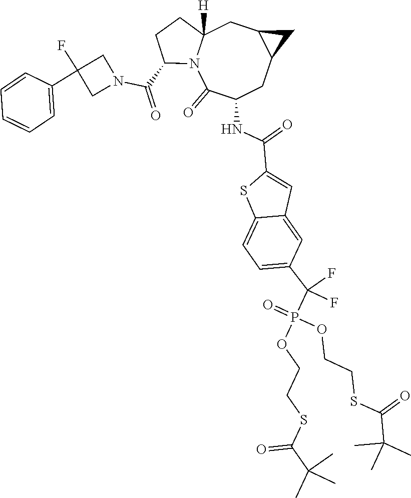 [SMILES: CC(C)(C)C(=O)SCCOP(=O)(OCCSC(=O)C(C)(C)C)C(F)(F)c1ccc2sc(C(=O)N[C@H]3C[C@@H]4C[C@@H]4C[C@H]4CC[C@@H](C(=O)N5CC(F)(c6ccccc6)C5)N4C3=O)cc2c1] | 950.2 [M + H]⁺ | ¹H NMR (400 MHz, CDCl₃) δ 8.07 (s, 1H), 7.91 (d, J = 8.4 Hz, 1H), 7.86-7.78 (m, 1H), 7.62 (d, J = 8.8 Hz, 1H), 7.53 (d, J = 7.2 Hz, 1H), 7.47- 7.36 (m, 4H), 5.22- 4.96 (m, 2H), 4.66- 4.54 (m, 1H), 4.52- 4.40 (m, 2H), 4.39- 4.26 (m, 1H), 4.25- 4.07 (m, 5H), 3.17- 2.99 (m, 4H), 2.40- 2.23 (m, 3H), 2.17- 2.00 (m, 4H), 1.68- 1.62 (m, 1H), 1.45- 1.42 (m, 1H), 1.19 (s, 18H), 1.01-0.92 (m, 1H), 0.86-0.79 (m, 1H), −0.01 (d, J = 4.8 Hz, 1H). |
| 229 | S,S′- ((((difluoro(2- (((3S,6S,7aS, 8aR,9aR)-5- oxo-3-(3- (pyrimidin-2- yl)azetidine-1- carbonyl) decahydro-1H- cyclopropa[d] pyrrolo[1,2-a] azocin-6-yl) carbamoyl) benzo[b] thiophen-5- yl)methyl) phosphoryl) bis(oxy)) bis(ethane-2,1- diyl)) bis(3- methylbutane thioate) | 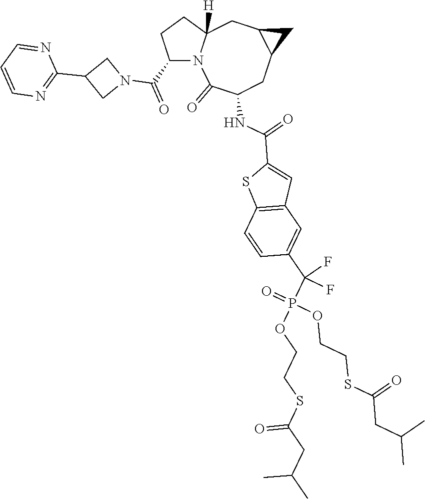 [SMILES: CC(C)CC(=O)SCCOP(=O)(OCCSC(=O)CC(C)C)C(F)(F)c1ccc2sc(C(=O)N[C@H]3C[C@@H]4C[C@@H]4C[C@H]4CC[C@@H](C(=O)N5CC(c6ncccn6)C5)N4C3=O)cc2c1] | 934.3 [M + H]⁺ | ¹H NMR (400 MHz, CDCl₃) δ 8.90-8.78 (m, 2H), 8.17-8.10 (m, 1H), 7.99-7.93 (m, 1H), 7.90-7.84 (m, 1H), 7.70 (d, J = 7.6 Hz, 1H), 7.51-7.39 (m, 1H), 7.37-7.28 (m, 1H), 5.11-4.90 (m, 2H), 4.67-4.55 (m, 2H), 4.51 (d, J = 6.4 Hz, 1H), 4.47-4.36 (m, 1H), 4.29-4.17 (m, 6H), 3.18-2.98 (m, 4H), 2.38-2.25 (m, 3H), 2.21-2.17 (m, 4H), 2.12 (s, 1H), 1.73-1.64 (m, 1H), 1.52-1.45 (m, 1H), 1.33-1.22 (m, 3H), 1.05-0.98 (m, 1H), 0.95 (d, J = 6.8 Hz, 12H), 0.91-0.79 (m, 3H), 0.05-0.02 (m, 1H) |

Synthesis of (3S,6S,8aR,9aS,9bR)-6-((tert-butoxycarbonyl)amino)-8a-methyl-5-oxodecahydro-1H-cyclopropa[c]pyrrolo[1,2-a]azocine-3-carboxylic acid

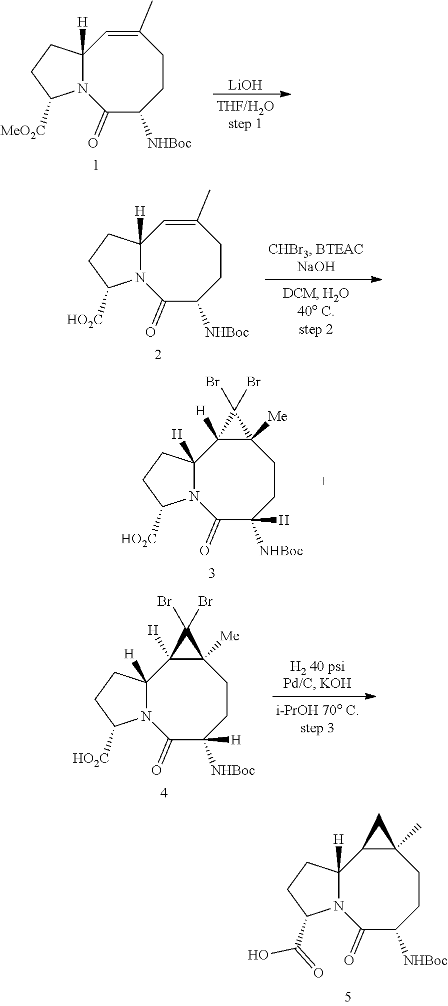

Step 1: (3S,6S,10aR)-6-((tert-butoxycarbonyl)amino)-9-methyl-5-oxo-1,2,3,5,6,7,8,10a-octahydropyrrolo[1,2-a]azocine-3-carboxylic acid

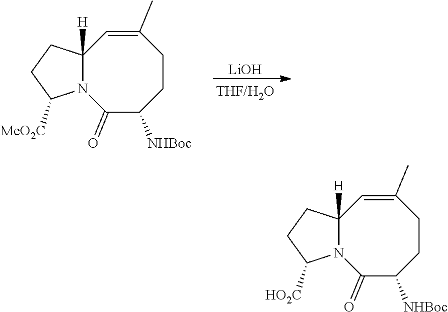 [SMILES: COC(=O)[C@@H]1CC[C@@H]2/C=C(/C)CC[C@H](NC(=O)OC(C)(C)C)C(=O)N21 ; "LiOH" ; C/C1=C/[C@H]2CC[C@@H](C(=O)O)N2C(=O)[C@@H](NC(=O)OC(C)(C)C)CC1]

To a solution of (3S,6S,10aR)-methyl 6-((tert-butoxycarbonyl)amino)-9-methyl-5-oxo-1,2,3,5,6,7,8,10a-octahydropyrrolo[1,2-a]azocine-3-carboxylate (1.5 g, 4.25 mmol, 1 eq) in tetrahydrofuran (6 mL) and water (2 mL) was added lithium hydroxide monohydrate (356 mg, 8.50 mmol, 2.0 eq). The reaction was stirred for 16 h at room temperature. The reaction was then partially concentrated under reduced pressure to remove THF. The reaction was diluted with water (20 mL) and EtOAc (50 mL). The phases were separated and the aqueous phase was acidified with 1N aqueous HCl to pH=1-2. The product was then extracted with DCM (3×50 mL). The combined organic layers were dried with sodium sulfate, filtered and concentrated under reduced pressure to give (3S,6S,10aR)-6-((tert-butoxycarbonyl)amino)-9-methyl-5-oxo-1,2,3,5,6,7,8,10a-octahydropyrrolo[1,2-a]azocine-3-carboxylic acid (1.43 g, 100%) as a white solid. LCMS (ESI) m/z=339.2 (M+H)⁺.

Step 2: (3S,6S,8aR,9aR,9bR)-9,9-dibromo-6-((tert-butoxycarbonyl)amino)-8a-methyl-5-oxodecahydro-1H-cyclopropa[c]pyrrolo[1,2-a]azocine-3-carboxylic acid and (3S,6S,8aS,9aS,9bR)-9,9-dibromo-6-((tert-butoxycarbonyl)amino)-8a-methyl-5-oxodecahydro-1H-cyclopropa[c]pyrrolo[1,2-a]azocine-3-carboxylic acid

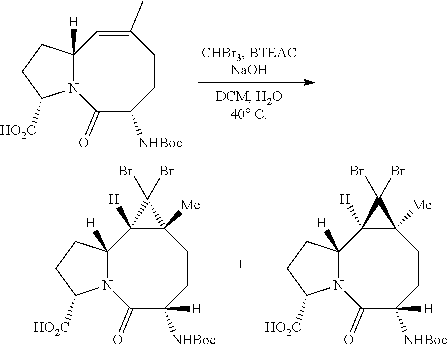

To a solution of (3S,6S,10aR)-6-((tert-butoxycarbonyl)amino)-9-methyl-5-oxo-1,2,3,5,6,7,8,10a-octahydropyrrolo[1,2-a]azocine-3-carboxylic acid (1.45 g, 4.28 mmol, 1 eq) in methylene chloride (15 mL) were added bromoform (5.22 mL, 59.9 mmol, 14 eq) and benzyltriethylammonium chloride (309 mg, 1.36 mmol, 0.32 eq) under nitrogen atmosphere. A solution of sodium hydroxide (3.76 g, 94.1 mmol, 22 eq) in water (7.52 mL) was added. The reaction was heated at reflux for 72 h. The reaction was then concentrated under reduced pressure to remove DCM, water and most of excess bromoform. The resulting dark brown residue was dissolved in water (50 mL) and acidified with 3 N aqueous HCl to pH 1-2. The aqueous layer was extracted with DCM (3×50 mL) and the combined organic layers were dried with sodium sulfate, filtered and concentrated under reduced pressure. The crude residue was purified by reverse phase chromatography on a 275 g C₁₈ cartridge eluting with 5-60% MeCN in water (with 0.1% formic acid). The first eluting product (3S,6S,8aS,9aS,9bR)-9,9-dibromo-6-((tert-butoxycarbonyl)amino)-8a-methyl-5-oxodecahydro-1H-cyclopropa[c]pyrrolo[1,2-a]azocine-3-carboxylic acid (479 mg, 21.9%) was isolated as a white solid. LCMS (ESI) m/z=408.9 (M-Boc+H)⁺; ¹H NMR (400 MHz, CDCl₃) δ 5.87 (d, J=6.8 Hz, 1H), 4.54 (t, J=8.8 Hz, 1H), 4.30-4.22 (m, 1H), 3.96-3.88 (m, 1H), 2.52-1.96 (m, 8H), 1.69 (d, J=10.8 Hz, 1H), 1.44 (s, 9H), 1.36 (s, 3H).

The second eluting product (3S,6S,8aR,9aR,9bR)-9,9-dibromo-6-((tert-butoxycarbonyl)amino)-8a-methyl-5-oxodecahydro-1H-cyclopropa[c]pyrrolo[1,2-a]azocine-3-carboxylic acid (191 mg, 8.8%) was isolated as a white solid. LCMS (ESI) m/z=408.9 (M-Boc+H)⁺; ¹H NMR (400 MHz, CDCl₃) δ 5.91 (s, 1H), 5.36 (d, J=8.8 Hz, 1H), 4.78-4.70 (m, 1H), 4.66 (t, J=7.6 Hz, 1H), 3.39-3.28 (m, 2H), 2.89 (dt, J=13.9, 7.2 Hz, 2H), 2.48-2.38 (m, 1H), 2.34-2.24 (m, 1H), 2.05-1.95 (m, 1H), 1.87 (s, 3H), 1.84-1.75 (m, 1H), 1.61-1.52 (m, 1H), 1.44 (s, 9H).

Step 3: (3S,6S,8aR,9aS,9bR)-6-((tert-butoxycarbonyl)amino)-8a-methyl-5-oxodecahydro-1H-cyclopropa[c]pyrrolo[1,2-a]azocine-3-carboxylic acid

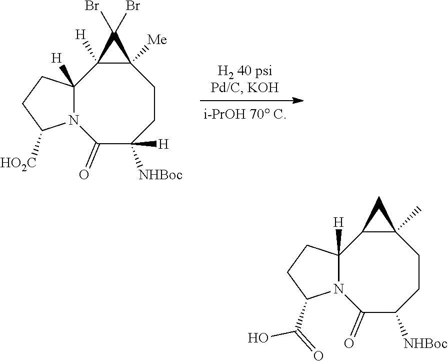 [SMILES: CC(C)(C)OC(=O)N[C@H]1CC[C@@]2(C)[C@@H]([C@H]3CC[C@@H](C(=O)O)N3C1=O)C2(Br)Br ; CC(C)(C)OC(=O)N[C@H]1CC[C@]2(C)C[C@@H]2[C@H]2CC[C@@H](C(=O)O)N2C1=O]

In a tube, (3S,6S,8aS,9aS,9bR)-9,9-dibromo-6-((tert-butoxycarbonyl)amino)-8a-methyl-5-oxodecahydro-1H-cyclopropa[c]pyrrolo[1,2-a]azocine-3-carboxylic acid (600 mg, 1.17 mmol, 1 eq) was dissolved in 2-propanol (10 mL) under nitrogen atmosphere. Then, potassium hydroxide (393 mg, 7.02 mmol, 6 eq) and 10% palladium on carbon (50% wet) (400 mg, 188 μmol, 0.16 eq) were added. The tube was filled with hydrogen (40 psi) and sealed. The reaction mixture was heated to 70° C. for 20 h. The reaction mixture was then cooled down to room temperature, diluted with MeOH and filtered on a Celite pad. The filtrate was concentrated under reduced pressure. The crude residue was purified by reverse phase chromatography on a 50 g C₁₈ cartridge eluting with 5-60% MeCN in water (with 0.1% formic acid) to afford after lyophilization (3S,6S,8aR,9aS,9bR)-6-((tert-butoxycarbonyl)amino)-8a-methyl-5-oxodecahydro-1H-cyclopropa[c]pyrrolo[1,2-a]azocine-3-carboxylic acid (120 mg, 29.1%) as a white solid. LCMS (ESI) m/z=353.2 (M+H)⁺; ¹H NMR (400 MHz, CDCl₃) δ 5.87 (d, J=6.8 Hz, 1H), 4.56 (t, J=8.6 Hz, 1H), 4.24 (t, J=7.9 Hz, 1H), 3.63 (dd, J=11.0, 6.4 Hz, 1H), 2.68-2.51 (m, 1H), 2.38-2.20 (m, 2H), 2.14-1.87 (m, 4H), 1.53 (dd, J=15.3, 10.6 Hz, 1H), 1.44 (s, 9H), 1.04-0.93 (m, 4H), 0.54 (dd, J=8.6, 4.9 Hz, 1H), 0.33 (t, J=5.0 Hz, 1H).

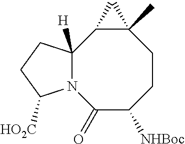 [SMILES: CC(C)(C)OC(=O)N[C@H]1CC[C@@]2(C)C[C@H]2[C@H]2CC[C@@H](C(=O)O)N2C1=O]

(3S,6S,8aS,9aR,9bR)-6-((tert-butoxycarbonyl)amino)-8a-methyl-5-oxodecahydro-1H-cyclopropa[c]pyrrolo[1,2-a]azocine-3-carboxylic acid was synthesized under same conditions as above. LCMS (ESI) m/z=353.2 (M+H)⁺.

Synthesis of [(2-{[(1R,3S,5R,7S,10S)-3-methyl-8-oxo-10-[3-(pyridin-3-yl)azetidine-1-carbonyl]-9-azatricyclo[7.3.0.0^{3,5}]dodecan-7-yl]carbamoyl}-1-benzothiophen-5-yl)methyl]phosphonic acid

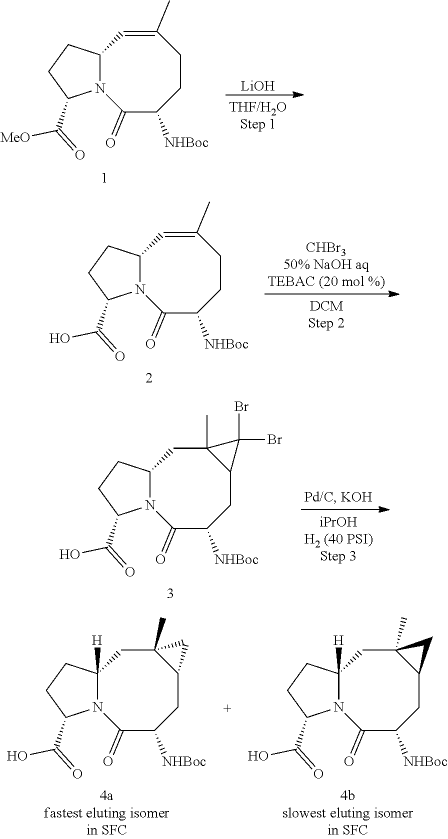

Step 1: (3S,6S,10aR)-6-{[(tert-butoxy)carbonyl]amino}-9-methyl-5-oxo-1H,2H,3H,5H,6H,7H,10H,10aH-pyrrolo[1,2-a]azocine-3-carboxylic acid

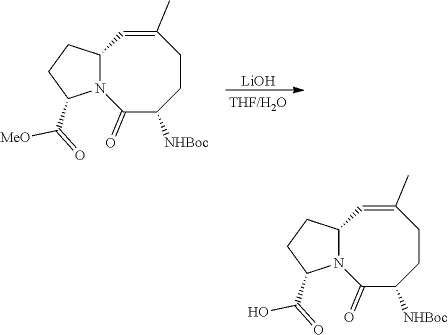 [SMILES: COC(=O)[C@@H]1CC[C@@H]2/C=C(/C)CC[C@H](NC(=O)OC(C)(C)C)C(=O)N21 ; "LiOH" ; C/C1=C/[C@H]2CC[C@@H](C(=O)O)N2C(=O)[C@@H](NC(=O)OC(C)(C)C)CC1]

To a solution of methyl (3S,6S,10aR)-6-{[(tert-butoxy)carbonyl]amino}-9-methyl-5-oxo-1H,2H,3H,5H,6H,7H,10H,10aH-pyrrolo[1,2-a]azocine-3-carboxylate (3.5 g, 9.93 mmol, 1 eq) in tetrahydrofuran (24 mL) and water (8 mL) was added lithium hydroxide monohydrate (1.04 g, 24.8 mmol, 2.5 eq). The reaction mixture was stirred for 16 h at room temperature. The reaction mixture was partially concentrated under reduced pressure (to remove THF). The crude mixture was diluted with water (40 mL) and EtOAc (60 mL). The phases were separated, and the aqueous phase was acidified with 1 N HCl (pH=1-2) and then the product was extracted with DCM (3×60 mL). The combined organic layers were dried with sodium sulfate, filtered and concentrated under reduced pressure. The crude residue was purified by reverse phase chromatography on 150 g C₁₈ cartridge eluting with 5-60% MeCN in water (with 0.1% formic acid) to give (3S,6S,10aR)-6-{[(tert-butoxy)carbonyl]amino}-9-methyl-5-oxo-1H,2H,3H,5H,6H,7H,10H,10aH-pyrrolo[1,2-a]azocine-3-carboxylic acid (2.85 g, 84.8%) as a white solid. LCMS (ESI): m/z=339.1 (M+H)⁺; ¹H NMR (400 MHz, DMSO-d₆) δ 12.32 (br. s., 1H), 6.75 (d, J=7.3 Hz, 1H), 5.48-5.39 (m, 1H), 4.47 (q, J=7.4 Hz, 1H), 4.19 (d, J=7.1 Hz, 2H), 2.77-2.52 (m, 2H), 2.26-2.03 (m, 4H), 1.83 (br. s., 2H), 1.74 (s, 3H), 1.36 (s, 9H).

Step 2: (1R,7S,10S)-4,4-Dibromo-7-{[(tert-butoxy)carbonyl]amino}-3-methyl-8-oxo-9-azatricyclo[7.3.0.0³,5]dodecane-10-carboxylic acid

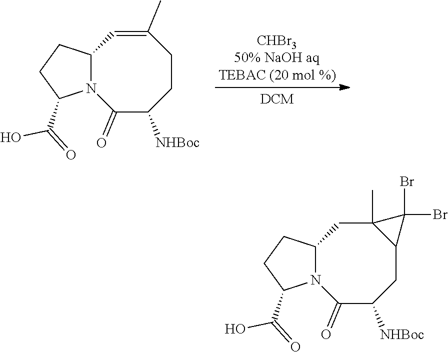

To a solution of (3S,6S,10aR)-6-{[(tert-butoxy)carbonyl]amino}-9-methyl-5-oxo-1H,2H,3H,5H,6H,7H,10H,10aH-pyrrolo[1,2-a]azocine-3-carboxylic acid (1.5 g, 4.43 mmol, 1 eq) in methylene chloride (50 mL) was added tribromomethane (3.84 mL, 44.3 mmol, 10 eq) and benzyltriethylammonium chloride (201 mg, 886 μmol, 0.2 eq) under nitrogen atmosphere. A solution of sodium hydroxide (3.89 g, 97.4 mmol, 22 eq) in water (7.8 mL) was added. The reaction mixture was heated to 40° C. for 24 h. The reaction mixture was concentrated under reduced pressure to remove DCM, water, and most of bromoform. The dark brown residue was dissolved in water (30 mL) and slowly acidified with 3 N aqueous HCl under stirring to pH=2. The acidic aqueous layer was washed with DCM (3×30 mL). The combined organic layers were dried with sodium sulfate, filtered and concentrated under reduced pressure. The crude residue was purified by reverse phase chromatography on 150 g C₁₈ cartridge eluting with 5-60% MeCN (with 0.1% formic acid in water) to give (1R,7S,10S)-4,4-dibromo-7-{[(tert-butoxy)carbonyl]amino}-3-methyl-8-oxo-9-azatricyclo[7.3.0.0^{3,5}]dodecane-10-carboxylic acid (1.42 g, 62.8%) as a beige solid. ¹H NMR (400 MHz, DMSO-d₆) δ 12.45 (br. s., 1H), 6.70 (d, J=6.8 Hz, 1H), 4.55-4.03 (m, 3H), 2.43-2.08 (m, 4H), 2.06-1.97 (m, 1H), 1.96-1.67 (m, 4H), 1.55-1.50 (m, 1H), 1.48-1.44 (m, 1H), 1.42-1.24 (m, 10H).

Step 3: (1R,7S,10S)-4,4-Dibromo-7-{[(tert-butoxy)carbonyl]amino}-3-methyl-8-oxo-9-azatricyclo[7.3.0.0³,5]dodecane-10-carboxylic acid

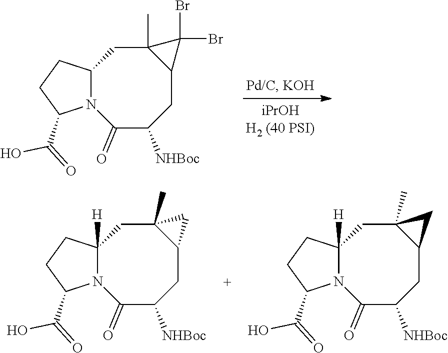

In a pressure vessel, (1R,7S,10S)-4,4-dibromo-7-{[(tert-butoxy)carbonyl]amino}-3-methyl-8-oxo-9-azatricyclo[7.3.0.0^{3,5}]dodecane-10-carboxylic acid (1.4 g, 2.74 mmol, 1 eq) was dissolved in 2-propanol (40 mL) and then, under nitrogen atmosphere, potassium hydroxide (920 mg, 16.4 mmol, 6 eq) and 10% palladium on carbon (50% wet) (1.44 g, 0.68 mmol, 0.25 eq) were added. The reaction mixture was heated to 70° C. under 40 psi of hydrogen for 21 h. The reaction mixture was cooled down to room temperature, diluted with MeOH and filtered over a Celite pad. The filtrate was concentrated under reduced pressure. The crude carboxylate was diluted in minimal amount of water. The crude product was purified by reverse phase chromatography on 50 g C₁₈ cartridge eluting with 5-60% MeCN in water (with 0.1% formic acid in water). The tubes containing the pure product were concentrated under reduced pressure to give the desired product (460 mg) as a white solid (mixture of two isomers). The product was submitted to chiral SFC separation (SFC conditions: Column Lux amylose-1 21.2×250 mm 5 um column, 10 mg/inj, concentration 1.67 mg/mL, Column T=40° C., Flow rate 25 mL/min, 10% MeOH, cycle time: 4.6 min) to give (1R,3S,5R,7S,10S)-7-{[(tert-butoxy)carbonyl]amino}-3-methyl-8-oxo-9-azatricyclo[7.3.0.0^{3,5}]dodecane-10-carboxylic acid (180 mg, 18.6%, fastest eluting isomer) as a white solid. LCMS (ESI): m/z=353.2 (M+H)⁺. ¹H NMR (400 MHz, benzene-d₆) δ 6.30-6.18 (m, 1H), 4.52-4.32 (m, 2H), 3.60-3.47 (m, 1H), 2.25-2.20 (m, 1H), 1.77-1.72 (m, 1H), 1.66-1.53 (m, 3H), 1.46 (s, 9H), 1.42-1.25 (m, 4H), 1.17-1.06 (m, 1H), 0.81 (s, 3H), 0.33-0.30 (m, 1H), 0.04-−0.10 (m, 1H). (1R,3R,5S,7S,10S)-7-{[(tert-butoxy)carbonyl]amino}-3-methyl-8-oxo-9-azatricyclo[7.3.0.0³,5]dodecane-10-carboxylic acid (256 mg, 26.5%, slowest eluting isomer) was also isolated as a white solid. LCMS (ESI): m/z=353.2 (M+H)⁺; ¹H NMR (400 MHz, benzene-d₆) δ 5.33-5.31 (m, 1H), 4.52-4.46 (m, 1H), 4.11-4.09 (m, 1H), 3.30-3.26 (m, 1H), 2.34-2.31 (m, 1H), 2.02-1.86 (m, 1H), 1.62-1.45 (m, 3H), 1.44 (s, 9H), 1.36-1.09 (m, 2H), 1.12-0.90 (m, 2H), 0.75 (s, 3H), 0.46-0.32 (m, 1H), 0.17-0.14 (m, 1H), −0.42 (t, J=4.4 Hz, 1H).

### Synthesis of Linkers:

Synthesis of ((2-((allyloxy)carbonyl)benzo[b]thiophen-5-yl)methyl)phosphonic acid and ((2-((allyloxy)carbonyl)benzo[b]thiophen-5-yl)fluoromethyl)phosphonic acid

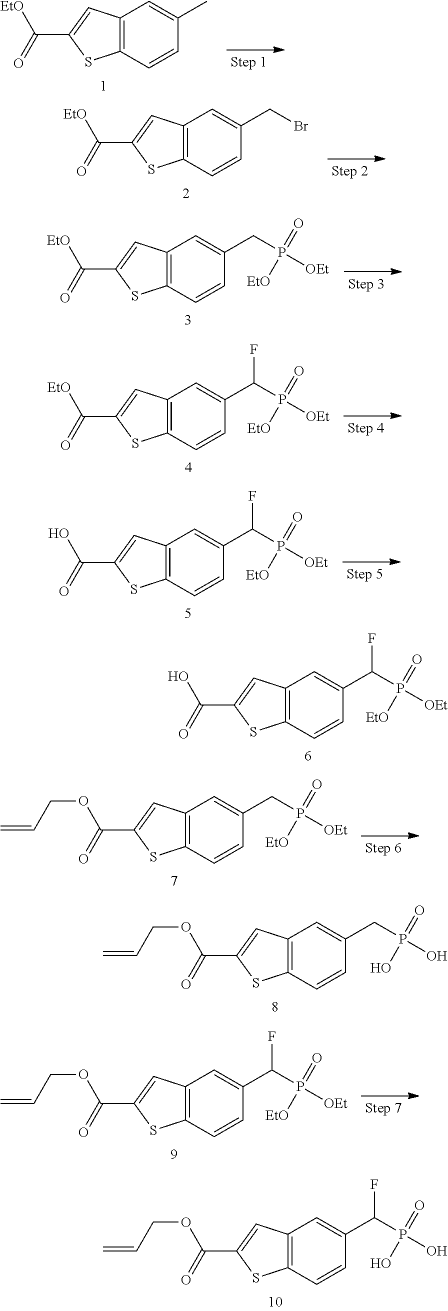

Ethyl 5-methylbenzo[b]thiophene-2-carboxylate was prepared according to the procedure described in WO 2016/100184.

Step 1: ethyl 5-(bromomethyl)benzo[b]thiophene-2-carboxylate

To a solution of ethyl 5-methylbenzo[b]thiophene-2-carboxylate (1, 7.3 kg, 33.1 mol, 1.0 eq.) in CHCl₃ (58 L) stirred at 20° C. was added AIBN (544 g, 3.31 mol, 0.10 eq.) and NBS (6.19 kg, 34.8 mol, 1.05 eq.). The mixture was heated from 30° C. to 50° C. over 4 h and was then heated to 60° C. and stirred for 12 hours. After completion, the reaction mixture was cooled to 10° C. and 15% Na₂SO₃ (20 L) was added. The organic layers were washed with H₂O (20 L*2), dried over Na₂SO₄, and concentrated under reduced pressure at 45° C. to afford ethyl 5-(bromomethyl)benzo[b]thiophene-2-carboxylate (2, 9.50 kg, 23.5 mol, 70.9% yield, 74.0% purity) as a yellow solid. LCMS (ESI): m/z=298.9 [M+H]⁺; ¹H NMR (400 MHz, CDCl₃) δ 8.034 (s, 1H), 7.89-7.84 (m, 2H), 7.50 (d, J=9.6 Hz, 1H), 4.64 (s, 2H), 4.45-4.40 (m, 2H), 1.45-1.41 (m, 3H).

Step 2: ethyl 5-((diethoxyphosphoryl)methyl)benzo[b]thiophene-2-carboxylate

To a solution of ethyl 5-(bromomethyl)benzo[b]thiophene-2-carboxylate (2, 9.50 kg, 31.8 mol, 1.0 eq.) in DMF (28.5 L) stirred at 20° C. was added triethyl phosphite (5.8 kg, 34.9 mol, 1.1 equiv). The mixture was heated to 100° C. and stirred for 5 hrs. After completion, the reaction mixture was cooled to 15° C., poured into H₂O (50.0 L), and extracted with EtOAc (20 L*2). The combined organics were washed with H₂O (20 L*2) and brine (10 L), dried over Na₂SO₄, and concentrated under reduced pressure at 45° C. to give a residue. Crude residue was purified by silica gel chromatography (Petroleum ether/Ethyl acetate=100/1 to 1/1, Petroleum ether/Ethyl acetate=0/1) to afford ethyl 5-((diethoxyphosphoryl)methyl)benzo[b]thiophene-2-carboxylate (3, 5.24 kg, 14 mol, 44.4% yield) as yellow solid. LCMS (ESI): m/z=356.9 [M+H]⁺. ¹H NMR (400 MHz, CDCl₃) δ 8.02 (s, 1H), 7.82-7.80 (m, 2H), 7.41 (d, J=2.0 Hz, 1H), 4.44-4.41 (m, 2H), 4.05-4.01 (m, 4H), 3.27 (d, J=21.6 Hz, 2H), 1.46-1.42 (m, 3H), 1.27-1.23 (m, 6H).

Step 3: ethyl 5-((diethoxyphosphoryl)fluoromethyl)benzo[b]thiophene-2-carboxylate

Three batches were carried out in parallel.

To a solution of ethyl 5-((diethoxyphosphoryl)methyl)benzo[b]thiophene-2-carboxylate (3, 250 g, 702 mmol, 1.00 eq) and N-(benzenesulfonyl)-N-fluorobenzenesulfonamide (221 g, 702 mmol, 1.00 eq) in THF (2.50 L) was added dropwise LiHMDS (1 M, 702 mL, 1.00 eq) at −70° C. under N₂. The mixture was stirred at −70° C. for 3 hrs. Following completion, the reaction mixture was poured into saturated NH₄Cl aqueous solution (5.00 L) slowly at 0° C. and the mixture was stirred at 0° C. for 0.5 hr. Then three batches were combined to workup. The mixture was extracted with ethyl acetate (5.00 L*3). The organic layers were combined, washed with brine (5.00 L), dried over anhydrous Na₂SO₄ and concentrated under reduced pressure. The residue was purified by column chromatography (SiO₂, petroleum ether/ethyl acetate=1/0-3/1, R_{f}=0.30, petroleum ether/ethyl acetate=1/1) to give ethyl 5-((diethoxyphosphoryl)fluoromethyl)benzo[b]thiophene-2-carboxylate (4, 357 g, 928 mmol, 44.1% yield) as yellow oil. LCMS (ESI): m/z=375.0 [M+H]⁺. ¹H NMR (400 MHz, CDCl₃) δ 8.07 (s, 1H), 8.00 (s, 1H), 7.90 (d, J=8.4 Hz, 1H), 7.59 (d, J=8.8 Hz, 1H), 5.88-5.75 (m, 1H), 4.45-4.40 (m, 2H), 4.15-4.05 (m, 4H), 1.45-1.41 (m 3H), 1.31-1.28 (m, 6H).

Step 4: 5-((diethoxyphosphoryl)fluoromethyl)benzo[b]thiophene-2-carboxylic acid

To a solution of ethyl 5-((diethoxyphosphoryl)fluoromethyl)benzo[b]thiophene-2-carboxylate (4, 252 g, 673 mmol, 1.00 eq) in MeOH (1.80 L) was added H₂O (760 mL) and LiOH·H₂O (56.5 g, 1.35 mol, 2.00 eq) at 10-20° C. under N₂. The mixture was stirred at 10-20° C. for 1 hr. TLC (petroleum ether/ethyl acetate=1/1) showed that compound 4 was consumed (R^{f}=0.30) and desired spot (R^{f}=0.10) was formed. The reaction mixture was quenched by H₂O (2.50 L) and then adjusted pH to 3-4 with HCl (aq.1M). The mixture was extracted with dichloromethane (2.50 L*3). The combined organic layers were dried over anhydrous Na₂SO₄ and concentrated under reduced pressure to give 5-((diethoxyphosphoryl)fluoromethyl)benzo[b]thiophene-2-carboxylic acid (5, 222 g, 626 mmol, 93.0% yield) as a white solid.

Step 5: allyl 5-((diethoxyphosphoryl)fluoromethyl)benzo[b]thiophene-2-carboxylate

Two batches were carried out in parallel.

To a solution of 5-((diethoxyphosphoryl)fluoromethyl)benzo[b]thiophene-2-carboxylic acid (5, 178 g, 514 mmol, 1.00 eq) in DMF (1.78 L) was added K₂CO₃ (142 g, 1.03 mol, 2.00 eq) and allyl bromide (68.4 g, 565 mmol, 1.10 eq) at 10-20° C. The mixture was stirred at 10-20° C. for 12 hrs. TLC (petroleum ether/ethyl acetate=0/1) showed that compound 5 was consumed (R^{f}=0.60) and a new spot (R^{f}=0.70) was formed. The reaction mixture was diluted with H₂O (6.00 L), extracted with ethyl acetate (2.00 L*3). The organic layers were combined. The mixture was washed with brine (2.00 L) and NH₄Cl (2.00 L), dried over anhydrous Na₂SO₄ and concentrated under reduced pressure to give allyl 5-((diethoxyphosphoryl)fluoromethyl)benzo[b]thiophene-2-carboxylate (6, 388 g, 985 mmol, 95.8% yield) as yellow oil.

Step 6: ((2-((allyloxy)carbonyl)benzo[b]thiophen-5-yl)methyl)phosphonic acid

To a solution of allyl 5-((diethoxyphosphoryl)methyl)benzo[b]thiophene-2-carboxylate (7, 50 g, 136 mmol, 1.0 eq.) in DCM (5 L) was added TMSBr (411 g, 2.71 mol, 20.0 eq.) dropwise at 0° C. After addition, the reaction mixture was allowed to warm to room temperature and stirred for an additional 12 hrs. The reaction progress was monitored by LCMS. After completion, the reaction mixture was concentrated under reduced pressure and water was added. The resulting mixture was filtered and the filter cake was washed with water (2 L), then dried in vacuum to afford ((2-((allyloxy)carbonyl)benzo[b]thiophen-5-yl)methyl)phosphonic acid (40.3 g, 129 mmol, 95%) as a yellow solid. ¹H NMR (400 MHz, DMSO-d₆) δ 8.20 (s, 1H), 7.96 (d, J=8.4 Hz, 1H), 7.87 (s, 1H), 7.43 (d, J=8.4 Hz, 1H), 6.10-5.98 (m, 1H), 5.46-5.37 (m, 1H), 5.33-5.24 (m, 1H), 4.86-4.77 (m, 2H), 3.08 (d, J=21.2 Hz, 2H). LCMS (ESI) m/z=313.1 [M+H]⁺.

Step 7: ((2-((allyloxy)carbonyl)benzo[b]thiophen-5-yl)fluoromethyl)phosphonic acid

To a solution of allyl 5-((diethoxyphosphoryl)fluoromethyl)benzo[b]thiophene-2-carboxylate (9, 5.2 g, 13.5 mmol, 1.0 eq.) in DCM (500 mL) was added TMSBr (41.1 g, 270 mmol, 20.0 eq.) dropwise at 0° C. After the addition, the reaction mixture was allowed to warm to room temperature and stirred for an additional 12 hrs. The reaction progress was monitored by LCMS. After completion, the reaction mixture was concentrated under reduced pressure and water was added. The resulting mixture was filtered and the filter cake was washed with water (200 mL), then dried in vacuum to afford ((2-((allyloxy)carbonyl)benzo[b]thiophen-5-yl)fluoromethyl)phosphonic acid (3.1 g, 9.37 mmol, 69%) as a yellow solid. ¹H NMR (400 MHz, DMSO-d₆) δ 8.32 (s, 1H), 8.14-8.05 (m, 2H), 7.59 (d, J=8.4 Hz, 1H), 6.12-5.99 (m, 1H), 5.84 (dd, J=44.3, 8.2 Hz, 1H), 5.49-5.40 (m, 1H), 5.35-5.27 (m, 1H), 4.88-4.81 (m, 2H). LCMS (ESI): m/z=329.1 [M−H]⁻.

Chiral separation of allyl 5-((diethoxyphosphoryl)fluoromethyl)benzo[b]thiophene-2-carboxylate

Rac-allyl 5-((diethoxyphosphoryl)fluoromethyl)benzo[b]thiophene-2-carboxylate (617 g, 1.62 mol, 1.00 eq) was purified by SFC to give allyl (S)-5-((diethoxyphosphoryl)fluoromethyl)benzo[b]thiophene-2-carboxylate (Peak 1) and allyl (R)-5-((diethoxyphosphoryl)fluoromethyl)benzo[b]thiophene-2-carboxylate (Peak 2).

Preparative SFC method: Instrument: Waters 350 Preparative SFC. Column: REGIS (S,S) WHELK-O1, 250×50 mm I.D., 10 μm. Mobile phase: A for CO₂ and B for MEOH (Neu). Gradient: B 30%. Flow rate: 220 g/min. Back pressure: 100 bar. Column temperature: 35° C. Wavelength: 220 nm. Cycle-time: 3.3 min.

Analytical SFC method: Column: Kromasil (S,S) WHELK-O1, 50×4.6 mm I.D., 3.5 μm. Mobile phase: A for CO₂ and B for MEOH (0.05% DEA). Gradient: B 5 to 40% Flow rate: 3 mL/min. Back pressure: 100 bar. Column temperature: 35° C. Wavelength: 220 nm.

allyl (S)-5-((diethoxyphosphoryl)fluoromethyl)benzo[b]thiophene-2-carboxylate (Peak 1, 267 g, 685 mmol, 39.7% yield, >99% ee, RT=1.36 min) was obtained as yellow oil. LCMS (ESI): m/z=387.1 [M+H]⁺.

allyl (R)-5-((diethoxyphosphoryl)fluoromethyl)benzo[b]thiophene-2-carboxylate (Peak 2, 270 g, 676 mmol, 39.2% yield, >99% ee, RT=1.55 min) was obtained as yellow oil. LCMS (ESI): m/z=387.1 [M+H]⁺.

Assignment of absolute stereochemical configuration was made by comparison of experimental vibrational circular dichroism (VCD) spectra with theoretical VCD spectra obtained from DFT calculations.

The following intermediates in Table 29 were prepared using the method described above in step 7 for the preparation of ((2-((allyloxy)carbonyl)benzo[b]thiophen-5-yl)fluoromethyl)phosphonic acid and utilizing the appropriate starting materials and modifications.

TABLE 29

| Name | Structure | LCMS |
|---|---|---|
| (S)-((2- ((allyloxy)carbonyl)benzo[b]thiophen-5- yl)fluoromethyl)phosphonic acid | 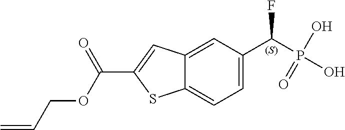 | 329 [M − H]⁻ |
| (R)-((2- ((allyloxy)carbonyl)benzo[b]thiophen-5- yl)fluoromethyl)phosphonic acid | 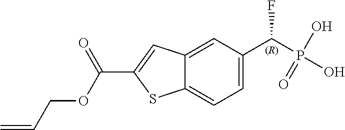 | 329 [M − H]⁻ |

Synthesis of perfluorophenyl 5-((S)-fluoro((R)-(((S)-1-oxo-1-propoxypropan-2-yl)amino)(phenoxy)phosphoryl)methyl)benzo[b]thiophene-2-carboxylate

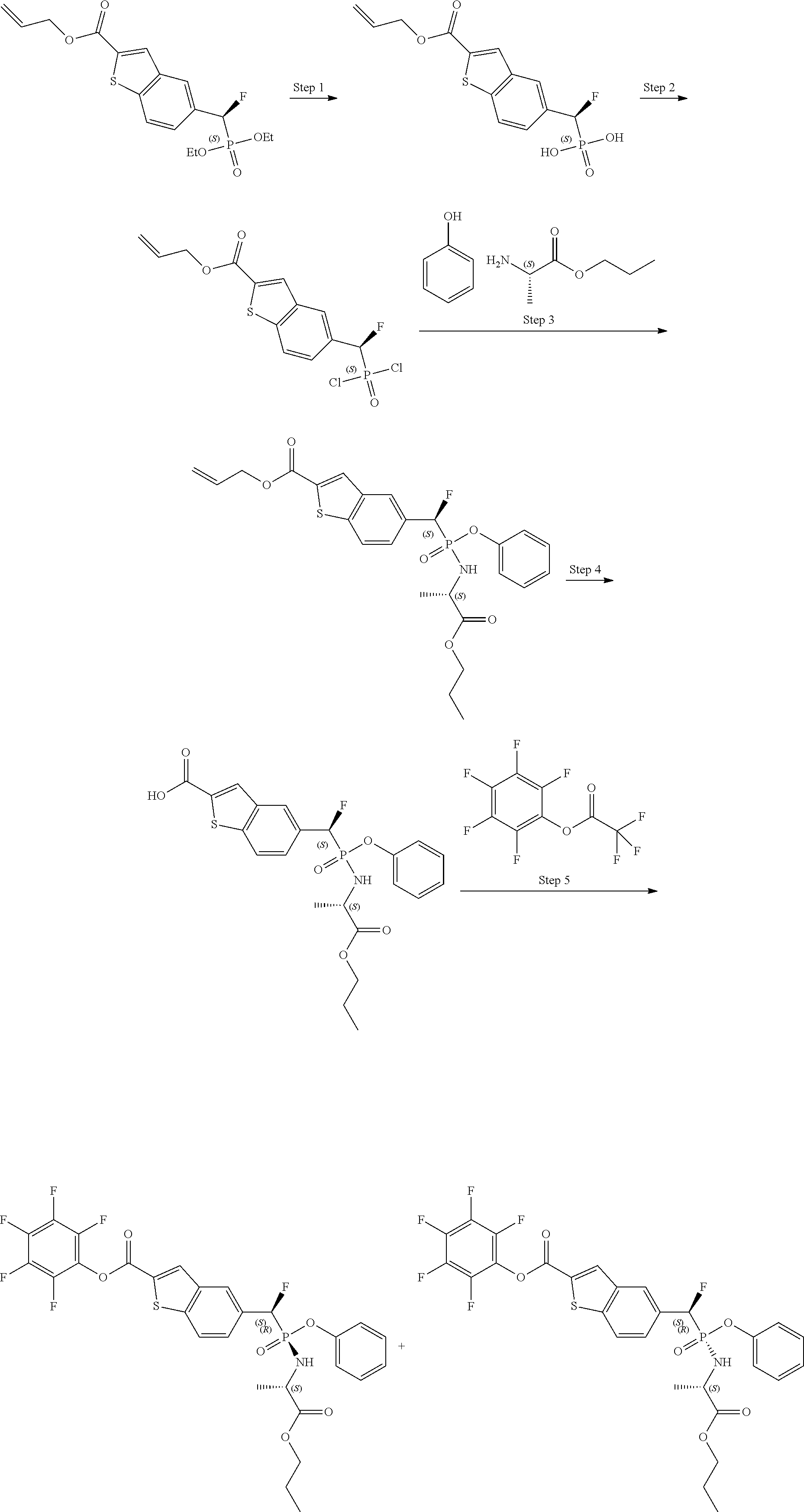

Step 1: Preparation of (S)-((2-((allyloxy)carbonyl)benzo[b]thiophen-5-yl)fluoromethyl)phosphonic acid

To a solution of allyl (S)-5-((diethoxyphosphoryl)fluoromethyl)benzo[b]thiophene-2-carboxylate (20 g, 52 mmol, 1 eq) in methylene chloride (500 mL) was added trimethylsilyl iodide (21 g, 0.10 mol, 2 eq). The mixture was stirred at 0° C. for 1 hour to give a brown solution. The reaction mixture was concentrated under reduced pressure to give a residue. The reaction residue was purified by prep-HPLC (TFA) to lyophilized to give (S)-((2-((allyloxy)carbonyl)benzo[b]thiophen-5-yl)fluoromethyl)phosphonic acid (12 g, 36 mmol, 70% yield) as a brown solid. LCMS (ESI) m/z=330.9

Step 2: Preparation of allyl (S)-5-((dichlorophosphoryl)fluoromethyl)benzo[b]thiophene-2-carboxylate

To a solution of (S)-((2-((allyloxy)carbonyl)benzo[b]thiophen-5-yl)fluoromethyl)phosphonic acid (11 g, 33 mmol, 1 eq) in methylene chloride (200 mL) was added dimethylformamide (0.24 g, 3.3 mmol, 0.1 eq) at 0° C. under N₂ atmosphere, then oxalyl chloride (13 g, 0.10 mol, 3 eq) was added dropwise and stirred at 0° C. for 30 minutes and warmed to 40° C. for 1 hour to give a brown solution. The reaction mixture was concentrated under reduced pressure to give allyl (S)-5-((dichlorophosphoryl)fluoromethyl)benzo[b]thiophene-2-carboxylate (11 g crude) as a yellow solid. LCMS (ESI) m/z=358.9.

Step 3: Preparation of allyl 5-((1S)-fluoro((((S)-1-oxo-1-propoxypropan-2-yl)amino)(phenoxy)phosphoryl)methyl)benzo[b]thiophene-2-carboxylate

To a solution of (S)-5-((dichlorophosphoryl)fluoromethyl)benzo[b]thiophene-2-carboxylate (11 g, 30 mmol, 1 eq) in methylene chloride (150 mL) was added phenol (2.2 g, 24 mmol, 0.8 eq) in methylene chloride (20 mL) over 10 minutes at 0° C., then a solution of N,N-diisopropylethylamine (12 g, 90 mmol, 3 eq) in methylene chloride (200 mL) was added dropwise over 1.5 hours and the mixture was stirred at 25° C. for 5 minutes to give a yellow solution to which a solution of propyl (2S)-2-aminopropanoate (3.9 g, 30 mmol, 1 eq) in methylene chloride (20 mL) was added and the mixture was stirred at 25° C. for 1 hour to give a yellow clean solution. The reaction mixture was concentrated under reduced pressure to give a residue. The residue was purified by flash silica gel chromatography to give allyl 5-((1S)-fluoro((((S)-1-oxo-1-propoxypropan-2-yl)amino)(phenoxy)phosphoryl)methyl)benzo[b]thiophene-2-carboxylate (7.4 g, 14.2 mmol, 47% yield) as a yellow solid. LCMS (ESI) m/z=520.2

Step 4: Preparation of 5-((1S)-fluoro((((S)-1-oxo-1-propoxypropan-2-yl)amino)(phenoxy)phosphoryl)methyl)benzo[b]thiophene-2-carboxylic acid

To a solution of allyl 5-((1S)-fluoro((((S)-1-oxo-1-propoxypropan-2-yl)amino)(phenoxy)phosphoryl)methyl)benzo[b]thiophene-2-carboxylate (3.5 g, 6.7 mmol, 1 eq) in methylene chloride (40 mL) at 0° C. under N₂ atmosphere was added pyrrolidine (0.38 g, 5.4 mmol, 0.8 eq) and palladium;triphenylphosphane (0.7 g, 0.67 mmol, 0.11 eq) dropwise and stirred at 0° C. for 10 minutes and warmed to 25° C. for 10 minutes to give a brown solution. The reaction mixture was concentrated under reduced pressure to give a residue. The reaction residue was purified by prep-HPLC (TFA) to lyophilized to give 5-((1S)-fluoro((((S)-1-oxo-1-propoxypropan-2-yl)amino)(phenoxy)phosphoryl)methyl)benzo[b]thiophene-2-carboxylic acid (2.8 g, 5.9 mmol, 87% yield) as a white solid. ¹H NMR (400 MHz, CDCl₃) δ 8.10 (d, J=11.2 Hz, 2H), 7.97 (d, J=8.4 Hz, 1H), 7.66 (d, J=8.4 Hz, 1H), 7.38-7.30 (m, 2H), 7.19 (d, J=7.6 Hz, 3H), 6.20-5.99 (m, 1H), 4.05-3.77 (m, 3H), 1.68-1.42 (m, 2H), 1.22 (d, J=7.2 Hz, 3H), 0.89-0.85 (m, 3H)

Step 5: Preparation of perfluorophenyl 5-((S)-fluoro((R)-(((S)-1-oxo-1-propoxypropan-2-yl)amino)(phenoxy)phosphoryl)methyl)benzo[b]thiophene-2-carboxylate and perfluorophenyl 5-((S)-fluoro((S)-(((S)-1-oxo-1-propoxypropan-2-yl)amino)(phenoxy)phosphoryl)methyl)benzo[b]thiophene-2-carboxylate

To a solution of 5-((1S)-fluoro((((S)-1-oxo-1-propoxypropan-2-yl)amino)(phenoxy)phosphoryl)methyl)benzo[b]thiophene-2-carboxylic acid (3.0 g, 6.3 mmol, 1 eq) in pyridine (15 mL) at 0° C. under N₂ atmosphere was added 2,3,4,5,6-pentafluorophenyl 2,2,2-trifluoroacetate (5.2 g, 19 mmol, 3 eq) dropwise and stirred at 0° C. for 10 minutes and warmed to 25° C. for 1 hour to give a brown solution. The reaction mixture was concentrated under reduced pressure to give a residue. The residue was purified by flash silica gel chromatography in 3:1 Petroleum ether/ethyl acetate to afford product. The analogous (R)—F isomer was made in a similar manner. Analytical data for all 4 isomers are listed in the table below. SFC conditions used in the peak assignments are as follows: Chiralpak AS-3 50×4.6 mm I.D., 3 um. Mobile phase: Phase A for CO₂, and Phase B for EtOH (0.05% DEA); Gradient elution: B in A from 5% to 40%. Flow rate: 3 mL/min; Detector: PDA; Column Temp: 35 C; Back Pressure: 100 Bar.

| Name | Structure | LCMS | NMR |
|---|---|---|---|
| perfluorophenyl 5- ((S)-fluoro((R)-(((S)- 1-oxo-1- propoxypropan-2- yl)amino)(phenoxy) phosphoryl)methyl) benzo[b]thiophene-2- carboxylate or perfluorophenyl 5- ((S)-fluoro((S)-(((S)- 1-oxo-1- propoxypropan-2- yl)amino)(phenoxy) phosphoryl)methyl) benzo[b]thiophene-2- carboxylate | 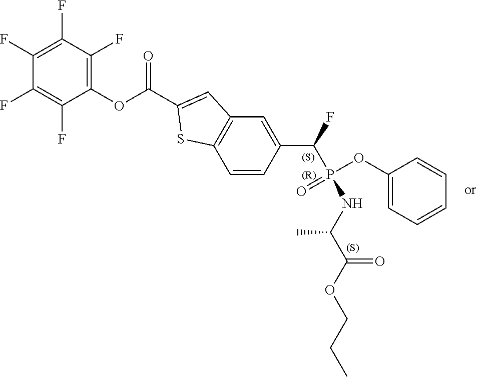 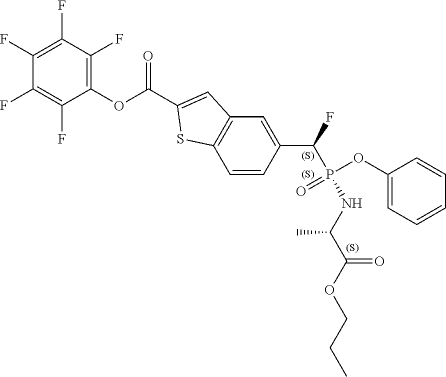 CFTE-27B, SFC Peak 2 Ret. Time = 1.46 Minutes | 645.9 [M + H]⁺ | ¹H NMR (400 MHz, CD₃OD) δ 8.52 (s, 1H), 8.25 (s, 1H), 8.14 (d, J = 8.4 Hz, 1H), 7.81 (d, J = 8.4 Hz, 1H), 7.39-7.32 (m, 2H), 7.27-7.16 (m, 3H), 6.25-6.01 (m, 1H), 4.00 (t, J = 6.8 Hz, 2H), 3.92-3.82 (m, 1H), 1.63-1.52 (m, 2H), 1.14 (d, J = 7.2 Hz, 3H), 0.90 (t, J = 7.2 Hz, 3H) |
| perfluorophenyl 5- ((S)-fluoro((R)-(((S)- 1-oxo-1- propoxypropan-2- yl)amino)(phenoxy) phosphoryl)methyl) benzo[b]thiophene-2- carboxylate or perfluorophenyl 5- ((S)-fluoro((S)-(((S)- 1-oxo-1- propoxypropan-2- yl)amino)(phenoxy) phosphoryl)methyl) benzo[b]thiophene-2- carboxylate | 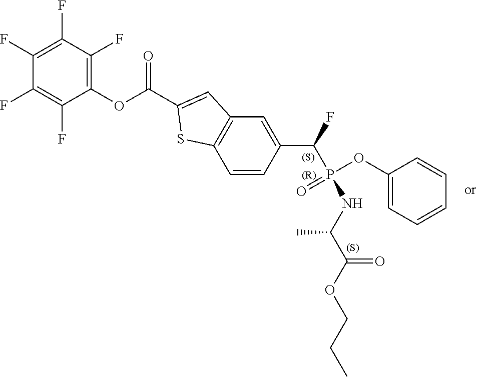 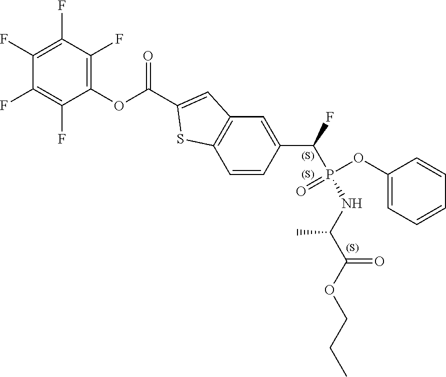 CFTE-27A, SFC Peak 1 Ret. Time = 0.89 Minutes | 646.0 [M + H]⁺ | ¹H NMR (400 MHz, CD₃OD) δ 8.51 (s, 1H), 8.27 (s, 1H), 8.11 (d, J = 8.4 Hz, 1H), 7.79 (d, J = 8.8 Hz, 1H), 7.40-7.33 (m, 2H), 7.25-7.18 (m, 3H), 6.27-6.08 (m, 1H), 4.03-3.85 (m, 3H), 1.63-1.51 (m, 2H), 1.25 (d, J = 7.2 Hz, 3H), 0.89 (t, J = 7.6 Hz, 3H) |
| perfluorophenyl 5- ((R)-fluoro((R)-(((S)- 1-oxo-1- propoxypropan-2- yl)amino)(phenoxy) phosphoryl)methyl) benzo[b]thiophene-2- carboxylate or perfluorophenyl 5- ((R)-fluoro((S)-(((S)- 1-oxo-1- propoxypropan-2- yl)amino)(phenoxy) phosphoryl)methyl) benzo[b]thiophene-2- carboxylate | 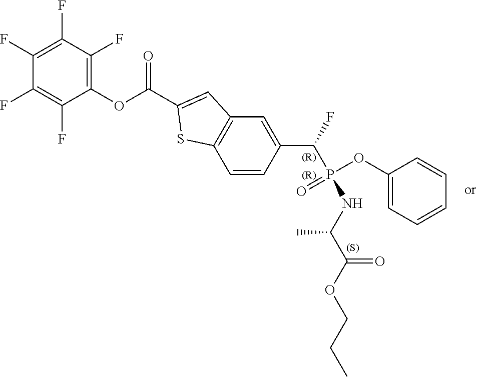 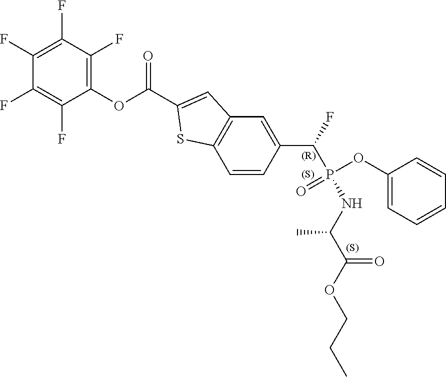 CFTE-28A, SFC Peak 2 Ret. Time = 1.95 Minutes | 645.9 [M + H]⁺ | ¹H NMR (400 MHz, CD₃OD) δ 8.50-8.44 (m, 1H), 8.26-8.18 (m, 1H), 8.13-8.03 (m, 1H), 7.81-7.72 (m, 1H), 7.41-7.28 (m, 2H), 7.27-7.11 (m, 3H), 6.22-6.00 (m, 1H), 4.01-3.94 (m, 2H), 3.93-3.83 (m, 1H), 1.63-1.51 (m, 2H), 1.23 (d, J = 7.2 Hz, 3H), 0.93- 0.83 (m, 3H) |
| perfluorophenyl 5- ((R)-fluoro((R)-(((S)- 1-oxo-1- propoxypropan-2- yl)amino)(phenoxy) phosphoryl)methyl) benzo[b]thiophene-2- carboxylate or perfluorophenyl 5- ((R)-fluoro((S)-(((S)- 1-oxo-1- propoxypropan-2- yl)amino)(phenoxy) phosphoryl)methyl) benzo[b]thiophene-2- carboxylate | 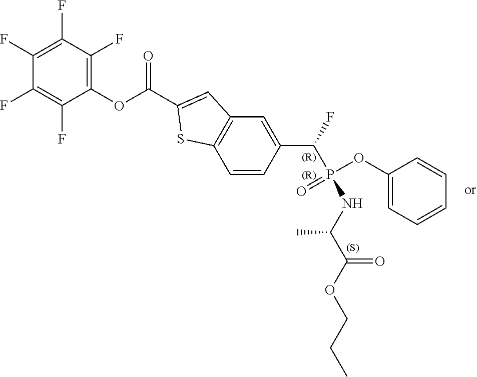 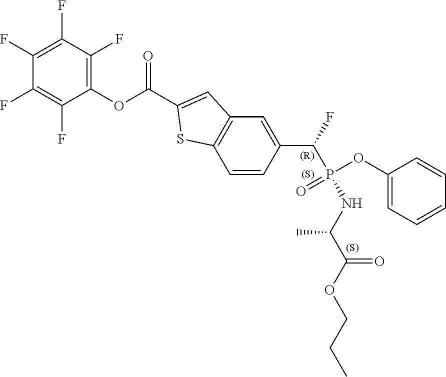 CFTE-28B, SFC Peak 1 Ret. Time = 1.51 Minutes | 646.1 [M + H]⁺ | ¹H NMR (400 MHz, CD₃OD) δ 8.40 (s, 1H), 8.17-8.06 (m, 1H), 8.04-7.95 (m, 1H), 7.75-7.64 (m, 1H), 7.29-7.18 (m, 2H), 7.14-6.98 (m, 3H), 6.18-5.93 (m, 1H), 3.87-3.79 (m, 1H), 3.79-3.67 (m, 2H), 1.48-1.36 (m, 2H), 1.15-1.09 (m, 3H), 0.76 (t, J = 7.6 Hz, 3H) |

The following intermediates in Table 30 were prepared using the synthetic procedure described above for allyl 5-((1S)-fluoro((((S)-1-oxo-1-propoxypropan-2-yl)amino)(phenoxy)phosphoryl)methyl)benzo[b]thiophene-2-carboxylate and utilizing the appropriate starting materials and modifications.

TABLE 30

| Name | Structure | LCMS |
|---|---|---|
| allyl 5-((1R)-fluoro((((S)-1- oxo-1-propoxypropan-2- yl)amino)(phenoxy)phosphoryl) methyl)benzo[b]thiophene-2- carboxylate | 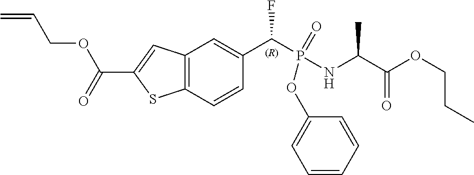 | 520.1 [M + H]⁺ |

The following phosphorus chiral intermediates in Table 31 were obtained by chiral SFC separation of the corresponding P-isomer mixtures, which were prepared using the synthetic procedure described above for allyl 5-((1S)-fluoro((((S)-1-oxo-1-propoxypropan-2-yl)amino)(phenoxy)phosphoryl)methyl)benzo[b]thiophene-2-carboxylate and utilizing the appropriate starting materials and modifications. Absolute stereochemical configuration at phosphorus is arbitrarily assigned as drawn.

TABLE 31

| | | | Preparative SFC |
|---|---|---|---|
| | | | conditions for |
| Name | Structure | LCMS | separation |
| allyl 5-((S)-fluoro((R)- (((S)-1-oxo-1- propoxypropan-2- yl)amino)(phenoxy) phosphoryl)methyl) benzo[b]thiophene-2- carboxylate allyl 5-((S)-fluoro((S)- (((S)-1-oxo-1- propoxypropan-2- yl)amino)(phenoxy) phosphoryl)methyl) benzo[b]thiophene-2- carboxylate | 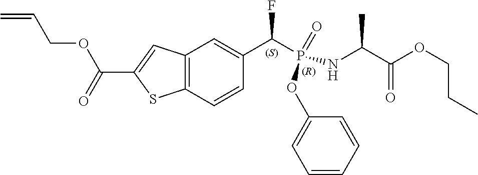 (Peak 1, RT = 4.46 min) 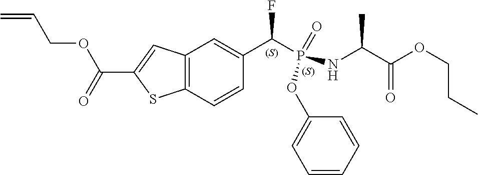 (Peak 2, RT = 6.54 min) | 520.2 [M + H]⁺ 520.2 [M + H]⁺ | ChiralPak R,R- WHELK 250 × 21.2 mm 5 μm column; Mobile phase: A for CO₂ and B for MeOH + 0.1% NH₃H₂O; Gradient: B 50%; Flow rate: 40 mL/min; Back pressure: 100 bar; Column temperature: 35° C.; Wavelength: 220 nm; Cycle-time: 11 min |
| allyl 5-((R)- fluoro((R)-(((S)-1- oxo-1-propoxypropan- 2- yl)amino)(phenoxy) phosphoryl)methyl) benzo[b]thiophene-2- carboxylate allyl 5-((R)-fluoro((S)- (((S)-1-oxo-1- propoxypropan-2- yl)amino)(phenoxy) phosphoryl)methyl) benzo[b]thiophene-2- carboxylate | 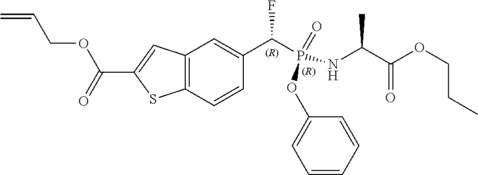 (Peak 1, RT = 2.72 min) 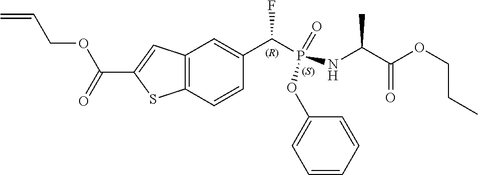 (Peak 2, RT = 4.11 min) | 520.2 [M + H]⁺ 520.2 [M + H]⁺ | ChiralPak IH 250 × 21.2 mm 5 μm column; Mobile phase: A for CO₂ and B for MeOH + 0.1% NH₃H₂O; Gradient: B 35%; Flow rate: 40 mL/min; Back pressure: 100 bar; Column temperature: 35° C.; Wavelength: 220 nm; Cycle-time: 5 min |

The following intermediates in Table 32 were prepared using the synthetic procedure described above for 5-((1S)-fluoro((((S)-1-oxo-1-propoxypropan-2-yl)amino)(phenoxy)phosphoryl)methyl)benzo[b]thiophene-2-carboxylic acid and utilizing the appropriate starting materials and modifications.

TABLE 32

| Name | Structure | LCMS |
|---|---|---|
| 5-((S)-fluoro((R)-(((S)-1-oxo-1- propoxypropan-2- yl)amino)(phenoxy)phosphoryl) methyl)benzo[b]thiophene-2- carboxylic acid | 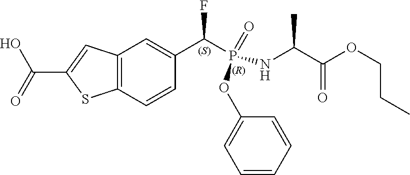 | 480.1 [M + H]⁺ |
| 5-((S)-fluoro((S)-(((S)-1-oxo-1- propoxypropan-2- yl)amino)(phenoxy)phosphoryl) methyl)benzo[b]thiophene-2- carboxylic acid | 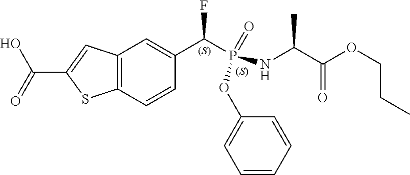 | 480.1 [M + H]⁺ |
| 5-((R)-fluoro((R)-(((S)-1-oxo- 1-propoxypropan-2- yl)amino)(phenoxy)phosphoryl) methyl)benzo[b]thiophene-2- carboxylic acid | 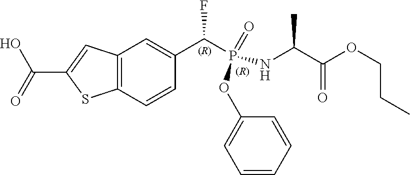 | 480.1 [M + H]⁺ |
| 5-((R)-fluoro((S)-(((S)-1-oxo-1- propoxypropan-2- yl)amino)(phenoxy)phosphoryl) methyl)benzo[b]thiophene-2- carboxylic acid | 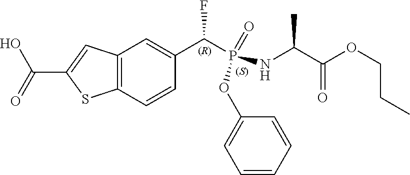 | 480.1 [M + H]⁺ |

The following intermediates in Table 33 were prepared using the protocol described above for synthesis of 5-(((((S)-1-isopropoxy-1-oxopropan-2-yl)amino)(phenoxy)phosphoryl)methyl)benzo[b]thiophene-2-carboxylic acid and utilizing the appropriate starting materials and modifications.

TABLE 33

| Name | Structure | LCMS |
|---|---|---|
| 5-(((((S)-1-ethoxy-1-oxopropan-2- yl)amino)(2,2,2- trifluoroethoxy)phosphoryl)fluoromethyl)benzo [b]thiophene-2-carboxylic acid | 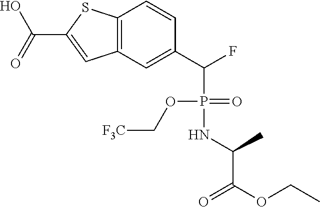 [SMILES: CCOC(=O)[C@H](C)NP(=O)(OCC(F)(F)F)C(F)c1ccc2sc(C(=O)O)cc2c1] | 472.1 [M + H]⁺ |
| 5-(((((S)-1-oxo-1-propoxypropan-2- yl)amino)(2,2,2- trifluoroethoxy)phosphoryl)methyl)benzo[b] thiophene-2-carboxylic acid | 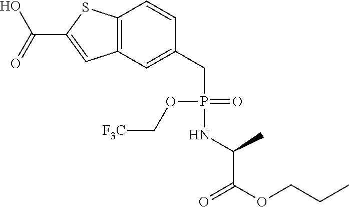 [SMILES: CCCOC(=O)[C@H](C)NP(=O)(Cc1ccc2sc(C(=O)O)cc2c1)OCC(F)(F)F] | 468.0 [M + H]⁺ |
| 5-(fluoro((((S)-1-oxo-1-propoxypropan-2- yl)amino)(2,2,2- trifluoroethoxy)phosphoryl)methyl)benzo[b] thiophene-2-carboxylic acid | 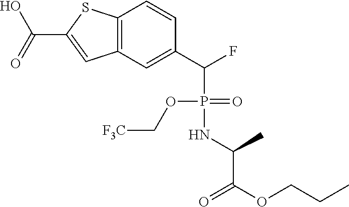 [SMILES: CCCOC(=O)[C@H](C)NP(=O)(OCC(F)(F)F)C(F)c1ccc2sc(C(=O)O)cc2c1] | 485.9 [M + H]⁺ |
| 5-(fluoro((((S)-1-oxo-1-propoxybutan-2- yl)amino)(phenoxy)phosphoryl)methyl)benzo [b]thiophene-2-carboxylic acid | 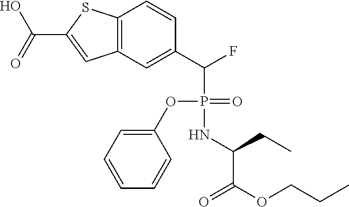 [SMILES: CCCOC(=O)[C@H](CC)NP(=O)(Oc1ccccc1)C(F)c1ccc2sc(C(=O)O)cc2c1] | 494.1 [M + H]⁺ |
| 5-(fluoro((((S)-3-methoxy-1-oxo-1- propoxypropan-2- yl)amino)(phenoxy)phosphoryl)methyl)benzo [b]thiophene-2-carboxylic acid | 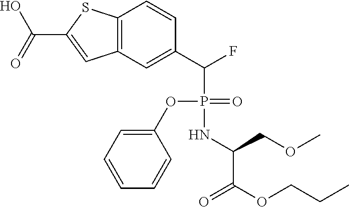 [SMILES: CCCOC(=O)[C@H](COC)NP(=O)(Oc1ccccc1)C(F)c1ccc2sc(C(=O)O)cc2c1] | 510.2 [M + H]⁺ |

Synthesis of (3S,4S)-4-fluoro-1-(pyridin-3-yl)pyrrolidin-3-amine

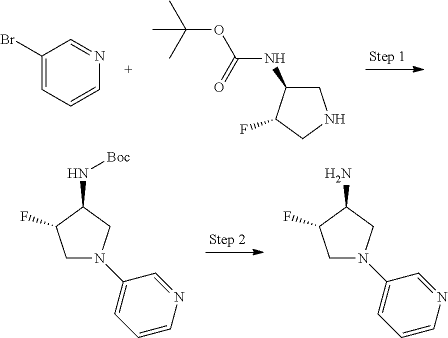 [SMILES: Brc1cccnc1 ; CC(C)(C)OC(=O)N[C@H]1CNC[C@@H]1F ; CC(C)(C)OC(=O)N[C@H]1CN(c2cccnc2)C[C@@H]1F ; N[C@H]1CN(c2cccnc2)C[C@@H]1F]

Step 1: Preparation of tert-butyl ((3S,4S)-4-fluoro-1-(pyridin-3-yl)pyrrolidin-3-yl)carbamate

To a solution of tert-butyl ((3S,4S)-4-fluoropyrrolidin-3-yl)carbamate (0.15 g, 0.73 mmol, 1.0 eq) and 3-bromopyridine (0.15 g, 0.95 mmol, 1.3 eq) in dioxane (1.0 mL) was added Pd₂(dba)₃ (20 mg, 22 μmol, 0.03 eq), Xantphos (38 mg, 66 μmol, 0.09 eq) and Cs₂CO₃ (0.38 g, 1.2 mmol, 1.6 eq). The mixture was stirred at 110° C. for 16 h to give a brown solution. The mixture was diluted with water (10 mL) and extracted with EtOAc (10 mL×3), the combined organic layers were washed with saturated brine (30 mL×2), dried over Na₂SO₄, filtered and concentrated under reduced pressure to give a residue. The residue was purified by flash silica gel chromatography and concentrated in vacuo to give a residue. (0.13 g, 0.48 mmol, 62% yield) as a brown oil. LCMS (ESI) m/z=282.3; ¹H NMR (400 MHz, CDCl₃) δ 8.07-7.97 (m, 2H), 7.20-7.16 (m, 1H), 6.89-6.86 (m, 1H), 5.29-5.09 (m, 1H), 4.77-4.35 (m, 2H), 3.85-3.66 (m, 2H), 3.56-3.42 (m, 1H), 1.46 (s, 9H).

Step 2: Preparation of (3S,4S)-4-fluoro-1-(pyridin-3-yl)pyrrolidin-3-amine

To a solution of tert-butyl N-[(3S,4S)-4-fluoro-1-(pyridin-3-yl)pyrrolidin-3-yl]carbamate (0.13 g, 0.46 mmol, 1.0 eq) in methylene chloride (2.0 mL) was added trifluoroacetic acid (1 mL). The mixture was stirred at 25° C. for 1 h to give a brown solution. The reaction mixture was concentrated under reduced pressure to give (3S,4S)-4-fluoro-1-(pyridin-3-yl)pyrrolidin-3-amine (0.13 g, crude) as a brown oil. LCMS (ESI) m/z=181.9.

Synthesis of 4-(azetidin-3-yl)-1H-pyrazole

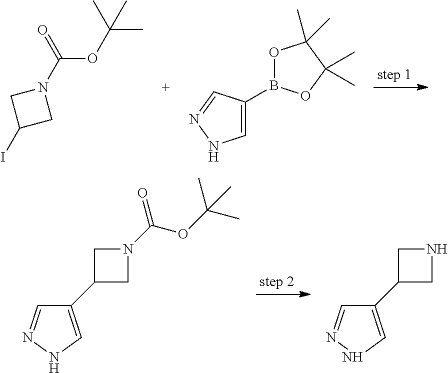

Step 1: Preparation of tert-butyl 3-(1H-pyrazol-4-yl)azetidine-1-carboxylate

To a solution of 4-(4,4,5,5-tetramethyl-1,3,2-dioxaborolan-2-yl)-1H-pyrazole (1.0 g, 5.2 mmol, 1.0 eq) in DMF (20 mL) and water (2.0 mL) was added K₂CO₃ (2.1 g, 15 mmol, 3.0 eq), tert-butyl 3-iodoazetidine-1-carboxylate (2.9 g, 10 mmol, 2.0 eq) and Pd(PPh₃)₄ (0.1 g, 87 μmol, 0.1 eq). The mixture was stirred at 120° C. for 12 h under N₂ to give yellow solution. The mixture was diluted with water (30 mL) and extracted with EtOAc (30 mL×3), the combined organic layers were washed with saturated brine (30 mL×1), dried over Na₂SO₄, filtered and concentrated under reduced pressure to give a residue. The residue was purified by flash silica gel chromatography to give tert-butyl 3-(1H-pyrazol-4-yl)azetidine-1-carboxylate (0.1 g, 0.45 mmol, 8.7% yield) as a yellow oil. LCMS (ESI) m/z=224.1; ¹H NMR (400 MHz, CDCl₃) δ 7.71-7.60 (m, 1H), 7.55 (d, J=2.4 Hz, 1H), 6.34 (t, J=2.0 Hz, 1H), 5.16-5.04 (m, 1H), 4.48-4.36 (m, 2H), 4.35-4.28 (m, 2H), 1.47 (s, 9H)

Step 2: Preparation of 4-(azetidin-3-yl)-1H-pyrazole

To a solution of tert-butyl 3-(1H-pyrazol-4-yl)azetidine-1-carboxylate (100 mg, 0.45 mmol, 1 eq) in DCM (1.5 mL) was added TFA (0.5 mL). The mixture was stirred at 25° C. for 1 h to give a yellow solution. The reaction mixture was concentrated under reduced pressure to give 4-(azetidin-3-yl)-1H-pyrazole (80 mg) as a yellow oil. LCMS (ESI) m/z=124.1

Synthesis of 4-(azetidin-3-yl)-1-methyl-1H-pyrazole

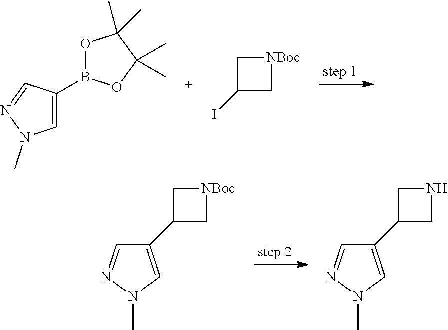

Step 1: Preparation of tert-butyl 3-(1-methyl-1H-pyrazol-4-yl)azetidine-1-carboxylate

To a solution of tert-butyl 3-iodoazetidine-1-carboxylate (2.7 g, 9.6 mmol, 2.0 eq) and 1-methyl-4-(4,4,5,5-tetramethyl-1,3,2-dioxaborolan-2-yl)-1H-pyrazole (1.0 g, 4.8 mmol, 1.0 eq) in dioxane (5.0 ml) was added Pd(PPh₃)₄ (0.55 g, 0.48 mmol, 0.10 eq) and Na₂CO₃ (1.0 g, 9.6 mmol, 2.0 eq). The mixture was stirred at 130° C. for 4 h to give a brown solution. The reaction mixture was concentrated under reduced pressure to give a residue. The residue was diluted with water (50 mL) and extracted with EtOAc (50 mL×3), the combined organic layers were washed with saturated brine (150 mL×2), dried over Na₂SO₄, filtered and concentrated under reduced pressure to give a residue. The residue was purified by RP-flash ((FA condition; 80 g Flash Column Welch Ultimate XB—C18 20-40 μm; H₂O+ACN; 20-35% 20 min) then lyophilized to give tert-butyl 3-(4-methylpyridin-3-yl)azetidine-1-carboxylate (0.36 g, 1.5 mmol, 32% yield) as a brown oil. LCMS (ESI) m/z=237.9; ¹H NMR (400 MHz, CDCl₃) δ 7.58-7.54 (m, 1H), 7.37 (s, 1H), 4.33-4.29 (m, 2H), 3.96 (s, 3H), 3.88-3.84 (m, 2H), 3.72-3.63 (m, 1H), 1.47 (s, 9H).

Step 2: Preparation of 4-(azetidin-3-yl)-1-methyl-1H-pyrazole

To a solution of tert-butyl 3-(1-methyl-1H-pyrazol-4-yl)azetidine-1-carboxylate (80 mg, 0.34 mmol, 1.0 eq) in methylene chloride (1.0 mL) was added trifluoroacetic acid (0.3 mL). The mixture was stirred at 25° C. for 2 h to give a brown solution. The reaction mixture was concentrated under reduced pressure to give 4-(azetidin-3-yl)-1-methyl-1H-pyrazole (0.10 g crude) as a brown oil. LCMS (ESI) m/z=138.0.

Synthesis of 2-(azetidin-3-yl)thiazole

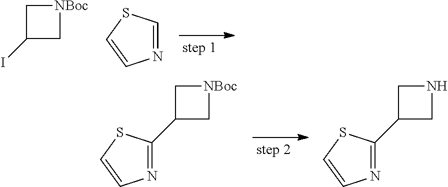

Step 1: Preparation of tert-butyl 3-(thiazol-2-yl)azetidine-1-carboxylate

To a solution of tert-butyl 3-iodoazetidine-1-carboxylate (6.6 g, 23 mmol, 2.0 eq.) and 1,3-thiazole (1.0 g, 12 mmol, 1.0 eq.) in dimethyl sulfoxide (9.0 mL) and water (3.0 mL) was added dipotassium [(sulfonatoperoxy)sulfonyl]oxidanide (6.3 g, 23 mmol,2.0 eq.) and ethylbis(propan-2-yl)amine (6.0 g, 46 mmol, 4.0 eq), then the mixture was stirred at 70° C. for 16 h. The reaction mixture was partitioned between water (100 mL) and EtOAc (100 mL). The organic phase was separated, washed with EtOAc (100 mL×2), dried over Na₂SO₄, filtered and concentrated under reduced pressure to give a residue. The residue was purified by flash silica gel chromatography to get tert-butyl 3-(thiazol-2-yl)azetidine-1-carboxylate was obtained as a white solid. ¹H NMR (400 MHz, CD₃OD) δ 7.77 (d, J=3.20 Hz, 1H), 7.54 (d, J=3.20 Hz, 1H), 4.34-4.40 (m, 2H), 4.17-4.25 (m, 1H), 4.09-4.14 (m, 2H), 1.46 (s, 9H).

Step 2: Preparation of 2-(azetidin-3-yl)thiazole

To a solution of tert-butyl 3-(1,3-thiazol-2-yl)azetidine-1-carboxylate (0.1 g, 0.4 mmol, 1.0 eq.) in methylene chloride (1.0 mL) was added trifluoroacetic acid (0.3 mL) at 25° C. Then the mixture was stirred at 25° C. for 1 h. The mixture was concentrated under reduced pressure to give crude 2-(azetidin-3-yl)-1,3-thiazole (60 mg, crude) as a yellow oil. LCMS (ESI) m/z=140.1.

Synthesis of tert-butyl (R)-3-(4-fluoropyridin-3-yl)pyrrolidine-1-carboxylate

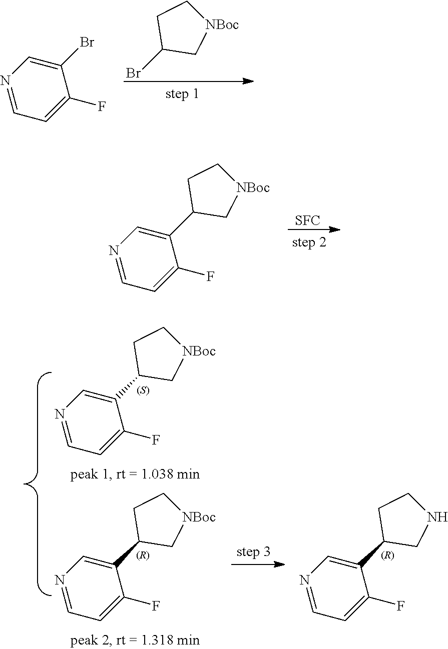

Step 1: Preparation of tert-butyl 3-(4-fluoropyridin-3-yl)pyrrolidine-1-carboxylate

To an 15 mL vial equipped with a stir bar was added 3-bromo-4-fluoropyridine (1.0 g, 5.7 mmol, 1.0 eq), tert-butyl 3-bromopyrrolidine-1-carboxylate (1.84 g, 7.38 mmol, 1.3 eq), Ir[dF(CF₃)ppy]₂(dtbpy)(PF₆) (67 mg, 57 μmol, 0.010 eq), NiCl₂·dtbbpy (35 mg, 85 μmol, 0.015 eq), TTMSS (1.5 g, 5.7 mmol, 1.0 eq), Na₂CO₃ (1.2 g, 11 mmol, 2.0 eq) in DME (20 mL). The vial was sealed and placed under nitrogen was added. The reaction was stirred and irradiated with a 10 W blue LED lamp (3 cm away), with cooling water to keep the reaction temperature at 25° C. for 14 h. The mixture was concentrated directly to give the residue. The residue was purified by column chromatography to give compound tert-butyl 3-(4-fluoropyridin-3-yl)pyrrolidine-1-carboxylate (0.9 g, 3.3 mmol, 60% yield) as a yellow oil. LCMS (ESI) m/z=267.4; ¹H NMR (400 MHz, CD₃OD) δ 8.54-8.42 (m, 2H), 7.29-7.19 (m, 1H), 3.87-3.78 (m, 1H), 3.66-3.56 (m, 2H), 3.44-3.39 (m, 1H), 3.36 (d, J=6.0 Hz, 1H), 2.33 (d, J=2.4 Hz, 1H), 2.23-2.09 (m, 1H), 1.48 (s, 9H).

Step 2: Preparation of (R)-tert-butyl 3-(4-fluoropyridin-3-yl)pyrrolidine-1-carboxylate

The tert-butyl 3-(4-fluoropyridin-3-yl)pyrrolidine-1-carboxylate (0.90 g, 3.4 mmol, 1.0 eq) was purified by SFC (column: DAICEL CHIRALPAK AD (250 mm×30 mm, 10 um), mobile phase: Neu-EtOH; B %: 25%-25%) to give either (S)-tert-butyl 3-(4-fluoropyridin-3-yl)pyrrolidine-1-carboxylate or (R)-tert-butyl 3-(4-fluoropyridin-3-yl)pyrrolidine-1-carboxylate (0.30 g, 1.12 mmol, 33% yield, Rt=1.038 min, peak 1) as a yellow oil and (R)-tert-butyl 3-(4-fluoropyridin-3-yl)pyrrolidine-1-carboxylate or (S)-tert-butyl 3-(4-fluoropyridin-3-yl)pyrrolidine-1-carboxylate (0.28 g, 1.05 mmol, 31% yield, Rt=1.318 min, peak 2) as a yellow oil. ¹H NMR (400 MHz, CD₃OD) δ 8.59-8.41 (m, 2H), 7.24 (d, J=10.2 Hz, 1H), 3.82 (t, J=8.8 Hz, 1H), 3.71-3.54 (m, 2H), 3.51-3.34 (m, 2H), 2.33 (s, 1H), 2.24-2.07 (m, 1H), 1.48 (s, 9H).

Step 3: Preparation of either (R)-4-fluoro-3-(pyrrolidin-3-yl)pyridine or (S)-4-fluoro-3-(pyrrolidin-3-yl)pyridine

To a solution of (R)-tert-butyl 3-(4-fluoropyridin-3-yl)pyrrolidine-1-carboxylate or (S)-4-fluoro-3-(pyrrolidin-3-yl)pyridine (Peak 2, 80 mg, 0.30 mmol, 1.0 eq) in DCM (1.0 mL) was added TFA (0.30 mL), the mixture was stirred at 25° C. for 1 h. The mixture was concentrated directly to give (R)-4-fluoro-3-(pyrrolidin-3-yl)pyridine (50 mg, crude) as a yellow oil. LCMS (ESI) m/z=167.0.

Synthesis of 2-(azetidin-3-yl)oxazole

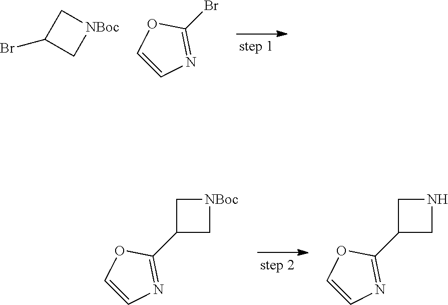 [SMILES: CC(C)(C)OC(=O)N1CC(Br)C1 ; Brc1ncco1 ; CC(C)(C)OC(=O)N1CC(c2ncco2)C1 ; c1coc(C2CNC2)n1]

Step 1: Preparation of tert-butyl 3-(oxazol-2-yl)azetidine-1-carboxylate

To an 15 mL vial equipped with magnetic stir bar was added 2-bromooxazole (0.5 g, 3.4 mmol, 1.0 eq), tert-butyl 3-bromoazetidine-1-carboxylate (1.0 g, 4.4 mmol, 1.3 eq), Ir[dF(CF₃)ppy]₂(dtb (22 mg, 34 mol, 0.01 eq), TTMSS (7.5 mg, 51 mol, 0.015 eq), NiCl₂·dtbbpy (1.8 g, 3.4 mmol, 1.0 eq), Na₂CO₃ (3.7 g, 6.8 mmol, 2.0 eq) was added in DME (0.5 mL). The vial was sealed and placed under nitrogen. The reaction was stirred and irradiated with a 10 W blue LED lamp, with cooling water to keep the reaction temperature at 25° C. for 14 h to give a yellow solution. The reaction mixture was concentrated under reduced pressure to give a residue. The residue was purified by flash silica gel chromatography to give tert-butyl 3-(3-methoxy-1-methyl-1H-pyrazol-4-yl)azetidine-1-carboxylate (90 mg, 41 mmol, 12% yield) as a yellow oil. LCMS (ESI) m/z=225.0.

Step 2: Preparation of 2-(azetidin-3-yl)oxazole

To a solution of tert-butyl 3-(oxazol-2-yl)azetidine-1-carboxylate (0.85 g, 3.8 mmol, 1.0 eq) in DCM (9.0 mL) was added TFA (3.0 mL) to the mixture, the mixture was stirred at 25° C. for 1 h to give a yellow solution. The reaction mixture was concentrated under reduced pressure to give crude product as a yellow oil. LCMS (ESI) m/z=125.2.

Synthesis of 3-(azetidin-3-yl)-4-bromoisoxazole

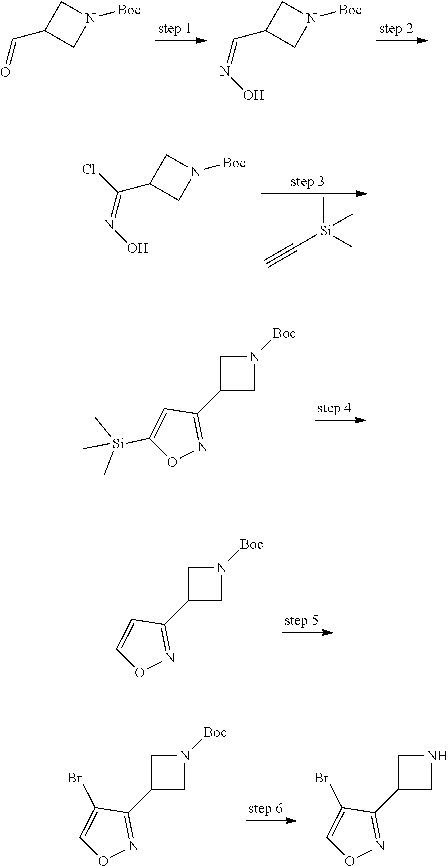

Step 1: Preparation of (Z)-tert-butyl 3-((hydroxyimino)methyl)azetidine-1-carboxylate

To a solution of tert-butyl 3-formylazetidine-1-carboxylate (0.50 g, 2.7 mmol, 1.0 eq) in MeOH (3.0 mL), H₂O (3.0 mL) was added Na₂CO₃ (0.17 g, 1.6 mmol, 0.60 eq) and hydroxylamine hydrochloride (0.22 g, 3.2 mmol, 1.2 eq) at 0° C., the mixture was stirred at 0° C. for 30 minutes and was allowed to warm up to 25° C. for 4 h to give a heterogeneous solution. The reaction mixture was concentrated under reduced pressure to give a residue. The residue was diluted with water (5 mL) and extracted with EtOAc (5 mL×2), the combined organic layers were washed with saturated brine (5 mL×2), dried over Na₂SO₄, filtered and concentrated under reduced pressure to give (Z)-tert-butyl 3-((hydroxyimino)methyl)azetidine-1-carboxylate (0.48 g, crude) as white solid. ¹H NMR (400 MHz, CD₃OD) δ 7.54 (d, J=6.4 Hz, 1H), 4.20-4.14 (m, 1H), 3.95-3.72 (m, 3H), 3.41-3.32 (m, 1H), 1.44 (s, 9H).

Step 2: Preparation of (E)-tert-butyl 3-(chloro(hydroxyimino)methyl)azetidine-1-carboxylate

To a solution of (Z)-tert-butyl 3-((hydroxyimino)methyl)azetidine-1-carboxylate (0.41 g, 2 mmol, 1.0 eq) in DMF (5 mL) was added chlorosuccinimide (0.30 g, 2.2 mmol, 1.1 eq), the mixture was stirred at 40° C. for 4 h to give a yellow solution, the reaction mixture was concentrated under reduced pressure to give a residue. The residue was diluted with water (15 mL) and extracted with EtOAc (20 mL×2), the combined organic layers were washed with saturated brine (10 mL×2), dried over Na₂SO₄, filtered and concentrated under reduced pressure to give (E)-tert-butyl 3-(chloro(hydroxyimino)methyl)azetidine-1-carboxylate (0.44 g, crude) as a white solid.

Step 3: Preparation of tert-butyl 3-(5-(trimethylsilyl)isoxazol-3-yl)azetidine-1-carboxylate

To a solution of (E)-tert-butyl 3-(chloro(hydroxyimino)methyl)azetidine-1-carboxylate (0.44 g, 1.9 mmol, 1 eq) in EtOAc (4 mL) was added trimethylsilylacetylene (0.37 mg, 3.7 mmol, 2.0 eq) and K₂CO₃ (0.37 g, 3.7 mmol, 2.0 eq), the mixture was stirred at 25° C. for 12 h to give a white suspension. The reaction mixture was filtered to give a residue. The residue was purified by prep-HPLC (TFA condition; (column: Welch Ultimate C18 150×25 mm×5 um, mobile phase: water (TFA)-ACN; B %:B %: 40%-60%, 10 min) then lyophilization to give tert-butyl 3-(5-(trimethylsilyl)isoxazol-3-yl)azetidine-1-carboxylate (0.40 g, 1.34 mmol, 72% yield) as brown oil. ¹H NMR (400 MHz, CDCl₃) δ 6.48 (s, 1H), 4.32 (t, J=8.8 Hz, 2H), 4.04 (dd, J=6.0, 8.4 Hz, 2H), 3.96-3.87 (m, 1H), 1.46 (s, 9H), 0.35 (s, 9H).

Step 4: Preparation of tert-butyl 3-(isoxazol-3-yl)azetidine-1-carboxylate

To a solution of tert-butyl 3-(5-(trimethylsilyl)isoxazol-3-yl)azetidine-1-carboxylate (0.20 g, 0.67 mmol, 1.0 eq) in MeOH (2 mL) and a solution of KHF₂ (53 mg, 0.67 mmol, 1.0 eq) in H₂O (1.0 mL) was added to the mixture, the mixture was stirred at 40° C. for 12 h to give a yellow solution, the reaction was purified by prep-HPLC (TFA condition; (column: Welch Ultimate C18 150×25 mm×5 um, mobile phase: water (TFA)-ACN; B %:B %: 20%-40%, 10 min) then lyophilization to give tert-butyl 3-(isoxazol-3-yl)azetidine-1-carboxylate (70 mg, 0.31 mmol 46% yield) as colorless oil. ¹H NMR (400 MHz, CD₃OD) δ 8.64 (d, J=1.6 Hz, 1H), 6.55 (d, J=2.0 Hz, 1H), 4.33 (t, J=8.4 Hz, 2H), 4.07-4.00 (m, 2H), 3.99-3.90 (m, 1H), 1.46 (s, 9H).

Step 5: Preparation of tert-butyl 3-(4-bromoisoxazol-3-yl)azetidine-1-carboxylate

To a solution of tert-butyl 3-(isoxazol-3-yl)azetidine-1-carboxylate (2.5 g, 11 mmol, 1.0 eq) in ACN (20 mL) was added 1-bromopyrrolidine-2,5-dione (3.0 g, 17 mmol, 1.5 eq) and palladium(2+) diacetate (0.25 g, 1.1 mmol, 0.10 eq), the mixture was stirred at 70° C. for 12 h to give a red solution. The mixture was purified by reversed phase (TFA) then lyophilization to give tert-butyl 3-(4-bromoisoxazol-3-yl)azetidine-1-carboxylate (0.20 g, 0.67 mmol, 6.0% yield) as a yellow oil. LCMS (ESI) m/z=303.1 ¹H NMR (400 MHz, CDCl₃) δ 8.42 (s, 1H), 4.37-4.30 (m, 2H), 4.30-4.24 (m, 2H), 3.93-3.80 (m, 1H), 1.46 (s, 9H).

Step 6: Preparation of 3-(azetidin-3-yl)-4-bromoisoxazole

To a solution of tert-butyl 3-(4-bromoisoxazol-3-yl)azetidine-1-carboxylate (50 mg, 0.16 mmol, 1.0 eq) in DCM (0.30 mL) was added TFA (0.10 mL), the mixture was stirred at 25° C. for 0.5 h. The reaction mixture was concentrated under reduced pressure to give 3-(azetidin-3-yl)-4-bromoisoxazole (50 mg, crude) as a yellow oil. LCMS (ESI) m/z=202.9.

Synthesis of 2-(azetidin-3-yl)-5-methyloxazole

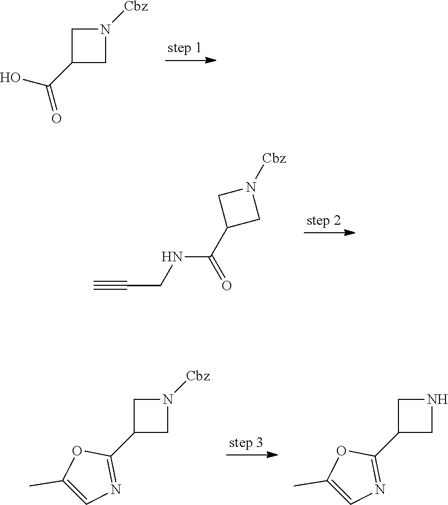

Step 1: Preparation of benzyl 3-(prop-2-yn-1-ylcarbamoyl)azetidine-1-carboxylate

To a solution of 1-((benzyloxy)carbonyl)azetidine-3-carboxylic acid (3.0 g, 13 mmol, 1 eq) in DMF (30 mL) was added prop-2-yn-1-amine (0.84 g, 15 mmol, 1.2 eq) and EDCI (3.7 g, 19 mmol, 1.5 eq), pyridine (2.0 g, 26 mmol, 2 eq). Then the mixture was stirred at 25° C. for 1 h to give a yellow solution. The mixture was diluted with water (50 mL) and extracted with EtOAc (50 mL×2), the combined organic layers were washed with saturated brine (150 mL×2), dried over Na₂SO₄, filtered and concentrated under reduced pressure to give a residue. The residue was purified by flash silica gel chromatography to give benzyl 3-(prop-2-yn-1-ylcarbamoyl)azetidine-1-carboxylate (3.0 g, 11 mmol, 87% yield) as a yellow solid. LCMS (ESI) m/z=272.9; ¹H NMR (400 MHz, CD₃OD) δ 7.26 (m, 5H), 5.00 (s, 2H), 4.03 (m, 5H), 3.89 (d, J=4.0 Hz, 2H), 3.25-3.21 (m, 1H)

Step 2: Preparation of benzyl 3-(5-methyloxazol-2-yl)azetidine-1-carboxylate

To a solution of benzyl 3-(prop-2-yn-1-ylcarbamoyl)azetidine-1-carboxylate (8.0 g, 29 mmol, 1 eq) in toluene (10 mL) was added zinc(2+) ditrifluoromethanesulfonate (0.53 g, 1.5 mmol, 0.050 eq), then the mixture was stirred at 150° C. using microwave for 0.5 h to give a brown solution. The reaction mixture was concentrated under reduced pressure to give a residue. The residue was purified by reverse phase(column:220 g; mobile phase: [water (0.1% TFA)-ACN]; B %: 35%-50%,15 min). The solution was lyophilized to give benzyl 3-(5-methyloxazol-2-yl)azetidine-1-carboxylate (2.8 g, 10 mmol, 35% yield) as a brown oil. LCMS (ESI) m/z=273.0; ¹H NMR (400 MHz, CDCl₃) δ 7.40-7.30 (m, 5H), 6.70 (s, 1H), 5.12 (s, 2H), 4.37-4.32 (m, 2H), 4.29 (m, 2H), 3.93 (s, 1H), 2.31 (s, 3H).

Step 3: Preparation of 2-(azetidin-3-yl)-5-methyloxazole

To a solution of benzyl 3-(5-methyloxazol-2-yl)azetidine-1-carboxylate (0.20 g, 0.73 mmol, 1 eq) in THF (5 mL) was added wet Pd/C (0.2 g, 0.73 mmol, 1 eq) under N₂ atmosphere. Then the mixture was stirred at 25° C. for 4 h under H₂ (15 Psi) atmosphere to give a black suspension. The reaction mixture was filtered and concentrated under reduced pressure to give 2-(azetidin-3-yl)-5-methyloxazole (0.10 g, crude) as a yellow oil. LCMS (ESI) m/z=139.0.

Synthesis of 3-(azetidin-3-yl)-5-(difluoromethyl)isoxazole

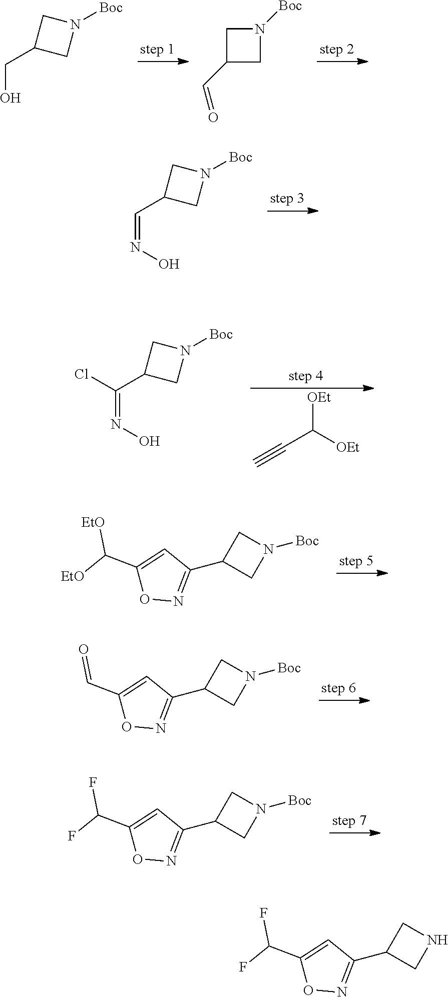

Step 1: Preparation of tert-butyl 3-formylazetidine-1-carboxylate

To a solution of DMSO (1.9 g, 24 mmol, 1.5 eq) in DCM (60 mL) cooled to −70° C. was slowly added oxalyl chloride (3.0 g, 24 mmol, 1.5 eq) under N₂. The reaction mixture stirred at −70° C. for 0.5 h, followed by slow addition of 2-[3-(hydroxymethyl)azetidine-1-carbonyloxy]-2-methylpropan-1-ylium (3 g, 16 mmol, 1.0 eq). The mixture stirred at −70° C. for 1 h and triethylamine (8.1 g, 80 mmol, 5.0 eq) was subsequently added at −70° C. The mixture was warmed and stirred at 25° C. for 0.5 h. The reaction was stirred for an additional 0.5 h at ambient temperatures to give a white turbid mixture. The reaction mixture concentrated under reduced pressure to give tert-butyl 3-formylazetidine-1-carboxylate (2.8 g, crude) as a yellow oil. ¹H NMR (400 MHz, CDCl₃) δ 9.85 (d, J=2.0 Hz, 1H), 5.32-5.25 (m, 1H), 4.17-4.06 (m, 4H), 1.49-1.41 (m, 9H).

Step 2: Preparation of tert-butyl (Z)-3-((hydroxyimino)methyl)azetidine-1-carboxylate

To a solution of tert-butyl 3-formylazetidine-1-carboxylate (2.8 g, 15 mmol, 1.0 eq) in THF/H₂O=1/1 (40 mL) was added hydroxylamine hydrochloride (1.6 g, 30 mmol, 1.5 eq) and K₂CO₃ (2.1 g, 15 mmol, 1.0 eq). The mixture was stirred at 25° C. for 12 h to give a clear yellow solution. The reaction solution was diluted with water (80 mL) and extracted with EtOAc (50 mL×3). The combined organic layers were washed with saturated brine (100 mL×3), dried over Na₂SO₄, filtered, and concentrated under reduced pressure to give tert-butyl (Z)-3-((hydroxyimino)methyl)azetidine-1-carboxylate (2.8 g, crude) as a yellow oil. LCMS (ESI) m/z=201.1.

Step 3: Preparation of tert-butyl (E)-3-(chloro(hydroxyimino)methyl)azetidine-1-carboxylate

To a solution of tert-butyl (Z)-3-((hydroxyimino)methyl)azetidine-1-carboxylate (2.8 g, 14 mmol, 1.0 eq) in DMF (40 mL) was added 1-chloropyrrolidine-2,5-dione (1.9 g, 14 mmol, 1.0 eq). The reaction mixture was stirred at 50° C. for 2 h to give a clear yellow solution. The reaction was diluted with water (100 mL) and extracted with EtOAc (100 mL×3), the combined organic layers were washed with saturated brine (100 mL×3), dried over Na₂SO₄, filtered and concentrated under reduced pressure to give tert-butyl (E)-3-(chloro(hydroxyimino)methyl)azetidine-1-carboxylate (3.0 g, crude) as a yellow solid. LCMS (ESI) m/z=235.1.

Step 4: Preparation of tert-butyl 3-(5-(diethoxymethyl)isoxazol-3-yl)azetidine-1-carboxylate

To a solution of tert-butyl (E)-3-(chloro(hydroxyimino)methyl)azetidine-1-carboxylate (0.2 g, 0.85 mmol, 1.0 eq) in DCM (6.0 mL) was added 3,3-diethoxyprop-1-yne (0.33 g, 2.6 mmol, 3.0 eq) and triethylamine (0.18 g, 1.7 mmol, 2.0 eq). The mixture was stirred at 25° C. for 12 h to give a clear yellow solution. The reaction was filtered and concentrated under reduced pressure to give a residue. The residue was purified by prep-HPLC (TFA condition) and lyophilized to get tert-butyl 3-(5-(diethoxymethyl)isoxazol-3-yl)azetidine-1-carboxylate (0.23 g, 0.70 mmol, 81% yield) as a yellow oil. ¹H NMR (400 MHz, CDCl₃) δ 6.41 (s, 1H), 5.63 (s, 1H), 4.35-4.29 (m, 4H), 4.27-4.15 (m, 1H), 4.10-4.04 (m, 2H), 3.92-3.80 (m, 2H), 1.46 (s, 9H), 1.26 (t, J=7.2 Hz, 6H).

Step 5: Preparation of tert-butyl 3-(5-formylisoxazol-3-yl)azetidine-1-carboxylate

To tert-butyl 3-(5-(diethoxymethyl)isoxazol-3-yl)azetidine-1-carboxylate (0.25 g, 0.76 mol, 1.0 eq) was added a mixture of THF/H₂O/AcOH=1/1/1 (6.0 mL). The reaction was stirred at 25° C. for 12 h to give a yellow solution. The reaction was filtered and concentrated under reduced pressure to give a residue. The residue was purified by prep-HPLC (TFA condition) to get tert-butyl 3-(5-formylisoxazol-3-yl)azetidine-1-carboxylate (80 mg, 0.32 mmol, 42% yield) as a yellow oil. LCMS (ESI) m/z=253.1.

Step 6: Preparation of tert-butyl 3-(5-(difluoromethyl)isoxazol-3-yl)azetidine-1-carboxylate

To a solution of tert-butyl 3-(5-formylisoxazol-3-yl)azetidine-1-carboxylate (0.1 g, 0.40 mmol, 1.0 eq) in DCM (3.0 mL) was cooled to 0° C. and was slowly added diethyl(trifluoro-k⁴-sulfanyl)amine (0.64 g, 4.0 mmol, 10 eq). Then the reaction was warm to 25° C. and stirred at 25° C. for 12 h to give a yellow clear solution. The reaction was filtered and concentrated under reduced pressure to give a residue. The residue was purified by prep-HPLC (FA condition) to lyophilize to give tert-butyl 3-(5-(difluoromethyl)isoxazol-3-yl)azetidine-1-carboxylate (56 mg, 0.20 mmol, 52% yield) as a yellow oil. LCMS (ESI) m/z=275.1.

Step 7: Preparation of 3-(azetidin-3-yl)-5-(difluoromethyl)isoxazole

To a solution of tert-butyl 3-(5-(difluoromethyl)isoxazol-3-yl)azetidine-1-carboxylate (70 mg, 0.26 mmol, 1.0 eq) in TFA/DCM=1/3 (3.0 mL) was stirred at 25° C. for 2 h to give a yellow clean solution. The reaction was concentrated under reduced pressure to give 3-(azetidin-3-yl)-5-(difluoromethyl)isoxazole (45 mg, crude) as a yellow solid. LCMS (ESI) m/z=175.0.

Synthesis of 5-(azetidin-3-yl)-3-methyl-1,2,4-oxadiazole

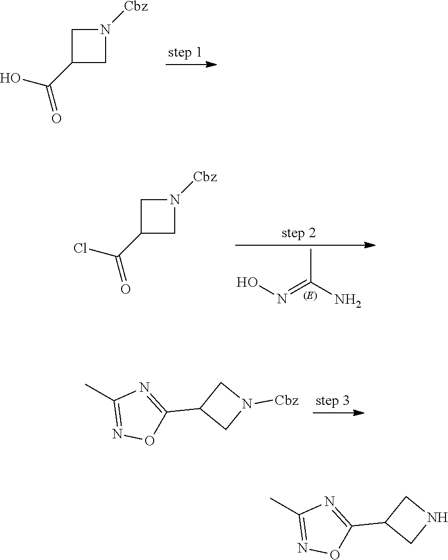

Step 1: Preparation of benzyl 3-(chlorocarbonyl)azetidine-1-carboxylate

To a solution of 1-((benzyloxy)carbonyl)azetidine-3-carboxylic acid (1.0 g, 4.3 mmol, 1.0 eq) in DCM (10 mL) was added DMF (0.10 mL) and oxalyl chloride (2.7 g, 21 mmol, 5.0 eq). The mixture was stirred at 40° C. for 1 h to give a colorless solution. The reaction mixture was concentrated under reduced pressure to give benzyl 3-(chlorocarbonyl)azetidine-1-carboxylate (1.0 g, crude) as a yellow oil.

Step 2: Preparation of benzyl 3-(3-methyl-1,2,4-oxadiazol-5-yl)azetidine-1-carboxylate

To a cooled (0° C.) solution of benzyl 3-(chlorocarbonyl)azetidine-1-carboxylate (1.0 g, 3.94 mmol, 1.0 eq) in DCM (10 mL) was added DIEA (0.56 g, 4.3 mmol, 1.1 eq) and (E)-N′-hydroxyacetimidamide (0.31 g, 4.3 mmol, 1.1 eq) in DCM (5 mL). The mixture was warmed to ambient temperatures and stirred for 2 h to give a colorless solution. The reaction mixture was concentrated and purified by flash silica gel chromatography to give benzyl 3-(3-methyl-1,2,4-oxadiazol-5-yl)azetidine-1-carboxylate (1.0 g, 3.66 mmol, 92% yield) as yellow oil. LCMS (ESI) m/z=274.4.

Step 3: Preparation of 5-(azetidin-3-yl)-3-methyl-1,2,4-oxadiazole

To benzyl 3-(3-methyl-1,2,4-oxadiazol-5-yl)azetidine-1-carboxylate (0.32 g, 1.2 mmol, 1.0 eq) was added a mixture of DCM/TFA=2/1 (2 mL). The mixture was stirred at 70° C. for 2 h. The reaction mixture was concentrated under reduced pressure to give a residue. The reaction was purified by prep-HPLC to lyophilize to give 5-(azetidin-3-yl)-3-methyl-1,2,4-oxadiazole (0.15 g, crude) as a yellow oil. LCMS (ESI) m/z=140.1.

Synthesis of 5-(azetidin-3-yl)-3-methylisoxazole

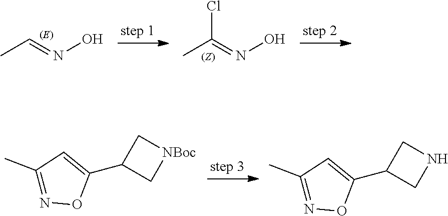

Step 1: Preparation of (Z)—N-hydroxyacetimidoyl chloride

To a solution of (E)-N-ethylidenehydroxylamine (3.0 g, 51 mmol, 1.0 eq) in DMF (50 mL) was added N-chlorosuccinimide (6.8 g, 51 mmol, 1.0 eq) at 25° C. The mixture was stirred at 25° C. for 12 h and subsequently added to water (100 mL). The aqueous mixture was extracted with EtOAc (100 mL×2). The combined organic layers were washed with saturated brine (100 mL×2), dried over Na₂SO₄, filtered, and concentrated under reduced pressure to give (Z)—N-hydroxyacetimidoyl chloride (4.3 g, 46 mmol, 90% yield) as yellow oil. ¹H NMR (400 MHz, CDCl₃) δ 8.89 (s, 1H), 2.25 (s, 3H).

Step 2: Preparation of tert-butyl 3-(3-methylisoxazol-5-yl)azetidine-1-carboxylate

To a solution of (Z)—N-hydroxyacetimidoyl chloride (0.77 g, 8.3 mmol, 1.5 eq) and tert-butyl 3-ethynylazetidine-1-carboxylate (1.0 g, 5.5 mmol, 1.0 eq) in DCM (60 mL) was added triethylamine (1.7 g, 17 mmol, 3.0 eq) at 25° C. The mixture was stirred at for 12 h. The reaction was filtered and concentrated under reduced pressure to give a residue. The residue was purified by flash silica gel chromatography to give tert-butyl 3-(3-methylisoxazol-5-yl)azetidine-1-carboxylate (1.1 g, 4.8 mmol, 87% yield) as yellow oil. LCMS (ESI) m/z=239.1.

Step 3: Preparation of 5-(azetidin-3-yl)-3-methylisoxazole

A solution of tert-butyl 3-(3-methylisoxazol-5-yl)azetidine-1-carboxylate (0.3 g, 1.1 mmol, 1.0 eq) in a mixture of DCM/TFA=3:1 (2.0 mL) was stirred at 25° C. for 1 h. The reaction mixture was concentrated under reduced pressure to give 5-(azetidin-3-yl)-3-methylisoxazole (150 mg, crdue) as a white solid. LCMS (ESI) m/z=139.0.

Synthesis of 5-(azetidin-3-yl)-3-methoxyisoxazole

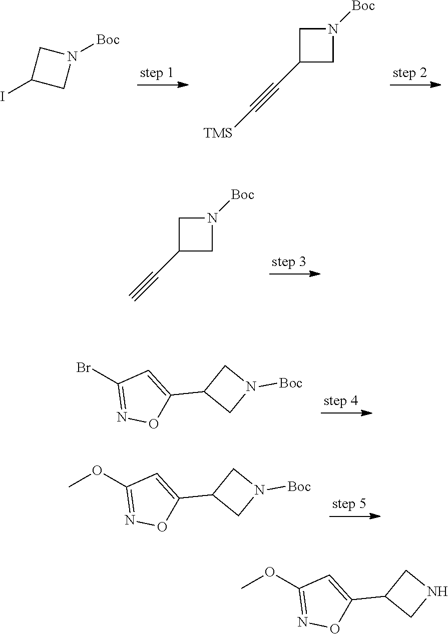 [SMILES: CC(C)(C)OC(=O)N1CC(I)C1 ; CC(C)(C)OC(=O)N1CC(C#C[Si](C)(C)C)C1 ; C#CC1CN(C(=O)OC(C)(C)C)C1 ; CC(C)(C)OC(=O)N1CC(c2cc(Br)no2)C1 ; COc1cc(C2CN(C(=O)OC(C)(C)C)C2)on1 ; COc1cc(C2CNC2)on1]

Step 1: Preparation of tert-butyl 3-((trimethylsilyl)ethynyl)azetidine-1-carboxylate

To a cooled (0° C.) solution of ethynyltrimethylsilane (6.2 g, 63 mmol, 1.2 eq) in THF (200 mL) was slowly added chloro(propan-2-yl)magnesium (5.8 mL, 58 mmol, 1.1 eq, 1.0 M in THF) and the mixture was stirred at 0° C. for 15 minutes. To the mixture was added in a dropwise manner a solution of tert-butyl 3-iodoazetidine-1-carboxylate (15 g, 53 mmol, 1.0 eq) in DMF (300 mL) and after 15 min FeCl₂ (0.54 g, 4.2 mmol, 0.080 eq) was introduced. The mixture was allowed to gradually warm to 25° C. and stirred for 12 h. The reaction was quenched with water (50 mL) and extracted with EtOAc (50 mL×2). The combined organic layers were washed with saturated brine (50 mL×2), dried over Na₂SO₄, filtered and concentrated under reduced pressure to give a residue. The residue was purified by flash silica gel chromatography to give tert-butyl 3-((trimethylsilyl)ethynyl)azetidine-1-carboxylate (7.2 g, 28 mmol, 54% yield) as yellow oil. LCMS (ESI) m/z=254.1.

Step 2: Preparation of tert-butyl 3-ethynylazetidine-1-carboxylate

To a solution of tert-butyl 3-((trimethylsilyl)ethynyl)azetidine-1-carboxylate (1.0 g, 4.1 mmol, 1.0 eq) in MeOH (10 mL) was added K₂CO₃ (0.57 g, 4.1 mmol, 1.0 eq). The mixture was stirred at 25° C. for 2 h and subsequently quenched with water (50 mL). The aqueous layer was extracted with DCM (50 mL×2). The combined organic layers were washed with saturated brine (50 mL×2), dried over Na₂SO₄, filtered, and concentrated under reduced pressure to give tert-butyl 3-ethynylazetidine-1-carboxylate (0.72 g, crude) as yellow oil. LCMS (ESI) m/z=182.0.

Step 3: Preparation of tert-butyl 3-(3-bromoisoxazol-5-yl)azetidine-1-carboxylate

To a solution of tert-butyl 3-ethynylazetidine-1-carboxylate (1.0 g, 5.5 mmol, 1.0 eq) and 1-bromo-N-hydroxymethanecarbonimidoyl bromide (2.2 g, 11 mmol, 2.0 eq) in EtOAc (20 mL) and H₂O (2 mL) was added KHCO₃ (1.7 g, 17 mmol, 3.0 eq). The mixture was stirred at 25° C. for 12 h. The reaction was quenched with water (30 mL) and extracted with DCM (30 mL×2). The combined organic layers were washed with saturated brine (30 mL×2), dried over Na₂SO₄, filtered, and concentrated under reduced pressure to give a residue. The residue was purified by prep-HPLC (FA condition) and lyophilized to give tert-butyl 3-(3-bromoisoxazol-5-yl)azetidine-1-carboxylate (1.4 g, 4.7 mmol, 85% yield) as yellow oil. LCMS (ESI) m/z=304.0; ¹H NMR (400 MHz, CDCl₃) δ 6.27 (s, 1H), 4.30-4.34 (m, 2H), 4.13-4.10 (m, 2H), 4.01-3.99 (m, H), 1.49 (s, 9H).

Step 4: Preparation of tert-butyl 3-(3-methoxyisoxazol-5-yl)azetidine-1-carboxylate

To a solution of tert-butyl 3-(3-bromoisoxazol-5-yl)azetidine-1-carboxylate (1.6 g, 5.2 mmol, 1.0 eq) in methanol (20 mL) was added KOH (3.0 g, 52 mmol, 10 eq). The mixture was stirred at 70° C. for 4 h. The reaction was diluted with water (20 mL) and extracted with EtOAc (20 mL×2). The combined organic layers were washed with saturated brine (20 mL×2), dried over Na₂SO₄, filtered, and concentrated under reduced pressure to give a residue. The residue was purified by flash silica gel chromatography to give tert-butyl 3-(3-methoxyisoxazol-5-yl)azetidine-1-carboxylate (1.0 g, 3.9 mmol, 74% yield) as a yellow oil. LCMS (ESI) m/z=254.2.

Step 5: Preparation of 5-(azetidin-3-yl)-3-methoxyisoxazole

To tert-butyl 3-(3-methoxyisoxazol-5-yl)azetidine-1-carboxylate (1.0 g, 3.9 mmol, 1.0 eq) was diluted a mixture of DCM/TFA=3/1 (2 mL). The mixture was stirred at 25° C. for 1 h. The reaction mixture was concentrated under reduced pressure to give 5-(azetidin-3-yl)-3-methoxyisoxazole (350 mg, crude) as a yellow oil. LCMS (ESI) m/z=154.1.

Synthesis of (3S,4S)-4-fluoro-1-(1H-1,2,4-triazol-3-yl)pyrrolidin-3-amine

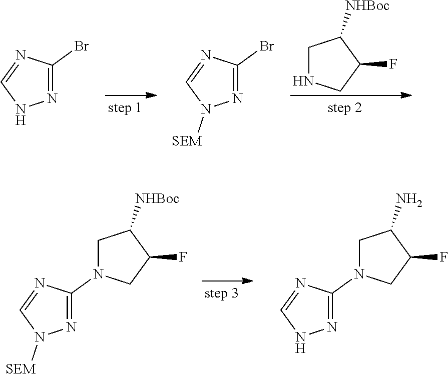

Step 1: Preparation of 3-bromo-1-((2-(trimethylsilyl)ethoxy)methyl)-1H-1,2,4-triazole

To a cooled (0° C.) solution of 3-bromo-1H-1,2,4-triazole (2.0 g, 14 mmol, 1.0 eq.) in THF (20 mL) was added sodium hydride (0.97 g, 42 mmol, 3.0 eq.) in portions. The reaction mixture was allowed to warm to room temperature and stirred for 20 minutes. The mixture was again cooled (0° C.) and (2-(chloromethoxy)ethyl)trimethylsilane (2.7 g, 16 mmol, 2.9 mL, 1.2 eq) was added. The reaction solution was stirred at 25° C. for 2 h and quenched with addition of water (30 mL). The aqueous phase was extracted with EtOAc (30 mL×2). The organic layer was dried with Na₂SO₄, filtered and concentrated to get crude product. The residue was purified by flash silica gel chromatography to get 3-bromo-1-((2-(trimethylsilyl)ethoxy)methyl)-1H-1,2,4-triazole (2.5 g, 9.0 mmol, 67% yield) as a colorless oil. ¹H NMR (400 MHz, CDCl₃) δ 8.12 (s, 1H), 5.49 (d, J=3.2 Hz, 2H), 3.63-3.59 (m, 2H), 0.85-0.82 (m, 2H), 0.00 (s, 9H).

Step 2: Preparation of tert-butyl ((3S,4S)-4-fluoro-1-(1-((2-(trimethylsilyl)ethoxy)methyl)-1H-1,2,4-triazol-3-yl)pyrrolidin-3-yl)carbamate

To a solution of 3-bromo-1-((2-(trimethylsilyl)ethoxy)methyl)-1H-1,2,4-triazole (0.20 g, 0.7 mmol, 1.0 eq) in toluene (2.0 mL) was added tert-butyl ((3S,4S)-4-fluoropyrrolidin-3-yl)carbamate (0.18 g, 0.86 mmol, 1.2 eq), NaOtBu (0.17 g, 1.8 mmol, 2.5 eq.) and palladium catalyst (62 mg, 72 μmol, 0.10 eq, CAS: 1435347-24-2). The mixture was stirred at 80° C. for 6 h. The reaction mixture was filtered and concentrated under reduced pressure to give a residue. The residue was purified by flash silica gel chromatography to give tert-butyl ((3S,4S)-4-fluoro-1-(1-((2-(trimethylsilyl)ethoxy)methyl)-1H-1,2,4-triazol-3-yl)pyrrolidin-3-yl)carbamate (0.23 g, 0.57 mmol, 80% yield) as a colorless oil. ¹H NMR (400 MHz, DMSO-d₆) δ 8.30 (s, 1H), 7.33 (d, J=4.8 Hz, 1H), 5.28 (s, 2H), 5.16-4.94 (m, 1H), 4.18-4.06 (m, 1H), 3.62-3.51 (m, 4H), 3.27 (dd, J=2.8, 10.8 Hz, 1H), 1.39 (s, 9H), 0.87-0.82 (m, 2H), 0.00 (s, 9H).

Step 3: Preparation of (3S,4S)-4-fluoro-1-(1H-1,2,4-triazol-3-yl)pyrrolidin-3-amine

A solution of tert-butyl ((3S,4S)-4-fluoro-1-(1-((2-(trimethylsilyl)ethoxy)methyl)-1H-1,2,4-triazol-3-yl)pyrrolidin-3-yl)carbamate (0.23 g, 0.57 mmol, 1.0 eq) in TFA (0.50 mL) and DCM (0.50 mL) was stirred at 25° C. for 2 h. The reaction mixture was filtered and concentrated under reduced pressure to give (3S,4S)-4-fluoro-1-(1H-1,2,4-triazol-3-yl)pyrrolidin-3-amine (160 mg, crude) as a colorless oil. ¹H NMR (400 MHz, DMSO-d₆) δ 9.00-8.91 (m, 2H), 5.71-5.52 (m, 1H), 4.32-3.66 (m, 5H).

Synthesis of (3S,4S)-4-fluoro-1-(5-methyl-1H-1,2,4-triazol-3-yl)pyrrolidin-3-amine

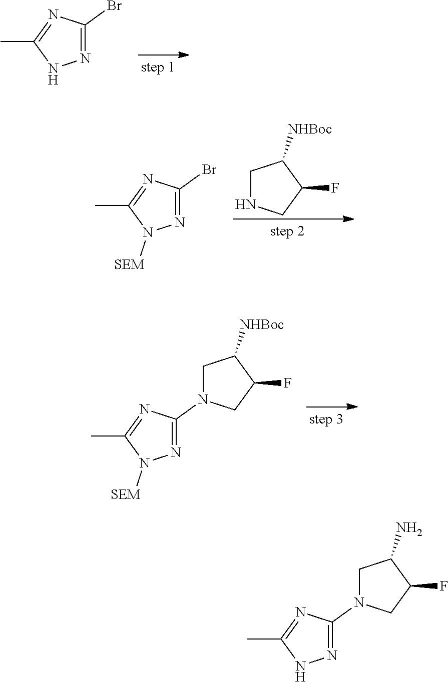

Step 1: Preparation of 3-bromo-5-methyl-1-((2-(trimethylsilyl)ethoxy)methyl)-1H-1,2,4-triazole

To a cooled (0° C.) solution of 3-bromo-5-methyl-1H-1,2,4-triazole (0.5 g, 3.1 mmol, 1.0 eq.) in THF (5.0 mL) was added sodium hydride (0.37 g, 9.2 mmol, 3.0 eq.) in portions. The reaction mixture allowed to warm to room temperature and stirred for 15 minutes. The mixture was again cooled (0° C.) and 2-(chloromethoxy)ethyl-trimethyl-silane (0.67 g, 4.1 mmol, 0.7 mL, 1.2 eq.) was added. The reaction mixture was allowed to warm to ambient temperature and stirred for 2 h, followed by addition of saturated NH₄Cl (10 mL). The aqueous phase was extracted with EtOAc (10 mL×3). The combined organic layers were washed with H₂O (10 mL), dried over Na₂SO₄, filtered, and concentrated under reduced pressure to give a residue. The residue was purified by flash silica gel chromatography to get 3-bromo-5-methyl-1-((2-(trimethylsilyl)ethoxy)methyl)-1H-1,2,4-triazole (0.60 g, 2.1 mmol, 67% yield) as a colorless oil. ¹H NMR (400 MHz, CDCl3) δ 5.44 (s, 2H), 3.59-3.56 (m, 2H), 2.44 (s, 3H), 0.86-0.83 (m, 2H), 0.00 (s, 9H).

Step 2: Preparation of tert-butyl ((3S,4S)-4-fluoro-1-(5-methyl-1-((2-(trimethylsilyl)ethoxy)methyl)-1H-1,2,4-triazol-3-yl)pyrrolidin-3-yl)carbamate

To a solution of 3-bromo-5-methyl-1-((2-(trimethylsilyl)ethoxy)methyl)-1H-1,2,4-triazole (0.20 g, 0.68 mmol, 1.0 eq.) in toluene (2.0 mL) was added tert-butyl ((3S,4S)-4-fluoropyrrolidin-3-yl)carbamate (22 mg, 0.10 mmol, 1.5 eq.), sodium tert-butoxide (0.17 g, 1.8 mmol, 2.5 eq.), and Pd-catalyst (62 mg, 72 μmol, 0.10 eq, CAS: 1435347-24-2). The mixture was stirred at 100° C. for 12 h. The reaction mixture was quenched by sat. NH₄Cl 10 mL at 0° C. and extracted with EtOAc (10 mL×3). The combined organic layers were washed with H₂O (10 mL), dried over Na₂SO₄, filtered and concentrated under reduced pressure to give a residue. The residue was purified by flash silica gel chromatography to give tert-butyl ((3S,4S)-4-fluoro-1-(5-methyl-1-((2-(trimethylsilyl)ethoxy)methyl)-1H-1,2,4-triazol-3-yl)pyrrolidin-3-yl)carbamate (50 mg, 0.12 mmol, 70% yield) as a colorless oil. LCMS (ESI) m/z=416.0.

Step 3: Preparation of (3S,4S)-4-fluoro-1-(5-methyl-1H-1,2,4-triazol-3-yl)pyrrolidin-3-amine

A solution of tert-butyl ((3S,4S)-4-fluoro-1-(5-methyl-1-((2-(trimethylsilyl)ethoxy)methyl)-1H-1,2,4-triazol-3-yl)pyrrolidin-3-yl)carbamate (55 mg, 0.13 mmol, 1.0 eq.) in TFA (0.30 mL) and DCM (0.30 mL) was stirred at 25° C. for 2 h. The reaction mixture was concentrated under reduced pressure to give (3S,4S)-4-fluoro-1-(5-methyl-1H-1,2,4-triazol-3-yl)pyrrolidin-3-amine (50 mg, crude) as a white solid. LCMS (ESI) m/z=185.2.

Synthesis of (3S,4S)-4-fluoro-1-(1-methyl-1H-imidazol-2-yl)pyrrolidin-3-amine

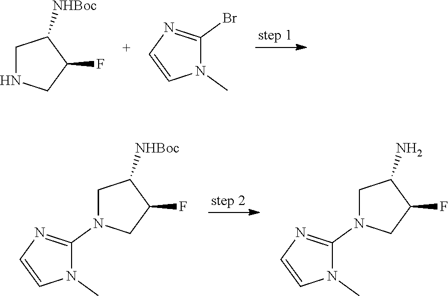

Step 1: Preparation of tert-butyl ((3S,4S)-4-fluoro-1-(1-methyl-1H-imidazol-2-yl)pyrrolidin-3-yl)carbamate

To a solution of 2-bromo-1-methyl-1H-imidazole (66 mg, 0.41 mmol, 1.0 eq) in toluene (1 mL) was added tert-butyl ((3S,4S)-4-fluoropyrrolidin-3-yl)carbamate (99 mg, 0.49 mmol, 1.2 eq), NaOtBu (98 mg, 1.0 mmol, 2.5 eq), and Pd-catalyst (35 mg, 41 μmol, 0.10 eq, CAS: 1435347-24-2). The reaction mixture was stirred at 80° C. for 12 h to give a yellow solution. The reaction mixture was concentrated under reduced pressure to give a residue. The residue was diluted with water (10 mL) and extracted with EtOAc (10 mL×2). The combined organic layers were washed with saturated brine (10 mL×2), dried over Na₂SO₄, filtered, and concentrated under reduced pressure to give a residue. The residue was purified by reversed phase (TFA condition; (column: Phenomenex C18 250×50 mm×10 um, mobile phase: water (TFA)-ACN; B %:B %: 27%-36%,10 min) to give tert-butyl ((3S,4S)-4-fluoro-1-(1-methyl-1H-imidazol-2-yl)pyrrolidin-3-yl)carbamate (0.1 g, 0.35 mmol, 35% yield) as a yellow oil. LCMS (ESI) m/z=285.1; ¹H NMR (400 MHz, CD₃OD) δ 7.19 (dd, J=2.4, 15.2 Hz, 1H), 6.98 (dd, J=2.4, 12.0 Hz, 1H), 4.37-4.16 (m, 1H), 4.13-4.00 (m, 1H), 3.94 (d, J=12.4 Hz, 1H), 3.80 (d, J=5.6 Hz, 1H), 3.65 (d, J=10.4 Hz, 1H), 3.56-3.46 (m, 1H), 3.32 (s, 3H), 1.57-1.41 (m, 9H).

Step 2: Preparation of (3S,4S)-4-fluoro-1-(1-methyl-1H-imidazol-2-yl)pyrrolidin-3-amine

To a solution of tert-butyl ((3S,4S)-4-fluoro-1-(1-methyl-1H-imidazol-2-yl)pyrrolidin-3-yl)carbamate (0.1 g, 0.35 mmol, 1.0 eq) in DCM (1.0 mL) was added trifluoroacetic acid (0.30 mL), the mixture was stirred at 25° C. for 10 minutes to give a yellow clean solution. The reaction mixture was concentrated under reduced pressure to give (3S,4S)-4-fluoro-1-(1-methyl-1H-imidazol-2-yl)pyrrolidin-3-amine (65 mg, crude) as a white oil. LCMS (ESI) m/z=185.1

Synthesis of ((3S,4S)-1-(1,4-dimethyl-1H-imidazol-2-yl)-4-fluoropyrrolidin-3-amine

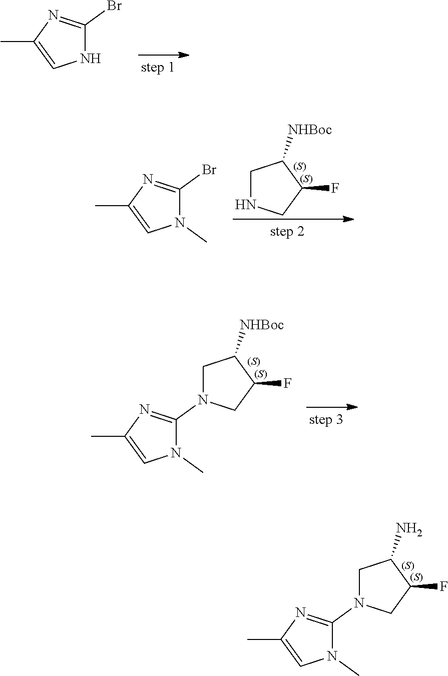

Step 1: Preparation of 2-bromo-1,4-dimethyl-1H-imidazole

To a cooled (0° C.) solution of 2-bromo-4-methyl-1H-imidazole (1.0 g, 6.2 mmol, 1.0 eq.) in THF (10 mL) was added NaH (0.62 g, 16 mmol, 2.5 eq). The mixture stirred for 15 min, followed by dropwise addition of iodomethane (0.88 g, 6.2 mmol, 1.0 eq). The reaction mixture was allowed to warm to ambient temperatures and stirred for 2 h. The mixture subsequently cooled to 0° C. and quenched by addition of sat. NH₄Cl (10 mL). The aqueous phase was extracted with EtOAc (10 mL×3). The combined organic layers were washed with H₂O (10 mL), dried over Na₂SO₄, filtered, and concentrated under reduced pressure to give a residue. The residue was purified by flash silica gel chromatography to give 2-bromo-1,4-dimethyl-1H-imidazole (0.2 g, 1.1 mmol, 19% yield) as a colorless oil. ¹H NMR (400 MHz, CDCl3) δ 7.00 (s, 1H), 3.50 (s, 3H), 2.04 (s, 3H).

Step 2: Preparation of tert-butyl ((3S,4S)-1-(1,4-dimethyl-1H-imidazol-2-yl)-4-fluoropyrrolidin-3-yl)carbamate

A mixture of 2-bromo-1,4-dimethyl-1H-imidazole (0.2 g, 1.1 mmol, 1.0 eq.), tert-butyl ((3S,4S)-4-fluoropyrrolidin-3-yl)carbamate (0.28 g, 1.4 mmol, 1.2 eq.), Pd-catalyst (98 mg, 0.11 mmol, 0.10 eq, CAS: 1435347-24-2), NaO^{t}Bu (0.28 g, 2.89 mmol, 2.5 eq) in toluene (2.0 mL) was stirred at 100° C. for 12 h under N₂. The reaction mixture was cooled to ambient temperatures, followed by addition of sat. NH₄Cl (10 mL) at 20° C. and the mixture was extracted with EtOAc (10 mL×3). The combined organic layers were washed with H₂O (10 mL), dried over Na₂SO₄, filtered, and concentrated under reduced pressure to give a residue. The residue was purified by flash silica gel chromatography to afford tert-butyl ((3S,4S)-1-(1,4-dimethyl-1H-imidazol-2-yl)-4-fluoropyrrolidin-3-yl)carbamate (0.23 g, 0.77 mmol, 68% yield) as a colorless oil. LCMS (ESI) m/z=299.1.

Step 3: Preparation of (3S,4S)-1-(1,4-dimethyl-1H-imidazol-2-yl)-4-fluoropyrrolidin-3-amine

A solution of tert-butyl ((3S,4S)-1-(1,4-dimethyl-1H-imidazol-2-yl)-4-fluoropyrrolidin-3-yl)carbamate (0.23 g, 0.77 mmol, 1.0 eq) in DCM (2.0 mL) and TFA (2.0 mL) was stirred at 25° C. for 2 h. The reaction mixture was concentrated to give (3S,4S)-1-(1,4-dimethyl-1H-imidazol-2-yl)-4-fluoropyrrolidin-3-amine (0.15 g, crude) as a white solid. LCMS (ESI) m/z=199.1.

Synthesis of (3S,4S)-1-(1,5-dimethyl-1H-imidazol-2-yl)-4-fluoropyrrolidin-3-amine

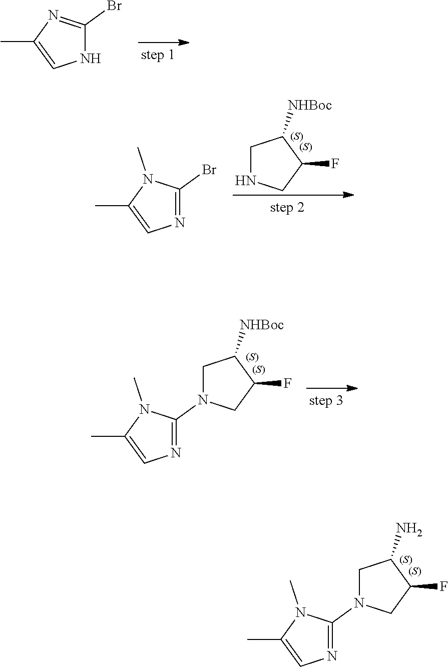

Step 1: Preparation of 2-bromo-1,5-dimethyl-1H-imidazole

To a cooled (0° C.) solution of 2-bromo-4-methyl-1H-imidazole (1.0 g, 6.2 mmol, 1.0 eq.) in THF (10 mL) was added NaH (0.62 g, 16 mmol, 2.5 eq.). The mixture was stirred for 15 min, followed by dropwise addition of iodomethane (0.88 g, 6.2 mmol, 1.0 eq). Then the mixture was stirred at 25° C. for 2 h and subsequently quenched by sat. NH₄Cl (10 mL) at 0° C. The mixture was extracted with EtOAc (10 mL×3). The combined organic layers were washed with H₂O (10 mL), dried over Na₂SO₄, filtered, and concentrated under reduced pressure to give a residue. The residue was purified by flash silica gel chromatography to give 2-bromo-1,5-dimethyl-1H-imidazole (0.2 g, 1.1 mmol, 19% yield) as a colorless oil. LCMS (ESI) m/z=174.8.

Step 2: Preparation of tert-butyl ((3S,4S)-1-(1,5-dimethyl-1H-imidazol-2-yl)-4-fluoropyrrolidin-3-yl)carbamate

To a solution of 2-bromo-1,5-dimethyl-1H-imidazole (0.1 g, 0.57 mmol, 1.0 eq) and tert-butyl ((3S,4S)-4-fluoropyrrolidin-3-yl)carbamate (0.14 g, 0.69 mmol, 1.2 eq) in toluene (2.0 mL) was added NaOtBu (0.14 g, 1.4 mmol, 2.5 eq) and Pd-catalyst (49 mg, 57 μmol, 0.10 eq, CAS: 1435347-24-2) and the resulting mixture was stirred at 85° C. for 12 h. The reaction mixture was concentrated under reduced pressure to give a residue. The residue was purified by prep-TLC to give tert-butyl ((3S,4S)-1-(1,5-dimethyl-1H-imidazol-2-yl)-4-fluoropyrrolidin-3-yl)carbamate (80 mg, 0.27 mmol, 47% yield) as a yellow solid. LCMS (ESI) m/z=299.1.

Step 3: Preparation of (3S,4S)-1-(1,5-dimethyl-1H-imidazol-2-yl)-4-fluoropyrrolidin-3-amine

A solution of tert-butyl ((3S,4S)-1-(1,5-dimethyl-1H-imidazol-2-yl)-4-fluoropyrrolidin-3-yl)carbamate (80 mg, 0.27 mmol, 1.0 eq) in 4M HCl/dioxane (0.50 mL) was stirred at 25° C. for 1 h. The reaction mixture was concentrated under reduced pressure to give (3S,4S)-1-(1,5-dimethyl-1H-imidazol-2-yl)-4-fluoropyrrolidin-3-amine (50 mg, crude) as a yellow oil. LCMS (ESI) m/z=199.1.

Synthesis of 3-(azetidin-3-yl)-4-methyl-4H-1,2,4-triazole

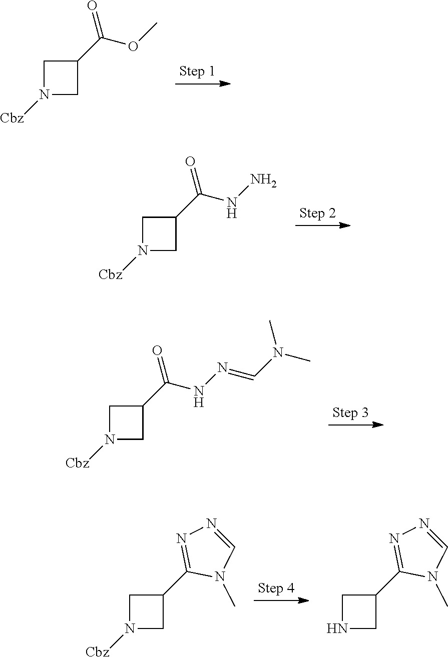

Step 1: Preparation of benzyl 3-(hydrazinecarbonyl)azetidine-1-carboxylate

To a solution of 1-benzyl 3-methyl azetidine-1,3-dicarboxylate (5.0 g, 20 mmol, 1.0 eq) in MeOH (50 mL) was added hydrazine hydrate (6.0 g, 0.12 mol, 6.0 eq.) under N₂ and the mixture was subsequently stirred at 70° C. for 16 h under N₂ atmosphere. The reaction mixture was filtered to give benzyl 3-(hydrazinecarbonyl)azetidine-1-carboxylate (3.2 g, 13 mmol, 64% yield) as a white solid. ¹H NMR (400 MHz, DMSO-d₆) δ 9.16 (s, 1H), 7.30-7.41 (m, 5H), 5.03 (s, 2H), 3.85-4.10 (m, 5H), 3.20-3.27 (m, 1H).

Step 2: Preparation of benzyl (E)-3-(2-((dimethylamino)methylene)hydrazine-1-carbonyl)azetidine-1-carboxylate

To a solution of benzyl 3-(hydrazinecarbonyl)azetidine-1-carboxylate (5.0 g, 20 mmol, 1.0 eq) and (dimethoxymethyl)dimethylamine (5.0 g, 42 mmol, 2.1 eq) in DCM (50 mL) was stirred at 25° C. for 3 h. The mixture was purified by flash silica gel to give benzyl (E)-3-(2-((dimethylamino)methylene)hydrazine-1-carbonyl)azetidine-1-carboxylate (5.0 g, 16 mmol, 82% yield) as a white solid. ¹H NMR (400 MHz, CDCl₃) δ 7.94-7.71 (m, 1H), 7.29-7.26 (m, 3H), 7.25-7.20 (m, 1H), 7.20-7.18 (m, 1H), 5.02 (s, 2H), 4.17 (s, 2H), 4.12-4.00 (m, 2H), 3.80-3.67 (m, 1H), 2.83-2.81 (m, 3H), 2.73 (s, 3H).

Step 3: Preparation of benzyl 3-(4-methyl-4H-1,2,4-triazol-3-yl)azetidine-1-carboxylate

To a solution of benzyl (E)-3-(2-((dimethylamino)methylene)hydrazine-1-carbonyl)azetidine-1-carboxylate (3.0 g, 9.9 mmol, 1.0 eq) in AcOH (60 mL) was added methylamine (3.1 g, 99 mmol, 10 eq) and the mixture was subsequently stirred at 90° C. for 3 h. The reaction mixture was washed with H₂O (60 mL×3), extracted with EtOAc (60 mL×3), dried over Na₂SO₄, filtered and concentrated under reduced pressure to give a residue. The residue was purified by flash silica gel chromatography to give benzyl 3-(4-methyl-4H-1,2,4-triazol-3-yl)azetidine-1-carboxylate (1.7 g, 6.2 mmol, 63% yield) as a yellow oil. ¹H NMR (400 MHz, CDCl₃) δ 8.04 (s, 1H), 7.29-7.18 (m, 5H), 5.07-4.94 (m, 2H), 4.46-4.23 (m, 4H), 3.81 (t, J=7.6 Hz, 1H), 3.49 (s, 3H).

Step 4: Preparation of 3-(azetidin-3-yl)-4-methyl-4H-1,2,4-triazole

To a solution of benzyl 3-(4-methyl-4H-1,2,4-triazol-3-yl)azetidine-1-carboxylate (0.4 g, 1.5 mmol, 1.0 eq.) in THF (4.0 mL) was added wet Pd(OH)₂ (102 mg, 0.73 mmol, 0.50 eq.) and the mixture was stirred at 25° C. for 10 h under H₂ (15 Psi). The mixture was filter over a pad of Celite® and the filtrate was concentrated directly to give 3-(azetidin-3-yl)-4-methyl-4H-1,2,4-triazole (0.2 g, crude). LCMS (ESI) m/z=139.1

Synthesis of 2-(azetidin-3-yloxy)pyrazine

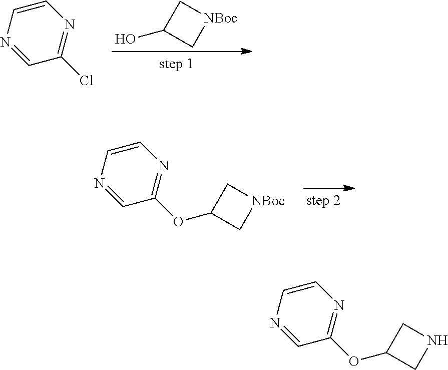 [SMILES: Clc1cnccn1 ; CC(C)(C)OC(=O)N1CC(O)C1 ; CC(C)(C)OC(=O)N1CC(Oc2cnccn2)C1 ; c1cnc(OC2CNC2)cn1]

Step 1: Preparation of tert-butyl 3-(pyrazin-2-yloxy)azetidine-1-carboxylate

To a solution of tert-butyl 3-hydroxyazetidine-1-carboxylate (0.8 g, 4.6 mmol, 1.0 eq) in THF (50 mL) was added NaH (0.45 g, 18 mmol, 4.0 eq.). The solution was stirred at 0° C. for 0.5 h, followed by addition of 2-chloropyrazine (0.53 g, 4.6 mmol, 1.0 eq). The mixture was subsequently warmed and stirred at 60° C. for 12 h. The reaction mixture was quenched with water (50 mL) and extracted with EtOAc (50 mL×3). The combined organic layers were washed with brine (50 mL×3), dried over anhydrous Na₂SO₄, filtered, and concentrated under reduced pressure to give tert-butyl 3-(pyrazin-2-yloxy)azetidine-1-carboxylate (1.14 g, crude) as a yellow oil. ¹H NMR (400 MHz, CDCl₃) δ 8.28 (d, J=1.6 Hz, 1H), 8.18 (d, J=2.8 Hz, 1H), 8.05 (t, J=1.6 Hz, 1H), 5.37-5.25 (m, 1H), 4.41-4.26 (m, 2H), 4.06-3.92 (m, 2H), 1.46 (s, 9H).

Step 2: Preparation of 2-(azetidin-3-yloxy)pyrazine

To a solution of tert-butyl 3-(pyrazin-2.-yloxy)azetidine-1-carboxylate (1.1 g, 4.5 mmol, 1.0 eq) in methylene chloride (12 mL) was added 4 M HCl/dioxane (5 mL, 20.0 mmol, 4.5 eq). The mixture was stirred at 25° C. for 0.5 h and subsequently concentrated under reduced pressure to give 2-(azetidin-3-yloxy)pyrazine (1 g, crude) as a yellow solid.

Synthesis of 4-(azetidin-3-yloxy)-1-methyl-1H-pyrazole

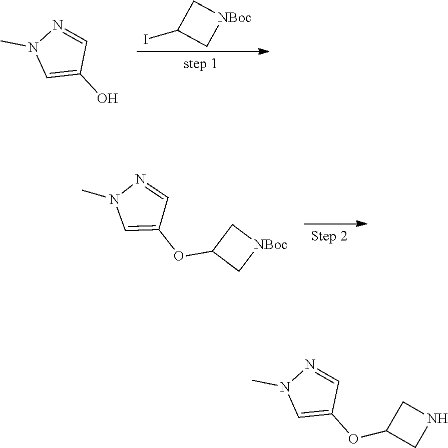

Step 1: Preparation of tert-butyl 3-((1-methyl-1H-pyrazol-4-yl)oxy)azetidine-1-carboxylate

To a solution of 1-methyl-1H-pyrazol-4-ol (0.20 g, 2.0 mmol, 1.0 eq) in DMF (2.0 mL) was added tert-butyl 3-iodoazetidine-1-carboxylate (0.69 g, 2.4 mmol, 1.2 eq) and Cs₂CO₃ (1.3 g, 4 mmol, 2.0 eq) and the reaction mixture was stirred at 60° C. After 12 h, the heterogeneous mixture as cooled to ambient temperatures and quenched with water (10 mL). The mixture was extracted with EtOAc (10 mL×2). The combined organic layers were washed with saturated brine (10 mL×2), dried over Na₂SO₄, filtered, and concentrated under reduced pressure to give a residue. The residue was purified by flash silica gel chromatography to give tert-butyl 3-((1-methyl-1H-pyrazol-4-yl)oxy)azetidine-1-carboxylate (0.20 g, 0.79 mmol, 39% yield) as white solid. LCMS (ESI) m/z=254.1.

Step 2: Preparation of 4-(azetidin-3-yloxy)-1-methyl-1H-pyrazole

To a solution of tert-butyl 3-((1-methyl-1H-pyrazol-4-yl)oxy)azetidine-1-carboxylate (0.10 g, 0.39 mmol, 1.0 eq) in DCM (1.0 mL) was added trifluoroacetic acid (0.3 mL). The mixture was stirred at 25° C. for 0.5 h and subsequently concentrated under reduced pressure to give 4-(azetidin-3-yloxy)-1-methyl-1H-pyrazole (0.10 g, crude) as yellow oil. LCMS (ESI) m/z=154.1.

Synthesis of 2-(azetidin-3-yloxy)pyridine

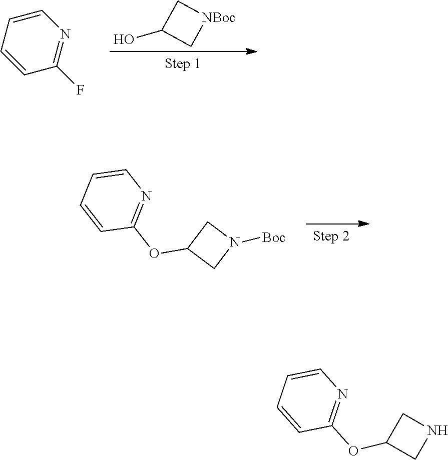 [SMILES: Fc1ccccn1 ; CC(C)(C)OC(=O)N1CC(O)C1 ; CC(C)(C)OC(=O)N1CC(Oc2ccccn2)C1 ; c1ccc(OC2CNC2)nc1]

Step 1: Preparation of tert-butyl 3-(pyridin-2-yloxy)azetidine-1-carboxylate

To a solution of 2-fluoropyridine (0.3 g, 3.1 mmol, 1.0 eq) in THF (3.0 mL) was added tert-butyl 3-hydroxyazetidine-1-carboxylate (0.64 g, 3.7 mmol, 1.2 eq) and KOtBu (0.86 g, 7.7 mmol, 2.5 eq). The reaction mixture was subsequently heated and stirred at 80° C. After 3 h, the reaction was cooled to room temperature and quenched with H₂O (10 mL). The mixture was extracted with EtOAc (10 mL×3). The combined organic layers were washed with sat. brine (10 mL×3), dried over anhydrous Na₂SO₄, filtered, and concentrated under reduced pressure to give a residue. The residue was purified by flash silica gel chromatography to give tert-butyl 3-(pyridin-2-yloxy)azetidine-1-carboxylate (0.19 g, 0.76 mmol, 25% yield) as a yellow oil. ¹H NMR (400 MHz, CDCl₃) δ 8.10 (dd, J=1.2, 5.2 Hz, 1H), 7.62-7.56 (m, 1H), 6.89 (dd, J=5.6, 6.8 Hz, 1H), 6.77 (d, J=8.4 Hz, 1H), 5.35-5.29 (m, 1H), 4.32 (dd, J=6.8, 10.0 Hz, 2H), 3.97 (dd, J=4.0, 10.0 Hz, 2H), 1.45 (s, 9H).

Step 2: Preparation of 2-(azetidin-3-yloxy)pyridine

To a solution of tert-butyl 3-(pyridin-2-yloxy)azetidine-1-carboxylate (0.15 g, 0.6 mmol, 1.0 eq) in DCM (1.5 mL) was added trifluoroacetic acid (0.5 mL). The mixture was stirred at 25° C. for 0.5 h. The reaction mixture was concentrated under reduced pressure to give 2-(azetidin-3-yloxy)pyridine (0.12 g, crude) as a yellow oil. LCMS (ESI) m/z=151.1.

Synthesis of 2-(azetidin-3-yloxy)pyrimidine

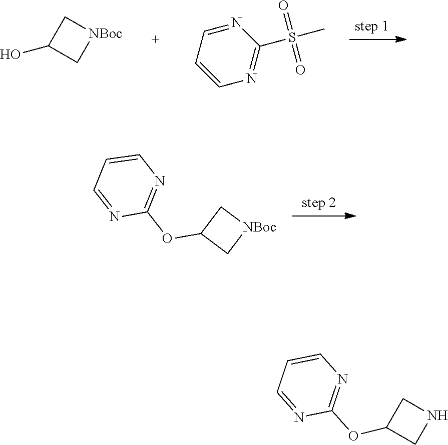

Step 1: Preparation of tert-butyl 3-(pyrimidin-2-yloxy)azetidine-1-carboxylate

To a solution of (2-methanesulfonylpyrimidine (0.2 g, 1.3 mmol, 1.0 eq) in DMF (5.0 mL) was added tert-butyl 3-hydroxyazetidine-1-carboxylate (0.22 g, 1.3 mmol, 1.0 eq) and K₂CO₃ (0.35 g, 2.5 mmol, 2.0 eq). The reaction mixture was stirred at 25° C. for 12 h. The resulting yellow solution was quenched with water (20 mL) and extracted with EtOAc (20 mL×2). The combined organic layers were washed with saturated brine (20 mL×2), dried over Na₂SO₄, filtered, and concentrated under reduced pressure to give a residue. The residue was purified by reversed phase (TFA condition; (column: Phenomenex C18 250×50 mm×10 um, mobile phase: water (TFA)-ACN; B %:B %: 33%-67%,10 min) to lyophilize to give tert-butyl 3-(pyrimidin-2-yloxy)azetidine-1-carboxylate (0.28 g, 1.1 mmol, 88% yield) as a yellow oil. LCMS (ESI) m/z=252.1.

Step 2: Preparation of 2-(azetidin-3-yloxy)pyrimidine

To a solution of tert-butyl 3-(pyrimidin-2-yloxy)azetidine-1-carboxylate (0.1 g, 0.40 mmol, 1.0 eq) in DCM (1.0 mL) was added trifluoroacetic acid (0.30 mL), the mixture was stirred at 25° C. for 10 minutes to give a colorless clean solution. The reaction mixture was concentrated under reduced pressure to give 2-(azetidin-3-yloxy)pyrimidine (45 mg, crude) as a white oil. LCMS (ESI) m/z=152.1

Synthesis of 2-(azetidin-3-yl)-6-methylpyridine

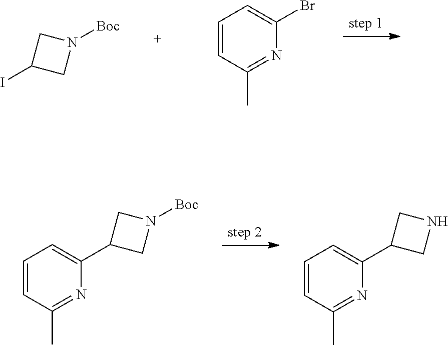

Step 1: Preparation of tert-butyl 3-(6-methylpyridin-2-yl)azetidine-1-carboxylate

A suspension of zinc (2.3 g, 36 mmol, 3 eq) in 1M HCl (1.0 mL) was stirred at 25° C. for 2 h. The suspension was filtered and the filter cake was rinsed with EtOH (10 ml×3) and acetone (10 ml×3), and the solids were subsequently dried under vacuum. The resulting solids were suspended in DMF (5.0 mL) under an atmosphere of N₂ (g) and stirred at 55° C. for 0.5 h. To the mixture was added chlorotrimethylsilane (0.63 g, 5.8 mmol, 0.48 eq), followed by dropwise addition of 1,2-dibromoethane (1.1 g, 5.8 mmol, 0.48 eq). To the resulting mixture was added a solution of tert-butyl 3-iodoazetidine-1-carboxylate (10 g, 36 mmol, 3.0 eq) in DMF (15 mL). After stirring for 0.5 min, the reaction mixture was cooled to room temperature. The resulting organozinc reagent was used without further manipulation.

To a cooled (0° C.) solution of 2-bromo-6-methylpyridine (2.0 g, 12 mmol, 1.0 eq) and Pd(dppf)Cl₂·CH₂Cl₂ (0.85 g, 1.2 mmol, 0.1 eq) in DMF (5.0 mL) was slowly added the organozinc reagent. After complete addition of the zinc reagent, the reaction mixture was warmed to 50° C. for 12 h. The resulting brown suspension was cooled to room temperature and quenched with water (60 mL). The mixture was with EtOAc (60 mL×3). The combined organic layers were washed with saturated brine (80 mL×3), dried over Na₂SO₄, filtered and concentrated in vacuo to give a residue. The residue was purified by column chromatography to give tert-butyl 3-(6-methylpyridin-2-yl)azetidine-1-carboxylate (2.0 g, 8.05 mmol, 69% yield) as a yellow oil. ¹H NMR (400 MHz, CDCl₃) δ 7.57 (t, J=7.6 Hz, 1H), 7.11 (d, J=7.6 Hz, 1H), 7.04 (d, J=7.6 Hz, 1H), 4.36-4.27 (m, 2H), 4.14 (dd, J=6.0, 8.4 Hz, 2H), 3.89 (d, J=5.2 Hz, 1H), 2.56 (s, 3H), 1.47 (s, 9H).

Step 2: Preparation of 2-(azetidin-3-yl)-6-methylpyridine

To a solution of tert-butyl 3-(6-methylpyridin-2-yl)azetidine-1-carboxylate (85 mg, 0.34 mmol) in TFA (0.3 mL) and DCM (1.0 mL) was stirred at 25° C. for 1 h to give a yellow solution. The reaction mixture was subsequently concentrated under reduced pressure to give 2-(azetidin-3-yl)-6-methylpyridine (51 mg, crude) as a yellow oil. LCMS (ESI) m/z=149.1.

Synthesis of tert-Butyl (1S,5S)-2-acetyl-2,6-diazabicyclo[3.2.0]heptane-6-carboxylate and tert-butyl (1R,5R)-2-acetyl-2,6-diazabicyclo[3.2.0]heptane-6-carboxylate

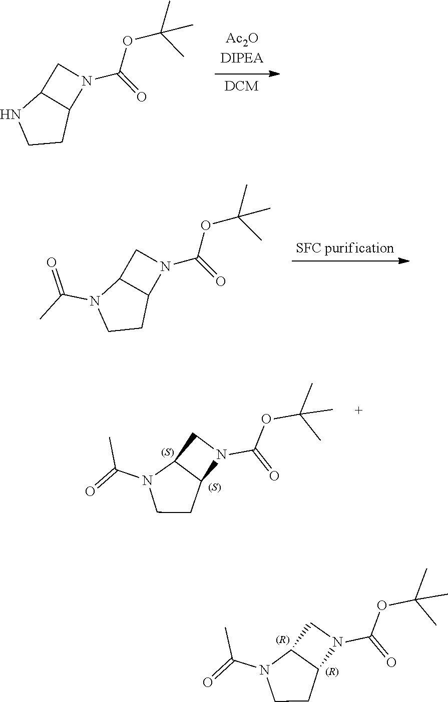

To a solution of tert-butyl 2,6-diazabicyclo[3.2.0]heptane-6-carboxylate (300 mg, 1.51 mmol, 1 eq) and N,N-diisopropylethylamine (1.57 mL, 9.06 mmol, 6 eq) in methylene chloride (5 mL) at 0° C. was slowly added acetic anhydride (170 μL, 1.81 mmol, 1.2 eq). The reaction mixture was stirred 3 h at room temperature and then diluted with DCM and washed with brine, dried with sodium sulfate, filtered and concentrated in vacuo. The crude residue was purified by reverse phase chromatography using a 50 g C₁₈ cartridge eluting with a 5-100% gradient of MeCN in water (with 0.1% formic acid) to give the desired product (313 mg) as an orange oil (mixture of two isomers). The product was submitted to chiral SFC separation (SFC conditions: Column Lux i-Cellulose-3 21.2×250 mm 5 um column, 10.6 mg/inj, concentration 53 mg/mL, Column T=40° C., Flow rate 55 mL/min, 5% MeOH, cycle time: 10.5 min) to give tert-butyl (1S,5S)-2-acetyl-2,6-diazabicyclo[3.2.0]heptane-6-carboxylate (137 mg, 37.8%, fastest eluting isomer) as a white solid. LCMS (ESI): m/z=241.1 (M+H)⁺. Also, tert-butyl (1R,5R)-2-acetyl-2,6-diazabicyclo[3.2.0]heptane-6-carboxylate (132 mg, 36.4%, slowest eluting isomer) was isolated as a white solid. LCMS (ESI): m/z=241.1 (M+H)⁺.

Step 2a: 1-[(1S,5S)-2,6-Diazabicyclo[3.2.0]heptan-2-yl]ethan-1-one TFA salt

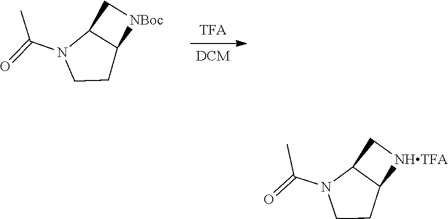

To a solution of tert-butyl (1S,5S)-2-acetyl-2,6-diazabicyclo[3.2.0]heptane-6-carboxylate (137 mg, 570 μmol, 1 eq) in methylene chloride (2 mL) was added trifluoroacetic acid (1 mL). The resulting yellow solution was stirred for 1 h at room temperature. The reaction mixture was concentrated under reduced pressure. The crude TFA salt was used directly for the next step without further purification. LCMS (ESI): m/z=141.1 (M+H)⁺.

Step 2b: 1-[(1R,5R)-2,6-Diazabicyclo[3.2.0]heptan-2-yl]ethan-1-one TFA salt

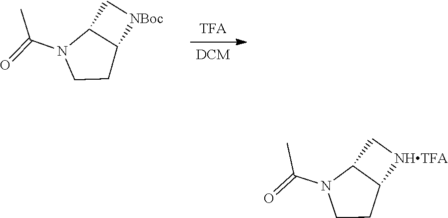

To a solution of tert-butyl (1R,5R)-2-acetyl-2,6-diazabicyclo[3.2.0]heptane-6-carboxylate (132 mg, 549 μmol, 1 eq) in methylene chloride (2 mL) was added trifluoroacetic acid (1 mL). The resulting yellow solution was stirred for 1 h at room temperature. The reaction mixture was concentrated under reduced pressure. The crude TFA salt was used directly for the next step without further purification. LCMS (ESI): m/z=141.1 (M+H)⁺.

Synthesis of 1-(oxetan-3-yl)-1,2,3,4-tetrahydroquinoxaline

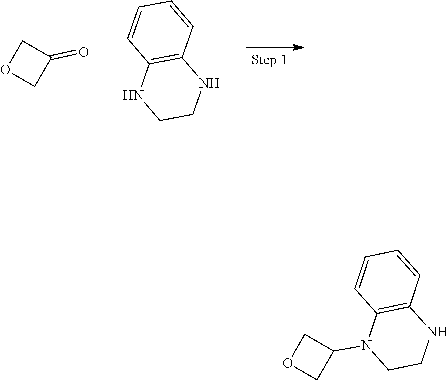

Step 1: Preparation of 1-(oxetan-3-yl)-1,2,3,4-tetrahydroquinoxaline

To a solution of 1,2,3,4-tetrahydroquinoxaline (0.5 g, 3.7 mmol, 1.0 eq) and NaBH(OAc)₃ (0.95 g, 4.5 mmol, 1.2 eq) in methylene chloride (5.0 mL) was added oxetan-3-one (0.32 g, 4.5 mmol, 1.2 eq), and acetic acid (1.1 g, 19 mmol, 5.0 eq). The mixture was stirred at 25° C. for 1 h. The reaction mixture was diluted with DCM (10 mL) and saturated aqueous NAHCO₃ (20 mL) was slowly introduced to the mixture. The mixture was extracted with DCM (3×20 mL). The combined organic layer was dried over Na₂SO₄, filtered, and concentrated under reduced pressure to give 1-(oxetan-3-yl)-1,2,3,4-tetrahydroquinoxaline (0.90 g, crude) as a red oil. LCMS (ESI) m/z=191.1.

Synthesis of tert-butyl 3-(4-(3-fluoro-3-(methoxymethyl)azetidin-1-yl)pyridin-3-yl)azetidine-1-carboxylate

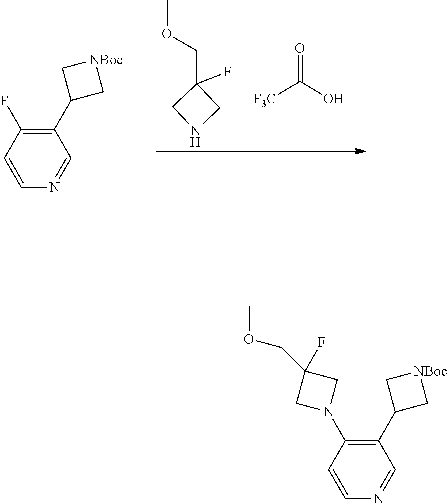

Step 1: Preparation of tert-butyl 3-(4-(3-fluoro-3-(methoxymethyl)azetidin-1-yl)pyridin-3-yl)azetidine-1-carboxylate

To a solution of tert-butyl 3-(4-fluoropyridin-3-yl)azetidine-1-carboxylate (0.48 g, 1.9 mmol, 1.0 eq), 3-fluoro-3-(methoxymethyl)azetidine; trifluoroacetic acid (0.90 g, 3.8 mmol, 2.0 eq) in dimethylacetamide (5.0 mL) was added caesium carbonate (1.8 g, 5.7 mmol, 3.0 eq). The mixture was stirred at 120° C. for 12 hours. The mixture was filtered and the filterate was purified by reversed-phase (FA condition) then lyophilization to get tert-butyl 3-{4-[3-fluoro-3-(methoxymethyl)azetidin-1-yl]pyridin-3-yl}azetidine-1-carboxylate (0.62 g, 1.7 mmol, 92% yield) as a white solid. LCMS (ESI) m/z=352.1. ¹H NMR (400 MHz, CDCl₃) δ=8.41-8.26 (m, 2H), 6.37 (d, J=6.0 Hz, 1H), 4.30-4.02 (m, 8H), 3.84-3.77 (m, 1H), 3.73 (d, J=18.4 Hz, 2H), 3.48 (s, 3H), 1.47 (s, 9H)

Below compounds made with same general procedure:

| Name | Structure | LC-MS | H-NMR data |
|---|---|---|---|
| tert-butyl (S)-3-(4-(2- (methoxymethyl)azetidin- 1-yl)pyridin-3- yl)azetidine-1- carboxylate | 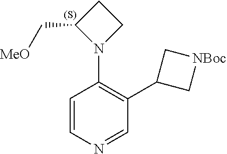 | 334.1 [M + H]⁺ | ¹H NMR (400 MHz, CDCl₃) δ = 8.47 (s, 1H), 8.20 (d, J = 6.0 Hz, 1H), 6.75 (d, J = 6.4 Hz, 1H), 4.56- 4.53 (m, 1H), 4.31- 4.28 (m, 3H), 4.26- 4.25 (m, 1H), 4.23- 4.21 (m, 1H), 3.96- 3.84 (m, 2H), 3.60 (d, J = 4.0 Hz, 2H), 3.36 (s, 3H), 1.46 (s, 9H) |
| tert-butyl (R)-3-(4-(3- methoxypyrrolidin-1- yl)pyridin-3-yl)azetidine- 1-carboxylate | 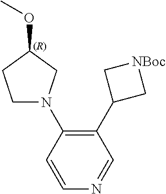 | 334.1 [M + H]⁺ | ¹H NMR (400 MHz, CDCl₃) δ = 8.47 (s, 1H), 8.26 (d, J = 6.0 Hz, 1H), 6.70 (d, J = 6.4 Hz, 1H), 4.34- 4.31 (m, 2H), 4.26- 4.24 (m, 2H), 4.08- 4.01 (m, 2H), 3.67- 3.64 (m, 2H), 3.62- 3.42 (m, 2H), 3.36 (s, 3H), 2.20-2.03 (m, 2H), 1.47 (s, 9H) |

Synthesis of 4-(azetidin-1-yl)-5-(azetidin-3-yl)pyrimidine

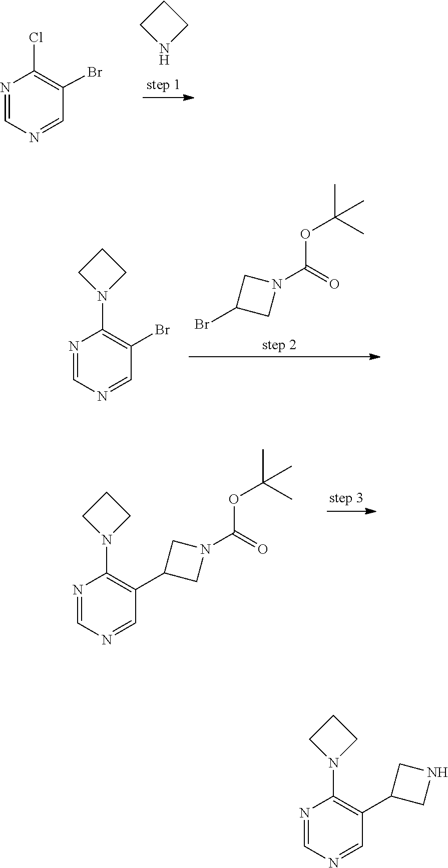

Step 1: Preparation of 4-(azetidin-1-yl)-5-bromopyrimidine

To a solution of 5-bromo-4-chloropyrimidine (2.3 g, 12 mmol, 1.0 eq) in DMA (23 mL) was added azetidine (1.1 g, 12 mmol, 1.0 eq) and caesium carbonate (13 g, 41 mmol, 3.5 eq). The mixture was stirred at 100° C. for 12 hours. The reaction mixture was diluted with H₂O 250 mL and extracted with ethyl acetate 750 mL (250 mL×3). The combined organic layers were washed with sat. brine 750 mL (250 mL×3), dried over anhydrous Na₂SO₄, filtered and concentrated under reduced pressure to give a residue. The residue was purified by flash silica gel chromatography to give 4-(azetidin-1-yl)-5-bromopyrimidine (2.0 g, 9.3 mmol, 79% yield) as a yellow solid. ¹H NMR (400 MHz, CD₃OD) δ 8.32 (s, 1H), 8.19 (s, 1H), 4.44 (s, 4H), 2.41-2.33 (m, 2H).

Step 2: Preparation of tert-butyl 3-(4-(azetidin-1-yl)pyrimidin-5-yl)azetidine-1-carboxylate

To an 15 mL vial equipped with a stir bar was added 4-(azetidin-1-yl)-5-bromopyrimidine (1.0 g, 4.7 mmol, 1.0 eq), tert-butyl 3-bromoazetidine-1-carboxylate (1.4 g, 6.1 mmol, 1.3 eq), Ir [Df (CF₃) ppy]₂ (dtbpy) (PF₆) (52 mg, 47 μmol, 0.01 eq), disodium carbonate (1.0 g, 9.3 mmol, 2.0 eq), TTMSS (49 mg, 1.0 eq), NiCl₂·dtbbpy (28 mg, 70 μmol, 0.015 eq) in DME (10.0 mL). The vial was sealed and placed under nitrogen was added. The reaction was stirred and irradiated with a 10 W blue LED lamp (3 cm away), with cooling water to keep the reaction temperature at 25° C. for 14 hours. The reaction mixture was diluted with H₂O 30 mL and extracted with ethyl acetate 90 mL (30 mL×3). The combined organic layers were washed with sat.brine 90 mL (30 mL×3), dried over anhydrous Na₂SO₄, filtered and concentrated under reduced pressure to give a residue which was purified by flash silica gel chromatography (ISCO®; 40 g SepaFlash® Silica Flash Column, Eluent of 0-7% Ethyl acetate/Methanol gradient @60 mL/min) to give tert-butyl 3-(4-(azetidin-1-yl)pyrimidin-5-yl)azetidine-1-carboxylate (0.25 g, 0.86 mmol, 19% yield) as a yellow solid. ¹H NMR (400 MHz, CD₃OD) δ 8.35 (s, 1H), 8.21 (s, 1H), 4.27 (t, J=7.6 Hz, 6H), 4.02-3.96 (m, 2H), 3.91-3.86 (m, 1H), 2.44-2.36 (m, 2H), 1.45 (s, 9H).

Step 3: Preparation of 4-(azetidin-1-yl)-5-(azetidin-3-yl)pyrimidine

A mixture of tert-butyl 3-(4-(azetidin-1-yl)pyrimidin-5-yl)azetidine-1-carboxylate (0.15 g, 0.52 mmol, 1.0 eq) in DCM (1.5 mL) was added trifluoroacetic acid (0.5 mL), the mixture was stirred at 25° C. for 30 minutes. The reaction mixture was concentrated under reduced pressure to give 4-(azetidin-1-yl)-5-(azetidin-3-yl)pyrimidine (95.0 mg, crude) as a brown oil. LCMS (ESI) m/z=191.1.

Synthesis of 7-(azetidin-3-yl)-1-methyl-1H-pyrrolo[3,2-c]pyridine

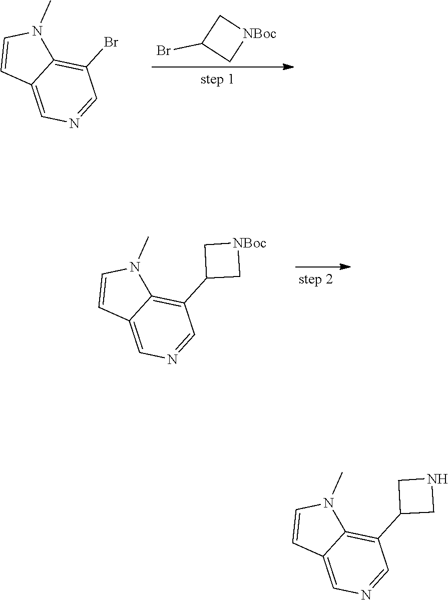 [SMILES: Cn1ccc2cncc(Br)c21 ; CC(C)(C)OC(=O)N1CC(Br)C1 ; Cn1ccc2cncc(C3CN(C(=O)OC(C)(C)C)C3)c21 ; Cn1ccc2cncc(C3CNC3)c21]

Step 1: Preparation of tert-butyl 3-(1-methyl-1H-pyrrolo[3,2-c]pyridin-7-yl)azetidine-1-carboxylate

To an 40 mL vial equipped with a stir bar was added 7-bromo-1-methyl-1H-pyrrolo[3,2-c]pyridine (2.0 g, 9.5 mmol, 1.0 eq), tert-butyl 3-bromoazetidine-1-carboxylate (2.9 g, 12 mmol, 1.3 eq), sodium carbonate (2.0 g, 19 mmol, 2.0 eq), Ir[dF(CF₃)ppy]₂(dtbpy)(PF₆) (0.11 g, 95 μmol, 0.01 eq), NiCl₂·dtbbpy (3.7 g, 9.5 mmol, 1.0 eq), 1,1,1,3,3,3-hexamethyl-2-(trimethylsilyl)trisilane (2.4 g, 9.5 mmol, 1.0 eq) in DCE (40 mL). The vial was sealed and placed under nitrogen was added. The reaction was stirred and irradiated with a 4×50 W [455 nm] blue LED lamp (3 cm away), with cooling water to keep the reaction temperature at 25° C. for 16 hr. The reaction mixture was filtered and concentrated under reduced pressure to give a residue. The residue was purified by flash silica gel chromatography to get tert-butyl 3-{1-methyl-1H-pyrrolo[3,2-c]pyridin-7-yl}azetidine-1-carboxylate (1.0 g, 3.45 mmol, 37% yield) as a off-white solid. ¹H NMR (400 MHz, CDCl₃) δ 9.10 (s, 1H), 8.51 (s, 1H), 7.38 (d, J=3.2 Hz, 1H), 6.98 (d, J=2.0 Hz, 1H), 4.49-4.43 (m, 2H), 4.29-4.19 (m, 2H), 4.12 (s, 3H), 3.78-3.54 (m, 1H), 1.49 (d, J=0.8 Hz, 9H).

Step 2: Preparation of 7-(azetidin-3-yl)-1-methyl-1H-pyrrolo[3,2-c]pyridine

To a solution of tert-butyl 3-{1-methyl-1H-pyrrolo[3,2-c]pyridin-7-yl}azetidine-1-carboxylate (80 mg, 0.28 mmol, 1.0 eq) in DCM (1.0 mL) was added trifluoroacetic acid (0.3 mL), the mixture was stirred at 25° C. for 1 hour. The mixture was concentrated directly to give 3-{1-methyl-1H-pyrrolo[3,2-c]pyridin-7-yl}azetidine (52.0 mg, crude) as a yellow oil. LCMS (ESI) m/z=188.0

Preparation of racemic-trans-6-(pyridin-3-yl)-4-azaspiro[2.4]heptane-7-carbonitrile

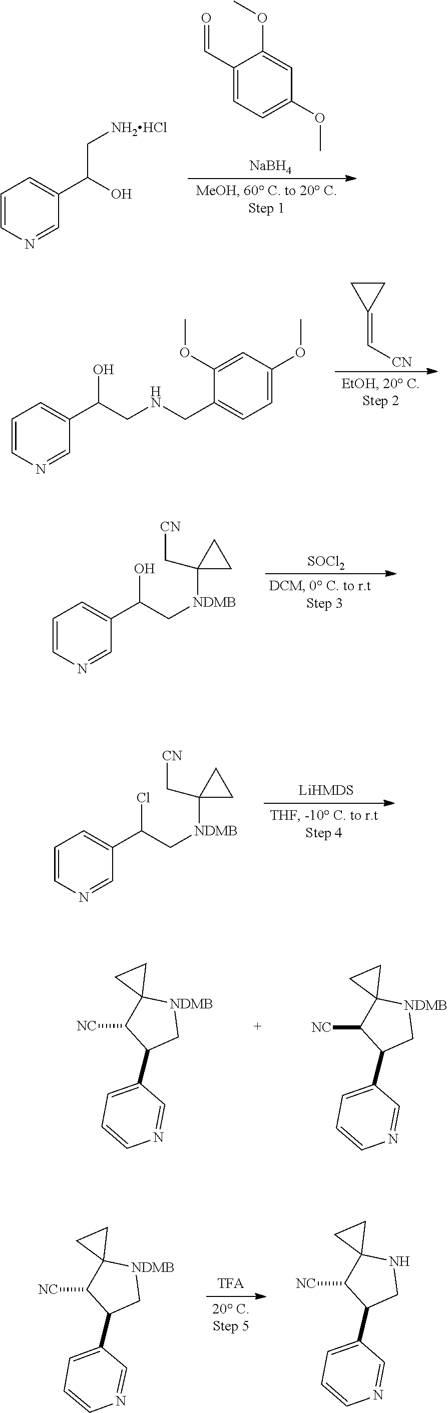

Step 1: Preparation of 2-((2,4-dimethoxybenzyl)amino)-1-(pyridin-3-yl)ethan-1-ol

A solution of 2-amino-1-(pyridin-3-yl)ethan-1-ol dihydrochloride (2.8 g, 13.2 mmol, 1 eq.) and 2,4-dimethoxybenzaldehyde (2.19 g, 13.2 mmol, 1 eq.) in MeOH (10 mL) was stirred at 60° C. for 0.5 h, then NaBH₄ (1.50 g, 39.5 mmol, 3 eq.) was added at 20° C. and the reaction mixture was stirred at 20° C. for 16 h under N₂ atmosphere. After completion, the reaction mixture was poured into ice-NH₄Cl (100 mL), then extracted with EtOAc (50 mL×3). The organic layers were combined and washed with brine (100 mL), dried over anhydrous Na₂SO₄, and concentrated under reduced pressure to afford 2-((2,4-dimethoxybenzyl)amino)-1-(pyridin-3-yl)ethan-1-ol (2.50 g, 8.67 mmol, 66%) as a yellow oil. LCMS (ESI): m/z=289.2 [M+H]⁺. ¹H NMR (400 MHz, DMSO) δ 8.52 (d, J=2.0 Hz, 1H), 8.44 (dd, J=4.8, 1.6 Hz, 1H), 7.71 (dt, J=7.8, 1.8 Hz, 1H), 7.33 (dd, J=7.7, 4.9 Hz, 1H), 7.13 (d, J=8.2 Hz, 1H), 6.52 (d, J=2.3 Hz, 1H), 6.45 (dd, J=8.2, 2.4 Hz, 1H), 5.46 (s, 1H), 4.70 (t, J=6.0 Hz, 1H), 3.74 (d, J=3.2 Hz, 6H), 3.64 (d, J=4.7 Hz, 2H), 3.17 (d, J=5.1 Hz, 1H), 2.70-2.62 (m, 2H).

Step 2: Preparation of 2-(1-((2,4-dimethoxybenzyl)(2-hydroxy-2-(pyridin-3-yl)ethyl)amino)cyclopropyl)acetonitrile

To a mixture of 2-((2,4-dimethoxybenzyl)amino)-1-(pyridin-3-yl)ethan-1-ol (2.5 g, 8.67 mmol, 1 eq.) in EtOH(5 mL) was added 2-cyclopropylideneacetonitrile in PE solution (1.36 g, 17.3 mmol, 2 eq.) and the resulting mixture was stirred at 20° C. for 1 h under N₂ atmosphere. After completion, the reaction mixture was concentrated under reduced pressure to give a residue. The residue was purified by flash column chromatography on silica gel to get 2-(1-((2,4-dimethoxybenzyl)(2-hydroxy-2-(pyridin-3-yl)ethyl)amino)cyclopropyl)acetonitrile (2.40 g, 6.53 mmol, 75%) as a pale-yellow oil. LCMS (ESI): m/z=368.2 [M+H]⁺. ¹H NMR (400 MHz, DMSO) δ 8.40 (dd, J=4.7, 1.6 Hz, 1H), 8.30 (d, J=1.8 Hz, 1H), 7.54 (dt, J=7.8, 1.8 Hz, 1H), 7.28 (dd, J=7.8, 4.8 Hz, 1H), 7.18 (d, J=8.3 Hz, 1H), 6.54 (d, J=2.3 Hz, 1H), 6.47 (dd, J=8.3, 2.4 Hz, 1H), 4.99 (d, J=3.1 Hz, 1H), 4.28 (dd, J=9.8, 6.6 Hz, 1H), 3.85-3.71 (m, 8H), 2.94-2.71 (m, 4H), 0.60-0.31 (m, 3H), 0.06 (s, 1H).

Step 3: Preparation of 2-(1-((2-chloro-2-(pyridin-3-yl)ethyl)(2,4-dimethoxybenzyl)amino)cyclopropyl)acetonitrile

SOCl₂ in DCM solution (3, 281 mg, 2.37 mmol, 1.2 eq.) was added to a solution 2-(1-((2,4-dimethoxybenzyl)(2-hydroxy-2-(pyridin-3-yl)ethyl)amino)cyclopropyl)acetonitrile (730 mg, 1.98 mmol, 1 eq.) in DCM (10 mL) at 0° C. The solution was stirred at room temperature for 0.5 h. After completion, the reaction mixture was quenched with NaHCO₃ (aq., sat.) (20 mL), then extracted with EtOAc (50 mL×2). The organic layers were combined and washed with brine (100 mL), dried over anhydrous Na₂SO₄, and concentrated under reduced pressure. The residue was purified by flash column chromatography on silica gel to afford 2-(1-((2-chloro-2-(pyridin-3-yl)ethyl)(2,4-dimethoxybenzyl)amino)cyclopropyl)acetonitrile (310 mg, 0.80 mmol, 41%) as an oil. LCMS (ESI): m/z=386.2 [M+H]⁺. ¹H NMR (400 MHz, DMSO) δ 8.50 (dd, J=4.8, 1.5 Hz, 1H), 8.31 (d, J=1.9 Hz, 1H), 7.64-7.57 (m, 1H), 7.37 (dd, J=7.9, 4.8 Hz, 1H), 7.13 (d, J=8.3 Hz, 1H), 6.55 (d, J=2.3 Hz, 1H), 6.49 (dd, J=8.3, 2.4 Hz, 1H), 4.62 (t, J=7.2 Hz, 1H), 3.81-3.72 (m, 8H), 3.36-3.23 (m, 2H), 2.91-2.75 (m, 2H), 0.61-0.38 (m, 3H), 0.06-0.04 (m, 1H).

Step 4: Preparation of racemic-trans-4-(2,4-dimethoxybenzyl)-6-(pyridin-3-yl)-4-azaspiro[2.4]heptane-7-carbonitrile & racemic-cis-4-(2,4-dimethoxybenzyl)-6-(pyridin-3-yl)-4-azaspiro[2.4]heptane-7-carbonitrile

To a solution of 2-(1-((2-chloro-2-(pyridin-3-yl)ethyl)(2,4-dimethoxybenzyl)amino)cyclopropyl)acetonitrile (310 mg, 0.80 mmol, 1 eq.) in THF (10 mL) was added LiHMDS (202 mg, 2.00 mmol, 2.5 eq.) dropwise at −10° C. and the solution was stirred at room temperature for 1 hr. After completion, the reaction mixture was quenched with NH₄Cl (aq., sat.) (15 mL), then extracted with EtOAc (15 mL×2). The organic layers were combined and washed with brine (10 mL), dried over anhydrous Na₂SO₄, and concentrated under reduced pressure. The residue was purified by flash column chromatography on silica gel to afford racemic-trans-4-(2,4-dimethoxybenzyl)-6-(pyridin-3-yl)-4-azaspiro[2.4]heptane-7-carbonitrile (75 mg, 0.21 mmol, 27%) and cis-4-(2,4-dimethoxybenzyl)-6-(pyridin-3-yl)-4-azaspiro[2.4]heptane-7-carbonitrile (55.0 mg, 0.16 mmol, 20%) as a yellow oil. LCMS (ESI): m/z=350.2 [M+H]⁺.

Step 5: Preparation of racemic-trans-6-(pyridin-3-yl)-4-azaspiro[2.4]heptane-7-carbonitrile

A solution of racemic-trans-4-(2,4-dimethoxybenzyl)-6-(pyridin-3-yl)-4-azaspiro[2.4]heptane-7-carbonitrile (100 mg, 0.29 mmol, 1 eq.) in TFA (5 mL) was stirred at 20° C. overnight under N₂. After completion, the reaction mixture was quenched with NaHCO₃ (aq., sat.) (20 mL), then extracted with EtOAc (20 mL×2). The organic layers were combined and washed with brine (100 mL), dried over anhydrous Na₂SO₄, and concentrated under reduced pressure to afford racemic-trans-6-(pyridin-3-yl)-4-azaspiro[2.4]heptane-7-carbonitrile (55.0 mg, quant.) as an oil for next step without further purification. LCMS (ESI): m/z=199.9 [M+H]⁺.

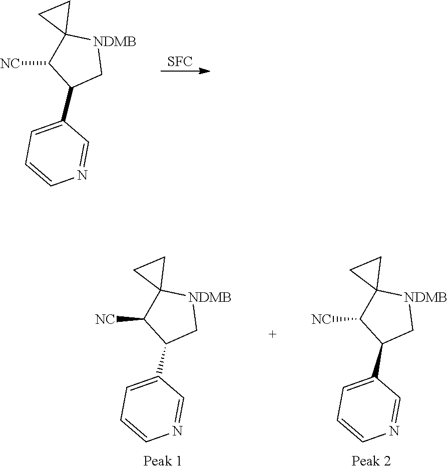

Rac-(6R,7S)-4-(2,4-dimethoxybenzyl)-6-(pyridin-3-yl)-4-azaspiro[2.4]heptane-7-carbonitrile (8 g) was separated by chiral SFC to afford (6S,7R)-4-(2,4-dimethoxybenzyl)-6-(pyridin-3-yl)-4-azaspiro[2.4]heptane-7-carbonitrile (Peak 1, 4.0 g, 50%) and (6R,7S)-4-(3,4-dimethoxybenzyl)-6-(pyridin-3-yl)-4-azaspiro[2.4]heptane-7-carbonitrile (Peak 2, 3.2 g, 40%) as a white solid. Absolute stereochemical configuration was assigned arbitrarily as drawn.

Peak 1: Retention time: 1.946 min; >99% ee.

Peak 2: Retention time: 2.456 min; >99% ee.

Analytical method: Column: ChiralPak IG, 100×4.6 mm I.D., 5 um, Mobile phase: A for CO₂ and B for ethanol(0.05% DEA), Gradient: 8 min @B 30%, Flow rate: 2.5 mL/min, Back pressure: 100 bar, Column temperature: 35° C.

SFC method: Instrument: Waters Thar 80 preparative SFC, Column: ChiralPak IG, 250×21.2 mm I.D., 5 μm, Mobile phase: A for CO₂ and B for EtOH+0.1% NH₃H₂O, Gradient: B 30%, Flow rate: 40 mL/min, Back pressure: 100 bar, Column temperature: 35° C., Wavelength: 220 nm, Cycle-time: 5.2 min, Eluted time: 3H.

The following intermediates in Table 34 were prepared using the method described above in Step 5 for racemic-trans-6-(pyridin-3-yl)-4-azaspiro[2.4]heptane-7-carbonitrile utilizing the appropriate starting materials and modifications.

TABLE 34

| Name | Structure | LCMS |
|---|---|---|
| (6S,7R)-6-(pyridin-3-yl)-4-azaspiro[2.4]heptane-7- carbonitrile | 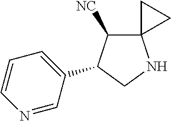 [SMILES: N#C[C@@H]1[C@@H](c2cccnc2)CNC12CC2] | 199.9 [M + H]⁺ |
| (6R,7S)-6-(pyridin-3-yl)-4-azaspiro[2.4]heptane-7- carbonitrile | 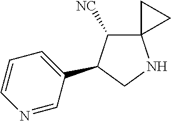 [SMILES: N#C[C@H]1[C@H](c2cccnc2)CNC12CC2] | 199.9 [M + H]⁺ |

Preparation of N,N-dimethyl-3-(4-azaspiro[2.4]heptan-6-yl)pyridin-4-amine

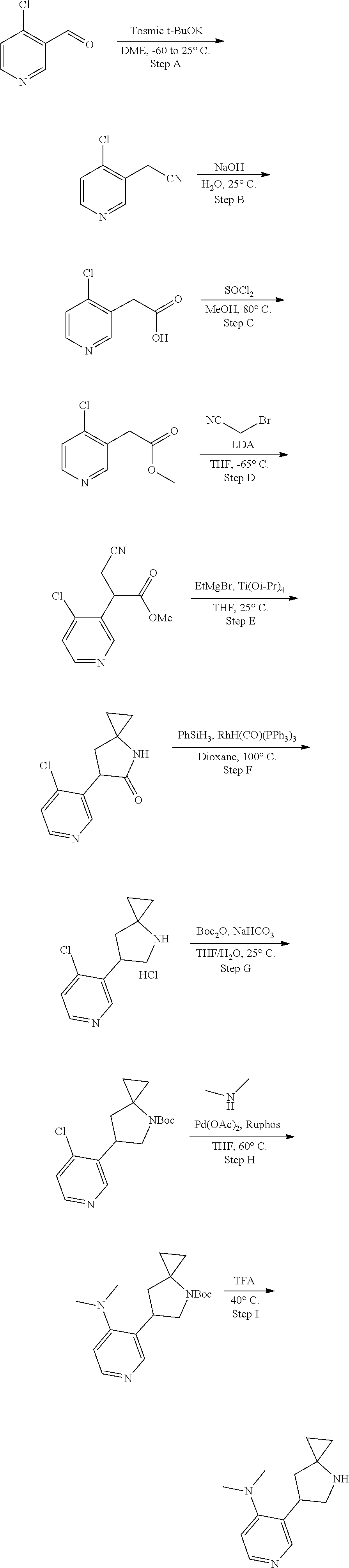

Step A: 2-(4-chloropyridin-3-yl)acetonitrile

To a solution of t-BuOK (15.8 g, 141 mmol, 2 eq.) in DME (100 mL) was added TosMIC (16.5 g, 84.7 mmol, 1.2 eq.) at 25° C. The reaction mixture was cooled to −60° C. 4-chloronicotinaldehyde (10 g, 70.6 mmol, 1 eq.) in DME (100 mL) was added dropwise to the mixture at −60° C. The reaction was stirred at −60° C. for 1 hr, then warmed up to 25° C. and and stirred for 2 hrs under N₂ atmosphere. MeOH (100 mL) was added to the mixture, and the reaction mixture was heated to 80° C. and stirred at 80° C. for 0.5 hrs under N₂ atmosphere. After completion, the reaction mixture was quenched by adding H₂O (200 mL), then extracted with EtOAc (100 mL×3). The organic layers were combined and washed with brine (150 mL), dried over anhydrous Na₂SO₄, and concentrated under reduced pressure. The residue was purified by flash column chromatography on silica gel to afford 2-(4-chloropyridin-3-yl)acetonitrile (4.2 g, 27.5 mmol, 39%) as a yellow solid. LC-MS (ESI) m/z=153 [M+H]⁺.

Step B: 2-(4-chloropyridin-3-yl)acetic acid

2-(4-chloropyridin-3-yl)acetonitrile (4.2 g, 27.5 mmol, 1.0 eq.) was added to a solution of sodium hydroxide (9 g, 225 mmol, 8.2 eq.) in H₂O (51 mL) at 25° C. The reaction mixture was heated to 100° C. and stirred for 1 hr. After completion, the reaction mixture was cooled down in an ice bath, then acidified carefully with HCl (con.) until the pH was adjusted to pH=1. The resulting mixture was dissolved in MeOH (50 mL). The suspension was filtered through a pad of Celite, the filter cake was washed with MeOH (10 mL). The combined filtrates were concentrated under reduced pressure to give 2-(4-chloropyridin-3-yl)acetic acid (4.0 g, 23.3 mmol, 85%) as a brown solid. LC-MS (ESI) m/z=172 [M+H]⁺.

Step C: methyl 2-(4-chloropyridin-3-yl)acetate

To a solution of 2-(4-chloropyridin-3-yl)acetic acid (4 g, 23.3 mmol, 1 eq.) in MeOH (50 mL) was added dropwise thionyl chloride (13.7 g, 116 mmol, 5 eq.) at 25° C. The reaction mixture was heated to 80° C. and stirred for 1 hr. After completion, the reaction mixture was concentrated under reduce pressure. The product was dissolved in water (100 mL), the aqueous phase was neutralized carefully with NaHCO₃ (aq.) until the pH was adjusted to pH=9. The resulting mixture was extracted with EtOAc (100 mL×2), and the combined organic layers were washed with brine (100 mL), dried over with anhydrous Na₂SO₄, then concentrated under reduced pressure. The residue was purified by flash column chromatography on silica gel to afford methyl 2-(4-chloropyridin-3-yl)acetate (4.0 g, 21.5 mmol, 93%) as a yellow oil. LCMS (ESI): m/z=186 [M+H]⁺.

Step D: methyl 2-(4-chloropyridin-3-yl)-3-cyanopropanoate

To a solution of methyl 2-(4-chloropyridin-3-yl)acetate (3 g, 16.1 mmol, 1 eq.) in dry THF (10 mL) was added LDA (9.7 mL, 19.3 mmol, 1.2 eq.) slowly at −65° C. under nitrogen. The mixture was stirred for additional 1 hr at −65° C., then 2-bromoacetonitrile (2.31 g, 19.3 mmol, 1.2 eq.) was added drop-wisely, after addition, the reaction mixture was stirred at −65° C. for 1 hr. After completion, the reaction mixture was quenched by adding sat. NH₄Cl (100 mL), then extracted with EtOAc (100 mL×2). The organic layers were combined and washed with brine (100 mL), dried over anhydrous Na₂SO₄, and concentrated under reduced pressure. The residue was purified by flash column chromatography on silica gel to afford methyl 2-(4-chloropyridin-3-yl)-3-cyanopropanoate (3.0 g, 13.3 mmol, 83%) as a yellow oil. LC-MS (ESI) m/z=225 [M+H]+

Step E: 6-(4-chloropyridin-3-yl)-4-azaspiro[2.4]heptan-5-one

To a solution of methyl 2-(4-chloropyridin-3-yl)-3-cyanopropanoate (1 g, 4.45 mmol, 1 eq.) and titanium isopropoxide (1.58 g, 5.56 mmol, 1.25 eq.) in THF (30 mL) was added dropwise EtMgBr (3.7 mL, 11.1 mmol, 2.5 eq.) at 25° C. for 2 hrs. The reaction mixture was stirred at 25° C. for 0.5 hrs under N₂ atmosphere. After completion, the reaction mixture was diluted with H₂O (10 mL), then extracted with EtOAc (20 mL×3). The organic layers were combined and washed with brine (150 mL), dried over anhydrous Na₂SO₄, and concentrated under reduced pressure. The residue was purified by flash column chromatography on silica gel to afford 6-(4-chloropyridin-3-yl)-4-azaspiro[2.4]heptan-5-one (500 mg, 2.25 mmol, 51%) as a yellow oil. LC-MS (ESI) m/z=223 [M+H]+

Step F: 6-(4-chloropyridin-3-yl)-4-azaspiro[2.4]heptane hydrochloride

To a solution of 6-(4-chloropyridin-3-yl)-4-azaspiro[2.4]heptan-5-one (500 mg, 2.24 mmol, 1.0 eq.) in dioxane (10 mL) were added lambda1-rhodium(1+) formyl radical tris(triphenylphosphine) hydride (103 mg, 112 μmol, 0.05 eq.) and phenylsilane (1.45 g, 13.4 mmol, 6 eq.) at 25° C. The reaction mixture was heated to 100° C. and stirred for 12 hrs under N₂ atmosphere. After completion, the reaction mixture was cooled to 25° C. and acidified carefully with HCl/dioxane (4 M) until the pH was adjusted to pH=1. The resulting mixture was concentrated under reduce pressure. The residue was triturated with H₂O (10 mL) and filtered. The filtrate was concentrate under reduce pressure to give 6-(4-chloropyridin-3-yl)-4-azaspiro[2.4]heptane hydrochloride (200 mg, 815 μmol, 36%) as a yellow solid. LC-MS (ESI) m/z=209 [M+H]⁺.

Step G: tert-butyl 6-(4-chloropyridin-3-yl)-4-azaspiro[2.4]heptane-4-carboxylate

To a solution of 6-(4-chloropyridin-3-yl)-4-azaspiro[2.4]heptane hydrochloride (200 mg, 958 μmol, 1 eq.) and sodium bicarbonate (402 mg, 4.79 mmol, 5 eq.) in a mixture of H₂O (5 mL) and THF (5 mL) was added di-tert-butyl dicarbonate (312 mg, 1.43 mmol, 1.5 eq.) at 25° C. The reaction mixture was stirred at 25° C. for 2 hrs. After completion, the reaction mixture was diluted with H₂O (10 mL), then extracted with EtOAc (20 mL×3). The organic layers were combined and washed with brine (15 mL), dried over anhydrous Na₂SO₄, and concentrated under reduced pressure. The residue was purified by flash column chromatography on silica gel to afford tert-butyl 6-(4-chloropyridin-3-yl)-4-azaspiro[2.4]heptane-4-carboxylate (200 mg, 647 μmol, 68%) as a yellow oil. LC-MS (ESI) m/z=309 [M+H]⁺.

Step H: tert-butyl 6-(4-(dimethylamino)pyridin-3-yl)-4-azaspiro[2.4]heptane-4-carboxylate

To a mixture of tert-butyl 6-(4-chloropyridin-3-yl)-4-azaspiro[2.4]heptane-4-carboxylate (100 mg, 0.32 mmol, 1 eq.), dimethylamine (0.64 mmol, 2.0 eq., 1 M in THF), and t-BuONa (61 mg, 0.64 mmol, 2.0 eq.) in THF(2 mL) were added Pd(OAc)₂ (5 mg, 32 μmol, 0.1 eq.) and Ruphos (30 mg, 64 μmol, 0.2 eq.). The suspension was degassed under vacuum and purged with N₂ several times. The resulting mixture was stirred at 60° C. for 0.5 hrs. After completion, the mixture was concentrated under reduced pressure. The residue was purified by flash column chromatography on silica gel to give tert-butyl 6-(4-(dimethylamino)pyridin-3-yl)-4-azaspiro[2.4]heptane-4-carboxylate (70 mg, 0.22 mmol, 69%) as a yellow oil. LCMS (ESI): m/z=318 [M+H]⁺.

Step I: N,N-dimethyl-3-(4-azaspiro[2.4]heptan-6-yl)pyridin-4-amine

A solution of tert-butyl 6-(4-(dimethylamino)pyridin-3-yl)-4-azaspiro[2.4]heptane-4-carboxylate (100 mg, 0.31 mmol, 1.0 eq.) in TFA (0.5 mL) was stirred at 40° C. for 6 hrs. After completion, the reaction mixture was concentrated under reduced pressure to afford N,N-dimethyl-3-(4-azaspiro[2.4]heptan-6-yl)pyridin-4-amine (100 mg, quant.) (TFA salt) as a brown solid, which was used without further purification. LCMS (ESI): m/z=218 [M+H]⁺.

Preparation of tert-butyl ((3S,6S,7aS,8aR,9aR)-3-((R)-6-(4-(dimethylamino)pyridin-3-yl)-4-azaspiro[2.4]heptane-4-carbonyl)-5-oxodecahydro-1H-cyclopropa[d]pyrrolo[1,2-a]azocin-6-yl)carbamate and tert-butyl ((3S,6S,7aS,8aR,9aR)-3-((S)-6-(4-(dimethylamino)pyridin-3-yl)-4-azaspiro[2.4]heptane-4-carbonyl)-5-oxodecahydro-1H-cyclopropa[d]pyrrolo[1,2-a]azocin-6-yl)carbamate

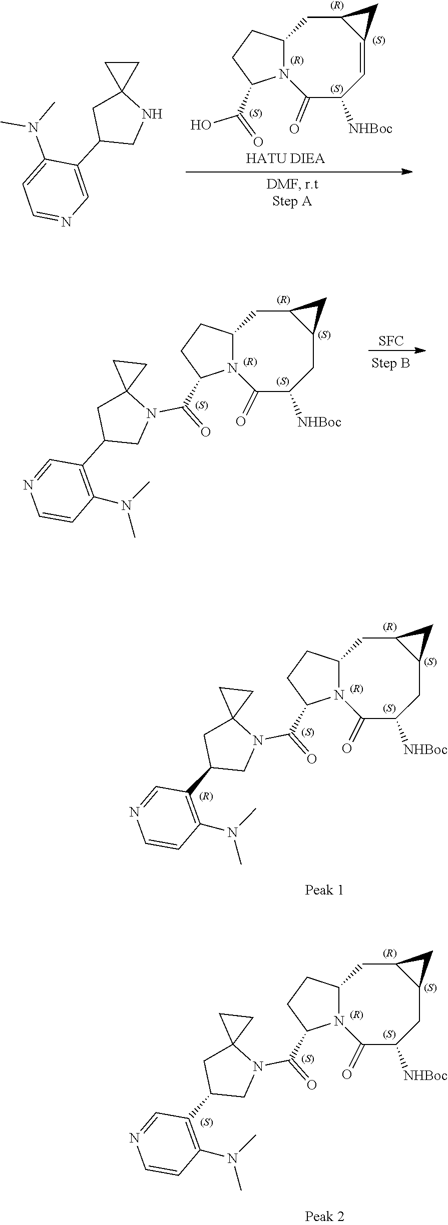

Step A: tert-butyl ((3S,6S,7aS,8aR,9aR)-3-(6-(4-(dimethylamino)pyridin-3-yl)-4-azaspiro[2.4]heptane-4-carbonyl)-5-oxodecahydro-1H-cyclopropa[d]pyrrolo[1,2-a]azocin-6-yl)carbamate

To a mixture of N,N-dimethyl-3-(4-azaspiro[2.4]heptan-6-yl)pyridin-4-amine (100 mg, 0.46 mmol, 1.0 eq.) and (3S,6S,7aS,8aR,9aR)-6-((tert-butoxycarbonyl)amino)-5-oxodecahydro-1H-cyclopropa[d]pyrrolo[1,2-a]azocine-3-carboxylic acid (155 mg, 0.46 mmol, 1.0 eq.) in DMF (2 mL) were added HATU (262 mg, 0.69 mmol, 1.5 eq.) and DIEA (297 mg, 2.3 mmol, 5.0 eq.). The resulting mixture were stirred at room temperature for 2 hrs. After completion, the reaction mixture was purified by C18 column chromatography to give tert-butyl ((3S,6S,7aS,8aR,9aR)-3-(6-(4-(dimethylamino)pyridin-3-yl)-4-azaspiro[2.4]heptane-4-carbonyl)-5-oxodecahydro-1H-cyclopropa[d]pyrrolo[1,2-a]azocin-6-yl)carbamate (50 mg, 93 μmol, 20%) as a white solid. LCMS (ESI): m/z=538 [M+H]⁺; ¹H NMR (400 MHz, CDCl₃): δ 8.63-8.40 (m, 1H), 8.38-8.20 (m, 1H), 6.94-6.74 (m, 1H), 5.62-5.42 (m, 1H), 4.72-4.49 (m, 2H), 4.17-3.68 (m, 3H), 2.81 (d, J=7.2 Hz, 6H), 2.48-1.84 (m, 11H), 1.74-1.46 (m, 3H), 1.44-1.38 (m, 9H), 0.94-0.73 (m, 2H), 0.64-0.41 (m, 2H), −0.04-−0.10 (m, 1H).

Step B: tert-butyl ((3S,6S,7aS,8aR,9aR)-3-((R)-6-(4-(dimethylamino)pyridin-3-yl)-4-azaspiro[2.4]heptane-4-carbonyl)-5-oxodecahydro-1H-cyclopropa[d]pyrrolo[1,2-a]azocin-6-yl)carbamate & tert-butyl ((3S,6S,7aS,8aR,9aR)-3-((S)-6-(4-(dimethylamino)pyridin-3-yl)-4-azaspiro[2.4]heptane-4-carbonyl)-5-oxodecahydro-1H-cyclopropa[d]pyrrolo[1,2-a]azocin-6-yl)carbamate

Tert-butyl ((3S,6S,7aS,8aR,9aR)-3-(6-(4-(dimethylamino)pyridin-3-yl)-4-azaspiro[2.4]heptane-4-carbonyl)-5-oxodecahydro-1H-cyclopropa[d]pyrrolo[1,2-a]azocin-6-yl)carbamate (570 mg) was separated by chiral SFC to afford tert-butyl ((3S,6S,7aS,8aR,9aR)-3-((R)-6-(4-(dimethylamino)pyridin-3-yl)-4-azaspiro[2.4]heptane-4-carbonyl)-5-oxodecahydro-1H-cyclopropa[d]pyrrolo[1,2-a]azocin-6-yl)carbamate (Peak 1, 280 mg, 49%) and tert-butyl ((3S,6S,7aS,8aR,9aR)-3-((S)-6-(4-(dimethylamino)pyridin-3-yl)-4-azaspiro[2.4]heptane-4-carbonyl)-5-oxodecahydro-1H-cyclopropa[d]pyrrolo[1,2-a]azocin-6-yl)carbamate (Peak 2, 200 mg, 35%) as a white solid. Absolute configuration of the pyrrolidine stereocenter was assigned arbitrarily as drawn.

Peak 1: Retention time: 1.080 min; >99% de.

Peak 2: Retention time: 1.876 min; 97% de.

Analytical method: Column: ChiralPak IH, 100×4.6 mm I.D., 5 μm; Mobile phase: A for CO₂ and B for methanol (0.05% DEA); Gradient: 8 min @20% B; Flow rate: 2.5 mL/min; Column temperature: 40° C.

SFC method: Instrument: Waters Thar 80 preparative SFC, Column: ChiralPak IH, 250×21.2 mm I.D., 5 μm, Mobile phase: A for CO₂ and B for MEOH (0.1% 7 mol/L NH₃ in MeOH), Gradient: B 25%, Flow rate: 40 mL/min, Back pressure: 100 bar, Column temperature: 35° C., Wavelength: 220 nm, Cycle-time: 10 min, Eluted time: 3H.

The following compounds in Table 35 were prepared according to the representative procedure described above for the synthesis of isopropyl ((difluoro(2-(((3S,6S,7aS,8aR,9aR)-3-(3-(morpholine-4-carbonyl)azetidine-1-carbonyl)-5-oxodecahydro-1H-cyclopropa[d]pyrrolo[1,2-a]azocin-6-yl)carbamoyl)benzo[b]thiophen-5-yl)methyl)(phenoxy)phosphoryl)glycinate (Example 2) utilizing the appropriate starting materials and modifications.

TABLE 35

| | | | LCMS | |
|---|---|---|---|---|
| Com- | | | [M + | |
| pound | Name | Structure | H]⁺ | NMR |
| 399 | propyl((S)- ((S)-(2- (((3S,6S,7aR, 8aS,9aR)-3- (3-(4- dimethylami- no)pyridin-3- yl)azetidine- 1-carbonyl)- 5- oxodecahydro- 1H- cyclopropa[d] pyrrolo[1,2- a]azocin-6- yl)carbamoyl) benzo[b]thio- phen-5- yl)fluorometh- l)(phenoxy) phosphoryl)- L-alaninate | 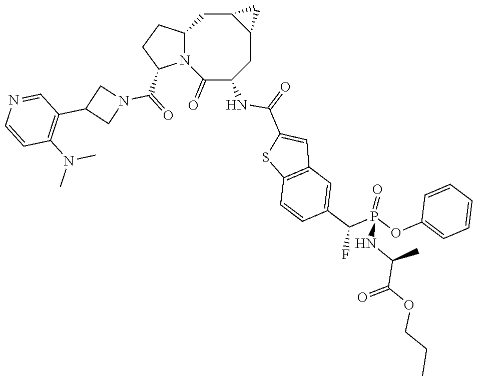 [SMILES: CCCOC(=O)[C@H](C)N[P@@](=O)(Oc1ccccc1)[C@H](F)c1ccc2sc(C(=O)N[C@H]3C[C@H]4C[C@H]4C[C@H]4CC[C@@H](C(=O)N5CC(c6cnccc6N(C)C)C5)N4C3=O)cc2c1] | 859.3 | ¹H NMR (400 MHz, CD₃OD) δ 8.36 (d, J = 17.2 Hz, 1H), 8.16- 8.04 (m, 3H), 8.04- 7.96 (m, 1H), 7.68- 7.62 (m, 1H), 7.37- 7.30 (m, 2H), 7.26- 7.04 (m, 4H), 6.15- 5.99 (m, 1H), 4.95- 4.92 (m, 1H), 4.71 (t, J = 8.4 Hz, 1H), 4.57- 4.47 (m, 1H), 4.45- 4.29 (m, 4H), 4.09- 4.04 (m, 1H), 3.99- 3.95 (m, 2H), 3.83- 3.79 (m, 1H), 3.15 (d, J = 1.6 Hz, 6H), 2.41- 2.32 (m, 1H), 2.28- 2.12 (m, 3H), 2.05- 1.84 (m, 3H), 1.74- 1.51 (m, 3H), 1.46- 1.42 (m, 1H), 1.34- |
| | | | | 1.25 (m, 1H), 1.09- |
| | | | | 1.07 (m, 3H), 0.92- |
| | | | | 0.82 (m, 4H), 0.20- |
| | | | | 0.16 (m, 1H) |
| 398 | propyl((R)- ((S)-(2- (((3S,6S,7aR, 8aS,9aR)-3- (3-(4- (dimethylami- no)pyridin-3- yl)azetidine- 1-carbonyl)- 5- oxodecahydro- 1H- cyclopropa[d] pyrrolo[1,2- a]azocin-6- yl)carbamoyl) benzo[b]thio- phen-5- yl)fluorometh- yl)(phenoxy) phosphoryl)- L-ananinate | 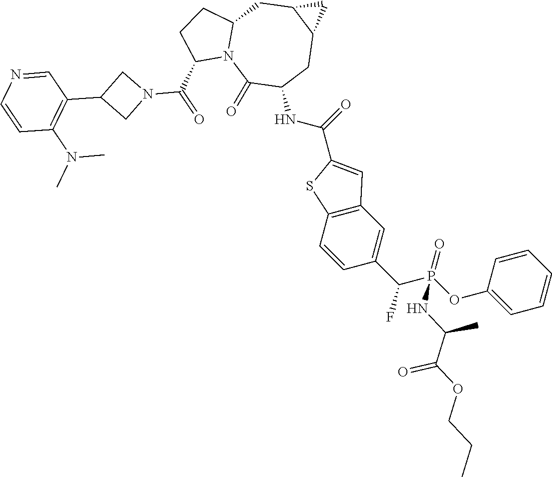 [SMILES: CCCOC(=O)[C@H](C)N[P@@](=O)(Oc1ccccc1)[C@H](F)c1ccc2sc(C(=O)N[C@H]3C[C@H]4C[C@H]4C[C@H]4CC[C@@H](C(=O)N5CC(c6cnccc6N(C)C)C5)N4C3=O)cc2c1] | 859.6 | ¹H NMR (400 MHz, CD₃OD) δ 8.35 (d, J = 14.0, 1H), 8.15-8.06 (m, 3H), 7.98-7.94 (m, 1H), 7.63 (d, J = 8.4 Hz, 1H), 7.36- 7.32 (m, 2H), 7.25- 7.15 (m, 3H), 7.15- 7.04 (m, 1H), 6.19- 6.03 (m, 1H), 4.95- 4.92 (m, 1H), 4.75- 4.67 (m, 1H), 4.57- 4.38 (m, 3H), 4.36- 4.30 (m, 2H), 4.09- 3.86 (m, 4H), 3.15 (d, J = 2.4 Hz, 6H), 2.42- 2.11 (m, 4H), 2.04- 1.83 (m, 3H), 1.72- 1.62 (m, 1H), 1.59- 1.50 (m, 2H), 1.47- 1.38 (m, 1H), 1.33- 1.27 (m, 1H), 1.24 (d, J = 6.8 Hz, 3H), 0.91- 0.84 (m, 4H), 0.20- 0.16 (m, 1H) |
| 397 | propyl(((2- (((3S,6S,7aR, 8aS,9aR)-3- (3-(4- (dimethylami- no)pyridin-3- yl)azetidine- 1-carbonyl)- 5- oxodecahydro- 1H- cyclopropa[d] pyrrolo[1,2- a]azocin-6- yl)carbamoyl) benzo[b]thio- phen-5- yl)methyl)(phe- noxy)phos- | 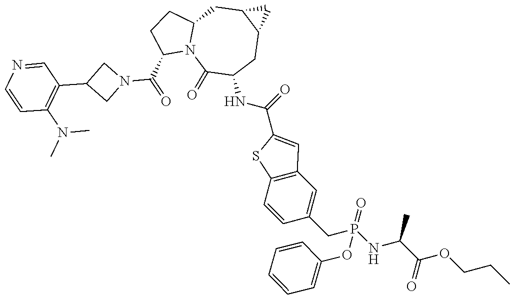 [SMILES: CCCOC(=O)[C@H](C)NP(=O)(Cc1ccc2sc(C(=O)N[C@H]3C[C@H]4C[C@H]4C[C@H]4CC[C@@H](C(=O)N5CC(c6cnccc6N(C)C)C5)N4C3=O)cc2c1)Oc1ccccc1] | 841.3 | ¹H NMR (400 MHz, CD₃OD) δ 8.39-8.33 (m, 1H), 8.16-8.00 (m, 2H), 7.93-7.85 (m, 2H), 7.52-7.46 (m, 1H), 7.36-7.29 (m, 2H), 7.20-7.06 (m, 4H), 4.94-4.86 (m, 1H), 4.71-4.69 (m, 1H), 4.57-4.47 (m, 1H), 4.42-4.39 (m, 2H), 4.36-4.31 (m, 2H), 4.09-4.02 (m, 1H), 3.95-3.82 (m, 3H), 3.58-3.48 (m, 2H), 3.15 (s, 6H), 2.39-2.14 (m, 4H), 2.02-1.85 (m, 3H), 1.74-1.61 (m, 1H), |
| | phoryl)-L- | | | 1.58-1.50 (m, 2H), |
| | alaninate | | | 1.46-1.40 (m, 1H), |
| | | | | 1.34-1.27 (m, 1H), |
| | | | | 1.15 (d, J = 7.2 Hz, |
| | | | | 1H), 1.12-1.06 (m, |
| | | | | 2H), 0.90-0.84 (m, |
| | | | | 4H), 0.19-0.16 (m, |
| | | | | 1H) |
| 396 | propyl((S)- ((S)- fluoro(2- (((3S,6S,7aR, 8aS,9aR)-3- (3-(4- morpholinopy- ridin-3- yl)azetidine- 1-carbonyl)- 5- oxodecahydro- 1H- cyclopropa[d] pyrrolo[1,2- a]azocin-6- yl)carbamoyl) benzo[b]thio- phen-5- yl)methyl)(phe- noxy)phos- phoryl)-L- alaninate | 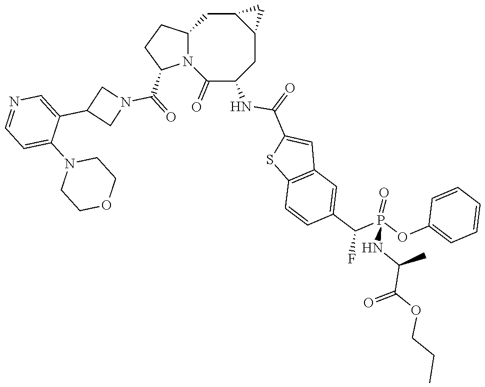 [SMILES: CCCOC(=O)[C@H](C)N[P@@](=O)(Oc1ccccc1)[C@H](F)c1ccc2sc(C(=O)N[C@H]3C[C@H]4C[C@H]4C[C@H]4CC[C@@H](C(=O)N5CC(c6cnccc6N6CCOCC6)C5)N4C3=O)cc2c1] | 901.2 | ¹H NMR (400 MHz, CDCl₃) δ 9.13-8.64 (m, 1H), 8.48 (dd, J = 6.4, 19.2 Hz, 1H), 8.36-8.02 (m, 1H), 7.97 (s, 1H), 7.91- 7.80 (m, 1H), 7.73- 7.53 (m, 1H), 7.29- 7.27 (m, 1H), 7.27- 7.22 (m, 2H), 7.11 (q, J = 6.4 Hz, 3H), 6.10- 5.81 (m, 1H), 5.12- 4.99 (m, 1H), 4.97- 4.78 (m, 1H), 4.73- 4.63 (m, 1H), 4.40- 4.34 (m, 1H), 4.32- 4.25 (m, 1H), 4.24- 4.19 (m, 1H), 4.19- 4.07 (m, 2H), 4.00 (s, 2H), 3.91-3.84 (m, 4H), 3.71-3.59 (m, 1H), 3.36-3.16 (m, 4H), 2.48-2.20 (m, 2H), 2.15-2.05 (m, |
| | | | | 3H), 2.05-1.94 (m, |
| | | | | 2H), 1.76-1.67 (m, |
| | | | | 1H), 1.67-1.60 (m, |
| | | | | 2H), 1.57-1.43 (m, |
| | | | | 1H), 1.31 (t, J = 6.8 |
| | | | | Hz, 3H), 1.25-1.15 |
| | | | | (m, 1H), 0.91 (q, J = |
| | | | | 7.2 Hz, 3H), 0.86- |
| | | | | 0.78 (m, 1H), 0.27- |
| | | | | 0.17 (m, 1H) |
| 395 | propyl((R)- ((S)- fluoro(2- (((3S,6S,7aR, 8aS,9aR)-3- (3-(4- morpholinopy- ridin-3- yl)azetidine- 1-carbonyl)- 5- oxodecahydro- 1H- cyclopropa[d] pyrrolo[1,2- a]azocin-6- yl)carbamoyl) benzo[b]thio- phen-5- yl)methyl)(phe- noxy)phos- phoryl)-L- alaninate | 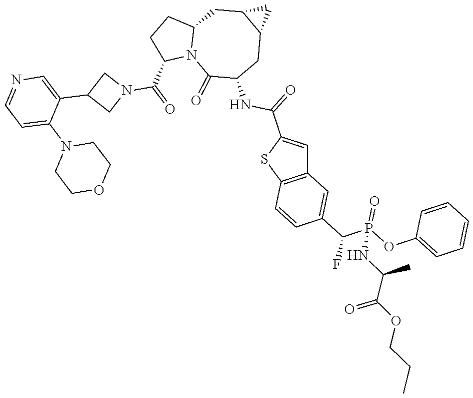 [SMILES: CCCOC(=O)[C@H](C)N[P@](=O)(Oc1ccccc1)[C@H](F)c1ccc2sc(C(=O)N[C@H]3C[C@H]4C[C@H]4C[C@H]4CC[C@@H](C(=O)N5CC(c6cnccc6N6CCOCC6)C5)N4C3=O)cc2c1] | 901.2 | ¹H NMR (400 MHz, CDCl₃) δ 9.10-8.67 (m, 1H), 8.48 (t, J = 5.6 Hz, 1H), 8.38- 8.06 (m, 1H), 7.98 (s, 1H), 7.89-7.79 (m, 1H), 7.70-7.52 (m, 1H), 7.43-7.30 (m, 2H), 7.29-7.27 (m, 1H), 7.22 (d, J = 3.2 Hz, 1H), 7.21-7.16 (m, 1H), 7.14-7.03 (m, 1H), 6.09-5.88 (m, 1H), 5.13-5.01 (m, 1H), 4.98-4.82 (m, 1H), 4.75-4.62 (m, 1H), 4.41-4.36 (m, 1H), 4.33-4.19 (m, 2H), 4.17-4.07 (m, 1H), 4.04-3.94 (m, 2H), 3.93-3.87 (m, 4H), 3.76 (s, 1H), 3.65 (s, 1H), 3.28- 3.15 (m, 4H), 2.49- |
| | | | | 2.40 (m, 1H), 2.37- |
| | | | | 2.29 (m, 1H), 2.29- |
| | | | | 2.20 (m, 1H), 2.12 |
| | | | | (dd, J = 6.4, 12.4 Hz, |
| | | | | 3H), 2.03-1.95 (m, |
| | | | | 2H), 1.73-1.64 (m, |
| | | | | 1H), 1.54 (t, J = 7.2 |
| | | | | Hz, 3H), 1.31-1.21 |
| | | | | (m, 4H), 0.86 (t, J = |
| | | | | 3.6 Hz, 3H), 0.30- |
| | | | | 0.14 (m, 1H) |
| 392 | propyl(((2- (((3S,6S,7aR, 8aS,9aR)-3- (3-(4- ethylpyridin- 3- yl)azetidine- 1-carbonyl)- 5- oxodecahydro- 1H- cyclopropa[d] pyrrolo[1,2- a]azocin-6- yl)carbamoyl) benzo[b]thio- phen-5- | 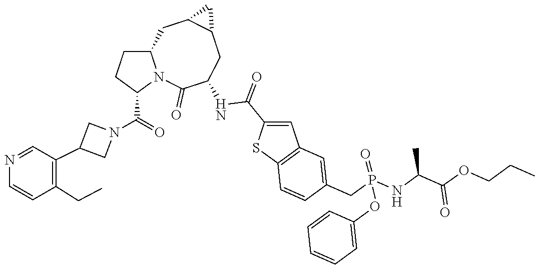 [SMILES: CCCOC(=O)[C@H](C)NP(=O)(Cc1ccc2sc(C(=O)N[C@H]3C[C@H]4C[C@H]4C[C@H]4CC[C@@H](C(=O)N5CC(c6cnccc6CC)C5)N4C3=O)cc2c1)Oc1ccccc1] | 826.4 | ¹H NMR (400 MHz, CD₃OD) δ 8.94-8.84 (m, 1H), 8.73-8.51 (m, 1H), 8.02 (d, J = 18.8 Hz, 1H), 7.95- 7.78 (m, 3H), 7.52- 7.43 (m, 1H), 7.37- 7.25 (m, 2H), 7.22- 7.11 (m, 3H), 4.96- 4.92 (m, 1H), 4.82- 4.73 (m, 1H), 4.66- 4.50 (m, 1H), 4.49- 4.40 (m, 2H), 4.39- 4.31 (m, 2H), 4.19- 4.10 (m, 1H), 3.97- 3.89 (m, 2H), 3.80 (s, 1H), 3.58-3.44 (m, |
| | yl)methyl)(phe- | | | 2H), 2.90-2.78 (m, |
| | noxy)phos- | | | 2H), 2.43-2.33 (m, |
| | phoryl)-L- | | | 1H), 2.32-2.23 (m, |
| | alaninate | | | 1H), 2.23-2.12 (m, |
| | | | | 2H), 2.04-1.93 (m, |
| | | | | 2H), 1.93-1.84 (m, |
| | | | | 1H), 1.74-1.62 (m, |
| | | | | 1H), 1.59-1.49 (m, |
| | | | | 2H), 1.48-1.40 (m, |
| | | | | 1H), 1.32 (s, 1H), |
| | | | | 1.31-1.24 (m, 3H), |
| | | | | 1.18-1.06 (m, 3H), |
| | | | | 0.91-0.88 (m, 1H), |
| | | | | 0.88-0.79 (m, 3H), |
| | | | | 0.82-0.08 (m, 1H) |
| 382 | propyl(((2- (((3S,6S,7aS, 8aR,9aR)-3- (((3S,4S)-1- carbamoyl-4- fluoropyrroli- din-3- yl)carbamoyl)- 5- oxodecahydro- 1H- cyclopropa[d] pyrrolo[1,2- a]azocin-6- yl)carbamoyl) benzo[b]thio- phen-5- yl)fluorometh- yl)(phenoxy) phosphoryl)- L-alaninate | 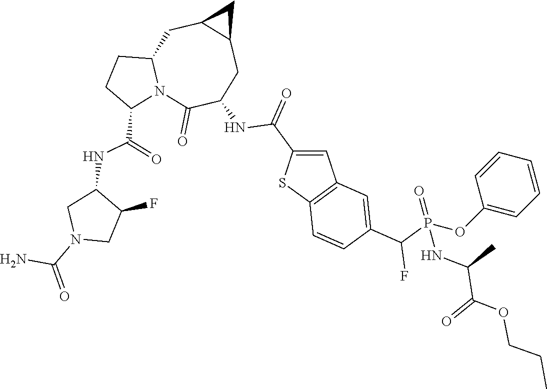 [SMILES: CCCOC(=O)[C@H](C)NP(=O)(Oc1ccccc1)C(F)c1ccc2sc(C(=O)N[C@H]3C[C@@H]4C[C@@H]4C[C@H]4CC[C@@H](C(=O)N[C@H]5CN(C(N)=O)C[C@@H]5F)N4C3=O)cc2c1] | 829.2 | ¹H NMR (400 MHz, CDCl₃) δ 8.28-8.04 (m, 1H), 8.01-7.95 (m, 1H), 7.93-7.83 (m, 2H), 7.78-7.64 (m, 1H), 7.62-7.50 (m, 1H), 7.39-7.30 (m, 1H), 7.28-7.17 (m, 3H), 7.14-7.00 (m, 2H), 6.08-5.86 (m, 1H), 5.62-5.33 (m, 1H), 5.15-4.90 (m, 2H), 4.68-4.58 (m, 1H), 4.47 (d, J = 1.6 Hz, 1H), 4.19 (d, J = 7.6 Hz, 1H), 4.14- 3.94 (m, 3H), 3.92- 3.71 (m, 2H), 3.68- 3.54 (m, 2H), 3.36 (d, J = 9.6 Hz, 1H), 2.38- 2.22 (m, 3H), 2.19- 2.07 (m, 2H), 2.05- 1.97 (m, 1H), 1.93- 1.85 (m, 1H), 1.67- |
| | | | | 1.50 (m, 3H), 1.35- |
| | | | | 1.12 (m, 3H), 1.06- |
| | | | | 0.66 (m, 6H), 0.05 (s, |
| | | | | 1H) |
| 381 | propyl(((2- (((3S,6S,7aS, 8aR,9aR)-3- (3-(5- (difluorometh- yl)isoxazol- 3- yl)azetidine- 1-carbonyl)- 5- oxodecahydro- 1H- cyclopropa[d] pyrrolo[1,2- a]azocin-6- yl)carbamoyl) benzo[b]thio- phen-5- yl)fluorometh- | 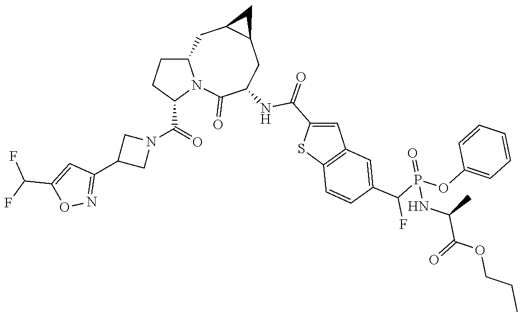 [SMILES: CCCOC(=O)[C@H](C)NP(=O)(Oc1ccccc1)C(F)c1ccc2sc(C(=O)N[C@H]3C[C@@H]4C[C@@H]4C[C@H]4CC[C@@H](C(=O)N5CC(c6cc(C(F)F)on6)C5)N4C3=O)cc2c1] | 856.4 | ¹H NMR (400 MHz, CDCl₃) δ 8.63-8.61 (m, 1H), 7.98-7.95 (m, 1H), 7.85-7.77 (m, 2H), 7.75-7.45 (m, 2H), 7.39-7.34 (m, 1H), 7.25-7.23 (m, 3H), 7.17- 7.04 (m, 2H), 6.86- 6.55 (m, 1H), 6.01- 5.90 (m, 1H), 5.08- 5.02 (m, 2H), 4.56- 4.55 (m, 1H), 4.46- 4.44 (m, 1H), 4.30- 4.22 (m, 2H), 4.10- 4.03 (m, 3H), 3.85- 3.80 (m, 1H), 3.72- 3.61 (m, 1H), 2.36- 2.30 (m, 3H), 2.04- |
| | yl)(phenoxy) | | | 2.02 (m, 4H), 1.63- |
| | phosphoryl)- | | | 1.61 (m, 3H), 1.56- |
| | L-alaninate | | | 1.41 (m, 1H), 1.30- |
| | | | | 1.27 (m, 3H), 0.93- |
| | | | | 0.84 (m, 5H), 0.03- |
| | | | | 0.01 (m, 1H) |
| 363 | propyl ((fluoro(2- (((3S,6S,7aS, 8aR,9aR)-3- (3- ((methylsulfo- nyl)methyl)a- zetidine-1- carbonyl)-5- oxodecahydro- 1H- cyclopropa[d] pyrrolo[1,2- a]azocin-6- yl)carbamoyl) | 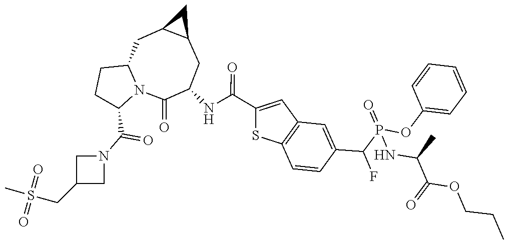 [SMILES: CCCOC(=O)[C@H](C)NP(=O)(Oc1ccccc1)C(F)c1ccc2sc(C(=O)N[C@H]3C[C@@H]4C[C@@H]4C[C@H]4CC[C@@H](C(=O)N5CC(CS(C)(=O)=O)C5)N4C3=O)cc2c1] | 831.6 | ¹H NMR (400 MHz, CD₃OD) δ 8.18-8.05 (m, 2H), 8.02-7.92 (m, 1H), 7.68-7.60 (m, 1H), 7.40-7.29 (m, 2H), 7.26-7.10 (m, 3H), 6.20-5.98 (m, 1H), 5.08-4.98 (m, 1H), 4.81-4.71 (m, 1H), 4.51-4.38 (m, 2H), 4.32-4.22 (m, 1H), 4.20-4.09 (m, 1H), 4.01-3.75 (m, 4H), 3.57-3.43 (m, 2H), 3.28-3.17 |
| | benzo[b]thio- | | | (m, 1H), 2.96-2.95 |
| | phen-5- | | | (m, 3H), 2.51-2.37 |
| | yl)methyl)(phe- | | | (m, 1H), 2.30-2.27 |
| | noxy)phos- | | | (m, 1H), 2.25-2.04 |
| | phoryl)-L- | | | (m, 3H), 2.02-1.89 |
| | alaninate | | | (m, 2H), 1.81-1.66 |
| | | | | (m, 1H), 1.62-1.47 |
| | | | | (m, 2H), 1.45-1.35 |
| | | | | (m, 1H), 1.26-1.18 |
| | | | | (m, 2H), 1.11-1.01 |
| | | | | (m, 1H), 0.99-0.93 |
| | | | | (m, 1H), 0.92-0.79 |
| | | | | (m, 4H), 0.11-−0.01 |
| | | | | (m, 1H) |
| 362 | propyl ((fluoro(2- (((3S,6S,7aS, 8aR,9aR)-3- (3-(3- methoxyisox- azol-5- yl)azetidine- 1-carbonyl)- 5- oxodecahydro- 1H- cyclopropa[d] pyrrolo[1,2- a]azocin-6- yl)carbamoyl) benzo[b]thio- phen-5- | 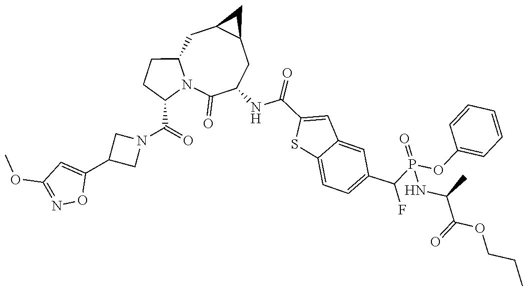 [SMILES: CCCOC(=O)[C@H](C)NP(=O)(Oc1ccccc1)C(F)c1ccc2sc(C(=O)N[C@H]3C[C@@H]4C[C@@H]4C[C@H]4CC[C@@H](C(=O)N5CC(c6cc(OC)no6)C5)N4C3=O)cc2c1] | 836.2 | ¹H NMR (400 MHz, CD₃OD) δ 8.17-8.06 (m, 2H), 8.03-7.93 (m, 1H), 7.69-7.60 (m, 1H), 7.38-7.30 (m, 2H), 7.25-7.13 (m, 3H), 6.18-5.99 (m, 2H), 5.05-5.01 (m, 1H), 4.72-4.61 (m, 1H), 4.54-4.47 (m, 1H), 4.43-4.33 (m, 1H), 4.31-4.27 (m, 1H), 3.93-3.88 (m, 9H), 2.51-2.39 (m, 1H), 2.38-2.07 (m, 5H), 2.02-1.92 (m, 2H), 1.79-1.68 (m, 1H), 1.60-1.50 |
| | yl)methyl)(phe- | | | (m, 2H), 1.41 (s, 1H), |
| | noxy)phos- | | | 1.26-1.18 (m, 2H), |
| | phoryl)-L- | | | 1.09-1.04 (m, J = 7.2 |
| | alaninate | | | Hz, 1H), 1.03-0.80 |
| | | | | (m, 6H), 0.09-0.04 |
| | | | | (m, 1H) |
| 361 | propyl ((fluoro(2- (((3S,6S,7aS, 8aR,9aR)-3- (3-(3- methylisoxazol- 5- yl)azetidine- 1-carbonyl)- 5- oxodecahydro- 1H- cyclopropa[d] pyrrolo[1,2- a]azocin-6- yl)carbamoyl) benzo[b]thio- phen-5- | 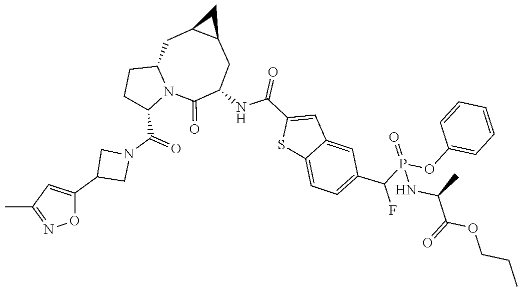 [SMILES: CCCOC(=O)[C@H](C)NP(=O)(Oc1ccccc1)C(F)c1ccc2sc(C(=O)N[C@H]3C[C@@H]4C[C@@H]4C[C@H]4CC[C@@H](C(=O)N5CC(c6cc(C)no6)C5)N4C3=O)cc2c1] | 820.2 | ¹H NMR (400 MHz, CD₃OD) δ 8.17-8.05 (m, 2H), 8.03-7.91 (m, 1H), 7.65 (d, J = 8.2 Hz, 1H), 7.40- 7.29 (m, 2H), 7.27- 7.13 (m, 3H), 6.35- 6.24 (m, 1H), 6.20- 5.99 (m, 1H), 5.04 (t, J = 8.4 Hz, 1H), 4.72- 4.63 (m, 1H), 4.57- 4.48 (m, 1H), 4.45- 4.23 (m, 3H), 4.14- 4.02 (m, 2H), 4.00- 3.94 (m, 1H), 3.93- 3.76 (m, 2H), 2.52- 2.40 (m, 1H), 2.34- 2.12 (m, 7H), 2.06- |
| | yl)methyl)(phe- | | | 1.91 (m, 2H), 1.80- |
| | noxy)phos- | | | 1.68 (m, 1H), 1.63- |
| | phoryl)-L- | | | 1.48 (m, 2H), 1.47- |
| | alaninate | | | 1.36 (m, 1H), 1.27- |
| | | | | 1.19 (m, 2H), 1.10- |
| | | | | 1.04 (m, 1H), 0.99- |
| | | | | 0.80 (m, 5H), 0.12- |
| | | | | 0.02 (m, 1H) |
| 360 | propyl ((fluoro(2- (((3S,6S,7aS, 8aR,9aR)-3- (3-(3-methyl- 1,2,4- oxadiazol-5- yl)azetidine- 1-carbonyl)- 5- oxodecahydro- 1H- cyclopropa[d] pyrrolo[1,2- a]azocin-6- yl)carbamoyl) benzo[b]thio- phen-5- | 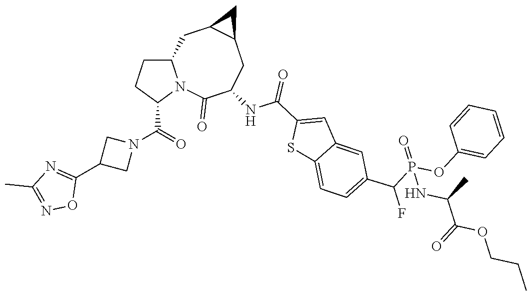 [SMILES: CCCOC(=O)[C@H](C)NP(=O)(Oc1ccccc1)C(F)c1ccc2sc(C(=O)N[C@H]3C[C@@H]4C[C@@H]4C[C@H]4CC[C@@H](C(=O)N5CC(c6nc(C)no6)C5)N4C3=O)cc2c1] | 821.6 | ¹H NMR (400 MHz, CD₃OD) δ 8.16-8.03 (m, 2H), 8.01-7.89 (m, 1H), 7.68-7.69 (m, 1H), 7.39-7.29 (m, 2H), 7.26-7.13 (m, 3H), 6.19-5.98 (m, 1H), 5.07-5.00 (m, 1H), 4.83-4.66 (m, 1H), 4.54-4.42 (m, 2H), 4.40-4.27 (m, 2H), 4.26-4.10 (m, 2H), 4.03-3.75 (m, 3H), 2.56-2.35 (m, 3H), 2.35-2.07 (m, 5H), 2.04-1.91 (m, 2H), 1.79-1.67 (m, 1H), 1.63-1.46 |
| | yl)methyl)(phe- | | | (m, 2H), 1.46-1.34 |
| | noxy)phos- | | | (m, 1H), 1.27-1.18 |
| | phoryl)-L- | | | (m, 2H), 1.049- |
| | alaninate | | | 1.071 (m, 1H), 1.02- |
| | | | | 0.80 (m, 5H), 0.036- |
| | | | | 0.083 (m, 1H) |
| 359 | propyl ((fluoro(2- (((3S,6S,7aS, 8aR,9aR)-3- (3-(5- methylisoxazol- 3- yl)azetidine- 1-carbonyl)- 5- oxodecahydro- 1H- cyclopropa[d] pyrrolo[1,2- a]azocin-6- yl)carbamoyl) benzo[b]thio- phen-5- yl)methyl)(phe- | 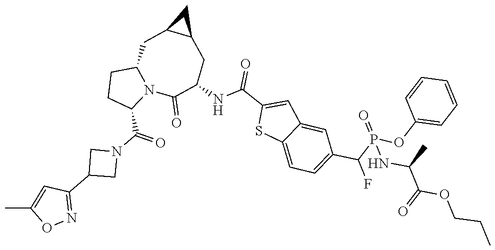 [SMILES: CCCOC(=O)[C@H](C)NP(=O)(Oc1ccccc1)C(F)c1ccc2sc(C(=O)N[C@H]3C[C@@H]4C[C@@H]4C[C@H]4CC[C@@H](C(=O)N5CC(c6cc(C)on6)C5)N4C3=O)cc2c1] | 820.2 | ¹H NMR (400 MHz, CD₃OD) δ 8.21-8.03 (m, 2H), 8.01-7.87 (m, 1H), 7.69-7.57 (m, 1H), 7.42-7.26 (m, 2H), 7.24-7.12 (m, 3H), 6.32-6.18 (m, 1H), 6.17-5.98 (m, 1H), 5.10-5.00 (m, 1H), 4.69-4.61 (m, 1H), 4.57-4.47 (m, 1H), 4.47-4.18 (m, 3H), 4.13-3.74 (m, 5H), 2.50-2.43 (m, 1H), 2.42-2.32 (m, 3H), 2.32-2.27 (m, 1H), 2.26-2.07 (m, 3H), 2.04-1.90 (m, 2H), 1.80-1.67 |
| | noxy)phos- | | | (m, 1H), 1.62-1.47 |
| | phoryl)-L- | | | (m, 2H), 1.46-1.33 |
| | alaninate | | | (m, 1H), 1.25-1.04 |
| | | | | (m, 3H), 1.00-0.92 |
| | | | | (m, 1H), 0.91-0.80 |
| | | | | (m, 4H), 0.10-0.02 |
| | | | | (m, 1H) |
| 338 | propyl ((fluoro(2- (((3S,6S,7aR, 8aS,9aR)-5- oxo-3-(3- (pyrimidin-2- yl)azetidine- 1- carbonyl)deca- hydro-1H- cyclopropa[d] pyrrolo[1,2- a]azocin-6- yl)carbamoyl) benzo[b]thio- | 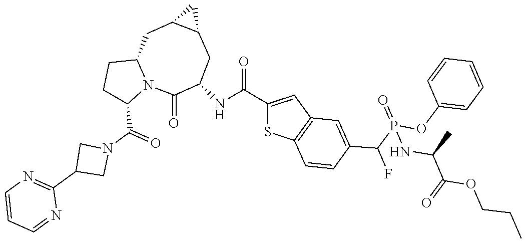 [SMILES: CCCOC(=O)[C@H](C)NP(=O)(Oc1ccccc1)C(F)c1ccc2sc(C(=O)N[C@H]3C[C@H]4C[C@H]4C[C@H]4CC[C@@H](C(=O)N5CC(c6ncccn6)C5)N4C3=O)cc2c1] | 817.5 | ¹H NMR (400 MHz, CDCl₃) δ 8.74-8.82 (m, 2H), 7.92-8.01 (m, 1H), 7.88 (t, J = 8.00 Hz, 1H), 7.74- 7.83 (m, 2H), 7.53- 7.63 (m, 1H), 7.33- 7.40 (m, 1H), 7.19- 7.23 (m, 2H), 7.04- 7.15 (m, 2H), 5.89- 6.09 (m, 1H), 4.83- 4.98 (m, 2H), 4.38- 4.67 (m, 3H), 4.14- 4.30 (m, 3H), 3.97- 4.10 (m, 2H), 3.54- |
| | phen-5- | | | 3.75 (m, 1H), 2.05- |
| | yl)methyl)(phe- | | | 2.13 (m, 3H), 1.96 |
| | noxy)phos- | | | (d, J = 6.00 Hz, |
| | phoryl)-L- | | | 2H), 1.76- |
| | alaninate | | | 1.88 (m, 1H), 1.49- |
| | | | | 1.71 (m, 4H), 1.19- |
| | | | | 1.36 (m, 4H), 1.09- |
| | | | | 1.16 (m, 1H), 0.78- |
| | | | | 0.97 (m, 4H), 0.19- |
| | | | | 0.25 (m, 1H) |
| 332 | propyl ((fluoro(2- (((3S,6S,7aR, 8aS,9aR)-3- (3-(4-methyl- 4H-1,2,4- triazol-3- yl)azetidine- 1-carbonyl)- 5- oxodecahydro- 1H- cyclopropa[d] pyrrolo[1,2- a]azocin-6- | 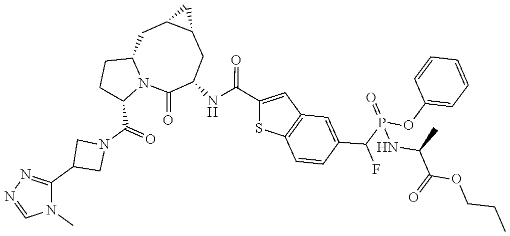 [SMILES: CCCOC(=O)[C@H](C)NP(=O)(Oc1ccccc1)C(F)c1ccc2sc(C(=O)N[C@H]3C[C@H]4C[C@H]4C[C@H]4CC[C@@H](C(=O)N5CC(c6nncn6C)C5)N4C3=O)cc2c1] | 820.4 | ¹H NMR (400 MHz, DMSO-d₆) δ 8.94- 8.81 (m, 1H), 8.44- 8.37 (m, 1H), 8.29 (d, J = 7.2 Hz, 1H), 8.07 (s, 2H), 7.61 (s, 1H), 7.38-7.31 (m, 2H), 7.23-7.09 (m, 3H), 6.29-6.07 (m, 2H), 4.84-4.73 (m, 1H), 4.61-4.53 (m, 1H), 4.48-4.33 (m, 1H), 4.32-4.24 (m, 1H), 4.21-4.14 (m, 2H), 4.11-4.01 (m, 2H), |
| | yl)carbamoyl) | | | 3.95-3.74 (m, 3H), |
| | benzo[b]thio- | | | 3.52 (s, 3H), 2.29- |
| | phen-5- | | | 2.12 (m, 2H), 2.09- |
| | yl)methyl)(phe- | | | 1.97 (m, 2H), 1.95- |
| | noxy)phos- | | | 1.80 (m, 2H), 1.74- |
| | phoryl)-L- | | | 1.63 (m, 1H), 1.60- |
| | alaninate | | | 1.37 (m, 4H), 1.34- |
| | | | | 1.22 (m, 2H), 1.21- |
| | | | | 1.10 (m, 3H), 0.83- |
| | | | | 0.76 (m, 3H), 0.05 (s, |
| | | | | 1H) |
| 329 | ethyl ((fluoro(2- (((3S,6S,7aS, 8aR,9aR)-3- (3-(4- methylpyridin- 3- yl)azetidine- 1-carbonyl)- 5- oxodecahydro- 1H- cyclopropa[d] pyrrolo[1,2- a]azicin-6- yl)carbamoyl) | 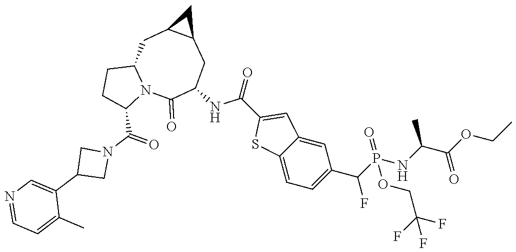 [SMILES: CCOC(=O)[C@H](C)NP(=O)(OCC(F)(F)F)C(F)c1ccc2sc(C(=O)N[C@H]3C[C@@H]4C[C@@H]4C[C@H]4CC[C@@H](C(=O)N5CC(c6cnccc6C)C5)N4C3=O)cc2c1] | 822.7 | ¹H NMR (400 MHz, CDCl₃) δ 9.67-9.49 (m, 1H), 8.89-8.53 (m, 1H), 8.18 (s, 1H), 8.10-8.08 (m, 1H), 7.96-7.77 (m, 2H), 7.63-7.42 (m, 2H), 6.01-5.76 (m, 1H), 5.19 (s, 2H), 4.78- 4.57 (m, 1H), 4.51- 4.45 (m, 1H), 4.42- 4.32 (m, 1H), 4.29- 4.23 (m, 1H), 4.21- 4.07 (m, 3H), 4.06- 3.94 (m, 2H), 3.89- 3.80 (m, 1H), 3.76- |
| | benzo[b]thio- | | | 3.67 (m, 2H), 2.57- |
| | phen-5- | | | 2.42 (m, 4H), 2.34- |
| | yl)meth- | | | 2.31 (m, 2H), 2.24- |
| | yl)(2,2,2- | | | 2.14 (m, 1H), 2.10- |
| | trifluoroethoxy) | | | 1.99 (m, 2H), 1.99- |
| | phosphor- | | | 1.89 (m, 1H), 1.78- |
| | yl)-L- | | | 1.63 (m, 1H), 1.48- |
| | alaninate | | | 1.31 (m, 4H), 1.30- |
| | | | | 1.21 (m, 3H), 0.99- |
| | | | | 0.77 (m, 2H), 0.04- |
| | | | | −0.03 (m, 1H) |
| 325 | propyl ((fluoro(2- (((3S,6S,7aS, 8aR,9aR)-3- (3-(5- methyloxazol- 2- yl)azetidine- 1-carbonyl)- 5- oxodecahydro- 1H- cyclopropa[d] pyrrolo[1,2- a]azocin-6- | 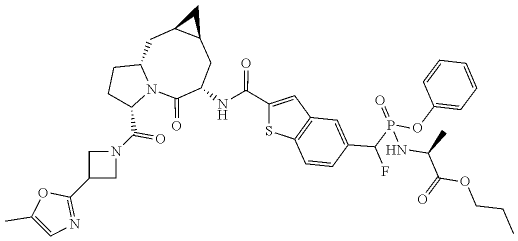 [SMILES: CCCOC(=O)[C@H](C)NP(=O)(Oc1ccccc1)C(F)c1ccc2sc(C(=O)N[C@H]3C[C@@H]4C[C@@H]4C[C@H]4CC[C@@H](C(=O)N5CC(c6ncc(C)o6)C5)N4C3=O)cc2c1] | 820.3 | ¹H NMR (400 MHz, DMSO-d₆) δ 8.90 (m, 1H), 8.32 (m, 1H), 8.07 (m, 2H), 7.62 (m, 1H), 7.41-7.29 (m, 2H), 7.25-7.07 (m, 3H), 6.78 (m, 1H), 6.30-6.19 (m, 1H), 6.17-6.07 (m, 1H), 4.97-4.80 (m, 1H), 4.60-4.48 (m, 1H), 4.42-4.29 (m, 2H), 4.26-4.10 (m, 2H), 4.05-3.98 (m, 1H), 3.96-3.85 (m, |
| | yl)carbamoyl) | | | 4H), 2.39-2.21 (m, |
| | benzo[b]thio- | | | 4H), 2.18-1.93 (m, |
| | phen-5- | | | 4H), 1.90-1.78 (m, |
| | yl)methyl)(phe- | | | 2H), 1.70-1.56 (m, |
| | noxy)phos- | | | 1H), 1.53-1.29 (m, |
| | phoryl)-L- | | | 3H), 1.20-1.09 (m, |
| | alaninate | | | 2H), 0.98-0.90 (m, |
| | | | | 1H), 0.86-0.75 (m, |
| | | | | 4H), 0.71 (s, 1H), |
| | | | | −0.01 (m, 1H) |
| 323 | propyl ((fluoro(2- (((3S,6S,7aS, 8aR,9aR)-3- (3-(oxazol-2- yl)azetidine- 1-carbonyl)- 5- oxodecahydro- 1H- cyclopropa[d] pyrrolo[1,2- a]azocin-6- yl)carbmaoyl) benzo[b]thio- | 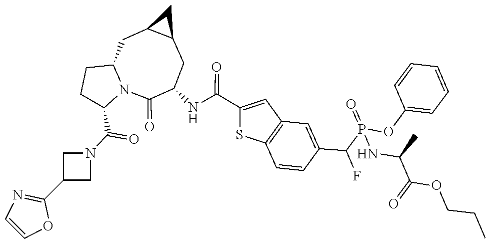 [SMILES: CCCOC(=O)[C@H](C)NP(=O)(Oc1ccccc1)C(F)c1ccc2sc(C(=O)N[C@H]3C[C@@H]4C[C@@H]4C[C@H]4CC[C@@H](C(=O)N5CC(c6ncco6)C5)N4C3=O)cc2c1] | 806.3 | ¹H NMR (400 MHz, CD₃OD) δ 8.13-8.01 (m, 2H), 7.99-7.80 (m, 2H), 7.67-7.56 (m, 1H), 7.36-7.29 (m, 2H), 7.26-7.02 (m, 4H), 6.18-5.97 (m, 1H), 5.05 (m, 1H), 5.00-4.87 (m, 1H), 4.84-4.61 (m, 1H), 4.55-4.37 (m, 2H), 4.33-4.24 (m, 2H), 4.17-4.06 (m, 1H), 4.01-3.74 (m, 3H), 2.50-2.38 (m, |
| | phen-5- | | | 1H), 2.33-2.22 (m, |
| | yl)methyl)(phe- | | | 2H), 2.22-2.08 (m, |
| | noxy)phos- | | | 2H), 2.05-1.89 (m, |
| | phoryl)-L- | | | 2H), 1.78-1.65 (m, |
| | alaninate | | | 1H), 1.60-1.47 (m, |
| | | | | 2H), 1.44-1.35 (m, |
| | | | | 1H), 1.26-1.19 (m, |
| | | | | 2H), 1.06 (d, J = 7.2 |
| | | | | Hz, 1H), 0.94 (m, |
| | | | | 1H), 0.90-0.80 (m, |
| | | | | 4H), 0.10-−0.02 (m, |
| | | | | 1H) |
| 321 | propyl ((fluoro(2- (((3S,6S,7aR, 8aS,9aR)-3- (3-(4- methylpyridin- 3- yl)azetidine- 1-carbonyl)- 5- oxodecahydro- 1H- cyclopropa[d] pyrrolo[1,2- a]azocin-6- | 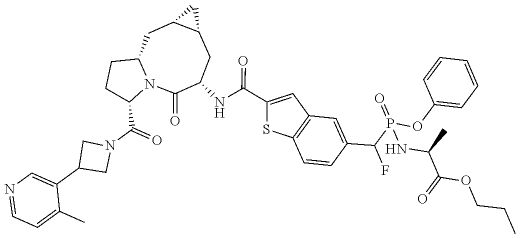 [SMILES: CCCOC(=O)[C@H](C)NP(=O)(Oc1ccccc1)C(F)c1ccc2sc(C(=O)N[C@H]3C[C@H]4C[C@H]4C[C@H]4CC[C@@H](C(=O)N5CC(c6cnccc6C)C5)N4C3=O)cc2c1] | 830.4 | ¹H NMR (400 MHz, CDCl₃) δ 9.29-8.90 (m, 1H), 8.65-8.44 (m, 1H), 8.35-8.14 (m, 1H), 8.09-7.94 (m, 1H), 7.91-7.81 (m, 1H), 7.55 (d, J = 2.9 Hz, 2H), 7.39- 7.32 (m, 1H), 7.26- 7.16 (m, 2H), 7.15- 7.05 (m, 2H), 6.08- 5.90 (m, 1H), 5.14- 4.99 (m, 1H), 4.97- 4.87 (m, 1H), 4.76- 4.66 (m, 1H), 4.48- |
| | yl)carbamoyl) | | | 4.41 (m, 1H), 4.39- |
| | benzo[b]thio- | | | 4.17 (m, 3H), 4.11- |
| | phen-5- | | | 3.92 (m, 2H), |
| | yl)methyl)(phe- | | | 2.52 (d, J = |
| | noxy)phos- | | | 4.3 Hz, 3H), 2.29- |
| | phoryl)-L- | | | 2.22 (m, 1H), 2.16- |
| | alaninate | | | 2.07 (m, 3H), 2.04- |
| | | | | 1.95 (m, 2H), 1.75- |
| | | | | 1.67 (m, 1H), 1.66- |
| | | | | 1.58 (m, 2H), 1.56- |
| | | | | 1.50 (m, 1H), 1.34- |
| | | | | 1.26 (m, 3H), 1.23- |
| | | | | 1.12 (m, 1H), 0.93- |
| | | | | 0.88 (m, 2H), 0.86- |
| | | | | 0.82 (m, 1H), 0.25- |
| | | | | 0.19 (m, 1H) |
| 319 | propyl ((fluoro(2- (((3S,6S,7aR, 8aS,9aR)-3- (3-(4- methoxypyri- din-3- yl)azetidine- 1-carbonyl)- 5- oxodecahydro- 1H- cyclopropa[d] pyrrolo[1,2- a]azocin-6- yl)carbamoyl) benzo[b]thio- phen-5- | 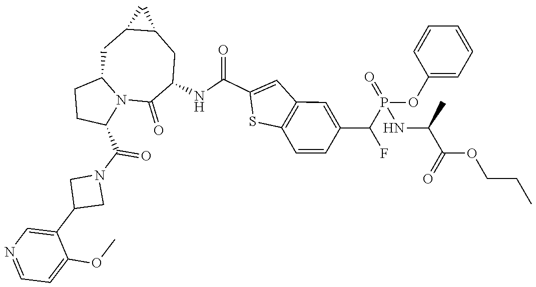 [SMILES: CCCOC(=O)[C@H](C)NP(=O)(Oc1ccccc1)C(F)c1ccc2sc(C(=O)N[C@H]3C[C@H]4C[C@H]4C[C@H]4CC[C@@H](C(=O)N5CC(c6cnccc6OC)C5)N4C3=O)cc2c1] | 846.8 | ¹H NMR (400 MHz, CD₃OD) 8.71-8.57 (m, 2H), 8.15-8.02 (m, 2H), 8.02-7.90 (m, 1H), 7.69-7.49 (m, 2H), 7.39-7.29 (m, 2H), 7.28-7.12 (m, 3H), 6.21-5.97 (m, 1H), 5.01-4.90 (m, 2H), 4.69-4.60 (m, 1H), 4.47-4.30 (m, 3H), 4.25-4.08 (m, 5H), 4.04-3.77 (m, 3H), 2.43-2.32 (m, 1H), 2.32-2.11 (m, 3H), 2.07-1.92 (m, 2H), 1.92-1.78 (m, 1H), 1.77-1.65 |
| | yl)methyl)(phe- | | | (m, 1H), 1.63-1.50 |
| | noxy)phos- | | | (m, 2H), 1.50 (d, J = |
| | phoryl)-L- | | | 2.0 Hz, 1H), 1.39- |
| | alaninate | | | 1.27 (m, 1H), 1.23 |
| | | | | (t, J = 6.8 Hz, 2H), |
| | | | | 1.14-1.02 (m, 1H), |
| | | | | 0.94-0.82 (m, 4H), |
| | | | | 0.19 (d, |
| | | | | J = 4.4 Hz, 1H) |
| 316 | propyl ((fluoro(2- (((3S,6S,7aS, 8aR,9aR)-3- (3-(isoxazol- 3- yl)azetidine- 1-carbonyl)- 5- oxodecahydro- 1H- cyclopropa[d] pyrrolo[1,2- a]azocin-6- yl)carbamoyl) benzo[b]thio- phen-5- yl)methyl)(phe- noxy)phos- | 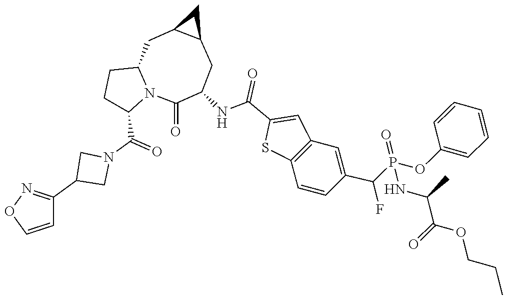 [SMILES: CCCOC(=O)[C@H](C)NP(=O)(Oc1ccccc1)C(F)c1ccc2sc(C(=O)N[C@H]3C[C@@H]4C[C@@H]4C[C@H]4CC[C@@H](C(=O)N5CC(c6ccon6)C5)N4C3=O)cc2c1] | 806.3 | ¹H NMR (400 MHz, CDCl₃) δ 8.44 (s, 1H), 7.99 (d, J = 10.0 Hz, 1H), 7.92-7.84 (m, 1H), 7.81 (s, 1H), 7.63-7.52 (m, 1H), 7.51-7.33 (m, 2H), 7.28 (s, 1H), 7.16- 7.04 (m, 2H), 6.71- 6.34 (m, 1H), 6.10- 5.87 (m, 1H), 5.12- 5.05 (m, 1H), 4.65- 4.53 (m, 1H), 4.50- 4.35 (m, 2H), 4.26- 3.98 (m, 6H), 3.98- 3.73 (m, 1H), 3.73- 3.51 (m, 1H), 2.37- 2.28 (m, 3H), 2.09- 2.05 (m, 3H), 1.75- |
| | phoryl)-L- | | | 1.66 (m, 1H), 1.66- |
| | alaninate | | | 1.57 (m, 2H), 1.46 |
| | | | | (d, J = 5.6 Hz, |
| | | | | 1H), 1.33- |
| | | | | 1.25 (m, 3H), 1.13 |
| | | | | (d, J = 7.2 Hz, 1H), |
| | | | | 1.04-0.96 (m, 1H), |
| | | | | 0.93 (t, J = 7.2 Hz, |
| | | | | 2H), 0.90-0.81 (m, |
| | | | | 2H), 0.03 (d, J = 5.2 |
| | | | | Hz, 1H) |
| 315 | propyl ((fluoro(2- (((3S,6S,7aS, 8aR,9aR)-5- oxo-3-(3- (pyrimidin-2- yl)azetidine- 1- carbonyl)deca- hydro-1H- cyclopropa[d] pyrrolo[1,2- a]azocin-6- yl)carbamoyl) benzo[b]thio- phen-5- | 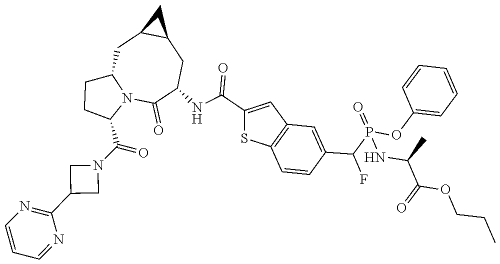 [SMILES: CCCOC(=O)[C@H](C)NP(=O)(Oc1ccccc1)C(F)c1ccc2sc(C(=O)N[C@H]3C[C@@H]4C[C@@H]4C[C@H]4CC[C@@H](C(=O)N5CC(c6ncccn6)C5)N4C3=O)cc2c1] | 817.2 | ¹H NMR (400 MHz, CDCl₃) δ 8.75 (d, J = 4.8 Hz, 2H), 8.00- 7.89 (m, 1H), 7.89- 7.73 (m, 2H), 7.63- 7.41 (m, 2H), 7.38- 7.31 (m, 1H), 7.25- 7.17 (m, 2H), 7.16- 7.04 (m, 2H), 6.06- 5.86 (m, 1H), 5.12- 4.90 (m, 2H), 4.62- 4.52 (m, 2H), 4.52- 4.35 (m, 1H), 4.28- 4.11 (m, 3H), 4.10- 3.94 (m, 2H), 3.88- 3.55 (m, 1H), 2.39- |
| | yl)methyl)(phe- | | | 2.24 (m, 3H), 2.18- |
| | noxy)phos- | | | 1.99 (m, 4H), 1.67 (s, |
| | phoryl)-L- | | | 3H), 1.54-1.46 (m, |
| | alaninate | | | 1H), 1.32-1.08 (m, |
| | | | | 3H), 1.06-0.97 (m, |
| | | | | 1H), 0.95-0.79 (m, |
| | | | | 4H), 0.04-−0.05 (m, |
| | | | | 1H) |
| 314 | S,S′- (((difluoro(2- (((3S,6S,7aS, 8aR,9aR)-5- oxo-3-(3- (pyridin-2- yl)azetidine- 1- carbonyl)deca- hydro-1H- cyclopropa[d] pyrrolo[1,2- a]azocin-6- yl)carbamoyl) benzo[b]thio- phen-5- yl)methyl)phos- phoryl)bis (oxy))bis(eth ane-2,1- | 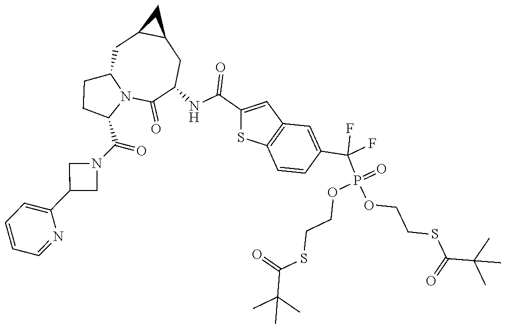 [SMILES: CC(C)(C)C(=O)SCCOP(=O)(OCCSC(=O)C(C)(C)C)C(F)(F)c1ccc2sc(C(=O)N[C@H]3C[C@@H]4C[C@@H]4C[C@H]4CC[C@@H](C(=O)N5CC(c6ccccn6)C5)N4C3=O)cc2c1] | 933.3 | ¹H NMR (400 MHz, CDCl₃) δ 8.70-8.57 (m, 1H), 8.12 (d, J = 5.6 Hz, 1H), 7.98- 7.92 (m, 1H), 7.91- 7.85 (m, 1H), 7.75- 7.59 (m, 2H), 7.58- 7.49 (m, 1H), 7.44- 7.33 (m, 1H), 7.26- 7.18 (m, 1H), 5.10- 4.78 (m, 2H), 4.67- 4.36 (m, 3H), 4.32- 4.03 (m, 7H), 3.18- 2.97 (m, 4H), 2.37- 2.27 (m, 3H), 2.15- 2.06 (m, 4H), 1.74- 1.64 (m, 1H), 1.54- 1.44 (m, 1H), 1.18 (d, J = 1.2 Hz, 18H), 1.05-0.95 (m, 1H), |
| | diyl))bis(2,2- | | | 0.83 (t, J = 4.8 Hz, |
| | dimethyl- | | | 1H), 0.01 (d, J = |
| | propane- | | | 5.2 Hz, 1H) |
| | thioate) | | | |
| 311 | propyl(((2- (((3S,6S,7aS, 8aR,9aR)-3- (3-(4- methylpyridin- 3- yl)azetidine- 1-carbonyl)- 5- oxodecahydro- 1H- cyclopropa[d] pyrrolo[1,2- a]azocin-6- yl)carbamoyl) benzo[b]thio- phen-5- | 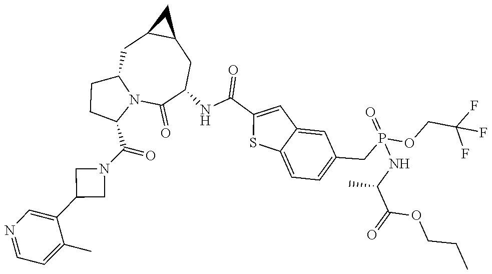 [SMILES: CCCOC(=O)[C@H](C)NP(=O)(Cc1ccc2sc(C(=O)N[C@H]3C[C@@H]4C[C@@H]4C[C@H]4CC[C@@H](C(=O)N5CC(c6cnccc6C)C5)N4C3=O)cc2c1)OCC(F)(F)F] | 818.3 | ¹H NMR (400 MHz, CD₃OD) δ 8.92- 8.81 (m, 1H), 8.66- 8.56 (m, 1H), 8.13- 8.00 (m, 1H), 7.94- 7.81 (m, 3H), 7.48- 7.38 (m, 1H), 5.12- 4.96 (m, 2H), 4.79- 4.72 (m, 1H), 4.58- 4.49 (m, 2H), 4.48- 4.41 (m, 1H), 4.39- 4.27 (m, 3H), 4.14 (dd, J = 6.4, 9.6 Hz, 1H), 4.10-3.96 (m, 2H), 3.95-3.77 (m, 1H), 3.51-3.39 (m, 2H), 2.60-2.53 (m, |
| | yl)methyl) | | | 3H), 2.52-2.40 (m, |
| | (2,2,2- | | | 1H), 2.36-2.10 (m, |
| | trifluoroeth- | | | 4H), 2.07-1.92 (m, |
| | oxy)phosphor- | | | 2H), 1.75 (dt, J = |
| | yl)-L- | | | 7.2, 13.6 Hz, 1H), |
| | alaninate | | | 1.62 (qd, J = 7.2, |
| | | | | 18.4 Hz, 2H), 1.46- |
| | | | | 1.29 (m, 3H), 1.21 |
| | | | | (d, J = 7.2 Hz, 1H), |
| | | | | 1.01-0.81 (m, 5H), |
| | | | | 0.12-0.04 (m, 1H) |
| 310 | propyl(((2- (((3S,6S,7aS, 8aR,9aR)-3- (3-(1,1-di- oxidothiomor- pholino)aze- tidine-1- carbonyl)-5- oxodecahydro- 1H- cyclopropa[d] pyrrolo[1,2- a]azocin-6- yl)carbamoyl) benzo[b]thio- phen-5- | 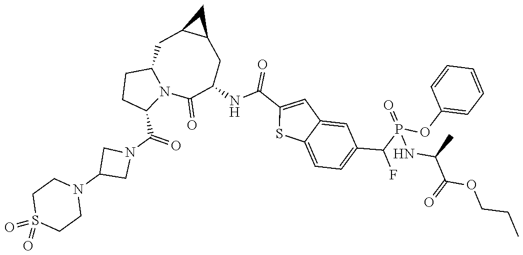 [SMILES: CCCOC(=O)[C@H](C)NP(=O)(Oc1ccccc1)C(F)c1ccc2sc(C(=O)N[C@H]3C[C@@H]4C[C@@H]4C[C@H]4CC[C@@H](C(=O)N5CC(N6CCS(=O)(=O)CC6)C5)N4C3=O)cc2c1] | 872.2 | ¹H NMR (400 MHz, CDCl₃) δ 7.96 (s, 1H), 7.93-7.76 (m, 2H), 7.63-7.52 (m, 1H), 7.50-7.32 (m, 2H), 7.32-7.28 (m, 1H), 7.25 (d, J = 1.6 Hz, 1H), 7.15-7.05 (m, 1H), 6.12-5.84 (m, 1H), 5.16-5.04 (m, 1H), 4.82-4.48 (m, 1H), 4.44-4.36 (m, 1H), 4.31-4.19 (m, 2H), 4.18-4.10 (m, 1H), 4.08- 4.05 (m, 1H), 4.03- |
| | yl)fluorometh- | | | 3.98 (m, 1H), 3.94- |
| | yl)(phenoxy) | | | 3.88 (m, 1H), 3.81- |
| | phosphoryl)- | | | 3.72 (m, 1H), 3.71- |
| | L-alaninate | | | 3.62 (m, 1H), 3.60- |
| | | | | 3.46 (m, 2H), 3.24- |
| | | | | 3.10 (m, 4H), 3.04- |
| | | | | 2.93 (m, 3H), 2.33 |
| | | | | (d, J = 14.4 Hz, |
| | | | | 3H), 2.21- |
| | | | | 2.11 (m, 1H), 2.11- |
| | | | | 1.98 (m, 3H), 1.74- |
| | | | | 1.49 (m, 3H), 1.46- |
| | | | | 1.35 (m, 1H), 1.33- |
| | | | | 1.11 (m, 3H), 0.98- |
| | | | | 0.89 (m, 4H), 0.88- |
| | | | | 0.84 (m, 1H), 0.11- |
| | | | | −0.03 (m, 1H) |
| 309 | propyl ((fluoro(2- (((3S,6S,7aS, 8aR,9aR)-3- (3-(4-methyl- 4H-1,2,4- triazol-3- yl)azetidine- 1-carbonyl)- 5- oxodecahydro- 1H- cyclopropa[d] pyrrolo[1,2- a]azocin-6- yl)carbamoyl) | 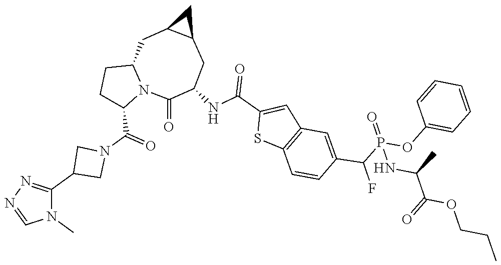 [SMILES: CCCOC(=O)[C@H](C)NP(=O)(Oc1ccccc1)C(F)c1ccc2sc(C(=O)N[C@H]3C[C@@H]4C[C@@H]4C[C@H]4CC[C@@H](C(=O)N5CC(c6nncn6C)C5)N4C3=O)cc2c1] | 820.5 | ¹H NMR (400 MHz, CDCl₃) δ 9.12-8.70 (m, 1H), 8.01-7.93 (m, 1H), 7.91-7.74 (m, 2H), 7.63-7.51 (m, 1H), 7.35 (d, J = 7.2 Hz, 2H), 7.25- 7.13 (m, 2H), 7.11- 6.99 (m, 1H), 6.09- 5.88 (m, 1H), 5.19- 4.96 (m, 2H), 4.76- 4.56 (m, 1H), 4.47- 4.36 (m, 1H), 4.34- 4.17 (m, 2H), 3.92 (s, 4H), 3.88-3.61 (m, 4H), 2.47-2.25 (m, |
| | benzo[b]thio- | | | 3H), 2.21-2.12 (m, |
| | phen-5- | | | 1H), 2.10-2.00 (m, |
| | yl)methyl)(phe- | | | 2H), 1.73-1.49 (m, |
| | noxy)phos- | | | 3H), 1.47-1.36 (m, |
| | phoryl)-L- | | | 1H), 1.35-1.24 (m, |
| | alaninate | | | 3H), 1.22-1.12 (m, |
| | | | | 1H), 0.99-0.79 (m, |
| | | | | 5H), 0.09-0.04 (m, |
| | | | | 1H) |
| 308 | S,S′- ((((difluoro(2- (((3S,6S,7aS, 8aR,9aR)-3- (3-(4- fluoropyridin- 3- yl)azetidine- 1-carbonyl)- 5- oxodecahydro- 1H- cyclopropa[d] pyrrolo[1,2- a]azocin-6- yl)carbamoyl) benzo[b]thio- phen-5- yl)methyl)phos- phoryl)bis | 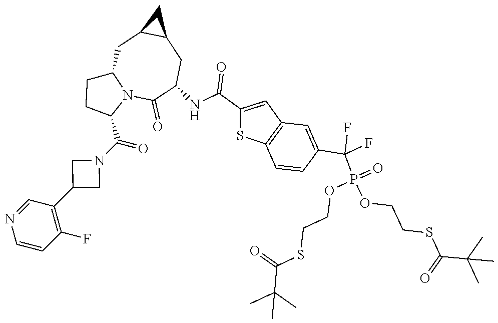 [SMILES: CC(C)(C)C(=O)SCCOP(=O)(OCCSC(=O)C(C)(C)C)C(F)(F)c1ccc2sc(C(=O)N[C@H]3C[C@@H]4C[C@@H]4C[C@H]4CC[C@@H](C(=O)N5CC(c6cnccc6F)C5)N4C3=O)cc2c1] | 951.2 | ¹H NMR (400 MHz, DMSO-d₆) δ 9.08- 8.91 (m, 1H), 8.67 (d, J = 10.4 Hz, 1H), 8.52 (m, 1H), 8.36 (d, J = 12.0 Hz, 1H), 8.26- 8.20 (m, 2H), 7.65- 7.622 (m, 1H), 7.32 (m, 1H), 5.01-4.88 (m, 1H), 4.63 (d, J = 7.2 Hz, 1H), 4.41- 4.35 (m, 1H), 4.32- 3.89 (m, 9H), 3.11- 3.06 (m, 4H), 2.19- 1.96 (m, 4H), 1.91- 1.77 (m, 2H), 1.70- 1.57 (m, 1H), 1.44- 1.29 (m, 1H), 1.20- 1.16 (m, 1H), 1.14- |
| | (oxy))bis | | | 1.07 (m, 18H), 0.88- |
| | (ethane- | | | 0.76 (m, 1H), 0.741- |
| | 2,1-diyl)) | | | 0.691 (m, 1H), 0.00 |
| | bis(2,2- | | | (d, J = 4.4 Hz, 1H) |
| | dimethyl- | | | |
| | propane- | | | |
| | thioate) | | | |
| 306 | S,S′- ((((fluoro(2- (((3S,6S,7aS, 8aR,9aR)-3- (3-(4- fluoropyridin- 3- yl)azetidine- 1-carbonyl)- 5- oxodecahydro- 1H- cyclopropa[d] pyrrolo[1,2- a]azocin-6- | 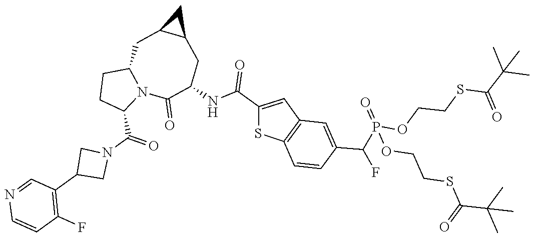 [SMILES: CC(C)(C)C(=O)SCCOP(=O)(OCCSC(=O)C(C)(C)C)C(F)c1ccc2sc(C(=O)N[C@H]3C[C@@H]4C[C@@H]4C[C@H]4CC[C@@H](C(=O)N5CC(c6cnccc6F)C5)N4C3=O)cc2c1] | 933.5 | ¹H NMR (400 MHz, CDCl₃) δ 8.90-8.52 (m, 2H), 8.01 (s, 1H), 7.95- 7.82 (m, 2H), 7.63-7.37 (m, 2H), 7.07 (m, 1H), 6.12- 5.91 (m, 1H), 5.14- 4.86 (m, 2H), 4.69- 4.56 (m, 1H), 4.51- 4.33 (m, 2H), 4.31- 4.07 (m, 7H), 3.23- 3.07 (m, 2H), 2.97- 2.75 (m, 2H), 2.41- 2.27 (m, 3H), 2.19- |
| | yl)carbamoyl) | | | 2.03 (m, 4H), 1.52- |
| | benzo[b]thio- | | | 1.43 (m, 1H), 1.28- |
| | phen-5-yl) | | | 1.12 (m, 19H), 1.05- |
| | methyl)phos- | | | 0.96 (m, 1H, 0.89- |
| | phoryl) | | | 0.80 (m, 1H), 0.02 |
| | bis(oxy)) | | | (m, 1H) |
| | bis(ethane- | | | |
| | 2,1-diyl)) | | | |
| | bis(2,2-di- | | | |
| | methylpropane- | | | |
| | thioate) | | | |
| 287 | propyl ((fluoro(2- (((3S,6S,7aS, 8aR,9aR)-3- (3-(4- methylpyridin- 3- yl)azetidine- 1-carbonyl)- 5- oxodecahydro- 1H- cyclopropa[d] pyrrolo[1,2- a]azocin-6- yl)carbamoyl) benzo[b]thio- | 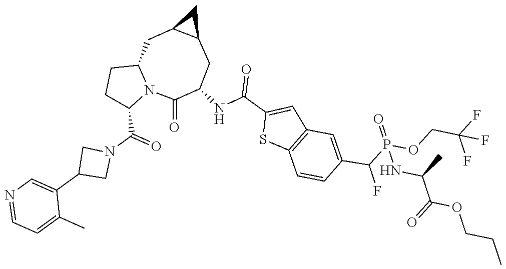 [SMILES: CCCOC(=O)[C@H](C)NP(=O)(OCC(F)(F)F)C(F)c1ccc2sc(C(=O)N[C@H]3C[C@@H]4C[C@@H]4C[C@H]4CC[C@@H](C(=O)N5CC(c6cnccc6C)C5)N4C3=O)cc2c1] | 836.6 | ¹H NMR (400 MHz, CDCl₃) δ 9.78-9.51 (m, 1H), 8.75-8.57 (m, 1H), 8.24-8.10 (m, 1H), 7.95-7.89 (m, 1H), 7.86-7.81 (m, 1H), 7.58 (d, J = 5.6 Hz, 1H), 7.53- 7.43 (m, 1H), 5.99- 5.78 (m, 1H), 5.30- 5.06 (m, 2H), 4.76- 4.63 (m, 1H), 4.52- 4.44 (m, 2H), 4.41- 4.20 (m, 3H), 4.15- 3.90 (m, 5H), 2.51 (s, 3H), 2.48 (s, 1H), 2.34 (d, J = 3.6 Hz, |
| | phen-5- | | | 1H), 2.31 (s, 1H), |
| | yl)methyl) | | | 2.22-2.13 (m, 1H), |
| | (2,2,2-tri- | | | 2.10-1.99 (m, 2H), |
| | fluoroethoxy)- | | | 1.93 (d, J = 7.6 Hz, |
| | phosphor- | | | 1H), 1.77-1.55 (m, |
| | yl)-L- | | | 3H), 1.46-1.31 (m, |
| | alaninate | | | 3H), 1.30-1.21 (m, |
| | | | | 1H), 1.01-0.88 (m, |
| | | | | 3H), 0.87-0.77 (m, |
| | | | | 2H), 0.08-0.04 (m, |
| | | | | 1H) |
| 269 | propyl(((2- (((3S,6S,7aR, 8aS,9aR)-3- (((3S,4S)-4- fluoro-1- (pyridin-3- yl)pyrrolidin- 3- yl)carbamoyl)- 5- oxodecahydro- 1H- cyclopropa[d] pyrrolo[1,2- a]azocin-6- yl)carbamoyl) benzo[b]thio- | 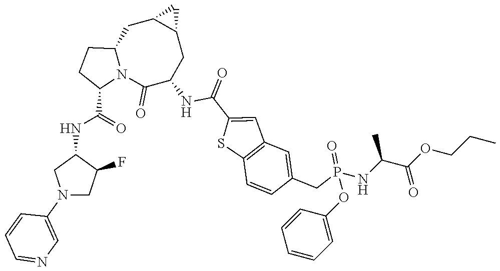 [SMILES: CCCOC(=O)[C@H](C)NP(=O)(Cc1ccc2sc(C(=O)N[C@H]3C[C@H]4C[C@H]4C[C@H]4CC[C@@H](C(=O)N[C@H]5CN(c6cccnc6)C[C@@H]5F)N4C3=O)cc2c1)Oc1ccccc1] | 845.4 | ¹H NMR (400 MHz, DMSO-d₆) δ 8.72 (d, J = 7.2 Hz, 1H), 8.43 (d, J = 6.8 Hz, 1H), 8.19-8.13 (m, 2H), 8.11 (d, J = 4.8 Hz, 1H), 7.97-7.85 (m, 2H), 7.78-7.60 (m, 2H), 7.44 (d, J = 8.4 Hz, 1H), 7.38-7.29 (m, 2H), 7.19-7.08 (m, 3H), 5.73-5.55 (m, 1H), 5.27-5.09 (m, 1H), 4.82-4.77 (m, 1H), 4.49-4.40 (m, 1H), 4.22-4.15 (m, 2H), 3.90-3.85 |
| | phen-5- | | | (m, 2H), 3.75 (s, |
| | yl)methyl)(phe- | | | 2H), 3.71 (s, |
| | noxy)phos- | | | 1H), 3.64 (s, |
| | phoryl)-L- | | | 1H), 3.40 (s, 1H), |
| | alaniante | | | 3.37 (s, 1H), 2.28- |
| | | | | 2.24 (m, 1H), 2.15- |
| | | | | 1.97 (m, 4H), 1.93- |
| | | | | 1.81 (m, 2H), 1.70- |
| | | | | 1.66 (m, 1H), 1.57 |
| | | | | (d, J = 10.0 Hz, |
| | | | | 1H), 1.51-1.44 |
| | | | | (m, 2H), 1.37-1.28 |
| | | | | (m, 1H), 1.23-1.20 |
| | | | | (m, 1H), 1.07 (d, |
| | | | | J = 7.2 Hz, 1H), 1.01 |
| | | | | (d, J = 7.2 Hz, 2H), |
| | | | | 0.85-0.76 (m, 4H), |
| | | | | 0.08-0.05 (m, 1H) |
| 268 | propyl ((fluoro(2- (((3S,6S,7aR, 8aS,9aR)-3- (((3S,4S)-4- fluoro-1- (pyridin-3- yl)pyrrolidin- 3- yl)carbamoyl)- 5- oxodecahydro- 1H- cyclopropa[d] pyrrolo[1,2- a]azocin-6- yl)carbamoyl) benzo[b]thio- phen-5- yl)methyl)(phe- noxy)phos- phoryl)-L- alaninate | 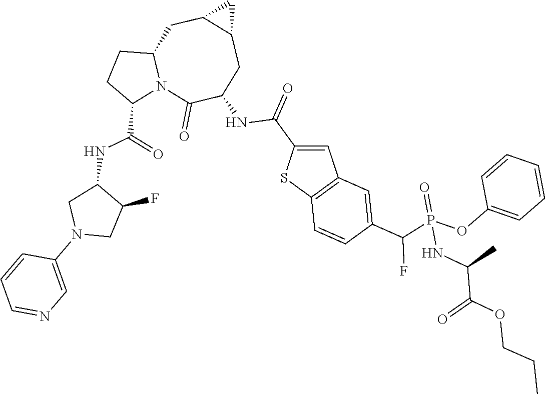 [SMILES: CCCOC(=O)[C@H](C)NP(=O)(Oc1ccccc1)C(F)c1ccc2sc(C(=O)N[C@H]3C[C@H]4C[C@H]4C[C@H]4CC[C@@H](C(=O)N[C@H]5CN(c6cccnc6)C[C@@H]5F)N4C3=O)cc2c1] | 863.6 | ¹H NMR (400 MHz, DMSO-d₆) δ 8.86- 8.73 (m, 1H), 8.43 (d, J = 6.8 Hz, 1H), 8.26 (s, 1H), 8.19-8.02 (m, 4H), 7.73-7.53 (m, 3H), 7.44-7.30 (m, 2H), 7.26-7.10 (m, 3H), 6.31-6.08 (m, 2H), 5.27-5.09 (m, 1H), 4.82-4.79 (m, 1H), 4.47-4.41 (m, 1H), 4.26-4.13 (m, 2H), 3.97-3.83 (m, 3H), 3.78-3.69 (m, 3H), 3.63 (s, 1H), 2.31- 2.22 (m, 1H), 2.13-1.98 (m, 3H), 1.94-1.80 (m, 2H), 1.58 (d, J = 10.4 Hz, 2H), 1.53-1.46 (m, 2H), 1.38-1.28 (m, |
| | | | | 1H), 1.27-1.11 (m, |
| | | | | 4H), 0.98-0.94 (m, |
| | | | | 1H), 0.88-0.74 (m, |
| | | | | 4H) |
| 264 | propyl (((2- (((3S,6S,7aS, 8aR,9aR)-3- (3-(4- fluoropyridin- 3- yl)azetidine- 1-carbonyl)- 5- oxodecahydro- 1H- cyclopropa[d] pyrrolo[1,2- a]azocin-6- yl)carbamoyl) | 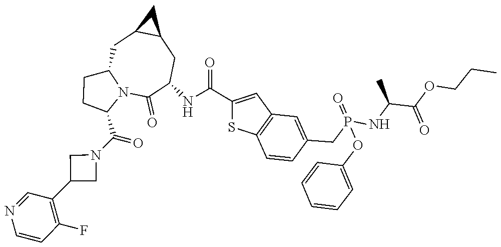 [SMILES: CCCOC(=O)[C@H](C)NP(=O)(Cc1ccc2sc(C(=O)N[C@H]3C[C@@H]4C[C@@H]4C[C@H]4CC[C@@H](C(=O)N5CC(c6cnccc6F)C5)N4C3=O)cc2c1)Oc1ccccc1] | 816.3 | ¹H NMR (400 MHz, CDCl₃) δ 8.59-8.74 (m, 1H) 7.99-8.31 (m, 2H) 7.75 (m, 3H) 7.26 (s, 5H) 7.12 (m, 3H) 4.96-5.19 (m, 2H) 4.54-4.73 (m, 1H) 4.44 (m, 2H), 4.05-4.28 (m, 3H) 3.86-4.02 (m, 3H) 3.36-3.51 (m, 2H) 2.25-2.51 (m, 3H) 1.95-2.20 (m, 4H) 1.35-1.73 (m, 5H) 1.11-1.20 (m, 3H) |
| | benzo[b]thio- | | | 0.80-0.91 (m, 4H) |
| | phen-5- | | | −0.09-0.08 (m, 1H) |
| | yl)methyl)(phe- | | | |
| | noxy)phos- | | | |
| | phoryl)-L- | | | |
| | alaninate | | | |
| 263 | propyl ((fluoro(2- (((3S,6S,7aS, 8aR,9aR)-3- (3-(4- fluoropyridin- 3- yl)azetidine- 1-carbonyl)- 5- oxodecahydro- 1H- cyclopropa[d] pyrrolo[1,2- a]azocin-6- yl)carbamoyl) benzo[b]thio- | 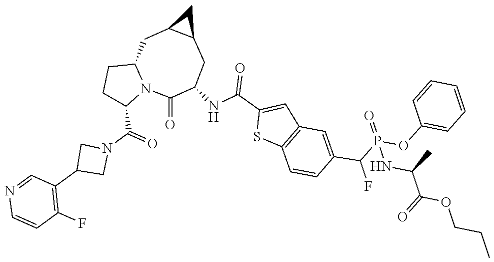 [SMILES: CCCOC(=O)[C@H](C)NP(=O)(Oc1ccccc1)C(F)c1ccc2sc(C(=O)N[C@H]3C[C@@H]4C[C@@H]4C[C@H]4CC[C@@H](C(=O)N5CC(c6cnccc6F)C5)N4C3=O)cc2c1] | 834.3 | ¹H NM (400 MHz, CDCl₃) δ = 9.57 (dd, J = 8.4, 14.0 Hz, 1H), 9.47 (d, J = 7.6 Hz, 1H), 8.86-8.60 (m, 1H), 8.04-7.72 (m, 4H), 7.64-7.50 (m, 1H), 7.44-7.27 (m, 3H), 7.25-7.04 (m, 3H), 6.15-5.77 (m, 1H), 5.29-4.96 (m, 2H), 4.73 (s, 1H), 4.50-4.40 (m, 2H), 4.15-4.09 (m, 2H), 4.09-4.01 (m, 2H), 4.00-3.78 (m, 1H), 3.76-3.54 (m, 1H), |
| | phen-5- | | | 2.51-2.27 (m, 3H), |
| | yl)methyl)(phe- | | | 2.17 (td, J = 5.6, 11.6 |
| | noxy)phos- | | | Hz, 1H), 2.10-1.91 |
| | phoryl)-L- | | | (m, 3H), 1.78-1.50 |
| | alaniante | | | (m, 3H), 1.40-1.25 |
| | | | | (m, 3H), 1.18-0.76 |
| | | | | (m, 6H), 0.09-−0.06 |
| | | | | (m, 1H) |
| 255 | propyl(((2- (((3S,6S,7aR, 8aS,9aR)-3- (((3S,4S)-1- acetyl-4- fluoropyrroli- din-3- yl)carbamoyl)- 5- oxodecahydro- 1H- cyclopropa[d] pyrrolo[1,2- a]azocin-6- yl)carbamoyl) benzo[b]thio- phen-5- yl)fluorometh- yl)(phenoxy) phosphoryl)- L-alaninate | 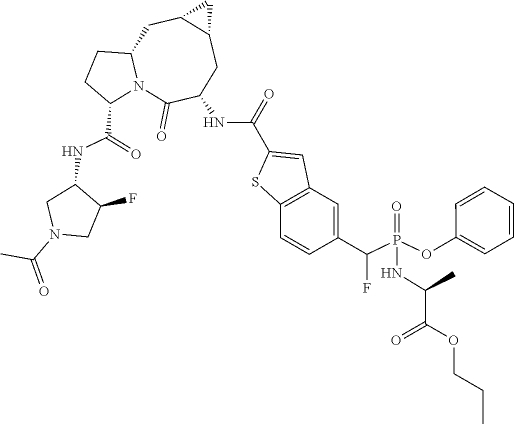 [SMILES: CCCOC(=O)[C@H](C)NP(=O)(Oc1ccccc1)C(F)c1ccc2sc(C(=O)N[C@H]3C[C@H]4C[C@H]4C[C@H]4CC[C@@H](C(=O)N[C@H]5CN(C(C)=O)C[C@@H]5F)N4C3=O)cc2c1] | 828.4 | ¹H NMR (400 MHz, CDCl₃) δ 8.30-8.18 (m, 1H), 8.08-7.85 (m, 3H), 7.68-7.51 (m, 1H), 7.41-7.32 (m, 1H), 7.29 (d, J = 3.2 Hz, 1H), 7.25- 7.18 (m, 1H), 7.14- 7.06 (m, 1H), 6.10 (s, 1H), 5.04-4.91 (m, 1H), 4.73-4.41 (m, 2H), 4.25 (t, J = 5.6 Hz, 1H), 4.13-3.98 (m, 3H), 3.82-3.69 (m, 2H), 3.67-3.48 (m, 1H), 3.46-3.24 (m, 2H), 3.02-2.90 (m, 3H), 2.44-2.31 (m, 1H), 2.23-2.08 (m, 3H), 2.05 (s, 3H), 2.00-1.83 (m, 2H), 1.82-1.70 (m, 1H), 1.68-1.51 (m, 3H), 1.48-1.39 (m, 1H), 1.30 (d, J = 3.2 Hz, 2H), 1.26-1.18 (m, |
| | | | | 1H), 1.16 (d, J = 7.2 |
| | | | | Hz, 1H), 0.96-0.81 |
| | | | | (m, 4H), 0.30-0.14 |
| | | | | (m, 1H) |
| 251 | propyl(((2- (((3S,6S,7aR, 8aS,9aR)-3- (3-(4- fluoropyridin- 3- yl)azetidine- 1-carbonyl)- 5- oxodecahydro- 1H- cyclopropa[d] pyrrolo[1,2- a]azocin-6- yl)carbamoyl) benzo[b]thio- | 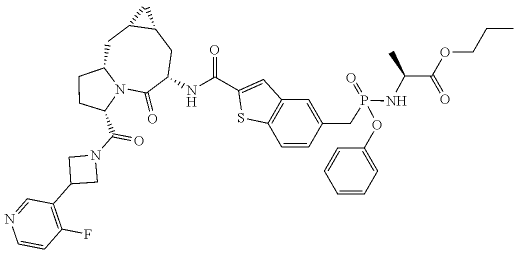 [SMILES: CCCOC(=O)[C@H](C)NP(=O)(Cc1ccc2sc(C(=O)N[C@H]3C[C@H]4C[C@H]4C[C@H]4CC[C@@H](C(=O)N5CC(c6cnccc6F)C5)N4C3=O)cc2c1)Oc1ccccc1] | 816.6 | ¹H NMR (400 MHz, CDCl₃) δ 8.79-8.52 (m, 2H), 7.89-7.72 (m, 4H), 7.51-7.39 (m, 1H), 7.33-7.27 (m, 2H), 7.21-7.00 (m, 4H), 5.01-4.70 (m, 2H), 4.66-4.58 (m, 1H), 4.47-4.34 (m, 2H), 4.33-4.20 (m, 1H), 4.20-4.10 (m, 2H), 4.07-3.87 (m, 3H), 3.52-3.38 (m, 2H), 3.33-3.05 (m, 1H), 2.38-2.29 (m, 1H), 2.25-2.17 |
| | phen-5- | | | (m, 1H), 2.16-2.05 |
| | yl)methyl)(phe- | | | (m, 3H), 2.04-1.93 |
| | noxy)phos- | | | (m, 2H), 1.86-1.69 |
| | phoryl)-L- | | | (m, 2H), 1.63-1.50 |
| | alaninate | | | (m, 2H), 1.28-1.20 |
| | | | | (m, 1H), 1.19-1.11 |
| | | | | (m, 3H), 0.93-0.78 |
| | | | | (m, 4H), 0.25-0.18 |
| | | | | (m, 1H) |
| 249 | propyl ((fluoro(2- (((3S,6S,7aR, 8aS,9aR)-3- (3-(4- fluoropyridin- 3- yl)azetidine- 1-carbonyl)- 5- oxodecahydro- 1H- cyclopropa[d] pyrrolo[1,2- a]azocin-6- yl)carbamoyl) benzo[b]thio- phen-5- | 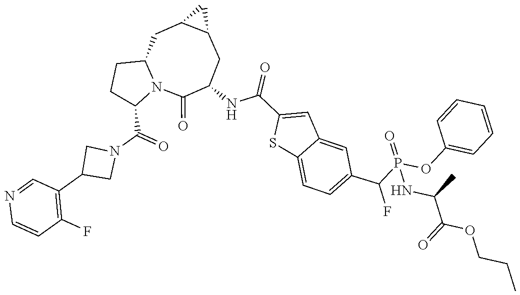 [SMILES: CCCOC(=O)[C@H](C)NP(=O)(Oc1ccccc1)C(F)c1ccc2sc(C(=O)N[C@H]3C[C@H]4C[C@H]4C[C@H]4CC[C@@H](C(=O)N5CC(c6cnccc6F)C5)N4C3=O)cc2c1] | 834.3 | ¹H NMR (400 MHz, CDCl₃) δ 8.61 (d, J = 10.0 Hz, 1H), 8.56 (dd, J = 5.2, 7.6 Hz, 1H), 7.98 (d, J = 10.0 Hz, 1H), 7.91-7.73 (m, 3H), 7.64-7.53 (m, 1H), 7.39-7.32 (m, 1H), 7.26-7.23 (m, 1H), 7.22-7.11 (m, 1H), 7.10-7.03 (m, 2H), 6.11-5.85 (m, 1H), 5.01-4.70 (m, 2H), 4.68-4.57 (m, 1H), 4.45-4.39 (m, 1H), 4.39-4.31 (m, 1H), 4.24-4.11 (m, 3H), 4.09-3.95 |
| | yl)methyl)(phe- | | | (m, 2H), 3.67-3.50 |
| | noxy)phos- | | | (m, 1H), 2.38-2.28 |
| | phoryl)-L- | | | (m, 1H), 2.25-2.17 |
| | alaninate | | | (m, 1H), 2.16-2.04 |
| | | | | (m, 3H), 2.02-1.93 |
| | | | | (m, 2H), 1.85-1.75 |
| | | | | (m, 1H), 1.74-1.69 |
| | | | | (m, 1H), 1.61-1.50 |
| | | | | (m, 2H), 1.32-1.23 |
| | | | | (m, 3H), 1.21-1.09 |
| | | | | (m, 1H), 0.94-0.89 |
| | | | | (m, 2H), 0.89-0.78 |
| | | | | (m, 2H), 0.22 (q, J = |
| | | | | 4.4 Hz, 1H) |
| 245 | propyl(((2- (((3S,6S,7aR, 8aS,9aR)-3- (((3S,4S)-1- acetyl-4- fluoropyrroli- din-3- yl)carbamoyl)- 5- oxodecahydro- 1H- cyclopropa[d] pyrrolo[1,2- a]azocin-6- yl)carbamoyl) benzo[b]thio- | 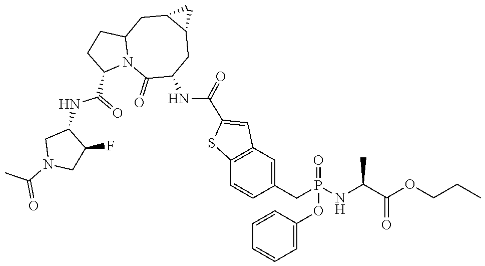 [SMILES: CCCOC(=O)[C@H](C)NP(=O)(Cc1ccc2sc(C(=O)N[C@H]3C[C@H]4C[C@H]4CC4CC[C@@H](C(=O)N[C@H]5CN(C(C)=O)C[C@@H]5F)N4C3=O)cc2c1)Oc1ccccc1] | 810.2 | ¹H NMR (400 MHz, CDCl₃) δ 8.25-8.15 (m, 1H), 7.98-7.90 (m, 1H), 7.90-7.81 (m, 2H), 7.50-7.41 (m, 1H), 7.33-7.27 (m, 2H), 7.18-7.09 (m, 3H), 5.03-4.90 (m, 1H), 4.75-4.45 (m, 2H), 4.32-4.18 (m, 1H), 4.07 (t, J = 8.0 Hz, 1H), 4.03- 3.90 (m, 3H), 3.77 (dd, J = 4.8, 10.8 Hz, 2H), 3.48 (d, J = 5.2 Hz, 2H), 3.35-3.21 |
| | phen-5- | | | (m, 2H), 2.45-2.31 |
| | yl)methyl)(phe- | | | (m, 1H), 2.22-2.15 |
| | noxy)phos- | | | (m, 1H), 2.12 (d, J = |
| | phoryl)-L- | | | 7.2 Hz, 1H), 2.08 (s, |
| | alaninate | | | 1H), 2.04 (s, 3H), |
| | | | | 2.00-1.85 (m, 2H), |
| | | | | 1.82-1.69 (m, 1H), |
| | | | | 1.65-1.53 (m, 3H), |
| | | | | 1.47-1.36 (m, 1H), |
| | | | | 1.25 (d, J = 15.6 Hz, |
| | | | | 1H), 1.19 (d, J = 7.2 |
| | | | | Hz, 2H), 1.15 (d, J = |
| | | | | 7.2 Hz, 1H), 0.92- |
| | | | | 0.82 (m, 4H), 0.25- |
| | | | | 0.17 (m, 1H) |
| 426 | propyl(((S)- (2- (((3S,6S,7aS, 8aR,9aR)-3- (3-(4- (azetidin-1- yl)pyridin-3- yl)azetidine- 1-carbonyl)- 5- oxodecahydro- 1H- cyclopropa[d] pyrrolo[1,2- a]azocin-6- yl)carbamoyl) benzo[b]thio- phen-5- yl)fluorometh- | 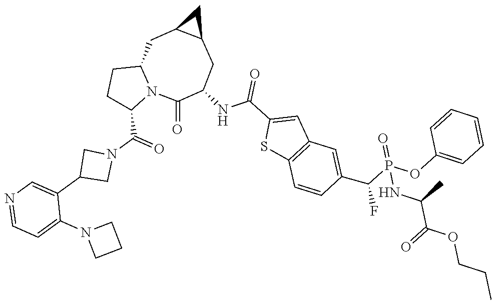 | 871.4 | 1H NMR (400 MHz, CD₃OD) δ 8.27- 8.18 (m, 1H), 8.13- 7.84 (m, 4H), 7.68- 7.58 (m, 1H), 7.39- 7.30 (m, 2H), 7.27- 7.15 (m, 3H), 6.48- 6.25 (m, 1H), 6.19- 5.98 (m, 1H), 5.07- 4.91 (m, 2H), 4.72- 4.60 (m, 1H), 4.58- 4.51 (m, 1H), 4.37- 4.28 (m, 2H), 4.27- 4.19 (m, 2H), 4.18- 4.08 (m, 2H), 4.07- 4.00 (m, 2H), 4.00- 3.75 (m, 2H), 3.86- 3.73 (m, 1H), 2.49- 2.11 (m, 7H), 2.04- |
| | yl)(phenoxy) | | | 1.69 (m, 3H), 1.62- |
| | phosphoryl)- | | | 1.51 (m, 2H), 1.47- |
| | L-alaninate | | | 1.35 (m, 1H), 1.28- |
| | | | | 1.05 (m, 3H), 0.98- |
| | | | | 0.79 (m, 5H), 0.12- |
| | | | | 0.03 (m, 1H) |
| 424 | propyl(((R)- fluoro(2- (((3S,6S,7aS, 8aR,9aR)-3- (3-(4-(4- methylpipera- zin-1- yl)pyridin-3- yl)azetidine- 1-carbonyl)- 5- oxodecahydro- 1H- cyclopropa[d] pyrrolo[1,2- a]azocin-6- | 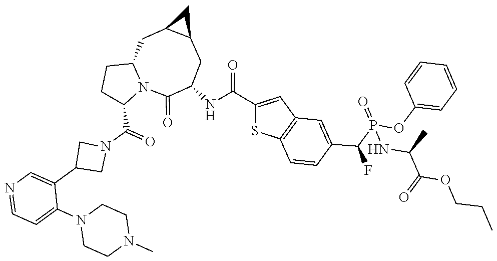 | 914.4 | 1H NMR (400 MHz, CD₃OD) δ 8.49-8.38 (m, 1H), 8.37-8.21 (m, 1H), 8.14-7.91 (m, 2H), 7.90-7.72 (m, 1H), 7.68-7.5 (m, 1H), 7.44-7.28 (m, 2H), 7.27-7.07 (m, 3H), 7.05-6.66 (m, 1H), 6.19-5.97 (m, 1H), 5.11-4.90 (m, 2H), 4.80-4.70 (m, 1H), 4.65-4.56 (m, 1H), 4.55-4.40 (m, 1H), 4.39-4.15 (m, 3H), 4.08-3.87 |
| | yl)carbamoyl) | | | (m, 3H), 3.13-2.87 |
| | benzo[b]thio- | | | (m, 3H), 2.86-2.64 |
| | phen-5- | | | (m, 3H), 2.59 (s, 2H), |
| | yl)methyl)(phe- | | | 2.50-2.39 (m, 2H), |
| | noxy)phos- | | | 2.38-2.28 (m, 4H), |
| | phoryl)-L- | | | 2.27-2.14 (m, 2H), |
| | alaninate | | | 2.02-1.63 (m, 3H), |
| | | | | 1.62-1.49 (m, 2H), |
| | | | | 1.42 (dd, J = 6.4, 10.4 |
| | | | | Hz, 1H), 1.34-1.20 |
| | | | | (m, 3H), 1.03-0.78 |
| | | | | (m, 5H), 0.07 (dd, J = |
| | | | | 5.6, 8.8 Hz, 1H) |
| 423 | propyl(((S)- (2- (((3S,6S,7aS, 8aR,9aR)-3- (3-(4- ethoxypyridin- 3- yl)azetidine- 1-carbonyl)- 5- oxodecahydro- 1H- cyclopropa[d] pyrrolo[1,2- a]azocin-6- yl)carbamoyl) benzo[b]thio- phen-5- yl)fluorometh- | 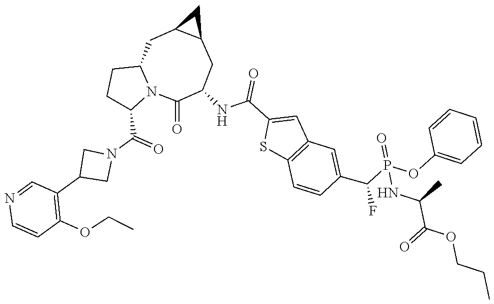 | 860.3 | 1H NMR (400 MHz, CD₃OD) δ 8.36-8.24 (m, 2H), 8.14-8.07 (m, 1H), 8.03-7.84 (m, 2H), 7.67-7.58 (m, 1H), 7.40-7.29 (m, 2H), 7.27-7.13 (m, 3H), 7.07-6.88 (m, 1H), 6.20-5.95 (m, 1H), 5.08-4.90 (m, 2H), 4.78-4.61 (m, 1H), 4.60-4.52 (m, 1H), 4.49-4.35 (m, 1H), 4.34-4.26 (m, 1H), 4.25-4.12 (m, 2H), 4.11-3.94 (m, 3H), 3.94-3.76 (m, 2H), 2.56-2.09 (m, 5H), 2.05-1.81 |
| | yl)(phenoxy) | | | (m, 2H), 1.80-1.66 |
| | phosphoryl)- | | | (m, 2H), 1.64-1.49 |
| | L-alaninate | | | (m, 2H), 1.49-1.33 |
| | | | | (m, 2H), 1.33-1.18 |
| | | | | (m, 3H), 1.11-0.80 |
| | | | | (m, 6H), 0.11-0.01 |
| | | | | (m, 1H) |
| 418 | propyl((R)- ((S)-(2- (((3S,6S,7aS, 8aR,9aR)-3- (3-(4- (dimethylami- no)pyridin-3- yl)azetidine- 1-carbonyl)- 5- oxodecahydro- 1H- cyclopropa[d] pyrrolo[1,2- a]azocin-6- yl)carbamoyl) | 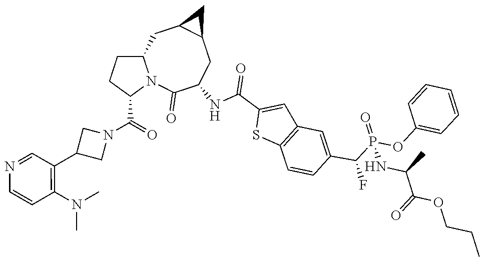 | 859.2 | 1H NMR (400 MHz, CDCl₃) δ 8.66-8.43 m, 1H), 8.34 (d, J = 5.6 Hz, 1H), 7.97 (s, 1H), 7.88-7.78 (m, 2H), 7.59-7.39 (m, 2H), 7.38-7.32 (m, 2H), 7.26-7.16 (m, 2H), 6.81 (d, J = 5.6 Hz, 1H), 6.03-5.88 (m, 1H), 5.11-4.83 (m, 2H), 4.61 (q, J = 9.2 Hz, 1H), 4.49 (t, J = 6.0 Hz, 1H), 4.43- 4.28 (m, 1H), 4.25 (d, J = 8.0 Hz, 1H), 4.19 |
| | benzo[b]thio- | | | (d, J = 6.4 Hz, 1H), |
| | phen-5- | | | 4.02-3.94 (m, 1H), |
| | yl)fluorometh- | | | 3.93-3.87 (m, 1H), |
| | yl)(phenoxy) | | | 3.86-3.78 (m, 1H), |
| | phosphoryl)- | | | 3.66-3.60 (m, 1H), |
| | L-alaninate | | | 2.76 (s, 6H), 2.38- |
| | | | | 2.28 (m, 3H), 2.16- |
| | | | | 2.02 (m, 4H), 1.75- |
| | | | | 1.71 (m, 2H), 1.56- |
| | | | | 1.45 (m, 3H), 1.27 (d, |
| | | | | J = 7.2 Hz, 3H), 1.03- |
| | | | | 0.94 (m, 1H), 0.88- |
| | | | | 0.83 (m, 3H), 0.04- |
| | | | | 0.02 (m, 1H) |
| 417 | propyl(((R)- fluoro(2- (((3S,6S,7aS, 8aR,9aR)-3- (3-(4-((S)-2- (methoxymeth- yl)azetidin- 1-yl)pyridin- 3- yl)azetidine- 1-carbonyl)- 5- oxodecahydro- 1H- cyclopropa[d] pyrrolo[1,2- a]azocin-6- yl)carbamoyl) benzo[b]thio- phen-5- yl)methyl)(phe- | 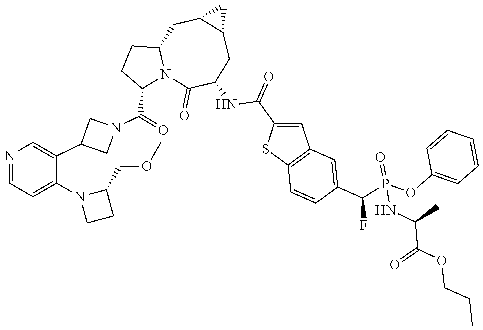 [SMILES: CCCOC(=O)[C@H](C)NP(=O)(Oc1ccccc1)[C@@H](F)c1ccc2sc(C(=O)N[C@H]3C[C@H]4C[C@H]4C[C@H]4CC[C@@H](C(=O)N5CC(c6cnccc6N6CC[C@H]6COC)C5)N4C3=O)cc2c1] | 915.4 | 1H NMR (400 MHz, CD₃OD) δ 8.50-8.25 (m, 1H), 8.15-7.88 (m, 4H), 7.63 (t, J = 8.4 Hz, 1H), 7.46- 7.26 (m, 2H), 7.26- 7.06 (m, 3H), 6.82 (dd, J = 6.4, 11.6 Hz, 1H), 6.20-5.97 (m, 1H), 5.05-4.98 (m, 1H), 4.73-4.62 (m, 1H), 4.55 (d, J = 8.0 Hz, 2H), 4.46-4.38 (m, 1H), 4.37-4.13 (m, 3H), 4.11-3.81 (m, 6H), 3.67-3.51 (m, 2H), 3.34-3.31 (m, 3H), 2.59-2.36 (m, 2H), 2.35-2.10 (m, 5H), 2.03-1.93 (m, 2H), 1.80-1.67 |
| | noxy)phos- | | | (m, 1H), 1.57-1.55 |
| | phoryl)-L- | | | (m, 2H), 1.41 (d, J = |
| | alaninate | | | 1.6 Hz, 1H), 1.23 (d, |
| | | | | J = 6.4 Hz, 3H), 0.96- |
| | | | | 0.79 (m, 5H), 0.08- |
| | | | | 0.06 (m, 1H) |
| 416 | propyl(((S)- fluoro(2- (((3S,6S,7aS, 8aR,9aR)-3- (3-(4-((S)-2- (methoxymeth- yl)azetidin- 1-yl)pyridin- 3- yl)azetidine- 1-carbonyl)- 5- oxodecahydro- 1H- cyclopropa[d] pyrrolo[1,2- a]azocin-6- yl)carbamoyl) benzo[b]thio- phen-5- yl)methyl)(phe- | 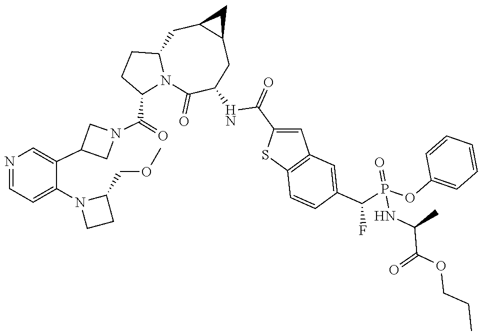 [SMILES: CCCOC(=O)[C@H](C)NP(=O)(Oc1ccccc1)[C@H](F)c1ccc2sc(C(=O)N[C@H]3C[C@@H]4C[C@@H]4C[C@H]4CC[C@@H](C(=O)N5CC(c6cnccc6N6CC[C@H]6COC)C5)N4C3=O)cc2c1] | 915.4 | 1H NMR (400 MHz, CD₃OD) δ 8.27 (d, J = 8.4 Hz, 1H), 8.11- 7.91 (m, 4H), 7.69- 7.58 (m, 1H), 7.40- 7.28 (m, 2H), 7.28- 7.05 (m, 3H), 6.81 (t, J = 6.0 Hz, 1H), 6.20- 5.98 (m, 1H), 5.05- 4.96 (m, 1H), 4.76- 4.63 (m, 1H), 4.59- 4.49 (m, 2H), 4.45- 4.36 (m, 1H), 4.35- 4.27 (m, 2H), 4.24- 4.11 (m, 1H), 4.09- 3.92 (m, 4H), 3.91- 3.76 (m, 2H), 3.66- 3.55 (m, 2H), 3.35 (s, 3H), 2.52-2.37 (m, 2H), 2.35-2.26 (m, |
| | noxy)phos- | | | 2H), 2.24-2.09 (m, |
| | phoryl)-L- | | | 3H), 2.02-1.92 (m, |
| | alaninate | | | 2H), 1.79-1.69 (m, |
| | | | | 1H), 1.62-1.52 (m, |
| | | | | 2H), 1.46-1.37 (m, |
| | | | | 1H), 1.24 (d, J = 7.2 |
| | | | | Hz, 1H), 1.07 (dd, J = |
| | | | | 2.0, 7.2 Hz, |
| | | | | 2H), 0.95-0.80 (m, |
| | | | | 5H), 0.07 (t, J = |
| | | | | 4.8 Hz, 1H) |
| 415 | propyl((R)- ((R)-(2- (((3S,6S,7aS, 8aR,9aR)-3- (3-(4- (dimethylami- no)pyridin-3- yl)azetidine- 1-carbonyl)- 5- oxodecahydro- 1H- cyclopropa[d] pyrrolo[1,2- a]azocin-6- yl)carbamoyl) benzo[b]thio- phen-5- yl)fluorometh- yl)(phenoxy) phosphoryl)- L-alaninate | 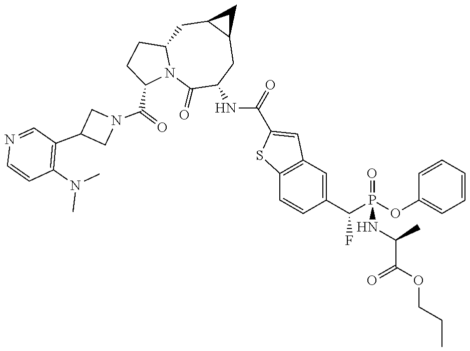 [SMILES: CCCOC(=O)[C@H](C)N[P@@](=O)(Oc1ccccc1)[C@H](F)c1ccc2sc(C(=O)N[C@H]3C[C@@H]4C[C@@H]4C[C@H]4CC[C@@H](C(=O)N5CC(c6cnccc6N(C)C)C5)N4C3=O)cc2c1] | 859.3 | ¹H NMR (400 MHz, CDCl₃) δ 8.59-8.45 (m, 1H), 8.33 (d, J = 5.6 Hz, 1H), 7.96 (s, 1H), 7.89-7.78 (m, 2H), 7.64-7.43 (m, 2H), 7.27 (s, 1H), 7.26-7.11 (m, 2H), 7.10-7.07 (m, 2H), 6.83-6.76 (m, 1H), 6.02-5.87 (m, 1H), 5.10-4.81 (m, 2H), 4.61-4.60 (m, 1H), 4.49 (t, J = 6.0 Hz, 1H), 4.43-4.27 (m, 1H), 4.22-4.11 (m, 3H), 4.08-4.02 (m, 2H), 3.65 (t, J = 10.8 Hz, 1H), 2.76-2.71 (m, 6H), 2.40-2.28 (m, 3H), 2.19-1.94 (m, 5H), 1.72-1.59 |
| | | | | (m, 3H), 1.52-1.42 |
| | | | | (m, 1H), 1.29 (dd, J = |
| | | | | 2.0, 7.2 Hz, 3H), |
| | | | | 0.92-0.90 (m, 3H), |
| | | | | 0.87-0.78 (m, 1H), |
| | | | | 0.04-0.03 (m, 1H) |
| 414 | propyl((S)- ((R)-(2- (((3S,6S,7aS, 8aR,9aR)-3- (3-(4- (dimethylami- no)pyridin-3- yl)azetidine- 1-carbnyl)- 5- oxodecahydro- 1H- cyclopropa[d] pyrrolo[1,2- a]azocin-6- yl)carbamoyl) benzo[b]thio- phen-5- yl)fluorometh- yl)(phenoxy) phosphoryl)- L-alaninate | 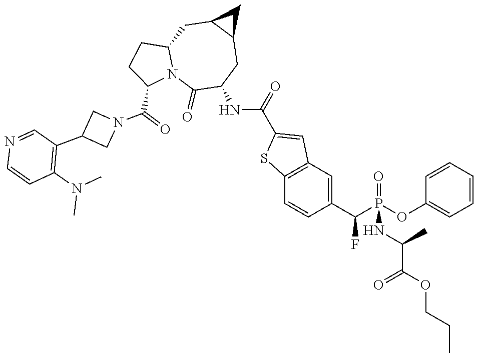 [SMILES: CCCOC(=O)[C@H](C)N[P@@](=O)(Oc1ccccc1)[C@@H](F)c1ccc2sc(C(=O)N[C@H]3C[C@@H]4C[C@@H]4C[C@H]4CC[C@@H](C(=O)N5CC(c6cnccc6N(C)C)C5)N4C3=O)cc2c1] | 859.1 | 1H NMR (400 MHz, CDCl₃) δ 8.51-8.44 (m, 1H), 8.37-8.27 (m, 1H), 7.96-7.74 (m, 3H), 7.59-7.51 (m, 1H), 7.41-7.33 (m, 2H), 7.27 (s, 2H), 7.23-7.17 (m, 1H), 6.82-6.73 (m, 1H), 6.03-5.85 (m, 1H), 5.18-4.78 (m, 2H), 4.66-4.48 (m, 2H), 4.45-4.00 (m, 7H), 3.81-3.68 (m, 1H), 2.75-2.65 (m, 6H), 2.42-2.27 (m, 3H), 2.19-1.98 (m, 4H), 1.74-1.57 (m, 3H), 1.55-1.42 (m, 1H), 1.16-1.10 (m, 3H), 1.02-0.93 (m, 1H), 0.93-0.87 (m, 3H), |
| | | | | 0.86-0.78 (m, 1H), |
| | | | | 0.05-−0.06 (m, 1H) |
| 413 | propyl(((S)- fluoro(2- (((3S,6S,7aS, 8aR,9aR)-3- (3-(4- morpholinopy- ridin-3- yl)azetidine- 1-carbonyl)- 5- oxodecahydro- 1H- cyclopropa[d] pyrrolo[1,2- a]azocin-6- yl)carbamoyl) benzo[b]thio- phen-5- yl)methyl)(phe- noxy)phos- phoryl)-L- | 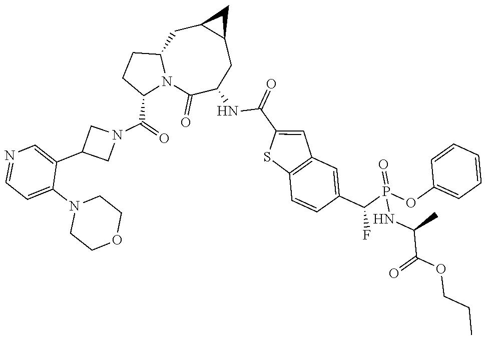 [SMILES: CCCOC(=O)[C@H](C)NP(=O)(Oc1ccccc1)[C@H](F)c1ccc2sc(C(=O)N[C@H]3C[C@@H]4C[C@@H]4C[C@H]4CC[C@@H](C(=O)N5CC(c6cnccc6N6CCOCC6)C5)N4C3=O)cc2c1] | 900.34 | 1H NMR (400 MHz, CD₃OD) δ 8.50 (d, J = 4.0 Hz, 1H), 8.35- 8.23 (m, 1H), 8.16- 8.02 (m, 2H), 8.01- 7.90 (m, 1H), 7.68- 7.60 (m, 1H), 7.40- 7.13 (m, 6H), 6.19- 5.99 (m, 1H), 5.09- 4.89 (m, 2H), 4.75 (t, J = 8.8 Hz, 1H), 4.66- 4.50 (m, 2H), 4.46- 4.29 (m, 2H), 4.23- 4.15 (m, 1H), 4.13- 4.04 (m, 1H), 4.03- 3.82 (m, 3H), 3.80- 3.76 (m, 4H), 3.41- 3.33 (m, 3H), 2.50- 2.38 (m, 1H), 2.35- 2.13 (m, 4H), 2.03- |
| | alaninate | | | 1.89 (m, 2H), 1.79- |
| | | | | 1.68 (m, 1H), 1.62- |
| | | | | 1.52 (m, 2H), 1.47- |
| | | | | 1.36 (m, 1H), 1.24 (d, |
| | | | | J = 7.2 Hz, 1H), 1.12- |
| | | | | 1.05 (m, 2H), 0.97- |
| | | | | 0.87 (m, 5H), 0.09- |
| | | | | 0.06 (m, 1H) |
| 412 | propyl(((S)- (2- (((3S,6S,7aS, 8aR,9aR)-3- (3-(4- (azetidin-1- yl)pyrimidin- 5- yl)azetidine- 1-carbonyl)- 5- oxodecahydro- 1H- cyclopropa[d] pyrrolo[1,2- a]azocin-6- yl)carbamoyl) | 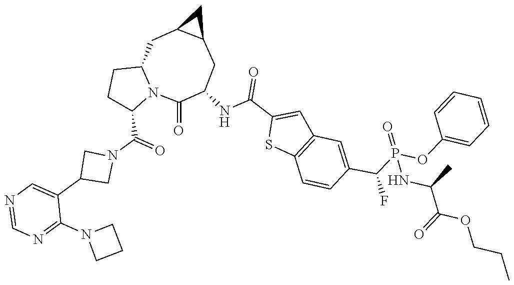 | 872.5 | 1H NMR (400 MHz, CDCl₃) δ 9.05-8.55 (m, 1H), 8.53-8.22 (m, 2H), 7.95-7.94 (m, 1H), 7.86-7.76 (m, 1H), 7.60-7.51 (m, 1H), 7.46-7.32 (m, 1H), 7.24-7.06 (m, 5H), 5.99-5.87 (m, 1H), 5.22-5.04 (m, 2H), 4.49-4.37 (m, 4H), 4.36-4.16 (m, 3H), 4.09-4.06 (m, 1H), 4.04-4.01 (m, 1H), 3.99-3.87 (m, 1H), 3.85-3.76 (m, 1H), 3.69-3.61 |
| | benzo[b]thio- | | | (m, 2H), 2.57-2.49 |
| | phen-5- | | | (m, 3H), 2.29-2.04 |
| | yl)fluorometh- | | | (m, 3H), 1.96-1.81 |
| | yl)(phenoxy) | | | (m, 2H), 1.63-1.33 |
| | phosphoryl)- | | | (m, 4H), 1.29-1.26 |
| | L-alaninate | | | (m, 4H), 0.91-0.84 |
| | | | | (m, 3H), 0.83-0.68 |
| | | | | (m, 2H), −0.02-−0.08 |
| | | | | (m, 1H). |
| 409 | propyl(((R)- fluoro(2- (((3S,6S,7aS, 8aR,9aR)-3- (3-(4- morpholinopy- ridin-3- yl)azetidine- 1-carbonyl)- 5- oxodecahydro- 1H- cyclopropa[d] pyrrolo[1,2- a]azocin-6- yl)carbamoyl) | 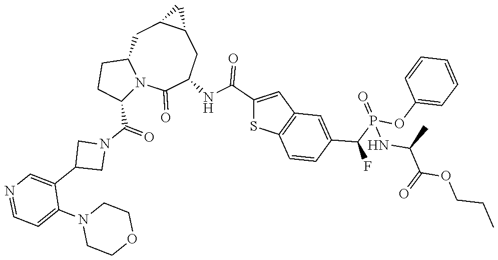 | 901.1 | 1H NMR (400 MHz, CD₃OD) δ 8.54-8.48 (m, 1H), 8.31 (dd, J = 6.4, 17.2 Hz, 1H), 8.07 (d, J = 13.6 Hz, 2H), 7.99-7.91 (m, 1H), 7.65 (d, J = 8.4 Hz, 1H), 7.37-7.25 (m, 3H), 7.24-7.13 (m, 3H), 6.18-6.02 (m, 1H), 5.06-4.99 (m, 1H), 4.76 (t, J = 8.8 Hz, 1H), 4.67- 4.51 (m, 2H), 4.45- 4.29 (m, 2H), 4.24- 4.16 (m, 1H), 4.13- |
| | benzo[b]thio- | | | 4.04 (m, 1H), 4.01- |
| | phen-5- | | | 3.91 (m, 1H), 3.90 (s, |
| | yl)methyl)(phe- | | | 1H), 3.85-3.76 (m, |
| | noxy)phos- | | | 5H), 3.43-3.33 (m, |
| | phoryl)-L- | | | 4H), 2.51-2.38 (m, |
| | alaninate | | | 1H), 2.36-2.25 (m, |
| | | | | 2H), 2.24-2.12 (m, |
| | | | | 2H), 2.04-1.93 (m, |
| | | | | 2H), 1.80-1.69 (m, |
| | | | | 1H), 1.58-1.49 (m, |
| | | | | 2H), 1.46-1.37 (m, |
| | | | | 1H), 1.26-1.19 (m, |
| | | | | 3H), 0.98-0.90 (m, |
| | | | | 1H), 0.88-0.82 (m, |
| | | | | 4H), 0.11-0.05 (m, |
| | | | | 1H) |
| 430 | propyl((R)- ((R)-(2- (((3S,6S,7aS, 8aR,9aR)-3- ((6S,7R)-7- cyano-6- (pyridin-3- yl)-4- azaspiro[2.4] heptane-4- carbonyl)-5- oxodecahydro- 1H- cyclopropa[d] pyrrolo[1,2- a]azocin-6- yl)carbamoyl) benzo[b]thio- phen-5- yl)fluorometh- yl)(phenoxy) phosphoryl)- L-alaninatel | 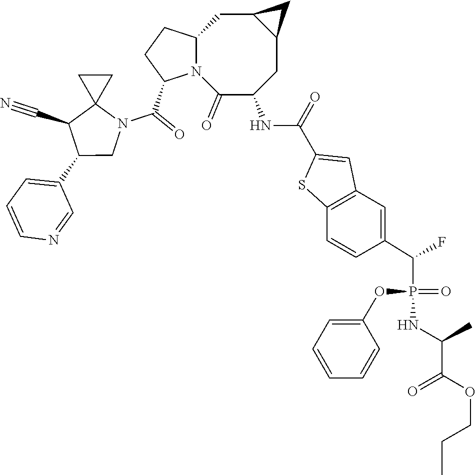 [SMILES: CCCOC(=O)[C@H](C)N[P@](=O)(Oc1ccccc1)[C@@H](F)c1ccc2sc(C(=O)N[C@H]3C[C@@H]4C[C@@H]4C[C@H]4CC[C@@H](C(=O)N5C[C@H](c6cccnc6)[C@@H](C#N)C56CC6)N4C3=O)cc2c1] | 881.4 | ¹H NMR (400 MHz, DMSO-d₆) δ 8.94- 8.87 (m, 1H), 8.72- 8.66 (m, 1H), 8.55- 8.49 (m, 1H), 8.34- 8.27 (m, 1H), 8.11- 8.02 (m, 2H), 7.97- 7.89 (m, 1H), 7.65- 7.57 (m, 1H), 7.45- 7.39 (m, 1H), 7.39- 7.32 (m, 2H), 7.21- 7.11 (m, 3H), 6.27- 6.04 (m, 2H), 5.01- 4.89 (m, 1H), 4.63- 4.45 (m, 2H), 4.20- 3.99 (m, 2H), 3.89- 3.69 (m, 5H), 2.39- 2.31 (m, 1H), 2.21- 1.96 (m, 5H), 1.90- 1.80 (m, 2H), 1.73- 1.57 (m, 2H), 1.47- 1.27 (m, 3H), 1.19- 1.07 (m, 3H), 0.86- 0.61 (m, 7H), 0.02- 0.00 (m, 1H). |
| | or | Or | | |
| | propyl((S)- ((R)-(2- (((3S,6S,7aS, 8aR,9aR)-3- ((6S,7R)-7- cyano-6- (pyridin-3- yl)-4- azaspiro[2.4] heptane-4- carbonyl)-5- oxodecahydro- 1H- cyclopropa[d] pyrrolo[1,2- a]azocin-6- yl)carbamoyl) benzo[b]thio- phen-5- yl)fluorometh- yl)(phenoxy) phosphoryl)- L-alaninate | 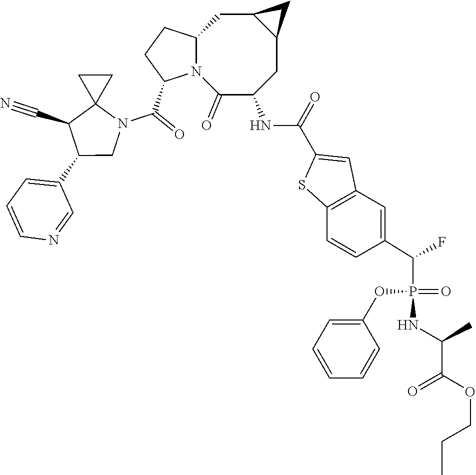 [SMILES: CCCOC(=O)[C@H](C)N[P@@](=O)(Oc1ccccc1)[C@@H](F)c1ccc2sc(C(=O)N[C@H]3C[C@@H]4C[C@@H]4C[C@H]4CC[C@@H](C(=O)N5C[C@H](c6cccnc6)[C@@H](C#N)C56CC6)N4C3=O)cc2c1] | | |
| | or | Or | | |
| | propyl((R)- ((R)-(2- (((3S,6S,7aS, 8aR,9aR)-3- ((6R,7S)-7- cyano-6- (pyridin-3- yl)-4- azaspiro[2.4] heptane-4- carobnyl)-5- oxodecahydro- 1H- cyclopropa[d] pyrrolo[1,2- a]azocin-6- yL)carbamoyl) benzo[b]thio- phen-5- yl)fluorometh- yl)(phenoxy) phosphoryl)- L-alaninate | 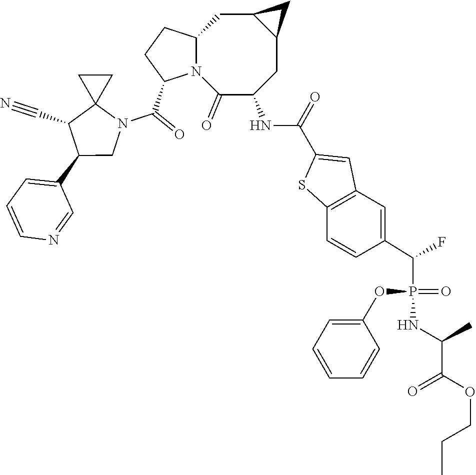 [SMILES: CCCOC(=O)[C@H](C)N[P@](=O)(Oc1ccccc1)[C@@H](F)c1ccc2sc(C(=O)N[C@H]3C[C@@H]4C[C@@H]4C[C@H]4CC[C@@H](C(=O)N5C[C@@H](c6cccnc6)[C@H](C#N)C56CC6)N4C3=O)cc2c1] | | |
| | or | Or | | |
| | propyl((S)- ((R)-(2- (((3S,6S,7aS, 8aR,9aR)-3- ((6R,7S)-7- cyano-6- (pyridin-3- yl)-4- azaspiro[2.4] heptane-4- carbonyl)-5- oxodecahydro- 1H- cyclopropa[d] pyrrolo[1,2- a]azocin-6- yl)carbamoyl) benzo[b]thio- phen-5- yl)fluorometh- yl)(phenoxy) phosphoryl)- L-alaninate | 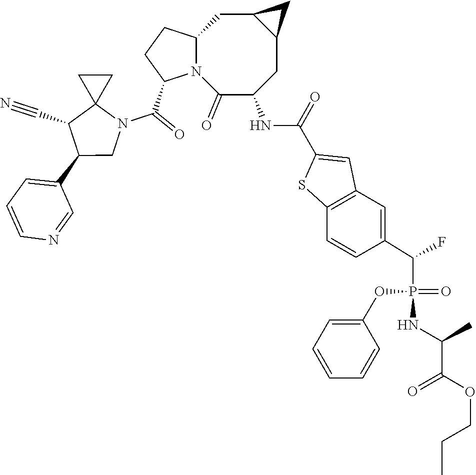 [SMILES: CCCOC(=O)[C@H](C)N[P@@](=O)(Oc1ccccc1)[C@@H](F)c1ccc2sc(C(=O)N[C@H]3C[C@@H]4C[C@@H]4C[C@H]4CC[C@@H](C(=O)N5C[C@@H](c6cccnc6)[C@H](C#N)C56CC6)N4C3=O)cc2c1] | | |
| | | Pyrrolidine building block SFC | | |
| | | peak 1 and CFTE-28B were used | | |
| | | for synthesis and biological | | |
| | | testing | | |
| 431 | propyl((S)- ((R)-(2- (((3S,6S,7aS, 8aR,9aR)-3- ((6S,7R)-7- cyano-6- (pyridin-3- yl)-4- azaspiro[2.4] heptane-4- carbonyl)-5- oxodecahydro- 1H- cyclopropa[d] pyrrolo[1,2- a]azocin-6- yl)carbamoyl) benzo[b]thio- phen-5- yl)fluorometh- yl)(phenoxy) phosphoryl)- L-alaninate | 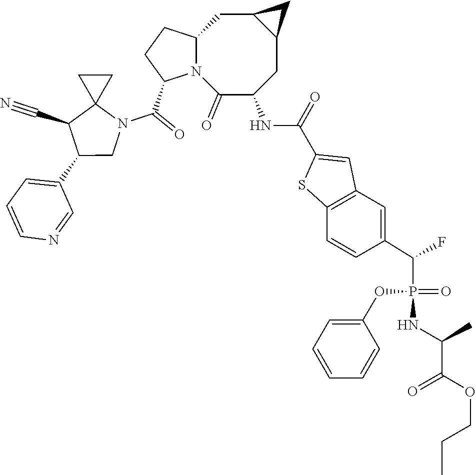 [SMILES: CCCOC(=O)[C@H](C)N[P@@](=O)(Oc1ccccc1)[C@@H](F)c1ccc2sc(C(=O)N[C@H]3C[C@@H]4C[C@@H]4C[C@H]4CC[C@@H](C(=O)N5C[C@H](c6cccnc6)[C@@H](C#N)C56CC6)N4C3=O)cc2c1] | 881.6 | ¹H NMR (400 MHz, DMSO-d₆) δ 8.92 (d, J = 7.6 Hz, 1H), 8.83 (s, 1H), 8.66 (d, J = 4.0 Hz, 1H), 8.31 (s, 1H), 8.23-8.16 (m, 1H), 8.11-8.04 (m, 2H), 7.69-7.57 (m, 2H), 7.40-7.32 (m, 2H), 7.22-7.15 (m, 3H), 6.29-6.10 (m, 2H), 5.00-4.90 (m, 1H), 4.60-4.54 (m, 2H), 4.18-4.07 (m, 2H), 3.97-3.89 (m, 3H), 3.84-3.74 (m, 2H), 2.41-2.31 (m, 1H), 2.21-1.98 (m, 5H), 1.91-1.80 (m, 2H), 1.75-1.58 (m, 2H), 1.56-1.46 (m, 2H), 1.36-1.27 (m, 1H), 1.14 (d, J = 7.1 Hz, 3H), 0.87-0.73 (m, 6H), 0.72-0.63 (m, 1H), 0.03-0.00 (m, 1H). |
| | or | Or | | |
| | propyl((R)- ((R)-(2- (((3S,6S,7aS, 8aR,9aR)-3- ((6S,7R)-7- cyano-6- (pyridin-3- yl)-4- azaspiro[2.4] heptane-4- carobnyl)-5- oxodecahydro- 1H- cyclopropa[d] pyrrolo[1,2- a]azocin-6- yl)carbamoyl) benzo[b]thio- phen-5- yl)fluorometh- yl)(phenoxy) phosphoryl)- L-alaninate | 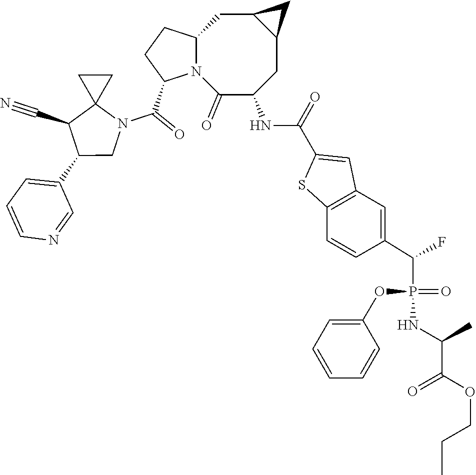 [SMILES: CCCOC(=O)[C@H](C)N[P@](=O)(Oc1ccccc1)[C@@H](F)c1ccc2sc(C(=O)N[C@H]3C[C@@H]4C[C@@H]4C[C@H]4CC[C@@H](C(=O)N5C[C@H](c6cccnc6)[C@@H](C#N)C56CC6)N4C3=O)cc2c1] | | |
| | or | Or | | |
| | propyl((S)- ((R)-(2- (((3S,6S,7aS, 8aR,9aR)-3- ((6R,7S)-7- cyano-6- (pyridin-3- yl)-4- azaspiro[2.4] heptane-4- carbonyl)-5- oxodecahydro- 1H- cyclopropa[d] pyrrolo[1,2- a]azocin-6- yl)carbamoyl) benzo[b]thio- phen-5- yl)fluorometh- yl)(phenoxy) phosphoryl)- L-alaninate | 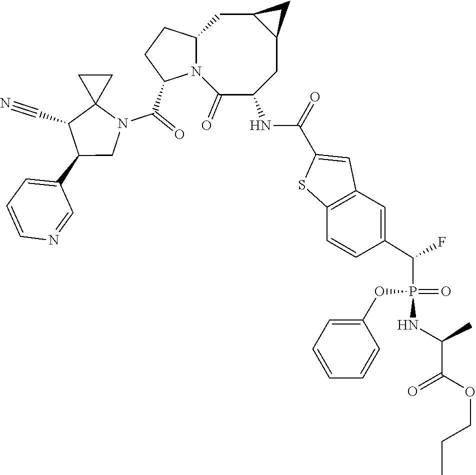 [SMILES: CCCOC(=O)[C@H](C)N[P@@](=O)(Oc1ccccc1)[C@@H](F)c1ccc2sc(C(=O)N[C@H]3C[C@@H]4C[C@@H]4C[C@H]4CC[C@@H](C(=O)N5C[C@@H](c6cccnc6)[C@H](C#N)C56CC6)N4C3=O)cc2c1] | | |
| | or | Or | | |
| | propyl((R)- ((R)-(2- (((3S,6S,7aS, 8aR,9aR)-3- ((6R,7S)-7- cyano-6- (pyridin-3- yL)-4- azaspiro[2.4] heptane-4- carobnyl)-5- oxodecahydro- 1H- cyclopropa[d] pyrrolo[1,2- a]azocin-6- yl)carbamoyl) benzo[b]thio- phen-5- yl)fluorometh- yl)(phenoxy) phosphoryl)- L-alaninate | 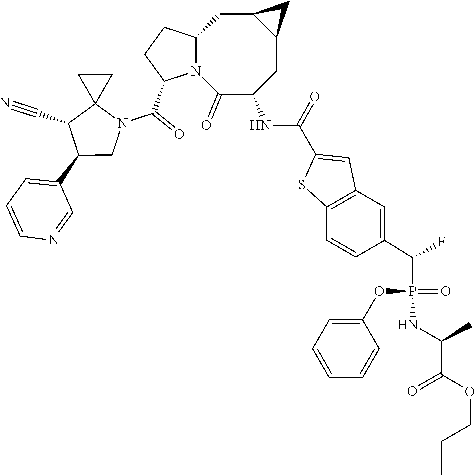 [SMILES: CCCOC(=O)[C@H](C)N[P@](=O)(Oc1ccccc1)[C@@H](F)c1ccc2sc(C(=O)N[C@H]3C[C@@H]4C[C@@H]4C[C@H]4CC[C@@H](C(=O)N5C[C@@H](c6cccnc6)[C@H](C#N)C56CC6)N4C3=O)cc2c1] | | |
| | | Pyrrolidine building block SFC | | |
| | | peak 1 and CFTE-28A were used | | |
| | | for synthesis and biological | | |
| | | testing | | |
| 434 | propyl((S)- ((R)-(2- (((3S,6S,7aS, 8aR,9aR)-3- ((S)-6-(4- (dimethylami- no)pyridin-3- yl)-4- azaspiro[2.4] heptane-4- carbonyl)-5- oxodecahydro- 1H- cyclopropa[d] pyrrolo[1,2- a]azocin-6- yl)carbamoyl) benzo[b]thio- phen-5- yl)fluorometh- yl)(phenoxy) phosphoryl)- L-alaninate | 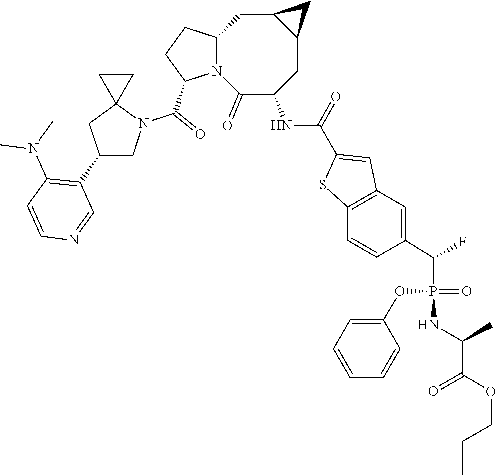 [SMILES: CCCOC(=O)[C@H](C)N[P@@](=O)(Oc1ccccc1)[C@@H](F)c1ccc2sc(C(=O)N[C@H]3C[C@@H]4C[C@@H]4C[C@H]4CC[C@@H](C(=O)N5C[C@H](c6cnccc6N(C)C)CC56CC6)N4C3=O)cc2c1] | 899.7 | ¹H NMR (400 MHz, DMSO-d₆) δ 8.95- 8.87 (m, 1H), 8.41 (s, 1H), 8.31 (s, 1H), 8.22-8.18 (m, 1H), 8.10-8.03 (m, 2H), 7.62-7.57 (m, 1H), 7.40-7.33 (m, 2H), 7.21-7.14 (m, 3H), 6.95-6.90 (m, 1H), 6.28-6.10 (m, 2H), 4.98-4.90 (m, 1H), 4.57-4.51 (m, 1H), 4.18-4.08 (m, 2H), 3.98-3.90 (m, 2H), 3.89-3.83 (m, 1H), 3.81-3.71 (m, 2H), 2.76 (s, 6H), 2.40- 2.32 (m, 1H), 2.20- 2.10 (m, 3H), 2.09- 1.94 (m, 4H), 1.90- 1.77 (m, 2H), 1.68- 1.57 (m, 2H), 1.55- 1.46 (m, 2H), 1.42- 1.34 (m, 1H), 1.16- 1.10 (m, 3H), 0.86- 0.72 (m, 5H), 0.52- 0.42 (m, 2H), −0.00- −0.03 (m, 1H). |
| | or | Or | | |
| | propyl((R)- ((R)-(2- (((3S,6S,7aS, 8aR,9aR)-3- ((S)-6-(4- (dimethylami- no)pyridin-3- yl)-4- azaspiro[2.4] heptane-4- carbonyl)-5- oxodecahydro- 1H- cyclopropa[d] pyrrolo[1,2- a]azocin-6- yl)carbamoyl) benzo[b]thio- phen-5- yl)fluorometh- yl)(phenoxy) phosphoryl)- L-alaninate | 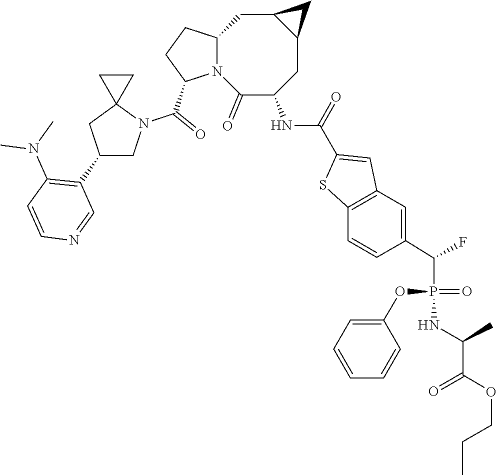 [SMILES: CCCOC(=O)[C@H](C)N[P@](=O)(Oc1ccccc1)[C@@H](F)c1ccc2sc(C(=O)N[C@H]3C[C@@H]4C[C@@H]4C[C@H]4CC[C@@H](C(=O)N5C[C@H](c6cnccc6N(C)C)CC56CC6)N4C3=O)cc2c1] | | |
| | or | Or | | |
| | propyl((S)- ((R)-(2- (((3S,6S,7aS, 8aR,9aR)-3- ((R)-6-(4- (dimethylami- no)pyridin-3- yl)-4- azaspiro[2.4] heptane-4- carbonyl)-5- oxodecahydro- 1H- cyclopropa[d] pyrrolo[1,2- a]azocin-6- yl)carbamoyl) benzo[b]thio- phen-5- yl)fluorometh- yl)(phenoxy) phosphoryl)- L-alaninate | 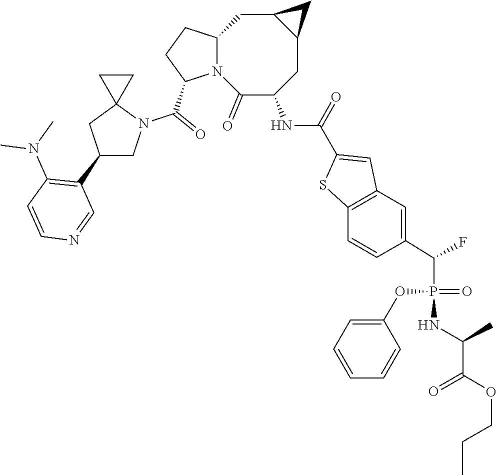 [SMILES: CCCOC(=O)[C@H](C)N[P@@](=O)(Oc1ccccc1)[C@@H](F)c1ccc2sc(C(=O)N[C@H]3C[C@@H]4C[C@@H]4C[C@H]4CC[C@@H](C(=O)N5C[C@@H](c6cnccc6N(C)C)CC56CC6)N4C3=O)cc2c1] | | |
| | or | Or | | |
| | propyl((R)- ((R)-(2- (((3S,6S,7aS, 8aR,9aR)-3- ((R)-6-(4- (dimethylami- no)pyridin-3- yl)-4- azaspiro[2.4] heptane-4- carobnyl)-5- oxodecahydro- 1H- cyclopropa[d] pyrrolo[1,2- a]azocin-6- yl)carbamoyl) benzo[b]thio- phen-5- yl)fluorometh- yl)(phenoxy) phosphoryl)- L-alaninate | 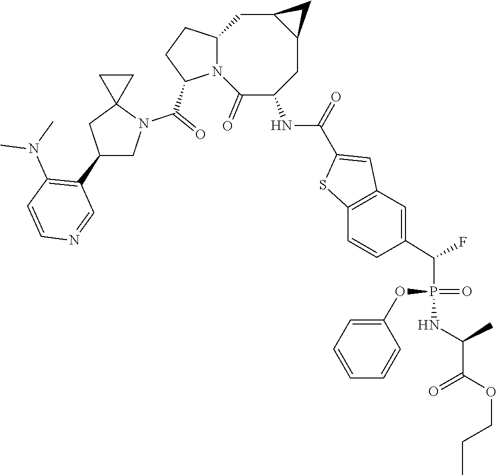 [SMILES: CCCOC(=O)[C@H](C)N[P@](=O)(Oc1ccccc1)[C@@H](F)c1ccc2sc(C(=O)N[C@H]3C[C@@H]4C[C@@H]4C[C@H]4CC[C@@H](C(=O)N5C[C@@H](c6cnccc6N(C)C)CC56CC6)N4C3=O)cc2c1] | | |
| | | Pyrrolidine + Core SFC peak 2 | | |
| | | and CFTE-28A were used for | | |
| | | synthesis and biological testing | | |
| 432 | propyl((S)- ((R)-(2- (((3S,6S,7aS, 8aR,9aR)-3- ((R)-6-(4- (dimethylami- no)pyridin-3- yl)-4- azaspiro[2.4] heptane-4- carbonyl)-5- oxodecahydro- 1H- cyclopropan[d] pyrrolo[1,2- a]azocin-6- yl)carbamoyl) benzo[b]thio- phen-5- yl)fluorometh- yl)(phenoxy) phosphoryl)- L-alaninate | 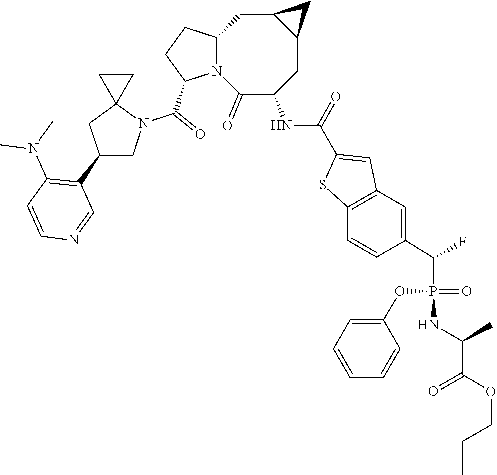 [SMILES: CCCOC(=O)[C@H](C)N[P@@](=O)(Oc1ccccc1)[C@@H](F)c1ccc2sc(C(=O)N[C@H]3C[C@@H]4C[C@@H]4C[C@H]4CC[C@@H](C(=O)N5C[C@@H](c6cnccc6N(C)C)CC56CC6)N4C3=O)cc2c1] | 899.3 | ¹H NMR (400 MHz, DMSO-d₆) δ 8.88 (d, J = 7.7 Hz, 1H), 8.46 (s, 1H), 8.31 (s, 1H), 8.23 (d, J = 5.5 Hz, 1H), 8.15-8.05 (m, 2H), 7.60 (d, J = 8.7 Hz, 1H), 7.39-7.32 (m, 2H), 7.23-7.15 (m, 3H), 6.91 (d, J = 5.6 Hz, 1H), 6.28- 6.12 (m, 2H), 4.99- 4.88 (m, 1H), 4.58- 4.43 (m, 2H), 4.19- 4.08 (m, 1H), 4.00- 3.88 (m, 2H), 3.81- 3.70 (m, 2H), 3.64- 3.57 (m, 1H), 2.70 (s, 6H), 2.38-2.27 (m, 2H), 2.18-2.10 (m, 1H), 2.09-1.93 (m, 5H), 1.90-1.79 (m, 2H), 1.68-1.55 (m, 2H), 1.54-1.45 (m, 2H), 1.41-1.30 (m, 1H), 1.13 (d, J = 7.1 Hz, 3H), 0.87-0.78 (m, 4H), 0.78-0.70 (m, 1H), 0.55-0.43 |
| | or | Or | | (m, 2H), 0.02-−0.00 |
| | propyl((R)- ((R)-(2- (((3S,6S,7aS, 8aR,9aR)-3- ((R)-6-(4- (dimethylami- no)pyridin-3- yl)-4- azaspiro[2.4] heptane-4- carbonyl)-5- oxodecahydro- 1H- cyclopropa[d] pyrrolo[1,2- a]azocin-6- yl)carbamoyl) benzo[b]thio- phen-5- yl)fluorometh- yl)(phenoxy) phosphoryl)- L-alaninate | 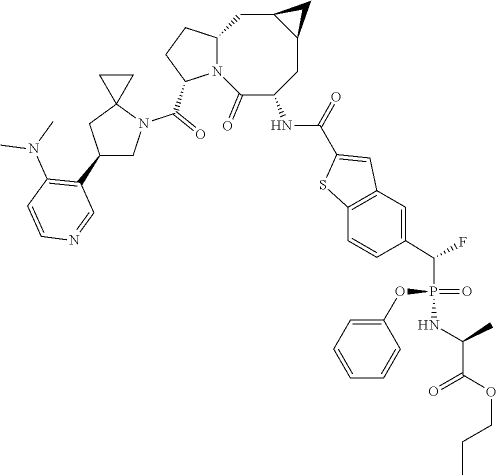 [SMILES: CCCOC(=O)[C@H](C)N[P@](=O)(Oc1ccccc1)[C@@H](F)c1ccc2sc(C(=O)N[C@H]3C[C@@H]4C[C@@H]4C[C@H]4CC[C@@H](C(=O)N5C[C@@H](c6cnccc6N(C)C)CC56CC6)N4C3=O)cc2c1] | | (m, 1H). |
| | or | Or | | |
| | propyl((S)- ((R)-(2- (((3S,6S,7aS, 8aR,9aR)-3- ((S)-6-(4- (dimethylami- no)pyridin-3- yl)-4- azaspiro[2.4] heptane-4- carbonyl)-5- oxodecahydro- 1H- cyclopropa[d] pyrrolo[1,2- a]azocin-6- yl)carbamoyl) benzo[b]thio- phen-5- yl)fluorometh- yl)(phenoxy) phosphoryl)- L-alaninate | 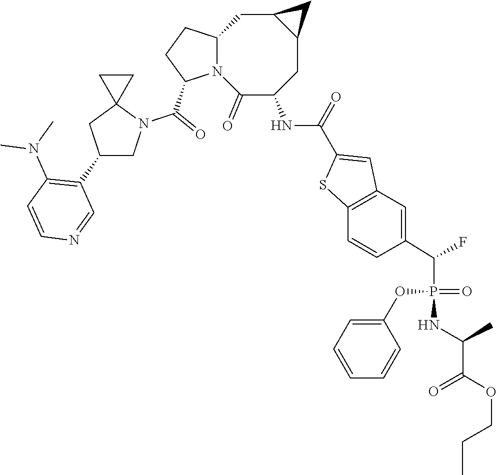 [SMILES: CCCOC(=O)[C@H](C)N[P@@](=O)(Oc1ccccc1)[C@@H](F)c1ccc2sc(C(=O)N[C@H]3C[C@@H]4C[C@@H]4C[C@H]4CC[C@@H](C(=O)N5C[C@H](c6cnccc6N(C)C)CC56CC6)N4C3=O)cc2c1] | | |
| | or | Or | | |
| | propyl((R)- ((R)-(2- (((3S,6S,7aS, 8aR,9aR)-3- ((S)-6-(4- (dimethylami- no)pyridin-3- yl)-4- azaspiro[2.4] heptane-4- carobnyl)-5- oxodecahydro- 1H- cylcopropa[d] pyrrolo[1,2- a]azocin-6- yL)carbamoyl) benzo[b]thio- phen-5- yl)fluorometh- yl)(phenoxy) phosphoryL)- L-alaninate | 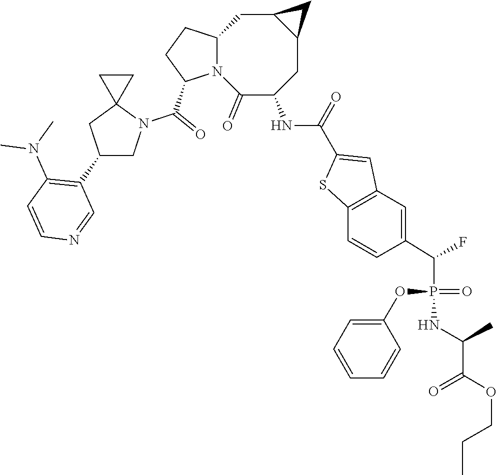 [SMILES: CCCOC(=O)[C@H](C)N[P@](=O)(Oc1ccccc1)[C@@H](F)c1ccc2sc(C(=O)N[C@H]3C[C@@H]4C[C@@H]4C[C@H]4CC[C@@H](C(=O)N5C[C@H](c6cnccc6N(C)C)CC56CC6)N4C3=O)cc2c1] | | |
| | | Pyrrolidine + Core SFC peak 1 | | |
| | | and CFTE-28A were used for | | |
| | | synthesis and biological testing | | |
| 242 | propyl(((2- (((3S,6S,7aR, 8aS,9aR)-3- (3-(4- methylpyridin- 3- yl)azetidine- 1-carbonyl)- 5- oxodecahydro- 1H- cyclopropa[d] pyrrolo[1,2- a]azocin-6- yl)carbamoyl) benzo[b]thio- phen-5- | 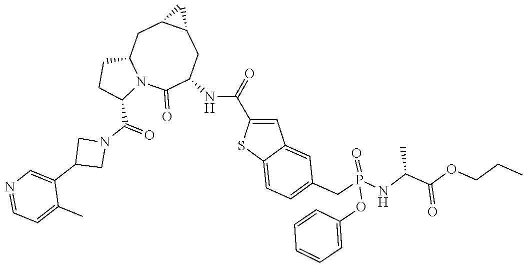 [SMILES: CCCOC(=O)[C@@H](C)NP(=O)(Cc1ccc2sc(C(=O)N[C@H]3C[C@H]4C[C@H]4C[C@H]4CC[C@@H](C(=O)N5CC(c6cnccc6C)C5)N4C3=O)cc2c1)Oc1ccccc1] | 812.3 [M + H]⁺ | ¹H NMR (400 MHz, DMSO-d₆) δ 8.88- 8.77 (m, 1H), 8.59- 8.49 (m, 1H), 8.38- 8.28 (m, 1H), 8.25- 8.16 (m, 1H), 7.98- 7.85 (m, 2H), 7.48- 7.41 (m, 1H), 7.37- 7.29 (m, 2H), 7.22- 7.09 (m, 4H), 5.77- 5.54 (m, 1H), 4.86- 4.75 (m, 1H), 4.66- 4.58 (m, 0.5H), 4.49- 4.43 (m, 0.5H), 4.37- 3.95 (m, 6H), 3.92- 3.74 (m, 3H), 3.49- 3.40 (m, 2H), 2.28- |
| | yl)methyl)(phe- | | | 2.14 (m, 5H), 2.11- |
| | noxy)phos- | | | 1.98 (m, 2H), 1.95- |
| | phoryl)-L- | | | 1.83 (m, 2H), 1.76- |
| | alaninate | | | 1.62 (m, 1H), 1.56- |
| | | | | 1.41 (m, 3H), 1.36- |
| | | | | 1.14 (m, 2H), 1.10- |
| | | | | 0.97 (m, 3H), 0.85- |
| | | | | 0.72 (m, 4H), 0.10- |
| | | | | 0.01 (m, 1H). |
| 285 | propyl 2- (((fluoro(2- (((3S,6S,7aS, 8aR,9aR)-3- (3-(4- methylpyridin- 3- yl)azetidine- 1-carbonyl)- 5- oxodecahydro- 1H- cyclopropa[d] pyrrolo[1,2- a]azocin-6- | 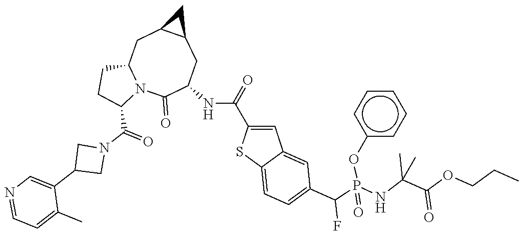 | 844.1 [M + H]⁺ | ¹H NMR (400 MHz, CD₃OD) δ 8.61-8.49 (m, 1H), 8.33-8.22 (m, 1H), 8.13-7.86 (m, 3H), 7.66-7.56 (m, 1H), 7.35-7.09 (m, 6H), 6.24-6.02 (m, 1H), 5.07-4.98 (m, 1H), 4.81-4.42 (m, 3H), 4.42-4.26 (m, 2H), 4.22-3.94 (m, 4H), 2.51-2.37 (m, 1H), 2.36-2.07 (m, 7H), 2.04-1.87 (m, 2H), 1.79-1.68 |
| | yl)carbamoyl) | | | (m, 1H), 1.64-1.53 |
| | benzo[b]thio- | | | (m, 2H), 1.48-1.27 |
| | phen-5- | | | (m, 7H), 1.01-0.79 |
| | yl)methyl)(phe- | | | (m, 5H), 0.10-0.02 |
| | noxy)phos- | | | (m, 1H). |
| | phoryl)amino)- | | | |
| | 2- | | | |
| | methylpropan- | | | |
| | oate | | | |
| 297 | propyl ((fluoro(2- (((3S,6S,7aS, 8aR,9aR)-5- oxo-3- ((1S,5S)-2- (pyridin-3- yl)-2,6-diaza- bicyclo[3.2.0] heptane- 6- carbonyl)deca- hydro-1H- cyclopropa[d] pyrrolo[1,2- a]azocin-6- | 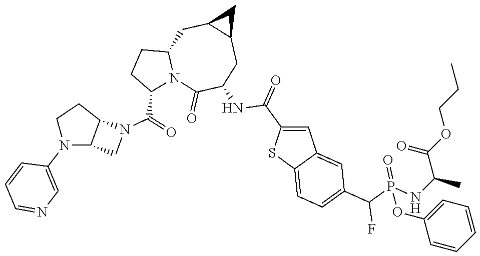 [SMILES: CCCOC(=O)[C@@H](C)NP(=O)(Oc1ccccc1)C(F)c1ccc2sc(C(=O)N[C@H]3C[C@@H]4C[C@@H]4C[C@H]4CC[C@@H](C(=O)N5C[C@H]6[C@@H]5CCN6c5cccnc5)N4C3=O)cc2c1] | 857.3 [M + H]⁺ | ¹H NMR (400 MHz, CD₃OD) δ 8.11-7.99 (m, 2H), 7.98-7.81 (m, 3H), 7.63-7.55 (m, 1H), 7.34-7.04 (m, 7H), 6.14-5.94 (m, 1H), 5.26-4.94 (m, 2H), 4.69-4.55 (m, 2H), 4.39-4.33 (m, 0.6H), 4.28-4.16 (m, 1H), 4.07-3.99 (m, 0.4H), 3.97-3.74 (m, 4H), 3.73-3.63 (m, 0.7H), 3.47-3.30 (m, 1H), 2.98-2.88 (m, 0.3H), 2.46-1.97 |
| | yl)carbamoyl) | | | (m, 7H), 1.97-1.75 |
| | benzo[b]thio- | | | (m, 2H), 1.74-1.62 |
| | phen-5- | | | (m, 5H), 1.56-1.32 |
| | yl)methyl)(phe- | | | (m, 3H), 1.21-1.13 |
| | noxy)phos- | | | (m, 2H), 1.04-0.72 |
| | phoryl)-L- | | | (m, 6H), 0.04-−0.05 |
| | alaninate | | | (m, 1H). |
| 298 | propyl ((fluoro(2- (((3S,6S,7aS, 8aR,9aR)-5- oxo-3- ((1R,5R)-2- (pyridin-3- yl)-2,6-diaza- bicyclo[3.2.0] heptane- 6- carbonyl)deca- hydro-1H- cyclopropa[d] pyrrolo[1,2- a]azocin-6- | 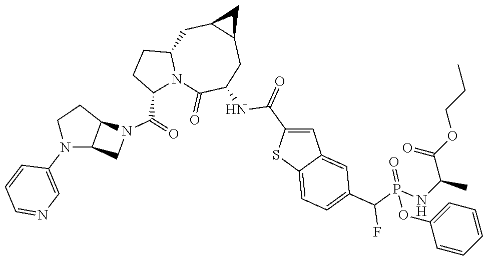 [SMILES: CCCOC(=O)[C@@H](C)NP(=O)(Oc1ccccc1)C(F)c1ccc2sc(C(=O)N[C@H]3C[C@@H]4C[C@@H]4C[C@H]4CC[C@@H](C(=O)N5C[C@@H]6[C@H]5CCN6c5cccnc5)N4C3=O)cc2c1] | 857.3 [M + H]⁺ | ¹H NMR (400 MHz, CD₃OD) δ 8.17-8.06 (m, 1H), 8.04-7.78 (m, 4H), 7.69-7.57 (m, 1H), 7.39-7.30 (m, 2H), 7.30-7.05 (m, 5H), 6.19-5.98 (m, 1H), 5.65-5.59 (m, 0.4H), 5.09-4.98 (m, 1.6H), 4.70-4.61 (m, 1H), 4.61-4.55 (m, 0.5H), 4.44-4.22 (m, 2H), 4.17-4.03 (m, 1H), 4.01-3.77 (m, 4H), 3.53-3.34 (m, 1.5H), 2.56-2.26 |
| | yl)carbamoyl) | | | (m, 4H), 2.25-2.12 |
| | benzo[b]thio- | | | (m, 2H), 2.11-1.89 |
| | phen-5- | | | (m, 3H), 1.80-1.67 |
| | yl)methyl)(phe- | | | (m, 1H), 1.62-1.34 |
| | noxy)phos- | | | (m, 3H), 1.26-1.19 |
| | phoryl)-L- | | | (m, 2H), 1.09-0.80 |
| | alaninate | | | (m, 6H), 0.10-0.03 |
| | | | | (m, 1H). |
| 307 | propyl ((fluoro(2- (((3S,6S,7aS, 8aR,9aR)-3- (3-(4- methylpyridin- 3- yl)azetidine-1- carbonyl)- 5- oxodecahydro- 1H- cyclopropa[d] pyrrolo[1,2- a]azocin-6- | 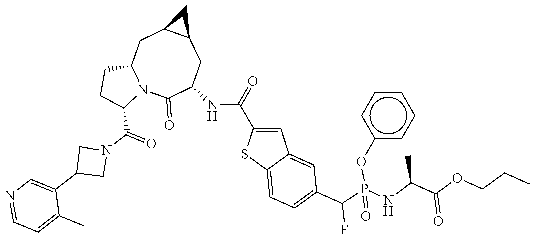 | 830.1 [M + H]⁺ | ¹H NMR (400 MHz, DMSO-d₆) δ 8.98- 8.85 (m, 1H), 8.61- 8.45 (m, 1H), 8.38- 8.24 (m, 2H), 8.13- 8.01 (m, 2H), 7.66- 7.56 (m, 1H), 7.40- 7.30 (m, 2H), 7.23- 7.09 (m, 5H), 6.31- 6.05 (m, 2H), 5.01- 4.86 (m, 1H), 4.67- 4.59 (m, 1H), 4.43- 3.64 (m, 7H), 2.39- 2.26 (m, 1H), 2.23- 1.95 (m, 7H), 1.93- |
| | yl)carbamoyl) | | | 1.78 (m, 2H), 1.70- |
| | benzo[b]thio- | | | 1.58 (m, 1H), 1.55- |
| | phen-5- | | | 1.30 (m, 3H), 1.19- |
| | yl)methyl)(phe- | | | 1.11 (m, 2H), 0.97- |
| | noxy)phos- | | | 0.90 (m, 1H), 0.87- |
| | phoryl)-L- | | | 0.68 (m, 6H), 0.05- |
| | alaninate | | | −0.04 (m, 1H). |
| 374 | isobutyl ((fluoro(2- (((3S,6S,7aS, 8aR,9aR)-3- (3-(4- methylpyridin- 3- yl)azetidine- 1-carbonyl)- 5- oxodecahydro- 1H- cyclopropa[d] pyrrolo[1,2- a]azocin-6- | 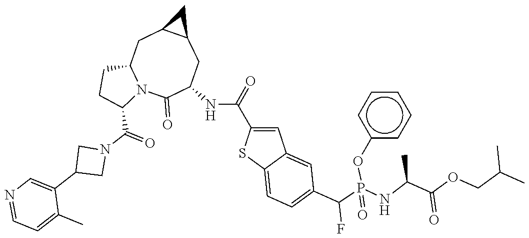 | 844.1 [M + H]⁺ | ¹H NMR (400 MHz, CD₃OD) δ 8.55-8.52 (m, 1H), 8.34-8.18 (m, 1H), 8.15-7.85 (m, 3H), 7.69-7.52 (m, 1H), 7.38-7.28 (m, 2H), 7.28-7.09 (m, 4H), 6.16-5.77 (m, 1H), 5.09-4.97 (m, 1H), 4.80-4.69 (m, 1H), 4.58-4.56 (m, 1H), 4.50-4.28 (m, 2H), 4.25-4.06 (m, 2H), 3.92-3.59 (m, 3H), 2.54-2.38 |
| | yl)carbamoyl) | | | (m, 1H), 2.37-2.11 |
| | benzo[b]thio- | | | (m, 7H), 2.05-1.69 |
| | phen-5- | | | (m, 4H), 1.47-1.37 |
| | yl)methyl)(phe- | | | (m, 1H), 1.35-1.21 |
| | noxy)phos- | | | (m, 2H), 1.11-0.77 |
| | phoryl)-L- | | | (m, 10H), 0.09-0.04 |
| | alaninate | | | (m, 1H). |
| 281 | isopropyl ((fluoro(2- (((3S,6S,7aS, 8aR,9aR)-3- (3-(4- methylpyridin- 3- yl)azetidine- 1-carbonyl)- 5- oxodecahydro- 1H- cyclopropa[d] pyrrolo[1,2- a]azocin-6- | 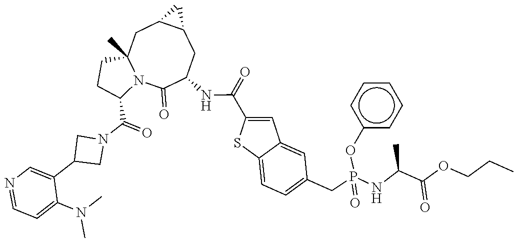 | 830.1 [M + H]⁺ | ¹H NMR (400 MHz, CD₃OD) δ 8.54-8.47 (m, 1H), 8.33-8.27 (m, 0.4H), 8.27-8.20 (m, 0.6H), 8.13-8.09 (m, 0.4H), 8.08-8.05 (m, 0.4H), 8.04-7.96 (m, 1.2H), 7.96-7.82 (m, 1H), 7.68-7.55 (m, 1H), 7.39-7.28 (m, 2H), 7.26-7.06 (m, 4H), 6.20-5.93 (m, 1H), 5.09-4.97 (m, 1.5H), 4.96-4.87 (m, 1H), 4.78-4.67 |
| | yl)carbamoyl) | | | (m, 1H), 4.61-4.54 |
| | benzo[b]thio- | | | (m, 1H), 4.50-4.44 |
| | phen-5- | | | (m, 0.5H), 4.42-4.25 |
| | yl)methyl)(phe- | | | (m, 2H), 4.23-4.05 |
| | noxy)phos- | | | (m, 2H), 3.93-3.69 |
| | phoryl)-L- | | | (m, 1H), 2.52-2.37 |
| | alaninate | | | (m, 1H), 2.36-2.08 |
| | | | | (m, 7H), 2.05-1.81 |
| | | | | (m, 2H), 1.80-1.67 |
| | | | | (m, 1H), 1.51-1.35 |
| | | | | (m, 1H), 1.27-1.07 |
| | | | | (m, 8H), 1.06-1.00 |
| | | | | (m, 1H), 0.99-0.78 |
| | | | | (m, 2H), 0.10-0.01 |
| | | | | (m, 1H). |
| 283 | propyl(2S)- 2-(((fluoro(2- (((3S,6S,7aS, 8aR,9aR)-3- (3-(4- methylpyridin- 3- yl)azetidine- 1-carbonyl)- 5- oxodecahydro- 1H- cyclopropa[d] pyrrolo[1,2- a]azocin-6- | 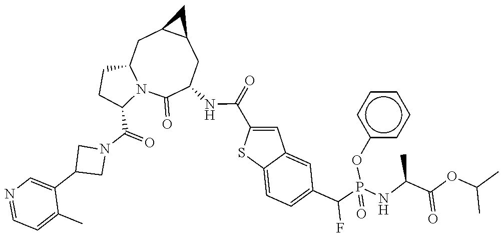 | 844.3 [M + H]⁺ | ¹H NMR (400 MHz, CD₃OD) δ 8.65-8.48 (m, 1H), 8.37-8.21 (m, 1H), 8.16-8.02 (m, 2H), 8.02-7.89 (m, 1H), 7.69-7.57 (m, 1H), 7.40-7.11 (m, 6H), 6.17-5.96 (m, 1H), 5.09-4.97 (m, 1.5H), 4.83-4.68 (m, 1H), 4.60-4.54 (m, 1H), 4.53-4.25 (m, 2.5H), 4.25-4.06 (m, 2H), 4.02-3.60 (m, 3H), 2.52-2.38 |
| | yl)carbamoyl) | | | (m, 1H), 2.36-2.10 |
| | benzo[b]thio- | | | (m, 7H), 2.06-1.89 |
| | phen-5- | | | (m, 2H), 1.81-1.69 |
| | yl)methyl)(phe- | | | (m, 1H), 1.68-1.37 |
| | noxy)phos- | | | (m, 5H), 1.01-0.62 |
| | phoryl)amino) | | | (m, 8H), 0.11-0.03 |
| | butanoate | | | (m, 1H). |
| 284 | propyl N- ((fluoro(2- (((3S,6S,7aS, 8aR,9aR)-3- (3-(4- methylpyridin- 3- yl)azetidine- 1-carbonyl)- 5- oxodecahydro- 1H- cyclopropa[d] pyrrolo[1,2- a]azocin-6- | 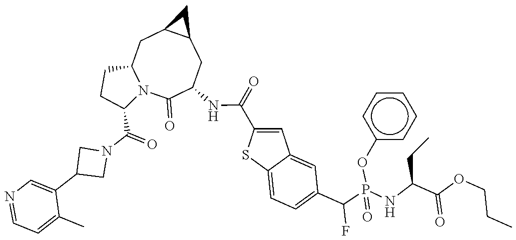 | 860.2 [M + H]⁺ | ¹H NMR (400 MHz, CD₃OD) δ 8.54 (d, J = 15.9 Hz, 1H), 8.33- 8.24 (m, 1H), 8.16- 8.02 (m, 2H), 8.01- 7.87 (m, 1H), 7.69- 7.58 (m, 1H), 7.38- 7.28 (m, 2H), 7.28- 7.12 (m, 4H), 6.22- 5.99 (m, 1H), 5.09- 4.97 (m, 1.5H), 4.81- 4.68 (m, 1H), 4.61- 4.53 (m, 1H), 4.52- 4.44 (m, 0.5H), 4.44- |
| | yl)carbamoyl) | | | 4.34 (m, 1H), 4.34- |
| | benzo[b]thio- | | | 4.26 (m, 1H), 4.24- |
| | phen-5- | | | 4.06 (m, 2H), 4.06- |
| | yl)methyl)(phe- | | | 3.77 (m, 3H), 3.60- |
| | noxy)phos- | | | 3.46 (m, 1H), 3.41- |
| | phoryl)-O- | | | 3.33 (m, 0.5H), 3.25 |
| | methyl-L- | | | (d, J = 5.6 Hz, 1H), |
| | serinate | | | 3.20-3.05 (m, 2.5H), |
| | | | | 2.53-2.37 (m, 1H), |
| | | | | 2.35-2.09 (m, 7H), |
| | | | | 2.05-1.86 (m, 2H), |
| | | | | 1.80-1.67 (m, 1H), |
| | | | | 1.64-1.37 (m, 3H), |
| | | | | 1.04-0.79 (m, 5H), |
| | | | | 0.12-0.01 (m, 1H). |
| 292 | propyl(((2- (((3S,6S,7aS, 8aR,9aR)-3- ((1R,5R)-2- acetyl-2,6- diaza- bicyclo[3.2.0] heptane-6- carbonyl)-5- oxodecahydro- 1H- cyclopropa[d] pyrrolo[1,2- a]azocin-6- yl)carbamoyl) | 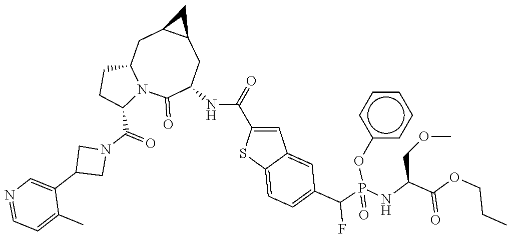 | 822.1 [M + H]⁺ | ¹H NMR (400 MHz, CD₃OD) δ 8.16-8.06 (m, 2H), 8.03-7.90 (m, 1H), 7.69-7.54 (m, 1H), 7.39-7.28 (m, 2H), 7.25-7.10 (m, 3H), 6.20-5.95 (m, 1H), 5.08-4.89 (m, 2H), 4.71-4.59 (m, 1H), 4.53-4.05 (m, 4H), 4.02-3.75 (m, 4H), 3.65-3.43 (m, 1H), 2.50-2.40 (m, 1H), 2.39-1.84 (m, 11H), 1.84-1.67 |
| | benzo[b]thio- | | | (m, 1H), 1.61-1.33 |
| | phen-5- | | | (m, 3H), 1.32-1.17 |
| | yl)fluorometh- | | | (m, 2H), 1.10-0.78 |
| | yl)(phenoxy) | | | (m, 6H), 0.08-0.05 |
| | phosphoryl)- | | | (m, 1H). |
| | L-alaninate | | | |
| 291 | propyl(((2- (((3S,6S,7aS, 8aR,9aR)-3- ((1S,5S)-2- acetyl-2,6- diaza- bicyclo[3.2.0] heptane- 6-carbonyl)- 5- oxodecahydro- 1H- cyclopropa[d] pyrrolo[1,2- a]azocin-6- yl)carbamoyl) | 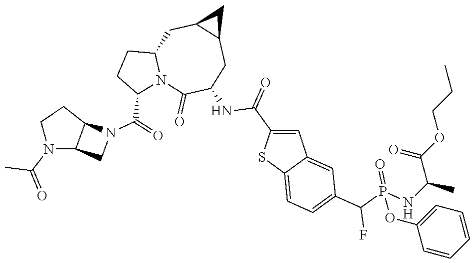 [SMILES: CCCOC(=O)[C@@H](C)NP(=O)(Oc1ccccc1)C(F)c1ccc2sc(C(=O)N[C@H]3C[C@@H]4C[C@@H]4C[C@H]4CC[C@@H](C(=O)N5C[C@@H]6[C@H]5CCN6C(C)=O)N4C3=O)cc2c1] | 822.1 [M + H]⁺ | ¹H NMR (400 MHz, CD₃OD) δ 8.12-8.08 (m, 2H), 8.02-7.90 (m, 1H), 7.66-7.62 (m, 1H), 7.38-7.27 (m, 2H), 7.25-7.11 (m, 3H), 6.20-5.97 (m, 1H), 5.28-4.92 (m, 2H), 4.83-4.55 (m, 2H), 4.45-4.12 (m, 2H), 4.02-3.72 (m, 5H), 3.69-3.40 (m, 1H), 2.52-2.38 (m, 1H), 2.36-1.83 (m, 11H), 1.80-1.68 (m, 1H), 1.64-1.35 |
| | benzo[b]thio- | | | (m, 3H), 1.26-1.17 |
| | phen-5- | | | (m, 2H), 1.08-0.79 |
| | yl)fluorometh- | | | (m, 6H), 0.08-0.05 |
| | yl)(phenoxy) | | | (m, 1H). |
| | phosphoryl)- | | | |
| | L-alaninate | | | |
| 232 | propyl(((2- (((3S,6S,8aR, 9aS,9bR)-8a- methyl-5- oxo-3-(3- (pyridin-3- yl)azetidine- 1- carbonyl)deca- hydro-1H- cyclopropa[c] pyrrolo[1,2- a]azocin-6- yl)carbamoyl) benzo[b]thio- phen-5- yl)methyl)(phe- | 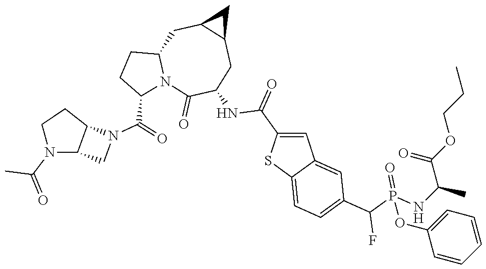 | 812.3 [M + H]⁺ | ¹H NMR (400 MHz, CD₃OD) δ 8.62-8.57 (m, 0.6H), 8.56-8.52 (m, 0.4H), 8.49-8.40 (m, 1H), 8.15-8.08 (m, 0.6H), 8.05-8.00 (m, 1H), 7.98-7.83 (m, 2.4H), 7.54-7.45 (m, 1.4H), 7.45-7.39 (m, 0.6H), 7.36-7.26 (m, 2H), 7.22-7.10 (m, 3H), 4.98 (t, J = 8.4 Hz, 0.4H), 4.74- 4.67 (m, 0.6H), 4.66- 4.59 (m, 1.6H), 4.59- 4.51 (m, 0.4H), 4.50- 4.38 (m, 1.6H), 4.33- |
| | noxy)phos- | | | 4.26 (m, 0.4H), 4.12- |
| | phoryl)-L- | | | 4.05 (m, 0.6H), 4.05- |
| | alaninate | | | 3.75 (m, 5.4H), 3.61- |
| | | | | 3.42 (m, 2H), 2.42- |
| | | | | 2.02 (m, 6H), 2.02- |
| | | | | 1.92 (m, 1H), 1.77- |
| | | | | 1.65 (m, 1H), 1.60- |
| | | | | 1.46 (m, 2H), 1.44- |
| | | | | 1.33 (m, 1H), 1.17- |
| | | | | 1.10 (m, 4H), 1.10- |
| | | | | 1.04 (m, 2H), 0.90- |
| | | | | 0.81 (m, 3H), 0.57- |
| | | | | 0.50 (m, 1H), 0.46- |
| | | | | 0.39 (m, 1H). |
| 233 | propyl(((2- (((3S,6S,7aS, 8aR,9aR)-8a- methyl-5- oxo-3-(3- (pyridin-3- yl)azetidine- 1- carbonyl)deca- hydro-1H- cyclopropa[d] pyrrolo[1,2- a]azocin-6- yl)carbamoyl) benzo[b]thio- | 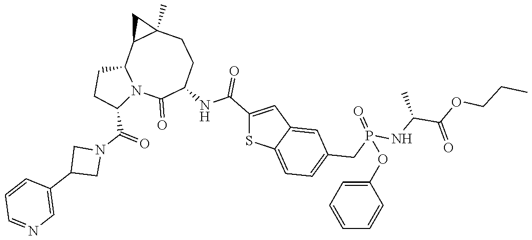 [SMILES: CCCOC(=O)[C@@H](C)NP(=O)(Cc1ccc2sc(C(=O)N[C@H]3CC[C@]4(C)C[C@@H]4[C@H]4CC[C@@H](C(=O)N5CC(c6cccnc6)C5)N4C3=O)cc2c1)Oc1ccccc1] | 812.2 [M + H]⁺ | ¹H NMR (400 MHz, CD₃OD) δ 8.56-8.35 (m, 2H), 8.12-8.02 (m, 2H), 7.96-7.85 (m, 2H), 7.51-7.44 (m, 1H), 7.38-7.26 (m, 3H), 7.20-7.14 (m, 3H), 5.04-4.90 (m, 1H), 4.71-4.62 (m, 1H), 4.54-4.37 (m, 2H), 4.30-4.25 (m, 1H), 4.06-3.78 (m, 5H), 3.59-3.43 (m, 2H), 2.58-2.49 (m, 1H), 2.29-1.93 |
| | phen-5- | | | (m, 7H), 1.59-1.48 |
| | yl)methyl)(phe- | | | (m, 2H), 1.31 (s, 3H), |
| | noxy)phos- | | | 1.26-1.03 (m, 4H), |
| | phoryl)-L- | | | 1.01-0.93 (m, 1H), |
| | alaninate | | | 0.89-0.83 (m, 3H), |
| | | | | 0.59-0.56 (m, 1H), |
| | | | | 0.36-0.33 (m, 1H). |
| 240 | propyl(((2- (((3S,6S,7aR, 8aS,9aR)-8a- methyl-5- oxo-3-(3- (pyridin-3- yl)azetidine- 1- carbonyl)deca- hydro-1H- cyclopropa[d] pyrrolo[1,2- a]azocin-6- yl)carbamoyl) | 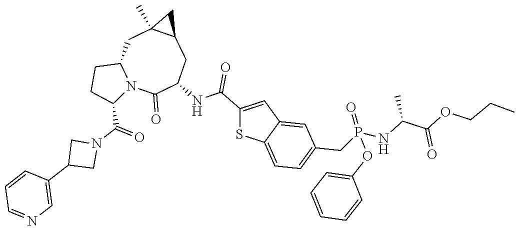 [SMILES: CCCOC(=O)[C@@H](C)NP(=O)(Cc1ccc2sc(C(=O)N[C@H]3C[C@@H]4C[C@]4(C)C[C@H]4CC[C@@H](C(=O)N5CC(c6cccnc6)C5)N4C3=O)cc2c1)Oc1ccccc1] | 812.2 [M + H]⁺ | ¹H NMR (400 MHz, CD₃OD) δ 8.61-8.38 (m, 2H), 8.11-8.00 (m, 2H), 7.97-7.85 (m, 2H), 7.54-7.39 (m, 2H), 7.37-7.27 (m, 2H), 7.22-7.10 (m, 3H), 5.01-4.89 (m, 1H), 4.74-4.58 (m, 1H), 4.54-4.28 (m, 3H), 4.08-3.77 (m, 5H), 3.62-3.44 (m, 2H), 2.42-2.14 (m, 4H), 1.99-1.64 |
| | benzo[b]thio- | | | (m, 4H), 1.59-1.47 |
| | phen-5- | | | (m, 2H), 1.32-1.20 |
| | yl)methyl)(phe- | | | (m, 4H), 1.18-0.95 |
| | noxy)phos- | | | (m, 4H), 0.89-0.84 |
| | phoryl)-L- | | | (m, 3H), 0.67-0.64 |
| | alaninate | | | (m, 1H), 0.36-0.34 |
| | | | | (m, 1H). |
| 436 | isopropyl ((fluoro(2- (((3S,6S,7aS, 8aR,9aR)-3- (3-(4- methylpyridin- 3- yl)azetidine- 1-carbonyl)- 5- oxodecahydro- 1H- cyclopropa[d] pyrrolo[1,2- a]azocin-6- yl)carbamoyl) | 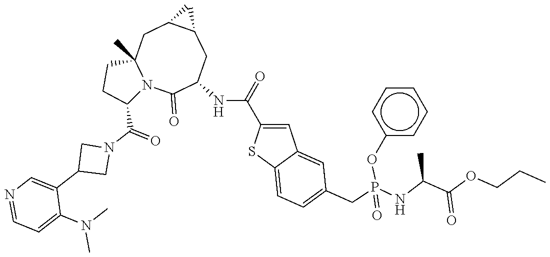 | 855.2 [M + H]⁺ | ¹H NMR (400 MHz, CD₃OD) δ 8.54-8.47 (m, 1H), 8.33-8.27 (m, 0.4H), 8.27-8.20 (m, 0.6H), 8.13-8.09 (m, 0.4H), 8.08-8.05 (m, 0.4H), 8.04-7.96 (m, 1.2H), 7.96-7.82 (m, 1H), 7.68-7.55 (m, 1H), 7.39-7.28 (m, 2H), 7.26-7.06 (m, 4H), 6.20-5.93 (m, 1H), 5.09-4.97 (m, 1.5H), 4.96-4.87 (m, 1H), 4.78-4.67 (m, 1H), 4.61-4.54 |
| | benzo[b]thio- | | | (m, 1H), 4.50-4.44 |
| | phen-5- | | | (m, 0.5H), 4.42-4.25 |
| | yl)methyl)(phe- | | | (m, 2H), 4.23-4.05 |
| | noxy)phos- | | | (m, 2H), 3.93-3.69 |
| | phoryl)-L- | | | (m, 1H), 2.52-2.37 |
| | alaninate | | | (m, 1H), 2.36-2.08 |
| | | | | (m, 7H), 2.05-1.81 |
| | | | | (m, 2H), 1.80-1.67 |
| | | | | (m, 1H), 1.51-1.35 |
| | | | | (m, 1H), 1.27-1.07 |
| | | | | (m, 8H), 1.06-1.00 |
| | | | | (m, 1H), 0.99-0.78 |
| | | | | (m, 2H), 0.10-0.01 |
| | | | | (m, 1H). |

The following compounds in Table 36 were prepared according to the representative procedure described above for the synthesis of (difluoro(2-(((3S,6S,7aS,8aR,9aR)-3-(3-((R)-2-methylmorpholine-4-carbonyl)azetidine-1-carbonyl)-5-oxodecahydro-1H-cyclopropa[d]pyrrolo[1,2-a]azocin-6-yl)carbamoyl)benzo[b]thiophen-5-yl)methyl)phosphonic acid (Example 1) utilizing the appropriate starting materials and modifications.

TABLE 36

| Com- | | | LCMS | |
|---|---|---|---|---|
| pound | Name | Structure | [M + H]⁺ | NMR |
| 400 | ((2- (((3S,6S,7aS, 8aR,9aR)-3- (3-(4- bromo- isoxazol-3- yl)azetidine- 1-carbonyl)- 5-oxo- decahydro- 1H-cyclo- propa[d] pyrrolo[1,2- a]azocin-6- yl)carbamoyl) benzo[b] thiophen-5- yl)fluoro- methyl) | 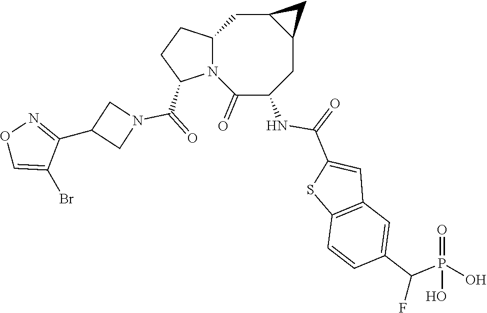 [SMILES: O=C(N[C@H]1C[C@@H]2C[C@@H]2C[C@H]2CC[C@@H](C(=O)N3CC(c4nocc4Br)C3)N2C1=O)c1cc2cc(C(F)P(=O)(O)O)ccc2s1] | 695.1 | ¹H NMR (400 MHz, CD₃OD) δ 8.88-8.79 (m, 1H), 8.08 (d, J = 15.6 Hz, 1H), 8.03 (d, J = 8.8 Hz, 1H), 7.86 (t, J = 8.0 Hz, 1H), 7.67-7.59 (m, 1H), 5.76-5.53 (m, 1H), 5.05 (s, 1H), 4.80 (d, J = 8.0 Hz, 1H), 4.70 (t, J = 9.2 Hz, 1H), 4.56-4.50 (m, 1H), 4.49-4.41 (m, 1H), 4.37-4.32 (m, 1H), 4.32-4.24 (m, 1H), 4.12-4.02 (m, 1H), 2.52-2.38 (m, 1H), 2.35-2.07 (m, 4H), 2.03-1.89 (m, 2H), |
| | phosphonic | | | 1.81-1.69 (m, 1H), |
| | acid | | | 1.48-1.36 (m, 1H), |
| | | | | 1.01-0.90 (m, 1H), |
| | | | | 0.88-0.79 (m, 1H), |
| | | | | 0.10-0.02 (m, 1H) |
| 394 | ((S)-(2- (((3S,6S,7aR, 8aS,9aR)-3- (3-(4- (dimethyl- amino) pyridin-3- yl)azetidine- 1-carbonyl)- 5-oxodeca- hydro-1H- cyclopropa[d] pyrrolo[1,2- a]azocin-6- yl)carbamoyl) | 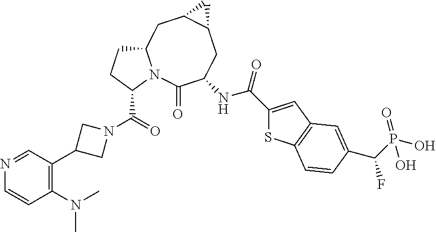 [SMILES: CN(C)c1ccncc1C1CN(C(=O)[C@@H]2CC[C@@H]3C[C@@H]4C[C@@H]4C[C@H](NC(=O)c4cc5cc([C@@H](F)P(=O)(O)O)ccc5s4)C(=O)N32)C1] | 670.3 | ¹H NMR (400 MHz, CD₃OD) δ 8.24 (d, J = 18.4 Hz, 1H), 8.08- 7.83 (m, 4H), 7.59- 7.53 (m, 1H), 7.06- 6.88 (m, 1H), 5.87- 5.72 (m, 1H), 4.94- 4.91 (m, 1H), 4.70 (t, J = 8.8 Hz, 1H), 4.50- 4.24 (m, 5H), 4.05- 3.99 (m, 1H), 3.15- 3.02 (m, 6H), 2.42- 2.14 (m, 4H), 2.06- 1.84 (m, 3H), 1.72- 1.59 (m, 1H), 1.47- |
| | benzo[b] | | | 1.24 (m, 2H), 0.89- |
| | thiophen-5- | | | 0.84 (m, 1H), 0.20- |
| | yl) | | | 0.16 (m, 1H) |
| | fluoromethyl) | | | |
| | phosphonic | | | |
| | acid | | | |
| 393 | ((2- (((3S,6S,7aR, 8aS,9aR)-3- (3-(4- (dimethyl- amino) pyridin-3- yl)azetidine- 1-carbonyl)- 5-oxodeca- hydro-1H- cyclopropa[d] pyrrolo[1,2- a]azocin-6- yl)carbamoyl) | 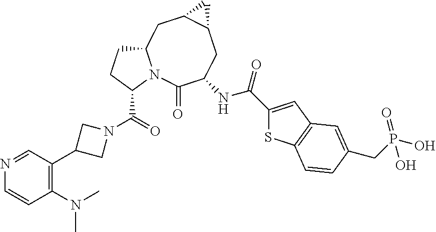 [SMILES: CN(C)c1ccncc1C1CN(C(=O)[C@@H]2CC[C@@H]3C[C@@H]4C[C@@H]4C[C@H](NC(=O)c4cc5cc(CP(=O)(O)O)ccc5s4)C(=O)N32)C1] | 652.2 | ¹H NMR (400 MHz, CD₃OD) δ 8.35-8.26 (m, 1H), 8.13-7.98 (m, 2H), 7.85-7.77 (m, 2H), 7.44-7.39 (m, 1H), 7.12-7.02 (m, 1H), 4.93 (d, J = 2.8 Hz, 1H), 4.70 (t, J = 8.4 Hz, 1H), 4.54- 4.47 (m, 1H), 4.44- 4.38 (m, 2H), 4.36- 4.28 (m, 2H), 4.07- 4.01 (m, 1H), 3.27- 3.18 (m, 2H), 3.13- 3.12 (m, 6H), 2.44- |
| | benzo[b] | | | 2.34 (m, 1H), 2.32- |
| | thiophen-5- | | | 2.13 (m, 3H), 2.04- |
| | yl)methyl) | | | 1.86 (m, 3H), 1.68- |
| | phosphonic | | | 1.65 (m, 1H), 1.45- |
| | acid | | | 1.29 (m, 2H), 0.89- |
| | | | | 0.84 (m, 1H), 0.19- |
| | | | | 0.16 (m, 1H) |
| 391 | ((S)- fluoro(2- (((3S,6S,7aR, 8aS,9aR)-3- (3-(4- morpholino- pyridin-3- yl)azetidine- 1-carbonyl)- 5-oxodeca- hydro-1H- cyclopropa[d] pyrrolo[1,2- a]azocin-6- yl)carbamoyl) | 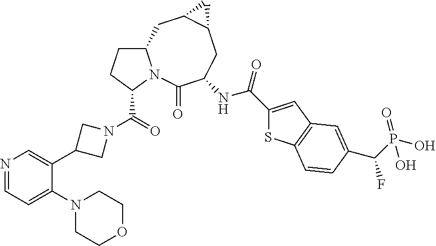 [SMILES: O=C(N[C@H]1C[C@H]2C[C@H]2C[C@H]2CC[C@@H](C(=O)N3CC(c4cnccc4N4CCOCC4)C3)N2C1=O)c1cc2cc([C@@H](F)P(=O)(O)O)ccc2s1] | 712.2 | ¹H NMR (400 MHz, DMSO-d₆) δ 8.77 (dd, J = 7.2, 11.2 Hz, 1H), 8.41-8.36 (m, 1H), 8.24 (d, J = 19.2 Hz, 1H), 8.07-8.00 (m, 1H), 7.97 (d, J = 10.0 Hz, 1H), 7.58- 7.48 (m, 1H), 7.29- 7.10 (m, 1H), 5.92- 5.70 (m, 1H), 4.84- 4.79 (m, 1H), 4.77- 4.61 (m, 1H), 4.42- 4.34 (m, 1H), 4.33- 4.26 (m, 2H), 4.25- |
| | benzo[b] | | | 4.16 (m, 2H), 3.95 |
| | thiophen-5- | | | (dd, J = 6.0, 9.2 Hz, |
| | yl)methyl) | | | 1H), 3.72 (s, 4H), |
| | phosphonic | | | 3.20 (s, 4H), 2.27- |
| | acid | | | 2.16 (m, 2H), 2.09- |
| | | | | 1.99 (m, 2H), 1.94- |
| | | | | 1.84 (m, 2H), 1.74- |
| | | | | 1.62 (m, 1H), 1.56- |
| | | | | 1.42 (m, 1H), 1.36- |
| | | | | 1.27 (m, 1H), 1.25- |
| | | | | 1.19 (m, 1H), 0.81- |
| | | | | 0.72 (m, 1H), 0.08- |
| | | | | 0.01 (m, 1H) |
| 390 | (fluoro(2- (((3S,6S,7aS, 8aR,9aR)-5- oxo-3-(3- phenyl- azetidine-1- carbonyl) decahydro-1H- cyclopropa[d] pyrrolo[1,2- a]azocin-6- yl)carbamoyl) benzo[b] thiophen-5- yl)methyl) phosphonic acid | 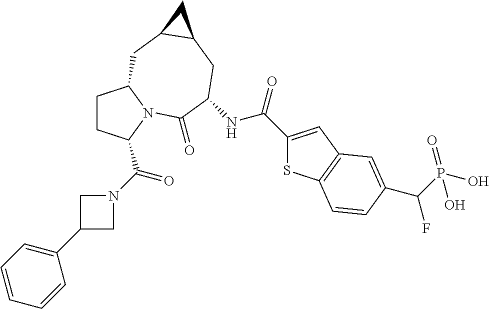 [SMILES: O=C(N[C@H]1C[C@@H]2C[C@@H]2C[C@H]2CC[C@@H](C(=O)N3CC(c4ccccc4)C3)N2C1=O)c1cc2cc(C(F)P(=O)(O)O)ccc2s1] | 626 | ¹H NMR (400 MHz, CD₃OD) δ 8.15-7.88 (m, 3H), 7.58 (d, J = 8.0 Hz, 1H), 7.44- 7.19 (m, 5H), 5.92- 5.74 (m, 1H), 5.10- 4.93 (m, 2H), 4.66 (d, J = 7.6 Hz, 1H), 4.60- 4.53 (m, 1H), 4.38- 4.23 (m, 2H), 4.03- 3.86 (m, 2H), 2.52- 2.41 (m, 1H), 2.37- 2.12 (m, 3H), 2.05- 1.89 (m, 2H), 1.79- 1.68 (m, 1H), 1.51- 1.40 (m, 1H), 1.39- 1.35 (m, 1H), 1.05- 0.92 (m, 1H), 0.90- 0.79 (m, 1H), 0.12- |
| | | | | 0.01 (m, 1H) |
| 389 | (fluoro(2- (((3S,6S,7aS, 8aR,9aR)-5- oxo-3-(1- azaspiro[3.4] octane-1- carbonyl) decahydro-1H- cyclopropa[d] pyrrolo[1,2- a]azocin-6- yl)carbamoyl) benzo[b] thiophen-5- yl)methyl) phosphonic acid | 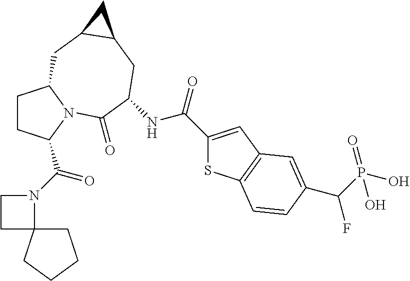 [SMILES: O=C(N[C@H]1C[C@@H]2C[C@@H]2C[C@H]2CC[C@@H](C(=O)N3CCC34CCCC4)N2C1=O)c1cc2cc(C(F)P(=O)(O)O)ccc2s1] | 604.1 | ¹H NMR (400 MHz, CD₃OD) δ 8.09 (s, 1H), 8.02 (s, 1H), 7.922-7.901 (m, 1H), 7.603-7.585 (m, 1H), 5.86-5.69 (m, 1H), 5.041-4.998 (m, 1H), 4.72-4.66 (m, 1H), 4.34-4.26 (m, 1H), 3.632-3.590 (m, 2H), 2.56-2.45 (m, 1H), 2.38-1.97 (m, 10H), 1.95-1.67 (m, 8H), 1.02-0.95 (m, 1H), 0.95-0.89 (m, 1H), 0.206-0.171 (m, 1H) |
| 388 | (fluoro(2- (((3S,6S,7aS, 8aR,9aR)-5- oxo-3-(1- azaspiro[3.5] nonane-1- carbonyl) decahydro-1H- cyclopropa[d] pyrrolo[1,2- a]azocin-6- yl)carbamoyl) benzo[b] thiophen-5- yl)methyl) phosphonic acid | 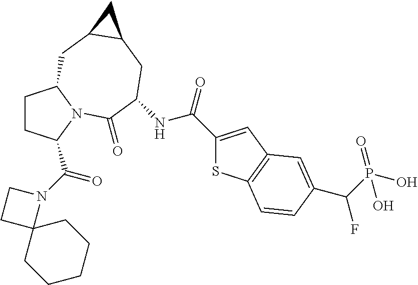 [SMILES: O=C(N[C@H]1C[C@@H]2C[C@@H]2C[C@H]2CC[C@@H](C(=O)N3CCC34CCCCC4)N2C1=O)c1cc2cc(C(F)P(=O)(O)O)ccc2s1] | 618.3 | ¹H NMR (400 MHz, CD₃OD) δ 8.09 (s, 1H), 8.03 (s, 1H), 7.91 (d, J = 8.4 Hz, 1H), 7.60 (d, J = 8.0 Hz, 1H), 5.89-5.66 (m, 1H), 5.02 (t, J = 8.8 Hz, 1H), 4.76- 4.65 (m, 1H), 4.39- 4.26 (m, 1H), 3.60 (d, J = 3.2 Hz, 2H), 2.52- 2.47 (m, 1H), 2.42- 2.25 (m, 3H), 2.16 (s, 4H), 2.08-1.98 (m,1H), 1.92-1.74 (m, 4H), 1.71-1.42 (m, 7H), 1.12-1.01 (m, 1H), 1.00-0.89 |
| | | | | (m, 2H), 0.19 (d, J = |
| | | | | 4.4 Hz, 1H) |
| 387 | (fluoro(2- (((3S,6S,7aS, 8aR,9aR)-3- (4-(oxetan-3- yl)-1,2,3,4- tetrahydro- quinoxaline-1- carbonyl)-5- oxodecahydro- 1H- cyclopropa[d] pyrrolo[1,2- a]azocin-6- yl)carbamoyl) benzo[b] thiophen-5- yl)methyl) phosphonic acid | 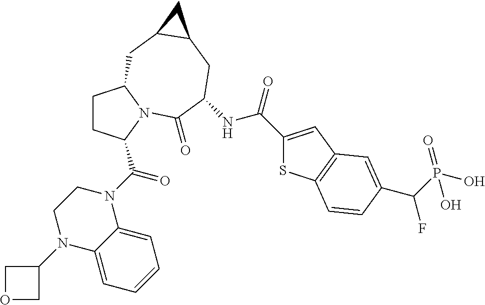 [SMILES: O=C(N[C@H]1C[C@@H]2C[C@@H]2C[C@H]2CC[C@@H](C(=O)N3CCN(C4COC4)c4ccccc43)N2C1=O)c1cc2cc(C(F)P(=O)(O)O)ccc2s1] | 683.4 | ¹H NMR (400 MHz, CD₃OD) δ 7.98-7.84 (m, 2H), 7.84-7.73 (m, 1H), 7.60 (d, J = 7.6 Hz, 1H), 7.41- 7.26 (m, 1H), 7.03 (t, J = 7.6 Hz, 1H), 6.74 (t, J = 7.2 Hz, 1H), 6.38 (d, J = 8.0 Hz, 1H), 5.76-5.57 (m, 1H), 5.18 (d, J = 8.4 Hz, 1H), 5.04-4.95 (m, 1H), 4.89 (d, J = 5.6 Hz, 2H), 4.83 (s, 1H), 4.76 (s, 2H), 4.26 (d, J = 2.8 Hz, 1H), 4.01-3.65 (m, 2H), 3.60-3.51 (m, 1H), 3.49-3.40 (m, |
| | | | | 1H), 2.54-2.43 (m, |
| | | | | 1H), 2.38-2.31 (m, |
| | | | | 1H), 2.31-2.22 (m, |
| | | | | 1H), 2.21-2.04 (m, |
| | | | | 2H), 2.03-1.90 (m, |
| | | | | 2H), 1.81-1.69 (m, |
| | | | | 1H), 1.64-1.45 (m, |
| | | | | 1H), 1.06-0.96 (m, |
| | | | | 1H), 0.92-0.84 (m, |
| | | | | 1H), 0.12-0.04 (m, |
| | | | | 1H) |
| 386 | ((2- (((3S,6S,7aS, 8aR,9aR)-3- (2,3-dihydro- 1H- pyrido[2,3- b][1,4]oxazine- 1- carbonyl)-5- oxodecahydro- 1H- cyclopropa[d] pyrrolo[1,2- a]azocin-6- yl)carbamoyl) benzo[b] thiophen-5-yl) | 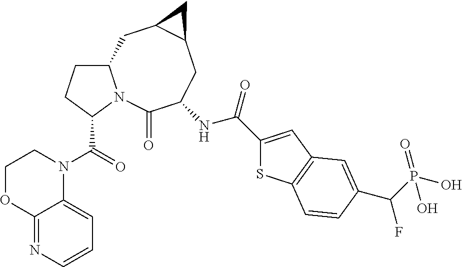 [SMILES: O=C(N[C@H]1C[C@@H]2C[C@@H]2C[C@H]2CC[C@@H](C(=O)N3CCOc4ncccc43)N2C1=O)c1cc2cc(C(F)P(=O)(O)O)ccc2s1] | 629.4 | ¹H NMR (400 MHz, DMSO-d₆) δ 8.85 (d, J = 7.6 Hz, 1H), 8.26 (d, J = 1.6 Hz, 1H), 8.02 (d, J = 8.4 Hz, 1H), 7.97 (s, 1H), 7.92 (s, 1H), 7.52 (d, J = 8.4 Hz, 1H), 6.99 (dd, J = 4.8, 7.6 Hz, 1H), 5.92-5.73 (m, 1H), 5.00-4.90 (m, 2H), 4.46-4.37 (m, 2H), 4.26-4.15 (m, 2H), 4.05-3.98 (m, 1H), 2.39-2.32 (m, 1H), 2.23-2.09 (m, 4H), 2.01 (s, 1H), |
| | fluoromethyl) | | | 1.90-1.82 (m, 1H), |
| | phosphonic | | | 1.74-1.65 (m, 1H), |
| | acid | | | 1.42-1.36 (m, 1H), |
| | | | | 0.87-0.74 (m, 2H), |
| | | | | 0.09-−0.03 (m, 1H) |
| 385 | ((2- (((3S,6S,7aS, 8aR,9aR)-3- (3,4-dihydro- 2H- pyrido[3,2- b][1,4]oxazine- 4- carbonyl)-5- oxodecahydro- 1H- cyclopropa[d] pyrrolo[1,2- a]azocin-6- yl)carbamoyl) benzo[b] thiophen-5- yl)fluoro- | 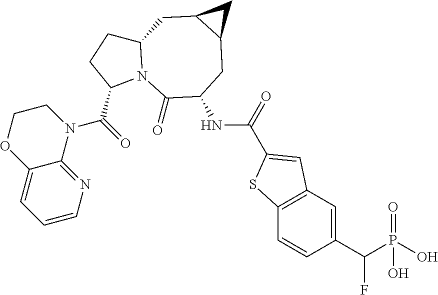 [SMILES: O=C(N[C@H]1C[C@@H]2C[C@@H]2C[C@H]2CC[C@@H](C(=O)N3CCOc4cccnc43)N2C1=O)c1cc2cc(C(F)P(=O)(O)O)ccc2s1] | 629.2 | ¹H NMR (400 MHz, DMSO-d₆) δ 8.78 (d, J = 7.6 Hz, 1H), 8.22 (d, J = 1.6 Hz, 1H), 8.01 (d, J = 8.4 Hz, 1H), 7.96-7.94 (m, 1H), 7.51 (d, J = 8.4 Hz, 1H), 7.34 (dd, J = 1.6, 8.0 Hz, 1H), 7.12 (dd, J = 4.8, 8.0 Hz, 1H), 5.89-5.75 (m, 1H), 5.32 (dd, J = 2.4, 8.8 Hz, 1H), 4.92- 4.86 (m, 1H), 4.53- 4.47 (m, 1H), 4.42- 4.38 (m, 1H), 4.21 (d, J = 6.4 Hz, 1H), 4.08- 4.02 (m, 1H), 3.38- |
| | methyl) | | | 3.31 (m, 1H), 2.39- |
| | phosphonic | | | 2.27 (m, 2H), 2.23- |
| | acid | | | 2.19 (m, 1H), 2.15- |
| | | | | 2.03 (m, 3H), 1.89- |
| | | | | 1.81 (m, 1H), 1.73- |
| | | | | 1.62 (m, 1H), 1.46- |
| | | | | 1.37 (m, 1H), 0.87- |
| | | | | 0.82 (m, 1H), 0.79- |
| | | | | 0.75 (m, 1H), 0.05- |
| | | | | 0.01 (m, 1H) |
| 384 | (fluoro(2- (((3S,6S,7aS, 8aR,9aR)-3- (7-fluoro- 3,4-dihydro- 2H- benzo[b][1,4] oxazine-4- carbonyl)-5- oxodecahydro- 1H- cyclopropa[d] pyrrolo[1,2- a]azocin-6- yl)carbamoyl) benzo[b] thiophen-5- yl)methyl) | 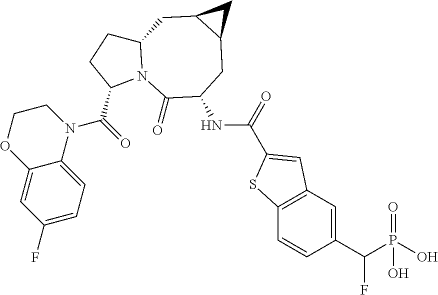 [SMILES: O=C(N[C@H]1C[C@@H]2C[C@@H]2C[C@H]2CC[C@@H](C(=O)N3CCOc4cc(F)ccc43)N2C1=O)c1cc2cc(C(F)P(=O)(O)O)ccc2s1] | 646.1 | ¹H NMR (400 MHz, CD₃OD) δ 8.04-7.85 (m, 3H), 7.57 (d, J = 8.4 Hz, 2H), 6.62- 6.59 (m, 2H), 5.88- 5.75 (m, 1H), 5.12- 5.03 (m, 2H), 4.35- 4.30 (m, 4H), 3.51- 3.46 (m, 1H), 2.48- 2.22 (m, 5H), 2.09 (d, J = 8.8 Hz, 1H), 2.00- 1.92 (m, 1H), 1.80- 1.75 (m, 1H), 1.53- 1.47 (m, 1H), 1.00- 0.95 (m, 1H), 0.89- 0.85 (m, 1H), 0.07- 0.05 (m, 1H) |
| | phosphonic | | | |
| | acid | | | |
| 383 | (fluoro(2- (((3S,6S,7aS, 8aR,9aR)-3- (7-methyl- 3,4-dihydro- 2H- benzo[b][1,4] oxazine-4- carbonyl)-5- oxodecahydro- 1H- cyclopropa[d] pyrrolo[1,2- a]azocin-6- yl)carbamoyl) benzo[b] thiophen-5- yl)methyl) phosphonic acid | 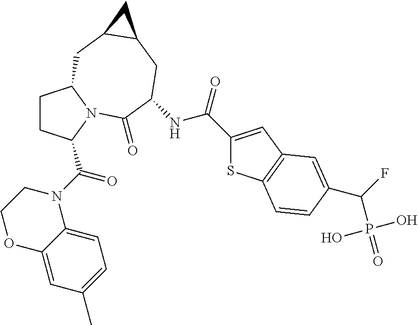 [SMILES: Cc1ccc2c(c1)OCCN2C(=O)[C@@H]1CC[C@@H]2C[C@H]3C[C@H]3C[C@H](NC(=O)c3cc4cc(C(F)P(=O)(O)O)ccc4s3)C(=O)N21] | 642.1 | ¹H NMR (400 MHz, DMSO-d₆) δ 8.85- 8.78 (m, 1H), 8.24 (s, 1H), 8.00-7.88 (m, 2H), 7.51 (d, J = 8.0 Hz, 1H), 6.68 (s, 2H), 5.80-5.67 (m, 1H), 4.94-4.93 (m, 2H), 4.26-4.18 (m, 4H), 3.81-3.72 (m, 1H), 2.35-2.32 (m, 1H), 2.12 (s, 2H), 2.00- 1.98 (m, 1H), 1.86- 1.82 (m, 1H), 1.72- 1.63 (m, 1H), 1.53- 1.40 (m, 2H), 1.23 (s, 3H), 0.85-0.75 (m, 3H), 0.02 (d, J = 4.4 Hz, 1H) |
| 380 | ((2- (((3S,6S,7aS, 8aR,9aR)-5- oxo-3-(3- (pyridin-3- ylmethyl) azetidine-1- carbonyl) decahydro-1H- cyclopropa[d] pyrrolo[1,2- a]azocin-6- yl)carbamoyl) benzo[b] | 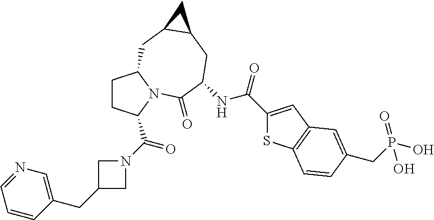 [SMILES: O=C(N[C@H]1C[C@@H]2C[C@@H]2C[C@H]2CC[C@@H](C(=O)N3CC(Cc4cccnc4)C3)N2C1=O)c1cc2cc(CP(=O)(O)O)ccc2s1] | 623.1 | ¹H NMR (400 MHz, CD₃OD) δ 8.57-8.46 (m, 1H), 8.28 (d, J = 6.4 Hz, 1H), 8.12 (d, J = 8.0 Hz, 1H), 7.96- 7.91 (m, 1H), 7.79- 7.58 (m, 3H), 7.39- 7.32 (m, 1H), 5.09- 5.04 (m, 1H), 4.67- 4.44 (m, 2H), 4.37- 4.26 (m, 2H), 4.18- 3.98 (m, 2H), 3.76- 3.67 (m, 1H), 3.25- 3.19 (m, 2H), 3.11 (d, |
| | thiophen-5- | | | J = 8.0 Hz, 1H), 3.00 |
| | yl)methyl) | | | (s, 1H), 2.56-2.41 |
| | phosphonic | | | (m, 1H), 2.35-2.12 |
| | acid | | | (m, 4H), 2.03-1.92 |
| | | | | (m, 2H), 1.83-1.69 |
| | | | | (m, 1H), 1.46-1.36 |
| | | | | (m, 1H), 1.00-0.90 |
| | | | | (m, 1H), 0.88-0.80 |
| | | | | (m, 1H), 0.11-0.06 |
| | | | | (m, 1H) |
| 379 | ((2- (((3S,6S,7aS, 8aR,9aR)-3- (3-((1- methyl-1H- pyrazol-4- yl)methyl) azetidine-1- carbonyl)-5- oxodecahydro- 1H- | 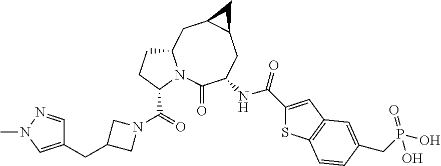 [SMILES: Cn1cc(CC2CN(C(=O)[C@@H]3CC[C@@H]4C[C@H]5C[C@H]5C[C@H](NC(=O)c5cc6cc(CP(=O)(O)O)ccc6s5)C(=O)N43)C2)cn1] | 626.4 | ¹H NMR (400 MHz, CD₃OD) δ 8.11-7.98 (m, 1H), 7.93-7.73 (m, 2H), 7.56-7.29 (m, 3H), 5.06-4.99 (m, 2H), 4.62-4.45 (m, 1H), 4.34-4.18 (m, 2H), 4.11-3.96 (m, 1H), 3.93-3.72 (m, 3H), 3.70-3.57 (m, 1H), 3.29-3.21 |
| | cyclopropa[d] | | | (m, 2H), 2.91-2.62 |
| | pyrrolo[1,2- | | | (m, 3H), 2.51-2.38 |
| | a]azocin-6- | | | (m, 1H), 2.34-2.04 |
| | yl)carbamoyl) | | | (m, 4H), 2.01-1.82 |
| | benzo[b] | | | (m, 2H), 1.78-1.63 |
| | thiophen-5- | | | (m, 1H), 1.47-1.33 |
| | yl)methyl) | | | (m, 1H), 0.99-0.77 |
| | phosphonic | | | (m, 2H), 0.09-−0.04 |
| | acid | | | (m, 1H) |
| 378 | ((2- (((3S,6S,7aS, 8aR,9aR)-3- (3-(2,5- difluoro- benzyl) azetidine- 1-carbonyl)- 5-oxodeca- hydro-1H- cyclopropa[d] pyrrolo[1,2- a]azocin-6- | 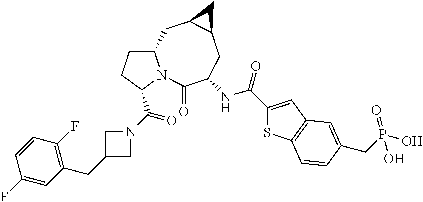 [SMILES: O=C(N[C@H]1C[C@@H]2C[C@@H]2C[C@H]2CC[C@@H](C(=O)N3CC(Cc4cc(F)ccc4F)C3)N2C1=O)c1cc2cc(CP(=O)(O)O)ccc2s1] | 658.2 | ¹H NMR (400 MHz, CD₃OD) δ 8.07- 8.01 (m, 1H), 7.89- 7.80 (m, 2H), 7.45- 7.41 (m, 1H), 7.11- 7.01 (m, 2H), 7.00- 6.91 (m, 1H), 5.06- 4.98 (m, 1H), 4.69- 4.58 (m, 1H), 4.52- 4.45 (m, 1H), 4.36- 4.23 (m, 2H), 4.14- 3.92 (m, 2H), 3.77- 3.63 (m, 1H), 3.29- |
| | yl)carbamoyl) | | | 3.21 (m, 2H), 3.03- |
| | benzo[b] | | | 3.02 (m, 1H), 3.03- |
| | thiophen-5- | | | 2.98 (m, 1H), 2.51- |
| | yl)methyl) | | | 2.39 (m, 1H), 2.33- |
| | phosphonic | | | 2.08 (m, 4H), 2.02- |
| | acid | | | 1.88 (m, 2H), 1.79- |
| | | | | 1.67 (m, 1H), 1.48- |
| | | | | 1.36 (m, 1H), 1.01- |
| | | | | 0.80 (m, 2H), 0.08- |
| | | | | 0.03 (m, 1H) |
| 377 | ((2- (((3S,6S,7aS, 8aR,9aR)-3- (3-(2,4- difluoro- benzyl) azetidine- 1-carbonyl)- 5-oxodeca- hydro-1H- cyclopropa[d] pyrrolo[1,2- | 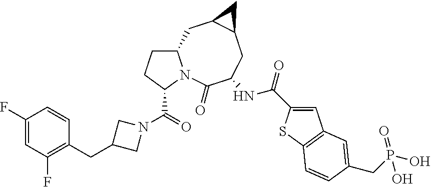 [SMILES: O=C(N[C@H]1C[C@@H]2C[C@@H]2C[C@H]2CC[C@@H](C(=O)N3CC(Cc4ccc(F)cc4F)C3)N2C1=O)c1cc2cc(CP(=O)(O)O)ccc2s1] | 658.2 | ¹H NMR (400 MHz, CD₃OD) δ 8.05 (d, J = 11.2 Hz, 1H), 7.90- 7.77 (m, 2H), 7.43 (d, J = 7.6 Hz, 1H), 7.36- 7.24 (m, 1H), 7.08- 6.74 (m, 2H), 5.07- 4.97 (m, 2H), 4.76- 4.56 (m, 1H), 4.53- 4.40 (m, 1H), 4.34- 4.22 (m, 2H), 4.09- 3.94 (m, 1H), 3.77- |
| | a]azocin-6- | | | 3.61 (m, 1H), 3.28 (s, |
| | yl)carbamoyl) | | | 1H), 3.25-3.20 (m, |
| | benzo[b] | | | 1H), 2.97 (s, 2H), |
| | thiophen-5- | | | 2.53-2.38 (m, 1H), |
| | yl)methyl) | | | 2.36-2.07 (m, 4H), |
| | phosphonic | | | 2.05-1.83 (m, 2H), |
| | acid | | | 1.80-1.66 (m, 1H), |
| | | | | 1.46-1.30 (m, 1H), |
| | | | | 1.03-0.73 (m, 2H), |
| | | | | 0.14-−0.04 (m, 1H) |
| 376 | ((2- (((3S,6S,7aS, 8aR,9aR)-3- (((3S,4S)-1- (1,5- dimethyl-1H- imidazol-2- yl)-4- fluoro- pyrrolidin-3- yl)carbamoyl)- 5- oxodecahydro- 1H- cyclopropa[d] pyrrolo[1,2- a]azocin-6- yl)carbamoyl) benzo[b] thiophen-5-yl) fluoromethyl) | 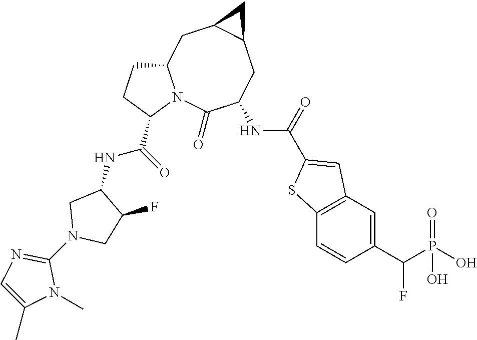 [SMILES: Cc1cnc(N2C[C@H](NC(=O)[C@@H]3CC[C@@H]4C[C@H]5C[C@H]5C[C@H](NC(=O)c5cc6cc(C(F)P(=O)(O)O)ccc6s5)C(=O)N43)[C@@H](F)C2)n1C] | 691.1 | ¹H NMR (400 MHz, DMSO-d₆) δ 8.77 (d, J = 6.8 Hz, 1H), 8.35 (d, J = 6.0 Hz, 1H), 8.23 (s, 1H), 8.03- 7.90 (m, 2H), 7.50 (d, J = 8.0 Hz, 1H), 6.79 (s, 1H), 5.82-5.58 (m, 1H), 5.26-5.03 (m, 1H), 4.96-4.81 (m, 1H), 4.43-4.35 (m, 1H), 4.32-4.30 (m, 1H), 4.19-4.12 (m, 1H), 3.96-3.92 (m, 2H), 3.84-3.75 (m, 2H), 3.51 (s, 3H), 2.41-2.31 (m, 2H), 2.10 (s, 5H), 2.02- 1.84 (m, 4H), 1.71- 1.59 (m, 1H), 1.23 (s, 2H), 0.81-0.69 (m, |
| | phosphonic | | | 2H), 0.02 (d, J = 4.0 |
| | acid | | | Hz, 1H) |
| 375 | ((2- (((3S,6S,7aS, 8aR,9aR)-3- (3-(5- (difluoro- methyl) isoxazol-3- yl)azetidine- 1-carbonyl)- 5-oxodeca- hydro-1H- cyclopropa[d] pyrrolo[1,2- a]azocin-6- | 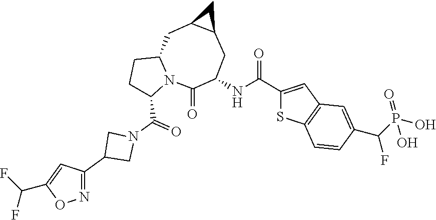 [SMILES: O=C(N[C@H]1C[C@@H]2C[C@@H]2C[C@H]2CC[C@@H](C(=O)N3CC(c4cc(C(F)F)on4)C3)N2C1=O)c1cc2cc(C(F)P(=O)(O)O)ccc2s1] | 667.1 | ¹H NMR (400 MHz, CD₃OD) δ 9.02-8.92 (m, 1H), 8.14-7.90 (m, 3H), 7.62-7.54 (m, 1H), 7.08-6.75 (m, 1H), 5.89-5.76 (m, 1H), 5.07-4.93 (m, 2H), 4.66 (t, J = 8.8 Hz, 1H), 4.52 (d, J = 8.0 Hz, 1H), 4.43- 4.28 (m, 2H), 4.22- 4.10 (m, 2H), 2.48- 2.42 (m, 1H), 2.34- 2.14 (m, 4H), 2.03- |
| | yl)carbamoyl) | | | 1.91 (m, 2H), 1.79- |
| | benzo[b] | | | 1.66 (m, 1H), 1.47- |
| | thiophen-5- | | | 1.30 (m, 1H), 1.01- |
| | yl)fluoro- | | | 0.89 (m, 1H), 0.86- |
| | methyl) | | | 0.83 (m, 1H), 0.10- |
| | phosphonic | | | 0.02 (m, 1H) |
| | acid | | | |
| 373 | (fluoro(2- (((3S,6S,7aS, 8aR,9aR)-5- oxo-3- (2,3,4,5- tetrahydro- benzo [b][1,4] oxazepine-5- carbonyl) decahydro-1H- cyclopropa[d] pyrrolo[1,2- a]azocin-6- yl)carbamoyl) benzo[b] thiophen-5- | 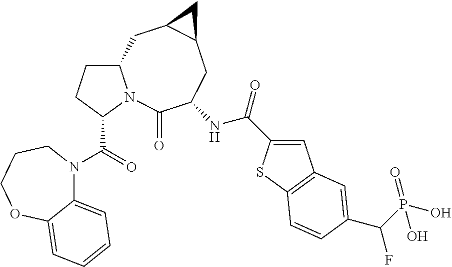 [SMILES: O=C(N[C@H]1C[C@@H]2C[C@@H]2C[C@H]2CC[C@@H](C(=O)N3CCCOc4ccccc43)N2C1=O)c1cc2cc(C(F)P(=O)(O)O)ccc2s1] | 642 | ¹H NMR (400 MHz, CD₃OD) δ 8.10 (s, 1H), 8.05-7.94 (m, 1H), 7.90-7.80 (m, 1H), 7.68-7.57 (m, 2H), 7.33-7.27 (m, 1H), 7.21-7.10 (m, 2H), 5.77-5.61 (m, 1H), 5.01 (t, J = 9.2 Hz, 1H), 4.77-4.57 (m, 3H), 4.46 (d, J = 12.0 Hz, 1H), 4.22- 4.13 (m, 1H), 3.67 (t, J = 11.6 Hz, 1H), 2.84- 2.74 (m, 1H), 2.54- 2.46 (m, 1H), 2.34- 2.28 (m, 1H), 2.20- |
| | yl)methyl) | | | 2.06 (m, 2H),2.06- |
| | phosphonic | | | 1.93 (m, 2H), 1.80- |
| | acid | | | 1.73 (m, 2H), 1.65- |
| | | | | 1.51 (m, 2H), 1.12- |
| | | | | 1.03 (m, 1H), 0.93- |
| | | | | 0.88 (m, 1H), 0.10- |
| | | | | 0.06 (m, 1H) |
| 372 | ((2- (((3S,6S,7aS, 8aR,9aR)-3- (3,4-dihydro- 2H- benzo[b][1,4] oxazine-4- carbonyl)-5- oxodecahydro- 1H- cyclopropa[d] pyrrolo[1,2- a]azocin-6- yl)carbamoyl) benzo[b] thiophen-5- yl)fluoro- methyl) | 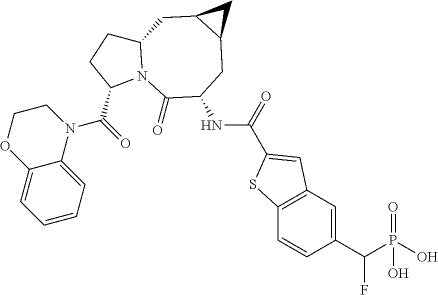 [SMILES: O=C(N[C@H]1C[C@@H]2C[C@@H]2C[C@H]2CC[C@@H](C(=O)N3CCOc4ccccc43)N2C1=O)c1cc2cc(C(F)P(=O)(O)O)ccc2s1] | 628 | ¹H NMR (400 MHz, CD₃OD) δ 7.98 (s, 2H), 7.83 (d, J = 8.4 Hz, 1H), 7.61 (dd, J = 4.0, 8.4 Hz, 1H), 7.46- 7.18 (m, 1H), 7.05 (t, J = 7.2 Hz, 1H), 6.91-6.84 (m, 2H), 5.73-5.57 (m, 1H), 5.19 (d, J = 2.0 Hz, 1H), 5.06-4.95 (m, 2H), 4.40-4.21 (m, 4H), 2.48-2.40 (m, 1H), 2.39-2.28 (m, 2H), 2.26-2.17 (m, 2H), 2.09 (d, J = 8.8 Hz, 1H), 2.00-1.92 (m, 1H), 1.82-1.71 |
| | phosphonic | | | (m, 1H), 1.52 (s, |
| | acid | | | 1H), 1.01-0.93 (m, |
| | | | | 1H), 0.90-0.84 (m, |
| | | | | 1H), 0.09-0.05 (m, |
| | | | | 1H) |
| 371 | ((S)- fluoro(2- (((3S,6S,7aR, 8aS,9aR)-3- (3-(6- methyl- pyridin-2- yl)azetidine- 1-carbonyl)- 5-oxodeca- hydro-1H- cyclopropa[d] pyrrolo[1,2- a]azocin-6- | 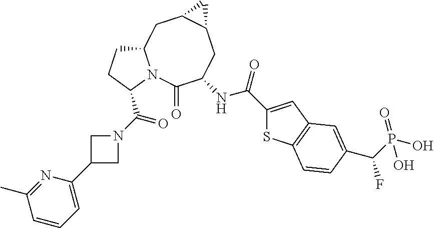 [SMILES: Cc1cccc(C2CN(C(=O)[C@@H]3CC[C@@H]4C[C@@H]5C[C@@H]5C[C@H](NC(=O)c5cc6cc([C@@H](F)P(=O)(O)O)ccc6s5)C(=O)N43)C2)n1] | 641.5 | ¹H NMR (400 MHz, CD₃OD) δ 8.31-8.16 (m, 1H), 8.10-8.00 (m, 2H), 8.00-7.78 (m, 2H), 7.65-7.53 (m, 2H), 5.87-5.68 (m, 1H), 4.93 (d, J = 5.2 Hz, 2H), 4.69- 4.58 (m, 1H), 4.42- 4.35 (m, 2H), 4.32 (t, J = 6.0 Hz, 1H), 4.22- 4.09 (m, 2H), 2.76- 2.57 (m, 3H), 2.43- 2.33 (m, 1H), 2.28- |
| | yl)carbamoyl) | | | 2.12 (m, 3H), 2.03- |
| | benzo[b] | | | 1.84 (m, 3H), 1.73- |
| | thiophen-5- | | | 1.56 (m, 1H), 1.54- |
| | yl)methyl) | | | 1.40 (m, 1H), 1.34- |
| | phosphonic | | | 1.21 (m, 1H), 0.93- |
| | acid | | | 0.79 (m, 1H), 0.18 (d, |
| | | | | J = 5.2 Hz, 1H) |
| 370 | ((R)- fluoro(2- (((3S,6S,7aR, 8aS,9aR)-3- (3-(6- methylpyridin- 2- yl)azetidine- 1-carbonyl)- 5-oxodeca- hydro-1H- cyclopropa[d] pyrrolo[1,2- a]azocin-6- | 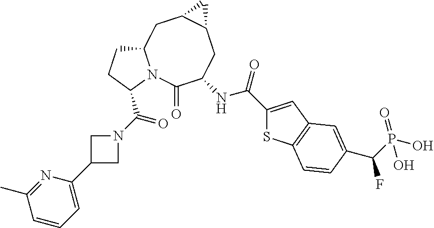 [SMILES: Cc1cccc(C2CN(C(=O)[C@@H]3CC[C@@H]4C[C@@H]5C[C@@H]5C[C@H](NC(=O)c5cc6cc([C@H](F)P(=O)(O)O)ccc6s5)C(=O)N43)C2)n1] | 641.5 | ¹H NMR (400 MHz, CD₃OD) δ 8.32-8.17 (m, 1H), 8.13-7.99 (m, 2H), 7.98-7.77 (m, 2H), 7.68-7.53 (m, 2H), 5.89-5.67 (m, 1H), 4.96 (d, J = 4.4 Hz, 2H), 4.66- 4.53 (m, 1H), 4.42- 4.30 (m, 3H), 4.20- 3.91 (m, 2H), 2.77- 2.57 (m, 3H), 2.46- 2.33 (m, 1H), 2.30- 2.11 (m, 3H), 2.05- |
| | yl)carbamoyl) | | | 1.82 (m, 3H), 1.75- |
| | benzo[b] | | | 1.59 (m, 1H), 1.45 |
| | thiophen-5- | | | (dd, J = 5.6, 11.6 Hz, |
| | yl)methyl) | | | 1H), 1.29 (d, J = 5.6 |
| | phosphonic | | | Hz, 1H), 0.86 (d, J = |
| | acid | | | 4.8 Hz, 1H), 0.22- |
| | | | | 0.14 (m, 1H) |
| 369 | ((2- (((3S,6S,7aS, 8aR,9aR)-3- (3-(4- fluorobenzyl) azetidine-1- carbonyl)-5- oxodecahydro- 1H- cyclopropa[d] pyrrolo[1,2- a]azocin-6- yl)carbamoyl) | 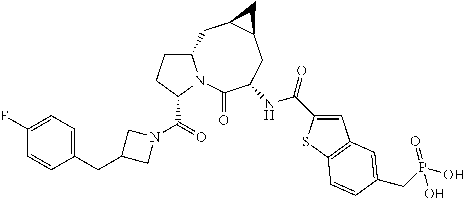 [SMILES: O=C(N[C@H]1C[C@@H]2C[C@@H]2C[C@H]2CC[C@@H](C(=O)N3CC(Cc4ccc(F)cc4)C3)N2C1=O)c1cc2cc(CP(=O)(O)O)ccc2s1] | 640.1 | ¹H NMR (400 MHz, DMSO-d₆) δ 8.82- 8.74 (m, 1H), 8.22 (d, J = 10.0 Hz, 1H), 7.90 (d, J = 3.6 Hz, 1H), 7.78 (s, 1H), 7.36 (d, J = 6.8 Hz, 1H), 7.25- 7.22 (m, 2H), 7.11- 7.06 (m, 2H), 4.96- 4.92 (m, 1H), 4.48- 4.30 (m, 2H), 4.15- 4.13 (m, 2H), 3.88- 3.82 (m, 2H), 3.12- |
| | benzo[b] | | | 3.07 (m, 2H), 2.89- |
| | thiophen-5- | | | 2.82 (m, 3H), 2.32- |
| | yl)methyl) | | | 2.30 (m, 1H), 2.10- |
| | phosphonic | | | 1.98 (m, 4H), 1.82- |
| | acid | | | 1.80 (m, 2H), 1.67- |
| | | | | 1.57 (m, 1H), 1.39- |
| | | | | 1.30 (m, 1H), 0.72- |
| | | | | 0.69 (m, 2H), 0.02- |
| | | | | 0.04 (m, 1H) |
| 368 | ((2- (((3S,6S,7aS, 8aR,9aR)-3- (3-(2- fluorobenzyl) azetidine-1- carbonyl)-5- oxodecahydro- 1H- cyclopropa[d] pyrrolo[1,2- a]azocin-6- yl)carbamoyl) | 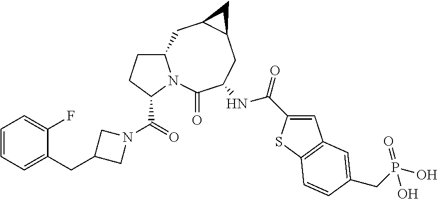 [SMILES: O=C(N[C@H]1C[C@@H]2C[C@@H]2C[C@H]2CC[C@@H](C(=O)N3CC(Cc4ccccc4F)C3)N2C1=O)c1cc2cc(CP(=O)(O)O)ccc2s1] | 640.3 | ¹H NMR (400 MHz, DMSO-d₆) δ 8.84- 8.70 (m, 1H), 8.17 (d, J = 8.6 Hz, 1H), 7.84- 7.78 (m, 1H), 7.71 (s, 1H), 7.34-7.20 (m, 3H), 7.17-7.05 (m, 2H), 4.91 (t, J = 8.4 Hz, 1H), 4.55- 4.22 (m, 2H), 4.16 (d, J = 8.8 Hz, 2H), 3.91 (t, J = 9.2 Hz, 1H), 3.86-3.81 (m, 1H), |
| | benzo[b] | | | 2.95-2.91 (m, 1H), |
| | thiophen-5- | | | 2.89-2.82 (m, 4H), |
| | yl)methyl) | | | 2.40-2.27 (m, 2H), |
| | phosphonic | | | 2.15-2.00 (m, 3H), |
| | acid | | | 1.87-1.75 (m, 2H), |
| | | | | 1.68-1.54 (m, 1H), |
| | | | | 1.41-1.29 (m, 1H), |
| | | | | 0.86-0.65 (m, 2H), |
| | | | | 0.03-0.09 (m, 1H) |
| 367 | (fluoro(2- (((3S,6S,7aS, 8aR,9aR)-3- (((3S,4S)-4- fluoro-1-(1- methyl-1H- imidazol-2- yl)pyrrolidin- 3-yl) carbamoyl)- 5- oxodecahydro- 1H- cyclopropa[d] pyrrolo[1,2- a]azocin-6- yl)carbamoyl) benzo[b] thiophen-5- yl)methyl) | 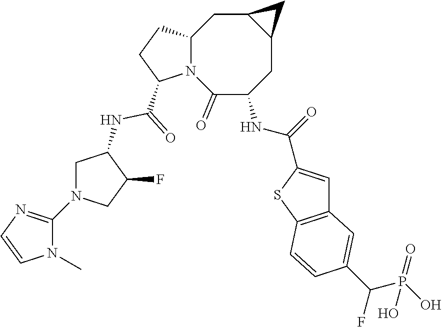 [SMILES: Cn1ccnc1N1C[C@H](NC(=O)[C@@H]2CC[C@@H]3C[C@H]4C[C@H]4C[C@H](NC(=O)c4cc5cc(C(F)P(=O)(O)O)ccc5s4)C(=O)N32)[C@@H](F)C1] | 677.1 | ¹H NMR (400 MHz, CD₃OD) δ 8.03 (s, 1H), 7.98 (s, 1H), 7.91-7.82 (m, 1H), 7.57 (d, J = 8.4 Hz, 1H), 6.89 (d, J = 8.4 Hz, 2H), 5.84-5.66 (m, 1H), 5.30-5.10 (m, 1H), 4.99 (t, J = 8.8 Hz, 1H), 4.53 (dd, J = 4.6, 11.4 Hz, 1H), 4.49-4.42 (m, 1H), 4.29 (d, J = 6.6 Hz, 1H), 4.18-4.05 (m, 1H), 4.05-3.99 (m, 1H), 3.98-3.84 (m, 1H), 3.72 (s, 3H), 3.69 (d, J = 10.6 Hz, 1H), 2.50-2.39 (m, 1H), 2.29 (d, J = 13.8 |
| | phosphonic | | | Hz, 1H), 2.23-2.15 |
| | acid | | | (m, 2H), 2.14-2.02 |
| | | | | (m, 2H), 2.02-1.91 |
| | | | | (m, 1H), 1.78-1.67 |
| | | | | (m, 1H), 1.29 (d, J = |
| | | | | 2.0 Hz, 1H), 0.96- |
| | | | | 0.81 (m, 2H), 0.13- |
| | | | | 0.05 (m, 1H) |
| 366 | (fluoro(2- (((3S,6S,7aS, 8aR,9aR)-3- (((3S,4S)-4- fluoro-1-(5- methyl-1H- 1,2,4-triazol- 3- yl)pyrrolidin- 3- yl)carbamoyl)- 5- oxodecahydro- 1H- cyclopropa[d] pyrrolo[1,2- a]azocin-6- yl)carbamoyl) benzo[b] thiophen-5- | 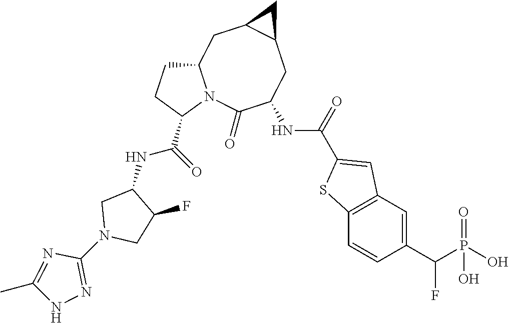 [SMILES: Cc1nc(N2C[C@H](NC(=O)[C@@H]3CC[C@@H]4C[C@H]5C[C@H]5C[C@H](NC(=O)c5cc6cc(C(F)P(=O)(O)O)ccc6s5)C(=O)N43)[C@@H](F)C2)n[nH]1] | 678.3 | ¹H NMR (400 MHz, DMSO-d₆) δ 8.82 (d, J = 7.2 Hz, 1H), 8.27- 8.21 (m, 2H), 8.02 (d, J = 8.4 Hz, 1H), 7.97 (s, 1H), 7.53 (d, J = 8.8 Hz, 1H), 5.90- 5.76 (m, 1H), 5.18- 5.04 (m, 1H), 4.89 (q, J = 8.4 Hz, 1H), 4.39- 4.34 (m, 2H), 4.17- 4.11 (m, 2H), 3.70- 3.67 (m, 2H), 3.30 (d, J = 10.4 Hz, 1H), 2.42- 2.29 (m, 2H), 2.22 (s, 3H), 2.17-2.09 (m, 2H), 1.97-1.88 (m, 3H), 1.69-1.59 (m, 1H), 1.22-1.13 (m, 1H), 0.80-0.69 |
| | yl)methyl) | | | (m, 2H), 0.02 (q, J = |
| | phosphonic | | | 4.8 Hz, 1H) |
| | acid | | | |
| 365 | ((2- (((3S,6S,7aS, 8aR,9aR)-3- (3-(hydroxy (phenyl) methyl) azetidine-1- carbonyl)-5- oxodecahydro- 1H- cyclopropa[d] pyrrolo[1,2- a]azocin-6- yl)carbamoyl) benzo[b] | 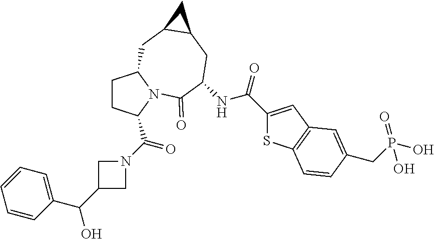 [SMILES: O=C(N[C@H]1C[C@@H]2C[C@@H]2C[C@H]2CC[C@@H](C(=O)N3CC(C(O)c4ccccc4)C3)N2C1=O)c1cc2cc(CP(=O)(O)O)ccc2s1] | 638.1 | ¹H NMR (400 MHz, CD₃OD) δ 8.01 (dd, J = 2.4, 13.6 Hz, 1H), 7.84 (d, J = 4.8 Hz, 1H), 7.80-7.74 (m, 1H), 7.46-7.43 (m, 1H), 7.38-7.26 (m, 4H), 7.26-7.18 (m, 1H), 5.05-4.98 (m, 1H), 4.80-4.77 (m, 1H), 4.57-4.41 (m, 2H), 4.34-4.20 (m, 1H), 4.08-3.89 (m, 1H), 3.75-3.67 (m, 1H), 3.15 (s, 1H), |
| | thiophen-5- | | | 3.10 (s, 1H), 3.02- |
| | yl)methyl) | | | 2.91 (m, 1H), 2.51- |
| | phosphonic | | | 2.39 (m, 1H), 2.33- |
| | acid | | | 2.00 (m, 4H), 1.98- |
| | | | | 1.88 (m, 1H), 1.77- |
| | | | | 1.67 (m, 1H), 1.43- |
| | | | | 1.39 (m, 1H), 1.37- |
| | | | | 1.33 (m, 2H), 0.98- |
| | | | | 0.92 (m, 1H), 0.87- |
| | | | | 0.79 (m, 1H), 0.10- |
| | | | | 0.01 (m, 1H) |
| 364 | ((2- (((3S,6S,7aS, 8aR,9aR)-5- oxo-3-(3- (pyrimidin-5- ylmethyl) azetidine-1- carbonyl) decahydro-1H- cyclopropa[d] pyrrolo[1,2- a]azocin-6- yl)carbamoyl) benzo[b] thiophen-5- yl)methyl) phosphonic acid | 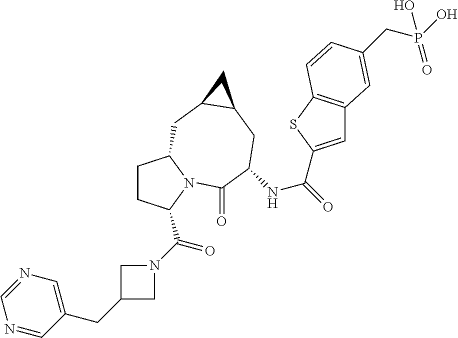 [SMILES: O=C(N[C@H]1C[C@@H]2C[C@@H]2C[C@H]2CC[C@@H](C(=O)N3CC(Cc4cncnc4)C3)N2C1=O)c1cc2cc(CP(=O)(O)O)ccc2s1] | 624.3 | ¹H NMR (400 MHz, CD₃OD) δ 9.10-8.94 (m, 1H), 8.92-8.54 (m, 2H), 8.02 (d, J = 12.8 Hz, 1H), 7.92- 7.68 (m, 2H), 7.45 (d, J = 8.0 Hz, 1H), 5.08- 4.98 (m, 2H), 4.52- 4.31 (m, 2H), 4.30- 4.06 (m, 2H), 4.05- 3.95 (m, 1H), 3.80- 3.62 (m, 1H), 3.20- 3.07 (m, 2H), 3.02 (s, 2H), 2.52-2.37 (m, 1H), 2.29 (d, J = 17.2 Hz, 1H), 2.24-2.06 (m, 3H), 1.91 (s, 2H), 1.81-1.66 (m, 1H), 1.49-1.32 (m, 1H), 1.00-0.89 (m, 1H), 0.88-0.77 (m, 1H), |
| | | | | 0.08-0.03 (m, J = 5.6 |
| | | | | Hz, 1H) |
| 358 | ((2- (((3S,6S,7aS, 8aR,9aR)-3- ((1-acetyl- 4,4-difluoro- pyrrolidin-3- yl)carbamoyl)- 5-oxodeca- hydro-1H- cyclopropa[d] pyrrolo[1,2- a]azocin-6- yl)carbamoyl) benzo[b] thiophen-5- yl)fluoro- methyl) phosphonic acid | 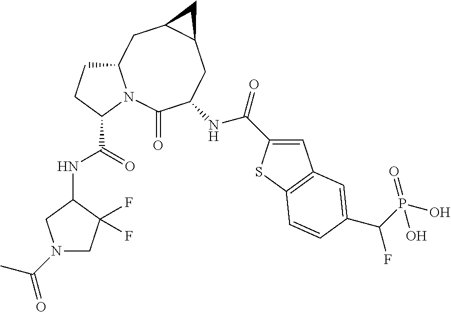 [SMILES: CC(=O)N1CC(NC(=O)[C@@H]2CC[C@@H]3C[C@H]4C[C@H]4C[C@H](NC(=O)c4cc5cc(C(F)P(=O)(O)O)ccc5s4)C(=O)N32)C(F)(F)C1] | 657.2 | ¹H NMR (400 MHz, DMSO-d₆) δ 8.91- 8.87 (m, 1H), 8.43- 8.34 (m, 1H), 8.26- 8.25 (m, 1H), 8.01 (d, J = 8.0 Hz, 1H), 7.96 (s, 1H), 7.52 (d, J = 8.4 Hz, 1H), 5.83- 5.77 (m, 1H), 4.93- 4.90 (m, 1H), 4.82- 4.65 (m, 1H), 4.53- 4.46 (m, 1H), 4.16 (d, J = 6.4 Hz, 1H), 4.00- 3.93 (m, 2H), 3.10- 3.08 (m, 1H), 2.97- 2.87 (m, 1H), 2.39- 2.38 (m, 1H), 2.15- 2.11 (m, 2H), 1.98- 1.97 (m, 1H), 1.94- |
| | | | | 1.92 (m, 3H), 1.86- |
| | | | | 1.84 (m, 1H), 1.69- |
| | | | | 1.60 (m, 1H), 1.23- |
| | | | | 1.22 (m, 1H), 1.16- |
| | | | | 1.13 (m, 1H), 0.85- |
| | | | | 0.83 (m, 1H), 0.77- |
| | | | | 0.67 (m, 2H), 0.04- |
| | | | | 0.02 (m, 1H) |
| 357 | ((S)-(2- (((3S,6S,7aR, 8aS,9aR)-3- (3-(4- ethylpyridin- 3- yl)azetidine- 1-carbonyl)- 5-oxodeca- hydro-1H- cyclopropa[d] pyrrolo[1,2- a]azocin-6- yl)carbamoyl) | 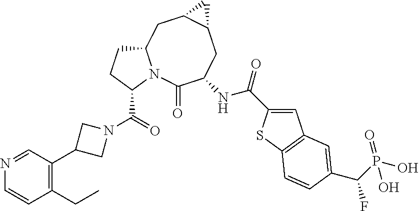 [SMILES: CCc1ccncc1C1CN(C(=O)[C@@H]2CC[C@@H]3C[C@@H]4C[C@@H]4C[C@H](NC(=O)c4cc5cc([C@@H](F)P(=O)(O)O)ccc5s4)C(=O)N32)C1] | 655.2 | ¹H NMR (400 MHz, CD₃OD) 8.69-8.52 (m, 1H), 8.28 (d, J = 5.6 Hz, 1H), 8.07- 7.90 (m, 2H), 7.89- 7.72 (m, 1H), 7.70- 7.53 (m, 1H), 7.49- 7.35 (m, 1H), 5.83- 5.56 (m, 1H), 4.94 (s, 2H), 4.74-4.61 (m, 1H), 4.56-4.36 (m, 2H), 4.32 (s, 1H), 4.26-4.16 (m, 1H), 4.14-3.98 (m, 1H), |
| | benzo[b] | | | 2.65 (q, J = 7.6 Hz, |
| | thiophen-5- | | | 2H), 2.37-2.42 (m, |
| | yl)fluoro- | | | 1H), 2.32-2.22 (m, |
| | methyl) | | | 1H), 2.22-2.11 (m, |
| | phosphonic | | | 2H), 2.07-1.81 (m, |
| | acid | | | 3H), 1.77-1.60 (m, |
| | | | | 1H), 1.54-1.38 (m, |
| | | | | 1H), 1.37-1.23 (m, |
| | | | | 2H), 1.23-1.11 (m, |
| | | | | 2H), 0.89-0.83 (m, |
| | | | | 1H), 0.19 (d, J = 2.8 |
| | | | | Hz, 1H) |
| 356 | ((S)-(2- (((3S,6S,7aR, 8aS,9aR)-3- (3-cyano-3- (pyridin-2- yl)azetidine- 1-carbonyl)- 5-oxodeca- hydro-1H- cyclopropa[d] pyrrolo[1,2- a]azocin-6- yl)carbamoyl) | 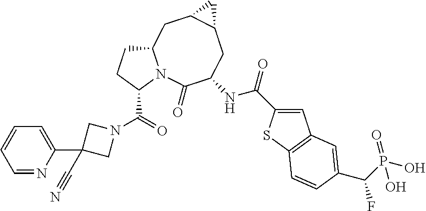 [SMILES: N#CC1(c2ccccn2)CN(C(=O)[C@@H]2CC[C@@H]3C[C@@H]4C[C@@H]4C[C@H](NC(=O)c4cc5cc([C@@H](F)P(=O)(O)O)ccc5s4)C(=O)N32)C1] | 652.2 | ¹H NMR (400 MHz, CD₃OD) δ 8.73-8.56 (m, 1H), 8.08-7.96 (m, 2H), 7.95-7.80 (m, 2H), 7.72 (d, J = 8.0 Hz, 1H), 7.66- 7.58 (m, 1H), 7.48- 7.33 (m, 1H), 5.77- 5.61 (m, 1H), 5.15- 5.11 (m, 1H), 4.98- 4.96 (m, 1H), 4.92 (s, 1H), 4.69-4.62 (m, 1H), 4.55-4.42 (m, |
| | benzo[b] | | | 2H), 4.37-4.29 (m, |
| | thiophen-5- | | | 1H), 2.41-2.29 (m, |
| | yl)fluoro- | | | 2H), 2.23-2.13 (m, |
| | methyl) | | | 2H), 2.04-1.88 (m, |
| | phosphonic | | | 3H), 1.74-1.59 (m, |
| | acid | | | 1H), 1.51-1.41 (m, |
| | | | | 1H), 1.32-1.25 (m, |
| | | | | 1H), 0.90-0.81 (m, |
| | | | | 1H), 0.24-0.17 (m, |
| | | | | 1H) |
| 355 | ((R)-(2- (((3S,6S,7aR, 8aS,9aR)-3- (3-cyano-3- (pyridin-2- yl)azetidine- 1-carbonyl)- 5-oxodeca- hydro-1H- cyclopropa[d] pyrrolo[1,2- a]azocin-6- yl)carbamoyl) | 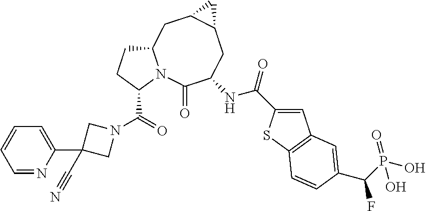 [SMILES: N#CC1(c2ccccn2)CN(C(=O)[C@@H]2CC[C@@H]3C[C@@H]4C[C@@H]4C[C@H](NC(=O)c4cc5cc([C@H](F)P(=O)(O)O)ccc5s4)C(=O)N32)C1] | 652.2 | ¹H NMR (400 MHz, CD₃OD) δ 8.71-8.55 (m, 1H), 8.11-7.96 (m, 2H), 7.94-7.81 (m, 2H), 7.73 (d, J = 8.0 Hz, 1H), 7.65- 7.58 (m, 1H), 7.47- 7.34 (m, 1H), 5.79- 5.63 (m, 1H), 5.13 (m, 1H), 4.97-4.94 (s, 1H), 4.92-4.90 (m, 1H), 4.68-4.64 (m, 1H), 4.54-4.42 |
| | benzo[b] | | | (m, 2H), 4.35-4.30 |
| | thiophen-5- | | | (m, 1H), 2.40-2.27 |
| | yl)fluoro- | | | (m, 2H), 2.21-2.13 |
| | methyl) | | | (m, 2H), 2.03-1.90 |
| | phosphonic | | | (m, 3H), 1.67 (m, |
| | acid | | | 1H), 1.50-1.40 (m, |
| | | | | 1H), 1.32-1.27 (m, |
| | | | | 1H), 0.90-0.82 (m, |
| | | | | 1H), 0.22-0.16 m, |
| | | | | 1H) |
| 354 | ((S)- fluoro(2- (((3S,6S,7aR, 8aS,9aR)-3- (3-(4- methoxy- pyridin-3- yl)azetidine- 1-carbonyl)- 5-oxodeca- hydro-1H- cyclopropa[d] pyrrolo[1,2- | 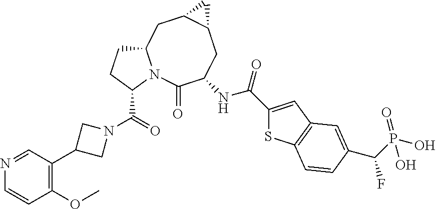 [SMILES: COc1ccncc1C1CN(C(=O)[C@@H]2CC[C@@H]3C[C@@H]4C[C@@H]4C[C@H](NC(=O)c4cc5cc([C@@H](F)P(=O)(O)O)ccc5s4)C(=O)N32)C1] | 657.2 | ¹H NMR (400 MHz, CD₃OD) δ 8.52-8.32 (m, 2H), 8.03-7.91 (m, 2H), 7.90-7.83 (m, 1H), 7.69-7.54 (m, 1H), 7.49-7.40 (m, 1H), 5.84-5.66 (m, 1H), 5.00-4.89 (m, 3H), 4.67-4.58 (m, 1H), 4.44-4.37 (m, 1H), 4.32 (d, J = 7.2 Hz, 2H), 4.14 (s, 1H), 4.12 (s, 3H), |
| | a]azocin-6- | | | 2.43-2.34 (m, 1H), |
| | yl)carbamoyl) | | | 2.31-2.22 (m, 1H), |
| | benzo[b] | | | 2.20-2.10 (m, 2H), |
| | thiophen-5- | | | 2.05-1.94 (m, 2H), |
| | yl)methyl) | | | 1.91-1.75 (m, 1H), |
| | phosphonic | | | 1.72-1.59 (m, 1H), |
| | acid | | | 1.48-1.38 (m, 1H), |
| | | | | 1.37-1.22 (m, 1H), |
| | | | | 0.90-0.83 (m, 1H), |
| | | | | 0.18 (q, J = 4.8 Hz, |
| | | | | 1H) |
| 353 | ((R)- fluoro(2- (((3S,6S,7aR, 8aS,9aR)-3- (3-(4- methoxy- pyridin-3- yl)azetidine- 1-carbonyl)- 5-oxodeca- hydro-1H- cyclopropa[d] pyrrolo[1,2- a]azocin-6- yl)carbamoyl) | 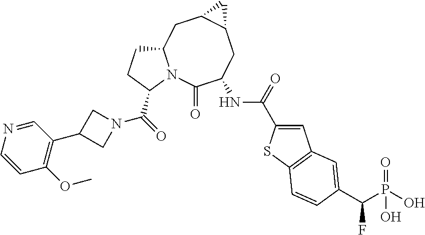 [SMILES: COc1ccncc1C1CN(C(=O)[C@@H]2CC[C@@H]3C[C@@H]4C[C@@H]4C[C@H](NC(=O)c4cc5cc([C@H](F)P(=O)(O)O)ccc5s4)C(=O)N32)C1] | 657.2 | ¹H NMR (400 MHz, CD₃OD) δ 8.57-8.34 (m, 2H), 8.08-7.92 (m, 2H), 7.88 (dd, J = 2.8, 8.4 Hz, 1H), 7.58 (d, J = 8.0 Hz, 1H), 7.52-7.42 (m, 1H), 5.87-5.64 (m, 1H), 5.01-4.92 (m, 3H), 4.64 (d, J = 7.0 Hz, 1H), 4.43-4.37 (m, 1H), 4.36-4.29 (m, 2H), 4.16 (s, 1H), 4.15-4.09 (m, 3H), 2.43-2.34 (m, 1H), |
| | benzo[b] | | | 2.32-2.23 (m, 1H), |
| | thiophen-5- | | | 2.21 (s, 2H), 2.06- |
| | yl)methyl) | | | 1.97 (m, 2H), 1.93- |
| | phosphonic | | | 1.79 (m, 1H), 1.75- |
| | acid | | | 1.61 (m, 1H), 1.49- |
| | | | | 1.41 (m, 1H), 1.37- |
| | | | | 1.27 (m, 1H), 0.91- |
| | | | | 0.84 (m, 1H), 0.21- |
| | | | | 0.16 (m, 1H) |
| 352 | ((2- (((3S,6S,7aS, 8aR,9aR)-5- oxo-3-(3- (pyridin-4- ylmethyl) azetidine-1- carbonyl) decahydro-1H- cyclopropa[d] pyrrolo[1,2- a]azocin-6- yl)carbamoyl) benzo[b] | 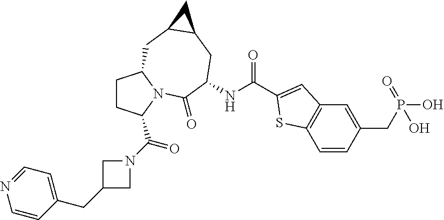 [SMILES: O=C(N[C@H]1C[C@@H]2C[C@@H]2C[C@H]2CC[C@@H](C(=O)N3CC(Cc4ccncc4)C3)N2C1=O)c1cc2cc(CP(=O)(O)O)ccc2s1] | 623.2 | ¹H NMR (400 MHz, CD₃OD) δ 8.68 (d, J = 6.4 Hz, 1H), 8.55 (d, J = 6.4 Hz, 1H), 8.02 (d, J = 4.4 Hz, 1H), 7.87 (J = 6.4, 17.6 Hz, 2H), 7.82- 7.77 (m, 2H), 7.41 (d, J = 8.4 Hz, 1H), 5.02 (J = 5.6, 8.4 Hz, 1H), 4.51-4.43 (m, 1H), 4.41-4.33 (m, 1H), 4.32-4.26 (m, 1H), 4.18-4.00 (m, 2H), |
| | thiophen-5- | | | 3.82-3.66 (m, 1H), |
| | yl)methyl) | | | 3.28 (d, J = 5.2 Hz, |
| | phosphonic | | | 2H), 3.27-3.20 (m, |
| | acid | | | 2H), 3.15-3.05 (m, |
| | | | | 1H), 2.55-2.41 (m, |
| | | | | 1H), 2.31 (J = 1.2, |
| | | | | 14.8 Hz, 1H), 2.27- |
| | | | | 2.15 (m, 2H), 2.15- |
| | | | | 2.06 (m, 1H), 2.03- |
| | | | | 1.91 (m, 2H), 1.81- |
| | | | | 1.69 (m, 1H), 1.47- |
| | | | | 1.35 (m, 1H), 1.02- |
| | | | | 0.90 (m, 1H), 0.88- |
| | | | | 0.79 (m, 1H), 0.11- |
| | | | | 0.02 (m, 1H) |
| 351 | ((2- (((3S,6S,7aS, 8aR,9aR)-5- oxo-3-(3- (pyridin-2- yloxy) azetidine-1- carbonyl) decahydro-1H- cyclopropa[d] pyrrolo[1,2- a]azocin-6- yl)carbamoyl) benzo[b] | 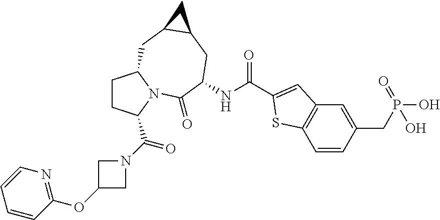 [SMILES: O=C(N[C@H]1C[C@@H]2C[C@@H]2C[C@H]2CC[C@@H](C(=O)N3CC(Oc4ccccn4)C3)N2C1=O)c1cc2cc(CP(=O)(O)O)ccc2s1] | 625.4 | 1H NMR (400 MHz, CD₃OD) δ 8.11-7.93 (m, 2H), 7.85-7.81 (m, 1H), 7.77 (d, J = 7.6 Hz, 1H), 7.74- 7.64 (m, 1H), 7.41 (t, J = 9.2 Hz, 1H), 7.00- 6.90 (m, 1H), 6.88- 6.79 (m, 1H), 5.42- 5.34 (m, 1H), 5.07- 5.00 (m, 1H), 4.72- 4.57 (m, 1H), 4.54- 4.51 (m, 1H), 4.47- 4.22 (m, 3H), 4.05- |
| | thiophen-5- | | | 3.90 (m, 1H), 3.27 (d, |
| | yl)methyl) | | | J = 5.6 Hz, 1H), 3.22 |
| | phosphonic | | | (d, J = 5.6 Hz, 1H), |
| | acid | | | 2.51-2.41 (m, 1H), |
| | | | | 2.34-2.24 (m, 2H), |
| | | | | 2.23-2.11 (m, 2H), |
| | | | | 2.01-1.90 (m, 2H), |
| | | | | 1.78-1.67 (m, 1H), |
| | | | | 1.46-1.35 (m, 1H), |
| | | | | 0.98-0.90 (m, 1H), |
| | | | | 0.87-0.81 (m, 1H), |
| | | | | 0.08-0.01 (m, 1H) |
| 350 | ((2- (((3S,6S,7aS, 8aR,9aR)-5- oxo-3-(3- (pyridin-3- yloxy) azetidine-1- carbonyl) decahydro-1H- cyclopropa[d] pyrrolo[1,2- a]azocin-6- yl)carbamoyl) benzo[b] | 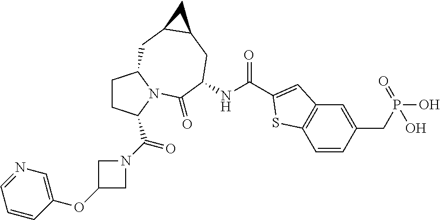 [SMILES: O=C(N[C@H]1C[C@@H]2C[C@@H]2C[C@H]2CC[C@@H](C(=O)N3CC(Oc4cccnc4)C3)N2C1=O)c1cc2cc(CP(=O)(O)O)ccc2s1] | 625.2 | 1H NMR (400 MHz, CD₃OD) δ = 8.35 (s, 1H), 8.15-7.99 (m, 1H), 7.86-7.79 (m, 2H), 7.76-7.73 (m, 1H), 7.65-7.46 (m, 2H), 7.43-7.31 (m, 1H), 5.23-5.10 (m, 1H), 5.05-5.01 (m, 1H), 4.84-4.79 (m, 1H), 4.64-4.55 (m, 1H), 4.53-4.47 (m, 1H), 4.40-4.30 (m, 2H), 4.13-3.98 (m, |
| | thiophen-5- | | | 1H), 3.28-3.18 (m, |
| | yl)methyl) | | | 2H), 2.50-2.43 (m, |
| | phosphonic | | | 1H), 2.36-2.84 (m, |
| | acid | | | 4H), 2.05-1.93 (m, |
| | | | | 2H), 1.82-1.70 (m, |
| | | | | 1H), 1.43 (s, 1H), |
| | | | | 1.39-1.35 (m, 2H), |
| | | | | 1.00-0.91 (m, 1H), |
| | | | | 0.88-0.79 (m, 1H), |
| | | | | 0.08 (d, J = 4.4 Hz, |
| | | | | 1H) |
| 349 | ((2- (((3S,6S,7aS, 8aR,9aR)-3- (3-(3- fluorobenzyl) azetidine-1- carbonyl)-5- oxodecahydro- 1H- cyclopropa[d] pyrrolo[1,2- a]azocin-6- yl)carbamoyl) benzo[b] thiophen-5- | 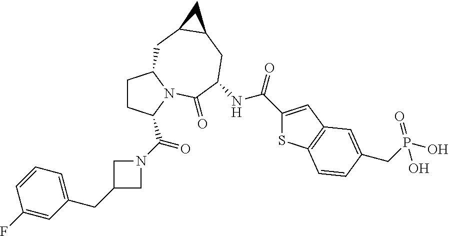 [SMILES: O=C(N[C@H]1C[C@@H]2C[C@@H]2C[C@H]2CC[C@@H](C(=O)N3CC(Cc4cccc(F)c4)C3)N2C1=O)c1cc2cc(CP(=O)(O)O)ccc2s1] | 640.3 | ¹H NMR (400 MHz, CD₃OD) 8.04 (d, J = 10.0 Hz, 1H), 7.88- 7.77 (m, 2H), 7.42 (d, J = 8.0 Hz, 1H), 7.33- 7.20 (m, 1H), 7.07- 6.86 (m, 3H), 5.03 (q, J = 8.8 Hz, 1H), 4.49- 4.45 (m, J = 7.6, 7.9 Hz, 1H), 4.31-4.23 (m, 2H), 4.13-3.90 (m, 2H), 3.75-3.61 (m, 1H), 3.28 (s, 1H), 3.22 (s, 1H), 2.96 (s, 3H), 2.51-2.37 (m, |
| | yl)methyl) | | | 1H), 2.33-2.26 (m, |
| | phosphonic | | | 1H), 2.25-2.05 (m, |
| | acid | | | 3H), 2.02-1.88 (m, |
| | | | | 2H), 1.78-1.67 (m, |
| | | | | 1H), 1.47-1.34 (m, |
| | | | | 1H), 1.01-0.89 (m, |
| | | | | 1H), 0.88-0.79 (m, |
| | | | | 1H), 0.05 (m, J = 10.4 |
| | | | | Hz, 1H) |
| 348 | ((2- (((3S,6S,7aS, 8aR,9aR)-3- (((3S,4S)-1- (1,4- dimethyl-1H- imidazol-2- yl)-4- fluoro- pyrrolidin-3- yl)carbamoyl)- 5-oxodeca- hydro-1H- cyclopropa[d] pyrrolo[1,2- a]azocin-6- | 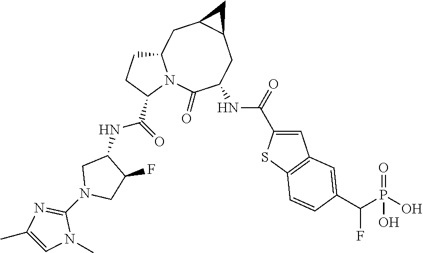 [SMILES: Cc1cn(C)c(N2C[C@H](NC(=O)[C@@H]3CC[C@@H]4C[C@H]5C[C@H]5C[C@H](NC(=O)c5cc6cc(C(F)P(=O)(O)O)ccc6s5)C(=O)N43)[C@@H](F)C2)n1] | 691.3 | ¹H NMR (400 MHz, DMSO-d₆) δ 8.83 (d, J = 1.6 Hz, 1H), 8.38- 8.29 (m, 1H), 8.22 (s, 1H), 7.90 (d, J = 7.2 Hz, 2H), 7.48 (d, J = 8.4 Hz, 1H), 6.55 (s, 1H), 5.65-5.45 (m, 1H), 5.14-4.98 (m, 1H), 4.90 (q, J = 8.4 Hz, 1H), 4.40-4.30 (m, 2H), 4.16-4.12 (m, 1H), 3.81-3.75 (m, 2H), 3.67-3.55 (m, 2H), 3.47 (s, 3H), 3.23 (d, J = 10.0 Hz, |
| | yl)carbamoyl) | | | 1H), 2.43-2.32 (m, |
| | benzo[b] | | | 2H), 2.19-2.09 (m, |
| | thiophen-5- | | | 2H), 1.99-1.93 (m, |
| | yl)fluoro- | | | 5H), 1.84 (dd, J = 8.8, |
| | methyl) | | | 15.2 Hz, 1H), 1.70- |
| | phosphonic | | | 1.59 (m, 1H), 1.22- |
| | acid | | | 1.11 (m, 1H), 0.80- |
| | | | | 0.66 (m, 2H), 0.02- |
| | | | | 0.08 (m, 1H) |
| 347 | (fluoro(2- (((3S,6S,7aS, 8aR,9aR)-3- (((3S,4S)-4- fluoro-1- (1H-1,2,4- triazol-3- yl)pyrrolidin- 3-yl) carbamoyl)- 5-oxodeca- hydro-1H- cyclopropa[d] pyrrolo[1,2- a]azocin-6- yl)carbamoyl) benzo[b] thiophen-5- | 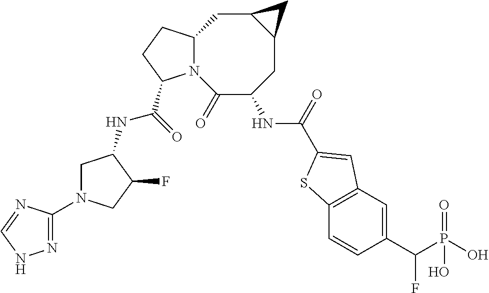 [SMILES: O=C(N[C@H]1C[C@@H]2C[C@@H]2C[C@H]2CC[C@@H](C(=O)N[C@H]3CN(c4nc[nH]n4)C[C@@H]3F)N2C1=O)c1cc2cc(C(F)P(=O)(O)O)ccc2s1] | 664.3 | ¹H NMR (400 MHz, DMSO-d₆) δ 8.83 (d, J = 7.2 Hz, 1H), 8.28- 8.20 (m, 2H), 8.02 (d, J = 8.4 Hz, 1H), 7.97 (s, 2H), 7.56- 7.48 (m, 1H), 5.90- 5.76 (m, 1H), 5.18- 5.04 (m, 1H), 4.93- 4.86 (m, 1H), 4.39- 4.33 (m, 2H), 4.17- 4.10 (m, 2H), 3.69- 3.65 (m, 2H), 3.32 (d, J = 10.4 Hz, 1H), 2.40- 2.30 (m, 2H), 2.17- 2.09 (m, 2H), 1.97- 1.79 (m, 3H), 1.69- 1.57 (m, 1H), 1.23- |
| | yl)methyl) | | | 1.12 (m, 1H), 0.80- |
| | phosphonic | | | 0.67 (m, 2H), 0.56- |
| | acid | | | 0.01 (m, 1H) |
| 346 | ((R)- fluoro(2- (((3S,6S,7aR, 8aS,9aR)-3- (3-(isoxazol- 3- yl)azetidine- 1-carbonyl)- 5-oxodeca- hydro-1H- cyclopropa[d] pyrrolo[1,2- a]azocin-6- yl)carbamoyl) benzo[b] thiophen-5- yl)methyl) phosphonic | 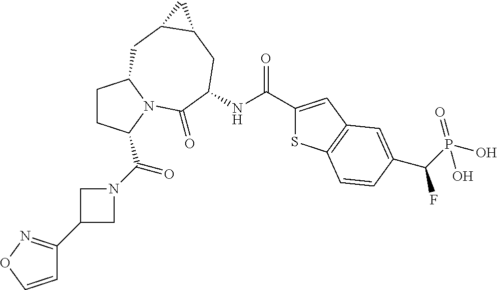 [SMILES: O=C(N[C@H]1C[C@H]2C[C@H]2C[C@H]2CC[C@@H](C(=O)N3CC(c4ccon4)C3)N2C1=O)c1cc2cc([C@H](F)P(=O)(O)O)ccc2s1] | 617.1 | ¹H NMR (400 MHz, CD₃OD) δ 8.63 (dd, J = 1.6, 17.6 Hz, 1H), 8.13-8.00 (m, 2H), 7.99-7.86 (m, 1H), 7.69-7.53 (m, 1H), 6.79-6.55 (m, 1H), 5.96-5.73 (m, 1H), 4.98-4.92 (m, 1H), 4.70-4.57 (m, 1H), 4.53-4.39 (m, 2H), 4.39-4.27 (m, 2H), 4.17-4.00 (m, 2H), 2.40-2.30 (m, 1H), 2.30-2.21 (m, 1H), 2.20-2.11 (m, 2H), 2.05-1.93 (m, 2H), 1.91-1.80 (m, 1H), |
| | acid | | | 1.76-1.65 (m, 1H), |
| | | | | 1.53-1.39 (m, 1H), |
| | | | | 1.35-1.21 (m, 1H), |
| | | | | 0.93-0.79 (m, 1H), |
| | | | | 0.23-0.13 (m, 1H) |
| 345 | ((R)-(2- (((3S,6S,7aR, 8aS,9aR)-3- (3-(4- ethylpyridin- 3- yl)azetidine- 1-carbonyl)- 5-oxodeca- hydro-1H- cyclopropa[d] pyrrolo[1,2- a]azocin-6- yl)carbamoyl) benzo[b] thiophen-5- yl)fluoro- methyl) phosphonic | 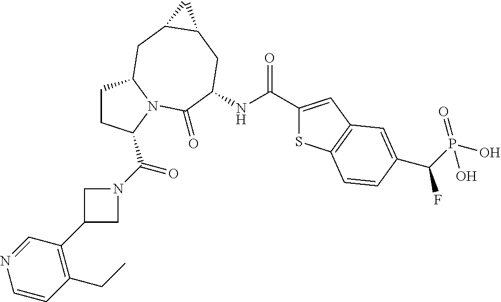 [SMILES: CCc1ccncc1C1CN(C(=O)[C@@H]2CC[C@@H]3C[C@@H]4C[C@@H]4C[C@H](NC(=O)c4cc5cc([C@H](F)P(=O)(O)O)ccc5s4)C(=O)N32)C1] | 655.3 | ¹H NMR (400 MHz, CD₃OD) δ 8.49 (d, J = 8.4 Hz, 1H), 8.15- 8.21(m, 1H), 8.06- 7.92 (m, 1H), 7.92- 7.71 (m, 2H), 7.64- 7.52 (m, 1H), 7.37- 7.89 (m, 1H), 5.76- 5.58 (m, 1H), 4.97- 4.90 (m, 2H), 4.71- 4.57 (m, 1H), 4.54- 4.34 (m, 2H), 4.33- 4.23 (m, 1H), 4.20- 3.96 (m, 2H), 2.69- 2.55 (m, 2H), 2.47- 2.34 (m, 1H), 2.33- 2.24 (m, 1H), 2.22- 2.08 (m, 2H), 2.06- 1.94 (m, 2H), 1.93- |
| | acid | | | 1.81 (m, 1H), 1.77- |
| | | | | 1.58 (m, 1H), 1.50- |
| | | | | 1.39 (m, 1H), 1.33- |
| | | | | 1.24 (m, 1H), 1.23- |
| | | | | 1.11 (m, 3H), 0.93- |
| | | | | 0.79 (m, 1H), 0.24- |
| | | | | 0.13 (m, 1H) |
| 344 | ((S)- fluoro(2- (((3S,6S,7aR, 8aS,9aR)-3- (3-(isoxazol- 3- yl)azetidine- 1-carbonyl)- 5-oxodeca- hydro-1H- cyclopropa[d] pyrrolo[1,2- a]azocin-6- yl)carbamoyl) benzo[b] thiophen-5- yl)methyl) phosphonic | 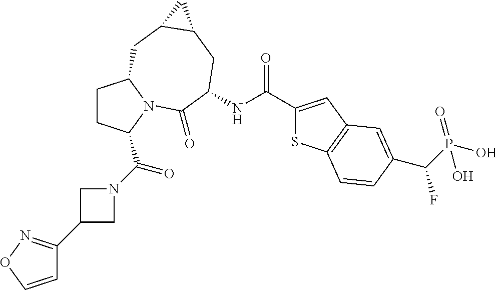 [SMILES: O=C(N[C@H]1C[C@H]2C[C@H]2C[C@H]2CC[C@@H](C(=O)N3CC(c4ccon4)C3)N2C1=O)c1cc2cc([C@@H](F)P(=O)(O)O)ccc2s1] | 617.2 | ¹H NMR (400 MHz, CD₃OD) δ 8.67-8.58 (m, 1H), 8.11-8.06 (m, 1H), 8.06-8.02 (m, 1H), 7.95 (dd, J = 4.4, 8.4 Hz, 1H), 7.65- 7.57 (m, 1H), 6.78- 6.54 (m, 1H), 5.94- 5.74 (m, 1H), 4.97- 4.93 (m, 1H), 4.70- 4.59 (m, 1H), 4.53- 4.40 (m, 2H), 4.40- 4.35 (m, 1H), 4.35- 4.29 (m, 1H), 4.16- 4.02 (m, 2H), 2.40- 2.31 (m, 1H), 2.30- 2.21 (m, 1H), 2.20- 2.11 (m, 2H), 2.05- |
| | acid | | | 1.92 (m, 2H), 1.86 |
| | | | | (m, 1H), 1.77-1.65 |
| | | | | (m, 1H), 1.53-1.40 |
| | | | | (m, 1H), 1.36-1.22 |
| | | | | (m, 1H), 0.85 (m, |
| | | | | 1H), 0.21-0.15 (m, |
| | | | | 1H) |
| 343 | ((2- (((3S,6S,7aR, 8aS,9aR)-3- (3-(4- ethylpyridin- 3- yl)azetidine- 1-carbonyl)- 5-oxodeca- hydro-1H- cyclopropa[d] pyrrolo[1,2- a]azocin-6- yl)carbamoyl) benzo[b] thiophen-5- yl)methyl) phosphonic acid | 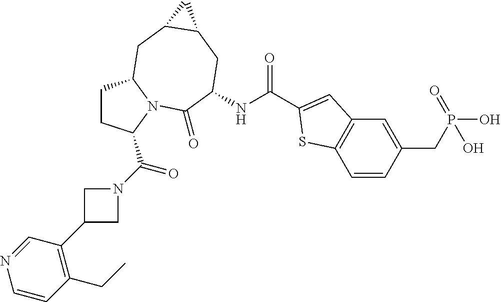 [SMILES: CCc1ccncc1C1CN(C(=O)[C@@H]2CC[C@@H]3C[C@@H]4C[C@@H]4C[C@H](NC(=O)c4cc5cc(CP(=O)(O)O)ccc5s4)C(=O)N32)C1] | 637.3 | ¹H NMR (400 MHz, CD₃OD) 8.48 (s, 1H), 8.28-8.06 (m, 1H), 7.98-7.87 (m, 1H), 7.81-7.54 (m, 2H), 7.47-7.22 (m, 2H), 4.96-4.90 (m, 2H), 4.71-4.55 (m, 1H), 4.52-4.41 (m, 1H), 4.40-4.23 (m, 2H), 4.20-4.10 (m, 1H), 4.09-3.97 (m, 1H), 3.19-3.03 (m, 2H), 2.59 (s, 2H), 2.36- 2.40 (m, 1H), 2.33- 2.24 (m, 1H), 2.24- 2.08 (m, 2H), 2.07- 1.94 (m, 2H), 1.93- 1.80 (m, 1H), 1.76- |
| | | | | 1.60 (m, 1H), 1.52- |
| | | | | 1.37 (m, 1H), 1.34- |
| | | | | 1.11 (m, 4H), 0.83- |
| | | | | 0.89 (m, 1H), 0.27- |
| | | | | 0.09 (m, 1H) |
| 342 | ((2- (((3S,6S,7aS, 8aR,9aR)-5- oxo-3-(3- (pyrimidin-2- yloxy) azetidine-1- carbonyl) decahydro-1H- cyclopropa[d] pyrrolo[1,2- a]azocin-6- yl)carbamoyl) benzo[b] | 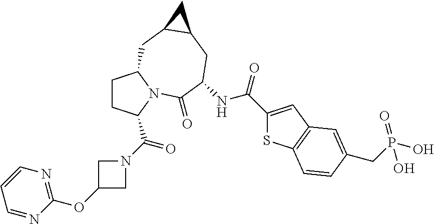 [SMILES: O=C(N[C@H]1C[C@@H]2C[C@@H]2C[C@H]2CC[C@@H](C(=O)N3CC(Oc4ncccn4)C3)N2C1=O)c1cc2cc(CP(=O)(O)O)ccc2s1] | 626.1 | ¹H NMR (400 MHz, CD₃OD) δ 8.57 (d, J = 4.8 Hz, 1H), 8.46 (d, J = 4.0 Hz, 1H), 8.04- 7.80 (m, 2H), 7.75 (s, 1H), 7.41 (dd, J = 8.4, 13.2 Hz, 1H), 7.17- 6.97 (m, 1H), 5.50- 5.35 (m, 1H), 5.08- 4.97 (m, 2H), 4.76- 4.63 (m, 1H), 4.57- 4.49 (m, 1H), 4.39- 4.25 (m, 2H), 4.13- 3.94 (m, 1H), 3.27 (d, |
| | thiophen-5- | | | J = 5.4 Hz, 1H), 3.22 |
| | yl)methyl) | | | (d, J = 5.6 Hz, 1H), |
| | phosphonic | | | 2.51-2.40 (m, 1H), |
| | acid | | | 2.31 (d, J = 15.0 Hz, |
| | | | | 2H), 2.24-2.11 (m, |
| | | | | 2H), 2.05-1.92 (m, |
| | | | | 2H), 1.80-1.68 (m, |
| | | | | 1H), 1.48-1.36 (m, |
| | | | | 1H), 1.00-0.89 (m, |
| | | | | 1H), 0.88-0.78 (m, |
| | | | | 1H), 0.11-0.03 (m, |
| | | | | 1H) |
| 341 | (fluoro(2- (((3S,6S,7aS, 8aR,9aR)-3- (3-((S- methyl- sulfonimidoyl) methyl) azetidine-1- carbonyl)-5- oxodecahydro- 1H- cyclopropa[d] pyrrolo[1,2- a]azocin-6- yl)carbamoyl) benzo[b] | 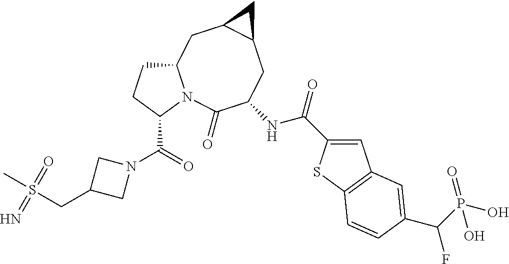 [SMILES: CS(=N)(=O)CC1CN(C(=O)[C@@H]2CC[C@@H]3C[C@H]4C[C@H]4C[C@H](NC(=O)c4cc5cc(C(F)P(=O)(O)O)ccc5s4)C(=O)N32)C1] | 641.2 | ¹H NMR (400 MHz, CD₃OD) δ 8.11 (s, 1H), 8.03 (s, 1H), 7.93 (d, J = 8.4 Hz, 1H), 7.59 (d, J = 8.8 Hz, 1H), 5.89-5.70 (m, 1H), 5.01 (J = 3.6, 8.8 Hz, 1H), 4.83 (d, J = 8.4 Hz, 1H), 4.53-4.41 (m, 2H), 4.32-4.25 (m, 1H), 4.23-4.06 (m, 3H), 3.99-3.81 (m, 1H), 3.59-3.50 (m, 3H), 3.37 (d, J = 5.6 Hz, 1H), 2.53-2.39 (m, |
| | thiophen-5- | | | 1H), 2.35-2.07 (m, |
| | yl)methyl) | | | 4H), 2.04-1.90 (m, |
| | phosphonic | | | 2H), 1.83-1.66 (m, |
| | acid | | | 1H), 1.44-1.35 (m, |
| | | | | 1H), 0.94 (J = 8.0, |
| | | | | 13.2 Hz, 1H), 0.88- |
| | | | | 0.81 (m, 1H), 0.14- |
| | | | | 0.02 (m, 1H) |
| 340 | ((2- (((3S,6S,7aS, 8aR,9aR)-3- (((S)-1- carbamoyl- 4,4- difluoro- pyrrolidin-3- yl) carbamoyl)-5- oxodecahydro- 1H- cyclopropa[d] pyrrolo[1,2- a]azocin-6- yl)carbamoyl) benzo[b] thiophen-5- yl)fluoro- | 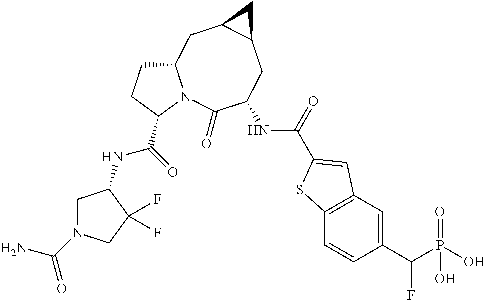 [SMILES: NC(=O)N1C[C@H](NC(=O)[C@@H]2CC[C@@H]3C[C@H]4C[C@H]4C[C@H](NC(=O)c4cc5cc(C(F)P(=O)(O)O)ccc5s4)C(=O)N32)C(F)(F)C1] | 658.3 | ¹H NMR (400 MHz, DEUTERIUM OXIDE) δ 7.98 (s, 3H), 7.58 (d, J = 8.8 Hz, 1H), 5.92-5.67 (m, 1H), 4.94 (t, J = 9.2 Hz, 1H), 4.64- 4.59 (m, 1H), 4.33- 4.24 (m, 1H), 4.03- 3.76 (m, 4H), 3.41- 3.29 (m, 1H), 2.47- 1.98 (m, 7H), 1.75- 1.59 (m, 1H), 1.16- 0.85 (m, 3H), 0.13 (d, J = 3.2 Hz, 1H) |
| | methyl) | | | |
| | phosphonic | | | |
| | acid | | | |
| 339 | (fluoro(2- (((3S,6S,7aS, 8aR,9aR)-3- (((3S,4S)-4- fluoro-1- (1H- imidazol-2- yl)pyrrolidin- 3-yl) carbamoyl)- 5-oxodeca- hydro-1H- cyclopropa[d] pyrrolo[1,2- a]azocin-6- yl)carbamoyl) benzo[b] thiophen-5- yl)methyl) phosphonic | 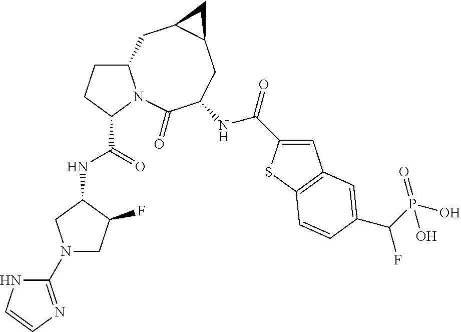 [SMILES: O=C(N[C@H]1C[C@@H]2C[C@@H]2C[C@H]2CC[C@@H](C(=O)N[C@H]3CN(c4ncc[nH]4)C[C@@H]3F)N2C1=O)c1cc2cc(C(F)P(=O)(O)O)ccc2s1] | 663.2 | ¹H NMR (400 MHz, DMSO-d₆) δ 8.88- 8.86 (m, 1H), 8.32 (d, J = 4.0 Hz, 1H), 8.23 (d, J = 4.0 Hz, 1H), 7.96-7.89 (m, 2H), 7.52 (t, J = 8.0 Hz, 1H), 6.97 (s, 2H), 5.73-5.52 (m, 1H), 5.27-5.07 (m, 1H), 4.94-4.81 (m, 1H), 4.46-4.38 (m, 1H), 4.32 (s, 1H), 4.18- 4.11 (m, 1H), 3.80- 3.69 (m, 2H), 2.37 (d, J = 2.4 Hz, 1H), 2.15- 2.09 (m, 2H), 2.01- 1.91 (m, 4H), 1.85- 1.79 (m, 1H), 1.67- 1.60 (m, 1H), 1.29- |
| | acid | | | 1.10 (m, 5H), 0.78- |
| | | | | 0.67 (m, 2H), 0.06- |
| | | | | −0.06 (m, 1H) |
| 337 | (fluoro(2- (((3S,6S,7aS, 8aR,9aR)-3- (3-(5- methyl- isoxazol-3- yl)azetidine- 1-carbonyl)- 5-oxodeca- hydro-1H- cyclopropa[d] pyrrolo[1,2- a]azocin-6- yl)carbamoyl) | 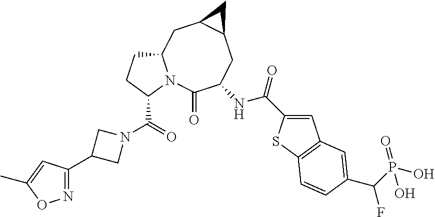 [SMILES: Cc1cc(C2CN(C(=O)[C@@H]3CC[C@@H]4C[C@H]5C[C@H]5C[C@H](NC(=O)c5cc6cc(C(F)P(=O)(O)O)ccc6s5)C(=O)N43)C2)no1] | 631.1 | ¹H NMR (400 MHz, CD₃OD) δ 8.11 (d, J = 2.4 Hz, 1H), 8.03 (s, 1H), 7.94 (d, J = 3.4, 8.2 Hz, 1H), 7.59 (d, J = 8.6 Hz, 1H), 6.40- 6.19 (m, 1H), 5.90- 5.74 (m, 1H), 5.06- 5.01 (m, 1H), 4.65 (d, J = 8.0 Hz, 1H), 4.54- 4.47 (m, 1H), 4.44- 4.25 (m, 3H), 4.11- 3.91 (m, 2H), 2.50- 2.45 (m, 1H), 2.40 (d, |
| | benzo[b] | | | J = 16.0 Hz, 3H), 2.33- |
| | thiophen-5- | | | 2.12 (m, 4H), 2.03- |
| | yl)methyl) | | | 1.92 (m, 2H), 1.79- |
| | phosphonic | | | 1.68 (m, 1H), 1.48- |
| | acid | | | 1.36 (m, 1H), 1.00- |
| | | | | 0.92 (m, 1H), 0.85- |
| | | | | 0.83 (m, 1H), 0.08- |
| | | | | 0.05 (m, 1H) |
| 336 | (fluoro(2- (((3S,6S,7aS, 8aR,9aR)-3- (3-(3- methoxy- isoxazol-5- yl)azetidine- 1-carbonyl)- 5-oxodeca- hydro-1H- cyclopropa[d] pyrrolo[1,2- a]azocin-6- | 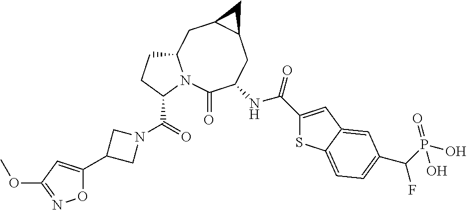 [SMILES: COc1cc(C2CN(C(=O)[C@@H]3CC[C@@H]4C[C@H]5C[C@H]5C[C@H](NC(=O)c5cc6cc(C(F)P(=O)(O)O)ccc6s5)C(=O)N43)C2)on1] | 647.1 | ¹H NMR (400 MHz, CD₃OD) δ 8.11 (d, J = 4.4 Hz, 1H), 8.03 (s, 1H), 7.94 (J = 2.8, 8.4 Hz, 1H), 7.59 (d, J = 8.6 Hz, 1H), 6.17- 6.06 (m, 1H), 5.91- 5.75 (m, 1H), 5.07- 5.00 (m, 1H), 4.73- 4.60 (m, 1H), 4.50 (d, J = 2.8, 8.6 Hz, 1H), 4.42-4.34 (m, 1H), 4.34-4.24 (m, 2H), |
| | yl)carbamoyl) | | | 4.10-3.96 (m, 2H), |
| | benzo[b] | | | 3.91 (d, J = 13.4 Hz, |
| | thiophen-5- | | | 3H), 2.50-2.40 (m, |
| | yl)methyl) | | | 1H), 2.34-2.09 (m, |
| | phosphonic | | | 4H), 2.03-1.90 (m, |
| | acid | | | 2H), 1.79-1.67 (m, |
| | | | | 1H), 1.48-1.35 (m, |
| | | | | 1H), 1.00-0.90 (m, |
| | | | | 1H), 0.88-0.80 (m, |
| | | | | 1H), 0.10-0.04 (m, |
| | | | | 1H) |
| 335 | ((2- (((3S,6S,7aS, 8aR,9aR)-3- (3-((1- methyl-1H- pyrazol-4- yl)oxy) azetidine-1- carbonyl)-5- oxodecahydro- 1H- | 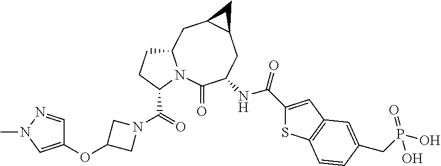 [SMILES: Cn1cc(OC2CN(C(=O)[C@@H]3CC[C@@H]4C[C@H]5C[C@H]5C[C@H](NC(=O)c5cc6cc(CP(=O)(O)O)ccc6s5)C(=O)N43)C2)cn1] | 628.2 | ¹H NMR (400 MHz, CD₃OD) δ = 8.01 (d, J = 17.6 Hz, 1H), 7.82 (m, 2H), 7.41 (m, 1H), 7.30 (d, J = 14.8 Hz, 1H), 7.17 (d, J = 12.0 Hz, 1H), 5.07- 4.99 (m, 2H), 4.84- 4.80 (m, 1H), 4.57 (d, J = 5.2 Hz, 1H), 4.51 (m, 1H), 4.37-4.18 |
| | cyclopropa[d] | | | (m, 3H), 3.75 (d, J = |
| | pyrrolo[1,2- | | | 19.6 Hz, 3H), 3.27 (d, |
| | a]azocin-6- | | | J = 2.0 Hz, 1H), 3.22 |
| | yl)carbamoyl) | | | (d, J = 2.0 Hz, 1H), |
| | benzo[b] | | | 2.50-2.40 (m, 1H), |
| | thiophen-5- | | | 2.35-2.24 (m, 2H), |
| | yl)methyl) | | | 2.21 (m, 1H), 2.17- |
| | phosphonic | | | 2.08 (m, 1H), 1.96 |
| | acid | | | (m, 2H), 1.79-1.67 |
| | | | | (m, 1H), 1.46-1.36 |
| | | | | (m, 1H), 0.99-0.90 |
| | | | | (m, 1H), 0.88-0.80 |
| | | | | (m, 1H), 0.10-0.01 |
| | | | | (m, 1H) |
| 334 | ((2- (((3S,6S,7aS, 8aR,9aR)-5- oxo-3-(3- (pyrazin-2- yloxy) azetidine-1- carbonyl) decahydro-1H- cyclopropa[d] pyrrolo[1,2- a]azocin-6- yl)carbamoyl) benzo[b] | 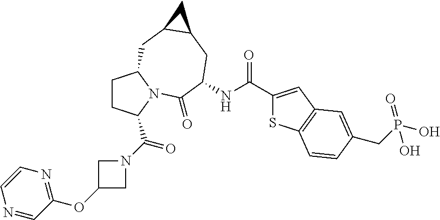 [SMILES: O=C(N[C@H]1C[C@@H]2C[C@@H]2C[C@H]2CC[C@@H](C(=O)N3CC(Oc4cnccn4)C3)N2C1=O)c1cc2cc(CP(=O)(O)O)ccc2s1] | 626.3 | ¹H NMR (400 MHz, CD₃OD) δ 8.32-8.26 (m, 1H), 8.20-8.08 (m, 2H), 8.02 (d, J = 19.2 Hz, 1H), 7.87- 7.78 (m, 2H), 7.42 (t, J = 8.4 Hz, 1H), 5.51- 5.40 (m, 1H), 5.08- 4.94 (m, 2H), 4.75- 4.60 (m, 1H), 4.54- 4.50 (m, 1H), 4.49- 4.34 (m, 1H), 4.34- 4.24 (m, 1H), 4.09- 3.95 (m, 1H), 3.28- |
| | thiophen-5- | | | 3.19 (m, 2H), 2.55- |
| | yl)methyl) | | | 2.38 (m, 1H), 2.34- |
| | phosphonic | | | 2.09 (m, 4H), 2.03- |
| | acid | | | 1.90 (m, 2H), 1.80- |
| | | | | 1.67 (m, 1H), 1.47- |
| | | | | 1.34 (m, 1H), 1.01- |
| | | | | 0.90 (m, 1H), 0.88- |
| | | | | 0.79 (m, 1H), 0.10- |
| | | | | 0.00 (m, 1H) |
| 333 | (fluoro(2- (((3S,6S,7aS, 8aR,9aR)-3- (3-((methyl- sulfonyl) methyl) azetidine-1- carbonyl)-5- oxodecahydro- 1H- cyclopropa[d] pyrrolo[1,2- a]azocin-6- yl)carbamoyl) benzo[b] thiophen-5- yl)methyl) | 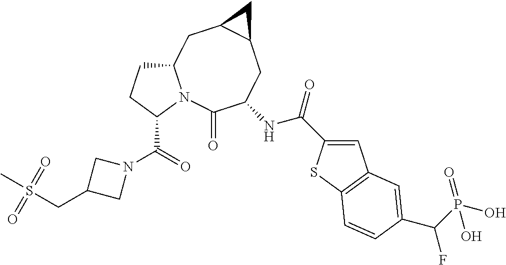 [SMILES: CS(=O)(=O)CC1CN(C(=O)[C@@H]2CC[C@@H]3C[C@H]4C[C@H]4C[C@H](NC(=O)c4cc5cc(C(F)P(=O)(O)O)ccc5s4)C(=O)N32)C1] | 642.3 | ¹H NMR (400 MHz, CD₃OD) δ 8.11 (d, J = 2.0 Hz, 1H), 8.02 (s, 1H), 7.90 (s, 1H), 7.58 (d, J = 8.4 Hz, 1H), 5.89-5.72 (m, 1H), 5.04-5.00 (m, 1H), 4.80-4.71 (m, 1H), 4.48-4.41 (m, 2H), 4.31-4.24 (m, 1H), 4.17-4.12 (m, 1H), 3.92-3.80 (m, 1H), 3.57-3.51 (m, 2H), 3.27-3.16 (m, 1H), 2.96 (d, J = 3.6 Hz, 3H), 2.44 (td, J = 7.4, 15.0 Hz, 1H), |
| | phosphonic | | | 2.32-2.26 (m, 1H), |
| | acid | | | 2.22-2.11 (m, 2H), |
| | | | | 2.02-1.90 (m, 2H), |
| | | | | 1.72 (ddd, J = 7.4, |
| | | | | 12.2, 15.2 Hz, 1H), |
| | | | | 1.50-1.22 (m, 2H), |
| | | | | 0.99-0.79 (m, 2H), |
| | | | | 0.05-0.34 (m, 1H) |
| 331 | (fluoro(2- (((3S,6S,7aR, 8aS,9aR)-3- (3-(4-methyl- 4H-1,2,4- triazol-3- yl)azetidine- 1-carbonyl)- 5-oxodeca- hydro-1H- cyclopropa[d] pyrrolo[1,2- a]azocin-6- yl)carbamoyl) benzo[b] thiophen-5- | 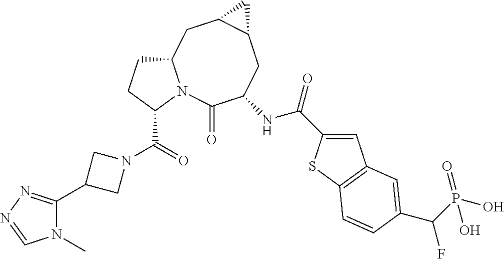 [SMILES: Cn1cnnc1C1CN(C(=O)[C@@H]2CC[C@@H]3C[C@@H]4C[C@@H]4C[C@H](NC(=O)c4cc5cc(C(F)P(=O)(O)O)ccc5s4)C(=O)N32)C1] | 631.4 | ¹H NMR (400 MHz, DMSO-d₆) δ 8.89- 8.75 (m, 1H), 8.64- 8.48 (m, 1H), 8.32- 8.18 (m, 1H), 8.04 (t, J = 8.0 Hz, 1H), 7.98 (s, 1H), 7.60-7.43 (m, 1H), 5.95-5.71 (m, 1H), 4.84-4.74 (m, 1H), 4.65-4.52 (m, 1H), 4.50-4.39 (m, 1H), 4.37-4.21 (m, 2H), 4.20-4.12 (m, 2H), 4.09-4.04 (m, 1H), 3.55 (s, 3H), 2.27-2.13 (m, 2H), |
| | yl)methyl) | | | 2.10-1.97 (m, 2H), |
| | phosphonic | | | 1.94-1.81 (m, 1H), |
| | acid | | | 1.74-1.61 (m, 1H), |
| | | | | 1.57-1.47 (m, 1H), |
| | | | | 1.38-1.28 (m, 1H), |
| | | | | 1.28-1.18 (m, 2H), |
| | | | | 0.76 (d, J = 4.0 Hz, |
| | | | | 1H), 0.10 (s, 1H) |
| 330 | ((2- (((3S,6S,7aS, 8aR,9aR)-3- (((3S,4S)-1- carbamoyl-4- fluoro- pyrrolidin-3- yl) carbamoyl)-5- oxodecahydro- 1H- cyclopropa[d] pyrrolo[1,2- a]azocin-6- yl)carbamoyl) benzo[b] thiophen-5- yl)fluoro- methyl) | 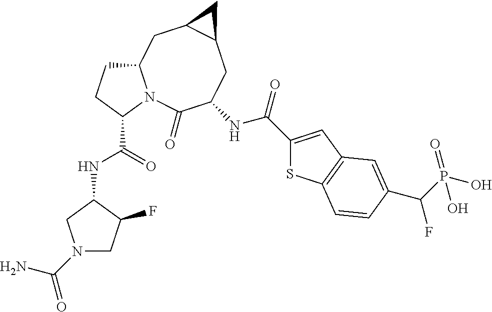 [SMILES: NC(=O)N1C[C@H](NC(=O)[C@@H]2CC[C@@H]3C[C@H]4C[C@H]4C[C@H](NC(=O)c4cc5cc(C(F)P(=O)(O)O)ccc5s4)C(=O)N32)[C@@H](F)C1] | 640.1 | ¹H NMR (400 MHz, DMSO-d₆) δ 8.88 (d, J = 7.2 Hz, 1H), 8.27 (s, 1H), 8.16 (d, J = 6.8 Hz, 1H), 8.07- 7.92 (m, 2H), 7.52 (d, J = 8.0 Hz, 1H), 5.99- 5.71 (m, 3H), 5.08- 4.82 (m, 2H), 4.40 (d, J = 4.8 Hz, 1H), 4.28- 4.21 (m, 1H), 4.17- 4.11 (m, 1H), 3.52 (s, 1H), 3.48 (d, J = 4.8 Hz, 1H), 3.46-3.43 (m, 2H), 3.19 (d, J = 11.2 Hz, 2H), 2.37 (d, J = 8.0 Hz, 1H), 2.17- 2.07 (m, 2H), 2.01- 1.80 (m, 4H), 1.70- |
| | phosphonic | | | 1.58 (m, 1H), 1.26- |
| | acid | | | 1.05 (m, 1H), 0.87- |
| | | | | 0.66 (m, 2H), 0.03 (d, |
| | | | | J = 4.8 Hz, 1H) |
| 328 | (fluoro(2- (((3S,6S,7aS, 8aR,9aR)-3- (3-(3- methyl- isoxazol-5- yl)azetidine- 1-carbonyl)- 5-oxodeca- hydro-1H- cyclopropa[d] pyrrolo[1,2- a]azocin-6- | 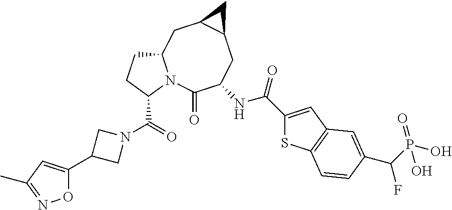 [SMILES: Cc1cc(C2CN(C(=O)[C@@H]3CC[C@@H]4C[C@H]5C[C@H]5C[C@H](NC(=O)c5cc6cc(C(F)P(=O)(O)O)ccc6s5)C(=O)N43)C2)on1] | 631.1 | ¹H NMR (400 MHz, CD₃OD) δ 8.11 (d, J = 4.2 Hz, 1H), 8.02 (s, 1H), 7.94 (d, J = 3.8, 8.2 Hz, 1H), 7.58 (d, J = 8.8 Hz, 1H), 6.35- 6.23 (m, 1H), 5.91- 5.74 (m, 1H), 5.05- 5.01 (m, 1H), 4.69- 4.64 (m, 1H), 4.51 (d, J = 5.8 Hz, 1H), 4.42- 4.34 (m, 1H), 4.33- 4.23 (m, 2H), 4.12- |
| | yl)carbamoyl) | | | 4.00 (m, 2H), 2.51- |
| | benzo[b] | | | 2.39 (m, 1H), 2.36- |
| | thiophen-5- | | | 2.29 (m, 1H), 2.25 (d, |
| | yl)methyl) | | | J = 14.0 Hz, 3H), 2.21- |
| | phosphonic | | | 2.11 (m, 2H), 2.05- |
| | acid | | | 1.88 (m, 3H), 1.79- |
| | | | | 1.68 (m, 1H), 1.48- |
| | | | | 1.33 (m, 1H), 1.02- |
| | | | | 0.90 (m, 1H), 0.89- |
| | | | | 0.79 (m, 1H), 0.14- |
| | | | | 0.00 (m, 1H) |
| 327 | (fluoro(2- (((3S,6S,7aS, 8aR,9aR)-3- (3-(5-methyl- 1,3,4- oxadiazol-2- yl)azetidine- 1-carbonyl)- 5-oxodeca- hydro-1H- cyclopropa[d] pyrrolo[1,2- a]azocin-6- yl)carbamoyl) | 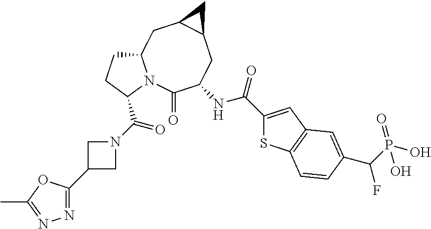 [SMILES: Cc1nnc(C2CN(C(=O)[C@@H]3CC[C@@H]4C[C@H]5C[C@H]5C[C@H](NC(=O)c5cc6cc(C(F)P(=O)(O)O)ccc6s5)C(=O)N43)C2)o1] | 632.3 | ¹H NMR (400 MHz, CD₃OD) δ 8.11-7.99 (m, 2H), 7.84 (t, J = 8.4 Hz, 1H), 7.64 (d, J = 7.4 Hz, 1H), 5.73- 5.54 (m, 1H), 5.01 (q, J = 8.8 Hz, 2H), 4.67 (t, J = 8.8 Hz, 1H), 4.54-4.42 (m, 2H), 4.37-4.24 (m, 2H), 4.19-4.13 (m, 1H), 2.59-2.48 (m, 3H), 2.48-2.40 (m, 1H), 2.38-2.06 (m, 4H), |
| | benzo[b] | | | 2.04-1.90 (m, 2H), |
| | thiophen-5- | | | 1.80-1.67 (m, 1H), |
| | yl)methyl) | | | 1.40 (d, J = 6.0 Hz, |
| | phosphonic | | | 1H), 1.01-0.89 (m, |
| | acid | | | 1H), 0.84 (d, J = 3.2, |
| | | | | 7.4 Hz, 1H), 0.07 (d, |
| | | | | J = 4.4 Hz, 1H) |
| 326 | (fluoro(2- (((3S,6S,7aS, 8aR,9aR)-3- (3-(3-methyl- 1,2,4- oxadiazol-5- yl)azetidine- 1-carbonyl)- 5-oxodeca- hydro-1H- cyclopropa[d] pyrrolo[1,2- a]azocin-6- yl)carbamoyl) | 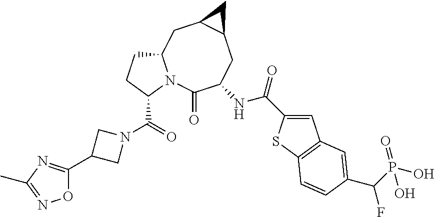 [SMILES: Cc1noc(C2CN(C(=O)[C@@H]3CC[C@@H]4C[C@H]5C[C@H]5C[C@H](NC(=O)c5cc6cc(C(F)P(=O)(O)O)ccc6s5)C(=O)N43)C2)n1] | 632.2 | ¹H NMR (400 MHz, CD₃OD) δ 8.07 (d, J = 19.0 Hz, 1H), 8.00 (d, J = 12.8 Hz, 1H), 7.92 (d, J = 8.6, 12 Hz, 1H), 7.61-7.54 (m, 1H), 5.95-5.73 (m, 1H), 5.09-4.95 (m, 2H), 4.85-4.65 (m, 1H), 4.56-4.41 (m, 2H), 4.40-4.25 (m, 2H), 4.21-4.13 (m, 1H), 2.51-2.40 (m, 1H), 2.39-2.32 (m, |
| | benzo[b] | | | 3H), 2.30-2.08 (m, |
| | thiophen-5- | | | 4H), 2.04-1.90 (m, |
| | yl)methyl) | | | 2H), 1.79-1.67 (m, |
| | phosphonic | | | 1H), 1.47-1.34 (m, |
| | acid | | | 1H), 1.00-0.89 (m, |
| | | | | 1H), 0.84 (t, J = 4.0, |
| | | | | 7.8 Hz, 1H), 0.06 (m, |
| | | | | J = 4.6 Hz, 1H) |
| 324 | (fluoro(2- (((3S,6S,7aS, 8aR,9aR)-3- (3-(5- methyloxazol- 2- yl)azetidine- 1-carbonyl)- 5-oxodeca- hydro-1H- cyclopropa[d] pyrrolo[1,2- a]azocin-6- yl)carbamoyl) | 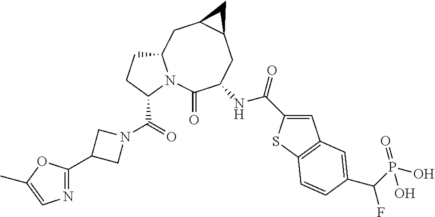 [SMILES: Cc1cnc(C2CN(C(=O)[C@@H]3CC[C@@H]4C[C@H]5C[C@H]5C[C@H](NC(=O)c5cc6cc(C(F)P(=O)(O)O)ccc6s5)C(=O)N43)C2)o1] | 631.2 | ¹H NMR (400 MHz, CD₃OD) δ 8.11- 8.03 (m, 1H), 8.02- 7.96 (m, 1H), 7.95- 7.87 (m, 1H), 7.58 (m, 1H), 6.80-6.68 (m, 1H), 5.93-5.73 (m, 1H), 5.07-4.99 (m, 1H), 4.97-4.92 (m, 1H), 4.80-4.60 (m, 1H), 4.55-4.49 (m, 1H), 4.36 (s, 1H), 4.33-4.23 (m, 2H), 4.14-3.98 (m, 1H), |
| | benzo[b] | | | 2.49-2.41 (m, 1H), |
| | thiophen-5- | | | 2.35-2.09 (m, 7H), |
| | yl)methyl) | | | 2.03-1.94 (m, 2H), |
| | phosphonic | | | 1.79-1.67 (m, 1H), |
| | acid | | | 1.47-1.36 (m, 1H), |
| | | | | 1.00-0.89 (m, 1H), |
| | | | | 0.88-0.79 (m, 1H), |
| | | | | 0.14-0.00 (m, 1H) |
| 322 | (fluoro(2- (((3S,6S,7aS, 8aR,9aR)-3- (3-(oxazol-2- yl)azetidine- 1-carbonyl)- 5-oxodeca- hydro-1H- cyclopropa[d] pyrrolo[1,2- a]azocin-6- yl)carbamoyl) benzo[b] thiophen-5- yl)methyl) phosphonic acid | 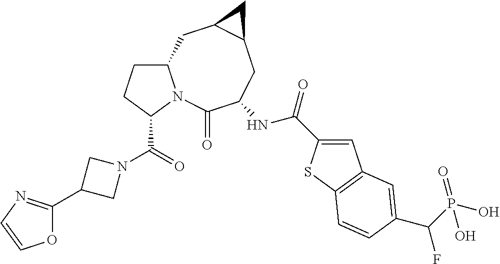 [SMILES: O=C(N[C@H]1C[C@@H]2C[C@@H]2C[C@H]2CC[C@@H](C(=O)N3CC(c4ncco4)C3)N2C1=O)c1cc2cc(C(F)P(=O)(O)O)ccc2s1] | 617.2 | ¹H NMR (400 MHz, CD₃OD) δ 8.12-8.03 (m, 1H), 8.03-7.96 (m, 1H), 7.95-7.79 (m, 2H), 7.58 (d, J = 7.6 Hz, 1H), 7.21- 7.05 (m, 1H), 5.93- 5.72 (m, 1H), 5.04 (t, J = 8.8 Hz, 1H), 4.98- 4.92 (m, 1H), 4.83- 4.60 (m, 1H), 4.55- 4.38 (m, 2H), 4.36- 4.20 (m, 2H), 4.17- 4.06 (m, 1H), 2.45 (m, 1H), 2.35-2.22 (m, 2H), 2.22-2.07 (m, 2H), 2.04-1.88 |
| | | | | (m, 2H), 1.79-1.66 |
| | | | | (m, 1H), 1.46-1.34 |
| | | | | (m, 1H), 0.99-0.89 |
| | | | | (m, 1H), 0.84 (m, |
| | | | | 1H), 0.15-0.02 (m, |
| | | | | 1H |
| 320 | (fluoro(2- (((3S,6S,7aR, 8aS,9aR)-3- (3-(4- methyl- pyridin-3- yl)azetidine- 1-carbonyl)- 5-oxodeca- hydro-1H- cyclopropa[d] pyrrolo[1,2- a]azocin-6- yl)carbamoyl) benzo[b] thiophen-5- yl)methyl) | 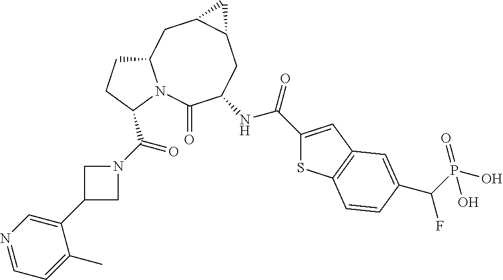 [SMILES: Cc1ccncc1C1CN(C(=O)[C@@H]2CC[C@@H]3C[C@@H]4C[C@@H]4C[C@H](NC(=O)c4cc5cc(C(F)P(=O)(O)O)ccc5s4)C(=O)N32)C1] | 641.2 | ¹H NMR (400 MHz, DMSO-d₆) δ 8.84- 8.75 (m, 1H), 8.74- 8.59 (m, 1H), 8.54- 8.44 (m, 1H), 8.29- 8.19 (m, 1H), 8.08- 7.93 (m, 2H), 7.59- 7.45 (m, 2H), 5.91- 5.74 (m, 1H), 4.86- 4.77 (m, 1H), 4.67- 4.44 (m, 1H), 4.40- 4.28 (m, 2H), 4.26- 4.12 (m, 3H), 4.06- 4.00 (m, 1H), 2.35- 2.29 (m, 3H), 2.28- 2.15 (m, 2H), 2.12- 1.98 (m, 2H), 1.94- |
| | phosphonic | | | 1.79 (m, 2H), 1.76- |
| | acid | | | 1.62 (m, 1H), 1.59- |
| | | | | 1.46 (m, 1H), 1.39- |
| | | | | 1.28 (m, 1H), 1.27- |
| | | | | 1.17 (m, 1H), 0.82- |
| | | | | 0.71 (m, 1H), 0.12- |
| | | | | 0.00 (m, 1H) |
| 318 | (fluoro(2- (((3S,6S,7aR, 8aS,9aR)-3- (3-(4- methoxy- pyridin-3- yl)azetidine- 1-carbonyl)- 5-oxodeca- hydro-1H- cyclopropa[d] pyrrolo[1,2- a]azocin-6- yl)carbamoyl) benzo[b] thiophen-5- yl)methyl) | 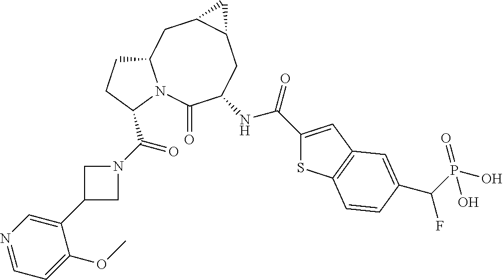 [SMILES: COc1ccncc1C1CN(C(=O)[C@@H]2CC[C@@H]3C[C@@H]4C[C@@H]4C[C@H](NC(=O)c4cc5cc(C(F)P(=O)(O)O)ccc5s4)C(=O)N32)C1] | 657.2 | ¹H NMR (400 MHz, CD₃OD) 8.14-8.03 (m, 2H), 8.02-7.88 (m, 1H), 7.84-7.78 (m, 1H), 7.77-7.69 (m, 1H), 7.65-7.50 (m, 1H), 7.19-6.92 (m, 1H), 5.80-5.54 (m, 1H), 4.65-4.47 (m, 2H), 4.39 (dd, J = 6.0, 17.2 Hz, 1H), 4.34-4.23 (m, 2H), 4.22-4.15 (m, 1H), 4.09-4.01 (m, 1H), 3.99 (s, 2H), 3.95- 3.87 (m, 2H), 2.41- 2.24 (m, 2H), 2.21- |
| | phosphonic | | | 2.08 (m, 2H), 2.04- |
| | acid | | | 1.93 (m, 2H), 1.86- |
| | | | | 1.74 (m, 1H), 1.46- |
| | | | | 1.36 (m, 1H), 1.35- |
| | | | | 1.17 (m, 2H), 0.92- |
| | | | | 0.79 (m, 1H), 0.16 (q, |
| | | | | J = 4.8 Hz, 1H) |
| 317 | (fluoro(2- (((3S,6S,7aR, 8aS,9aR)-5- oxo-3-(3- (pyrimidin-2- yl)azetidine- 1- carbonyl) decahydro-1H- cyclopropa[d] pyrrolo[1,2- a]azocin-6- yl)carbamoyl) benzo[b] thiophen-5- yl)methyl) phosphonic | 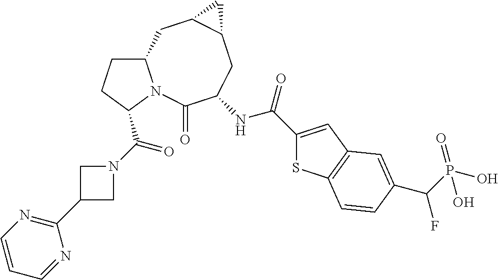 [SMILES: O=C(N[C@H]1C[C@H]2C[C@H]2C[C@H]2CC[C@@H](C(=O)N3CC(c4ncccn4)C3)N2C1=O)c1cc2cc(C(F)P(=O)(O)O)ccc2s1] | 628.2 | ¹H NMR (400 MHz, CD₃OD) δ 8.78-8.81 (m, 1H), 8.70-8.82 (m, 1H), 7.86-8.16 (m, 3H), 7.53-7.65 (m, 1H), 7.28-7.44 (m, 1H), 5.69-5.88 (m, 1H), 4.94 (d, J = 6.00 Hz, 2H), 4.79 (s, 1H), 4.54 (dd, J = 8.40, 5.60 Hz, 1H), 4.40-4.49 (m, 2H), 4.28-4.38 (m, 1H), 4.11-4.25 (m, 1H), 2.10-2.39 (m, 3H), 1.92-2.06 (m, 2H), 1.79-1.91 (m, 1H), |
| | acid | | | 1.64-1.78 (m, 1H), |
| | | | | 1.46 (s, 1H), 1.37 (dd, |
| | | | | J = 6.40, 2.80 Hz, 1H), |
| | | | | 1.22-1.31 (m, 1H), |
| | | | | 0.85 (dt, J = 8.00, 4.00 |
| | | | | Hz, 1H), 0.16-0.22 |
| | | | | (m, 1H) |
| 313 | (fluoro(2- (((3S,6S,7aR, 8aS,9aR)-3- ((R)-3-(4- fluoropyridin- 3-yl) pyrrolidine-1- carbonyl)-5- oxodecahydro- 1H- cyclopropa[d] pyrrolo[1,2- | 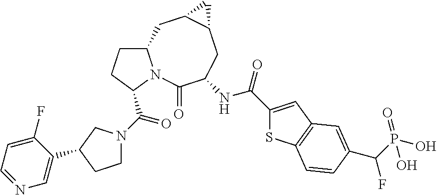 [SMILES: O=C(N[C@H]1C[C@H]2C[C@H]2C[C@H]2CC[C@@H](C(=O)N3CC[C@H](c4cnccc4F)C3)N2C1=O)c1cc2cc(C(F)P(=O)(O)O)ccc2s1] | 659 | ¹H NMR (400 MHz, DMSO-d₆) δ 8.79 (d, J = 7.6 Hz, 1H), 8.70- 8.50 (m, 2H), 8.26 (s, 1H), 8.11-7.94 (m, 2H), 7.53 (d, J = 8.0 Hz, 1H), 7.38 (d, J = 5.6 Hz, 1H), 5.95- 5.72 (m, 1H), 4.83 (s, 1H), 4.60-4.53 (m, 1H), 4.23-4.14 (m, 2H), 3.91-3.76 |
| | a]azocin-6- | | | (m, 2H), 3.67-3.54 |
| | yl)carbamoyl) | | | (m, 3H), 3.35 (d, J = |
| | benzo[b] | | | 8.8 Hz, 1H), 2.39- |
| | thiophen-5- | | | 2.21 (m, 3H), 2.16- |
| | yl)methyl) | | | 1.93 (m, 4H), 1.92- |
| | phosphonic | | | 1.81 (m, 1H), 1.73- |
| | acid | | | 1.56 (m, 2H), 1.41- |
| | | | | 1.13 (m, 2H), 0.77 (d, |
| | | | | J = 4.8 Hz, 1H), 0.06 |
| | | | | (d, J = 4.8 Hz, 1H) |
| 312 | (fluoro(2- (((3S,6S,7aS, 8aR,9aR)-3- (3-(isoxazol- 3- yl)azetidine- 1-carbonyl)- 5-oxodeca- hydro-1H- cyclopropa[d] pyrrolo[1,2- a]azocin-6- yl)carbamoyl) benzo[b] thiophen-5- yl)methyl) phosphonic | 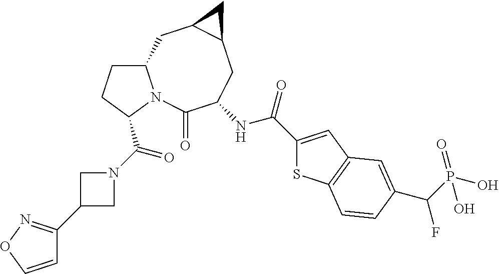 [SMILES: O=C(N[C@H]1C[C@@H]2C[C@@H]2C[C@H]2CC[C@@H](C(=O)N3CC(c4ccon4)C3)N2C1=O)c1cc2cc(C(F)P(=O)(O)O)ccc2s1] | 617.1 | ¹H NMR (400 MHz, DMSO-d₆) δ 8.95- 8.82 (m, 2H), 8.32- 8.25 (m, 1H), 8.07- 7.96 (m, 2H), 7.59- 7.51 (m, 1H), 6.76- 6.62 (m, 1H), 5.91- 5.76 (m, 1H), 4.97- 4.85 (m, 2H), 4.62- 4.51 (m, 2H), 4.35 (dd, J = 3.6, 7.6 Hz, 1H), 4.26 (s, 1H), 4.18 (d, J = 8.8 Hz, 1H), 3.97 (d, J = 7.6 Hz, 1H), 3.87 (dd, J = 5.6, 9.4 Hz, 1H), 2.38- 2.28 (m, 1H), 2.17- |
| | acid | | | 2.08 (m, 2H), 2.08- |
| | | | | 1.94 (m, 2H), 1.91- |
| | | | | 1.80 (m, 2H), 1.69- |
| | | | | 1.59 (m, 1H), 1.42- |
| | | | | 1.28 (m, 1H), 0.86- |
| | | | | 0.76 (m, 1H), 0.72 |
| | | | | (dd, J = 4.0, 7.6 Hz, |
| | | | | 1H), 0.04-−0.04 (m, |
| | | | | 1H) |
| 305 | (fluoro(2- (((3S,6S,7aS, 8aR,9aR)-5- oxo-3-(3- (pyrimidin-2- yl)azetidine- 1-carbonyl) decahydro- 1H- cyclopropa[d] pyrrolo[1,2- a]azocin-6- yl)carbamoyl) benzo[b] thiophen-5- yl)methyl) phosphonic acid | 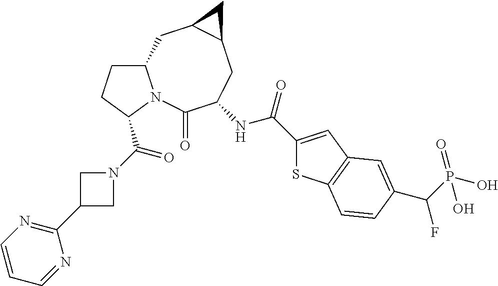 [SMILES: O=C(N[C@H]1C[C@@H]2C[C@@H]2C[C@H]2CC[C@@H](C(=O)N3CC(c4ncccn4)C3)N2C1=O)c1cc2cc(C(F)P(=O)(O)O)ccc2s1] | 628.2 | ¹H NMR (400 MHz, CD₃OD) δ 8.79 (d, J = 4.8 Hz, 1H), 8.76- 8.66 (m, 1H), 8.13- 7.97 (m, 2H), 7.96- 7.84 (m, 1H), 7.62- 7.53 (m, 1H), 7.38 (t, J = 4.8 Hz, 1H), 5.91- 5.73 (m, 1H), 5.08- 4.99 (m, 2H), 4.70- 4.51 (m, 2H), 4.47- 4.37 (m, 1H), 4.36- 4.27 (m, 1H), 4.26- 4.07 (m, 2H), 2.51- 2.37 (m, 1H), 2.35- 2.26 (m, 2H), 2.24- 2.11 (m, 2H), 2.06- 1.88 (m, 2H), 1.80- |
| | | | | 1.67 (m, 1H), 1.51- |
| | | | | 1.38 (m, 1H), 1.03- |
| | | | | 0.79 (m, 2H), 0.10- |
| | | | | 0.00 (m, 1H) |
| 304 | (fluoro(2- (((3S,6S,7aS, 8aR,9aR)-3- (3-(4-methyl- 4H-1,2,4- triazol-3- yl)azetidine- 1-carbonyl)- 5-oxodeca- hydro-1H- cyclopropa[d] pyrrolo[1,2- a]azocin-6- yl)carbamoyl) benzo[b] thiophen-5- yl)methyl) | 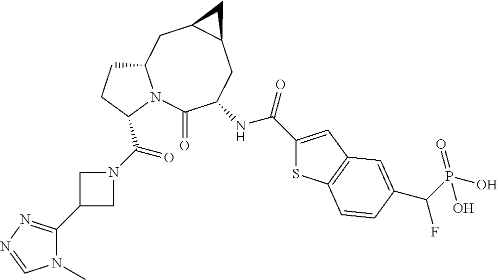 [SMILES: Cn1cnnc1C1CN(C(=O)[C@@H]2CC[C@@H]3C[C@H]4C[C@H]4C[C@H](NC(=O)c4cc5cc(C(F)P(=O)(O)O)ccc5s4)C(=O)N32)C1] | 631.4 | ¹H NMR (400 MHz, CD₃OD) δ 8.60-8.51 (m, 1H), 8.03-7.85 (m, 3H), 7.83-7.52 (m, 1H), 5.75-5.63 (m, 1H), 4.98-4.92 (m, 1H), 4.68-4.66 (m, 1H), 4.52-4.42 (m, 2H), 4.40-4.29 (m, 1H), 4.26-4.03 (m, 3H), 3.62-3.56 (m, 3H), 2.44-2.15 (m, 1H), 2.14-2.13 (m, 2H), 2.10-1.96 (m, 2H), 1.95-1.83 (m, 2H), 1.91-1.68 (m, 1H), 1.66 (dd, J = |
| | phosphonic | | | 4.8, 7.6 Hz, 1H), 0.88- |
| | acid | | | 0.86 (m, 1H), 0.86- |
| | | | | 0.75 (m, 1H), 0.02- |
| | | | | −0.01 (m, 1H) |
| 303 | (difluoro(2- (((3S,6S,7aS, 8aR,9aR)-3- (3-(4- fluoropyridin- 3- yl)azetidine- 1-carbonyl)- 5-oxodeca- hydro-1H- cyclopropa[d] pyrrolo[1,2- a]azocin-6- yl)carbamoyl) benzo[b] thiophen-5- yl)methyl) phosphonic | 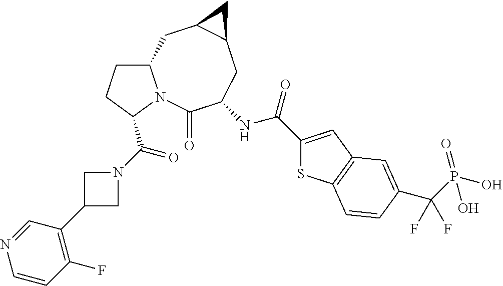 [SMILES: O=C(N[C@H]1C[C@@H]2C[C@@H]2C[C@H]2CC[C@@H](C(=O)N3CC(c4cnccc4F)C3)N2C1=O)c1cc2cc(C(F)(F)P(=O)(O)O)ccc2s1] | 662.9 | ¹H NMR (400 MHz, DMSO-d₆) δ 8.96- 8.82 (m, 1H), 8.67 (d, J = 10.4 Hz, 1H), 8.55- 8.48 (m, 2H), 7.95 (d, J = 8.4 Hz, 1H), 7.61 (d, J = 8.4 Hz, 1H), 7.35-7.28 (m, 1H), 4.96-4.87 (m, 2H), 4.67-4.59 (m, 1H), 4.40-4.37 (m, 1H), 4.35-4.23 (m, 1H), 4.23-4.09 (m, 2H), 4.08-3.94 (m, 1H), 2.18-2.14 (m, 5H), 2.18-2.05 (m, 3H), 1.41-1.30 (m, 1H), 0.85-0.67 (m, |
| | acid | | | 2H), 0.00-0.04 (s, |
| | | | | 1H) |
| 302 | (2- (((3S,6S,7aR, 8aS,9aR)-3- (3-(1,1- dioxidothio- morpholino) azetidine-1- carbonyl)-5- oxodecahydro- 1H- cyclopropa[d] pyrrolo[1,2- a]azocin-6- yl)carbamoyl) benzo[b] thiophen-5- yl)fluoro- | 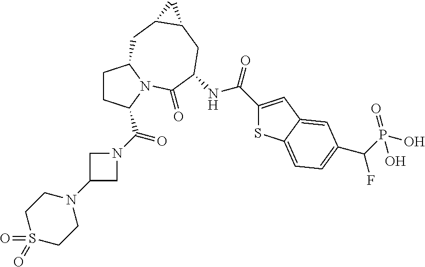 [SMILES: O=C(N[C@H]1C[C@H]2C[C@H]2C[C@H]2CC[C@@H](C(=O)N3CC(N4CCS(=O)(=O)CC4)C3)N2C1=O)c1cc2cc(C(F)P(=O)(O)O)ccc2s1] | 683.3 | ¹H NMR (400 MHz, CD₃OD) δ 8.11-8.01 (m, 2H), 7.89 (d, J = 8.4 Hz, 1H), 7.63 (d, J = 7.6 Hz, 1H), 5.78- 5.58 (m, 1H), 4.94 (t, J = 5.2 Hz, 1H), 4.59- 4.36 (m, 2H), 4.36- 4.26 (m, 2H), 4.14- 4.05 (m, 1H), 3.92- 3.75 (m, 1H), 3.54- 3.41 (m, 1H), 3.18- 3.07 (m, 4H), 2.90 (s, 4H), 2.35 (d, J = 14.8 Hz, 1H), 2.29-2.08 (m, 3H), 2.06-1.91 (m, 2H), 1.89-1.77 |
| | methyl) | | | (m, 1H), 1.75-1.62 |
| | phosphonic | | | (m, 1H), 1.52-1.40 |
| | acid | | | (m, 1H), 1.33-1.21 |
| | | | | (m, 1H), 0.87-0.82 |
| | | | | (m, 1H), 0.17 (q, J = |
| | | | | 5.2 Hz, 1H) |
| 301 | ((2- (((3S,6S,7aS, 8aR,9aR)-3- (3-(1,1- dioxidothio- morpholino) azetidine-1- carbonyl)-5- oxodecahydro- 1H- cyclopropa[d] pyrrolo[1,2- a]azocin-6- yl)carbamoyl) | 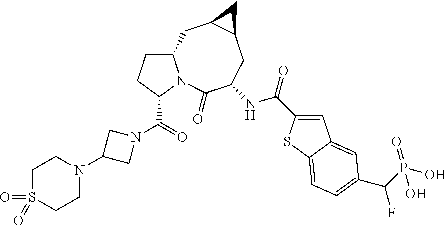 [SMILES: O=C(N[C@H]1C[C@@H]2C[C@@H]2C[C@H]2CC[C@@H](C(=O)N3CC(N4CCS(=O)(=O)CC4)C3)N2C1=O)c1cc2cc(C(F)P(=O)(O)O)ccc2s1] | 683.1 | ¹H NMR (400 MHz, DMSO-d₆) δ 8.85- 8.82 (m, 1H), 8.29 (s, 1H), 8.07-7.95 (m, 2H), 7.53 (d, J = 8.4 Hz, 1H), 5.93-5.75 (m, 1H), 4.97-4.87 (m, 1H), 4.54-4.50 (m, 1H), 4.34-4.29 (m, 1H), 4.28-4.10 (m, 3H), 4.04-4.01 (m, 1H), 3.92-3.79 (m, 1H), 3.70-3.61 (m, 1H), 3.42 (d, J = |
| | benzo[b] | | | 4.0 Hz, 1H), 3.12 (d, |
| | thiophen-5- | | | J = 4.0 Hz, 3H), 2.80 |
| | yl)fluoro- | | | (d, J = 15.6 Hz, 3H), |
| | methyl) | | | 2.35-3.29 (m, 1H), |
| | phosphonic | | | 2.17-1.94 (m, 4H), |
| | acid | | | 1.90-1.76 (m, 2H), |
| | | | | 1.68-1.57 (m, 1H), |
| | | | | 1.41-1.21 (m, 1H), |
| | | | | 0.84-0.67 (m, 2H), |
| | | | | −0.01 (d, J = 4.4 Hz, |
| | | | | 1H) |
| 300 | (difluoro(2- (((3S,6S,7aS, 8aR,9aR)-3- (3-(oxazol-2- yl)azetidine- 1-carbonyl)- 5-oxodeca- hydro-1H- cyclopropa[d] pyrrolo[1,2- a]azocin-6- yl)carbamoyl) benzo[b] thiophen-5- yl)methyl) phosphonic acid | 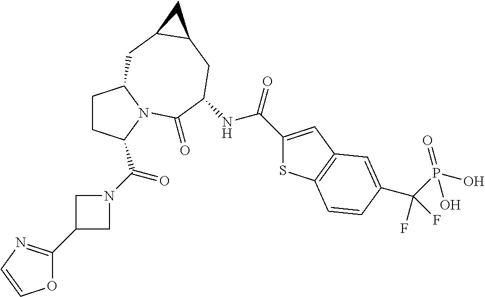 [SMILES: O=C(N[C@H]1C[C@@H]2C[C@@H]2C[C@H]2CC[C@@H](C(=O)N3CC(c4ncco4)C3)N2C1=O)c1cc2cc(C(F)(F)P(=O)(O)O)ccc2s1] | 635.1 | ¹H NMR (400 MHz, CD₃OD) δ 8.16-8.09 (m, 2H), 7.99 (m, 1H), 7.87 (d, J = 19.6 Hz, 1H), 7.69-7.63 (m, 1H), 7.24-7.00 (m, 1H), 5.08-5.00 (m, 1H), 4.56-4.39 (m, 2H), 4.33-4.24 (m, 2H), 4.21-3.91 (m, 2H), 3.68-3.51 (m, 1H), 2.51-2.38 (m, 1H), 2.34-2.13 (m, 4H), 2.02-1.90 (m, 2H), 1.78-1.66 (m, 1H), 1.42 (m, 1H), 1.00-0.89 (m, 1H), 0.88-0.80 (m, |
| | | | | 1H), 0.09-0.03 (m, |
| | | | | 1H) |
| 299 | (difluoro(2- (((3S,6S,7aS, 8aR,9aR)-3- (3-(isoxazol- 3- yl)azetidine- 1-carbonyl)- 5-oxodeca- hydro-1H- cyclopropa[d] pyrrolo[1,2- a]azocin-6- yl)carbamoyl) benzo[b] thiophen-5- yl)methyl) | 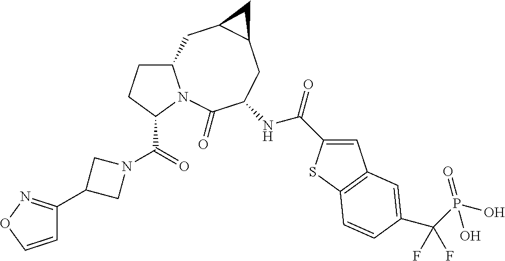 [SMILES: O=C(N[C@H]1C[C@@H]2C[C@@H]2C[C@H]2CC[C@@H](C(=O)N3CC(c4ccon4)C3)N2C1=O)c1cc2cc(C(F)(F)P(=O)(O)O)ccc2s1] | 635.1 | ¹H NMR (400 MHz, CD₃OD) δ 8.66-8.56 (m, 1H), 8.13 (d, J = 11.2 Hz, 2H), 7.99 (t, J = 8.4 Hz, 1H), 7.66 (dd, J = 3.6, 8.0 Hz, 1H), 6.70-6.53 (m, 1H), 5.08-4.91 (m, 2H), 4.72-4.64 (m, 1H), 4.56-4.49 (m, 1H), 4.46-4.34 (m, 1H), 4.33-4.25 (m, 1H), 4.16-3.98 (m, 2H), 2.52-2.40 (m, 1H), 2.35-2.08 (m, 4H), 2.03-1.90 (m, |
| | phosphonic | | | 2H), 1.80-1.67 (m, |
| | acid | | | 1H), 1.49-1.35 (m, |
| | | | | 1H), 1.01-0.90 (m, |
| | | | | 1H), 0.84 (td, J = 3.6, |
| | | | | 7.2 Hz, 1H), 0.06 (d, |
| | | | | J = 4.8 Hz, 1H) |
| 294 | ((2- (((3S,6S,7aR, 8aS,9aR)-3- (3-(4- hydroxy- pyridin-3- yl)azetidine- 1-carbonyl)- 5-oxodeca- hydro-1H- cyclopropa[d] pyrrolo[1,2- a]azocin-6- yl)carbamoyl) benzo[b] thiophen-5- yl)methyl) phosphonic acid | 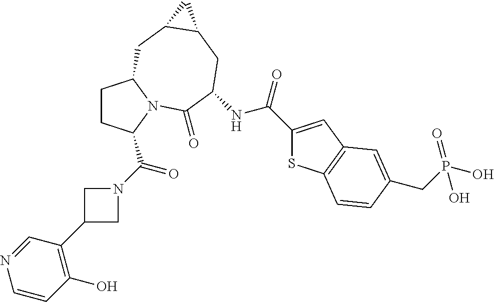 [SMILES: O=C(N[C@H]1C[C@H]2C[C@H]2C[C@H]2CC[C@@H](C(=O)N3CC(c4cnccc4O)C3)N2C1=O)c1cc2cc(CP(=O)(O)O)ccc2s1] | 625.3 | ¹H NMR (400 MHz, CD₃OD) δ 8.30-7.99 (m, 2H), 7.96-7.79 (m, 2H), 7.72-7.67 (m, 1H), 7.46-7.35 (m, 1H), 7.02-6.60 (m, 1H), 4.98-4.92 (m, 1H), 4.74-4.67 (m, 1H), 4.56 (t, J = 8.0 Hz, 1H), 4.47- 4.28 (m, 3H), 4.18- 4.10 (m, 1H), 4.09- 4.00 (m, 1H), 3.28- 3.17 (m, 2H), 2.42- 2.33 (m, 1H), 2.31- 2.11 (m, 3H), 2.05- 1.81 (m, 3H), 1.73- 1.54 (m, 1H), 1.50- 1.39 (m, 1H), 1.34- |
| | | | | 1.28 (m, 1H), 0.93- |
| | | | | 0.83 (m, 1H), 0.23- |
| | | | | 0.16 (m, 1H) |
| 293 | (fluoro(2- (((3S,6S,7aR, 8aS,9aR)-3- (3-(4- hydroxy- pyridin-3- yl)azetidine- 1-carbonyl)- 5-oxodeca- hydro-1H- cyclopropa[d] pyrrolo[1,2- a]azocin-6- yl)carbamoyl) benzo[b] thiophen-5- yl)methyl) phosphonic acid | 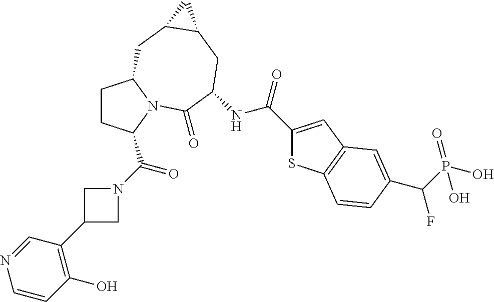 [SMILES: O=C(N[C@H]1C[C@H]2C[C@H]2C[C@H]2CC[C@@H](C(=O)N3CC(c4cnccc4O)C3)N2C1=O)c1cc2cc(C(F)P(=O)(O)O)ccc2s1] | 643.3 | ¹H NMR (400 MHz, CD₃OD) δ 8.35-8.03 (m, 2H), 7.99-7.73 (m, 2H), 7.69-7.38 (m, 3H), 5.88-5.59 (m, 1H), 4.76 (t, J = 9.2 Hz, 2H), 4.61- 4.54 (m, 1H), 4.46- 4.40 (m, 1H), 4.39- 4.29 (m, 2H), 4.24- 4.12 (m, 1H), 4.06- 3.95 (m, 1H), 2.47- 2.35 (m, 1H), 2.27- 2.18 (m, 2H), 2.07- 1.88 (m, 3H), 1.52- 1.36 (m, 2H), 1.36- 1.26 (m, 2H), 0.95- 0.87 (m, 1H), 0.26- 0.07 (m, 1H) |
| 288 | ((2- (((3S,6S,7aS, 8aR,9aR)-3- (3-(1H- pyrazol-4- yl)azetidine- 1-carbonyl)- 5-oxodeca- hydro-1H- cyclopropa[d] pyrrolo[1,2- a]azocin-6- yl)carbamoyl) benzo[b] thiophen-5- yl)difluoro- | 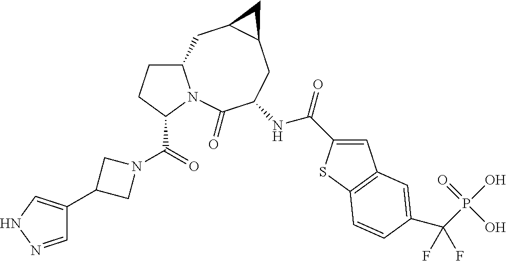 [SMILES: O=C(N[C@H]1C[C@@H]2C[C@@H]2C[C@H]2CC[C@@H](C(=O)N3CC(c4cn[nH]c4)C3)N2C1=O)c1cc2cc(C(F)(F)P(=O)(O)O)ccc2s1] | 634.2 | ¹H NMR (400 MHz, DMSO-d₆) δ 9.00- 8.86 (m, 1H), 8.32 (d, J = 3.6 Hz, 1H), 8.14- 8.02 (m, 2H), 7.860- 7.846 (m, J = 1.8, 7.2 Hz, 1H), 7.63- 7.52 (m, 2H), 6.28 (d, J = 5.9 Hz, 1H), 5.39- 5.13 (m, 1H), 4.98- 4.91 (m, 1H), 4.80- 4.55 (m, 1H), 4.48- 4.29 (m, 2H), 4.26- 4.12 (m, 2H), 4.175- 4.086 (m, J = 5.0, 9.6 Hz, 1H), 2.39-2.28 |
| | methyl) | | | (m, 1H), 2.22-1.99 |
| | phosphonic | | | (m, 4H), 1.94-1.77 |
| | acid | | | (m, 2H), 1.73-1.56 |
| | | | | (m, 1H), 1.43-1.30 |
| | | | | (m, 1H), 0.87-0.66 |
| | | | | (m, 2H), 0.08-−0.05 |
| | | | | (m, 1H) |
| 286 | (difluoro(2- (((3S,6S,7aS, 8aR,9aR)-5- oxo-3-(3- (thiazol-2- yl)azetidine- 1- carbonyl) decahydro-1H- cyclopropa[d] pyrrolo[1,2- a]azocin-6- yl)carbamoyl) benzo[b] thiophen-5- yl)methyl) | 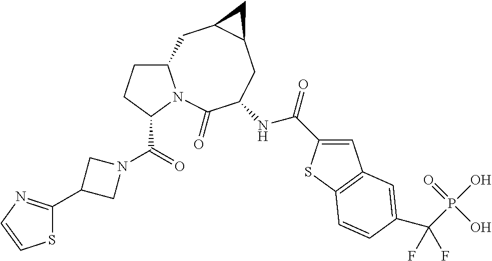 [SMILES: O=C(N[C@H]1C[C@@H]2C[C@@H]2C[C@H]2CC[C@@H](C(=O)N3CC(c4nccs4)C3)N2C1=O)c1cc2cc(C(F)(F)P(=O)(O)O)ccc2s1] | 651.1 | ¹H NMR (400 MHz, CD₃OD) δ 8.08-8.17 (m, 2H), 7.98 (dd, J = 13.60, 8.40 Hz, 1H), 7.64-7.81 (m, 2H), 7.50-7.58 (m, 1H), 4.99-5.08 (m, 2H), 4.79 (s, 1H), 4.52-4.58 (m, 1H), 4.42-4.50 (m, 1H), 4.24-4.36 (m, 2H), 4.07-4.19 (m, 1H), 2.40-2.53 (m, 1H), 2.15-2.33 (m, 4H), 1.89-2.08 (m, 2H), 1.74 (m, 1H), 1.23- |
| | phosphonic | | | 1.48 (m, 1H), 0.77- |
| | acid | | | 1.03 (m, 2H), −0.02- |
| | | | | 0.15 (m, 1H) |
| 282 | (difluoro(2- (((3S,6S,7aS, 8aR,9aR)-3- (3-(1-methyl- 1H-pyrazol- 4- yl)azetidine- 1-carbonyl)- 5-oxodeca- hydro-1H- cyclopropa[d] pyrrolo[1,2- a]azocin-6- yl)carbamoyl) | 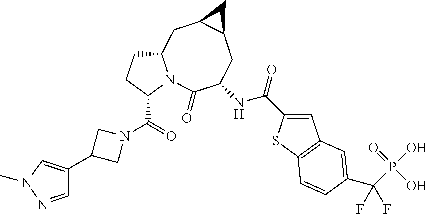 [SMILES: Cn1cc(C2CN(C(=O)[C@@H]3CC[C@@H]4C[C@H]5C[C@H]5C[C@H](NC(=O)c5cc6cc(C(F)(F)P(=O)(O)O)ccc6s5)C(=O)N43)C2)cn1] | 648 | ¹H NMR (400 MHz, CD₃OD) δ 8.18-8.13 (m, 2H), 8.01 (d, J = 8.4 Hz, 1H), 7.70- 7.64 (m, 2H), 7.54 (d, J = 16.4 Hz, 1H), 5.06- 5.02 (m, 1H), 4.91 (s, 1H), 4.61-4.49 (m, 2H), 4.39-4.26 (m, 2H), 3.90-3.77 (m, 5H), 2.51-2.41 (m, 1H), 2.34-2.11 (m, 4H), 2.02-1.93 (m, 2H), 1.79-1.69 |
| | benzo[b] | | | (m, 1H), 1.45-1.42 |
| | thiophen-5- | | | (m, 1H), 1.02-0.91 |
| | yl)methyl) | | | (m, 1H), 0.87-0.82 |
| | phosphonic | | | (m, 1H), 0.09-0.05 |
| | acid | | | (m, 1H) |
| 280 | (fluoro(2- (((3S,6S,7aS, 8aR,9aR)-3- (3-(4- methyl- pyridin-3- yl)azetidine- 1-carbonyl)- 5-oxodeca- hydro-1H- cyclopropa[d] pyrrolo[1,2- a]azocin-6- yl)carbamoyl) benzo[b] thiophen-5- yl)methyl) phosphonic acid | 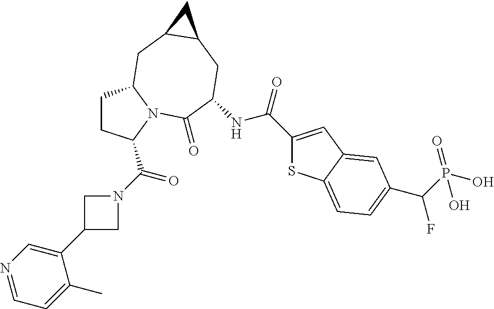 [SMILES: Cc1ccncc1C1CN(C(=O)[C@@H]2CC[C@@H]3C[C@H]4C[C@H]4C[C@H](NC(=O)c4cc5cc(C(F)P(=O)(O)O)ccc5s4)C(=O)N32)C1] | 641.1 | ¹H NMR (400 MHz, DMSO-d₆) δ 8.91- 8.79 (m, 1H), 8.59- 8.49 (m, 1H), 8.35- 8.20 (m, 2H), 8.03- 7.88 (m, 2H), 7.52- 7.44 (m, 1H), 7.20- 7.17 (m, 1H), 5.72- 5.52 (m, 1H), 4.96- 4.90 (m, 1H), 4.62 (d, J = 8.0 Hz, 1H), 4.42- 4.39 (m, 1H), 4.35- 4.27 (m, 1H), 4.24- 4.13 (m, 2H), 4.11- 3.96 (m, 2H), 2.35- 2.29 (m, 1H), 2.19 (s, 3H), 2.17-2.05 (m, 3H), 2.05-1.96 (m, 1H), 1.91-1.78 (m, |
| | | | | 2H), 1.69-1.58 (m, |
| | | | | 1H), 1.37 (d, J = 2.4 |
| | | | | Hz, 1H), 0.86-0.67 |
| | | | | (m, 2H), 0.03-−0.04 |
| | | | | (m, 1H) |
| 277 | ((2- (((3S,6S,7aS, 8aR,9aR)-3- (3-(4- methyl- pyridin-3- yl)azetidine- 1-carbonyl)- 5-oxodeca- hydro-1H- cyclopropa[d] pyrrolo[1,2- a]azocin-6- yl)carbamoyl) benzo[b] thiophen-5- yl)methyl) phosphonic acid | 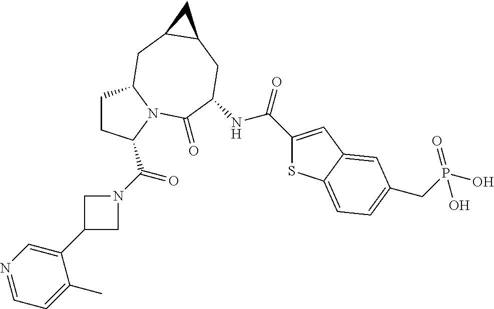 [SMILES: Cc1ccncc1C1CN(C(=O)[C@@H]2CC[C@@H]3C[C@H]4C[C@H]4C[C@H](NC(=O)c4cc5cc(CP(=O)(O)O)ccc5s4)C(=O)N32)C1] | 623.2 | ¹H NMR (400 MHz, DMSO-d₆) δ 8.88- 8.74 (m, 1H), 8.58- 8.48 (m, 1H), 8.36- 8.29 (m, 1H), 8.18 (d, J = 16.6 Hz, 1H), 7.89- 7.82 (m, 1H), 7.80- 7.68 (m, 1H), 7.37- 7.29 (m, 1H), 7.21- 7.15 (m, 1H), 4.96- 4.87 (m, 1H), 4.62 (d, J = 7.6 Hz, 1H), 4.43- 4.37 (m, 1H), 4.35- 4.26 (m, 1H), 4.24- 4.13 (m, 2H), 4.07 (d, J = 7.6 Hz, 1H), 4.02- 3.96 (m, 1H), 3.02- 2.96 (m, 2H), 2.36- 2.29 (m, 1H), 2.19 (s, |
| | | | | 3H), 2.11 (d, J = 5.2 |
| | | | | Hz, 2H), 2.00 (d, J = |
| | | | | 8.0 Hz, 1H), 1.91- |
| | | | | 1.77 (m, 2H), 1.69- |
| | | | | 1.57 (m, 1H), 1.41- |
| | | | | 1.32 (m, 1H), 1.23 (d, |
| | | | | J = 6.4 Hz, 1H), 0.86- |
| | | | | 0.66 (m, 2H), 0.07- |
| | | | | −0.05 (m, 1H) |
| 272 | (fluoro(2- (((3S,6S,7aS, 8aR,9aR)-3- (3-(4- methoxy- pyridin-3- yl)azetidine- 1-carbonyl)- 5-oxodeca- hydro-1H- cyclopropa[d] pyrrolo[1,2- a]azocin-6- yl)carbamoyl) | 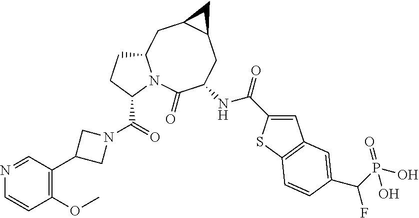 [SMILES: COc1ccncc1C1CN(C(=O)[C@@H]2CC[C@@H]3C[C@H]4C[C@H]4C[C@H](NC(=O)c4cc5cc(C(F)P(=O)(O)O)ccc5s4)C(=O)N32)C1] | 657 | ¹H NMR (400 MHz, DMSO-d₆) δ 8.85- 8.82 (m, 1H), 8.57- 8.44 (m, 2H), 8.27 (d, J = 18.8 Hz, 1H), 8.06- 7.94 (m, 2H), 7.59- 7.49 (m, 1H), 7.32- 7.20 (m, 1H), 5.90- 5.73 (m, 1H), 4.98- 4.89 (m, 1H), 4.55 (d, J = 7.2 Hz, 1H), 4.39- 4.26 (m, 2H), 4.22- 4.11 (m, 2H), 4.05- 3.92 (m, 5H), 2.37- |
| | benzo[b] | | | 2.29 (m, 1H), 2.17- |
| | thiophen-5- | | | 1.97 (m, 4H), 1.92- |
| | yl)methyl) | | | 1.79 (m, 2H), 1.71- |
| | phosphonic | | | 1.56 (m, 1H), 1.44- |
| | acid | | | 1.29 (m, 1H), 0.86- |
| | | | | 0.67 (m, 2H), 0.04- |
| | | | | −0.03 (m, 1H) |
| 267 | (fluoro(2- (((3S,6S,7aR, 8aS,9aR)-3- (((3S,4S)-4- fluoro-1- (pyridin-3- yl)pyrrolidin- 3-yl) carbamoyl)-5- oxodecahydro- 1H- cyclopropa[d] pyrrolo[1,2- a]azocin-6- yl)carbamoyl) benzo[b] thiophen-5- yl)methyl) | 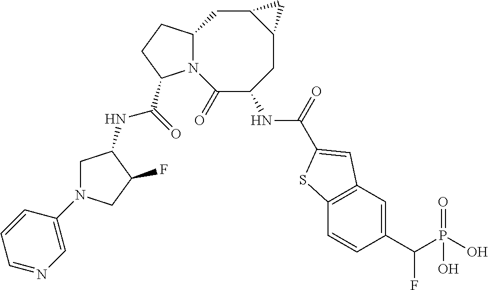 [SMILES: O=C(N[C@H]1C[C@H]2C[C@H]2C[C@H]2CC[C@@H](C(=O)N[C@H]3CN(c4cccnc4)C[C@@H]3F)N2C1=O)c1cc2cc(C(F)P(=O)(O)O)ccc2s1] | 674.2 | ¹H NMR (400 MHz, DMSO-d₆) δ 8.76 (d, J = 7.6 Hz, 1H), 8.45 (d, J = 6.8 Hz, 1H), 8.22 (s, 1H), 8.01- 7.96 (m, 2H), 7.96- 7.86 (m, 2H), 7.49 (d, J = 8.4 Hz, 1H), 7.20- 7.17 (m, 1H), 6.98 (d, J = 8.0 Hz, 1H), 5.75-5.60 (m, 1H), 5.19-5.03 (m, 1H), 4.81 (d, J = 6.8 Hz, 1H), 4.45-4.37 (m, 1H), 4.27-4.13 (m, 3H), 3.67 (d, J = 3.2 Hz, 2H), 3.26 (d, J = 10.4 Hz, 2H), 2.13- |
| | phosphonic | | | 1.98 (m, 4H), 1.93- |
| | acid | | | 1.81 (m, 2H), 1.72- |
| | | | | 1.55 (m, 2H), 1.39- |
| | | | | 1.29 (m, 1H), 1.26- |
| | | | | 1.16 (m, 1H), 0.81- |
| | | | | 0.74 (m, 1H), 0.11- |
| | | | | 0.03 (m, 1H) |
| 266 | ((2- (((3S,6S,7aS, 8aR,9aR)-3- (3-(4- ethylpyridin- 3- yl)azetidine- 1-carbonyl)- 5-oxodeca- hydro-1H- cyclopropa[d] pyrrolo[1,2- a]azocin-6- yl)carbamoyl) benzo[b] thiophen-5- yl)fluoro- methyl) phosphonic | 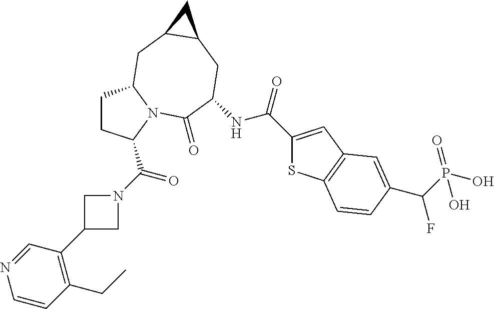 [SMILES: CCc1ccncc1C1CN(C(=O)[C@@H]2CC[C@@H]3C[C@H]4C[C@H]4C[C@H](NC(=O)c4cc5cc(C(F)P(=O)(O)O)ccc5s4)C(=O)N32)C1] | 655 | ¹H NMR (400 MHz, CD₃OD) δ 8.87- 8.63 (m, 1H), 8.53- 8.29 (m, 1H), 8.03- 7.89 (m, 2H), 7.88- 7.66 (m, 2H), 7.64- 7.52 (m, 1H), 5.87- 5.69 (m, 1H), 5.12- 4.96 (m, 2H), 4.80- 4.70 (m, 1H), 4.63- 4.50 (m, 1H), 4.46- 4.26 (m, 3H), 4.11 (s, 1H), 2.86-2.70 (m, 2H), 2.55-2.42 (m, 1H), 2.36-2.10 (m, 4H), 2.05-1.92 (m, 2H), 1.81-1.69 (m, 1H), 1.48-1.36 (m, 1H), 1.31-1.19 (m, |
| | acid | | | 3H), 1.01-0.79 (m, |
| | | | | 2H), 0.12-0.04 (m, |
| | | | | 1H) |
| 265 | ((2- (((3S,6S,7aS, 8aR,9aR)-3- (3-(4- methoxy- pyridin-3- yl)azetidine- 1-carbonyl)- 5-oxodeca- hydro-1H- cyclopropa[d] pyrrolo[1,2- a]azocin-6- yl)carbamoyl) benzo[b] thiophen-5- yl)methyl) phosphonic acid | 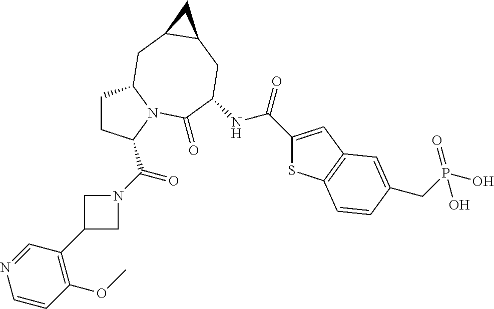 [SMILES: COc1ccncc1C1CN(C(=O)[C@@H]2CC[C@@H]3C[C@H]4C[C@H]4C[C@H](NC(=O)c4cc5cc(CP(=O)(O)O)ccc5s4)C(=O)N32)C1] | 639.1 | ¹H NMR (400 MHz, DMSO-d₆) δ 8.80- 8.59 (m, 3H), 8.24- 8.16 (m, 1H), 7.98- 7.87 (m, 1H), 7.86- 7.76 (m, 1H), 7.55- 7.49 (m, 1H), 7.43- 7.33 (m, 1H), 4.98- 4.79 (m, 2H), 4.56 (d, J = 8.4 Hz, 1H), 4.39- 4.29 (m, 2H), 4.22- 4.14 (m, 2H), 4.08- 4.03 (m, 4H), 3.13- 3.06 (m, 2H), 2.36- 2.27 (m, 1H), 2.15 (d, J = 3.2 Hz, 1H), 2.08 (s, 2H), 1.91-1.79 (m, 2H), 1.68-1.59 (m, 1H), 1.41-1.31 |
| | | | | (m, 1H), 1.35-1.23 |
| | | | | (m, 1H), 0.85-0.70 |
| | | | | (m, 2H), 0.03-−0.03 |
| | | | | (m, 1H) |
| 262 | (fluoro(2- (((3S,6S,7aS, 8aR,9aR)-3- (3-(4- fluoro- pyridin-3- yl)azetidine- 1-carbonyl)- 5-oxodeca- hydro-1H- cyclopropa[d] pyrrolo[1,2- a]azocin-6- yl)carbamoyl) benzo[b] thiophen-5- yl)methyl) phosphonic acid | 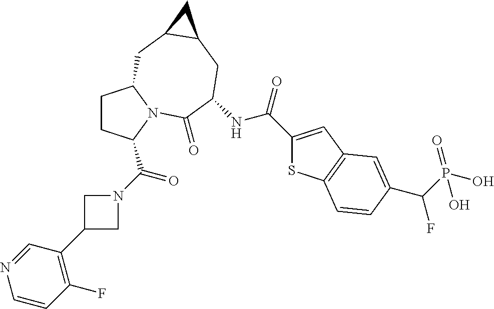 [SMILES: O=C(N[C@H]1C[C@@H]2C[C@@H]2C[C@H]2CC[C@@H](C(=O)N3CC(c4cnccc4F)C3)N2C1=O)c1cc2cc(C(F)P(=O)(O)O)ccc2s1] | 645.1 | ¹H NMR (400 MHz, DMSO-d6) δ 9.03- 8.76 (m, 1H), 8.73- 8.61 (m, 1H), 8.58- 8.46 (m, 1H), 8.37- 8.21 (m, 1H), 8.12- 7.92 (m, 2H), 7.64- 7.45 (m, 1H), 7.32 (dt, J = 5.6, 10.4 Hz, 1H), 5.94-5.64 (m, 1H), 5.02-4.82 (m, 1H), 4.62 (d, J = 8.0 Hz, 1H), 4.44-3.98 (m, 5H), 2.37-2.28 (m, 1H), 2.21-1.94 (m, 4H), 1.92-1.78 (m, 2H), 1.73-1.57 (m, 1H), 1.44-1.29 (m, 1H), 1.28-1.17 |
| | | | | (m, 1H), 0.90-0.61 |
| | | | | (m, 2H), −0.01 (d, J = |
| | | | | 4.0 Hz, 1H) |
| 254 | ((2- (((3S,6S,7aR, 8aS,9aR)-3- (((3S,4S)-1- acetyl-4- fluoro- pyrrolidin-3- yl) carbamoyl)-5- oxodecahydro- 1H- cyclopropa[d] pyrrolo[1,2- a]azocin-6- yl)carbamoyl) benzo[b] thiophen-5- yl)fluoro- | 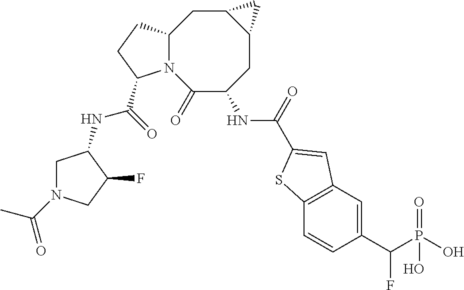 [SMILES: CC(=O)N1C[C@H](NC(=O)[C@@H]2CC[C@@H]3C[C@@H]4C[C@@H]4C[C@H](NC(=O)c4cc5cc(C(F)P(=O)(O)O)ccc5s4)C(=O)N32)[C@@H](F)C1] | 639.1 | ¹H NMR (400 MHz, CD₃OD) δ 8.04 (d, J = 17.6 Hz, 2H), 7.94 (d, J = 8.4 Hz, 1H), 7.63- 7.56 (m, 1H), 5.94- 5.67 (m, 1H), 5.18- 4.97 (m, 1H), 4.97- 4.93 (m, 1H), 4.53- 4.36 (m, 1H), 4.36- 4.28 (m, 2H), 3.93- 3.82 (m, 1H), 3.78- 3.58 (m, 3H), 2.42- 2.30 (m, 1H), 2.26- 2.11 (m, 3H), 2.08 (d, J = 6.0 Hz, 3H), 2.03- 1.90 (m, 2H), 1.89- 1.80 (m, 1H), 1.80- 1.69 (m, 1H), 1.53- |
| | methyl) | | | 1.40 (m, 1H), 1.34- |
| | phosphonic | | | 1.23 (m, 1H), 0.91- |
| | acid | | | 0.81 (m, 1H), 0.26- |
| | | | | 0.15 (m, 1H) |
| 253 | ((2- (((3S,6S,7aR, 8aS,9aR)-3- (((3S,4S)-4- fluoro-1- (pyridin-3- yl)pyrrolidin- 3-yl) carbamoyl)-5- oxodecahydro- 1H- cyclopropa[d] pyrrolo[1,2- a]azocin-6- yl)carbamoyl) benzo[b] thiophen-5- | 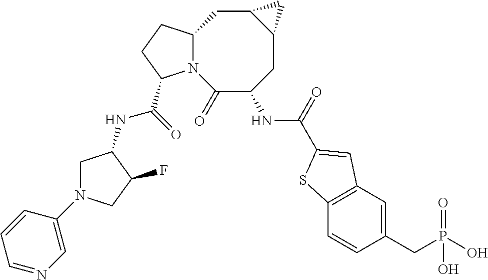 [SMILES: O=C(N[C@H]1C[C@H]2C[C@H]2C[C@H]2CC[C@@H](C(=O)N[C@H]3CN(c4cccnc4)C[C@@H]3F)N2C1=O)c1cc2cc(CP(=O)(O)O)ccc2s1] | 656.1 | ¹H NMR (400 MHz, DMSO-d₆) δ 8.69 (d, J = 7.2 Hz, 1H), 8.44 (d, J = 6.8 Hz, 1H), 8.16-8.13 (m, 2H), 8.08 (d, J = 5.2 Hz, 1H), 7.90 (d, J = 8.2 Hz, 1H), 7.79 (s, 1H), 7.68 (d, J = 5.2 Hz, 1H), 7.59 (s, 1H), 7.36 (d, J = 8.2 Hz, 1H), 5.27-5.10 (m, 1H), 4.79 (t, J = 5.6 Hz, 1H), 4.49-4.40 (m, 1H), 4.19 (d, J = 8.0 Hz, 2H), 3.78- 3.61 (m, 4H), 3.37 (d, |
| | yl)methyl) | | | J = 10.8 Hz, 1H), 3.14- |
| | phosphonic | | | 3.05 (m, 2H), 2.28- |
| | acid | | | 2.24 (m, 1H), 2.14- |
| | | | | 1.97 (m, 4H), 1.93- |
| | | | | 1.81 (m, 2H), 1.71- |
| | | | | 1.66 (m, 1H), 1.64- |
| | | | | 1.54 (m, 1H), 1.38- |
| | | | | 1.15 (m, 3H), 0.80- |
| | | | | 0.75 (m, 1H), 0.04- |
| | | | | 0.03 (m, 1H) |
| 252 | ((2- (((3S,6S,7aS, 8aR,9aR)-3- (3-(4- ethylpyridin- 3- yl)azetidine- 1-carbonyl)- 5-oxodeca- hydro-1H- cyclopropa[d] pyrrolo[1,2- a]azocin-6- | 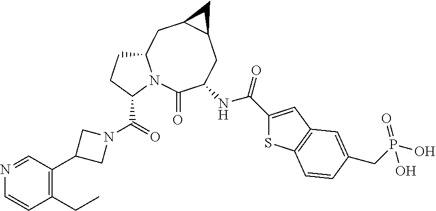 [SMILES: CCc1ccncc1C1CN(C(=O)[C@@H]2CC[C@@H]3C[C@H]4C[C@H]4C[C@H](NC(=O)c4cc5cc(CP(=O)(O)O)ccc5s4)C(=O)N32)C1] | 637.1 | ¹H NMR (400 MHz, CD₃OD) δ 8.86- 8.74 (m, 1H), 8.54- 8.48 (m, 1H), 7.99 (d, J = 3.6 Hz, 1H), 7.79 (d, J = 8.4 Hz, 3H), 7.45-7.38 (m, 1H), 5.10-4.97 (m, 1H), 4.86-4.68 (m, 2H), 4.57-4.51 (m, 1H), 4.46-4.26 (m, 3H), 4.14-4.08 (m, 1H), 3.27-3.17 (m, 2H), |
| | yl)carbamoyl) | | | 2.85-2.75 (m, 2H), |
| | benzo[b] | | | 2.52-2.40 (m, 1H), |
| | thiophen-5- | | | 2.35-2.11 (m, 4H), |
| | yl)methyl) | | | 2.06-1.93 (m, 2H), |
| | phosphonic | | | 1.80-1.70 (m, 1H), |
| | acid | | | 1.48-1.36 (m, 1H), |
| | | | | 1.33-1.23 (m, 3H), |
| | | | | 1.00-0.81 (m, 2H), |
| | | | | 0.12-0.05 (m, 1H) |
| 250 | ((2- (((3S,6S,7aR, 8aS,9aR)-3- (3-(4- fluoro- pyridin-3- yl)azetidine- 1-carbonyl)- 5-oxodeca- hydro-1H- cyclopropa[d] pyrrolo[1,2- a]azocin-6- | 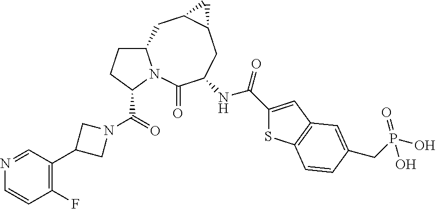 [SMILES: O=C(N[C@H]1C[C@H]2C[C@H]2C[C@H]2CC[C@@H](C(=O)N3CC(c4cnccc4F)C3)N2C1=O)c1cc2cc(CP(=O)(O)O)ccc2s1] | 627 | ¹H NMR (400 MHz, CD₃OD) δ 8.89-8.70 (m, 1H), 8.60 (q, J = 6.4 Hz, 1H), 8.01 (d, J = 11.2 Hz, 1H), 7.88- 7.79 (m, 2H), 7.51- 7.38 (m, 2H), 4.97- 4.89 (m, 2H), 4.75- 4.64 (m, 1H), 4.52- 4.44 (m, 1H), 4.44- 4.39 (m, 1H), 4.36- 4.29 (m, 1H), 4.28- 4.11 (m, 2H), 3.29- |
| | yl)carbamoyl) | | | 3.25 (m, 1H), 3.24- |
| | benzo[b] | | | 3.19 (m, 1H), 2.40- |
| | thiophen-5- | | | 2.31 (m, 1H), 2.30- |
| | yl)methyl) | | | 2.23 (m, 1H), 2.22- |
| | phosphonic | | | 2.11 (m, 2H), 2.05- |
| | acid | | | 1.93 (m, 2H), 1.92- |
| | | | | 1.80 (m, 1H), 1.77- |
| | | | | 1.64 (m, 1H), 1.51- |
| | | | | 1.40 (m, 1H), 1.34- |
| | | | | 1.23 (m, 1H), 0.86 |
| | | | | (dt, J = 4.8, 8.0 Hz, |
| | | | | 1H), 0.18 (q, J = 4.8 |
| | | | | Hz, 1H) |
| 248 | (fluoro(2- (((3S,6S,7aR, 8aS,9aR)-3- (3-(4- fluoropyridin- 3- yl)azetidine- 1-carbonyl)- 5-oxodeca- hydro-1H- cyclopropa[d] pyrrolo[1,2- a]azocin-6- | 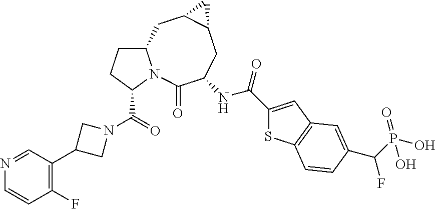 [SMILES: O=C(N[C@H]1C[C@H]2C[C@H]2C[C@H]2CC[C@@H](C(=O)N3CC(c4cnccc4F)C3)N2C1=O)c1cc2cc(C(F)P(=O)(O)O)ccc2s1] | 645 | ¹H NMR (400 MHz, DMSO-d₆) δ 8.84 (dd, J = 7.6, 13.2 Hz, 1H), 8.71 (d, J = 10.0 Hz, 1H), 8.62-8.51 (m, 1H), 8.26 (d, J = 12.8 Hz, 1H), 8.08- 8.02 (m, 1H), 8.00- 7.97 (m, 1H), 7.58- 7.51 (m, 1H), 7.39 (ddd, J = 6.0, 10.4, 18.4 Hz, 1H), 5.91- 5.77 (m, 1H), 4.82 (s, |
| | yl)carbamoyl) | | | 1H), 4.65-4.47 (m, |
| | benzo[b] | | | 1H), 4.40-4.28 (m, |
| | thiophen-5- | | | 2H), 4.27-4.15 (m, |
| | yl)methyl) | | | 2H), 4.14-3.98 (m, |
| | phosphonic | | | 2H), 2.29-2.16 (m, |
| | acid | | | 2H), 2.11-1.97 (m, |
| | | | | 2H), 1.95-1.82 (m, |
| | | | | 2H), 1.74-1.61 (m, |
| | | | | 1H), 1.59-1.46 (m, |
| | | | | 1H), 1.38-1.27 (m, |
| | | | | 1H), 1.26-1.14 (m, |
| | | | | 1H), 0.80-0.72 (m, |
| | | | | 1H), 0.05 (d, J = 4.8 |
| | | | | Hz, 1H) |
| 247 | ((2- (((3S,6S,7aS, 8aR,9aR)-3- (3-(4- fluoropyridin- 3- yl)azetidine- 1-carbonyl)- 5-oxodeca- hydro-1H- cyclopropa[d] pyrrolo[1,2- a]azocin-6- yl)carbamoyl) | 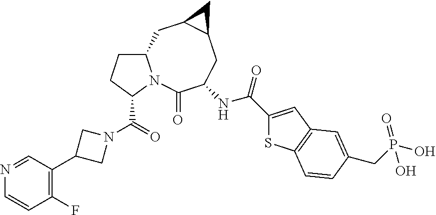 [SMILES: O=C(N[C@H]1C[C@@H]2C[C@@H]2C[C@H]2CC[C@@H](C(=O)N3CC(c4cnccc4F)C3)N2C1=O)c1cc2cc(CP(=O)(O)O)ccc2s1] | 627.1 | ¹H NMR (400 MHz, CD₃OD) δ 8.70-8.93 (m, 1H) 8.63 (m, 1H) 8.04 (d, J = 12.0 Hz, 1H) 7.78-7.87 (m, 2H) 7.37-7.54 (m, 2H) 4.99-5.05 (m, 1H) 4.73 (s, 1H) 4.06- 4.64 (m, 6H) 3.21- 3.28 (m, 2H) 2.13 (s, 5H) 1.89-2.06 (m, 2H) 1.66-1.82 (m, 1H) 1.31-1.52 (m, 1H) 0.80-1.02 (m, |
| | benzo[b] | | | 2H) 0.01-0.12 (m, |
| | thiophen-5- | | | 1H) |
| | yl)methyl) | | | |
| | phosphonic | | | |
| | acid | | | |
| 244 | ((2- (((3S,6S,7aR, 8aS,9aR)-3- (((3S,4S)-1- acetyl-4- fluoro- pyrrolidin-3- yl) carbamoyl)-5- oxodecahydro- 1H- cyclopropa[d] pyrrolo[1,2- a]azocin-6- yl)carbamoyl) benzo[b] thiophen-5- yl)methyl) | 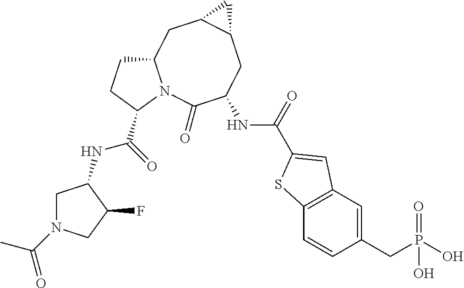 [SMILES: CC(=O)N1C[C@H](NC(=O)[C@@H]2CC[C@@H]3C[C@@H]4C[C@@H]4C[C@H](NC(=O)c4cc5cc(CP(=O)(O)O)ccc5s4)C(=O)N32)[C@@H](F)C1] | 621.2 | ¹H NMR (400 MHz, CD₃OD) δ 8.00 (s, 1H), 7.89-7.78 (m, 2H), 7.43 (d, J = 8.4 Hz, 1H), 5.17-4.96 (m, 1H), 4.94 (t, J = 4.4 Hz, 1H), 4.49- 4.37 (m, 1H), 4.36- 4.28 (m, 2H), 3.92- 3.82 (m, 1H), 3.79- 3.71 (m, 1H), 3.71- 3.58 (m, 2H), 3.28 (s, 1H), 3.23 (s, 1H), 2.42-2.28 (m, 1H), 2.24-2.11 (m, 3H), 2.08 (d, J = 6.4 Hz, 3H), 2.00-1.90 (m, 2H), 1.89-1.80 (m, |
| | phosphonic | | | 1H), 1.79-1.68 (m, |
| | acid | | | 1H), 1.52-1.40 (m, |
| | | | | 1H), 1.36-1.24 (m, |
| | | | | 1H), 0.90-0.81 (m, |
| | | | | 1H), 0.25-0.17 (m, |
| | | | | 1H) |
| 428 | ((S)-(2- (((3S,6S,7aS, 8aR,9aR)-3- (3-(4-(3- cyano- azetidin-1- yl)pyridin-3- yl)azetidine- 1-carbonyl)- 5-oxodeca- hydro-1H- cyclopropa[d] pyrrolo[1,2- a]azocin-6- yl)carbamoyl) benzo[b] | 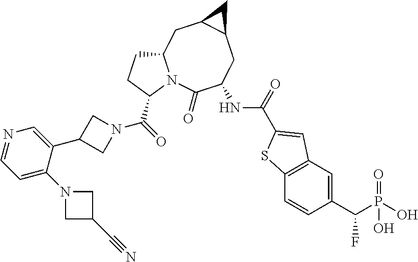 [SMILES: N#CC1CN(c2ccncc2C2CN(C(=O)[C@@H]3CC[C@@H]4C[C@H]5C[C@H]5C[C@H](NC(=O)c5cc6cc([C@@H](F)P(=O)(O)O)ccc6s5)C(=O)N43)C2)C1] | 707.2 | 1H NMR (400 MHz, DMSO-d6) δ 8.40- 8.27 (m, 1H), 8.23- 8.10 (m, 1H), 8.05 (s, 1H), 7.88-7.83 (m, 1H), 7.64 (s, 1H), 7.53 (d, J = 8.4 Hz, 1H), 6.58-6.35 (m, 1H), 5.84-5.61 (m, 1H), 4.96-4.88 (m, 1H), 4.75-4.65 (m, 1H), 4.56 (d, J = 4.0 Hz, 1H), 4.50 (d, J = 3.2 Hz, 2H), 4.46- 4.39 (m, 2H), 4.35- 4.30 (m, 1H), 4.25- |
| | thiophen-5- | | | 4.17 (m, 2H), 4.15 (s, |
| | yl)fluoro- | | | 1H), 3.90 (s, 2H), |
| | methyl) | | | 2.15-2.10 (m, 2H), |
| | phosphonic | | | 1.97-1.95 (m, 1H), |
| | acid | | | 1.86-1.84 (m, 2H), |
| | | | | 1.75-1.57 (m, 2H), |
| | | | | 1.43 (d, J = 7.2 Hz, |
| | | | | 1H), 1.23 (s, 2H), |
| | | | | 0.74-0.73 (m, 1H), |
| | | | | 0.03-0.01 (m, 1H) |
| 427 | ((S)- fluoro(2- (((3S,6S,7aS, 8aR,9aR)-3- (3-(1-methyl- 1H- pyrrolo[3,2- c]pyridin-7- yl)azetidine- 1-carbonyl)- 5-oxodeca- hydro-1H- cyclopropa[d] pyrrolo[1,2- a]azocin-6- | 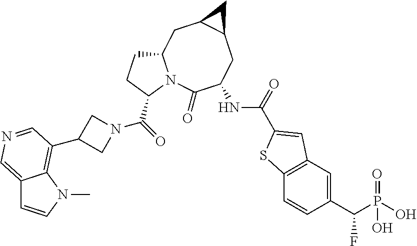 [SMILES: Cn1ccc2cncc(C3CN(C(=O)[C@@H]4CC[C@@H]5C[C@H]6C[C@H]6C[C@H](NC(=O)c6cc7cc([C@@H](F)P(=O)(O)O)ccc7s6)C(=O)N54)C3)c21] | 680.1 | 1H NMR (400 MHz, DMSO-d₆) δ 9.17- 8.72 (m, 1H), 8.40- 8.24 (m, 1H), 8.18- 7.85 (m, 2H), 7.56 (d, J = 2.8 Hz, 4H), 6.74- 6.47 (m, 1H), 5.78- 5.51 (m, 1H), 4.97- 4.85 (m, 2H), 4.82- 4.67 (m, 1H), 4.61 (d, J = 1.2 Hz, 1H), 4.51- 4.41 (m, 1H), 4.29 (t, J = 8.8 Hz, 1H), 4.20-4.06 (m, 2H), 3.93 (s, 3H), 3.78 (s, |
| | yl)carbamoyl) | | | 2H), 2.23-2.11 (m, |
| | benzo[b] | | | 3H), 2.04-1.96 (m, |
| | thiophen-5- | | | 1H), 1.94-1.86 (m, |
| | yl)methyl) | | | 1H), 1.85-1.78 (m, |
| | phosphonic | | | 1H), 1.72-1.59 (m, |
| | acid | | | 1H), 1.51-1.35 (m, |
| | | | | 1H), 1.32-1.23 (m, |
| | | | | 1H), 0.91-0.69 (m, |
| | | | | 2H), 0.04-−0.05 (m, |
| | | | | 1H) |
| 425 | ((S)- fluoro(2- (((3S,6S,7aS, 8aR,9aR)-3- (3-(4-(3- fluoro-3- (methoxy- methyl) azetidin-1-yl) pyridin-3- yl)azetidine- 1-carbonyl)- 5-oxodeca- hydro-1H- cyclopropa[d] pyrrolo[1,2- | 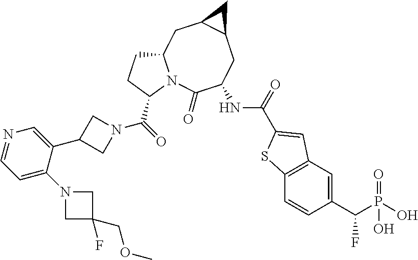 [SMILES: COCC1(F)CN(c2ccncc2C2CN(C(=O)[C@@H]3CC[C@@H]4C[C@H]5C[C@H]5C[C@H](NC(=O)c5cc6cc([C@@H](F)P(=O)(O)O)ccc6s5)C(=O)N43)C2)C1] | 744.4 | 1H NMR (400 MHz, CD₃OD) δ 8.33-7.96 (m, 1H), 7.95-7.88 (m, 1H), 7.81-7.67 (m, 3H), 7.63-7.31 (m, 1H), 6.13 (d, J = 7.2 Hz, 1H), 5.78- 5.61 (m, 1H), 5.00 (t, J = 9.2 Hz, 1H), 4.72- 4.59 (m, 2H), 4.54- 4.42 (m, 3H), 4.33- 4.31 (m, 1H), 4.29- 4.27 (m, 1H), 4.18- 3.94 (m, 3H), 3.79- 3.69 (m, 2H), 3.45 (s, 1H), 3.28 (s, 3H), |
| | a]azocin-6- | | | 2.58-2.44 (m, 1H), |
| | yl)carbamoyl) | | | 2.41-2.34 (m, 1H), |
| | benzo[b] | | | 2.33-2.26 (m, 1H), |
| | thiophen-5- | | | 2.23-2.18 (m, 1H), |
| | yl)methyl) | | | 2.10-2.01 (m, 1H), |
| | phosphonic | | | 2.00-1.88 (m, 2H), |
| | acid | | | 1.81-1.71 (m, 1H), |
| | | | | 1.56-1.38 (m, 1H), |
| | | | | 1.00-0.85 (m, 2H), |
| | | | | 0.12-0.06 (m, 1H) |
| 422 | ((S)- fluoro(2- (((3S,6S,7aS, 8aR,9aR)-3- (3-(4-((R)-3- methoxy- pyrrolidin-1- yl)pyridin-3- yl)azetidine- 1-carbonyl)- 5-oxodeca- hydro-1H- cyclopropa[d] pyrrolo[1,2- a]azocin-6- | 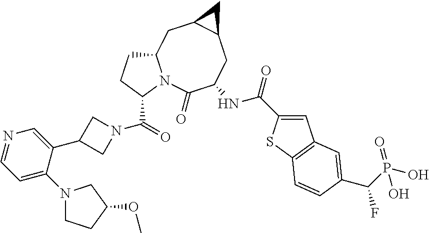 [SMILES: CO[C@@H]1CCN(c2ccncc2C2CN(C(=O)[C@@H]3CC[C@@H]4C[C@H]5C[C@H]5C[C@H](NC(=O)c5cc6cc([C@@H](F)P(=O)(O)O)ccc6s5)C(=O)N43)C2)C1] | 726.2 | 1H NMR (400 MHz, CD₃OD) δ 8.22-7.93 (m, 1H), 7.93-7.85 (m, 2H), 7.81-7.74 (m, 1H), 7.69-7.59 (m, 1H), 7.54-7.37 (m, 1H), 6.75-6.40 (m, 1H), 5.68-5.53 (m, 1H), 4.98-4.83 (m, 2H), 4.66-4.52 (m, 2H), 4.44-4.41 (m, 1H), 4.31-4.21 (m, 2H), 4.04-3.92 (m, 2H), 3.71-3.61 (m, 1H), 3.53-3.47 |
| | yl)carbamoyl) | | | (m, 2H), 3.44-3.40 |
| | benzo[b] | | | (m, 1H), 3.28-3.26 |
| | thiophen-5- | | | (s, 2H), 3.15 (s, 1H), |
| | yl)methyl) | | | 2.45-2.35 (m, 1H), |
| | phosphonic | | | 2.28-2.19 (m, 2H), |
| | acid | | | 2.16-2.10 (m, 2H), |
| | | | | 2.05-1.95 (m, 2H), |
| | | | | 1.92-1.85 (m, 2H), |
| | | | | 1.70-1.62 (m, 1H), |
| | | | | 1.38-1.28 (m, 1H), |
| | | | | 0.90-0.75 (m, 2H), |
| | | | | 0.03-0.02 (m, 1H) |
| 421 | ((S)-(2- (((3S,6S,7aS, 8aR,9aR)-3- (3-(4- (azetidin-1- yl)pyridin-3- yl)azetidine- 1-carbonyl)- 5-oxodeca- hydro-1H- cyclopropa[d] pyrrolo[1,2- a]azocin-6- yl)carbamoyl) benzo[b] | 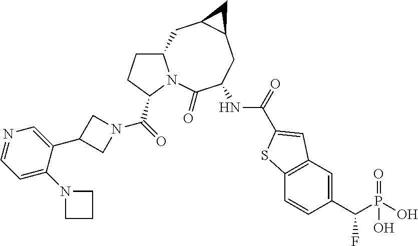 [SMILES: O=C(N[C@H]1C[C@@H]2C[C@@H]2C[C@H]2CC[C@@H](C(=O)N3CC(c4cnccc4N4CCC4)C3)N2C1=O)c1cc2cc([C@@H](F)P(=O)(O)O)ccc2s1] | 682.4 | 1H NMR (400 MHz, CD₃OD) δ 8.14 (s, 1H), 8.02-7.90 (m, 1H), 7.87-7.82 (m, 1H), 7.78-7.65 (m, 2H), 7.62-7.36 (m, 1H), 6.27-6.00 (m, 1H), 5.77-5.55 (m, 1H), 5.07-4.97 (m, 1H), 4.82-4.71 (m, 1H), 4.66-4.44 (m, 2H), 4.38-4.20 (m, 6H), 4.09-3.96 (m, 2H), 2.56-2.25 (m, 5H), 2.23-2.14 (m, |
| | thiophen-5- | | | 1H), 2.09-1.86 (m, |
| | yl)fluoro- | | | 3H), 1.83-1.67 (m, |
| | methyl) | | | 1H), 1.55-1.34 (m, |
| | phosphonic | | | 1H), 1.03-0.79 (m, |
| | acid | | | 2H), 0.15-0.10 (m, |
| | | | | 1H) |
| 420 | ((S)-(2- (((3S,6S,7aS, 8aR,9aR)-3- (3-(4- ethoxy- pyridin-3- yl)azetidine- 1-carbonyl)- 5-oxodeca- hydro-1H- cyclopropa[d] pyrrolo[1,2- a]azocin-6- yl)carbamoyl) benzo[b] | 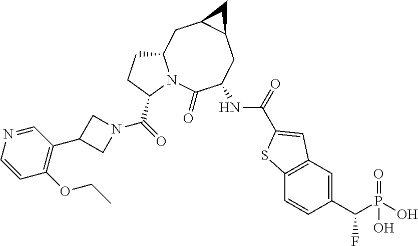 [SMILES: CCOc1ccncc1C1CN(C(=O)[C@@H]2CC[C@@H]3C[C@H]4C[C@H]4C[C@H](NC(=O)c4cc5cc([C@@H](F)P(=O)(O)O)ccc5s4)C(=O)N32)C1] | 671.1 | 1H NMR (400 MHz, CD₃OD) δ 8.41-8.23 (m, 2H), 8.04-7.88 (m, 2H), 7.86-7.74 (m, 1H), 7.61 (dd, J = 8.8, 20.0 Hz, 1H), 7.18-7.10 (m, 1H), 5.77-5.56 (m, 1H), 5.07-4.92 (m, 1H), 4.76-4.63 (m, 1H), 4.54-4.52 (m, 1H), 4.46-4.35 (m, 1H), 4.35-4.23 (m, 3H), 4.22-4.01 (m, 3H), 2.54-2.37 (m, 1H), |
| | thiophen-5- | | | 2.36-2.10 (m, 4H), |
| | yl)fluoro- | | | 2.05-1.88 (m, 2H), |
| | methyl) | | | 1.81-1.68 (m, 1H), |
| | phosphonic | | | 1.50-1.35 (m, 4H), |
| | acid | | | 1.02-0.79 (m, 2H), |
| | | | | 0.12-0.02 (m, 1H) |
| 411 | ((R)- fluoro(2- (((3S,6S,7aS, 8aR,9aR)-3- (3-(4-((S)-2- (methoxy- methyl) azetidin-1- yl)pyridin-3- yl)azetidine- 1-carbonyl)- 5-oxodeca- hydro-1H- cyclopropa[d] pyrrolo[1,2- a]azocin-6- yl)carbamoyl) benzo[b] | 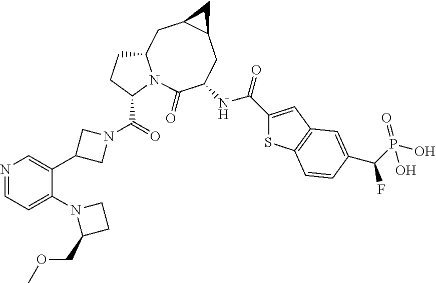 [SMILES: COC[C@@H]1CCN1c1ccncc1C1CN(C(=O)[C@@H]2CC[C@@H]3C[C@H]4C[C@H]4C[C@H](NC(=O)c4cc5cc([C@H](F)P(=O)(O)O)ccc5s4)C(=O)N32)C1] | 726.5 | 1H NMR (400 MHz, CD₃OD) δ 8.31-7.93 (m, 3H), 7.93-7.44 (m, 3H), 6.93-6.56 (m, 1H), 5.81-5.55 (m, 1H), 5.00 (t, J = 8.8 Hz, 1H), 4.82- 4.66 (m, 3H), 4.65- 4.50 (m, 2H), 4.39- 4.32 (m, 1H), 4.31- 4.26 (m, 1H), 4.14- 3.93 (m, 3H), 3.71- 3.55 (m, 2H), 3.36 (s, 3H), 2.58-2.40 (m, 2H), 2.33-2.28 (m, 1H), 2.26-2.11 (m, 3H), 2.10-1.89 (m, 3H), 1.79-1.68 (m, |
| | thiophen-5- | | | 1H), 1.46-1.30 (m, |
| | yl)methyl) | | | 1H), 0.99-0.79 (m, |
| | phosphonic | | | 2H), 0.10-0.06 (m, |
| | acid | | | 1H) |
| 410 | ((S)- fluoro(2- (((3S,6S,7aS, 8aR,9aR)-3- (3-(4-((S)-2- (methoxy- methyl) azetidin-1- yl)pyridin-3- yl)azetidine- 1-carbonyl)- 5-oxodeca- hydro-1H- cyclopropa[d] pyrrolo[1,2- a]azocin-6- | 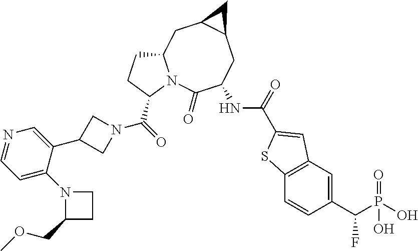 [SMILES: COC[C@@H]1CCN1c1ccncc1C1CN(C(=O)[C@@H]2CC[C@@H]3C[C@H]4C[C@H]4C[C@H](NC(=O)c4cc5cc([C@@H](F)P(=O)(O)O)ccc5s4)C(=O)N32)C1] | 726.5 | 1H NMR (400 MHz, CD₃OD) δ 8.29-7.94 (m, 3H), 7.92-7.60 (m, 3H), 6.95-6.58 (m, 1H), 5.76-5.56 (m, 1H), 5.00 (t, J = 8.8 Hz, 1H), 4.77- 4.60 (m, 3H), 4.58- 4.45 (m, 2H), 4.43- 4.35 (m, 1H), 4.30 (d, J = 8.4 Hz, 1H), 4.13- 3.92 (m, 3H), 3.72- 3.59 (m, 2H), 3.35 (d, J = 12.4 Hz, 3H), 2.58- 2.42 (m, 2H), 2.30 (d, J = 16.8 Hz, 1H), |
| | yl)carbamoyl) | | | 2.26-2.14 (m, 3H), |
| | benzo[b] | | | 2.11-1.92 (m, 3H), |
| | thiophen-5- | | | 1.78-1.72 (m, 1H), |
| | yl)methyl) | | | 1.42-1.30 (m, 1H), |
| | phosphonic | | | 0.98-0.82 (m, 2H), |
| | acid | | | 0.15-0.05 (m, 1H) |
| 408 | ((S)-(2- (((3S,6S,7aS, 8aR,9aR)-3- (3-(4- (dimethyl- amino)pyridin- 3-yl)azetidine- 1-carbonyl)- 5-oxodeca- hydro-1H- cyclopropa[d] pyrrolo[1,2- a]azocin-6- yl)carbamoyl) benzo[b] | 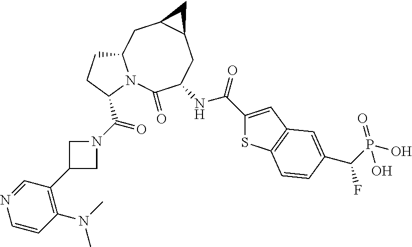 [SMILES: CN(C)c1ccncc1C1CN(C(=O)[C@@H]2CC[C@@H]3C[C@H]4C[C@H]4C[C@H](NC(=O)c4cc5cc([C@@H](F)P(=O)(O)O)ccc5s4)C(=O)N32)C1] | 670.1 | ¹HNMR (400 MHz, DMSO-d₆) δ 8.56- 8.47 (m, 1H), 8.33 (s, 1H), 8.25-8.18 (m, 2H), 8.01-7.96 (m, 2H), 7.55 (d, J = 8.6 Hz, 1H), 7.01 (d, J = 6.4 Hz, 1H), 5.84- 5.69 (m, 1H), 4.98- 4.92 (m, 1H), 4.64- 4.50 (m, 1H), 4.41 (dd, J = 4.0, 8.0 Hz, 1H), 4.34-4.13 (m, 4H), 4.02-3.92 (m, 1H), 2.98 (s, 6H), |
| | thiophen-5- | | | 2.36-2.31 (m, 1H), |
| | yl)fluoro- | | | 2.20-2.11 (m, 2H), |
| | methyl) | | | 2.10-2.00 (m, 2H), |
| | phosphonic | | | 1.92-1.84 (m, 2H), |
| | acid | | | 1.70-1.62 (m, 1H), |
| | | | | 1.42-1.34 (m, 1H), |
| | | | | 0.87-0.79 (m, 1H), |
| | | | | 0.74 (dd, J = 4.4, 8.0 |
| | | | | Hz, 1H), 0.03-0.01 |
| | | | | (m, 1H) |
| 407 | ((R)-(2- (((3S,6S,7aS, 8aR,9aR)-3- (3-(4- (dimethyl- amino)pyridin- 3-yl)azetidine- 1-carbonyl)- 5-oxodeca- hydro-1H- cyclopropa[d] pyrrolo[1,2- a]azocin-6- yl)carbamoyl) benzo[b] | 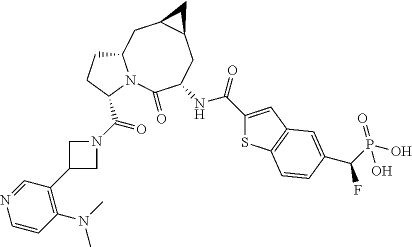 [SMILES: CN(C)c1ccncc1C1CN(C(=O)[C@@H]2CC[C@@H]3C[C@H]4C[C@H]4C[C@H](NC(=O)c4cc5cc([C@H](F)P(=O)(O)O)ccc5s4)C(=O)N32)C1] | 670.3 | 1H NMR (400 MHz, DMSO-d₆) δ 9.14- 8.70 (m, 1H), 8.33- 8.14 (m, 2H), 8.08- 7.82 (m, 3H), 7.51- 7.42 (m, 1H), 6.91- 6.64 (m, 1H), 5.72- 5.51 (m, 1H), 4.97- 4.89 (m, 1H), 4.58- 4.33 (m, 3H), 4.23- 4.15 (m, 2H), 4.06 (d, J = 7.6 Hz, 1H), 3.86 (d, J = 6.0 Hz, 1H), 2.81 (s, 6H), 2.43- 2.32 (m, 1H), 2.09 (d, |
| | thiophen-5- | | | J = 16.0 Hz, 3H), 2.03- |
| | yl)fluoro- | | | 1.94 (m, 1H), 1.88- |
| | methyl) | | | 1.77 (m, 2H), 1.69- |
| | phosphonic | | | 1.58 (m, 1H), 1.42- |
| | acid | | | 1.27 (m, 1H), 0.83- |
| | | | | 0.67 (m, 2H), −0.02 |
| | | | | (d, J = 4.0 Hz, 1H) |
| 406 | ((R)- fluoro(2- (((3S,6S,7aS, 8aR,9aR)-3- (3-(4- morpholino- pyridin-3- yl)azetidine- 1-carbonyl)- 5-oxodeca- hydro-1H- cyclopropa[d] pyrrolo[1,2- a]azocin-6- yl)carbamoyl) benzo[b] | 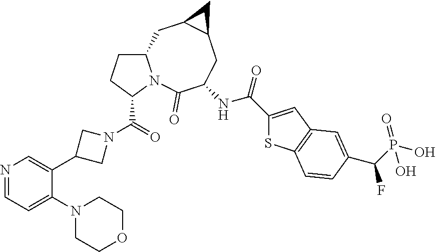 [SMILES: O=C(N[C@H]1C[C@@H]2C[C@@H]2C[C@H]2CC[C@@H](C(=O)N3CC(c4cnccc4N4CCOCC4)C3)N2C1=O)c1cc2cc([C@H](F)P(=O)(O)O)ccc2s1] | 712.1 | 1H NMR (400 MHz, CD₃OD) δ 8.44-8.32 (m, 1H), 8.18-8.10 (m, 1H), 8.06-7.92 (m, 2H), 7.86 (t, J = 8.4 Hz, 1H), 7.62- 7.55 (m, 1H), 7.24- 7.13 (m, 1H), 5.84- 5.69 (m, 1H), 5.09- 4.95 (m, 2H), 4.82- 4.69 (m, 2H), 4.66- 4.62 (m, 1H), 4.56- 4.28 (m, 4H), 4.16- 4.01 (m, 2H), 3.80- 3.75 (m, 4H), 3.35- 3.33 (m, 1H), 2.51- |
| | thiophen-5- | | | 2.39 (m, 1H), 2.34- |
| | yl)methyl) | | | 2.17 (m, 4H), 2.04- |
| | phosphonic | | | 1.94 (m, 2H), 1.79- |
| | acid | | | 1.70 (m, 1H), 1.42- |
| | | | | 1.31 (m, 1H), 0.97- |
| | | | | 0.90 (m, 1H), 0.84 (s, |
| | | | | 1H), 0.11-0.05 (m, |
| | | | | 1H) |
| 405 | ((S)- fluoro(2- (((3S,6S,7aS, 8aR,9aR)-3- (3-(4- morpholino- pyridin-3- yl)azetidine- 1-carbonyl)- 5-oxodeca- hydro-1H- cyclopropa[d] pyrrolo[1,2- a]azocin-6- yl)carbamoyl) benzo[b] thiophen-5- yl)methyl) phosphonic | 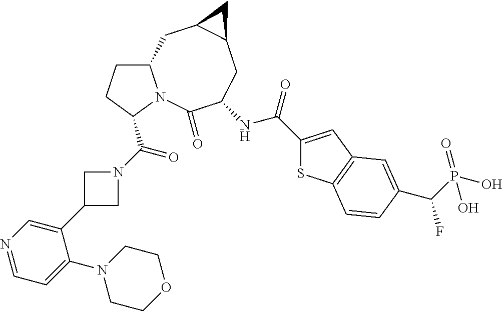 [SMILES: O=C(N[C@H]1C[C@@H]2C[C@@H]2C[C@H]2CC[C@@H](C(=O)N3CC(c4cnccc4N4CCOCC4)C3)N2C1=O)c1cc2cc([C@@H](F)P(=O)(O)O)ccc2s1] | 712.2 | 1H NMR (400 MHz, CD₃OD) δ 8.45-8.30 (m, 1H), 8.18-8.07 (m, 1H), 8.04-7.90 (m, 2H), 7.85 (dd, J = 3.2, 8.0 Hz, 1H), 7.56 (d, J = 8.0 Hz, 1H), 7.25-7.07 (m, 1H), 5.85-5.67 (m, 1H), 5.08-4.88 (m, 2H), 4.75 (t, J = 8.4 Hz, 2H), 4.66-4.51 (m, 2H), 4.31 (d, J = 6.8 Hz, 2H), 4.18-3.99 (m, 2H), 3.80-3.75 (m, 4H), 3.36-3.32 (m, 2H), 2.51-2.37 (m, 1H), 2.34-2.14 (m, 4H), 2.04-1.92 |
| | acid | | | (m, 2H), 1.81-1.67 |
| | | | | (m, 1H), 1.47-1.37 |
| | | | | (m, 1H), 0.98-0.90 |
| | | | | (m, 1H), 0.86-0.84 |
| | | | | (m, 1H), 0.10-0.06 |
| | | | | (m, 1H) |
| 404 | ((S)- fluoro(2- (((3S,6S,7aS, 8aR,9aR)-3- (3-(4-(4- methyl- piperazin-1- yl)pyridin-3- yl)azetidine- 1-carbonyl)- 5-oxodeca- hydro-1H- cyclopropa[d] pyrrolo[1,2- a]azocin-6- yl)carbamoyl) benzo[b] thiophen-5- yl)methyl) | 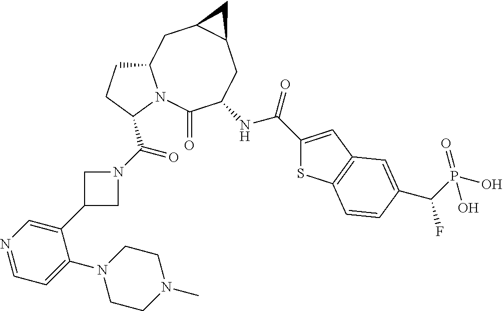 [SMILES: CN1CCN(c2ccncc2C2CN(C(=O)[C@@H]3CC[C@@H]4C[C@H]5C[C@H]5C[C@H](NC(=O)c5cc6cc([C@@H](F)P(=O)(O)O)ccc6s5)C(=O)N43)C2)CC1] | 725.2 | 1H NMR (400 MHz, CD₃OD) δ 8.38-8.25 (m, 1H), 8.15-8.00 (m, 1H), 7.87 (d, J = 19.6 Hz, 2H), 7.70- 7.56 (m, 1H), 7.47 (d, J = 2.0 Hz, 1H), 7.34- 6.83 (m, 1H), 5.81- 5.66 (m, 1H), 5.03- 4.90 (m, 3H), 4.85- 4.70 (m, 2H), 4.70- 4.44 (m, 2H), 4.44- 4.22 (m, 3H), 4.21- 3.97 (m, 2H), 2.90- 2.80 (m, 3H), 2.73 (s, 3H), 2.63-2.53 (m, 1H), 2.39-2.31 (m, 3H), 2.27-2.06 (m, 2H), 2.08-2.00 (m, |
| | phosphonic | | | 1H), 1.87-1.75 (m, |
| | acid | | | 2H), 1.48-1.38 (m, |
| | | | | 1H), 0.87 (s, 2H), |
| | | | | 0.15 (d, J = 5.2 Hz, |
| | | | | 1H) |
| 403 | ((R)- fluoro(2- (((3S,6S,7aS, 8aR,9aR)-3- (3-(4-(4- methyl- piperazin-1- yl)pyridin-3- yl)azetidine- 1-carbonyl)- 5-oxodeca- hydro-1H- cyclopropa[d] pyrrolo[1,2- a]azocin-6- yl)carbamoyl) benzo[b] thiophen-5- yl)methyl) | 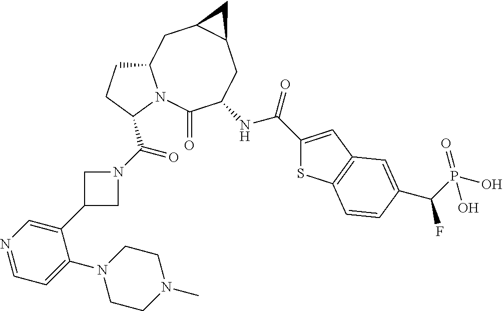 [SMILES: CN1CCN(c2ccncc2C2CN(C(=O)[C@@H]3CC[C@@H]4C[C@H]5C[C@H]5C[C@H](NC(=O)c5cc6cc([C@H](F)P(=O)(O)O)ccc6s5)C(=O)N43)C2)CC1] | 725.2 | 1H NMR (400 MHz, CD₃OD) δ 8.22-7.95 (m, 2H), 7.84-7.67 (m, 1H), 7.61 (s, 1H), 7.45 (s, 1H), 7.43- 7.39 (m, 1H), 6.64 (d, J = 6.0 Hz, 1H), 5.64- 5.50 (m, 1H), 4.87 (s, 2H), 4.60 (t, J = 7.6 Hz, 2H), 4.47 (d, J = 5.2 Hz, 2H), 4.22 (s, 3H), 4.05-3.82 (m, 2H), 3.37-3.22 (m, 1H), 3.09-3.01 (m, 1H), 2.65 (d, J = 3.6 Hz, 2H), 2.56 (s, 3H), 2.45 (s, 1H), 2.20 (s, 3H), 2.12 (d, J = 6.8 Hz, 2H), 1.91 |
| | phosphonic | | | (s, 1H), 1.65 (d, J = |
| | acid | | | 4.8 Hz, 2H), 1.27 (d, |
| | | | | J = 1.2 Hz, 1H), 0.83- |
| | | | | 0.69 (m, 2H), 0.00 |
| | | | | (s, 1H) |
| 402 | ((1R)-(2- (((3S,6S,7aS, 8aR)-3-(3-(4- (azetidin-1- yl)pyrimidin- 5- yl)azetidine- 1-carbonyl)- 5-oxodeca- hydro-1H- cyclopropa[d] pyrrolo[1,2- a]azocin-6- yl)carbamoyl) benzo[b] thiophen-5- yl)fluoro- methyl) phosphonic | 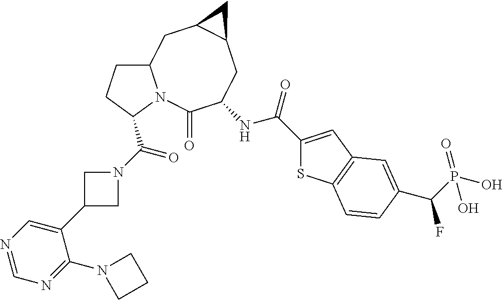 [SMILES: O=C(N[C@H]1C[C@@H]2C[C@@H]2CC2CC[C@@H](C(=O)N3CC(c4cncnc4N4CCC4)C3)N2C1=O)c1cc2cc([C@H](F)P(=O)(O)O)ccc2s1] | 683.4 | 1H NMR (400 MHz, CD₃OD) δ 8.38-8.15 (m, 1H), 8.06-7.94 (m, 3H), 8.03-7.97 (m, 1H), 7.55 (dd, J = 8.4, 14.8 Hz, 1H), 5.84-5.69 (m, 1H), 5.05-4.97 (m, 1H), 4.82-4.77 (m, 1H), 4.63-4.58 (m, 1H), 4.51-4.40 (m, 4H), 4.35-4.12 (m, 3H), 4.08-4.02 (m, 1H), 4.01-3.92 (m, 1H), 2.50-2.38 (m, 3H), 2.35-2.26 (m, 2H), 2.25-2.13 (m, 2H), 2.01-1.93 (m, 2H), 1.79-1.70 (m, 1H), |
| | acid | | | 1.49-1.37 (m, 1H), |
| | | | | 0.99-0.91 (m, 1H), |
| | | | | 0.88-0.81 (m, 1H), |
| | | | | 0.12-0.06 (m, 1H). |
| 401 | ((1S)-(2- (((3S,6S,7aS, 8aR)-3-(3-(4- (azetidin-1- yl)pyrimidin- 5- yl)azetidine- 1-carbonyl)- 5-oxodeca- hydro-1H- cyclopropa[d] pyrrolo[1,2- a]azocin-6- yl)carbamoyl) benzo[b] thiophen-5- yl)fluoro- methyl) phosphonic | 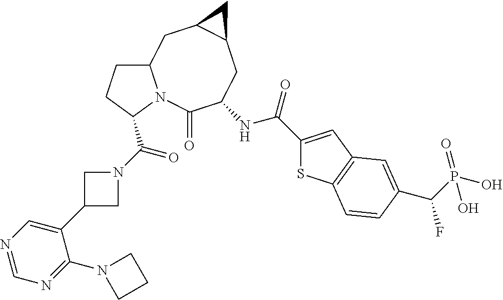 [SMILES: O=C(N[C@H]1C[C@@H]2C[C@@H]2CC2CC[C@@H](C(=O)N3CC(c4cncnc4N4CCC4)C3)N2C1=O)c1cc2cc([C@@H](F)P(=O)(O)O)ccc2s1] | 683.2 | 1H NMR (400 MHz, CD₃OD) δ 8.26-8.16 (m, 1H), 8.15-8.04 (m, 1H), 8.02-7.64 (m, 3H), 7.61-7.59 (m 1H), 5.80-5.68 (m, 1H), 5.01-4.99 (m, 1H), 4.69-4.59 (m, 2H), 4.51-4.39 (m, 5H), 4.31-4.27 (m, 2H), 4.05-4.03 (m, 2H), 2.49-2.41 (m, 3H), 2.29-2.21 (m, 4H), 2.03-1.95 (m, 2H), 1.81-1.75 (m, 1H), 1.49-1.37 (m, 1H), 0.93-0.83 (m, 2H), 0.10-0.06 (m, 1H) |
| | acid | | | |
| 429 | ((R)-(2- (((3S,6S,7aS, 8aR,9aR)-3- ((6S,7R)-7- cyano-6- (pyridin-3- yl)-4- azaspiro[2.4] heptane-4- carbonyl)-5- oxodecahydro- 1H- cyclopropa[d] pyrrolo[1,2- a]azocin-6- yl)carbamoyl) benzo[b] thiophen-5- yl)fluoro- methyl) | 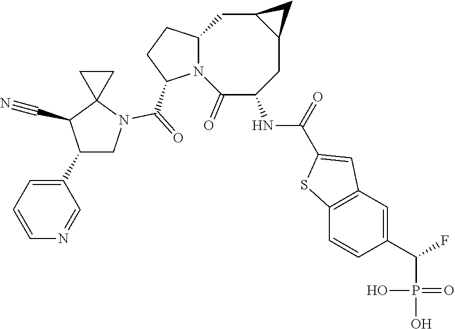 [SMILES: N#C[C@@H]1[C@@H](c2cccnc2)CN(C(=O)[C@@H]2CC[C@@H]3C[C@H]4C[C@H]4C[C@H](NC(=O)c4cc5cc([C@H](F)P(=O)(O)O)ccc5s4)C(=O)N32)C12CC2] | 692.3 | ¹H NMR (400 MHz, DMSO-d₆) δ 8.86 (d, J = 7.6 Hz, 1H), 8.78 (s, 1H), 8.66-8.57 (m, 1H), 8.28 (s, 1H), 8.14-8.06 (m, 1H), 8.03 (d, J = 8.4 Hz, 1H), 7.98 (s, 1H), 7.61-7.49 (m, 2H), 5.84 (dd, J = 44.3, 7.9 Hz, 1H), 4.98-4.90 (m, 1H), 4.60-4.51 (m, 2H), 4.17-4.12 (m, 1H), 4.06 (d, J = 9.3 Hz, 1H), 3.89- 3.78 (m, 2H), 2.37- 2.31 (m, 1H), 2.23- 1.76 (m, 8H), 1.74- 1.58 (m, 2H), 1.39 1.25 (m, 1H), 0.87- |
| | phosphonic | Or | | 0.63 (m, 4H). |
| | acid Or ((R)-(2- (((3S,6S,7aS, 8aR,9aR)-3- ((6R,7S)-7- cyano-6- (pyridin-3- yl)-4- azaspiro[2.4] heptane-4- carbonyl)-5- oxodecahydro- 1H- cyclopropa[d] pyrrolo[1,2- a]azocin-6- yl)carbamoyl) benzo[b] thiophen-5- | 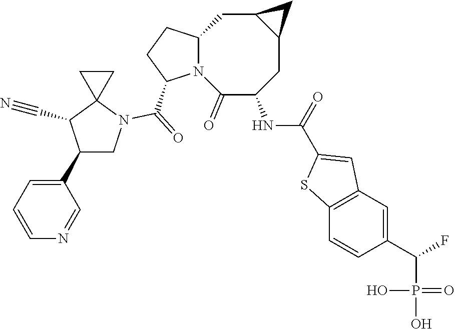 [SMILES: N#C[C@H]1[C@H](c2cccnc2)CN(C(=O)[C@@H]2CC[C@@H]3C[C@H]4C[C@H]4C[C@H](NC(=O)c4cc5cc([C@H](F)P(=O)(O)O)ccc5s4)C(=O)N32)C12CC2] | | |
| | yl)fluoro- | Pyrrolidine building block SFC | | |
| | methyl) | peak 1 was used for synthesis | | |
| | phosphonic | and biological testing | | |
| | acid | | | |
| 435 | ((R)-(2- (((3S,6S,7aS, 8aR,9aR)-3- ((S)-6-(4- (dimethyl- amino) pyridin-3- yl)-4- azaspiro[2.4] heptane-4- carbonyl)-5- oxodecahydro- 1H- cyclopropa[d] pyrrolo[1,2- a]azocin-6- yl)carbamoyl) benzo[b] thiophen-5- yl)fluoro- | 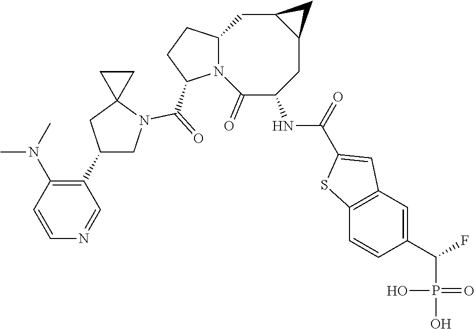 [SMILES: CN(C)c1ccncc1[C@H]1CN(C(=O)[C@@H]2CC[C@@H]3C[C@H]4C[C@H]4C[C@H](NC(=O)c4cc5cc([C@H](F)P(=O)(O)O)ccc5s4)C(=O)N32)C2(CC2)C1] | 710.2 | ¹H NMR (400 MHz, DMSO-d₆) δ 9.10 (s, 1H), 8.45-8.24 (m, 2H), 8.00 (s, 2H), 7.93-7.81 (m, 1H), 7.55-7.42 (m, 1H), 6.92-6.76 (m, 1H), 5.79-5.55 (m, 1H), 5.03-4.87 (m, 1H), 4.61-4.49 (m, 1H), 4.18-4.13 (m, 1H), 4.09-4.02 (m, 2H), 3.71-3.70 (m, 1H), 2.94 (s, 6H), 2.35- 1.59 (m, 12H), 1.40- 1.28 (m, 1H), 0.87- 0.66 (m, 2H), 0.50- 0.28 (m, 2H), 0.05- −0.05 (m, 1H). |
| | methyl) | Or | | |
| | phosphonic acid Or ((R)-(2- (((3S,6S,7aS, 8aR,9aR)-3- ((R)-6-(4- (dimethyl- amino) pyridin-3- yl)-4- azaspiro[2.4] heptane-4- carbonyl)-5- oxodecahydro- 1H- cyclopropa[d] pyrrolo[1,2- a]azocin-6- yl)carbamoyl) | 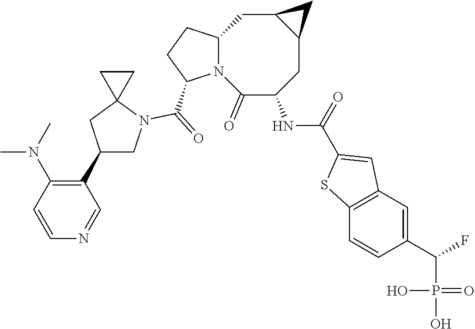 [SMILES: CN(C)c1ccncc1[C@@H]1CN(C(=O)[C@@H]2CC[C@@H]3C[C@H]4C[C@H]4C[C@H](NC(=O)c4cc5cc([C@H](F)P(=O)(O)O)ccc5s4)C(=O)N32)C2(CC2)C1] | | |
| | benzo[b] | Pyrrolidine + Core SFC peak 2 | | |
| | thiophen-5- | was used for synthesis and | | |
| | yl)fluoro- | biological testing | | |
| | methyl) | | | |
| | phosphonic | | | |
| | acid | | | |
| 433 | ((R)-(2- (((3S,6S,7aS, 8aR,9aR)-3- ((R)-6-(4- (dimethyl- amino) pyridin-3- yl)-4- azaspiro[2.4] heptane-4- carbonyl)-5- oxodecahydro- 1H- cyclopropa[d] pyrrolo[1,2- a]azocin-6- yl)carbamoyl) benzo[b] thiophen-5- yl)fluoro- | 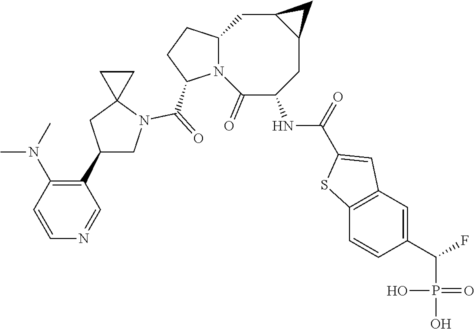 [SMILES: CN(C)c1ccncc1[C@@H]1CN(C(=O)[C@@H]2CC[C@@H]3C[C@H]4C[C@H]4C[C@H](NC(=O)c4cc5cc([C@H](F)P(=O)(O)O)ccc5s4)C(=O)N32)C2(CC2)C1] | 710.3 | ¹H NMR (400 MHz, DMSO-d₆) δ 8.90- 8.75 (m, 1H), 8.44- 8.15 (m, 3H), 8.05- 7.89 (m, 2H), 7.63- 7.45 (m, 1H), 7.20- 6.98 (m, 1H), 5.90- 5.72 (m, 1H), 5.00- 4.87 (m, 1H), 4.61- 4.43 (m, 2H), 4.18- 4.11 (m, 1H), 3.75- 3.61 (m, 2H), 3.12 (s, 6H), 2.38-2.22 (m, 2H), 2.19-1.76 (m, 8H), 1.69-1.56 (m, 2H), 1.36-1.24 (m, 1H), 0.88-0.70 (m, 2H), 0.55-0.45 (m, 2H), 0.03-0.00 (m, 1H). |
| | methyl) | Or | | |
| | phosphonic acid Or ((R)-(2- (((3S,6S,7aS, 8aR,9aR)-3- ((S)-6-(4- (dimethyl- amino) pyridin-3- yl)-4- azaspiro[2.4] heptane-4- carbonyl)-5- oxodecahydro- 1H- cyclopropa[d] pyrrolo[1,2- a]azocin-6- yl)carbamoyl) | 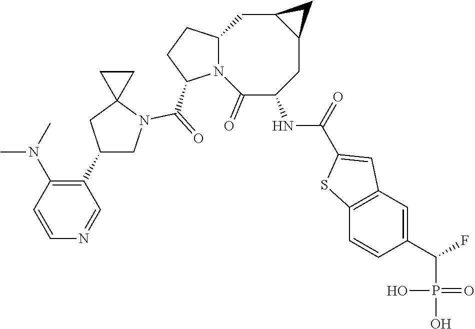 [SMILES: CN(C)c1ccncc1[C@H]1CN(C(=O)[C@@H]2CC[C@@H]3C[C@H]4C[C@H]4C[C@H](NC(=O)c4cc5cc([C@H](F)P(=O)(O)O)ccc5s4)C(=O)N32)C2(CC2)C1] | | |
| | benzo[b] | Pyrrolidine + Core SFC peak 1 | | |
| | thiophen-5- | was used for synthesis and | | |
| | yl)fluoro- | biological testing | | |
| | methyl) | | | |
| | phosphonic | | | |
| | acid | | | |
| 243 | ((2- (((3S,6S,7aR, 8aS,9aR)-3- (3-(4- methyl- pyridin-3- yl)azetidine- 1-carbonyl)- 5-oxodeca- hydro-1H- cyclopropa[d] pyrrolo[1,2- a]azocin-6- yl)carbamoyl) benzo[b] thiophen-5- yl)methyl) phosphonic | 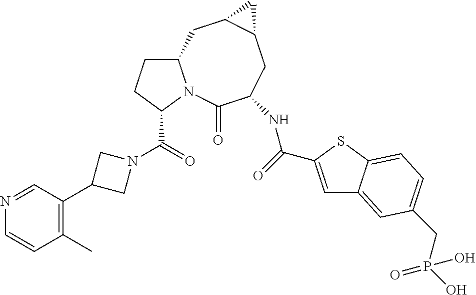 [SMILES: Cc1ccncc1C1CN(C(=O)[C@@H]2CC[C@@H]3C[C@@H]4C[C@@H]4C[C@H](NC(=O)c4cc5cc(CP(=O)(O)O)ccc5s4)C(=O)N32)C1] | 623.1 [M + H]⁺ | ¹H NMR (400 MHz, CD₃OD) δ 8.64-8.43 (m, 1H), 8.31-8.09 (m, 1H), 8.00-7.90 (m, 1H), 7.85-7.65 (m, 2H), 7.48-7.23 (m, 2H), 4.95-4.92 (m, 1H), 4.74-4.59 (m, 1H), 4.57-4.29 (m, 3H), 4.25-4.04 (m, 2H), 3.21-3.06 (m, 2H), 2.43-2.09 (m, 7H), 2.07-1.58 (m, 5H), 1.53-1.39 (m, 1H), 1.36-1.25 (m, 1H), 0.91-0.83 (m, 1H), 0.26-0.13 (m, 1H). |
| | acid | | | |
| 246 | ((2- (((3S,6S,7aS, 8aR,9aR)-3- (((3S,4S)-4- fluoro-1- (pyridin-2- yl)pyrrolidin- 3-yl) carbamoyl)-5- oxodecahydro- 1H- cyclopropa[d] pyrrolo[1,2- a]azocin-6- yl)carbamoyl) benzo[b] thiophen-5- yl)methyl) phosphonic acid | 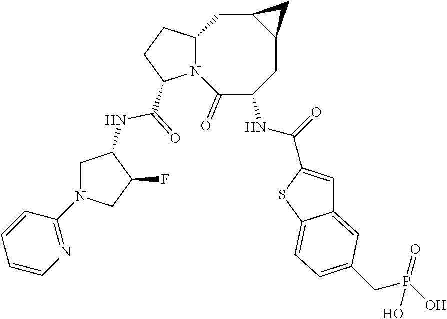 [SMILES: O=C(N[C@H]1C[C@@H]2C[C@@H]2C[C@H]2CC[C@@H](C(=O)N[C@H]3CN(c4ccccn4)C[C@@H]3F)N2C1=O)c1cc2cc(CP(=O)(O)O)ccc2s1] | 656.1 [M + H]⁺ | ¹H NMR (400 MHz, CD₃OD) δ 8.09-8.02 (m, 1H), 7.98 (s, 1H), 7.88 (br. s., 1H), 7.75- 7.68 (m, 1H), 7.61- 7.54 (m, 1H), 7.53- 7.47 (m, 1H), 6.71- 6.63 (m, 1H), 6.59- 6.52 (m, 1H), 5.26- 5.22 (m, 0.5H), 5.13- 5.09 (m, 0.5H), 5.02- 4.94 (m, 1H), 4.60- 4.54 (m, 1H), 4.53- 4.46 (m, 1H), 4.30- 4.21 (m, 1H), 3.86- 3.63 (m, 3H), 3.55- 3.46 (m, 1H), 3.12- 2.99 (m, 2H), 2.41- 2.31 (m, 1H), 2.30- 2.23 (m, 1H), 2.22- |
| | | | | 2.11 (m, 2H), 2.11- |
| | | | | 2.01 (m, 2H), 1.94- |
| | | | | 1.86 (m, 1H), 1.74- |
| | | | | 1.62 (m, 1H), 1.07- |
| | | | | 0.95 (m, 1H), 0.79- |
| | | | | 0.70 (m, 1H), 0.69- |
| | | | | 0.61 (m, 1H), 0.05- |
| | | | | −0.04 (m, 1H). |
| 256 | ((2- (((3S,6S,7aS, 8aR,9aR)-3- (((3S,4S)-4- fluoro-1- (pyridin-3- yl)pyrrolidin- 3-yl) carbamoyl)-5- oxodecahydro- 1H- cyclopropa[d] pyrrolo[1,2- a]azocin-6- yl)carbamoyl) benzo[b] thiophen-5- yl)methyl) phosphonic acid | 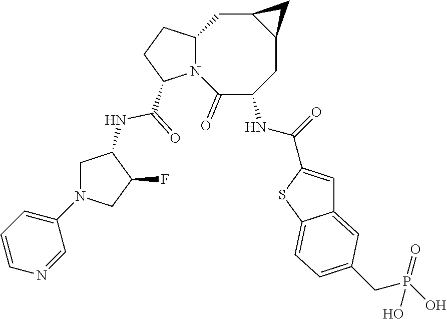 [SMILES: O=C(N[C@H]1C[C@@H]2C[C@@H]2C[C@H]2CC[C@@H](C(=O)N[C@H]3CN(c4cccnc4)C[C@@H]3F)N2C1=O)c1cc2cc(CP(=O)(O)O)ccc2s1] | 656.1 [M + H]⁺ | ¹H NMR (400 MHz, CD₃OD) δ 7.92 (s, 1H), 7.76 (m, 3H), 7.71-7.65 (m, 1H), 7.45-7.37 (m, 1H), 7.35-7.27 (m, 1H), 7.18-7.11 (m, 1H), 5.29-5.23 (m, 0.5H), 5.16-5.12 (m, 0.5H), 5.03-4.93 (m, 2H), 4.56-4.46 (m, 2H), 4.32-4.22 (m, 1H), 3.84-3.70 (m, 1H), 3.70-3.62 (m, 1H), 3.57-3.46 (m, 1H), 3.38-3.33 (m, 1H), 3.14-3.03 (m, 2H), 2.43-2.31 (m, 1H), 2.30-2.23 (m, 1H), 2.22-2.15 (m, 1H), |
| | | | | 2.13-2.07 (m, 2H), |
| | | | | 1.97-1.87 (m, 1H), |
| | | | | 1.76-1.62 (m, 1H), |
| | | | | 1.20-1.08 (m, 1H), |
| | | | | 0.89-0.78 (m, 1H), |
| | | | | 0.77-0.69 (m, 1H), |
| | | | | 0.08-0.00 (m, 1H). |
| 295 | (fluoro(2- (((3S,6S,7aS, 8aR,9aR)-5- oxo-3- ((1S,5S)-2- (pyridin-3- yl)-2,6- diazabicyclo [3.2.0] heptane-6- carbonyl) decahydro-1H- cyclopropa[d] pyrrolo[1,2- a]azocin-6- | 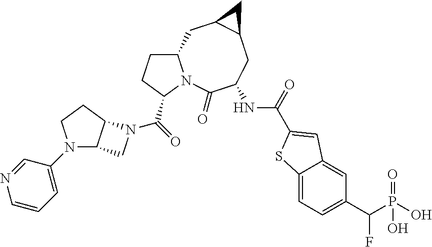 [SMILES: O=C(N[C@H]1C[C@@H]2C[C@@H]2C[C@H]2CC[C@@H](C(=O)N3C[C@H]4[C@@H]3CCN4c3cccnc3)N2C1=O)c1cc2cc(C(F)P(=O)(O)O)ccc2s1] | 668.1 [M + H]⁺ | ¹H NMR (400 MHz, CD₃OD) δ 8.18-7.79 (m, 5H), 7.67-7.61 (m, 1H), 7.34-7.22 (m, 1H), 7.22-7.12 (m, 1H), 5.76-5.55 (m, 1H), 5.32-5.08 (m, 1H), 5.06-4.97 (m, 1H), 4.74-4.62 (m, 3H), 4.44-4.38 (m, 0.7H), 4.34-4.22 (m, 1H), 4.13-4.05 (m, 0.3H), 3.94-3.86 (m, 1H), 3.77-3.69 (m, 0.6H), 3.55-3.34 |
| | yl)carbamoyl) | | | (m, 1H), 3.04-2.95 |
| | benzo[b] | | | (m, 0.4H), 2.51-2.04 |
| | thiophen-5- | | | (m, 6H), 2.03-1.80 |
| | yl)methyl) | | | (m, 2H), 1.80-1.68 |
| | phosphonic | | | (m, 1H), 1.52-1.35 |
| | acid | | | (m, 1H), 1.01-0.80 |
| | | | | (m, 2H), 0.12-0.00 |
| | | | | (m, 1H). |
| 296 | (fluoro(2- (((3S,6S,7aS, 8aR,9aR)-5- oxo-3- ((1R,5R)-2- (pyridin-3- yl)-2,6- diazabicyclo [3.2.0] heptane-6- carbonyl) decahydro-1H- cyclopropa[d] pyrrolo[1,2- a]azocin-6- yl)carbamoyl) | 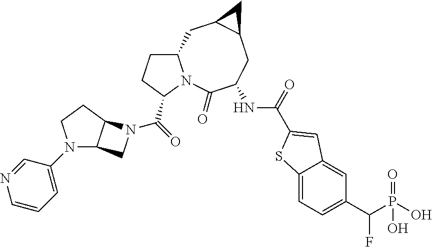 [SMILES: O=C(N[C@H]1C[C@@H]2C[C@@H]2C[C@H]2CC[C@@H](C(=O)N3C[C@@H]4[C@H]3CCN4c3cccnc3)N2C1=O)c1cc2cc(C(F)P(=O)(O)O)ccc2s1] | 668.1 [M + H]⁺ | ¹H NMR (400 MHz, CD₃OD) δ 8.13-7.79 (m, 4H), 7.75-7.48 (m, 2H), 7.32-7.12 (m, 1H), 7.03 (br. s., 1H), 5.72-5.55 (m, 1H), 5.09-4.99 (m, 2H), 4.71-4.65 (m, 1H), 4.63-4.55 (m, 1H), 4.51-4.44 (m, 1H), 4.36-4.23 (m, 1H), 4.18-4.04 (m, 1H), 3.96-3.80 (m, 1H), 3.51-3.34 (m, 1H), 2.59-2.26 (m, 4H), 2.25-2.13 (m, |
| | benzo[b] | | | 2H), 2.12-1.86 (m, |
| | thiophen-5- | | | 3H), 1.81-1.69 (m, |
| | yl)methyl) | | | 1H), 1.49-1.35 (m, |
| | phosphonic | | | 1H), 0.99-0.80 (m, |
| | acid | | | 2H), 0.12-0.02 (m, |
| | | | | 1H). |
| 257 | ((2- (((3S,6S,7aS, 8aR,9aR)-3- (((3S,4S)-4- fluoro-1- (pyrimidin-2- yl)pyrrolidin- 3-yl) carbamoyl)-5- oxodecahydro- 1H- cyclopropa[d] pyrrolo[1,2- a]azocin-6- yl)carbamoyl) benzo[b] thiophen-5- yl)methyl) phosphonic acid | 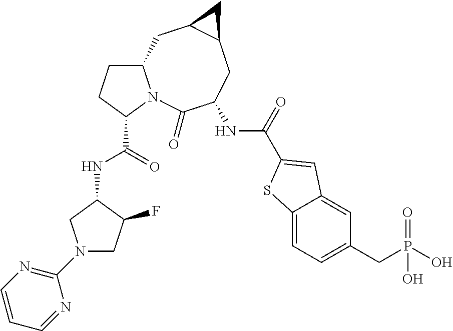 [SMILES: O=C(N[C@H]1C[C@@H]2C[C@@H]2C[C@H]2CC[C@@H](C(=O)N[C@H]3CN(c4ncccn4)C[C@@H]3F)N2C1=O)c1cc2cc(CP(=O)(O)O)ccc2s1] | 657.1 [M + H]⁺ | ¹H NMR (400 MHz, CD₃OD) δ 8.09-8.02 (m, 1H), 7.98 (s, 1H), 7.88 (br. s., 1H), 7.75- 7.68 (m, 1H), 7.61- 7.54 (m, 1H), 7.53- 7.47 (m, 1H), 6.71- 6.63 (m, 1H), 6.59- 6.52 (m, 1H), 5.26- 5.22 (m, 0.5H), 5.13- 5.09 (m, 0.5H), 5.02- 4.94 (m, 1H), 4.60- 4.54 (m, 1H), 4.53- 4.46 (m, 1H), 4.30- 4.21 (m, 1H), 3.86- 3.63 (m, 3H), 3.55- 3.46 (m, 1H), 3.12- 2.99 (m, 2H), 2.41- 2.31 (m, 1H), 2.30- 2.23 (m, 1H), 2.22- |
| | | | | 2.11 (m, 2H), 2.11- |
| | | | | 2.01 (m, 2H), 1.94- |
| | | | | 1.86 (m, 1H), 1.74- |
| | | | | 1.62 (m, 1H), 1.07- |
| | | | | 0.95 (m, 1H), 0.79- |
| | | | | 0.70 (m, 1H), 0.69- |
| | | | | 0.61 (m, 1H), 0.05- |
| | | | | −0.04 (m, 1H). |
| 258 | ((2- (((3S,6S,7aS, 8aR,9aR)-3- (6-acetyl-2,6- diazabicyclo [3.2.0]heptane- 2-carbonyl)- 5-oxodeca- hydro-1H- cyclopropa[d] pyrrolo[1,2- a]azocin-6- yl)carbamoyl) benzo[b] thiophen-5- yl)difluoro- methyl) phosphonic acid | 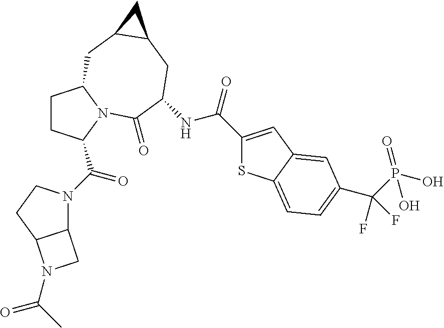 [SMILES: CC(=O)N1CC2C1CCN2C(=O)[C@@H]1CC[C@@H]2C[C@H]3C[C@H]3C[C@H](NC(=O)c3cc4cc(C(F)(F)P(=O)(O)O)ccc4s3)C(=O)N21] | 651.1 [M + H]⁺ | ¹H NMR (400 MHz, CD₃OD) δ 8.17-8.12 (m, 2H), 8.03-8.00 (m, 1H), 7.68-7.66 (m, 1H), 5.25-4.90 (m, 2H), 4.83-4.55 (m, 2H), 4.47-4.26 (m, 2H), 4.26-3.90 (m, 2H), 3.87-3.47 (m, 1H), 2.52-2.28 (m, 3H), 2.28-2.16 (m, 3H), 2.15-1.90 (m, 4H), 1.90-1.69 (m, 3H), 1.55-1.36 (m, 1H), 1.04-0.82 (m, 2H), 0.11-0.03 (m, 1H). |
| 259 | (difluoro(2- (((3S,6S,7aS, 8aR,9aR)-5- oxo-3-(6- (pyridin-3- yl)-2,6- diazabicyclo [3.2.0]heptane- 2- carbonyl) decahydro-1H- cyclopropa[d] pyrrolo[1,2- a]azocin-6- yl)carbamoyl) benzo[b] thiophen-5- yl)methyl) phosphonic acid | 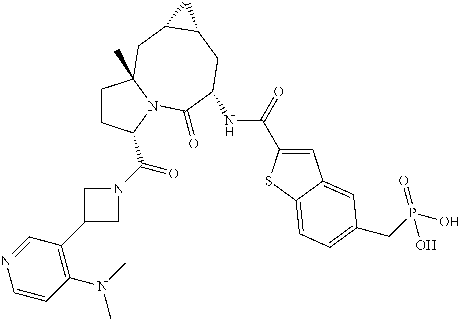 | 686.1 [M + H]⁺ | ¹H NMR (400 MHz, CD₃OD) δ 8.25-8.19 (m, 1H), 8.17-8.10 (m, 1H), 7.93-7.86 (m, 2H), 7.84-7.76 (m, 2H), 7.27-7.19 (m, 1H), 7.01-6.95 (m, 1H), 5.17-5.01 (m, 2H), 4.68-4.62 (m, 2H), 4.41-4.14 (m, 2H), 4.12-3.77 (m, 2H), 3.76-3.55 (m, 1H), 2.55-2.41 (m, 1H), 2.40-2.18 (m, 5H), 2.16-1.91 (m, 3H), 1.83-1.70 (m, 1H), 1.62-1.39 (m, 1H), 1.10-0.82 (m, 2H), 0.10-0.07 (m, 1H). |
| 260 | ((2- (((3S,6S,7aS, 8aR,9aR)-3- (6-acetyl-2,6- diazabicyclo [3.2.0]heptane- 2-carbonyl)- 5-oxodeca- hydro-1H- cyclopropa[d] pyrrolo[1,2- a]azocin-6- yl)carbamoyl) benzo[b] thiophen-5- yl)methyl) phosphonic acid | 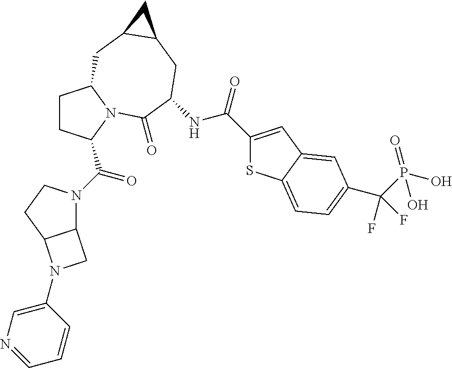 [SMILES: O=C(N[C@H]1C[C@@H]2C[C@@H]2C[C@H]2CC[C@@H](C(=O)N3CCC4C3CN4c3cccnc3)N2C1=O)c1cc2cc(C(F)(F)P(=O)(O)O)ccc2s1] | 615.1 [M + H]⁺ | ¹H NMR (400 MHz, CD₃OD) δ 8.05-8.00 (m, 1H), 7.86-7.80 (m, 2H), 7.43-7.41 (m, 1H), 5.24-4.91 (m, 2H), 4.84-4.54 (m, 2H), 4.47-4.25 (m, 2H), 4.25-3.90 (m, 2H), 3.86-3.46 (m, 1H), 3.27-3.19 (m, 2H), 2.51-2.28 (m, 3H), 2.27-2.15 (m, 3H), 2.14-1.90 (m, 4H), 1.90-1.69 (m, 3H), 1.55-1.34 (m, 1H), 1.04-0.81 (m, 2H), 0.11-0.03 (m, 1H). |
| 261 | ((2- (((3S,6S,7aS, 8aR,9aR)-5- oxo-3-(6- (pyridin-3- yl)-2,6- diazabicyclo [3.2.0]heptane- 2- carbonyl) decahydro-1H- cyclopropa[d] pyrrolo[1,2- a]azocin-6- yl)carbamoyl) benzo[b] thiophen-5- yl)methyl) phosphonic acid | 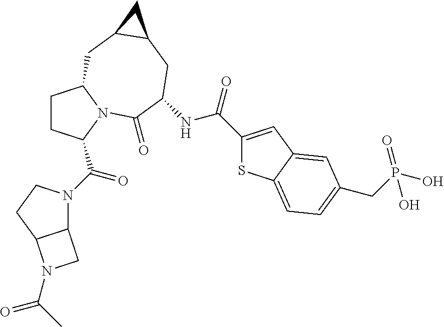 | 650.1 [M + H]⁺ | ¹H NMR (400 MHz, CD₃OD) δ 8.05-7.98 (m, 1H), 7.92-7.71 (m, 4H), 7.49-7.45 (m, 1H), 7.28-7.24 (m, 1H), 7.04-7.01 (m, 1H), 5.17-4.96 (m, 2H), 4.78-4.60 (m, 2H), 4.41-4.12 (m, 2H), 4.11-3.82 (m, 2H), 3.81-3.58 (m, 1H), 3.14-3.02 (m, 2H), 2.53-2.41 (m, 1H), 2.39-2.08 (m, 5H), 2.07-1.87 (m, 3H), 1.83-1.70 (m, 1H), 1.59-1.39 (m, 1H), 1.08-0.82 (m, 2H), 0.11-0.07 (m, 1H). |
| 270 | ((2- (((3S,6S,7aS, 8aR,9aR)-3- (2-acetyl-2,6- diazabicyclo [3.2.0]heptane- 6-carbonyl)- 5-oxodeca- hydro-1H- cyclopropa[d] pyrrolo[1,2- a]azocin-6- yl)carbamoyl) benzo[b] thiophen-5- yl)difluoro- methyl) phosphonic acid | 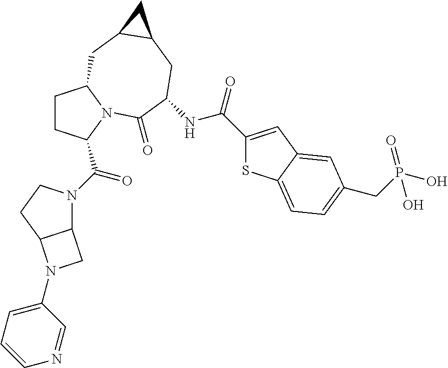 [SMILES: O=C(N[C@H]1C[C@@H]2C[C@@H]2C[C@H]2CC[C@@H](C(=O)N3CCC4C3CN4c3cccnc3)N2C1=O)c1cc2cc(CP(=O)(O)O)ccc2s1] | 651.1 [M + H]⁺ | ¹H NMR (400 MHz, CD₃OD) δ 8.16 (s, 2H), 8.01 (d, J = 8.6 Hz, 1H), 7.68 (d, J = 8.3 Hz, 1H), 5.63- 4.91 (m, 2H), 4.84- 4.61 (m, 1H), 4.60- 4.38 (m, 1H), 4.35- 4.09 (m, 2H), 4.03- 3.43 (m, 3H), 2.51- 2.40 (m, 1H), 2.39- 2.15 (m, 4H), 2.14- 1.84 (m, 7H), 1.83- 1.67 (m, 1H), 1.48- 1.33 (m, 1H), 1.00- 0.80 (m, 2H), 0.08- 0.04 (m, 1H). |
| 271 | ((2- (((3S,6S,7aS, 8aR,9aR)-3- (2-acetyl-2,6- diazabicyclo [3.2.0]heptane- 6-carbonyl)- 5-oxodeca- hydro-1H- cyclopropa[d] pyrrolo[1,2- a]azocin-6- yl)carbamoyl) benzo[b] thiophen-5- yl)methyl) phosphonic acid | 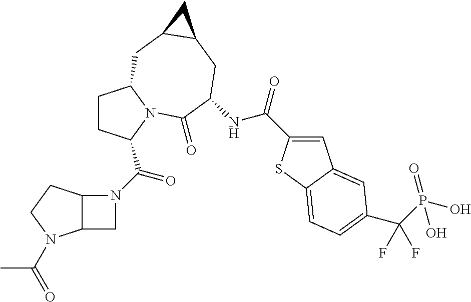 | 615.1 [M + H]⁺ | ¹H NMR (400 MHz, CD₃OD) δ 8.06-8.04 (m, 1H), 7.86-7.81 (m, 2H), 7.43 (d, J = 8.6 Hz, 1H), 5.62- 4.91 (m, 2H), 4.83- 4.61 (m, 1H), 4.60- 4.38 (m, 1H), 4.34- 4.09 (m, 2H), 4.03- 3.43 (m, 3H), 3.28- 3.20 (m, 2H), 2.49- 2.40 (m, 1H), 2.36- 2.15 (m, 4H), 2.14- 1.84 (m, 7H), 1.80- 1.66 (m, 1H), 1.52- 1.28 (m, 1H), 1.00- 0.81 (m, 2H), 0.08- 0.04 (m, 1H). |
| 275 | ((2- (((3S,6S,7aS, 8aR,9aR)-5- oxo-3-(2- (pyridin-3- yl)-2,6- diazabicyclo [3.2.0]heptane- 6-carbonyl) decahydro-1H- cyclopropa[d] pyrrolo[1,2- a]azocin-6- yl)carbamoyl) benzo[b] thiophen-5- yl)methyl) phosphonic acid | 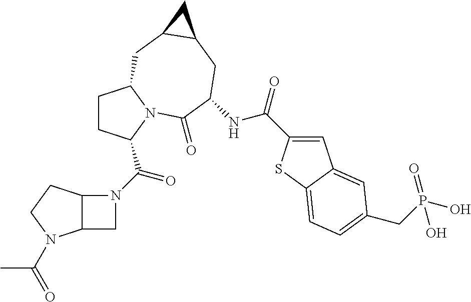 | 650.1 [M + H]⁺ | ¹H NMR (400 MHz, CD₃OD) δ 8.06-7.56 (m, 5H), 7.51-7.36 (m, 1H), 7.30-7.02 (m, 2H), 5.64-4.98 (m, 2H), 4.69-4.63 (m, 2H), 4.51-4.24 (m, 2H), 4.17-3.69 (m, 2H), 3.52-3.34 (m, 1H), 3.15-3.01 (m, 2H), 2.59-2.33 (m, 2H), 2.32-2.02 (m, 5H), 2.01-1.69 (m, 3H), 1.52-1.32 (m, 1H), 1.02-0.79 (m, 2H), 0.09-0.01 (m, 1H). |
| 274 | (difluoro(2- (((3S,6S,7aS, 8aR,9aR)-5- oxo-3-(2- (pyridin-3- yl)-2,6- diazabicyclo [3.2.0]heptane- 6-carbonyl) decahydro-1H- cyclopropa[d] pyrrolo[1,2- a]azocin-6- yl)carbamoyl) benzo[b] thiophen-5- yl)methyl) phosphonic acid | 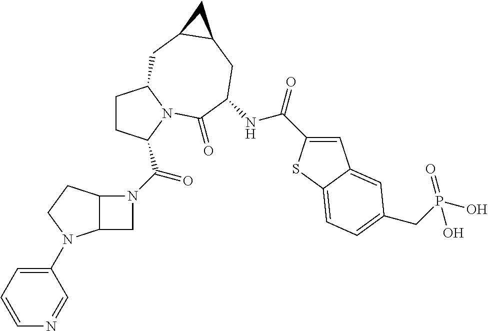 [SMILES: O=C(N[C@H]1C[C@@H]2C[C@@H]2C[C@H]2CC[C@@H](C(=O)N3CC4C3CCN4c3cccnc3)N2C1=O)c1cc2cc(CP(=O)(O)O)ccc2s1] | 686.1 [M + H]⁺ | ¹H NMR (400 MHz, CD₃OD) δ 8.24-8.03 (m, 2H), 8.02-7.85 (m, 3H), 7.82-7.65 (m, 1H), 7.31-7.03 (m, 2H), 5.65-5.02 (m, 2H), 4.69-4.60 (m, 2H), 4.49-4.38 (m, 1H), 4.36-4.23 (m, 1H), 4.18-3.71 (m, 2H), 3.53-3.35 (m, 1H), 2.57-2.35 (m, 2H), 2.34-2.04 (m, 5H), 2.03-1.69 (m, 3H), 1.54-1.35 (m, 1H), 1.03-0.79 (m, 2H), 0.13-−0.01 (m, 1H). |
| 276 | ((2- (((3S,6S,7aS, 8aR,9aR)-3- (((3S,4S)-4- fluoro- pyrrolidin-3- yl) carbamoyl)-5- oxodecahydro- 1H- cyclopropa[d] pyrrolo[1,2- a]azocin-6- yl)carbamoyl) benzo[b] thiophen-5- yl)methyl) phosphonic acid | 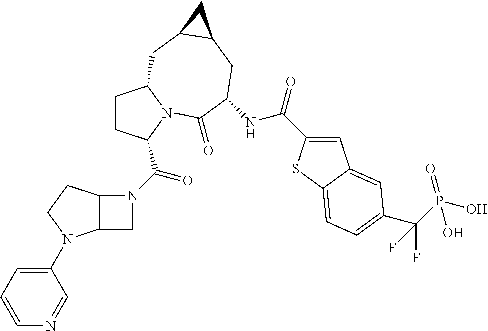 [SMILES: O=C(N[C@H]1C[C@@H]2C[C@@H]2C[C@H]2CC[C@@H](C(=O)N3CC4C3CCN4c3cccnc3)N2C1=O)c1cc2cc(C(F)(F)P(=O)(O)O)ccc2s1] | 579.1 [M + H]⁺ | ¹H NMR (400 MHz, CD₃OD) δ 8.09 (s, 1H), 7.85 (s, 1H), 7.75 (d, J = 8.3 Hz, 1H), 7.45 (d, J = 8.3 Hz, 1H), 5.11-5.00 (m, 2H), 4.57-4.51 (m, 1H), 4.33-4.16 (m, 2H), 3.30-3.15 (m, 3H), 3.14-3.04 (m, 2H), 2.73-2.62 (m, 1H), 2.51-2.41 (m, 1H), 2.34-2.23 (m, 1H), 2.22-1.89 (m, 5H), 1.80-1.68 (m, 1H), 1.28-1.16 (m, 1H), 1.01-0.83 (m, 2H), 0.13-0.05 (m, 1H). |
| 278 | ((2- (((3S,6S,7aS, 8aR,9aR)-3- (((3S,4S)-4- fluoro-1-(4- methylpyridin- 3- yl)pyrrolidin- 3-yl) carbamoyl)-5- oxodecahydro- 1H- cyclopropa[d] pyrrolo[1,2- a]azocin-6- yl)carbamoyl) benzo[b] | 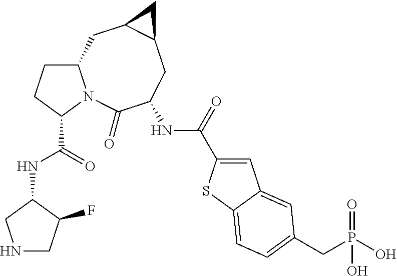 [SMILES: O=C(N[C@H]1C[C@@H]2C[C@@H]2C[C@H]2CC[C@@H](C(=O)N[C@H]3CNC[C@@H]3F)N2C1=O)c1cc2cc(CP(=O)(O)O)ccc2s1] | 670.1 [M + H]⁺ | ¹H NMR (400 MHz, CD₃OD) δ 7.90-7.83 (m, 1H), 7.77 (s, 1H), 7.74-7.65 (m, 1H), 7.61-7.55 (m, 2H), 7.32-7.26 (m, 1H), 7.21-7.16 (m, 1H), 5.13-5.09 (m, 0.5H), 5.00-4.96 (m, 0.5H), 4.93-4.85 (m, 1H), 4.46-4.35 (m, 2H), 4.24-4.16 (m, 1H), 3.98-3.92 (m, 0.5H), 3.89-3.83 (m, 0.5H), 3.77-3.70 (m, 1H), 3.56-3.24 (m, 2H), 3.15-3.09 (m, 1H), |
| | thiophen-5- | | | 3.06-2.96 (m, 2H), |
| | yl)methyl) | | | 2.39-2.32 (m, 3H), |
| | phosphonic | | | 2.32-2.22 (m, 1H), |
| | acid | | | 2.21-2.14 (m, 1H), |
| | | | | 2.13-1.99 (m, 3H), |
| | | | | 1.91-1.78 (m, 1H), |
| | | | | 1.68-1.56 (m, 1H), |
| | | | | 1.15-1.01 (m, 1H), |
| | | | | 0.82-0.77 (m, 1H), |
| | | | | 0.75-0.67 (m, 1H), |
| | | | | 0.00-−0.05 (m, 1H). |
| 290 | ((2- (((3S,6S,7aS, 8aR,9aR)-3- ((1R,5R)-2- acetyl-2,6- diazabicyclo [3.2.0]heptane- 6-carbonyl)- 5-oxodeca- hydro-1H- cyclopropa[d] pyrrolo[1,2- a]azocin-6- yl)carbamoyl) benzo[b] thiophen-5- yl)fluoro- methyl) phosphonic acid | 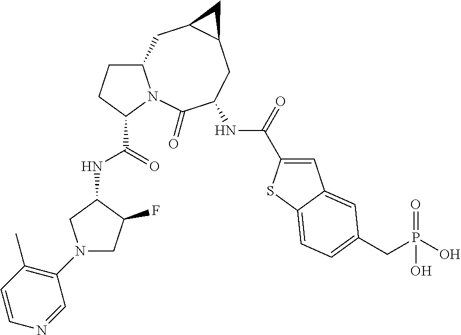 | 633.1 [M + H]⁺ | ¹H NMR (400 MHz, CD₃OD) δ 8.14-8.00 (m, 2H), 7.94 (d, J = 8.3 Hz, 1H), 7.59 (d, J = 8.6 Hz, 1H), 5.91- 5.72 (m, 1H), 5.06- 4.90 (m, 2H), 4.70- 4.60 (m, 1H), 4.55- 4.07 (m, 4H), 4.04- 3.77 (m, 1H), 3.65- 3.44 (m, 1H), 2.51- 2.07 (m, 8H), 2.06- 1.88 (m, 4H), 1.82- 1.65 (m, 1H), 1.47- 1.28 (m, 1H), 0.97- 0.80 (m, 2H), 0.08- 0.05 (m, 1H). |
| 289 | ((2- (((3S,6S,7aS, 8aR,9aR)-3- ((1S,5S)-2- acetyl-2,6- diazabicyclo [3.2.0]heptane- 6-carbonyl)- 5- oxodecahydro- 1H- cyclopropa[d] pyrrolo[1,2- a]azocin-6- yl)carbamoyl) benzo[b] thiophen-5- yl)fluoro- | 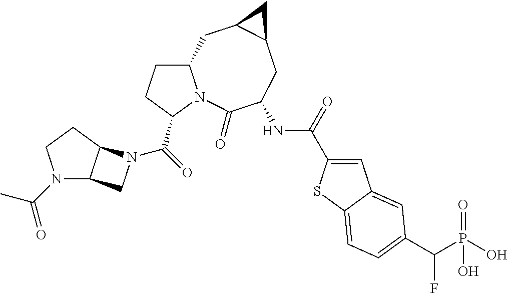 [SMILES: CC(=O)N1CC[C@@H]2[C@H]1CN2C(=O)[C@@H]1CC[C@@H]2C[C@H]3C[C@H]3C[C@H](NC(=O)c3cc4cc(C(F)P(=O)(O)O)ccc4s3)C(=O)N21] | 633.1 [M + H]⁺ | ¹H NMR (400 MHz, CD₃OD) δ 8.12-7.98 (m, 2H), 7.94 (d, J = 8.3 Hz, 1H), 7.59 (d, J = 8.8 Hz, 1H), 5.91- 5.73 (m, 1H), 5.29- 4.92 (m, 2H), 4.84- 4.54 (m, 2H), 4.45- 4.10 (m, 2H), 4.01- 3.42 (m, 3H), 2.50- 2.37 (m, 1H), 2.37- 2.07 (m, 7H), 2.06- 1.83 (m, 4H), 1.80- 1.67 (m, 1H), 1.55- 1.28 (m, 1H), 1.01- 0.78 (m, 2H), 0.08- 0.05 (m, 1H). |
| | methyl) | | | |
| | phosphonic | | | |
| | acid | | | |
| 230 | (difluoro(2- (((3S,6S,7aS, 8aR,9aR)-8a- methyl-5- oxo-3-(3- (pyridin-3- yl)azetidine- 1-carbonyl) decahydro-1H- cyclopropa[d] pyrrolo[1,2- a]azocin-6- yl)carbamoyl) benzo[b] thiophen-5- yl)methyl) phosphonic acid | 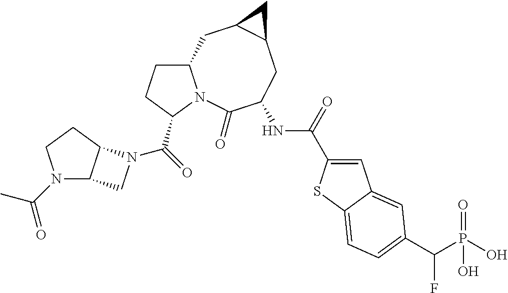 | 659.1 [M + H]⁺ | ¹H NMR (400 MHz, CD₃OD) δ 8.87-8.36 (m, 2H), 8.34-8.01 (m, 3H), 7.93 (d, J = 8.6 Hz, 1H), 7.74 (d, J = 8.6 Hz, 1H), 7.54- 7.48 (m, 1H), 5.05- 4.87 (m, 2H), 4.72- 4.65 (m, 1H), 4.57- 4.22 (m, 3H), 4.11- 3.91 (m, 2H), 2.60- 2.49 (m, 1H), 2.30- 1.91 (m, 7H), 1.32 (s, 3H), 1.1-0.96 (m, 1H), 0.60-0.57 (m, 1H), 0.39-0.29 (m, 1H). |
| 231 | (difluoro(2- (((3S,6S,7aR, 8aS,9aR)-8a- methyl-5- oxo-3-(3- (pyridin-3- yl)azetidine- 1-carbonyl) decahydro-1H- cyclopropa[d] pyrrolo[1,2- a]azocin-6- yl)carbamoyl) benzo[b] thiophen-5- yl)methyl) phosphonic acid | 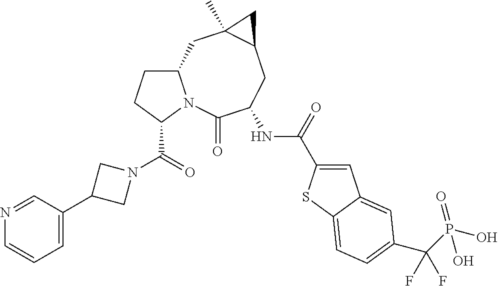 [SMILES: C[C@@]12C[C@H]3CC[C@@H](C(=O)N4CC(c5cccnc5)C4)N3C(=O)[C@@H](NC(=O)c3cc4cc(C(F)(F)P(=O)(O)O)ccc4s3)C[C@@H]1C2] | 659.1 [M + H]⁺ | ¹H NMR (400 MHz, CD₃OD) δ 8.63-8.35 (m, 2H), 8.31-8.01 (m, 3H), 7.97-7.88 (m, 1H), 7.75 (d, J = 8.6 Hz, 1H), 7.61- 7.52 (m, 1H), 5.00- 4.88 (m, 1H), 4.73- 4.68 (m, 1H), 4.62- 4.24 (m, 4H), 4.08- 3.94 (m, 2H), 2.41- 2.36 (m, 1H), 2.33- 2.15 (m, 3H), 2.05- 1.84 (m, 3H), 1.78- 1.65 (m, 1H), 1.31- 1.19 (m, 4H), 0.67- 0.64 (m, 1H), 0.39- 0.28 (m, 1H). |
| 234 | (difluoro(2- (((3S,6S,8aR, 9aS,9bR)-8a- methyl-5- oxo-3-(3- (pyridin-3- yl)azetidine- 1-carbonyl) decahydro-1H- cyclopropa[c] pyrrolo[1,2- a]azocin-6- yl)carbamoyl) | 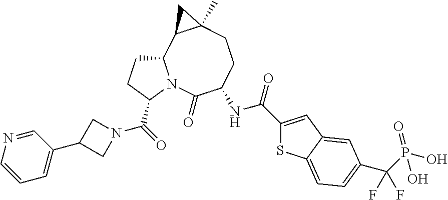 [SMILES: C[C@]12CC[C@H](NC(=O)c3cc4cc(C(F)(F)P(=O)(O)O)ccc4s3)C(=O)N3[C@H](CC[C@H]3C(=O)N3CC(c4cccnc4)C3)[C@H]1C2] | 659.1 [M + H]⁺ | ¹H NMR (400 MHz, CD₃OD) δ 8.69-8.56 (m, 1H), 8.54-8.45 (m, 1H), 8.42-8.35 (m, 1H), 8.23-8.17 (m, 1H), 8.10 (s, 1H), 7.97-7.89 (m, 1H), 7.78-7.71 (m, 1H), 7.64-7.52 (m, 1H), 5.04-4.97 (m, 0.5H), 4.74-4.68 (m, 0.5H), 4.68-4.52 (m, 2H), 4.49-4.39 (m, 1.5H), |
| | benzo[b] | | | 4.34-4.26 (m, 0.5H), |
| | thiophen-5- | | | 4.15-3.97 (m, 2H), |
| | yl)methyl) | | | 3.86-3.77 (m, 1H), |
| | phosphonic | | | 2.41-1.95 (m, 7H), |
| | acid | | | 1.77-1.66 (m, 1H), |
| | | | | 1.46-1.35 (m, 1H), |
| | | | | 1.13 (s, 3H), 0.57- |
| | | | | 0.50 (m, 1H), 0.47- |
| | | | | 0.41 (m, 1H). |
| 235 | ((2- (((3S,6S,8aR, 9aS,9bR)-8a- methyl-5- oxo-3-(3- (pyridin-3- yl)azetidine- 1-carbonyl) decahydro-1H- cyclopropa[c] pyrrolo[1,2- a]azocin-6- yl)carbamoyl) | 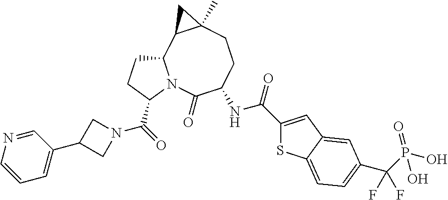 | 623.1 [M + H]⁺ | ¹H NMR (400 MHz, CD₃OD) δ 8.74-8.36 (m, 2H), 8.31-8.23 (m, 0.5H), 8.09-7.96 (m, 1.5H), 7.89-7.78 (m, 2H), 7.60-7.48 (m, 1H), 7.47-7.39 (m, 1H), 5.03-4.95 (m, 0.5H), 4.76-4.67 (m, 0.5H), 4.66-4.59 (m, 1.5H), 4.59-4.51 (m, 0.5H), 4.50-4.38 (m, 1.5H), 4.33-4.25 |
| | benzo[b] | | | (m, 0.5H), 4.14-3.96 |
| | thiophen-5- | | | (m, 2H), 3.87-3.75 |
| | yl)methyl) | | | (m, 1H), 3.28-3.17 |
| | phosphonic | | | (m, 2H), 2.40-2.23 |
| | acid | | | (m, 2H), 2.23-2.03 |
| | | | | (m, 4H), 2.02-1.93 |
| | | | | (m, 1H), 1.77-1.65 |
| | | | | (m, 1H), 1.46-1.33 |
| | | | | (m, 1H), 1.13 (s, 3H), |
| | | | | 0.57-0.50 (m, 1H), |
| | | | | 0.47-0.39 (m, 1H). |
| 236 | ((2- (((3S,6S,7aS, 8aR,9aR)-8a- methyl-5- oxo-3-(3- (pyridin-3- yl)azetidine- 1-carbonyl) decahydro-1H- cyclopropa[d] pyrrolo[1,2- a]azocin-6- | 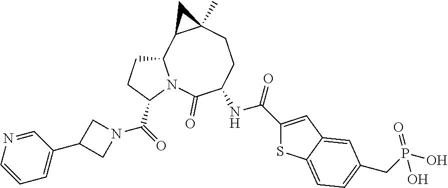 [SMILES: C[C@]12CC[C@H](NC(=O)c3cc4cc(CP(=O)(O)O)ccc4s3)C(=O)N3[C@H](CC[C@H]3C(=O)N3CC(c4cccnc4)C3)[C@H]1C2] | 623.1 [M + H]⁺ | ¹H NMR (400 MHz, CD₃OD) δ 8.58-8.22 (m, 3H), 8.08-7.99 (m, 1H), 7.86-7.77 (m, 2H), 7.57-7.38 (m, 2H), 5.04-4.87 (m, 2H), 4.71-4.60 (m, 1H), 4.55-4.36 (m, 2H), 4.32-4.23 (m, 1H), 4.09-3.91 (m, 2H), 3.26-3.14 (m, 2H), 2.59-2.48 |
| | yl)carbamoyl) | | | (m, 1H), 2.30-1.94 |
| | benzo[b] | | | (m, 7H), 1.33-1.27 |
| | thiophen-5- | | | (m, 3H), 1.03-0.92 |
| | yl)methyl) | | | (m, 1H), 0.61-0.53 |
| | phosphonic | | | (m, 1H), 0.38-0.29 |
| | acid | | | (m, 1H). |
| 237 | (difluoro(2- (((3S,6S,7aS, 8aR,9aR)-3- (3-methoxy- 3-(1-methyl- 1H-imidazol- 2- yl)azetidine- 1-carbonyl)- 8a-methyl-5- oxodecahydro- 1H- cyclopropa[d] pyrrolo[1,2- a]azocin-6- yl)carbamoyl) benzo[b] thiophen-5- | 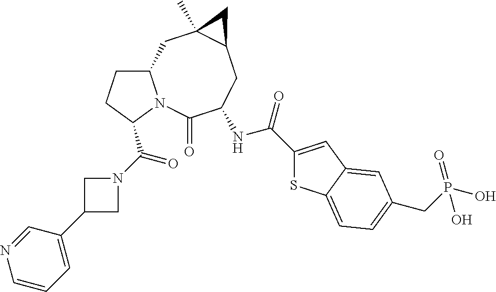 | 692.1 [M + H]⁺ | ¹H NMR (400 MHz, CD₃OD) δ 8.20- 8.04 (m, 2H), 7.95- 7.85 (m, 1H), 7.73 (d, J = 8.3 Hz, 1H), 7.24- 7.20 (m, 1H), 7.03- 7.01 (m, 1H), 5.11- 4.87 (m, 2H), 4.85- 4.79 (m, 1H), 4.67- 4.52 (m, 1H), 4.48- 4.38 (m, 1H), 4.31- 4.14 (m, 2H), 3.66 (d, J = 4.4 Hz, 3H), 3.08- 2.94 (m, 3H), 2.59- 2.41 (m, 1H), 2.30- 1.89 (m, 7H), 1.31- 1.22 (m, 3H), 1.00- 0.85 (m, 1H), 0.59- |
| | yl)methyl) | | | 0.54 (m, 1H), 0.37- |
| | phosphonic | | | 0.27 (m, 1H). |
| | acid | | | |
| 239 | (difluoro(2- (((3S,6S,7aR, 8aS,9aR)-3- (3-methoxy- 3-(1-methyl- 1H-imidazol- 2- yl)azetidine- 1-carbonyl)- 8a-methyl-5- oxodecahydro- 1H- cyclopropa[d] pyrrolo[1,2- a]azocin-6- yl)carbamoyl) benzo[b] | 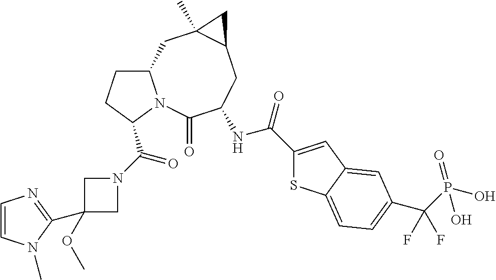 [SMILES: COC1(c2nccn2C)CN(C(=O)[C@@H]2CC[C@@H]3C[C@@]4(C)C[C@H]4C[C@H](NC(=O)c4cc5cc(C(F)(F)P(=O)(O)O)ccc5s4)C(=O)N32)C1] | 692.1 [M + H]⁺ | ¹H NMR (400 MHz, CD₃OD) δ 8.21-8.02 (m, 2H), 7.95-7.90 (m, 1H), 7.76-7.72 (m, 1H), 7.26-7.24 (m, 1H), 7.09-7.03 (m, 1H), 5.06-4.88 (m, 1H), 4.85-4.71 (m, 2H), 4.62-4.51 (m, 1H), 4.46-4.35 (m, 2H), 4.30-4.17 (m, 1H), 3.71-3.63 (m, 3H), 3.08-2.98 (m, 3H), 2.38-2.14 (m, 4H), 1.96-1.57 (m, 4H), 1.40-1.25 (m, 1H), 1.23 (s, 3H), |
| | thiophen-5- | | | 0.66-0.63 (m, 1H), |
| | yl)methyl) | | | 0.38-0.29 (m, 1H). |
| | phosphonic | | | |
| | acid | | | |
| 273 | ((2- (((3S,6S,7aR, 8aS,9aR)-3- (((3S,4S)-1- acetyl-4- fluoro- pyrrolidin-3- yl) carbamoyl)- 8a-methyl-5- oxodecahydro- 1H- cyclopropa[d] pyrrolo[1,2- a]azocin-6- yl)carbamoyl) benzo[b] | 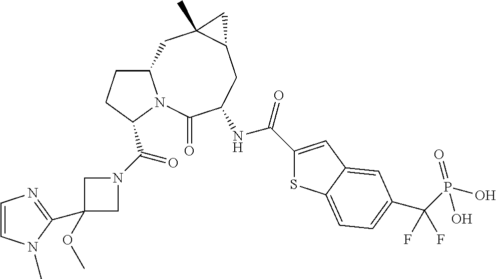 | 635.2 [M + H]⁺ | ¹H NMR (400 MHz, CD₃OD) δ 8.02-7.99 (m, 1H), 7.88-7.76 (m, 2H), 7.48-7.40 (m, 1H), 5.16-4.94 (m, 1H), 4.49-4.28 (m, 3H), 3.91-3.60 (m, 4H), 3.27-3.15 (m, 2H), 2.39-2.32 (m, 1H), 2.28-2.15 (m, 3H), 2.10-2.05 (m, 3H), 1.94-1.83 (m, 3H), 1.78-1.67 (m, 1H), 1.39-1.34 (m, 1H), 1.31-1.24 (m, 1H), 1.23 (s, 3H), 0.68-0.61 (m, 1H), |
| | thiophen-5- | | | 0.38-0.36 (m 1H). |
| | yl)methyl) | | | |
| | phosphonic | | | |
| | acid | | | |
| 241 | (difluoro(2- (((3S,6S,8aR, 9aS,9bR)-3- (3-methoxy- 3-(1-methyl- 1H-imidazol- 2- yl)azetidine- 1-carbonyl)- 8a-methyl-5- oxodecahydro- 1H- cyclopropa[c] pyrrolo[1,2- a]azocin-6- yl)carbamoyl) benzo[b] thiophen-5- yl)methyl) phosphonic | 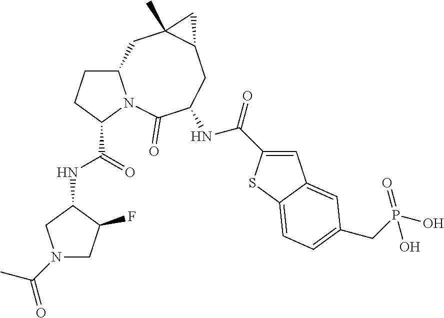 | 692.1 [M + H]⁺ | ¹H NMR (400 MHz, CD₃OD) δ 8.17 (d, J = 15.7 Hz, 1H), 8.11 (s, 0.5H), 8.02 (s, 0.5H), 7.92 (t, J = 8.7 Hz, 1H), 7.78-7.70 (m, 1H), 7.25-7.17 (m, 1H), 7.08-6.98 (m, 1H), 5.09-5.02 (m, 0.5H), 4.84-4.79 (m, 1H), 4.69-4.52 (m, 2H), 4.50-4.39 (m, 1.5H), 4.36-4.29 (m, 0.5H), 4.26-4.17 (m, 0.5H), 3.87-3.75 (m, 1H), 3.68 (d, J = 7.8 Hz, 3H), 3.05 (d, J = 10.5 Hz, 3H), 2.43- 1.88 (m, 7H), 1.78- 1.62 (m, 1H), 1.41- |
| | acid | | | 1.33 (m, 1H), 1.11 (d, |
| | | | | J = 4.9 Hz, 3H), 0.58- |
| | | | | 0.49 (m, 1H), 0.45- |
| | | | | 0.39 (m, 1H). |
| 279 | ((2- (((3S,6S,7aS, 8aR,9aR)-3- (((3S,4S)-4- fluoro-1- (pyrimidin-5- yl)pyrrolidin- 3-yl) carbamoyl)-5- oxodecahydro- 1H- cyclopropa[d] pyrrolo[1,2- a]azocin-6- yl)carbamoyl) benzo[b] thiophen-5- yl)methyl) phosphonic acid | 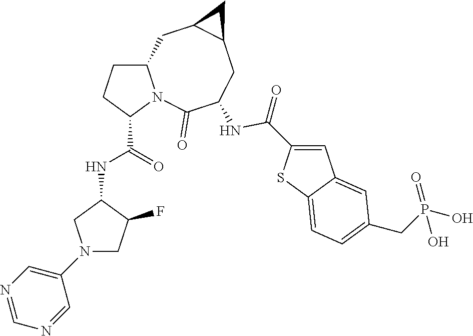 [SMILES: O=C(N[C@H]1C[C@@H]2C[C@@H]2C[C@H]2CC[C@@H](C(=O)N[C@H]3CN(c4cncnc4)C[C@@H]3F)N2C1=O)c1cc2cc(CP(=O)(O)O)ccc2s1] | 657.1 [M + H]⁺ | ¹H NMR (400 MHz, CD₃OD) δ 8.50 (s, 1H), 8.19 (s, 2H), 7.99 (s, 1H), 7.85 (br. s., 1H), 7.75 (d, J = 8.5 Hz, 1H), 7.46 (d, J = 8.5 Hz, 1H), 5.30- 5.26 (m, 0.5H), 5.17- 5.13 (m, 0.5H), 5.03- 4.95 (m, 2H), 4.57- 4.51 (m, 2H), 4.31- 4.22 (m, 1H), 3.85- 3.69 (m, 2H), 3.66- 3.55 (m, 1H), 3.46- 3.40 (m, 1H), 3.14- 3.03 (m, 2H), 2.45- 2.35 (m, 1H), 2.33- 2.24 (m, 1H), 2.23- 2.15 (m, 1H), 2.14- 2.04 (m, 2H), 1.98- 1.89 (m, 1H), 1.76- |
| | | | | 1.65 (m, 1H), 1.20 |
| | | | | 1.08 (m, 1H), 0.85- |
| | | | | 0.71 (m, 2H), 0.09- |
| | | | | 0.03 (m, 1H). |
| 419 | ((2- (((3S,6S,7aS, 8aR,9aR)-3- (3-(4- (dimethyl- amino) pyridin-3- yl)azetidine- 1-carbonyl)- 5-oxodeca- hydro-1H- cyclopropa[d] pyrrolo[1,2- a]azocin-6- yl)carbamoyl) benzo[b] thiophen-5- yl)difluoro- | 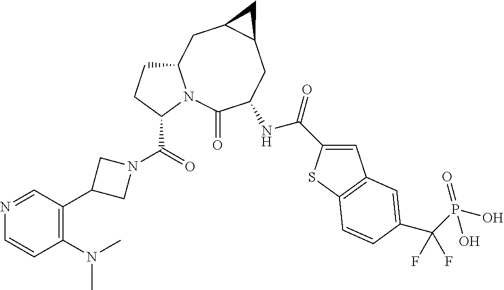 [SMILES: CN(C)c1ccncc1C1CN(C(=O)[C@@H]2CC[C@@H]3C[C@H]4C[C@H]4C[C@H](NC(=O)c4cc5cc(C(F)(F)P(=O)(O)O)ccc5s4)C(=O)N32)C1] | 688.1 [M + H]⁺ | ¹H NMR (400 MHz, CD₃OD) δ 8.44 (br. s., 1H), 8.33-8.21 (m, 1H), 8.17-8.10 (m, 1H), 8.08-7.97 (m, 1H), 7.93-7.84 (m, 1H), 7.76-7.68 (m, 1H), 6.99-6.86 (m, 1H), 5.04-4.98 (m, 1H), 4.74-4.68 (m, 1H), 4.66-4.59 (m, 1H), 4.55-4.48 (m, 1H), 4.44-4.36 (m, 1H), 4.34-4.19 (m, 2H), 4.10-3.96 (m, 1H), 3.03-2.94 (m, 6H), 2.55-2.38 (m, 1H), 2.34-2.09 |
| | methyl) | | | (m, 4H), 2.06-1.89 |
| | phosphonic | | | (m, 2H), 1.80-1.67 |
| | acid | | | (m, 1H), 1.45-1.35 |
| | | | | (m, 1H), 0.99-0.81 |
| | | | | (m, 2H), 0.12-0.02 |
| | | | | (m, 1H). |
| 238 | ((2- (((3S,6S,7aR, 8aS,9aR)-8a- methyl-5- oxo-3-(3- (pyridin-3- yl)azetidine- 1-carbonyl) decahydro-1H- cyclopropa[d] pyrrolo[1,2- a]azocin-6- yl)carbamoyl) benzo[b] thiophen-5- yl)methyl) phosphonic acid | 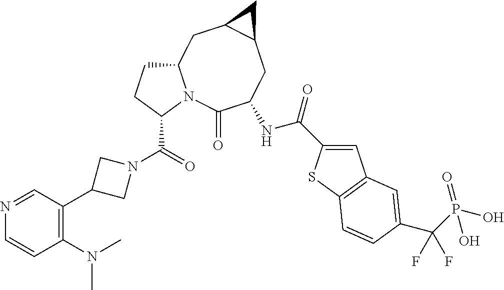 | 623.1 [M + H]⁺ | ¹H NMR (400 MHz, CD₃OD) δ 8.63-8.39 (m, 2H), 8.24-7.97 (m, 2H), 7.87-7.78 (m, 2H), 7.56-7.40 (m, 2H), 5.00-4.89 (m, 1H), 4.73-4.26 (m, 5H), 4.07-3.96 (m, 2H), 3.26-3.17 (m, 2H), 2.40-2.17 (m, 4H), 2.05-1.86 (m, 3H), 1.78-1.60 (m, 1H), 1.27-1.21 (m, 3H), 1.17-0.98 (m, 1H), 0.69-0.56 (m, 1H), 0.40-0.29 (m, 1H). |

### Biochemical and Cellular Assays

### STAT3 Fluorescence Polarization (FP) Assay

An FP assay was developed to determine IC₅₀ values for test substances. Recombinant STAT3 protein (STAT3(G127-I722)) at 25 nM was combined with a fluorescently labeled, phosphotyrosine peptide probe (5-FAM-GpYLPQTV-NH₂) at 2 nM in FP buffer (10 mM HEPES pH 7.4, 50 mM NaCl, 1 mM EDTA, 0.05% Tween 20, 2 mM DTT). 50 μL of STAT3-probe mixture was added to serial diluted compounds in black, 96-well plates (Greiner BioOne 655076) to a final concentration of 1% DMSO. Reaction components were mixed, and FP was measured after 45-minute incubation at room temperature using a Tecan Spark multimode plate reader. FP signal (mP) was plotted against the log concentration of the test substances and IC₅₀ values were calculated by nonlinear regression analysis using GraphPad Prism software. Results are shown in Table 37. For STAT3 FP assay, A=≤100 nM; B=>100-1000 nM; C=>1-10 μM; and D=>10 μM.

### STAT6 Fluorescence Polarization (FP) Assay

An FP assay was developed to determine IC₅₀ values for test substances. Recombinant STAT6 protein (STAT6(W123-T658)) at 250 nM was combined with a fluorescently labeled, phosphotyrosine peptide probe (5-FAM-ApYKPFQDLI-NH₂) at 2 nM in FP buffer (10 mM HEPES pH 7.4, 50 mM NaCl, 1 mM EDTA, 0.05% Tween 20, 2 mM DTT). 50 μL of STAT6-probe mixture was added to serial diluted compounds in black, 96-well plates (Greiner BioOne 655076) to a final concentration of 1% DMSO. Reaction components were mixed, and FP was measured after 45-minute incubation at room temperature using a Tecan Spark multimode plate reader. FP signal (mP) was plotted against the log concentration of the test substances and IC₅₀ values were calculated by nonlinear regression analysis using GraphPad Prism software. Results are shown in Table 37. For STAT6 FP assay, A=≤300 nM; B=>300-3000 nM; C=>3-30 μM; and D=>30 μM.

TABLE 37

| Cmp # | STAT3 | STAT6 |
|---|---|---|
| 1 | A | C |
| 3 | B | B |
| 7 | B | B |
| 8 | B | C |
| 9 | B | B |
| 10 | B | A |
| 11 | B | A |
| 12 | A | C |
| 13 | A | C |
| 14 | A | C |
| 15 | A | C |
| 16 | B | C |
| 17 | B | C |
| 18 | A | C |
| 19 | A | C |
| 20 | C | D |
| 21 | A | C |
| 22 | B | D |
| 23 | B | D |
| 24 | B | D |
| 25 | B | D |
| 26 | A | B |
| 27 | B | B |
| 28 | A | A |
| 29 | B | C |
| 30 | A | C |
| 31 | A | C |
| 32 | A | B |
| 33 | A | C |
| 34 | B | D |
| 35 | B | D |
| 36 | A | B |
| 37 | A | C |
| 38 | B | C |
| 39 | A | C |
| 40 | A | B |
| 41 | A | B |
| 42 | B | B |
| 43 | B | C |
| 44 | A | B |
| 45 | A | C |
| 46 | A | B |
| 47 | A | B |
| 48 | B | C |
| 49 | A | C |
| 50 | A | C |
| 51 | A | B |
| 52 | B | B |
| 53 | B | C |
| 54 | A | B |
| 55 | A | C |
| 56 | A | B |
| 57 | B | C |
| 58 | A | C |
| 59 | A | C |
| 60 | A | B |
| 61 | A | C |
| 62 | A | B |
| 63 | A | C |
| 64 | A | B |
| 65 | A | C |
| 66 | C | A |
| 67 | A | B |
| 68 | A | B |
| 69 | A | C |
| 70 | A | B |
| 71 | A | C |
| 72 | A | C |
| 73 | A | C |
| 74 | A | B |
| 75 | A | C |
| 76 | A | C |
| 77 | A | C |
| 78 | A | C |
| 79 | A | C |
| 80 | A | C |
| 81 | A | B |
| 82 | A | B |
| 83 | A | C |
| 84 | A | C |
| 85 | A | B |
| 86 | A | C |
| 87 | A | C |
| 88 | A | B |
| 89 | A | B |
| 90 | A | B |
| 91 | B | C |
| 92 | B | C |
| 93 | B | C |
| 94 | A | C |
| 95 | B | C |
| 96 | B | C |
| 97 | A | C |
| 98 | B | C |
| 99 | B | C |
| 100 | A | C |
| 101 | B | B |
| 102 | A | C |
| 103 | B | B |
| 104 | A | B |
| 105 | B | B |
| 106 | A | B |
| 107 | A | C |
| 108 | A | C |
| 109 | A | C |
| 110 | B | C |
| 111 | A | B |
| 112 | A | B |
| 113 | B | C |
| 114 | A | C |
| 115 | B | B |
| 116 | A | B |
| 117 | A | B |
| 118 | A | B |
| 119 | B | C |
| 120 | B | B |
| 121 | A | B |
| 122 | B | C |
| 123 | B | C |
| 124 | A | B |
| 125 | B | C |
| 126 | A | B |
| 127 | A | C |
| 128 | A | B |
| 129 | A | C |
| 230 | A | B |
| 231 | A | C |
| 234 | A | C |
| 235 | B | D |
| 236 | B | D |
| 237 | B | C |
| 238 | B | D |
| 239 | A | C |
| 241 | B | C |
| 243 | A | D |
| 244 | C | C |
| 246 | B | C |
| 247 | A | D |
| 248 | A | B |
| 250 | A | C |
| 252 | A | D |
| 253 | A | C |
| 254 | B | B |
| 256 | B | C |
| 257 | D | C |
| 258 | A | B |
| 259 | A | B |
| 260 | C | D |
| 261 | B | C |
| 262 | A | B |
| 265 | A | D |
| 266 | A | C |
| 267 | A | B |
| 270 | A | B |
| 271 | C | D |
| 272 | A | C |
| 273 | C | C |
| 274 | A | B |
| 275 | B | C |
| 276 | C | D |
| 277 | B | D |
| 278 | B | D |
| 279 | D | D |
| 280 | A | C |
| 282 | A | C |
| 286 | A | C |
| 288 | A | C |
| 289 | B | C |
| 290 | B | C |
| 293 | A | B |
| 294 | A | C |
| 295 | A | B |
| 296 | A | B |
| 299 | A | B |
| 300 | A | B |
| 301 | B | C |
| 302 | B | C |
| 303 | A | B |
| 304 | A | C |
| 305 | B | C |
| 312 | A | C |
| 313 | A | B |
| 317 | A | C |
| 318 | A | C |
| 320 | A | C |
| 322 | A | C |
| 324 | B | C |
| 326 | B | C |
| 327 | B | C |
| 328 | B | C |
| 330 | A | B |
| 331 | A | C |
| 333 | A | C |
| 334 | C | D |
| 335 | C | D |
| 336 | B | C |
| 337 | A | C |
| 339 | A | B |
| 340 | A | C |
| 341 | A | C |
| 342 | C | D |
| 343 | A | D |
| 344 | A | C |
| 345 | A | C |
| 346 | A | C |
| 347 | B | C |
| 348 | A | C |
| 349 | B | C |
| 350 | B | C |
| 351 | B | C |
| 352 | B | D |
| 353 | A | C |
| 354 | A | D |
| 355 | A | B |
| 356 | A | C |
| 357 | A | C |
| 358 | B | C |
| 364 | C | D |
| 365 | C | C |
| 366 | B | C |
| 367 | A | C |
| 368 | C | C |
| 369 | B | C |
| 370 | A | C |
| 371 | A | C |
| 372 | C | C |
| 373 | C | C |
| 375 | B | C |
| 376 | A | C |
| 377 | B | C |
| 378 | C | C |
| 379 | C | C |
| 380 | B | C |
| 383 | C | B |
| 384 | C | C |
| 385 | C | C |
| 386 | C | B |
| 387 | C | C |
| 388 | C | C |
| 389 | C | C |
| 390 | B | C |
| 391 | A | D |
| 393 | A | D |
| 394 | A | C |
| 400 | B | C |
| 401 | A | C |
| 402 | A | B |
| 403 | A | C |
| 404 | A | D |
| 405 | A | C |
| 406 | A | C |
| 407 | A | C |
| 408 | A | C |
| 429 | C | A |
| 410 | C | C |
| 411 | B | B |
| 419 | A | B |
| 420 | A | C |
| 421 | A | C |
| 422 | A | C |
| 425 | A | D |
| 433 | A | B |
| 435 | A | A |
| 427 | B | B |
| 428 | A | C |

### MSD-pSTAT-PBMC Assay

### Materials:

Cryopreserved Peripheral Blood Mononuclear Cells (PBMC) are from AllCells. Recombinant Human IL-4 and IL-6 are from Peprotech. Mouse monoclonal anti-STAT3 antibody, rabbit monoclonal anti-pY705-STAT3 antibody, rabbit monoclonal Anti-pY641-STAT6 antibody, and lysis buffer are from Cell Signaling Technology (CST). Mouse monoclonal anti-STAT6 antibody is from BioLegend. Assay plates, blocker, and anti-rabbit secondary antibody are from Meso Scale Discovery (MSD).

### Assay Method:

Cryopreserved PBMCs were thawed out and allowed to recover overnight in IMDM+10% heat-inactivated FBS prior to plating 50,000 (STAT3) or 25,000 (STAT6) cells per well in 96-well U-bottom tissue culture plates. Cells were treated with compound for 3 hrs, then stimulated with 10 ng/mL IL-6 (STAT3) or 1 ng/mL IL-4 (STAT6) for 10 min. Cells were then spun down and washed with ice-cold PBS prior to lysing the cell pellet with 1× lysis buffer (CST) with 1× HALT protease and phosphatase inhibitor cocktail (Thermo). Lysates were transferred to and incubated overnight at 4° C. with shaking in QuickPlex 96-well high bind assay plates (MSD) pre-coated overnight with 30 μL per well of 0.6 μg/mL mouse monoclonal anti-STAT3 antibody (STAT3) or 2 μg/mL mouse monoclonal anti-STAT6 antibody (STAT6) in 1×PBS, and pre-blocked for 1 hour with 3% Blocker-A (MSD). Captured protein in the assay plates were then washed and probed with 25 μL per well of 0.25 μg/ml rabbit monoclonal anti-pY705-STAT3 antibody (STAT3) or 0.18 μg/ml rabbit monoclonal Anti-pY641-STAT6 antibody (STAT6) in 1% Blocker-A for 1 hour at room temperature with shaking, then washed and probed with 25 ul per well of 1 ug/mi Sulfo-TAG Labeled Goat Anti-Rabbit Antibody (MSD) in 1% Blocker-A for 1 hour at room temperature with shaking. Assay plates were then washed and 150 μL of 1× Read Buffer-T (MSD) was added to each well prior to reading on an SQ120 MSD Plate Reader. Assay signal from each sample was subtracted by the signal from unstimulated control wells and normalized to DMSO control wells. IC₅₀ values were calculated using Graph Pad Prism Dose-Response Nonlinear Regression with variable slope. Results are shown in Table 38. For both pSTAT3 and pSTAT6 A=≤100 nM; B=>100-1000 nM; C=>1000-3000 nM; and D=>3 μM.

TABLE 38

| Cmp # | pSTAT3 | pSTAT6 |
|---|---|---|
| 2 | D | D |
| 4 | A | D |
| 5 | C | |
| 6 | C | |
| 130 | D | |
| 131 | B | D |
| 132 | D | |
| 133 | D | D |
| 134 | B | |
| 135 | A | D |
| 136 | C | D |
| 137 | C | D |
| 138 | A | C |
| 139 | A | A |
| 140 | A | A |
| 141 | A | C |
| 142 | A | C |
| 143 | B | D |
| 144 | B | D |
| 145 | A | C |
| 146 | D | D |
| 147 | D | |
| 148 | B | D |
| 149 | C | D |
| 150 | A | D |
| 151 | B | D |
| 152 | A | B |
| 153 | B | |
| 154 | B | D |
| 155 | A | C |
| 156 | A | D |
| 157 | A | D |
| 158 | B | D |
| 159 | D | |
| 160 | D | |
| 161 | A | D |
| 162 | B | D |
| 163 | C | |
| 164 | D | |
| 165 | C | D |
| 166 | A | D |
| 167 | D | |
| 168 | A | D |
| 169 | B | D |
| 170 | C | D |
| 171 | A | D |
| 172 | B | D |
| 173 | C | D |
| 174 | C | |
| 175 | B | D |
| 176 | A | D |
| 177 | D | D |
| 178 | A | B |
| 179 | A | D |
| 180 | A | D |
| 181 | A | D |
| 182 | A | D |
| 183 | A | B |
| 184 | D | D |
| 185 | B | D |
| 186 | D | D |
| 187 | A | B |
| 188 | A | B |
| 189 | A | C |
| 191 | B | |
| 192 | D | |
| 193 | A | D |
| 194 | B | |
| 195 | B | D |
| 196 | A | D |
| 198 | A | C |
| 200 | B | |
| 201 | A | |
| 202 | A | D |
| 203 | A | B |
| 204 | A | B |
| 205 | A | B |
| 206 | A | B |
| 207 | A | D |
| 208 | A | C |
| 209 | A | B |
| 210 | A | B |
| 211 | B | D |
| 212 | A | D |
| 213 | B | D |
| 214 | A | D |
| 215 | A | B |
| 216 | B | |
| 217 | B | |
| 218 | A | D |
| 219 | A | B |
| 220 | A | D |
| 221 | A | |
| 222 | A | D |
| 223 | B | D |
| 224 | A | D |
| 225 | A | C |
| 226 | A | |
| 227 | A | |
| 228 | B | D |
| 229 | A | |
| 232 | A | |
| 233 | A | |
| 240 | A | |
| 242 | A | |
| 245 | C | |
| 249 | A | |
| 251 | A | |
| 255 | B | |
| 263 | A | |
| 264 | A | |
| 268 | A | |
| 269 | A | |
| 281 | A | |
| 283 | A | |
| 284 | A | |
| 285 | A | |
| 287 | A | |
| 291 | A | |
| 292 | A | |
| 297 | A | |
| 298 | A | |
| 306 | A | |
| 307 | A | |
| 308 | A | |
| 309 | D | |
| 310 | D | |
| 311 | A | |
| 314 | A | |
| 315 | B | |
| 316 | A | |
| 319 | A | |
| 321 | A | |
| 323 | A | |
| 325 | B | |
| 329 | A | |
| 332 | D | |
| 338 | A | |
| 359 | A | |
| 360 | A | |
| 361 | A | |
| 362 | A | |
| 363 | B | |
| 374 | A | |
| 381 | A | |
| 382 | B | |
| 392 | A | |
| 395 | A | |
| 396 | A | |
| 397 | A | |
| 398 | A | |
| 399 | A | |
| 409 | A | |
| 412 | A | |
| 413 | A | |
| 414 | A | |
| 415 | A | |
| 430 | D | |
| 431 | D | |
| 416 | A | |
| 417 | A | |
| 418 | A | |
| 423 | A | |
| 424 | B | |
| 426 | A | |
| 432 | A | |
| 434 | A | |

While we have described a number of embodiments, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A compound having the structural formula I: 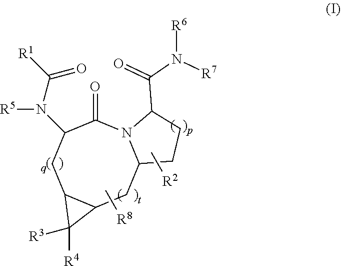
or a pharmaceutically acceptable salt thereof, wherein:
q is 0 or 1 and t is 0, 1, or 2, provided that at least one of q or t is 1;
p is 1 or 2;
R¹ is selected from an 8- to 10-membered fused bicyclic heteroaryl substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}]; an 8- to 10-membered fused bicyclic heterocyclyl substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}]; an aryl substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}]; a —(C₁-C₄)alkyl(aryl) wherein said aryl portion of —(C₁-C₄)alkyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], or —CR^{1a}R^{2a}P(O)[NH(AA)C(O)OR^{T}]][NH(AA)C(O)OR^{T}], —P(O)[OR^{1b}][NH(AA)C(O)OR^{T}]; and a —(C₂-C₄)alkenyl(aryl) wherein said aryl portion of —(C₂-C₄)alkenyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}];
R^{1a} and R^{2a} are each independently selected from hydrogen, cyano, (C₁-C₄)alkyl, hydroxy(C₁-C₄)alkyl and fluoro; or R^{1a} and R^{2a} taken together with the carbon they are attached form oxo;
R^{1b} and R^{2b} are each independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkyl]-O—[(C₁-C₂₀)alkyl], —[(C₁-C₄)alkyl]-OC(O)-[halo(C₁-C₄)alkyl], [(C₁-C₄)alkyl]-OC(O)O-[5- to 7-membered heterocyclyl], [(C₁-C₄)alkyl]-OC(O)-[5- to 7-membered heterocyclyl], —[(C₁-C₄)alkyl]-OC(O)—[(C₁-C₄)alkyl]-OH, —[(C₁-C₄)alkyl]-OC(O)—[(C₁-C₄)alkyl]-O—[(C₁-C₄)alkyl], —[(C₁-C₄)alkyl]-OC(O)O—[(C₁-C₄)alkyl], —[(C₁-C₄)alkyl]-OC(O)O-[halo(C₁-C₄)alkyl], —[(C₁-C₄)alkyl]-OC(O)O—[(C₁-C₄)alkyl]-OH, —[(C₁-C₄)alkyl]-OC(O)O—[(C₁-C₄)alkyl]-O—[(C₁-C₄)alkyl], —[(C₁-C₄)alkylphenyl]-C(O)O—[(C₁-C₄)alkyl], —[(C₁-C₄)alkyl]-OC(O)—[NH(AA)C(O)OR^{T}], —[(C₁-C₄)alkyl]-SC(O)—[(C₁-C₄)alkyl], —[(C₁-C₄)alkyl]-SC(O)-[halo(C₁-C₄)alkyl], —[(C₁-C₄)alkyl]-SC(O)—[(C₁-C₄)alkyl]-OH, —[(C₁-C₄)alkyl]-SC(O)—[(C₁-C₄)alkyl]-O—[(C₁-C₄)alkyl], —[(C₁-C₄)alkyl]-OC(O)NH(C₁-C₄)alkyl], —[(C₁-C₄)alkyl]-OC(O)N[(C₁-C₄)alkyl]₂, and aryl, wherein said 5- to 6-membered heteroaryl and aryl are each optionally and independently substituted with, as valency permits, 1 to 2 groups selected from halo, cyano, and (C₁-C₄)alkyl and wherein said 5- to 7-membered heterocyclyl of [(C₁-C₄)alkyl]-OC(O)O-[5- to 7-membered heterocyclyl] and [(C₁-C₄)alkyl]-OC(O)-[5- to 7-membered heterocyclyl] are each optionally and independently substituted with, as valency permits 1 to 2 groups selected from C(O)OR^{h};
R² is selected from hydrogen, halo, (C₁-C₄)alkyl, halo(C₁-C₄)alkyl, (C₁-C₄)alkoxy, halo(C₁-C₄)alkoxy, hydroxy(C₁-C₄)alkyl, cyano, and hydroxyl;
R³ and R⁴ are each independently selected from hydrogen, halo, and (C₁-C₄)alkyl;
R⁵ and R⁶ are each independently selected from hydrogen, phenyl, and (C₁-C₄)alkyl;
R⁷ is selected from (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, 1 to 3 groups selected from R^{Q};
R⁸ is hydrogen or (C₁-C₄)alkyl;
AA is the residue of an alpha or beta natural or non-natural amino acid;
R^{T} and R^{Ty} are each independently selected from (C₁-C₄)alkyl, (C₁-C₄)alkyl-C(O)O(C₁-C₄)alkyl, benzyl, and phenyl, wherein said phenyl is optionally substituted with 1 or 2 groups selected from halo, (C₁-C₄)alkyl, and halo(C₁-C₄)alkyl;
R^{Q} is selected from halo, (C₂-C₄)alkenyl, (C₁-C₄)alkyl, (C₁-C₄)alkoxy, halo(C₁-C₄)alkoxy, cyano, phenyl, hydroxyl, 4- to 9-membered monocyclic or bicyclic heterocyclyl, 5- to 10-membered monocyclic or bicyclic heteroaryl, (C₃-C₆)cycloalkyl, oxo, imino, —OR^{e}, —C(O)R^{g}, —C(O)OR^{e}, —NR^{c}C(O)R^{e}, —C(O)NR^{c}R^{d}, —NR^{a}R^{b}, —S(O)R^{e}R^{f}, —S(O)₂R^{f}, —S(O)═NH(C₁-C₄)alkyl, —S(O)NR^{e}R^{f}, and —S(O)₂NR^{e}R^{f}, wherein said (C₂-C₄)alkenyl and (C₁-C₄)alkyl are each optionally and independently substituted with, as valency permits, 1 to 3 groups selected from R^{M}, and wherein said phenyl, 5- to 10-membered monocyclic or bicyclic heteroaryl, (C₃-C₆)cycloalkyl, and 4- to 9-membered monocyclic or bicyclic heterocyclyl are each optionally and independently substituted with, as valency permits, 1 to 3 groups selected from R^{F};
R^{Y} is selected from halo, (C₁-C₄)alkoxy, halo(C₁-C₄)alkoxy, cyano, —C(O)R^{g}, —C(O)OR^{e}, —NHC(O)R^{e}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{M} and R^{J} are each independently selected from halo, (C₁-C₄)alkyl, (C₁-C₄)alkoxy, halo(C₁-C₄)alkoxy, cyano, —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R^{f}, —S(O)NR^{e}R^{f}, —S(O)=NR^{e}(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{F}, R^{X}, and R^{Z} are each independently selected from halo, cyano, (C₁-C₄)alkyl, cyano(C₁-C₄)alkyl, (C₃-C₆)cycloalkyl, halo(C₁-C₄)alkyl, —(C₁-C₄)alkylC(O)NR^{c}R^{d}, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, 5- to 6-membered monocyclic heteroaryl —S(O)R^{e}R^{f}, —S(O)₂R^{f}, —S(O)═NH(C₁-C₄)alkyl, —S(O)NR^{e}R^{f}, —S(O)₂NR^{e}R^{f}, —C(O)OR^{e}, —NR^{c}C(O)R^{e}, —(C₁-C₄alkyl)C(O)R^{g}, —C(O)R^{g}, —C(O)NR^{c}R^{d}, NO₂, and —NR^{a}R^{b}, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, —(C₁-C₄)alkyl(C₁-C₄)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo; and
R^{a}, R^{b}, R^{c}, R^{d}, R^{e}, R^{f}, R^{g}, and R^{h} are each independently selected from, as valency permits, hydrogen, (C₁-C₄)alkyl, (C₂-C₄)alkynyl, —(C₁-C₄)alkylphenyl, phenyl, (C₃-C₆)cycloalkyl, 4- to 6-membered heterocyclyl and 5- to 6-membered heteroaryl, wherein said (C₁-C₄)alkyl is optionally substituted with, as valency permits, 1 to 3 groups selected from R^{J}, and said phenyl, (C₃-C₆)cycloalkyl, 4- to 6-membered heterocyclyl, and 5- to 6-membered heteroaryl are each independently optionally substituted with, as valency permits, 1 to 3 groups selected from halo, cyano, (C₁-C₄)alkyl, halo(C₁-C₄)alkyl, (C₁-C₄)alkoxy, halo(C₁-C₄)alkoxy, hydroxyl, phenyl, and benzyl.

2. The compound of claim 1, or a pharmaceutically acceptable salt thereof, wherein R^{F}, R^{X}, and R^{Z} are each independently selected from halo, cyano, (C₁-C₄)alkyl, cyano(C₁-C₄)alkyl, (C₃-C₆)cycloalkyl, halo(C₁-C₄)alkyl, —(C₁-C₄)alkylC(O)NR^{c}R^{d}, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, 5- to 6-membered monocyclic heteroaryl —S(O)R^{e}R^{f}, —S(O)₂R^{f}, —S(O)═NH(C₁-C₄)alkyl, —S(O)NR^{e}R^{f}, —S(O)₂NR^{e}R^{f}, —C(O)OR^{e}, —NR^{c}C(O)R^{e}, —(C₁-C₄alkyl)C(O)R^{g}, —C(O)R^{g}, —C(O)NR^{c}R^{d}, NO₂, and —NR^{a}R^{b}, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo.

3. The compound of claim 1 or 2, wherein the compound is of the structural formula II: 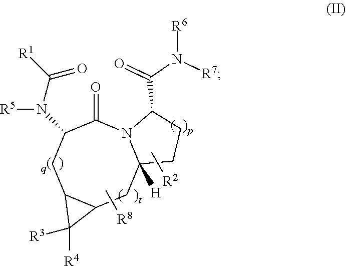
or a pharmaceutically acceptable salt thereof.

4. The compound of any one of claims 1 to 3, or a pharmaceutically acceptable salt thereof, wherein q is 1.

5. The compound of any one of claims 1 to 4, or a pharmaceutically acceptable salt thereof, wherein t is 1.

6. The compound of any one of claims 1 to 5, or a pharmaceutically acceptable salt thereof, wherein p is 1.

7. The compound of any one of claims 1 to 6, or a pharmaceutically acceptable salt thereof, wherein R² is hydrogen.

8. The compound of any one of claims 1 to 7, wherein the compound is of the structural formula III or IV: 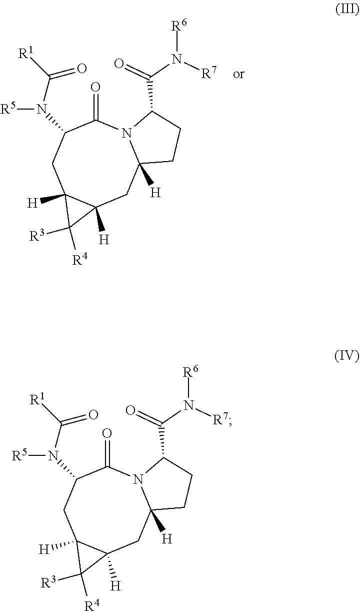
or a pharmaceutically acceptable salt thereof.

9. The compound of any one of claims 1 to 8, or a pharmaceutically acceptable salt thereof, wherein R⁵ is hydrogen.

10. The compound of any one of claims 1 to 9, or a pharmaceutically acceptable salt thereof, wherein R³ and R⁴ are each independently selected from hydrogen and halo.

11. The compound of any one of claims 1 to 10, or a pharmaceutically acceptable salt thereof, wherein R³ and R⁴ are each hydrogen.

12. The compound of any one of claims 1 to 10, or a pharmaceutically acceptable salt thereof, wherein R³ and R⁴ are each fluoro.

13. The compound of any one of claims 1 to 12, or a pharmaceutically acceptable salt thereof, wherein R¹ is selected from 8- to 10-membered fused bicyclic heteroaryl and aryl, each of which are substituted with —CR^{1a}R^{2a}P(O)OR^{1b}OR^{2b}, —CR^{1a}R^{2a}P(O)[NHR^{Ty}][NH(AA)C(O)OR^{T}], —CR^{1a}R^{2a}P(O)[NH(AA)C(O)OR^{T}]][NH(AA)C(O)OR^{T}], or —CR^{1a}R^{2a}P(O)[OR^{1b}][NH(AA)C(O)OR^{T}].

14. The compound of any one of claims 1 to 13, or a pharmaceutically acceptable salt thereof, wherein, R¹ is selected from benzothiophenyl and naphthalenyl, each of which are substituted with —CR^{1a}R^{2a}P(O)OR^{1b}OR^{2b}, —CR^{1a}R^{2a}P(O)[NHR^{Ty}][NH(AA)C(O)OR^{T}], —CR^{1a}R^{2a}P(O)[NH(AA)C(O)OR^{T}]][NH(AA)C(O)OR^{T}], or —CR^{1a}R^{2a}P(O)[OR^{1b}][NH(AA)C(O)OR^{T}].

15. The compound of any one of claims 1 to 14, or a pharmaceutically acceptable salt thereof, wherein, R¹ is selected from,

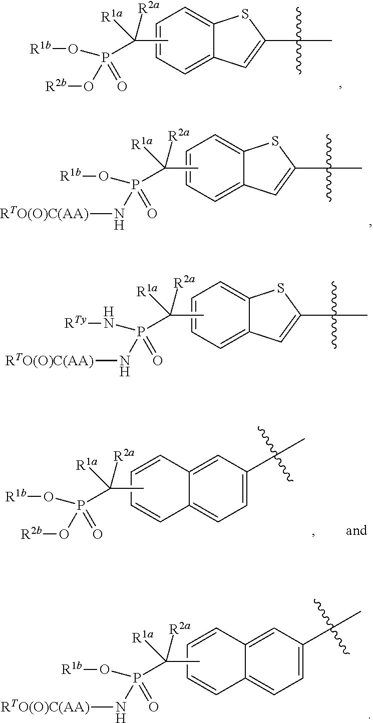

16. The compound of any one of claims 1 to 15, or a pharmaceutically acceptable salt thereof, wherein, R¹ is selected from,

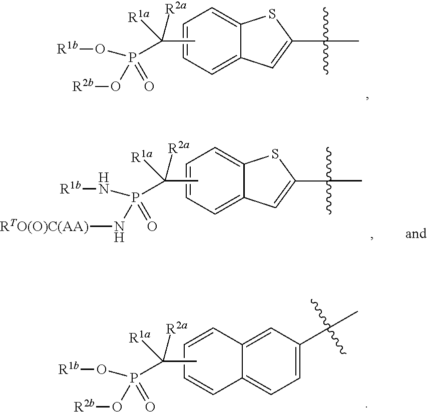

17. The compound of any one of claims 1 to 16, or a pharmaceutically acceptable salt thereof, wherein, R¹ is

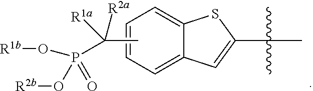

18. The compound of any one of claims 1 to 17, or a pharmaceutically acceptable salt thereof, wherein R^{1a} and R^{2a} are each independently selected from hydrogen and fluoro.

19. The compound of any one of claims 1 to 18, or a pharmaceutically acceptable salt thereof, wherein R^{1a} and R^{2a} are each hydrogen.

20. The compound of any one of claims 1 to 18, or a pharmaceutically acceptable salt thereof, wherein R^{1a} is hydrogen and R^{2a} is fluoro.

21. The compound of any one of claims 1 to 18, or a pharmaceutically acceptable salt thereof, wherein R^{1a} and R^{2a} are each fluoro.

22. The compound of any one of claims 1 to 21 or a pharmaceutically acceptable salt thereof, wherein R^{1b} and R^{2b} are each independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkylphenyl]-C(O)O—[(C₁-C₄)alkyl], —[(C₁-C₄)alkyl]-OC(O)—[NH(AA)C(O)OR^{T}], —[(C₁-C₄)alkyl]-OC(O)—[(C₁-C₄)alkyl]-OH, —[(C₁-C₄)alkyl]-OC(O)O-[5- to 7-membered heterocyclyl], —[(C₁-C₄)alkyl]-OC(O)O—[(C₁-C₄)alkyl]-O—[(C₁-C₄)alkyl], —[(C₁-C₄)alkyl]-OC(O)O—[(C₁-C₄)alkyl], —[(C₁-C₄)alkyl]-SC(O)—[(C₁-C₄)alkyl], —[(C₁-C₄)alkyl]-SC(O)—[(C₁-C₄)alkyl]-OH, and phenyl.

23. The compound of any one of claims 1 to 22, or a pharmaceutically acceptable salt thereof, wherein R^{1b} and R^{2b} are each independently selected from hydrogen, [(C₁-C₄)alkyl]-OC(O)—[(C₁-C₄)alkyl], —[(C₁-C₄)alkyl]-OC(O)O—[(C₁-C₄)alkyl]-O—[(C₁-C₄)alkyl], —[(C₁-C₄)alkyl]-OC(O)O—[(C₁-C₄)alkyl], and —[(C₁-C₄)alkyl]-SC(O)—[(C₁-C₄)alkyl].

24. The compound of any one of claims 1 to 23, or a pharmaceutically acceptable salt thereof, wherein R^{1b} and R^{2b} are each —[(C₁-C₄)alkyl]-OC(O)—[(C₁-C₄)alkyl].

25. The compound of any one of claims 1 to 23, or a pharmaceutically acceptable salt thereof, wherein R^{1b} and R^{2b} are hydrogen.

26. The compound of any one of claims 1 to 17 and 22, or a pharmaceutically acceptable salt thereof, wherein —CR^{1a}R^{2a}P(O)OR^{1b}OR^{2b} is selected from

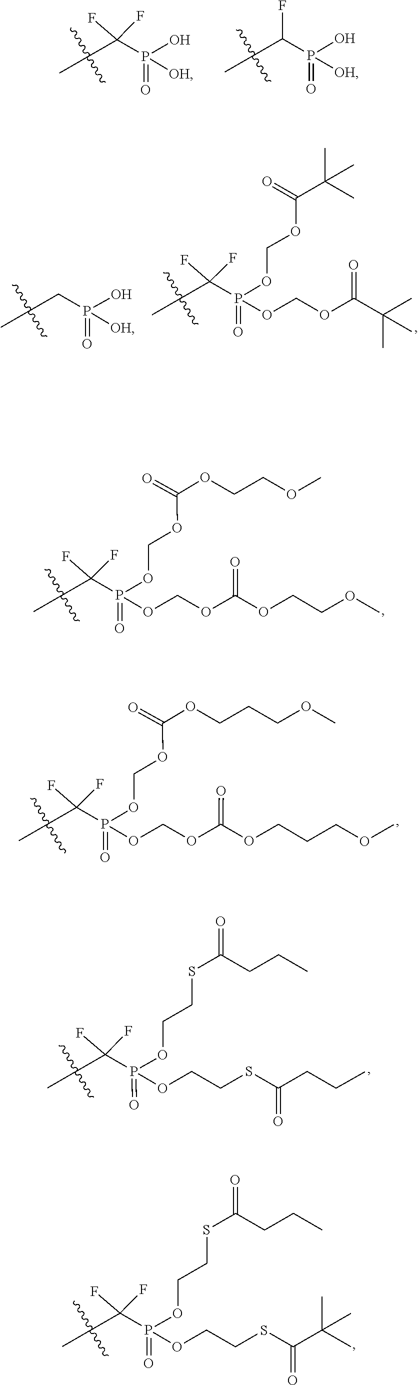

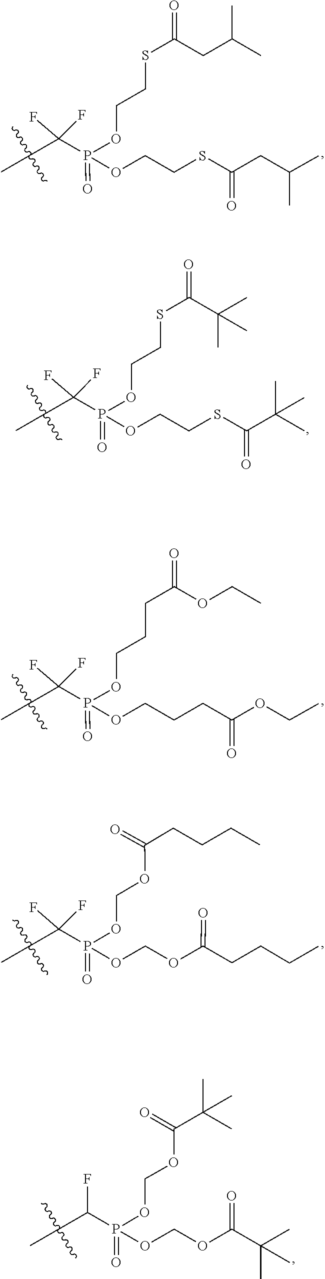 [SMILES: *C(F)(F)P(=O)(OCCSC(=O)CC(C)C)OCCSC(=O)CC(C)C ; *C(F)(F)P(=O)(OCCSC(=O)C(C)(C)C)OCCSC(=O)C(C)(C)C ; *C(F)(F)P(=O)(OCCCC(=O)OCC)OCCCC(=O)OCC ; *C(F)(F)P(=O)(OCOC(=O)CCCC)OCOC(=O)CCCC ; *C(F)P(=O)(OCOC(=O)C(C)(C)C)OCOC(=O)C(C)(C)C]

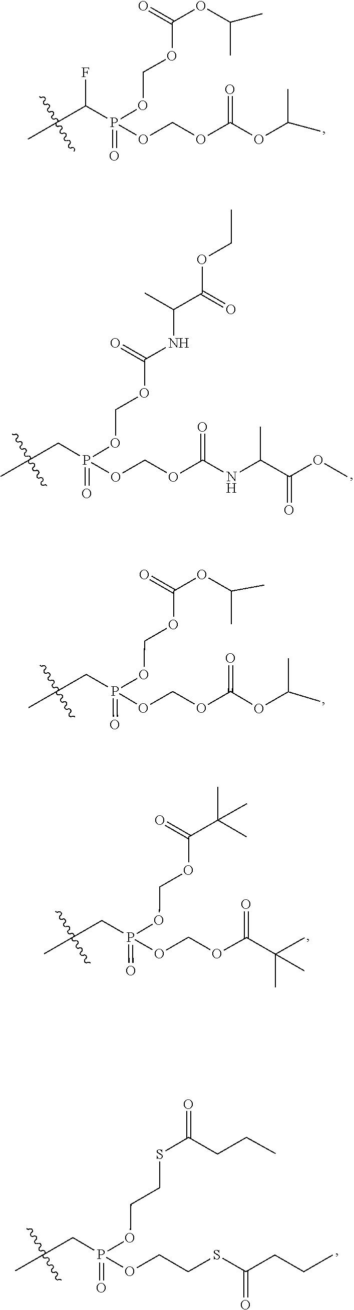 [SMILES: *C(F)P(=O)(OCOC(=O)OC(C)C)OCOC(=O)OC(C)C ; *CP(=O)(OCOC(=O)NC(C)C(=O)OC)OCOC(=O)NC(C)C(=O)OCC ; *CP(=O)(OCOC(=O)OC(C)C)OCOC(=O)OC(C)C ; *CP(=O)(OCOC(=O)C(C)(C)C)OCOC(=O)C(C)(C)C ; *CP(=O)(OCCSC(=O)CCC)OCCSC(=O)CCC]

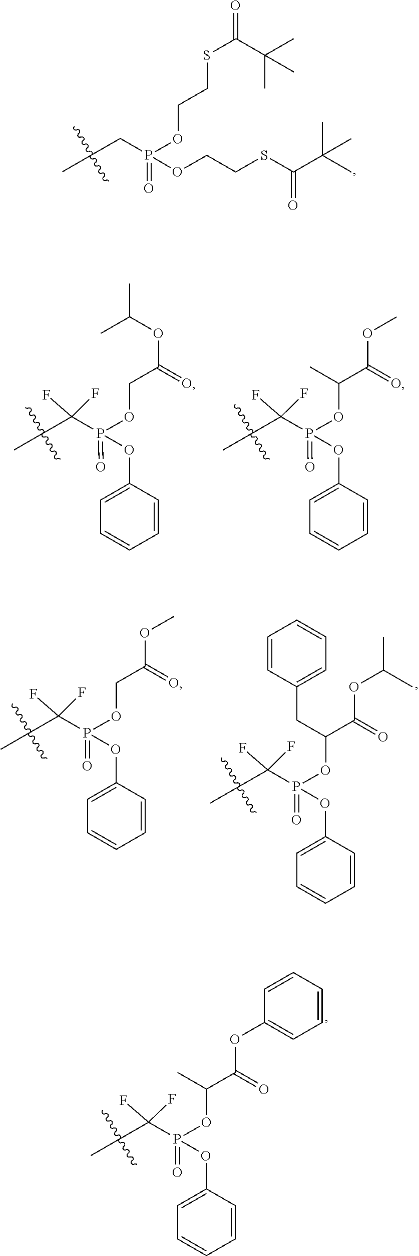 [SMILES: *CP(=O)(OCCSC(=O)C(C)(C)C)OCCSC(=O)C(C)(C)C ; *C(F)(F)P(=O)(OCC(=O)OC(C)C)Oc1ccccc1 ; *C(F)(F)P(=O)(Oc1ccccc1)OC(C)C(=O)OC ; *C(F)(F)P(=O)(OCC(=O)OC)Oc1ccccc1 ; *C(F)(F)P(=O)(Oc1ccccc1)OC(Cc1ccccc1)C(=O)OC(C)C ; *C(F)(F)P(=O)(Oc1ccccc1)OC(C)C(=O)Oc1ccccc1]

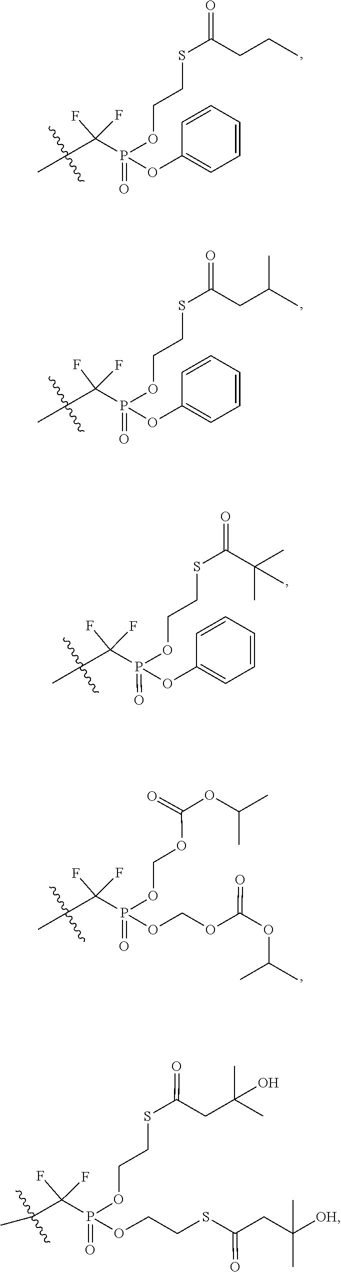 [SMILES: *C(F)(F)P(=O)(OCCSC(=O)CCC)Oc1ccccc1 ; *C(F)(F)P(=O)(OCCSC(=O)CC(C)C)Oc1ccccc1 ; *C(F)(F)P(=O)(OCCSC(=O)C(C)(C)C)Oc1ccccc1 ; *C(F)(F)P(=O)(OCOC(=O)OC(C)C)OCOC(=O)OC(C)C ; *C(F)(F)P(=O)(OCCSC(=O)CC(C)(C)O)OCCSC(=O)CC(C)(C)O]

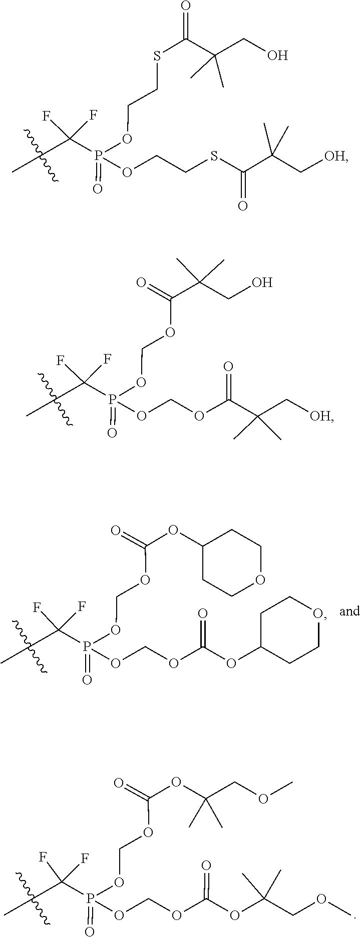 [SMILES: *C(F)(F)P(=O)(OCCSC(=O)C(C)(C)CO)OCCSC(=O)C(C)(C)CO ; *C(F)(F)P(=O)(OCOC(=O)C(C)(C)CO)OCOC(=O)C(C)(C)CO ; *C(F)(F)P(=O)(OCOC(=O)OC1CCOCC1)OCOC(=O)OC1CCOCC1 ; *C(F)(F)P(=O)(OCOC(=O)OC(C)(C)COC)OCOC(=O)OC(C)(C)COC]

27. The compound of any one of claims 1 to 17, 22, and 26, or a pharmaceutically acceptable salt thereof, wherein —CR^{1a}R^{2a}P(O)OR^{1b}OR^{2b} is

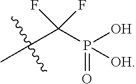 [SMILES: *C(F)(F)P(=O)(O)O]

28. The compound of any one of claims 1 to 16, or a pharmaceutically acceptable salt thereof, wherein -(AA)C(O)OR^{T} is —C(R′)(R)C(O)R^{T} or —C(R′)(R)CH₂C(O)R^{T}, wherein R′ is hydrogen or methyl and R is selected from hydrogen, methyl, ethyl, —CH₂CH(CH₃)₂, —CH₂OCH₃, benzyl, and —CH₂CH₂-phenyl.

29. The compound of any one of claims 1 to 16, or a pharmaceutically acceptable salt thereof, wherein -(AA)C(O)OR^{T} is —C(R′)(R)C(O)R^{T} or —C(R′)(R)CH₂C(O)R^{T}, wherein R′ is hydrogen and R is selected from hydrogen, methyl, —CH₂CH(CH₃)₂, benzyl, and —CH₂CH₂-phenyl.

30. The compound of any one of claims 1 to 16, 28, and 29, or a pharmaceutically acceptable salt thereof, wherein R^{T} is selected from (C₁-C₄)alkyl, (C₁-C₄)alkyl-C(O)O—C₁₋₄alkyl, and benzyl.

31. The compound of any one of claims 1 to 16, and 28-30, or a pharmaceutically acceptable salt thereof, wherein —CR^{1a}R^{2a}P(O)[OR^{1b}][NH(AA)C(O)OR^{T}] is selected from

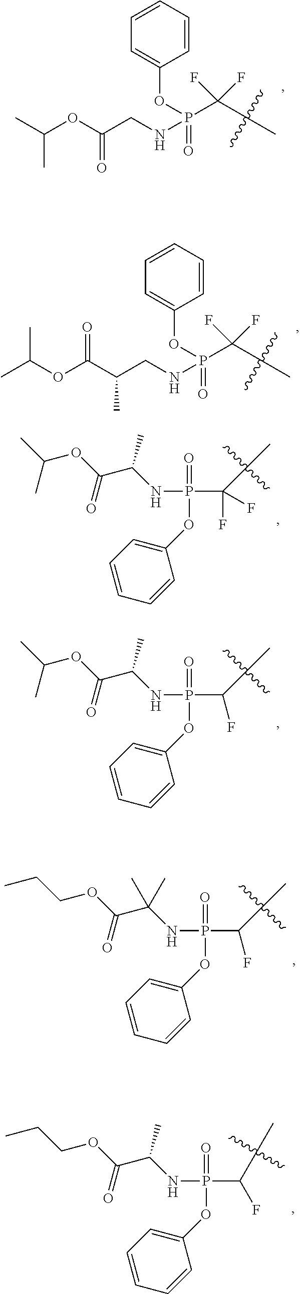

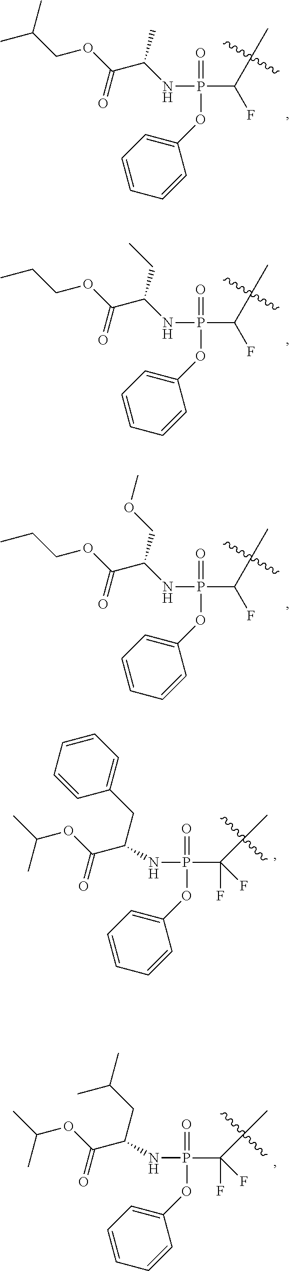 [SMILES: *C(F)P(=O)(N[C@@H](C)C(=O)OCC(C)C)Oc1ccccc1 ; *C(F)P(=O)(N[C@@H](CC)C(=O)OCCC)Oc1ccccc1 ; *C(F)P(=O)(N[C@@H](COC)C(=O)OCCC)Oc1ccccc1 ; *C(F)(F)P(=O)(N[C@@H](Cc1ccccc1)C(=O)OC(C)C)Oc1ccccc1 ; *C(F)(F)P(=O)(N[C@@H](CC(C)C)C(=O)OC(C)C)Oc1ccccc1]

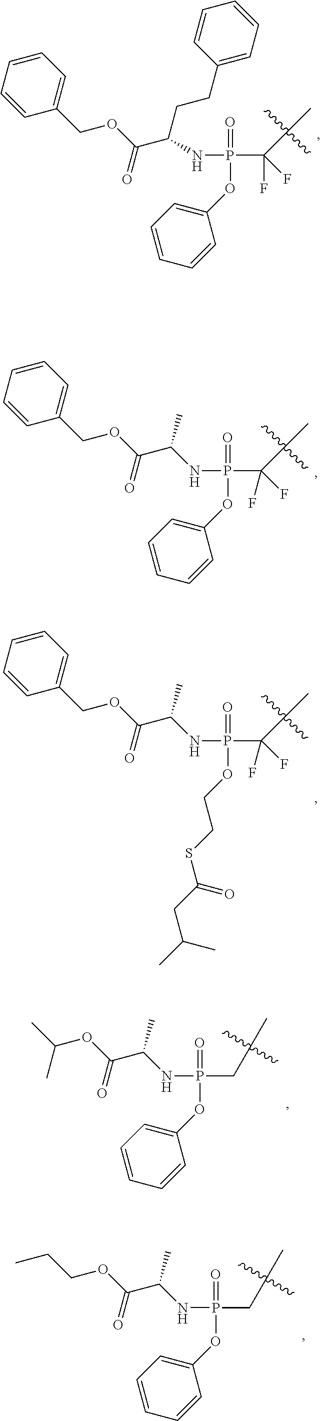 [SMILES: *C(F)(F)P(=O)(N[C@@H](CCc1ccccc1)C(=O)OCc1ccccc1)Oc1ccccc1 ; *C(F)(F)P(=O)(N[C@@H](C)C(=O)OCc1ccccc1)Oc1ccccc1 ; *C(F)(F)P(=O)(N[C@@H](C)C(=O)OCc1ccccc1)OCCSC(=O)CC(C)C ; *CP(=O)(N[C@@H](C)C(=O)OC(C)C)Oc1ccccc1 ; *CP(=O)(N[C@@H](C)C(=O)OCCC)Oc1ccccc1]

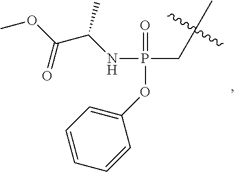 [SMILES: *CP(=O)(N[C@@H](C)C(=O)OC)Oc1ccccc1]

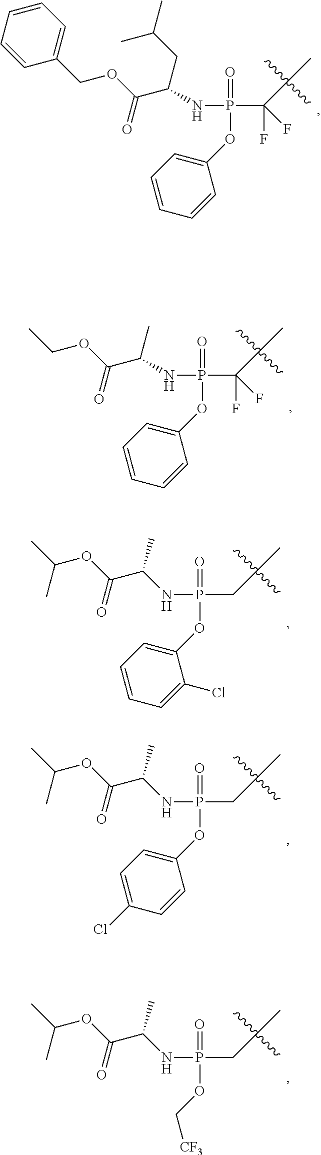

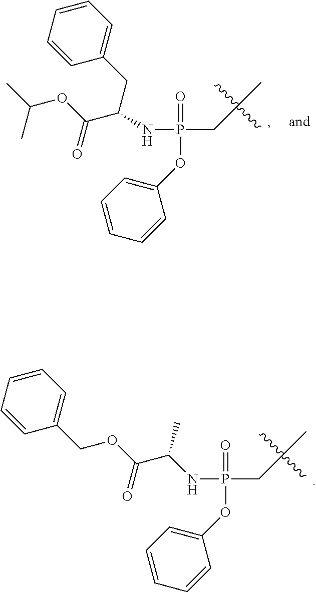 [SMILES: *CP(=O)(N[C@@H](Cc1ccccc1)C(=O)OC(C)C)Oc1ccccc1 ; *CP(=O)(N[C@@H](C)C(=O)OCc1ccccc1)Oc1ccccc1]

32. The compound of any one of claims 1 to 16, and 28-30, or a pharmaceutically acceptable salt thereof, wherein —CR^{1a}R^{2a}P(O)[OR^{1b}][NH(AA)C(O)OR^{T}] is selected from

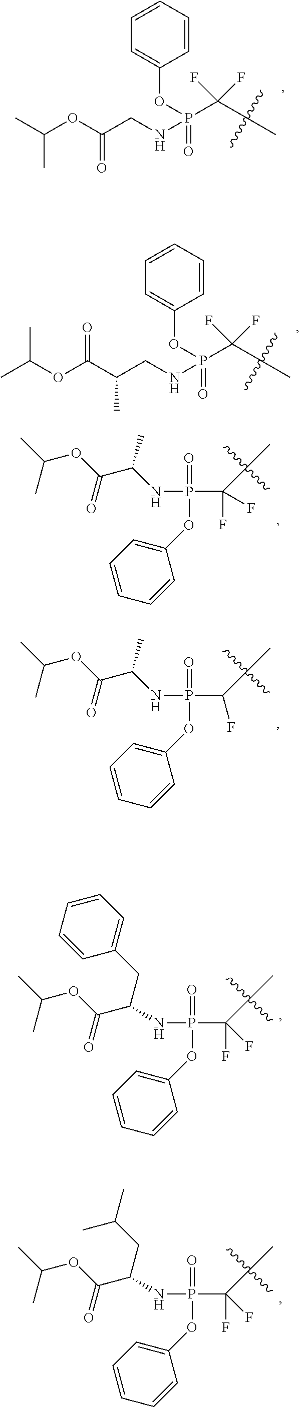

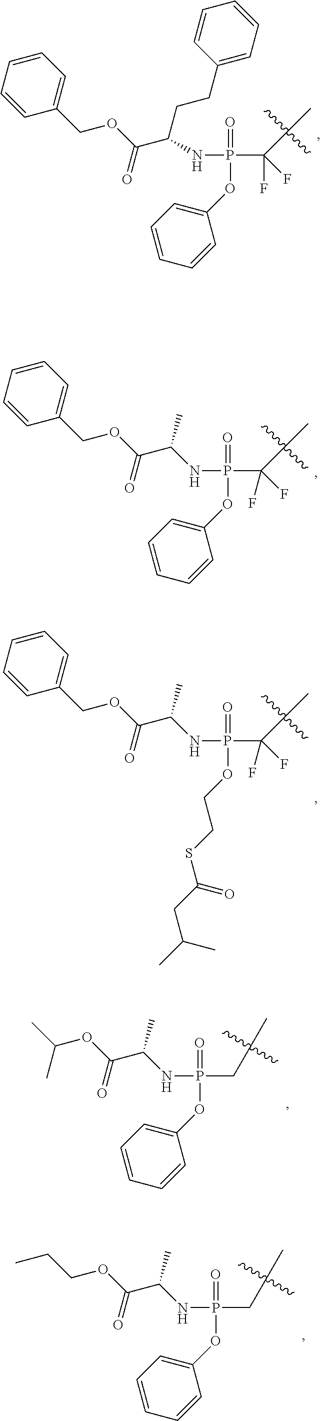 [SMILES: *C(F)(F)P(=O)(N[C@@H](CCc1ccccc1)C(=O)OCc1ccccc1)Oc1ccccc1 ; *C(F)(F)P(=O)(N[C@@H](C)C(=O)OCc1ccccc1)Oc1ccccc1 ; *C(F)(F)P(=O)(N[C@@H](C)C(=O)OCc1ccccc1)OCCSC(=O)CC(C)C ; *CP(=O)(N[C@@H](C)C(=O)OC(C)C)Oc1ccccc1 ; *CP(=O)(N[C@@H](C)C(=O)OCCC)Oc1ccccc1]

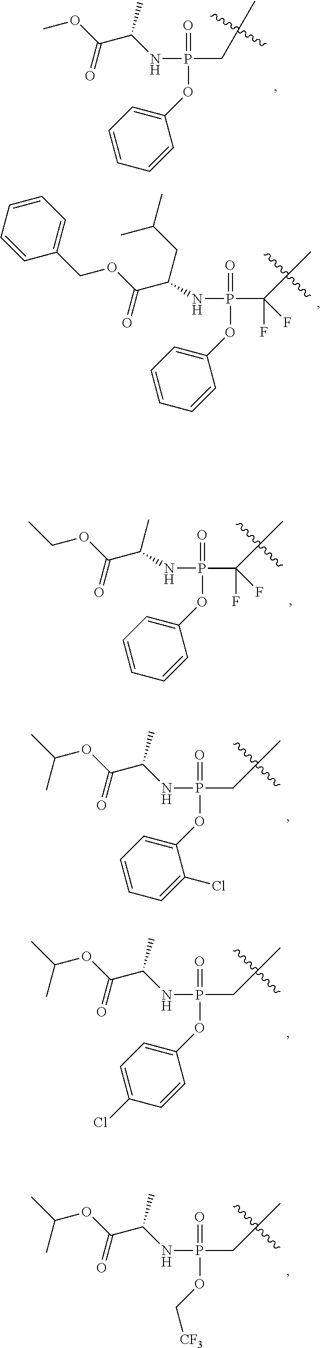

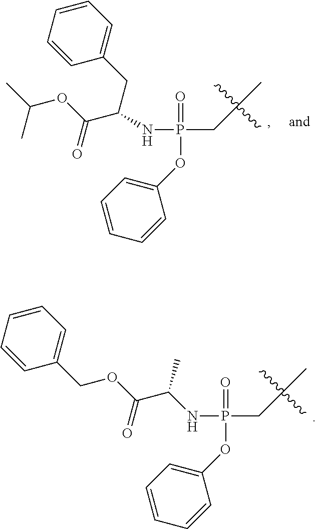 [SMILES: *CP(=O)(N[C@@H](Cc1ccccc1)C(=O)OC(C)C)Oc1ccccc1 ; *CP(=O)(N[C@@H](C)C(=O)OCc1ccccc1)Oc1ccccc1]

33. The compound of any one of claims 1 to 32, or a pharmaceutically acceptable salt thereof, wherein R⁶ is hydrogen.

34. The compound of any one of claims 1 to 33, or a pharmaceutically acceptable salt thereof, wherein R⁷ is selected from (C₁-C₄)alkyl, phenyl, and 4- to 6-membered monocyclic heterocyclyl, wherein said (C₁-C₄)alkyl is optionally substituted with, as valency permits, 1 to 3 groups selected from R^{Y} and said phenyl and 4- to 6-membered monocyclic heterocyclyl are each optionally substituted with, as valency permits, 1 to 3 groups selected from R^{Z}.

35. The compound of any one of claims 1 to 34, or a pharmaceutically acceptable salt thereof, wherein R⁷ is selected from (C₁-C₄)alkyl, phenyl, pyrrolidinyl, and azetidinyl, wherein said (C₁-C₄)alkyl is optionally substituted with, as valency permits, 1 to 3 groups selected from R^{Y} and said phenyl, pyrrolidinyl, and azetidinyl are each optionally substituted with, as valency permits, 1 to 3 groups selected from R^{Z}.

36. The compound of any one of claims 1 to 35, or a pharmaceutically acceptable salt thereof, wherein R^{Z} is selected from halo, —(C₁-C₄)alkylC(O)NR^{c}R^{d}, hydroxyl, phenyl, 4- to 6-membered heterocyclyl, 5- to 6-membered monocyclic heteroaryl, —C(O)NR^{c}R^{d}, and —C(O)R^{g}, wherein said phenyl is optionally substituted with, as valency permits 1 to 3 groups selected from halo, (C₁-C₄)alkyl, halo(C₁-C₄)alkyl, (C₁-C₄)alkoxy, and halo(C₁-C₄)alkoxy.

37. The compound of any one of claims 1 to 36, or a pharmaceutically acceptable salt thereof, wherein R^{Z} is selected from halo, —(C₁-C₄)alkylC(O)NR^{c}R^{d}, hydroxyl, phenyl, tetrahydropyran, tetrahydrofuran, oxetanyl, pyridinyl, pyrimidinyl, imidazoyl, triazoyl, pyrazolyl, pyridazinyl, —C(O)NR^{c}R^{d} and —C(O)R^{g}, wherein the pyridinyl, imidazoyl, and triazoyl are optionally substituted with one or two groups selected from halo and methyl.

38. The compound of any one of claims 1 to 36, or a pharmaceutically acceptable salt thereof, wherein R^{Z} is selected from halo, —(C₁-C₄)alkylC(O)NR^{c}R^{d}, hydroxyl, phenyl, tetrahydropyran, tetrahydrofuran, oxetanyl, pyridinyl, pyrazolyl, pyridazinyl, —C(O)NR^{c}R^{d} and —C(O)R^{g}.

39. The compound of any one of claims 1 to 38, or a pharmaceutically acceptable salt thereof, wherein R^{Y} is selected from hydroxyl and 5- to 10-membered monocyclic or bicyclic heteroaryl, wherein said 5- to 10-membered monocyclic or bicyclic is optionally substituted with, as valency permits, 1 to 3 groups selected from R^{X}.

40. The compound of any one of claims 1 to 39, or a pharmaceutically acceptable salt thereof, wherein R^{Y} is selected from hydroxyl, pyridinyl, and pyrrolopyridinyl.

41. The compound of any one of claims 1 to 40, or a pharmaceutically acceptable salt thereof, wherein R^{c} and R^{d} are each hydrogen.

42. The compound of any one of claims 1 to 41, or a pharmaceutically acceptable salt thereof, wherein R^{g} is —(C₁-C₄)alkyl.

43. The compound of any one of claims 1 to 32, or a pharmaceutically acceptable salt thereof, wherein R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, 1 to 3 groups selected from R^{Q}.

44. The compound of any one of claims 1 to 32 and 43, or a pharmaceutically acceptable salt thereof, wherein R⁶ and R⁷ together with the nitrogen atom to which they are attached form azetidinyl, 2,5-diazaspiro[3.4]octanyl, pyrrolidinyl, 2,6-diazaspiro[3.3]heptanyl, 2,6-diazabicyclo[3.2.0]heptanyl, piperazinyl, spiro[indoline-3,3′-pyrrolidine]yl, 6′,7′-dihydrospiro[azetidine-3,5′-pyrrolo[1,2-a]imidazole]yl, 3,4-dihydro-2H-benzo[b][1,4]oxazinyl, 3,4-dihydro-2H-pyrido[3,2-b][1,4]oxazine, 2,3-dihydro-1H-pyrido[2,3-b][1,4]oxazine, 2,3,4,5-tetrahydrobenzo[b][1,4]oxazepinyl, 1,2,3,4-tetrahydroquinoxalinyl, 1-azaspiro[3.5]nonanyl, 4-azaspiro[2.4]heptanyl, each of which being optionally substituted with, as valency permits, 1 to 3 groups selected from R^{Q}.

45. The compound of any one of claims 1 to 32 and 43, or a pharmaceutically acceptable salt thereof, wherein R⁶ and R⁷ together with the nitrogen atom to which they are attached form azetidinyl, 2,5-diazaspiro[3.4]octanyl, pyrrolidinyl, 2,6-diazaspiro[3.3]heptanyl, piperazinyl, spiro[indoline-3,3′-pyrrolidine]yl, 6′,7′-dihydrospiro[azetidine-3,5′-pyrrolo[1,2-a]imidazole]yl, each of which being optionally substituted with, as valency permits, 1 to 3 groups selected from R^{Q}.

46. The compound of any one of claims 1 to 32, and 43 to 45, or a pharmaceutically acceptable salt thereof, wherein R^{Q} is selected from halo, (C₁-C₄)alkyl, —OR^{e}, cyano, phenyl, hydroxyl, 4- to 6-membered heterocyclyl, 5- to 10-membered monocyclic or bicyclic heteroaryl, oxo, and, —C(O)R^{g}, wherein said (C₁-C₄)alkyl is optionally substituted with, as valency permits, 1 to 3 groups selected from R^{M}, and wherein said phenyl, 5- to 10-membered monocyclic or bicyclic heteroaryl, and 4- to 6-membered heterocyclyl are each optionally and independently substituted with, as valency permits, 1 to 3 groups selected from R^{F}, and wherein R^{e} is (C₁-C₄)alkyl or 5- to 6-membered heteroaryl.

47. The compound of any one of claims 1 to 32, and 43 to 45, or a pharmaceutically acceptable salt thereof, wherein R^{Q} is selected from halo, (C₁-C₄)alkyl, (C₁-C₄)alkoxy, cyano, phenyl, hydroxyl, 4- to 6-membered heterocyclyl, 5- to 10-membered monocyclic or bicyclic heteroaryl, oxo, and, —C(O)R^{g}, wherein said (C₁-C₄)alkyl is optionally substituted with, as valency permits, 1 to 3 groups selected from R^{M}, and wherein said phenyl, 5- to 10-membered monocyclic or bicyclic heteroaryl, and 4- to 6-membered heterocyclyl are each optionally and independently substituted with, as valency permits, 1 to 3 groups selected from R^{F}.

48. The compound of any one of claims 1 to 32 and 43 to 47, or a pharmaceutically acceptable salt thereof, wherein R^{Q} is selected from halo, (C₁-C₄)alkyl, —OR^{e}, cyano, phenyl, hydroxyl, morpholinyl, tetrahydropyranyl, thiomorpholinyl, piperidinyl, oxatanyl, pyrazolyl, pyridinyl, tetrazolyl, imidazolyl, pyrazinyl, isoxazoyl, oxazoyl, oxadiazolyl, triazolyl, pyrimidinyl, benzoimidazolyl, 1H-pyrrolo[3,2-c]pyridine, 2,4,5,6-tetrahydrocyclopenta[c]pyrazolyl, oxo, and, —C(O)R^{g}, wherein said (C₁-C₄)alkyl is optionally substituted with, as valency permits, 1 to 3 groups selected from R^{M}, and wherein said morpholinyl, tetrahydropyranyl, thiomorpholinyl, piperidinyl, oxatanyl, pyrazolyl, pyridinyl, tetrazolyl, imidazolyl, pyrazinyl, isoxazoyl, oxazoyl, oxadiazolyl, triazolyl, pyrimidinyl, benzoimidazolyl, 1H-pyrrolo[3,2-c]pyridine, and 2,4,5,6-tetrahydrocyclopenta[c]pyrazolyl are each optionally and independently substituted with, as valency permits, 1 to 3 groups selected from R^{F}, wherein R^{e} is (C₁-C₄)alkyl, pyridinyl, pyrazinyl, pyrimidinyl, pyrazole, and wherein the pyrazoyl represented by R^{e} is optionally substituted with (C₁-C₄)alkyl.

49. The compound of any one of claims 1 to 32 and 43 to 47, or a pharmaceutically acceptable salt thereof, wherein R^{Q} is selected from halo, (C₁-C₄)alkyl, (C₁-C₄)alkoxy, cyano, phenyl, hydroxyl, morpholinyl, tetrahydropyranyl, thiomorpholinyl, piperidinyl, oxatanyl, pyrazolyl, pyridinyl, tetrazolyl, imidazolyl, pyrazinyl, oxadiazolyl, triazolyl, pyrimidinyl, benzoimidazolyl, 2,4,5,6-tetrahydrocyclopenta[c]pyrazolyl, oxo, and, —C(O)R^{g}, wherein said (C₁-C₄)alkyl is optionally substituted with, as valency permits, 1 to 3 groups selected from R^{M}, and wherein said morpholinyl, tetrahydropyranyl, thiomorpholinyl, piperidinyl, oxatanyl, pyrazolyl, pyridinyl, tetrazolyl, imidazolyl, pyrazinyl, oxadiazolyl, triazolyl, pyrimidinyl, benzoimidazolyl, and 2,4,5,6-tetrahydrocyclopenta[c]pyrazolyl are each optionally and independently substituted with, as valency permits, 1 to 3 groups selected from R^{F}.

50. The compound of any one of claims 1 to 49, or a pharmaceutically acceptable salt thereof, wherein R^{g} is selected from, as valency permits, (C₁-C₄)alkyl, 4- to 6-membered heterocyclyl, and 5- to 6-membered heteroaryl, wherein said (C₁-C₄)alkyl is optionally substituted with, as valency permits, 1 to 3 groups selected from R^{J}, and said 4- to 6-membered heterocyclyl, and 5- to 6-membered heteroaryl are each independently optionally substituted with, as valency permits, 1 to 3 groups selected from (C₁-C₄)alkyl, (C₁-C₄)alkoxy, benzyl, and hydroxyl.

51. The compound of any one of claims 1 to 50, or a pharmaceutically acceptable salt thereof, wherein R^{J} is phenyl.

52. The compound of any one of claims 1 to 51, or a pharmaceutically acceptable salt thereof, wherein R^{g} is selected from, as valency permits, (C₁-C₄)alkyl, morpholinyl, azetidinyl, tetrahydropyranyl, oxatanyl, pyrrolidinyl, and pyrazolyl, wherein said (C₁-C₄)alkyl is optionally substituted with, as valency permits, 1 to 3 groups selected from R^{J}, and said morpholinyl, azetidinyl, tetrahydropyranyl, oxatanyl, pyrrolidinyl, and pyrazolyl are each independently optionally substituted with, as valency permits, 1 to 3 groups selected from (C₁-C₄)alkyl, (C₁-C₄)alkoxy, benzyl, and hydroxyl.

53. The compound of any one of claims 1 to 52, or a pharmaceutically acceptable salt thereof, wherein R^{F} is selected from halo, cyano, (C₁-C₄)alkyl, (C₁-C₄)haloalkyl, (C₁-C₄)alkoxy, hydroxyl, —N[(C₁-C₄)alkyl]₂, morpholinyl, piperazinyl, azetidinyl, pyrrolidinyl, and oxo, wherein said piperazinyl, pyrrolidinyl, and azetidinyl are each optionally substituted with 1 or 2 groups selected from cyano, halo, (C₁-C₄)alkyl, (C₁-C₄)alkoxy, and (C₁-C₄)alkyl(C₁-C₄)alkoxy.

54. The compound of any one of claims 1 to 52, or a pharmaceutically acceptable salt thereof, wherein R^{F} is selected from cyano, (C₁-C₄)alkyl, hydroxyl, and oxo.

55. The compound of any one of claims 1 to 54, or a pharmaceutically acceptable salt thereof, wherein R^{M} is selected from halo, hydroxy, (C₁-C₄)alkoxy, —S(O)₂R^{f}, —S(O)═NH(C₁-C₄)alkyl, pyridinyl, pyrazoyl, and phenyl optionally substituted with 1 or 2 halo.

56. The compound of any one of claims 1 to 54, or a pharmaceutically acceptable salt thereof, wherein R^{M} is selected from halo, (C₁-C₄)alkoxy, —S(O)₂R^{f}, and —S(O)═NH(C₁-C₄)alkyl.

57. The compound of any one of claims 1 to 55, or a pharmaceutically acceptable salt thereof, wherein R^{f} is (C₁-C₄)alkyl, and wherein said (C₁-C₄)alkyl is optionally substituted with, as valency permits, 1 to 3 halo.

58. The compound of claim 1, wherein the compound is selected from any one of Compound 1 to 435, or a pharmaceutically acceptable salt thereof.

59. A pharmaceutically acceptable composition comprising the compound of any one of claims 1 to 58, or a pharmaceutically acceptable salt thereof; and a pharmaceutically acceptable carrier.

60. A method of treating a condition responsive to the modulation of STAT3 or STAT6 in a subject comprising administering to the subject a therapeutically effective amount of the compound of any one of claims 1 to 58, or a pharmaceutically acceptable salt thereof, or the pharmaceutically acceptable composition of claim 59.
